SEQUENCE LISTING (574 sequences)

SEQ ID NO: 1 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAKYSRPDPSEGHVDLDYWGQGTLVTVSS

SEQ ID NO: 2 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAEYSRPDPSEGHVDLDYWGQGTLVTVSS

SEQ ID NO: 3 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAYYSRPDPSEGHVDLDYWGQGTLVTVSS

SEQ ID NO: 4 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAKYSRPDPSEGHVDLDYWGQGTLVTVSS

SEQ ID NO: 5 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAEYSRPDPSEGHVDLDYWGQGTLVTVSS

SEQ ID NO: 6 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAYYSRPDPSEGHVDLDYWGQGTLVTVSS

SEQ ID NO: 7 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAKYSRPDPSEGHVDLDYWGQGTLVTVSS

SEQ ID NO: 8 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAEYSRPDPSEGHVDLDYWGQGTLVTVSS

SEQ ID NO: 9 - protein, 132 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAYYSRPDPSEGHVDLDYWGQGTLVTVSS

SEQ ID NO: 10 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYTVRKYRYWGQGTLVTVSS

SEQ ID NO: 11 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYTVRKYRYWGQGTLVTVSS

SEQ ID NO: 12 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAYDALPYTVRKYRYWGQGTLVTVSS

SEQ ID NO: 13 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYTLRKYRYWGQGTLVTVSS

SEQ ID NO: 14 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYTLRKYRYWGQGTLVTVSS

SEQ ID NO: 15 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAYDALPYTLRKYRYWGQGTLVTVSS

SEQ ID NO: 16 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYTVRKYRYWGQGTLVTVSS

SEQ ID NO: 17 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYTLRKYRYWGQGTLVTVSS

SEQ ID NO: 18 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAEDALPYTLRKYRYWGQGTLVTVSS

SEQ ID NO: 19 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAYDALPYTLRKYRYWGQGTLVTVSS

SEQ ID NO: 20 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAEDALPYTVRKYRYWGQGTLVTVSS

SEQ ID NO: 21 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTADLQMNSLRPEDTALYYCAYDALPYTVRKYRYWGQGTLVTVSS

SEQ ID NO: 22 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYTLRKYRYWGQGTLVTVSS

SEQ ID NO: 23 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAEDALPYTLRKYRYWGQGTLVTVSS

SEQ ID NO: 24 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAYDALPYTLRKYRYWGQGTLVTVSS

SEQ ID NO: 25 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYTVRKYRYWGQGTLVTVSS

SEQ ID NO: 26 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAEDALPYTVRKYRYWGQGTLVTVSS

SEQ ID NO: 27 - protein, 121 aa, Synthetic construct
EVQLVESGGGVVQPGGSLRLSCAASGSTFSDYGVGWFRQAPGKGREFVADIDWEGEHTSYADSVKGRFTISRDNAKNTASLQMNSLRPEDTALYYCAYDALPYTVRKYRYWGQGTLVTVSS

SEQ ID NO: 28 - protein, 135 aa, Synthetic construct
EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAKYSRPDPSENHVDMDYWGKGTLVTVSSGCC

SEQ ID NO: 29 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAKYSRPDPSEGHVDLDYWGQGTLVTVSSGGC

SEQ ID NO: 30 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAEYSRPDPSEGHVDLDYWGQGTLVTVSSGGC

SEQ ID NO: 31 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAYYSRPDPSEGHVDLDYWGQGTLVTVSSGGC

SEQ ID NO: 32 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAKYSRPDPSEGHVDLDYWGQGTLVTVSSGGC

SEQ ID NO: 33 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAEYSRPDPSEGHVDLDYWGQGTLVTVSSGGC

SEQ ID NO: 34 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACAYYSRPDPSEGHVDLDYWGQGTLVTVSSGGC

SEQ ID NO: 35 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAKYSRPDPSEGHVDLDYWGQGTLVTVSSGGC

SEQ ID NO: 36 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAEYSRPDPSEGHVDLDYWGQGTLVTVSSGGC

SEQ ID NO: 37 - protein, 192 aa, Synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITTSFVPVFLPAKPTTTPAPRPPTPAPTIASQPLSLRPEASRPAAGGAVHTRGLDFAGGCGGHHHHHH

SEQ ID NO: 38 - protein, 230 aa, Synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 39 - protein, 215 aa, Synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 40 - protein, 234 aa, Synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 41 - protein, 215 aa, Synthetic construct; identical to SEQ ID NO: 39

SEQ ID NO: 42 - protein, 227 aa, Synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 43 - protein, 214 aa, Synthetic construct
DIVMTQSPSSLSASVGDRVTITCRTSRSISQALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 44 - protein, 135 aa, Synthetic construct
EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAYYSRPDPSEGHVDLDYWGQGTLVTVSSGGC

SEQ ID NO: 45 - RNA, 112 nt, Synthetic construct
  source 1..112: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - DNA, 1476 bp, Synthetic construct
  source 1..1476: mol_type = other DNA
atggacatgagggtgcccgcccagctcctgggcctgctgttactgtggctgcccggcgctaagtgccaggtacagctcgtggagagcggcggaggcgtggtgcagcccggcaggagcctgaggttaagctgcgccgccagcggcttcaccttcagcagctacggcatgcactgggtgagacaggcccccggcaagggcttggagtgggtggccatcatctactacgacggcagcaagaagtactacgccgacagcgtgaagggaaggttcaccatcagcagggacaacagcaagaacaccctgtacctgcagatgaacagcctgagggccgaggacaccgccgtgtactactgcgccagggagctgaccggcgacgccttcgacatctggggccagggaaccaaggtgaccgtcagcagcgggggcggaggatctggcggaggaggatcaggaggcggcggatctgggggcggcggaagcgagatcgtgctgacccagacccccagcagcctgagcgccagcctgggcgacagggtgaccatcagctgcagggcctcccagagcatcggctccagcctgcactggtaccagcagaagcccgaccagagccccaagctgctgatcaagtacgccagccagagcttcagcggcgtgcccagcaagttcatcggcagcggaagcggcaccgacttcaccttgaccatcaacagcctggaggccgaagacgccgccaccttttattgccaccagagcagcaccctgccctacaccttcggccagggcaccaagctggagatcaaggccgccgccatcgaggtgatgtaccctcccccttaccttgacaacgagaagagcaacggcaccatcatccacgtgaagggcaagcacctgtgccccagccccctgttccccggcccctctaagcccttctgggtcctggtggtggttggcggcgtgctggcctgctacagcctgctggtgaccgtggccttcatcatcttctgggtgaggagcaaaaggagcaggctgcttcacagcgactacatgaacatgacccccaggcgacccggccccaccaggaagcactaccagccctatgccccacccagggacttcgccgcctacaggtctagggtgaagttcagcaggagcgccgacgcccccgcctaccagcagggccagaaccagctgtacaatgaactgaacctgggcaggagggaggagtacgacgtgctggacaagaggaggggaagggaccccgagatgggcggcaagcccaggaggaagaacccccaggaaggcctgtacaacgagctccagaaggataaaatggccgaggcctacagcgaaatcggcatgaagggcgagaggaggaggggcaagggccacgacggcctttaccagggcctgagcaccgccaccaaggacacctacgacgccctgcacatgcaggccctgccccctaggtaatgatga

SEQ ID NO: 47 - DNA, 1437 bp, Synthetic construct
  source 1..1437: mol_type = other DNA
atggacatgagggtgcccgcccagctgctgggcctgttactcctttggctgcccggcgccaagtgccaggtgcagctggttgagagcggaggcggcgtggtgcagcccggcaggagcctgaggttaagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtacctgcagatgaacagcctgagagccgaggacaccgccgtgtactactgcgccagggagctgaccggcgacgccttcgacatctggggccagggaaccaaggtgaccgtgagcagcggcggcggaggatctggcggaggaggctcaggaggaggtggaagcgggggcggcggctctgagatcgtgctgacccagacccccagctctctgagcgccagcctgggcgacagggtgaccatcagctgcagggcctcccagagcatcggctctagcctgcactggtaccagcagaagcccgaccagagccccaagctgctgatcaagtacgccagccagagcttcagcggcgtgcccagcaagttcatcggcagcgggagcggcaccgacttcaccttgaccatcaacagcctggaggccgaggacgccgccaccttctactgccaccagagcagcaccctgccctacaccttcggccagggcaccaagctggagatcaaggccagcagcttaagacccgaagcctgcaggcccgccgccggaggagctgttcacaccaggggcttggattttgcttgcgatatctacatctgggcccccttagccggcacctgcggcgtgctgctgctgtctctggtgatcaccttatactgtaaccacaggaacgtgaggagcaagagaagcaggctgctgcacagcgactacatgaacatgacccctaggaggcccggccccaccaggaagcactaccagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgcttgacaagaggaggggaagggaccccgaaatgggcggcaagcccaggaggaaaaacccccaagagggcctgtacaacgagctccagaaggacaagatggccgaggcctacagcgagatcggcatgaagggcgagaggaggaggggcaagggccacgacggcctgtaccagggccttagcaccgccaccaaggacacctacgacgccctgcacatgcaggccctgccccctaggtaatgatga

SEQ ID NO: 48 - DNA, 1497 bp, Synthetic construct
  source 1..1497: mol_type = other DNA
atggacatgagggtgcccgcccagctgctgggactgctgctcctttggctgcctggcgccaagtgccaggtgcagctggtggagagcggcggaggagtggtgcagcccggcaggagcctgagactgagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtatctgcagatgaacagcctgagggccgaggataccgccgtgtactactgcgccagggagctgaccggcgacgccttcgacatctggggccagggaaccaaggtgaccgtgagcagcggaggcggaggatctggaggtggcggtagcggaggaggcggaagcggcggtggcggcagcgaaattgtgctgacccagacccccagcagcctcagcgccagcctgggcgacagggtgaccatcagctgcagggcctcccagagcatcggctccagcctgcactggtaccagcagaagcccgaccagagccccaagctgctgatcaagtacgccagccaaagcttcagcggcgtgcccagcaagttcatcggaagcggatccggaaccgacttcaccttaaccatcaacagcctggaggccgaggacgccgccaccttctactgtcaccagagcagcaccctgccctacacctttggccagggcaccaagctggaaatcaaagccagcaccaccacccccgctcctaggcctcccacacccgcccccacaattgctagccagcctttgagcctgaggcctgaagcttgtagacccgctgccggaggcgccgtgcacaccaggggcctggacttcgcttgcgacatctacatctgggcccccttggccggcacctgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctataggagcagggtgaagttcagcaggagcgccgacgcccccgcctaccagcagggccagaaccagctgtacaatgagctgaacctgggcaggagggaggagtacgacgtgttagacaagaggaggggaagggatcccgagatgggcggcaagcccaggaggaagaacccccaggaaggcctgtacaacgagctccagaaggacaagatggctgaggcctacagcgaaatcggaatgaagggcgagaggaggaggggcaagggccacgacggcctgtatcagggcctgagcaccgccaccaaggacacctacgacgccctgcacatgcaggccctgccccctaggtaatgatga

SEQ ID NO: 49 - DNA, 1557 bp, Synthetic construct
  source 1..1557: mol_type = other DNA
atggacatgagggtgcccgcccaactgcttggcctgctgctcctttggctgcccggcgccaagtgccaggtgcagctggtggagtctggcggaggcgtggtgcagcccggaaggagcctgagactgagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtatctgcagatgaacagcctgagggccgaggataccgccgtgtactactgcgccagggagctgaccggcgacgccttcgacatctggggccagggaaccaaggtgaccgtgagcagcggcggaggaggatctggaggcggcggatcaggaggtgggggaagcgggggaggcggcagcgaaatcgtgctgacccagacccccagcagcctcagcgccagcctgggcgacagggtgaccatcagctgcagggcctcccagagcatcggctccagcctgcactggtaccagcagaagcccgaccagagccccaagctgctgatcaagtacgccagccaaagcttcagcggcgtgcccagcaagttcatcggcagcggaagcggcaccgacttcaccttgaccatcaacagcctggaggccgaggacgccgccaccttctactgccaccagagcagcaccctgccctacaccttcggccagggcaccaagctggagatcaaggccagcctgagcaacagcatcatgtacttcagccacttcgtgcccgtgttcctgcccgccaagcctaccacaacacccgcccccaggcccccaacacctgctcccacaatcgccagccagcccttatccttaaggcccgaggcctgcagacccgctgccggaggcgctgtgcataccaggggactggacttcgcttgcgacatctacatctgggcccccttagccggaacatgcggcgtgctgctgctgagcctggtgatcaccctgtactgcaaccacaggaacgtgaggagcaagaggagcaggctgctgcacagcgactacatgaacatgacccccaggcgacccggccctaccaggaaacattatcagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggaaagcccaggaggaagaacccccaggagggcctgtacaacgagctccagaaggacaagatggccgaggcctatagcgagatcggcatgaagggcgagaggaggaggggcaagggccacgacggcctgtaccaaggcctgagcaccgccaccaaggacacctacgacgctcttcacatgcaggccctgccccctaggtaatgatga

SEQ ID NO: 50 - DNA, 1476 bp, Synthetic construct
  source 1..1476: mol_type = other DNA
atggacatgagggtgcctgcccagctgctgggcctgctgctgctgtggcttcctggcgccaagtgccaggtgcagctggtgcagagcggcgccgaggtgaagaagcctggcgcctccgtgaaggtgagctgcaaggccagcggctacaccttcaccggctactacatccactgggtgagacaggcccccggccagggactggagtggatgggctggatcaaccccaacagcggcggcaccaactacgcccagaagttccagggcagggtgacaatgaccagggacaccagcatcggcaccgcctacatggagctgagcaggctcaggagcgacgacaccgccatctactactgcaccagggaggacacctggaactacatcgactactggggccagggaaccctggtgacagtgagcagcggaggtggcggaagcggcggcggaggatcagggggaggtggaagtggcggaggaggcagcgacatccagatgacccagagccccagctccctgagcgccagcgtgggcgacagggtgaccatcacctgcagggcctcccagagcatcagcagccacctgaactggtaccagcagaagcccggcaaggcccccaagctgctgatctacgccgccagcagcctccacagcggcgtgcccagcaggttctctggctctggcagcggcaccgacttcaccttaacaatttctagcctgcaacccgaggacttcgctacctactgctgccagcagagctacagcacccccagaacctttggccagggcaccaaggtggagatcaaggccgccgccatcgaggtgatgtaccctcccccttacctggacaacgagaagagcaacggcaccatcatccacgtgaagggcaagcacctgtgccccagccccctgttccccggcccctctaagcccttctgggtcctggtggtggtgggcggcgtgctggcctgctatagcctgctggtgaccgtggccttcatcatcttctgggtgaggagcaagaggagcaggctgctgcacagcgattacatgaacatgacccccaggcgacccggccctaccaggaagcactaccagccctacgccccacccagggacttcgccgcctacaggagcagggtgaagttcagcaggagcgccgacgcccccgcttaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaagacaagatggccgaggcctacagcgagatcggcatgaagggcgagaggaggaggggcaagggccacgacggcctgtaccagggcctgagcaccgccaccaaggatacctacgatgccctgcacatgcaggccctgcctcctaggtaatgatga

SEQ ID NO: 51 - DNA, 1437 bp, Synthetic construct
  source 1..1437: mol_type = other DNA
atggacatgagggtgcccgcccagctgctgggcctgctgttgctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagggtgacaatgaccagggacaccagcatcggcaccgcctacatggagctgagcaggctcaggagcgacgacaccgccatctactactgcaccagggaggacacctggaactacatcgactactggggccagggaaccctggtgaccgtgagcagcggagggggaggaagcggcggaggaggcagtggaggaggaggatccggaggcggcggaagcgacatccagatgacccagagccccagctccctgagcgccagcgtgggcgacagggtgaccatcacctgcagggcctctcagagcatcagctctcacctgaattggtaccagcagaagcccggcaaggcccccaagctgctgatctacgccgccagcagcttacacagcggcgtgcccagcaggttttctggcagcggaagcggcaccgacttcaccttaacaatctccagcctgcagcccgaggactttgctacctactgctgccagcagagctacagcacccccagaaccttcggccagggcaccaaggtggaaatcaaggcctcttctctgagacccgaggcttgcagacccgccgctggaggcgccgttcataccaggggcctggacttcgcttgcgacatctacatctgggcccccttggccggcacctgcggcgtgctgctgctgagcctggtgatcaccctgtactgcaaccacaggaacgtgaggagcaagaggagcaggctgctgcatagcgactacatgaacatgacccccaggcgacccggccccaccaggaagcactaccagccctacgccccacccagggacttcgccgcctacaggagcagggtgaagttcagcaggagcgctgatgcccccgcctaccagcagggccagaaccagctgtacaatgagctgaatctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggagaggcaagggccacgacggcctgtaccagggcctgagcaccgccaccaaagacacctacgacgccctgcacatgcaggccctgccccctaggtaatgatga

SEQ ID NO: 52 - DNA, 1497 bp, Synthetic construct
  source 1..1497: mol_type = other DNA
atggacatgagggtgcccgcccagctgctgggactgctgctcctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagagtgaccatgaccagggacaccagcatcggcaccgcctacatggagctgagcaggctcaggagcgacgacaccgccatctactactgcaccagggaggacacctggaactacatcgactactggggccagggcaccctggtgaccgtgagcagcggaggaggcggatctggcggcggaggatcaggagggggaggaagcggaggcggaggcagcgacatccagatgacccagagccccagctccctgagcgccagcgtgggcgacagggttaccattacctgcagggcctctcagtctatcagctctcatctgaactggtaccagcagaagcccggcaaggcccctaagctgctgatctatgccgccagcagcctccacagcggcgtgcctagcaggttctctggcagcggaagtggcaccgacttcaccttaaccatcagctcactgcagcccgaggacttcgctacctactgctgccagcagagctacagcacccccagaaccttcggacagggcaccaaggtggagatcaaggcctccacaacaacacccgctcccaggccccctacccccgcccccacaattgccagccaacccctcagcctgagacccgaagcctgtagacctgccgcagggggagccgtgcacaccaggggcctggacttcgcatgcgacatctacatctgggcccccttagccggcacctgcggcgtgctgctgctgagccttgtgatcaccctgtactgcaaccacaggaacgtgaggagcaagaggagcaggctgctgcacagcgactacatgaacatgacccccaggcgacccggccccaccaggaagcattaccagccctacgccccacccagggacttcgccgcctacaggagcagggtgaagttcagcaggagcgccgacgcccctgcctaccagcagggccagaaccagctgtacaatgagctgaacctgggcaggagggaggagtatgacgtgctggacaagaggaggggaagggaccccgagatgggcggcaagcccaggaggaagaacccccaggagggcctgtataacgagctccagaaggacaagatggccgaagcctacagcgagatcggcatgaagggcgagaggaggaggggcaagggccatgacggcctgtaccagggcctgagcaccgccaccaaggacacctacgacgccctgcacatgcaggccctgccccctaggtaatgatga

SEQ ID NO: 53 - DNA, 1557 bp, Synthetic construct
  source 1..1557: mol_type = other DNA
atggacatgagggtgcccgcccagctgctgggactgctgctcctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagagtgaccatgaccagggacaccagcatcggcaccgcctacatggagctgagcaggctcaggagcgacgacaccgccatctactactgcaccagggaggacacctggaactacatcgactactggggccagggcaccctggtgaccgtgagcagcggaggaggcggatctggcggcggaggatcaggagggggaggaagcggaggcggaggcagcgacatccagatgacccagagccccagctccctgagcgccagcgtgggcgacagggttaccattacctgcagggcctctcagtctatcagctctcatctgaactggtaccagcagaagcccggcaaggcccctaagctgctgatctatgccgccagcagcctccacagcggcgtgcctagcaggttctctggcagcggaagtggcaccgacttcaccttaaccatcagctcactgcagcccgaggacttcgctacctactgctgccagcagagctacagcacccccagaaccttcggacagggcaccaaggtggagatcaaggcctccacaacaactagcatcatgtacttcagccatttcgtgcccgtgtttcttcccgctaaacccaccactacacccgctcccaggccccctacccccgcccccacaattgccagccaacccctcagcctgagacccgaagcctgtagacctgccgcagggggagccgtgcacaccaggggcctggacttcgcatgcgacatctacatctgggcccccttagccggcacctgcggcgtgctgctgctgagccttgtgatcaccctgtactgcaaccacaggaacgtgaggagcaagaggagcaggctgctgcacagcgactacatgaacatgacccccaggcgacccggccccaccaggaagcattaccagccctacgccccacccagggacttcgccgcctacaggagcagggtgaagttcagcaggagcgccgacgcccctgcctaccagcagggccagaaccagctgtacaatgagctgaacctgggcaggagggaggagtatgacgtgctggacaagaggaggggaagggaccccgagatgggcggcaagcccaggaggaagaacccccaggagggcctgtataacgagctccagaaggacaagatggccgaagcctacagcgagatcggcatgaagggcgagaggaggaggggcaagggccatgacggcctgtaccagggcctgagcaccgccaccaaggacacctacgacgccctgcacatgcaggccctgccccctaggtaatgatga

SEQ ID NO: 54 - DNA, 1488 bp, Synthetic construct
  source 1..1488: mol_type = other DNA
atggacatgagggtgcctgcccagttgttgggcctgctgctgctgtggctgcccggcgccaagtgccaggttaccctgagggagtctggccccgccctggtgaagcccacccagaccttaaccctgacctgcaccttcagcggcatcagcctgaccaccagcggcatgtgcgtgagctggatcaggcagcccccaggcaaggccctggagtggctggccctgatcgactgggacggcgacaactactacagcaccagcctgaagaccaggctgaccatctccaaggacaccagcaagaaccaggtggtgctgaccatggccagcatggaccccgtggacaccgccacatactactgcgccaggatgggctacacctacggctacgacgctttcgacatctggggccagggcaccatggtgacagtgagcagcggcggtggaggatctggcggaggcggaagcggaggaggcggatctgggggcggaggaagcgagattgtgctgacccagacccccagcagcctcagcgccagcctgggcgacagggtgaccatcagctgcagggcctctcagagcatcggcagcctgttacactggtaccagcagaagcccgaccagagccccaagctgctgatcaagttcgccagccagagcctgagcggcgtgcccagcaggttctctggctctggcagcggcaccgacttcaccttaaccatcaacagcctggaggccgaagacgccgccacctactactgccaccagagcaggagcctgccctttaccttcggccccggcaccaaggttgacatcaacgccgccgccatcgaggtgatgtaccctcccccttacctggacaacgagaagagcaacggcaccatcatccacgtgaagggcaagcacctgtgccccagccccctgttccccggcccctctaagcccttctgggttctggtggtggtgggcggcgtgctggcctgctacagcctgctggtgaccgtggccttcatcatcttctgggtgaggagtaagaggagcaggctgctgcacagcgactacatgaacatgacccccaggcgacccggccccaccaggaagcactaccagccctacgcccctccaagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaaaatggccgaagcctacagcgagatcggcatgaagggcgagaggaggaggggcaaaggccacgacggcctgtaccagggcctgagcaccgccaccaaagacacctacgacgccctgcacatgcaggccctgccccctaggtaatgatga

SEQ ID NO: 55 - DNA, 1449 bp, Synthetic construct
  source 1..1449: mol_type = other DNA
atggacatgagggtgcccgcccagctgctgggcctgctgttactttggctgcccggcgccaagtgccaagtgaccctgagggagtctggccccgccctggtgaagcccacccagaccctcaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctccaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagcagcggcggaggaggatctggaggcggaggctctggtggaggaggcagcgggggcggaggaagcgagattgtgctgacacagacccccagctccctgagcgccagcctgggcgacagggtgaccatcagctgcagggcctcccagagcatcggcagcctgttgcactggtaccagcagaagcccgatcagagccccaaactgctgatcaagttcgccagccagagcctgagcggcgtgcccagcaggttctctggaagcggctctggcaccgacttcaccttaaccatcaacagcctggaggccgaggacgccgccacctactactgccaccaaagcaggagcctgccctttaccttcggccccggcaccaaggtggacatcaacgccagcagcctcaggcccgaagcttgtaggcctgctgccggcggcgctgttcacaccaggggcttagactttgcttgcgacatctacatctgggcccccttagccggaacctgcggcgtgctgctgctgagcctggtgatcaccctgtactgcaaccacaggaatgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaatcagctgtacaatgagctgaacctgggcaggagggaggagtacgacgtgctggacaagaggaggggaagggaccctgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcttaagcaccgccaccaaggatacctacgacgccctgcacatgcaggccctgccccctaggtaatgatga

SEQ ID NO: 56 - DNA, 1509 bp, Synthetic construct
  source 1..1509: mol_type = other DNA
atggacatgagggtgcccgcccagcttctgggcctgctgctcttgtggctgcctggcgccaagtgccaggtgaccctgagggagtctggccccgccctggtgaagcccacccagaccttgaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctctaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagctctggcgggggaggaagcggcggcggaggatctggtggaggtgggtctggaggcggcggcagcgaaatcgtgctgacccagacccccagcagcctcagcgccagcctgggcgacagggtgaccatcagctgcagggcctcccagagcatcggcagcctgttgcactggtaccagcagaagcccgaccagagccccaagctgctgatcaagttcgccagccagagcctgagcggcgtgcccagcaggttctctggaagcggctctggaaccgacttcaccttaaccatcaacagcctggaggccgaagacgccgccacctattactgccaccagagcaggagcctgcccttcaccttcggacccggaaccaaggtggacatcaacgccagcacaaccacacccgcccctaggccccctacccccgcccccacaatcgctagccaacccctgagccttaggcccgaagcctgtagacctgccgccggaggcgccgtgcataccaggggcctggacttcgcttgtgacatctacatctgggcccccttagccggaacctgcggcgtgctgctgctgagcctggtgatcaccctgtactgcaaccacaggaacgtgaggagcaaaaggagcaggctgctgcacagcgactacatgaacatgacccccaggaggcccggccccaccaggaagcactaccagccctacgccccacccagggacttcgccgcctacaggagcagggtgaagtttagcaggagcgccgacgcccccgcctaccagcagggccagaatcagctgtacaatgaattaaacctgggcaggagggaggagtacgacgtgctggataagaggaggggaagggaccccgagatgggcggcaagcccaggaggaagaaccctcaggagggactgtacaacgagctccagaaggataagatggccgaggcctacagcgagatcggcatgaagggcgagaggaggaggggcaagggccacgacggcctgtaccaaggcctgagcaccgccaccaaggacacctacgacgccctgcacatgcaggccctgccccctagataatgatga

SEQ ID NO: 57 - DNA, 1569 bp, Synthetic construct
  source 1..1569: mol_type = other DNA
atggacatgagggtgcccgcccaactgctgggcctgctgctcctttggctgcctggcgccaagtgccaggtgaccctgagggagtctggccccgccctggtgaagcccacccagaccttgaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctctaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagcagcggcggaggcggttctggtggcggaggctcaggaggcggaggatctggaggcggcggcagcgaaatcgtgctgacccagacccccagcagcctcagcgccagcctcggcgacagggtgaccatcagctgcagggcctcccagagcatcggcagcctgctgcactggtaccagcagaagcccgaccagagccccaagctgctgatcaagttcgccagccagagcctgagcggcgtgcccagcaggttctctggcagcggctctggcaccgacttcaccttaaccatcaacagcctggaggccgaggacgccgccacctactactgccatcaaagcaggagcctgcccttcaccttcggccccggcacaaaagtggacatcaacgccagcctgagcaacagcatcatgtactttagccattttgtgcccgtgttcctgcctgccaagcccacaacaacccccgcccccaggcctcctacccctgcccctacaatcgctagccaacccctgtctctgagacccgaggcctgcagacctgctgccggaggcgccgtgcacaccaggggactggacttcgcttgcgacatctatatctgggcccccttagccggcacctgcggcgtgctgctgctgagcctggtgattaccctgtactgcaaccacaggaacgtgaggagcaagaggagcagactgctgcacagcgactacatgaacatgacccccaggcgacccggccccaccaggaagcactaccagccctacgccccacccagggacttcgccgcctacaggagcagggtgaagttcagcaggtcagccgacgcccccgcctaccaacagggccagaaccagctgtacaatgagctgaacctgggcaggagggaggagtacgacgtgctggacaagaggaggggaagggatcctgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgctaccaaggacacctacgacgccctgcacatgcaggccctgccccctaggtaatgatga

SEQ ID NO: 58 - DNA, 1488 bp, Synthetic construct
  source 1..1488: mol_type = other DNA
atggacatgagggtgcccgcccagctgctgggcctgttactgctgtggctgcccggcgctaagtgccaggtgaccctgagggagtctggccccgccctggtgaagaccacccagaccctgacactgacctgcaccttcagcggcttcagcctgtccaccagcggcatgtgcgtgagctggatcaggcagcccccaggcaaggccttggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggaaccctggtcaccgtgagcagcggaggcggtggatctggaggcggaggaagcggcggaggaggatccggaggcggcggcagcgatatccagatgacccagagccccagctccctgagcgcctctgtgggcgacagggtgaccatcacctgcagggcctctcagaggatcagctctcagctgaactggtaccagcagaagcccggcaaggcccccaagctgctgatctacgccgccagcagcctccagagcggcgtgccctctaggttctctggctctggaagcggcaccgagttcaccctgaccattagcagcttgcagcccgaggacatcgccacctactgctgccagcagacctacagcaagcctatcaccttcggccagggcaccaggctggagatcaaggccgccgccatcgaggtgatgtaccctcccccttacctggacaacgagaagagcaacggcaccatcatccacgtgaagggcaagcacctgtgccccagccccctgttccccggcccctctaagcccttctgggtcctggtggtggtgggcggcgtgctggcctgctacagcctgctggtgaccgtggccttcatcatcttctgggtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaggccttaccccctaggtaatgatga

SEQ ID NO: 59 - DNA, 1449 bp, Synthetic construct
  source 1..1449: mol_type = other DNA
atggacatgagggtgcccgcccaactgctgggcctgttgctgctttggctgcccggcgccaagtgccaggtgaccctgagggagtctggacccgccctggtgaagaccacccagaccctgacactgacctgcaccttcagcggcttcagcctgtccaccagcggcatgtgcgtgagctggatcaggcagccccctggcaaggccttggagtggctggccctgatcgactgggacgacgacaagtactacagcaccagcctgaagaccaggctgaccatcagcaaggacaccagcaagagccaggtggtgctgaccatgaccagcatggaccccgtggacaccgccacatactactgcgccaggatcaggtacatctacggctacgactacttcgactactggggccagggcaccctggtgaccgtgagcagcggaggcggaggatctggcggagggggatctggaggtggaggctctggcggcggcggcagcgatatccagatgacccagagccccagctccctgagcgccagcgtgggcgacagggtgaccatcacctgcagggcctcccagaggatcagctcccagctgaactggtaccagcagaagcccggcaaggcccccaagctgctgatctacgccgccagcagccttcagagcggcgtgcccagcaggttctccggcagcgggagcggcaccgagttcaccctgaccattagctcactgcagcccgaggacatcgccacctactgctgccagcagacctacagcaagcccatcaccttcgggcagggcaccaggctggagatcaaggcctcttctttgagacccgaggcttgtaggcccgcagccggcggcgccgtgcacacaaggggactggatttcgcttgcgacatctatatctgggcccccttagccggcacctgcggcgtgctgctgctgagcctggtgatcaccctgtactgcaaccacaggaacgtgaggagcaagaggagcaggctgctgcacagcgactacatgaacatgacacccaggaggcccggccctaccaggaagcactaccagccctacgccccacccagggacttcgccgcttataggagcagggtgaagttcagcaggagcgccgacgctcccgcctaccagcagggccagaaccagctgtacaatgagctgaacctgggcaggagggaggagtacgacgtgctggacaagagaaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaagacacctacgacgccctgcacatgcaggccctgccccctaggtaatgatga

SEQ ID NO: 60 - DNA, 1509 bp, Synthetic construct
  source 1..1509: mol_type = other DNA
atggacatgagggtgcccgcccagctgttgggcctgctgctgctttggctgcctggcgccaagtgccaggtgaccctgagggagtctggccccgccctggtgaagaccacccagaccctgacactgacctgcaccttcagcggcttcagcctgtccaccagcggcatgtgcgtgagctggatcaggcagccccctggaaaggccctggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggcaccctggtgaccgtgagctctggcggcggaggaagcggaggtggcggaagcggtggaggaggatctgggggcggcggcagcgatatccagatgacccagagccccagctccctgagcgccagcgtgggcgacagggtgaccatcacctgcagggcctcccagaggatcagcagccagctgaactggtaccagcagaagcccggcaaggcccctaagctcctgatctacgccgccagcagcctccagagcggcgtgcccagcaggttctccggctctggaagcggaaccgagttcaccttgaccatcagtagcctgcagcccgaggatatcgccacctactgctgccagcagacctacagcaaacccatcaccttcggccaaggcaccaggctggagatcaaggccagcaccacaacccctgcccctaggccccctacacccgctcccaccatcgctagccaacctctgtctctgagacctgaggcttgcagacctgctgccggcggcgccgtgcacaccagaggcctggacttcgcttgcgacatctacatttgggcccccttagccggaacctgcggcgttctgctgctgagcctggtgatcaccctgtactgcaaccacaggaacgtgaggagcaagaggagcaggctgctgcatagcgactacatgaacatgacccccaggcgacccggccccaccaggaagcactaccagccctatgccccacccagggacttcgccgcttacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccctaggtaatgatga

SEQ ID NO: 61 - DNA, 1569 bp, Synthetic construct
  source 1..1569: mol_type = other DNA
atggacatgagggtgcccgcccagctgctgggcctgcttctgttatggctgcccggcgccaagtgccaggtgaccctgagggagtctggacccgccctggtgaagaccacccagaccctgacactgacctgcaccttcagcggcttcagcctgtccaccagcggcatgtgcgtgagctggatcaggcagccccctggaaaggccctggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggcaccctggtgaccgtgagcagcggtggaggaggctctggcggaggagggtccggaggaggtggatctggaggcggcggcagcgacatccagatgacccagagccccagctccctgagcgccagcgtgggcgacagggtgaccatcacctgcagggcctcccagaggatcagctcccagctgaactggtaccagcagaagcccggcaaggcccccaagctgctgatctacgccgccagcagcctccagagcggcgtgcccagcaggttctccggaagcggcagcggcaccgagttcaccctgaccatttccagcctgcagcccgaggacatcgccacctactgctgccagcagacctacagcaagcccatcaccttcggccaaggcaccaggctggagatcaaggccagcctgagcaacagcatcatgtacttcagccacttcgtgcccgtgttcctgcccgccaagcccacaaccacccctgctcccagaccccctacacccgcccccaccatcgccagccaacccctttctttgagacccgaggcctgcagacccgccgccggcggagctgtgcatacaaggggcttggacttcgcttgcgacatctacatctgggcccccttagccggcacctgcggcgtgctgctgctgagcctggtgatcaccctgtactgcaaccacaggaacgtgaggagcaagaggagcaggctgctgcacagcgactacatgaacatgacccccaggaggcccggccccaccaggaagcactaccagccctatgctccacccagggattttgccgcctacaggagcagggtgaagttcagcaggagcgccgacgcccccgcctaccagcagggccagaaccagctgtacaatgagctgaacctgggcaggagggaggagtatgacgtgctggacaagaggaggggaagggaccccgagatgggcggcaagcccaggaggaagaaccctcaggagggcctgtacaacgagctccagaaggacaagatggccgaggcctacagcgagatcggcatgaagggcgagaggaggaggggcaagggccacgacggcttgtaccagggcctgagcaccgccaccaaggacacctacgacgccctgcacatgcaggccctgccccctaggtaatgataa

SEQ ID NO: 62 - DNA, 1494 bp, Synthetic construct
  source 1..1494: mol_type = other DNA
atggacatgagggtgcccgcccagctgctgggacttctgctgctgtggcttcccggcgccaagtgccaggtgcagctgcagcagagcggccccggactggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgacagtgagcagcggcggaggcggctcaggaggcggcggatctggtggaggaggaagtggtggaggtggcagcgacatccagatgacacagagcccttctagcctgagcgccagcgtgggcgacagggtgaccatcacatgcagggcctctcagaccatctggagctatctgaactggtaccagcagaggcccggaaaggcccccaacctgctgatctacgccgccagcagcctccagagcggcgtgcccagcaggttctctggcaggggcagcggcaccgacttcaccttaaccatcagcagccttcaggccgaggacttcgctacctactactgccagcagagctacagcatcccccagaccttcggccagggcaccaagctggagatcaaggccgccgccatcgaggtgatgtaccctcccccttacctggacaacgagaagtctaacggcaccatcatccacgtgaagggcaagcacctgtgccccagccccctgttccccggcccctctaagcccttctgggtcctggtggtggtgggcggcgtgctggcctgctacagcctgctggtgaccgtggccttcatcatcttctgggtgaggagcaagaggagcaggctgctgcacagcgactacatgaatatgacccccaggcgacccggccccaccaggaaacactaccagccctacgccccacccagggacttcgccgcctacaggagcagggtgaaattcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaagctctgccccctaggtaatgatga

SEQ ID NO: 63 - DNA, 1455 bp, Synthetic construct
  source 1..1455: mol_type = other DNA
atggacatgagggttcccgcccagctgctgggcctgctgctcctttggctgcccggagccaagtgccaggtgcagctgcagcagagcggacccggcctggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgaccgtgagctccggaggcggaggatctggaggtggcggaagcggcggaggtggctctggcggaggcggcagcgatatccagatgacccagagcccctccagcctgagcgccagcgtgggcgacagggtgaccatcacctgcagggcctcccagaccatctggagctacctgaactggtaccagcagaggcccggcaaggcccccaacctgctgatctacgccgccagcagcttacaaagcggcgtgcccagcaggttctctggcaggggcagcggcaccgacttcaccttaaccatcagcagcctgcaggccgaggacttcgctacctactactgccagcagagctacagcatcccccagaccttcggccagggcaccaagctggaaatcaaagctagcagccttagacccgaggcctgcagacccgccgccggcggagctgtgcatacaaggggccttgattttgcttgcgacatctacatctgggcccccttagccggcacctgcggcgtgctgctgctgagcctggtgatcaccctgtactgcaaccacaggaacgtgaggagcaagaggagcaggcttctgcacagcgactacatgaatatgacccccaggcgacccggccccaccaggaagcactatcagccctacgccccacccagggacttcgccgcctacaggagcagggtgaagttcagcaggagcgccgacgcccccgcctaccagcagggccagaaccagctgtacaatgagctgaaccttggcaggagggaggagtacgacgtgcttgataagaggaggggaagggaccccgagatgggcggcaagcccaggaggaagaacccccaggagggcctgtacaacgagctccagaaggacaagatggccgaggcctactctgagatcggcatgaagggcgagaggaggaggggcaagggccacgatggcctgtaccagggcctgagcaccgccaccaaggacacctacgacgccctgcacatgcaggccctgccccctaggtaatgatga

SEQ ID NO: 64 - DNA, 1515 bp, Synthetic construct
  source 1..1515: mol_type = other DNA
atggacatgagggtgcccgcccagctgcttggcctgttgctgctttggctgcccggcgccaagtgccaagtgcagctgcagcagagcggacccggcctggtgaagcccagccagaccctgagcctgacctgcgccatcagcggcgacagcgtgagctccaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgaccgtgagcagcggcggaggtggctctggcggaggaggatctggtgggggaggaagcggcggtggcggcagcgacatccagatgacccaaagccccagctcccttagcgccagcgtgggcgacagggtgaccatcacctgcagggcctctcagaccatctggagctatctgaactggtaccagcagaggcccggcaaagctcccaacctgttgatctacgccgccagcagcctccagagcggcgtgcctagcaggtttagtggaaggggcagcggcaccgacttcaccttaaccatcagcagccttcaagccgaggactttgccacctactactgccagcagagctacagcatcccccagaccttcggccagggcaccaagctggagatcaaggctagcaccacaacacccgcccccaggccccctacacccgctcccacaatcgcctctcagcctctgagccttaggcccgaagcttgcagacctgctgccggcggcgccgtgcataccaggggactggacttcgcttgcgacatctacatctgggcccccttagccggcacatgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaaccagctgtacaatgaactgaacctgggcaggagggaggaatacgacgttctggacaagagaaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctatgacgccctgcacatgcaggccctgccccctaggtaatgatga

SEQ ID NO: 65 - DNA, 1575 bp, Synthetic construct
  source 1..1575: mol_type = other DNA
atggacatgagggtgcccgcccagctgcttggcctgctgctcctttggctgcctggcgccaagtgccaggtgcagctgcagcagagcggacccggcctggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggcaccatggtgaccgtgagctccggcggcggaggatctggaggcggaggttctggtggcgggggaagcggaggcggcggaagcgacatccagatgacccagagccctagcagcctgagcgccagcgtgggcgacagggtgaccatcacctgcagggcctcccagaccatctggagctacctgaactggtaccagcagaggcccggcaaagctcccaacttattgatctacgccgccagcagcctccagagcggcgtgcccagcaggttctctggcaggggcagcggcaccgacttcaccttaaccatcagcagccttcaggccgaggacttcgctacctactactgccagcagagctacagcatcccccagaccttcggccaaggcaccaagctggagatcaaggccagccttagcaacagcatcatgtacttcagccacttcgtgcccgttttcctgcctgccaaacccaccacaacccctgctcccaggccccctacccccgctcctaccattgctagccaacctctgagccttaggcccgaggcctgcagacccgctgccggaggcgctgtgcacacaaggggactggacttcgcatgcgacatctacatctgggcccccttagccggcacctgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggacagaaccagctgtacaatgagctgaacctgggcaggagggaggagtacgacgtgctggacaagaggaggggaagggatcccgagatgggcggcaagcccaggaggaagaacccccaggagggcctgtacaacgagctccagaaggacaagatggccgaggcctacagcgaaatcggcatgaagggcgagaggaggaggggcaagggccacgacggcctgtaccagggcctgagcaccgccaccaaggacacatacgacgccctgcacatgcaggccctgccccctaggtaatgataa

SEQ ID NO: 66 - DNA, 8188 bp, Synthetic construct
  source 1..8188: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctcctgggcctgctgttactgtggctgcccggcgctaagtgccaggtacagctcgtggagagcggcggaggcgtggtgcagcccggcaggagcctgaggttaagctgcgccgccagcggcttcaccttcagcagctacggcatgcactgggtgagacaggcccccggcaagggcttggagtgggtggccatcatctactacgacggcagcaagaagtactacgccgacagcgtgaagggaaggttcaccatcagcagggacaacagcaagaacaccctgtacctgcagatgaacagcctgagggccgaggacaccgccgtgtactactgcgccagggagctgaccggcgacgccttcgacatctggggccagggaaccaaggtgaccgtcagcagcgggggcggaggatctggcggaggaggatcaggaggcggcggatctgggggcggcggaagcgagatcgtgctgacccagacccccagcagcctgagcgccagcctgggcgacagggtgaccatcagctgcagggcctcccagagcatcggctccagcctgcactggtaccagcagaagcccgaccagagccccaagctgctgatcaagtacgccagccagagcttcagcggcgtgcccagcaagttcatcggcagcggaagcggcaccgacttcaccttgaccatcaacagcctggaggccgaagacgccgccaccttttattgccaccagagcagcaccctgccctacaccttcggccagggcaccaagctggagatcaaggccgccgccatcgaggtgatgtaccctcccccttaccttgacaacgagaagagcaacggcaccatcatccacgtgaagggcaagcacctgtgccccagccccctgttccccggcccctctaagcccttctgggtcctggtggtggttggcggcgtgctggcctgctacagcctgctggtgaccgtggccttcatcatcttctgggtgaggagcaaaaggagcaggctgcttcacagcgactacatgaacatgacccccaggcgacccggccccaccaggaagcactaccagccctatgccccacccagggacttcgccgcctacaggtctagggtgaagttcagcaggagcgccgacgcccccgcctaccagcagggccagaaccagctgtacaatgaactgaacctgggcaggagggaggagtacgacgtgctggacaagaggaggggaagggaccccgagatgggcggcaagcccaggaggaagaacccccaggaaggcctgtacaacgagctccagaaggataaaatggccgaggcctacagcgaaatcggcatgaagggcgagaggaggaggggcaagggccacgacggcctttaccagggcctgagcaccgccaccaaggacacctacgacgccctgcacatgcaggccctgccccctaggtaatgatga

SEQ ID NO: 67 - DNA, 8149 bp, Synthetic construct
  source 1..8149: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggcctgttactcctttggctgcccggcgccaagtgccaggtgcagctggttgagagcggaggcggcgtggtgcagcccggcaggagcctgaggttaagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtacctgcagatgaacagcctgagagccgaggacaccgccgtgtactactgcgccagggagctgaccggcgacgccttcgacatctggggccagggaaccaaggtgaccgtgagcagcggcggcggaggatctggcggaggaggctcaggaggaggtggaagcgggggcggcggctctgagatcgtgctgacccagacccccagctctctgagcgccagcctgggcgacagggtgaccatcagctgcagggcctcccagagcatcggctctagcctgcactggtaccagcagaagcccgaccagagccccaagctgctgatcaagtacgccagccagagcttcagcggcgtgcccagcaagttcatcggcagcgggagcggcaccgacttcaccttgaccatcaacagcctggaggccgaggacgccgccaccttctactgccaccagagcagcaccctgccctacaccttcggccagggcaccaagctggagatcaaggccagcagcttaagacccgaagcctgcaggcccgccgccggaggagctgttcacaccaggggcttggattttgcttgcgatatctacatctgggcccccttagccggcacctgcggcgtgctgctgctgtctctggtgatcaccttatactgtaaccacaggaacgtgaggagcaagagaagcaggctgctgcacagcgactacatgaacatgacccctaggaggcccggccccaccaggaagcactaccagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgcttgacaagaggaggggaagggaccccgaaatgggcggcaagcccaggaggaaaaacccccaagagggcctgtacaacgagctccagaaggacaagatggccgaggcctacagcgagatcggcatgaagggcgagaggaggaggggcaagggccacgacggcctgtaccagggccttagcaccgccaccaaggacacctacgacgccctgcacatgcaggccctgccccctaggtaatgatga

SEQ ID NO: 68 - DNA, 8209 bp, Synthetic construct
  source 1..8209: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggactgctgctcctttggctgcctggcgccaagtgccaggtgcagctggtggagagcggcggaggagtggtgcagcccggcaggagcctgagactgagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtatctgcagatgaacagcctgagggccgaggataccgccgtgtactactgcgccagggagctgaccggcgacgccttcgacatctggggccagggaaccaaggtgaccgtgagcagcggaggcggaggatctggaggtggcggtagcggaggaggcggaagcggcggtggcggcagcgaaattgtgctgacccagacccccagcagcctcagcgccagcctgggcgacagggtgaccatcagctgcagggcctcccagagcatcggctccagcctgcactggtaccagcagaagcccgaccagagccccaagctgctgatcaagtacgccagccaaagcttcagcggcgtgcccagcaagttcatcggaagcggatccggaaccgacttcaccttaaccatcaacagcctggaggccgaggacgccgccaccttctactgtcaccagagcagcaccctgccctacacctttggccagggcaccaagctggaaatcaaagccagcaccaccacccccgctcctaggcctcccacacccgcccccacaattgctagccagcctttgagcctgaggcctgaagcttgtagacccgctgccggaggcgccgtgcacaccaggggcctggacttcgcttgcgacatctacatctgggcccccttggccggcacctgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctataggagcagggtgaagttcagcaggagcgccgacgcccccgcctaccagcagggccagaaccagctgtacaatgagctgaacctgggcaggagggaggagtacgacgtgttagacaagaggaggggaagggatcccgagatgggcggcaagcccaggaggaagaacccccaggaaggcctgtacaacgagctccagaaggacaagatggctgaggcctacagcgaaatcggaatgaagggcgagaggaggaggggcaagggccacgacggcctgtatcagggcctgagcaccgccaccaaggacacctacgacgccctgcacatgcaggccctgccccctaggtaatgatga

SEQ ID NO: 69 - DNA, 8269 bp, Synthetic construct
  source 1..8269: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccaactgcttggcctgctgctcctttggctgcccggcgccaagtgccaggtgcagctggtggagtctggcggaggcgtggtgcagcccggaaggagcctgagactgagctgcgccgccagcggcttcaccttcagcagctacggcatgcactgggtgaggcaggcccccggcaagggcttggagtgggtggccatcatctactacgacggcagcaagaagtactacgccgacagcgtgaagggcaggttcaccatcagcagggacaacagcaagaacaccctgtatctgcagatgaacagcctgagggccgaggataccgccgtgtactactgcgccagggagctgaccggcgacgccttcgacatctggggccagggaaccaaggtgaccgtgagcagcggcggaggaggatctggaggcggcggatcaggaggtgggggaagcgggggaggcggcagcgaaatcgtgctgacccagacccccagcagcctcagcgccagcctgggcgacagggtgaccatcagctgcagggcctcccagagcatcggctccagcctgcactggtaccagcagaagcccgaccagagccccaagctgctgatcaagtacgccagccaaagcttcagcggcgtgcccagcaagttcatcggcagcggaagcggcaccgacttcaccttgaccatcaacagcctggaggccgaggacgccgccaccttctactgccaccagagcagcaccctgccctacaccttcggccagggcaccaagctggagatcaaggccagcctgagcaacagcatcatgtacttcagccacttcgtgcccgtgttcctgcccgccaagcctaccacaacacccgcccccaggcccccaacacctgctcccacaatcgccagccagcccttatccttaaggcccgaggcctgcagacccgctgccggaggcgctgtgcataccaggggactggacttcgcttgcgacatctacatctgggcccccttagccggaacatgcggcgtgctgctgctgagcctggtgatcaccctgtactgcaaccacaggaacgtgaggagcaagaggagcaggctgctgcacagcgactacatgaacatgacccccaggcgacccggccctaccaggaaacattatcagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggaaagcccaggaggaagaacccccaggagggcctgtacaacgagctccagaaggacaagatggccgaggcctatagcgagatcggcatgaagggcgagaggaggaggggcaagggccacgacggcctgtaccaaggcctgagcaccgccaccaaggacacctacgacgctcttcacatgcaggccctgccccctaggtaatgatga

SEQ ID NO: 70 - DNA, 8188 bp, Synthetic construct
  source 1..8188: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcctgcccagctgctgggcctgctgctgctgtggcttcctggcgccaagtgccaggtgcagctggtgcagagcggcgccgaggtgaagaagcctggcgcctccgtgaaggtgagctgcaaggccagcggctacaccttcaccggctactacatccactgggtgagacaggcccccggccagggactggagtggatgggctggatcaaccccaacagcggcggcaccaactacgcccagaagttccagggcagggtgacaatgaccagggacaccagcatcggcaccgcctacatggagctgagcaggctcaggagcgacgacaccgccatctactactgcaccagggaggacacctggaactacatcgactactggggccagggaaccctggtgacagtgagcagcggaggtggcggaagcggcggcggaggatcagggggaggtggaagtggcggaggaggcagcgacatccagatgacccagagccccagctccctgagcgccagcgtgggcgacagggtgaccatcacctgcagggcctcccagagcatcagcagccacctgaactggtaccagcagaagcccggcaaggcccccaagctgctgatctacgccgccagcagcctccacagcggcgtgcccagcaggttctctggctctggcagcggcaccgacttcaccttaacaatttctagcctgcaacccgaggacttcgctacctactgctgccagcagagctacagcacccccagaacctttggccagggcaccaaggtggagatcaaggccgccgccatcgaggtgatgtaccctcccccttacctggacaacgagaagagcaacggcaccatcatccacgtgaagggcaagcacctgtgccccagccccctgttccccggcccctctaagcccttctgggtcctggtggtggtgggcggcgtgctggcctgctatagcctgctggtgaccgtggccttcatcatcttctgggtgaggagcaagaggagcaggctgctgcacagcgattacatgaacatgacccccaggcgacccggccctaccaggaagcactaccagccctacgccccacccagggacttcgccgcctacaggagcagggtgaagttcagcaggagcgccgacgcccccgcttaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaagacaagatggccgaggcctacagcgagatcggcatgaagggcgagaggaggaggggcaagggccacgacggcctgtaccagggcctgagcaccgccaccaaggatacctacgatgccctgcacatgcaggccctgcctcctaggtaatgatga

SEQ ID NO: 71 - DNA, 8149 bp, Synthetic construct
  source 1..8149: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggcctgctgttgctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagggtgacaatgaccagggacaccagcatcggcaccgcctacatggagctgagcaggctcaggagcgacgacaccgccatctactactgcaccagggaggacacctggaactacatcgactactggggccagggaaccctggtgaccgtgagcagcggagggggaggaagcggcggaggaggcagtggaggaggaggatccggaggcggcggaagcgacatccagatgacccagagccccagctccctgagcgccagcgtgggcgacagggtgaccatcacctgcagggcctctcagagcatcagctctcacctgaattggtaccagcagaagcccggcaaggcccccaagctgctgatctacgccgccagcagcttacacagcggcgtgcccagcaggttttctggcagcggaagcggcaccgacttcaccttaacaatctccagcctgcagcccgaggactttgctacctactgctgccagcagagctacagcacccccagaaccttcggccagggcaccaaggtggaaatcaaggcctcttctctgagacccgaggcttgcagacccgccgctggaggcgccgttcataccaggggcctggacttcgcttgcgacatctacatctgggcccccttggccggcacctgcggcgtgctgctgctgagcctggtgatcaccctgtactgcaaccacaggaacgtgaggagcaagaggagcaggctgctgcatagcgactacatgaacatgacccccaggcgacccggccccaccaggaagcactaccagccctacgccccacccagggacttcgccgcctacaggagcagggtgaagttcagcaggagcgctgatgcccccgcctaccagcagggccagaaccagctgtacaatgagctgaatctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggagaggcaagggccacgacggcctgtaccagggcctgagcaccgccaccaaagacacctacgacgccctgcacatgcaggccctgccccctaggtaatgatga

SEQ ID NO: 72 - DNA, 8209 bp, Synthetic construct
  source 1..8209: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggactgctgctcctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagagtgaccatgaccagggacaccagcatcggcaccgcctacatggagctgagcaggctcaggagcgacgacaccgccatctactactgcaccagggaggacacctggaactacatcgactactggggccagggcaccctggtgaccgtgagcagcggaggaggcggatctggcggcggaggatcaggagggggaggaagcggaggcggaggcagcgacatccagatgacccagagccccagctccctgagcgccagcgtgggcgacagggttaccattacctgcagggcctctcagtctatcagctctcatctgaactggtaccagcagaagcccggcaaggcccctaagctgctgatctatgccgccagcagcctccacagcggcgtgcctagcaggttctctggcagcggaagtggcaccgacttcaccttaaccatcagctcactgcagcccgaggacttcgctacctactgctgccagcagagctacagcacccccagaaccttcggacagggcaccaaggtggagatcaaggcctccacaacaacacccgctcccaggccccctacccccgcccccacaattgccagccaacccctcagcctgagacccgaagcctgtagacctgccgcagggggagccgtgcacaccaggggcctggacttcgcatgcgacatctacatctgggcccccttagccggcacctgcggcgtgctgctgctgagccttgtgatcaccctgtactgcaaccacaggaacgtgaggagcaagaggagcaggctgctgcacagcgactacatgaacatgacccccaggcgacccggccccaccaggaagcattaccagccctacgccccacccagggacttcgccgcctacaggagcagggtgaagttcagcaggagcgccgacgcccctgcctaccagcagggccagaaccagctgtacaatgagctgaacctgggcaggagggaggagtatgacgtgctggacaagaggaggggaagggaccccgagatgggcggcaagcccaggaggaagaacccccaggagggcctgtataacgagctccagaaggacaagatggccgaagcctacagcgagatcggcatgaagggcgagaggaggaggggcaagggccatgacggcctgtaccagggcctgagcaccgccaccaaggacacctacgacgccctgcacatgcaggccctgccccctaggtaatgatga

SEQ ID NO: 73 - DNA, 8269 bp, Synthetic construct
  source 1..8269: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggactgctgctcctttggctgcccggcgctaagtgccaggtgcagctggtgcagagcggcgccgaggtgaagaagcctggcgcctccgtgaaggtgagctgcaaggccagcggctacaccttcaccggctactacatccactgggtgaggcaggcccccggccagggactggagtggatgggctggatcaaccccaacagcggcggcaccaactacgcccagaagttccagggcagagtgaccatgaccagggacaccagcatcggcaccgcctacatggagctgagcaggctcaggagcgacgacaccgccatctactactgcaccagggaggacacctggaactacatcgactactggggccagggcaccctggtgaccgtgagcagcggaggaggcggatctggcggcggaggatcaggagggggaggaagcggaggcggaggcagcgacatccagatgacccagagccccagctccctgagcgccagcgtgggcgacagggttaccattacctgcagggcctctcagtctatcagctctcatctgaactggtaccagcagaagcccggcaaggcccctaagctgctgatctatgccgccagcagcctccacagcggcgtgcctagcaggttctctggcagcggaagtggcaccgacttcaccttaaccatcagctcactgcagcccgaggacttcgctacctactgctgccagcagagctacagcacccccagaaccttcggacagggcaccaaggtggagatcaaggcctccacaacaactagcatcatgtacttcagccatttcgtgcccgtgtttcttcccgctaaacccaccactacacccgctcccaggccccctacccccgcccccacaattgccagccaacccctcagcctgagacccgaagcctgtagacctgccgcagggggagccgtgcacaccaggggcctggacttcgcatgcgacatctacatctgggcccccttagccggcacctgcggcgtgctgctgctgagccttgtgatcaccctgtactgcaaccacaggaacgtgaggagcaagaggagcaggctgctgcacagcgactacatgaacatgacccccaggcgacccggccccaccaggaagcattaccagccctacgccccacccagggacttcgccgcctacaggagcagggtgaagttcagcaggagcgccgacgcccctgcctaccagcagggccagaaccagctgtacaatgagctgaacctgggcaggagggaggagtatgacgtgctggacaagaggaggggaagggaccccgagatgggcggcaagcccaggaggaagaacccccaggagggcctgtataacgagctccagaaggacaagatggccgaagcctacagcgagatcggcatgaagggcgagaggaggaggggcaagggccatgacggcctgtaccagggcctgagcaccgccaccaaggacacctacgacgccctgcacatgcaggccctgccccctaggtaatgatga

SEQ ID NO: 74 - DNA, 8200 bp, Synthetic construct
  source 1..8200: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcctgcccagttgttgggcctgctgctgctgtggctgcccggcgccaagtgccaggttaccctgagggagtctggccccgccctggtgaagcccacccagaccttaaccctgacctgcaccttcagcggcatcagcctgaccaccagcggcatgtgcgtgagctggatcaggcagcccccaggcaaggccctggagtggctggccctgatcgactgggacggcgacaactactacagcaccagcctgaagaccaggctgaccatctccaaggacaccagcaagaaccaggtggtgctgaccatggccagcatggaccccgtggacaccgccacatactactgcgccaggatgggctacacctacggctacgacgctttcgacatctggggccagggcaccatggtgacagtgagcagcggcggtggaggatctggcggaggcggaagcggaggaggcggatctgggggcggaggaagcgagattgtgctgacccagacccccagcagcctcagcgccagcctgggcgacagggtgaccatcagctgcagggcctctcagagcatcggcagcctgttacactggtaccagcagaagcccgaccagagccccaagctgctgatcaagttcgccagccagagcctgagcggcgtgcccagcaggttctctggctctggcagcggcaccgacttcaccttaaccatcaacagcctggaggccgaagacgccgccacctactactgccaccagagcaggagcctgccctttaccttcggccccggcaccaaggttgacatcaacgccgccgccatcgaggtgatgtaccctcccccttacctggacaacgagaagagcaacggcaccatcatccacgtgaagggcaagcacctgtgccccagccccctgttccccggcccctctaagcccttctgggttctggtggtggtgggcggcgtgctggcctgctacagcctgctggtgaccgtggccttcatcatcttctgggtgaggagtaagaggagcaggctgctgcacagcgactacatgaacatgacccccaggcgacccggccccaccaggaagcactaccagccctacgcccctccaagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaaaatggccgaagcctacagcgagatcggcatgaagggcgagaggaggaggggcaaaggccacgacggcctgtaccagggcctgagcaccgccaccaaagacacctacgacgccctgcacatgcaggccctgccccctaggtaatgatga

SEQ ID NO: 75 - DNA, 8161 bp, Synthetic construct
  source 1..8161: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggcctgctgttactttggctgcccggcgccaagtgccaagtgaccctgagggagtctggccccgccctggtgaagcccacccagaccctcaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctccaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagcagcggcggaggaggatctggaggcggaggctctggtggaggaggcagcgggggcggaggaagcgagattgtgctgacacagacccccagctccctgagcgccagcctgggcgacagggtgaccatcagctgcagggcctcccagagcatcggcagcctgttgcactggtaccagcagaagcccgatcagagccccaaactgctgatcaagttcgccagccagagcctgagcggcgtgcccagcaggttctctggaagcggctctggcaccgacttcaccttaaccatcaacagcctggaggccgaggacgccgccacctactactgccaccaaagcaggagcctgccctttaccttcggccccggcaccaaggtggacatcaacgccagcagcctcaggcccgaagcttgtaggcctgctgccggcggcgctgttcacaccaggggcttagactttgcttgcgacatctacatctgggcccccttagccggaacctgcggcgtgctgctgctgagcctggtgatcaccctgtactgcaaccacaggaatgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaatcagctgtacaatgagctgaacctgggcaggagggaggagtacgacgtgctggacaagaggaggggaagggaccctgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcttaagcaccgccaccaaggatacctacgacgccctgcacatgcaggccctgccccctaggtaatgatga

SEQ ID NO: 76 - DNA, 8221 bp, Synthetic construct
  source 1..8221: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagcttctgggcctgctgctcttgtggctgcctggcgccaagtgccaggtgaccctgagggagtctggccccgccctggtgaagcccacccagaccttgaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctctaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagctctggcgggggaggaagcggcggcggaggatctggtggaggtgggtctggaggcggcggcagcgaaatcgtgctgacccagacccccagcagcctcagcgccagcctgggcgacagggtgaccatcagctgcagggcctcccagagcatcggcagcctgttgcactggtaccagcagaagcccgaccagagccccaagctgctgatcaagttcgccagccagagcctgagcggcgtgcccagcaggttctctggaagcggctctggaaccgacttcaccttaaccatcaacagcctggaggccgaagacgccgccacctattactgccaccagagcaggagcctgcccttcaccttcggacccggaaccaaggtggacatcaacgccagcacaaccacacccgcccctaggccccctacccccgcccccacaatcgctagccaacccctgagccttaggcccgaagcctgtagacctgccgccggaggcgccgtgcataccaggggcctggacttcgcttgtgacatctacatctgggcccccttagccggaacctgcggcgtgctgctgctgagcctggtgatcaccctgtactgcaaccacaggaacgtgaggagcaaaaggagcaggctgctgcacagcgactacatgaacatgacccccaggaggcccggccccaccaggaagcactaccagccctacgccccacccagggacttcgccgcctacaggagcagggtgaagtttagcaggagcgccgacgcccccgcctaccagcagggccagaatcagctgtacaatgaattaaacctgggcaggagggaggagtacgacgtgctggataagaggaggggaagggaccccgagatgggcggcaagcccaggaggaagaaccctcaggagggactgtacaacgagctccagaaggataagatggccgaggcctacagcgagatcggcatgaagggcgagaggaggaggggcaagggccacgacggcctgtaccaaggcctgagcaccgccaccaaggacacctacgacgccctgcacatgcaggccctgccccctagataatgatga

SEQ ID NO: 77 - DNA, 8281 bp, Synthetic construct
  source 1..8281: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccaactgctgggcctgctgctcctttggctgcctggcgccaagtgccaggtgaccctgagggagtctggccccgccctggtgaagcccacccagaccttgaccctgacctgcaccttcagcggcatcagcctgaccaccagcggcatgtgcgtgagctggatcaggcagccccctggcaaggccctggagtggctggccctgatcgactgggacggcgacaactactacagcaccagcctgaagaccaggctgaccatctctaaggacaccagcaagaaccaggtggtgctgaccatggccagcatggaccccgtggacaccgccacatactactgcgccaggatgggctacacctacggctacgacgctttcgacatctggggccagggcaccatggtgaccgtgagcagcggcggaggcggttctggtggcggaggctcaggaggcggaggatctggaggcggcggcagcgaaatcgtgctgacccagacccccagcagcctcagcgccagcctcggcgacagggtgaccatcagctgcagggcctcccagagcatcggcagcctgctgcactggtaccagcagaagcccgaccagagccccaagctgctgatcaagttcgccagccagagcctgagcggcgtgcccagcaggttctctggcagcggctctggcaccgacttcaccttaaccatcaacagcctggaggccgaggacgccgccacctactactgccatcaaagcaggagcctgcccttcaccttcggccccggcacaaaagtggacatcaacgccagcctgagcaacagcatcatgtactttagccattttgtgcccgtgttcctgcctgccaagcccacaacaacccccgcccccaggcctcctacccctgcccctacaatcgctagccaacccctgtctctgagacccgaggcctgcagacctgctgccggaggcgccgtgcacaccaggggactggacttcgcttgcgacatctatatctgggcccccttagccggcacctgcggcgtgctgctgctgagcctggtgattaccctgtactgcaaccacaggaacgtgaggagcaagaggagcagactgctgcacagcgactacatgaacatgacccccaggcgacccggccccaccaggaagcactaccagccctacgccccacccagggacttcgccgcctacaggagcagggtgaagttcagcaggtcagccgacgcccccgcctaccaacagggccagaaccagctgtacaatgagctgaacctgggcaggagggaggagtacgacgtgctggacaagaggaggggaagggatcctgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgctaccaaggacacctacgacgccctgcacatgcaggccctgccccctaggtaatgatga

SEQ ID NO: 78 - DNA, 8200 bp, Synthetic construct
  source 1..8200: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggcctgttactgctgtggctgcccggcgctaagtgccaggtgaccctgagggagtctggccccgccctggtgaagaccacccagaccctgacactgacctgcaccttcagcggcttcagcctgtccaccagcggcatgtgcgtgagctggatcaggcagcccccaggcaaggccttggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggaaccctggtcaccgtgagcagcggaggcggtggatctggaggcggaggaagcggcggaggaggatccggaggcggcggcagcgatatccagatgacccagagccccagctccctgagcgcctctgtgggcgacagggtgaccatcacctgcagggcctctcagaggatcagctctcagctgaactggtaccagcagaagcccggcaaggcccccaagctgctgatctacgccgccagcagcctccagagcggcgtgccctctaggttctctggctctggaagcggcaccgagttcaccctgaccattagcagcttgcagcccgaggacatcgccacctactgctgccagcagacctacagcaagcctatcaccttcggccagggcaccaggctggagatcaaggccgccgccatcgaggtgatgtaccctcccccttacctggacaacgagaagagcaacggcaccatcatccacgtgaagggcaagcacctgtgccccagccccctgttccccggcccctctaagcccttctgggtcctggtggtggtgggcggcgtgctggcctgctacagcctgctggtgaccgtggccttcatcatcttctgggtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaggccttaccccctaggtaatgatga

SEQ ID NO: 79 - DNA, 8161 bp, Synthetic construct
  source 1..8161: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccaactgctgggcctgttgctgctttggctgcccggcgccaagtgccaggtgaccctgagggagtctggacccgccctggtgaagaccacccagaccctgacactgacctgcaccttcagcggcttcagcctgtccaccagcggcatgtgcgtgagctggatcaggcagccccctggcaaggccttggagtggctggccctgatcgactgggacgacgacaagtactacagcaccagcctgaagaccaggctgaccatcagcaaggacaccagcaagagccaggtggtgctgaccatgaccagcatggaccccgtggacaccgccacatactactgcgccaggatcaggtacatctacggctacgactacttcgactactggggccagggcaccctggtgaccgtgagcagcggaggcggaggatctggcggagggggatctggaggtggaggctctggcggcggcggcagcgatatccagatgacccagagccccagctccctgagcgccagcgtgggcgacagggtgaccatcacctgcagggcctcccagaggatcagctcccagctgaactggtaccagcagaagcccggcaaggcccccaagctgctgatctacgccgccagcagccttcagagcggcgtgcccagcaggttctccggcagcgggagcggcaccgagttcaccctgaccattagctcactgcagcccgaggacatcgccacctactgctgccagcagacctacagcaagcccatcaccttcgggcagggcaccaggctggagatcaaggcctcttctttgagacccgaggcttgtaggcccgcagccggcggcgccgtgcacacaaggggactggatttcgcttgcgacatctatatctgggcccccttagccggcacctgcggcgtgctgctgctgagcctggtgatcaccctgtactgcaaccacaggaacgtgaggagcaagaggagcaggctgctgcacagcgactacatgaacatgacacccaggaggcccggccctaccaggaagcactaccagccctacgccccacccagggacttcgccgcttataggagcagggtgaagttcagcaggagcgccgacgctcccgcctaccagcagggccagaaccagctgtacaatgagctgaacctgggcaggagggaggagtacgacgtgctggacaagagaaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaagacacctacgacgccctgcacatgcaggccctgccccctaggtaatgatga

SEQ ID NO: 80 - DNA, 8221 bp, Synthetic construct
  source 1..8221: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgttgggcctgctgctgctttggctgcctggcgccaagtgccaggtgaccctgagggagtctggccccgccctggtgaagaccacccagaccctgacactgacctgcaccttcagcggcttcagcctgtccaccagcggcatgtgcgtgagctggatcaggcagccccctggaaaggccctggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggcaccctggtgaccgtgagctctggcggcggaggaagcggaggtggcggaagcggtggaggaggatctgggggcggcggcagcgatatccagatgacccagagccccagctccctgagcgccagcgtgggcgacagggtgaccatcacctgcagggcctcccagaggatcagcagccagctgaactggtaccagcagaagcccggcaaggcccctaagctcctgatctacgccgccagcagcctccagagcggcgtgcccagcaggttctccggctctggaagcggaaccgagttcaccttgaccatcagtagcctgcagcccgaggatatcgccacctactgctgccagcagacctacagcaaacccatcaccttcggccaaggcaccaggctggagatcaaggccagcaccacaacccctgcccctaggccccctacacccgctcccaccatcgctagccaacctctgtctctgagacctgaggcttgcagacctgctgccggcggcgccgtgcacaccagaggcctggacttcgcttgcgacatctacatttgggcccccttagccggaacctgcggcgttctgctgctgagcctggtgatcaccctgtactgcaaccacaggaacgtgaggagcaagaggagcaggctgctgcatagcgactacatgaacatgacccccaggcgacccggccccaccaggaagcactaccagccctatgccccacccagggacttcgccgcttacaggagcagggtgaagttcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccctaggtaatgatga

SEQ ID NO: 81 - DNA, 8281 bp, Synthetic construct
  source 1..8281: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggcctgcttctgttatggctgcccggcgccaagtgccaggtgaccctgagggagtctggacccgccctggtgaagaccacccagaccctgacactgacctgcaccttcagcggcttcagcctgtccaccagcggcatgtgcgtgagctggatcaggcagccccctggaaaggccctggagtggctggccctgatcgactgggacgacgacaagtactacagcaccagcctgaagaccaggctgaccatctctaaggacaccagcaagagccaggtggtgctgaccatgaccagcatggaccccgtggacaccgccacatactactgcgccaggatcaggtacatctacggctacgactacttcgactactggggccagggcaccctggtgaccgtgagcagcggtggaggaggctctggcggaggagggtccggaggaggtggatctggaggcggcggcagcgacatccagatgacccagagccccagctccctgagcgccagcgtgggcgacagggtgaccatcacctgcagggcctcccagaggatcagctcccagctgaactggtaccagcagaagcccggcaaggcccccaagctgctgatctacgccgccagcagcctccagagcggcgtgcccagcaggttctccggaagcggcagcggcaccgagttcaccctgaccatttccagcctgcagcccgaggacatcgccacctactgctgccagcagacctacagcaagcccatcaccttcggccaaggcaccaggctggagatcaaggccagcctgagcaacagcatcatgtacttcagccacttcgtgcccgtgttcctgcccgccaagcccacaaccacccctgctcccagaccccctacacccgcccccaccatcgccagccaacccctttctttgagacccgaggcctgcagacccgccgccggcggagctgtgcatacaaggggcttggacttcgcttgcgacatctacatctgggcccccttagccggcacctgcggcgtgctgctgctgagcctggtgatcaccctgtactgcaaccacaggaacgtgaggagcaagaggagcaggctgctgcacagcgactacatgaacatgacccccaggaggcccggccccaccaggaagcactaccagccctatgctccacccagggattttgccgcctacaggagcagggtgaagttcagcaggagcgccgacgcccccgcctaccagcagggccagaaccagctgtacaatgagctgaacctgggcaggagggaggagtatgacgtgctggacaagaggaggggaagggaccccgagatgggcggcaagcccaggaggaagaaccctcaggagggcctgtacaacgagctccagaaggacaagatggccgaggcctacagcgagatcggcatgaagggcgagaggaggaggggcaagggccacgacggcttgtaccagggcctgagcaccgccaccaaggacacctacgacgccctgcacatgcaggccctgccccctaggtaatgataa

SEQ ID NO: 82 - DNA, 8206 bp, Synthetic construct
  source 1..8206: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgctgggacttctgctgctgtggcttcccggcgccaagtgccaggtgcagctgcagcagagcggccccggactggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgacagtgagcagcggcggaggcggctcaggaggcggcggatctggtggaggaggaagtggtggaggtggcagcgacatccagatgacacagagcccttctagcctgagcgccagcgtgggcgacagggtgaccatcacatgcagggcctctcagaccatctggagctatctgaactggtaccagcagaggcccggaaaggcccccaacctgctgatctacgccgccagcagcctccagagcggcgtgcccagcaggttctctggcaggggcagcggcaccgacttcaccttaaccatcagcagccttcaggccgaggacttcgctacctactactgccagcagagctacagcatcccccagaccttcggccagggcaccaagctggagatcaaggccgccgccatcgaggtgatgtaccctcccccttacctggacaacgagaagtctaacggcaccatcatccacgtgaagggcaagcacctgtgccccagccccctgttccccggcccctctaagcccttctgggtcctggtggtggtgggcggcgtgctggcctgctacagcctgctggtgaccgtggccttcatcatcttctgggtgaggagcaagaggagcaggctgctgcacagcgactacatgaatatgacccccaggcgacccggccccaccaggaaacactaccagccctacgccccacccagggacttcgccgcctacaggagcagggtgaaattcagcaggagcgccgacgcccccgcctaccagcagggccagaaccagctgtacaatgagctgaacctgggcaggagggaggagtacgacgtgctggacaagaggaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctacgacgccctgcacatgcaagctctgccccctaggtaatgatga

SEQ ID NO: 83 - DNA, 8167 bp, Synthetic construct
  source 1..8167: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggttcccgcccagctgctgggcctgctgctcctttggctgcccggagccaagtgccaggtgcagctgcagcagagcggacccggcctggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgaccgtgagctccggaggcggaggatctggaggtggcggaagcggcggaggtggctctggcggaggcggcagcgatatccagatgacccagagcccctccagcctgagcgccagcgtgggcgacagggtgaccatcacctgcagggcctcccagaccatctggagctacctgaactggtaccagcagaggcccggcaaggcccccaacctgctgatctacgccgccagcagcttacaaagcggcgtgcccagcaggttctctggcaggggcagcggcaccgacttcaccttaaccatcagcagcctgcaggccgaggacttcgctacctactactgccagcagagctacagcatcccccagaccttcggccagggcaccaagctggaaatcaaagctagcagccttagacccgaggcctgcagacccgccgccggcggagctgtgcatacaaggggccttgattttgcttgcgacatctacatctgggcccccttagccggcacctgcggcgtgctgctgctgagcctggtgatcaccctgtactgcaaccacaggaacgtgaggagcaagaggagcaggcttctgcacagcgactacatgaatatgacccccaggcgacccggccccaccaggaagcactatcagccctacgccccacccagggacttcgccgcctacaggagcagggtgaagttcagcaggagcgccgacgcccccgcctaccagcagggccagaaccagctgtacaatgagctgaaccttggcaggagggaggagtacgacgtgcttgataagaggaggggaagggaccccgagatgggcggcaagcccaggaggaagaacccccaggagggcctgtacaacgagctccagaaggacaagatggccgaggcctactctgagatcggcatgaagggcgagaggaggaggggcaagggccacgatggcctgtaccagggcctgagcaccgccaccaaggacacctacgacgccctgcacatgcaggccctgccccctaggtaatgatga

SEQ ID NO: 84 - DNA, 8227 bp, Synthetic construct
  source 1..8227: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgcttggcctgttgctgctttggctgcccggcgccaagtgccaagtgcagctgcagcagagcggacccggcctggtgaagcccagccagaccctgagcctgacctgcgccatcagcggcgacagcgtgagctccaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggaaccatggtgaccgtgagcagcggcggaggtggctctggcggaggaggatctggtgggggaggaagcggcggtggcggcagcgacatccagatgacccaaagccccagctcccttagcgccagcgtgggcgacagggtgaccatcacctgcagggcctctcagaccatctggagctatctgaactggtaccagcagaggcccggcaaagctcccaacctgttgatctacgccgccagcagcctccagagcggcgtgcctagcaggtttagtggaaggggcagcggcaccgacttcaccttaaccatcagcagccttcaagccgaggactttgccacctactactgccagcagagctacagcatcccccagaccttcggccagggcaccaagctggagatcaaggctagcaccacaacacccgcccccaggccccctacacccgctcccacaatcgcctctcagcctctgagccttaggcccgaagcttgcagacctgctgccggcggcgccgtgcataccaggggactggacttcgcttgcgacatctacatctgggcccccttagccggcacatgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggccagaaccagctgtacaatgaactgaacctgggcaggagggaggaatacgacgttctggacaagagaaggggaagggaccccgagatgggcggcaagcccaggaggaagaacccccaggagggcctgtacaacgagctccagaaggacaagatggccgaggcctacagcgagatcggcatgaagggcgagaggaggaggggcaagggccacgacggcctgtaccagggcctgagcaccgccaccaaggacacctatgacgccctgcacatgcaggccctgccccctaggtaatgatga

SEQ ID NO: 85 - DNA, 8287 bp, Synthetic construct
  source 1..8287: mol_type = other DNA
tctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaaggcggccgctgccaagcttccgagctctcgaattcatggacatgagggtgcccgcccagctgcttggcctgctgctcctttggctgcctggcgccaagtgccaggtgcagctgcagcagagcggacccggcctggtgaagcccagccagaccctgagcctgacctgcgccatcagcggcgacagcgtgagcagcaacagcgccgcctggaactggatcaggcagagccccagcaggggcctggagtggctgggcagaacctactacaggtccaagtggtacaacgactacgccgtgagcgtgaagagcaggatcaccatcaaccccgacaccagcaagaaccagttctcactgcagctgaacagcgtgacccccgaggacaccgccgtgtactactgcgccagggaggtgaccggcgacctggaggacgccttcgacatctggggccagggcaccatggtgaccgtgagctccggcggcggaggatctggaggcggaggttctggtggcgggggaagcggaggcggcggaagcgacatccagatgacccagagccctagcagcctgagcgccagcgtgggcgacagggtgaccatcacctgcagggcctcccagaccatctggagctacctgaactggtaccagcagaggcccggcaaagctcccaacttattgatctacgccgccagcagcctccagagcggcgtgcccagcaggttctctggcaggggcagcggcaccgacttcaccttaaccatcagcagccttcaggccgaggacttcgctacctactactgccagcagagctacagcatcccccagaccttcggccaaggcaccaagctggagatcaaggccagccttagcaacagcatcatgtacttcagccacttcgtgcccgttttcctgcctgccaaacccaccacaacccctgctcccaggccccctacccccgctcctaccattgctagccaacctctgagccttaggcccgaggcctgcagacccgctgccggaggcgctgtgcacacaaggggactggacttcgcatgcgacatctacatctgggcccccttagccggcacctgcggcgtgctgctgctgagcctggtgatcaccctgtactgcaaccacaggaacgtgaggagcaagaggagcaggctgctgcacagcgactacatgaacatgacccccaggcgacccggccccaccaggaagcactaccagccctacgccccacccagggacttcgccgcctacaggagcagggtgaagttcagcaggagcgccgacgcccccgcctaccagcagggacagaaccagctgtacaatgagctgaacctgggcaggagggaggagtacgacgtgctggacaagaggaggggaagggatcccgagatgggcggcaagcccaggaggaagaacccccaggagggcctgtacaacgagctccagaaggacaagatggccgaggcctacagcgaaatcggcatgaagggcgagaggaggaggggcaagggccacgacggcctgtaccagggcctgagcaccgccaccaaggacacatacgacgccctgcacatgcaggccctgccccctaggtaatgataa

SEQ ID NO: 86 - RNA, 1470 nt, Synthetic construct
  source 1..1470: mol_type = other RNA
atggagaccgacaccctcctgctgtgggtgctgctgctttgggtgcccgggagcacggggcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 87 - RNA, 1470 nt, Synthetic construct
  source 1..1470: mol_type = other RNA
atggagaccgacaccctcctgctgtgggtgctgctgctttgggtgcccgggagcacggggcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 88 - RNA, 1491 nt, Synthetic construct
  source 1..1491: mol_type = other RNA
atggagaccgacaccctcctgctgtgggtgctgctgctttgggtgcccgggagcacggggcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcgagcaccaccacccccgcgccccggccgccgacccccgcccccacaatcgcctcacagccactgagcctgcgccccgaggcctgcaggcccgccgcaggcggcgccgtgcacacgcgtgggctcgacttcgcctgtgacatatacatctgggcccccctggccggcacctgcggggtcctgctgctgagcctggtgatcaccctgtattgcaaccaccggaacgtgcgctccaaacgcagccggctgctgcacagcgactacatgaacatgaccccgcgcaggcccggcccgacccgaaagcactaccagccatacgcccccccccgggacttcgccgcctaccggtcaagggttaagttctcacggtcggccgacgcccccgcctaccagcagggccagaaccagctgtacaatgagctgaacctgggccggagggaggagtacgacgtactggataaaaggcgcggcagagaccccgagatgggggggaaacccaggcgtaagaacccccaggagggcctgtacaacgaactgcaaaaggacaagatggccgaggcctacagcgaaataggcatgaagggcgagcgtaggagggggaagggccacgacggcctgtatcaaggcctgtcgaccgccaccaaagacacctacgacgccctgcacatgcaggccctgccccccaggtagtgatga

SEQ ID NO: 89 - RNA, 1443 nt, Synthetic construct
  source 1..1443: mol_type = other RNA
atggagaccgacaccctcctgctgtgggtgctgctgctttgggtgcccgggagcacggggcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatga

SEQ ID NO: 90 - RNA, 1503 nt, Synthetic construct
  source 1..1503: mol_type = other RNA
atggagaccgacaccctcctgctgtgggtgctgctgctttgggtgcccgggagcacggggcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcaccaccacccccgccccccgcccccccacacccgcccccaccatcgcaagccagcccctgagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatga

SEQ ID NO: 91 - RNA, 1506 nt, Synthetic construct
  source 1..1506: mol_type = other RNA
atggctctcccagtgaccgccctgctgctgcccctagccctccttttgcacgcagcgaggcctcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcaccaccacccccgccccccgcccccccacacccgcccccaccatcgcaagccagcccctgagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatga

SEQ ID NO: 92 - RNA, 1482 nt, Synthetic construct
  source 1..1482: mol_type = other RNA
atggagaccgacaccctcctgctgtgggtgctgctgctttgggtgcccgggagcacggggcaagtcaccctgcgggagagcggccccgccctggtcaagaccacgcagaccctgaccctcacctgcaccttcagcggctttagcctcagcaccagcggcatgtgcgtgagctggatccgccagccccccgggaaggcactggagtggctcgccctgatcgactgggatgatgacaaatactacagcaccagcctgaagacccgcctgacgatctcaaaggataccagcaagagccaggtggtcctgaccatgaccagcatggaccccgtcgacaccgccacctactactgcgcccgcatcaggtacatctacggatacgactacttcgattactggggccagggaaccctggtgaccgtgtcgagcggggggggagggagcggcggcgggggcagcggcggcggcgggtccggcggggggggctccgacatccagatgacccagagccccagcagcctctccgccagcgtgggggatcgtgtgaccatcacctgtcgcgcgtcccaacgaataagcagccagctcaattggtaccagcagaagccagggaaagcacccaagctgctgatctacgcagccagcagcctgcagagcggcgtcccctcacgcttctcaggcagcggctccggaacagagttcacgctgacaatcagctccctgcagccagaggacatagcgacctattgctgccaacagacctacagcaagcccatcacgttcggccaaggaacccggctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 93 - RNA, 1494 nt, Synthetic construct
  source 1..1494: mol_type = other RNA
atggagaccgacaccctcctgctgtgggtgctgctgctttgggtgcccgggagcacgggggaggtgcagctggtggagagcggcggcggcctggtgcagcccggcaggagcctgaggctgagctgcgccgccagcggcttcaccttcgacggctacgccatgcactgggtgaggcaggcccccggcaagggcctggagtgggtgagcggcatcagctggaacagctacagcctgggctacgccgacagcgtgaagggcaggttcaccatcagcagggacaacgccaagaacagcctgtacctgcagatgaacagcctgaggatcgaggacaccgccctgtactactgcgccaaggagttcggctgggacctgccctacttcgactactggggccagggcaccctggtgaccgtgagcagcggcggcggcggcagcggcggcggcggcagcggcggcggcggcagcggcggcggcggcagcgacatcgtgatgacccagagccccgacagcctggccgtgagcctgggcgagagggccaccatcgactgcaagagcagccagaacgtgctgtacagcagcagcaacaagaactacctggcctggtaccagcagaagcccggccagccccccaacctgctgatctactgggccagcaccagggagagcggcgtgcccgacaggttcagcggcagcggcagcggcaccgacttcaccctgaccatcagcagcctgcaggccgaggacgtggccgtgtactactgccagcagtactacagcatcccctacaccttcggccagggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 94 - RNA, 1509 nt, Synthetic construct
  source 1..1509: mol_type = other RNA
atggagaccgacaccctcctgctgtgggtgctgctgctttgggtgcccgggagcacggggcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcgggcggcggggggagcggaggcggcggcagcggcggcggcggaagcgacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagcaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatga

SEQ ID NO: 95 - RNA, 1569 nt, Synthetic construct
  source 1..1569: mol_type = other RNA
atggagaccgacaccctcctgctgtgggtgctgctgctttgggtgcccgggagcacggggcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcgggcggcggggggagcggaggcggcggcagcggcggcggcggaagcgacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagccttagcaacagcatcatgtacttcagccactttgtccccgtcttcctgccagccaagccgaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatga

SEQ ID NO: 96 - RNA, 1464 nt, Synthetic construct
  source 1..1464: mol_type = other RNA
atggagaccgacaccctcctgctgtgggtgctgctgctttgggtgcccgggagcacggggcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcggacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagcaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatga

SEQ ID NO: 97 - protein, 487 aa, Synthetic construct
METDTLLLWVLLLWVPGSTGQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 487 aa, Synthetic construct
METDTLLLWVLLLWVPGSTGQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 494 aa, Synthetic construct
METDTLLLWVLLLWVPGSTGQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 478 aa, Synthetic construct
METDTLLLWVLLLWVPGSTGQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 498 aa, Synthetic construct
METDTLLLWVLLLWVPGSTGQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 499 aa, Synthetic construct
MALPVTALLLPLALLLHAARPQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 491 aa, Synthetic construct
METDTLLLWVLLLWVPGSTGQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 495 aa, Synthetic construct
METDTLLLWVLLLWVPGSTGEVQLVESGGGLVQPGRSLRLSCAASGFTFDGYAMHWVRQAPGKGLEWVSGISWNSYSLGYADSVKGRFTISRDNAKNSLYLQMNSLRIEDTALYYCAKEFGWDLPYFDYWGQGTLVTVSSGGGGSGGGGSGGGGSGGGGSDIVMTQSPDSLAVSLGERATIDCKSSQNVLYSSSNKNYLAWYQQKPGQPPNLLIYWASTRESGVPDRFSGSGSGTDFTLTISSLQAEDVAVYYCQQYYSIPYTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 500 aa, Synthetic construct
METDTLLLWVLLLWVPGSTGQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 520 aa, Synthetic construct
METDTLLLWVLLLWVPGSTGQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 485 aa, Synthetic construct
METDTLLLWVLLLWVPGSTGQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - RNA, 1473 nt, Synthetic construct
  source 1..1473: mol_type = other RNA
atggctctcccagtgaccgccctgctgctgcccctagccctccttttgcacgcagcgaggcctcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 109 - RNA, 1473 nt, Synthetic construct
  source 1..1473: mol_type = other RNA
atggctctcccagtgaccgccctgctgctgcccctagccctccttttgcacgcagcgaggcctcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 110 - RNA, 1446 nt, Synthetic construct
  source 1..1446: mol_type = other RNA
atggctctcccagtgaccgccctgctgctgcccctagccctccttttgcacgcagcgaggcctcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatga

SEQ ID NO: 111 - RNA, 1485 nt, Synthetic construct
  source 1..1485: mol_type = other RNA
atggctctcccagtgaccgccctgctgctgcccctagccctccttttgcacgcagcgaggcctcaagtcaccctgcgggagagcggccccgccctggtcaagaccacgcagaccctgaccctcacctgcaccttcagcggctttagcctcagcaccagcggcatgtgcgtgagctggatccgccagccccccgggaaggcactggagtggctcgccctgatcgactgggatgatgacaaatactacagcaccagcctgaagacccgcctgacgatctcaaaggataccagcaagagccaggtggtcctgaccatgaccagcatggaccccgtcgacaccgccacctactactgcgcccgcatcaggtacatctacggatacgactacttcgattactggggccagggaaccctggtgaccgtgtcgagcggggggggagggagcggcggcgggggcagcggcggcggcgggtccggcggggggggctccgacatccagatgacccagagccccagcagcctctccgccagcgtgggggatcgtgtgaccatcacctgtcgcgcgtcccaacgaataagcagccagctcaattggtaccagcagaagccagggaaagcacccaagctgctgatctacgcagccagcagcctgcagagcggcgtcccctcacgcttctcaggcagcggctccggaacagagttcacgctgacaatcagctccctgcagccagaggacatagcgacctattgctgccaacagacctacagcaagcccatcacgttcggccaaggaacccggctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 112 - RNA, 1497 nt, Synthetic construct
  source 1..1497: mol_type = other RNA
atggctctcccagtgaccgccctgctgctgcccctagccctccttttgcacgcagcgaggcctgaggtgcagctggtggagagcggcggcggcctggtgcagcccggcaggagcctgaggctgagctgcgccgccagcggcttcaccttcgacggctacgccatgcactgggtgaggcaggcccccggcaagggcctggagtgggtgagcggcatcagctggaacagctacagcctgggctacgccgacagcgtgaagggcaggttcaccatcagcagggacaacgccaagaacagcctgtacctgcagatgaacagcctgaggatcgaggacaccgccctgtactactgcgccaaggagttcggctgggacctgccctacttcgactactggggccagggcaccctggtgaccgtgagcagcggcggcggcggcagcggcggcggcggcagcggcggcggcggcagcggcggcggcggcagcgacatcgtgatgacccagagccccgacagcctggccgtgagcctgggcgagagggccaccatcgactgcaagagcagccagaacgtgctgtacagcagcagcaacaagaactacctggcctggtaccagcagaagcccggccagccccccaacctgctgatctactgggccagcaccagggagagcggcgtgcccgacaggttcagcggcagcggcagcggcaccgacttcaccctgaccatcagcagcctgcaggccgaggacgtggccgtgtactactgccagcagtactacagcatcccctacaccttcggccagggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 113 - RNA, 1467 nt, Synthetic construct
  source 1..1467: mol_type = other RNA
atggctctcccagtgaccgccctgctgctgcccctagccctccttttgcacgcagcgaggcctcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcggacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagcaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatga

SEQ ID NO: 114 - RNA, 1464 nt, Synthetic construct
  source 1..1464: mol_type = other RNA
atggctctcccagtgaccgccctgctgctgcccctagccctccttttgcacgcagcgaggcctgaggtgaagctgcaggagtccggccccgggctggtcgcgcccagccagagcctcagcgtgacctgcaccgtgagcggcgtgagcctgcccgactacggcgtcagctggatccgccagccccccaggaaggggctggagtggctgggcgtgatctggggcagtgagacgacctattacaactccgccctcaagagccgcctaaccatcatcaaggacaacagcaagagccaggtgttcctgaagatgaacagcctgcagaccgacgacaccgccatctactactgcgccaagcactactactacggcgggtcctacgccatggactactgggggcagggcaccagcgtgaccgtcagctcgggcggcggcggcagcgggggaggcgggtcggggggcggcggcagcgacatccagatgacccagaccaccagctccctgagcgcctccctcggcgaccgagtcacgatcagctgcagggcctcccaggacatcagcaagtacctcaactggtaccagcagaagccggacgggaccgtgaagctgctgatctaccacacctcccggctgcacagcggcgtgcccagccggttcagcggctccggcagcggcaccgactacagcctgaccatcagcaacctggagcaggaggacatcgccacctacttctgccagcaggggaacaccctgccctacacgttcggcggcggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatga

SEQ ID NO: 115 - RNA, 1473 nt, Synthetic construct
  source 1..1473: mol_type = other RNA
atggctctcccagtgaccgccctgctgctgcccctagccctccttttgcacgcagcgaggcctcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctggaagcgcggcaggaagaagctcctgtacatcttcaagcagccgttcatgcgcccggtgcagacgacccaggaggaggacgggtgcagctgccggttcccggaggaggaggagggcggctgcgagctgcgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatga

SEQ ID NO: 116 - protein, 488 aa, Synthetic construct [sequence omitted]

SEQ ID NO: 117 - protein, 488 aa, Synthetic construct
MALPVTALLLPLALLLHAARPQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 479 aa, Synthetic construct
MALPVTALLLPLALLLHAARPQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 492 aa, Synthetic construct
MALPVTALLLPLALLLHAARPQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 496 aa, Synthetic construct
MALPVTALLLPLALLLHAARPEVQLVESGGGLVQPGRSLRLSCAASGFTFDGYAMHWVRQAPGKGLEWVSGISWNSYSLGYADSVKGRFTISRDNAKNSLYLQMNSLRIEDTALYYCAKEFGWDLPYFDYWGQGTLVTVSSGGGGSGGGGSGGGGSGGGGSDIVMTQSPDSLAVSLGERATIDCKSSQNVLYSSSNKNYLAWYQQKPGQPPNLLIYWASTRESGVPDRFSGSGSGTDFTLTISSLQAEDVAVYYCQQYYSIPYTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 486 aa, Synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 485 aa, Synthetic construct
MALPVTALLLPLALLLHAARPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 488 aa, Synthetic construct
MALPVTALLLPLALLLHAARPQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKAAAIEVMYPPPYLDNEKSNGTIIHVKGKHLCPSPLFPGPSKPFWVLVVVGGVLACYSLLVTVAFIIFWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - RNA, 1473 nt, Synthetic construct
  source 1..1473: mol_type = other RNA
atggccctgcccgtgacagcactgctgctgccattagctctgctgctgcacgccgctagaccacaggtgcagctggtggagagcggaggaggagtggtccagcccggcagatccctcagactgagctgcgccgcttccggcttcaccttcagctcctacggcatgcactgggtgagacaggctccaggcaagggcctggaatgggtcgcaatcatctactacgatgggtctaagaagtattacgccgactccgtgaaggggaggttcacaatcagcagagacaatagcaagaatactctgtatctgcagatgaactccctgagagccgaggacacagctgtgtactactgcgcccgcgagctgactggcgatgcattcgacatctgggggcaggggaccaaggtgaccgtgtcctctggcggcggaggctctggaggaggaggctctggaggagggggcagcggcggaggaggaagtgagatcgtgctgacccagacccctagcagcctgagcgcctccctgggcgacagagtgaccatcagctgccgggcctctcagagcatcggcagctccctgcactggtaccagcagaagcctgaccagagcccaaagctgctgatcaagtacgccagccagagctttagcggcgtgcccagcaagttcatcggcagcggctccggcaccgatttcaccctgaccatcaattccctggaggccgaagatgctgccaccttctactgccaccagagcagcaccctgccctacaccttcggccagggcaccaagctggagatcaaagccgccgccatcgaagtgatgtacccccccccctacctggacaacgagaagtccaacggcaccatcatccacgtgaaaggcaagcacctgtgcccctcccccctgttccctggaccttccaagcctttctgggtgctggtggtggtgggcggcgtgctggcctgctacagcctgctggtgacagtggccttcatcatcttctgggtgagaagcaagcggtcccggctgctgcacagcgactacatgaacatgacccccagaagacccggccctaccagaaagcactaccagccctacgccccccccagagatttcgccgcctacagaagccgggtgaagttcagcagaagcgccgacgcccctgcctaccagcagggccagaatcagctgtacaacgagctgaacctgggcagaagagaggagtacgacgtgctggacaagagaagaggcagagatcccgagatgggcggcaagcccagacggaagaaccctcaggagggcctgtacaatgagctgcagaaggacaagatggccgaggcctacagcgagatcggcatgaagggcgagagaagaagaggcaagggccacgacggcctgtaccagggcctgagcaccgccacaaaggacacctacgatgccctgcacatgcaggccctgccccctagatagtgataa

SEQ ID NO: 125 - RNA, 1473 nt, Synthetic construct
  source 1..1473: mol_type = other RNA
atggccctgcccgtgacagcactgctgctgccattagctctgctgctgcacgccgctagaccacaggtgcagctggtggagagcggaggaggagtggtccagcccggcagatccctcagactgagctgcgccgcttccggcttcaccttcagctcctacggcatgcactgggtgagacaggctccaggcaagggcctggaatgggtcgcaatcatctactacgatgggtctaagaagtattacgccgactccgtgaaggggaggttcacaatcagcagagacaatagcaagaatactctgtatctgcagatgaactccctgagagccgaggacacagctgtgtactactgcgcccgcgagctgactggcgatgcattcgacatctgggggcaggggaccaaggtgaccgtgtcctctggcggcggaggctctggaggaggaggctctggaggagggggcagcggcggaggaggaagtgagatcgtgctgacccagacccctagcagcctgagcgcctccctgggcgacagagtgaccatcagctgccgggcctctcagagcatcggcagctccctgcactggtaccagcagaagcctgaccagagcccaaagctgctgatcaagtacgccagccagagctttagcggcgtgcccagcaagttcatcggcagcggctccggcaccgatttcaccctgaccatcaattccctggaggccgaagatgctgccaccttctactgccaccagagcagcaccctgccctacaccttcggccagggcaccaagctggagatcaaagccgccgccatcgaagtgatgtacccccccccctacctggacaacgagaagtccaacggcaccatcatccacgtgaaaggcaagcacctgtgcccctcccccctgttccctggaccttccaagcctttctgggtgctggtggtggtgggcggcgtgctggcctgctacagcctgctggtgacagtggccttcatcatcttctggaagcggggcagaaagaagctgctgtacatcttcaagcagcccttcatgcggcccgtgcagaccacccaggaggaggatggctgcagctgcagatttcctgaggaggaggagggcggctgcgagctgcgggtgaagttcagcagaagcgccgacgcccctgcctaccagcagggccagaatcagctgtacaacgagctgaacctgggcagaagagaggagtacgacgtgctggacaagagaagaggcagagatcccgagatgggcggcaagcccagacggaagaaccctcaggagggcctgtacaatgagctgcagaaggacaagatggccgaggcctacagcgagatcggcatgaagggcgagagaagaagaggcaagggccacgacggcctgtaccagggcctgagcaccgccacaaaggacacctacgatgccctgcacatgcaggccctgccccctagatagtgataa

SEQ ID NO: 126 - protein, 488 aa, Synthetic construct [sequence omitted]

SEQ ID NO: 127 - protein, 488 aa, Synthetic construct; identical to SEQ ID NO: 123

SEQ ID NO: 128 - RNA, 1626 nt, Synthetic construct
  source 1..1626: mol_type = other RNA
aggaaataagagagaaaagaagagtaagaagaaatataagagccaccatggagaccgacaccctcctgctgtgggtgctgctgctttgggtgcccgggagcacggggcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgagcggccgcttaattaagctgccttctgcggggcttgccttctggccatgcccttcttctctcccttgcacctgtacctcttggtctttgaataaagcctgagtaggaag

SEQ ID NO: 129 - RNA, 1626 nt, Synthetic construct
  source 1..1626: mol_type = other RNA
aggaaataagagagaaaagaagagtaagaagaaatataagagccaccatggagaccgacaccctcctgctgtgggtgctgctgctttgggtgcccgggagcacggggcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgagcggccgcttaattaagctgccttctgcggggcttgccttctggccatgcccttcttctctcccttgcacctgtacctcttggtctttgaataaagcctgagtaggaag

SEQ ID NO: 130 - RNA, 1647 nt, Synthetic construct
  source 1..1647: mol_type = other RNA
aggaaataagagagaaaagaagagtaagaagaaatataagagccaccatggagaccgacaccctcctgctgtgggtgctgctgctttgggtgcccgggagcacggggcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcgagcaccaccacccccgcgccccggccgccgacccccgcccccacaatcgcctcacagccactgagcctgcgccccgaggcctgcaggcccgccgcaggcggcgccgtgcacacgcgtgggctcgacttcgcctgtgacatatacatctgggcccccctggccggcacctgcggggtcctgctgctgagcctggtgatcaccctgtattgcaaccaccggaacgtgcgctccaaacgcagccggctgctgcacagcgactacatgaacatgaccccgcgcaggcccggcccgacccgaaagcactaccagccatacgcccccccccgggacttcgccgcctaccggtcaagggttaagttctcacggtcggccgacgcccccgcctaccagcagggccagaaccagctgtacaatgagctgaacctgggccggagggaggagtacgacgtactggataaaaggcgcggcagagaccccgagatgggggggaaacccaggcgtaagaacccccaggagggcctgtacaacgaactgcaaaaggacaagatggccgaggcctacagcgaaataggcatgaagggcgagcgtaggagggggaagggccacgacggcctgtatcaaggcctgtcgaccgccaccaaagacacctacgacgccctgcacatgcaggccctgccccccaggtagtgatgagcggccgcttaattaagctgccttctgcggggcttgccttctggccatgcccttcttctctcccttgcacctgtacctcttggtctttgaataaagcctgagtaggaag

SEQ ID NO: 131 - RNA, 1599 nt, Synthetic construct
  source 1..1599: mol_type = other RNA
aggaaataagagagaaaagaagagtaagaagaaatataagagccaccatggagaccgacaccctcctgctgtgggtgctgctgctttgggtgcccgggagcacggggcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatgagcggccgcttaattaagctgccttctgcggggcttgccttctggccatgcccttcttctctcccttgcacctgtacctcttggtctttgaataaagcctgagtaggaag

SEQ ID NO: 132 - RNA, 1659 nt, Synthetic construct
  source 1..1659: mol_type = other RNA
aggaaataagagagaaaagaagagtaagaagaaatataagagccaccatggagaccgacaccctcctgctgtgggtgctgctgctttgggtgcccgggagcacggggcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcaccaccacccccgccccccgcccccccacacccgcccccaccatcgcaagccagcccctgagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatgagcggccgcttaattaagctgccttctgcggggcttgccttctggccatgcccttcttctctcccttgcacctgtacctcttggtctttgaataaagcctgagtaggaag

SEQ ID NO: 133 - RNA, 1662 nt, Synthetic construct
  source 1..1662: mol_type = other RNA
aggaaataagagagaaaagaagagtaagaagaaatataagagccaccatggctctcccagtgaccgccctgctgctgcccctagccctccttttgcacgcagcgaggcctcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcaccaccacccccgccccccgcccccccacacccgcccccaccatcgcaagccagcccctgagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatgagcggccgcttaattaagctgccttctgcggggcttgccttctggccatgcccttcttctctcccttgcacctgtacctcttggtctttgaataaagcctgagtaggaag

SEQ ID NO: 134 - RNA, 1638 nt, Synthetic construct
  source 1..1638: mol_type = other RNA
aggaaataagagagaaaagaagagtaagaagaaatataagagccaccatggagaccgacaccctcctgctgtgggtgctgctgctttgggtgcccgggagcacggggcaagtcaccctgcgggagagcggccccgccctggtcaagaccacgcagaccctgaccctcacctgcaccttcagcggctttagcctcagcaccagcggcatgtgcgtgagctggatccgccagccccccgggaaggcactggagtggctcgccctgatcgactgggatgatgacaaatactacagcaccagcctgaagacccgcctgacgatctcaaaggataccagcaagagccaggtggtcctgaccatgaccagcatggaccccgtcgacaccgccacctactactgcgcccgcatcaggtacatctacggatacgactacttcgattactggggccagggaaccctggtgaccgtgtcgagcggggggggagggagcggcggcgggggcagcggcggcggcgggtccggcggggggggctccgacatccagatgacccagagccccagcagcctctccgccagcgtgggggatcgtgtgaccatcacctgtcgcgcgtcccaacgaataagcagccagctcaattggtaccagcagaagccagggaaagcacccaagctgctgatctacgcagccagcagcctgcagagcggcgtcccctcacgcttctcaggcagcggctccggaacagagttcacgctgacaatcagctccctgcagccagaggacatagcgacctattgctgccaacagacctacagcaagcccatcacgttcggccaaggaacccggctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgagcggccgcttaattaagctgccttctgcggggcttgccttctggccatgcccttcttctctcccttgcacctgtacctcttggtctttgaataaagcctgagtaggaag

SEQ ID NO: 135 - RNA, 1650 nt, Synthetic construct
  source 1..1650: mol_type = other RNA
aggaaataagagagaaaagaagagtaagaagaaatataagagccaccatggagaccgacaccctcctgctgtgggtgctgctgctttgggtgcccgggagcacgggggaggtgcagctggtggagagcggcggcggcctggtgcagcccggcaggagcctgaggctgagctgcgccgccagcggcttcaccttcgacggctacgccatgcactgggtgaggcaggcccccggcaagggcctggagtgggtgagcggcatcagctggaacagctacagcctgggctacgccgacagcgtgaagggcaggttcaccatcagcagggacaacgccaagaacagcctgtacctgcagatgaacagcctgaggatcgaggacaccgccctgtactactgcgccaaggagttcggctgggacctgccctacttcgactactggggccagggcaccctggtgaccgtgagcagcggcggcggcggcagcggcggcggcggcagcggcggcggcggcagcggcggcggcggcagcgacatcgtgatgacccagagccccgacagcctggccgtgagcctgggcgagagggccaccatcgactgcaagagcagccagaacgtgctgtacagcagcagcaacaagaactacctggcctggtaccagcagaagcccggccagccccccaacctgctgatctactgggccagcaccagggagagcggcgtgcccgacaggttcagcggcagcggcagcggcaccgacttcaccctgaccatcagcagcctgcaggccgaggacgtggccgtgtactactgccagcagtactacagcatcccctacaccttcggccagggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgagcggccgcttaattaagctgccttctgcggggcttgccttctggccatgcccttcttctctcccttgcacctgtacctcttggtctttgaataaagcctgagtaggaag

SEQ ID NO: 136 - RNA, 1650 nt, Synthetic construct; identical to SEQ ID NO: 135
  source 1..1650: mol_type = other RNA

SEQ ID NO: 137 - RNA, 1725 nt, Synthetic construct
  source 1..1725: mol_type = other RNA
aggaaataagagagaaaagaagagtaagaagaaatataagagccaccatggagaccgacaccctcctgctgtgggtgctgctgctttgggtgcccgggagcacggggcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcgggcggcggggggagcggaggcggcggcagcggcggcggcggaagcgacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagccttagcaacagcatcatgtacttcagccactttgtccccgtcttcctgccagccaagccgaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatgagcggccgcttaattaagctgccttctgcggggcttgccttctggccatgcccttcttctctcccttgcacctgtacctcttggtctttgaataaagcctgagtaggaag

SEQ ID NO: 138 - RNA, 1620 nt, Synthetic construct
  source 1..1620: mol_type = other RNA
aggaaataagagagaaaagaagagtaagaagaaatataagagccaccatggagaccgacaccctcctgctgtgggtgctgctgctttgggtgcccgggagcacggggcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcggacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagcaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatgagcggccgcttaattaagctgccttctgcggggcttgccttctggccatgcccttcttctctcccttgcacctgtacctcttggtctttgaataaagcctgagtaggaag

SEQ ID NO: 139 - RNA, 1580 nt, Synthetic construct
  source 1..1580: mol_type = other RNA
gggtcaagattagagaacggtcgtagcattatcggaggttctgccaccatggctctcccagtgaccgccctgctgctgcccctagccctccttttgcacgcagcgaggcctcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 140 - RNA, 1580 nt, Synthetic construct
  source 1..1580: mol_type = other RNA
gggtcaagattagagaacggtcgtagcattatcggaggttctgccaccatggctctcccagtgaccgccctgctgctgcccctagccctccttttgcacgcagcgaggcctcaggtgcagctggtgcagagcggcgccgaggtgaagaaacccggcgcgagcgtcaaagtgagctgcaaagcaagcggctatacattcaccggctactacatacactgggtgaggcaagccccaggccagggcctcgagtggatgggctggatcaatcccaactccggcggcacgaactatgcccagaagttccagggcagggtgaccatgacacgagacaccagcatcggcaccgcctacatggaactgtcaaggctgcgatccgacgacaccgccatttactactgcacccgggaggatacctggaactacatcgactattggggccaggggaccctcgtgacagtcagctccggggggggcgggagcggcggcgggggcagcggcggcggcggctcaggcggcggggggagcgacatccagatgacccaaagccccagcagcctctccgcgagcgtcggggaccgagtgaccatcacctgccgcgcctcccagagcatcagctcccatctcaactggtaccagcagaagcccggcaaggcccccaagctactgatctacgccgcctcaagcctccacagcggcgtacccagccgctttagcgggtccggcagcggcaccgacttcaccctcacgatctcctccctgcagcccgaggacttcgccacctactgctgccagcagagctacagcacccccaggacgttcggccagggcaccaaagtggaaatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 141 - RNA, 1553 nt, Synthetic construct
  source 1..1553: mol_type = other RNA
gggtcaagattagagaacggtcgtagcattatcggaggttctgccaccatggctctcccagtgaccgccctgctgctgcccctagccctccttttgcacgcagcgaggcctcaggtgaccctgagggagagcgggcccgccctggtgaagccgacacagacactgaccctcacgtgcaccttcagcggcatcagcctgacgacctccggcatgtgcgtgagctggatcaggcagccccccgggaaggccctggagtggctagccctcatcgactgggacggggacaactactactccacctcactcaagacaaggctgaccataagcaaggacaccagcaagaaccaggtcgtgctgaccatggccagcatggatccggtggataccgccacctactattgcgcccggatgggatacacctacggctacgacgcctttgacatctgggggcagggcacaatggtgacggtctcgtccggaggcggcggctccggagggggaggcagcggcggcggcggatcgggggggggcggctccgagatcgtcctcacccaaaccccctcaagcctgagcgcaagcctgggcgaccgcgtgaccatctcctgcagggccagccaatcaatcggcagcctgctccactggtaccagcagaaacccgaccagtccccaaagctgctgatcaagttcgccagccagtcactgagcggcgtgcccagccgcttcagcggcagcggctccggcaccgacttcaccctgacaataaactccctcgaagccgaggacgccgccacctactactgccaccagagcaggagcctgcccttcaccttcggacccggcaccaaggtggacatcaacgcaagcagcctgaggccggaggcatgccggccagcagccgggggcgcggtacacacccgcggcctggacttcgcgtgtgacatctacatctgggcccccctggccggcacgtgcggcgtcctgctgctgagcctcgtgatcacactgtactgcaaccaccggaacgtcaggtccaagaggagccgcctgctccacagcgactacatgaacatgaccccgaggcgacccggccccacgaggaaacactaccagccctacgccccacccagggacttcgcggcctatcgaagccgggtgaagttctcacgcagcgccgatgcccccgcctaccagcaggggcagaaccagctctacaacgaactgaacctgggacgacgggaggagtacgacgtactggataagcggcgtgggcgtgaccccgaaatgggcgggaagccccggaggaagaacccccaggagggactgtacaacgagctgcagaaggacaagatggcggaggcgtacagcgagatcggcatgaagggcgagcgaaggcgtggcaaggggcacgacggcctgtatcaggggctgtcaaccgcaaccaaggacacatacgacgccctccacatgcaagccctgcccccccgctagtgatgaggcacatttgaataaattctattaccagttcaagcttggcgtaatcatggtcatagctg

SEQ ID NO: 142 - RNA, 1592 nt, Synthetic construct
  source 1..1592: mol_type = other RNA
gggtcaagattagagaacggtcgtagcattatcggaggttctgccaccatggctctcccagtgaccgccctgctgctgcccctagccctccttttgcacgcagcgaggcctcaagtcaccctgcgggagagcggccccgccctggtcaagaccacgcagaccctgaccctcacctgcaccttcagcggctttagcctcagcaccagcggcatgtgcgtgagctggatccgccagccccccgggaaggcactggagtggctcgccctgatcgactgggatgatgacaaatactacagcaccagcctgaagacccgcctgacgatctcaaaggataccagcaagagccaggtggtcctgaccatgaccagcatggaccccgtcgacaccgccacctactactgcgcccgcatcaggtacatctacggatacgactacttcgattactggggccagggaaccctggtgaccgtgtcgagcggggggggagggagcggcggcgggggcagcggcggcggcgggtccggcggggggggctccgacatccagatgacccagagccccagcagcctctccgccagcgtgggggatcgtgtgaccatcacctgtcgcgcgtcccaacgaataagcagccagctcaattggtaccagcagaagccagggaaagcacccaagctgctgatctacgcagccagcagcctgcagagcggcgtcccctcacgcttctcaggcagcggctccggaacagagttcacgctgacaatcagctccctgcagccagaggacatagcgacctattgctgccaacagacctacagcaagcccatcacgttcggccaaggaacccggctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 143 - RNA, 1604 nt, Synthetic construct
  source 1..1604: mol_type = other RNA
gggtcaagattagagaacggtcgtagcattatcggaggttctgccaccatggctctcccagtgaccgccctgctgctgcccctagccctccttttgcacgcagcgaggcctgaggtgcagctggtggagagcggcggcggcctggtgcagcccggcaggagcctgaggctgagctgcgccgccagcggcttcaccttcgacggctacgccatgcactgggtgaggcaggcccccggcaagggcctggagtgggtgagcggcatcagctggaacagctacagcctgggctacgccgacagcgtgaagggcaggttcaccatcagcagggacaacgccaagaacagcctgtacctgcagatgaacagcctgaggatcgaggacaccgccctgtactactgcgccaaggagttcggctgggacctgccctacttcgactactggggccagggcaccctggtgaccgtgagcagcggcggcggcggcagcggcggcggcggcagcggcggcggcggcagcggcggcggcggcagcgacatcgtgatgacccagagccccgacagcctggccgtgagcctgggcgagagggccaccatcgactgcaagagcagccagaacgtgctgtacagcagcagcaacaagaactacctggcctggtaccagcagaagcccggccagccccccaacctgctgatctactgggccagcaccagggagagcggcgtgcccgacaggttcagcggcagcggcagcggcaccgacttcaccctgaccatcagcagcctgcaggccgaggacgtggccgtgtactactgccagcagtactacagcatcccctacaccttcggccagggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 144 - RNA, 1574 nt, Synthetic construct
  source 1..1574: mol_type = other RNA
gggtcaagattagagaacggtcgtagcattatcggaggttctgccaccatggctctcccagtgaccgccctgctgctgcccctagccctccttttgcacgcagcgaggcctcaggtgcagctgcagcagtccggcccagggctggtgaagccatcccagaccctgagcctgacctgcgccatctcaggcgacagcgtctccagcaactccgccgcctggaattggatccggcagagccccagcaggggcctcgagtggctggggcgtacctactaccgcagcaagtggtacaacgactacgcggtgagcgtgaagtcaaggatcaccatcaaccccgatacaagcaagaaccaattctcactgcagctgaactccgtgacgcccgaggacacagccgtgtactactgcgcccgggaggtgaccggcgacctggaagacgccttcgacatctggggacagggcacaatggtgacggtcagctccggcgggggggggtcggacatccagatgacgcagagccccagcagcctgagcgccagcgtaggagaccgcgtcaccatcacctgcagggcaagccagaccatctggagctacctgaactggtaccagcagcgtcccggcaaggcccccaatctgctgatctacgccgcctccagcctgcagtccggagtgccctcgaggttcagcggccggggcagcgggaccgacttcaccctgaccatcagctcgctgcaggccgaggactttgccacctactactgccaacagagctactcaatcccccagacgtttggccagggcaccaagctggagatcaaggccagcaccacaacccccgcgccccggccccccacccccgcccccaccatcgccagccagccgctgagcctgaggccggaagcgtgccgaccggccgccgggggcgccgtacacacccgcggactcgacttcgcctgcgacatatatatctgggcccccctggccggcacctgcggcgtcctgctcctgagcctggtgatcaccctgtactgcaaccacaggaacgtccggtccaagcgttcccgcctcctgcacagcgattacatgaacatgaccccccgacgccccggcccaacccgaaagcactaccagccatatgccccccccagggactttgccgcctatcgatccagggtgaagttcagcaggtcagcggacgcgcccgcctaccagcagggccagaaccagctgtataacgagctgaacctcgggcggcgagaggagtacgacgtgctcgacaagcgccggggcagggaccccgaaatgggagggaagccccgcaggaaaaacccccaggagggactctacaacgagctccaaaaggataagatggccgaagcctacagcgagatcggcatgaaaggggagaggaggcggggcaaggggcacgacggcctgtaccagggactgtcaaccgccaccaaggacacctacgacgccctgcacatgcaggccctgccgcccaggtagtgatgaggcacatttgaataaattctattaccagttcaagcttggcgtaatcatggtcatagctg

SEQ ID NO: 145 - RNA, 1571 nt, Synthetic construct
  source 1..1571: mol_type = other RNA
gggtcaagattagagaacggtcgtagcattatcggaggttctgccaccatggctctcccagtgaccgccctgctgctgcccctagccctccttttgcacgcagcgaggcctgaggtgaagctgcaggagtccggccccgggctggtcgcgcccagccagagcctcagcgtgacctgcaccgtgagcggcgtgagcctgcccgactacggcgtcagctggatccgccagccccccaggaaggggctggagtggctgggcgtgatctggggcagtgagacgacctattacaactccgccctcaagagccgcctaaccatcatcaaggacaacagcaagagccaggtgttcctgaagatgaacagcctgcagaccgacgacaccgccatctactactgcgccaagcactactactacggcgggtcctacgccatggactactgggggcagggcaccagcgtgaccgtcagctcgggcggcggcggcagcgggggaggcgggtcggggggcggcggcagcgacatccagatgacccagaccaccagctccctgagcgcctccctcggcgaccgagtcacgatcagctgcagggcctcccaggacatcagcaagtacctcaactggtaccagcagaagccggacgggaccgtgaagctgctgatctaccacacctcccggctgcacagcggcgtgcccagccggttcagcggctccggcagcggcaccgactacagcctgaccatcagcaacctggagcaggaggacatcgccacctacttctgccagcaggggaacaccctgccctacacgttcggcggcggcaccaagctggagatcaaggcggccgcgatagaggtaatgtaccctcctccttacctggacaacgagaagtcgaacggcaccatcatccacgtcaagggcaagcacctctgcccctcgcccctgttccccggcccctccaagcccttctgggtgctggtcgtggttggtggcgtcctcgcctgctactccctcctcgtcacggtggcgttcatcatcttctgggtgcggagcaagcgaagccgcctcctgcacagcgactacatgaacatgacgccccgccgccccggcccgacccggaagcactaccagccctacgccccgccgcgcgacttcgcggcctaccgctcccgggtcaagttctcccgcagcgccgacgcccccgcgtaccagcagggccagaaccagctctacaacgagctcaacctggggaggcgcgaggagtacgacgtgcttgacaagcgccgtggccgggaccctgagatggggggtaagccgcgacggaagaacccgcaggagggcctgtacaacgagctgcagaaggacaagatggcggaggcctacagcgagatcgggatgaagggggagcggcggcgtggcaaggggcacgacggcctgtaccagggcctcagtacggccaccaaggacacctacgacgccttgcacatgcaggccctccccccccgctagtgatgaggcacatttgaataaattctattaccagttcaagcttggcgtaatcatggtcatagctg

SEQ ID NO: 146 - RNA, 1580 nt, Synthetic construct
  source 1..1580: mol_type = other RNA
gggtcaagattagagaacggtcgtagcattatcggaggttctgccaccatggctctcccagtgaccgccctgctgctgcccctagccctccttttgcacgcagcgaggcctcaggtccagctcgtggagagcggcggcggcgtggtgcagcccggccgaagcctccgcctaagctgcgccgccagcgggttcaccttcagcagctacggcatgcactgggtacggcaggcccccggcaaaggactggagtgggtggcgatcatatactacgacgggagcaagaaatactacgccgactccgtcaagggcaggtttaccatcagccgcgacaacagcaagaacacgctgtacctgcagatgaacagcctgagggccgaggacaccgccgtctactactgtgcaagagagctaaccggcgacgcgtttgacatctgggggcagggcaccaaggtgaccgtgtcctccgggggcgggggcagcggcggcggcggctccggcggcggcgggtcaggcggcggcggcagcgagatagtgctgacccagaccccatccagcctgtccgcctccctgggcgaccgcgtgacgatcagctgccgggccagccagtcgatcggcagcagcctacactggtaccagcagaagccggaccagtcccccaagctgctcatcaagtacgcctcccagtcattctccggggtgccctcaaagttcatcgggagcgggtccggcacggacttcacactcaccatcaacagcctggaggccgaggacgccgcgaccttttactgccaccaaagcagcaccctcccctacaccttcggccagggcacaaagctggagatcaaggcggccgcgatagaggtaatgtaccctcctccttacctggacaacgagaagtcgaacggcaccatcatccacgtcaagggcaagcacctctgcccgtcgccgctgttcccgggcccgtccaagcccttctgggtgctggtcgtggttggtggcgtcctcgcctgctactccctcctcgtcacggtggcgttcatcatcttctggaagcgcggcaggaagaagctcctgtacatcttcaagcagccgttcatgcgcccggtgcagacgacccaggaggaggacgggtgcagctgccggttcccggaggaggaggagggcggctgcgagctgcgggtcaagttctcccgcagcgccgacgcgcccgcgtaccagcagggccagaaccagctctacaacgagctcaacctggggaggcgcgaggagtacgacgtgcttgacaagcgccgtggccgggaccctgagatgggcggtaagccgcgacggaagaacccgcaggagggcctgtacaacgagctgcagaaggacaagatggcggaggcctacagcgagatcgggatgaagggcgagcggcggcgtggcaagggccacgacggcctgtaccagggcctcagtacggccaccaaggacacctacgacgccttgcacatgcaggccctcccgccgcgctagtgatgaggcacatttgaataaattctattaccagttcaagcttggcgtaatcatggtcatagctg

SEQ ID NO: 147 - RNA, 1713 nt, Synthetic construct
  source 1..1713: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggccctgcccgtgacagcactgctgctgccattagctctgctgctgcacgccgctagaccacaggtgcagctggtggagagcggaggaggagtggtccagcccggcagatccctcagactgagctgcgccgcttccggcttcaccttcagctcctacggcatgcactgggtgagacaggctccaggcaagggcctggaatgggtcgcaatcatctactacgatgggtctaagaagtattacgccgactccgtgaaggggaggttcacaatcagcagagacaatagcaagaatactctgtatctgcagatgaactccctgagagccgaggacacagctgtgtactactgcgcccgcgagctgactggcgatgcattcgacatctgggggcaggggaccaaggtgaccgtgtcctctggcggcggaggctctggaggaggaggctctggaggagggggcagcggcggaggaggaagtgagatcgtgctgacccagacccctagcagcctgagcgcctccctgggcgacagagtgaccatcagctgccgggcctctcagagcatcggcagctccctgcactggtaccagcagaagcctgaccagagcccaaagctgctgatcaagtacgccagccagagctttagcggcgtgcccagcaagttcatcggcagcggctccggcaccgatttcaccctgaccatcaattccctggaggccgaagatgctgccaccttctactgccaccagagcagcaccctgccctacaccttcggccagggcaccaagctggagatcaaagccgccgccatcgaagtgatgtacccccccccctacctggacaacgagaagtccaacggcaccatcatccacgtgaaaggcaagcacctgtgcccctcccccctgttccctggaccttccaagcctttctgggtgctggtggtggtgggcggcgtgctggcctgctacagcctgctggtgacagtggccttcatcatcttctgggtgagaagcaagcggtcccggctgctgcacagcgactacatgaacatgacccccagaagacccggccctaccagaaagcactaccagccctacgccccccccagagatttcgccgcctacagaagccgggtgaagttcagcagaagcgccgacgcccctgcctaccagcagggccagaatcagctgtacaacgagctgaacctgggcagaagagaggagtacgacgtgctggacaagagaagaggcagagatcccgagatgggcggcaagcccagacggaagaaccctcaggagggcctgtacaatgagctgcagaaggacaagatggccgaggcctacagcgagatcggcatgaagggcgagagaagaagaggcaagggccacgacggcctgtaccagggcctgagcaccgccacaaaggacacctacgatgccctgcacatgcaggccctgccccctagatagtgataacgggtggcatccctgtgacccctccccagtgcctctcctggccctggaagttgccactccagtgcccaccagccttgtcctaataaaattaagttgcatc

SEQ ID NO: 148 - RNA, 1713 nt, Synthetic construct
  source 1..1713: mol_type = other RNA
ggacagatcgcctggagacgccatccacgctgttttgacctccatagaagacaccgggaccgatccagcctccgcggccgggaacggtgcattggaacgcggattccccgtgccaagagtgactcaccgtccttgacacgatggccctgcccgtgacagcactgctgctgccattagctctgctgctgcacgccgctagaccacaggtgcagctggtggagagcggaggaggagtggtccagcccggcagatccctcagactgagctgcgccgcttccggcttcaccttcagctcctacggcatgcactgggtgagacaggctccaggcaagggcctggaatgggtcgcaatcatctactacgatgggtctaagaagtattacgccgactccgtgaaggggaggttcacaatcagcagagacaatagcaagaatactctgtatctgcagatgaactccctgagagccgaggacacagctgtgtactactgcgcccgcgagctgactggcgatgcattcgacatctgggggcaggggaccaaggtgaccgtgtcctctggcggcggaggctctggaggaggaggctctggaggagggggcagcggcggaggaggaagtgagatcgtgctgacccagacccctagcagcctgagcgcctccctgggcgacagagtgaccatcagctgccgggcctctcagagcatcggcagctccctgcactggtaccagcagaagcctgaccagagcccaaagctgctgatcaagtacgccagccagagctttagcggcgtgcccagcaagttcatcggcagcggctccggcaccgatttcaccctgaccatcaattccctggaggccgaagatgctgccaccttctactgccaccagagcagcaccctgccctacaccttcggccagggcaccaagctggagatcaaagccgccgccatcgaagtgatgtacccccccccctacctggacaacgagaagtccaacggcaccatcatccacgtgaaaggcaagcacctgtgcccctcccccctgttccctggaccttccaagcctttctgggtgctggtggtggtgggcggcgtgctggcctgctacagcctgctggtgacagtggccttcatcatcttctggaagcggggcagaaagaagctgctgtacatcttcaagcagcccttcatgcggcccgtgcagaccacccaggaggaggatggctgcagctgcagatttcctgaggaggaggagggcggctgcgagctgcgggtgaagttcagcagaagcgccgacgcccctgcctaccagcagggccagaatcagctgtacaacgagctgaacctgggcagaagagaggagtacgacgtgctggacaagagaagaggcagagatcccgagatgggcggcaagcccagacggaagaaccctcaggagggcctgtacaatgagctgcagaaggacaagatggccgaggcctacagcgagatcggcatgaagggcgagagaagaagaggcaagggccacgacggcctgtaccagggcctgagcaccgccacaaaggacacctacgatgccctgcacatgcaggccctgccccctagatagtgataacgggtggcatccctgtgacccctccccagtgcctctcctggccctggaagttgccactccagtgcccaccagccttgtcctaataaaattaagttgcatc

SEQ ID NO: 149 - protein, 386 aa, Synthetic construct
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 150 - protein, 160 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFAC

SEQ ID NO: 151 - protein, 220 aa, Synthetic construct
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 6 aa, Synthetic construct
HHHHHH

SEQ ID NO: 153 - protein, 396 aa, Synthetic construct
LQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGWSHPQFEKGGGSGGGSGGSSAWSHPQFEK

SEQ ID NO: 154 - protein, 29 aa, Synthetic construct
WSHPQFEKGGGSGGGSGGSSAWSHPQFEK

SEQ ID NO: 155 - protein, 386 aa, Synthetic construct
NQFRVSPLGRTWNLGETVELKCQVLLSNPTSGCSWLFQPRGTAARPTFLLYLSQNKPKAAEGLDTQRFSGKRLGDTFVLTLRDFRQENEGYYFCSALSNSIMYFSHFVPVFLPAKPTTTPAPRPPTPAPTTASQPLSLRPEACRPAAGGSVNTRGLDFACPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGHHHHHH

SEQ ID NO: 156 - protein, 160 aa, Macaca fascicularis
NQFRVSPLGRTWNLGETVELKCQVLLSNPTSGCSWLFQPRGTAARPTFLLYLSQNKPKAAEGLDTQRFSGKRLGDTFVLTLRDFRQENEGYYFCSALSNSIMYFSHFVPVFLPAKPTTTPAPRPPTPAPTTASQPLSLRPEACRPAAGGSVNTRGLDFAC

SEQ ID NO: 157 - protein, 396 aa, Synthetic construct
LQQTPAYIQVQTNKMAMLSCEAKISLGNMRIYWLRQRQAPSSDSHHEFLALWDSTKGTVHGEEVAQEKIAVFRDASRFILNLTSVKPEDSGIYFCMIIGSPELTFGKGTQLSVVDFLPTTAQPTKKSTPKKRGCRLPRPATQKGPLCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGWSHPQFEKGGGSGGGSGGSSAWSHPQFEK

SEQ ID NO: 158 - protein, 147 aa, Macaca fascicularis
LQQTPAYIQVQTNKMAMLSCEAKISLGNMRIYWLRQRQAPSSDSHHEFLALWDSTKGTVHGEEVAQEKIAVFRDASRFILNLTSVKPEDSGIYFCMIIGSPELTFGKGTQLSVVDFLPTTAQPTKKSTPKKRGCRLPRPATQKGPLC

SEQ ID NO: 159 - protein, 1647 aa, Synthetic construct
AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCCGGGAGCACGGGGGACUACAAGGCGAAGGAGGUGCAGCUGCAGCAGAGCGGGGCCGAGCUGGUGAAGCCGGGCGCCAGCGUCAAGAUGAGCUGCAAGGCUAGCGGGUACACCUUCACCUCCUACAACAUGCACUGGGUUAAGCAGACCCCCGGGCAGGGCCUGGAGUGGAUCGGGGCCAUCUACCCAGGGAACGGGGACACCUCCUACAACCAGAAGUUCAAGGGCAAGGCCACGCUCACUGCGGACAAGUCCUCCAGCACAGCCUACAUGCAGCUGAGCAGCCUGACGAGCGAGGACUCGGCGGACUACUACUGCGCACGCAGCAACUACUACGGGUCCAGCUACUGGUUCUUCGACGUCUGGGGCGCGGGGACGACGGUCACCGUCAGCUCCGGCGGGGGCAGCGGCGGCGGGAGCGGGGGGGGCGGGAGCAGCGACAUCGUGCUGACCCAGUCCCCCGCCAUCCUGUCUGCGAGCCCCGGGGAGAAGGUCACCAUGACGUGCCGGGCCAGCUCAAGUGUGAACUACAUGGACUGGUACCAGAAGAAGCCGGGAUCCUCCCCCAAGCCCUGGAUUUACGCGACGUCCAACCUCGCGAGCGGGGUCCCUGCGCGCUUCUCUGGCUCCGGGUCGGGGACCAGCUACAGCCUCACCAUCAGCCGCGUGGAGGCCGAGGACGCGGCGACCUACUACUGCCAGCAGUGGAGCUUCAACCCCCCGACGUUCGGCGGGGGGACCAAGCUGGAGAUCAAGGGAGGGGGCGGAUCCGCGGCCGCGAUAGAGGUAAUGUACCCUCCUCCUUACCUGGACAACGAGAAGUCGAACGGCACCAUCAUCCACGUCAAGGGCAAGCACCUCUGCCCCUCGCCCCUGUUCCCCGGCCCCUCCAAGCCCUUCUGGGUGCUGGUCGUGGUUGGUGGCGUCCUCGCCUGCUACUCCCUCCUCGUCACGGUGGCGUUCAUCAUCUUCUGGGUGCGGAGCAAGCGAAGCCGCCUCCUGCACAGCGACUACAUGAACAUGACGCCCCGCCGCCCCGGCCCGACCCGGAAGCACUACCAGCCCUACGCCCCGCCGCGCGACUUCGCGGCCUACCGCUCCCGGGUCAAGUUCUCCCGCAGCGCCGAGCCCCCCGCGUACCAGCAGGGCCAGAACCAGCUCUACAACGAGCUGAACCUGGGGAGGCGCGAGGAGUACGACGUGCUUGACAAGCGCCGUGGCCGGGACCCUGAGAUGGGGGGUAAGCCGCGACGGAAGAACCCGCAGGAGGGCCUGUACAACGAGCUGCAGAAGGACAAGAUGGCGGAGGCCUACAGCGAGAUCGGGAUGAAGGGGGAGCGGCGGCGUGGCAAGGGGCACGACGGCCUGUACCAGGGCCUCAGUACGGCCACCAAGGACACCUACGACGCCUUGCACAUGCAGGCCCUCCCCCCCCGCUAGUGAUGAGCGGCCGCUUAAUUAAGCUGCCUUCUGCGGGGCUUGCCUUCUGGCCAUGCCCUUCUUCUCUCCCUUGCACCUGUACCUCUUGGUCUUUGAAUAAAGCCUGAGUAGGAAG

SEQ ID NO: 160 - protein, 132 aa, Synthetic construct
EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAKYSRPDPSENHVDMDYWGKGTLVTVSS

SEQ ID NO: 161 - protein, 132 aa, Synthetic construct; identical to SEQ ID NO: 1

SEQ ID NO: 162 - protein, 132 aa, Synthetic construct; identical to SEQ ID NO: 2

SEQ ID NO: 163 - protein, 132 aa, Synthetic construct; identical to SEQ ID NO: 3

SEQ ID NO: 164 - protein, 132 aa, Synthetic construct; identical to SEQ ID NO: 4

SEQ ID NO: 165 - protein, 132 aa, Synthetic construct; identical to SEQ ID NO: 5

SEQ ID NO: 166 - protein, 132 aa, Synthetic construct; identical to SEQ ID NO: 6

SEQ ID NO: 167 - protein, 132 aa, Synthetic construct; identical to SEQ ID NO: 7

SEQ ID NO: 168 - protein, 132 aa, Synthetic construct; identical to SEQ ID NO: 8

SEQ ID NO: 169 - protein, 132 aa, Synthetic construct; identical to SEQ ID NO: 9

SEQ ID NO: 170 - protein, 132 aa, Synthetic construct; identical to SEQ ID NO: 160

SEQ ID NO: 171 - protein, 132 aa, Synthetic construct; identical to SEQ ID NO: 1

SEQ ID NO: 172 - protein, 132 aa, Synthetic construct; identical to SEQ ID NO: 2

SEQ ID NO: 173 - protein, 132 aa, Synthetic construct; identical to SEQ ID NO: 3

SEQ ID NO: 174 - protein, 132 aa, Synthetic construct; identical to SEQ ID NO: 4

SEQ ID NO: 175 - protein, 132 aa, Synthetic construct; identical to SEQ ID NO: 5

SEQ ID NO: 176 - protein, 132 aa, Synthetic construct; identical to SEQ ID NO: 6

SEQ ID NO: 177 - protein, 132 aa, Synthetic construct; identical to SEQ ID NO: 7

SEQ ID NO: 178 - protein, 132 aa, Synthetic construct; identical to SEQ ID NO: 8

SEQ ID NO: 179 - protein, 132 aa, Synthetic construct; identical to SEQ ID NO: 9

SEQ ID NO: 180 - protein, 25 aa, Synthetic construct
EVQLVESGGGLVQAGGSLRLSCAAS

SEQ ID NO: 181 - protein, 10 aa, Synthetic construct
GFTFDDYAIG

SEQ ID NO: 182 - protein, 14 aa, Synthetic construct
WFRQAPGKEREEVS

SEQ ID NO: 183 - protein, 10 aa, Synthetic construct
CIRTYDGNTY

SEQ ID NO: 184 - protein, 39 aa, Synthetic construct
YIDSVKGRFTISSDNAKNTVYLQMNSLKPEDTAAYYCAA

SEQ ID NO: 185 - protein, 23 aa, Synthetic construct
GSYYACAKYSRPDPSENHVDMDY

SEQ ID NO: 186 - protein, 11 aa, Synthetic construct
WGKGTLVTVSS

SEQ ID NO: 187 - protein, 25 aa, Synthetic construct
EVQLVESGGGVVQPGGSLRLSCAAS

SEQ ID NO: 188 - protein, 10 aa, Synthetic construct
GFTFEDYAIG

SEQ ID NO: 189 - protein, 14 aa, Synthetic construct
WFRQAPGKEREEVS

SEQ ID NO: 190 - protein, 10 aa, Synthetic construct
CIRTYDEQTY

SEQ ID NO: 191 - protein, 39 aa, Synthetic construct
YADSVKGRFTISRDNAKNTVYLQMNSLRPEDTALYYCAA

SEQ ID NO: 192 - protein, 23 aa, Synthetic construct
GSYYACAKYSRPDPSEGHVDLDY

SEQ ID NO: 193 - protein, 11 aa, Synthetic construct
WGQGTLVTVSS

SEQ ID NO: 194 - protein, 25 aa, Synthetic construct
EVQLVESGGGVVQPGGSLRLSCAAS

SEQ ID NO: 195 - protein, 10 aa, Synthetic construct
GFTFEDYAIG

SEQ ID NO: 196 - protein, 14 aa, Synthetic construct
WFRQAPGKEREEVS

SEQ ID NO: 197 - protein, 10 aa, Synthetic construct
CIRTYDEQTY

SEQ ID NO: 198 - protein, 39 aa, Synthetic construct; identical to SEQ ID NO: 191

SEQ ID NO: 199 - protein, 23 aa, Synthetic construct
GSYYACAEYSRPDPSEGHVDLDY

SEQ ID NO: 200 - protein, 11 aa, Synthetic construct
WGQGTLVTVSS

SEQ ID NO: 201 - protein, 25 aa, Synthetic construct
EVQLVESGGGVVQPGGSLRLSCAAS

SEQ ID NO: 202 - protein, 10 aa, Synthetic construct
GFTFEDYAIG

SEQ ID NO: 203 - protein, 14 aa, Synthetic construct
WFRQAPGKEREEVS

SEQ ID NO: 204 - protein, 10 aa, Synthetic construct
CIRTYDEQTY

SEQ ID NO: 205 - protein, 39 aa, Synthetic construct; identical to SEQ ID NO: 191

SEQ ID NO: 206 - protein, 23 aa, Synthetic construct
GSYYACAYYSRPDPSEGHVDLDY

SEQ ID NO: 207 - protein, 11 aa, Synthetic construct
WGQGTLVTVSS

SEQ ID NO: 208 - protein, 25 aa, Synthetic construct
EVQLVESGGGVVQPGGSLRLSCAAS

SEQ ID NO: 209 - protein, 10 aa, Synthetic construct
GFTFEDYAIG

SEQ ID NO: 210 - protein, 14 aa, Synthetic construct
WFRQAPGKEREEVS

SEQ ID NO: 211 - protein, 10 aa, Synthetic construct
CIRTYDEQTY

SEQ ID NO: 212 - protein, 39 aa, Synthetic construct
YADSVKGRFTISRDNAKNTVDLQMNSLRPEDTALYYCAA

SEQ ID NO: 213 - protein, 23 aa, Synthetic construct
GSYYACAKYSRPDPSEGHVDLDY

SEQ ID NO: 214 - protein, 11 aa, Synthetic construct
WGQGTLVTVSS

SEQ ID NO: 215 - protein, 25 aa, Synthetic construct
EVQLVESGGGVVQPGGSLRLSCAAS

SEQ ID NO: 216 - protein, 10 aa, Synthetic construct
GFTFEDYAIG

SEQ ID NO: 217 - protein, 14 aa, Synthetic construct
WFRQAPGKEREEVS

SEQ ID NO: 218 - protein, 10 aa, Synthetic construct
CIRTYDEQTY

SEQ ID NO: 219 - protein, 39 aa, Synthetic construct; identical to SEQ ID NO: 212

SEQ ID NO: 220 - protein, 23 aa, Synthetic construct
GSYYACAEYSRPDPSEGHVDLDY

SEQ ID NO: 221 - protein, 11 aa, Synthetic construct
WGQGTLVTVSS

SEQ ID NO: 222 - protein, 25 aa, Synthetic construct
EVQLVESGGGVVQPGGSLRLSCAAS

SEQ ID NO: 223 - protein, 10 aa, Synthetic construct
GFTFEDYAIG

SEQ ID NO: 224 - protein, 14 aa, Synthetic construct
WFRQAPGKEREEVS

SEQ ID NO: 225 - protein, 10 aa, Synthetic construct
CIRTYDEQTY

SEQ ID NO: 226 - protein, 39 aa, Synthetic construct; identical to SEQ ID NO: 212

SEQ ID NO: 227 - protein, 23 aa, Synthetic construct
GSYYACAYYSRPDPSEGHVDLDY

SEQ ID NO: 228 - protein, 11 aa, Synthetic construct
WGQGTLVTVSS

SEQ ID NO: 229 - protein, 25 aa, Synthetic construct
EVQLVESGGGVVQPGGSLRLSCAAS

SEQ ID NO: 230 - protein, 10 aa, Synthetic construct
GFTFEDYAIG

SEQ ID NO: 231 - protein, 14 aa, Synthetic construct
WFRQAPGKEREEVS

SEQ ID NO: 232 - protein, 10 aa, Synthetic construct
CIRTYDEQTY

SEQ ID NO: 233 - protein, 39 aa, Synthetic construct
YADSVKGRFTISRDNAKNTVSLQMNSLRPEDTALYYCAA

SEQ ID NO: 234 - protein, 23 aa, Synthetic construct
GSYYACAKYSRPDPSEGHVDLDY

SEQ ID NO: 235 - protein, 11 aa, Synthetic construct
WGQGTLVTVSS

SEQ ID NO: 236 - protein, 25 aa, Synthetic construct
EVQLVESGGGVVQPGGSLRLSCAAS

SEQ ID NO: 237 - protein, 10 aa, Synthetic construct
GFTFEDYAIG

SEQ ID NO: 238 - protein, 14 aa, Synthetic construct
WFRQAPGKEREEVS

SEQ ID NO: 239 - protein, 10 aa, Synthetic construct
CIRTYDEQTY

SEQ ID NO: 240 - protein, 39 aa, Synthetic construct; identical to SEQ ID NO: 233

SEQ ID NO: 241 - protein, 23 aa, Synthetic construct
GSYYACAEYSRPDPSEGHVDLDY

SEQ ID NO: 242 - protein, 11 aa, Synthetic construct
WGQGTLVTVSS

SEQ ID NO: 243 - protein, 25 aa, Synthetic construct
EVQLVESGGGVVQPGGSLRLSCAAS

SEQ ID NO: 244 - protein, 10 aa, Synthetic construct
GFTFEDYAIG

SEQ ID NO: 245 - protein, 14 aa, Synthetic construct
WFRQAPGKEREEVS

SEQ ID NO: 246 - protein, 10 aa, Synthetic construct
CIRTYDEQTY

SEQ ID NO: 247 - protein, 39 aa, Synthetic construct; identical to SEQ ID NO: 233

SEQ ID NO: 248 - protein, 23 aa, Synthetic construct
GSYYACAYYSRPDPSEGHVDLDY

SEQ ID NO: 249 - protein, 11 aa, Synthetic construct
WGQGTLVTVSS

SEQ ID NO: 250 - protein, 30 aa, Synthetic construct
EVQLVESGGGLVQAGGSLRLSCAASGFTFD

SEQ ID NO: 251 - protein, 5 aa, Synthetic construct
DYAIG

SEQ ID NO: 252 - protein, 14 aa, Synthetic construct
WFRQAPGKEREEVS

SEQ ID NO: 253 - protein, 17 aa, Synthetic construct
CIRTYDGNTYYIDSVKG

SEQ ID NO: 254 - protein, 32 aa, Synthetic construct
RFTISSDNAKNTVYLQMNSLKPEDTAAYYCAA

SEQ ID NO: 255 - protein, 23 aa, Synthetic construct
GSYYACAKYSRPDPSENHVDMDY

SEQ ID NO: 256 - protein, 11 aa, Synthetic construct
WGKGTLVTVSS

SEQ ID NO: 257 - protein, 30 aa, Synthetic construct
EVQLVESGGGVVQPGGSLRLSCAASGFTFE

SEQ ID NO: 258 - protein, 5 aa, Synthetic construct
DYAIG

SEQ ID NO: 259 - protein, 14 aa, Synthetic construct
WFRQAPGKEREEVS

SEQ ID NO: 260 - protein, 17 aa, Synthetic construct
CIRTYDEQTYYADSVKG

SEQ ID NO: 261 - protein, 32 aa, Synthetic construct
RFTISRDNAKNTVYLQMNSLRPEDTALYYCAA

SEQ ID NO: 262 - protein, 23 aa, Synthetic construct
GSYYACAKYSRPDPSEGHVDLDY

SEQ ID NO: 263 - protein, 11 aa, Synthetic construct
WGQGTLVTVSS

SEQ ID NO: 264 - protein, 30 aa, Synthetic construct; identical to SEQ ID NO: 257

SEQ ID NO: 265 - protein, 5 aa, Synthetic construct
DYAIG

SEQ ID NO: 266 - protein, 14 aa, Synthetic construct
WFRQAPGKEREEVS

SEQ ID NO: 267 - protein, 17 aa, Synthetic construct
CIRTYDEQTYYADSVKG

SEQ ID NO: 268 - protein, 32 aa, Synthetic construct; identical to SEQ ID NO: 261

SEQ ID NO: 269 - protein, 23 aa, Synthetic construct
GSYYACAEYSRPDPSEGHVDLDY

SEQ ID NO: 270 - protein, 11 aa, Synthetic construct
WGQGTLVTVSS

SEQ ID NO: 271 - protein, 30 aa, Synthetic construct; identical to SEQ ID NO: 257

SEQ ID NO: 272 - protein, 5 aa, Synthetic construct
DYAIG

SEQ ID NO: 273 - protein, 14 aa, Synthetic construct
WFRQAPGKEREEVS

SEQ ID NO: 274 - protein, 17 aa, Synthetic construct
CIRTYDEQTYYADSVKG

SEQ ID NO: 275 - protein, 32 aa, Synthetic construct; identical to SEQ ID NO: 261

SEQ ID NO: 276 - protein, 23 aa, Synthetic construct
GSYYACAYYSRPDPSEGHVDLDY

SEQ ID NO: 277 - protein, 11 aa, Synthetic construct
WGQGTLVTVSS

SEQ ID NO: 278 - protein, 30 aa, Synthetic construct; identical to SEQ ID NO: 257

SEQ ID NO: 279 - protein, 5 aa, Synthetic construct
DYAIG

SEQ ID NO: 280 - protein, 14 aa, Synthetic construct
WFRQAPGKEREEVS

SEQ ID NO: 281 - protein, 17 aa, Synthetic construct
CIRTYDEQTYYADSVKG

SEQ ID NO: 282 - protein, 32 aa, Synthetic construct
RFTISRDNAKNTVDLQMNSLRPEDTALYYCAA

SEQ ID NO: 283 - protein, 23 aa, Synthetic construct
GSYYACAKYSRPDPSEGHVDLDY

SEQ ID NO: 284 - protein, 11 aa, Synthetic construct
WGQGTLVTVSS

SEQ ID NO: 285 - protein, 30 aa, Synthetic construct; identical to SEQ ID NO: 257

SEQ ID NO: 286 - protein, 5 aa, Synthetic construct
DYAIG

SEQ ID NO: 287 - protein, 14 aa, Synthetic construct
WFRQAPGKEREEVS

SEQ ID NO: 288 - protein, 17 aa, Synthetic construct
CIRTYDEQTYYADSVKG

SEQ ID NO: 289 - protein, 32 aa, Synthetic construct; identical to SEQ ID NO: 282

SEQ ID NO: 290 - protein, 23 aa, Synthetic construct
GSYYACAEYSRPDPSEGHVDLDY

SEQ ID NO: 291 - protein, 11 aa, Synthetic construct
WGQGTLVTVSS

SEQ ID NO: 292 - protein, 30 aa, Synthetic construct; identical to SEQ ID NO: 257

SEQ ID NO: 293 - protein, 5 aa, Synthetic construct
DYAIG

SEQ ID NO: 294 - protein, 14 aa, Synthetic construct
WFRQAPGKEREEVS

SEQ ID NO: 295 - protein, 17 aa, Synthetic construct
CIRTYDEQTYYADSVKG

SEQ ID NO: 296 - protein, 32 aa, Synthetic construct; identical to SEQ ID NO: 282

SEQ ID NO: 297 - protein, 23 aa, Synthetic construct
GSYYACAYYSRPDPSEGHVDLDY

SEQ ID NO: 298 - protein, 11 aa, Synthetic construct
WGQGTLVTVSS

SEQ ID NO: 299 - protein, 30 aa, Synthetic construct; identical to SEQ ID NO: 257

SEQ ID NO: 300 - protein, 5 aa, Synthetic construct
DYAIG

SEQ ID NO: 301 - protein, 14 aa, Synthetic construct
WFRQAPGKEREEVS

SEQ ID NO: 302 - protein, 17 aa, Synthetic construct
CIRTYDEQTYYADSVKG

SEQ ID NO: 303 - protein, 32 aa, Synthetic construct
RFTISRDNAKNTVSLQMNSLRPEDTALYYCAA

SEQ ID NO: 304 - protein, 23 aa, Synthetic construct
GSYYACAKYSRPDPSEGHVDLDY

SEQ ID NO: 305 - protein, 11 aa, Synthetic construct
WGQGTLVTVSS

SEQ ID NO: 306 - protein, 30 aa, Synthetic construct; identical to SEQ ID NO: 257

SEQ ID NO: 307 - protein, 5 aa, Synthetic construct
DYAIG

SEQ ID NO: 308 - protein, 14 aa, Synthetic construct
WFRQAPGKEREEVS

SEQ ID NO: 309 - protein, 17 aa, Synthetic construct
CIRTYDEQTYYADSVKG

SEQ ID NO: 310 - protein, 32 aa, Synthetic construct; identical to SEQ ID NO: 303

SEQ ID NO: 311 - protein, 23 aa, Synthetic construct
GSYYACAEYSRPDPSEGHVDLDY

SEQ ID NO: 312 - protein, 11 aa, Synthetic construct
WGQGTLVTVSS

SEQ ID NO: 313 - protein, 30 aa, Synthetic construct; identical to SEQ ID NO: 257

SEQ ID NO: 314 - protein, 5 aa, Synthetic construct
DYAIG

SEQ ID NO: 315 - protein, 14 aa, Synthetic construct
WFRQAPGKEREEVS

SEQ ID NO: 316 - protein, 17 aa, Synthetic construct
CIRTYDEQTYYADSVKG

SEQ ID NO: 317 - protein, 32 aa, Synthetic construct; identical to SEQ ID NO: 303

SEQ ID NO: 318 - protein, 23 aa, Synthetic construct
GSYYACAYYSRPDPSEGHVDLDY

SEQ ID NO: 319 - protein, 11 aa, Synthetic construct
WGQGTLVTVSS

SEQ ID NO: 320 - protein, 115 aa, Synthetic construct [sequence omitted]

SEQ ID NO: 321 - protein, 115 aa, Synthetic construct [sequence omitted]

SEQ ID NO: 322 - protein, 116 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 323 - protein, 116 aa, Synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 324 - protein, 115 aa, Synthetic construct [sequence omitted]

SEQ ID NO: 325 - protein, 116 aa, Synthetic construct
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 326 - protein, 115 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 327 - protein, 116 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 328 - protein, 117 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAA

SEQ ID NO: 329 - protein, 118 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSAAA

SEQ ID NO: 330 - protein, 116 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSG

SEQ ID NO: 331 - protein, 117 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGG

SEQ ID NO: 332 - protein, 118 aa, Synthetic construct
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSGGG

SEQ ID NO: 333 - protein, 116 aa, Synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 334 - protein, 123 aa, Synthetic construct
EVQLVESGGGVVQPGGSLRLSCAASGLTFSSYAMGWFRQAPGKERERVVSISRGGGYTYYADSVKGRFTISRDNSENTVYLQMNSLRPEDTALYYCAAARYWATGSEYEFDYWGQGTLVTVSS

SEQ ID NO: 335 - protein, 115 aa, Synthetic construct
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 336 - skipped

SEQ ID NO: 337 - protein, 5 aa, Synthetic construct
GGGGS

SEQ ID NO: 338 - protein, 7 aa, Synthetic construct
SGGSGGS

SEQ ID NO: 339 - protein, 8 aa, Synthetic construct
GGGGSGGS

SEQ ID NO: 340 - protein, 9 aa, Synthetic construct
GGGGSGGGS

SEQ ID NO: 341 - protein, 10 aa, Synthetic construct
GGGGSGGGGS

SEQ ID NO: 342 - protein, 15 aa, Synthetic construct
GGGGSGGGGSGGGGS

SEQ ID NO: 343 - protein, 18 aa, Synthetic construct
GGGGSGGGGSGGGGSGGS

SEQ ID NO: 344 - protein, 20 aa, Synthetic construct
GGGGSGGGGSGGGGSGGGGS

SEQ ID NO: 345 - protein, 25 aa, Synthetic construct
GGGGSGGGGSGGGGSGGGGSGGGGS

SEQ ID NO: 346 - protein, 30 aa, Synthetic construct
GGGGSGGGGSGGGGSGGGGSGGGGSGGGGS

SEQ ID NO: 347 - protein, 35 aa, Synthetic construct
GGGGSGGGGSGGGGSGGGGSGGGGSGGGGSGGGGS

SEQ ID NO: 348 - protein, 40 aa, Synthetic construct
GGGGSGGGGSGGGGSGGGGSGGGGSGGGGSGGGGSGGGGS

SEQ ID NO: 349 - protein, 15 aa, Synthetic construct
EPKSCDKTHTCPPCP

SEQ ID NO: 350 - protein, 24 aa, Synthetic construct
GGGGSGGGSEPKSCDKTHTCPPCP

SEQ ID NO: 351 - protein, 12 aa, Synthetic construct
EPKTPKPQPAAA

SEQ ID NO: 352 - protein, 62 aa, Synthetic construct
ELKTPLGDTTHTCPRCPEPKSCDTPPPCPRCPEPKSCDTPPPCPRCPEPKSCDTPPPCPRCP

SEQ ID NO: 353 - protein, 6 aa, Synthetic construct
  VARIANT 6: note = Can be present in repeats of 1-10, preferably 1-5, such as 1, 2, 3, 4, or 5; each is any amino acid that is independently chosen
VTVSSX

SEQ ID NO: 354 - protein, 5 aa, Synthetic construct
VKVSS

SEQ ID NO: 355 - protein, 5 aa, Synthetic construct
VQVSS

SEQ ID NO: 356 - protein, 5 aa, Synthetic construct
VTVKS

SEQ ID NO: 357 - protein, 5 aa, Synthetic construct
VTVQS

SEQ ID NO: 358 - protein, 5 aa, Synthetic construct
VKVKS

SEQ ID NO: 359 - protein, 5 aa, Synthetic construct
VKVQS

SEQ ID NO: 360 - protein, 5 aa, Synthetic construct
VQVKS

SEQ ID NO: 361 - protein, 5 aa, Synthetic construct
VQVQS

SEQ ID NO: 362 - protein, 6 aa, Synthetic construct
VTVSSA

SEQ ID NO: 363 - protein, 6 aa, Synthetic construct
VKVSSA

SEQ ID NO: 364 - protein, 6 aa, Synthetic construct
VQVSSA

SEQ ID NO: 365 - protein, 6 aa, Synthetic construct
VTVKSA

SEQ ID NO: 366 - protein, 6 aa, Synthetic construct
VTVQSA

SEQ ID NO: 367 - protein, 6 aa, Synthetic construct
VKVKSA

SEQ ID NO: 368 - protein, 6 aa, Synthetic construct
VKVQSA

SEQ ID NO: 369 - protein, 6 aa, Synthetic construct
VQVKSA

SEQ ID NO: 370 - protein, 6 aa, Synthetic construct
VQVQSA

SEQ ID NO: 371 - protein, 5 aa, Synthetic construct
VTVSS

SEQ ID NO: 372 - protein, 4 aa, Synthetic construct
KERE

SEQ ID NO: 373 - protein, 4 aa, Synthetic construct
KQRE

SEQ ID NO: 374 - protein, 4 aa, Synthetic construct
GLEW

SEQ ID NO: 375 - protein, 5 aa, Synthetic construct
KEREL

SEQ ID NO: 376 - protein, 5 aa, Synthetic construct
KEREF

SEQ ID NO: 377 - protein, 5 aa, Synthetic construct
KQREL

SEQ ID NO: 378 - protein, 5 aa, Synthetic construct
KQREF

SEQ ID NO: 379 - protein, 5 aa, Synthetic construct
KEREG

SEQ ID NO: 380 - protein, 5 aa, Synthetic construct
KQREW

SEQ ID NO: 381 - protein, 5 aa, Synthetic construct
KQREG

SEQ ID NO: 382 - protein, 4 aa, Synthetic construct
TERE

SEQ ID NO: 383 - protein, 5 aa, Synthetic construct
TEREL

SEQ ID NO: 384 - protein, 4 aa, Synthetic construct
TQRE

SEQ ID NO: 385 - protein, 5 aa, Synthetic construct
TQREL

SEQ ID NO: 386 - protein, 4 aa, Synthetic construct
KECE

SEQ ID NO: 387 - protein, 5 aa, Synthetic construct
KECEL

SEQ ID NO: 388 - protein, 5 aa, Synthetic construct
KECER

SEQ ID NO: 389 - protein, 4 aa, Synthetic construct
KQCE

SEQ ID NO: 390 - protein, 5 aa, Synthetic construct
KQCEL

SEQ ID NO: 391 - protein, 4 aa, Synthetic construct
RERE

SEQ ID NO: 392 - protein, 5 aa, Synthetic construct
REREG

SEQ ID NO: 393 - protein, 4 aa, Synthetic construct
RQRE

SEQ ID NO: 394 - protein, 5 aa, Synthetic construct
RQREL

SEQ ID NO: 395 - protein, 5 aa, Synthetic construct
RQREF

SEQ ID NO: 396 - protein, 5 aa, Synthetic construct
RQREW

SEQ ID NO: 397 - protein, 4 aa, Synthetic construct
QERE

SEQ ID NO: 398 - protein, 5 aa, Synthetic construct
QEREG

SEQ ID NO: 399 - protein, 4 aa, Synthetic construct
QQRE

SEQ ID NO: 400 - protein, 5 aa, Synthetic construct
QQREW

SEQ ID NO: 401 - protein, 5 aa, Synthetic construct
QQREL

SEQ ID NO: 402 - protein, 5 aa, Synthetic construct
QQREF

SEQ ID NO: 403 - protein, 4 aa, Synthetic construct
KGRE

SEQ ID NO: 404 - protein, 5 aa, Synthetic construct
KGREG

SEQ ID NO: 405 - protein, 4 aa, Synthetic construct
KDRE

SEQ ID NO: 406 - protein, 5 aa, Synthetic construct
KDREV

SEQ ID NO: 407 - protein, 5 aa, Synthetic construct
DECKL

SEQ ID NO: 408 - protein, 5 aa, Synthetic construct
NVCEL

SEQ ID NO: 409 - protein, 4 aa, Synthetic construct
GVEW

SEQ ID NO: 410 - protein, 4 aa, Synthetic construct
EPEW

SEQ ID NO: 411 - protein, 4 aa, Synthetic construct
GLER

SEQ ID NO: 412 - protein, 4 aa, Synthetic construct
DQEW

SEQ ID NO: 413 - protein, 4 aa, Synthetic construct
DLEW

SEQ ID NO: 414 - protein, 4 aa, Synthetic construct
GIEW

SEQ ID NO: 415 - protein, 4 aa, Synthetic construct
ELEW

SEQ ID NO: 416 - protein, 4 aa, Synthetic construct
GPEW

SEQ ID NO: 417 - protein, 4 aa, Synthetic construct
EWLP

SEQ ID NO: 418 - protein, 4 aa, Synthetic construct
GPER

SEQ ID NO: 419 - protein, 132 aa, Synthetic construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 420 - protein, 25 aa, Synthetic construct
EVQLVESGGGLVQAGGSLRLSCAAS

SEQ ID NO: 421 - protein, 8 aa, Synthetic construct
GSTFSDYG

SEQ ID NO: 422 - protein, 17 aa, Synthetic construct
VGWFRQAPGKGREFVAD

SEQ ID NO: 423 - protein, 8 aa, Synthetic construct
IDWNGEHT

SEQ ID NO: 424 - protein, 38 aa, Synthetic construct
SYADSVKGRFATSRDNAKNTAYLQMNSLKPEDTAVYYC

SEQ ID NO: 425 - protein, 14 aa, Synthetic construct
AADALPYTVRKYNY

SEQ ID NO: 426 - protein, 22 aa, Synthetic construct
WGQGTQVTVSSGGCGGHHHHHH

SEQ ID NO: 427 - protein, 5 aa, Synthetic construct
SYGMH

SEQ ID NO: 428 - protein, 17 aa, Synthetic construct
IIYYDGSKKYYADSVKG

SEQ ID NO: 429 - protein, 9 aa, Synthetic construct
ELTGDAFDI

SEQ ID NO: 430 - protein, 11 aa, Synthetic construct
RASQSIGSSLH

SEQ ID NO: 431 - protein, 7 aa, Synthetic construct
YASQSFS

SEQ ID NO: 432 - protein, 9 aa, Synthetic construct
HQSSTLPYT

SEQ ID NO: 433 - protein, 118 aa, Synthetic construct
QVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSS

SEQ ID NO: 434 - protein, 107 aa, Synthetic construct
EIVLTQSPDFQSVTPKEKVTITCRASQSIGSSLHWYQQKPDQSPKLLIKYASQSFSGVPSKFIGSGSGTDFTLTINSLEAEDAATFYCHQSSTLPYTFGQGTKLEIK

SEQ ID NO: 435 - protein, 329 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 436 - protein, 107 aa, Synthetic construct [sequence omitted]

SEQ ID NO: 437 - protein, 447 aa, Synthetic construct
QVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 438 - protein, 214 aa, Synthetic construct
EIVLTQSPDFQSVTPKEKVTITCRASQSIGSSLHWYQQKPDQSPKLLIKYASQSFSGVPSKFIGSGSGTDFTLTINSLEAEDAATFYCHQSSTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 439 - protein, 354 aa, Synthetic construct
CAGGTGCAGCTGGTGGAGTCGGGGGGAGGCGTGGTCCAGCCTGGGAGGTCCCTGAGACTCTCCTGTGCAGCGTCTGGATTCACCTTCAGTAGCTATGGCATGCACTGGGTCCGCCAGGCTCCAGGCAAGGGGCTGGAGTGGGTGGCAATTATATACTATGATGGAAGTAAAAAATACTATGCAGACTCCGTGAAGGGCCGATTCACCATCTCCAGAGACAATTCCAAGAACACGTTGTATCTGCAAATGAACAGCCTGAGAGCCGAGGACACGGCTGTGTATTACTGTGCGAGAGAGCTGACTGGGGATGCTTTTGATATCTGGGGCCAAGGGACAAAGGTCACCGTCTCTTCA

SEQ ID NO: 440 - protein, 321 aa, Synthetic construct
GAAATTGTGCTGACTCAGTCTCCAGACTTTCAGTCTGTGACTCCAAAGGAGAAAGTCACCATCACCTGCCGGGCCAGTCAGAGCATTGGTAGTAGCTTACACTGGTACCAGCAGAAACCAGATCAGTCTCCAAAGCTCCTCATCAAGTATGCTTCCCAGTCCTTCTCAGGGGTCCCCTCGAAGTTCATTGGCAGTGGATCTGGGACAGATTTCACCCTCACCATCAATAGCCTGGAAGCTGAAGATGCTGCAACGTTTTACTGTCATCAGAGTAGTACTTTACCGTACACTTTTGGCCAGGGGACCAAGCTGGAGATCAAA

SEQ ID NO: 441 - protein, 1341 aa, Synthetic construct
CAGGTGCAGCTGGTGGAGTCGGGGGGAGGCGTGGTCCAGCCTGGGAGGTCCCTGAGACTCTCCTGTGCAGCGTCTGGATTCACCTTCAGTAGCTATGGCATGCACTGGGTCCGCCAGGCTCCAGGCAAGGGGCTGGAGTGGGTGGCAATTATATACTATGATGGAAGTAAAAAATACTATGCAGACTCCGTGAAGGGCCGATTCACCATCTCCAGAGACAATTCCAAGAACACGTTGTATCTGCAAATGAACAGCCTGAGAGCCGAGGACACGGCTGTGTATTACTGTGCGAGAGAGCTGACTGGGGATGCTTTTGATATCTGGGGCCAAGGGACAAAGGTCACCGTCTCTTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 442 - protein, 642 aa, Synthetic construct
GAAATTGTGCTGACTCAGTCTCCAGACTTTCAGTCTGTGACTCCAAAGGAGAAAGTCACCATCACCTGCCGGGCCAGTCAGAGCATTGGTAGTAGCTTACACTGGTACCAGCAGAAACCAGATCAGTCTCCAAAGCTCCTCATCAAGTATGCTTCCCAGTCCTTCTCAGGGGTCCCCTCGAAGTTCATTGGCAGTGGATCTGGGACAGATTTCACCCTCACCATCAATAGCCTGGAAGCTGAAGATGCTGCAACGTTTTACTGTCATCAGAGTAGTACTTTACCGTACACTTTTGGCCAGGGGACCAAGCTGGAGATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 443 - protein, 5 aa, Synthetic construct
NYGMH

SEQ ID NO: 444 - protein, 17 aa, Synthetic construct
VIYYDGSKNYYEDSVKG

SEQ ID NO: 445 - protein, 11 aa, Synthetic construct
RASQRIGSSLH

SEQ ID NO: 446 - protein, 9 aa, Synthetic construct
HQSSSFPYT

SEQ ID NO: 447 - protein, 118 aa, Synthetic construct
QVQLVESGGDVVQPGRSLRLSCAASGFIFRNYGMHWVRQAPGKGLEWVAVIYYDGSKNYYEDSVKGRFTISRDNSKNTLYLQMNSLRVEDTAVYYCARELTGDAFDIWGQGTMVTVSS

SEQ ID NO: 448 - protein, 107 aa, Synthetic construct
EIVLTQSPDFQSLTPKEKVTITCRASQRIGSSLHWYQQKPDQSPKLLIKYASQSFSGVPSRFSGSGSGTDFTLTINSLDAEDAATYYCHQSSSFPYTFGQGTKLQIK

SEQ ID NO: 449 - protein, 17 aa, Synthetic construct
VIYYDGNKKYYVDSVKD

SEQ ID NO: 450 - protein, 9 aa, Synthetic construct
HQSSTFPYT

SEQ ID NO: 451 - protein, 118 aa, Synthetic construct
QVQLVESGGGVVQPGRSLRLSCAASGFTFSSYGMHWVRQAPGKGLEWVAVIYYDGNKKYYVDSVKDRITISRDNSKNTLYLQMNSLRAEDTAVYYCARELTGDAFDIWGQGTMVTVSS

SEQ ID NO: 452 - protein, 107 aa, Synthetic construct
EIVLTQSPDFQSVTPKEKVTITCRASQRIGSSLHWYQQKSDQSPKLLIKYASQSFSGVPSRFSGSGSGTDFTLTINSLEAEDAATYYCHQSSTFPYTFGQGTKLEIK

SEQ ID NO: 453 - protein, 17 aa, Synthetic construct
VIYYDGNNKYYADSVKG

SEQ ID NO: 454 - protein, 9 aa, Synthetic construct
ELTGDAFDL

SEQ ID NO: 455 - protein, 11 aa, Synthetic construct
RASQSIGSHLH

SEQ ID NO: 456 - protein, 9 aa, Synthetic construct
HQSSRFPYT

SEQ ID NO: 457 - protein, 118 aa, Synthetic construct
QVQLVESGGGVVQPGRSLRLSCAASGFTFSSYGMHWVRQTPGKGLEWVAVIYYDGNNKYYADSVKGRFTISRENSKNTLYLQMNSLRADDTALYYCARELTGDAFDLWGQGTMVTVSS

SEQ ID NO: 458 - protein, 107 aa, Synthetic construct
EIVLTQSPDFQSATPKEKVTITCRASQSIGSHLHWYQQKPDQSPKLLIKYASQSFSGVPSRFSGSGSGTDFTLTINRLEDEDAATYYCHQSSRFPYTFGQGTKLDIK

SEQ ID NO: 459 - protein, 8 aa, Synthetic construct
  VARIANT 3: note = T or I
  VARIANT 5: note = S or R
  VARIANT 6: note = S or N
GFXFXXYG

SEQ ID NO: 460 - protein, 8 aa, Synthetic construct
  VARIANT 6: note = N or S
  VARIANT 7: note = K or N
  VARIANT 8: note = K or N
IYYDGXXX

SEQ ID NO: 461 - protein, 11 aa, Synthetic construct
  VARIANT 11: note = I or L
ARELTGDAFDX

SEQ ID NO: 462 - protein, 6 aa, Synthetic construct
  VARIANT 2: note = S or R
  VARIANT 6: note = S or H
QXIGSX

SEQ ID NO: 463 - protein, 643 aa, Mus musculus
SANDWTVDHPQTLFAWEGACIRIPCKYKTPLPKARLDNILLFQNYEFDKATKKFTGTVLYNATKTEKDPESELYLSKQGRVTFLGNRIDNCTLKIHPIRANDSGNLGLRMTAGTERWMEPIHLNVSEKPFQPYIQMPSEIRESQSVTLTCGLNFSCFGYDILLKWFLEDSEITSITSSVTSITSSVTSSIKNVYTESKLTFQPKWTDHGKSVKCQVQHSSKVLSERTVRLDVKYTPKLEIKVNPTEVEKNNSVTMTCRVNSSNPKLRTVAVSWFKDGRPLEDQELEQEQQMSKLILHSVTKDMRGKYRCQASNDIGPGESEEVELTVHYAPEPSRVHIYPSPAEEGQSVELICESLASPSATNYTWYHNRKPIPGDTQEKLRIPKVSPWHAGNYSCLAENRLGHGKIDQEAKLDVHYAPKAVTTVIQSFTPILEGDSVTLVCRYNSSNPDVTSYRWNPQGSGSVLKPGVLRIQKVTWDSMPVSCAACNHKCSWALPVILNVHYAPRDVKVLKVSPASEIRAGQRVLLQCDFAESNPAEVRFFWKKNGSLVQEGRYLSFGSVSPEDSGNYNCMVNNSIGETLSQAWNLQVLYAPRRLRVSISPGDHVMEGKKATLSCESDANPPISQYTWFDSSGQDLHSSGQK

SEQ ID NO: 464 - protein, 9 aa, Synthetic construct
  VARIANT 5: note = T, R, or S
HQSSXEPYT

SEQ ID NO: 465 - DNA, 354 bp, Synthetic construct
  source 1..354: mol_type = other DNA
caggtgcagctggtggaatctgggggagacgtggtccagcctgggaggtccctgagactctcctgtgcagcgtctggattcatcttcaggaactatggcatgcactgggtccgccaggctccaggcaaggggctggagtgggtggcagtcatttactatgatggaagtaaaaattactatgaagactccgtgaagggccgattcaccatctccagagacaattccaaaaacacgctgtatctgcaaatgaacagcctgagagtcgaggacacggctgtgtattactgtgcgagagaacttactggggatgcttttgatatctggggccaagggacaatggtcaccgtctcttca

SEQ ID NO: 466 - DNA, 321 bp, Synthetic construct
  source 1..321: mol_type = other DNA
gaaattgtgctgactcagtctccagactttcagtctctgactccaaaggagaaagtcaccatcacctgtcgggccagtcagagaattggtagtagcttacactggtaccagcagaaaccagatcagtctccaaaactcctcatcaagtatgcttcccagtccttctcaggggtcccctcgaggttcagtggcagtggatctgggacagatttcaccctcaccatcaatagcctggatgctgaagatgctgcaacgtattactgtcatcagagtagtagtttcccgtacacttttggccaggggaccaagctgcagatcaaa

SEQ ID NO: 467 - DNA, 354 bp, Synthetic construct
  source 1..354: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcggttatatactatgatggaaataaaaaatactatgttgactccgtgaaggaccgaatcaccatctccagagacaattccaagaacacgttgtatctgcaaatgaacagcctgagagccgaggacacggctgtgtattattgtgcgagagaactaactggggatgcttttgatatctggggccaagggacaatggtcaccgtctcttca

SEQ ID NO: 468 - DNA, 321 bp, Synthetic construct
  source 1..321: mol_type = other DNA
gaaattgtgctgactcagtctccagactttcagtctgtgactccaaaggagaaagtcaccatcacctgccgggccagtcagagaattggaagtagcttacactggtaccagcagaaatcagatcagtctccaaagctcctcatcaagtatgcttcccagtccttctcaggggtcccctcgaggttcagtggcagtggatctgggacagatttcaccctcaccatcaatagcctggaagctgaagatgctgcaacgtattactgtcatcagagtagtacgttcccgtacacttttggccaggggaccaagctagagatcaaa

SEQ ID NO: 469 - DNA, 354 bp, Synthetic construct
  source 1..354: mol_type = other DNA
caggtgcagctggtggagtctggggggggcgtggtccagcctgggaggtccctgagactctcctgtgcagcgtctggattcaccttcagtagctatggcatgcactgggtccgccagactccaggcaaggggctggagtgggtggcagttatatactatgatggaaataataaatactatgcagactccgtgaagggccgattcaccatctccagagagaattccaagaacacgctgtatctgcaaatgaacagcctgagagccgatgacacggctctatattactgtgcgagagaactaactggggatgcttttgatctctggggccaagggacaatggtcaccgtctcttca

SEQ ID NO: 470 - DNA, 321 bp, Synthetic construct
  source 1..321: mol_type = other DNA
gaaattgtgctgactcagtctccagattttcagtctgcgactccaaaggagaaagtcaccatcacctgccgggccagtcagagcattggtagtcacttacactggtatcagcagaaaccagatcagtctccaaagctcctcatcaagtatgcttcccagtccttctcaggggtcccctcgaggttcagtggcagtggatctgggacagatttcaccctcaccatcaatagactggaagatgaagatgctgcaacgtattactgtcatcagagtagtaggttcccgtacacttttggccaggggaccaagctggatatcaaa

SEQ ID NO: 471 - protein, 668 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 472 - protein, 699 aa, Synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGSGLNDIFEAQKIEWHEGGGGSHHHHHH

SEQ ID NO: 473 - protein, 391 aa, Synthetic construct
QYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGSGLNDIFEAQKIEWHEGGGGSHHHHHH

SEQ ID NO: 474 - protein, 342 aa, Synthetic construct
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAGGGGSGLNDIFEAQKIEWHEGGGGSHHHHHH

SEQ ID NO: 475 - protein, 5 aa, Synthetic construct
GYYIH

SEQ ID NO: 476 - protein, 17 aa, Synthetic construct
WINPNSGGTNYAQKFQG

SEQ ID NO: 477 - protein, 9 aa, Synthetic construct
EDTWNYIDY

SEQ ID NO: 478 - protein, 11 aa, Synthetic construct
RASQSISSHLN

SEQ ID NO: 479 - protein, 7 aa, Synthetic construct
AASSLHS

SEQ ID NO: 480 - protein, 9 aa, Synthetic construct
QQSYSTPRT

SEQ ID NO: 481 - protein, 118 aa, Synthetic construct
QVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSS

SEQ ID NO: 482 - protein, 107 aa, Synthetic construct
DIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIK

SEQ ID NO: 483 - protein, 447 aa, Synthetic construct
QVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 484 - protein, 214 aa, Synthetic construct
DIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 485 - protein, 354 aa, Synthetic construct
CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACTATATACACTGGGTGCGACAGGCCCCTGGACAAGGGCTTGAGTGGATGGGATGGATCAACCCTAACAGTGGTGGCACAAACTATGCACAGAAGTTTCAGGGCAGGGTCACCATGACCAGGGACACGTCCATCGGCACAGCCTACATGGAGCTGAGCAGGCTGAGATCTGACGACACGGCCATTTATTACTGTACGAGAGAGGACACCTGGAACTACATTGACTACTGGGGCCAGGGAACCCTGGTCACCGTCTCCTCA

SEQ ID NO: 486 - protein, 321 aa, Synthetic construct
GACATCCAGATGACCCAGTCTCCATCCTCCCTGTCTGCATCTGTAGGAGACAGAGTCACCATCACTTGCCGGGCAAGTCAGAGCATTAGCAGCCATTTAAATTGGTATCAGCAGAAACCAGGGAAAGCCCCTAAACTCCTGATCTATGCTGCATCCAGTTTACACAGTGGGGTCCCATCAAGGTTCAGTGGCAGTGGATCTGGGACAGATTTCACTCTCACCATCAGCAGTCTGCAACCTGAAGATTTTGCAACTTACTGCTGTCAACAGAGTTACAGTACCCCTCGGACGTTCGGCCAGGGGACCAAGGTGGAAATCAAA

SEQ ID NO: 487 - protein, 1341 aa, Synthetic construct
CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACTATATACACTGGGTGCGACAGGCCCCTGGACAAGGGCTTGAGTGGATGGGATGGATCAACCCTAACAGTGGTGGCACAAACTATGCACAGAAGTTTCAGGGCAGGGTCACCATGACCAGGGACACGTCCATCGGCACAGCCTACATGGAGCTGAGCAGGCTGAGATCTGACGACACGGCCATTTATTACTGTACGAGAGAGGACACCTGGAACTACATTGACTACTGGGGCCAGGGAACCCTGGTCACCGTCTCCTCAGCTAGCACTAAAGGGCCTTCTGTATTTCCCTTGGCCCCGTCCAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAATGTGAACCACAAACCTTCAAATACAAAAGTGGATAAACGCGTAGAACCGAAATCGTGTGATAAAACTCACACATGCCCGCCATGCCCGGCACCTGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCACGAAGATCCCGAAGTTAAGTTCAATTGGTATGTTGATGGTGTAGAGGTACACAATGCTAAGACTAAACCTCGCGAGGAGCAGTACAATTCGACCTATCGTGTCGTGAGCGTTCTGACCGTCCTTCACCAAGATTGGCTTAACGGCAAAGAATATAAGTGCAAGGTAAGCAATAAAGCACTTCCGGCCCCAATCGAGAAAACCATTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTAAAGGTTTTTATCCTAGCGATATTGCTGTTGAATGGGAATCGAACGGTCAGCCGGAGAATAATTATAAAACAACGCCACCCGTCCTGGATAGCGACGGCTCATTTTTTCTGTATAGCAAACTGACTGTAGATAAATCACGGTGGCAGCAGGGCAATGTATTCAGTTGCTCCGTTATGCATGAAGCGTTACATAATCACTACACGCAGAAATCTCTTAGTCTTTCACCCGGT

SEQ ID NO: 488 - protein, 642 aa, Synthetic construct
GACATCCAGATGACCCAGTCTCCATCCTCCCTGTCTGCATCTGTAGGAGACAGAGTCACCATCACTTGCCGGGCAAGTCAGAGCATTAGCAGCCATTTAAATTGGTATCAGCAGAAACCAGGGAAAGCCCCTAAACTCCTGATCTATGCTGCATCCAGTTTACACAGTGGGGTCCCATCAAGGTTCAGTGGCAGTGGATCTGGGACAGATTTCACTCTCACCATCAGCAGTCTGCAACCTGAAGATTTTGCAACTTACTGCTGTCAACAGAGTTACAGTACCCCTCGGACGTTCGGCCAGGGGACCAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCTGAATAATTTTTATCCGCGTGAGGCTAAAGTACAATGGAAAGTCGACAATGCTTTGCAGTCGGGAAATTCACAGGAAAGTGTTACGGAGCAGGATTCTAAAGATTCCACATATTCACTCAGCTCCACCCTTACACTGAGCAAAGCCGACTATGAAAAACATAAAGTTTACGCATGTGAGGTGACGCACCAAGGATTATCCAGTCCGGTCACAAAATCGTTTAACCGCGGTGAGTGT

SEQ ID NO: 489 - protein, 7 aa, Synthetic construct
TSGMCVS

SEQ ID NO: 490 - protein, 16 aa, Synthetic construct
LIDWDGDNYYSTSLKT

SEQ ID NO: 491 - protein, 12 aa, Synthetic construct
MGYTYGYDAFDI

SEQ ID NO: 492 - protein, 11 aa, Synthetic construct
RASQSIGSLLH

SEQ ID NO: 493 - protein, 7 aa, Synthetic construct
FASQSLS

SEQ ID NO: 494 - protein, 9 aa, Synthetic construct
HQSRSLPFT

SEQ ID NO: 495 - protein, 122 aa, Synthetic construct
QVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSS

SEQ ID NO: 496 - protein, 107 aa, Synthetic construct
EIVLTQSPEFQSVTPTEKVTITCRASQSIGSLLHWYQQKPDQSPKLLIKFASQSLSGVPSRFSGSGSGTDFTLTINSLEAEDAATYYCHQSRSLPFTFGPGTKVDIN

SEQ ID NO: 497 - protein, 451 aa, Synthetic construct
QVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 498 - protein, 214 aa, Synthetic construct
EIVLTQSPEFQSVTPTEKVTITCRASQSIGSLLHWYQQKPDQSPKLLIKFASQSLSGVPSRFSGSGSGTDFTLTINSLEAEDAATYYCHQSRSLPFTFGPGTKVDINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 499 - DNA, 366 bp, Synthetic construct
  source 1..366: mol_type = other DNA
caggtcaccttgagggagtctggtcctgcgctggtgaaacccacacagaccctcacactgacctgcaccttctctgggatctcacttaccactagtggaatgtgtgtgagttggatccgtcagcccccagggaaggccctggagtggcttgcactcattgattgggatggtgataactactacagcacatctctgaagaccaggctcaccatctccaaggacacctccaaaaaccaggtggtccttacaatggccagcatggaccctgtggacacagccacgtattactgtgcacggatgggatacacctatggttacgatgcttttgatatctggggccaagggacaatggtcaccgtctcttca

SEQ ID NO: 500 - DNA, 321 bp, Synthetic construct
  source 1..321: mol_type = other DNA
gaaattgtgctgactcagtctccagaatttcagtctgtgactccaacggagaaagtcaccatcacctgccgggccagtcagagcattggtagtctcttacactggtaccagcagaaaccagatcagtctccaaagctcctcatcaagtttgcttcccagtccctctcaggggtcccctcgaggttcagtggcagtggatctgggacagatttcaccctcaccatcaatagcctggaagctgaagatgctgcaacgtattactgtcatcagagtaggagtttaccattcactttcggccctgggaccaaagtggatatcaat

SEQ ID NO: 501 - DNA, 1353 bp, Synthetic construct
  source 1..1353: mol_type = other DNA
caggtcaccttgagggagtctggtcctgcgctggtgaaacccacacagaccctcacactgacctgcaccttctctgggatctcacttaccactagtggaatgtgtgtgagttggatccgtcagcccccagggaaggccctggagtggcttgcactcattgattgggatggtgataactactacagcacatctctgaagaccaggctcaccatctccaaggacacctccaaaaaccaggtggtccttacaatggccagcatggaccctgtggacacagccacgtattactgtgcacggatgggatacacctatggttacgatgcttttgatatctggggccaagggacaatggtcaccgtctcttcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 502 - DNA, 642 bp, Synthetic construct
  source 1..642: mol_type = other DNA
gaaattgtgctgactcagtctccagaatttcagtctgtgactccaacggagaaagtcaccatcacctgccgggccagtcagagcattggtagtctcttacactggtaccagcagaaaccagatcagtctccaaagctcctcatcaagtttgcttcccagtccctctcaggggtcccctcgaggttcagtggcagtggatctgggacagatttcaccctcaccatcaatagcctggaagctgaagatgctgcaacgtattactgtcatcagagtaggagtttaccattcactttcggccctgggaccaaagtggatatcaatcgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 503 - protein, 7 aa, Synthetic construct
TSGMCVS

SEQ ID NO: 504 - protein, 16 aa, Synthetic construct
LIDWDDDKYYSTSLKT

SEQ ID NO: 505 - protein, 12 aa, Synthetic construct
IRYIYGYDYFDY

SEQ ID NO: 506 - protein, 11 aa, Synthetic construct
RASQRISSQLN

SEQ ID NO: 507 - protein, 7 aa, Synthetic construct
AASSLQS

SEQ ID NO: 508 - protein, 9 aa, Synthetic construct
QQTYSKPIT

SEQ ID NO: 509 - protein, 122 aa, Synthetic construct
QVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSS

SEQ ID NO: 510 - protein, 107 aa, Synthetic construct
DIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIK

SEQ ID NO: 511 - protein, 451 aa, Synthetic construct
QVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 512 - protein, 214 aa, Synthetic construct
DIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 513 - protein, 366 aa, Synthetic construct
CAGGTCACCTTGAGGGAGTCTGGTCCTGCGCTGGTGAAAACCACACAGACCCTCACACTGACCTGCACCTTCTCTGGGTTCTCACTCAGCACTAGTGGAATGTGTGTGAGCTGGATCCGTCAGCCCCCAGGGAAGGCCCTGGAGTGGCTTGCACTCATTGATTGGGATGATGATAAATACTACAGCACATCTCTGAAGACCAGGCTCACCATCTCCAAGGACACCTCCAAATCCCAGGTGGTCCTTACAATGACCAGCATGGACCCTGTGGACACAGCCACGTATTACTGTGCACGGATACGATACATCTATGGTTACGACTACTTTGACTACTGGGGCCAGGGAACCCTGGTCACCGTCTCCTCA

SEQ ID NO: 514 - protein, 107 aa, Synthetic construct
EIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIK

SEQ ID NO: 515 - protein, 21 aa, Synthetic construct
MALPVTALLLPLALLLHAARP

SEQ ID NO: 516 - DNA, 140 bp, Synthetic construct
  source 1..140: mol_type = other DNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 517 - DNA, 100 bp, Synthetic construct
  source 1..100: mol_type = other DNA
cgggtggcatccctgtgacccctccccagtgcctctcctggccctggaagttgccactccagtgcccaccagccttgtcctaataaaattaagttgcatc

SEQ ID NO: 518 - DNA, 48 bp, Synthetic construct
  source 1..48: mol_type = other DNA
gggtcaagattagagaacggtcgtagcattatcggaggttctgccacc

SEQ ID NO: 519 - DNA, 59 bp, Synthetic construct
  source 1..59: mol_type = other DNA
ggcacatttgaataaattctattaccagttcaagcttggcgtaatcatggtcatagctg

SEQ ID NO: 520 - protein, 22 aa, Synthetic construct
MDMRVPAQLLGLLLLWLPGAKC

SEQ ID NO: 521 - protein, 93 aa, Homo sapiens
LSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 522 - protein, 66 aa, Homo sapiens
IEVMYPPPYLDNERSNGTIIHVKGKHLCPSPLFPGPSKPFWVLVVVGGVLACYSLLVTVAFIIFWV

SEQ ID NO: 523 - protein, 118 aa, Synthetic construct; identical to SEQ ID NO: 433

SEQ ID NO: 524 - protein, 107 aa, Synthetic construct; identical to SEQ ID NO: 514

SEQ ID NO: 525 - protein, 118 aa, Synthetic construct; identical to SEQ ID NO: 481

SEQ ID NO: 526 - protein, 107 aa, Synthetic construct; identical to SEQ ID NO: 482

SEQ ID NO: 527 - protein, 122 aa, Synthetic construct; identical to SEQ ID NO: 495

SEQ ID NO: 528 - protein, 107 aa, Synthetic construct
EIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDIN

SEQ ID NO: 529 - protein, 122 aa, Synthetic construct; identical to SEQ ID NO: 509

SEQ ID NO: 530 - protein, 107 aa, Synthetic construct; identical to SEQ ID NO: 510

SEQ ID NO: 531 - protein, 115 aa, Synthetic construct
QVQLVQSGAEVKKPGASVKVSCKASGYTFTGYYMHWVRQAPGQGLEWMGWVDPKNGDTNYAHKFQGRVTMIWDTSISTAYMELSRLISDDTAVYYCARDADFDYWGQGTLVTVSS

SEQ ID NO: 532 - protein, 112 aa, Synthetic construct
DVVMTQTPSSLSASLGDRVTISCRASQSLVYIDGNTYLNWFRQRPGQSPRRLIYKVSNRDYGVPDRFSGSGSGTDFTLKISRVEAEDVGVYYCMQGTHWPLTFGGGTKVEIK

SEQ ID NO: 533 - protein, 112 aa, Synthetic construct
DVVMTQSPLSLPVTLGQPASISCRSSQSLVYIDGNTYLNWFRQRPGQSPRRLIYKVSNRDYGVPDRFSGSGSGTDFTLKISRVEAEDVGVYYCMQGTHWPLTFGGGTKVEIK

SEQ ID NO: 534 - protein, 107 aa, Synthetic construct; identical to SEQ ID NO: 434

SEQ ID NO: 535 - protein, 107 aa, Synthetic construct; identical to SEQ ID NO: 496

SEQ ID NO: 536 - protein, 124 aa, Synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 537 - protein, 107 aa, Synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 538 - protein, 73 aa, Homo sapiens
TTTPAPRPPTPAPTIASQPLSLRPEACRPAAGGAVHTRGLDFACDIYIWAPLAGTCGVLLLSLVITLYCNHRN

SEQ ID NO: 539 - protein, 53 aa, Homo sapiens
SLRPEACRPAAGGAVHTRGLDFACDIYIWAPLAGTCGVLLLSLVITLYCNHRN

SEQ ID NO: 540 - protein, 93 aa, Homo sapiens; identical to SEQ ID NO: 521

SEQ ID NO: 541 - protein, 40 aa, Homo sapiens
IEVMYPPPYLDNEKSNGTIIHVKGKHLCPSPLFPGPSKPF

SEQ ID NO: 542 - protein, 26 aa, Homo sapiens
WVLVVVGGVLACYSLLVTVAFIIFWV

SEQ ID NO: 543 - protein, 41 aa, Homo sapiens
RSKRSRLLHSDYMNMTPRRPGPTRKHYQPYAPPRDFAAYRS

SEQ ID NO: 544 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 545 - protein, 489 aa, Synthetic construct
MDMRVPAQLLGLLLLWLPGAKCQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 546 - protein, 476 aa, Synthetic construct
MDMRVPAQLLGLLLLWLPGAKCQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 547 - protein, 496 aa, Synthetic construct
MDMRVPAQLLGLLLLWLPGAKCQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 548 - protein, 516 aa, Synthetic construct
MDMRVPAQLLGLLLLWLPGAKCQVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDAFDIWGQGTKVTVSSGGGGSGGGGSGGGGSGGGGSEIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIKASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 549 - protein, 489 aa, Synthetic construct
MDMRVPAQLLGLLLLWLPGAKCQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 550 - protein, 476 aa, Synthetic construct
MDMRVPAQLLGLLLLWLPGAKCQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 551 - protein, 496 aa, Synthetic construct
MDMRVPAQLLGLLLLWLPGAKCQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 552 - protein, 516 aa, Synthetic construct
MDMRVPAQLLGLLLLWLPGAKCQVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTWNYIDYWGQGTLVTVSSGGGGSGGGGSGGGGSGGGGSDIQMTQSPSSLSASVGDRVTITCRASQSISSHLNWYQQKPGKAPKLLIYAASSLHSGVPSRFSGSGSGTDFTLTISSLQPEDFATYCCQQSYSTPRTFGQGTKVEIKASTTTSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 553 - protein, 493 aa, Synthetic construct
MDMRVPAQLLGLLLLWLPGAKCQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 554 - protein, 480 aa, Synthetic construct
MDMRVPAQLLGLLLLWLPGAKCQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 555 - protein, 500 aa, Synthetic construct
MDMRVPAQLLGLLLLWLPGAKCQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 556 - protein, 520 aa, Synthetic construct
MDMRVPAQLLGLLLLWLPGAKCQVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYGYDAFDIWGQGTMVTVSSGGGGSGGGGSGGGGSGGGGSEIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDINASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 557 - protein, 493 aa, Synthetic construct
MDMRVPAQLLGLLLLWLPGAKCQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 558 - protein, 480 aa, Synthetic construct
MDMRVPAQLLGLLLLWLPGAKCQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 559 - protein, 500 aa, Synthetic construct
MDMRVPAQLLGLLLLWLPGAKCQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 560 - protein, 520 aa, Synthetic construct
MDMRVPAQLLGLLLLWLPGAKCQVTLRESGPALVKTTQTLTLTCTFSGFSLSTSGMCVSWIRQPPGKALEWLALIDWDDDKYYSTSLKTRLTISKDTSKSQVVLTMTSMDPVDTATYYCARIRYIYGYDYFDYWGQGTLVTVSSGGGGSGGGGSGGGGSGGGGSDIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 561 - protein, 495 aa, Synthetic construct
MDMRVPAQLLGLLLLWLPGAKCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 562 - protein, 482 aa, Synthetic construct
MDMRVPAQLLGLLLLWLPGAKCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 563 - protein, 502 aa, Synthetic construct
MDMRVPAQLLGLLLLWLPGAKCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 564 - protein, 522 aa, Synthetic construct
MDMRVPAQLLGLLLLWLPGAKCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKASLSNSIMYFSHFVPVFLPAKPTTTPAPRPPTPAPTIASQPLSLRPEACRPAAGGAVHTRGLDFACDIYIWAPLAGTCGVLLLSLVITLYCNHRNVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 565 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 566 - protein, 10 aa, Synthetic construct
GFTFRSFGMS

SEQ ID NO: 567 - protein, 10 aa, Synthetic construct
SISGSGSDTL

SEQ ID NO: 568 - protein, 6 aa, Synthetic construct
GGSLSR

SEQ ID NO: 569 - protein, 98 aa, Synthetic construct
QVQLVESGGGLVQPGGSLRLSCAASGFTLDYYAIGWFRQAPGKEREGVSCISSSDGSTYYADSVKGRFTISRDNAKNTVYLQMNSLKPEDTAVYYCAA

SEQ ID NO: 570 - protein, 235 aa, Homo sapiens; identical to SEQ ID NO: 565

SEQ ID NO: 571 - protein, 10 aa, synthetic construct
  VARIANT 5: note = X is selected from D and E
GFTFXDYAIG

SEQ ID NO: 572 - protein, 10 aa, synthetic construct
  VARIANT 7: note = X is selected from G and E
  VARIANT 8: note = X is selected from N and Q
CIRTYDXXTY

SEQ ID NO: 573 - protein, 23 aa, synthetic construct
  VARIANT 8: note = X is selected from K, E and Y
  VARIANT 17: note = X is selected from N and G
  VARIANT 21: note = X is selected from M and L
GSYYACAXYSRPDPSEXHVDXDY

SEQ ID NO: 574 - protein, 17 aa, synthetic construct
  VARIANT 7: note = X is selected from G and E
  VARIANT 8: note = X is selected from N and Q
  VARIANT 12: note = X is selected from I and A
CIRTYDXXTYYXDSVKG

(19) United States
(12) Patent Application Publication
(10) Pub. No.: US 2026/0306659 A1
(43) Pub. Date: Oct. 8, 2026

(54) LIPID NANOPARTICLES AND METHODS OF MANUFACTURE AND USE THEREOF

(71) Applicants: Genzyme Corporation, Cambridge, MA (US); Ablynx N.V., Ghent-Zwijnaarde (BE)

(72) Inventors: Mir ALI, Woburn, MA (US); Pilar BATALLA BOSQUET, Ghent-Zwijnaarde (BE); Piotr BOBROWICZ, Hanover, NH (US); Austin Wayne BOESCH, Somerville, MA (US); Lieselotte CRUL, Ghent-Zwijnaarde (BE); Francis DESCAMPS, Ghent-Zwijnaarde (BE); Daryl Clark DRUMMOND, Lincoln, MA (US); Aaron GRISET, Somerville, MA (US); Sara Marie HALMOS, Waltham, MA (US); William KUHLMAN, Somerville, MA (US); Viktor LEMGART, Boston, MA (US); Olga LIHORADOVA, Cambridge, MA (US); Zhiquian Lucy LIU, Waltham, MA (US); Ulrik NIELSEN, Quincy, MA (US); Andrew SAWYER, North Reading, MA (US); Jurgen VAN IMPE, Ghent-Zwijnaarde (BE)

(21) Appl. No.: 19/706,763

(22) Filed: Jun. 12, 2026

Related U.S. Application Data
(63) Continuation of application No. 19/284,535, filed on Jul. 29, 2025.
(60) Provisional application No. 63/677,381, filed on Jul. 30, 2024.
(60) Provisional application No. 63/786,916, filed on Apr. 10, 2025.

(51) Int. Cl. A61K 47/69; A61K 31/7105; A61K 47/68; C07K 16/28

(52) U.S. Cl. CPC A61K 47/6913; A61K 31/7105; A61K 47/6849; A61K 47/6889; C07K 16/2803; C07K 16/2815; A61K 2239/17; A61K 2239/21; C07K 2317/565; C07K 2317/567; C07K 2317/569; C07K 2317/622

(57) ABSTRACT

The present disclosure relates generally to compositions and methods for gene therapy, and more specifically to delivering mRNA-based therapeutics to immune cells in vivo.

### CROSS REFERENCE TO RELATED APPLICATIONS

This application is a continuation of U.S. patent application Ser. No. 19/284,535, filed Jul. 29, 2025, which claims priority benefit to U.S. Provisional Application No. 63/677,381, filed Jul. 30, 2024, and U.S. Provisional Application No. 63/786,916, filed Apr. 10, 2025, the disclosures of each of which are hereby incorporated herein by reference in their entireties for all purposes.

### REFERENCE TO AN ELECTRONIC SEQUENCE LISTING

The content of the electronic sequence listing (183952035401SEQLIST.xml; Size: 1,376,904 bytes; and Date of Creation: Jun. 4, 2026) is herein incorporated by reference in its entirety.

### FIELD

The present disclosure relates generally to compositions and methods for gene therapy, and more specifically to delivering mRNA-based therapeutics to immune cells in vivo.

### BACKGROUND

Genetic modification of T-cells with chimeric antigen receptors (CARs) to target specific diseases has shown impressive clinical responses in patients with hematologic malignancies [1]. However, several barriers remain before this therapy is available to a broader patient population. Currently, CAR-T cell therapy production is carried out ex vivo, including genetic modification of the patient's T-cells in culture before infusing the cells back into the patient [2]. The ex vivo methods required to generate sufficient numbers of tumor-specific T-cells are complex thereby hindering widespread application to treat cancer patients [3]. Additionally, two CAR-T therapies approved by the U.S. Food and Drug Administration (FDA) are priced at US$373,000 (Yescarta) and US$475,000 (Kymriah), making it economically challenging to provide this personalized treatment to a broader array of cancer patients worldwide [4]. Finally, CAR T-cell therapy results in acute and chronic toxicities that limit its overall therapeutic index in cancer patients [5]. The onset of immune activation, known as cytokine release syndrome (CRS), is the most prevalent adverse effect following CAR T-cell infusion. Additionally, other side effects reported in patients receiving CAR T-cells include the development of neurological toxicities, on-target/off-tumor recognition, anaphylaxis, and insertional oncogenesis [6-8]. In order to overcome these significant challenges and limitations, more innovative strategies are required to program T-cells to express CARs.

Particularly, Ex vivo chimeric antigen receptor (CAR) T-cell therapy has proven successful in patients with B-cell hematologic malignancies. However, its broad application still faces significant challenges due to current approaches requiring elaborate and expensive techniques to engineer and manufacture T-cells. Additionally, barriers such as limited efficacy against solid tumors, treatment-associated toxicities, lack of CAR-T cell trafficking to the tumor microenvironment, on-target off-tumor effects, and tumor antigen escape must be overcome.

In-vitro transcribed (IVT) mRNA has, in recent years, proven an effective technology to express therapeutic proteins and antigens in cells, making it a promising alternative to DNA-based products. By utilizing IVT mRNA, researchers allow for transient protein expression without causing integration into the genome [9-11], and do not require nuclear import or slow cytoplasmic diffusion [12]. However, to function in vivo, effective and stable delivery platforms, protecting the mRNA from degradation and allowing for specific target cell uptake, are required [13]. One such delivery system that has entered the clinic and proven successful is lipid nanoparticles (LNPs), employing an ionizable cationic lipid to condense nucleic acids into a relatively uniform solid lipid nanoparticle approximating 100 nm in diameter [14-16]. Remarkably, the recent authorization of two coronavirus (COVID-19) vaccines [17, 18] utilizing LNPs to deliver mRNA intramuscularly stands out as noticeable examples. However, to date, the use and design of LNPs for systemic delivery have primarily allowed for cellular uptake by hepatocytes and Kupffer cells of the liver [19, 20]. To solve this, the advantages of adding targeting ligands to nanoparticles and thereby guiding the specificity and delivery of a nucleic acid payload toward a cell type of interest have been shown [21-33].

Thus, what is needed is a delivery platform that overcomes some of the barriers in existing T-cell therapies. More particularly, what is needed is a delivery platform that overcomes some of the barriers in existing CAR T-cell therapies for B-cell hematologic malignancies.

### BRIEF SUMMARY

In one aspect, provided herein is a lipid nanoparticle (LNP) comprising: (a) a lipid-immune cell targeting group conjugate comprising the compound of Formula (II): [Lipid]-[optional linker]-[antibody], (b) an ionizable cationic lipid, and (c) a nucleic acid wherein the nucleic acid is encapsulated in the LNP. In some embodiments, the antibody is an immunoglobulin single variable domain (ISVD) that specifically binds to human CD8alpha. In some embodiments, the ISVD comprises complementarity-determining regions 1 (CDR1), 2 (CDR2), and 3 (CDR3) of an ISVD having the sequence selected from the group consisting of SEQ ID NOs: 160 to 179. In some embodiments, the antibody is an ISVD that specifically binds to human CD8alpha, and the nucleic acid encodes a polypeptide comprising CD22-chimeric antigen receptor (CAR).

In another aspect, provided herein is an immunoglobulin single variable domain (ISVD) specifically binding human CD8alpha. In some embodiments, the ISVD essentially consists of 4 framework regions (FR1 to FR4, respectively) and 3 complementarity determining regions (CDR1 to CDR3, respectively). In some embodiments, CDR1 (according to AbM definition) has an amino acid sequence selected from the group consisting of: the amino acid sequence of SEQ ID NO: 244; amino acid sequences that have at least 80% amino acid identity with the amino acid sequence of SEQ ID NO: 244; and amino acid sequences that have 3, 2, or 1 amino acid difference with the amino acid sequences of SEQ ID NO: 244. In some embodiments, CDR2 (according to AbM definition) has an amino acid sequence selected from the group consisting of: the amino acid sequence of SEQ ID NO: 246; amino acid sequences that have at least 80% amino acid identity with the amino acid sequence of SEQ ID NO: 246; and amino acid sequences that have 3, 2, or 1 amino acid difference with the amino acid sequence of SEQ ID NO: 246. In some embodiments, CDR3 (according to AbM definition) has an amino acid sequence selected from the group consisting of: the amino acid sequence of SEQ ID NO: 248; amino acid sequences that have at least 80% amino acid identity with the amino acid sequence of SEQ ID NO: 248; and amino acid sequences that have 3, 2, or 1 amino acid difference with the amino acid sequence of SEQ ID NO: 248.

In another aspect, provided herein is a conjugate comprising an ISVD linked to a phospholipid-PEG-maleimide derivative.

In another aspect, provided herein is a method for the preparation of a composition comprising monomers of an ISVD with a cysteine containing linker at its C-terminal end. In some embodiments, the method comprises the following sequential steps: (a) reducing a composition comprising ISVD dimers to ISVD monomers with a first reducing agent, wherein the ISVD dimers are formed through the cysteine containing linker at the C-terminal end of the ISVD; (b) purifying the ISVD monomers obtained in step (a) to get a purified composition comprising the ISVD monomers; (c) reducing the purified composition obtained in step (b) with a second reducing agent; and (d) purifying the reduced composition obtained in step (c) to obtain a composition comprising monomers of the ISVD.

In another aspect, provided herein is a method for the preparation of a phospholipid-PEG-ISVD conjugate. In some embodiments, the method comprises the following sequential steps: (a) mixing a first composition comprising monomers of an ISVD comprising a cysteine containing linker, with a second composition comprising phospholipid-PEG molecules comprising a bioconjugation linker under conditions that the phospholipid-PEG molecules and the ISVD monomers can form a conjugate through clicking chemistry; and (b) adding cysteine to the conjugate obtained in step (a) under conditions that the conjugation reaction is quenched, wherein a composition comprising the phospholipid-PEG ISVD conjugate is obtained.

In one aspect, provided herein is a novel delivery platform, employing targeted lipid nanoparticles (LNPs) encapsulating CD22 CAR-encoding mRNA to reprogram circulating human T-cells in vivo, thus providing a strategy for overcoming some of these barriers. In some embodiments, the approach can be utilized to deliver mRNA encoding a novel CD22 CAR specifically to CD8⁺ T-cell using an immunoglobulin single variable domain (ISVD)-based targeting moiety, thereby enabling transient functional CAR expression in vitro and in vivo. In some embodiments, the targeted LNP formulation allows for repeated dosing strategies while minimizing off-target cell mRNA expression. In some embodiments, the in vivo reprogramming of non-stimulated T-cells to express a CD22 CAR mediates tumor cell growth inhibition in a humanized Nalm6 cancer mouse model.

In one aspect, provided herein is a novel approach to selectively reprogram human CD8+ T-cells in vivo by delivering IVT mRNA via antibody-targeted LNPs. In some embodiments, the clinical relevance of the technology is shown by specifically delivering mRNA encoding a novel CD22 CAR to T-cells in vivo. In some embodiments, functional CAR-mediated cancer cell killing in a humanized mouse model is reported. In some embodiments, de-targeting of the liver and lung, as well as off-target immune cell populations in the blood, is achieved through careful engineering of the surface charge, pegylation strategy, and particle size, allowing for relatively specific transfection of CD8+ T-cells. In some embodiments, the tolerability is improved through decrease in undesirable cytokine responses via meticulous design and selection of the ionizable lipid, targeting ligand, and CAR sequence, and by limiting the presence of mRNA impurities, including double-stranded mRNA.

The present disclosure provides lipid nanoparticles (LNPs). In some embodiments, the LNPs comprise a lipid-immune cell targeting group conjugate comprising (a) the compound of Formula (II): [Lipid]-[optional linker]-[antibody]. In some embodiments, the LNPs further comprise (b) an ionizable cationic lipid. In some embodiments, the LNPs further comprise (c) a nucleic acid, wherein the nucleic acid is encapsulated in the LNP. In some embodiments, the antibody is an immunoglobulin single variable domain (ISVD) that specifically binds to human CD8alpha. In some embodiments, the ISVD comprises complementarity-determining regions 1 (CDR1), 2 (CDR2), and 3 (CDR3) of an ISVD having the sequence selected from the group consisting of SEQ ID NOs: 160 to 179. In some embodiments, the antibody is an ISVD that specifically binds to human CD8alpha, and the nucleic acid encodes a polypeptide comprising CD22-chimeric antigen receptor (CAR). In some embodiments, the ISVD comprises complementarity-determining regions 1 (CDR1), 2 (CDR2), and 3 (CDR3) of an ISVD having the sequence selected from the group consisting of SEQ ID NOs: 160 to 179, and the nucleic acid encodes a polypeptide comprising CD22-chimeric antigen receptor (CAR).

In some embodiments, the ISVD specifically binding to CD8alpha comprises CDR1, CDR2, and CDR3 according to the Abm CDR definition, and CDR1 is chosen from the group consisting of: (i) SEQ ID NO: 244; and (ii) amino acid sequences that have 3, 2, or 1 amino acid difference with at least one of the amino acid sequences of SEQ ID NO: 244. In some embodiments, CDR2 is chosen from the group consisting of: (i) SEQ ID NO: 246; and (ii) amino acid sequences that have 3, 2, or 1 amino acid difference with at least one of the amino acid sequences of SEQ ID NO: 246. In some embodiments, CDR3 is chosen from the group consisting of (i) SEQ ID NO: 248 and (ii) amino acid sequences that have 3, 2, or 1 amino acid difference with at least one of the amino acid sequences of SEQ ID NO: 248.

In some embodiments, the antibody of the LNPs specifically binds to human CD8alpha is covalently coupled to the Lipid in Formula (II) via a linker comprising polyethylene glycol (PEG). In some embodiments, the Lipid in Formula (II) covalently coupled to the antibody is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

In some embodiments, the Lipid in Formula (II) covalently coupled to the antibody is DSPE. In some embodiments, the PEG has a molecular weight of about 1 k Daltons to about 5 k Daltons. In some embodiments, the PEG is PEG 3400 (PEG 3.4K).

In some embodiments, the immunoglobulin single variable domain comprises SEQ ID NO: 9, SEQ ID NO: 169 or SEQ ID NO: 44, or a sequence having at least 85%, at least 90%, at least 95%, at least 99% identity to SEQ ID NO: 9, SEQ ID NO: 169 or SEQ ID NO: 44.

In some embodiments, the LNPs further comprise a structural lipid, a neutral phospholipid, or a free PEG-lipid, or any combination thereof.

In some embodiments, the structural lipid comprises or is sterol. In some embodiments, the sterol comprises or is cholesterol. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin. In some embodiments, the neutral phospholipid comprises or is DSPC.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the free PEG lipid is PEG-dioleoylgylcerol (PEG-DOG), PEG-dimyristoyl-glycerol (PEG-DMG), PEG-dipalmitoyl-glycerol (PEG-DPG), PEG-(PEG-DLPE), PEG-dimyrstoyl-dilinoleoyl-glycero-phosphatidyl ethanolamine phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]—N,N-ditetradecylacetamide, diacylphosphatidylethanolamine comprising Dipalmitoyl (C16) chain or Distearoyl (C18) chain, or a PEG-distearoyl-phosphatidylethanolamine (PEG-DSPE) lipid. In some embodiments, the PEG-lipid comprises PEG-DMG, PEG-DPG, or PEG-DSG, or any combination thereof. In some embodiments, free PEG-lipid comprises PEG-DPG. In some embodiments, the PEG-DPG comprises or is PEG 2000-DPG (DPG-PEG 2000).

In some embodiments, the nucleic acid comprises or is RNA. In some embodiments, the RNA comprises or is mRNA. In some embodiments, the mRNA encodes a synthetic T cell receptor (synTCR) or a Chimeric Antigen Receptor (CAR). In some embodiments, the mRNA comprises a 5′ Cap, a 5′ untranslated region (UTR), a sequence encoding a polypeptide, a 3′ UTR, and optionally a polyA tail.

In some embodiments, the nucleic acid comprises (1) optionally, a 5′ cap; (2) optionally, a 5′ UTR region; (3) optionally, nucleotides encoding a Lead peptide sequence; (4) nucleotides encoding an antibody heavy chain variable region (VH); (5) optionally, nucleotides encoding a Linker A; (6) nucleotides encoding an antibody light chain variable region (VL); (7) nucleotides encoding a Linker B, (8) nucleotides encoding a Hinge domain; (9) nucleotides encoding a Transmembrane domain; (10) nucleotides encoding a Co-stimulatory domain; (11) nucleotides encoding a Signaling domain; (12) optionally, a 3′ UTR region; and (13) optionally, a polyA tail.

In some embodiments, the nucleic acid comprises the following formula, arranged from 5′ to 3′: 5′ UTR (optional)-nucleotides encoding the Lead peptide sequence (optional)-nucleotides encoding the antibody heavy chain variable region (VH)-nucleotides encoding the Linker A (optional)-nucleotides encoding the antibody light chain variable region (VL)-nucleotides encoding Linker B (optional)-nucleotides encoding the Hinge-nucleotides encoding the Transmembrane domain-nucleotides encoding the Co-stimulatory domain-nucleotides encoding the Signaling domain—3′ UTR (optional).

In some embodiments, the polypeptide encoded by the nucleic acid comprises an antibody specifically binding to B-cell, a Hinge domain and a Transmembrane domain (Hinge and Transmembrane domains), a Co-stimulatory domain, and a Signaling domain.

In some embodiments, the polypeptide encoded by the nucleic acid comprises the following formula, arranged from N-terminus to C-terminus: [Lead peptide sequence (optional)]-[antibody specifically binding to B-cell]-[Linker B (optional)]-[Hinge domain]-[Transmembrane domain]-[Co-stimulatory domain]-[Signaling domain].

In some embodiments, the optional Lead peptide sequence comprises a signal peptide. In some embodiments, the signal peptide is derived from CD8 (SEQ ID NO: 565). In some embodiments, the signal peptide comprises SEQ ID NO: 515 or SEQ ID NO: 520, or a sequence having at least 80%, at least 85%, at least 90%, at least 95%, at least 96%, at least 97%, at least 98%, or at least 99% or higher identity to SEQ ID NO: 515 or SEQ ID NO: 520.

In some embodiments, the antibody specifically binding to B-cell comprises the following formula: [antibody specifically binding to B-cell, heavy chain variable region (VH)]-[Linker A (optional)]-[antibody specifically binding to B-cell, light chain variable region (VL)]. In some embodiments, the antibody specifically binding to B-cell is an antibody that specifically binds to human CD22. In some embodiments, the antibody specifically binding to B-cell comprises an anti-CD22 ScFv. In some embodiments, the anti-CD22 ScFV comprises a heavy chain variable (VH) domain and an antibody light chain variable (VL) domain, wherein the VH and VL domains comprise: (1) a complementarity determining region-1 (CDR1), a CDR2, and a CDR3 of a heavy chain variable region (VH) comprising the amino acid sequence of SEQ ID NO: 433 and a CDR1, CDR2, and a CDR3 of a light chain variable region (VL) comprising the amino acid sequence of SEQ ID NO: 434; (2) a complementarity determining region-1 (CDR1), a CDR2, and a CDR3 of a heavy chain variable region (VH) comprising the amino acid sequence of SEQ ID NO: 447 and a CDR1, CDR2, and a CDR3 of a light chain variable region (VL) comprising the amino acid sequence of SEQ ID NO: 448; (3) a complementarity determining region-1 (CDR1), a CDR2, and a CDR3 of a heavy chain variable region (VH) comprising the amino acid sequence of SEQ ID NO: 457 and a CDR1, CDR2, and a CDR3 of a light chain variable region (VL) comprising the amino acid sequence of SEQ ID NO: 458; (4) a complementarity determining region-1 (CDR1), a CDR2, and a CDR3 of a heavy chain variable region (VH) comprising the amino acid sequence of SEQ ID NO: 481 and a CDR1, CDR2, and a CDR3 of a light chain variable region (VL) comprising the amino acid sequence of SEQ ID NO: 482; (5) a complementarity determining region-1 (CDR1), a CDR2, and a CDR3 of a heavy chain variable region (VH) comprising the amino acid sequence of SEQ ID NO: 495 and a CDR1, CDR2, and a CDR3 of a light chain variable region (VL) comprising the amino acid sequence of SEQ ID NO: 496; (6) a complementarity determining region-1 (CDR1), a CDR2, and a CDR3 of a heavy chain variable region (VH) comprising the amino acid sequence of SEQ ID NO: 509 and a CDR1, CDR2, and a CDR3 of a light chain variable region (VL) comprising the amino acid sequence of SEQ ID NO: 510; (7) a complementarity determining region-1 (CDR1), a CDR2, and a CDR3 of a heavy chain variable region (VH) comprising the amino acid sequence of SEQ ID NO: 523 and a CDR1, CDR2, and a CDR3 of a light chain variable region (VL) comprising the amino acid sequence of SEQ ID NO: 524; (8) a complementarity determining region-1 (CDR1), a CDR2, and a CDR3 of a heavy chain variable region (VH) comprising the amino acid sequence of SEQ ID NO: 525 and a CDR1, CDR2, and a CDR3 of a light chain variable region (VL) comprising the amino acid sequence of SEQ ID NO: 526; (9) a complementarity determining region-1 (CDR1), a CDR2, and a CDR3 of a heavy chain variable region (VH) comprising the amino acid sequence of SEQ ID NO: 527 and a CDR1, CDR2, and a CDR3 of a light chain variable region (VL) comprising the amino acid sequence of SEQ ID NO: 528; (10) a complementarity determining region-1 (CDR1), a CDR2, and a CDR3 of a heavy chain variable region (VH) comprising the amino acid sequence of SEQ ID NO: 529 and a CDR1, CDR2, and a CDR3 of a light chain variable region (VL) comprising the amino acid sequence of SEQ ID NO: 530; or (11) a complementarity determining region-1 (CDR1), a CDR2, and a CDR3 of a heavy chain variable region (VH) comprising the amino acid sequence of SEQ ID NO: 531 and a CDR1, CDR2, and a CDR3 of a light chain variable region (VL) comprising the amino acid sequence of SEQ ID NO: 532.

In some embodiments, the VH domain of the anti-CD22 ScFV comprises a CDR-H1 sequence comprising the amino acid sequence of SYGMH (SEQ ID NO: 427), a CDR-H2 sequence comprising the amino acid sequence of IIYYDGSKKYYADSVKG (SEQ ID NO: 428), and a CDR-H3 sequence comprising the amino acid sequence of ELTGDAFDI (SEQ ID NO: 429); and the VL domain of the anti-CD22 ScFV comprises a CDR-L1 sequence comprising the amino acid sequence of RASQSIGSSLH (SEQ ID NO: 430), a CDR-L2 sequence comprising the amino acid sequence of YASQSFS (SEQ ID NO:431), and a CDR-L3 sequence comprising the amino acid sequence of HQSSTLPYT (SEQ ID NO: 432). In some embodiments, the anti-CD22 ScFV comprises a VH domain comprising SEQ ID NO: 523, and a VL domain comprising SEQ ID NO: 524.

In some embodiments, the VH domain and the VL domain is connected through Linker A, wherein the Linker A is selected from the group consisting of SEQ ID Nos 337-348. In some embodiments, the Linker A is (GGGGS)4 (SEQ ID NO: 344).

In some embodiments, the Linker B is AS or AAA. In some embodiments, the hinge and transmembrane domains are derived from CD8 hinge and transmembrane domains.

In some embodiments, the hinge and transmembrane domains comprise SEQ ID NO: 538, SEQ ID NO: 539, or SEQ ID NO: 540. In some embodiments, the hinge and transmembrane domains are derived from CD28 hinge and transmembrane domains. In some embodiments, the CD28 hinge and transmembrane domains have the amino acid sequence selected from the group consisting of (i) SEQ ID NO: 522; (ii) sequences having at least 80%, at least 85%, at least 90%, at least 95%, at least 96%, at least 97%, at least 98%, or at least 99% or higher identity with SEQ ID NO: 522; and (iii) sequences that have 3, 2, or 1 amino acid difference with SEQ ID NO: 522.

In some embodiments, the Co-stimulatory domain is a CD28 Co-stimulatory domain. In some embodiments, the CD28 Co-stimulatory domain comprises SEQ ID NO: 543.

In some embodiments, the Signaling domain is derived from a CD3z signaling domain. In some embodiments, the Signaling domain has the amino acid sequence selected from the group consisting of (i) sequence of SEQ ID NO: 544; (ii) sequences that have at least 80% sequence identity with SEQ ID NO: 544; and (iii) sequences that have 3, 2, or 1 amino acid difference with SEQ ID NO: 544.

In some embodiments, the polypeptide encoded by the nucleic acid comprises or consists of SEQ ID NO: 116, SEQ ID NO: 126, or SEQ ID NO: 127. In some embodiments, the nucleic acid sequence encoding a polypeptide comprises a sequence encoding the polypeptide of SEQ ID NO: 116, SEQ ID NO: 126, or SEQ ID NO: 127.

In some embodiments, the nucleic acid sequence comprises SEQ ID NO: 108, SEQ ID NO: 124, or SEQ ID NO: 125. In some embodiments, the nucleic acid sequence comprises SEQ ID NO: 139 or SEQ ID NO: 147.

In some embodiments, the nucleic acid comprises pseudouridine. In some embodiments, the pseudouridine is N1-methyl-pseudouridine.

In some embodiments, the ionizable cationic lipid of the LNPs comprises a compound of Formula (I):

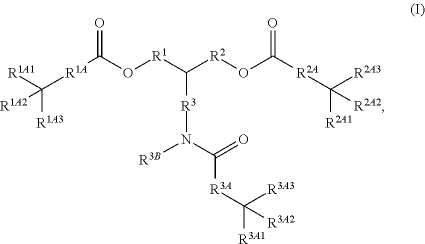

or a salt thereof, or both.

In some embodiments, R1, R2, and R³ are each independently a bond or C1-3 alkylene; R^{1A}, R2A, and R3A are each independently a bond or C1-10 alkylene; R1A1, R1A2, R1A3, R2A1, R2A2, R2A3, R3A1, R3A2, and R3A3 are each independently H, C1-20 alkyl, C1-20 alkenyl, —(CH2)0-10C(O)ORa1, or —(CH2)0-10OC(O)Ra2; Ra1 and R^{a2} are each independently C1-20 alkyl or C1-20 alkenyl; R3B is

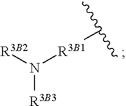

R3B1 is C1-6 alkylene; and R3B2 and R3B3 are each independently H, unsubstituted C1-6 alkyl, or C1-6 alkyl substituted with 1 or 2—OH. In some embodiments, R1, R2, and R3 are each independently a bond or methylene; R1A and R2A are each C1-10 alkylene; R3A is C1-5 alkylene; R1A1, R1A2, R2A1, R2A2, R^{3A1}, and R3A2 are each H; R1A3 and R2A3 are each C1-20 alkenyl; R3A3 is —C(O)O(C1-20 alkyl); R3B is

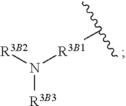

R3B1 is C2-4 alkylene; and R3B2 and R3B3 are each methyl. In some embodiments, R3B1 is —(CH2)3-. In some embodiments, the ionizable cationic lipid comprises

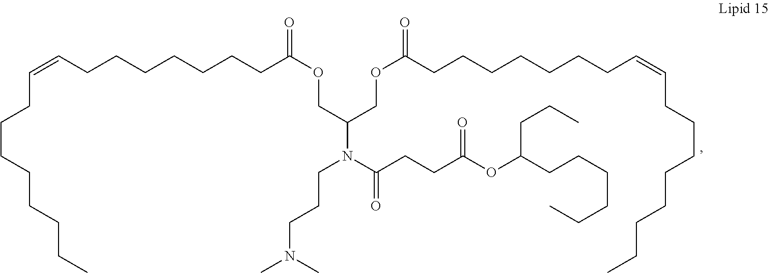 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof, or both.

In some embodiments, the cationic lipid has a concentration between about 10 mol % and about 60 mol % of the LNP. In some embodiments, the LNP comprises cationic lipid at a concentration between about 49 mol % and about 50 mol % of the LNP, such as about 49.1, 49.2, 49.3, 49.4, 49.5, 49.6, 49.7, 49.8, or 49.9 mol %.

In some embodiments, the LNP comprises cholesterol at a concentration between about 25 mol % and about 45 mol % of the LNP. In some embodiments, the LNP comprises cholesterol at a concentration between about 25 mol % and about 30 mol % of the LNP, such as about 25, 26, 27, 28, 29, or 30 mol %.

In some embodiments, the LNP comprises DSPC at a concentration between about 5 mol % and about 25% mol % of the LNP. In some embodiments, the LNP comprises DSPC at a concentration between about 9 mol % and about 21 mol % of the LNP.

In some embodiments, the LNP comprises DPG-PEG2K at a concentration between about 0.5 mol % and about 2.5 mol % of the LNP. In some embodiments, the LNP comprises DPG-PEG2K at a concentration between about 1.4 mol % and about 1.6 mol % of the LNP.

In some embodiments, the LNP comprises cationic lipid at a concentration between about 10 and about 20 g per gram of mRNA in the LNP. In some embodiments, the LNP comprises cholesterol at a concentration between about 3.0 and about 5.0 g per gram of mRNA in the LNP. In some embodiments, the LNP comprises DSPC at a concentration between about 2.0 and about 5.0 g per gram of mRNA in the LNP. In some embodiments, the LNP comprises DPG-PEG2K at a concentration between about 1.0 and about 1.5 g per gram of mRNA in the LNP.

In some embodiments, the LNP comprises DSPE-PEG3.4K-antibody conjugate at a concentration between about 0.05 to 0.1 g per gram of mRNA in the LNP. In some embodiments, the cationic lipid has a concentration about 49.2 mol % of the LNP; the cholesterol has a concentration about 39.4 mol % of the LNP; the DSPC has a concentration about 9.8 mol % of the LNP; and the DPG-PEG2K has a concentration about 1.5 mol % of the LNP.

In some embodiments, the cationic lipid has a concentration about 49.2 mol % of the LNP; the cholesterol has a concentration about 29.3 mol % of the LNP; the DSPC has a concentration about 20.0 mol % of the LNP; and the DPG-PEG2K has a concentration about 1.5 mol % of the LNP.

In some embodiments, the cationic lipid has a concentration about 14.2 g/g mRNA in the LNP; the cholesterol has a concentration about 4.64 g/g mRNA in the LNP; the DSPC has a concentration about 2.37 g/g mRNA in the LNP; the DPG-PEG2K has a concentration about 1.15 g/g mRNA in the LNP; and the DSPE-PEG3.4K-anti-CD8 antibody conjugate has a concentration about 0.084 g/g to 0.15 g/g mRNA in the LNP.

The present disclosure also provides isolated polynucleotides that have the following formula, arranged from 5′ to 3′: 5′Cap (optional)-5′ UTR (optional)-nucleotides encoding a Lead peptide sequence (optional)-nucleotides encoding an antibody heavy chain variable region (VH)-nucleotides encoding a Linker A (optional)-nucleotides encoding an antibody light chain variable region (VL)-nucleotides encoding Linker B (optional)-nucleotides encoding a Hinge-nucleotides encoding a Transmembrane domain-nucleotides encoding Co-stimulatory domain-nucleotides encoding Signaling domain-3′ UTR (optional)-polyA tail (optional), wherein the VH and VL form a binding domain that specifically binds to human B-cell. In some embodiments, the VH and VL forms a binding domain that specifically binds to human CD22. In some embodiments, the VH, the Linker A, and VL form an anti-CD22 ScFv. In some embodiments, the VH and VL domain comprises: (1) a complementarity determining region-1 (CDR1), a CDR2, and a CDR3 of a heavy chain variable region (VH) comprising the amino acid sequence of SEQ ID NO: 433 and a CDR1, CDR2, and a CDR3 of a light chain variable region (VL) comprising the amino acid sequence of SEQ ID NO: 434; (2) a complementarity determining region-1 (CDR1), a CDR2, and a CDR3 of a heavy chain variable region (VH) comprising the amino acid sequence of SEQ ID NO: 447 and a CDR1, CDR2, and a CDR3 of a light chain variable region (VL) comprising the amino acid sequence of SEQ ID NO: 448; (3) complementarity determining region-1 (CDR1), a CDR2, and a CDR3 of a heavy chain variable region (VH) comprising the amino acid sequence of SEQ ID NO: 457 and a CDR1, CDR2, and a CDR3 of a light chain variable region (VL) comprising the amino acid sequence of SEQ ID NO: 458; (4) a complementarity determining region-1 (CDR1), a CDR2, and a CDR3 of a heavy chain variable region (VH) comprising the amino acid sequence of SEQ ID NO: 481 and a CDR1, CDR2, and a CDR3 of a light chain variable region (VL) comprising the amino acid sequence of SEQ ID NO: 482; (5) a complementarity determining region-1 (CDR1), a CDR2, and a CDR3 of a heavy chain variable region (VH) comprising the amino acid sequence of SEQ ID NO: 495 and a CDR1, CDR2, and a CDR3 of a light chain variable region (VL) comprising the amino acid sequence of SEQ ID NO: 496; (6) a complementarity determining region-1 (CDR1), a CDR2, and a CDR3 of a heavy chain variable region (VH) comprising the amino acid sequence of SEQ ID NO: 509 and a CDR1, CDR2, and a CDR3 of a light chain variable region (VL) comprising the amino acid sequence of SEQ ID NO: 510; (7) a complementarity determining region-1 (CDR1), a CDR2, and a CDR3 of a heavy chain variable region (VH) comprising the amino acid sequence of SEQ ID NO: 523 and a CDR1, CDR2, and a CDR3 of a light chain variable region (VL) comprising the amino acid sequence of SEQ ID NO: 524; (8) a complementarity determining region-1 (CDR1), a CDR2, and a CDR3 of a heavy chain variable region (VH) comprising the amino acid sequence of SEQ ID NO: 525 and a CDR1, CDR2, and a CDR3 of a light chain variable region (VL) comprising the amino acid sequence of SEQ ID NO: 526; (9) a complementarity determining region-1 (CDR1), a CDR2, and a CDR3 of a heavy chain variable region (VH) comprising the amino acid sequence of SEQ ID NO: 527 and a CDR1, CDR2, and a CDR3 of a light chain variable region (VL) comprising the amino acid sequence of SEQ ID NO: 528; (10) a complementarity determining region-1 (CDR1), a CDR2, and a CDR3 of a heavy chain variable region (VH) comprising the amino acid sequence of SEQ ID NO: 529 and a CDR1, CDR2, and a CDR3 of a light chain variable region (VL) comprising the amino acid sequence of SEQ ID NO: 530; or (11) a complementarity determining region-1 (CDR1), a CDR2, and a CDR3 of a heavy chain variable region (VH) comprising the amino acid sequence of SEQ ID NO: 531 and a CDR1, CDR2, and a CDR3 of a light chain variable region (VL) comprising the amino acid sequence of SEQ ID NO: 532. In some embodiments, the VH domain of the anti-CD22 ScFV comprises a CDR-H1 sequence comprising the amino acid sequence of SYGMH (SEQ ID NO: 427), a CDR-H2 sequence comprising the amino acid sequence of IIYYDGSKKYYADSVKG (SEQ ID NO: 428), and a CDR-H3 sequence comprising the amino acid sequence of ELTGDAFDI (SEQ ID NO: 429); and the VL domain of the anti-CD22 ScFV comprises a CDR-L1 sequence comprising the amino acid sequence of RASQSIGSSLH (SEQ ID NO: 430), a CDR-L2 sequence comprising the amino acid sequence of YASQSFS (SEQ ID NO:431), and a CDR-L3 sequence comprising the amino acid sequence of HQSSTLPYT (SEQ ID NO: 432). In some embodiments, the anti-CD22 ScFV comprises a VH domain comprising SEQ ID NO: 523, and a VL domain comprising SEQ ID NO: 524. In some embodiments, the VH domain and the VL domain is connected through a Linker A, wherein the Linker A is selected from the group consisting of SEQ ID Nos 337-348. In some embodiments, the Linker A is (GGGGS) 4 (SEQ ID NO: 344). In some embodiments, the Linker B is AS or AAA. In some embodiments, the hinge and transmembrane domains are derived from CD8 hinge and transmembrane domains. In some embodiments, the hinge and transmembrane domains have at least 80%, at least 85%, at least 90%, at least 95%, at least 96%, at least 97%, at least 98%, at least 98%, at least 99%, or 100% identity with SEQ ID NO: 538, SEQ ID NO: 539, or SEQ ID NO: 540. In some embodiments, the hinge and transmembrane domains comprise SEQ ID NO: 538, SEQ ID NO: 539, or SEQ ID NO: 540. In some embodiments, the hinge and transmembrane domains are derived from CD28 hinge and transmembrane domains. In some embodiments, the CD28 hinge and transmembrane domains have the amino acid sequence selected from the group consisting of (i) sequence of SEQ ID NO. 522; (ii) sequences that have at least 80% sequence identity with SEQ ID NO: 522; and (iii) sequences that have 3, 2, or 1 amino acid difference with SEQ ID NO: 522. In some embodiments, the Co-stimulatory domain is a CD28 Co-stimulatory domain. In some embodiments, the CD28 Co-stimulatory domain has the amino acid sequence selected from the group consisting of (i) sequence of SEQ ID NO: 544; (ii) sequences that have at least 80% sequence identity with SEQ ID NO: 544; and (iii) sequences that have 3, 2, or 1 amino acid difference with SEQ ID NO: 544. In some embodiments, the Signaling domain is derived from a CD3z signaling domain. In some embodiments, the Signaling domain comprises or consists of SEQ ID NO: 544. In some embodiments, the polypeptide encoded by the polynucleotide comprises or consists of SEQ ID NO: 116, SEQ ID NO: 126, or SEQ ID NO: 127. In some embodiments, the polynucleotide encoding a polypeptide comprises a sequence encoding the polypeptide of SEQ ID NO: 116, SEQ ID NO: 126, or SEQ ID NO: 127. In some embodiments, the polynucleotide comprises SEQ ID NO: 108, SEQ ID NO: 124, or SEQ ID NO: 125, or the corresponding DNA sequence. In some embodiments, the nucleic acid sequence comprises SEQ ID NO: 139 or SEQ ID NO: 147, or the corresponding DNA sequence. In some embodiments, the nucleic acid comprises pseudouridine. In some embodiments, the pseudouridine is N1-methyl-pseudouridine.

The present disclosure also provides expression constructs comprising a polynucleotide described here.

The present disclosure also provides vectors comprising the expression construction described herein.

The present disclosure also provides host cells comprising the expression construct described herein.

The present disclosure also provides in vitro transcribed mRNA derived from the isolated polynucleotide described herein.

The present disclosure also provides immune cells comprising the in vitro transcribed mRNA described herein.

The present disclosure also provides recombinant polypeptides encoded by the isolated polynucleotide described herein.

The present disclosure also provides immune cells expressing the recombinant polypeptide described herein.

The present disclosure further provides methods of producing a polypeptide of interest in a cell, tissue, or bodily fluid of a subject. In some embodiments, the method comprises using the isolated polynucleotide as described herein.

The present disclosure further provides methods of preparing LNPs. In some embodiments, the method comprises combining the isolated polynucleotide described herein with mixture of lipids.

Also provided in the present disclosure, are pharmaceutical compositions. In some embodiments, the pharmaceutical compositions comprise LNPs as described herein. In some embodiments, the pharmaceutical compositions comprise the isolated polynucleotide as described herein. In some embodiments, the pharmaceutical compositions comprise the expression construct as described herein. In some embodiments, the pharmaceutical compositions comprise the vector as described herein. In some embodiments, the pharmaceutical compositions comprise the host cell and/or the recombinant polypeptide as described herein.

Also provided in the present disclosure, are methods of delivering a nucleic acid sequence into a human cell. In some embodiments, the methods comprise using the LNP of the present disclosure. In some embodiments, the LNP comprises the nucleic acid sequence to be delivered.

Also provided in the present disclosure, are methods of modulating immune response in a human subject. In some embodiments, the method comprises administering the LNP of the present disclosure, the isolated polynucleotide of the present disclosure, the expression construct of the present disclosure, the vector of the present disclosure, and/or the host cell of the present disclosure to the human subject, and/or expressing the recombinant polypeptide of the present disclosure in the human subject.

The present disclosure further provides methods of treating B-cell malignancy in a human subject in need thereof. In some embodiments, the methods comprise administering the LNP of the present disclosure, the isolated polynucleotide of the present disclosure, the expression construct of the present disclosure, the vector of the present disclosure, and/or the host cell of the present disclosure to the human subject, and/or expressing the recombinant polypeptide of the present disclosure in the human subject. In some embodiments, the B-cell malignancy is a B-cell lymphoma. In some embodiments, the B-cell lymphoma is diffuse large B-cell lymphoma (DLBCL).

The present disclosure further provides methods use of the LNPs, the isolated polynucleotides, the expression constructs, the vectors, the host cells and/or the pharmaceutical composition as described herein for the manufacture of a medicament for the treatment of a B-cell malignancy.

The present disclosure further provides methods use of the LNP of the present disclosure for the manufacture of a medicament for delivering a nucleic acid to a target cell. In some embodiments, the target cell is an immune cell.

The present disclosure further provides immunoglobulin single variable domains (ISVDs). In some embodiments, the ISVDs specifically bind human CD8alpha. In some embodiments, the ISVDs essentially consist of 4 framework regions (FR1 to FR4, respectively) and 3 complementarity determining regions (CDR1 to CDR3, respectively).

In some embodiments, in the ISVDs of the present disclosure, (i) CDR1 (according to AbM definition) has an amino acid sequence selected from the group consisting of: a) the amino acid sequence of SEQ ID NO: 244; b) amino acid sequences that have at least 80% amino acid identity with the amino acid sequence of SEQ ID NO: 244; and c) amino acid sequences that have 3, 2, or 1 amino acid difference with the amino acid sequences of SEQ ID NO: 244; and (ii) CDR2 (according to AbM definition) has an amino acid sequence selected from the group consisting of: d) the amino acid sequence of SEQ ID NO: 246, e) amino acid sequences that have at least 80% amino acid identity with the amino acid sequence of SEQ ID NO: 246; and f) amino acid sequences that have 3, 2, or 1 amino acid difference with the amino acid sequence of SEQ ID NO: 246; and (iii) CDR3 (according to AbM definition) has an amino acid sequence selected from the group consisting of: g) the amino acid sequence of SEQ ID NO: 248; h) amino acid sequences that have at least 80% amino acid identity with the amino acid sequence of SEQ ID NO: 248; and amino acid sequences that have 3, 2, or 1 amino acid difference with the amino acid sequence of SEQ ID NO: 248.

In some embodiments, in the ISVDs, the amino acid sequences of the CDRs (according to AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from the group consisting of SEQ ID NOs: 169 and SEQ ID NOs 160 to 168, SEQ ID NOs: 28-36 and 44, and SEQ ID NOs: 10 to 27.

In some embodiments, in the ISVDs described herein, CDR1 consists of the amino acid sequence of SEQ ID NO: 244; CDR2 consists of the amino acid sequence of SEQ ID NO: 246; and CDR3 consists of the amino acid sequence of SEQ ID NO: 248.

In some embodiments, the immunoglobulin single variable domain (ISVD) specifically binding human CD8alpha, that essentially consists of 4 framework regions (FR1 to FR4, respectively) and 3 complementarity determining regions (CDR1 to CDR3, respectively), in which: (i) CDR1 (according to Kabat definition) has an amino acid sequence selected from the group consisting of: a) the amino acid sequence of SEQ ID NO: 314; b) amino acid sequences that have at least 80% amino acid identity with the amino acid sequence of SEQ ID NO: 314; and c) amino acid sequences that have 3, 2, or 1 amino acid difference with the amino acid sequences of SEQ ID NO: 314; and (ii) CDR2 (according to Kabat definition) has an amino acid sequence selected from the group consisting of: d) the amino acid sequence of SEQ ID NO: 316; e) amino acid sequences that have at least 80% amino acid identity with the amino acid sequence of SEQ ID NO: 316; and f) amino acid sequences that have 3, 2, or 1 amino acid difference with the amino acid sequence of SEQ ID NO: 316; and (iii) CDR3 (according to Kabat definition) has an amino acid sequence selected from the group consisting of: g) the amino acid sequence of SEQ ID NO: 318; h) amino acid sequences that have at least 80% amino acid identity with the amino acid sequence of SEQ ID NO: 318; and amino acid sequences that have 3, 2, or 1 amino acid difference with the amino acid sequence of SEQ ID NO: 318. In some embodiments, the amino acid sequences of the CDRs (according to Kabat definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from the group consisting of SEQ ID NO: 179 and SEQ ID NOs 170 to 178.

In some embodiments, in the ISVDs described herein, CDR1 consists of the amino acid sequence of SEQ ID NO: 314; CDR2 consists of the amino acid sequence of SEQ ID NO: 316; and CDR3 consists of the amino acid sequence of SEQ ID NO: 318.

In some embodiments, in the ISVDs described herein, amino acid sequence of the ISVDs has 80% amino acid sequence identity with one of the amino acid sequences of SEQ ID NO: 169, SEQ ID Nos: 160 to 168, and SEQ ID Nos: 28-36 and 44, or any one of SEQ ID Nos: 10 to 27, in which for the purposes of determining the degree of amino acid identity, the amino acid residues that form the CDR sequences are disregarded; and preferably one or more of the amino acid residues at positions 11, 37, 44, 45, 47, 83, 84, 103, 104 and 108 according to the Kabat numbering are chosen from the Hallmark residues mentioned in Table A.

In some embodiments, the ISVDs described herein essentially consist of a heavy chain variable domain sequence that is derived from a conventional four-chain antibody or that essentially consist of a heavy chain variable domain sequence that is derived from heavy chain antibody. In some embodiments, the ISVDs essentially consist of a VHH, a humanized VHH, a camelized VH, a domain antibody, a single domain antibody, or a dAb, or any combination thereof. In some embodiments, the ISVD is a humanized ISVD. In some embodiments, the human ISVD is chosen from the group consisting of SEQ ID NO: 9, SEQ ID NO: 169 (EVOLVESGGGVVQPGGSLRLSCAASGFTFEDYAIGWFRQAPGKEREEVSCIRTYDEQTY YADSVKGRFTISRDNAKNTVSLQMNSLRPEDTALYYCAAGSYYACAYYSRPDPSEGHV DLDYWGQGTLVTVSS) and SEQ ID NOs 160 to 168, and SEQ ID NOs 28-36 and 44, or any one of SEQ ID Nos: 10 to 27, or from the group consisting of amino acid sequences that have more than 80%, preferably more than 90%, more preferably more than 95%, such as 99% or more amino acid sequence identity with at least one of the amino acid sequences of SEQ ID NO: 9, SEQ ID NO: 169 and SEQ ID NO 160 to 168, and SEQ ID NOs 28-36 and 44, or any one of SEQ ID Nos: 10 to 27.

In some embodiments, the ISVDs described herein comprise the amino acid sequence chosen from the group consisting of SEQ ID NO: 9, SEQ ID NO: 169 and SEQ ID NOs: 160 to 168, and SEQ ID NOs: 28-36 and 44, or any one of SEQ ID Nos: 10 to 27.

In some embodiments, the ISVDs described herein specifically bind to human CD8a with a dissociation constant (KD) of 5.10⁻⁹ to 10⁻¹¹ moles/litre or less, and preferably 10⁻⁹ to 5.10⁻¹¹ moles/litre or less and more preferably 5.10⁻¹⁰ to 10⁻¹⁰ moles/litre, as determined by Surface Plasmon Resonance.

In some embodiments, the ISVDs described herein specifically bind to human CD8α with a kon-rate of between 10⁵ M⁻¹s⁻¹ to about 10⁷ M⁻¹s⁻¹, preferably between 5.10⁵ M⁻¹s⁻¹ and 10⁷ M⁻¹s⁻¹, more preferably between 10⁶ M⁻¹s⁻¹ and 10⁷ M⁻¹s⁻¹, such as between 10⁶ M⁻¹s⁻¹ and 5.10⁶ M⁻¹s⁻¹, as determined by Surface Plasmon Resonance.

In some embodiments, the ISVDs described herein specifically bind to humanCD8α with a koff rate between 10⁻³ s⁻¹ (t½=0.69 s) and 10⁻⁶ s⁻¹ (providing a near irreversible complex with a t½ of multiple days), preferably between 10⁻³ s⁻¹ and 5.10⁻⁶ s⁻¹, more preferably between 5.10⁻⁴ s⁻¹ and 5.10⁻⁶ s⁻¹, such as between 5.10⁻⁴ s⁻¹ and 10⁻⁵ s⁻¹, as determined by Surface Plasmon Resonance.

In some embodiments, the ISVDs described herein specifically bind to human and cyno CD8α and does not bind to other T-cell surface glycoproteins.

In some embodiments, the ISVDs described herein antagonize an activity of CD8α, CD8α homodimer, and/or CD8α/CD8β heterodimer.

In some embodiments, the ISVDs described herein block the interaction of human CD8 co-receptor with human Major Histocompatibility Complex (MHC) class I protein with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁹ M, between 10⁻¹⁰ M and 10⁻⁸ M or between 10⁻¹¹ M and 10⁻⁹ M, for example, as measured in a FACS binding assay.

In some embodiments, the ISVDs described herein block the interaction of cyno CD8 co-receptor with cyno Major Histocompatibility Complex (MHC) class I protein with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁹ M, between 10⁻¹⁰ M and 10⁻⁸ M or between 10⁻¹¹ M and 10⁻⁹ M, for example, as measured in a FACS binding assay.

In some embodiments, the ISVDs described herein block the binding of hCD8 co-receptor to hMHC class I protein by at least 50%, such as at least 60%, 70%, 80%, 90%, 95%, 99% or even more, as determined by ligand competition, AlphaScreen, or competitive binding assays (such as competition ELISA or competition FACS).

In some embodiments, the ISVDs described herein block the interaction of CD8 co-receptor with lymphocyte-specific protein tyrosine kinase with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁸ M, between 10⁻¹⁰ M and 10⁻⁹ M or between 10⁻¹¹ M and 10⁻⁹ M, as determined in a functional assay.

The present disclosure further provides polypeptides or constructs that comprises or essentially consists of one or more ISVDs described herein, or nucleic sequences encoding the ISVDs. In some embodiments, the polypeptides or constructs optionally further comprise one or more other groups, residues, moieties or binding units, optionally linked via one or more linkers. In some embodiments, said one or more other groups, residues, moieties or binding units are amino acid sequences. In some embodiments, said one or more linkers are one or more amino acid sequences. In some embodiments, said one or more other groups, residues, moieties or binding units are immunoglobulin sequences. In some embodiments, said one or more other groups, residues, moieties or binding units are ISVDs. In some embodiments, said one or more other groups, residues, moieties or binding units are chosen from the group consisting of VHHs, humanized VHHs, camelized VHs, domain antibodies, single domain antibodies and dAbs. In some embodiments, the construct as described herein is a multivalent construct. In some embodiments, the is a multispecific construct. In some embodiments, said one or more other groups, residues, moieties or binding units provide the polypeptide or construct with increased half-life, compared to the ISVD without the one or more other groups, residues, moieties or binding units.

In some embodiments, said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.

In some embodiments, said one or more other groups, residues, moieties or binding units that provide the polypeptide or construct with increased half-life is chosen from the group consisting of human serum albumin or fragments thereof.

In some embodiments, said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).

In some embodiments, said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).

In some embodiments, said one or more other groups, residues, moieties or binding units that provides the polypeptide or construct with increased half-life is an ISVD that specifically binds human serum albumin. In some embodiments, said ISVD that specifically binds human serum albumin essentially consists of 4 framework regions (FR1 to FR4, respectively) and 3 complementarity determining regions (CDR1 to CDR3 respectively), in which: (i) CDR1 (according to AbM definition) has an amino acid sequence selected from the group consisting of: a) the amino acid sequence of GFTFRSFGMS (SEQ ID NO:566); b) amino acid sequences that have at least 80% amino acid identity with the amino acid sequence of SEQ ID NO: 566; and c) amino acid sequences that have 3, 2, or 1 amino acid difference with the amino acid sequences of SEQ ID NO:566; and (ii) CDR2 (according to AbM definition) has an amino acid sequence selected from the group consisting of: d) the amino acid sequence of SISGSGSDTL (SEQ ID NO: 567); e) amino acid sequences that have at least 80% amino acid identity with the amino acid sequence of SEQ ID NO:567; f) amino acid sequences that have 3, 2, or 1 amino acid difference with the amino acid sequence of SEQ ID NO:567; and (iii) CDR3 (according to AbM definition) has an amino acid sequence selected from the group consisting of: g) the amino acid sequence of GGSLSR (SEQ ID NOs: 568); h) amino acid sequences that have at least 80% amino acid identity with the amino acid sequence of SEQ ID NO:568; i) amino acid sequences that have 3, 2, or 1 amino acid difference with the amino acid sequence of SEQ ID NO:568. In some embodiments, in the ISVDs of the present disclosure, CDR1 consists of the amino acid sequence of SEQ ID NO:566, CDR2 consists of the amino acid sequence of SEQ ID NO:567, and CDR3 consists of the amino acid sequence of SEQ ID NO:568.

In some embodiments, said ISVD that specifically binds human serum albumin is selected from the group consisting of ALB8 (SEQ ID NO: 320), ALB23 (SEQ ID NO: 321), ALBX00001 (SEQ ID NO: 334) and ALB23002 (SEQ ID NO: 335).

In some embodiments, the polypeptides or constructs optionally further comprise one or more other groups, residues, moieties or binding units, optionally linked via one or more linkers. In some embodiments, said linker is chosen from the group consisting of SEQ ID NOs: 336 to 352.

In some embodiments, the polypeptide or construct of the present disclosure further comprises a C-terminal extension. In some embodiments, said C-terminal extension is a C-terminal extension (X)n, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

The present disclosure further provides nucleic acids that encode an ISVD or a polypeptide as described herein. In some embodiments, the nucleic acid is in a genetic construct.

The present disclosure further provides non-human host or host cells. In some embodiments, non-human host or host cells comprise the nucleic acid as described herein. In some embodiments, the non-human host or host cells expresses, or that under suitable circumstances is capable of expressing, an ISVD or a polypeptide as described herein.

The present disclosure further provides methods for producing an ISVD or a polypeptide. In some embodiments, the methods comprise a) expressing, in a suitable non-human host cell or host organism or in another suitable expression system, a nucleic acid described herein. In some embodiments, the methods further optionally comprise b) isolating and/or purifying the ISVD or the polypeptide.

The present disclosure further provides methods for producing an ISVD or a polypeptide. In some embodiments, the methods comprise a) cultivating and/or maintaining a non-human host or host cell under conditions that are such that said non-human host or host cell expresses and/or produces at least one ISVD as described herein, or at least one polypeptide as described herein. In some embodiments, the methods further optionally comprise b) isolating and/or purifying the ISVD or the polypeptide.

The present disclosure further provides compositions comprising at least one ISVD, at least one polypeptide or construct, or at least one nucleic acid as described herein, or any combination thereof. In some embodiments, the composition is a pharmaceutical composition. In some embodiments, the pharmaceutical composition further comprises at least one pharmaceutically acceptable carrier, diluent or excipient and/or adjuvant, and that optionally comprises one or more further pharmaceutically active polypeptides and/or compounds.

The present disclosure further provides the ISVD, the polypeptide, or the construct, or the composition as described herein, for use as a medicament.

The present disclosure further provides the ISVD, the polypeptide, or the construct, or the composition as described herein, for use in the diagnosis, prevention and/or treatment of at least one disease and/or disorder.

The present disclosure further provides the ISVD, the polypeptide, or the construct, or the composition as described herein, for use in the diagnosis, prevention and/or treatment of at least one disease and/or disorder that is associated with CD8alpha, with its biological or pharmacological activity, and/or with the biological pathways or signaling in which CD8alpha is involved.

The present disclosure further provides the ISVD, the polypeptide, or the construct, or the composition as described herein, for use in the diagnosis, prevention and/or treatment of an immunological disease, an infectious disease, or a proliferative disease, such as B cell leukemias and lymphomas.

The present disclosure further provides methods for the diagnosis, prevention and/or treatment of at least one disease and/or disorder. In some embodiments, the methods comprise the administration, to a subject, of the ISVD, the polypeptide, the construct, the composition as described herein. In some embodiments, the disease or disorder that is associated with CD8alpha, with its biological or pharmacological activity, and/or with the biological pathways or signaling in which CD8alpha is involved. In some embodiments, said methods comprise administering, to a subject, at least one ISVD, a polypeptide, a construct, or a composition as described herein.

The present disclosure further provides immunoglobulin single variable domains (ISVDs) specifically binding human CD8alpha, that essentially consists of 4 framework regions (FR1 to FR4, respectively) and 3 complementarity determining regions (CDR1 to CDR3, respectively), in which: (i) CDR1 (according to AbM definition) has an amino acid sequence selected from the group consisting of: a) the amino acid sequence of SEQ ID NO. 181 or 188; b) amino acid sequences that have at least 80% amino acid identity with the amino acid sequence of SEQ ID NO: 181 or 188; and c) amino acid sequences that have 3, 2, or 1 amino acid difference with the amino acid sequences of SEQ ID NO: 181 or 188; and (ii) CDR2 (according to AbM definition) has an amino acid sequence selected from the group consisting of: d) the amino acid sequence of SEQ ID NO: 183 or 190; e) amino acid sequences that have at least 80% amino acid identity with the amino acid sequence of SEQ ID NO:183 or 190; f) amino acid sequences that have 3, 2, or 1 amino acid difference with the amino acid sequence of SEQ ID NO: 183 or 190; and CDR3 (according to AbM definition) has an amino acid sequence selected from the group consisting of: g) the amino acid sequence of SEQ ID NO: 185, 192, 199 or 206; h) amino acid sequences that have at least 80% amino acid identity with the amino acid sequence of SEQ ID NO: 185, 192, 199 or 206; or i) amino acid sequences that have 3, 2, or 1 amino acid difference with the amino acid sequence of SEQ ID NO: 185, 192, 199 or 206. In some embodiments, the ISVD is selected from the group consisting of: a) an ISVD comprising SEQ ID NOs: 181, 183, and 185; b) an ISVD comprising SEQ ID NOs: 188, 190, and 192; c) an ISVD comprising SEQ ID NOs: 188, 190, and 199; d) an ISVD comprising SEQ ID NOs: 188, 190, and 206. In some embodiments, the FR1 to FR4 in the ISVD is selected from the group consisting of: a) an FR1 comprising SEQ ID NO: 180 or 187; b) an FR2 comprising SEQ ID NO: 182; c) an FR3 comprising SEQ ID NO: 184, 191, 198 or 233; and d) an FR4 comprising SEQ ID NO: 186 or 193. In some embodiments, the ISVD is selected from the group consisting of: a) an ISVD comprising SEQ ID NO: 160; b) an ISVD comprising SEQ ID NO: 161; c) an ISVD comprising SEQ ID NO: 162; d) an ISVD comprising SEQ ID NO: 163; e) an ISVD comprising SEQ ID NO: 164; f) an ISVD comprising SEQ ID NO: 165; g) an ISVD comprising SEQ ID NO: 166; h) an ISVD comprising SEQ ID NO: 167; and i) an ISVD comprising SEQ ID NO: 168.

The present disclosure further provides immunoglobulin single variable domains (ISVDs) specifically binding human CD8alpha, that essentially consists of 4 framework regions (FR1 to FR4, respectively) and 3 complementarity determining regions (CDR1 to CDR3, respectively), in which: (i) CDR1 (according to Kabat definition) has an amino acid sequence selected from: a) the amino acid sequence of SEQ ID NO: 251; b) amino acid sequences that have at least 80% amino acid identity with the amino acid sequence of SEQ ID NO: 251; c) amino acid sequences that have 3, 2, or 1 amino acid difference with the amino acid sequences of SEQ ID NO: 251; and (ii) CDR2 (according to Kabat definition) has an amino acid sequence selected from: d) the amino acid sequence of SEQ ID NO: 253 or 260; e) amino acid sequences that have at least 80% amino acid identity with the amino acid sequence of SEQ ID NO: 253 or 260; f) amino acid sequences that have 3, 2, or 1 amino acid difference with the amino acid sequence of SEQ ID NO: 253 or 260; and (iii) CDR3 (according to Kabat definition) has an amino acid sequence selected from: g) the amino acid sequence of SEQ ID NO: 255, 262, 269, or 276; h) amino acid sequences that have at least 80% amino acid identity with the amino acid sequence of SEQ ID NO: 255, 262, 269, or 276; and i) amino acid sequences that have 3, 2, or 1 amino acid difference with the amino acid sequence of SEQ ID NO: 255, 262, 269, or 276. In some embodiments, the ISVD is selected from the group consisting of: a) an ISVD comprising SEQ ID NOs: 251, 253, and 255; b) an ISVD comprising SEQ ID NOs: 258, 260, and 262; c) an ISVD comprising SEQ ID NOs: 265, 267, and 269; and d) an ISVD comprising SEQ ID NOs: 272, 274, and 276. In some embodiments, the FR1 to FR4 in the ISVD is selected from the group consisting of: a) an FR1 comprising SEQ ID NO: 250 or 257; b) an FR2 comprising SEQ ID NO: 252; c) an FR3 comprising SEQ ID NO: 254, 261, 282, or 303; and d) an FR4 comprising SEQ ID NO: 256 or 263. In some embodiments, the ISVD is selected from the group consisting of: a) an ISVD comprising SEQ ID NO: 170; b) an ISVD comprising SEQ ID NO: 171; c) an ISVD comprising SEQ ID NO: 172; d) an ISVD comprising SEQ ID NO: 173; e) an ISVD comprising SEQ ID NO: 174; f) an ISVD comprising SEQ ID NO: 175; g) an ISVD comprising SEQ ID NO: 176; h) an ISVD comprising SEQ ID NO: 177; and i) an ISVD comprising SEQ ID NO: 178. In some embodiments, the ISVD has a cysteine containing linker at its C-terminal end. In some embodiments, the cysteine containing linker is a GGC linker. In some embodiments, the ISVD comprises a C-terminal extension sequence of any one of SEQ ID Nos: 353 to 371. In some embodiments, the C-terminal extension consists of VTVSS(X)n (SEQ ID NO: 353). In some embodiments, the C-terminal extension consists of VTVSS (SEQ ID NO: 371).

The present disclosure further provides conjugates comprising an ISVD as described herein linked to a phospholipid-PEG-maleimide derivative. In some embodiments, the phospholipid-PEG-maleimide derivative is a derivative of phosphatidylethanolamine. In some embodiments, the phospholipid-PEG-maleimide derivative comprises stearic acid acyl chains. In some embodiments, the phospholipid is 1,2-Distearoyl-sn-glycero-3-phosphoethanolamine-maleimide (DSPE). In some embodiments, the PEG has a molecular weight from about 1.5 kDa to about 6 kDa. In some embodiments, the PEG has a molecular weight of 2 kDa, 3.4 kDa or 5 kDa. In some embodiments, the PEG has a molecular weight of 3.4 kDa. In some embodiments, the phospholipid-PEG-maleimide derivative is DSPE-PEG 3.4 K-maleimide.

The present disclosure further provides methods for the preparation of a composition comprising monomers of an ISVD with a cysteine containing linker at its C-terminal end. In some embodiments, the methods comprise the following sequential steps: (a) reducing a composition comprising ISVD dimers to ISVD monomers with a first reducing agent, wherein the ISVD dimers are formed through the cysteine containing linker at the C-terminal end of the ISVD. In further embodiments, the methods comprise (b) purifying the ISVD monomers obtained in step (a) to get a purified composition comprising the ISVD monomers. In further embodiments, the methods comprise (c) reducing the purified composition obtained in step (b) with a second reducing agent. In some embodiments, the methods further comprise (d) purifying the reduced composition obtained in step (c) to obtain a composition comprising monomers of the ISVD. In some embodiments, the composition comprising the ISVD dimers in step (a) is obtained through expressing the ISVD in a host cell. In some embodiments, the composition comprises the ISVD dimers is purified to remove host cell proteins and DNA before being subjected to step (a). In some embodiments, the first reducing agent comprises tris(2-carboxyethyl)phosphine (TCEP). In some embodiments, the step (a) is conducted around 15 to 25° C., optionally around 20-22° C. In some embodiments, the step (a) takes about 16 to 20 hours. In some embodiments, the step (b) comprises using a chromatography. In some embodiments, the chromatography comprises an ion exchange chromatography (IEX). In some embodiments, the second reducing agent in step (c) comprises tris(2-carboxyethyl)phosphine (TCEP). In some embodiments, the step (c) is conducted around 15 to 25° C., optionally around 20-22° C. In some embodiments, the step (c) takes about 16 to 20 hours. In some embodiments, the step (d) comprises Ultrafiltration/Diafiltration (UF/DF). In some embodiments, at least 80% of the ISVD in the composition obtained in step (d) is in monomeric form. In some embodiments, the cysteine containing linker is a GGC linker. In some embodiments, the C-terminal end comprises the sequence VTVSS (SEQ ID NO: 371) before the cysteine linker. In some embodiments, the ISVD comprises two internal disulphide bridges. In some embodiments, before being subjected to step (a), the ISVD dimers is purified using protein A chromatography to remove host cell proteins and DNA. In some embodiments, both the first reducing agent and second reducing agent comprise TCEP. In some embodiments, the first reducing agent comprises 20×TCEP. In some embodiments, the second reducing agent comprise 10×TCEP. In some embodiments, the UF/DF membrane has a molecular weight cut-off of 10 kDa.

The present disclosure further provides methods for the preparation of a phospholipid-PEG-ISVD conjugate. In some embodiments, the methods comprise the following sequential steps: (a) mixing a first composition comprising monomers of an ISVD comprising a cysteine containing linker, with a second composition comprising phospholipid-PEG molecules comprising a bioconjugation linker under conditions that the phospholipid-PEG molecules and the ISVD monomers can form a conjugate through clicking chemistry; and (b) adding cysteine to the conjugate obtained in step (a) under conditions that the conjugation reaction is quenched, wherein a composition comprising the phospholipid-PEG ISVD conjugate is obtained. In some embodiments, at least 80% of the ISVD in the first composition is in monomeric form. In some embodiments, the phospholipid in the phospholipid-PEG is a derivative of phosphatidylethanolamine. In some embodiments, the phospholipid comprises stearic acid acyl chains. In some embodiments, the phospholipid is 1,2-Distearoyl-sn-glycero-3-phosphoethanolamine (DSPE). In some embodiments, the PEG has a molecular weight of about 1.5 kDa to about 6.5 kDa. In some embodiments, the PEG has a molecular weight of about 2 kDa, about 3.4 kDa, or about 5 kDa. In some embodiments, the PEG has a molecular weight of 3.4 kDa. In some embodiments, the conjugate is a DSPE-PEG 3.4K-ISVD conjugate. In some embodiments, the bioconjugation linker in the phospholipid-PEG has a maleimide group. In some embodiments, the second composition further comprises molecules of a second phospholipid-PEG that does not have the bioconjugation linker in addition to the phospholipid-PEG molecules comprising the bioconjugation linker. In some embodiments, the size of PEG in the second phospholipid-PEG is different compared to the size of PEG in the phospholipid-PEG comprising the bioconjugation linker. In some embodiments, the size of PEG in the second phospholipid-PEG is smaller compared to the size of PEG in the phospholipid-PEG comprising the bioconjugation linker. In some embodiments, the size of PEG in the second phospholipid-PEG is about 2 kDa, and the size of PEG in the phospholipid-PEG comprising the bioconjugation linker is 3.4 KDa. In some embodiments, the second composition comprises DSPE-PEG 3.4 kDa with a bioconjugation linker, and DSPE-PEG 2.0 kDa without the bioconjugation linker. In some embodiments, the DSPE-PEG 2.0 kDa has the structure below or a salt thereof:

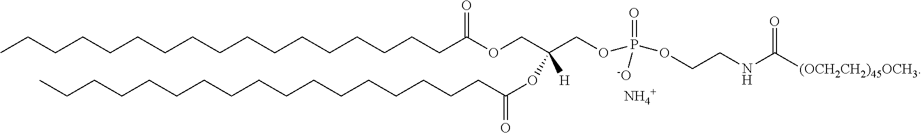

(DSPE-PEG2.0 kDa-OCH3). In some embodiments, the DSPE-PEG 3.4 kDa has a maleimide linker. In some embodiments, the cysteine containing linker in the ISVD is a GGC linker. In some embodiments, the cysteine containing linker comprising a sequence of any one of SEQ ID Nos: 353 to 370. In some embodiments, the ISVD comprising VTVSS(X)n (SEQ ID NO: 353) before the GGC linker. In some embodiments, the ISVD comprising VTVSS (SEQ ID NO: 371) before the GGC linker. In some embodiments, the molar ratio of the phospholipid-PEG molecules comprising the bioconjugation linker to the second phospholipid-PEG that does not have the bioconjugation linker is about 1:3 to about 1:1. In some embodiments, the molar ratio of the phospholipid-PEG molecules comprising the bioconjugation linker to the second phospholipid-PEG that does not have the bioconjugation linker is about 2:3. In some embodiments, the molar ratio of the ISVD monomers, the phospholipid-PEG molecules comprising the bioconjugation linker, and the second phospholipid-PEG that does not have the bioconjugation linker is about 1:1:4 or 1:2:3. In some embodiments, a clicking chemistry reaction takes place in the mixture in step (a) under 15 to 25° C., or optionally under 20 to 22° C. In some embodiments, a clicking chemistry reaction takes place in the mixture in step (a) for about 2 hours. In some embodiments, the molar ratio of the cysteine added in step (b) for quenching the conjugation reaction to the phospholipid-PEG molecules comprising a bioconjugation linker is at least 3. In some embodiments, the molar ratio is about 3.1 to about 4.1. In some embodiments, the quenching in step (b) is carried out for about 30 min. In some embodiments, the quenching in step (b) takes place under 15 to 25° C., or optionally under 20 to 22° C. In some embodiments, the method further comprises purifying the obtained composition comprising the phospholipid-PEG ISVD conjugate using ultrafiltration/diafiltration (UF/DF). In some embodiments, the UF/DF has a molecular weight cut-off of about 10 kDa. In some embodiments, the composition comprising the phospholipid-PEG ISVD conjugate is formulated in buffer. In some embodiments, the buffer comprises HEPES pH7.4, NaCl, and sucrose. In some embodiments, the buffer comprises 1.5 mM HEPES pH7.4, 150 mM NaCl, and 10% sucrose buffer.

The present disclosure further provides phospholipid-PEG-ISVD conjugates produced by the method as described herein.

The present disclosure further provides a composition comprising a phospholipid-PEG-ISVD conjugate produced by the method as described herein. In some embodiments, the composition comprises micelles comprising the phospholipid-PEG-ISVD conjugate. In some embodiments, the micelles comprise the phospholipid-PEG-ISVD conjugate, and a second phospholipid-PEG molecule that does not have a bioconjugation linker. In some embodiments, the micelles comprise DSPE-PEG 3.4K-anti-CD8α ISVD conjugate, and DSPE-PEG2.0k-OMeH. In some embodiments, the molar ratio among the CD8α ISVD, DSPE-PEG 3.4K, and DSPE-PEG2.0K is about 1:1:4 or 1:2:3. In some embodiments, the CD8α ISVD comprises or consists of SEQ ID NO: 44.

The present disclosure further provides methods of producing a composition comprising lipid nanoparticles (LNPs), wherein the LNPs comprising: (a) a lipid-immune cell targeting group conjugate comprising the compound of Formula (II): [Lipid]-[optional linker]-[antibody], (b) an ionizable cationic lipid, (c) a nucleic acid, wherein the nucleic acid is encapsulated in the LNP, (d) a structural lipid (e.g., a sterol), (e) a neutral phospholipid, and (f) a free PEG-lipid. In some embodiments, the method comprises: (i) producing a first composition comprising the lipid-immune cell targeting group conjugate in (a); (ii) producing a second composition comprising (b) to (f); and (iii) incubating the first composition obtained from step (i) and the second composition obtained from step (ii), to produce the final composition comprising the LNPs. In some embodiments, the antibody in the lipid-immune cell targeting group conjugate comprises an ISVD. In some embodiments, the lipid-immune cell targeting group conjugate is a phospholipid-PEG-ISVD. In some embodiments, the phospholipid-PEG-ISVD is produced by the method of a method as described herein. In some embodiments, the phospholipid-PEG-ISVD is DSPE-PEG3.4K-ISVD. In some embodiments, the ISVD is an anti-CD8 ISVD. In some embodiments, the anti-CD8 ISVD comprises a sequence selected from the group consisting of SEQ ID NOs: 160 to 169, and SEQ ID NOs: 28-36 and 44, and SEQ ID NOs: 10 to 27. In some embodiments, the anti-CD8 ISVD comprises SEQ ID NO: 44. In some embodiments, the LNPs comprises Lipid 15, DSPC, Cholesterol, DPG-PEG, DSPE-PEG3.4K-A044300805_v8_GGC (SEQ ID NO: 44), and mRNA encoding a CD22 CAR. In some embodiments, the mRNA comprises SEQ ID NO: 139 or SEQ ID NO: 147. In some embodiments, the mRNA is produced through in vitro transcription. In some embodiments, the mRNA comprises pseudouridine. In some embodiments, the pseudouridine is N1-methyl-pseudouridine.

The present disclosure further provides compositions produced by methods of producing a composition comprising lipid nanoparticles (LNPs) as described herein.

### DESCRIPTION OF THE FIGURES

The patent or application file contains at least one drawing executed in color. Copies of this patent or patent application publication with color drawings will be provided by the Office upon request and payment of the necessary fee.

The present application can be understood by reference to the following description taken in conjunction with the accompanying figures.

FIG. 1 depicts proton NMR spectrum of intermediate 13-11.

FIG. 2A depicts proton NMR spectrum of intermediate 13-11a; FIG. 2B depicts proton NMR spectrum of intermediate 13-11b; and FIG. 2C depicts LC-ELSD of intermediate 13-11b.

FIG. 3A depicts proton NMR spectrum of intermediate 13-10; FIG. 3B depicts LC-CAD chromatogram of intermediate 13-10.

FIG. 4A-1 depicts proton NMR spectrum for Lipid 1; FIG. 4A-2 depicts the LC-CAD chromatogram of Lipid 1.

FIG. 4B-1 depicts proton NMR spectrum of Lipid 3; FIG. 4B-2 depicts the LC-CAD chromatogram of Lipid 3.

FIG. 4C-1 depicts proton NMR spectrum of Lipid 4; FIG. 4C-2 depicts the LC-CAD chromatogram L of Lipid 4.

FIG. 4D-1 depicts proton NMR spectrum of Lipid 5A; FIG. 4D-2 depicts the LC-CAD chromatogram of Lipid 5A.

FIG. 4E-1 depicts proton NMR spectrum of Lipid 6; FIG. 4E-2 depicts the LC-CAD chromatogram of Lipid 6.

FIG. 4F-1 depicts proton NMR spectrum of Lipid 7; FIG. 4F-2 depicts the LC-CAD chromatogram of Lipid 7.

FIG. 4G-1 depicts proton NMR spectrum of Lipid 2; FIG. 4G-2 depicts the LC-CAD chromatogram of Lipid 2;

FIG. 4H-1 depicts proton NMR spectrum of Lipid 8; FIG. 4H-2 depicts the LC-CAD chromatogram of Lipid 8.

FIG. 4I-1 depicts proton NMR spectrum of Lipid 9; FIG. 4I-2 depicts the LC-CAD chromatogram of Lipid 9.

FIG. 4J-1 depicts proton NMR spectrum of Lipid 10A; FIG. 4J-2 depicts the LC-CAD chromatogram of Lipid 10A.

FIG. 4K-1 depicts proton NMR spectrum of Lipid 11A; FIG. 4K-2 depicts the LC-CAD chromatogram of Lipid 11A.

FIG. 4L-1 depicts proton NMR spectrum of Lipid 12; FIG. 4L-2 depicts the LC-CAD chromatogram of Lipid 12.

FIG. 4M-1 depicts proton NMR spectrum of Lipid 13; FIG. 4M-2 depicts the LC-CAD chromatogram of Lipid 13.

FIG. 4N-1 depicts proton NMR spectrum of Lipid 15; FIG. 4N-2 depicts the LC-CAD chromatogram of Lipid 15.

FIG. 4O-1 depicts proton NMR spectrum of Lipid 16; FIG. 4O-2 depicts the LC-CAD of Lipid 16.

FIG. 4P-1 depicts proton NMR spectrum of Lipid 19; FIG. 4P-2 depicts the LC-ELSD chromatogram of Lipid 19.

FIG. 4Q-1 depicts proton NMR spectrum of Lipid 20; FIG. 4Q-2 depicts the LC-ELSD chromatogram of Lipid 20.

FIG. 4R-1 depicts proton NMR spectrum of Lipid 31; FIG. 4R-2 depicts the LC-CAD chromatogram of Lipid 31.

FIG. 4S-1 depicts proton NMR spectrum of Lipid 32; FIG. 4S-2 depicts the LC-CAD chromatogram of Lipid 32.

FIG. 4T-1 depicts proton NMR spectrum of Lipid 33; FIG. 4T-2 depicts the LC-CAD chromatogram of Lipid 33.

FIG. 4U-1 depicts proton NMR spectrum of Lipid 34; FIG. 4U-2 depicts the LC-CAD chromatogram of Lipid 34.

FIG. 4V-1 depicts proton NMR spectrum of Lipid 14A; FIG. 4V-2 depicts the LC-CAD chromatogram of Lipid 14A.

FIG. 4W-1 depicts proton NMR spectrum of Lipid 17A; FIG. 4W-2 depicts the LC-CAD chromatogram of Lipid 17A.

FIG. 4X-1 depicts proton NMR spectrum of Lipid 18A; FIG. 4X-2 depicts the LC-CAD chromatogram of Lipid 18A.

FIG. 4Y-1 depicts proton NMR spectrum of Lipid 21A; FIG. 4Y-2 depicts the LC-CAD chromatogram of Lipid 21A.

FIG. 4Z-1 depicts proton NMR spectrum of Lipid 22; FIG. 4Z-2 depicts the LC-CAD chromatogram of Lipid 22.

FIG. 4AA-1 depicts proton NMR spectrum of Lipid 23A; FIG. 4AA-2 depicts the LC-CAD chromatogram of Lipid 23A.

FIG. 4AC-1 depicts proton NMR spectrum of Lipid 25A; FIG. 4AC-2 depicts the LC-CAD chromatogram of Lipid 25A.

FIG. 4AE-1 depicts proton NMR spectrum of Lipid 27; FIG. 4AE-2 depicts the LC-CAD chromatogram of Lipid 27.

FIG. 4AF-1 depicts proton NMR spectrum of Lipid 28; FIG. 4AF-2 depicts the LC-CAD chromatogram of Lipid 28.

FIG. 4AG-1 depicts proton NMR spectrum of Lipid 29; FIG. 4AG-2 depicts the LC-CAD chromatogram of Lipid 29.

FIG. 4AH-1 depicts proton NMR spectrum of Lipid 37A; FIG. 4AH-2 depicts the LC-CAD chromatogram of Lipid 37A.

FIG. 4AI-1 depicts proton NMR spectrum of Lipid 19A; FIG. 4AI-2 depicts the LC-CAD chromatogram of Lipid 19A.

FIG. 4AJ-1 depicts proton NMR spectrum of Lipid 20A; FIG. 4AJ-2 depicts the LC-CAD chromatogram of Lipid 20A.

FIG. 5A depicts diameter (DLS, nm) of LNPs based on Lipid 1 to Lipid 8 in pH 7.4 HBS, pH 6.5 MBS, Post antibody (αCD3, hSP34) insertion and post freeze-thaw (−80° C.).

FIG. 5B depicts polydispersity (DLS) of LNPs based on Lipid 1 to Lipid 8 in pH 7.4 HBS, pH 6.5 MBS, Post antibody (αCD3, hSP34) insertion and post freeze-thaw (−80° C.).

FIG. 5C depicts charge (Zeta potential, DLS) of LNPs based on Lipid 1 to Lipid 8 in pH 5.5 MBS, pH 7.4 HBS.

FIG. 5D depicts % RNA recovery and dye accessible RNA in LNPs based on Lipid 1 to Lipid 8.

FIG. 6A depicts diameter (DLS, nm) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6B depicts polydispersity (DLS) of LNPs based on Lipids 9, 10, 11, and 15 in pH 7.4 HBS, pH 6.5 MBS, Post antibody (αCD3, hSP34) insertion and post freeze-thaw (−80° C.).

FIG. 6C depicts charge (Zeta potential, DLS) of LNPs based on Lipids 9, 10, 11, and 15 in pH 5.5 MBS, pH7.4 HBS.

FIG. 6D depicts % RNA recovery and dye accessible RNA in LNPs based on Lipids 9, 10, 11, and 15.

FIG. 7A depicts diameter (DLS, nm) of LNPs based on Lipid 31 to Lipid 34 in pH 7.4 HBS, pH 6.5 MBS, Post antibody (αCD3, hSP34) insertion and post freeze-thaw (−80° C.).

FIG. 7B depicts polydispersity (DLS) of LNPs based on Lipid 31 to Lipid 34 in pH 7.4 HBS, pH 6.5 MBS, Post antibody (αCD3, hSP34) insertion and post freeze-thaw (−80° C.).

FIG. 7C depicts charge (Zeta potential, DLS) of LNPs based on Lipid 31 to Lipid 34 in pH 5.5 MBS, pH 7.4 HBS.

FIG. 7D depicts % RNA recovery and dye accessible RNA in LNPs based on Lipid 31 to Lipid 34.

FIG. 8A depicts diameter (DLS, nm) of LNPs based on Lipids 1, 3, 4, 5, 9, and 15 in pH 7.4 HBS, pH 6.5 MBS, Post inserted with αCD8 antibody conjugates TRX-2 and T8.

FIG. 8B depicts polydispersity (DLS) of LNPs based on Lipids 1, 3, 4, 5, 9, and 15 in pH 7.4 HBS, pH 6.5 MBS, Post inserted with αCD8 antibody conjugates TRX-2 and T8.

FIG. 9A depicts diameter (DLS, nm) of LNPs based on Lipids 1, 8, 9, 10, 11, and 15 in pH 7.4 HBS, pH 6.5 MBS, Post inserted with αCD8 antibody conjugates TRX-2 and T8.

FIG. 9B depicts polydispersity (DLS) of LNPs based on Lipids 1, 8, 9, 10, 11, and 15 in pH 7.4 HBS, pH 6.5 MBS, Post inserted with αCD8 antibody conjugates TRX-2 and T8.

FIG. 10A depicts diameter (DLS, nm) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10B depicts polydispersity (DLS) of LNPs based on Lipid 3, 4, 33, and 34 in pH 7.4 HBS, pH 6.5 MBS, Post antibody (αCD3, hSP34) insertion and post freeze-thaw (−80° C.).

FIG. 10C depicts charge (Zeta potential, DLS) of LNPs based on Lipid 3, 4, 33, and 34 in pH 5.5 MBS, pH7.4 HBS, pH6.5 MBS, post antibody (αCD3, hSP34) insertion and post freeze-thaw (−80° C.).

FIG. 10D depicts % RNA recovery and dye accessible RNA in LNPs based on Lipid 3, 4, 33, and 34.

FIG. 11A depicts GFP expression in primary human T-cells; transfected by αCD8 (hsp34) targeted LNPs based on ALC-0315, DLin-MC3-DMA, Lipid 3, Lipid 6, and Lipid 7, stored at 4° C.; % GFP+ T cells at 24 hours.

FIG. 11B depicts GFP expression in primary human T-cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); % GFP+ T cells at 24 hours.

FIG. 11C depicts GFP expression in primary human T-cells; transfected by αCD3 (hsp34) targeted LNPs based on ALC-0315, DLin-MC3-DMA, Lipid 3, Lipid 6, and Lipid 7, stored at 4° C.; GFP MFI in live T-cells at 24 hours.

FIG. 11D depicts GFP expression in primary human T-cells; transfected by αCD3 (hsp34) targeted LNPs based on ALC-0315, DLin-MC3-DMA, Lipid 3, Lipid 6, and Lipid 7, after 1 freeze-thaw cycle (−80° C. storage); GFP MFI in live T-cells at 24 hours.

FIG. 11E depicts % live T-cells transfected by αCD3 (hsp34) targeted LNPs based on ALC-0315, DLin-MC3-DMA, Lipid 3, Lipid 6, and Lipid 7, after 1 freeze-thaw cycle (−80° C. storage); % live T-cells at 24 hours.

FIG. 12A depicts GFP expression in primary human T-cells; transfected by αCD3 (hsp34) targeted LNPs based on SM-102, DLin-KC2-DMA, Lipid 3, Lipid 4 stored at 4° C.; % GFP+ T cells at 24 hours.

FIG. 12B depicts GFP expression in primary human T-cells; transfected by αCD3 (hsp34) targeted LNPs based on SM-102, DLin-KC2-DMA, Lipid 3, Lipid 4, after 1 freeze-thaw cycle (−80° C. storage); % GFP+ T cells at 24 hours.

FIG. 12C depicts GFP expression in primary human T-cells; transfected by αCD3 (hsp34) targeted LNPs based on SM-102, DLin-KC2-DMA, Lipid 3, Lipid 4, stored at 4° C.; GFP MFI in live T-cells at 24 hours.

FIG. 12D depicts GFP expression in primary human T-cells; transfected by αCD3 (hsp34) targeted LNPs based on SM-102, DLin-KC2-DMA, Lipid 3, Lipid 4, after 1 freeze-thaw cycle (−80° C. storage); GFP MFI in live T-cells at 24 hours.

FIG. 12E depicts % live T-cells transfected with by αCD3 (hsp34) targeted LNPs based on SM-102, DLin-KC2-DMA, Lipid 3, Lipid 4, after 1 freeze-thaw cycle (−80° C. storage); % live T-cells at 24 hours.

FIG. 13A depicts GFP expression in primary human T-cells; transfected by targeted LNPs based on DLin-KC2-DMA (−80° C. stored), Lipid 1 (4° C. stored), Lipid 3 (4° C. stored), and Lipid 5 (4° C. stored); % GFP+ T cells.

FIG. 13B depicts GFP expression in primary human T-cells; transfected by targeted LNPs based on DLin-KC2-DMA, Lipid 1, Lipid 3, and Lipid 5 after freeze-thaw cycle (−80° C. storage); % GFP+ T cells.

FIG. 13C depicts GFP expression in primary human T-cells; transfected by targeted LNPs based on DLin-KC2-DMA (−80° C. stored), Lipid 1 (4° C. stored), Lipid 3 (4° C. stored), and Lipid 5 (4° C. stored), GFP MFI in live T-cells.

FIG. 13D depicts GFP expression in primary human T-cells; transfected by a targeted LNPs based on DLin-KC2-DMA, Lipid 1, Lipid 3, and Lipid 5 after freeze-thaw cycle (−80° C. storage); GFP MFI in live T-cells.

FIG. 13E depicts % live T-cells transfected with targeted LNPs based on DLin-KC2-DMA, Lipid 1, Lipid 3, and Lipid 5 stored at −80° C.

FIG. 14A depicts GFP expression in primary human T-cells; transfected by αCD3 (hSP34) targeted LNPs based on DLin-KC2-DMA, Lipid 1 (4° C. stored), Lipid 8 (4° C. stored), and Lipid 8 (−80° C. stored); % GFP+ T cells.

FIG. 14B depicts GFP expression in primary human T-cells; transfected by αCD3 (hSP34) targeted LNPs based on DLin-KC2-DMA, Lipid 1 (4° C. stored), Lipid 8 (4° C. stored), and Lipid 8 (−80° C. stored); GFP MFI in live T-cells.

FIG. 14C depicts % living cells with targeted LNPs based on DLin-KC2-DMA, Lipid 1 (4° C. stored), Lipid 8 (4° C. stored), and Lipid 8 (−80° C. stored).

FIG. 15A depicts GFP expression in primary human T-cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); % GFP+ T cells.

FIG. 15B depicts GFP expression in primary human T-cells; transfected by αCD3 (hsp34) targeted LNPs based on DLin-KC2-DMA (−80° C. stored), Lipid 8 (−80° C. stored) and Lipid 10 (−80° C. stored); % GFP+ T cells.

FIG. 15C depicts GFP expression in primary human T-cells; transfected by αCD3 (hsp34) targeted LNPs based on DLin-KC2-DMA (−80° C. stored), Lipid 8 (4° C. stored), Lipid 9 (4° C. stored), and Lipid 10 (4° C. stored); GFP MFI in live T-cells.

FIG. 15D depicts GFP expression in primary human T-cells; transfected by αCD3 (hsp34) targeted LNPs based on DLin-KC2-DMA (−80° C. stored), Lipid 8 (−80° C. stored) and Lipid 10 (−80° C. stored); GFP MFI in live T-cells.

FIG. 15E depicts % live T-cells transfected with αCD3 (hsp34) targeted LNPs based on DLin-KC2-DMA (−80° C. stored), Lipid 8 (4° C. stored), Lipid 9 (4° C. stored), and Lipid 10 (4° C. stored); % live T-cells.

FIG. 15F depicts % live T-cells transfected with αCD3 (hsp34) targeted LNPs based on DLin-KC2-DMA (−80° C. stored), Lipid 8 (−80° C. stored) and Lipid 10 (−80° C. stored); % live T-cells.

FIG. 16A depicts GFP expression in primary human T-cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored); % GFP+ T cells.

FIG. 16B depicts GFP expression in primary human T-cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); % GFP+ T cells.

FIG. 16C depicts GFP expression in primary human T-cells; transfected by αCD3 (hsp34) targeted LNPs based on DLin-KC2-DMA (−80° C. stored), Lipid 3 (4° C. stored), Lipid 4 (4° C. stored), Lipid 9 (4° C. stored), Lipid 15 (4° C. stored), GFP MFI in live T-cells.

FIG. 16D depicts GFP expression in primary human T-cells; transfected by αCD3 (hsp34) targeted LNPs based on DLin-KC2-DMA (−80° C. stored), Lipid 3 (−80° C. stored), Lipid 4 (−80° C. stored), Lipid 9 (−80° C. stored), Lipid 15 (−80° C. stored); GFP MFI in live T-cells.

FIG. 16E depicts % live T-cells transfected with αCD3 (hsp34) targeted LNPs based on DLin-KC2-DMA (−80° C. stored), Lipid 3 (−80° C. stored), Lipid 4 (−80° C. stored), Lipid 9 (−80° C. stored), and Lipid 15 (−80° C. stored).

FIG. 17A depicts GFP expression in primary human T-cells; transfected by αCD8 (TRX2) targeted LNPs based on Lipid 3, Lipid 4, Lipid 9, Lipid 15, and compared with the corresponding non-targeted parent LNPs; % GFP+ T cells.

FIG. 17B depicts GFP expression in primary human T-cells; transfected by αCD8 (TRX2) targeted LNPs based on Lipid 3, Lipid 4, Lipid 9, Lipid 15, and compared with the corresponding non-targeted parent LNPs; GFP MFI in live T-cells.

FIG. 17C depicts %+Dil T-cell with αCD8 (TRX2) targeted LNPs based on Lipid 3, Lipid 4, Lipid 9, Lipid 15, and compared with the corresponding non-targeted parent LNPs.

FIG. 17D depicts Dil MFI in live T-cells with αCD8 (TRX2) targeted LNPs based on Lipid 3, Lipid 4, Lipid 9, Lipid 15, and compared with the corresponding non-targeted parent LNPs.

FIG. 17E depicts % live T-cells transfected with αCD8 (TRX2) targeted LNPs based on Lipid 3, Lipid 4, Lipid 9, Lipid 15, and compared with the corresponding non-targeted parent LNPs.

FIG. 18A depicts GFP expression in primary human T-cells; transfected by αCD8 (T8) targeted LNPs based on Lipid 3, Lipid 4, Lipid 9, Lipid 15, and compared with the corresponding non-targeted parent LNPs; % GFP+ T cells.

FIG. 18B depicts GFP expression in primary human T-cells; transfected by αCD8 (T8) targeted LNPs based on Lipid 3, Lipid 4, Lipid 9, Lipid 15, and compared with the corresponding non-targeted parent LNPs; GFP MFI in live T-cells.

FIG. 18C depicts %+Dil T-cell with αCD8 (T8) targeted LNPs based on Lipid 3, Lipid 4, Lipid 9, Lipid 15, and compared with the corresponding non-targeted parent LNPs.

FIG. 18D depicts Dil MFI in live T-cells with αCD8 (T8) targeted LNPs based on Lipid 3, Lipid 4, Lipid 9, Lipid 15, and compared with the corresponding non-targeted parent LNPs.

FIG. 18E depicts % live T-cells transfected with αCD8 (T8) targeted LNPs based on Lipid 3, Lipid 4, Lipid 9, Lipid 15, and compared with the corresponding non-targeted parent LNPs.

FIG. 19A depicts GFP expression in primary human T-cells; transfected by αCD3 (hSP34) targeted LNPs based on DLin-KC2-DMA, Lipid 2, Lipid 3, Lipid 31, and Lipid 32; stored at 4° C.; % GFP+ T cells.

FIG. 19B depicts GFP expression in primary human T-cells; transfected by αCD3 (hSP34) targeted LNPs based on DLin-KC2-DMA, Lipid 2, Lipid 3, Lipid 31, and Lipid 32; stored at 4° C.; GFP MFI in live T-cells.

FIG. 19C depicts % living T-cells with αCD3 (hSP34) targeted LNPs based on DLin-KC2-DMA, Lipid 2, Lipid 3, Lipid 31, and Lipid 32; stored at 4° C.

FIG. 20A depicts GFP expression in primary human T-cells; transfected with αCD3 (hSP34) targeted LNPs based on DLin-KC2-DMA (−80° C. stored), Lipid 3 (4° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); % GFP+ T cells.

FIG. 20B depicts GFP expression in primary human T-cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); % GFP+ T cells.

FIG. 20C depicts GFP expression in primary human T-cells; transfected with αCD3 (hSP34) targeted LNPs based on DLin-KC2-DMA (−80° C. stored), Lipid 3 (−80° C. stored), Lipid 33 (4° C. stored), Lipid 34 (4° C. stored), or transfected with αCD8 (muOKT8) targeted LNPs based on Lipid 33 (4° C. stored) Lipid 34 (4° C. stored); GFP MFI in live T-cells.

FIG. 20D depicts GFP expression in primary human T-cells; transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored); GFP MFI in live T-cells.

FIG. 20E depicts % live T-cells transfected with αCD3 (hSP34) targeted LNPs based on DLin-KC2-DMA (−80° C. stored), Lipid 3 (−80° C. stored), Lipid 33 (−80° C. stored), Lipid 34 (−80° C. stored), or transfected with αCD8 (muOKT8) targeted LNPs based on Lipid 33 (4° C. stored) and Lipid 34 (4° C. stored).

FIG. 21A depicts % αCD20 (TTR-023) CAR+ T-cells transfected by αCD3 (hSP34) targeted LNPs based on Lipid 3 (4° C. stored), Lipid 4 (4° C. stored), Lipid 9 (4° C. stored), and Lipid 33 (4° C. stored); as illustrated by % M1 value.

FIG. 21B depicts % αCD20 (TTR-023) CAR+ T-cells transfected by αCD3 (hSP34) targeted LNPs based on Lipid 3 (−80° C. stored), Lipid 4 (−80° C. stored), Lipid 9 (−80° C. stored), Lipid 33 (−80° C. stored); as illustrated by % M1 value.

FIG. 21C depicts % αCD20 (TTR-023) CAR MFI in T-cells transfected by αCD3 (hSP34) targeted LNPs based on Lipid 3 (4° C. stored), Lipid 4 (4° C. stored), Lipid 9 (4° C. stored), and Lipid 33 (4° C. stored).

FIG. 21D depicts % αCD3 (hSP34) CAR MFI in T-cells transfected by αCD3 (hSP34) targeted LNPs based on Lipid 3 (−80° C. stored), Lipid 4 (−80° C. stored), Lipid 9 (−80° C. stored), Lipid 33 (−80° C. stored).

FIG. 21E depicts % live T-cells transfected with αCD3 (hSP34) targeted LNPs based on Lipid 3 (4° C. stored), Lipid 4 (4° C. stored), Lipid 9 (4° C. stored), and Lipid 33 (4° C. stored).

FIG. 21F depicts % live T-cells transfected with αCD3 (hSP34) targeted LNPs based on Lipid 3 (−80° C. stored), Lipid 4 (−80° C. stored), Lipid 9 (−80° C. stored), Lipid 33 (−80° C. stored).

FIG. 22A depicts % αCD20 (TTR-023) CAR+ T-cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4-% M1 value.

FIG. 22B depicts αCD20 (TTR-023) CAR MFI in T-cells (CD8 population) with αCD8 (T8) targeted LNPs based on Lipid 3 (4° C. stored), Lipid 4 (4° C. stored), Lipid 9 (4° C. stored), Lipid 33 (4° C. stored), as illustrated by CD4-M1 MFI value.

FIG. 22C depicts αCD20 (TTR-023) CAR level in CD4+ T-cells transfected with αCD8 (T8) targeted LNPs based on Lipid 3 (4° C. stored), Lipid 4 (4° C. stored), Lipid 9 (4° C. stored), Lipid 33 (4° C. stored); as illustrated by the M1% value.

FIG. 22D depicts αCD20 (TTR-023) CAR level in CD4+ T-cells transfected with αCD8 (T8) targeted LNPs based on DLin-KC2-DMA (−80° C. stored), Lipid 3 (−80° C. stored), Lipid 33 (−80° C. stored), Lipid 34 (−80° C. stored); as illustrated by the M1 MFI value.

FIG. 22E depicts % live T-cells (CD4/CD8 populations) transfected with αCD8 (T8) targeted LNPs based on Lipid 3 (4° C. stored), Lipid 4 (4° C. stored), Lipid 9 (4° C. stored), Lipid 33 (4° C. stored).

FIG. 23A depicts % αCD20 (TTR-023) CAR+ T-cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4-% M1 value.

FIG. 23B depicts αCD20 (TTR-023) CAR MFI in T-cells (CD8 population) transfected with αCD8 (T8) targeted LNPs based on Lipid 3 (−80° C. stored), Lipid 4 (−80° C. stored), Lipid 9 (−80° C. stored), Lipid 33 (−80° C. stored) after one Freeze-Thaw cycle, as illustrated by CD4-M1 MFI value.

FIG. 23C depicts αCD20 (TTR-023) CAR level in CD4+ T-cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+% M1 value.

FIG. 23D depicts αCD20 (TTR-023) CAR level in CD4+ T-cells transfected with αCD8 (T8) targeted LNPs based on Lipid 3 (−80° C. stored), Lipid 4 (−80° C. stored), Lipid 9 (−80° C. stored), Lipid 33 (−80° C. stored); as illustrated by CD4+M1 MFI value.

FIG. 23E depicts % live T-cells transfected with αCD8 (T8) targeted LNPs based on Lipid 3 (−80° C. stored), Lipid 4 (−80° C. stored), Lipid 9 (−80° C. stored), Lipid 33 (−80° C. stored).

FIG. 24A depicts GFP expression in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24B depicts GFP expression in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24C depicts GFP expression in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % GFP+ T cells.

FIG. 24D depicts GFP expression in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by GFP MFI.

FIG. 24E depicts % Dil+CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T-cells.

FIG. 24F depicts Dil MFI in CD8+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 24G depicts % Dil+CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by % Dil+ T-cells.

FIG. 24H depicts Dil MFI in CD4+ T-cells transfected with αCD3 (hSP34) targeted or αCD8 (TRX2) targeted LNPs based on Lipid 9, Lipid 15, or DLin-KC3-DMA compared to vector control (mutOKT8) and un-transfected; as illustrated by Dil MFI.

FIG. 25A depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+NK cells.

FIG. 25B depicts GFP expression in NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25C depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ granulocytes.

FIG. 25D depicts GFP expression in granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 25E depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % GFP+ B cells.

FIG. 25F depicts GFP expression in B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by GFP MFI.

FIG. 26A depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+NK cells.

FIG. 26B depicts LNP binding to NK cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26C depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ granulocytes.

FIG. 26D depicts LNP binding to granulocytes in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 26E depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by % Dil+ B cells.

FIG. 26F depicts LNP binding to B cells in whole blood samples transfected with αCD3 (hSP34) targeted or αCD8 (TRX2) targeted LNPs based on Lipid 9, Lipid 15, or DLin-KC3-DMA compared to non-binding control (mutOKT8) and un-transfected; as illustrated by Dil MFI.

FIG. 27A depicts % live T-cells 24 hours after being transfected with αCD8 (TRX2) targeted LNPs expressing αCD20 (TTR-023) CAR or mCherry based on Lipid 9 or DLin-KC3-DMA.

FIG. 27B depicts % of CD8 (CD4−) T-cells expressing M1 (TRR-023) CAR after being transfected with αCD8 (TRX2) targeted LNPs expressing αCD20 (TTR-023) CAR or mCherry based on Lipid 9 or DLin-KC3-DMA.

FIG. 27C depicts M1 (TTR-023) expression Mean Fluorescence Intensity (MFI) in CD8 (CD4−) T-cells transfected with αCD8 (TRX2) targeted LNPs expressing αCD20 (TTR-023) CAR or mCherry based on Lipid 9 or DLin-KC3-DMA.

FIG. 27D depicts % of CD8 (CD4−) T-cells with mCherry expression after being transfected with αCD8 (TRX2) targeted LNPs expressing αCD20 (TTR-023) CAR or mCherry based on Lipid 9 or DLin-KC3-DMA.

FIG. 27E depicts mCherry expression Mean Fluorescence Intensity (MFI) in CD8 (CD4−) T-cells transfected with αCD8 (TRX2) targeted LNPs expressing αCD20 (TTR-023) CAR or mCherry based on Lipid 9 or DLin-KC3-DMA.

FIG. 27F depicts % of CD4+ T-cells with M1 (TTR-023) CAR expression after being transfected with αCD8 (TRX2) targeted LNPs expressing αCD20 (TTR-023) CAR or mCherry based on Lipid 9 or DLin-KC3-DMA.

FIG. 27G depicts M1 (TTR-023) expression Mean Fluorescence Intensity (MFI) in CD4+ T-cells after being transfected with αCD8 (TRX2) targeted LNPs expressing αCD20 (TTR-023) CAR or mCherry based on Lipid 9 or DLin-KC3-DMA.

FIG. 27H depicts % of CD4+ T-cells with mCherry expression after being transfected with αCD8 (TRX2) targeted LNPs expressing αCD20 (TTR-023) CAR or mCherry based on Lipid 9 or DLin-KC3-DMA.

FIG. 27I depicts % dead Raji cells in Raji (B-cell) co-culture experiment with CAR-T generated using αCD8 (TRX2) targeted LNPs expressing αCD20 (TTR-023) CAR or mCherry based on Lipid 9 or DLin-KC3-DMA.

FIG. 28A depicts % of dead Raji cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector: target ratio of 1:1, 4:1, and 8:1.

FIG. 28B depicts % of live CD8 (CD4−) T-cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector: target ratio of 1:1, 4:1, and 8:1.

FIG. 28C depicts % of live CD4+ T-cells in Raji (B-cell) co-culture experiment with CAR-T cells generated using αCD8 (TRX2) targeted LNPs expressing αCD20 (TTR-023) CAR or mCherry based on Lipid 9 or DLin-KC3-DMA, with an effector: target ratio of 1:1, 4:1, and 8:1.

FIG. 29A depicts % live T-cells 24 hours after being transfected with αCD8 (TRX2) targeted LNPs expressing αCD20 (TTR-023) CAR or mCherry based on Lipid 15 or DLin-KC3-DMA.

FIG. 29B depicts % of CD8 (CD4−) T-cells expressing M1 (TRR-023 CAR) after being transfected with αCD8 (TRX2) targeted LNPs expressing αCD20 (TTR-023) CAR or mCherry based on Lipid 15 or DLin-KC3-DMA.

FIG. 29C depicts M1 (TTR-023 CAR) expression Mean Fluorescence Intensity (MFI) in CD8 (CD4−) T-cells transfected with αCD8 (TRX2) targeted LNPs expressing αCD20 (TTR-023) CAR or mCherry based on Lipid 15 or DLin-KC3-DMA.

FIG. 29D depicts % of CD8 (CD4−) T-cells with mCherry expression after being transfected with αCD8 (TRX2) targeted LNPs expressing αCD20 (TTR-023) CAR or mCherry based on Lipid 15 or DLin-KC3-DMA.

FIG. 29E depicts mCherry expression Mean Fluorescence Intensity (MFI) in CD8 (CD4−) T-cells transfected with αCD8 (TRX2) targeted LNPs expressing αCD20 (TTR-023) CAR or mCherry based on Lipid 15 or DLin-KC3-DMA.

FIG. 30A depicts % of dead Raji cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector: target ratio of 0.31:1, 1:1, 3.16:1, 10:1, and 31.6:1.

FIG. 30B depicts % of live CD8 (CD4−) T-cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector: target ratio of 0.31:1, 1:1, 3.16:1, 10:1, and 31.6:1.

FIG. 30C depicts % of live CD4+ T-cells in Raji (B-cell) co-culture experiment with CAR-T cells generated using αCD8 (TRX2) targeted LNPs expressing αCD20 (TTR-023) CAR or mCherry based on Lipid 15 or DLin-KC3-DMA, with an effector: target ratio of 0.31:1, 1:1, 3.16:1, 10:1, and 31.6:1.

FIG. 31 depicts structures of various Fab, VHH (Nb), ScFv, Fab-ScFv and Fab-VHH hybrids.

FIG. 32A depicts GFP expression in T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % GFP+ T cells.

FIG. 32B depicts GFP expression in T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by GFP MFI.

FIG. 32C depicts % Dil+ T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by % Dil+ T-cells.

FIG. 32D depicts Dil MFI in live T-cells transfected with αCD3 targeted LNPs based on Lipid 9, Lipid 10, Lipid 13, Lipid 15, or DLin-KC2-DMA, stored at either 4° C. or post freeze-thaw (−80° C. stored), as illustrated by Dil % MFI.

FIG. 32E depicts % live T-cells transfected with αCD3 targeted LNPs based on Lipid 9, Lipid 10, Lipid 13, Lipid 15, or DLin-KC2-DMA, stored at either 4° C. or post freeze-thaw (−80° C. stored).

FIG. 33A to FIG. 33C depict % GFP+ T-cells (CD4 and CD8 populations) in Blood (FIG. 33A), Spleen (FIG. 33B), and Liver (FIG. 33C) samples (analyzed for additional cell types of interest per legend) at 24 hours post injection of GFP RNA using Lipid 15, DLin-KC3-DMA, and Lipid 9 LNP formulations and α-CD8 targeting with TRX-2 antibody.

FIG. 34A to FIG. 34C depict % DiI+ T-cells (CD4 and CD8 populations) in Blood (FIG. 34A), Spleen (FIG. 34B), and Liver (FIG. 34C) samples (analyzed for additional cell types of interest per legend) at 24 hours post injection of GFP RNA using Lipid 15 (DiI-dye labelled), DLin-KC3-DMA (No DiI-dye label used), and Lipid 9 LNP (DiI-dye labelled) formulations and α-CD8 targeting with TRX-2 antibody.

FIG. 35A depicts % DiI+CD8+ T-cells transfected with αCD8 targeted LNPs based on Lipids 10, 15, 16, 24, and 26 and ALC-0315 as a comparator, as illustrated by % DiI+ T-cells.

FIG. 35B depicts DiI MFI of CD8+ T-cells transfected with αCD8 targeted LNPs based on Lipids 10, 15, 16, 24, and 26 and ALC-0315 as a comparator, as illustrated by DiI MFI.

FIG. 35C depicts % GFP+CD8+ T-cells transfected with αCD8 targeted LNPs based on Lipids 10, 15, 16, 24, and 26 and ALC-0315 as a comparator, as illustrated by % GFP+ T-cells.

FIG. 35D depicts GFP MFI of CD8+ T-cells transfected with αCD8 targeted LNPs based on Lipids 10, 15, 16, 24, and 26 and ALC-0315 as a comparator, as illustrated by GFP MFI.

FIG. 36-1A depicts the viability of CD8+ T-cells transfected with αCD8 targeted LNPs based on DLn-KC2-DMA, as illustrated by % Live T-cells.

FIG. 36-1B depicts % DiI+CD8+ T-cells transfected with αCD8 targeted LNPs based on DLn-KC2-DMA, as illustrated by % DiI+ T-cells.

FIG. 36-1C depicts DiI MFI in CD8+ T-cells transfected with αCD8 targeted LNPs based on DLn-KC2-DMA, as illustrated by DiI MFI.

FIG. 36-1D depicts GFP expression in CD8+ T-cells transfected with αCD8 targeted LNPs based on DLn-KC2-DMA, as illustrated by % GFP+ T-cells.

FIG. 36-1E depicts GFP MFI in CD8+ T-cells transfected with αCD8 targeted LNPs based on DLn-KC2-DMA, as illustrated by GFP MFI.

FIG. 36-2A depicts % DiI+CD8+ T-cells transfected with αCD8 targeted LNPs based on DLn-KC2-DMA, as illustrated by % DiI+ T-cells.

FIG. 36-2B depicts DiI MFI in CD8+ T-cells transfected with αCD8 targeted LNPs based on DLn-KC2-DMA, as illustrated by DiI MFI.

FIG. 36-2C depicts GFP expression in CD8+ T-cells transfected with αCD8 targeted LNPs based on DLn-KC2-DMA, as illustrated by % GFP+ T-cells.

FIG. 36-2D depicts GFP MFI in CD8+ T-cells transfected with αCD8 targeted LNPs based on DLn-KC2-DMA, as illustrated by GFP MFI.

FIG. 36-3A depicts CAR expression in CD3+ T-cells of isolated CD3+ T-cells transfected with αCD8 and αCD4 dual-targeted LNPs based on Lipid 15, as illustrated by % CAR+ of CD3+ in CD3 T-cells.

FIG. 36-3B depicts CAR MFI in CD3+ T-cells of isolated CD3+ T-cells transfected with αCD8 and αCD4 dual-targeted LNPs based on Lipid 15, as illustrated by CAR MFI of CD3+ in CD3 T-cells.

FIG. 36-3C depicts CAR expression in CD4+ T-cells of isolated CD3+ T-cells transfected with αCD8 and αCD4 dual-targeted LNPs based on Lipid 15, as illustrated by % CAR+ of CD4+ in CD3 T-cells.

FIG. 36-3D depicts CAR MFI in CD4+ T-cells of isolated CD3+ T-cells transfected with αCD8 and αCD4 dual-targeted LNPs based on Lipid 15, as illustrated by CAR MFI of CD4+ in CD3 T-cells.

FIG. 36-3E depicts CAR expression in CD8+ T-cells of isolated CD3+ T-cells transfected with αCD8 and αCD4 dual-targeted LNPs based on Lipid 15, as illustrated by % CAR+ of CD8+ in CD3 T-cells.

FIG. 36-3F depicts CAR MFI in CD8+ T-cells of isolated CD3+ T-cells transfected with αCD8 and αCD4 dual-targeted LNPs based on Lipid 15, as illustrated by CAR MFI of CD8+ in CD3 T-cells.

FIG. 36-3G depicts CAR expression in CD4+ T-cells of isolated CD4+ T-cells transfected with αCD8 and αCD4 dual-targeted LNPs based on Lipid 15, as illustrated by % CAR+ of CD4+ in CD4 T-cells.

FIG. 36-3H depicts CAR MFI in CD4+ T-cells of isolated CD4+ T-cells transfected with αCD8 and αCD4 dual-targeted LNPs based on Lipid 15, as illustrated by CAR MFI of CD4+ in CD4 T-cells.

FIG. 36-3I depicts CAR expression in CD8+ T-cells of isolated CD8+ T-cells transfected with αCD8 and αCD4 dual-targeted LNPs based on Lipid 15, as illustrated by % CAR+ of CD8+ in CD8 T-cells.

FIG. 36-3J depicts CAR MFI in CD8+ T-cells of isolated CD8+ T-cells transfected with αCD8 and αCD4 dual-targeted LNPs based on Lipid 15, as illustrated by CAR MFI of CD8+ in CD8 T-cells.

FIG. 37A depicts Dil expression in CD8+ T-cells transfected with αCD3- and αCD8-targeted LNPs based on Lipid 15, at mRNA doses of 3, 1, 0.33, 0.11, and 0.037 μg/mL, as illustrated by % DiI+ T-cells.

FIG. 37B depicts DiI MFI in CD8+ T-cells transfected with αCD3- and αCD8-targeted LNPs based on Lipid 15, at mRNA doses of 3, 1, 0.33, 0.11, and 0.037 μg/mL, as illustrated by DiI MFI.

FIG. 37C depicts GFP expression in CD8+ T-cells transfected with αCD3- and αCD8-targeted LNPs based on Lipid 15, at mRNA doses of 3, 1, 0.33, 0.11, and 0.037 μg/mL, as illustrated by % GFP+ T-cells.

FIG. 37D depicts GFP MFI in CD8+ T-cells transfected with αCD3- and αCD8-targeted LNPs based on Lipid 15, at mRNA doses of 3, 1, 0.33, 0.11, and 0.037 μg/mL, as illustrated by GFP MFI.

FIG. 38A depicts GFP expression in CD8+ T-cells transfected with αCD3- and αCD8-targeted LNPs based on Lipid 15, at various time points, as illustrated by Green Integrated Intensity.

FIG. 38B depicts level of LNP association (Dil signal) in CD8+ T-cells transfected with αCD3- and αCD8-targeted LNPs based on Lipid 15, at various time points, as illustrated by NIR Integrated Intensity.

FIG. 39A depicts level of LNP association (Dil signal) in CD8+ T-cells transfected with DLin-KC3-DMA (KC3) LNPs containing different densities of αCD8 (TRX2 and 15C01), at various dose levels, as illustrated by % DiI+CD8+ T-cells.

FIG. 39B depicts level of LNP association (Dil signal) in CD8+ T-cells transfected with KC3 LNPs containing different densities of αCD8 (TRX2 and 15C01), at various dose levels, as illustrated by DiI MFI of CD8+ T-cells.

FIG. 39C depicts level of GFP expression in CD8+ T-cells transfected with KC3 LNPs containing different densities of αCD8 (TRX2 and 15C01), at various dose levels, as illustrated by % GFP+CD8+ T-cells.

FIG. 39D depicts level of GFP expression in CD8+ T-cells transfected with KC3 LNPs containing different densities of αCD8 (TRX2 and 15C01), at various dose levels, as illustrated by GFP MFI of CD8+ T-cells.

FIG. 39E depicts level of GFP expression in CD4+ T-cells transfected with KC3 LNPs containing different densities of αCD8 (TRX2 and 15C01), at various dose levels, as illustrated by % GFP+CD4+ T-cells.

FIG. 39F depicts level of GFP expression in CD4+ T-cells transfected with KC3 LNPs containing different densities of αCD8 (TRX2 and 15C01), at various dose levels, as illustrated by GFP MFI of CD4+ T-cells.

FIG. 40A depicts viability of CD8+ T-cells transfected with Lipid 15 LNPs containing different variants of the 15C01 αCD8 targeting moiety, at various densities and dose levels, as illustrated by % Live T-cells.

FIG. 40B depicts LNP association levels (Dil signal) of CD8+ T-cells transfected with Lipid 15 LNPs containing different variants of the 15C01 αCD8 targeting moiety, at various densities and dose levels, as illustrated by % DiI+ T-cells.

FIG. 40C depicts LNP association levels (Dil signal) of CD8+ T-cells transfected with Lipid 15 LNPs containing different variants of the 15C01 αCD8 targeting moiety, at various densities and dose levels, as illustrated by DiI MFI of T-cells.

FIG. 40D depicts GFP expression levels of CD8+ T-cells transfected with Lipid 15 LNPs containing different variants of the 15C01 αCD8 targeting moiety, at various densities and dose levels, as illustrated by % GFP+ T-cells.

FIG. 40E depicts GFP expression levels of CD8+ T-cells transfected with Lipid 15 LNPs containing different variants of the 15C01 αCD8 targeting moiety, at various densities and dose levels, as illustrated by GFP MFI of T-cells.

FIG. 41A depicts CD69 expression levels of CD8+ T-cells transfected with Lipid 15 LNPs containing αCD3 (SP34) or αCD8 (15C01v8 or TRX2) targeting moieties, at various dose levels, as illustrated by CD69 MFI of T-cells.

FIG. 41B illustrates a histogram of CD69 expression levels of CD8+ T-cells transfected with Lipid 15 LNPs containing αCD3 (SP34) or αCD8 (15C01v8 or TRX2) targeting moieties, at a dose of 1 μg/mL mRNA.

FIG. 42 depicts mCherry expression levels of primary NHP CD8+ T-cells transfected with Lipid 15 LNPs containing different αCD8 targeting moieties (15C01 and TRX2) as illustrated by mCherry MFI of T-cells.

FIG. 43A depicts CD22 CAR (TTR-102 (SEQ ID 294)) expression levels of primary NHP CD8+ T-cells transfected with Lipid 15 or KC3 LNPs containing the 15C01 αCD8 targeting moiety as illustrated by % CD22 CAR+ in CD8+ T-cells.

FIG. 43B depicts CD22 CAR (TTR-102 (SEQ ID 294)) expression levels of primary NHP CD8+NK cells transfected with Lipid 15 or KC3 LNPs containing the 15C01 αCD8 targeting moiety as illustrated by % CD22 CAR+ in CD8+NK cells.

FIG. 44A depicts T-cell viability of primary human CD8+ cells resulting from Lipid 15 and KC3 LNPs with an αCD8-targeting moiety (15C01 (SEQ ID 1)) after up to 5 freeze-thaw cycles as illustrated by % Live T-cells.

FIG. 44B depicts CD22 CAR (TTR-102 (SEQ ID 307)) expression of primary human CD8+ cells resulting from Lipid 15 and KC3 LNPs with an αCD8-targeting moiety (15C01 (SEQ ID 1)) after up to 5 freeze-thaw cycles as illustrated by % CD22 CAR+ T-cells.

FIG. 44C depicts CD22 CAR (TTR-102 (SEQ ID 307)) expression of primary human CD8+ cells resulting from Lipid 15 and KC3 LNPs with an αCD8-targeting moiety (15C01 (SEQ ID 1)) after up to 5 freeze-thaw cycles as illustrated by CD22 CAR MFI.

FIG. 44-1A depicts viability of primary human CD8+ cells resulting from Lipid 15 LNPs with varying DSPC content and with an &CD8-targeting moiety (15C01v8 (SEQ ID 9)) as illustrated by % Live cells.

FIG. 44-1B CAR expression (TTR-102 (SEQ ID 316)) of primary human CD8+ cells resulting from Lipid 15 LNPs with varying DSPC content and with an αCD8-targeting moiety (15C01v8 (SEQ ID 9)) as illustrated by % CAR+ cells.

FIG. 44-1C CAR expression (TTR-102 (SEQ ID 316)) of primary human CD8+ cells resulting from Lipid 15 LNPs with varying DSPC content and with an αCD8-targeting moiety (15C01v8 (SEQ ID 9)) as illustrated by CAR MFI.

FIG. 44-1D CAR expression (TTR-102 (SEQ ID 316)) of primary human CD8+ cells resulting from Lipid 15 LNPs with varying DSPC content and with an αCD8-targeting moiety (15C01v8 (SEQ ID 9)) as illustrated by % CAR+ cells at different mRNA doses.

FIG. 44-1E CAR expression (TTR-102 (SEQ ID 316)) of primary human CD8+ cells resulting from Lipid 15 LNPs with varying DSPC content and with an &CD8-targeting moiety (15C01v8 (SEQ ID 9)) as illustrated by CAR MFI at different mRNA doses.

FIG. 44-1F CAR expression (TTR-102 (SEQ ID 316)) of primary human CD8+ cells resulting from Lipid 15 LNPs with varying DSPC content and with an αCD8-targeting moiety (15C01v8 (SEQ ID 9)) as illustrated by % CAR+ cells at different time points.

FIG. 44-1G CAR expression (TTR-102 (SEQ ID 316)) of primary human CD8+ cells resulting from Lipid 15 LNPs with varying DSPC content and with an &CD8-targeting moiety (15C01v8 (SEQ ID 9)) as illustrated by CAR MFI at different time points.

FIG. 45 illustrates the general 2nd generation CAR design (top panel) consisting of an antiCD22 ScFv with a VH and VL domain connected by a linker, an extracellular hinge domain, a transmembrane domain, and an intracellular co-stimulatory domain and signaling domain. The bottom panel illustrates four different CAR cassette designs with varying extracellular hinge domains.

FIG. 46 depicts the CD22 CAR expression in HEK293T cells transfected with CAR plasmid DNA as illustrated by % CAR expression.

FIG. 47 depicts the upregulation of the early T-cell activation marker, CD69, in Jurkat cells transfected with CAR plasmid DNA and co-cultured with target-expressing Raji cells, as illustrated by the fold-over background of CD69 expression.

FIG. 48A depicts the CD22 CAR expression in Jurkat cells transfected with mRNA encoding various CD22 CAR constructs as illustrated by % CAR Expression.

FIG. 48B depicts the CD22 CAR expression in Jurkat cells transfected with mRNA encoding various CD22 CAR constructs as illustrated by CAR MFI expression.

FIG. 49A depicts the CD22 CAR expression in primary human T-cells transfected with mRNA encoding various CD22 CAR constructs as illustrated by % CAR Expression.

FIG. 49B depicts the CD22 CAR expression in primary human T-cells transfected with mRNA encoding various CD22 CAR constructs as illustrated by CAR MFI expression.

FIG. 50 depicts CAR-mediated cytotoxicity of Raji cells co-cultured with human T-cells transfected with mRNA encoding various CD22 CAR constructs by electroporation as illustrated by % Dead Raji cells.

FIG. 51A depicts CAR-mediated cytotoxicity of Nalm6 cells co-cultured with human T-cells transfected with mRNA encoding various CD22 CAR constructs by electroporation as illustrated by % Dead Nalm6 cells.

FIG. 51B depicts CAR-mediated cytotoxicity of K562 cells co-cultured with human T-cells transfected with mRNA encoding various CD22 CAR constructs by electroporation as illustrated by % Dead K562 cells.

FIG. 52 depicts the epitope binning of anti-CD22 binders against CD22.

FIG. 53 depicts the fraction of T-cells transfected with mRNA encoding various CD22 CAR constructs binding human CD22 antigen and Rhesus CD22 antigen, receptively, as illustrated by % CD22 CAR+ cells.

FIG. 54 depicts the CD22 CAR protein expression levels over a time course of 120 hours in primary human T-cells resulting from CAR mRNA transfection, as illustrated by % CD22 CAR+ cells.

FIG. 55 depicts the level of cytokine secretion (TNF-alpha, IFN-gamma, Granzyme A, Granzyme B, and GM-CSF) in primary human T-cells transfected with Lipid 15 LNPs containing antiCD8 (15C01) targeting moiety and encapsulating various CAR mRNA constructs as illustrated by the level of cytokine secreted in pg/mL.

FIG. 56 depicts the levels of CD22 expression on various cancer cell lines as illustrated by CD22 receptors per cell.

FIG. 57A depicts the level of cytotoxicity resulting from T-cells transfected with Lipid 15 LNPs with inserted antiCD8 (15C01v8)-targeting moiety encapsulating various CAR-encoding mRNA payloads and co-cultured with wild-type Nalm6 cells, as illustrated by % Dead Nalm6 WT cells.

FIG. 57B depicts the level of cytotoxicity resulting from T-cells transfected with Lipid 15 LNPs with inserted antiCD8 (15C01v8)-targeting moiety encapsulating various CAR-encoding mRNA payloads and co-cultured with CD22 knockout (KO) Nalm6 cells, as illustrated by % Dead Nalm6 CD22KO cells.

FIG. 57C depicts the level of cytotoxicity resulting from T-cells transfected with Lipid 15 LNPs with inserted antiCD8 (15C01v8)-targeting moiety encapsulating various CAR-encoding mRNA payloads and co-cultured with wild-type Raji cells, as illustrated by % Dead Raji WT cells.

FIG. 57D depicts the level of cytotoxicity resulting from T-cells transfected with Lipid 15 LNPs with inserted antiCD8 (15C01v8)-targeting moiety encapsulating various CAR-encoding mRNA payloads and co-cultured with CD22 knockout (KO) Raji cells, as illustrated by % Dead Raji CD22KO cells.

FIG. 57E depicts the level of cytotoxicity resulting from T-cells transfected with Lipid 15 LNPs with inserted antiCD8 (15C01v8)-targeting moiety encapsulating various CAR-encoding mRNA payloads and co-cultured with Daudi cells, as illustrated by % Dead Daudi cells.

FIG. 57F depicts the level of cytotoxicity resulting from T-cells transfected with Lipid 15 LNPs with inserted antiCD8 (15C01v8)-targeting moiety encapsulating various CAR-encoding mRNA payloads and co-cultured with K562 cells, as illustrated by % Dead K562 cells.

FIG. 57G depicts the level of cytotoxicity resulting from T-cells transfected with Lipid 15 LNPs with inserted antiCD8 (15C01v8)-targeting moiety encapsulating various CAR-encoding mRNA payloads and co-cultured with JVM-2 cells, as illustrated by % Dead JVM-2 cells.

FIG. 57H depicts the level of cytotoxicity resulting from T-cells transfected with Lipid 15 LNPs with inserted antiCD8 (15C01v8)-targeting moiety encapsulating various CAR-encoding mRNA payloads and co-cultured with Reh cells, as illustrated by % Dead Reh cells.

FIG. 58A depicts the CD22 CAR protein expression in primary human CD8+ T-cells resulting from Lipid 15 LNPs with an antiCD8-targeting moiety (15C01v8) encapsulating various mRNA-encoding CARs or reporter protein (mCherry), as illustrated by CD22 CAR MFI.

FIG. 58B depicts the mCherry protein expression in primary human CD8+ T-cells resulting from Lipid 15 LNPs with an antiCD8-targeting moiety (15C01v8 (SEQ ID 9)) encapsulating various mRNA-encoding CARs (SEQ ID 316 and SEQ ID 313) or reporter protein (mCherry), as illustrated by % mCherry+ cells.

FIG. 59A depicts repeated CAR-mediated cytotoxicity of Nalm6 target cells resulting from co-culture with CD8+ T-cells after transfection with Lipid 15 LNPs inserted with a CD8-targeting moiety (15C01 (SEQ ID 1)) and encapsulating CD22 CAR mRNA (TTR-102 (SEQ ID 307)), as illustrated by % Nalm6 cell (normalized to T=0). Nalm6 cells were added to the culture every 48 hours and LNPs were added every 96 hours.

FIG. 59B depicts repeated CAR-mediated cytotoxicity of Nalm6 target cells resulting from co-culture with CD8+ T-cells after transfection with Lipid 15 LNPs inserted with a CD8-targeting moiety (15C01 (SEQ ID 1)) and encapsulating CD22 CAR mRNA (TTR-102 (SEQ ID 307)), as illustrated by % Nalm6 cell (normalized to T=0). Nalm6 cells were added to the culture every 96 hours.

FIG. 60A depicts CD22 CAR expression in T-cells after transfection with anti-CD8 (15C01 (SEQ ID 1)) targeted LNPs based on Lipid 15 encapsulating mRNA encoding various UTR- and codon-optimized variants of TTR-102 (SEQ ID 307 and SEQ ID 316) and TTR-121 (SEQ ID 315 and SEQ ID 317), respectively, as illustrated by % CAR+ cells.

FIG. 60B depicts CD22 CAR expression in T-cells after transfection with anti-CD8 (15C01 (SEQ ID 1)) targeted LNPs based on Lipid 15 encapsulating mRNA encoding various UTR- and codon-optimized variants of TTR-102 (SEQ ID 307 and SEQ ID 316) and TTR-121 (SEQ ID 315 and SEQ ID 317), respectively, as illustrated by CAR MFI.

FIG. 61A depicts CAR-mediated cytotoxicity of Raji target cells resulting from co-culture with CD8+ T-cells after transfection with Lipid 15 LNPs inserted with a CD8-targeting moiety (15C01 (SEQ ID 1)) and encapsulating mRNA encoding various UTR- and codon-optimized variants of TTR-102 (SEQ ID 307 and SEQ ID 316) and TTR-121 (SEQ ID 315 and SEQ ID 317), respectively, as illustrated by % Dead Raji cells at various effector-to-target cell ratios (E:Ts).

FIG. 61B depicts CAR-mediated cytotoxicity of Nalm6 target cells resulting from co-culture with CD8+ T-cells after transfection with Lipid 15 LNPs inserted with a CD8-targeting moiety (15C01 (SEQ ID 1)) and encapsulating mRNA encoding various UTR- and codon-optimized variants of TTR-102 (SEQ ID 307 and SEQ ID 316) and TTR-121 (SEQ ID 315 and SEQ ID 317), respectively, as illustrated by % Dead Nalm6 cells at various effector-to-target cell ratios (E:Ts).

FIG. 62A depicts in vivo CAR T expression in blood 24 hr after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown to multiple dose levels. PBMC-engrafted NSG mice were dosed at day 14 post PBMC engraftment.

FIG. 62B depicts in vivo CAR T expression in blood after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown at diverse time points after administrating one dose of LNP/mRNA at 0.3 mg/kg. PBMC-engrafted NSG mice were dosed at day 14 post PBMC engraftment.

FIG. 62C depicts in vivo CAR T expression in blood 24 hr after in vivo delivery via tail vein of LNP inserted with a CD8-Targeting moiety (15C01) and encapsulating mRNA encoding for TTR-102. % of anti-CD22 CAR T in total alive gated CD8+ T cells is shown 24 hr post administration of Dose #1 or Dose #3 at 0.3 mg/kg. PBMC-engrafted NSG mice were dosed at day 14 (Dose #1) or 21 (Dose #3) post PBMC engraftment.

FIG. 62D depicts in vivo CAR T expression in spleen 24 hr after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown to multiple dose levels. PBMC-engrafted NSG mice were dosed at day 14 post PBMC engraftment.

FIG. 62E depicts in vivo CAR T expression in bone marrow (BM) 24 hr after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown to multiple dose levels. PBMC-engrafted NSG mice were dosed at day 14 post PBMC engraftment.

FIG. 62F depicts in vivo CAR T expression in lung 24 hr after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown to multiple dose levels. PBMC-engrafted NSG mice were dosed at day 14 post PBMC engraftment.

FIG. 62G depicts in vivo CAR T expression in spleen after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown at diverse time points after administrating one dose of LNP/mRNA at 0.3 mg/kg. PBMC-engrafted NSG mice were dosed at day 14 post PBMC engraftment.

FIG. 62H depicts in vivo CAR T expression in bone marrow (BM) after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown at diverse time points after administrating one dose of LNP/mRNA at 0.3 mg/kg. PBMC-engrafted NSG mice were dosed at day 14 post PBMC engraftment.

FIG. 62I depicts in vivo CAR T expression in lung after in vivo delivery via tail vein of one single dose of LNP inserted with a CD8-Targeting moiety (15C01) and encapsulating mRNA encoding for TTR-102. % of anti-CD22 CAR T in total alive gated CD8+ T cells is shown at diverse time points after administrating one dose of LNP/mRNA at 0.3 mg/kg. PBMC-engrafted NSG mice were dosed at day 14 post PBMC engraftment.

FIG. 63A depicts in vivo CAR T function in spleen after in vivo delivery via tail vein of one single dose of LNP inserted with a CD8-Targeting moiety (15C01) and encapsulating mRNA encoding for TTR-102. B cell aplasia was used as a tool to evaluate functional CAR T persistence in vivo. % of CD19+ B cells in total alive gated hCD45+ T cells is shown at 24 hr post one single dose of LNP/mRNA at 0.3 mg/kg. PBMC-engrafted NSG mice were dosed at day 14 post PBMC engraftment.

FIG. 63B depicts in vivo CAR T function in bone marrow (BM) after in vivo delivery via tail vein of one single dose of LNP inserted with a CD8-Targeting moiety (15C01) and encapsulating mRNA encoding for TTR-102. B cell aplasia was used as a tool to evaluate functional CAR T persistence in vivo. % of CD19+ B cells in total alive gated hCD45+ T cells is shown at 24 hr post one single dose of LNP/mRNA at 0.3 mg/kg. PBMC-engrafted NSG mice were dosed at day 14 post PBMC engraftment.

FIG. 64A depicts cytokine secretion of IFN-alpha2 from MIMIC® assay when induced with CD8-targeted (15C01v8 (SEQ ID 9)) LNPs encapsulating various concentrations of dsRNA.

FIG. 64B depicts cytokine secretion of IFN-gamma from MIMIC® assay when induced with CD8-targeted (15C01v8 (SEQ ID 9)) LNPs encapsulating various concentrations of dsRNA.

FIG. 64C depicts cytokine secretion of IL-10 from MIMIC® assay when induced with CD8-targeted (15C01v8 (SEQ ID 9)) LNPs encapsulating various concentrations of dsRNA.

FIG. 64D depicts cytokine secretion of IL-6 from MIMIC® assay when induced with CD8-targeted (15C01v8 (SEQ ID 9)) LNPs encapsulating various concentrations of dsRNA.

FIG. 64E depicts cytokine secretion of MIP-1beta from MIMIC® assay when induced with CD8-targeted (15C01v8 SEQ ID 9) LNPs encapsulating various concentrations of dsRNA.

FIG. 64F depicts cytokine secretion of RANTES from MIMIC® assay when induced with CD8-targeted (15C01v8 (SEQ ID 9)) LNPs encapsulating various concentrations of dsRNA.

FIG. 64G depicts cytokine secretion of TNF-alpha from MIMIC® assay when induced with CD8-targeted (15C01v8 (SEQ ID 9)) LNPs encapsulating various concentrations of dsRNA.

FIG. 65 depicts cytokine secretion of TNF-alpha, IL-2, IFN-gamma, IL-4, MIP-1beta, IL-6, and IFN-alpha2 from MIMIC® assay when induced with CD8-targeted (15C01v8 (SEQ ID 9)) Lipid 15 LNPs or non-targeted (NT) Lipid 15 LNPs encapsulating CD22 CAR mRNA (TTR-102 (SEQ ID 307)).

FIG. 66 depicts cytokine secretion of TNF-alpha, IL-2, IFN-gamma, IL-4, MIP-1beta, IL-6, and IFN-alpha2 from MIMIC® assay when induced with CD8-targeted (15C01v8 (SEQ ID 9)) Lipid 15 LNPs encapsulating TTR-102 (SEQ ID 307), TTR-121 (SEQ ID 315), or TTR-103 (SEQ ID 306) CD22 CAR mRNAs, or mCherry mRNA.

FIG. 67 depicts cytokine secretion of TNF-alpha, IL-2, IFN-gamma, IL-4, MIP-1beta, IL-6, and IFN-alpha2 from MIMIC® assay when induced with LNPs based on Lipid 15, KC3, MC3, or Lipid 9.

FIG. 68 depicts cytokine secretion of TNF-alpha, IL-2, IFN-gamma, IL-4, MIP-1beta, IL-6, and IFN-alpha2 from MIMIC® assay when induced with targeted (15C01 (SEQ ID 1)) or non-targeted (NT) LNPs based on Lipid 15, KC3, MC3, or Lipid 9.

FIG. 69. Anion Exchange Chromatography profile of VHH 15C01-GGC after capture and subsequent polish. The product was loaded on Capto Q ImpRes (Cytiva) in a 25 mM Tris pH7.5 buffer and eluted with the same buffer with a salt gradient from 0 to 2000 mM of NaCl.

FIG. 70. Anion Exchange Chromatography profile of VHH 15C01-GGC after capture, a reduction step in TCEP and subsequent polish. The product was loaded on Capto Q ImpRes (Cytiva) in a 25 mM Tris pH7.5 buffer and eluted with the same buffer with a salt gradient from 0 to 2000 mM of NaCl.

FIG. 71 depicts concept and compositions of an exemplary targeted LNP.

FIG. 72. depicts exemplary method for making phospholipid-PEG-ISVD conjugate.

FIG. 73 depicts an example of using click chemistry to make phospholipid-PEG-ISVD conjugate.

FIG. 74 depicts an exemplary method for making targeted LNP through antibody conjugated lipid-PEG micelles and parent LNPs.

FIG. 75 depicts the concept of reprogramming CD22 CAR-T cells in vivo using targeted mRNA LNPs. Upon intravenous infusion, the LNPs engage with the receptor on the T-cells via the inserted targeting moiety. Receptor engagement results in endocytosis of the LNP followed by endosomal escape of the mRNA. The mRNA is then available for translation by the ribosome and the encoded protein, being the chimeric antigen receptor (CAR) molecule, is expressed on the surface of the T-cell. Once the CAR is expressed, the T-cell can mediate its effector function-killing of malignant CD22-expressing B-cells.

FIG. 76A to FIG. 76G depicts Lipid 15-based LNPs show highly efficient mRNA delivery to primary human T-cells in vitro and 1.5% DPG-PEG shows superior transfection in vivo. FIG. 76A) Illustration of the LNP formulation workflow. The left panel depicts how an aqueous solution containing the mRNA and an organic solution containing the various lipids are mixed by microfluidic mixing using a NanoAssemblr Ignite. The right panel depicts how a protein-based targeting moiety conjugate is post-inserted into the non-targeted LNP to form a targeted LNP. Created with BioRender.com. FIG. 76B) The fraction of DiI-positive primary human T-cells, demonstrating the level of association and binding to T-cells 24 hours after incubation with 1 μg/mL CD8-targeted LNPs formulated with different ionizable lipids and encapsulating mRNA encoding GFP. FIG. 76C) The fraction of GFP-positive T-cells and FIG. 76D) the GFP MFI evaluating the level of protein expression in T-cells 24 hours after incubation with 1 μg/mL and 0.25 μg/mL CD8-targeted LNPs formulated with different ionizable lipids and encapsulating mRNA encoding GFP. Data are mean of values in n=2 replicates. The displayed p-values are from a two-way ANOVA. FIG. 76E) Histograms of Dil and GFP values in one representative donor illustrating the differences between CD8-targeted LNPs based on Lipid 15 and ALC-0315.

FIG. 76F) In vivo comparison of 1 mg/kg CD3-targeted (SP34) LNPs based on DLin-KC2-DMA (KC2) and with 2.5% DMG-PEG, DPG-PEG, and DSG-PEG, respectively, demonstrating the level of GFP-positive cells of CD4+ and CD8+ T-cells, 24 hours after intravenous injection into humanized NSG mice. Data are from biologically independent mice (n=4 in LNP-treated groups, n=3 in vehicle group). The displayed p-values are from a two-way ANOVA. FIG. 76G) In vivo comparison of 0.3 mg/kg CD3-targeted (SP34) LNPs based on KC2 and with different percentages of DMG-PEG, DPG-PEG, and DPPE-PEG, demonstrating the level of GFP-positive cells of CD8+ T-cells, 24 hours after intravenous injection into humanized NSG mice. Data are from biologically independent mice (n=4 in LNP groups, n=2 in vehicle group). The displayed p-values are from an ordinary one-way ANOVA.

FIG. 77A to FIG. 77E depicts αCD8 ISVD-targeted LNPs show dose-dependent and specific delivery to CD8+ cells without inducing cytokine release in vitro. FIG. 77A) The fraction of DiI- and FIG. 77B) GFP-positive primary human T-cells 24 hours after incubation with 0.33 μg/mL mRNA encapsulated in targeted LNPs inserted with αCD8 (anti-CD8 Fab (TRX2) or Nb8), αCD3 (anti-CD3 Fab (SP34)), or non-binding (Fab (mutOKT8)) targeting moieties in three independent donors. Each bar represents the mean of technical replicates (n=3). The displayed p-values are from a two-way ANOVA. FIG. 77C) The level of GFP expression over time in T-cells evaluated with the Incucyte SX5 and illustrated as the Green Integrated Intensity (GCU). Data are from biologically independent replicates (n=3). FIG. 77D) Levels of IFN-γ detected in the supernatants 24 hours after incubation with 0.33 μg/mL mRNA encapsulated in targeted LNPs. Each bar represents the mean of technical replicates (n=3). FIG. 77E) Ex vivo whole blood experiment enabling investigation of expression in Granulocytes, B-cells, T-cells, and NK cells by flow cytometry (left panel). Fraction of GFP-positive cells in various cell subsets 24 hours after incubation with mRNA-encapsulating LNPs inserted with the humanized &CD8 VHH (Nb8-H3) or non-binding (Fab (mutOKT8)) targeting moieties (right panel). Data are from biologically independent replicates (n=8). Created with BioRender.com.

FIG. 78A to FIG. 78L depicts αCD8 ISVD-targeted LNPs allow for specific and functional CAR T-cell reprogramming in vitro. FIG. 78A) Illustration of the PBMC transfection workflow. PBMCs from healthy donors were seeded into a 12-well plate and rested for 2 hours before transfecting with 0.3 μg/mL mRNA encapsulated in LNPs targeted with the humanized αCD8 VHH (Nb8-H3). 4 hours after LNP addition, unbound LNPs were washed away, and cells were incubated for 24 hours until staining and analysis. Created with BioRender.com. FIG. 78B) The fraction of CD22 CAR-positive of CD8+ human T-cells and FIG. 78C) of CD4+ human T-cells, 24 hours after incubation with αCD8 (Nb8-H3) targeted LNPs encapsulating mRNA encoding a CD22 (22-V4) chimeric antigen receptor (CAR) or a non-CAR payload (LNP control).

FIG. 78D) The number of live B-cells per 100 μL of the acquired sample. Data are from biologically independent replicates (n=4). The displayed p-values are from a two-way ANOVA. CAR T-cells were co-cultured with FIG. 78E) Nalm6 wildtype (WT) and FIG. 78F) Nalm6 CD22 knock-out (KO) cancer cell lines, respectively, at increasing effector-to-target cell ratios (E:Ts) and the level of CAR-mediated cytotoxicity was evaluated 48 hours later. Data are from biologically independent replicates (n=3). The displayed p-value is from a two-tailed paired t-test comparing 22-V4 to m971. FIG. 78G) Representative ImageStream images of LNP-transfected CD8+ T-cells 24 hours after LNP addition. Staining shows CD8 and CAR expression in live single cells in 22-V4-LNP treated T-cells (left panel) and untreated T-cells (right panel).

FIG. 78H) Representative ImageStream images (left panel) of LNP-transfected CD8+ T-cells co-cultured with CTV-stained Nalm6 for 24 hours demonstrating CAR-mediated target cell binding. Staining shows CD8, CAR, and CTV in live doublets. The percent live touching doublet events of total live doublet events was quantified and compared between treatment groups (right panel). Data are mean of values in n≥2 technical replicates. The displayed p-values are from one-way ANOVA. FIG. 78I) Repeated killing of Nalm6 cells with re-addition of LNPs every 4 days and Nalm6 cell every 2 days or FIG. 78J) without re-addition of LNPs and with re-addition of Nalm6 cells every 4 days. Data are from biologically independent replicates (n=3). The displayed p-values are from two-tailed paired t-tests comparing 22-V4 to the LNP control. Created with BioRender.com. FIG. 78K) MIMIC® data showing cytokine levels in samples treated with non-CAR LNPs (LNP control) and CAR-LNPs (22-V4), respectively, and compared to PBS-treated samples. Data are from biologically independent replicates (n=6). The displayed p-values are from a two-way ANOVA. FIG. 78L) MIMIC® data showing cytokine levels in groups treated with CAR-LNPs (22-V4) containing RP-HPLC-purified CAR mRNA and 0.2% spiked in dsRNA, respectively. Data are from biologically independent replicates (n=6). The displayed p-values are from multiple paired t-tests.

FIG. 79A to FIG. 79F depicts CD8-targeted Lipid 15 LNPs enable efficient reprogramming of CAR-T cells directly in vivo. FIG. 79A) The fraction of CD22 CAR-positive primary human CD8+ T-cells in the blood, spleen, bone marrow (BM), and lungs, respectively, 24 hours after intravenous tail vein injection of 0.3 mg/kg αCD8 (Nb8-H3) targeted LNPs encapsulating mRNA encoding a CD22 (22-V4) CAR. Data are from biologically independent mice (n=3). The displayed p-values are from a two-way ANOVA. FIG. 79B) The fraction of CD22 CAR-positive primary human CD8+ T-cells in the blood 24 hours after the first (Day 2) and fourth (Day 11) dose of 0.3 mg/kg αCD8 (Nb8-H3) targeted LNPs encapsulating mRNA encoding a CD22 (22-V4) CAR. Data are from biologically independent mice (n≥3). The displayed p-values are from a two-way ANOVA. FIG. 79C) Fraction of CD19-positive cells in the spleen and BM demonstrating B-cell aplasia. Data are from biologically independent mice (n≥4). The displayed p-values are from a two-way ANOVA. FIG. 79D) The percent body weight (BW) changes after dosing twice per week. Data are from biologically independent mice (n≥3). The displayed p-values are from two-tailed paired t-tests comparing 22-V4 to the vehicle group. FIG. 79E) The spleen weight is reported as the % weight of the spleen per BW 24 hours after the fourth dose. Data are from biologically independent mice (n≥3). The displayed p-value is from an unpaired t-test. FIG. 79F) The fraction of GFP-positive cells in different subsets of the liver. Data are from biologically independent mice (n=3). The displayed p-values are from a two-way ANOVA.

FIG. 80A to FIG. 80D depicts In vivo reprogrammed CAR-T cells suppress the growth of a humanized orthotopic tumor efficacy model. FIG. 80A) Illustration of the humanized Nalm6 tumor model. Nalm6-Luc tumor cells were intravenously (IV) injected into NSG MHC I/II KO mice on Day 0. On day 6, human PBMCs were engrafted by an IV injection followed by IV administration of vehicle buffer or Nb8-H3-targeted LNPs containing mCherry or CAR-encoding mRNA (22-V4) on days 7, 11, 14, 18, and 21 and imaging by IVIS on days 6, 11, 14, 18, 21, and 26. FIG. 80B) Luciferase imaging was performed at the respective time points. FIG. 80C) Tumor growth over time as illustrated by the quantified luciferase signals shown as the average radiance (p/see/cm2/sr). Data are from biologically independent mice (n=9). The displayed p-values are from a two-way ANOVA comparing 22-V4 to the vehicle group at each time point. FIG. 80D) Immunohistochemistry (IHC) of the liver and spleen on Day 26 of the study. The arrows indicate CD22-positive tumor cells. The displayed images are from left to right, Mouse ID 1-5, Mouse ID 2-3, and Mouse ID 3-3.

FIG. 81A to FIG. 81D depicts characterization of Lipid 15-based LNPs encapsulating CAR- or GFP-encoding mRNA. FIG. 81A) Size distribution, polydispersity index (PDI), and FIG. 81B) zeta potential at pH 5.5 and pH 7.4 were measured using a Zetasizer. FIG. 81C) The RNA encapsulation efficiency was quantified using the Quant-iT RiboGreen RNA Assay Kit. Measurements were performed in triplicates. FIG. 81D) Representative CryoEM images of αCD8 (Nb8-H3) targeted Lipid 15 LNPs. 200 μM (top panel) and 500 μM scale shown (bottom panel). Created with BioRender.com.

FIG. 82A to FIG. 82F depicts Lipid chemistry of various lipids. Chemical structures of FIG. 82A) DLin-KC2-DMA (KC2-DMA), FIG. 82B) KC3-DMA, FIG. 82C) ALC-0315, FIG. 82D) SM-102, FIG. 82E) Dialkyl lipid, and FIG. 82F) phospholipid degradation.

FIG. 83A to FIG. 83C depicts chemical structures of novel ionizable lipids and PEG lipids. Chemical structures of FIG. 83A) proprietary branched lipids 1, 2, 3, 4, 5, 6, 7, 8, 9, 10, 15, 16, 24A, and 26. FIG. 83B) Chemical structures of DMG-PEG, DPG-PEG, and DSG-PEG. FIG. 83C) Chemical structures of DMPE-PEG, DPPE-PEG, and DSPE-PEG.

FIG. 84A to FIG. 84L depicts Lipid 15-based LNPs show low immunogenicity and efficient mRNA delivery after freeze-thaw. FIG. 84A-FIG. 84C) Cytokine levels (pg/mL) comparing the effect of different ionizable lipid formulations in the MIMIC® assay. Data are from biologically independent replicates (n=6). The displayed p-values are from one-way ANOVA. FIG. 84D) The fraction of DiI-positive primary human T-cells and (FIG. 84E) the DiI MFI demonstrating the level of association and binding to T-cells 24 hours after incubation with LNPs formulated with different ionizable lipids and pre-(4C) and post-freeze-thawing (FT). (FIG. 84F) The fraction of GFP-positive T-cells and (FIG. 84G) the GFP MFI evaluating the level of protein expression in T-cells 24 hours after incubation with LNPs formulated with different ionizable lipids and pre-(4C) and post-freeze-thawing (FT). (FIG. 84H) The fraction of live primary human T-cells normalized to a PBS-treated control demonstrating no significant decrease in T-cell viability 24 hours after incubation with LNPs formulated with different ionizable lipids and pre-(4C) and post-freeze-thawing (FT). Data are mean of values in n=2 replicates. The displayed p-values are from one-way ANOVA. (FIG. 84I-FIG. 84L) In vivo comparison of 1 mg/kg CD3-targeted LNPs based on KC2 and with 2.5% DMG-PEG, DPG-PEG, and DSG-PEG, respectively, demonstrating the level of GFP-positive cells of CD4+ and CD8⁺ T-cells, 24 hours after intravenous injection into humanized NSG mice in various organs. Data are from biologically independent mice (n=4 in LNP-treated groups, n=3 in vehicle group). The displayed p-values are from a two-way ANOVA.

FIG. 85A-FIG. 85B depicts size and PDI of LNPs formulated with novel ionizable lipids 1-8. FIG. 85A) Diameter (DLS, nm) of LNPs based on Lipid 1 to Lipid 8 in pH 7.4 HBS, pH 6.5 MBS, post antibody (αCD3, SP34) insertion and post freeze-thaw (−80° C.). FIG. 85B) Polydispersity (DLS) of LNPs based on Lipid 1 to Lipid 8 in pH 7.4 HBS, pH 6.5 MBS, post antibody (αCD3, SP34) insertion and post freeze-thaw (−80° C.). Data are mean of values in n=2 replicates

FIG. 86A-FIG. 86B depicts size and PDI of LNPs formulated with novel ionizable lipids 9, 10, 11, and 15. FIG. 86A) Diameter (DLS, nm) of LNPs based on Lipids 9, 10, 11, and 15 in pH 7.4 HBS, pH 6.5 MBS, post antibody (αCD3, SP34) insertion and post freeze-thaw (−80° C.). FIG. 86B) Polydispersity (DLS) of LNPs based on Lipids 9, 10, 11, and 15 in pH 7.4 HBS, pH 6.5 MBS, post antibody (αCD3, SP34) insertion and post freeze-thaw (−80° C.). Data are mean of values in n=2 replicates.

FIG. 87A-FIG. 87D depicts LNP characterization of LNPs formulated with ionizable lipids 10, 15, 16, 24A, and 26. FIG. 87A) Diameter (DLS, nm) of targeted LNPs (αCD8, Nb8 VHH) based on Lipids 10, 15, 16, 24A, 26, and ALC-0315 pre and post freeze-thaw. FIG. 87B) Polydispersity of targeted LNPs (αCD8, Nb8 VHH) based on Lipids 10, 15, 16, 24A, 26, and ALC-0315 pre and post freeze-thaw. FIG. 87C) Zeta Potential (mV) of targeted LNPs (αCD8, Nb8 VHH) based on Lipids 10, 15, 16, 24A, 26, and ALC-0315 pre and post freeze-thaw in pH 5.5 MES and pH 7.4 HBS. FIG. 87D) Total mRNA recovery (ug/mL) and % dye-accessible mRNA of targeted LNPs (αCD8, Nb8 VHH) based on Lipids 10, 15, 16, 24A, 26, and ALC-0315 pre and post freeze-thaw.

FIG. 88A-FIG. 88E depicts transfection of human T-cells with CD3-targeted LNPs based on Lipids 1, 3, and 5 pre- and post freeze-thaw. FIG. 88A) % GFP+ primary human T-cells after transfection with CD3-targeted LNPs based on DLn-KC2-DMA (−80° C. stored), Lipid 1 (4° C. stored), Lipid 3 (4° C. stored), and Lipid 5 (4° C. stored). FIG. 88B) % GFP+ primary human T-cells after transfection with CD3-targeted LNPs based on DLn-KC2-DMA, Lipid 1, Lipid 3, and Lipid 5 after freeze-thaw cycle (−80° C. storage). FIG. 88C) GFP MFI in primary human T-cells after transfection with CD3-targeted LNPs based on DLn-KC2-DMA (−80° C. stored), Lipid 1 (4° C. stored), Lipid 3 (4° C. stored), and Lipid 5 (4° C. stored). FIG. 88D) GFP MFI in primary human T-cells after transfection with CD3-targeted LNPs based on DLn-KC2-DMA, Lipid 1, Lipid 3, and Lipid 5 after freeze-thaw cycle (−80° C. storage). FIG. 88E) % Live primary human T-cells after transfection with CD3-targeted LNPs based on DLn-KC2-DMA, Lipid 1, Lipid 3, and Lipid 5 after freeze-thaw cycle (−80° C. storage).

FIG. 89A-FIG. 89C depicts transfection of human T-cells with CD3-targeted LNPs based on Lipids 1 and 8 pre- and post freeze-thaw. FIG. 89A) % GFP+ primary human T-cells after transfection with αCD3 (SP34) targeted LNPs based on DLn-KC2-DMA, Lipid 1 (4° C. stored), Lipid 8 (4° C. stored), and Lipid 8 (−80° C. stored). FIG. 89B) GFP MFI in primary human T-cells after transfection with αCD3 (SP34) targeted LNPs based on DLn-KC2-DMA, Lipid 1 (4° C. stored), Lipid 8 (4° C. stored), and Lipid 8 (−80° C. stored). FIG. 89C) % Live primary human T-cells after transfection with αCD3 (SP34) targeted LNPs based on DLn-KC2-DMA, Lipid 1 (4° C. stored), Lipid 8 (4° C. stored), and Lipid 8 (−80° C. stored).

FIG. 90A-FIG. 90D depicts transfection of human T-cells with CD3-targeted LNPs based on Lipids 8, 9, and 10 pre- and post freeze-thaw. FIG. 90A) % GFP+ primary human T-cells after transfection with αCD3 (SP34) targeted LNPs based on DLn-KC2-DMA (−80° C. stored), Lipid 8 (4° C. stored), Lipid 9 (4° C. stored), and Lipid 10 (4° C. stored). FIG. 90B) % GFP+ primary human T-cells after transfection with αCD3 (SP34) targeted LNPs based on DLn-KC2-DMA (−80° C. stored), Lipid 8 (−80° C. stored), and Lipid 10 (−80° C. stored). FIG. 90C) GFP MFI in primary human T-cells after transfection with αCD3 (SP34) targeted LNPs based on DLn-KC2-DMA (−80° C. stored), Lipid 8 (4° C. stored), Lipid 9 (4° C. stored), and Lipid 10 (4° C. stored). FIG. 90D) GFP MFI in primary human T-cells after transfection with αCD3 (SP34) targeted LNPs based on DLn-KC2-DMA (−80° C. stored), Lipid 8 (−80° C. stored), and Lipid 10 (−80° C. stored).

FIG. 91A-FIG. 91E depicts transfection of human T-cells with CD3-targeted LNPs based on Lipids 3, 4, 9, and 15 pre- and post freeze-thaw. FIG. 91A) % GFP+ primary human T-cells after transfection with αCD3 (SP34) targeted LNPs based on DLn-KC2-DMA (−80° C. stored), Lipid 3 (4° C. stored), Lipid 4 (4° C. stored), Lipid 9 (4° C. stored), and Lipid 15 (4° C. stored). FIG. 91B) % GFP+ primary human T-cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored). FIG. 91C) GFP MFI in primary human T-cells after transfection with αCD3 (SP34) targeted LNPs based on DLn-KC2-DMA (−80° C. stored), Lipid 3 (4° C. stored), Lipid 4 (4° C. stored), Lipid 9 (4° C. stored), and Lipid 15 (4° C. stored). FIG. 91D) GFP MFI in primary human T-cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored). FIG. 91E) % Live primary human T-cells after transfection with αCD3 (SP34) targeted LNPs based on DLn-KC2-DMA (−80° C. stored), Lipid 3 (−80° C. stored), Lipid 4 (−80° C. stored), Lipid 9 (−80° C. stored), and Lipid 15 (−80° C. stored).

FIG. 92A-FIG. 92E depicts transfection of human T-cells with CD8-targeted vs non-targeted LNPs based on Lipids 3, 4, 9, and 15. FIG. 92A) % GFP+ primary human T-cells after transfection with αCD8 (Nb8) targeted or non-targeted LNPs based on Lipid 3, Lipid, 4, Lipid 9, and Lipid 15. FIG. 92B) GFP MFI in primary human T-cells after transfection with αCD8 (Nb8) targeted or non-targeted LNPs based on Lipid 3, Lipid, 4, Lipid 9, and Lipid 15. FIG. 92C) % DiI+ primary human T-cells after transfection with αCD8 (Nb8) targeted or non-targeted LNPs based on Lipid 3, Lipid, 4, Lipid 9, and Lipid 15. FIG. 92D) DiI MFI in primary human T-cells after transfection with αCD8 (Nb8) targeted or non-targeted LNPs based on Lipid 3, Lipid, 4, Lipid 9, and Lipid 15. FIG. 92E) % Live primary human T-cells after transfection with αCD8 (Nb8) targeted or non-targeted LNPs based on Lipid 3, Lipid, 4, Lipid 9, and Lipid 15.

FIG. 93A-FIG. 93E depicts transfection of human T-cells with CD8-targeted LNPs based on Lipids 10, 15, 16, 24A, and 26 pre and post freeze-thaw. FIG. 93A) % GFP+ primary human T-cells after transfection with αCD8 (Nb8) targeted LNPs based on Lipids 10, 15, 16, 24A, and 26 and comparator lipid ALC-0315 pre and post freeze-thaw. FIG. 93B) GFP MFI in primary human T-cells after transfection with αCD8 (Nb8) targeted LNPs based on Lipids 10, 15, 16, 24A, and 26 and comparator lipid ALC-0315 pre and post freeze-thaw. FIG. 93C) % DiI+ primary human T-cells after transfection with αCD8 (Nb8) targeted LNPs based on Lipids 10, 15, 16, 24A, and 26 and comparator lipid ALC-0315 pre and post freeze-thaw. FIG. 93D) DiI MFI in primary human T-cells after transfection with αCD8 (Nb8) targeted LNPs based on Lipids 10, 15, 16, 24A, and 26 and comparator lipid ALC-0315 pre and post freeze-thaw. FIG. 93E) % Live primary human T-cells after transfection with αCD8 (Nb8) targeted LNPs based on Lipids 10, 15, 16, 24A, and 26 and comparator lipid ALC-0315 pre and post freeze-thaw.

FIG. 94A-FIG. 94F depicts αCD3-targeted LNPs induce cytokine secretion and upregulation of CD69 in human T-cells in vitro. FIG. 94A) The DiI MFI and FIG. 94B) GFP MFI of human T-cells 24 hours after incubation with varying doses of mRNA-encapsulating LNPs inserted with αCD8 (Nb8 or CD8 Fab), αCD3 (CD3 Fab), or non-binding (Fab control) targeting moieties in 3 independent human donors. Data are from technical replicates (n=3). FIG. 94C) CD69 MFI of human T-cells 24 hours after incubation with varying doses of mRNA-encapsulating LNPs inserted with αCD8 (Nb8 or CD8 Fab), αCD3 (CD3 Fab), or non-binding (Fab control) targeting moieties in 3 independent human donors. Data are from technical replicates (n=3). FIG. 94D) Histograms of CD69 values in one representative donor (Donor 1) illustrating the differences between cells treated with αCD3-targeted LNPs and αCD8-targeted LNPs. FIG. 94E) The level of DiI-LNP association over time with T-cells evaluated with the Incucyte SX5 and illustrated as the Near InfraRed (NIR) Integrated Intensity (NIRCU). Data are from biologically independent replicates (n=3). FIG. 94F) Levels of TNF-α detected in the supernatants 24 hours after incubation with varying doses of mRNA-encapsulating LNPs in 3 independent human donors. Data are from technical replicates (n=3). The displayed p-values are from a two-way ANOVA.

FIG. 95A-FIG. 95E depicts comparison of LNPs inserted with humanized variants of Nb8. FIG. 95A) The fraction of live T-cells 24 hours after incubation with 1, 0.3, and 0.06 μg/mL mRNA encapsulated in targeted LNPs inserted with humanized variants of Nb8 at different densities. Each bar represents the mean of technical duplicates (n=2). FIG. 95B) The DiI-positive fraction and FIG. 95C) DiI MFI of primary human T-cells 24 hours after incubation with 1, 0.3, and 0.06 μg/mL mRNA encapsulated in targeted LNPs inserted with humanized variants of Nb8 at different densities. Data are mean of values in n=2 replicates. FIG. 95D) The GFP-positive fraction and FIG. 95E) GFP MFI of primary human T-cells 24 hours after incubation with 1, 0.3, and 0.06 μg/mL mRNA encapsulated in targeted LNPs inserted with humanized variants of Nb8 at different densities. Data are mean of values in n=2 replicates.

FIG. 96A-FIG. 96Q depicts αCD8 ISVD-targeted LNPs mediate functional CAR T-cell reprogramming. FIG. 96A) CAR library design (left panel) and screening data from Jurkat activation assay (right panel). Data are from technical replicates (n=2). FIG. 96B) Cytotoxicity assay comparing 22-V4 containing a CD28 co-stimulatory domain to 22-V4 containing a 4-1BB co-stimulatory domain against Nalm6 cells at various effector-to-target cell ratios (E:Ts). FIG. 96C) Number of CD19 and FIG. 96D) CD22 surface receptors per cell in different cancer cell lines, quantified using Quantibrite PE beads. Data are from technical replicates (n=3). FIG. 96E) The fraction of CD19 CAR− and FIG. 96F) CD22 CAR-positive primary human T-cells and FIG. 96G) the MFI values of CD19 CAR− and FIG. 96H) CD22 CAR-expressing primary human T-cells 24 hours after incubation with humanized αCD8 VHH (Nb8-H3) targeted LNPs encapsulating mRNA encoding a CD22 (22-V4 or m971) or a CD19 (FMC63) chimeric antigen receptor (CAR) or a non-CAR payload (LNP control). Data are from biologically independent replicates (n=3). FIG. 96I-FIG. 96N) CAR T-cells were co-cultured with cancer cell lines at increasing E:Ts and the level of CAR-mediated cytotoxicity was evaluated 48 hours later. Data are from biologically independent replicates (n=3). The displayed p-values are from two-tailed paired t-tests comparing 22-V4 to m971. FIG. 96O) Levels of IFN-γ, FIG. 96P) Granzyme B, and FIG. 96Q) IL-10 detected in the supernatants 48 hours after LNP treatment and co-culture. Data are from biologically independent replicates (n=3).

FIG. 97A-FIG. 97L depicts transfection specificity and B-cell aplasia in PBMC assay. FIG. 97A) t-distributed stochastic neighbor embedding (t-SNE) analysis was performed on 22-V-treated (left panel) and PBS-treated (right panel) PBMCs and grouped using FlowSOM in OMIQ to visualize various cell subsets. FIG. 97B) Cell subsets were defined based on the expression of CD3, CD4, CD8, CD14, CD20, and CD159a surface markers. Monocytes were defined as CD14+, B-cells were defined as CD20+, CD8 T-cells were defined as CD3+CD4-CD8+, CD4 T-cells were defined as CD3+CD4+CD8−, DP T-cells were defined as CD3+CD4+CD8+, DN T-cells were defined as CD3+CD4−CD8−CD159a−, NK cells were defined as CD3−CD159a+, NKT cells were defined as CD3+CD159a+CD8−, and CD8+ NKT cells were defined as CD3+CD159a+CD8+. Heatmap overlays showing the relative expression of FIG. 97C) CD20, FIG. 97D) CD3, FIG. 97E) CD4, FIG. 97F) CD8α, FIG. 97G) CD159a, FIG. 97H) CD14, FIG. 97I) CAR, FIG. 97J) CD45RA, FIG. 97K) CCR7, FIG. 97L) and CD69. Data shown are from one representative donor (Donor 3).

FIG. 98A-FIG. 98F depicts cytotoxicity assessment with relevant cell lines. FIG. 98A-FIG. 98F) CAR T-cells were co-cultured with cancer cell lines at increasing E:Ts and the level of CAR-mediated cytotoxicity was evaluated 48 hours later. Data are from technical replicates (n=3).

FIG. 99A-FIG. 99E depicts titrating dsRNA to identify immunogenicity threshold. FIG. 99A) IFN-α2a, FIG. 99B) IFN-γ, FIG. 99C) RANTES, FIG. 99D) IL-10, and FIG. 99E) IL-6 levels were evaluated in the MIMIC® assay in groups treated with CAR-LNPs containing different levels of dsRNA and compared to CAR-LNPs containing RP-HPLC-purified mRNA. Data are from biologically independent replicates (n=6). The displayed p-values are from one-way ANOVA.

FIG. 100A-FIG. 100C depicts Flow Cytometry gating strategy. Flow Cytometry gating strategy for FIG. 100A) ex vivo whole blood assay, FIG. 100B) in vivo CAR reprogramming, and FIG. 100C) myeloid cell subset identification in the liver.

FIG. 101A-FIG. 101C: CryoEM structure of hCD8αβ-A044300805_v8-Fab38 complex at 3.27 Å; (FIG. 101A) CryoEM Map; (FIG. 101B) Model surface representation; (FIG. 101C) Local refinement: C50-C104 (C50-C100 according to Kabat numbering) clips together CDR2 and CDR3. Extended CDR3 amino acids 105-120 (100a-101) are flexible.

FIG. 102A-FIG. 102D: Interface analysis of human CD8αβ in complex with ISVD A044300805_v8 (FIG. 102A). FIG. 102B shows the interface area; FIG. 102C shows the surface charge distribution; and FIG. 102D shows surface hydrophobicity.

FIG. 103A-FIG. 103E: Detailed views of interactions between ISVD A044300805_v8 and CD8alpha. (FIG. 103A) List of interacting residues of epitope on CD8alpha located at <4.0 Å distance from ISVD A044300805_v8. (FIG. 103B) List of interacting residues of paratope of ISVD A044300805_v8 located at <4.00 Å distance from the CD8. (FIG. 103C) Key interacting residues at the ISVD A044300805_v8-CD8alpha interface: salt bridges are indicated as red dash lines and hydrogen bonds are indicated as blue dash lines. (FIG. 103D) ISVD A044300805_v8-CD8alpha interface: salt bridge between the CD8alpha and ISVD A044300805_v8 (dotted line). (FIG. 103E) ISVD A044300805_v8-CD8alpha interface: hydrogen bounds between CD8alpha and ISVD A044300805_v8 (dotted lines).

FIG. 104A: The binding site of ISVD A044300805_v8 interaction to CD8alpha partially overlaps with the interaction site between Class I MHC-β2-microglobulin and CD8alpha.

FIG. 104B: Curved architecture formed by the extended CDR3 recapitulates the contour of MHC molecules that naturally engage CD8 on the top, suggesting a maximization of optimal recognition surface by ISVD A044300805_v8.

### DETAILED DESCRIPTION-LIPID NANOPARTICLES

The following description sets forth exemplary methods, parameters and the like. It should be recognized, however, that such description is not intended as a limitation on the scope of the present disclosure but is instead provided as a description of exemplary embodiments.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH2CH2-.

The term “haloalkyl” refers to an alkyl group that is substituted with at least one halogen. For example, —CH2F, —CHF2, —CF3, —CH2CF3, —CF2CF3, and the like.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl (allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “oxo” is art-recognized and refers to a “═O” substituent. For example, a cyclopentane substituted with an oxo group is cyclopentanone.

The term “morpholinyl” refers to a substituent having the structure of:

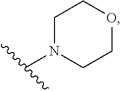 [SMILES: *N1CCOCC1]

which is optionally substituted.

The term “piperidinyl” refers to a substituent having a structure of:

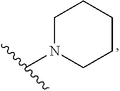 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, or —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C3-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₁₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula-N(R¹⁰) (R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH2)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “ [symbol] ” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The present disclosure also embraces isotopically labeled compounds of the present disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in, e.g., the Examples herein by substituting an isotopically labeled reagent for a non-isotopically labeled reagent.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an affinity ligand, in particular, an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the present disclosure and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, 2-maleate, methanesulfonate, naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Nat, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl) carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCI), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a VHH (including a humanized VHH), a VH (including a camelized VH, a human VH, a camelized human VH, and a dAb) or a VL.

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired immune cell type that receives the delivered nucleic acid (e.g., on-target delivery), and % of an undesired immune cell type that is not meant to be the destination of the delivery, but receives the delivered nucleic acid (e.g., off-target delivery). For example, the specificity is higher when more desired immune cells receive the delivered nucleic acid, while less undesired immune cells receive the delivered nucleic acid. Specificity of the targeted delivery by an LNP can also be defined by the ratio of amount of nucleic acid being delivered to the desired immune cells (e.g., on-target delivery) and amount of nucleic acid being delivered to the undesired immune cells (e.g., off-target delivery). Specificity of the delivery can be determined using any suitable method. As a non-limiting example, expression level of the nucleic acid in the desired immune cell type can be measured and compared to that of a different immune cell type that is not meant to be the destination of the delivery.

As used herein, in some embodiments, a reference LNP is an LNP that does not have the immune cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the present disclosure unless claimed. No language in the specification should be construed as indicating any non-claimed element as essential to the practice of the present invention.

As used herein, the term “pseudouridine” refers to the natural product which is a C-glycosyl pyrimidine that consists of uracil having a beta-D-ribofuranosyl residue attached at position 5 (i.e., 5-(beta-D-Ribofuranosyl) uracil). In some embodiments, the term refers to m′acphi/(1-methyl-3-(3-amino-3-carboxypropyl) pseudouridine. In another embodiment, the term refers to mlvP (1-methylpseudouridine). In another embodiment, the term refers to *|/m (2′-O-methylpseudouridine. In another embodiment, the term refers to m5D (5-methyldihydrouridine). In another embodiment, the term refers to m3\|/(3-methylpseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

As used herein, the terms “lipid-PEG” and “PEG-lipid” are interchangeable, referring to PEG derivatives in which PEG is attached with a lipid moiety. PEG-lipid can be used to improve circulation times for liposome encapsulated (LNP) drugs and reduce non-specific uptakes. If the lipid is a phospholipid, the molecule can be referred as “phospholipid-PEG” or “PEG-phospholipid”. Any suitable chemistry may be used to conjugate a polypeptide to the PEG of the PEG-lipid, see Parhiz et al., Journal of Controlled Release 291:106-115, 2018; Kolb et al., Angewandte Chemie International Edition 40 (11): 2004-2021, 2001; and Evans, Australian Journal of Chemistry 60 (6): 384-395, 2007. For example, lipid-PEG-maleimide, lipid-PEG-cysteine, lipid-PEG-alkyne, PEG-dibenzocyclooctyne (DBCO), lipid-PEG-bromo maleimide, lipid-PEG-alkylnoic amide, PEG-alkynoic imide, and lipid-PEG-azide can be used to produce a Lipd-PEG-polypeptide conjugate.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In some embodiments, when a domain, antibody, or sequence is derived from another domain, antibody, or sequence, then the domain, antibody, or sequence is the same as the other domain, antibody, or sequence. In some embodiments, when a domain, antibody, or sequence is derived from another domain, antibody, or sequence, the domain, antibody, or sequence has at least 80%, at least 85%, at least 90%, at least 95%, at least 96%, at least 97%, at least 98%, or at least 99% identity with the other domain, antibody, or sequence. In some embodiments, when a domain, antibody, or sequence is derived from another domain, antibody, or sequence, the domain, antibody, or sequence has 3, 2, or 1 amino acid difference.

The terms “epitope” and “antigenic determinant”, which can be used interchangeably, refer to the part of a macromolecule, such as a polypeptide or protein that is recognized by antigen-binding molecules, such as immunoglobulins, conventional antibodies, or immunoglobulin single variable domains, and more particularly by the antigen-binding site of said molecules. Epitopes define the minimum binding site for an immunoglobulin, and thus represent the target of specificity of an immunoglobulin. The part of an antigen-binding molecule (such as an immunoglobulin, a conventional antibody, an immunoglobulin single variable domain) that recognizes the epitope is called a “paratope”.

Protein-protein interactions (PPIs) are highly specific physical contacts, stable or transient in time, between two or more proteins. PPIs between polypeptides can be determined experimentally i.e., affinity chromatography, tandem affinity purification (TAP), co-immunoprecipitation, Yeast two-hybrid screening X-ray crystallography, Cryogenic electron microscopy (cryo-EM), nuclear magnetic resonance, spectroscopy, Hydrogen/Deuterium eXchange Mass Spectrometry (HDX-MS), and protein arrays etc. or via in silico technique.

The term “interact with” as used herein in the context of at least two polypeptides forming a complex (e.g. anti-CD8 ISVD and CD8) means that at least one (amino acid) residue of one polypeptide is in close proximity to at least one (amino acid) residue of the other polypeptide. The distance between two (amino acid) residues which are located within distinct polypeptides may be determined using methods known is the art. For example, the skilled person knows that structural information allowing to determine the distance between two (amino acids) residues may be obtained using standard methods such as X-ray crystallography, Cryogenic electron microscopy (cryo-EM), nuclear magnetic resonance, and subsequent molecular modelling. The skilled person is also aware that two (amino acid) residues are in close proximity and therefore interact with each other if the (shortest) distance between the two (amino acid) residues is less than 10 Å, less than 8 Å, less than 6 Å, less than 5 Å, less than 4 Å, less than 3 Å, less than 2 Å, preferably less than 4 Å, more preferably less than 4 Å and more than 2 Å.

The term “interaction site” as used herein refers to the area within a complex comprising at least two polypeptides which is formed by the (amino acid) residue(s) within each of the respective polypeptides that interact with each other as defined herein. For example, an ISVD specifically binding to CD8alpha comprises at least one amino acid residue that interacts with at least one (amino acid) residue on CD8alpha. In this respect, the at least one amino acid residue of the anti-CD8 ISVD and the at least one (amino acid) residue on CD8alpha interacting with each other also form an interaction site. The skilled person is aware that interaction sites may for example be defined by reference to a specific amino acid residue of the polypeptide (e.g. N76 of CD8alpha).

Molecular interactions can have a different strength, bond length (Å), interaction area (Å2), depending on the type of amino acid residues that make up the interaction. Strong molecular interactions include hydrogen bonds (or H-bonds), that are electrostatic forces of attraction between a hydrogen (H) atom which is covalently bonded to a more electronegative donor atom or group, and another electronegative atom bearing a lone pair of electrons, the hydrogen bond acceptor. Such an interacting system is generally denoted “donor-H ⋅ ⋅ ⋅ acceptor”, where the solid line denotes a polar covalent bond, and the dotted or dashed line indicates the hydrogen bond. The most frequent donor and acceptor atoms are the period 2 elements: nitrogen (N), oxygen (O), and fluorine (F). The hydrogen bonds have a length around 1.5-2.5 Å and an interaction area around 10-50 Å². Typical amino acid residues that form a H-bond are Lys, Arg, Asn, Gln, Ser and Thr (donors), and Asp, Glu, Asn, Gln, Ser and Thr (acceptors).

Other strong molecular interactions include salt bridges, that are defined as electrostatic interactions between two oppositely charged groups: the anionic carboxylate of either glutamate (E) or aspartate (D), and, the cationic ammonium from either arginine (R) or lysine (K). The salt bridges have a length around 2.8-4.0 Å and an interaction area around 10-100 Å². Typical amino acid residues that form a salt bridge are Lys, Arg and His (positive), and Asp and Glu (negative).

Weaker interactions include hydrophobic interactions, that are the non-covalent force where nonpolar species tend to cluster in water in order to decrease the overall interfacial area between the hydrophobic species and water. Hydrophobic interactions have a length around 3.5-4.5 Å and an interaction area around 100-300 Å². Typical amino acid residues that form hydrophobic interactions are Ala, Val, Leu, Ile, Met, Phe, Trp.

Hydrogen bonds and salt bridges indicate specificity and stability, while hydrophobic contacts support affinity.

Van der Waals forces include attraction and repulsions between atoms, molecules, as well as other (weaker) intermolecular forces. They differ from covalent and ionic bonding in that they are caused by correlations in the fluctuating polarizations of nearby particles. The force results from a transient shift in electron density. Specifically, the electron density may temporarily shift to be greater on one side of the nucleus. This shift generates a transient charge which a nearby atom can be attracted to or repelled by. Van der Waals forces have a length around 3.5-4.5 Å and an interaction area around 50-200 Å^{2.}

Molecular modelling of the interaction site can be performed by tools known in the art, such as e.g., PDBe-PISA. PDBe-PISA is an interactive tool for the exploration of macromolecular interfaces based on cryo-EM or Xray structures (https://www.ebi.ac.uk/pdbe/pisa/). PDBe-PISA represents a systematic approach to automatic identification of probable quaternary structures, based on physical-chemical models of macromolecular interactions and chemical thermodynamics. Amongst others, PDBe-PISA calculates buried surface area (BSA) according to cryoEM/Xray structures, which is the portion of molecular surfaces no longer exposed to solvent upon complex formation. Core interacting residues are deeply buried, have a high buried surface area (BSA) and low solvent exposure (ASA, Accessible Surface Area). Interface stability can be assessed using solvation free energy gain upon binding (ΔiG), as calculated by PISA. Negative ΔiG values are indicative of hydrophobic interfaces and favorable complex formation. (E. Krissinel and K. Henrick (2005). Detection of Protein Assemblies in Crystals. In: M. R. Berthold et. al. (Eds.): CompLife 2005, LNBI 3695, pp. 163-174. Springer-Verlag Berlin Heidelberg).

The affinity is a measure for the binding strength between a moiety and a binding site on the target molecule: the lower the value of the K_{D}, the stronger the binding strength between a target molecule and a targeting moiety. Typically, binding units used in the present technology, such as ISVDs, will bind to their targets with a dissociation constant (K_{D}) of 10⁻⁵ to 10⁻¹² moles/liter or less, 10⁻⁷ to 10⁻¹² moles/liter or less, or 10⁻⁸ to 10⁻¹² moles/liter (i.e. with an association constant (K_{A}) of 10⁵ to 10¹² liter/moles or more, 10⁷ to 10¹² liter/moles or more, or 10⁸ to 10¹² liter/moles). Any K_{D} value greater than 10⁻⁴ mol/liter (or any K_{A} value lower than 104 liters/mol) is generally considered to indicate non-specific binding. The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 μM) or less.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned further herein.

The affinity of a molecular interaction between two molecules can be measured via different techniques known per se, such as the well-known surface plasmon resonance (SPR) biosensor technique (see for example Ober et al. 2001, Intern. Immunology 13:1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kon, koff measurements and hence KD (or KA) values. This can for example be performed using the well-known BIAcore® system (BIAcore International AB, a GE Healthcare company, Uppsala, Sweden and Piscataway, NJ) or the ProteOn™ XPR36 Protein Interaction Array System (Bio-rad Laboratories, Inc). For further descriptions, see Jonsson et al. 1993 (Ann. Biol. Clin. 51:19-26), Jonsson et al. 1991 (Biotechniques 11:620-627), Johnsson et al. 1995 (J. Mol. Recognit. 8:125-131), and Johnnson et al. 1991 (Anal. Biochem. 198:268-277).

Another well-known biosensor technique to determine affinities of biomolecular interactions is bio-layer interferometry (BLI) (see for example Abdiche et al. 2008, Anal. Biochem. 377:209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328:35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5:1765-74).

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (KA), by means of the relationship [K_{D}=1/K_{A}].

The terms “block”, “antagonize”, “compete”, “competing” and “-competition” are used interchangeably herein to mean the ability of an immunoglobulin, antibody, immunoglobulin single variable domain, polypeptide or other binding agent to interfere with the binding of another protein, polypeptides, ligand or binding agent to a given target. The extent to which an immunoglobulin, antibody, immunoglobulin single variable domain, polypeptide or other binding agent is able to interfere with the binding of another ligand to the target, and therefore whether it can be said to “block”, can be determined using competition binding assays. Particularly suitable quantitative competitive blocking assays are described in the Examples and include e.g. a fluorescence-activated cell sorting (FACS) binding assay with CD8 expressed on cells. The extent of blocking can be measured by the (reduced) channel fluorescence.

Other methods for determining whether an immunoglobulin, antibody, immunoglobulin single variable domain, polypeptide or other binding agent directed against a target blocks, is capable of blocking, competitively binds or is competitive as defined herein are described e.g. in Xiao-Chi Jia et al. (Journal of Immunological Methods 288:91-98, 2004), Miller et al. (Journal of Immunological Methods 365:118-125, 2011).

As used herein, the term “potency” is a measure of the biological activity of an agent, such as an ISVD or polypeptide. Potency of an agent can be determined by any suitable method known in the art, such as for instance as described in the experimental section. Cell culture-based potency assays are often the preferred format for determining biological activity since they measure the physiological response elicited by the agent and can generate results within a relatively short period of time. Various types of cell-based assays, can be used to determine potency, such as e.g. binding of the ISVD to CD8⁺ T cells (as further described in the Example section).

### II. Immunoglobulin Single Variable Domain

The present disclosure provides immune cell targeting LNPs comprising an immune cell targeting group. In some embodiments, the immune cell targeting group of the LNPs as described herein comprise an immunoglobulin single variable domain, such as a VHH (including a humanized VHH), a VH (including a camelized VH, a human VH, a camelized human VH, and a dAb) or a VL.

The term “immunoglobulin single variable domain” (ISVD), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e., a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a VH-VL pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single VH, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof; or a heavy chain variable domain sequence (e.g., a V_{H}-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. In one embodiment, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISVD (as defined herein and including but not limited to a V_{HH}); other single variable domains, or any suitable fragment of any one thereof.

In particular, the immunoglobulin single variable domain may be a Nanobody® ISVD (such as a V_{HH}, including a humanized V_{HH} or camelized V_{H}) or a suitable fragment thereof. [Note: Nanobody® and Nanobodies® is a registered trademark of Ablynx N.V.].

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993 (Nature 363:446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'S, reference is made to the review article by Muyldermans 2001 (Reviews in Molecular Biotechnology 74:277-302).

For the term “dAb's” and “domain antibody”, reference is for example made to Ward et al. 1989 (Nature 341:544), to Holt et al. 2003 (Trends Biotechnol. 21:484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries, e.g., by phage display.

The generation of immunoglobulin sequences, such as VHHs, has been described extensively in various publications, among which WO 1994/04678, Hamers-Casterman et al. 1993 (Nature 363:446-448) and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. 1989 (Nature 341:544), WO 1994/04678, and Davis and Riechmann (1994, Febs Lett., 339:285-290; and 1996, Prot. Eng., 9:531-537) can be used herein. Moreover, the ISVDs are fused forming a multivalent and/or multispecific construct (for multivalent and multispecific polypeptides containing one or more VHH domains and their preparation, reference is also made to Conrath et al. 2001 (J. Biol. Chem., Vol. 276, 10. 7346-7350) as well as to for example WO 1996/34103 and WO 1999/23221).

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies and Riechman 1994, FEBS 339:285; 1995, Biotechnol. 13:475; 1996, Prot. Eng. 9:531; and Riechman 1999, J. Immunol. Methods 231:25). Such “camelizing” substitutions are inserted at amino acid positions that form and/or are present at the V_{H}-V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

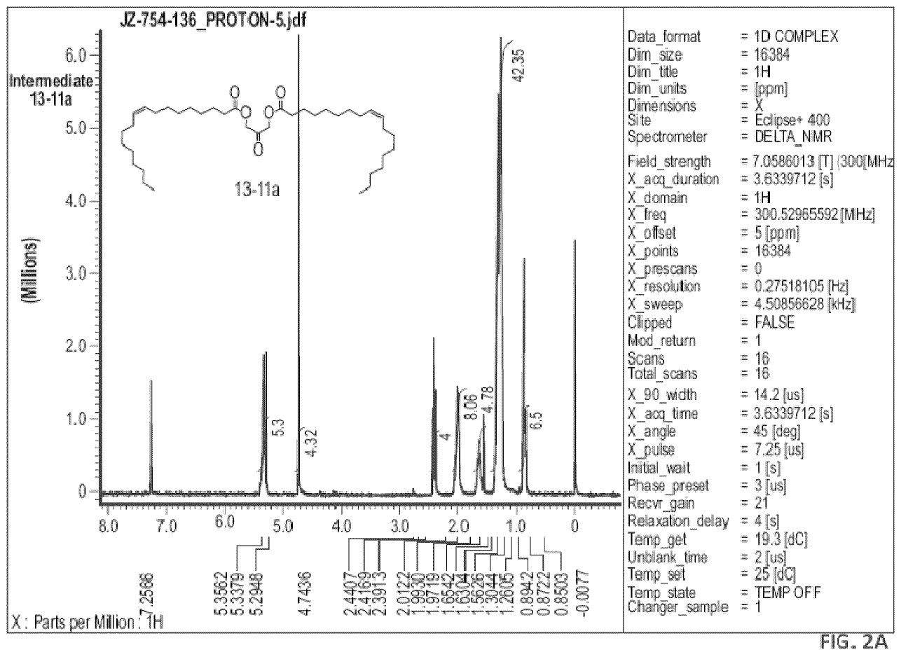

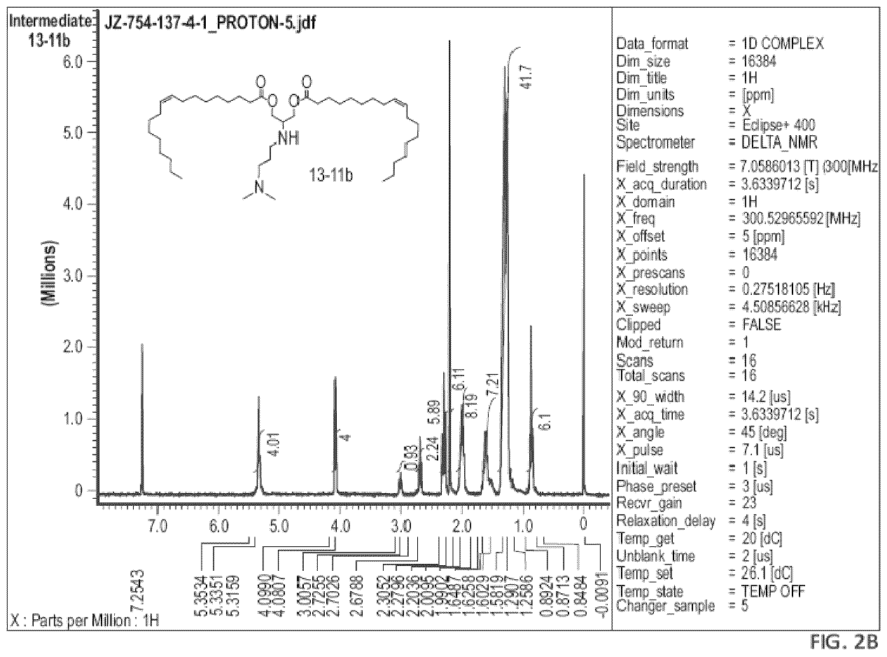

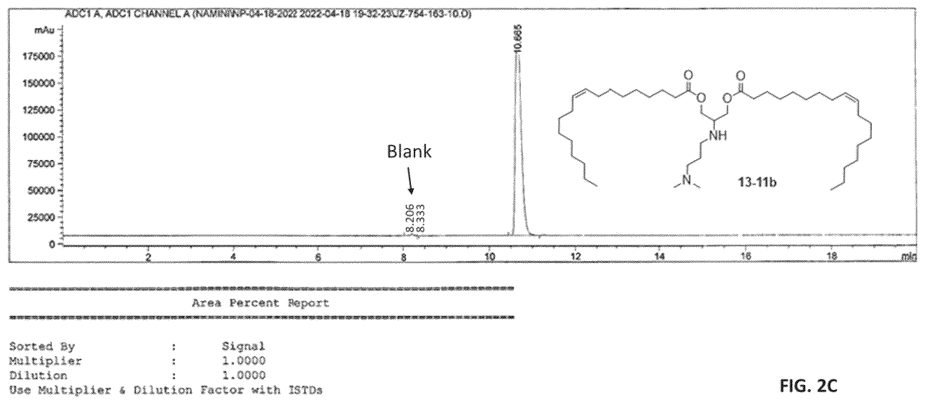

The amino acid residues of an ISVD can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein. Also see The Kabat Numbering Scheme by Prof. Andrew C. R. Martine's Group in bioinf.org.uk, and Protein Sequence and structure Analysis of Antibody Variable domains by Prof. Andrew C. R. Martin in Antibody Engineering Vol. 2, Chapter 3, DOI 10.1007/978-3-642-01147-4_3.

In the present application, unless indicated otherwise, CDR sequences were determined according to the AbM definition as described in Kontermann and Dübel (Eds. 2010, Antibody Engineering, vol 2, Springer Verlag Heidelberg Berlin, Martin, Chapter 3, pp. 33-51). According to this method, FR1 of an ISVD comprises the amino acid residues at positions 1-25, CDR1 of an ISVD comprises the amino acid residues at positions 26-35, FR2 of an ISVD comprises the amino acids at positions 36-49, CDR2 of an ISVD comprises the amino acid residues at positions 50-58, FR3 of an ISVD comprises the amino acid residues at positions 59-94, CDR3 of an ISVD comprises the amino acid residues at positions 95-102, and FR4 of an ISVD comprises the amino acid residues at positions 103-113.

Determination of CDR regions may also be done according to different methods. In the CDR definition according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence described herein may contain one or more of hallmark residues (as defined herein), such that the ISVD sequence is a Nanobody® ISVD, such as, e.g., a V_{HH}, including a humanized V_{HH} or camelized V_{H}. Non-limiting examples of (suitable combinations of) such framework sequences will become clear from the further disclosure herein.

The total number of amino acid residues in a Vu domain and a V_{HH} domain will usually be in the range of from 110 to 120, often between 112 and 115. It should however be noted that smaller and longer sequences may also be suitable for the purposes described herein.

However, it should be noted that the ISVDs described herein is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVDs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISVD can be defined as an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISVD can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISVD can be an immunoglobulin sequence with the (general) structure FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVDs

| Position | Human V_{H}3 | Hallmark Residues |
|---|---|---|
| 11 | L, V; | L, S, V, M, W, F, T, Q, E, A, R, G, K, Y, N, P, I; |
| | redominantly L | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, |
| | | I; |
| | | preferably G⁽²⁾, E⁽³⁾ or Q⁽³⁾; most preferably G⁽²⁾ or Q⁽³⁾ |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W. Y | F⁽¹⁾, L⁽¹⁾ or W⁽²⁾ G, I, S, A, V, M, R, Y, E, P, T, C, H, |
| | | K, Q, N, D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; | P⁽⁵⁾, S, H, L, A, V, I, T, F, D, R, Y, N, Q, G, E; |
| | predominantly A | preferably P |
| 103 | W | W⁽⁴⁾, R⁽⁶⁾, G, S, K, A, M, Y, L, F, T, N, V, Q, P⁽⁶⁾, E, |
| | | C; preferably W |
| 104 | G | G, A, S, T, D, P, N, E, C, L; preferably G |
| 108 | L, M or T; | Q, L⁽⁷⁾, R, P, E, K, S, T, M, A, H; preferably Q or L⁽⁷⁾ |
| | predominantly L | |

Notes:
⁽¹⁾In particular, but not exclusively, in combination with KERE (SEQ ID NO: 372) or KQRE (SEQ ID NO: 373) at positions 43-46.
⁽²⁾Usually as GLEW (SEQ ID NO: 374) at positions 44-47.
⁽³⁾Usually as KERE (SEQ ID NO: 372) or KQRE (SEQ ID NO: 373) at positions 43-46, e.g., as KEREL (SEQ ID NO: 375), KEREF (SEQ ID NO: 376), KQREL (SEQ ID NO: 377), KQREF (SEQ ID NO: 378), KEREG (SEQ ID NO: 379), KQREW (SEQ ID NO: 380) or KQREG (SEQ ID NO: 381) at positions 43-47. Alternatively, also sequences such as TERE (SEQ ID NO: 382) (for example TEREL (SEQ ID NO: 383)), TQRE (SEQ ID NO: 384) (for example TQREL (SEQ ID NO: 385)), KECE (SEQ ID NO: 386) (for example KECEL (SEQ ID NO: 387) or KECER (SEQ ID NO: 388)), KQCE (SEQ ID NO: 389) (for example KQCEL (SEQ ID NO: 390)), RERE (SEQ ID NO: 391) (for example REREG (SEQ ID NO: 392)), RQRE (SEQ ID NO: 393) (for example RQREL (SEQ ID NO: 394), RQREF (SEQ ID NO: 395) or RQREW (SEQ ID NO: 396)), QERE (SEQ ID NO: 397) (for example QEREG (SEQ ID NO: 398)), QQRE (SEQ ID NO: 399), (for example QQREW (SEQ ID NO: 400), QQREL (SEQ ID NO: 401) or QQREF (SEQ ID NO: 402)), KGRE (SEQ ID NO: 403) (for example KGREG (SEQ ID NO: 404)), KDRE (SEQ ID NO: 405) (for example KDREV (SEQ ID NO: 406)) are possible. Some other possible, but less preferred sequences include for example DECKL (SEQ ID NO: 407) and NVCEL (SEQ ID NO: 408).
⁽⁴⁾With both GLEW (SEQ ID NO: 374) at positions 44-47 and KERE (SEQ ID NO: 372) or KQRE (SEQ ID NO: 373) at positions 43-46.
⁽⁵⁾Often as KP or EP at positions 83-84 of naturally occurring V_{HH} domains.
⁽⁶⁾In particular, but not exclusively, in combination with GLEW (SEQ ID NO: 374) at positions 44-47.
⁽⁷⁾With the proviso that when positions 44-47 are GLEW (SEQ ID NO: 374), position 108 is always Q in (non-humanized) V_{HH} sequences that also contain a W at 103.
⁽⁸⁾The GLEW group also contains GLEW-like sequences at positions 44-47, such as for example GVEW (SEQ ID NO: 409), EPEW (SEQ ID NO: 410), GLER (SEQ ID NO: 411), DQEW (SEQ ID NO: 412), DLEW (SEQ ID NO: 413), GIEW (SEQ ID NO: 414), ELEW (SEQ ID NO: 415), GPEW (SEQ ID NO: 416), EWLP (SEQ ID NO: 417), GPER (SEQ ID NO: 418), GLER (SEQ ID NO: 411) and ELEW (SEQ ID NO: 415).

In one embodiment, the immunoglobulin single variable domain has certain amino acid substitutions in the framework regions effective in preventing or reducing binding by so-called “pre-existing antibodies” to the polypeptides. To this end, in one embodiment, the polypeptide comprises a valine (V) at amino acid position 11 and a leucine (L) at amino acid position 89 (according to Kabat numbering) in at least one ISVD. In one embodiment, the polypeptide comprises a valine (V) at amino acid position 11 and a leucine (L) at amino acid position 89 (according to Kabat numbering) in each ISVD. Accordingly, part of the present disclosure are also ISVDs and polypeptides as described above that have been sequence optimized with a valine (V) at amino acid position 11 and a leucine (L) at amino acid position 89 (according to Kabat numbering) in at least one ISVD, such as in all ISVDs. Examples of ISVDs and polypeptides that comprises a valine (V) at amino acid position 11 and a leucine (L) at amino acid position 89 (according to Kabat numbering) in at least one ISVD are depicted in Table C-2 (SEQ ID NOs: 161-169, 171-179 and 28-36 and 44).

In one embodiment, the ISVD or polypeptide has a C-terminal end of the sequence VTVSS(X)n (SEQ ID NO: 353), in which n is 1 to 10, preferably 1 to 5, such as 1, 2, 3, 4 or 5, and in which each X is an amino acid residue that is independently chosen. In one embodiment, the polypeptide comprises such an ISVD at its C-terminal end. In one embodiment, n is 1 or 2, such as 1. In one embodiment, X is a naturally occurring amino acid. In one embodiment, X is chosen from the group consisting of alanine (A), glycine (G), valine (V), leucine (L) or isoleucine (I).

In another embodiment the polypeptide comprises a lysine (K) or glutamine (Q) at position 110 (according to Kabat numbering) in at least one ISVD. In another embodiment, the ISVD comprises a lysine (K) or glutamine (Q) at position 112 (according to Kabat numbering) in at least one ISVD. In these embodiments, the C-terminus of the ISVD is VKVSS (SEQ ID NO: 354), VQVSS (SEQ ID NO: 355), VTVKS (SEQ ID NO: 356), VTVQS (SEQ ID NO: 357), VKVKS (SEQ ID NO: 358), VKVQS (SEQ ID NO: 359), VQVKS (SEQ ID NO: 360), or VQVQS (SEQ ID NO: 361) such that after addition of a single alanine the C-terminus of the polypeptide for example comprises the sequence VTVSSA (SEQ ID NO: 362), VKVSSA (SEQ ID NO: 363), VQVSSA (SEQ ID NO: 364), VTVKSA (SEQ ID NO: 365), VTVQSA (SEQ ID NO: 366), VKVKSA (SEQ ID NO: 367), VKVQSA (SEQ ID NO: 368), VQVKSA (SEQ ID NO: 369), or VQVQSA (SEQ ID NO: 370). In one embodiment, the polypeptide comprises a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD, and comprises an extension of 1 to 5 (naturally occurring) amino acids (as defined above), such as a single alanine (A) extension, at the C-terminus of the C-terminal ISVD, such that the C-terminus of the polypeptide for example comprises the sequence VTVSSA (SEQ ID NO: 362), VKVSSA (SEQ ID NO: 363) or VQVSSA (SEQ ID NO: 364). See e.g. WO2012/175741 and WO2015/173325 for further information in this regard.

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally contain one or more further amino acids that can serve as a binding unit, so as to provide a monovalent, multivalent or multispecific polypeptide of the present disclosure, respectively (for multivalent and multispecific polypeptides containing one or more VHH domains and their preparation, reference is also made to Conrath et al. 2001 (J. Biol. Chem. 276:7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVDs in a polypeptide. In one embodiment, the polypeptide is “bivalent”, i.e., comprises or consists of two ISVDs. In one embodiment, the polypeptide is “trivalent”, i.e., comprises or consists of three ISVDs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVDs, respectively. In one embodiment the multivalent ISVD polypeptide is trivalent. In another embodiment the multivalent ISVD polypeptide is tetravalent. In still another embodiment, the multivalent ISVD polypeptide is pentavalent.

In one embodiment, the multivalent ISVD polypeptide can also be multispecific. The term “multispecific” refers to binding to multiple different target molecules (also referred to as antigens). The multivalent ISVD polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVDs, wherein two ISVDs bind to a first target and one ISVD binds to a second target different from the first target. In another example, the polypeptide may be trispecific-tetravalent, such as a polypeptide comprising or consisting of four ISVDs, wherein one ISVD binds to a first target, two ISVDs bind to a second target different from the first target and one ISVD binds to a third target different from the first and the second target. In still another example, the polypeptide may be trispecific-pentavalent, such as a polypeptide comprising or consisting of five ISVDs, wherein two ISVDs bind to a first target, two ISVDs bind to a second target different from the first target and one ISVD binds to a third target different from the first and the second target.

In one embodiment, the multivalent ISVD polypeptide can also be multiparatopic. The term “multiparatopic” refers to binding to multiple different epitopes on the same target molecules (also referred to as antigens). The multivalent ISVD polypeptide can thus be “biparatopic”, “triparatopic”, etc., i.e., can bind to two, three, etc., different epitopes on the same target molecules, respectively.

In another aspect, the polypeptide of the present disclosure that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb” s, amino acids that are suitable for use as a dAb, VHHs (including humanized VHHs), VHs (including human VHs, camelized VHs and camelized human VHs), and VLs.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding by so-called “pre-existing antibodies” to the polypeptides. For this purpose, the polypeptides and constructs may contain a C-terminal extension (X)n (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n, in which n is 1 to 5, such as 1, 2, 3, 4 or 5, and in which X is a naturally occurring amino acid, preferably no cysteine.

In one embodiment, the polypeptide may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t½beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG. In one embodiment, said one or more other groups, residues, moieties or binding units that provide the polypeptide with increased half-life is a binding unit that can bind to human serum albumin. In one embodiment, the binding unit is an ISVD.

For example, WO 2004/041865 and WO 2006/122787 describes ISVDs binding to serum albumin (and in particular against human serum albumin) that can be linked to other proteins (such as one or more other ISVDs binding to a desired target) in order to increase the half-life of said protein. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of therapeutic proteins and polypeptide and other therapeutic entities or moieties. Moreover, WO 2012/175400 describes a further improved version of Alb-1, called Alb-23.

In one embodiment, the polypeptide comprises a serum albumin binding moiety selected from Alb-1, Alb-3, Alb-4, Alb-5, Alb-6, Alb-7, Alb-8, Alb-9, Alb-10 (WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO2015/173325, WO2017/080850, WO2017/085172, WO2018/104444, WO2018/134235, and WO2018/134234. Some serum albumin binders are also shown in Table B. In one embodiment, the serum albumin binder is AlbX00001 (SEQ ID NO: 334). In one embodiment the serum albumin binder is Alb23002 (SEQ ID NO: 335).

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVDs into a single molecule. The use of linkers to connect two or more (poly) peptides is well known in the art.

In some embodiments, the further exemplary peptidic linkers are shown in Table B. One often used class of peptidic linker are known as the “Gly-Ser” or “GS” linkers. These are linkers that essentially consist of glycine (G) and serine (S) residues, and usually comprise one or more repeats of a peptide motif such as the GGGGS (SEQ ID NO: 337) motif (for example, having the formula (Gly-Gly-Gly-Gly-Ser)n in which n may be 1, 2, 3, 4, 5, 6, 7 or more). Some often-used examples of such GS linkers are 9GS linkers (GGGGSGGGS, SEQ ID NO: 340), 15GS linkers (n=3) and 35GS linkers (n=7). Reference is for example made to Chen et al. 2013 (Adv. Drug Deliv. Rev. 65 (10): 1357-1369) and Klein et al. 2014 (Protein Eng. Des. Sel. 27 (10): 325-330).

TABLE B
Serum albumin binding ISVD sequences, Linker sequences and some proposed ISVD
C-terminal ends (amino acids 109-112 or 109-113, according to Kabat numbering)
(“ID” refers to the SEQ ID NO as used herein)

| Name Seq | |
|---|---|
| ID | Amino acid sequence |
| Alb8 | EVQLVESGGGLVQPGNSLRLSCAASGFTFSSFGMSWVRQAPGKGLEWVSSISGSGSDTLY |
| 320 | ADSVKGRFTISRDNAKTTLYLQMNSLRPEDTAVYYCTIGGSLSRSSQGTLVTVSS |
| Alb23 | EVQLLESGGGLVQPGGSLRLSCAASGFTFRSFGMSWVRQAPGKGPEWVSSISGSGSDTLY |
| 321 | ADSVKGRFTISRDNSKNTLYLQMNSLRPEDTAVYYCTIGGSLSRSSQGTLVTVSS |
| Alb129 | EVQLVESGGGVVQPGNSLRLSCAASGFTFSSFGMSWVRQAPGKGLEWVSSISGSGSDTLY |
| 322 | ADSVKGRFTISRDNAKTTLYLQMNSLRPEDTATYYCTIGGSLSRSSQGTLVTVSSA |
| Alb132 | EVQLVESGGGVVQPGGSLRLSCAASGFTFRSFGMSWVRQAPGKGPEWVSSISGSGSDTL |
| 323 | YADSVKGRFTISRDNSKNTLYLQMNSLRPEDTATYYCTIGGSLSRSSQGTLVTVSSA |
| Alb11 | EVQLVESGGGLVQPGNSLRLSCAASGFTFSSFGMSWVRQAPGKGLEWVSSISGSGSDTLY |
| 324 | ADSVKGRFTISRDNAKTTLYLQMNSLRPEDTAVYYCTIGGSLSRSSQGTLVTVSS |
| Alb11 | EVQLVESGGGLVQPGNSLRLSCAASGFTFSSFGMSWVRQAPGKGLEWVSSISGSGSDTLY |
| (S112K)-A | ADSVKGRFTISRDNAKTTLYLQMNSLRPEDTAVYYCTIGGSLSRSSQGTLVKVSSA |
| 325 | |
| Alb82 | EVQLVESGGGVVQPGNSLRLSCAASGFTFSSFGMSWVRQAPGKGLEWVSSISGSGSDTLY |
| 326 | ADSVKGRFTISRDNAKTTLYLQMNSLRPEDTALYYCTIGGSLSRSSQGTLVTVSS |
| Alb82-A | EVQLVESGGGVVQPGNSLRLSCAASGFTFSSFGMSWVRQAPGKGLEWVSSISGSGSDTLY |
| 327 | ADSVKGRFTISRDNAKTTLYLQMNSLRPEDTALYYCTIGGSLSRSSQGTLVTVSSA |
| Alb82-AA | EVQLVESGGGVVQPGNSLRLSCAASGFTFSSFGMSWVRQAPGKGLEWVSSISGSGSDTLY |
| 328 | ADSVKGRFTISRDNAKTTLYLQMNSLRPEDTALYYCTIGGSLSRSSQGTLVTVSSAA |
| Alb82- | EVQLVESGGGVVQPGNSLRLSCAASGFTFSSFGMSWVRQAPGKGLEWVSSISGSGSDTLY |
| AAA | ADSVKGRFTISRDNAKTTLYLQMNSLRPEDTALYYCTIGGSLSRSSQGTLVTVSSAAA |
| 329 | |
| Alb82-G | EVQLVESGGGVVQPGNSLRLSCAASGFTFSSFGMSWVRQAPGKGLEWVSSISGSGSDTLY |
| 330 | ADSVKGRFTISRDNAKTTLYLQMNSLRPEDTALYYCTIGGSLSRSSQGTLVTVSSG |
| Alb82-GG | EVQLVESGGGVVQPGNSLRLSCAASGFTFSSFGMSWVRQAPGKGLEWVSSISGSGSDTLY |
| 331 | ADSVKGRFTISRDNAKTTLYLQMNSLRPEDTALYYCTIGGSLSRSSQGTLVTVSSGG |
| Alb82- | EVQLVESGGGVVQPGNSLRLSCAASGFTFSSFGMSWVRQAPGKGLEWVSSISGSGSDTLY |
| GGG | ADSVKGRFTISRDNAKTTLYLQMNSLRPEDTALYYCTIGGSLSRSSQGTLVTVSSGGG |
| 332 | |
| Alb223 | EVQLVESGGGVVQPGGSLRLSCAASGFTFRSFGMSWVRQAPGKGPEWVSSISGSGSDTL |
| 333 | YADSVKGRFTISRDNSKNTLYLQMNSLRPEDTALYYCTIGGSLSRSSQGTLVTVSSA |
| AlbX00001 | EVQLVESGGGVVQPGGSLRLSCAASGLTFSSYAMGWFRQAPGKERERVVSISRGGGYTY |
| 334 | YADSVKGRFTISRDNSENTVYLQMNSLRPEDTALYYCAAARYWATGSEYEFDYWGQGT |
| | LVTVSS |
| Alb23002 | EVQLVESGGGVVQPGGSLRLSCAASGFTFRSFGMSWVRQAPGKGPEWVSSISGSGSDTL |
| 335 | YADSVKGRFTISRDNSKNTLYLQMNSLRPEDTALYYCTIGGSLSRSSQGTLVTVSS |
| 336 | AAA |
| 337 | GGGGS |
| 338 | SGGSGGS |
| 339 | GGGGSGGS |
| 340 | GGGGSGGGS |
| 341 | GGGGGGGGS |
| 342 | GGGGSGGGGSGGGGS |
| 343 | GGGGSGGGGSGGGGSGGS |
| 344 | GGGGSGGGGSGGGGSGGGGS |
| 345 | GGGGSGGGGSGGGGSGGGGSGGGGS |
| 346 | GGGGSGGGGSGGGGSGGGGSGGGGSGGGGS |
| 347 | GGGGSGGGGSGGGGSGGGGSGGGGSGGGGSGGGGS |
| 348 | GGGGSGGGGSGGGGSGGGGSGGGGSGGGGSGGGGSGGGGS |
| 349 | EPKSCDKTHTCPPCP |
| 350 | GGGGSGGGSEPKSCDKTHTCPPCP |
| 351 | EPKTPKPQPAAA |
| 352 | ELKTPLGDTTHTCPRCPEPKSCDTPPPCPRCPEPKSCDTPPPCPRCPEPKSCDTPPPCPRCP |
| 353 | VTVSS(X)n |
| 354 | VKVSS |
| 355 | VQVSS |
| 356 | VTVKS |
| 357 | VTVQS |
| 358 | VKVKS |
| 359 | VKVQS |
| 360 | VQVKS |
| 361 | VQVQS |
| 362 | VTVSSA |
| 363 | VKVSSA |
| 364 | VQVSSA |
| 365 | VTVKSA |
| 366 | VTVQSA |
| 367 | VKVKSA |
| 368 | VKVQSA |
| 369 | VQVKSA |
| 370 | VQVQSA |
| 371 | VTVSS |

Specific examples of ISVDs specifically binding to CD8αß are ISVDs that essentially consists of 4 framework regions (FR1 to FR4, respectively) and 3 complementarity determining regions (CDR1 to CDR3, respectively), that interacts with at least one amino acid of the CD8α protein MALPVTALLLPLALLLHAARPSQFRVSPLDRTWNLGETVELKCQVLLSNPTSGCSWLFQ PRGAAASPTFLLYLSQNKPKAAEGLDTQRFSGKRLGDTFVLTLSDFRRENEGYYFCSAL SNSIMYFSHFVPVFLPAKPTTTPAPRPPTPAPTIASQPLSLRPEACRPAAGGAVHTRGLDF ACDIYIWAPLAGTCGVLLLSLVITLYCNHRNRRRVCKCPRPVVKSGDKPSLSARYV (SEQ ID NO: 570) selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570.

In another embodiment, the ISVD interacts with a discontinuous epitope on the CD8α protein.

In another embodiment, the ISVD interacts with at least two amino acids amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least three amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least four amino acids amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least five amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least six amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least seven amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least eight amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least nine amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least ten amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least eleven amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least twelve amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least thirteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least fourteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least fifteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least sixteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least seventeen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least eighteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least nineteen amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570. In another embodiment, the ISVD interacts with at least twenty amino acids of the CD8α protein selected from R²⁵, K42, Q44, V45, L46, L47, S48, N49, P50, T51, S52, G53, C54, L71, Y72, L73, S74, Q75, N76, K77, R⁹³, L94, G95, D96, T97, and D98, when numbered in accordance with SEQ ID NO.: 570.

In another embodiment, the ISVD interacts with an amino acid of the CD8α protein having a buried surface area (BSA) as described herein (e.g. calculated by PDBe-PISA) of more than 10 (R²⁵, K42, Q44, L46, L47, S48, P50, T51, S52, Q75, N76, R⁹³, L94, G95, D96, T97, when numbered in accordance with SEQ ID NO.: 570), more than 25 (R²⁵, Q44, L46, L47, S48, P50, S52, Q75, N76, R⁹³, L94, G95, D96, when numbered in accordance with SEQ ID NO.: 570), preferably more than 50 (R²⁵, L46, P50, Q75, G95, D96, when numbered in accordance with SEQ ID NO.: 570).

In another embodiment, the ISVD interacts with one or more regions of the CD8α protein, when numbered in accordance with SEQ ID NO.: 570, selected from:
region Q44-L46: Q44, V45, and L46;
region L47-C54: L47, S48, N49, P50, T51, S52, G53, and C54;
region L74-K77: S74, Q75, N76, and K77;
region L71-K77: L71, Y72, L73, S74, Q75, N76, and K77; and
region G95-D98: R⁹³, L94, G95, D96, T97, and D98.

In some embodiments, the ISVD interacts with one or more amino acid residues in CD8α, when numbered in accordance with SEQ ID NO.: 570, selected from:
R²⁵, K42, and R⁹³;
R²⁵, V45, L47, S48, Q75, N76, D96, and T97;
R²⁵, K42, L47, S48, Q75, N76, R⁹³, and D96;
R²⁵, K42, V45, L47, S48, Q75, N76, R⁹³, D96, and T97; and
L46 and P50.

In some embodiments, the ISVD interacts with one or more amino acid residues in CD8alpha in region L74-K77 of the CD8α protein, such as N76.

In another embodiment, the ISVD specifically binds to the same amino acid residues and/or the same epitope on the CD8α protein as the ISVD having SEQ ID NO: 169. In this respect, the immunoglobulin single variable domain of the present technology comprises amino acid residues corresponding to the amino acid residues in the ISVD having SEQ ID NO: 169 that interact with the CD8α protein.

Based on the structural data provided herein, the skilled person understands that amino acid residues of an ISVD involved in the interaction with the CD8α protein can be determined and that this interaction site is important for the binding of an ISVD to the epitope on the CD8α protein.

Specifically, the interaction site between an ISVD having SEQ ID NO: 169 and the CD8α protein having SEQ ID NO.: 570 has been determined and provides clear technical guidance for the generation of further ISVDs having the binding specificity of the ISVD having SEQ ID NO: 169, e.g. binding to the same epitope on the CD8α protein.

In this respect, the skilled person understands that an ISVD of the present technology having the binding specificity of the ISVD having SEQ ID NO: 169, e.g. specifically binding to the same epitope on the CD8α protein or blocking the binding to the CD8α protein by an ISVD having SEQ ID NO: 169 is not limited to the specific amino acid sequence of the CDR region and/or the FR region of the ISVD having SEQ ID NO: 169, as long as the ISVD contains at least one or more amino acid residue(s) corresponding to the at least one or more amino acid residue(s) of the ISVD having SEQ ID NO: 169 that interact with the CD8α protein as described herein.

In other words, an ISVD of the present technology having the binding specificity of the ISVD having SEQ ID NO: 169, e.g. specifically binding to the same epitope on the CD8α protein or blocking the binding to the CD8α protein by an ISVD having SEQ ID NO: 169 may contain, as compared to an ISVD having SEQ ID NO: 169, mutations and/or substitutions within the CDR region and/or within the FR region (such as conservative amino acid substitutions) whilst maintaining the amino acids of the ISVD that form the interaction site (i.e., the paratope on the ISVD) with the CD8α protein.

In some embodiments, the amino acids in the ISVD that form part of the interaction site (i.e., the paratope on the ISVD) are selected from F27, T28, F29, E30, D31, Y32 and A33 (Kabat numbering) in CDR1. In some embodiments, the amino acids in the ISVD that form part of the interaction site (i.e., the paratope on the ISVD) are selected from I51, R⁵², T52a, Y53, D54, E55, Q56, T57 and Y58 (Kabat numbering) in the CDR2. In some embodiments, the amino acids in the ISVD that form part of the interaction site (i.e., the paratope on the ISVD) are selected from G95, S96, Y97, Y98, A99, C100, A100a, Y100b, E100j, G100k, V100m, D100n, L100o, and D101 (Kabat numbering) in the CDR3.

In some embodiments, the amino acids in the ISVD that form part of the interaction site (i.e., the paratope on the ISVD) are selected from F27, T28, F29, E30, D31, and Y32 (Kabat numbering) in CDR1. In some embodiments, the amino acids in the ISVD that form part of the interaction site (i.e., the paratope on the ISVD) are selected from I51, R⁵², T52a, Y53, D54, E55, Q56, and T57 (Kabat numbering) in the CDR2. In some embodiments, the amino acids in the ISVD that form part of the interaction site (i.e., the paratope on the ISVD) are selected from G95, S96, Y97, Y98, A99, C100, A100a, (Kabat numbering) in the CDR3.

The interacting amino acids preferably have a distance of less than 4 Å (<4 Å), wherein the distance between the amino acids is measured e.g., in Cryogenic-electron microscopy (cryo-EM).

In some embodiments, the amino acids in the ISVD (i.e., the paratope on the ISVD) that form part of the interaction site of <4 Å are selected from E30, D31, Y32 and A33 (Kabat numbering) in CDR1. In some embodiments, the amino acids in the ISVD (i.e., the paratope on the ISVD) that form part of the interaction site of <4 Å are selected from R⁵², Y53, D54, Q56, and Y58 (Kabat numbering) in the CDR2. In some embodiments, the amino acids in the ISVD (i.e., the paratope on the ISVD) that form part of the interaction site of <4 Å are selected from G95, S96, Y97, Y98, A99, C100, A100a, Y100b, E100j, G100k, V100m, D100n, L100o, and D101 (Kabat numbering) in the CDR3.

In some embodiments the interactions site is a salt bridge that is formed between the amino acids in the ISVD (i.e., the paratope on the ISVD) and the amino acids of the CD8α protein. In some embodiments, the amino acids in the ISVD (i.e., the paratope on the ISVD) that form a salt bridge with the amino acids of the CD8α protein are selected from E30 and D101 (Kabat numbering). In some embodiments, the amino acids in the CD8α protein that form a salt bridge with the amino acids of the ISVD (i.e., the paratope on the ISVD) are selected from R²⁵, K42, R⁹³. In some embodiments, following salt bridges are formed:

| ISVD A044300805_v8 | | Length | Amino acid in hCD8-alpha |
|---|---|---|---|
| Aa residue | Kabat numbering | (Å) | (SEQ ID NO: 570) |
| D31[OD1] | D31[OD1] | 2.9 Å | R25[NH2] |
| D31[OD1] | D31[OD1] | 3.9 Å | K42[NZ] |
| D31[OD2] | D31[OD2] | 3.4 Å | K42[NZ] |
| D120[OD1] | D101[OD1] | 3.4 Å | R93[NH2] |
| D120[OD2] | D101[OD2] | 3.6 Å | R93[NH2] |

[OD1]: one of the two carboxylate oxygen atoms in the acidic side chain of aspartate (D); [OD2]: the second carboxylate oxygen atom in the side chain of aspartate (D); [NH2]: the other terminal nitrogen in the guanidinium group of arginine (R); [NZ]: terminal amino group nitrogen in the basic side chain of lysine (K).

In some embodiments, the immunoglobulin single variable domain (ISVD) specifically binding human CD8alpha comprises D31 and D101, wherein upon binding CD8alpha, following interaction sites of <4 Å are formed:
D31 (Kabat numbering) interacts with R²⁵ of CD8alpha;
D31 (Kabat numbering) interacts with K42 of CD8alpha;
D101 (Kabat numbering) interacts with R⁹³ of CD8alpha.

In some embodiments the interactions site is a hydrogen bond that is formed between the amino acids in the ISVD (i.e., the paratope on the ISVD) and the amino acids of the CD8α protein. In some embodiments, the amino acids in the ISVD (i.e., the paratope on the ISVD) that form a hydrogen bond with the amino acids of the CD8α protein are selected from E30 and D31 (Kabat numbering) in CDR1. In some embodiments, the amino acids in the ISVD (i.e., the paratope on the ISVD) that form a hydrogen bond with the amino acids of the CD8α protein are selected from R⁵² and Q56 (Kabat numbering) in the CDR2. In some embodiments, the amino acids in the ISVD (i.e., the paratope on the ISVD) that form a hydrogen bond with the amino acids of the CD8α protein are selected from S96, Y97, E100j and D100n (Kabat numbering) in the CDR3. In some embodiments, the amino acids in the CD8α protein that form a hydrogen bond with the amino acids of the ISVD (i.e., the paratope on the ISVD) are selected from R²⁵, V45, L47, S48, Q75, N76, D96 and T97. In some embodiments, following hydrogen bonds are formed:

| ISVD A044300805_v8 | | Length | Amino acid in hCD8 |
|---|---|---|---|
| Aa residue | Kabat numbering | (Å) | (SEQ ID NO: 570) |
| R52[NH1] | R52[NH1] | 3.8 Å | V45[O] |
| R52[NH1] | R52[NH1] | 3.6 Å | L47[O] |
| R52[NH2] | R52[NH2] | 3.7 Å | L47[O] |
| Q57[NE2] | Q56[NE2] | 2.9 Å | S48[O] |
| S100[OG] | S96[OG] | 3.2 Å | Q75[OE1] |
| Y101[N] | Y97[N] | 3.5 Å | D96[OD2] |
| E30[O] | E30[O] | 3.3 Å | R25[NH2] |
| D118[O] | D100n[O] | 3.2 Å | Q75[NE2] |
| E114[OE2] | E100j[OE2] | 2.9 Å | N76[ND2] |
| D31[O] | D31[O] | 3.1 Å | D96[N] |
| D31[O] | D31[O] | 3.7 Å | T97[OG1] |

[OD2]: the second carboxylate oxygen atom in the side chain of aspartate (D); [OE1]: one of the two carboxylate oxygen atoms in the side chain of glutamate (E); [OE2]: the second carboxylate oxygen atom in the side chain of glutamate (E); [NH1]: one of the terminal nitrogen atoms in the guanidinium group of arginine (R); [NH2]: the other terminal nitrogen in the guanidinium group of arginine (R); [NE2]: terminal nitrogen in amide of glutamine (Q) or imidazole ring of histidine (H); [ND2]: terminal nitrogen in the side-chain amide group of asparagine (N); [OG]: hydroxyl oxygen atom in the polar side chain of serine (S); [OG1]: hydroxyl oxygen atom in the threonine side chain of threonine (T); [O]: backbone carbonyl oxygen (C═O); [N]: backbone amide nitrogen (—NH).

In some embodiments, the immunoglobulin single variable domain (ISVD) specifically binding human CD8alpha comprises E30, D31, R⁵², Q56, S96, Y97, E100j, and D100n, wherein upon binding CD8alpha, following interaction sites of <4 Å are formed:
R⁵² (Kabat numbering) interacts with V45 of CD8alpha;
R⁵² (Kabat numbering) interacts with L47 of CD8alpha;
Q56 (Kabat numbering) interacts with S48 of CD8alpha;
S96 (Kabat numbering) interacts with Q75 of CD8alpha;
Y97 (Kabat numbering) interacts with D96 of CD8alpha;
E30 (Kabat numbering) interacts with R²⁵ of CD8alpha;
D100n (Kabat numbering) interacts with Q75 of CD8alpha;
E100j (Kabat numbering) interacts with N76 of CD8alpha.
D31 (Kabat numbering) interacts with R⁹⁶ of CD8alpha; and
D31 (Kabat numbering) interacts with T97 of CD8alpha.

In some embodiments the interactions site is a hydrophobic interaction between the amino acids in the ISVD (i.e., the paratope on the ISVD) and the amino acids of the CD8α protein. In some embodiments, the amino acid in the ISVD (i.e., the paratope on the ISVD) that forms a hydrophobic interaction with the amino acids of the CD8α protein is Y98 (Kabat numbering). In some embodiments, the amino acids in the CD8α protein that form a hydrophobic interaction with the amino acids of the ISVD (i.e., the paratope on the ISVD) are selected from L46 and P50.

In some embodiments, the ISVD comprises D31, R⁵², Q56, S96, Y97, E100j, and D101 (Kabat numbering), wherein D31, R⁵², Q56, S96, Y97, E100j, and D101 form an interaction site of <4 Å with at least one amino acid selected from the group consisting of R²⁵, K42, L47, S48, Q75, N76, R⁹³ and D96 of the CD8α protein. In some embodiments, the interaction site is formed with at least two amino acids selected from the group consisting of R²⁵, K42, L47, S48, Q75, N76, R⁹³ and D96 of the CD8α protein. In some embodiments, the interaction site is formed with at least three amino acids selected from the group consisting of R²⁵, K42, L47, S48, Q75, N76, R⁹³ and D96 of the CD8α protein. In some embodiments, the interaction site is formed with at least four amino acids selected from the group consisting of R²⁵, K42, L47, S48, Q75, N76, R⁹³ and D96 of the CD8α protein. In some embodiments, the interaction site is formed with at least five amino acids selected from the group consisting of R²⁵, K42, L47, S48, Q75, N76, R⁹³ and D96 of the CD8α protein. In some embodiments, the interaction site is formed with at least six amino acids selected from the group consisting of R²⁵, K42, L47, S48, Q75, N76, R⁹³ and D96 of the CD8α protein. In some embodiments, the interaction site is formed with at least seven amino acids selected from the group consisting of R²⁵, K42, L47, S48, Q75, N76, R⁹³ and D96 of the CD8α protein. In some embodiments, the interaction site is formed with the amino acids selected from the group consisting of R²⁵, K42, L47, S48, Q75, N76, R⁹³ and D96 of the CD8α protein. In some embodiments, the ISVD comprises E100j (Kabat numbering) and the interaction site is formed with N76 of the CD8α protein.

In some embodiments, the CDR3 is stabilized by a non-canonical disulfide bond between C50 (Kabat numbering) in CDR2 and C100 (Kabat numbering) in CDR3 stabilizing the paratope conformation and creating a rigid binding scaffold that enhances specificity. This covalent linkage constrains CDR3 in a conformation optimal for CD8 recognition. In some embodiments, the ISVD comprises cysteine at position 50 (C50) and cysteine at position 100 (C100) and amino acid residues C50 in CDR2 and C100 in CDR3 are covalently linked via a disulfide bond. The extended CDR3 amino acids 100a-101 (Kabat numbering) are flexible. In some embodiments, the CDR3 (according to Abm definition) has a length of 20 or more, of 21 or more, of 22 or more, of 23 or more, such as of 23 amino acids and comprises E100j (Kabat numbering).

Energetically, P50 and L46 from the CD8α protein contribute most favorably to binding (1.34 and 1.05 kcal/mol), while D31 from ISVD A044300805_v8 provides significant stabilization (1.69 kcal/mol total). This defined epitope-paratope interface underlies ISVD A044300805_v8's selective recognition of CD8 alpha. In some embodiments the ISVD comprises D31. In some embodiment, the ISVD interacts with L46 and P50 on the CD8α protein.

Specific examples of immunoglobulin single variable domains (ISVDs) specifically binding human CD8alpha, that essentially consists of 4 framework regions (FR1 to FR4, respectively) and 3 complementarity determining regions (CDR1 to CDR3, respectively), are ISVDs in which:
CDR1 (according to Abm definition) has an amino acid sequence selected from the group consisting of:
   a) the amino acid sequence of SEQ ID NO: 244;
   b) amino acid sequences that have at least 80% amino acid identity with the amino acid sequence of SEQ ID NO: 244; and
   c) amino acid sequences that have 3, 2, or 1 amino acid difference with the amino acid sequences of SEQ ID NO: 244;
and
CDR2 (according to Abm definition) has an amino acid sequence selected from the group consisting of:
   d) the amino acid sequence of SEQ ID NO: 246;
   e) amino acid sequences that have at least 80% amino acid identity with the amino acid sequence of SEQ ID NO: 246; and
   f) amino acid sequences that have 3, 2, or 1 amino acid difference with the amino acid sequence of SEQ ID NO: 246;
and
CDR3 (according to Abm definition) has an amino acid sequence selected from the group consisting of:
   g) the amino acid sequence of SEQ ID NO: 248;
   h) amino acid sequences that have at least 80% amino acid identity with the amino acid sequence of SEQ ID NO: 248; and
   i) amino acid sequences that have 3, 2, or 1 amino acid difference with the amino acid sequence of SEQ ID NO: 248.

In some embodiments, the immunoglobulin single variable domain (ISVD) specifically binding human CD8alpha essentially consists of 4 framework regions (FR1 to FR4, respectively) and 3 complementarity determining regions (CDR1 to CDR3, respectively), in which:
CDR1 (according to Abm definition) has an amino acid sequence selected from the group consisting of:
   a) the amino acid sequence of SEQ ID NO: 244;
   b) amino acid sequences that have at least 80% amino acid identity with the amino acid sequence of SEQ ID NO: 244; and
   c) amino acid sequences that have 3, 2, or 1 amino acid difference with the amino acid sequences of SEQ ID NO: 244;
   wherein CDR1 comprises at least three amino acid residues selected from the group consisting of F27, T28, F29, E30, D31, Y32 and A33 (Kabat numbering),
   wherein CDR1 comprises at least four amino acid residues selected from the group consisting of F27, T28, F29, E30, D31, Y32 and A33 (Kabat numbering);
   wherein CDR1 comprises at least five amino acid residues selected from the group consisting of F27, T28, F29, E30, D31, Y32 and A33 (Kabat numbering);
   wherein CDR1 comprises at least six amino acid residues selected from the group consisting of F27, T28, F29, E30, D31, Y32 and A33 (Kabat numbering);
   wherein CDR1 comprises F27, T28, F29, E30, D31, Y32 and A33 (Kabat numbering);
   wherein CDR1 comprises at least D31 (Kabat numbering);
   wherein CDR1 comprises at least E30 and D31 (Kabat numbering);
   wherein CDR1 comprises at least E30, D31 and Y32 (Kabat numbering); and/or
   wherein CDR1 comprises at least E30, D31, Y32 and A33 (Kabat numbering);
and
CDR2 (according to Abm definition) has an amino acid sequence selected from the group consisting of:
   d) the amino acid sequence of SEQ ID NO: 246;
   e) amino acid sequences that have at least 80% amino acid identity with the amino acid sequence of SEQ ID NO: 246; and
   f) amino acid sequences that have 3, 2, or 1 amino acid difference with the amino acid sequence of SEQ ID NO: 246;
   wherein CDR2 comprises at least five amino acid residues selected from the group consisting of I51, R⁵², T52a, Y53, D54, E55, Q56, T57 and Y58 (Kabat numbering);
   wherein CDR2 comprises at least six amino acid residues selected from the group consisting of I51, R⁵², T52a, Y53, D54, E55, Q56, T57 and Y58 (Kabat numbering);
   wherein CDR2 comprises at least seven amino acid residues selected from the group consisting of I51, R⁵², T52a, Y53, D54, E55, Q56, T57 and Y58 (Kabat numbering);
   wherein CDR2 comprises at least eight amino acid residues selected from the group consisting of I51, R⁵², T52a, Y53, D54, E55, Q56, T57 and Y58 (Kabat numbering);
   wherein CDR2 comprises I51, R⁵², T52a, Y53, D54, E55, Q56, T57 and Y58 (Kabat numbering);
   wherein CDR2 comprises at least R⁵² and Y53 (Kabat numbering);
   wherein CDR2 comprises at least R⁵² and Q56 (Kabat numbering); and/or
   wherein CDR2 comprises at least R⁵², Y53, D54, Q56 and Y58 (Kabat numbering);
and
CDR3 (according to Abm definition) has an amino acid sequence selected from the group consisting of:
   g) the amino acid sequence of SEQ ID NO: 248;
   h) amino acid sequences that have at least 80% amino acid identity with the amino acid sequence of SEQ ID NO: 248; and
   i) amino acid sequences that have 7, 6, 5, 4, 3, 2, or 1 amino acid difference with the amino acid sequence of SEQ ID NO: 248;
   wherein CDR3 comprises at least 7 amino acid residues selected from the group consisting of G95, S96, Y97, Y98, A99, C100, A100a, Y100b, E100j, G100k, V100m, D100n, L100o, and D101 (Kabat numbering),
   wherein CDR3 comprises at least 8 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 9 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 10 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 11 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 12 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 13 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises G95, S96, Y97, Y98, A99, C100, A100a, Y100b, E100j, G100k, V100m, D100n, L100o, and D101 (Kabat numbering);
   wherein CDR3 comprises at least D101 (Kabat numbering);
   wherein CDR3 comprises at least S96, Y97, D100n, and E100j (Kabat numbering);
   wherein CDR3 comprises at least Y98 (Kabat numbering);
   wherein CDR3 comprises at least S96, Y97, E100j and D101 (Kabat numbering);
   wherein CDR3 comprises at least S96, Y97, D100n, E100j and D101 (Kabat numbering);
   wherein CDR3 comprises at least G95, S96, Y97, Y98, A99, C100, and A100a (Kabat numbering);
   wherein CDR3 comprises at least G95, S96, Y97, Y98, A99, C100, A100a, and Y100b (Kabat numbering); and/or wherein CDR3 comprises at least G95, S96, Y97, Y98, A99, C100, A100a, Y100b, E100j, G100k, V100m, D100n, L100o, and D101 (Kabat numbering).

In some embodiments, the flexible loop of CDR3 100d-100i can be truncated, while E114 is essential to maintain CDR3 conformation. In some embodiments, the immunoglobulin single variable domain (ISVD) comprises a CDR3 (according to Abm definition) comprising at least 20 amino acids, at least 21 amino acids, at least 22 amino acids, at least 23 amino acids, and/or the CDR3 (according to Abm definition) consists of 23 amino acids and position 100j (Kabat numbering) in CDR3 is E.

Examples of ISVDs specifically binding human CD8alpha, that essentially consists of 4 framework regions (FR1 to FR4, respectively) and 3 complementarity determining regions (CDR1 to CDR3, respectively), are ISVDs in which:
CDR1 (according to Abm definition) has an amino acid sequence selected from the group consisting of:
   a) the amino acid sequence of GFTFX₁DYAIG (SEQ ID NO: 571);
   b) amino acid sequences that have at least 80% amino acid identity with the amino acid sequence of GFTFX₁DYAIG (SEQ ID NO: 571); and
   c) amino acid sequences that have 3, 2, or 1 amino acid difference with the amino acid sequences of GFTFX₁DYAIG (SEQ ID NO: 571);
   wherein X₁ is selected from D and E;
and
CDR2 (according to Abm definition) has an amino acid sequence selected from the group consisting of:
   d) the amino acid sequence of CIRTYDX₂X₃TY (SEQ ID NO: 572);
   e) amino acid sequences that have at least 80% amino acid identity with the amino acid sequence of CIRTYDX₂X₃TY (SEQ ID NO: 572); and
   f) amino acid sequences that have 3, 2, or 1 amino acid difference with the amino acid sequence of CIRTYDX₂X₃TY (SEQ ID NO: 572)
   wherein X₂ is selected from G and E;
   wherein X₃ is selected from N and Q;
and
CDR3 (according to Abm definition) has an amino acid sequence selected from the group consisting of:
   g) the amino acid sequence of GSYYACAX₄YSRPDPSEX₅HVDX₆DY (SEQ ID NO: 573);
   h) amino acid sequences that have at least 80% amino acid identity with the amino acid sequence of GSYYACAX₄YSRPDPSEX₅HVDX₆DY (SEQ ID NO: 573); and
   i) amino acid sequences that have 3, 2, or 1 amino acid difference with the amino acid sequence of GSYYACAX₄YSRPDPSEX₅HVDX₆DY (SEQ ID NO: 573);
   wherein X₄ is selected from K, E and Y;
   wherein X₅ is selected from N and G; and/or
   wherein X₆ is selected from M and L.

The SEQ ID NOs for the CDR sequences referred to above are based on the CDR definition according to the AbM definition (see Table C-1). It is noted that the SEQ ID NOs for the CDR sequences defined according to the Kabat definition can likewise be used (see Table C-1). Accordingly, the ISVDs provided by the present technology, specifically binding to CD8alpha as described above by its CDRs using the AbM definition, can be also described by its CDRs using the Kabat definition.

Examples of ISVDs specifically binding human CD8alpha, that essentially consists of 4 framework regions (FR1 to FR4, respectively) and 3 complementarity determining regions (CDR1 to CDR3, respectively), are ISVDs in which:
CDR1 (according to Kabat definition) has an amino acid sequence selected from the group consisting of:
   a) the amino acid sequence of SEQ ID NO: 314;
   b) amino acid sequences that have at least 80% amino acid identity with the amino acid sequence of SEQ ID NO: 314; and
   c) amino acid sequences that have 3, 2, or 1 amino acid difference with the amino acid sequences of SEQ ID NO: 314;
and
CDR2 (according to Kabat definition) has an amino acid sequence selected from the group consisting of:
   d) the amino acid sequence of SEQ ID NO: 316;
   e) amino acid sequences that have at least 80% amino acid identity with the amino acid sequence of SEQ ID NO: 316; and
   f) amino acid sequences that have 3, 2, or 1 amino acid difference with the amino acid sequence of SEQ ID NO: 316;
and
CDR3 (according to Kabat definition) has an amino acid sequence selected from the group consisting of:
   g) the amino acid sequence of SEQ ID NO: 318;
   h) amino acid sequences that have at least 80% amino acid identity with the amino acid sequence of SEQ ID NO: 318;
   i) amino acid sequences that have 3, 2, or 1 amino acid difference with the amino acid sequence of SEQ ID NO: 318.

In some embodiments, the immunoglobulin single variable domain (ISVD) specifically binding human CD8alpha essentially consists of 4 framework regions (FR1 to FR4, respectively) and 3 complementarity determining regions (CDR1 to CDR3, respectively), in which:
CDR1 (according to Kabat definition) has an amino acid sequence selected from the group consisting of:
   a) the amino acid sequence of SEQ ID NO: 314;
   b) amino acid sequences that have at least 80% amino acid identity with the amino acid sequence of SEQ ID NO: 314; and
   c) amino acid sequences that have 3, 2, or 1 amino acid difference with the amino acid sequences of SEQ ID NO: 314;
   wherein CDR1 comprises at least one amino acid residues selected from the group consisting of D31, Y32 and A33 (Kabat numbering);
   wherein CDR1 comprises at least two amino acid residues selected from the group consisting of D31, Y32 and A33 (Kabat numbering);
   wherein CDR1 comprises D31, Y32 and A33 (Kabat numbering);
   wherein CDR1 comprises at least D31 (Kabat numbering);
   wherein CDR1 comprises at least D31 and Y32 (Kabat numbering); and/or wherein CDR1 comprises at least D31, Y32 and A33 (Kabat numbering);
and
CDR2 (according to Kabat definition) has an amino acid sequence selected from the group consisting of:
   d) the amino acid sequence of SEQ ID NO: 316;
   e) amino acid sequences that have at least 80% amino acid identity with the amino acid sequence of SEQ ID NO: 316; and
   f) amino acid sequences that have 5, 4, 3, 2, or 1 amino acid difference with the amino acid sequence of SEQ ID NO: 316;
   wherein CDR2 comprises at least three amino acid residues selected from the group consisting of I51, R⁵², T52a, Y53, D54, E55, Q56, T57 and Y58 (Kabat numbering);
   wherein CDR2 comprises at least four amino acid residues selected from the group consisting of I51, R⁵², T52a, Y53, D54, E55, Q56, T57 and Y58 (Kabat numbering);
   wherein CDR2 comprises at least five amino acid residues selected from the group consisting of I51, R⁵², T52a, Y53, D54, E55, Q56, T57 and Y58 (Kabat numbering);
   wherein CDR2 comprises at least six amino acid residues selected from the group consisting of I51, R⁵², T52a, Y53, D54, E55, Q56, T57 and Y58 (Kabat numbering);
   wherein CDR2 comprises at least seven amino acid residues selected from the group consisting of I51, R⁵², T52a, Y53, D54, E55, Q56, T57 and Y58 (Kabat numbering);
   wherein CDR2 comprises I51, R⁵², T52a, Y53, D54, E55, Q56, T57 and Y58 (Kabat numbering);
   wherein CDR2 comprises at least R⁵² and Y53 (Kabat numbering);
   wherein CDR2 comprises at least R⁵² and Q56 (Kabat numbering);
   wherein CDR2 comprises at least R⁵², Y53, D54, Q56 and Y58 (Kabat numbering);
and
CDR3 (according to Kabat definition) has an amino acid sequence selected from the group consisting of:
   g) the amino acid sequence of SEQ ID NO: 318;
   h) amino acid sequences that have at least 80% amino acid identity with the amino acid sequence of SEQ ID NO: 318;
   i) amino acid sequences that have 3, 2, or 1 amino acid difference with the amino acid sequence of SEQ ID NO: 318;
   wherein CDR3 comprises at least 7 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 8 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 9 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 10 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 11 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 12 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises at least 13 amino acid residues selected from the group consisting of G95, S96, Y97, Y98, A99, C100, A100a, Y100b, E100j, G100k, V100m, D100n, L100o, and D101 (Kabat numbering);
   wherein CDR3 comprises G95, S96, Y97, Y98, A99, C100, A100a, Y100b, E100j, G100k, V100m, D100n, L100o, and D101 (Kabat numbering);
   wherein CDR3 comprises at least D101 (Kabat numbering);
   wherein CDR3 comprises at least S96, Y97, D100n, and E100j (Kabat numbering);
   wherein CDR3 comprises at least Y98 (Kabat numbering);
   wherein CDR3 comprises at least S96, Y97, E100j and D101 (Kabat numbering);
   wherein CDR3 comprises at least S96, Y97, D100n, E100j and D101 (Kabat numbering);
   wherein CDR3 comprises at least G95, S96, Y97, Y98, A99, C100, and A100a (Kabat numbering);
   wherein CDR3 comprises at least G95, S96, Y97, Y98, A99, C100, A100a, and Y100b (Kabat numbering); and/or wherein CDR3 comprises at least G95, S96, Y97, Y98, A99, C100, A100a, Y100b, E100j, G100k, V100m, D100n, L100o, and D101 (Kabat numbering).

Examples of ISVDs specifically binding human CD8alpha that essentially consists of 4 framework regions (FR1 to FR4, respectively) and 3 complementarity determining regions (CDR1 to CDR3, respectively), are ISVDs in which:
CDR1 (according to Kabat definition) has an amino acid sequence selected from the group consisting of:
   a) the amino acid sequence of SEQ ID NO: 314;
   b) amino acid sequences that have at least 80% amino acid identity with the amino acid sequence of SEQ ID NO: 314; and
   c) amino acid sequences that have 3, 2, or 1 amino acid difference with the amino acid sequences of SEQ ID NO: 314;
and
CDR2 (according to Kabat definition) has an amino acid sequence selected from the group consisting of:
   d) the amino acid sequence of CIRTYDX₁X₂TYYX₃DSVKG (SEQ ID NO: 574);
   e) amino acid sequences that have at least 80% amino acid identity with the amino acid sequence of CIRTYDX₁X₂TYYX₃DSVKG (SEQ ID NO: 574); and
   f) amino acid sequences that have 3, 2, or 1 amino acid difference with the amino acid sequence of CIRTYDX₁X₂TYYX₃DSVKG (SEQ ID NO: 574);
   wherein X₁ is selected from G and E;
   wherein X₂ is selected from N and Q;
   wherein X₃ is selected from I and A;
and
CDR3 (according to Kabat definition) has an amino acid sequence selected from the group consisting of:
   g) the amino acid sequence of GSYYACAX₄YSRPDPSEX₅HVDX₆DY (SEQ ID NO: 573);
   h) amino acid sequences that have at least 80% amino acid identity with the amino acid sequence of GSYYACAX₄YSRPDPSEX₅HVDX₆DY (SEQ ID NO: 573); and
   i) amino acid sequences that have 3, 2, or 1 amino acid difference with the amino acid sequence of GSYYACAX₄YSRPDPSEX₅HVDX₆DY (SEQ ID NO: 573);
   wherein X₄ is selected from K, E and Y;
   wherein X₅ is selected from N and G; and/or
   wherein X₆ is selected from M and L.

In one aspect, the disclosure also relates to such ISVDs that can bind to and/or are directed against CD8α (CD8alpha) and that comprise CDR sequences that are generally as further defined herein, to suitable fragments thereof, as well as to polypeptides that comprise or essentially consist of one or more of such ISVDs and/or suitable fragments. In some aspect, the disclosure relates to an ISVDs comprising a sequence selected from the group consisting of SEQ ID NOs: 160 to 179. In particular, the disclosure in some specific aspects provides:
I) ISVDs that are directed against CD8α and that have at least 80%, preferably at least 85%, such as 90% or 95% or more sequence identity with an ISVD comprising a sequence selected from the group consisting of SEQ ID NOs: 160 to 179;
II) ISVDs that cross-block the binding of the amino acid sequence selected from the group consisting of SEQ ID NOs: 160 to 179 to CD8α and/or that compete with at least the ISVD selected from the group consisting of SEQ ID NOs: 160 to 179 for binding to CD8α;
Such ISVDs may be as further described herein (and may for example be VHHs, including humanized VHHs, VHs, including human VHs, camelized VHs and camelized human VHs); as well as polypeptides of the disclosure that comprise one or more of such amino acid sequences (which may be as further described herein), and particularly bispecific (or multispecific) polypeptides as described herein, and nucleic acid sequences that encode ISVDs and polypeptides. Such ISVDs and polypeptides do not include any naturally occurring ligands.

In some embodiments, the CD8α is derived from a mammalian animal, such as a human being. In one specific, but non-limiting aspect, the disclosure relates to an ISVD directed against CD8α, that comprises:
a) the amino acid sequence selected from the group consisting of SEQ ID NOs: 160 to 179;
b) amino acid sequences that have at least 80% amino acid identity with a sequence selected from the group consisting of SEQ ID NOs: 160 to 179, or
c) amino acid sequences that have 3, 2, or 1 amino acid difference with a sequence selected from the group consisting of SEQ ID NOs: 160 to 179;
or any suitable combination thereof.

In some embodiments, disclosed is an ISVD against CD8α, which consist of 4 framework regions (FR1 to FR4 respectively) and 3 complementarity determining regions (CDR1 to CDR3 respectively). In some embodiments, in such an ISVD:
(I) CDR1 comprises or essentially consists of an amino acid sequence selected from the group consisting of SEQ ID NOs: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition;
or amino acid sequences that have at least 80%, at least 90%, at least 95%, at least 99% or more sequence identity with a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition;
and/or amino acids sequences that have 2 or only 1 amino acid difference(s) with a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition, in which
any amino acid substitution is a conservative amino acid substitution; and/or
said amino acid sequence only contains amino acid substitutions, and no amino acid deletions or insertions, compared to a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition.
(II) CDR2 comprises or essentially consists of an amino acid sequence comprising a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition,
or amino acid sequences that have at least 80%, at least 90%, at least 95%, at least 99% or more sequence identity with a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition;
and/or amino acids sequences that have 2 or only 1 amino acid difference(s) with a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition in which
any amino acid substitution is a conservative amino acid substitution; and/or
said amino acid sequence only contains amino acid substitutions, and no amino acid deletions or insertions, compared to a sequence selected from the group consisting of SEQ ID NO: 183, 190, 197, 204, 211, 218, 225, 232, 239 and 246 according to Abm definition and SEQ ID NOs: 253, 260, 267, 274, 281, 288, 295, 302, 309 and 316 according to Kabat definition.
(III) CDR3 comprises or essentially consists of an amino acid sequence comprising a sequence selected from the group consisting of SEQ ID NO: 185, 192, 199, 206, 213, 220, 227, 234, 241 and 248 according to Abm definition, and SEQ ID NOs: 255, 262, 269, 276, 283, 290, 297, 304, 311 and 318 according to Kabat definition,
or amino acid sequences that have at least 80%, at least 90%, at least 95%, at least 99% or more sequence identity with a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition;
and/or from the group consisting of amino acids sequences that have 2 or only 1 amino acid difference(s) with a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition, in which any amino acid substitution is a conservative amino acid substitution; and/or said amino acid sequence only contains amino acid substitutions, and no amino acid deletions or insertions, compared to a sequence selected from the group consisting of SEQ ID NO: 185, 192, 199, 206, 213, 220, 227, 234, 241 and 248 according to Abm definition and SEQ ID NOs: 4 255, 262, 269, 276, 283, 290, 297, 304, 311 and 318 according to Kabat definition.

In one embodiment, the ISVD comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (according to Abm definition) or that comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (according to Kabat definition).

CD8 binding ISVDs as disclosed herein may comprise one, two or all three of the CDRs explicitly listed above. In some embodiments, the anti-CD8α ISVD is selected from the ISVDs described in the tables below. In some embodiments, the anti-CD8αISVD is selected from the ISVDs that have at least 80%, at least 85%, at least 90%, at least 95%, at least 96%, at least 97%, at least 98%, or at least 99% sequence identity with the ISVDs described Table C-1, Table C-2, and Table C-3. In some embodiments, the anti-CD8 ISVD is selected from the ISVDs that have at least 80%, at least 85%, at least 90%, at least 95%, at least 96%, at least 97%, at least 98%, or at least 99% sequence identity with the ISVDs described in Table C-4.

TABLE C-1
Anti-CD8α ISVDs

| | Full | fw 1 | cdr1 | fw 2 | cdr 2 | fw3 | cdr3 | fw4 |
|---|---|---|---|---|---|---|---|---|
| name | SEQ ID | SEQ ID | SEQ ID | SEQ ID | SEQ ID | SEQ ID | SEQ ID | SEQ ID |
| Abm CDR definition | | | | | | | | |
| T0347015C01 (15C01) | 160 | 180 | 181 | 182 | 183 | 184 | 185 | 186 |
| A044300805 | 161 | 187 | 188 | 189 | 190 | 191 | 192 | 193 |
| A044300805_v1 (15C01v1) | 162 | 194 | 195 | 196 | 197 | 198 | 199 | 200 |
| A044300805_v2 (15C01v2) | 163 | 201 | 202 | 203 | 204 | 205 | 206 | 207 |
| A044300805_v3 (15C01v3) | 164 | 208 | 209 | 210 | 211 | 212 | 213 | 214 |
| A044300805_v4 (15C01v4) | 165 | 215 | 216 | 217 | 218 | 219 | 220 | 221 |
| A044300805_v5 (15C01v5) | 166 | 222 | 223 | 224 | 225 | 226 | 227 | 228 |
| A044300805_v6 (15C01v6) | 167 | 229 | 230 | 231 | 232 | 233 | 234 | 235 |
| A044300805_v7 (15C01v7) | 168 | 236 | 237 | 238 | 239 | 240 | 241 | 242 |
| A044300805_v8 (15C01v8) | 169 | 243 | 244 | 245 | 246 | 247 | 248 | 249 |
| Kabat CDR definition | | | | | | | | |
| T0347015C01 (15C01) | 170 | 250 | 251 | 252 | 253 | 254 | 255 | 256 |
| A044300805 | 171 | 257 | 258 | 259 | 260 | 261 | 262 | 263 |
| A044300805_v1 (15C01v1) | 172 | 264 | 265 | 266 | 267 | 268 | 269 | 270 |
| A044300805_v2 (15C01v2) | 173 | 271 | 272 | 273 | 274 | 275 | 276 | 277 |
| A044300805_v3 (15C01v3) | 174 | 278 | 279 | 280 | 281 | 282 | 283 | 284 |
| A044300805_v4 (15C01v4) | 175 | 285 | 286 | 287 | 288 | 289 | 290 | 291 |
| A044300805_v5 (15C01v5) | 176 | 292 | 293 | 294 | 295 | 296 | 297 | 298 |
| A044300805_v6 (15C01v6) | 177 | 299 | 300 | 301 | 302 | 303 | 304 | 305 |
| A044300805_v7 (15C01v7) | 178 | 306 | 307 | 308 | 309 | 310 | 311 | 312 |
| A044300805_v8 (15C01v8) | 179 | 313 | 314 | 315 | 316 | 317 | 318 | 319 |
| IMGT CDR definition | | | | | | | | |
| BDSn | 419 | 420 | 421 | 422 | 423 | 424 | 425 | 426 |

TABLE C-2
Anti-CD8a ISVD full sequences

| Seq | |
|---|---|
| ID | Full seq |
| 160 | EVQLVESGGGLVQAGGSLRLSCAASGFTFDDYAIGWFRQAPGKEREEVSCIRTYDGNTYYIDSV |
| | KGRFTISSDNAKNTVYLQMNSLKPEDTAAYYCAAGSYYACAKYSRPDPSENHVDMDYWGKGT |
| | LVTVSS |
| 161 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAKYSRPDPSEGHVDLDYWGQGTL |
| | VTVSS |
| 162 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAEYSRPDPSEGHVDLDYWGQGTL |
| | VTVSS |
| 163 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAYYSRPDPSEGHVDLDYWGQGTL |
| | VTVSS |
| 164 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAKYSRPDPSEGHVDLDYWGQGTL |
| | VTVSS |
| 165 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAEYSRPDPSEGHVDLDYWGQGTL |
| | VTVSS |
| 166 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAYYSRPDPSEGHVDLDYWGQGTL |
| | VTVSS |
| 167 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAKYSRPDPSEGHVDLDYWGQGTL |
| | VTVSS |
| 168 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAEYSRPDPSEGHVDLDYWGQGTL |
| | VTVSS |
| 169 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAYYSRPDPSEGHVDLDYWGQGTL |
| | VTVSS |
| 170 | EVQLVESGGGLVQAGGSLRLSCAASGFTFDDYAIGWFRQAPGKEREEVSCIRTYDGNTYYIDSV |
| | KGRFTISSDNAKNTVYLQMNSLKPEDTAAYYCAAGSYYACAKYSRPDPSENHVDMDYWGKGT |
| | LVTVSS |
| 171 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAKYSRPDPSEGHVDLDYWGQGTL |
| | VTVSS |
| 172 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAEYSRPDPSEGHVDLDYWGQGTL |
| | VTVSS |
| 173 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAYYSRPDPSEGHVDLDYWGQGTL |
| | VTVSS |
| 174 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAKYSRPDPSEGHVDLDYWGQGTL |
| | VTVSS |
| 175 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAEYSRPDPSEGHVDLDYWGQGTL |
| | VTVSS |
| 176 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAYYSRPDPSEGHVDLDYWGQGTL |
| | VTVSS |
| 177 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAKYSRPDPSEGHVDLDYWGQGTL |
| | VTVSS |
| 178 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAEYSRPDPSEGHVDLDYWGQGTL |
| | VTVSS |
| 179 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAYYSRPDPSEGHVDLDYWGQGTL |
| | VTVSS |
| 28 | EVQLVESGGGLVQAGGSLRLSCAASGFTFDDYAIGWFRQAPGKEREEVSCIRTYDGNTYYIDSV |
| | KGRFTISSDNAKNTVYLQMNSLKPEDTAAYYCAAGSYYACAKYSRPDPSENHVDMDYWGKGT |
| | LVTVSSGGC |
| 29 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAKYSRPDPSEGHVDLDYWGQGTL |
| | VTVSSGGC |
| 30 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAEYSRPDPSEGHVDLDYWGQGTL |
| | VTVSSGGC |
| 31 | EVQLVESGGGVVQPGGSLRLSCAASGFTFEDYAIGWFRQAPGKEREEVSCIRTYDEQTYYADSV |
| | KGRFTISRDNAKNTVYLQMNSLRPEDTALYYCAAGSYYACAYYSRPDPSEGHVDLDYWGQGTL |
| | VTVSSGGC |
| 32 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAKYSRPDPSEGHVDLDYWGQGTL |
| | VTVSSGGC |
| 33 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAEYSRPDPSEGHVDLDYWGQGTL |
| | VTVSSGGC |
| 34 | EVQLVESGGGVVQPGGSLRLSCAASGFTFEDYAIGWFRQAPGKEREEVSCIRTYDEQTYYADSV |
| | KGRFTISRDNAKNTVDLQMNSLRPEDTALYYCAAGSYYACAYYSRPDPSEGHVDLDYWGQGTL |
| | VTVSSGGC |
| 35 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAKYSRPDPSEGHVDLDYWGQGTL |
| | VTVSSGGC |
| 36 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAEYSRPDPSEGHVDLDYWGQGTL |
| | VTVSSGGC |
| 44 | EVQLVESGGGVVQPGGSLRLSCAASGFTFEDYAIGWFRQAPGKEREEVSCIRTYDEQTYYADSV |
| | KGRFTISRDNAKNTVSLQMNSLRPEDTALYYCAAGSYYACAYYSRPDPSEGHVDLDYWGQGTL |
| | VTVSSGGC |

TABLE C-3
anti-CD8α ISVD CDRs

| SEQ | | Seq | | Seq | | Seq | | Seq | | Seq | | Seq | |
|---|---|---|---|---|---|---|---|---|---|---|---|---|---|
| ID | Fw1 | ID | cdr1 | ID | fw2 | ID | cdr2 | ID | fw3 | ID | cdr3 | ID | fw4 |
| 180 | EVQLVESGGGL | 181 | GFTF | 182 | WFRQ | 183 | CIRTYD | 184 | YIDSVKGRFTISSDNA | 185 | GSYYACAK | 186 | WGK |
| | VQAGGSLRLSC | | DDY | | APGKE | | GNTY | | KNTVYLQMNSLKPE | | YSRPDPSEN | | GTLV |
| | AAS | | AIG | | REEVS | | | | DTAAYYCAA | | HVDMDY | | TVSS |
| 187 | EVQLVESGGGV | 188 | GFTF | 189 | WFRQ | 190 | CIRTYDE | 191 | YADSVKGRFTISRDN | 192 | GSYYACAK | 193 | WGQ |
| | VQPGGSLRLSC | | EDYA | | APGKE | | QTY | | AKNTVYLQMNSLRP | | YSRPDPSEG | | GTLV |
| | AAS | | IG | | REEVS | | | | EDTALYYCAA | | HVDLDY | | TVSS |
| 194 | EVQLVESGGGV | 195 | GFTF | 196 | WFRQ | 197 | CIRTYDE | 198 | YADSVKGRFTISRDN | 199 | GSYYACAE | 200 | WGQ |
| | VQPGGSLRLSC | | EDYA | | APGKE | | QTY | | AKNTVYLQMNSLRP | | YSRPDPSEG | | GTLV |
| | AAS | | IG | | REEVS | | | | EDTALYYCAA | | HVDLDY | | TVSS |
| 201 | EVQLVESGGGV | 202 | GFTF | 203 | WFRQ | 204 | CIRTYDE | 205 | YADSVKGRFTISRDN | 206 | GSYYACAY | 207 | WGQ |
| | VQPGGSLRLSC | | EDYA | | APGKE | | QTY | | AKNTVYLQMNSLRP | | YSRPDPSEG | | GTLV |
| | AAS | | IG | | REEVS | | | | EDTALYYCAA | | HVDLDY | | TVSS |
| 208 | EVQLVESGGGV | 209 | GFTF | 210 | WFRQ | 211 | CIRTYDE | 212 | YADSVKGRFTISRDN | 213 | GSYYACAK | 214 | WGQ |
| | VQPGGSLRLSC | | EDYA | | APGKE | | QTY | | AKNTVDLQMNSLRP | | YSRPDPSEG | | GTLV |
| | AAS | | IG | | REEVS | | | | EDTALYYCAA | | HVDLDY | | TVSS |
| 215 | EVQLVESGGGV | 216 | GFTF | 217 | WFRQ | 218 | CIRTYDE | 219 | YADSVKGRFTISRDN | 220 | GSYYACAE | 221 | WGQ |
| | VQPGGSLRLSC | | EDYA | | APGKE | | QTY | | AKNTVDLQMNSLRP | | YSRPDPSEG | | GTLV |
| | AAS | | IG | | REEVS | | | | EDTALYYCAA | | HVDLDY | | TVSS |
| 222 | EVQLVESGGGV | 223 | GFTF | 224 | WFRQ | 225 | CIRTYDE | 226 | YADSVKGRFTISRDN | 227 | GSYYACAY | 228 | WGQ |
| | VQPGGSLRLSC | | EDYA | | APGKE | | QTY | | AKNTVDLQMNSLRP | | YSRPDPSEG | | GTLV |
| | AAS | | IG | | REEVS | | | | EDTALYYCAA | | HVDLDY | | TVSS |
| 229 | EVQLVESGGGV | 230 | GFTF | 231 | WFRQ | 232 | CIRTYDE | 233 | YADSVKGRFTISRDN | 234 | GSYYACAK | 235 | WGQ |
| | VQPGGSLRLSC | | EDYA | | APGKE | | QTY | | AKNTVSLQMNSLRP | | YSRPDPSEG | | GTLV |
| | AAS | | IG | | REEVS | | | | EDTALYYCAA | | HVDLDY | | TVSS |
| 236 | EVQLVESGGGV | 237 | GFTF | 238 | WFRQ | 239 | CIRTYDE | 240 | YADSVKGRFTISRDN | 241 | GSYYACAE | 242 | WGQ |
| | VQPGGSLRLSC | | EDYA | | APGKE | | QTY | | AKNTVSLQMNSLRP | | YSRPDPSEG | | GTLV |
| | AAS | | IG | | REEVS | | | | EDTALYYCAA | | HVDLDY | | TVSS |
| 243 | EVQLVESGGGV | 244 | GFTF | 245 | WFRQ | 246 | CIRTYDE | 247 | YADSVKGRFTISRDN | 248 | GSYYACAY | 249 | WGQ |
| | VQPGGSLRLSC | | EDYA | | APGKE | | QTY | | AKNTVSLQMNSLRP | | YSRPDPSEG | | GTLV |
| | AAS | | IG | | REEVS | | | | EDTAL YYCAA | | HVDLDY | | TVSS |
| 250 | EVQLVESGGGL | 251 | DYAI | 252 | WFRQ | 253 | CIRTYD | 254 | RFTISSDNAKNTVYL | 255 | GSYYACAK | 256 | WGK |
| | VQAGGSLRLSC | | G | | APGKE | | GNTYYI | | QMNSLKPEDTAAYY | | YSRPDPSEN | | GTLV |
| | AASGFTFD | | | | REEVS | | DSVKG | | CAA | | HVDMDY | | TVSS |
| 257 | EVQLVESGGGV | 258 | DYAI | 259 | WFRQ | 260 | CIRTYDE | 261 | RFTISRDNAKNTVYL | 262 | GSYYACAK | 263 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 264 | EVQLVESGGGV | 265 | DYAI | 266 | WFRQ | 267 | CIRTYDE | 268 | RFTISRDNAKNTVYL | 269 | GSYYACAE | 270 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 271 | EVQLVESGGGV | 272 | DYAI | 273 | WFRQ | 274 | CIRTYDE | 275 | RFTISRDNAKNTVYL | 276 | GSYYACAY | 277 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 278 | EVQLVESGGGV | 279 | DYAI | 280 | WFRQ | 281 | CIRTYDE | 282 | RFTISRDNAKNTVDL | 283 | GSYYACAK | 284 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 285 | EVQLVESGGGV | 286 | DYAI | 287 | WFRQ | 288 | CIRTYDE | 289 | RFTISRDNAKNTVDL | 290 | GSYYACAE | 291 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 292 | EVQLVESGGGV | 293 | DYAI | 294 | WERQ | 295 | CIRTYDE | 296 | RFTISRDNAKNTVDL | 297 | GSYYACAY | 298 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 299 | EVQLVESGGGV | 300 | DYAI | 301 | WFRQ | 302 | CIRTYDE | 303 | RFTISRDNAKNTVSL | 304 | GSYYACAK | 305 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 306 | EVQLVESGGGV | 307 | DYAI | 308 | WFRQ | 309 | CIRTYDE | 310 | RFTISRDNAKNTVSL | 311 | GSYYACAE | 312 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |
| 313 | EVQLVESGGGV | 314 | DYAI | 315 | WFRQ | 316 | CIRTYDE | 317 | RFTISRDNAKNTVSL | 318 | GSYYACAY | 319 | WGQ |
| | VQPGGSLRLSC | | G | | APGKE | | QTYYAD | | QMNSLRPEDTALYY | | YSRPDPSEG | | GTLV |
| | AASGFTFE | | | | REEVS | | SVKG | | CAA | | HVDLDY | | TVSS |

In one embodiment, the ISVD comprises a CDR1 (according to AbM definition) that is the amino acid sequence of SEQ ID NO: 181, a CDR2 (according to AbM definition) that is the amino acid sequence of SEQ ID NO: 183, and a CDR3 (according to AbM definition) that is the amino acid sequence of SEQ ID NO: 185.

In one embodiment, the ISVD comprises a CDR1 (according to AbM definition) that is the amino acid sequence of SEQ ID NO: 188, a CDR2 (according to AbM definition) that is the amino acid sequence of SEQ ID NO: 190, and a CDR3 (according to AbM definition) that is the amino acid sequence of SEQ ID NO: 192.

In one embodiment, the ISVD comprises a CDR1 (according to AbM definition) that is the amino acid sequence of SEQ ID NO: 195, a CDR2 (according to AbM definition) that is the amino acid sequence of SEQ ID NO: 197, and a CDR3 (according to AbM definition) that is the amino acid sequence of SEQ ID NO: 199.

In one embodiment, the ISVD comprises a CDR1 (according to AbM definition) that is the amino acid sequence of SEQ ID NO: 202, a CDR2 (according to AbM definition) that is the amino acid sequence of SEQ ID NO: 204, and a CDR3 (according to AbM definition) that is the amino acid sequence of SEQ ID NO: 206.

In one embodiment, the ISVD comprises a CDR1 (according to AbM definition) that is the amino acid sequence of SEQ ID NO: 209, a CDR2 (according to AbM definition) that is the amino acid sequence of SEQ ID NO: 211, and a CDR3 (according to AbM definition) that is the amino acid sequence of SEQ ID NO: 213.

In one embodiment, the ISVD comprises a CDR1 (according to AbM definition) that is the amino acid sequence of SEQ ID NO: 216, a CDR2 (according to AbM definition) that is the amino acid sequence of SEQ ID NO: 218, and a CDR3 (according to AbM definition) that is the amino acid sequence of SEQ ID NO: 220.

In one embodiment, the ISVD comprises a CDR1 (according to AbM definition) that is the amino acid sequence of SEQ ID NO: 223, a CDR2 (according to AbM definition) that is the amino acid sequence of SEQ ID NO: 225, and a CDR3 (according to AbM definition) that is the amino acid sequence of SEQ ID NO: 227.

In one embodiment, the ISVD comprises a CDR1 (according to AbM definition) that is the amino acid sequence of SEQ ID NO: 230, a CDR2 (according to AbM definition) that is the amino acid sequence of SEQ ID NO: 232, and a CDR3 (according to AbM definition) that is the amino acid sequence of SEQ ID NO: 234.

In one embodiment, the ISVD comprises a CDR1 (according to AbM definition) that is the amino acid sequence of SEQ ID NO: 237, a CDR2 (according to AbM definition) that is the amino acid sequence of SEQ ID NO: 239, and a CDR3 (according to AbM definition) that is the amino acid sequence of SEQ ID NO: 241.

In one embodiment, the ISVD comprises a CDR1 (according to AbM definition) that is the amino acid sequence of SEQ ID NO: 244, a CDR2 (according to AbM definition) that is the amino acid sequence of SEQ ID NO: 246, and a CDR3 (according to AbM definition) that is the amino acid sequence of SEQ ID NO: 248.

In one embodiment, the ISVD comprises a CDR1 (according to Kabat definition) that is the amino acid sequence of SEQ ID NO: 251, a CDR2 (according to Kabat definition) that is the amino acid sequence of SEQ ID NO: 253, and a CDR3 (according to Kabat definition) that is the amino acid sequence of SEQ ID NO: 255.

In one embodiment, the ISVD comprises a CDR1 (according to Kabat definition) that is the amino acid sequence of SEQ ID NO: 258, a CDR2 (according to Kabat definition) that is the amino acid sequence of SEQ ID NO: 260, and a CDR3 (according to Kabat definition) that is the amino acid sequence of SEQ ID NO: 262.

In one embodiment, the ISVD comprises a CDR1 (according to Kabat definition) that is the amino acid sequence of SEQ ID NO: 265, a CDR2 (according to Kabat definition) that is the amino acid sequence of SEQ ID NO: 267, and a CDR3 (according to Kabat definition) that is the amino acid sequence of SEQ ID NO: 269.

In one embodiment, the ISVD comprises a CDR1 (according to Kabat definition) that is the amino acid sequence of SEQ ID NO: 272, a CDR2 (according to Kabat definition) that is the amino acid sequence of SEQ ID NO: 274, and a CDR3 (according to Kabat definition) that is the amino acid sequence of SEQ ID NO: 276.

In one embodiment, the ISVD comprises a CDR1 (according to Kabat definition) that is the amino acid sequence of SEQ ID NO: 279, a CDR2 (according to Kabat definition) that is the amino acid sequence of SEQ ID NO: 281, and a CDR3 (according to Kabat definition) that is the amino acid sequence of SEQ ID NO: 283.

In one embodiment, the ISVD comprises a CDR1 (according to Kabat definition) that is the amino acid sequence of SEQ ID NO: 286, a CDR2 (according to Kabat definition) that is the amino acid sequence of SEQ ID NO: 288, and a CDR3 (according to Kabat definition) that is the amino acid sequence of SEQ ID NO: 290.

In one embodiment, the ISVD comprises a CDR1 (according to Kabat definition) that is the amino acid sequence of SEQ ID NO: 293, a CDR2 (according to Kabat definition) that is the amino acid sequence of SEQ ID NO: 295, and a CDR3 (according to Kabat definition) that is the amino acid sequence of SEQ ID NO: 297.

In one embodiment, the ISVD comprises a CDR1 (according to Kabat definition) that is the amino acid sequence of SEQ ID NO: 300, a CDR2 (according to Kabat definition) that is the amino acid sequence of SEQ ID NO: 302, and a CDR3 (according to Kabat definition) that is the amino acid sequence of SEQ ID NO: 304.

In one embodiment, the ISVD comprises a CDR1 (according to Kabat definition) that is the amino acid sequence of SEQ ID NO: 307, a CDR2 (according to Kabat definition) that is the amino acid sequence of SEQ ID NO: 309, and a CDR3 (according to Kabat definition) that is the amino acid sequence of SEQ ID NO: 311.

In one embodiment, the ISVD comprises a CDR1 (according to Kabat definition) that is the amino acid sequence of SEQ ID NO: 314, a CDR2 (according to Kabat definition) that is the amino acid sequence of SEQ ID NO: 316, and a CDR3 (according to Kabat definition) that is the amino acid sequence of SEQ ID NO: 318.

Specific examples of such ISVDs that specifically bind to CD8alpha have one or more, or all, framework regions as indicated for T0347015C₀₁ (SEQ ID NO: 160), and sequence optimized variants thereof in Table C-3 (in addition to the CDRs as defined above). In one embodiment, the ISVD comprises or consists of the full amino acid sequence of T0347015C01 (SEQ ID NO: 160), A044300805 (SEQ ID NO: 161), A044300805_v1 (SEQ ID NO: 162), A044300805_v2 (SEQ ID NO: 163), A044300805_v3 (SEQ ID NO: 164), A044300805_v4 (SEQ ID NO: 165), A044300805_v5 (SEQ ID NO: 166), A044300805_v6 (SEQ ID NO: 167), A044300805_v7 (SEQ ID NO: 168), or A044300805_v8 (SEQ ID NO: 169) (see Table C-1, C-2 and C-3).

In another embodiment, the ISVD specifically binding to human CD8alpha may have a sequence identity of more than 90%, such as more than 95% or even more than 99%, with SEQ ID NO: 160, wherein the CDRs are as defined above. In one embodiment, the ISVD specifically binding to CD8alpha comprises or consists of the amino acid sequence of SEQ ID NO: 160.

In another embodiment, the ISVD specifically binding to human CD8alpha may have a sequence identity of more than 90%, such as more than 95% or even more than 99%, with SEQ ID NO: 161, wherein the CDRs are as defined above. In one embodiment, the ISVD specifically binding to CD8alpha comprises or consists of the amino acid sequence of SEQ ID NO: 161.

In another embodiment, the ISVD specifically binding to human CD8alpha may have a sequence identity of more than 90%, such as more than 95% or even more than 99%, with SEQ ID NO: 162, wherein the CDRs are as defined above. In one embodiment, the ISVD specifically binding to CD8alpha comprises or consists of the amino acid sequence of SEQ ID NO: 162.

In another embodiment, the ISVD specifically binding to human CD8alpha may have a sequence identity of more than 90%, such as more than 95% or even more than 99%, with SEQ ID NO: 163, wherein the CDRs are as defined above. In one embodiment, the ISVD specifically binding to CD8alpha comprises or consists of the amino acid sequence of SEQ ID NO: 163.

In another embodiment, the ISVD specifically binding to human CD8alpha may have a sequence identity of more than 90%, such as more than 95% or even more than 99%, with SEQ ID NO: 164, wherein the CDRs are as defined above. In one embodiment, the ISVD specifically binding to CD8alpha comprises or consists of the amino acid sequence of SEQ ID NO: 164.

In another embodiment, the ISVD specifically binding to human CD8alpha may have a sequence identity of more than 90%, such as more than 95% or even more than 99%, with SEQ ID NO: 165, wherein the CDRs are as defined above. In one embodiment, the ISVD specifically binding to CD8alpha comprises or consists of the amino acid sequence of SEQ ID NO: 165.

In another embodiment, the ISVD specifically binding to human CD8alpha may have a sequence identity of more than 90%, such as more than 95% or even more than 99%, with SEQ ID NO: 166, wherein the CDRs are as defined above. In one embodiment, the ISVD specifically binding to CD8alpha comprises or consists of the amino acid sequence of SEQ ID NO: 166.

In another embodiment, the ISVD specifically binding to human CD8alpha may have a sequence identity of more than 90%, such as more than 95% or even more than 99%, with SEQ ID NO: 167, wherein the CDRs are as defined above. In one embodiment, the ISVD specifically binding to CD8alpha comprises or consists of the amino acid sequence of SEQ ID NO: 167.

In another embodiment, the ISVD specifically binding to human CD8alpha may have a sequence identity of more than 90%, such as more than 95% or even more than 99%, with SEQ ID NO: 168, wherein the CDRs are as defined above. In one embodiment, the ISVD specifically binding to CD8alpha comprises or consists of the amino acid sequence of SEQ ID NO: 168.

In another embodiment, the ISVD specifically binding to human CD8alpha may have a sequence identity of more than 90%, such as more than 95% or even more than 99%, with SEQ ID NO: 169, wherein the CDRs are as defined above. In one embodiment, the ISVD specifically binding to CD8alpha comprises or consists of the amino acid sequence of SEQ ID NO: 169.

In another embodiment, when such an ISVD specifically binding to CD8alpha has 3, 2 or 1 amino acid difference in at least one CDR relative to a corresponding reference CDR sequence (above), the ISVD has at least (essentially) the same binding affinity to human CD8alpha compared to the ISVD with SEQ ID NOs: 169, wherein the binding affinity is measured using the same method, such as e.g. SPR.

In another embodiment, when such an ISVD specifically binding to CD8alpha has 3, 2 or 1 amino acid difference in at least one CDR relative to a corresponding reference CDR sequence (above), the ISVD has at least (essentially) the same binding affinity to cyno CD8alpha compared to ISVD with SEQ ID NOs: 169, wherein the binding affinity is measured using the same method, such as e.g. SPR.

In another embodiment, when such an ISVD specifically binding to CD8alpha has 3, 2 or 1 amino acid difference in at least one CDR relative to a corresponding reference CDR sequence (above), the ISVD still specifically binds to human CD19 and cyno CD19 with less than 10-fold difference in affinity (K_{D}).

In another embodiment, when such an ISVD specifically binding to CD8alpha has 3, 2 or 1 amino acid difference in at least one CDR relative to a corresponding reference CDR sequence (above), the ISVD has at least (essentially) the same potency for binding to CD8+ T cells compared to the ISVD with SEQ ID NOs: 169, wherein the potency is monitored via FACS binding assay e.g., the FACS assay on human T cells (purified from peripheral blood monocytes) as described in the examples.

In another embodiment, when the CDRs of such an ISVD specifically binding to CD8alpha have least 90% amino acid sequence identity, at least 95% amino acid sequence identity, such as 99% amino acid sequence identity or more, with a corresponding reference CDR sequence (above), the ISVD has at least (essentially) the same binding affinity to human CD8alpha compared to the ISVD with SEQ ID NOs: 169, wherein the binding affinity is measured using the same method, such as e.g. SPR.

In another embodiment, when the CDRs of such an ISVD specifically binding to CD8alpha have least 90% amino acid sequence identity, at least 95% amino acid sequence identity, such as 99% amino acid sequence identity or more, with a corresponding reference CDR sequence (above), the ISVD has at least (essentially) the same binding affinity to cyno CD8alpha compared to the ISVD with SEQ ID NOs: 169, wherein the binding affinity is measured using the same method, such as e.g. SPR.

In another embodiment, when the CDRs of such an ISVD specifically binding to CD8alpha have least 90% amino acid sequence identity, at least 95% amino acid sequence identity, such as 99% amino acid sequence identity or more, with a corresponding reference CDR sequence (above), the ISVD still specifically binds to human CD8alpha and cyno CD8alpha with less than 10-fold difference in affinity (K_{D}).

In another embodiment, when the CDRs of such an ISVD specifically binding to CD8alpha have least 90% amino acid sequence identity, at least 95% amino acid sequence identity, such as 99% amino acid sequence identity or more, with a corresponding reference CDR sequence (above), the ISVD has at least (essentially) the same potency for binding to CD8+ T cells compared to the ISVD with SEQ ID NOs: 169, wherein the potency is monitored via FACS binding assay e.g., the FACS assay on human T cells (purified from peripheral blood monocytes) as described in the examples.

In another embodiment, when the ISVD specifically binding to human CD8alpha has a sequence identity of more than 90%, such as more than 95% or more than 99%, with one of the ISVDs with SEQ ID NOs: 160-169, the ISVD has at least (essentially) the same binding affinity to human CD8alpha compared to one of the ISVDs with SEQ ID NOs: 160-169, wherein the binding affinity is measured using the same method, such as e.g. SPR.

In another embodiment, when the ISVD specifically binding to human CD8alpha has a sequence identity of more than 90%, such as more than 95% or more than 99%, with one of the ISVDs with SEQ ID NOs: 169, the ISVD has at least (essentially) the same binding affinity to cyno CD8alpha compared to one of the ISVDs with SEQ ID NOs: 160-169, wherein the binding affinity is measured using the same method, such as e.g. SPR.

In another embodiment, when the ISVD specifically binding to human CD8alpha has a sequence identity of more than 90%, such as more than 95% or more than 99%, with one of the ISVDs with SEQ ID NOs: 160-169, the ISVD still specifically binds to human CD8alpha and cyno CD8alpha with less than 10-fold difference in affinity (K_{D}).

In another embodiment, when the ISVD specifically binding to human CD8alpha has a sequence identity of more than 90%, such as more than 95% or more than 99%, with one of the ISVDs with SEQ ID NOs: 160-169, the ISVD has at least (essentially) half of the potency, preferably at least (essentially) the same potency for binding to CD8+ T cells compared to one of the ISVDs with SEQ ID NOs: 160-169, wherein the potency is monitored via FACS binding assay e.g., the FACS assay on human T cells (purified from peripheral blood monocytes) as described in the examples.

In some embodiments, the immunoglobulin single variable domains of the present technology specifically bind to human CD8alpha with a dissociation constant (KD) of 10⁻⁷ to 10⁻¹¹ moles/litre or less, and preferably 1×10⁻⁸ to 10⁻¹⁰ moles/litre or less such as more preferably 10⁻⁹ to 10⁻¹⁰ moles/litre, even more preferably wherein the ISVD specifically binds to human CD8alpha with a K_{D} of about 1.33×10⁻¹⁰ moles/litre, as determined by SPR.

In some embodiments, the immunoglobulin single variable domains of the present technology specifically bind to cyno CD8alpha with a dissociation constant (KD) of 10⁻⁷ to 10⁻¹¹ moles/litre or less, and preferably 1×10⁻⁷ to 10⁻¹⁰ moles/litre or less such as more preferably 10⁻⁸ to 10⁻¹⁰ moles/litre, even more preferably wherein the ISVD specifically binds to cyno CD8alpha with a K_{D} of about 1.06×10⁻¹⁰ moles/litre, as determined by SPR.

In some embodiments, the immunoglobulin single variable domains of the present technology specifically bind to human CD8alpha and cyno CD8alpha with less than 10-fold difference in affinity (KD).

A preferred assay for measuring binding of the ISVD (or polypeptide comprising the ISVD) to CD8alpha exposed on a cell surface is a FACS assay, such as e.g. the FACS assay as described in the examples, wherein binding of the ISVDs or polypeptides to CD8alpha on CD8⁺ T cells is determined.

In some embodiments, the immunoglobulin single variable domains of the present technology specifically bind to human CD8alpha. For example, in FACS binding assay on CD8⁺ T cells, the immunoglobulin single variable domains of the present technology may have EC50 values of 10⁻⁷ M or lower, more preferably of 10⁻⁸ M or lower. For example, in such FACS binding assay, the immunoglobulin single variable domains of the present technology may have EC50 of 10⁻⁷ to 10⁻¹¹ M or less, and preferably 1×10⁻⁷ to 10⁻¹⁰ M or less such as more preferably 1×10⁻⁸ to 1×10⁻¹⁰ M, even more preferably wherein the ISVD specifically binds to human CD8alpha on CD8⁺ T cells with a EC50 of about 2.0×10⁻¹⁰M, as determined by FACS assay on CD8⁺ T cells.

In some embodiments, the ISVDs as described herein may further comprise additional amino acids at either the N-terminus and/or the C-terminus. In some embodiments, the additional amino acids are at the C-terminus. In some embodiments, the additional amino acids form a tag peptide that can facilitate purification. In some embodiments, the tag peptide is a poly-histidine (such as 6×His). In some embodiments, the additional amino acids can form a tag for downstream modification of the ISVD, such as a tag for conjugation reaction. In some embodiments, the tag for conjugation reaction comprises at least one cysteine, which can help with a downstream cysteine-based click chemistry reaction. In some embodiments, the tag for click chemistry reaction comprises GGC. In some embodiments, the additional amino acid can form both a tag for click chemistry, and a tag that can facilitate purification. In some embodiments, the tag for click chemistry reaction is GGC. For example, GGC can be added to the C-terminus of an ISVD (e.g. an anti-CD8α ISVD) as described herein, such as any ISVD of SEQ ID Nos: 160 to 179, to form new ISVD of SEQ ID Nos: 28-36 and 44. In some embodiments, the phospholipid-PEG-anti-CD8α conjugate is DSPE-PEG3.4K-A044300805_v8_GGC (a.k.a., T0347015C01v8, SEQ ID NO: 44).

In some embodiments, the ISVDs as described herein comprise at last one internal disulfide bridge. In some embodiments, the ISVDs as described herein comprise two internal disulfide bridge, such as a canonical disulfide bridge and a VHH-specific bridge (e.g., a VHH1 specific disulfide bridge). In some embodiments, the ISVD is a VHH1-type ISVD. In some embodiments, the VHH1-type ISVD has two internal disulfide bridges (e.g., 1×canonical+1× exposed VHH1-specific), and one free cysteine for conjugation at the C-terminus (e.g., a GGC linker).

In the ISVDs of the disclosure that comprise the combinations of CDRs mentioned above, each CDR can be replaced by a CDR chosen from the group consisting of amino acid sequences that have at least 80%, preferably at least 90%, more preferably at least 95%, even more preferably at least 99% sequence identity with the mentioned CDRs; in which:
(1) any amino acid substitution is preferably a conservative amino acid substitution; and/or
(2) said amino acid sequence preferably only contains amino acid substitutions, and no amino acid deletions or insertions, compared to the above amino acid sequence(s);
and/or chosen from the group consisting of amino acid sequences that have 3, 2 or only 1 (as indicated in the preceding paragraph) “amino acid difference(s)” with the mentioned CDR(s) of one of the above amino acid sequences, in which:
(1) any amino acid substitution is preferably a conservative amino acid substitution; and/or
(2) said amino acid sequence preferably only contains amino acid substitutions, and no amino acid deletions or insertions, compared to the above amino acid sequence(s).

In one embodiment, the anti-CDα 8 ISVD is BDSn:

### Anti-CD8 BDSn Nb Sequence (CDR1, CDR2, CDR3 Underlined Based on IMGT Designation):

(SEQ ID NO: 419)
EVQLVESGGGLVQAGGSLRLSCAASGSTFSDYGVGWFRQAPGKGREFVA
DIDWNGEHTSYADSVKGRFATSRDNAKNTAYLQMNSLKPEDTAVYYCAA
DALPYTVRKYNYWGQGTQVTVSSGGCGGHHHHHH

In some embodiments, the anti-CD8α ISVD in a phospholipid-PEG-antibody conjugate is derived from SEQ ID NO: 419, such as listed in the Table C-4 below.

TABLE C-4
Anti-CD8 BDSn modified ISVDs

| ISVD name | | |
|---|---|---|
| SES ID NO | Description | Sequence |
| A044300694 | CD8aBDSn(L11V, A14P, N53E, A68T, T69I, | EVQLVESGGGVVQPGGSLRLSCAASGSTESDYGVG |
| SEQ ID NO: 10 | K83R, V89L, N101R, Q108L) | WFRQAPGKGREFVADIDWEGEHTSYADSVKGRFTI |
| | | SRDNAKNTAYLQMNSLRPEDTALYYCAADALPYT |
| | | VRKYRYWGQGTLVTVSS |
| A044300694_v1 | CD8aBDSn(L11V, A14P, N53E, A68T, T69I, | EVQLVESGGGVVQPGGSLRLSCAASGSTFSDYGVG |
| SEQ ID NO: 11 | K83R, V89L, A94E, N10IR, Q108L) | WFRQAPGKGREFVADIDWEGEHTSYADSVKGRFTI |
| | | SRDNAKNTAYLQMNSLRPEDTALYYCAEDALPYT |
| | | VRKYRYWGQGTLVTVSS |
| A044300694_v2 | CD8aBDSn(L11V, A14P, N53E, A68T, T69I, | EVQLVESGGGVVQPGGSLRLSCAASGSTFSDYGVG |
| SEQ ID NO: 12 | K83R, V89L, A94Y, N101R, Q108L) | WFRQAPGKGREFVADIDWEGEHTSYADSVKGRFTI |
| | | SRDNAKNTAYLQMNSLRPEDTALYYCAYDALPYT |
| | | VRKYRYWGQGTLVTVSS |
| A044300694_v3 | CD8aBDSn(L11V, A14P, N53E, A68T, T69I, | EVQLVESGGGVVQPGGSLRLSCAASGSTFSDYGVG |
| SEQ ID NO: 13 | K83R, V89L, V100aL, N101R, Q108L) | WFRQAPGKGREFVADIDWEGEHTSYADSVKGRFTI |
| | | SRDNAKNTAYLQMNSLRPEDTALYYCAADALPYT |
| | | LRKYRYWGQGTLVTVSS |
| A044300694_v4 | CD8aBDSn(L11V, A14P, N53E, A68T, T69I, | EVQLVESGGGVVQPGGSLRLSCAASGSTFSDYGVG |
| SEQ ID NO: 14 | K83R, V89L, A94E, V100aL, N10IR, Q108L) | WFRQAPGKGREFVADIDWEGEHTSYADSVKGRFTI |
| | | SRDNAKNTAYLQMNSLRPEDTALYYCAEDALPYT |
| | | LRKYRYWGQGTLVTVSS |
| A044300694_v5 | CD8aBDSn(L11V, A14P, N53E, A68T, T69I, | EVQLVESGGGVVQPGGSLRLSCAASGSTFSDYGVG |
| SEQ ID NO: 15 | K83R, V89L, A94Y, V100aL, N101R, Q108L) | WFRQAPGKGREFVADIDWEGEHTSYADSVKGRFTI |
| | | SRDNAKNTAYLQMNSLRPEDTALYYCAYDALPYT |
| | | LRKYRYWGQGTLVTVSS |
| A044300694_v6 | CD8aBDSn(L11V, A14P, N53E, A68T, T69I, | EVQLVESGGGVVQPGGSLRLSCAASGSTFSDYGVG |
| SEQ ID NO: 16 | Y79D, K83R, V89L, N101R, Q108L) | WFRQAPGKGREFVADIDWEGEHTSYADSVKGRFTI |
| | | SRDNAKNTADLQMNSLRPEDTALYYCAADALPYT |
| | | VRKYRYWGQGTLVTVSS |
| A044300694_v7 | CD8aBDSn(L11V, A14P, N53E, A68T, T69I, | EVQLVESGGGVVQPGGSLRLSCAASGSTFSDYGVG |
| SEQ ID NO: 17 | Y79D, K83R, V89L, V100aL, N101R, Q108L) | WFRQAPGKGREFVADIDWEGEHTSYADSVKGRFTI |
| | | SRDNAKNTADLQMNSLRPEDTALYYCAADALPYT |
| | | LRKYRYWGQGTLVTVSS |
| A044300694_v8 | CD8aBDSn(L11V, A14P, N53E, A68T, T69I, | EVQLVESGGGVVQPGGSLRLSCAASGSTFSDYGVG |
| SEQ ID NO: 18 | Y79D, K83R, V89L, A94E, V100aL, N101R, | WFRQAPGKGREFVADIDWEGEHTSYADSVKQRFTI |
| | Q108L) | SRDNAKNTADLQMNSLRPEDTALYYCAEDALPYT |
| | | LRKYRYWGQGTLVTVSS |
| A044300694_v9 | CD8aBDSn(L11V, A14P, N53E, A68T, T69I, | EVQLVESGGGVVQPGGSLRLSCAASGSTFSDYGVG |
| SEQ ID NO: 19 | Y79D, K83R, V89L, A94Y, V100aL, N101R, | WFRQAPGKGREFVADIDWEGEHTSYADSVKGRFTI |
| | Q108L) | SRDNAKNTADLQMNSLRPEDTALYYCAYDALPYT |
| | | LRKYRYWGQGTLVTVSS |
| A044300694_v10 | CD8aBDSn(L11V, A14P, N53E, A68T, T69I, | EVQLVESGGGVVQPGGSLRLSCAASGSTFSDYGVG |
| SEQ ID NO: 20 | Y79D, K83R, V89L, A94E, N101R, Q108L) | WFRQAPGKGREFVADIDWEGEHTSYADSVKGRFTI |
| | | SRDNAKNTADLQMNSLRPEDTALYYCAEDALPYT |
| | | VRKYRYWGQGTLVTVSS |
| A044300694_v11 | CD8aBDSn(L11V, A14P, N53E, A68T, T69I, | EVQLVESGGGVVQPGGSLRLSCAASGSTFSDYGVG |
| SEQ ID NO: 21 | Y79D, K83R, V89L, A94Y, N101R, Q108L) | WFRQAPGKGREFVADIDWEGEHTSYADSVKGRFTI |
| | | SRDNAKNTADLQMNSLRPEDTALYYCAYDALPYT |
| | | VRKYRYWGQGTLVTVSS |
| A044300694_v12 | CD8aBDSn(L11V, A14P, N53E, A68T, T69I, | EVQLVESGGGVVQPGGSLRLSCAASGSTFSDYGVG |
| SEQ ID NO: 22 | Y79S, K83R, V89L, V100aL, N101R, | WFRQAPGKGREFVADIDWEGEHTSYADSVKGRFTI |
| | Q108L) | SRDNAKNTASLQMNSLRPEDTALYYCAADALPYT |
| | | LRKYRYWGQGTLVTVSS |
| A044300694_v13 | CD8aBDSn(L11V, A14P, N53E, A68T, T69I, | EVQLVESGGGVVQPGGSLRLSCAASGSTFSDYGVG |
| SEQ ID NO: 23 | Y79S, K83R, V89L, A94E, V100aL, N101R, | WFRQAPGKGREFVADIDWEGEHTSYADSVKGRFTI |
| | Q108L) | SRDNAKNTASLQMNSLRPEDTALYYCAEDALPYTL |
| | | RKYRYWGQGTLVTVSS |
| A044300694_v14 | CD8aBDSn(L11V, A14P, N53E, A68T, T69I, | EVQLVESGGGVVQPGGSLRLSCAASGSTFSDYGVG |
| SEQ ID NO: 24 | Y798, K83R, V89L, A94Y, V100aL, N101R, | WFRQAPGKGREFVADIDWEGEHTSYADSVKGRFTI |
| | Q108L) | SRDNAKNTASLQMNSLRPEDTALYYCAYDALPYT |
| | | LRKYRYWGQGTLVTVSS |
| A044300694_v15 | CD8aBDSn(L11V, A14P, N53E, A68T, T69I, | EVQLVESGGGVVQPGGSLRLSCAASGSTFSDYGVG |
| SEQ ID NO: 25 | Y79S, K83R, V89L, N101R, Q108L) | WFRQAPGKGREFVADIDWEGEHTSYADSVKGRFTI |
| | | SRDNAKNTASLQMNSLRPEDTALYYCAADALPYT |
| | | VRKYRYWGQGTLVTVSS |
| A044300694_v16 | CD8aBDSn(L11V, A14P, N53E, A68T, T69I, | EVQLVESGGGVVQPGGSLRLSCAASGSTFSDYGVG |
| SEQ ID NO: 26 | Y79S, K83R, V89L, A94E, N101R, Q108L) | WFRQAPGKGREFVADIDWEGEHTSYADSVKGRFTI |
| | | SRDNAKNTASLQMNSLRPEDTALYYCAEDALPYT |
| | | VRKYRYWGQGTLVTVSS |
| A044300694_v17 | CD8aBDSn(L11V, A14P, N53E, A68T, T69I, | EVQLVESGGGVVQPGGSLRLSCAASGSTFSDYGVG |
| SEQ ID NO: 27 | Y79S, K83R, V89L, A94Y, N101R ,Q108L) | WFRQAPGKGREFVADIDWEGEHTSYADSVKGRFTI |
| | | SRDNAENTASLQMNSLRPEDTALYYCAYDALPYT |
| | | VRKYRYWGQGTLVTVSS |

In some embodiments, the anti-CD8α ISVD in a phospholipid-PEG-antibody conjugate is T0347015C01, A044300805, A044300805_v1, A044300805_v2, A044300805_v3, A044300805_v4, A044300805_v5, A044300805_v6, A044300805_v7, or A044300805_v8 (e.g., SEQ ID NOs: 160 to 169), or a modified version wherein a polypeptide comprising cysteine (e.g., GGC) is added to the C-terminus of each (e.g., SEQ ID NOs: 28-36 and 44). In some embodiments, the anti-CD8α ISVD in the phospholipid-PEG-antibody conjugate is SEQ ID NO: 44.

In some embodiments, an anti-CD8α ISVD of the present disclosure binds to CD8 with an dissociation constant (KD) of 10⁻⁵ to 10⁻¹² moles/liter (M) or less, and preferably 10⁻⁷ to 10⁻¹² moles/liter (M) or less and more preferably 10⁻⁸ to 10⁻¹² moles/liter (M), and/or with an association constant (KA) of at least 10⁷ M⁻¹, preferably at least 10⁸ M⁻¹, more preferably at least 10⁹ M⁻¹ such as at least 10¹² M⁻¹; and in particular with a KD less than 500 nM, preferably less than 200 nM, more preferably less than 10 nM, such as less than 500 μM. The KD and KA values of the ISVD of the disclosure against CD8 can be determined in a manner known per se, for example using the assay described herein. More generally, the ISVDs described herein preferably have a dissociation constant with respect to CD8 that is as described in this paragraph.

Generally, it should be noted that the term ISVD as used herein in its broadest sense is not limited to a specific biological source or to a specific method of preparation. For example, as will be discussed in more detail below, the ISVD can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding a such humanized VHH domain; (4) by “camelization” (as described below) of a naturally occurring VH domain from any animal species, in particular a species of mammal, such as from a human being, or by expression of a nucleic acid encoding such a camelized VH domain; (5) by “camelisation” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a VHH domain, VH domain and/or dAb using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the anti-CD8 ISVDs of the present disclosure do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

One class of anti-CD8α ISVDs of the disclosure comprises ISVDs with an amino acid sequence that corresponds to the amino acid sequence of a naturally occurring VHH domain, but that has been “humanized”, i.e. by replacing one or more amino acid residues in the amino acid sequence of said naturally occurring VHH sequence by one or more of the amino acid residues that occur at the corresponding position(s) in a VH domain from a conventional 4-chain antibody from a human being (e.g., indicated above). It should be noted that such humanized anti-CD8α ISVDs of the present disclosure can be obtained in any suitable manner known per se (i.e. as indicated under points (1)-(8) above) and thus are not strictly limited to polypeptides that have been obtained using a polypeptide that comprises a naturally occurring VHH domain as a starting material.

Another class of anti-CD8α ISVDs of the present disclosure comprises ISVDs with an amino acid sequence that corresponds to the amino acid sequence of a naturally occurring VH domain that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description below. Reference is also made to WO 94/04678. Such camelization may preferentially occur at amino acid positions which are present at the VH-VL interface and at the so-called Camelidae hallmark residues (see for example also WO 94/04678), as also mentioned below. In some embodiments, the VH domain or sequence that is used as a starting material or starting point for generating or designing the camelized ISVD is a VH sequence from a mammal, e.g., VH sequence of a human being. It should be noted that such camelized ISVD of the present disclosure can be obtained in any suitable manner known per se and thus are not strictly limited to polypeptides that have been obtained using a polypeptide that comprises a naturally occurring VH domain as a starting material.

Other anti-CD8 antibodies can also be used, such as those disclosed in WO2025129201A1, which is incorporated by reference in its entirety for all purposes.

For example, both “humanization” and “camelization” can be performed by providing a nucleotide sequence that encodes such a naturally occurring VHH domain or VH domain, respectively, and then changing, in a manner known per se, one or more codons in said nucleotide sequence such that the new nucleotide sequence encodes a humanized or camelized ISVD of the present disclosure, respectively, and then expressing the nucleotide sequence thus obtained in a manner known per se so as to provide the desired ISVD. Alternatively, based on the amino acid sequence of a naturally occurring VHH domain or VH domain, respectively, the amino acid sequence of the desired humanized or camelized ISVD of the present disclosure, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized ISVD can be designed and then synthesized de novo using techniques for nucleic acid synthesis known per se, after which the nucleotide sequence thus obtained can be expressed in a manner known per se so as to provide the desired ISVD.

Other suitable ways and techniques for obtaining ISVDs and/or nucleotide sequences and/or nucleic acids encoding the same, starting from (the amino acid sequence of) naturally occurring VH domains or preferably VHH domains and/or from nucleotide sequences and/or nucleic acid sequences encoding the same will be clear from the skilled person, and may for example comprising combining one or more amino acid sequences and/or nucleotide sequences from naturally occurring VH domains (such as one or more FR's and/or CDR's) with one or more one or more amino acid sequences and/or nucleotide sequences from naturally occurring VHH domains (such an one or more FR's or CDR's), in a suitable manner so as to provide (a nucleotide sequence or nucleic acid encoding) an ISVD. Also provided are compounds and constructs, and in particular proteins and polypeptides that comprise or essentially consists of at least one such amino acid sequence and/or ISVD of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or ISVD (and/or to the compound or construct in which it is present) and may or may not modify the properties of the amino acid sequence and/or ISVD.

The disclosure also encompasses any polypeptide of the present disclosure that has been glycosylated at one or more amino acid positions, usually depending on the host used to express the polypeptide. A polypeptide can comprise an amino acid sequence of an anti-CD8α ISVD of the present disclosure, which is fused at its amino terminal end, at its carboxy terminal end, or both at its amino terminal end and at its carboxy terminal end with at least one further amino acid sequence. Such further amino acid sequence may comprise at least one further ISVD, so as to provide a polypeptide that comprises at least two, such as three, four or five, ISVDs, in which said ISVDs may optionally be linked via one or more linker sequences (as defined herein). Polypeptides of comprising the anti-CD8α ISVD of the present disclosure and one or more other ISVDs are multivalent polypeptides. In a multivalent polypeptide, the two or more ISVDs may be the same or different. For example, the two or more ISVDs in a multivalent polypeptide:
may be directed against the same antigen, i.e. against the same parts or epitopes of said antigen or against two or more different parts or epitopes of said antigen; and/or:
may be directed against the different antigens;
or a combination thereof.

Thus, a bivalent polypeptide, for example:
may comprise two identical ISVDs;
may comprise a first ISVD directed against a first part or epitope of an antigen and a second ISVD directed against the same part or epitope of said antigen or against another part or epitope of said antigen;
or may comprise a first ISVD directed against a first antigen and a second ISVD directed against a second antigen different from said first antigen;
whereas a trivalent polypeptide of the present disclosure for example:
may comprise three identical or different ISVDs directed against the same or different parts or epitopes of the same antigen;
may comprise two identical or different ISVDs directed against the same or different parts or epitopes on a first antigen and a third ISVD directed against a second antigen different from said first antigen; or
may comprise a first ISVD directed against a first antigen, a second ISVD directed against a second antigen different from said first antigen, and a third ISVD directed against a third antigen different from said first and second antigen.

The anti-CD8α ISVDs and polypeptides as disclosed herein can also be introduced and expressed in one or more cells, tissues or organs of a multicellular organism, for example for prophylactic and/or therapeutic purposes (e.g., as a gene therapy). For this purpose, the nucleotide sequences encoding the anti-CD8α ISVDs or polypeptides as disclosed herein can be introduced into the cells or tissues in any suitable way, for example as such (e.g., using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patient by administering a nucleic acid of the present disclosure or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the present disclosure and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. Nos. 5,580,859; 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

The disclosure also encompasses compositions comprising at least one ISVD or at least one polypeptide or construct of the present technology, at least one nucleic acid molecule encoding a polypeptide of the present technology or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

Accordingly, nucleic acid sequences encoding the anti-CD8α ISVDs as described herein, and expression construct and host cells comprising the nucleic acid sequence are also provided. Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

The present technology also provides a method for producing the ISVD and/or polypeptide of the present technology. The method may comprise transforming/transfecting a (non-human) host cell or host organism with a nucleic acid encoding the ISVD and/or polypeptide, expressing the ISVD and/or polypeptide in the host, optionally followed by one or more isolation and/or purification steps. In an embodiment, the method may comprise:
a) expressing, in a suitable (non-human) host cell or host organism or in another suitable expression system, a nucleic acid sequence or genetic construct encoding the ISVD and/or polypeptide; optionally followed by:
b) isolating and/or purifying the ISVD and/or polypeptide.

In another embodiment, the method may comprise:
a) cultivating and/or maintaining a (non-human) host or host cell that is capable of expressing the ISVD and/or polypeptide of the present technology and/or that comprises a nucleic acid or a genetic construct encoding the ISVD and/or polypeptide of the present technology, under suitable circumstances that are such that said (non-human) host or host cell is expresses the ISVD and/or polypeptide of the present technology;
optionally followed by:
b) isolating and/or purifying the ISVD and/or polypeptide produced.

Suitable (non-human) host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

Also disclosed are methods of using anti-CD8α ISVDs and polypeptides of the present disclosure.

In some embodiments, a polypeptide comprising an anti-CD8α ISVD can be used in the lipid nanoparticles of the present disclosure for delivering a nucleic acid into an immune cell, as described herein. In some embodiments, anti-CD8α ISVDs and polypeptides of the present disclosure can be used to treat a condition or a disease in a subject in need thereof. In some embodiments, such conditions or diseases include, but are not limited to, cancer, infections, immune disorders, autoimmune diseases.

Also provided is the ISVD of the present disclosure, the polypeptide or construct comprising the ISVD, the nucleic acid molecule or vector as described, or the composition comprising the ISVD, polypeptide, construct, nucleic acid molecule or vector of the present disclosure, for use in the diagnosis, prevention and/or treatment of at least one disease and/or disorder that is associated with CD8α, with its biological or pharmacological activity, and/or with the biological pathways or signaling in which CD8α is involved.

Also provided is the ISVD of the present disclosure, the polypeptide or construct comprising the ISVD, the nucleic acid molecule or vector as described, or the composition comprising the ISVD, polypeptide, construct, nucleic acid molecule or vector of the present disclosure for use in the diagnosis, prevention and/or treatment of a proliferative disease (such as cancer), an inflammatory disease, and/or an infectious disease. Therefore, also provided is the ISVD of the present disclosure, the polypeptide or construct of the present disclosure, the nucleic acid molecule or vector as described, or the composition comprising the ISVD, polypeptide, construct, nucleic acid molecule or vector of the present disclosure for use in the diagnosis, prevention and/or treatment of a proliferative disease (such as cancer), an inflammatory disease, and/or an infectious disease.

In some embodiments, a polypeptide comprising an anti-CD8α ISVD can be used in an imaging agent. In some embodiments, the imaging agent allows for the detection of human CD8 which is a specific biomarker found on the surface of a subset of T-cell for diagnostic imaging of the immune system. Imaging of CD8 allows for the in vivo detection of T-cell localization. Changes in T-cell localization can reflect the progression of an immune response and can occur over time as a result of various therapeutic treatments or even disease states. In some embodiments, it is used for imaging T-cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCI protein of antigen presenting cells. In some embodiments, a polypeptide comprising an anti-CD8α ISVD can potentiate signaling through the T cell receptor and enhance the ability of a subject to clear viral pathogens and respond to tumor antigens. Thus, in some embodiments, the antigen binding constructs provided herein can be agonists and can activate the CD8 target.

General methods for purifying ISVD polypeptides (such as VHs and VHHs) can be used to produce ISVD that is used to make phospholipid-PEG-ISVD (e.g., VHH) conjugate, such as those described in WO 2010/125187 and WO 2012/056000.

### III. Lipid Nanoparticles (LNPs) and Compositions

In one aspect, provided is a lipid nanoparticle (LNP) or LNP composition. In some embodiments, the LNP or LNP composition comprises (i) a lipid-immune cell targeting group conjugate.

In some embodiments, the LNPs also comprise (ii) an ionizable cationic lipid.

In some embodiments, the LNPs further comprise (iii) a structural lipid.

In some embodiments, the LNP further comprise (iv) a neutral phospholipid.

In some embodiments, the LNPs further comprise (v) a free PEG-lipid.

In some embodiments, the LNPs further comprise (vi) a payload.

In some embodiments, the LNPs comprises (i) to (vi) and any combination thereof.

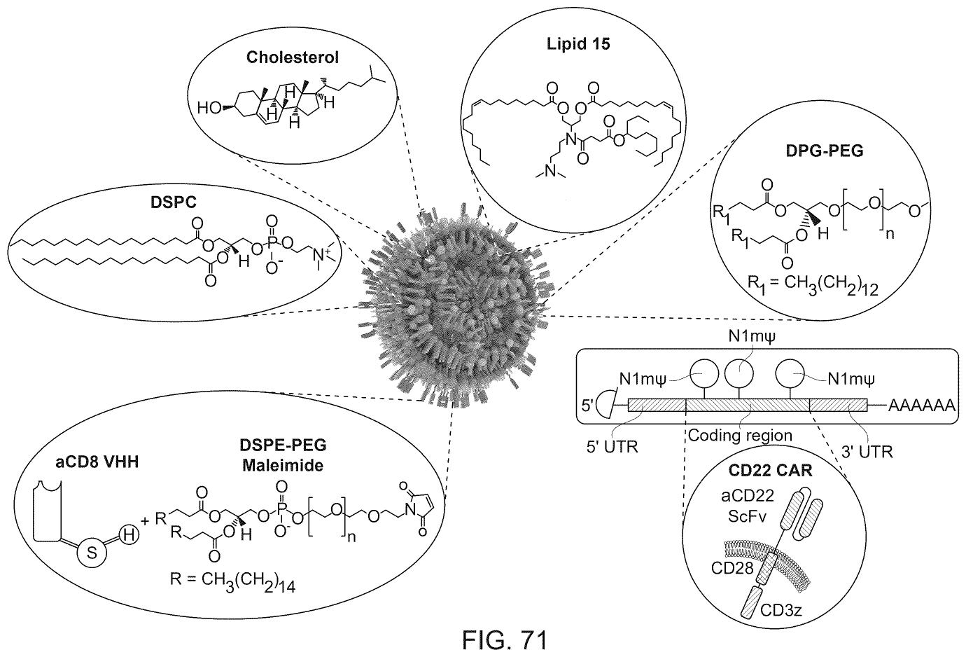

In some embodiments, the LNPs comprise a formulation as illustrated in FIG. 71.

In some embodiments, the LNP or LNP composition comprises DSPE-PEG 3.4K-anti-CD8α ISVD conjugate (lipid-immune cell targeting group conjugate), Lipid 15 (ionizable cationic lipid), mRNA (payload), cholesterol (structural lipid), DSPC (neutral phospholipid), and PEG 2000-DPG (same as DPG-PEG 2K; free PEG-lipid). In some embodiments, the mRNA encodes a chimeric antigen receptor (CAR) comprises an anti-CD22 antibody, such as an anti-CD22 Single-chain variable fragment (ScFv). Non-limiting examples of each element are described herein.

In some embodiments, the LNP or LNP composition further comprises one or more additional components. For example, it may further comprise one or more additional components that are included in the LNP due to a manufacturing process that is used to produce the LNP. For example, as described herein, to produce a phospholipid-PEG-antibody conjugate, a phospholipid-PEG that does not have a bioconjugation linker may be included with the LNP or the LNP composition. In some embodiments, such phospholipid-PEG not having a bioconjugation linker is DSPE-PEG. In some embodiments, the DSPE-PEG has a PEG with a molecular weight smaller than the PEG in the phospholipid-PEG-antibody conjugate. In some embodiments, the DSPE-PEG has a PEG with a molecular weight of about 2.0 kDa, and the PEG in the phospholipid-PEG-antibody conjugate has a molecular weight of about 3.4 kDa.

In some embodiments, the DSPE-PEG2.0k has a concentration of less than about 0.1 mol %, 0.09 mol %, 0.08 mol %, 0.07 mol %, 0.06 mol %, 0.05 mol %, 0.04 mol %, 0.03 mol %, 0.02 mol %, 0.01 mol %, 0.009 mol %, 0.008 mol %, 0.007 mol %, 0.006 mol %, 0.005 mol %, 0.004 mol %, 0.003 mol %, 0.002 mol %, or 0.001 mol % in the LNP, or a composition comprising the LNP, excluding solvent.

In some embodiments, the DSPE-PEG2.0k has a concentration of less than about 0.1 mol %, 0.09 mol %, 0.08 mol %, 0.07 mol %, 0.06 mol %, 0.05 mol %, 0.04 mol %, 0.03 mol %, 0.02 mol %, 0.01 mol %, 0.009 mol %, 0.008 mol %, 0.007 mol %, 0.006 mol %, 0.005 mol %, 0.004 mol %, 0.003 mol %, 0.002 mol %, or 0.001 mol % in the LNP, or a composition comprising the LNP, excluding solvent. In some embodiments, the DSPE-PEG2.0k has a concentration of between about 0.01 mol % and about 0.02 mol % or between about 0.04 mol % and about 0.08 mol %, in the LNP, or a composition comprising the LNP, excluding solvent.

### A. Lipid-Immune Cell Targeting Group Conjugates

In some embodiments, the lipid-immune cell targeting group conjugate comprises the compound of Formula (II): [Lipid]-[optional linker]-[antibody]. In some embodiments, the Lipid of Formula (II) is a phospholipid. In some embodiments, the optional linker of Formula (II) is PEG. In some embodiments, the lipid-immune cell targeting group conjugate comprises DSPE-PEG 3.4K-anti CD8 antibody conjugate. In some embodiments, the conjugate is produced from conjugating an anti-CD8α immunoglobulin single variable domain (ISVD, such as a VHH) such as those described in the “II. Immunoglobulin single variable domain” section above, and DSPE-PEG having a bioconjugation linker, such as maleimide. In some embodiments, the DSPE-PEG maleimide is DSPE-PEG3.4K-maleimide. In some embodiments, the anti-CD8α ISVD in the conjugate comprises any one of SEQ ID NOs: 160 to 169 or any one of SEQ ID NOs 28-36 and 44, such as SEQ ID NO: 44, or any one of SEQ ID NOs: 10 to 27.

In some embodiments, the antibody of Formula (II) comprises an ISVD that can bind to and/or are directed against CD8α (CD8alpha) and that comprise CDR sequences that are generally as further defined herein, to suitable fragments thereof, as well as to polypeptides that comprise or essentially consist of one or more of such ISVDs and/or suitable fragments. In some embodiments, the antibody of Formula (II) comprises an ISVD comprising a sequence selected from the group consisting of SEQ ID NOs: 160 to 179. In some embodiments, the antibody of Formula (II) comprises
I) ISVD that is directed against CD8α and that has at least 80%, preferably at least 85%, such as 90% or 95% or more sequence identity with an ISVD comprising a sequence selected from the group consisting of SEQ ID NOs: 160 to 179; and/or
II) ISVD that cross-blocks the binding of the amino acid sequence selected from the group consisting of SEQ ID NOs: 160 to 179 to CD8α and/or that compete with at least the ISVD selected from the group consisting of SEQ ID NOs: 160 to 179 for binding to CD8α.

Such ISVDs, as part of Formula (II), may be as further described herein (and may for example be VHHs, including humanized VHHs, VHs, including human VHs, camelized VHs and camelized human VHs); as well as polypeptides of the disclosure that comprise one or more of such amino acid sequences (which may be as further described herein), and particularly bispecific (or multispecific) polypeptides as described herein, and nucleic acid sequences that encode ISVDs and polypeptides. Such ISVDs and polypeptides do not include any naturally occurring ligands.

In some embodiments, the CD8α is derived from a mammalian animal, such as a human being. In some embodiments, the antibody of Formula (II) comprises an ISVD directed against CD8α, that comprises:
a) the amino acid sequence selected from the group consisting of SEQ ID NOs: 160 to 179;
b) amino acid sequences that have at least 80% amino acid identity with a sequence selected from the group consisting of SEQ ID NOs: 160 to 179, or
c) amino acid sequences that have 3, 2, or 1 amino acid difference with a sequence selected from the group consisting of SEQ ID NOs: 160 to 179;
or any suitable combination thereof.

In some embodiments, the antibody of Formula (II) comprises an ISVD against CD8α, which consist of 4 framework regions (FR1 to FR4 respectively) and 3 complementarity determining regions (CDR1 to CDR3 respectively). In some embodiments, in such an ISVD:
(I) CDR1 comprises or essentially consists of an amino acid sequence selected from the group consisting of SEQ ID NOs: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition
or amino acid sequences that have at least 80%, at least 90%, at least 95%, at least 99% or more sequence identity with a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition;
and/or amino acids sequences that have 2 or only 1 amino acid difference(s) with a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition, in which
any amino acid substitution is a conservative amino acid substitution; and/or
said amino acid sequence only contains amino acid substitutions, and no amino acid deletions or insertions, compared to a sequence selected from the group consisting of SEQ ID NO: 181, 188, 195, 202, 209, 216, 223, 230, 237, and 244 according to Abm definition, and SEQ ID NOs: 251, 258, 265, 272, 279, 286, 293, 300, 307, and 314 according to Kabat definition.
(II) CDR2 comprises or essentially consists of an amino acid sequence comprising a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition,
or amino acid sequences that have at least 80%, at least 90%, at least 95%, at least 99% or more sequence identity with a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition;
and/or amino acids sequences that have 2 or only 1 amino acid difference(s) with a sequence selected from the group consisting of SEQ ID NO: 183, 190, 197, 204, 211, 218, 225, 232, 239 and 246 according to Abm definition, and SEQ ID NOs: 253, 260, 267, 274, 281, 288, 295, 302, 309 and 316 according to Kabat definition in which any amino acid substitution is a conservative amino acid substitution; and/or said amino acid sequence only contains amino acid substitutions, and no amino acid deletions or insertions, compared to a sequence selected from the group consisting of SEQ ID NO: 183, 190, 197, 204, 211, 218, 225, 232, 239 and 246 according to Abm definition and SEQ ID NOs: 253, 260, 267, 274, 281, 288, 295, 302, 309 and 316 according to Kabat definition.
(III) CDR3 comprises or essentially consists of an amino acid sequence comprising a sequence selected from the group consisting of SEQ ID NO: 185, 192, 199, 206, 213, 220, 227, 234, 241 and 248 according to Abm definition, and SEQ ID NOs: 255, 262, 269, 276, 283, 290, 297, 304, 311 and 318 according to Kabat definition,
or amino acid sequences that have at least 80%, at least 90%, at least 95%, at least 99% or more sequence identity with a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition;
and/or from the group consisting of amino acids sequences that have 2 or only 1 amino acid difference(s) with a sequence selected from the group consisting of SEQ ID NO: 185, 192, 199, 206, 213, 220, 227, 234, 241 and 248 according to Abm definition and SEQ ID NOs: 255, 262, 269, 276, 283, 290, 297, 304, 311 and 318 according to Kabat definition, in which
any amino acid substitution is a conservative amino acid substitution; and/or
said amino acid sequence only contains amino acid substitutions, and no amino acid deletions or insertions, compared to a sequence selected from the group consisting of SEQ ID NO: 185, 192, 199, 206, 213, 220, 227, 234, 241 and 248 according to Abm definition and SEQ ID NOs: 4 255, 262, 269, 276, 283, 290, 297, 304, 311 and 318 according to Kabat definition.

In one embodiment, the antibody of Formula (II) comprises ISVD that comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (according to Abm definition) or that comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (according to Kabat definition).

CD8 binding ISVDs as disclosed herein may comprise one, two or all three of the CDRs explicitly listed above. In some embodiments, the anti-CD8αISVD is selected from the ISVDs described in Table C-1, Table C-2, and Table C-3 above. In some embodiments, the anti-CD8αISVD is selected from the ISVDs that have at least 80%, at least 85%, at least 90%, at least 95%, at least 96%, at least 97%, at least 98%, or at least 99% sequence identity with the ISVDs described in Table C-1, Table C-2, and Table C-3 above. In some embodiments, the anti-CD8ISVD is selected from the ISVDs that have at least 80%, at least 85%, at least 90%, at least 95%, at least 96%, at least 97%, at least 98%, or at least 99% sequence identity with the ISVDs described in Table C-4 above.

In some embodiments, the lipid-immune cell targeting group conjugate is present in the LNP in a range of 0.001-0.5 mol percent, 0.001-0.1 mol %, 0.01-0.5 mol %, 0.05-0.5 mol %, 0.1-0.5 mol %, 0.1-0.3 mol %, 0.1-0.2 mol %, 0.2-0.3 mol %, of about 0.01 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, or about 0.5 mol %. In some embodiments, the lipid-immune cell targeting group conjugate is present in the LNP in a range of 0.01 to 0.03 mol percent. In some embodiment, the lipid-immune cell targeting group conjugate is present in the LNP in about 0.015 to about 0.016 mol percent.

In some embodiments, the lipid-immune cell targeting group conjugate is presented in the LNP at a density from about 0.4 to 11.4, about 1.9 to 9.5, about 3.8 to 7.6, about 4.6 to 6.5, about 5.3 to 6.1, about 3.8, or about 5.7 micromoles of conjugate per gram of mRNA in the LNP. In some embodiments, the lipid-immune cell targeting group conjugate comprises SEQ ID NO: 9, SEQ ID NO: 169, or SEQ ID NO: 44.

In one aspect, the present disclosure provides methods for producing the lipid-immune cell targeting group conjugate. In some embodiments, the lipid-immune cell targeting group conjugate comprises lipid-linker-antibody, such as a phospholipid-PEG-antibody. In some embodiments, the antibody is an ISVD. In some embodiments, the methods comprise (i) purifying the ISVD; and (ii) conjugating the ISVD to a phospholipid-PEG, such as a DSPE-PEG (e.g., DSPE-PEG3.4k-maleimide).

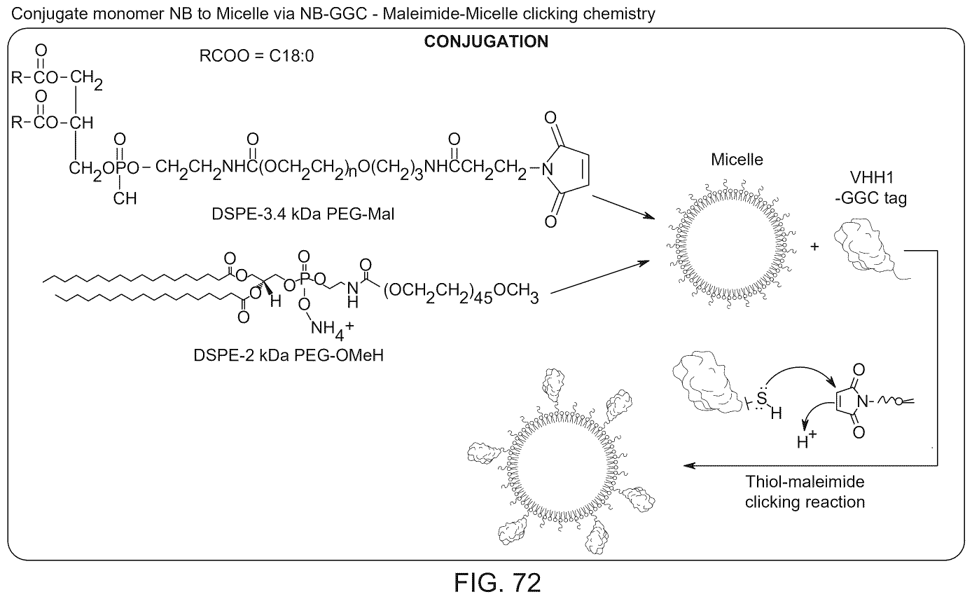

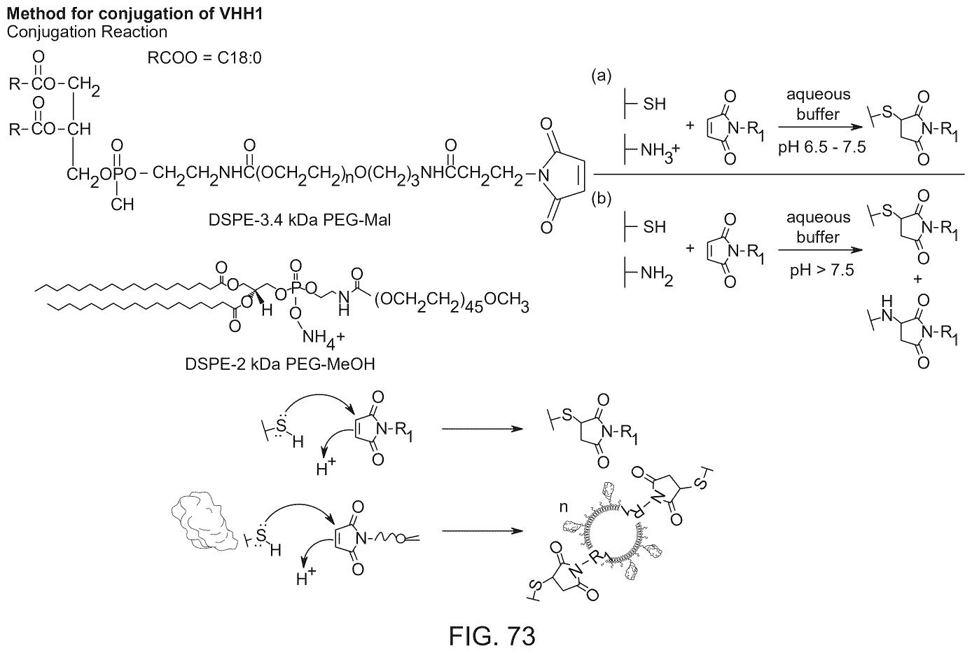

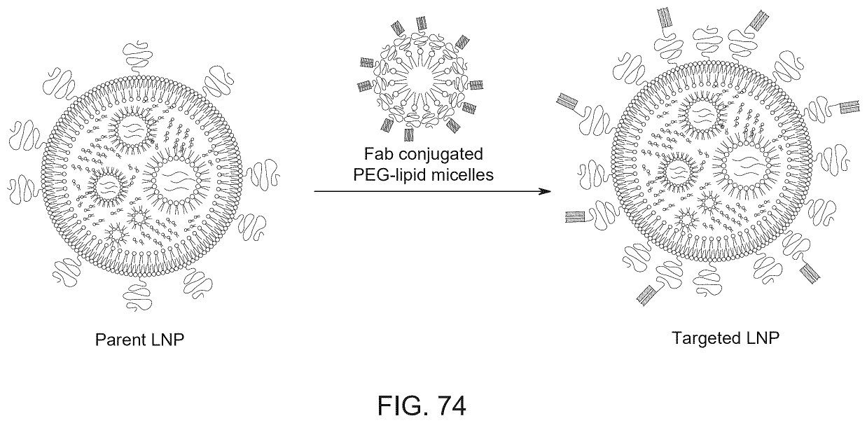

In some embodiments, non-limiting examples for the method of producing the conjugate is as illustrated in FIG. 72 and FIG. 73.

In some embodiments, after the production/expression of the polypeptide of an ISVD, the host cell proteins and other impurities can be removed from the culture medium by routine means. For example, the host cell proteins and other impurities can be removed by centrifugation or filtration. The solution obtained by removal of the host cell proteins and other impurities from the culture medium is also referred to as culture supernatant or clarified culture supernatant.

The ISVD mixture product can be purified from culture supernatant by methods described herein. In some embodiments, the methods include, but are not limited to chromatographic methods, including size exclusion chromatography (SEC), ion exchange chromatography (IEX), affinity chromatography (AC), hydrophobic interaction chromatography (HIC), mixed-mode chromatography (MMC). These methods can be performed alone or in combination with other purification methods, e.g., precipitation. Suitable combinations of purification methods for ISVDs and ISVD containing polypeptides can be chosen as needed.

Any or all chromatographic steps can be carried out by any suitable mechanical means. Chromatography may be carried out, for example, in a column. The column may be run with or without pressure and from top to bottom or bottom to top. The direction of the flow of fluid in the column may be reversed during the chromatography process. Chromatography may also be carried out using a batch process in which the solid media is separated from the liquid used to load, wash, and elute the sample by any suitable means, including gravity, centrifugation, or filtration.

Chromatography may also be carried out by contacting the sample with a filter that absorbs or retains some molecules in the sample more strongly than others. In the following description, the various embodiments are mostly described in the context of chromatography carried out in a column. It is understood, however, that use of a column is merely one of several chromatographic modalities that may be used, and the illustration using a column does not limit the application to column chromatography, as those skilled in the art may readily apply the teachings to other modalities as well, such as those using a batch process or filter.

Suitable supports may be any currently available or later developed materials having the characteristics necessary to practice the claimed method, and may be based on any synthetic, organic, or natural polymer. For example, commonly used support substances include organic materials such as cellulose, polystyrene, agarose, sepharose, polyacrylamide, polymethacrylate, dextran and starch, and inorganic materials, such as charcoal, silica (glass beads or sand) and ceramic materials. Suitable solid supports are disclosed, for example, in Zaborsky “Immobilized Enzymes” CRC Press, 1973, Table IV on pages 28-46.

General method conditions, solutions and/or buffers, as well as their concentration ranges for use in the different chromatographic processes can be determined based on standard handbooks on chromatography (see e.g. Günter Jagschies, Eva Lindskog (ed.) Biopharmaceutical Processing, Development, Design, and Implementation of Manufacturing Processes, 1st Ed. 2017, Elsevier).

The first step of an ISVD polypeptide purification process is often referred to as “the capture step”. The purpose of the capture step is to have a first reduction of process-related impurities (for example, but not limited to, host cell proteins (HCPs), color and DNA) and to capture the ISVD polypeptide product while maintaining a high recovery. In one embodiment, the capture step refers to the first purification step on protein A chromatography in bind and elute mode.

In one embodiment, the ISVD polypeptide containing preparations may be purified by Protein A chromatography. Staphylococcal Protein A (SpA) is a 42 kDa protein composed of five nearly homologous domains named as E, D, A, B and C in order from the N-terminus (Sjodhal Eur. J. Biochem. 78:471-490 (1977); Uhlen et al. J. Biol. Chem. 259:1695-1702 (1984)). These domains contain approximately 58 residues, each sharing about 65%-90% amino acid sequence identity. Binding studies between Protein A and antibodies have shown that while all five domains of SpA (E, D, A, B and C) bind to an IgG via its Fc region, domains D and E exhibit significant Fab binding (Ljungberg et al. Mol. Immunol. 30 (14): 1279-1285 (1993); Roben et al. J. Immunol. 154:6437-6445 (1995); Starovasnik et al. Protein Sei. 8:1423-1431 (1999). The Z-domain, a functional analogue and energy-minimized version of the B domain (Nilsson et al. Protein Eng. 1:107-113 (1987)), was shown to have negligible binding to the antibody variable domain region (Cedergren et al. Protein Eng. 6 (4): 441-448 (1993); Ljungberg et al. (1993) supra; Starovasnik et al. (1999) supra).

Until recently, commercially available Protein A stationary phases employed SpA (isolated from Staphylococcus aureus or expressed recombinantly) as their immobilized ligand. Using these columns, it has not been possible to use alkaline conditions for column regeneration and sanitation as is typically done with other modes of chromatography using non-proteinaceous ligands (Ghose et al. Biotechnology and Bioengineering Yol. 92 (6): 665-73 (2005)). A new resin (MabSELECT™ SuRe) has been developed to withstand stronger alkaline conditions (Ghose et al. (2005) supra). Using protein engineering techniques, a number of asparagine residues were replaced in the Z-domain of protein A and a new ligand was created as a tetramer of four identically modified Z-domains (Ghose et al. (2005) supra).

Accordingly, purification methods can be carried out using commercially available Protein A columns according to manufacturers' specification. For instance, MabSELECT™ columns or MabSELECT™ SuRe columns (GE Healthcare Products) can be used. MabSELECT™ is a commercially available resin containing recombinant SpA as its immobilized ligand. Other commercially available sources of Protein A column including, but not limited to, PROSEP-ATM (Millipore, U.K.), which consists of Protein A covalently coupled to controlled pore glass, can be usefully employed. Other useful Protein A formulations include Protein A Sepharose FAST FLOW™ (Amersham Biosciences, Piscataway, NJ), Amsphere™ A3 (JSR Life Sciences), and TOYOPEARL™ 650M Protein A (TosoHaas Co., Philadelphia, PA).

Protein purification by Protein A-based chromatography may be performed in a column containing an immobilized Protein A ligand (typically a column packed with modified support of methacrylate copolymer or agarose beads to which is affixed an adsorbent consisting of Protein A or functional derivatives thereof). The column is typically equilibrated with a buffer and a sample containing a mixture of proteins (the target protein, plus contaminating proteins) is loaded onto the column. As the mixture passes through the column, the target protein binds to the adsorbent (Protein A or derivative thereof) within the column, while some unbound impurities and contaminants flow through. Bound protein is then eluted from the column. In this process the target protein is bound to the column while impurities and contaminants flow through. Target protein is subsequently recovered from the eluate.

After host cell proteins are removed, a sample containing ISVD can be subjected to a process often referred to as “the polish step” which aims at purity improvement. For instance, a chromatography step (e.g., ion exchange chromatography) in bind and elute mode can be used to remove/reduce product related variants (e.g., but not limited to, High-molecular Weight (HMW) species, Low-Molecular Weight (LMW) species, and other charged variants) as well as some process related impurities (e.g., but not limited to, HCP, residual Protein A, DNA) still present after the capture step.

### Method for Purifying ISVD Monomers

Due to the free cysteine linker at the C-terminus of the ISVD molecules, ISVD harvested from host cells normally present as a mixture of both monomers and dimers (e.g., two ISVD molecules are bound together through S—S). For example, a typical fermentation process may lead to a mixture having over 60% ISVD dimers and less than 40% monomers. Accordingly, there is a need to isolate only ISVD monomers from the mixture before they are conjugated to the phospholipid-PEG.

Therefore, in one aspect, the present disclosure provides a method for preparing a composition comprising monomers of an ISVD with a cysteine containing linker at its C-terminal end from a mixture of monomers of the ISVD and dimers of the ISVD. In some embodiments, the method comprises (a) reducing ISVD dimers in the mixture to ISVD monomers with a first reducing agent. In some embodiments, the first reducing agent comprises TCEP (tris(2-carboxyethyl)phosphine). In some embodiments, the step (a) is conducted around 15 to 25° C., optionally around 20-22° C. In some embodiments, the first reducing agent comprises 20×TCEP. In some embodiments, the step (a) takes about 16 to 20 hours. Optionally, the mixture of monomers of the ISVD and dimers of the ISVD is subjected to a step of removing host cell proteins and DNA before being subjected to step (a). In some embodiments, Protein A chromatography is used to remove host cell proteins and DNA.

In some embodiments, the method further comprises (b) purifying the ISVD monomers obtained in step (a) to get a purified composition comprising the ISVD monomers. In some embodiments, the step (b) comprises using a chromatograph, such as an ion exchange chromatography (IEX).

In some embodiments, the method further comprises (c) reducing the purified composition obtained in step (b) with a second reducing agent. In some embodiments, the second reducing agent is as the same or different from the first reducing agent used in step (a). In some embodiments, the reducing agent also comprises TCEP. In some embodiments, the first reducing agent comprises 10×TCEP. In some embodiments, the step (c) is conducted around 15 to 25° C., optionally around 20-22° C. In some embodiments, the step (c) takes about 16 to 20 hours.

In some embodiments, the method further comprises (d) purifying the reduced composition obtained in step (c) to obtain a composition comprising monomers of the ISVD. In some embodiments, the step (d) comprises Ultrafiltration/Diafiltration (UF/DF). In some embodiments, the UF/DF membrane has a molecular weight cut-off of 10 kDa.

In some embodiments, at least 50%, at least 55%, at least 60%, at least 65%, at least 70%, at least 75%, at least 80%, at least 85%, at least 90%, at least 95% or more ISVD in the composition obtained from step (d) are in monomeric form.

### Method for Conjugating ISVD to Lipid or LNPs Containing Lipid

In one aspect, also provided is a method for preparation of a composition comprising a lipid-PEG-antibody conjugate (e.g., a lipid-PEG-ISVD conjugate, such as a phospholipid-PEG-ISVD conjugate). In some embodiments, the lipid-PEG-ISVD is a phospholipid-PEG-ISVD. In some embodiments, the method comprises the following steps: (a) mixing a first composition comprising monomers of an ISVD comprising a linker (e.g., a linker for click chemistry reaction, such as a cysteine linker (e.g., GGC)), with a second composition comprising a first phospholipid-PEG comprising a bioconjugation linker under conditions that the phospholipid-PEG and the ISVD monomer can form a conjugate through click chemistry; (b) adding a quenching agent to the mixture obtained in step (a) under conditions that the conjugation reaction is quenched, wherein a composition comprising the phospholipid-PEG ISVD conjugate is obtained. As used herein, the term “quenching agent” refers to a compound that is able to compete with at least one of the substrates of the conjugation reaction there to slow down or stop the reaction. For example, the quenching agent can be cysteine when the conjugation click chemistry is based on cysteine-assisted click chemistry. In some embodiments, the composition obtained from the method comprises micelles wherein the micelles comprise the phospholipid-PEG-ISVD.

In some embodiments, the click chemistry reaction takes place in the mixture in step (a) under 15 to 25° C., such as about 15° C., 16° C., 17° C., 18° C., 19° C., 20° C., 21° C., 22° C., 23° C., 24° C., or 25° C. In some embodiments, the click chemistry reaction takes place under 20-22° C.

The click chemistry reaction in step (a) can take as long as needed to ensure a complete reaction. In some embodiments, the reaction takes about 1 to about 3 hours. In some embodiments, the reaction takes about 2 hours.

Step (b) as described in the method is a quenching step in which excessive quenching agent (e.g., cysteine for a cysteine-based click chemistry reaction) is added into the reaction so that the conjugation reaction is slowed down or stopped. In some embodiments, molar ratio between the added quenching agent and the phospholipid-PEG-maleimide is about 5:1 to about 1:1, such as about 5.0:1, 4.9:1, 4.8:1, 4.7:1, 4.6:1, 4.5:1, 4.4:1, 4.3:1, 4.2:1, 4.1:1, 4.0:1, 3.9:1, 3.8:1, 3.7:1, 3.6:1, 3.5:1, 3.4:1, 3.3:1, 3.2:1, 3.1:1, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, or 1.0:1. In some embodiments, the quenching step take about 5 minutes to 60 minutes, such as about 5 minutes, 10 minutes, 15 minutes, 20 minutes, 25 minutes, 30 minutes, 35 minutes, 40 minutes, 45 minutes, 50 minutes, 55 minutes or 60 minutes. In some embodiments, the quenching step takes place in the mixture in step (a) under 15 to 25° C., such as about 15° C., 16° C., 17° C., 18° C., 19° C., 20° C., 21° C., 22° C., 23° C., 24° C., or 25° C. In some embodiments, the click chemistry reaction takes place under 20-22° C.

In some embodiments, at least 50%, at least 55%, at least 60%, at least 65%, at least 70%, at least 75%, at least 80%, at least 85%, at least 90%, at least 95%, at least 99% or more of the ISVDs in the first composition are in monomeric form. In some embodiments, the phospholipid in the first phospholipid-PEG is a derivative of phosphatidylethanolamine. In some embodiments, the phospholipid in the first phospholipid-PEG comprises stearic acid acyl chains. In some embodiments, phospholipid in the first phospholipid-PEG is 1,2-Distearoyl-sn-glycero-3-phosphoethanolamine (DSPE). The PEG in the first phospholipid-PEG can have a weight from about 1 kDa to about 10 kDa, such as about 1 kDa, 1.5 kDa, 2.0 kDa, 2.5 kDa, 3.0 kDa, 3.5 kDa, 4.0 kDa, 4.5 kDa, 5.0 kDa, 5.5 kDa, 6.0 kDa, 6.5 kDa, 7.0 kDa, 7.5 kDa, 8.0 kDa, 8.5 kDa, 9.0 kDa, 9.5 kDa, or 10 kDa. In some embodiments, the PEG in the first phospholipid-PEG has a weight of about 3.4 kDa, and the conjugate is a DSPE-PEG 3.4K-ISVD conjugate. In some embodiments, the ISVD in the DSPE-PEG 3.4K-ISVD conjugate is an anti-CD8α ISVD, such as those described herein.

In some embodiments, the phospholipid-PEG has a bioconjugation linker, so that under proper conditions the first phospholipid-PEG molecules and the ISVD monomers can form a conjugate through click chemistry reaction. In some embodiments, the bioconjugation linker in the phospholipid-PEG has a maleimide group (e.g., phospholipid-PEG-maleimide, such as DSPE-PEG 3.4K-maleimide).

In some embodiments, a mixture obtained from the method described herein that contains micelles comprising the phospholipid-PEG-ISVD is contacted with a composition comprising LNPs. In some embodiments, such contact leads to that molecules in the micelles diffuse from the micelles and insert into the LNPs. For example, the phospholipid-PEG-ISVD in the micelles can diffuse from the micelles and insert into the LNPs to form new LNPs that comprise the phospholipid-PEG-ISVD. Depending on the binding specificity of the ISVD, the resulted LNPs can target to a specific cell type or tissue that the ISVD can specifically bind to. In some embodiments, the LNPs before step (a) comprise elements selected from the group of (i) an ionizable cationic lipid, (ii) a structural lipid, (iii) a neutral phospholipid, (iv) a free PEG-lipid, (v) a payload, such as mRNA, and (vi) any combination thereof. In some embodiments, the resulted LNPs obtained from the method comprise (i) the ionizable cationic lipid, (ii) the structural lipid, (iii) the neutral phospholipid, (iv) the free PEG-lipid, (v) the payload, such as mRNA, and (vi) the phospholipid-PEG-ISVD conjugate. In some embodiments, the LNPs resulted from the method comprise Lipid 15 (ionizable cationic lipid), cholesterol (structural lipid), DSPC (neutral phospholipid), PEG 2000-DPG (same as DPG-PEG 2K; free PEG-lipid), mRNA encodes a chimeric antigen receptor (CAR) comprises an anti-CD22 antibody, such as an anti-CD22 Single-chain variable fragment (ScFv), and DSPE-PEG 3.4K-anti-CD8α ISVD conjugate (lipid-immune cell targeting group conjugate).

Optionally, the composition comprising the phospholipid-PEG that has a bioconjugation linker for the conjugation reaction (e.g., a click chemistry reaction), further contains a second phospholipid-PEG that does not react with the ISVD. For example, the second phospholipid-PEG does not have the bioconjugation linker (e.g., a phospholipid-PEG that is not reactive in the bioconjugation reaction). In some embodiments, the second phospholipid-PEG that does not have the bioconjugation linker stabilizes the composition. For example, in some embodiments, the second phospholipid-PEG that does not have the bioconjugation linker stabilizes the micelle structure of the composition formed by the first phospholipid-PEG having a bioconjugation linker. In some embodiments, the second phospholipid-PEG that does not have the bioconjugation linker is commercially available. In some embodiments, the second phospholipid-PEG that does not have the bioconjugation linker to be used has at least a GMP grade. In some embodiments, the micelles formed through the bioconjugation reaction comprises a conjugate formed by the process as described herein. In some embodiments, the second phospholipid-PEG that does not have the bioconjugation linker stabilizes the micelle structure of the composition formed by the first phospholipid-PEG having a bioconjugation linker and the ISVD, and the second phospholipid-PEG that does not have the bioconjugation linker. In some embodiments, the second phospholipid-PEG that does not have the bioconjugation linker can be the same phospholipid in the first phospholipid-PEG, and the PEG in the second phospholipid-PEG has a different or the same molecular weight as the first phospholipid-PEG that has the bioconjugation linker. In some other embodiments, the second phospholipid-PEG that does not have the bioconjugation linker has a different phospholipid as in the first phospholipid-PEG, and the PEG in the second phospholipid-PEG has a different or the same molecular weight as the first phospholipid-PEG that has the bioconjugation linker. In some embodiments, the PEG in the second phospholipid-PEG has a molecular weight from about 0.5 kDa to about 10 kDa, such as about 0.5 kDa, 1.0 kDa, 1.5 kDa, 2.0 kDa, 2.5 kDa, 3.0 kDa, 3.5 kDa, 4.0 kDa, 4.5 kDa, 5.0 kDa, 5.5 kDa, 6.0 kDa, 6.5 kDa, 7.0 kDa, 7.5 kDa, 8.0 kDa, 8.5 kDa, 9.0 kDa, 9.5 kDa or 10.0 Kda. In some embodiments, the second phospholipid-PEG that does not have the bioconjugation linker can be the same phospholipid in the first phospholipid-PEG, and the PEG in the second phospholipid-PEG has a smaller molecular weight compared to the first phospholipid-PEG that has the bioconjugation linker. In some embodiments, the PEG in the first phospholipid-PEG with a bioconjugation linker has a molecular weight of about 3.4 kDa, and the PEG in the second phospholipid-PEG without a bioconjugation linker has a molecular weight of about 2.0 kDa. In some embodiments, the second phospholipid-PEG is also DSPE-PEG but with a smaller PEG size (e.g., DSPE-PEG2.0k) compared to the first DSPE-PEG having a bioconjugation linker (e.g., DSPE-PEG3.4K-maleimide). In some embodiments, the first DSPE-PEG is DSPE-PEG3.4K-maleimide and the second phospholipid-PEG is DSPE-PEG2.0k-OMeH.

In some embodiments, the first phospholipid-PEG molecules comprising the bioconjugation linker and the second phospholipid-PEG that does not have the bioconjugation linker are mixed first to form micelles before being conjugated to the antibody (e.g., the ISVD). In some embodiments, the molar ratio of the first phospholipid-PEG molecules comprising the bioconjugation linker to the second phospholipid-PEG that does not have the bioconjugation linker before they are mixed to form the micelles, or in the formed micelles is about 1:3 to about 1:1, such as about 1:3, about 2:3, about 1:1. In some embodiments, the molar ratio of the phospholipid-PEG molecules comprising the bioconjugation linker to the second phospholipid-PEG that does not have the bioconjugation linker before they are mixed to form micelles, or the ratio of the two components in formed micelles is about 2:3.

In some embodiments, the formed micelles are conjugated to one or more ISVD monomers that have a linker through a clicking reaction, such as a thiol-maleimide clicking reaction (e.g., through the reaction between a cysteine tag on the ISVD and a maleimide tail on the phospholipid-PEG). In some embodiments, the molar ratio of the ISVD monomers, the phospholipid-PEG molecules comprising the bioconjugation linker, and the second phospholipid-PEG that does not have the bioconjugation linker before the clicking reaction or in the formed conjugate is about 1:1:4 or 1:2:3. In some embodiments, the micelles having the phospholipid-PEG-antibody conjugated to them are mixed with a composition comprising LNPs to form targeted LNPs having the phospholipid-PEG-antibody conjugate.

As a result of the method described herein, micelles comprising the phospholipid-PEG-antibody (e.g., ISVD) conjugate are obtained. In some embodiments, the micelles also comprise a second phospholipid-PEG. In some embodiments, the micelles comprise DSPE-PEG3.4K-antibody (e.g., ISVD) conjugate. In some embodiments, the micelles comprise DSPE-PEG3.4K-ISVD conjugate and DSPE-PEG-3.4-maleimide not conjugated to the antibody (e.g., ISVD). In some embodiments, the micelles comprise DSPE-PEG3.4K-ISVD conjugate, DSPE-PEG-3.4-maleimide not conjugated to the antibody (e.g., ISVD), and/or a second phospholipid-PEG that does not have a bioconjugate linker, such as DSPE-PEG2k. In some embodiments, the DSPE-PEG3.4K-ISVD conjugate has a concentration about 0.084 g/g to 0.15 g/g mRNA in the LNP. In some embodiments, the DSPE-PEG3.4K-ISVD conjugate has a concentration about 0.084 g/g mRNA in the LNP. In some embodiments, the DSPE-PEG3.4K-ISVD conjugate has a concentration about 0.11 g/g mRNA in the LNP. In some embodiments, the DSPE-PEG3.4K-ISVD conjugate has a concentration about 0.05 g/g to 0.15 g/g mRNA in the LNP. In some embodiments, the DSPE-PEG3.4K-ISVD conjugate is DSPE-PEG3.4K-anti-CD8 antibody conjugate.

The obtained micelles comprising the phospholipid-PEG-antibody (e.g., ISVD) can be further used to produce targeted LNPs described in the present disclosure.

In another aspect, also provided is a method for conjugating an antibody (e.g., an ISVD) to a LNP comprising at least one lipid-PEG (e.g., a phospholipid-PEG). In some embodiments, the lipid-PEG-ISVD is a phospholipid-PEG-ISVD. In some embodiments, the method comprises the following steps: (a) mixing a first composition comprising monomers of an ISVD comprising a cysteine containing linker, with a second composition comprising a LNP that contains a first phospholipid-PEG, wherein the first phospholipid-PEG comprises a bioconjugation linker under conditions that the phospholipid-PEG and the ISVD monomer can form a conjugate through click chemistry; (b) adding a quenching agent to the mixture obtained in step (a) under conditions that the conjugation reaction is quenched, wherein a composition comprising LNPs comprising the phospholipid-PEG ISVD conjugate is obtained. In some embodiments, the LNPs that contain first phospholipid-PEG molecules also comprise other elements selected from the group of (i) an ionizable cationic lipid, (ii) a structural lipid, (iii) a neutral phospholipid, (iv) a free PEG-lipid, (v) a payload, such as mRNA, and (vi) any combination thereof. In some embodiments, the LNPs comprise Lipid 15 (ionizable cationic lipid), mRNA (payload), cholesterol (structural lipid), DSPC (neutral phospholipid), and PEG 2000-DPG (same as DPG-PEG 2K; free PEG-lipid). In some embodiments, the mRNA encodes a chimeric antigen receptor (CAR) comprises an anti-CD22 antibody, such as an anti-CD22 Single-chain variable fragment (ScFv). In some embodiments, the LNPs produced from a method as described herein further comprises DSPE-PEG 3.4K-anti-CD8α ISVD conjugate (lipid-immune cell targeting group conjugate).

In some embodiments, the antibody in the phospholipid-PEG-antibody is an immunoglobulin single variable domain. In some embodiments, the antibody in the phospholipid-PEG-antibody is selected from a VHH (including humanized VHH), a VH (including camelized VH, human VH, camelized human VH, dAb) and a VL. In some embodiments, the antibody in the phospholipid-PEG-antibody is an immunoglobulin single variable domain that comprises at least two disulfide bridges.

It is known that all VHHs contain at least one disulfide bridge, between the cysteine residue at position 22 and the cysteine residue at position 92 (numbering according to Kabat; Muyldermans and Lauwereys 1999, J. Mol. Recognit. 12:131). Although most VHHs contain only this single disulfide bridge, it is also known that some VHHs contain a total of two (or in exceptional cases three) disulfide bridges. For example, a class of VHHs referred to as “VHH-1 type”, “VHH-1 class”, or the like (as further defined herein) commonly has a second disulfide bridge between the cysteine residue in CDR2 at position 50 and a cysteine residue present in CDR3 (or in exceptional cases in CDR1 or CDR2). Also, some VHHs derived from camels or dromedaries often have a disulfide bridges between a cysteine residue present in CDR1 (or at position 45 in FR2) and a cysteine residue present in CDR3 (Vu et al. 1997, Mol. Immunol. 34:1121; Muyldermans and Lauwereys 1999). Some VHHs derived from llamas sometimes have a disulfide bridge between cysteine residues present in CDR1 (such as at position 33) and a cysteine residue present in CDR3 (Vu et al., 1997).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one of the framework regions and a cysteine residue in one of the CDR regions.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one of the framework regions and a cysteine residue in CDR3.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in framework two (FR2) and a cysteine residue in CDR3.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 45 in framework two (FR2) and a cysteine residue in CDR3 (as in some VHH's derived from camels and dromedaries).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in one CDR and a cysteine residue in another CDR.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR3 and a cysteine residue in another CDR (in particular in CDR1, as in some VHH's derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in another CDR.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in CDR1.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR1 and a cysteine residue in CDR3 (as in some VHHs derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine at position 33 and a cysteine residue in CDR3 (as in some VHH's derived from camels, dromedaries and llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR2 and a cysteine residue in another CDR.

In some embodiments, the immunoglobulin single variable domain comprises a disulfide bridge between the cysteine residue at position 22 and the cysteine residue at position 92, and further comprises a disulfide bridge that is formed between a cysteine residue in CDR2 and a cysteine residue in CDR2.

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue in CDR2 and a cysteine residue in CDR3 (as in some VHHs derived from llamas).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 50 and a cysteine residue in another CDR, such as CDR1, CDR2 or CDR3 (as in VHHs of the VHH1 type).

In some embodiments, the immunoglobulin single variable domain in the phospholipid-PEG-antibody comprises a disulfide bridge between the cysteine residue at position 22 and the cysteine residue at position 92, and further comprises a disulfide bridge that is formed between a cysteine residue at position 50 and a cysteine residue in CDR3 (as in VHHs of the VHH1 type).

In some embodiments, the immunoglobulin single variable domain in the phospholipid-PEG-antibody may be a “VHH1 type immunoglobulin single variable domain”. An amino acid such as e.g., an immunoglobulin single variable domain or polypeptide is said to be a “VHH1 type immunoglobulin single variable domain” or “VHH type 1 sequence”, if said VHH1 type immunoglobulin single variable domain or VHH type 1 sequence has 85% identity (using the blastp algorithm with standard setting, i.e. blosom62 scoring matrix) to the VHH1 consensus sequence (SEQ ID NO: 569): QVQLVESGGGLVQPGGSLRLSCAASGFTLDYYAIGWFRQAPGKEREGVSCISSSDGSTY YADSVKGRFTISRDNAKNTVYLQMNSLKPEDTAVYYCAA) and mandatorily has a cysteine in position 50, i.e. Cys50 (using Kabat numbering). These VHH1 type immunoglobulin single variable domains generally have (or are capable of forming) a disulfide bridge between Cys50 and a cysteine residue in CDR3 (or in exceptional cases CDR1 or CDR2).

In some embodiments, the immunoglobulin single variable domain in the phospholipid-PEG-antibody is another immunoglobulin single variable domains that comprise two or more disulfide bridges (such as (single) domain antibodies, dAb's, IgNAR domains from sharks, etc.). In some embodiments, the immunoglobulin single variable domain in the phospholipid-PEG-antibody comprises at least two disulfide bridges. In some embodiments, the immunoglobulin single variable domain in the phospholipid-PEG-antibody belongs to the group of VHH1 type immunoglobulin single variable domains.

In some embodiments, the immunoglobulin single variable domain in the phospholipid-PEG-antibody does not belong to the group of VHH1 type immunoglobulin single variable domains, but belongs to another group of immunoglobulin single variable domains, such as the VHH2, VHH3 or any other type immunoglobulin single variable domains.

As such, in some embodiments, the lipid-immune cell targeting group conjugate comprises DSPE-PEG 3.4K-ISVD conjugate. In some embodiments, the lipid-immune cell targeting group conjugate comprises DSPE-PEG 3.4K—VHH conjugate. In some embodiments, the lipid-immune cell targeting group conjugate comprises DSPE-PEG 3.4K-anti-CD8α VHH conjugate. In some embodiments, the lipid-immune cell targeting group conjugate comprises DSPE-PEG 3.4K—VHH1 conjugate. In some embodiments, the lipid-immune cell targeting group conjugate comprises DSPE-PEG 3.4K-anti-CD8α VHH1 conjugate.

In some embodiments, the immunoglobulin single variable domain comprised in the phospholipid-PEG-antibody is an ISVD directed against CD8α, that comprises:
a) the amino acid sequence selected from the group consisting of SEQ ID NOs: 160 to 179;
b) amino acid sequences that have at least 80% amino acid identity with a sequence selected from the group consisting of SEQ ID NOs: 160 to 179, or
c) amino acid sequences that have 3, 2, or 1 amino acid difference with a sequence selected from the group consisting of SEQ ID NOs: 160 to 179;
or any suitable combination thereof.

In some embodiments, the immunoglobulin single variable domain comprised in the phospholipid-PEG-antibody an ISVD against CD8α, which consist of 4 framework regions (FR1 to FR4 respectively) and 3 complementarity determining regions (CDR1 to CDR3 respectively) in which:
(I) CDR1 comprises or essentially consists of an amino acid sequence selected from the group consisting of SEQ ID NOs: 181 and 188, according to Abm definition, and SEQ ID NOs: 251, according to Kabat definition;
or of amino acid sequences that have at least 80%, at least 90%, at least 95%, at least 99% or more sequence identity with a sequence selected from the group consisting of SEQ ID NO: 181 and 188, according to Abm definition, and SEQ ID NOs: 251, according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 181 and 188, according to Abm definition, and SEQ ID NOs: 251, according to Kabat definition;
and/or amino acids sequences that have 2 or only 1 amino acid difference(s) with a sequence selected from the group consisting of SEQ ID NO: 181 and 188, according to Abm definition, and SEQ ID NO: 251, according to Kabat definition, in which any amino acid substitution is a conservative amino acid substitution; and/or said amino acid sequence only contains amino acid substitutions, and no amino acid deletions or insertions, compared to a sequence selected from the group consisting of SEQ ID NO: 181 and 188, according to Abm definition, and SEQ ID NO: 251, according to Kabat definition.
(II) CDR2 comprises or essentially consists of an amino acid sequence comprising a sequence selected from the group consisting of SEQ ID NO: 183 and 190, according to Abm definition, and SEQ ID NOs: 253 and 260, according to Kabat definition,
or amino acid sequences that have at least 80%, at least 90%, at least 95%, at least 99% or more sequence identity with a sequence selected from the group consisting of SEQ ID NO: 183 and 190, according to Abm definition, and SEQ ID NOs: 253 and 260, according to Kabat definition in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 183 and 190, according to Abm definition, and SEQ ID NOs: 253 and 260, according to Kabat definition;
and/or amino acids sequences that have 2 or only 1 amino acid difference(s) with a sequence selected from the group consisting of SEQ ID NO: 183 and 190, according to Abm definition, and SEQ ID NOs: 253 and 260, according to Kabat definition, in which any amino acid substitution is a conservative amino acid substitution; and/or said amino acid sequence only contains amino acid substitutions, and no amino acid deletions or insertions, compared to a sequence selected from the group consisting of SEQ ID NO: 183 and 190, according to Abm definition and SEQ ID NOs: 253 and 260, according to Kabat definition.
(III) CDR3 comprises or essentially consists of an amino acid sequence comprising a sequence selected from the group consisting of SEQ ID NO: 185, 192, 199 and 206, according to Abm definition, and SEQ ID NOs 185, 192, 199 and 206, according to Kabat definition,
or amino acid sequences that have at least 80%, at least 90%, at least 95%, at least 99% or more sequence identity with a sequence selected from the group consisting of SEQ ID NO: 185, 192, 199 and 206, according to Abm definition and SEQ ID NOs: 185, 192, 199 and 206, according to Kabat definition, in which (1) any amino acid substitution is a conservative amino acid substitution; and/or (2) said amino acid sequence only contains amino acids substitutions, and no amino acid deletions or insertions, compared to a sequence selected from the group consisting of SEQ ID NO: 185, 192, 199 and 206, according to Abm definition and SEQ ID NOs: 185, 192, 199 and 206, according to Kabat definition;
and/or amino acids sequences that have 2 or only 1 amino acid difference(s) with a sequence selected from the group consisting of SEQ ID NO: 185, 192, 199 and 206, according to Abm definition and SEQ ID NOs: 185, 192, 199 and 206, according to Kabat definition, in which any amino acid substitution is a conservative amino acid substitution; and/or said amino acid sequence only contains amino acid substitutions, and no amino acid deletions or insertions, compared to a sequence selected from the group consisting of SEQ ID NO: 185, 192, 199 and 206, according to Abm definition and SEQ ID NOs: 185, 192, 199 and 206, according to Kabat definition.

In one embodiment, the immunoglobulin single variable domain comprised in the phospholipid-PEG-antibody comprises a CDR1 that is the amino acid sequence of SEQ ID NO: 244, a CDR2 that is the amino acid sequence of SEQ ID NO: 246 and a CDR3 that is the amino acid sequence of SEQ ID NO: 248 (according to Abm definition) or comprises a CDR1 that is the amino acid sequence of SEQ ID NO: 314, a CDR2 that is the amino acid sequence of SEQ ID NO: 316 and a CDR3 that is the amino acid sequence of SEQ ID NO: 318 (according to Kabat definition).

CD8 binding ISVDs in the phospholipid-PEG-antibody may comprise one, two or all three of the CDRs explicitly listed above. In some embodiments, the anti-CD8α ISVD in the phospholipid-PEG-antibody is selected from the ISVDs described in Table C-1, Table C-2, and Table C-3. In some embodiments, the anti-CD8α ISVD in the phospholipid-PEG-antibody is selected from the ISVDs that have at least 80%, at least 85%, at least 90%, at least 95%, at least 96%, at least 97%, at least 98%, or at least 99% sequence identity with the ISVDs described in Table C-1, Table C-2, and Table C-3. In some embodiments, the anti-CD8α ISVD in the phospholipid-PEG-antibody is selected from the ISVDs that have at least 80%, at least 85%, at least 90%, at least 95%, at least 96%, at least 97%, at least 98%, or at least 99% sequence identity with the ISVDs described in Table C-4.

In some embodiments, the anti-CD8α ISVD comprises or is an ISVD described herein, such as a) the amino acid sequence selected from the group consisting of SEQ ID NOs: 160 to 179; b) amino acid sequences that have at least 80% amino acid identity with a sequence selected from the group consisting of SEQ ID NOs: 160 to 179, or c) amino acid sequences that have 3, 2, or 1 amino acid difference with a sequence selected from the group consisting of SEQ ID NOs: 160 to 179. In some embodiments, the anti-CD8α ISVD comprises SEQ ID NO: 9, SEQ ID NO: 169 or SEQ ID NO: 179.

In some embodiments, the phospholipid in the phospholipid-PEG-antibody is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), ceramide. In some embodiments, the phospholipid is DSPE.

In some embodiments, the antibody is covalently coupled to the phospholipid in Formula (II) ([Lipid]-[optional linker]-[antibody]), phospholipid-linker-antibody, or phospholipid-PEG-antibody, wherein the linker comprises polyethylene glycol (PEG). In some embodiments, PEG has a molecular weight of about 1.0 kDa to about 6 kDa. In some embodiments, the PEG has a molecular weight about 3400 Da (e.g., PEG 3400 (PEG 3.4K)). In some embodiments, phospholipid-PEG molecule is conjugated to the antibody through a click chemistry reaction. In some embodiments, the antibody is an ISVD. The phospholipid-PEG can be conjugated to the ISVD according to methods described herein.

The lipid-PEG-antibody conjugate of the present disclosure can be inserted into an LNP to form a targeted LNP. In some embodiment, the lipid-PEG-antibody conjugate can help the LNP to target a specific cell, a specific tissue, or a specific organ. In some embodiments, the lipid-PEG-antibody conjugate comprises an immune cell targeting group. For example, the antibody in the conjugate can be an antibody that specifically binds to immune cells. In some embodiments, the lipid-immune cell targeting group conjugate in the LNP of the present disclosure comprises DSPE-PEG-anti-CD8α antibody. In some embodiments, the conjugate comprises DSPE-PEG3.4K-anti-CD8α antibody. In some embodiments, the conjugate comprises DSPE-PEG3.4K-A044300805_v8 (SEQ ID NO: 9, SEQ ID NO: 169 or SEQ ID NO: 179).

In some embodiments, the conjugate of the present disclosure (e.g., DSPE-PEG3.4K-A044300805_v8) can be present in the LNP in a range of 0.001-0.5 mol percent, 0.001-0.1 mol %, 0.01-0.5 mol %, 0.05-0.5 mol %, 0.1-0.5 mol %, 0.1-0.3 mol %, 0.1-0.2 mol %, 0.2-0.3 mol %, of about 0.01 mol %, about 0.05 mol %, about 0.1 mol %, about 0.15 mol %, about 0.2 mol %, about 0.25 mol %, about 0.3 mol %, about 0.35 mol %, about 0.4 mol %, about 0.45 mol %, or about 0.5 mol %. In some embodiments, the conjugate comprises DSPE-PEG3.4K-A044300805_v8, and is present in the LNP in a range of 0.01 to 0.02 mol percent. In some embodiment, the conjugate comprising DSPE-PEG3.4K-A044300805_v8 is present in the LNP is about 0.015 to about 0.016 mol percent.

### B. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA) disposed therein to cells, e.g., mammalian cells, e.g., immune cells. In some embodiments, an ionizable cationic lipid of the present disclosure is used to produce LNPs as described herein. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the substituents of the ionizable amine head group are chosen to tune the apparent pKa of amine head group in the LNP formulation to fall in the range of 6-8, such as between 6.2-7.4, or between 6.7-7.2. As such, the amine head groups remains strongly cationic under acidic formulation conditions (e.g., pH 4-pH 5.5), neutral or slightly anionic or slightly cationic (typical zeta potential of 0±5 mV) in physiological pH (7.4), but strongly cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, the present invention provides a compound represented by Formula (I):

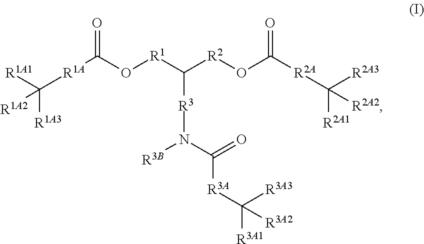

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

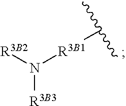

R^{3B1} is C₁₋₆ alkylene; and
R^{3B2} and R^{3B3} are each independently H, unsubstituted C₁₋₆ alkyl, or C₁₋₆ alkyl substituted with 1 or 2 —OH.

In some embodiments, provided is a compound of Formula (I-1):

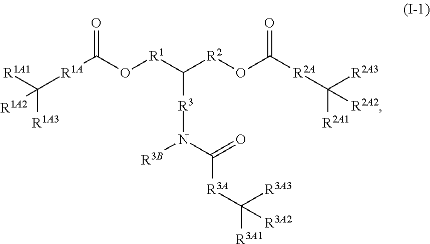

or a salt thereof, wherein:
R¹, R², and R³ are each independently a bond or methylene;
R^{1A} and R^{2A} are each C₁₋₁₀ alkylene;
R^{3A} is C₁₋₅ alkylene;
R^{1A1}, R^{1A2}, R^{2A1}, R^{2A2}, R^{3A1}, and R^{3A2} are each H;
R^{1A3} and R^{2A3} are each C₁₋₂₀ alkenyl;
R^{3A3} is-C(O)O(C₁₋₂₀ alkyl);
R^{3B} is

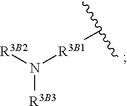

R^{3B1} is C₂₋₄ alkylene; and
R^{3B2} and R^{3B3} are each methyl.

In some embodiments, R^{3B1} is —(CH₂)₃—. In some embodiments, R^{3A} is methylene or ethylene. In some embodiments, R^{3A} is methylene. In some embodiments, R^{3A3} is —C(O)O(C₅₋₁₅ alkyl). In some embodiments, R^{3A3} is —C(O)O(C₁₀ alkyl).

In some embodiments, the ionizable cationic lipid is

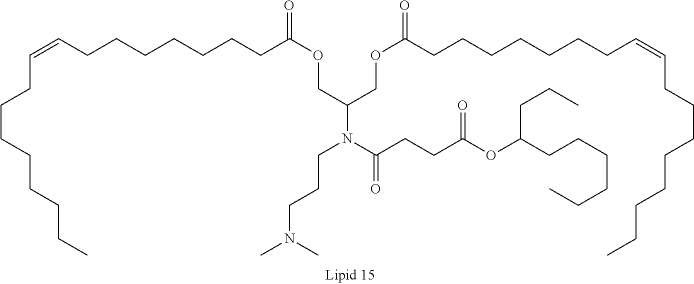 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In one aspect, the present invention provides a compound of Formula (I-A):

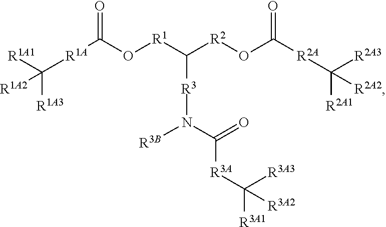

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

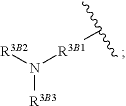

R^{3B1} is C₁₋₆ alkylene; and
R^{3B2} and R^{3B3} are each independently H, unsubstituted C₁₋₆ alkyl, or C₁₋₆ alkyl substituted with one or more substituents each independently selected from the group consisting of —OH and —O—(C₁₋₆ alkyl).

Any of the variables or substituents provided herein is unsubstituted or substituted with one or more substituents. In some embodiments, any of the variables or substituents provided herein is optionally substituted. In some embodiments, any of the variables or substituents provided herein is optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

In some embodiments, R¹, R², and R³ are each independently a bond or C₁₋₃ alkylene. In some embodiments, R¹, R², and R³ are each independently a bond or methylene. In some embodiments, R¹ and R² are each methylene and R³ is a bond. In some embodiments, R¹, R², and R³ are each methylene. In some embodiments, R¹, R², and R³ are each independently unsubstituted or substituted. In some embodiments, R¹, R², and R³ are unsubstituted.

In some embodiments, R^{1A}, R^{2A}, and R^{3A} are each independently a bond or C₁₋₁₀ alkylene. In some embodiments, R¹⁴, R^{2A}, and R^{3A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each a bond, each —CH₂—, each —(CH₂)₂—, each —(CH₂)₃—, each —(CH₂)₄—, each —(CH₂)₅—, each —(CH₂)₆—, each (CH₂)₇—, or each —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{1A} and R^{2A} are each a bond, each —(CH₂)₂—, each —(CH₂)₄—, each —(CH₂)₆—, each —(CH₂)₇—, or each —(CH₂)₈—. In some embodiments, R^{3A} is a bond, —CH₂—, —(CH₂)₂—, or —(CH₂)₇—. In some embodiments, R^{1A}, R^{2A}, and R^{3A} are each independently unsubstituted or substituted. In some embodiments, R^{1A}, R^{2A}, and R³A are unsubstituted.

In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₁₅ alkyl, —CH≡CH—(C₁₋₁₅ alkyl), —CH═CH—CH₂—CH═CH—(C₁₋₁₀ alkyl), —(CH₂)₀₋₄C(O)OCH(C₁₋₁₀ alkyl) (C₁₋₁₅ alkyl), —(CH₂)₀₋₄OC(O)CH (C₁₋₁₀ alkyl) (C₁₋₁₅ alkyl), —(CH₂)₀₋₄C(O)OCH₂ (C₁₋₁₅ alkyl), or —(CH₂)₀₋₄OC(O)CH₂ (C₁₋₁₅ alkyl). In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each independently unsubstituted or substituted. In some embodiments, R^{1A1}, R^{1A2}, R^{1A3}, R^{2A1}, R^{2A2}, R^{2A3}, R^{3A1}, R^{3A2}, R^{3A3}, R¹, R², R³, R^{1A}, R^{2A}, and R^{3A} are each unsubstituted. In some embodiments, R^{1A1}, R^{1A2}, R^{1A3}, R^{2A1}, R^{2A2}, R^{2A3}, R^{3A1}, R^{3A2}, and R^{3A3} are each independently unsubstituted or substituted. In some embodiments, R^{1A1}, R^{1A2}, R^{1A3}, R^{2A1}, R^{2A2}, R^{2A3}, R^{3A1}, R^{3A2}, and R^{3A3} are each unsubstituted. In some embodiments, R¹, R², R³, R^{1A}, R^{2A}, and R^{3A} are each independently unsubstituted or substituted. In some embodiments, R¹, R², R³, R^{1A}, R^{2A}, and R^{3A} are each unsubstituted. In some embodiments, R¹, R², and R³ are each unsubstituted.

In some embodiments, R^{3B1} is unsubstituted. In some embodiments, R^{3B1} is not substituted with oxo.

In some embodiments, R^{1A1} and R^{2A1} are each independently-CH═CH—(C₁₋₁₅ alkyl), —CH—CH—CH₂—CH═CH—(C₁₋₁₀ alkyl), —(CH₂)₀₋₄C(O)OCH(C₁₋₁₀ alkyl) (C₁₋₁₅ alkyl), or —(CH₂)₀₋₄OC(O)CH (C₁₋₁₀ alkyl) (C₁₋₁₅ alkyl); and R^{1A2}, R^{1A3}, R^{2A2}, and R^{2A3} are each H. In some embodiments, R^{1A1} and R^{2A1} are each —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl) (C₁₋₁₅ alkyl), or —(CH₂)₀₋₄OC(O)CH (C₁₋₁₀ alkyl) (C₁₋₁₅ alkyl); and R^{1A2}, R^{1A3}, R^{2A2}, and R^{2A3} are each H. In some embodiments, R^{1A1} and R^{2A1} are each

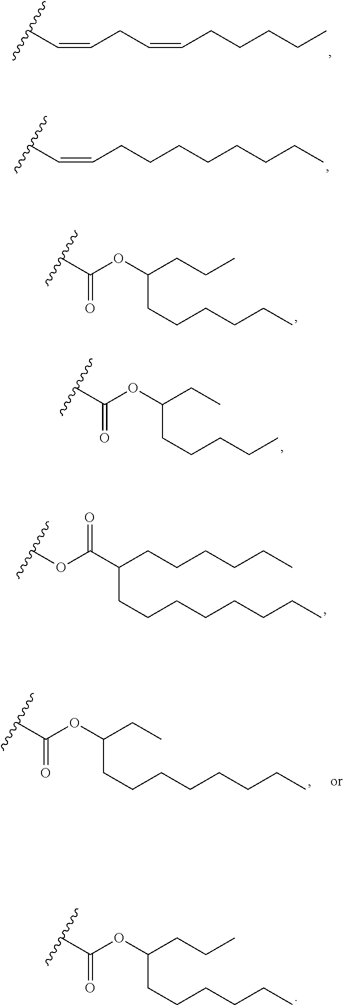

In some embodiments, R^{1A1} and R^{2A1} are each

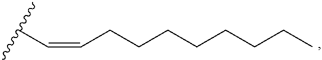 [SMILES: */C=C\CCCCCCCC]

In some embodiments, R^{1A2}, R^{1A3}, R^{2A2}, and R^{2A3} are each H.

In some embodiments, R^{1A1} and R^{2A1} are each C₁₋₁₅ alkyl; R^{1A2} and R^{2A2} are each C₁₋₁₅ alkyl; and R^{1A3} and R^{2A3} are each H. In some embodiments, R^{1A1} and R^{2A1} are each

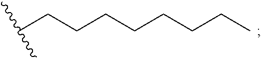 [SMILES: *CCCCCCCC]

and R^{1A2} and R^{2A2} are each

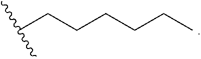 [SMILES: *CCCCCC]

In some embodiments, R^{1A3} and R^{2A3} are each H. In some embodiments, R^{1A} and R^{2A} are each a bond.

In some embodiments, R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂ (C₁₋₁₅ alkyl); and R^{1A3} and R^{2A3} are each H. In some embodiments, R^{1A1} and R^{2A1} are each

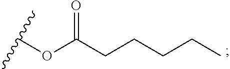 [SMILES: *OC(=O)CCCCC]

and R^{2A1} and R^{2A2} are each

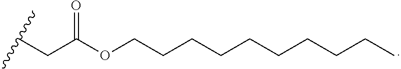 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{1A1} and R^{2A1} are each —C(O)OCH₂(C₁₋₁₅ alkyl); R^{1A2} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

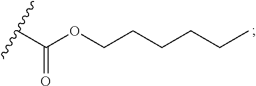 [SMILES: *C(=O)OCCCCCC]

and R^{1A2} and R^{2A2} are each

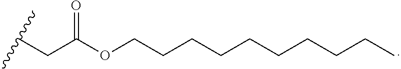 [SMILES: *CC(=O)OCCCCCCCCCC]

In some embodiments, R^{1A1} and R^{2A1} are each

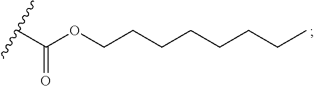 [SMILES: *C(=O)OCCCCCCCC]

and R^{2A1} and R^{2A2} are each

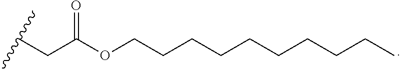 [SMILES: *CC(=O)OCCCCCCCCCC]

In some embodiments, R^{1A3} and R^{2A3} are each H. In some embodiments, R^{1A} and R^{2A} are each a bond.

In some embodiments, R^{3A1}, R^{3A2}, and R^{3A3} are each independently H, C₁₋₁₅ alkyl, —(CH₂)₀₋₄C(O)OCH(C₁₋₅ alkyl) (C₁₋₁₀ alkyl), —(CH₂)₀₋₄OC(O)CH (C₁₋₅ alkyl) (C₁₋₁₀ alkyl), —(CH₂)₀₋₄C(O)OCH₂ (C₁₋₁₀ alkyl), or —(CH₂)₀₋₄OC(O)CH₂ (C₁₋₁₀ alkyl).

In some embodiments, R^{3A1} and R^{3A2} are each independently C₁₋₁₅ alkyl; and R^{3A3} is H. In some embodiments, R^{3A1} and R^{3A2} are each independently ethyl, propyl, butyl, pentyl, hexyl, or heptyl. In some embodiments, R^{3A1} and R^{3A2} are each independently ethyl,

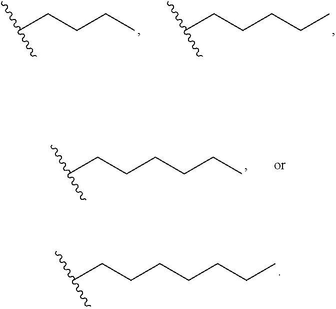

In some embodiments, R^{3A3} is H. In some embodiments, R^{3A} is a bond.

In some embodiments, R^{3A1} is C₁₋₁₅ alkyl; and R^{3A2} and R^{3A3} are each H. In some embodiments, R^{3A1} is

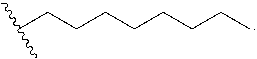 [SMILES: *CCCCCCCC]

In some embodiments, R^{3A2} and R^{3A3} are each H. In some embodiments, R^{3A} is a bond.

In some embodiments, R^{3A1} is —C(O)OCH(C₁₋₅ alkyl)(C₁₋₁₀ alkyl); and R^{3A2} and R^{3A3} are each H. In some embodiments, R^{3A1} is

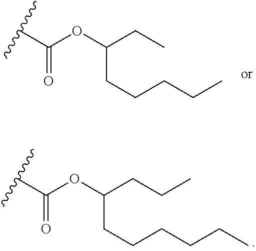 [SMILES: *C(=O)OC(CC)CCCCC ; *C(=O)OC(CCC)CCCCCC]

In some embodiments, R^{3A1} is

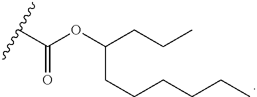 [SMILES: *C(=O)OC(CCC)CCCCCC]

In some embodiments, R^{3A} is ethylene or —(CH₂)₂—. In some embodiments, R^{3A2} and R^{3A3} are each H.

In some embodiments, R^{3A1} is —(CH₂)₀₋₄OC(O)CH₂ (C₁₋₁₀ alkyl); R^{3A2} is —(CH₂)₀₋₄(O)OCH₂(C₁₋₁₀ alkyl); and R^{3A3} is H. In some embodiments, R^{3A1} is

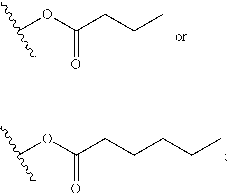

### and R^{3A2} is

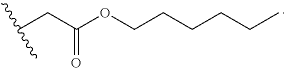 [SMILES: *CC(=O)OCCCCCC]

In some embodiments, R^{3A3} is H. In some embodiments, R^{3A} is a bond.

In some embodiments, R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H. In some embodiments, R^{3A1} is

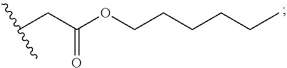 [SMILES: *CC(=O)OCCCCCC]

### and R^{3A2} is

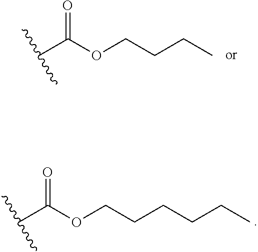

In some embodiments, R^{3A3} is H. In some embodiments, R^{3A} is a bond.

In some embodiments, R^{3A1}, R^{3A2}, and R^{3A3} are each H.

R^{a1} and R^{a2} are each independently C₁₋₂₀ alkyl or C₁₋₂₀ alkenyl. In some embodiments, R^{a1} and R^{a2} are each independently-(CH₂)₀₋₁₅CH₃ or —CH(C₁₋₁₀ alkyl) (C₁₋₁₅ alkyl). In some embodiments, R^{a1} and R^{a2} are each independently

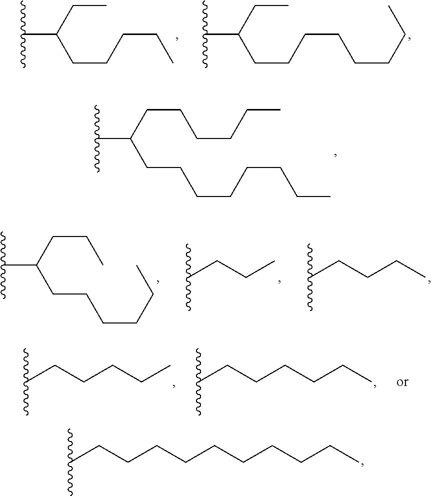

each of which is optionally substituted. In some embodiments, R^{a1} and R^{a2} are each independently unsubstituted or substituted. In some embodiments, R^{a1} and R^{a2} are unsubstituted.

In some embodiments, R^{3B} is

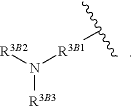

In some embodiments, R^{3B} is H. In some embodiments, R^{3B} is unsubstituted or substituted. In some embodiments, R^{3B} is unsubstituted.

In some embodiments, R^{3B1} is C₁₋₆ alkylene. In some embodiments, R^{3B1} is ethylene or propylene. In some embodiments, R^{3B1} is unsubstituted or substituted. In some embodiments, R^{3B1} is optionally substituted.

In some embodiments, R^{3B2} and R^{3B3} are each independently and optionally substituted. In some embodiments, R^{3B2} and R^{3B3} are each independently H or C₁₋₆ alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O) NR^{s10}R¹¹, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more-OH. In some embodiments, R^{3B2} and R^{3B3} are each methyl or each ethyl, each optionally substituted with one or more-OH. In some embodiments, R^{3B2} and R^{3B3} are each unsubstituted methyl.

In some embodiments,

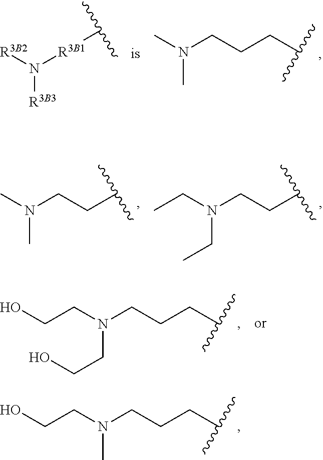

each of which is optionally substituted.

In one aspect, the present invention provides a compound represented by Formula (Ia):

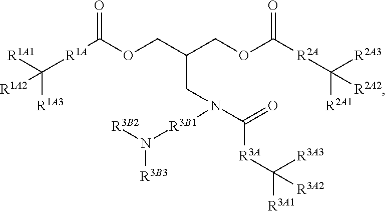

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{3B1}, R^{3B2}, and R^{3B3} are as defined for Formula (I) or any variation or embodiment thereof.

In one aspect, the present invention provides a compound represented by Formula (Ib):

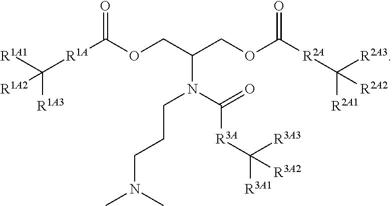

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, and R^{3A3} are as defined for Formula (I) or any variation or embodiment thereof.

In some embodiments, the cationic lipid in a targeted LNP of the present disclosure has a concentration between about 10 mol % to about 60 mol % of the LNP, such as about 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, 55 mol %, 56 mol %, 57 mol %, 58 mol %, 59 mol %, or 60 mol %. In some embodiments, the cationic lipid in a targeted LNP of the present disclosure has a concentration between about 48.0 mol % to about 50.0 mol %, such as 48.0 mol %, 48.1 mol %, 48.2 mol %, 48.3 mol %, 48.4 mol %, 48.5 mol %, 48.6 mol %, 48.7 mol %, 48.8 mol %, 48.9 mol %, 49.0 mol %, 49.1 mol %, 49.2 mol %, 49.3 mol %, 49.4 mol %, 49.5 mol %, 49.6 mol %, 49.7 mol %, 49.8 mol %, 49.9 mol %, or 50.0 mol %.

In some embodiments, the cationic lipid in the targeted LNP comprises Lipid 15.

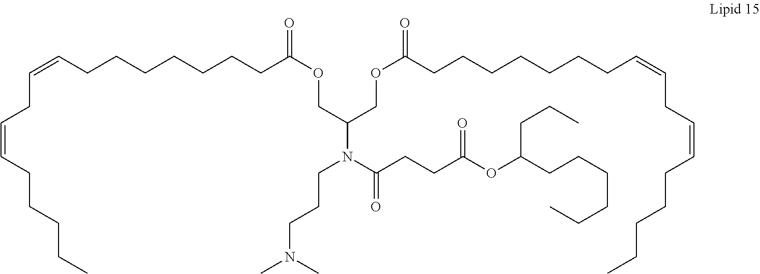 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

In some aspects, the ionizable lipid is DLin-KC2-DMA:

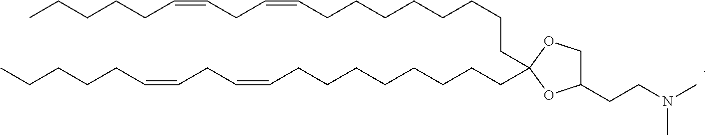 [SMILES: CCCCC/C=C\C/C=C\CCCCCCCCC1(CCCCCCCC/C=C\C/C=C\CCCCC)OCC(CCN(C)C)O1]

In some embodiments, the ionizable lipid is DLin-KC3-DMA:

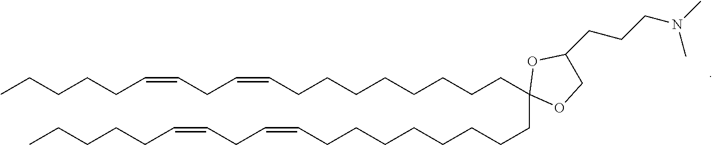 [SMILES: CCCCC/C=C\C/C=C\CCCCCCCCC1(CCCCCCCC/C=C\C/C=C\CCCCC)OCC(CCCN(C)C)O1]

In some embodiments, the ionizable lipid is DLin-MC3-DMA:

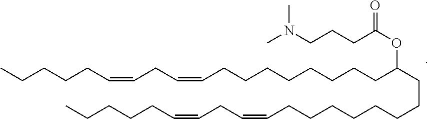 [SMILES: CCCCC/C=C\C/C=C\CCCCCCCCC(CCCCCCCC/C=C\C/C=C\CCCCC)OC(=O)CCCN(C)C]

In some embodiments, the cationic lipid, or a salt thereof, is a compound selected from the compounds listed in Table 1, or a slat thereof.

TABLE 1
(Lipid 1)

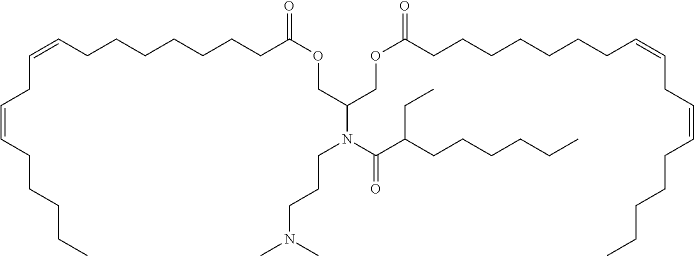 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

Lipid 1
(Lipid 2)

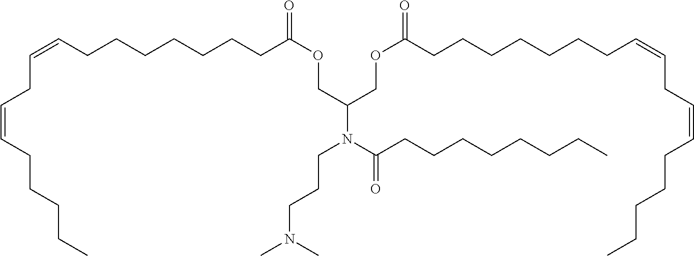 [SMILES: CCCCC/C=C\C/C=C\CCCCCCCC(=O)OCC(COC(=O)CCCCCCC/C=C\C/C=C\CCCCC)N(CCCN(C)C)C(=O)CCCCCCCC]

Lipid 2
(Lipid 3)

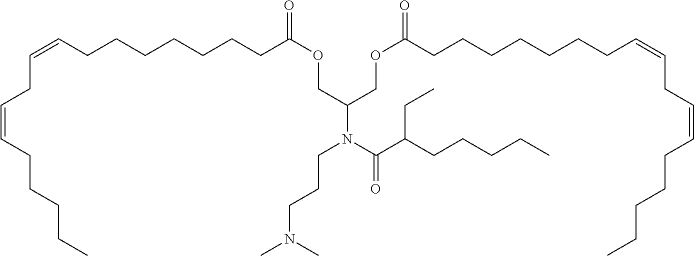 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

Lipid 3
(Lipid 4)

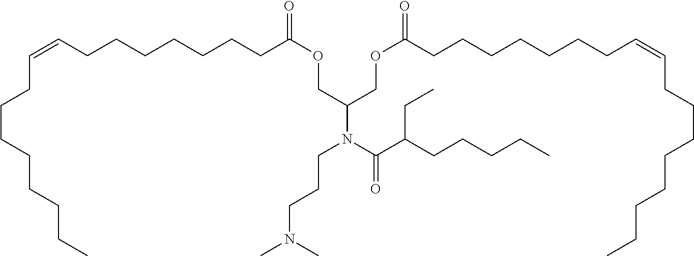 [SMILES: CCCCCCCC/C=C\CCCCCCCC(=O)OCC(COC(=O)CCCCCCC/C=C\CCCCCCCC)N(CCCN(C)C)C(=O)C(CC)CCCCC]

Lipid 4
(Lipid 5)

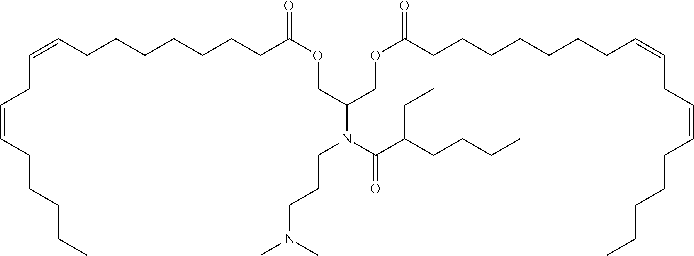 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCC]

Lipid 5
(Lipid 5A)

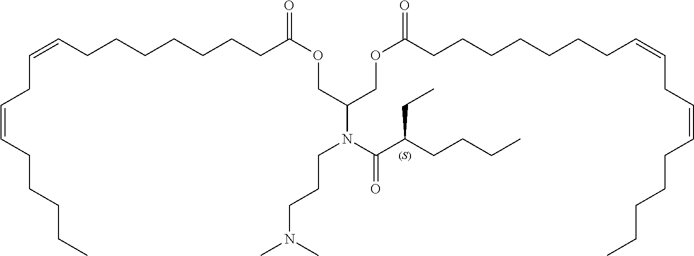

Lipid 5A
(Lipid 6)

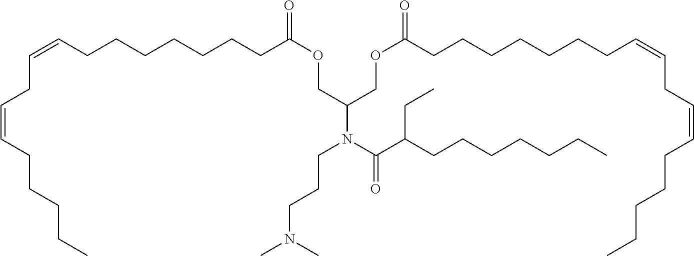 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

Lipid 6
(Lipid 7)

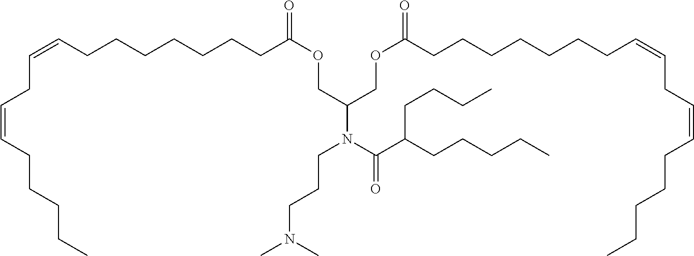 [SMILES: CCCCC/C=C\C/C=C\CCCCCCCC(=O)OCC(COC(=O)CCCCCCC/C=C\C/C=C\CCCCC)N(CCCN(C)C)C(=O)C(CCCC)CCCCC]

Lipid 7
(Lipid 8)

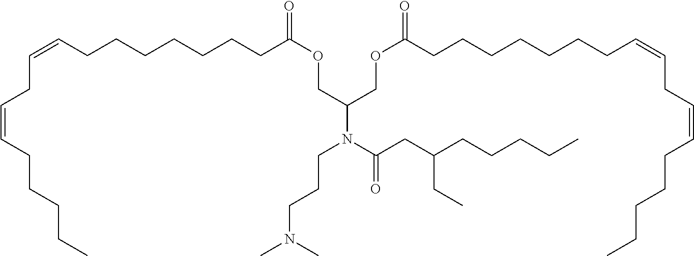 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

Lipid 8
(Lipid 9)

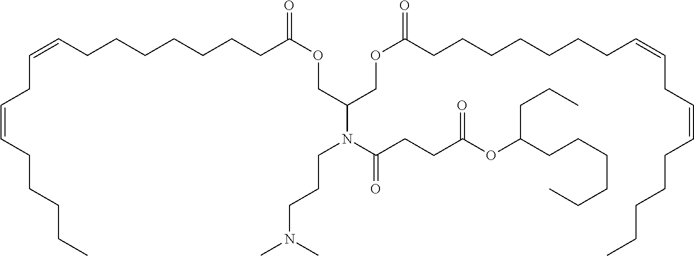 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

Lipid 9
(Lipid 10)

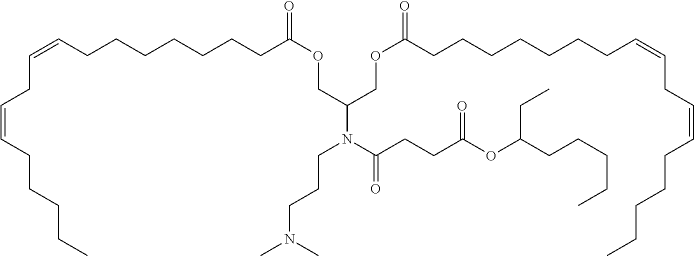 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CC)CCCCC]

Lipid 10
(Lipid 10A)

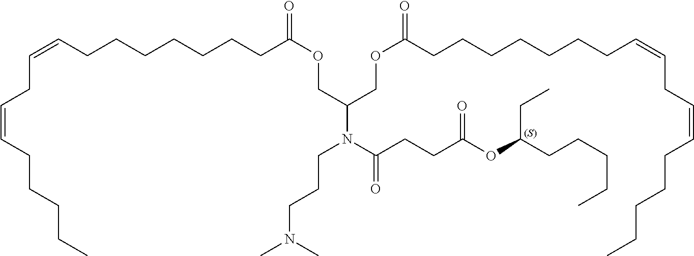

Lipid 10A
(Lipid 11)

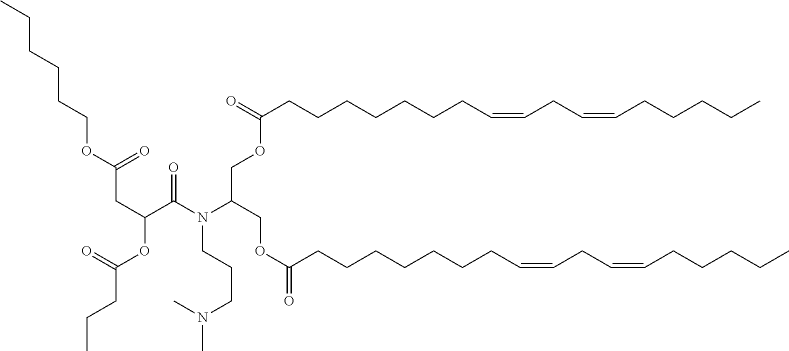 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCC]

Lipid 11
(Lipid 11A)

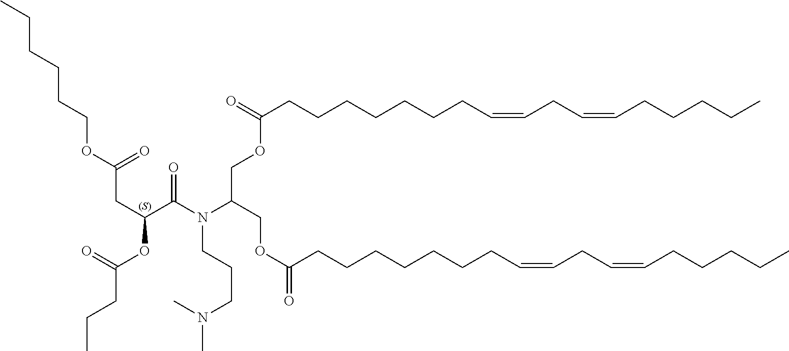

Lipid 11A
(Lipid 12)

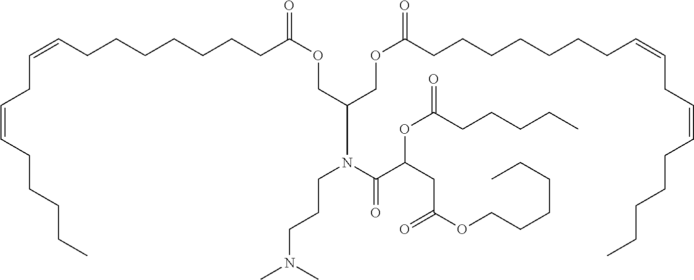 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 12
(Lipid 13)

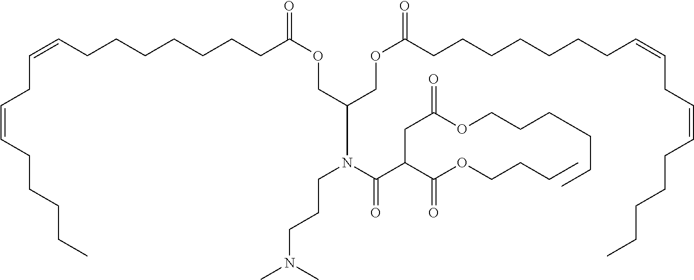 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCC]

Lipid 13
(Lipid 14)

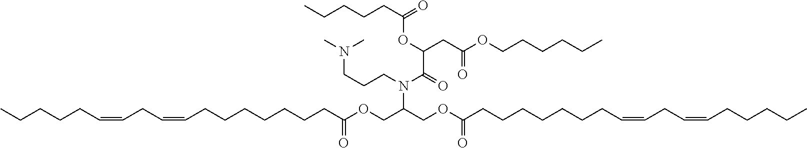 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 14
(Lipid 14A)

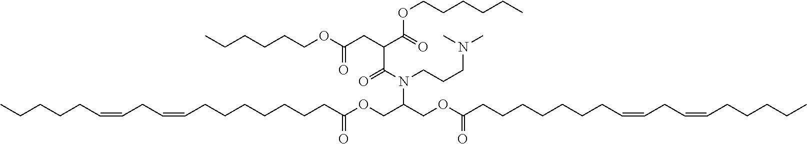 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCCCC]

Lipid 14A
(Lipid 15)

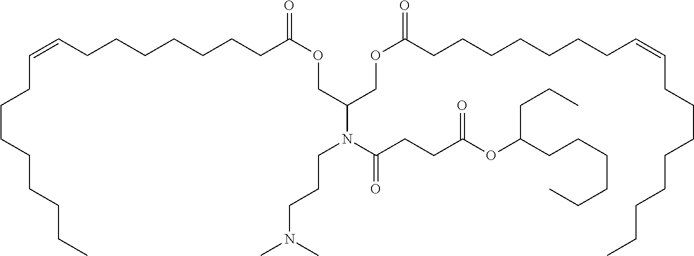 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

Lipid 15
(Lipid 16)

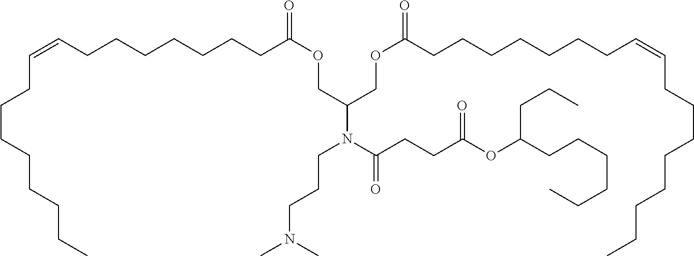 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

Lipid 16
(Lipid 17)

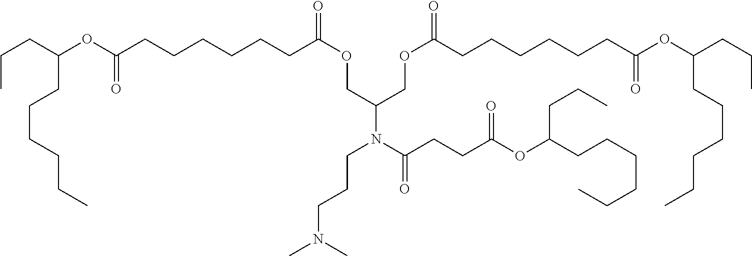 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 17
(Lipid 17A)

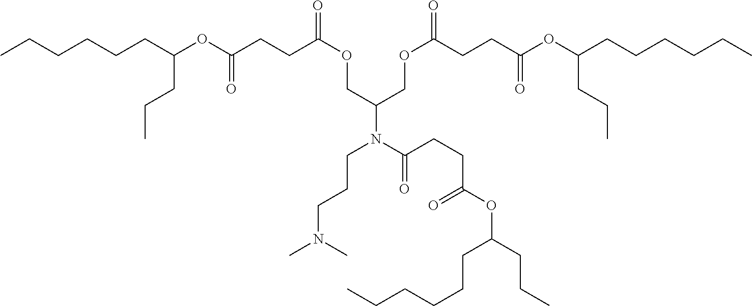 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

Lipid 17A
(Lipid 18)

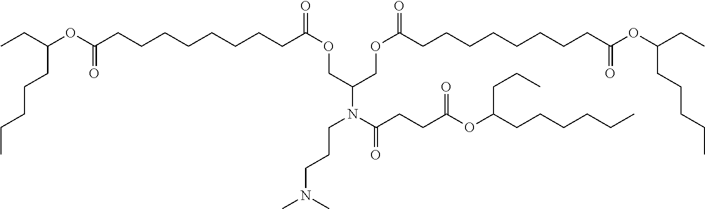 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

Lipid 18
(Lipid 18A)

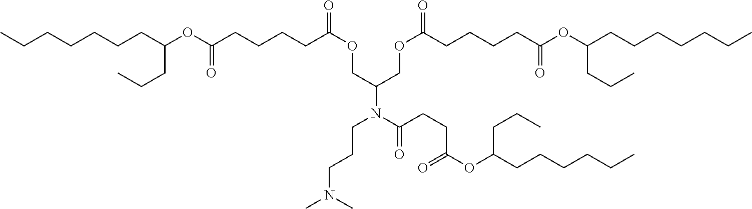 [SMILES: CCCCCCCC(CCC)OC(=O)CCCCC(=O)OCC(COC(=O)CCCCC(=O)OC(CCC)CCCCCCC)N(CCCN(C)C)C(=O)CCC(=O)OC(CCC)CCCCCC]

Lipid 18A
(Lipid 19)

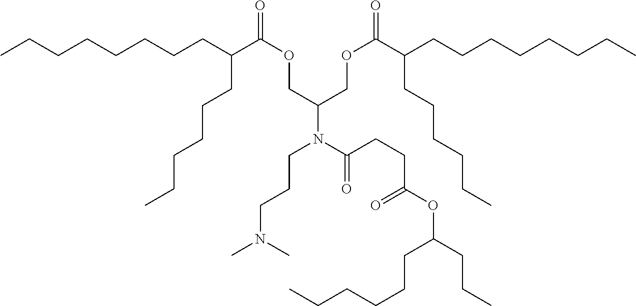 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC]

Lipid 19
(Lipid 19A)

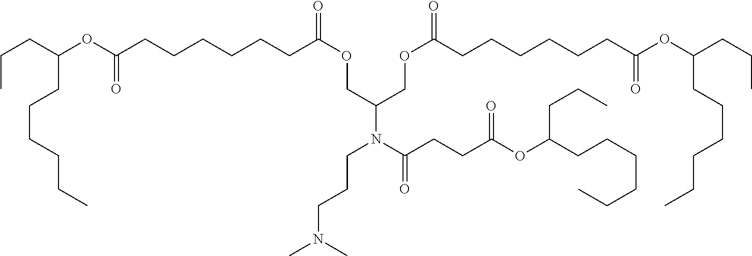 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 19A
(Lipid 20)

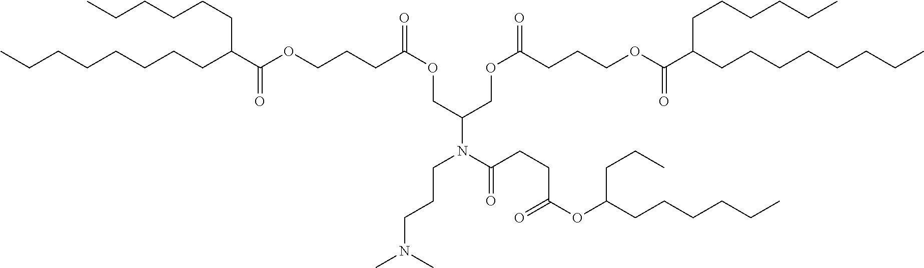 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

Lipid 20
(Lipid 20A)

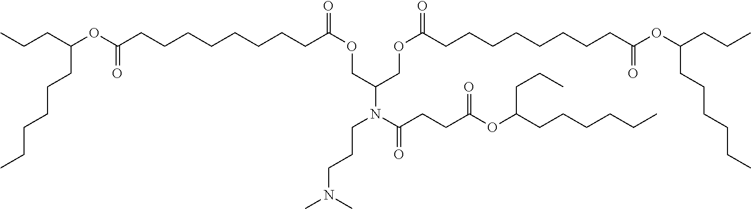 [SMILES: CCCCCCC(CCC)OC(=O)CCCCCCCCC(=O)OCC(COC(=O)CCCCCCCCC(=O)OC(CCC)CCCCCC)N(CCCN(C)C)C(=O)CCC(=O)OC(CCC)CCCCCC]

Lipid 20A
(Lipid 21)

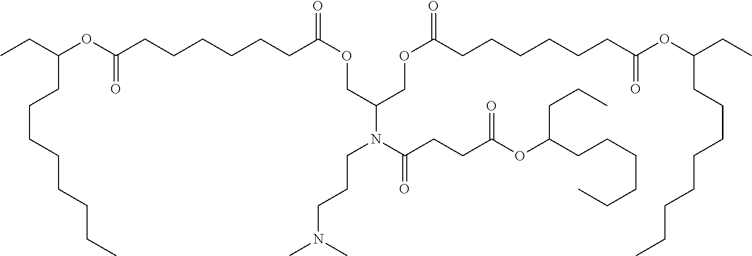 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

Lipid 21
(Lipid 21A)

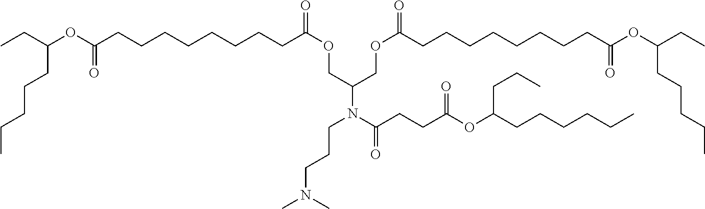 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

Lipid 21A
(Lipid 22)

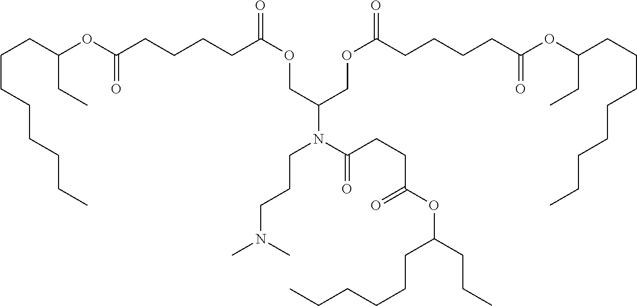 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

Lipid 22
(Lipid 23)

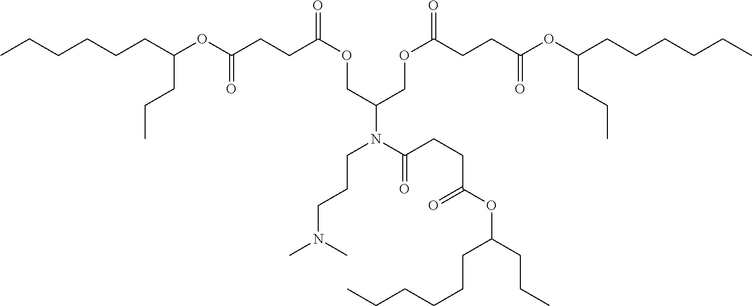 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

Lipid 23
(Lipid 23A)

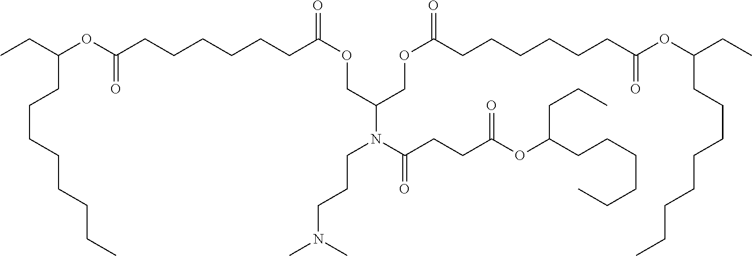 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

Lipid 23A
(Lipid 24)

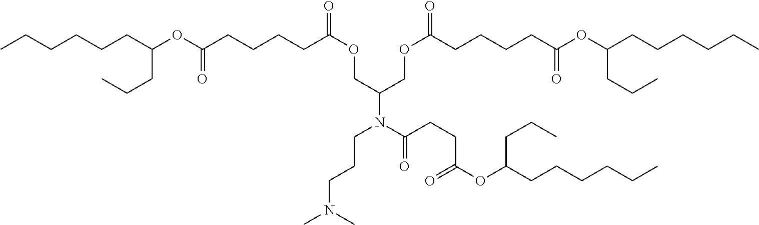 [SMILES: CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

Lipid 24
(Lipid 24A)

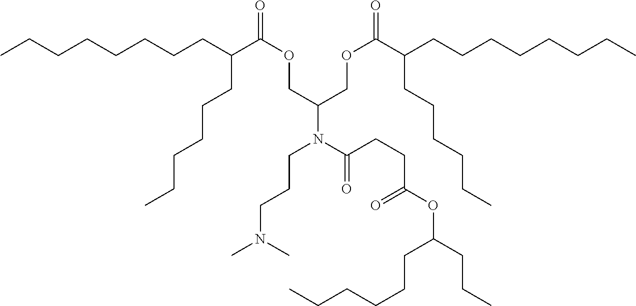 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC]

Lipid 24A
(Lipid 25)

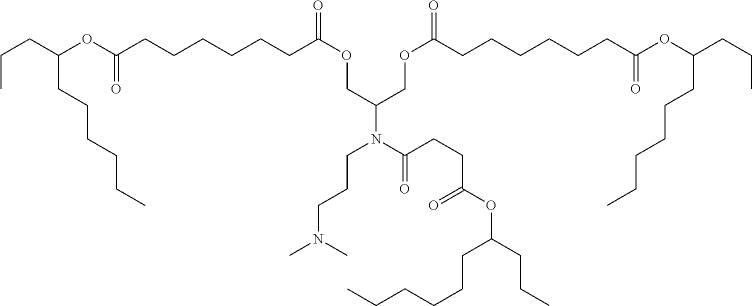 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 25
(Lipid 25A)

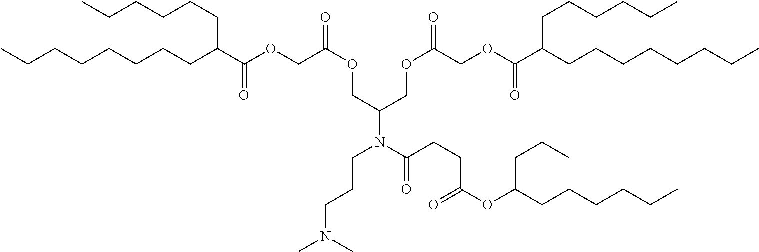 [SMILES: CCCCCCCCC(CCCCCC)C(=O)OCC(=O)OCC(COC(=O)COC(=O)C(CCCCCC)CCCCCCCC)N(CCCN(C)C)C(=O)CCC(=O)OC(CCC)CCCCCC]

Lipid 25A
(Lipid 26)

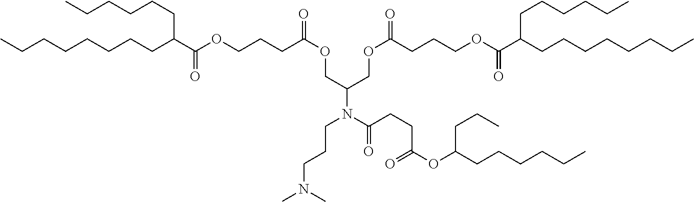 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

Lipid 26
(Lipid 27)

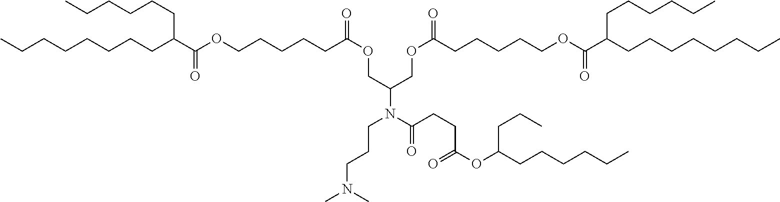 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CCC)CCCCCC]

Lipid 27
(Lipid 28)

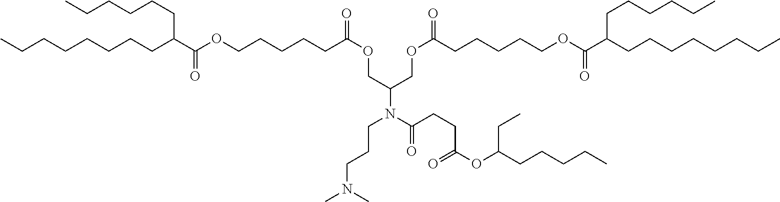 [SMILES: CCCCCCCCC(CCCCCC)C(=O)OCCCCCC(=O)OCC(COC(=O)CCCCCOC(=O)C(CCCCCC)CCCCCCCC)N(CCCN(C)C)C(=O)CCC(=O)OC(CC)CCCCC]

Lipid 28
(Lipid 29)

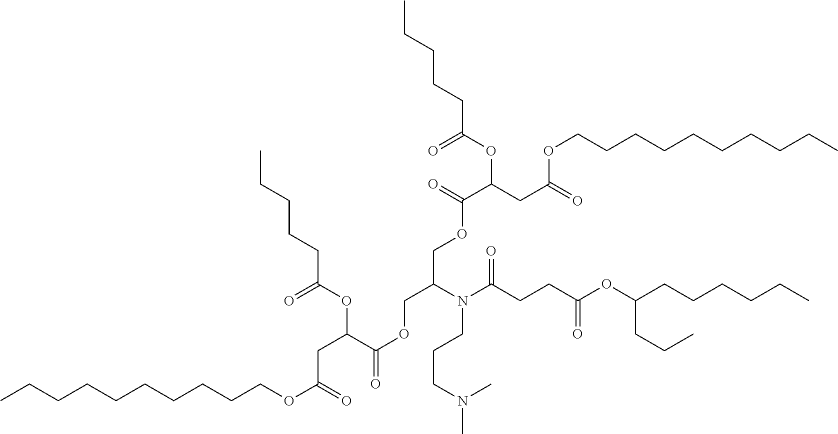 [SMILES: CCCCCCCCCCOC(=O)CC(OC(=O)CCCCC)C(=O)OCC(COC(=O)C(CC(=O)OCCCCCCCCCC)OC(=O)CCCCC)N(CCCN(C)C)C(=O)CCC(=O)OC(CCC)CCCCCC]

Lipid 29
(Lipid 30)

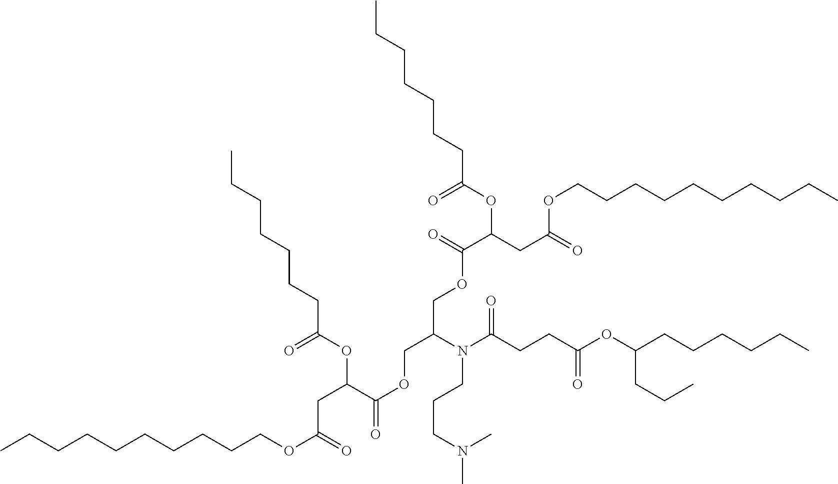 [SMILES: CCCCCCCCCCOC(=O)CC(OC(=O)CCCCCCC)C(=O)OCC(COC(=O)C(CC(=O)OCCCCCCCCCC)OC(=O)CCCCCCC)N(CCCN(C)C)C(=O)CCC(=O)OC(CCC)CCCCCC]

Lipid 30
(Lipid 31)

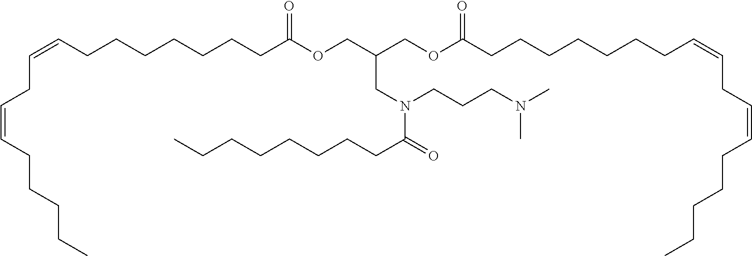 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC]

Lipid 31
(Lipid 32)

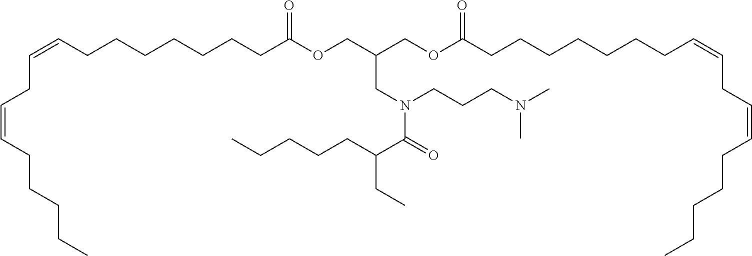 [SMILES: CCCCC/C=C\C/C=C\CCCCCCCC(=O)OCC(COC(=O)CCCCCCC/C=C\C/C=C\CCCCC)CN(CCCN(C)C)C(=O)C(CC)CCCCC]

Lipid 32
(Lipid 33)

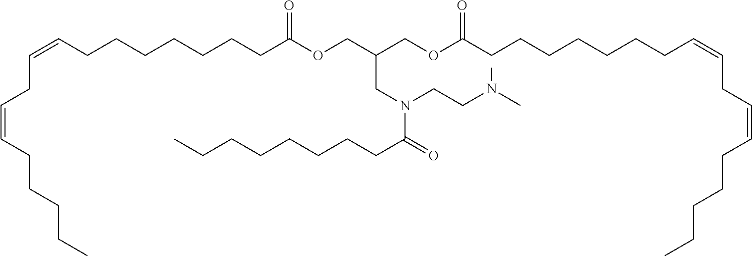 [SMILES: CCCCC/C=C\C/C=C\CCCCCCCC(=O)OCC(COC(=O)CCCCCCC/C=C\C/C=C\CCCCC)CN(CCN(C)C)C(=O)CCCCCCCC]

Lipid 33
(Lipid 34)

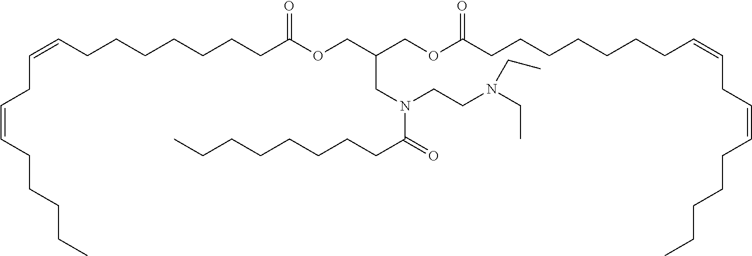 [SMILES: CCCCC/C=C\C/C=C\CCCCCCCC(=O)OCC(COC(=O)CCCCCCC/C=C\C/C=C\CCCCC)CN(CCN(CC)CC)C(=O)CCCCCCCC]

Lipid 34
(Lipid 35)

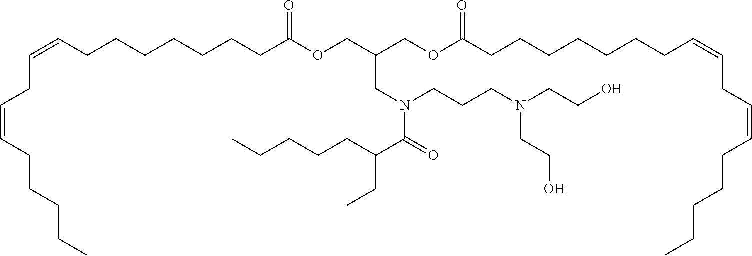 [SMILES: CCCCC/C=C\C/C=C\CCCCCCCC(=O)OCC(COC(=O)CCCCCCC/C=C\C/C=C\CCCCC)CN(CCCN(CCO)CCO)C(=O)C(CC)CCCCC]

Lipid 35
(Lipid 36)

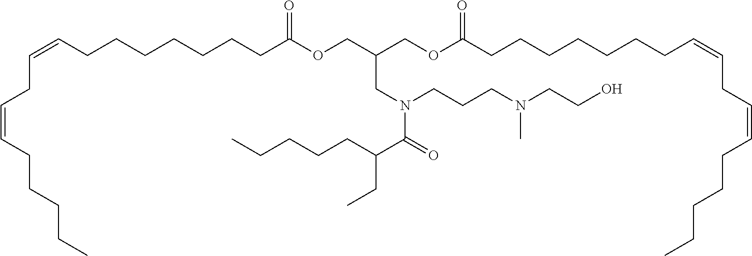 [SMILES: CCCCC/C=C\C/C=C\CCCCCCCC(=O)OCC(COC(=O)CCCCCCC/C=C\C/C=C\CCCCC)CN(CCCN(C)CCO)C(=O)C(CC)CCCCC]

Lipid 36
(Lipid 37)

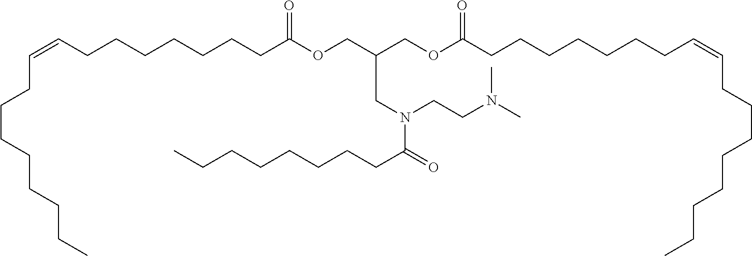 [SMILES: CCCCCCCC/C=C\CCCCCCCC(=O)OCC(COC(=O)CCCCCCC/C=C\CCCCCCCC)CN(CCN(C)C)C(=O)CCCCCCCC]

Lipid 37
(Lipid 38)

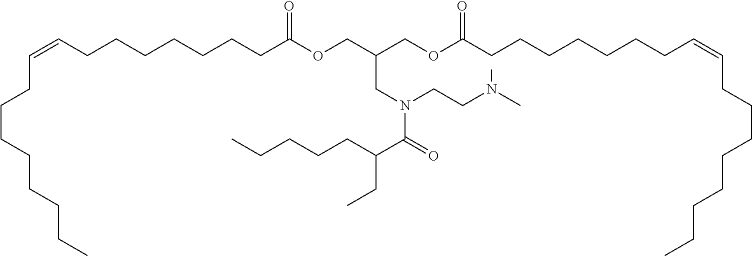 [SMILES: CCCCCCCC/C=C\CCCCCCCC(=O)OCC(COC(=O)CCCCCCC/C=C\CCCCCCCC)CN(CCN(C)C)C(=O)C(CC)CCCCC]

Lipid 38
(Lipid 37A)

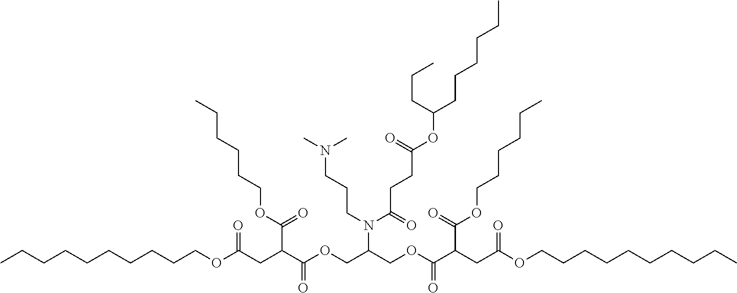 [SMILES: CCCCCCCCCCOC(=O)CC(C(=O)OCCCCCC)C(=O)OCC(COC(=O)C(CC(=O)OCCCCCCCCCC)C(=O)OCCCCCC)N(CCCN(C)C)C(=O)CCC(=O)OC(CCC)CCCCCC]

Lipid 37A
(Lipid 38A)

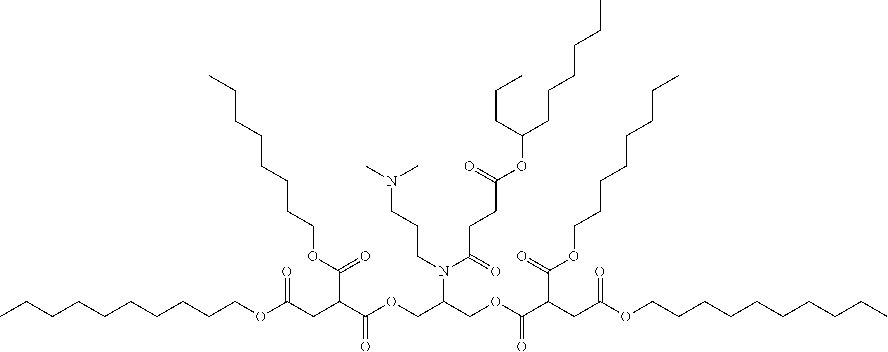 [SMILES: CCCCCCCCCCOC(=O)CC(C(=O)OCCCCCCCC)C(=O)OCC(COC(=O)C(CC(=O)OCCCCCCCCCC)C(=O)OCCCCCCCC)N(CCCN(C)C)C(=O)CCC(=O)OC(CCC)CCCCCC]

Lipid 38A

In some aspects, the ionizable lipid comprises one or more lipids as described in International PCT Publication Nos. WO2009028824A2, WO2011043913A2, WO2012091523A2, WO2015074085A1, WO2016081029A1, WO2017117530A1, WO2018118102A1, WO2018119163A1, WO2019045897A1, WO2020069445A1, WO2020106903A1, WO2020219876A1, WO2021055892A1, WO2021163339A1, WO2021188389A2, WO2021202694A1, WO2022016070A1, WO2022119883A2, WO2022120388A2, WO2022159475A1, WO2022207938A1, WO2022218957A1, WO2022246555A1, WO2023023410A2, WO2023091490A1, WO2023091787A1, WO2023114937A2, WO2023114944A1, WO2023115221A1, WO2023121970A1, WO2023121975A1, WO2023141624A1, WO2023144798A1, WO2023183616A1, WO2023196444A1, WO2023196527A2, WO2023207101A1, WO2023235589A1, WO2023240156A1, WO2024008967A1, WO2024010330A1, WO2024019770A1, WO2025076113A1, and WO2024035710A2, each of which is hereby incorporated by reference in their entirety.

### C. Payload

In some embodiments, the payload is a drug substance. In some embodiments, the payload is a nucleic acid. In some embodiments, the nucleic acid is RNA. In some embodiments, the RNA is mRNA. In some embodiments, mRNA encodes a synthetic T cell receptor (synTCR) or a Chimeric Antigen Receptor (CAR). In some embodiments, mRNA comprises a 5′ Cap, a 5′ untranslated region (UTR), a payload nucleic acid sequence encoding a payload polypeptide, a 3′ UTR, and a polyA tail. In some embodiments, the payload polypeptide comprises an antibody specifically binding to B-cell, a hinge and transmembrane domain, a co-stimulatory domain, and a signaling domain.

In some embodiments, the payload is an mRNA that encodes a payload polypeptide comprising: an optional Lead peptide sequence (e.g., a signal peptide), an antibody heavy chain variable region (VH), an antibody light chain variable region (VL), a hinge domain, a transmembrane domain, a Co-stimulatory domain, and a Signaling domain. In some embodiments, each of the domains described herein is connected to another directly without any linker. In some embodiments, one or more linkers are used to connect any two of the domains described herein. In some embodiments, the payload is an mRNA that encodes a payload polypeptide having the following formula, arranged from N-terminus to C-terminus:

[Lead peptide sequence (optional)]-[antibody heavy chain variable region (VH)]-[Linker A (optional)]-[antibody light chain variable region (VL)]-[Linker B (optional)]-[Hinge domain]-[Transmembrane domain]-[Co-stimulatory domain]-[Signaling domain]. In some embodiments, exemplary payload polypeptides are demonstrated in FIG. 45.

In some embodiments, the payload is an mRNA that comprises a 5′ Cap (optional), a 5′ UTR (optional), nucleotides encoding a Lead polypeptide sequence, nucleotides encoding VH, nucleotides encoding a Linker A, nucleotides encoding VL, nucleotides encoding a Linker B, nucleotides encoding a Hinge, nucleotides encoding a Transmembrane domain, nucleotides encoding Co-stimulatory domain, nucleotides encoding a Signaling domain, a 3′ UTR (optional), and polyA tail (optional). In some embodiments, the payload has the following formula, arranged from 5′ to 3′:

5′ Cap (optional)-5′ UTR (optional)-nucleotides encoding Lead peptide sequence (optional)-nucleotides encoding VH-nucleotides encoding a Linker A (optional)-nucleotides encoding VL-nucleotides encoding Linker B (optional)-nucleotides encoding a Hinge-nucleotides encoding a Transmembrane domain-nucleotides encoding Co-stimulatory domain-nucleotides encoding Signaling domain-3′ UTR (optional)-polyA tail (optional).

In some embodiments, the 5′ UTR comprises SEQ ID NO: 516 or SEQ ID NO: 518. In some embodiments, the 3′ UTR comprises SEQ ID NO: 517 or SEQ ID NO: 519. In some embodiments, the 5′ UTR comprises SEQ ID NO: 516 and the 3′ UTR comprises SEQ ID NO: 517. In some embodiments, the 3′ UTR comprises SEQ ID NO: 518 and the 3′ UTR comprises SEQ ID NO: 519.

In some embodiments, an mRNA of the present disclosure comprises a 5′ cap structure. In some embodiments, the 5′ cap structure is IRES, Cap0, Capl, ARCA, inosine, Nl-methyl-guanosine, 2′fluoro-guanosine, 7-deaza-guanosine, CleanCap™, m7(3′OMeG)(5′)ppp(5′)(2′OMeA)pG, 8-oxo-guanosine, 2-amino-guanosine, LNA-guanosine, 2-azido-guanosine, Cap2, Cap4, CAP-003, or CAP-225. In one embodiment, the mRNA comprises a 5′-cap and wherein at least one of the uridines in the molecule is a modified uridine, preferably Nl-methyl-pseudouridine (1m Ψ). In some embodiments, the mRNA comprises a 5′ cap having the sequence NpppNU, wherein the U in the 5′ cap is an unmodified uridine. In one embodiment, the 5′ cap has the sequence NpppAU with A representing a modified or unmodified adenosine nucleotide. For example, a modified nucleotide N or A 3′ to the triphosphate linkage may have a modified ribose structure such as a 2′-O-methylated ribose (Nm or Am) resulting in a so-called “Cap 1”. In contrast, a cap comprising a nucleotide N or A 3′ to the triphosphate linkage having an unmethylated ribose is usually referred to as “Cap 0”.

In some embodiments, the antibody specifically binding to B-cell is an antibody that specifically binds to human CD22. In some embodiments, the antibody specifically binding to B-cell comprises an antibody heavy chain variable (VH) domain and an antibody light chain variable (VL) domain. In some embodiments, the CD22 antibody comprises HC CDR1, HC CDR2 and HC CDR3 of the heavy chain variable region set forth in SEQ ID NO: 433, 481, 495, or 509. In some embodiments, the CD22 antibody further comprises LC CDR1, LC CDR2 and LC CDR3 of the light chain variable region set forth in SEQ ID NO: 434, 482, 496, or 510. In some embodiments, the CD22 antibody comprises HC CDR1, HC CDR2 and HC CDR3 of the heavy chain variable region set forth in SEQ ID NO: 433, 481, 495, or 509, and LC CDR1, LC CDR2 and LC CDR3 of the light chain variable region set forth in SEQ ID NO: 434, 482, 496, or 510.

In some embodiments, the CD22 antibody comprises HC CDR1, HC CDR2, and HC CDR3 according to Kabat definition selected from the group consisting of:
(i) (a) HC CDR1 is or comprises the amino acid sequence depicted in SEQ ID NO: 427, (b) HC CDR2 is or comprises the amino acid sequence depicted in SEQ ID NO: 428, and (c) HC CDR3 is or comprises the amino acid sequence depicted in SEQ ID NO: 429;
(ii) (a) HC CDR1 is or comprises the amino acid sequence depicted in SEQ ID NO: 475, (b) HC CDR2 is or comprises the amino acid sequence depicted in SEQ ID NO: 476, and (c) HC CDR3 is or comprises the amino acid sequence depicted in SEQ ID NO: 477;
(iii) (a) HC CDR1 is or comprises the amino acid sequence depicted in SEQ ID NO: 489, (b) HC CDR2 is or comprises the amino acid sequence depicted in SEQ ID NO: 490, and (c) HC CDR3 is or comprises the amino acid sequence depicted in SEQ ID NO: 491; or
(iv) (a) HC CDR1 is or comprises the amino acid sequence depicted in SEQ ID NO: 503, (b) HC CDR2 is or comprises the amino acid sequence depicted in SEQ ID NO: 504, and (c) HC CDR3 is or comprises the amino acid sequence depicted in SEQ ID NO: 505.

In some embodiments, the CD22 antibody comprises LC CDR1, LC CDR2, and LC CDR3 according to Kabat definition selected from the group consisting of:
(i) (a) LC CDR1 is or comprises the amino acid sequence depicted in SEQ ID NO: 430, (b) LC CDR2 is or comprises the amino acid sequence depicted in SEQ ID NO: 431, and (c) LC CDR3 is or comprises the amino acid sequence depicted in SEQ ID NO: 432;
(ii) (a) LC CDR1 is or comprises the amino acid sequence depicted in SEQ ID NO: 478, (b) LC CDR2 is or comprises the amino acid sequence depicted in SEQ ID NO: 479, and (c) LC CDR3 is or comprises the amino acid sequence depicted in SEQ ID NO: 480;
(iii) (a) LC CDR1 is or comprises the amino acid sequence depicted in SEQ ID NO: 492, (b) LC CDR2 is or comprises the amino acid sequence depicted in SEQ ID NO: 493, and (c) LC CDR3 is or comprises the amino acid sequence depicted in SEQ ID NO: 494; or
(iv) (a) LC CDR1 is or comprises the amino acid sequence depicted in SEQ ID NO: 506, (b) LC CDR2 is or comprises the amino acid sequence depicted in SEQ ID NO: 507, and (c) LC CDR3 is or comprises the amino acid sequence depicted in SEQ ID NO: 508.

In some embodiments, the CD22 antibody according to Kabat definition comprises:
(i) (a) HC CDR1 is or comprises the amino acid sequence depicted in SEQ ID NO: 427, (b) HC CDR2 is or comprises the amino acid sequence depicted in SEQ ID NO: 428, (c) HC CDR3 is or comprises the amino acid sequence depicted in SEQ ID NO: 429 (d) LC CDR1 is or comprises the amino acid sequence depicted in SEQ ID NO: 430, (e) LC CDR2 is or comprises the amino acid sequence depicted in SEQ ID NO: 431, and (f) LC CDR3 is or comprises the amino acid sequence depicted in SEQ ID NO: 432;
(ii) (a) HC CDR1 is or comprises the amino acid sequence depicted in SEQ ID NO: 475, (b) HC CDR2 is or comprises the amino acid sequence depicted in SEQ ID NO: 476, (c) HC CDR3 is or comprises the amino acid sequence depicted in SEQ ID NO: 477, (d) LC CDR1 is or comprises the amino acid sequence depicted in SEQ ID NO: 478, (e) LC CDR2 is or comprises the amino acid sequence depicted in SEQ ID NO: 479, and (f) LC CDR3 is or comprises the amino acid sequence depicted in SEQ ID NO: 480;
(iii) (a) HC CDR1 is or comprises the amino acid sequence depicted in SEQ ID NO: 489, (b) HC CDR2 is or comprises the amino acid sequence depicted in SEQ ID NO: 490, (c) HC CDR3 is or comprises the amino acid sequence depicted in SEQ ID NO: 491, (d) LC CDR1 is or comprises the amino acid sequence depicted in SEQ ID NO: 492, (e) LC CDR2 is or comprises the amino acid sequence depicted in SEQ ID NO: 493, and (f) LC CDR3 is or comprises the amino acid sequence depicted in SEQ ID NO: 494; or
(iv) (a) HC CDR1 is or comprises the amino acid sequence depicted in SEQ ID NO: 503, (b) HC CDR2 is or comprises the amino acid sequence depicted in SEQ ID NO: 504, (c) HC CDR3 is or comprises the amino acid sequence depicted in SEQ ID NO: 505, (d) LC CDR1 is or comprises the amino acid sequence depicted in SEQ ID NO: 506, (e) LC CDR2 is or comprises the amino acid sequence depicted in SEQ ID NO: 507, and (f) LC CDR3 is or comprises the amino acid sequence depicted in SEQ ID NO: 508.

In some embodiments, the CD22 antibody comprises:
(i) the heavy chain variable region sequence set forth in SEQ ID NO: 433 and the light chain variable region sequence set forth in SEQ ID NO: 434 or SEQ ID NO: 514;
(ii) the heavy chain variable region sequence set forth in SEQ ID NO: 481 and the light chain variable region sequence set forth in SEQ ID NO: 482;
(iii) the heavy chain variable region sequence set forth in SEQ ID NO: 495 and the light chain variable region sequence set forth in SEQ ID NO: 496;
(iv) the heavy chain variable region sequence set forth in SEQ ID NO: 509 and the light chain variable region sequence set forth in SEQ ID NO: 510;
(v) the heavy chain variable region sequence set forth in SEQ ID NO: 447 and the light chain variable region sequence set forth in SEQ ID NO: 434 or SEQ ID NO: 448;
(vi) the heavy chain variable region sequence set forth in SEQ ID NO: 451 and the light chain variable region sequence set forth in SEQ ID NO: 434 or SEQ ID NO: 452;
(vii) the heavy chain variable region sequence set forth in SEQ ID NO: 457 and the light chain variable region sequence set forth in SEQ ID NO: 434 or SEQ ID NO: 458.

In some embodiments, the CD22 antibody comprises SEQ ID NO: 433 and SEQ ID NO: 514.

In some embodiments, the CD22 antibody is selected from the antibodies below:

TABLE D
Additional CD22 antibodies

| CD22 Antibody | | Sequences | SEQ ID NO |
|---|---|---|---|
| CD22- | HC CDR1 | SYGMH | 427 |
| mAb-4^{∧} | HC CDR2 | IIYYDGSKKYYADSVKG | 428 |
| | HC CDR3 | ELTGDAFDI | 429 |
| | LC CDR1 | RASQSIGSSLH | 430 |
| | LC CDR2 | YASQSFS | 431 |
| | LC CDR3 | HQSSTLPYT | 432 |
| | VH | QVQLVESGGGVVQPGRSLRLSCAASGFTFSSYGMHWV | 433 |
| | | RQAPGKGLEWVAIIYYDGSKKYYADSVKGRFTISRDNS | |
| | | KNTLY | |
| | | LQMNSLRAEDTAVYYCARELTGDAFDIWGQGTKVTVS | |
| | | S | |
| | VL | EIVLTQSPDFQSVTPKEKVTITCRASQSIGSSLHWYQQK | 434 |
| | | PDQSPKLLIKYASQSFSGVPSKFIGSGSGTDFTLTINSLEA | |
| | | EDAATFYCHQSSTLPYTFGQGTKLEIK | |
| | HC Constant | ASTKGPSVFPLAPSSKSTSGGTAALGCLVKDYFPEPVTV | 435 |
| | Region | S | |
| | | WNSGALTSGVHTFPAVLQSSGLYSLSSVVTVPSSSLGTQ | |
| | | TYICNVNHKPSNTKVDKRVEPKSCDKTHTCPPCPAPELL | |
| | | GG | |
| | | PSVFLFPPKPKDTLMISRTPEVTCVVVDVSHEDPEVKFN | |
| | | W | |
| | | YVDGVEVHNAKTKPREEQYNSTYRVVSVLTVLHQDWL | |
| | | NGK | |
| | | EYKCKVSNKALPAPIEKTISKAKGQPREPQVYTLPPSREE | |
| | | MTKNQVSLTCLVKGFYPSDIAVEWESNGQPENNYKTTP | |
| | | PV | |
| | | LDSDGSFFLYSKLTVDKSRWQQGNVFSCSVMHEALHN | |
| | | HYT | |
| | | QKSLSLSPG | |
| | LC Constant | RTVAAPSVFIFPPSDEQLKSGTASVVCLLNNFYPREAKV | 436 |
| | Region | Q | |
| | | WKVDNALQSGNSQESVTEQDSKDSTYSLSSTLTLSKAD | |
| | | YEKHKVYACEVTHQGLSSPVTKSFNRGEC | |
| | Heavy Chain | QVQLVESGGGVVQPGRSLRLSCAASGFTFSSYGMHWV | 437 |
| | | RQAPGKGLEWVAIIYYDGSKKYYADSVKGRFTISRDNS | |
| | | KNTLY | |
| | | LQMNSLRAEDTAVYYCARELTGDAFDIWGQGTKVTVS | |
| | | SAS | |
| | | TKGPSVFPLAPSSKSTSGGTAALGCLVKDYFPEPVTVSW | |
| | | N | |
| | | SGALTSGVHTFPAVLQSSGLYSLSSVVTVPSSSLGTQTYI | |
| | | CNVNHKPSNTKVDKRVEPKSCDKTHTCPPCPAPELLGG | |
| | | PS | |
| | | VFLFPPKPKDTLMISRTPEVTCVVVDVSHEDPEVKFNW | |
| | | YV | |
| | | DGVEVHNAKTKPREEQYNSTYRVVSVLTVLHQDWLNG | |
| | | KEY | |
| | | KCKVSNKALPAPIEKTISKAKGQPREPQVYTLPPSREEM | |
| | | T | |
| | | KNQVSLTCLVKGFYPSDIAVEWESNGQPENNYKTTPPV | |
| | | LD | |
| | | SDGSFFLYSKLTVDKSRWQQGNVFSCSVMHEALHNHY | |
| | | TQK | |
| | | SLSLSPG | |
| | Light Chain | EIVLTQSPDFQSVTPKEKVTITCRASQSIGSSLHWYQQKP | 438 |
| | | DQSPKLLIKYASQSFSGVPSKFIGSGSGTDFTLTINSLEA | |
| | | EDAATFYCHQSSTLPYTFGQGTKLEIKRTVAAPSVFIFPP | |
| | | SDEQLKSGTASVVCLLNNFYPREAKVQWKVDNALQSG | |
| | | NSQ | |
| | | ESVTEQDSKDSTYSLSSTLTLSKADYEKHKVYACEVTH | |
| | | QGLSSPVTKSFNRGEC | |
| CD22- | HC CDR1 | GYYIH | 475 |
| mAb-1^{∧} | HC CDR2 | WINPNSGGTNYAQKFQG | 476 |
| | HC CDR3 | EDTWNYIDY | 477 |
| | LC CDR1 | RASQSISSHLN | 478 |
| | LC CDR2 | AASSLHS | 479 |
| | LC CDR3 | QQSYSTPRT | 480 |
| | VH | QVQLVQSGAEVKKPGASVKVSCKASGYTFTGYYIHWV | 481 |
| | | RQAPGQGLEWMGWINPNSGGTNYAQKFQGRVTMTR | |
| | | DTSIGTAYMELSRLRSDDTAIYYCTREDTWNYIDYWGQ | |
| | | GTLVTVSS | |
| | VL | DIQMTQSPSSLSASVGDRVTITCRASQSISSHLNWYQQK | 482 |
| | | PGKAPKLLIVAASSLHSGVPSRFSGSGSGTDFTLTISSLQ | |
| | | PEDFATYCCQQSYSTPRTFGQGTKVEIK | |
| | HC Constant | ASTKGPSVFPLAPSSKSTSGGTAALGCLVKDYFPEPVTV | 435 |
| | Region | SWNSGALTSGVHTFPAVLQSSGLYSLSSVVTVPSSSLGT | |
| | | QTYICNVNHKPSNTKVDKRVEPKSCDKTHTCPPCPAPE | |
| | | LLGGPSVFLFPPKPKDTLMISRTPEVTCVVVDVSHEDPE | |
| | | VKFNWYVDGVEVHNAKTKPREEQYNSTYRVVSVLTV | |
| | | LHQDWLNGKEYKCKVSNKALPAPIEKTISKAKGQPREP | |
| | | QVYTLPPSREEMTKNQVSLTCLVKGFYPSDIAVEWESN | |
| | | GQPENNYKTTPPVLDSDGSFFLYSKLTVDKSRWQQGN | |
| | | VFSCSVMHEALHNHYTQKSLSLSPG | |
| | LC Constant | RTVAAPSVFIFPPSDEQLKSGTASVVCLLNNFYPREAKV | 436 |
| | Region | QWKVDNALQSGNSQESVTEQDSKDSTYSLSSTLTLSKA | |
| | | DYEKHKVYACEVTHQGLSSPVTKSFNRGEC | |
| | Heavy Chain | QVQLVQSGAEVKKPGASVKVSCKASGYTFTGYYIHWV | 483 |
| | | RQAPGQGLEWMGWINPNSGGTNYAQKFQGRVTMTRD | |
| | | TSIGTAYMELSRLRSDDTAIVYCTREDTWNYIDYWGQG | |
| | | TLVTVSSASTKGPSVFPLAPSSKSTSGGTAALGCLVKDY | |
| | | FPEPVTVSWNSGALTSGVHTFPAVLQSSGLYSLSSVVTV | |
| | | PSSSLGTQTYICNVNHKPSNTKVDKRVEPKSCDKTHTCP | |
| | | PCPAPELLGGPSVFLFPPKPKDTLMISRTPEVTCVVVDVS | |
| | | HEDPEVKFNWYVDGVEVHNAKTKPREEQYNSTYRVVS | |
| | | VLTVLHQDWLNGKEYKCKVSNKALPAPIEKTISKAKGQ | |
| | | PREPQVYTLPPSREEMTKNQVSLTCLVKGFYPSDIAVEW | |
| | | ESNGQPENNYKTTPPVLDSDGSFFLYSKLTVDKSRWQQ | |
| | | GNVFSCSVMHEALHNHYTQKSLSLSPG | |
| | Light Chain | DIQMTQSPSSLSASVGDRVTITCRASQSISSHLNWYQQK | 484 |
| | | PGKAPKLLIYAASSLHSGVPSRFSGSGSGTDFTLTISSLQP | |
| | | EDFATYCCQQSYSTPRTFGQGTKVEIKRTVAAPSVFIFPP | |
| | | SDEQLKSGTASVVCLLNNFYPREAKVQWKVDNALQSG | |
| | | NSQESVTEQDSKDSTYSLSSTLTLSKADYEKHKVYACE | |
| | | VTHQGLSSPVTKSFNRGEC | |
| CD22- | HC CDR1 | TSGMCVS | 489 |
| mAb-2^{∧} | HC CDR2 | LIDWDGDNYYSTSLKT | 490 |
| | HC CDR3 | MGYTYGYDAFDI | 491 |
| | LC CDR1 | RASQSIGSLLH | 492 |
| | LC CDR2 | FASQSLS | 493 |
| | LC CDR3 | HQSRSLPFT | 494 |
| | VH | QVTLRESGPALVKPTQTLTLTCTFSGISLTTSGMCVSWI | 495 |
| | | RQPPGKALEWLALIDWDGDNYYSTSLKTRLTISKDTSK | |
| | | NQVVLTMASMDPVDTATYYCARMGYTYGYDAFDIW | |
| | | GQGTMVTVSS | |
| | VL | EIVLTQSPEFQSVTPTEKVTITCRASQSIGSLLHWYQQK | 496 |
| | | PDQSPKLLIKFASQSLSGVPSRFSGSGSGTDFTLTINSLEA | |
| | | EDAATYYCHQSRSLPFTFGPGTKVDIN | |
| | HC Constant | ASTKGPSVFPLAPSSKSTSGGTAALGCLVKDYFPEPVTV | 435 |
| | Region | SWNSGALTSGVHTFPAVLQSSGLYSLSSVVTVPSSSLGT | |
| | | QTYICNVNHKPSNTKVDKRVEPKSCDKTHTCPPCPAPEL | |
| | | LGGPSVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPIEKTISKAKGQPREPQV | |
| | | YTLPPSREEMTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| | LC Constant | RTVAAPSVFIFPPSDEQLKSGTASVVCLLNNFYPREAKV | 436 |
| | Region | QWKVDNALQSGNSQESVTEQDSKDSTYSLSSTLTLSKA | |
| | | DYEKHKVYACEVTHQGLSSPVTKSFNRGEC | |
| | Heavy Chain | QVTLRESGPALVKPTQTLTLTCTFSGISLTTSGMCVSWIR | 497 |
| | | QPPGKALEWLALIWDGDNYYSTSLKTRLTISKDTSKN | |
| | | QVVLTMASMDPVDTATYYCARMGYTYGYDAFDIWGQ | |
| | | GTMVTVSSASTKGPSVFPLAPSSKSTSGGTAALGCLVKD | |
| | | YFPEPVTVSWNSGALTSGVHTFPAVLQSSGLYSLSSVVT | |
| | | VPSSSLGTQTYICNVNHKPSNTKVDKRVEPKSCDKTHTC | |
| | | PPCPAPELLGGPSVFLFPPKPKDTLMISRTPEVTCVVVDV | |
| | | SHEDPEVKFNWYVDGVEVHNAKTKPREEQYNSTYRVV | |
| | | SVLTVLHQDWLNGKEYKCKVSNKALPAPIEKTISKAKG | |
| | | QPREPQVYTLPPSREEMTKNQVSLTCLVKGFYPSDIAVE | |
| | | WESNGQPENNYKTTPPVLDSDGSFFLYSKLTVDKSRWQ | |
| | | QGNVFSCSVMHEALHNHYTQKSLSLSPG | |
| | Light Chain | EIVLTQSPEFQSVTPTEKVTITCRASQSIGSLLHWYQQKP | 498 |
| | | DQSPKLLIKFASQSLSGVPSRFSGSGSGTDFTLTINSLEAE | |
| | | DAATYYCHQSRSLPFTFGPGTKVDINRTVAAPSVFIFPPS | |
| | | DEQLKSGTASVVCLLNNFYPREAKVQWKVDNALQSGN | |
| | | SQESVTEQDSKDSTYSLSSTLTLSKADYEKHKVYACEVT | |
| | | HQGLSSPVTKSFNRGEC | |
| CD22- | HC CDR1 | TSGMCVS | 489/503 |
| mAb-3^{∧} | HC CDR2 | LIDWDDDKYYSTSLKT | 504 |
| | HC CDR3 | IRYIYGYDYFDY | 505 |
| | LC CDR1 | RASQRISSQLN | 506 |
| | LC CDR2 | AASSLQS | 507 |
| | LC CDR3 | QQTYSKPIT | 508 |
| | VH | QVTLRESGPALVKTTQTLTLTCTFSGFSLSTSGMCVSWI | 509 |
| | | RQPPGKALEWLALIDWDDDKYYSTSLKTRLTISKDTSK | |
| | | SQVVLTMTSMDPVDTATYYCARIRYIYGYDYFDYWGQ | |
| | | GTLVTVSS | |
| | VL | DIQMTQSPSSLSASVGDRVTITCRASQRISSQLNWYQQK | 510 |
| | | PGKAPKLLIYAASSLQSGVPSRFSGSGSGTEFTLTISSLQP | |
| | | EDIATYCCQQTYSKPITFGQGTRLEIK | |
| | HC Constant | ASTKGPSVFPLAPSSKSTSGGTAALGCLVKDYFPEPVTV | 435 |
| | Region | SWNSGALTSGVHTFPAVLQSSGLYSLSSVVTVPSSSLGT | |
| | | QTYICNVNHKPSNTKVDKRVEPKSCDKTHTCPPCPAPEL | |
| | | LGGPSVFLFPPKPKDTLMISRTPEVTCVVVDVSHEDPEV | |
| | | KFNWYVDGVEVHNAKTKPREEQYNSTYRVVSVLTVLH | |
| | | QDWLNGKEYKCKVSNKALPAPIEKTISKAKGQPREPQV | |
| | | YTLPPSREEMTKNQVSLTCLVKGFYPSDIAVEWESNGQP | |
| | | ENNYKTTPPVLDSDGSFFLYSKLTVDKSRWQQGNVFSC | |
| | | SVMHEALHNHYTQKSLSLSPG | |
| | LC Constant | RTVAAPSVFIFPPSDEQLKSGTASVVCLLNNFYPREAKV | 436 |
| | Region | QWKVDNALQSGNSQESVTEQDSKDSTYSLSSTLTLSKA | |
| | | DYEKHKVYACEVTHQGLSSPVTKSFNRGEC | |
| | Heavy Chain | QVTLRESGPALVKTTQTLTLTCTFSGFSLSTSGMCVSWI | 511 |
| | | RQPPGKALEWLALIDWDDDKYYSTSLKTRLTISKDTSKS | |
| | | QVVLTMTSMDPVDTATYYCARIRYIYGYDYFDYWGQG | |
| | | TLVTVSSASTKGPSVFPLAPSSKSTSGGTAALGCLVKDY | |
| | | FPEPVTVSWNSGALTSGVHTFPAVLQSSGLYSLSSVVTV | |
| | | PSSSLGTQTYICNVNHKPSNTKVDKRVEPKSCDKTHTCP | |
| | | PCPAPELLGGPSVFLFPPKPKDTLMISRTPEVTCVVVDVS | |
| | | HEDPEVKFNWYVDGVEVHNAKTKPREEQYNSTYRVVS | |
| | | VLTVLHQDWLNGKEYKCKVSNKALPAPIEKTISKAKGQ | |
| | | PREPQVYTLPPSREEMTKNQVSLTCLVKGFYPSDIAVEW | |
| | | ESNGQPENNYKTTPPVLDSDGSFFLYSKLTVDKSRWQQ | |
| | | GNVFSCSVMHEALHNHYTQKSLSLSPG | |
| | Light Chain | DIQMTQSPSSLSASVGDRVTITCRASQRISSQLNWYQQ | 512 |
| | | KPGKAPKLLIYAASSLQSGVPSRFSGSGSGTEFTLTISSL | |
| | | QPEDIATYCCQQTYSKPITFGQGTRLEIKRTVAAPSVFIF | |
| | | PPSDEQLKSGTASVVCLLNNFYPREAKVQWKVDNALQ | |
| | | SGNSQESVTEQDSKDSTYSLSSTLTLSKADYEKHKVYA | |
| | | CEVTHQGLSSPVIKSFNRGEC | |
| CD22- | HC CDR1 | NYGMH | 443 |
| mAb-5^{∧} | HC CDR2 | VIYYDGSKNYYEDSVKG | 444 |
| | HC CDR3 | ELTGDAFDI | 429 |
| | LC CDR1 | RASQRIGSSLH | 445 |
| | LC CDR2 | YASQSFS | 431 |
| | LC CDR3 | HQSSSFPYT | 446 |
| | VH | QVQLVESGGDVVQPGRSLRLSCAASGFIFRNYGMHWV | 447 |
| | | RQAPGKGLEWVAVIYYDGSKNYYEDSVKGRFTISRDNSKN | |
| | | TLY | |
| | | LQMNSLRVEDTAVYYCARELTGDAFDIWGQGTMVTV | |
| | | SS | |
| | VL | EIVLTQSPDFQSLTPKEKVTITCRASQRIGSSLHWYQQK | 448 |
| | | PDQSPKLLIKYASQSFSGVPSRFSGSGSGTDFTLTINSLDA | |
| | | EDAATYYCHQSSSFPYTFGQGTKLQIK | |
| CD22- | HC CDR1 | SYGMH | 427 |
| mAb-6^{∧} | HC CDR2 | VIYYDGNKKYYVDSVKD | 449 |
| | HC CDR3 | ELTGDAFDI | 430 |
| | LC CDR1 | RASQRIGSSLH | 445 |
| | LC CDR2 | YASQSFS | 431 |
| | LC CDR3 | HQSSTFPYT | 450 |
| | VH | QVQLVESGGGVVQPGRSLRLSCAASGFTFSSYGMHWV | 451 |
| | | RQAPGKGLEWVAVIYYDGNKKYYVDSVKDRITISRDNS | |
| | | KNTLYLQMNSLRAEDTAVYYCARELTGDAFDIWGQGT | |
| | | MVTVSS | |
| | VL | EIVLTQSPDFQSVTPKEKVTITCRASQRIGSSLHWYQQKS | 452 |
| | | DQSPKLLIKYASQSFSGVPSRFSGSGSGTDFTLTINSLEAE | |
| | | DAATYYCHQSSTFPYTFGQGTKLEIK | |
| CD22- | HC CDR1 | SYGMH | 427 |
| mAb-7^{∧} | HC CDR2 | VIYYDGNNKYYADSVKG | 453 |
| | HC CDR3 | ELTGDAFDL | 454 |
| | LC CDR1 | RASQSIGSHLH | 455 |
| | LC CDR2 | YASQSFS | 431 |
| | LC CDR3 | HQSSRFPYT | 456 |
| | VH | QVQLVESGGGVVQPGRSLRLSCAASGFTFSSYGMHWV | 457 |
| | | RQTPGKGLEWVAVIYYDGNNKYYADSVKGRFTISRENS | |
| | | KNTLYLQMNSLRADDTALYYCARELTGDAFDLWGQGT | |
| | | MVTVSS | |
| | VL | EIVLTQSPDFQSATPKEKVTITCRASQSIGSHLHWYQQKP | 458 |
| | | DQSPKLLIKYASQSFSGVPSRFSGSGSGTDFTLTINRLED | |
| | | EDAATYYCHQSSRFPYTFGQGTKLDIK | |
| CD22-P49 | VH | QVQLVESGGGVVQPGRSLRLSCAASGFTFSSYGMHWV | 523 |
| (ProL) | | RQA | |
| | | PGKGLEWVAHIYYDGSKKYYADSVKGRFTISRDNSKNT | |
| | | LYLQMNSLRAEDTAVYYCARELTGDAFDIWGQGTKVT | |
| | | VSS | |
| | VL | EIVLTQTPSSLSASLGDRVTISCRASQSIGSSLHWYQQKP | 524 |
| | | DQSPKLLIKYASQSFSGVPSKFIGSGSGTDFTLTINSLEAE | |
| | | DAATFYCHQSSTLPYTFGQGTKLEIK | |
| CD22-P155 | VH | QVQLVQSGAEVKKPGASVKVSCKASGYTFTGYYIHWV | 525 |
| | | RQAPGQGLEWMGWINPNSGGTNYAQKFQGRVTMTRD | |
| | | TSIGTAYMELSRLRSDDTAIYYCTREDTWNYIDYWGQG | |
| | | TLVTVSS | |
| | VL | DIQMTQSPSSLSASVGDRVTITCRASQSISSHLNWYQQK | 526 |
| | | PGKAPKLLIYAASSLHSGVPSRFSGSGSGTDFTLTISSLQP | |
| | | EDFATYCCQQSYSTPRTFGQGTKVEIK | |
| CD22-P197 | VH | QVTLRESGPALVKPTQTLTLTCTFSGISLTTSGMCVSWIR | 527 |
| (ProL) | | QPPGKALEWLALIDWDGDNYYSTSLKTRLTISKDTSKN | |
| | | QVVLTMASMDPVDTATYYCARMGYTYGYDAFDIWGQ | |
| | | GTMVTVSS | |
| | VL | EIVLTQTPSSLSASLGDRVTISCRASQSIGSLLHWYQQKP | 528 |
| | | DQSPKLLIKFASQSLSGVPSRFSGSGSGTDFTLTINSLEAE | |
| | | DAATYYCHQSRSLPFTFGPGTKVDIN | |
| CD22-198 | VH | QVTLRESGPALVKTTQTLTLTCTFSGFSLSTSGMCVSWI | 529 |
| | | RQPPGKALEWLALIDWDDDKYYSTSLKTRLTISKDTSKS | |
| | | QVVLTMTSMDPVDTATYYCARI | |
| | | RYIYGYDYFDYWGQGTLVTVSS | |
| | VL | DIQMTQSPSSLSASVGDRVTITCRASQRISSQLNWYQQK | 530 |
| | | PGKAPKLLIYAASSLQSGVPSRFSGSGSGTEFTLTISSLQP | |
| | | EDIATYCCQQTYSKPITFGQGTRLEIK | |
| CD22-P12 | VH | QVQLVQSGAEVKKPGASVKVSCKASGYTFTGYYMHW | 531 |
| (ProL) | | VRQAPGQGLEWMGWVDPKNGDTNYAHKFQGRVTMI | |
| | | WDTSISTAYMELSRLISDDTAVYYCARDADFDYWGQG | |
| | | TLVTVSS | |
| | VL | DVVMTQTPSSLSASLGDRVTISCRASQSLVYIDGNTYLN | 532 |
| | | WFRQRPGQSPRRLIYKVSNRDYGVPDRFSGSGSGTDFTL | |
| | | KISRVEAEDVGVYYCMQGTHWPLTFGGGTKVEIK | |
| CD22-P12 | VH | QVQLVQSGAEVKKPGASVKVSCKASGYTFTGYYMHW | 531 |
| | | VRQAPGQGLEWMGWVDPKNGDTNYAHKFQGRVTMI | |
| | | WDTSISTAYMELSRLISDDTAVYYCARDADFDYWGQG | |
| | | TLVTVSS | |
| | VL | DVVMTQSPLSLPVTLGQPASISCRSSQSLVYIDGNTYLNW | 533 |
| | | FRQRPGQSPRRLIYKVSNRDYGVPDRFSGSGSGTDFTLKI | |
| | | SRVEAEDVGVYYCMQGTHWPLTFGGGTKVEIK | |
| CD22-P49 | VH | QVQLVESGGGVVQPGRSLRLSCAASGFTFSSYGMHWV | 523 |
| | | RQAPGKGLEWVAIIYYDGSKKYYADSVKGRFTISRDNS | |
| | | KNTLYLQMNSLRAEDTAVYYCARELTGDAFDIWGQGT | |
| | | KVTVSS | |
| | VL | EIVLTQSPDFQSVTPKEKVTITCRASQSIGSSLHWYQQKPD | 534 |
| | | QSPKLLIKYASQSFSGVPSKFIGSGSGTDFTLTINSLEAEDA | |
| | | ATFYCHQSSTLPYTFGQGTKLEIK | |
| CD22-P197 | VH | QVTLRESGPALVKPTQTLTLTCTFSGISLTTSGMCVSWIR | 527 |
| | | QPPGKALEWLALIDWDGDNYYSTSLKTRLTISKDTSKN | |
| | | QVVLTMASMDPVDTATYYCARMGYTYGYDAFDIWGQ | |
| | | GTMVTVSS | |
| | VL | EIVLTQSPEFQSVTPTEKVTITCRASQSIGSLLHWYQQKPD | 535 |
| | | QSPKLLIKFASQSLSGVPSRFSGSGSGTDFTLTINSLEAED | |
| | | AATYYCHQSRSLPFTFGPGTKVDIN | |
| M971 | VH | QVQLQQSGPGLVKPSQTLSLTCAISGDSVSSNSAAWNWI | 536 |
| | | RQSPSRGLEWLGRTYYRSKWYNDYAVSVKSRITINPDT | |
| | | SKNQFSLQLNSVTPEDTAVYYCAREVTGDLEDAFDIWG | |
| | | QGTMVTVSS | |
| | VL | DIQMTQSPSSLSASVGDRVTITCRASQTIWSYLNWYQQR | 537 |
| | | PGKAPNLLIYAASSLQSGVPSRFSGRGSGTDFTLTISSLQ | |
| | | AEDFATYYCQQSYSIPQTFGQGTKLEIK | |

In some embodiments, one or more modifications to the VH and VL domains as described herein were made while not substantially impacting the binding affinity of the CD22 antibodies.

In some embodiments, the heavy chain variable region and the light chain variable region are connected through a linker sequence (Linker A), such as a linker in Table B. In some embodiments, the linker comprises (GGGGS) n. In some embodiments, n is equal to 4 (SEQ ID NO: 344).

In some embodiments, the heavy chain variable region comprises a lead peptide sequence at the N-terminus before the CD22 VH region. In some embodiments, the heavy chain variable region comprises a signal peptide, such as a signal peptide derived from CD8α. In some embodiments, the signal peptide comprises MALPVTALLLPLALLLHAARP (SEQ ID NO: 515, CD8α signal peptide) or MDMRVPAQLLGLLLLWLPGAKC (SEQ ID NO: 520).

In some embodiments, the VL region and the Hinge region is connected through a linker sequence (Linker B). In some embodiments, Linker B comprises (AAA) n. In some embodiments, Linker B is AAA. In some embodiments, Linker B comprises AS. In some embodiments, Linker B is AS.

In some embodiments, the Hinge-transmembrane region is about 5 to 10 aa long. In some embodiments, the Hinge-transmembrane region is about 10 to 20 aa long. In some embodiments, the Hinge-transmembrane region is about 20 to 30 aa long. In some embodiments, the Hinge-transmembrane region is about 30 to 40 aa long. In some embodiments, the Hinge-transmembrane region is about 40 to 50 aa long. In some embodiments, the Hinge-transmembrane region is about 50 to 60 aa long. In some embodiments, the Hinge-transmembrane region is about 60 to 70 aa long. In some embodiments, the Hinge-transmembrane region is about 70 to 80 aa long. In some embodiments, the Hinge-transmembrane region is about 80 to 90 aa long. In some embodiments, the Hinge-transmembrane region is about 90 to 100 aa long. In some embodiments, the Hinge-transmembrane region is about 100 to 120 aa long.

In some embodiments, the Hinge—transmembrane region is derived from human CD8alpha or human CD28 (see Cells 2020, 9, 1182; doi: 10.3390/cells9051182).

In some embodiments, the Hinge—transmembrane region is derived from human CD8alpha, the full sequence or a functional comprising fragment of LSNSIMYFSHFVPVFLPAKPTTTPAPRPPTPAPTIASQPLSLRPEACRPAAGGAVHTRGLD FACDIYIWAPLAGTCGVLLLSLVITLYCNHRN (SEQ ID NO: 521), such as about 10-20 amino acids, about 20-30 amino acids, about 30-40 amino acids, about 40-50 amino acids, about 50-60 amino acids, about 60-70 amino acids, about 70−80 amino acids, or about 80-90 amino acids of SEQ ID NO: 521. In some embodiments, the Hinge-transmembrane region comprises or is SEQ ID NO: 521, or share at least 90%, 91%, 92%, 93%, 94%, 95%, 96%, 97%, 98%, 99%, 99.5% with SEQ ID NO: 521. In some embodiments, the Hinge-transmembrane region comprises or is SEQ ID NO: 538, or share at least 90%, 91%, 92%, 93%, 94%, 95%, 96%, 97%, 98%, 99%, 99.5% with SEQ ID NO: 538. In some embodiments, the Hinge-transmembrane region comprises or is SEQ ID NO: 539, or share at least 90%, 91%, 92%, 93%, 94%, 95%, 96%, 97%, 98%, 99%, 99.5% with SEQ ID NO: 539. In some embodiments, the Hinge-transmembrane region comprises or is SEQ ID NO: 540, or share at least 90%, 91%, 92%, 93%, 94%, 95%, 96%, 97%, 98%, 99%, 99.5% with SEQ ID NO: 540.

In some embodiments, the Hinge-transmembrane region is derived from human CD28, comprising the full sequence or a functional fragment of IEVMYPPPYLDNERSNGTIIHVKGKHLCPSPLFPGPSKPFWVLVVVGGVLACYSLLVTVA FIIFWV (SEQ ID NO: 522), such as about 10-20 amino acids, about 20-30 amino acids, about 30-40 amino acids, about 40-50 amino acids, about 50-60 amino acids, or about 60-70 amino acids of SEQ ID NO: 522. In some embodiments, the Hinge-transmembrane region comprises or is SEQ ID NO: 522. In some embodiments, the Hinge region comprises or is SEQ ID NO: 541. In some embodiments, the transmembrane region comprises or is SEQ ID NO: 542, or share at least 90%, 91%, 92%, 93%, 94%, 95%, 96%, 97%, 98%, 99%, 99.5% with SEQ ID NO: 542.

In some embodiments, the Co-stimulatory (co-stim) domain is derived from CD28. In some embodiments, the co-stim domain comprises the full sequence or a functional fragment of RSKRSRLLHSDYMNMTPRRPGPTRKHYQPYAPPRDFAAYRS (SEQ ID NO: 543). In some embodiments, the transmembrane region comprises or is SEQ ID NO: 543, or share at least 90%, 91%, 92%, 93%, 94%, 95%, 96%, 97%, 98%, 99%, 99.5% with SEQ ID NO: 543.

In some embodiments, the Signaling domain is derived from CD3 zeta. In some embodiments, the Signaling domain comprises the full sequence or a functional fragment of RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 544). In some embodiments, the transmembrane region comprises or is SEQ ID NO: 544, or share at least 90%, 91%, 92%, 93%, 94%, 95%, 96%, 97%, 98%, 99%, 99.5% with SEQ ID NO: 544.

In some embodiments, the payload comprises an mRNA selected from any one from the table below, or an mRNA encoding an amino acid sequence selected from any one from the table below:

TABLE E

| Payload Name | Amino acids | Nucleotides |
|---|---|---|
| P49 with CD28 hinge | SEQ ID NO: 545 | SEQ ID NO: 46 |
| P49 with short CD8 hinge | SEQ ID NO: 546 | SEQ ID NO: 47 |
| P49 with intermediate CD8 hinge | SEQ ID NO: 547 | SEQ ID NO: 48 |
| P49 with long CD8 hinge | SEQ ID NO: 548 | SEQ ID NO: 49 |
| P155 with CD28 hinge | SEQ ID NO: 549 | SEQ ID NO: 50 |
| P155 with short CD8 hinge | SEQ ID NO: 550 | SEQ ID NO: 51 |
| P155 with intermediate CD8 hinge | SEQ ID NO: 551 | SEQ ID NO: 52 |
| P155 with long CD8 hinge | SEQ ID NO: 552 | SEQ ID NO: 53 |
| P197 with CD28 hinge | SEQ ID NO: 553 | SEQ ID NO: 54 |
| P197 with short CD8 hinge | SEQ ID NO: 554 | SEQ ID NO: 55 |
| P197 with intermediate CD8 hinge | SEQ ID NO: 555 | SEQ ID NO: 56 |
| P197 with long CD8 hinge | SEQ ID NO: 556 | SEQ ID NO: 57 |
| P198 with CD28 hinge | SEQ ID NO: 557 | SEQ ID NO: 58 |
| P198 with short CD8 hinge | SEQ ID NO: 558 | SEQ ID NO: 59 |
| P198 with intermediate CD8 hinge | SEQ ID NO: 559 | SEQ ID NO: 60 |
| P198 with long CD8 hinge | SEQ ID NO: 560 | SEQ ID NO: 61 |
| m971 with CD28 hinge | SEQ ID NO: 561 | SEQ ID NO: 62 |
| m971 with short CD8 hinge | SEQ ID NO: 562 | SEQ ID NO: 63 |
| m971 with intermediate CD8 hinge | SEQ ID NO: 563 | SEQ ID NO: 64 |
| m971 with long CD8 hinge | SEQ ID NO: 564 | SEQ ID NO: 65 |
| mkap-CD22-P49-CD28-40 (TTR-034) | SEQ ID NO: 97 | SEQ ID NO: 86 |
| mkap-CD22-P155-CD28-40 (TRR-035) | SEQ ID NO: 98 | SEQ ID NO: 87 |
| mkap-CD22-P155-CD8-46 (TRR-036) | SEQ ID NO: 99 | SEQ ID NO: 88 |
| mkap-CD22-P197-CD8-26 (TRR-039) | SEQ ID NO: 100 | SEQ ID NO: 89 |
| mkap-CD22-P197-CD8-46 (TRR-040) | SEQ ID NO: 101 | SEQ ID NO: 90 |
| CD8a-CD22-P197-CD8-46 (TRR-041) | SEQ ID NO: 102 | SEQ ID NO: 91 |
| mkap-CD22-P198-CD28-40 (TRR-042) | SEQ ID NO: 103 | SEQ ID NO: 92 |
| mkap-CD22-M2-CD28-40 (TRR-043) | SEQ ID NO: 104 | SEQ ID NO: 93 |
| mkap-CD22-m971-CD8-46 (TRR-044) | SEQ ID NO: 105 | SEQ ID NO: 94 |
| mkap-CD22-m971-CD8-66 (TRR-045) | SEQ ID NO: 106 | SEQ ID NO: 95 |
| mkap-CD22-m971DB-CD8-46 (TRR-046) | SEQ ID NO: 107 | SEQ ID NO: 96 |
| CD8a-CD22-P49-CD28-40 (TRR-102) | SEQ ID NO: 116 | SEQ ID NO: 108 |
| CD8a-CD22-P155-CD28-40 (TRR-103) | SEQ ID NO: 117 | SEQ ID NO: 109 |
| CD8a-CD22-P197-CD8-26 (TRR-105) | SEQ ID NO: 118 | SEQ ID NO: 110 |
| CD8a-CD22-P198-CD28-40 (TRR-106) | SEQ ID NO: 119 | SEQ ID NO: 111 |
| CD8a-CD22-M2-CD28-40 (TRR-107) | SEQ ID NO: 120 | SEQ ID NO: 112 |
| CD8a-CD22-m971DB-CD8-46 (TRR-108) | SEQ ID NO: 121 | SEQ ID NO: 113 |
| CD8a-CD19-FMC63-CD28-40 (TRR-110) | SEQ ID NO: 122 | SEQ ID NO: 114 |
| CD8a-CD22-P49-CD28-40-CD137 (TRR-121) | SEQ ID NO: 123 | SEQ ID NO: 115 |
| CD8a-CD22-P49-CD28-40 (CoE UTRs; MRT14577) | SEQ ID NO: 126 | SEQ ID NO: 124 |
| CD8a-CD22-P49-CD28-40-CD137 (CoE UTRs; MRT14580) | SEQ ID NO: 127 | SEQ ID NO: 125 |
| TTR-034 full mRNA | SEQ ID NO: 97 | SEQ ID NO: 128 |
| TTR-035 full mRNA | SEQ ID NO: 98 | SEQ ID NO: 129 |
| TTR-036 full mRNA | SEQ ID NO: 99 | SEQ ID NO: 130 |
| TTR-039 full mRNA | SEQ ID NO: 100 | SEQ ID NO: 131 |
| TTR-040 full mRNA | SEQ ID NO: 101 | SEQ ID NO: 132 |
| TTR-041 full mRNA | SEQ ID NO: 102 | SEQ ID NO: 133 |
| TTR-042 full mRNA | SEQ ID NO: 103 | SEQ ID NO: 134 |
| TTR-043 full mRNA | SEQ ID NO: 104 | SEQ ID NO: 135 |
| TTR-044 full mRNA | SEQ ID NO: 105 | SEQ ID NO: 136 |
| TTR-045 full mRNA | SEQ ID NO: 106 | SEQ ID NO: 137 |
| TTR-046 full mRNA | SEQ ID NO: 107 | SEQ ID NO: 138 |
| TTR-102 full mRNA | SEQ ID NO: 116 | SEQ ID NO: 139 |
| TTR-103 full mRNA | SEQ ID NO: 117 | SEQ ID NO: 140 |
| TTR-105 full mRNA | SEQ ID NO: 118 | SEQ ID NO: 141 |
| TTR-106 full mRNA | SEQ ID NO: 119 | SEQ ID NO: 142 |
| TTR-107 full mRNA | SEQ ID NO: 120 | SEQ ID NO: 143 |
| TTR-108 full mRNA | SEQ ID NO: 121 | SEQ ID NO: 144 |
| TTR-110 full mRNA | SEQ ID NO: 122 | SEQ ID NO: 145 |
| TTR-121 full mRNA | SEQ ID NO: 123 | SEQ ID NO: 146 |
| MRT14577 full mRNA | SEQ ID NO: 126 | SEQ ID NO: 147 |
| MRT14580 full mRNA | SEQ ID NO: 127 | SEQ ID NO: 148 |

In some embodiments, a nucleotide sequence in Table E further comprises a 5′ UTR comprising SEQ ID NO: 516 or SEQ ID NO: 518. In some embodiments, the nucleotide sequence further comprises a 3′ UTR comprising SEQ ID NO: 517 or SEQ ID NO: 519. In some embodiments, the 5′ UTR comprises SEQ ID NO: 516 and the 3′ UTR comprises SEQ ID NO: 517. In some embodiments, the 5′ UTR comprises SEQ ID NO: 518 and the 3′ UTR comprises SEQ ID NO: 519.

In some embodiments, a nucleotide sequence in Table E further comprises a 5′ Cap. In some embodiments, the 5′ Cap is Cap 1. In some embodiments, a nucleotide sequence in Table E comprises Cap 1, a 5′ UTR comprising SEQ ID NO: 516, and a 3′ UTR comprises SEQ ID NO: 517. In some embodiments, a nucleotide sequence in Table E comprises Cap 1, a 5′ UTR comprising SEQ ID NO: 518, and the 3′ UTR comprises SEQ ID NO: 519.

In some embodiments, the payload mRNA comprises a nucleotide sequence encoding a payload polypeptide comprising or consisting of SEQ ID NO: 116, or a payload polypeptide having at least 90%, 91%, 92%, 93%, 94%, 95%, 96%, 97%, 98%, 99%, 99.5% identity to SEQ ID NO: 116. In some embodiments, such a nucleotide sequence comprises SEQ ID NO: 108, or a nucleotide sequence having at least 90%, 91%, 92%, 93%, 94%, 95%, 96%, 97%, 98%, 99%, 99.5% identity to SEQ ID NO: 108, or a nucleotide sequence that hybridizes to a nucleic acid comprising the complement of SEQ ID NO: 108 under stringent conditions, such as i) washing at 50° C. with 0.015 M NaCl, 0.0015 M sodium citrate and 0.1% SDS; or ii) washing at 42° C. in 0.2%×SSC and 0.1% SDS. In some embodiments, the payload comprises or is SEQ ID NO: 139 (TTR-102).

In some embodiments, the payload mRNA comprises a nucleotide sequence encoding a payload polypeptide comprising or consisting of SEQ ID NO: 126, or a payload polypeptide having at least 90%, 91%, 92%, 93%, 94%, 95%, 96%, 97%, 98%, 99%, 99.5% identity to SEQ ID NO: 126. In some embodiments, such a nucleotide sequence comprises SEQ ID NO: 127, or a nucleotide sequence having at least 90%, 91%, 92%, 93%, 94%, 95%, 96%, 97%, 98%, 99%, 99.5% identity to SEQ ID NO: 127, or a nucleotide sequence that hybridizes to a nucleic acid comprising the complement of SEQ ID NO: 127 under stringent conditions, such as i) washing at 50° C. with 0.015 M NaCl, 0.0015 M sodium citrate and 0.1% SDS; or ii) washing at 42° C. in 0.2%×SSC and 0.1% SDS. In some embodiments, the payload comprises or is SEQ ID NO: 147 (MRT14577).

In some embodiments, the payload RNA in the LNP is an in vitro transcribed (IVT) RNA. For example, the RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. In some embodiments, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. In some embodiments, 3′ UTRs can be selected or designed to increase the stability of the transcribed RNA. In some embodiments, the mRNA has both a cap on the 5′ end and a 3′ poly(A) tail (PolyA tail) which determine ribosome binding, initiation of translation and stability mRNA in the cell. The template DNA can be a circular DNA template or a linear DNA template. In some embodiments, poly(A) sequence is integrated into the RNA. In some embodiments, the poly (A) tail is arranged from about 100 nucleotides to between 100 and 200 nucleotides, between 200 and 300 nucleotides, or between 300 and 400 nucleotides. In some embodiments, the poly (A) tail comprises or is SEQ ID NO: 45.

In some embodiments, the RNA comprises one or more modified nucleosides. In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Kariko et ah, 2008, Mol Ther 16:1833-1840; Anderson et ah, 2010, Nucleic Acids Res 38:5884-5892; Anderson et al., 2011, Nucleic Acids Research 39:9329-9338; Kariko et al., 2011, Nucleic Acids Research 39: el42; Kariko et al., 2012, Mol Ther 20:948-953; Kariko et al., 2005, Immunity 23:165-175). In some embodiments, the RNA comprises pseudouridines (e.g., 1-methyl-pseudouridines). In some embodiments, the modified nucleoside is mlacp,P (1-methyl-3-(3-amino-3-carboxypropyl) pseudouridine. In another embodiment, the modified nucleoside is m1xP (1-methylpseudouridine). In another embodiment, the modified nucleoside is *Pm (2′-O-methylpseudouridine). In another embodiment, the modified nucleoside is m5D (5-methyldihydrouridine). In another embodiment, the modified nucleoside is m3vP (3-methylpseudouridine). In another embodiment, the modified nucleoside is a pseudouridine moiety that is not further modified (*P). In another embodiment, the modified nucleoside is a monophosphate, diphosphate, or triphosphate of any of the above pseudouridines. In another embodiment, the modified nucleoside is any other pseudouridine-like nucleoside known in the art. Pseudouridines described herein can be incorporated into RNA molecules through methods known in the art, such as in vitro transcription.

In another embodiment, the nucleoside that is modified in the nucleoside-modified RNA is a modified uridine (U). In another embodiment, the modified nucleoside is a modified cytidine (C). In another embodiment, the modified nucleoside is a modified adenosine (A). In another embodiment, the modified nucleoside is a modified guanosine (G).

In another embodiment, the modified nucleoside of the present invention is m5C (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is ′P (pseudouridine). In another embodiment, the modified nucleoside is Um (2′-O-methyluridine).

In other embodiments, the modified nucleoside is m1A (1-methyladenosine); m2A (2-methyladenosine); Am (2′-O-methyladenosine); ms2m6A (2-methylthio-N6-methyladenosine); 16A (N6-isopentenyladenosine); ms216A (2-methylthio-N6-isopentenyladenosine); io6A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonylcarbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyladenosine); ms2hn6A (2-methylthio-N6-hydroxynorvalyl carbamoyladenosine); Ar (p) (2′-O-ribosyladenosine (phosphate)); I (inosine); m1! (1-methylinosine); m′lm (1,2′-O-dimethylinosine); m3C (3-methylcytidine); Cm (2′-O-methylcytidine); s2C (2-thiocytidine); ac4C (N4-acetylcytidine); PC (5-formylcytidine); m5Cm (5,2′-O-dimethylcytidine); ac4Cm (N4-acetyl-2′-O-methylcytidine); k2C (lysidine); m′G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); 02yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQo (7-cyano-7-deazaguanosine); preQi (7-aminomethyl-7-deazaguanosme); G+(archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxy carbonylmethyluridine); mcm5Um (5-methoxycarbonylmethyl-2′-O-methyluridine); mcm5s2U (5-methoxycarbonylmethyl-2-thiouridine); nm5s2U (5-aminomethyl-2-thiouridine); mnm5U (5-methylaminomethyluridine); mnmVU (5-methylaminomethyl-2-thiouridine); mnm5se2U (5-methylaminomethyl-2-selenouridine); ncm5U (5-carbamoylmethyluridine); ncm5Um (5-carbamoylmethyl-2′-O-methyluridine); cmnm5U (5-carboxymethylaminomethyluridine); cmnm5Um (5-carboxymethylaminomethyl-21-O-methyluridine); cmnm5s2U (5-carboxymethylaminomethyl-2-thiouridine); m A (N6,N6-dimethyladenosine); Im (2′-O-methylinosine); m4C (N4-methylcytidine); m4Cm (N4,2′-O-dimethylcytidine); hm5C (5-hydroxymethylcytidine); m3U (3-methyluridine); cm5U (5-carboxymethyluridine); m6Am (N6,2′-O-dimethyladenosine); m62Am (N6,N6,O-2′-trimethyladenosine); m2 7G (N2,7-dimethylguanosine); m2 2 7G (N2,N2,7-trimethylguanosine); m3Um (3,2′-O-dimethyluridine); m5D (5-methyldihydrouridine); PCm (5-formyl-2′-O-methylcytidine); m′Gm (1,2′-O-dimethylguanosine); m′Am (1,2′-O-dimethyladenosine); Tm5U (5-taurinomethyluridine); rm5s2U (5-taurinomethyl-2-thiouridine)); imG-14 (4-demethylwyosine); imG2 (isowyosine); or ac6A (N6-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In various embodiments, between 0.1% and 100% of the residues in the nucleoside-modified of the present invention are modified (e.g., either by the presence of pseudouridine or another modified nucleoside base). In some embodiments, the fraction of modified residues is 0.1%. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.7%. In another embodiment, the fraction is 0.8%. In another embodiment, the fraction is 0.9%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 7%. In another embodiment, the fraction is 8%. In another embodiment, the fraction is 9%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 55%. In another embodiment, the fraction is 60%. In another embodiment, the fraction is 65%. In another embodiment, the fraction is 70%. In another embodiment, the fraction is 75%. In another embodiment, the fraction is 80%. In another embodiment, the fraction is 85%. In another embodiment, the fraction is 90%. In another embodiment, the fraction is 91%. In another embodiment, the fraction is 92%. In another embodiment, the fraction is 93%. In another embodiment, the fraction is 94%. In another embodiment, the fraction is 95%. In another embodiment, the fraction is 96%. In another embodiment, the fraction is 97%. In another embodiment, the fraction is 98%. In another embodiment, the fraction is 99%. In another embodiment, the fraction is 100%.

In some embodiments, the nucleic acid comprises pseudouridine. In some embodiments, the pseudouridine is N1-methyl-pseudouridine. In some embodiments, all uridines in an mRNA sequence of the present disclosure are replaced with N1-methyl-pseudouridine.

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, tRNA, microRNA, siRNA, gRNA (guide RNA), circRNA (circular RNA), ribozymes, decoy RNA or dicer substrate siRNA. It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may contain a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90% or, or greater than 90%.

In some embodiments, LNPs comprising an ionizable cationic lipid described herein, prepared and characterized using methods described herein, exhibit encapsulation efficiency of greater than 50, 55, 60, 65, 70, 75, 80, 82.5, 85, 87.5, 90, 92.5, 95, 97.5, or 99%. In some embodiments, LNPs comprising an ionizable cationic lipid described herein, prepared and characterized using methods described herein, exhibit encapsulation efficiency of greater than 87.5%. In some embodiments, LNPs comprising an ionizable cationic lipid described herein, prepared and characterized using methods described herein, exhibit dye accessible RNA of less than 50, 45, 40, 35, 30, 25, 20, 17.5, 15, 12.5, 10, 7.5, 5, 2.5, or 1%. In some embodiments, LNPs comprising an ionizable cationic lipid described herein, prepared and characterized using methods described herein, exhibit dye accessible RNA of less than 12.5%.

In some embodiments, LNPs comprising an ionizable cationic lipid described herein, prepared and characterized using methods described herein, exhibit total mRNA recovery of greater than 50, 55, 60, 65, 70, 75, 80, 85, 90, or 95%. In some embodiments, LNPs comprising an ionizable cationic lipid described herein, prepared and characterized using methods described herein, exhibit total mRNA recovery of greater than 80%.

In certain embodiments, the RNA payload is an mRNA, tRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In certain embodiments, one or more or all uridine bases may be N1-methylpseudouridines.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, the polyA sequence comprises about 50 to 200 adenine nucleotides, such as about 50, 60, 70, 80, 90, 100, 110, 120, 130, 140, 150, 160, 170, 180, 190, 200 adenine nucleotides. In some embodiments, the polyA sequence is SEQ ID NO: 45.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy) or fluorescence spectroscopy (e.g., with detection dyes).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy) or fluorescence spectroscopy (e.g., with detection dyes).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution containing the lipid composition before and after breaking up the LNP composition with one or more organic solvents or detergents. Fluorescence may be used to measure the amount of free mRNA in a solution. For the LNP compositions of the present disclosure, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### D. Other Lipid Components

In some embodiments, an LNP of the present disclosure comprises one or more structural lipids, one or more neutral phospholipids, or one or more free PEG-lipids, or any combination thereof.

### (i) Structural Lipid

In some embodiments, the structural lipid is sterol. In some embodiments, the structural lipid is cholesterol.

In certain embodiments, the LNP may comprise a sterol component, for example, one or more sterols selected from the group consisting of cholesterol, fecosterol, sitosterol, tomatidine, tomatine, ursolic acid, alpha-tocopherol, β-sitosterol, ergosterol, campesterol, stigmasterol, stigmastanol, brassicasterol, and mixtures thereof. In certain embodiments, the sterol is cholesterol. In some embodiments, the structural lipid includes cholesterol and a corticosteroid (such as prednisolone, dexamethasone, prednisone, and hydrocortisone), or a combination thereof.

The sterol (e.g., cholesterol) may be present in the LNP in a range of 20-70 mol %, 20-60 mol %, 20-50 mol %, 30-70 mol %, 30-60 mol %, 30-50 mol %, 40-70 mol %, 40-60 mol %, 40-50 mol %, 50-70 mol %, 50-60 mol %, or about 20 mol %, about 25 mol %, about 30 mol %, about 35 mol %, about 40 mol %, about 45 mol %, about 50 mol %, about 55 mol %, about 60 mol %, or about 65 mol %. In some embodiments, the sterol (e.g., cholesterol) may be present in the LNP in a range of about 25 mol % to about 45 mol %, such as about 30% to about 40%. In some embodiments, the sterol (e.g., cholesterol) may be present in the LNP about 28 mol % to 30 mol %, such as 29 mol % to about 30 mol % or such as about 29 mol %. In some embodiments, the sterol (e.g., cholesterol) may be present in the LNP about 38 mol % to 40 mol %, such as about 39 mol %. In some embodiments, the cholesterol is present in the LNP at a concentration of about 29 mol % when the DSPC is present in the LNP at a concentration of about 20 mol %. In some embodiments, the cholesterol is present in the LNP at a concentration of about 39 mol % when the DSPC is present in the LNP at a concentration of about 9.8 mol % or about 10 mol %.

### (ii) Neutral Phospholipid

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), 1,2-dilinoleoyl-sn-glycero-3-phosphocholine (DLPC), 1,2-dimyristoyl-sn-glycero-phosphocholine (DMPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, the neutral phospholipid is DSPC.

In certain embodiments, the LNP may contain one or more neutral phospholipids. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), and 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyrstoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, and sphingomyelin.

In some embodiments, the neutral phospholipid (e.g., DSPC) is present in the LNP in a range of 1-30 mol %, 1-15 mol %, 1-12 mol %, 1-10 mol %, 3-15 mol %, 3-12 mol %, 3-10 mol %, 4-15 mol %, 4-12 mol %, 4-10 mol %, 4-8 mol %, 5-15 mol %, 5-12 mol %, 5-10 mol %, 6-15 mol %, 6-12 mol %, 6-10 more percent, or about 1 mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, or about 15 mol %, about 16 mol %, about 17 mol %, about 18 mol %, about 19 mol %, about 20 mol %, about 21 mol %, about 22 mol %, about 23 mol %, about 24 mol %, or about 15 mol %, about 26 mol %, about 27 mol %, about 28 mol %, about 29 mol %, about 30 mol %. In some embodiments, the neutral phospholipid (e.g., DSPC) is present in the LNP in a range of about 19 mol % to about 21 mol %, such as about 20 mol %.

(iii) Free PEG-Lipid

In some embodiments, the free PEG-lipid is a stabilizing lipid. In some embodiments, the free PEG-lipid is selected d from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the free PEG-lipid is PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]—N,N-ditetradecylacetamide, diacylphosphatidylethanolamine comprising Dipalmitoyl (C16) chain or Distearoyl (C18) chain, or a PEG-distearoyl-phosphatidylethanolamine (PEG-DSPE) lipid. In some embodiments, the PEG-DAG comprises PEG-DMG, PEG-DPG, or PEG-DSG, or any combination thereof. In some embodiments, the free PEG-lipid comprises PEG-DPG. In some embodiments, the PEG-DPG is PEG 2000-DPG.

In some embodiments, the LNP may include one or more PEG or PEG-modified lipids. Such species may be alternately referred to as PEGylated lipids. A PEG lipid is a lipid modified with polyethylene glycol. As noted above, free PEG-lipids can be included in the LNP to reduce or eliminate non-specific binding via a targeting group when a lipid-immune cell targeting group is included in the LNP.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]—N,N-ditetradecylacetamide, or a PEG-distearoyl-phosphatidylethanolamine (PEG-DSPE) lipid.

In certain embodiments, the blend may contain a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the LNP in a range of 1-10 mol %, 1-8 mol %, 1-7 mol %, 1-6 mol %, 1-5 mol %, 1-4 mol %, 1-3 mol %, 2-8 mol %, 2-7 mol %, 2-6 mol %, 2-5 mol %, 2-4 mol %, 2-3 mol %, or about 1 mol %, about 2 mol %, about 3 mol %, about 4 mol %, or about 5 mol %. In some embodiments, the PEG-lipid is a free PEG-lipid.

In some embodiments, the PEG-lipid (e.g., PEG-DPG, such as PEG2.0K-DPG) may be present in the LNP in the range of 0.01-10 mol %, 0.01-5 mol %, 0.01-4 mol %, 0.01-3 mol %, 0.01-2 mol %, 0.01-1 mol %, 0.1-10 mol %, 0.1-5 mol %, 0.1-4 mol %, 0.1-3 mol %, 0.1-2 mol %, 0.1-1 mol %, 0.5-10 mol %, 0.5-5 mol %, 0.5-4 mol %, 0.5-3 mol %, 0.5-2 mol %, 0.5-1 mol %, 1-2 mol %, 3-4 mol %, 4-5 mol %, 5-6 mol %, or 1.25-1.75 mol %. In some embodiments, the PEG-lipid may be about 0.5 mol %, about 1 mol %, about 1.5 mol %, about 2 mol %, about 2.5 mol %, about 3 mol %, about 3.5 mol %, about 4 mol %, about 4.5 mol %, about 5 mol %, or about 5.5 mol % of the LNP. In some embodiments, the PEG-lipid (e.g., PEG-DPG, such as PEG2.0K-DPG) may be present in the LNP in about 1.2 to 1.8 mol %, such as about 1.5-1.6 mol %. In some embodiments, the PEG-lipid is a free PEG-lipid, such as PEG-DPG (e.g., PEG2.0K-DPG).

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back-bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an immune cell targeting group, and a PEG-lipid that is conjugated to immune cell targeting group. In some embodiments, the free PEG-lipid comprises the same or a different lipid as the lipid in the lipid-immune cell targeting group conjugate.

### E. LNP Compositions

In some aspect, provided herein are LNP compositions. In some embodiments, the LNP composition comprises a lipid-immune cell targeting group conjugate, an ionizable cationic lipid, a payload, a structural lipid, a neutral phospholipid, or a free PEG-lipid, or any combination thereof. In some embodiments, the LNP composition comprise mRNA, Lipid 15, PEG 2000-DPG (same as DPG-PEG 2K), DSPC, cholesterol, and DSPE-PEG 3.4K-anti CD8 VHH conjugate. In some embodiments, the LNP composition comprises mRNA, Lipid 15, PEG 2000-DPG (same as DPG-PEG 2K), DSPC, cholesterol, and DSPE-PEG 3.4K-anti CD8 VHH conjugate. In some embodiments, the LNP is described in FIG. 71. In some embodiments, the LNP is described in the Table F-1 or Table F-2 below.

TABLE F-1
Exemplary LNP formulation (Mol percent, excluding payload content)

| | | Mol % in the | Mol % in the |
|---|---|---|---|
| | | LNP or in the | LNP or in the |
| | | LNP | LNP |
| | | composition | composition |
| Components | Description | (Formulation 1) | (Formulation 2) |
| Ionizable cationic | (i) ionizable cationic Lipid 15 having the structure below, | about 48% to about | about 48% to |
| lipid | | 49.5% | about 49.5% |
| | 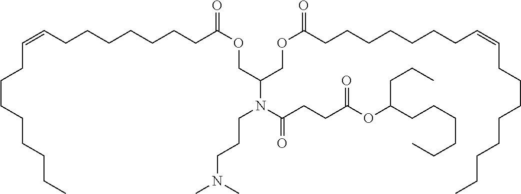 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | | |
| | Lipid 15 | | |
| | and/or a | | |
| | salt thereof; | | |
| Structural lipid | (ii) cholesterol; | about 39% to about | about 29% to |
| | | 40% | about 30% |
| Neutral lipid | (iii) DSPC; | about 9% to about | about 19% to |
| | | 11% | about 21% |
| Lipid-PEG (PEG- | (iv) DPG-PEG; | about 1.4% to about | about 1.4% to |
| lipid) | | 1.6% | about 1.6% |
| Targeting moiety | (v) a DSPE-PEG3.4K-anti-CD8α antibody conjugate, wherein the anti-CD8α antibody | about 0.01% to | about 0.01% to |
| | comprising SEQ ID NP: 9, SEQ ID NO: 169 or SEQ ID NO: 44. | 0.02% | 0.02% |

TABLE F-2
Exemplary LNP formulation (g/g mRNA)

| | | g/g mRNA | g/g mRNA |
|---|---|---|---|
| Components | Description | (Formulation 3) | (Formulation 4) |
| Ionizable cationic | (i) ionizable cationic Lipid 15 having the structure below, | about 14 to about | about 14 to |
| lipid | | 15 | about 15 |
| | 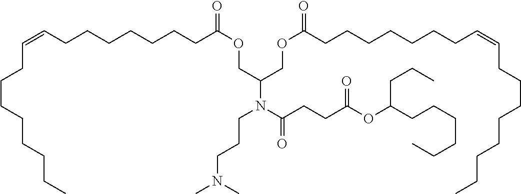 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC] | | |
| | Lipid 15 | | |
| | and/or a | | |
| | salt thereof; | | |
| Structural lipid | (ii) cholesterol; | about 4 to about 5 | about 3 to |
| | | | about 4 |
| Neutral lipid | (iii) DSPC; | about 2 to about 3 | about 4 to |
| | | | about 5 |
| Lipid-PEG (PEG- | (iv) DPG-PEG; | about 1.0 to about | about 1.0 to |
| lipid) | | 1.5 | about 1.5 |
| Targeting moiety | (v) a DSPE-PEG3.4K-anti-CD8α antibody conjugate, wherein the anti-CD8α | about 0.05 to about | about 0.05 to |
| | antibody comprising SEQ ID NP: 9, SEQ ID NO: 169 or SEQ ID NO: 44. | 0.10 | about 0.10 |

In some embodiments, an LNP as described in Table F-1 or Table F-2 herein comprises a payload mRNA comprising a nucleic acid sequence encoding a polypeptide comprising SEQ ID NO: 116, SEQ ID NO: 126, or SEQ ID NO: 127, wherein the mRNA comprises a 5′ cap (e.g., Cap 1), 5′ UTR, nucleic acids encoding a CD22 CAR, 3′ UTR, and a poly(A) tail. In some embodiments, an LNP as described in Table F-1 or Table F-2 herein comprises a payload mRNA comprising SEQ ID NO: 108, SEQ ID NO: 116, SEQ ID NO: 124, or SEQ ID NO: 126. In some embodiments, an LNP as described in Table F-1 or Table F-2 herein comprises a payload mRNA comprising SEQ ID NO: 139 or SEQ ID NO: 147.

### F. Additional Targeting Moieties

In some embodiments, the LNP further comprises one or more additional targeting moieties. In some embodiments, at least one additional targeting moiety is an anti-CD4 antibody (e.g., Ibalizumab). In some embodiments, the additional targeting moiety is in the same lipid-antibody conjugate for the anti-CD8 antibody, or in a different lipid-antibody conjugate. In some embodiments, the additional targeting moiety is in the same lipid-antibody conjugate for the anti-CD8 antibody, and the antibody in the conjugate is a bi-specific antibody targeting both CD8 and CD4. In some embodiments, the additional targeting moiety is in a different lipid-antibody conjugate. In some embodiments, the additional targeting moiety is an anti-CD4 antibody (e.g., Ibalizumab) in a different lipid-antibody conjugate. In some embodiments, the anti-CD8 conjugate and the anti-CD4 conjugate are presented in the LNP at a density of about 1.9 to 5.7 micromoles of conjugate per gram of mRNA in the LNP and about 1.9 to 11.4 micromoles of conjugate per gram of mRNA in the LNP respectively. In some embodiments, the anti-CD8 conjugate has a density of about 1.9 micromoles of conjugate per gram of mRNA in the LNP and the anti-CD4 conjugate has a density of about 11.4 micromoles of conjugate per gram of mRNA in the LNP.

### IV. Methods of Making LNPs

a. Method of Producing LNPs

In some embodiments, the LNPs are produced by using either rapid mixing by an orbital vortexer or by microfluidic mixing. In some embodiments, the LNPs are produced by using T-mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation.

In certain embodiments, the resulting LNP compositions comprise, for example, from about 40 mol % to about 60 mol % of one or more ionizable cationic lipids described herein, from about 35 mol % to about 50 mol % of one or more sterols, from about 5 mol % to about 15 mol % of one or more neutral lipids, and from about 0.5 mol % to about 5 mol % of one or more PEG-lipids.

In certain embodiments, the resulting LNP compositions comprise, for example, from about 40 mol % to about 60 mol % of one or more ionizable cationic lipids described herein, from about 25 mol % to about 40 mol % of one or more sterols, from about 15 mol % to about 25 mol % of one or more neutral lipids, and from about 0.5 mol % to about 5 mol % of one or more PEG-lipids.

b. Physical Properties of LNPs

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 70 nm to about 100 nm.

In some embodiments, LNPs comprising an ionizable cationic lipid described herein, prepared and characterized using methods described herein, show average diameter change after a freeze-thaw of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change after a freeze-thaw of less than 30%. In some embodiments, the freeze-thaw and diameter measurements are conducted with 10% sucrose in MES pH 6.5 buffer.

In some embodiments, LNPs comprising an ionizable cationic lipid described herein, prepared and characterized using methods described herein, show average diameter change upon targeting antibody insertion of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change upon targeting antibody insertion of less than 15%. In some embodiments, the diameter change upon targeting antibody insertion is measured in pH 6.5 MES using a 37° C. incubation for 4 hours.

In some embodiments, LNPs comprising an ionizable cationic lipid described herein, prepared and characterized using methods described herein, have average LNP diameter of less than 50, 60, 70, 80, 90, 100, 110, 120, 130, 140, 150, 160, 170, 180, 190, or 200 nm. In some embodiments, LNPs comprising an ionizable cationic lipid described herein, prepared and characterized using methods described herein, have average LNP diameter of less than 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

In some embodiments, the LNP compositions or LNPs comprising an ionizable cationic lipid described herein, prepared and characterized using methods described herein, have polydispersity of less than 0.4, 0.3, 0.25, 0.2, 0.15, 0.1, or 0.05. In some embodiments, LNPs comprising an ionizable cationic lipid described herein, prepared and characterized using methods described herein, have polydispersity of less than 0.25.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 mV to about +30 mV or about +10 mV to +30 mV or about +20 mV to about +30 mV at pH 5.5 or pH 5, and/or a zeta potential of about −30 mV to about +5 mV or about −20 mV to about +15 mV at pH 7.4.

In some embodiments, the LNP compositions or LNPs comprising an ionizable cationic lipid described herein, prepared and characterized using methods described herein, have Zeta Potential at pH 7.4 greater than −15, −14, −13, −12, −11, −10, −9, −8, −7, −6, −5.5, −5, −4.5, −4, −3.5, −3, −2.5, −2, −1.5, −1, or −0.5 mV. In some embodiments, the LNP compositions or LNPs comprising an ionizable cationic lipid described herein, prepared and characterized using methods described herein, have Zeta Potential at pH 7.4 greater than −10 mV. In some embodiments, the LNP compositions or LNPs comprising an ionizable cationic lipid described herein, prepared and characterized using methods described herein, have Zeta Potential at pH 7.4 greater than −1 mV. In some embodiments, the LNP compositions or LNPs comprising an ionizable cationic lipid described herein, prepared and characterized using methods described herein, have Zeta Potential at pH 5.5 greater than −1, 0, 1, 2, 3, 4, 4.5, 5, 7.5, 10, 12.5, 15, 17.5, 20, 22.5, or 25 mV. In some embodiments, the LNP compositions or LNPs comprising an ionizable cationic lipid described herein, prepared and characterized using methods described herein, have Zeta Potential at pH 5.5 greater than 5 mV. In some embodiments, the LNP compositions or LNPs comprising an ionizable cationic lipid described herein, prepared and characterized using methods described herein, have Zeta Potential at pH 5.5 greater than 15 mV.

### IV. Methods of Using LNPs

The present disclosure provides methods of delivering a payload to a target cell or tissue, for example, a target cell or tissue in a subject, and LNPs or pharmaceutical compositions containing the LNPs for use in such methods. Any disclosure herein of a method of, e.g., treating a disease or disorder or, e.g., delivering a nucleic acid to a cell or, e.g., producing a polypeptide of interest in a cell should be interpreted also as a disclosure of an LNP or pharmaceutical composition comprising said LNP for use in such methods.

In certain embodiments, the present disclosure provides a method of producing a polypeptide of interest (e.g., a protein of interest) in a mammalian cell, and LNPs or pharmaceutical compositions containing the LNPs for use in such methods. Methods of producing polypeptides in such a cell involve contacting a cell with an LNP composition comprising an RNA of interest (e.g., an mRNA encoding the polypeptide of interest (e.g., a protein of interest). Upon contacting the cell with the LNP composition, the mRNA may be taken up and translated in the cell to produce the polypeptide of interest.

In certain embodiments, the present disclosure provides methods for expressing a payload mRNA in a target cell, tissue or organ in a subject, and mRNAs, LNPs or pharmaceutical compositions containing the mRNAs or LNPs for use in such methods. In some embodiments, the subject is diagnosed to have, expected to have, or expected to develop a lymphoma. In some embodiments, the lymphoma is a B-cell lymphoma. In some embodiments, the B-cell lymphoma is diffuse large B-cell lymphoma (DLBCL). In some embodiments, the DLBCL is a relapse/refractory DLBCL. In some embodiments, the methods comprise contacting mRNAs, LNPs or pharmaceutical compositions containing the mRNA or LNPs described herein with a target cell, tissue, or organ of the subject.

In certain embodiments, the present disclosure provides methods for modifying gene expression in a target cell, issue or organ in a subject, and mRNAs, LNPs or pharmaceutical compositions containing the LNPs for use in such methods. In some embodiments, the methods comprise contacting a target cell, tissue, or organ in the subject with mRNAs, LNPs or pharmaceutical compositions containing the mRNA or LNPs described herein.

In certain embodiments, the present disclosure provides methods for modifying immune response in a subject, and mRNAs, LNPs or pharmaceutical compositions containing the mRNAs or LNPs for use in such methods. In some embodiments, the methods comprise administering the mRNAs, the LNPs or pharmaceutical compositions to the subject.

In certain embodiments, the present disclosure provides methods for activating T cells in a subject, and mRNAs, LNPs or pharmaceutical compositions containing the mRNAs or LNPs for use in such methods. In some embodiments, the methods comprise administering the mRNAs, the LNPs or the pharmaceutical compositions to the subject.

In certain embodiments, the present disclosure provides methods for inducing immune response against a cancerous cell in a subject, and mRNAs, LNPs or pharmaceutical compositions containing the mRNAs or LNPs for use in such methods. In some embodiments, the cancerous cell is a lymphoma cell. In some embodiments, the lymphoma cell is a B cell. In some embodiments, the methods comprise administering the mRNAs, the LNPs or the pharmaceutical compositions to the subject.

In certain embodiments, the present disclosure provides methods for treating B-cell lymphoma in a subject, and mRNAs, LNPs or pharmaceutical compositions containing the mRNAs or LNPs for use in such methods. In some embodiments, the B-cell lymphoma is diffuse large B-cell lymphoma (DLBCL). In some embodiments, the DLBCL is a relapse/refractory DLBCL. In some embodiments, the methods comprise administering the mRNAs, the LNPs or the pharmaceutical composition to the subject.

In certain embodiments, the present disclosure provides methods for producing a modified T-cell in vitro, ex vivo, or in vivo, and mRNAs, LNPs or pharmaceutical compositions containing the mRNAs or LNPs for use in such methods. In some embodiments, the methods comprise contacting the mRNAs, the LNPs or the pharmaceutical compositions to a T-cell.

In certain embodiments, the present disclosure provides methods for producing a T-cell with a Chimeric Antigen Receptor (CAR), and mRNAs, LNPs or pharmaceutical compositions containing the mRNAs or LNPs for use in such methods. In some embodiments, the methods comprise contacting the mRNAs, the LNPs or pharmaceutical compositions to a T-cell.

In certain embodiments, the present disclosure provides methods for down-regulating B-cell lymphoma activity in a subject, and mRNAs, LNPs or pharmaceutical compositions containing the mRNAs or LNPs for use in such methods. In some embodiments, the B-cell lymphoma is diffuse large B-cell lymphoma (DLBCL). In some embodiments, the DLBCL is a relapse/refractory DLBCL. In some embodiments, the methods comprise administering the mRNAs, the LNPs or the pharmaceutical compositions to the subject.

In certain embodiments, the present disclosure provides methods, wherein the methods comprise contacting a cell, tissue, or organ in a subject with mRNAs, LNPs or pharmaceutical compositions containing the mRNAs or LNPs as described herein.

In certain embodiments, the present disclosure provides methods, wherein the methods comprise administering a subject with mRNAs, LNPs or pharmaceutical compositions containing the mRNAs or LNPs as described herein.

In general, the step of contacting a mammalian cell with an mRNA or an LNP composition including an mRNA encoding a polypeptide of interest may be performed in vivo, ex vivo, or in vitro. The amount of the mRNA or the LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the mRNA or the LNP composition will allow for efficient polypeptide production in the cell. Metrics for efficiency may include polypeptide translation (indicated by polypeptide expression), level of mRNA degradation, and immune response indicators.

The step of contacting an mRNA or an LNP composition including an mRNA of the present disclosure with a cell may involve or cause transfection where the LNP composition or other vehicle for delivery the mRNA may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the present disclosure may encode a polypeptide that may improve or increase the immunity of a subject.

In certain embodiments, contacting a cell with an mRNA or an LNP composition including an mRNA of the present disclosure may reduce the innate immune response of a cell to an exogenous nucleic acid. A cell may be contacted with a first exogenous mRNA or a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T-cell. Upon contacting the cell with the LNP composition, a translatable mRNA may be translated in the cell to produce a polypeptide of interest.

In certain embodiments, an LNP composition of the present disclosure may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the immune cell targeting group of the LNPs) as compared to another destinations (e.g., cells that either do not express or only express at low levels the receptor of interest).

mRNAs or LNP compositions comprising mRNAs of the present disclosure may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the present disclosure may be useful in the treatment of acute diseases, disorders, or conditions such as sepsis, stroke, and myocardial infarction. An mRNA included in an LNP composition of the present disclosure may also be capable of altering the rate of transcription of a given species, thereby affecting gene expression.

Diseases, disorders, and/or conditions characterized by dysfunctional or aberrant protein or polypeptide activity for which a composition of the present disclosure may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. The present disclosure provides a method for treating such diseases, disorders, and/or conditions in a subject by administering a composition including an mRNA as described herein (e.g., an LNP composition comprising the mRNA) to the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

An mRNA or a LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an mRNA composition (e.g., an LNP composition comprising the mRNA) may be administered intravenously, intramuscularly, intradermally, intra-arterially, intratumorally, or subcutaneously. However, the present disclosure encompasses the delivery of mRNA (e.g., LNP compositions comprising the mRNA) of the present disclosure by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

mRNAs or LNP compositions including one or more mRNAs of the present disclosure may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the present disclosure, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the immune cell or a polypeptide encoded by the nucleic acid delivered by the LNP and expressed in the immune cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the immune cells or a polypeptide encoded by the nucleic acid delivered by the reference LNP and expressed in the immune cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of immune cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-KC3-DMA, but otherwise as the same as a tested LNP. In some embodiments, the reference LNP comprises cationic Lipid DLin-KC2-DMA, but otherwise as the same as a tested LNP. In some embodiments, the reference LNP comprises cationic Lipid ALC-0315, but otherwise as the same as a tested LNP. In some embodiments, the reference LNP comprises cationic Lipid SM-102, but otherwise as the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the immune cells are transfected by the LNP. In some embodiments, the immune cells are subject's immune cells. In some embodiments, the immune cells are immune cells targeted by the method. In some embodiments, the immune cells are subject's immune cells targeted by the method.

In some embodiments, the expression level of the nucleic acid delivered by the LNP is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition containing thereof, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP). In some embodiments, the LNP is an LNP as described herein in the present disclosure.

In some embodiments, the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]
[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition containing thereof, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore, to treat or ameliorate the symptom. In some embodiments, an aspect of the disclosure relates to an LNP or a pharmaceutical composition containing thereof, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vivo, in vitro, or ex vivo the immune cell of a subject with a lipid nanoparticle (LNP).

In some embodiments, the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP;
and
(vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen. In some embodiment, the disorder is lymphoma. In some embodiments, the lymphoma is a B-cell lymphoma. In some embodiments, the B-cell lymphoma is diffuse large B-cell lymphoma (DLBCL). In some embodiments, the DLBCL is a relapse/refractory DLBCL.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspects, provided are methods of targeting the delivery of a nucleic acid to an immune cell of a subject. In some embodiments, the method comprises contacting the immune cell with a lipid nanoparticle (LNP) provided herein. In some embodiments, the method is for targeting T cells. In some embodiments, the immune cell targeting group binds to CD8α.

In some aspects, provided are methods of expressing a polypeptide of interest in a targeted immune cell of a subject. In some embodiments, the method comprises contacting the immune cell with an mRNA or a lipid nanoparticle (LNP) provided herein.

In some aspect, provided are method of modulating cellular function of a target immune cell of a subject. In some embodiments, the method comprises administering to the subject an mRNA or a lipid nanoparticle (LNP) provided herein.

In some aspects, provided are method of treating, ameliorating, or preventing a symptom of a disorder or disease in a subject in need thereof. In some embodiments, the subject is a subject in need thereof. In some embodiments, the method comprises administering to the subject an mRNA or a lipid nanoparticle (LNP) provided herein.

In some aspects, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

In some embodiments, when a substance described herein such as the LNP, isolated polynucleotide, expression construct, vector, host cell, ISVD, polypeptide, construct, and/or composition described herein is administered to a subject, it is administered in an effective amount, therapeutically effective amount, and/or prophylactically effective amount. In some embodiments, when a substance such as the LNP, isolated polynucleotide, expression construct, vector, host cell, ISVD, polypeptide, construct, and/or composition described herein is administered to a subject, it is administered in a pharmaceutically active amount.

### Formulation and Mode of Delivery

LNP compositions of the present disclosure may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the present disclosure, except insofar as any conventional excipient or accessory ingredient may be incompatible with one or more components of an LNP composition of the present disclosure. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the present disclosure. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the present disclosure may be prepared in a variety of forms suitable for a variety of routes and methods of administration. For example, pharmaceutical compositions of the present disclosure may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### V. Kits

Another aspect of the present disclosure provides a kit. In some embodiments, the kits are for treating a disorder. In some embodiments, the kit comprises an mRNA as described herein. In some embodiments, the kit comprises an LNP as described herein. In some embodiments, the kit comprises: an ionizable cationic lipid, a lipid-immune cell targeting group conjugate, a lipid nanoparticle composition comprising an ionizable cationic lipid and/or a lipid-immune cell targeting group conjugate with or without an encapsulated payload (e.g., an mRNA), and instructions for treating a medical disorder, such as, cancer or a microbial or viral infection.

### EXAMPLES

The invention now being generally described, will be more readily understood by reference to the following examples, which are included merely for purposes of illustration of certain aspects and embodiments of the present invention, and are not intended to limit the invention.

### Example 1. Preparation of Ionizable Cationic Lipids

This Example describes the synthesis of various cationic lipids.

### General Scheme for the Synthesis of Lipids 1 Through Lipid 30

A general scheme for the synthesis of Lipids 1 through Lipid 30 is provided in Scheme 1 below. Corresponding R and R′ for each lipid is provided in Tables 2 to 4 below.

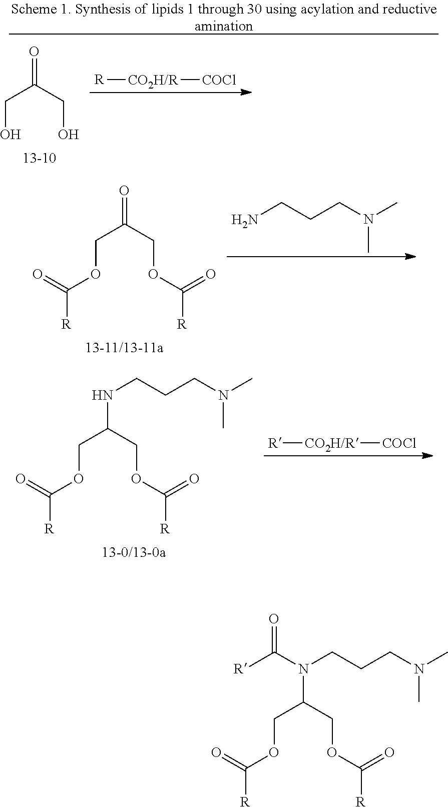

### Synthesis of Intermediates 13-11 and 13-11a

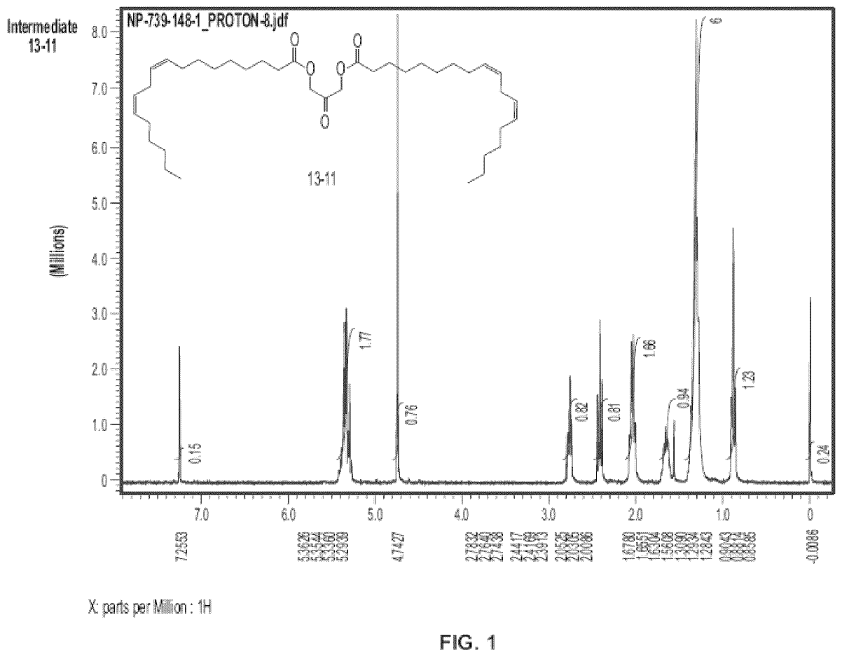

Intermediate 13-11 (Scheme 2) was synthesized by acylation of dihydroxyacetone (13-10) with linoleic acid. Dihydroxyacetone (22 mmol, 2 g, 1 eq.) was reacted with linoleic acid 1-5 (55 mmol, 15.4 g, 2.5 eq.) using EDCI (55 mmol, 10.5 g, 2.5 eq.) activation in 50 mL DCM, in the presence of DIPEA (55 mmol, 9.6 mL, 2.5 eq.), DMAP (4.4 mmol, 540 mg, 0.2 eq.) at room temperature yielding 11.1 g (79%) crude product. Purified product was obtained by column chromatography and characterized by proton NMR spectroscopy (FIG. 1).

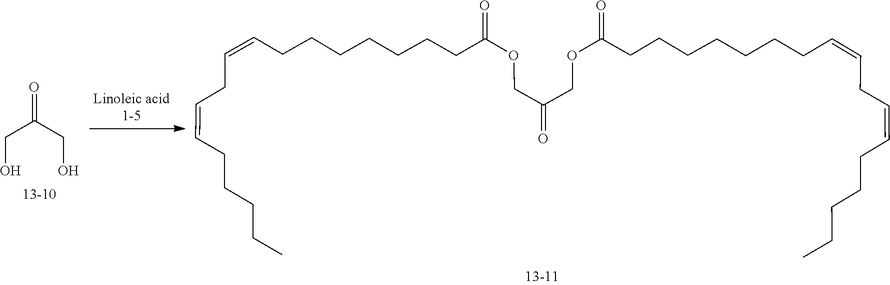 [SMILES: O=C(CO)CO ; CCCCC/C=C\C/C=C\CCCCCCCC(=O)OCC(=O)COC(=O)CCCCCCC/C=C\C/C=C\CCCCC]

Intermediates 13-11a (Scheme 3) was synthesized by acylation of dihydroxyacetone (13-10) with oleoyl chloride. Dihydroxyacetone (44.4 mmol, 4 g, 1 eq.) was reacted with oleoyl chloride 1-6a (111 mmol, 36.7 mL, 2.5 eq.) in the presence of Pyridine (133.3 mmol, 11 mL, 3 eq.), DMAP (13.3 mmol, 1.63 g, 0.3 eq.) in 80 mL DCM, at room temperature yielding 14.9 g (54%) crude product. Crude product was purified by column chromatography and characterized by proton NMR spectroscopy (FIG. 2A).

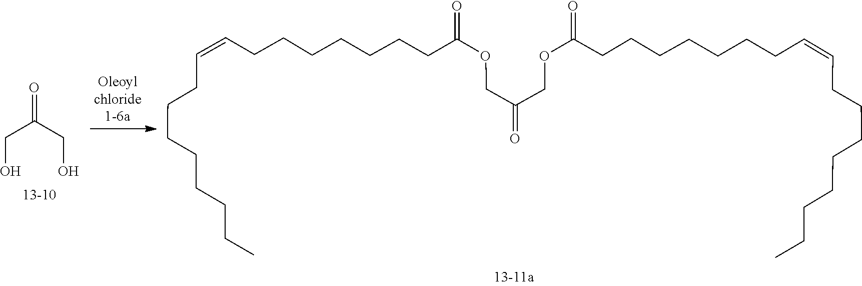 [SMILES: O=C(CO)CO ; CCCCCCCC/C=C\CCCCCCCC(=O)OCC(=O)COC(=O)CCCCCCC/C=C\CCCCCCCC]

### Synthesis of Intermediates 13-0a and 13-11b

Intermediates 13-0a and 13-11b were synthesized by reductive amination of intermediates 13-11 and 13-11a respectively.

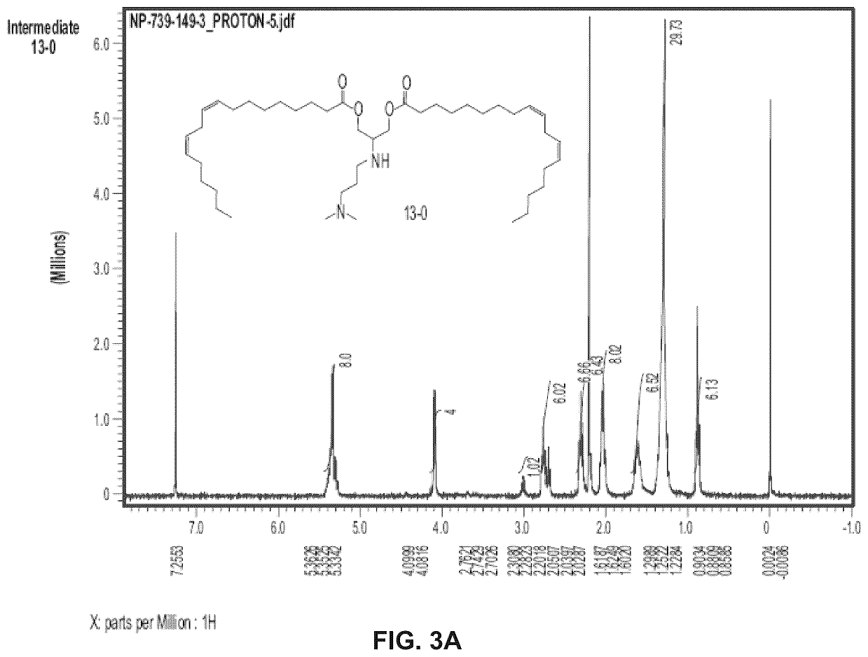

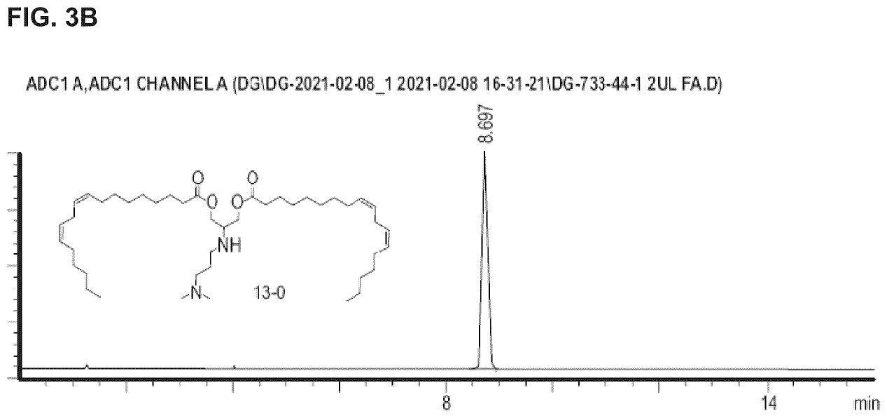

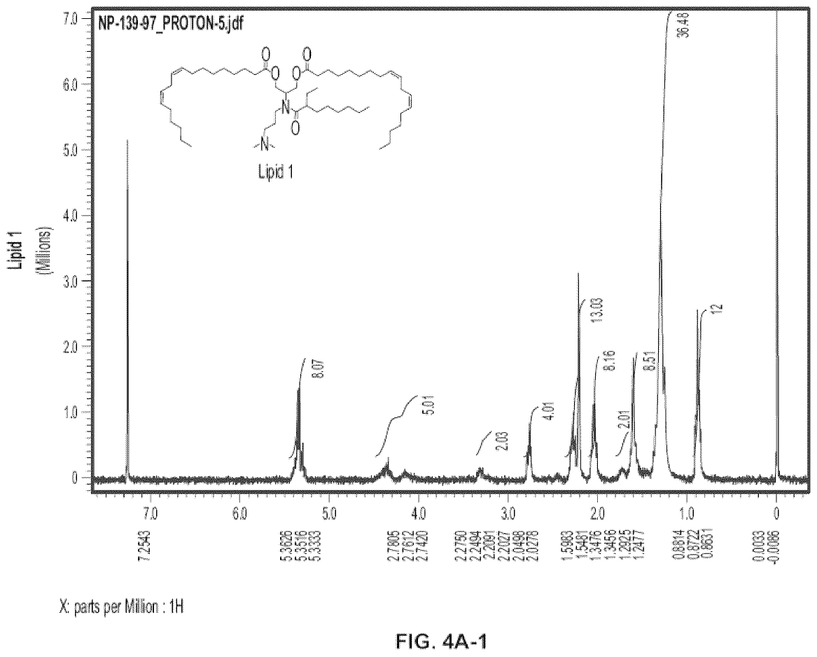

Intermediate 13-0 was produced by reductive amination (scheme 4) of intermediate 13-11 (13.1 mmol, 8.1 g, 1.0 eq) using N1,N1-dimethylpropane-1,3-diamine 15-3 (26 mmol, 3.2 mL, 2.0 eq.) in DCM (10 mL) using acetic acid (26.0 mmol, 1.50 mL, 2 eq.) and sodium borohydride triacetate (4.32 mmol, 3.3 g, 1.2 eq.) yielding 3.1 g (32%) crude product. Column purification resulted in pure product (Proton NMR Spectrum and LC-CAD chromatogram shown in FIG. 3A and FIG. 3B, respectively).

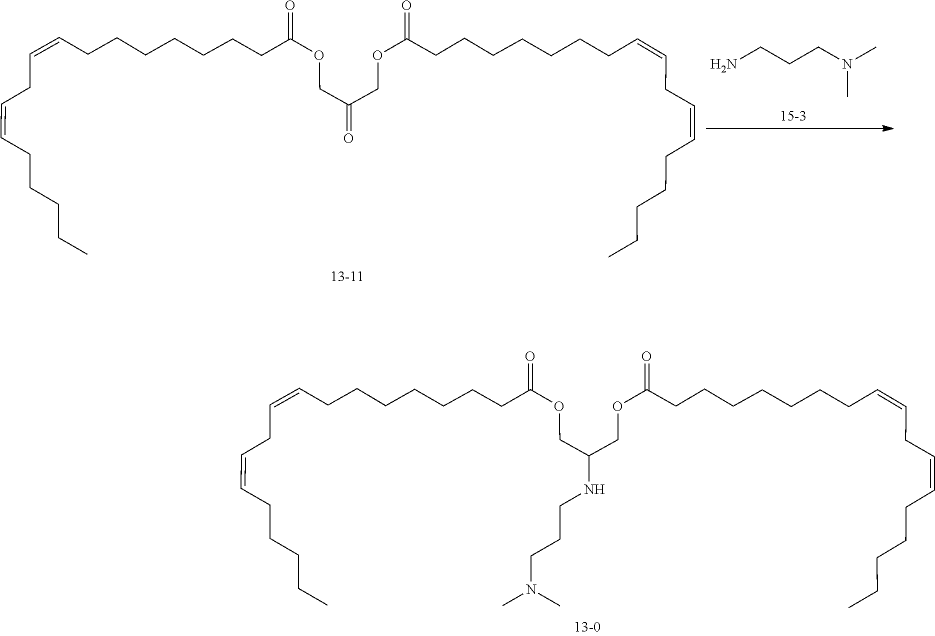

Intermediate 13-11b was produced by reductive amination (scheme 5) of intermediate 13-11a (24.2 mmol, 14.9 g, 1.0 eq) using N1,N1-dimethylpropane-1,3-diamine 15-3 (48.4 mmol, 6.05 mL, 2.0 eq.) in DCM (60 mL) using acetic acid (48.4 mmol, 2.8 mL, 2 eq.) and sodium borohydride triacetate (29.1 mmol, 6.05 g, 1.2 eq.) yielding 6 g (35%) crude product. Column purification resulted in purified product (Proton NMR Spectrum and LC-ELSD chromatogram shown in FIG. 2B and FIG. 2C, respectively).

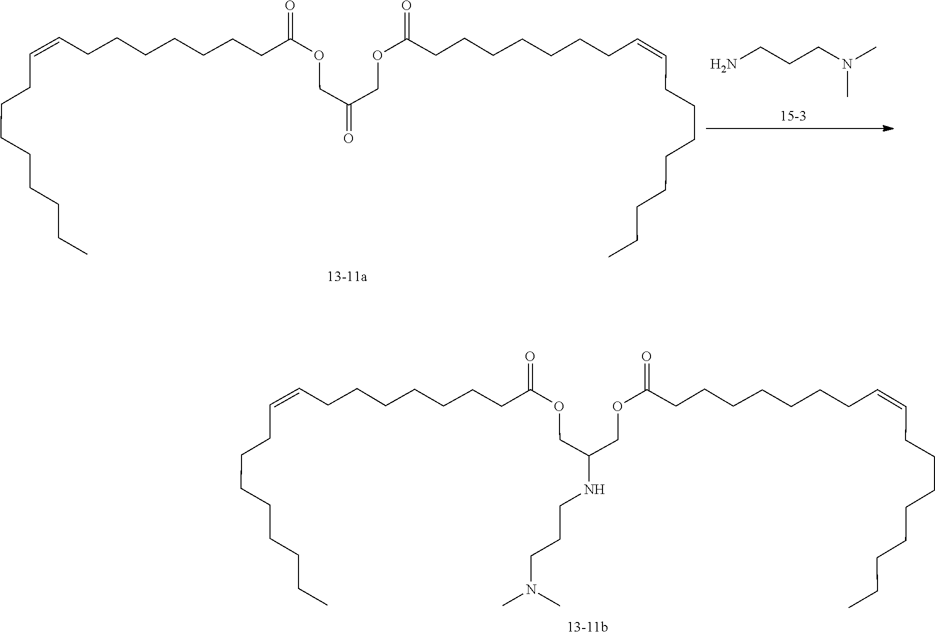

TABLE 2
R (O-acyl) and R′ (N-acyl) groups of lipids 1 through 8

| Lipid # | R (O-acyl groups) | R′ (N-acyl group) |
|---|---|---|
| Lipid 1 | 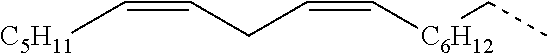 | 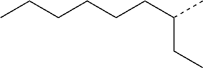 |
| Lipid 2 | 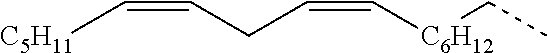 | 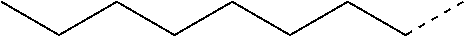 |
| Lipid 3 | 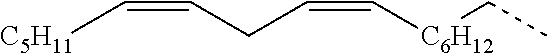 | 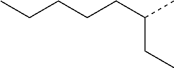 |
| Lipid 4 | 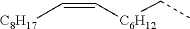 | 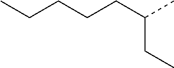 |
| Lipid 5 | 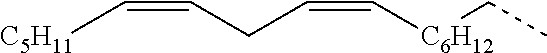 | 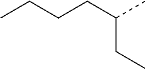 |
| Lipid 6 | 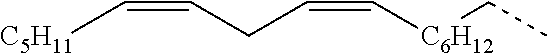 | 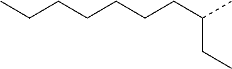 |
| Lipid 7 | 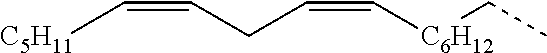 | 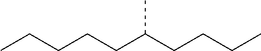 |
| Lipid 8 | 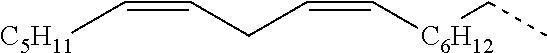 | 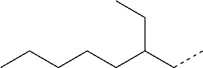 |

TABLE 3
R (O-acyl) and R′ (N-acyl) groups of Lipids 9 through 16

| Lipid # | R (O acyl groups) | R′ (N-acyl group) |
|---|---|---|
| Lipid 9 | 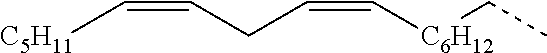 | 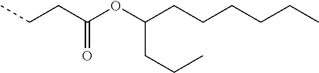 |
| Lipid 10 | 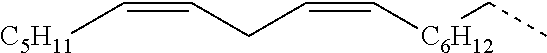 | 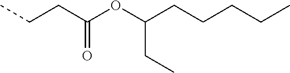 |
| Lipid 10A | 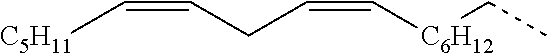 | 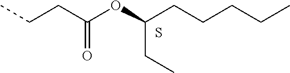 |
| Lipid 11 | 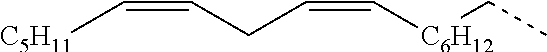 | 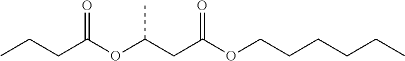 [SMILES: CCCCCCOC(=O)C[C@@H](C)OC(=O)CCC] |
| Lipid 11A | 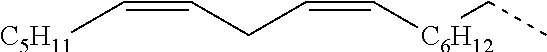 | 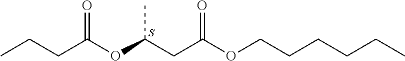 |
| Lipid 12 | 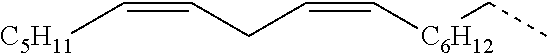 | 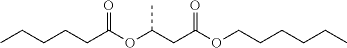 [SMILES: CCCCCCOC(=O)C[C@@H](C)OC(=O)CCCCC] |
| Lipid 13 | 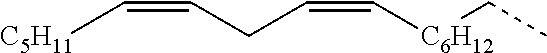 | 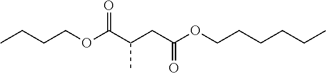 [SMILES: CCCCCCOC(=O)C[C@H](C)C(=O)OCCCC] |
| Lipid 14 | 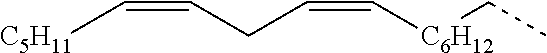 | 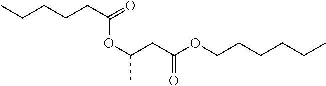 [SMILES: CCCCCCOC(=O)C[C@H](C)OC(=O)CCCCC] |
| Lipid 14A | 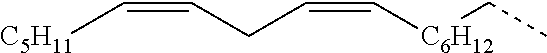 | 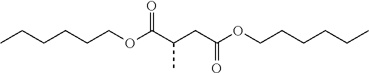 [SMILES: CCCCCCOC(=O)C[C@H](C)C(=O)OCCCCCC] |
| Lipid 15 | 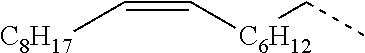 | 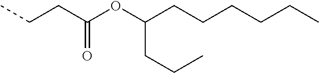 |
| Lipid 16 | 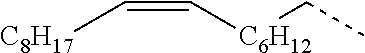 | 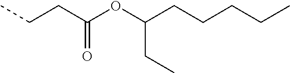 |

TABLE 4-1
R (O-acyl) and R′ (N-acyl) groups of Lipids 17, 17A, 18, 18A, 19, 19A, 20, 20A, 21, 21A, 22, 23, and 23A

| Lipid # | R (O-acyl groups) | R′ (N-acyl group) |
|---|---|---|
| Lipid 17 | 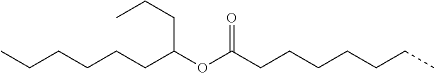 | 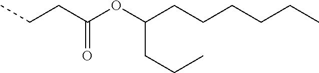 |
| Lipid 17A | 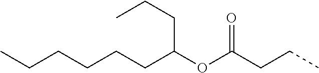 | 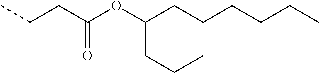 |
| Lipid 18 | 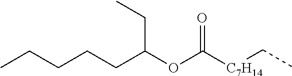 | 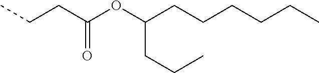 |
| Lipid 18A | 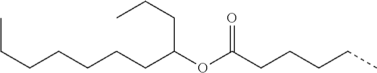 | 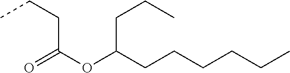 |
| Lipid 19 | 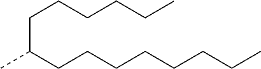 | 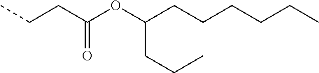 |
| Lipid 19A | 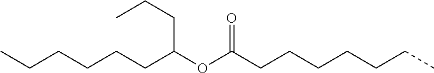 | 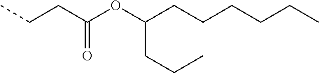 |
| Lipid 20 | 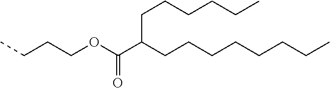 | 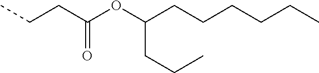 |
| Lipid 20A | 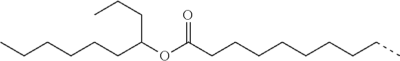 | 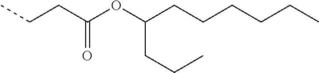 |
| Lipid 21 | 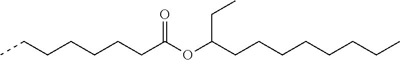 | 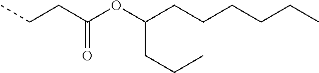 |
| Lipid 21A | 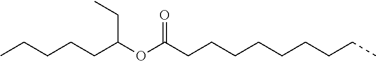 | 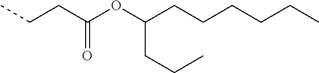 |
| Lipid 22 | 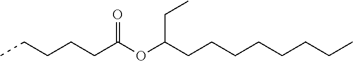 | 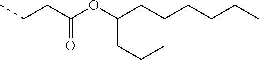 |
| Lipid 23 | 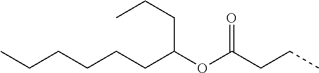 | 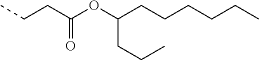 |
| Lipid 23A | 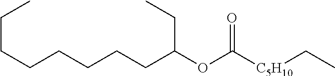 | 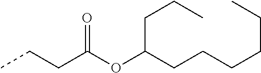 |

TABLE 4-2
R (O-acyl) and R′ (N-acyl) groups of Lipids 24, 25, 25A, 26, 27, 28, 29, and 30

| Lipid # | R (O-acyl groups) | R′ (N acyl group) |
|---|---|---|
| Lipid 24 | 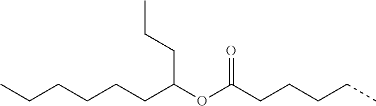 | 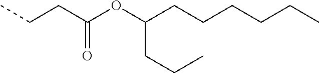 |
| Lipid 25 | 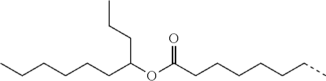 | 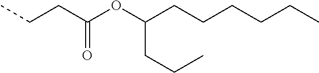 |
| Lipid 25A | 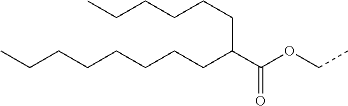 | 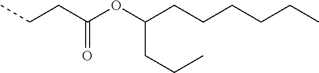 |
| Lipid 26 | 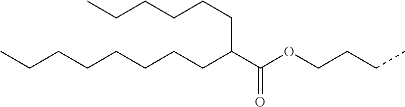 | 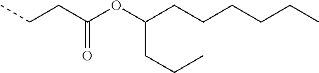 |
| Lipid 27 | 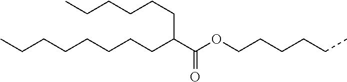 | 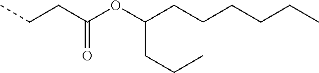 |
| Lipid 28 | 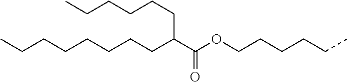 | 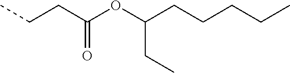 |
| Lipid 29 | 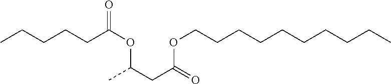 [SMILES: CCCCCCCCCCOC(=O)C[C@H](C)OC(=O)CCCCC] | 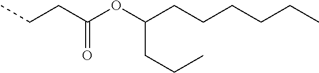 |
| Lipid 30 | 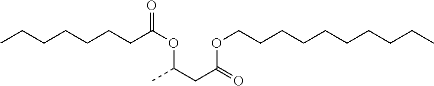 [SMILES: CCCCCCCCCCOC(=O)C[C@H](C)OC(=O)CCCCCCC] | 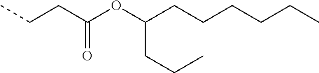 |

TABLE 4-3
R (O-acyl) and R′ (N-acyl) groups of Lipids 31 to 38, 37A, and 38A

| Lipid # | R (O-acyl groups) | R′ (N-acyl group) |
|---|---|---|
| Lipid 31 | 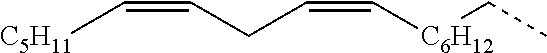 | 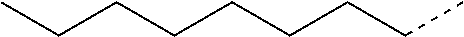 |
| Lipid 32 | 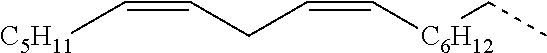 | 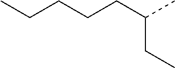 |
| Lipid 33 | 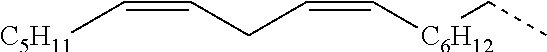 | 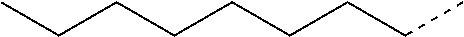 |
| Lipid 34 | 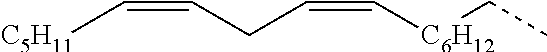 | 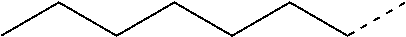 |
| Lipid 35 | 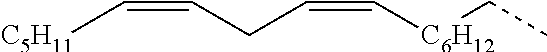 | 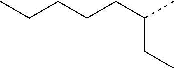 |
| Lipid 36 | 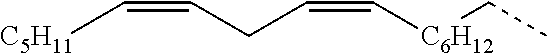 | 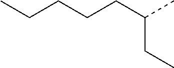 |
| Lipid 37 | 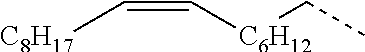 | 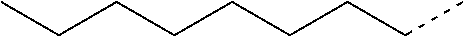 |
| Lipid 38 | 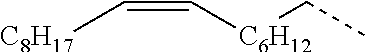 | 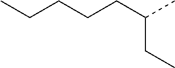 |
| Lipid 37A | 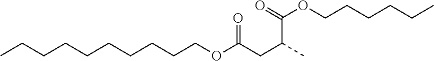 [SMILES: CCCCCCCCCCOC(=O)C[C@@H](C)C(=O)OCCCCCC] | 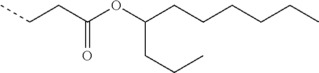 |
| Lipid 38A | 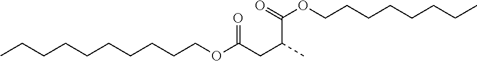 [SMILES: CCCCCCCCCCOC(=O)C[C@@H](C)C(=O)OCCCCCCCC] | 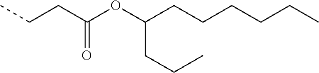 |

TABLE 5
Expected and observed mass (m/z) of named ionizable lipids

| | | Expected | |
|---|---|---|---|
| | | mass | |
| Entry | Compound code | (g/mol) | Observed mass (m/z) |
| 1 | Lipid 1 | 854.75 | 855.7, 856.7, 857.7 (M + 1, M + 2, M + 3) |
| 2 | Lipid 2 | 840.73 | 841.7, 842.7, 843.7 (M + 1, M + 2, M + 3) |
| 3 | Lipid 3 | 840.73 | 841.7, 842.7, 843.7 (M + 1, M + 2, M + 3) |
| 4 | Lipid 4 | 845.39 | 845.7, 846.7, 847.7 (M, M + 1, M + 2) |
| 5 | Lipid 5 (S) isomer | 827.33 | 827.7, 828.7, 829.7 (M, M + 1, M + 2) |
| | (Lipid 5A) | | |
| 6 | Lipid 6 | 868.76 | 869.7, 870.7, 871.7 (M + 1, M + 2, M + 3) |
| 7 | Lipid 7 | 868.76 | 869.7, 870.7, 871.7 (M + 1, M + 2, M + 3) |
| 8 | Lipid 8 | 854.75 | 855.7, 856.7, 857.7 (M + 1, M + 2, M + 3) |
| 9 | Lipid 9 | 940.78 | 941.7, 942.7. 943.7 (M + 1, M + 2, M + 3) |
| 10 | Lipid 10 (S) isomer | 912.75 | 913.7. 914.7. 915.7 (M + 1, M + 2, M + 3) |
| | (Lipid 10A) | | |
| 11 | Lipid 11 (S) isomer | 970.76 | 971.7, 972.7, 973.7 (M + 1, M + 2, M + 3) |
| | (Lipid 11A) | | |
| 12 | Lipid 12 | 999.51 | 999.0, 1001, 1002 (M + 1, M + 2, M + 3) |
| 13 | Lipid 13 | 984.77 | 985.7, 986.7, 987.6 (M + 1, M + 2, M + 3) |
| 14 | Lipid 14A | 1013.54 | 1013.1, 1014.1, 1015.1 (M + 1, M + 2, M + 3) |
| 15 | Lipid 15 | 944.82 | 945.1, 946.1, 947.1 (M + 1, M + 2, M + 3) |
| 16 | Lipid 16 | 916.78 | 917.2. 918.2, 919.2 (M + 1, M + 2, M + 3) |
| 17 | Lipid 17A | 896.67 | 897.9, 898.9, 899.9 (M + 1, M + 2, M + 3) |
| 18 | Lipid 18A | 952.73 | 953.7, 954.7, 955.7 (M + 1, M + 2, M + 3) |
| 19 | Lipid 19A | 1008.80 | 1009.8, 1010.8, 1011.8 (M + 1, M + 2, M + 3) |
| 20 | Lipid 20A | 1064.9 | 1065.7, 1066.7, 1067.7 (M + 1, M + 2, M + 3) |
| 21 | Lipid 21A | 1008.80 | 1009.7, 1010.7, 1011.7 (M + 1, M + 2, M + 3) |
| 22 | Lipid 22 | 980.76 | 981.7, 982.7, 983.7 (M + 1, M + 2, M + 3) |
| 23 | Lipid 23A | 1036.8 | 1037.7, 1038.6, 1039.7 (M + 1, M + 2, M + 3) |
| 24 | Lipid 19 (Lipid 24A) | 892.78 | 893.7, 894.7, 895.7 (M + 1, M + 2, M + 3) |
| 25 | Lipid 25A | 1008.80 | 1009.7, 1010.7, 1011.7 (M + 1, M + 2, M + 3) |
| 26 | Lipid 20 (Lipid 26) | 1064.86 | 1065.1, 1066.1, 1067.1, 1068.1 (M + 1, M + 2, M + 3, |
| | | | M + 4) |
| 27 | Lipid 27 | 1120.92 | 1121.9, 1122.9, 1123.9 (M + 1, M + 2, M + 3) |
| 28 | Lipid 28 | 1092.9 | 1093.8, 1094.8, 1095.8 (M + 1, M + 2, M + 3) |
| 29 | Lipid 29 | 1124.81 | 1125.8, 1126.8, 1127.8 (M + 1, M + 2, M + 3) |
| 30 | Lipid 30 | 1180.87 | NA |
| 31 | Lipid 31 | 854.75 | 855.1, 856.1, 857.1 (M + 1, M + 2, M + 3) |
| 32 | Lipid 32 | 854.75 | 855.7, 856.7, 857.7 (M + 1, M + 2, M + 3) |
| 33 | Lipid 33 | 840.73 | 841.7, 842.7, 843.7 (M + 1, M + 2, M + 3) |
| 34 | Lipid 34 | 868.76 | 869.7. 870.7. 871.7 (M + 1, M + 2, M + 3) |
| 35 | Lipid 35 | 914.77 | NA |
| 36 | Lipid 36 | 884.76 | NA |
| 37 | Lipid 37A | 1153.63 | 1153.8, 1154.8, 1155.8 (M + 1, M + 2, M + 3) |
| 38 | Lipid 38A | 1209.74 | NA |

### Synthesis of Lipids 1-16 by N-Acylation of Intermediates 13-0 or 13-11b

N-acylation of intermediates 13-0 and 13-11b with compounds R′CO₂H or R′COCl (R′ structures shown in Table 2 and Table 3) yielded lipids 1 through 16 as described in examples below.

### Synthesis of Lipids 1, 3, 4, 5, 6, and 7 by N-Acylation of Intermediate 13-0 Using the Corresponding Acid Chlorides

### Synthesis of Lipid 1

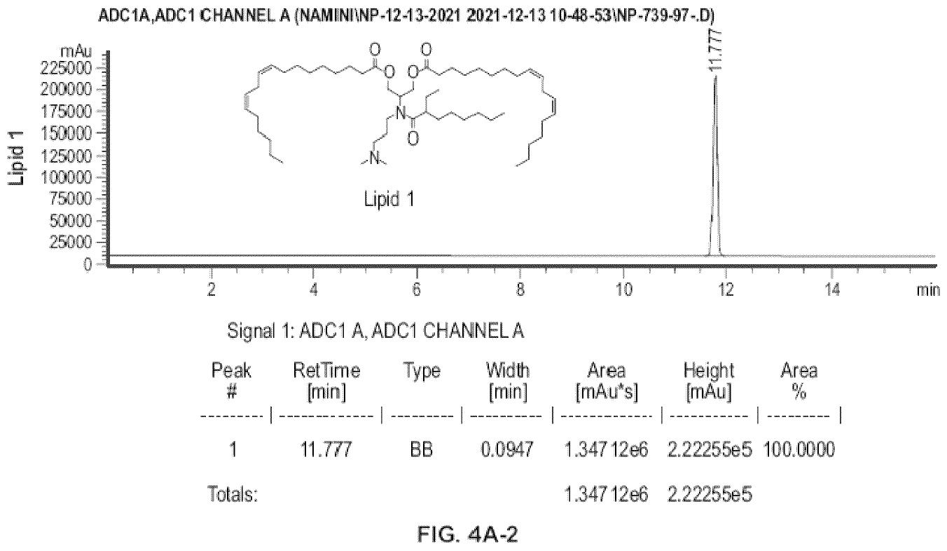

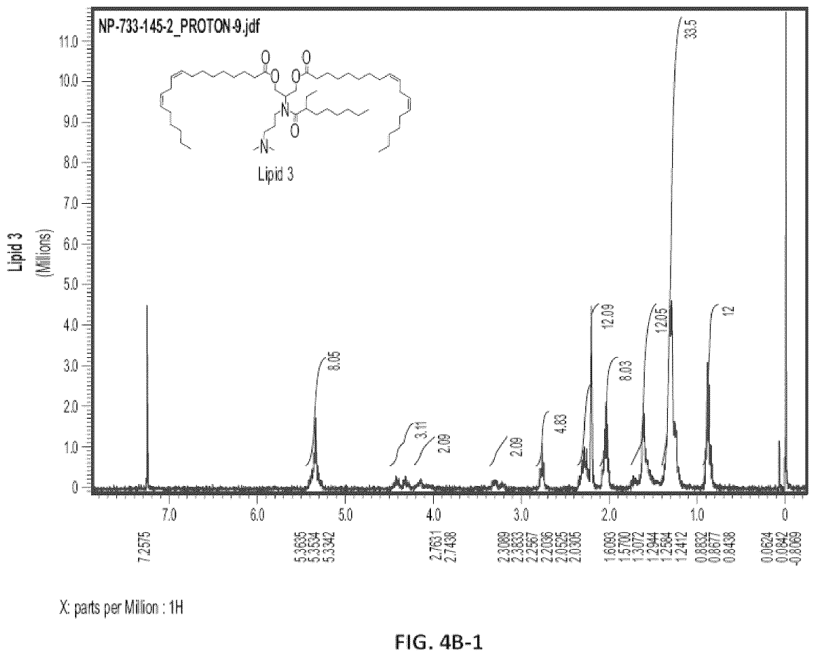

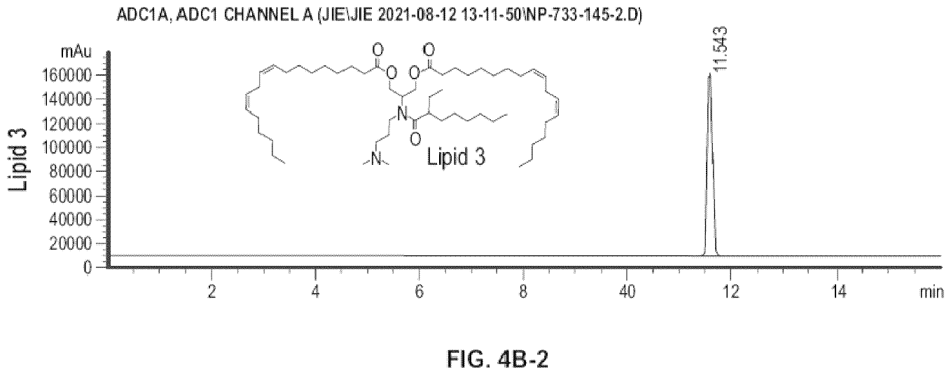

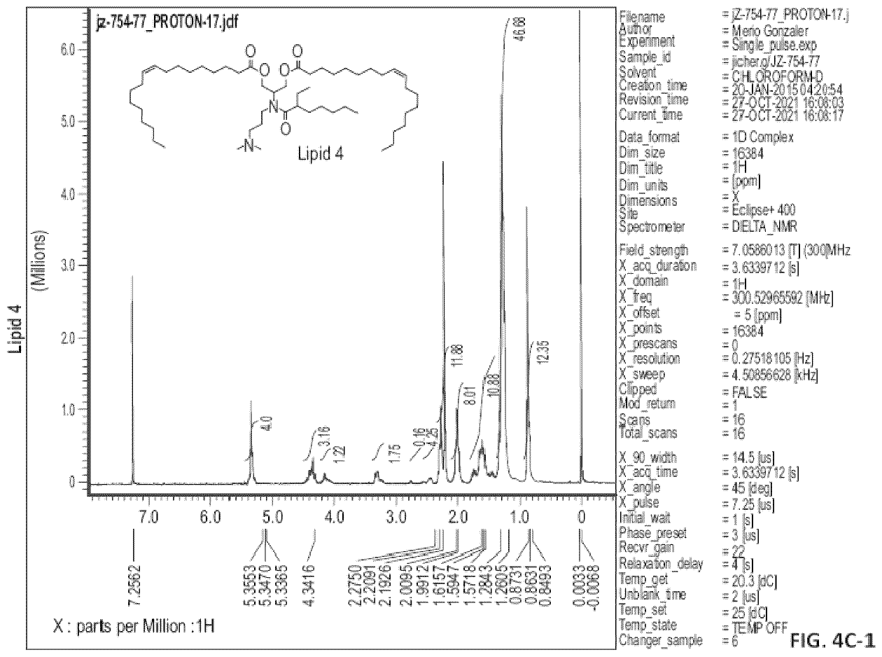

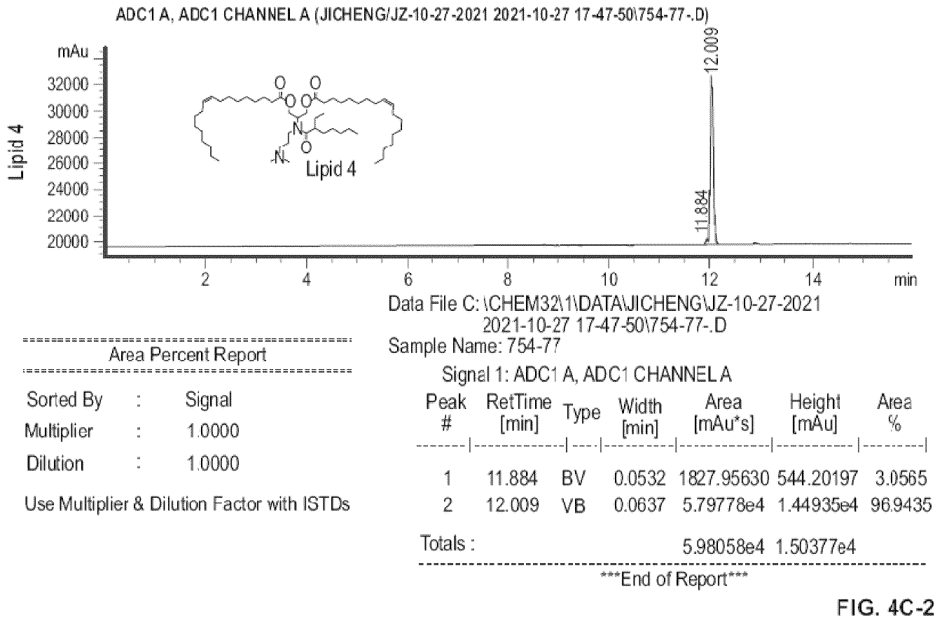

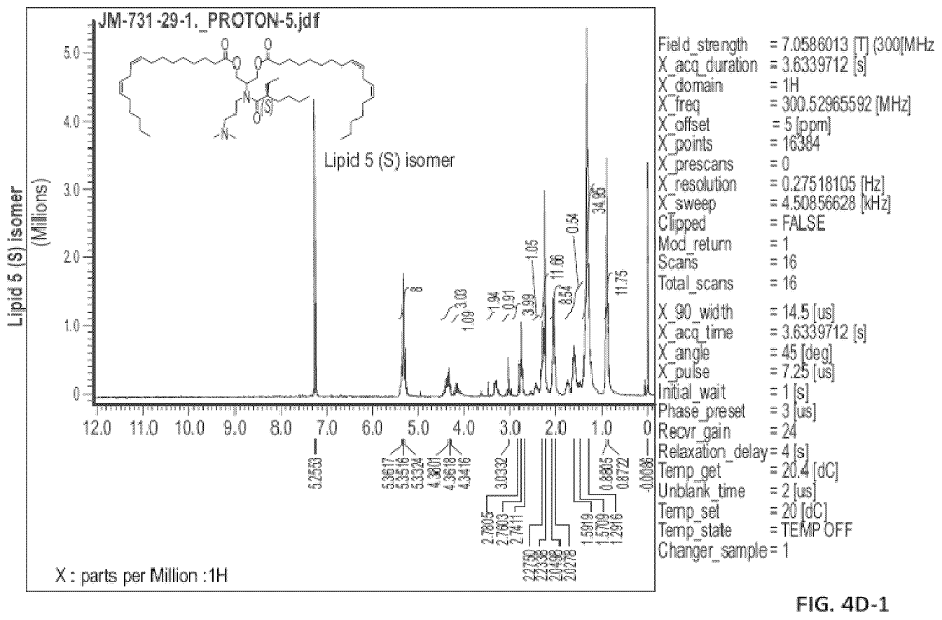

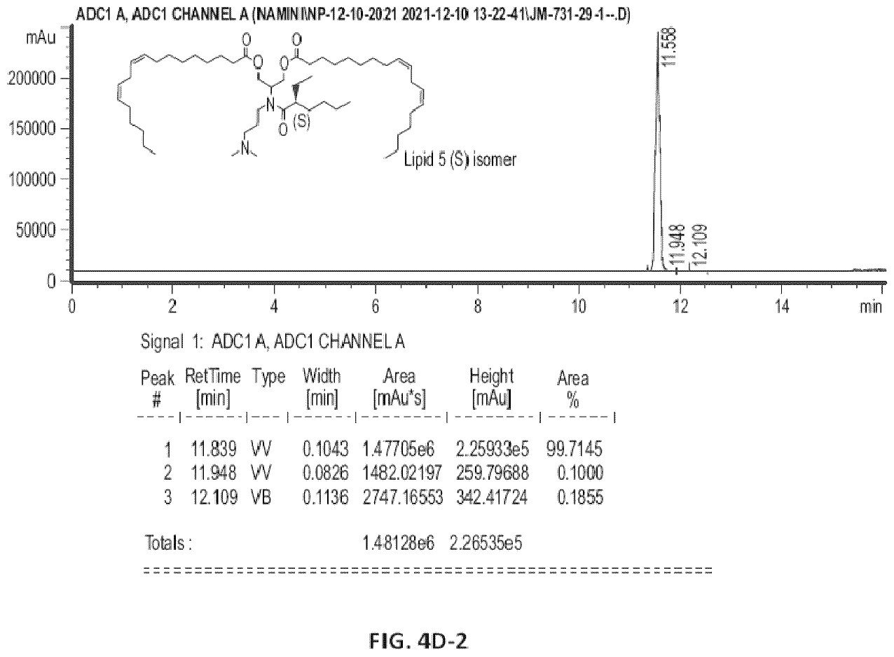

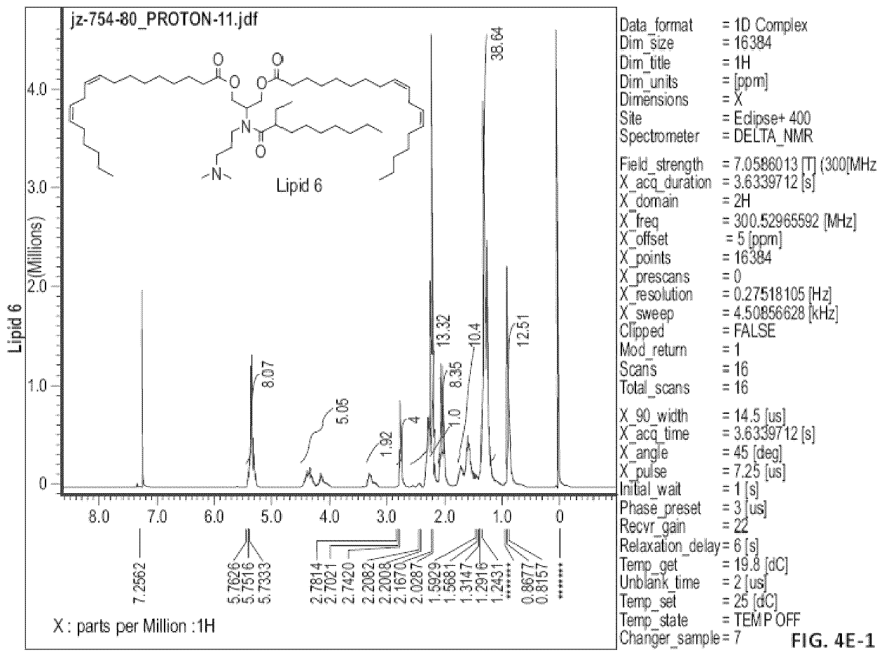

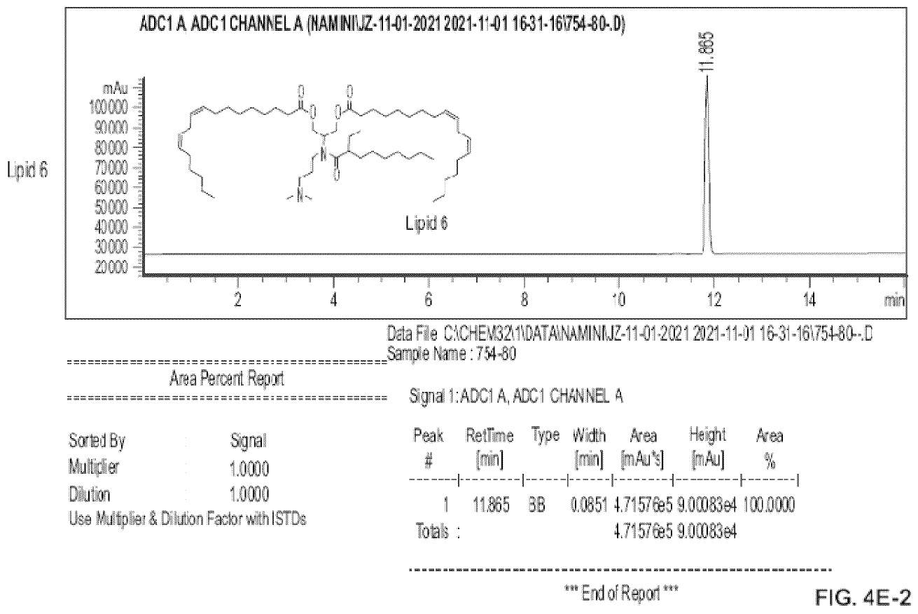

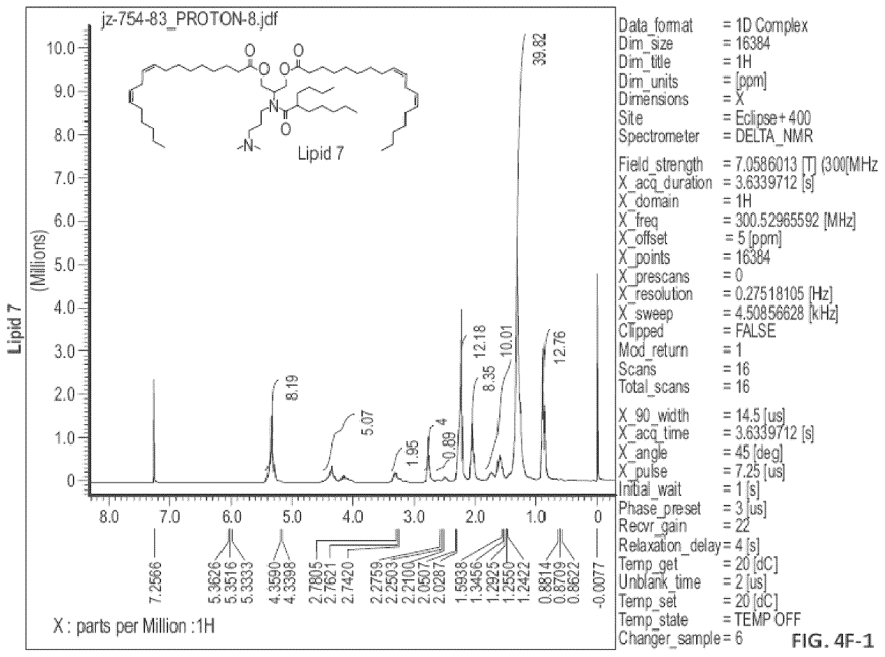

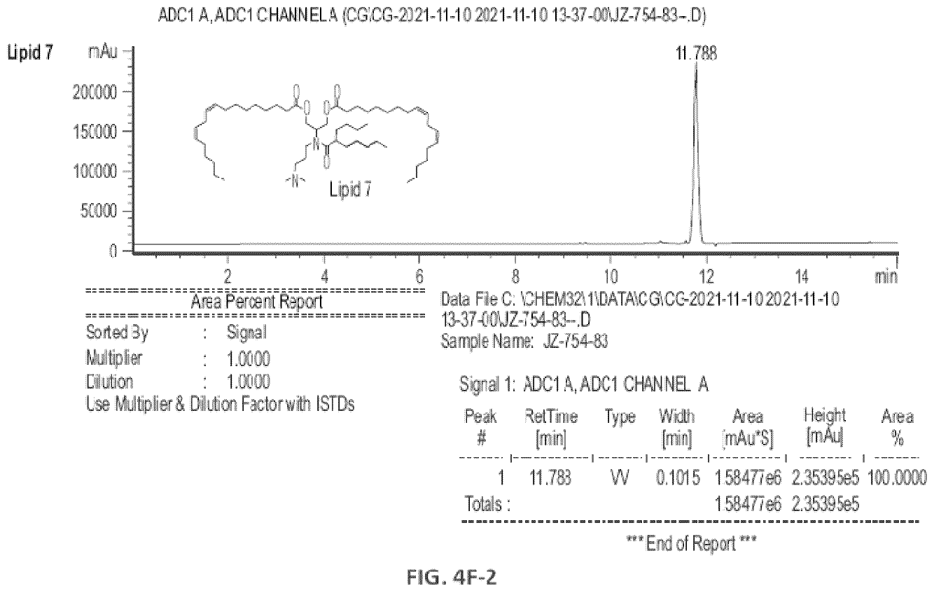

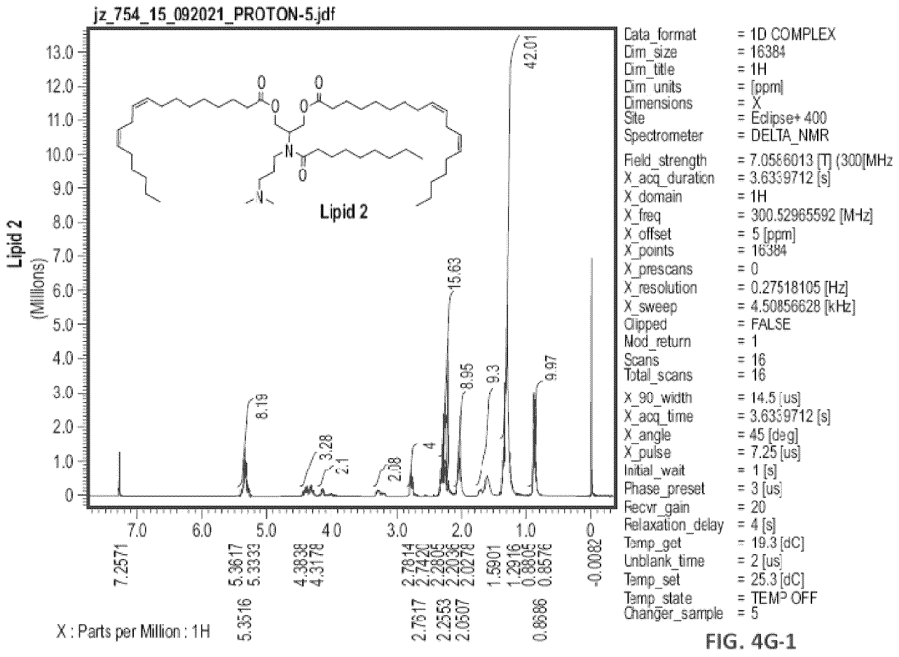

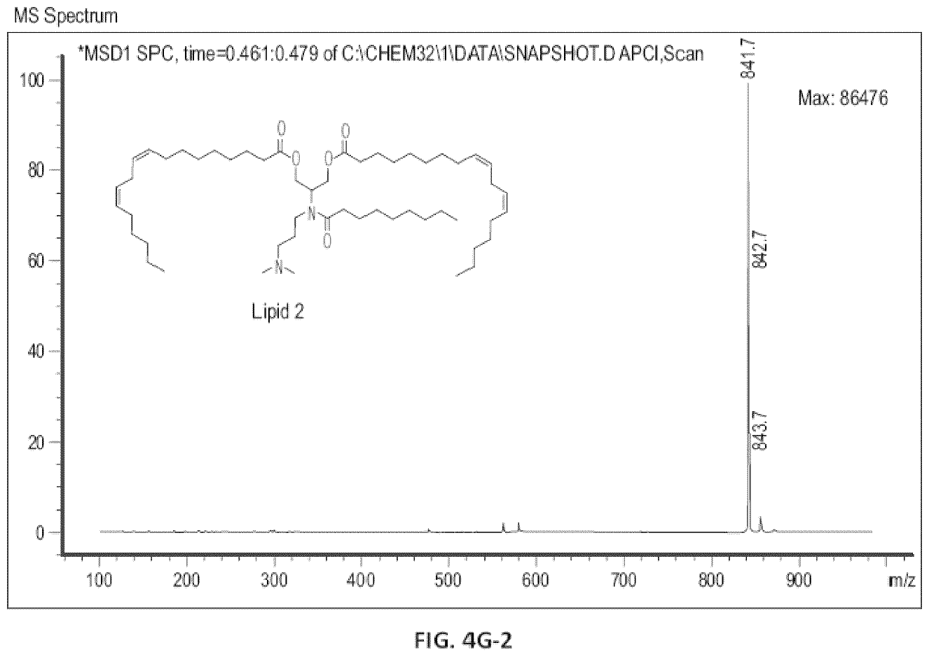

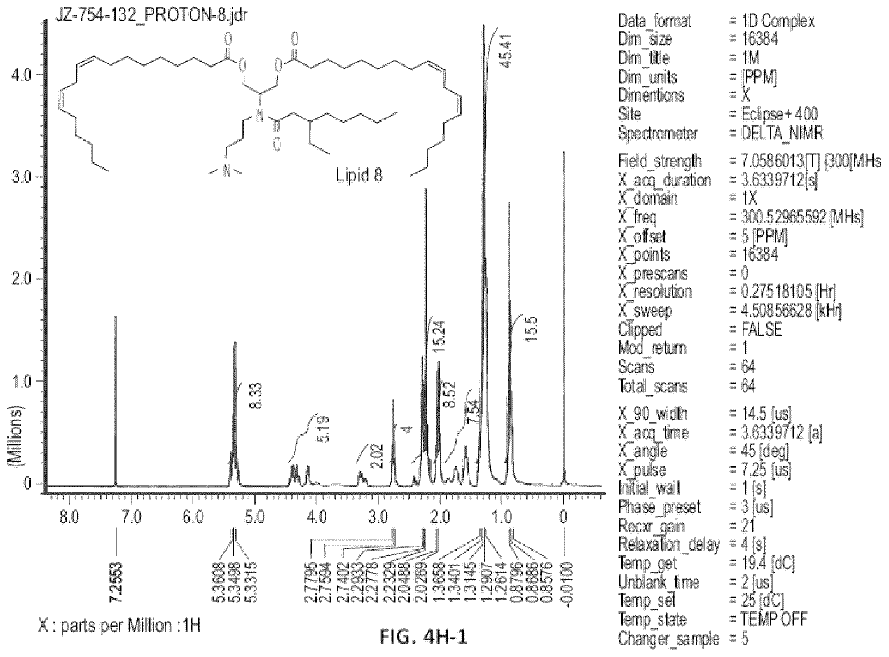

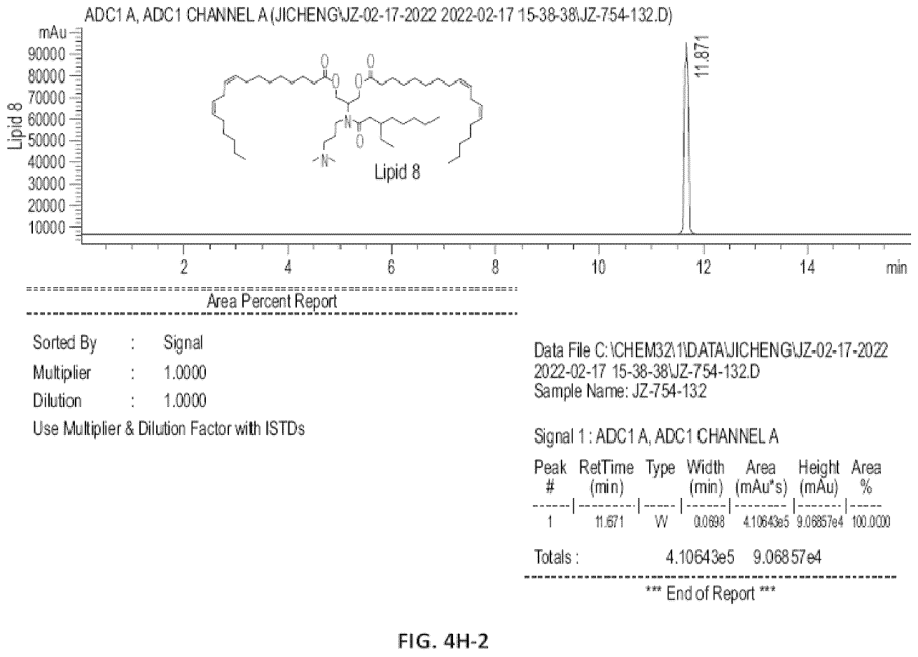

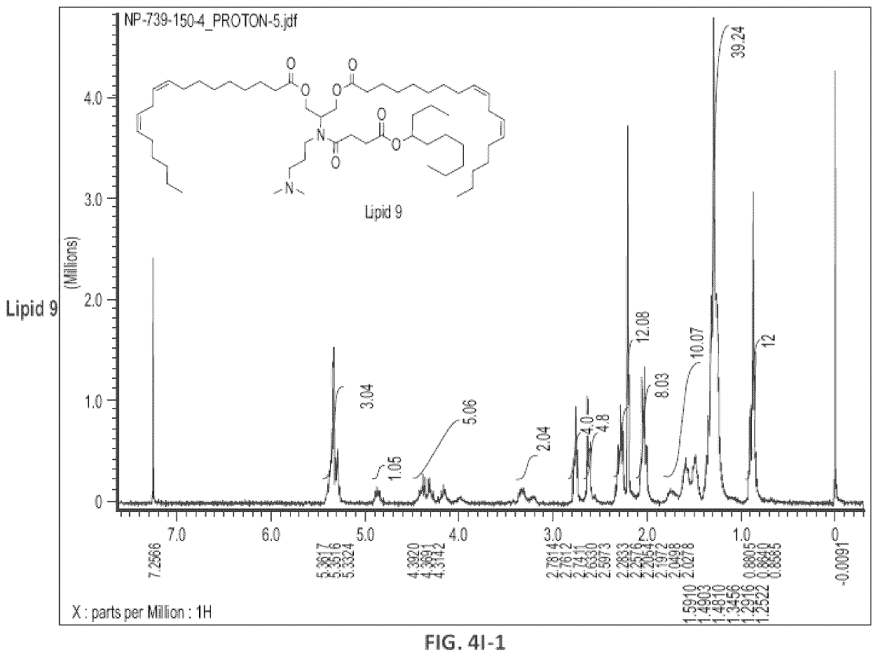

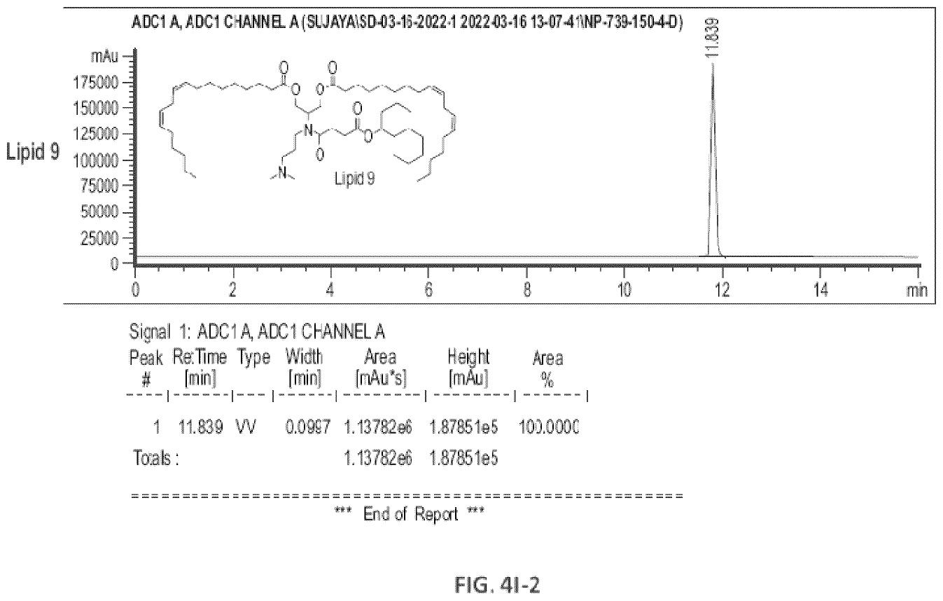

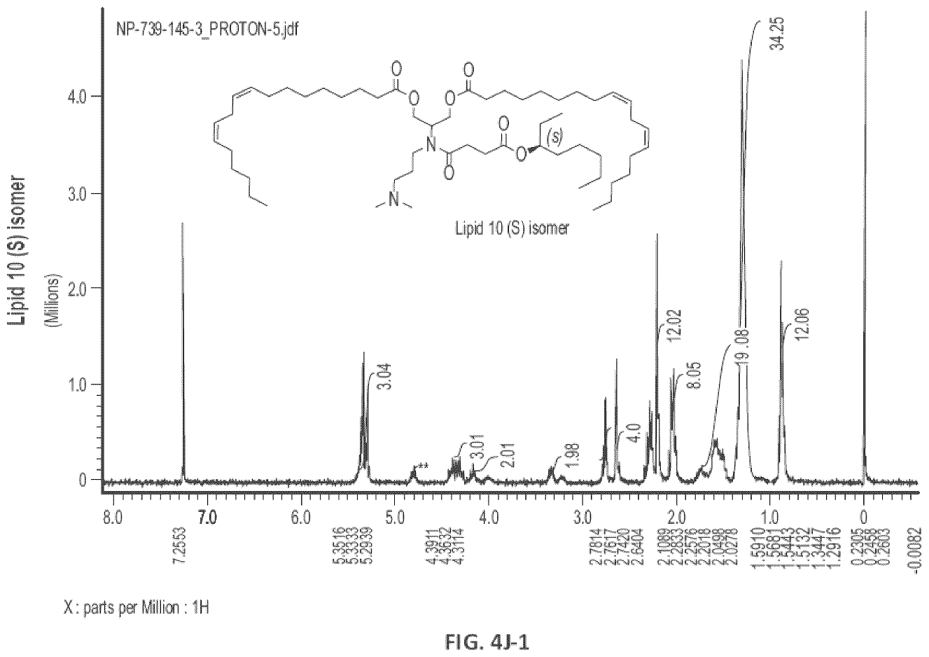

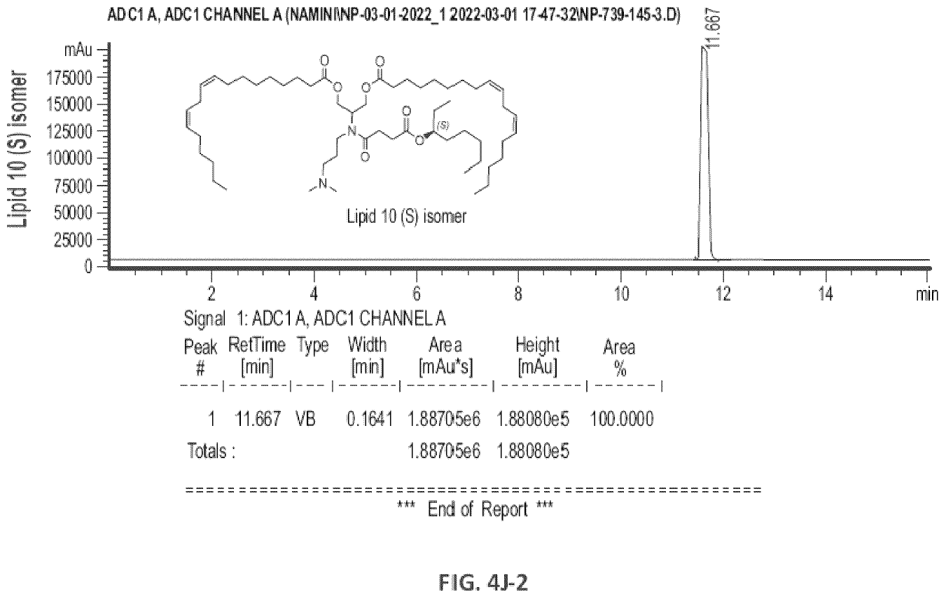

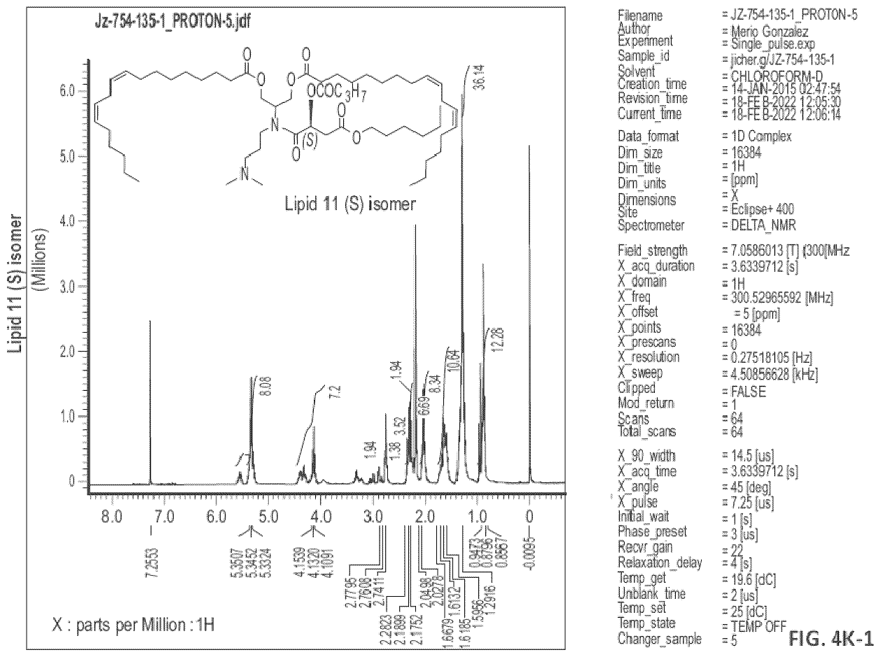

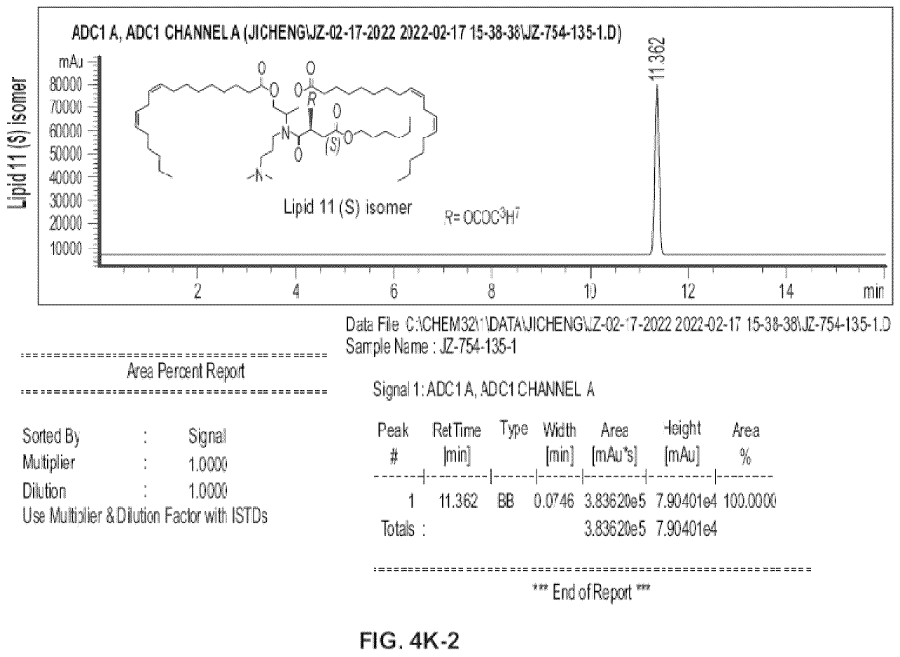

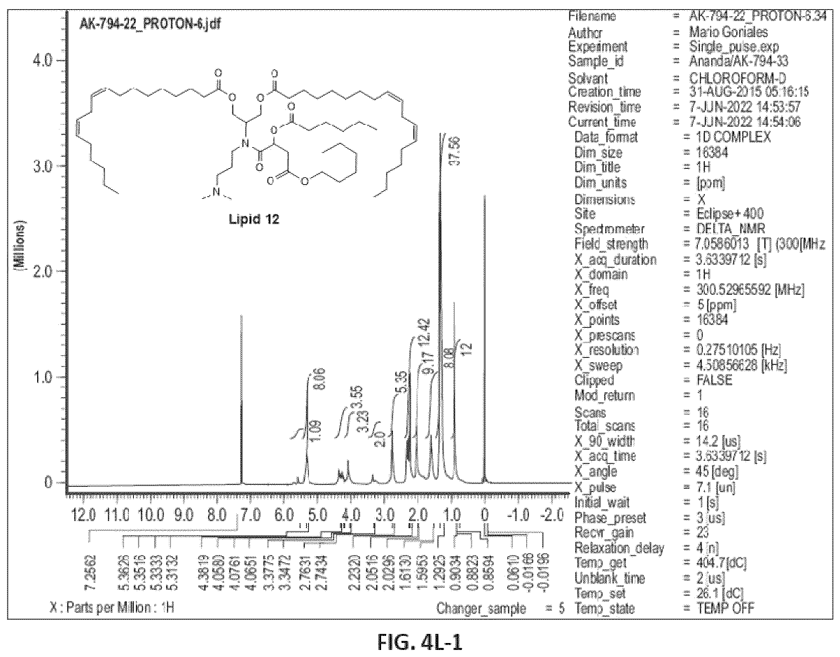

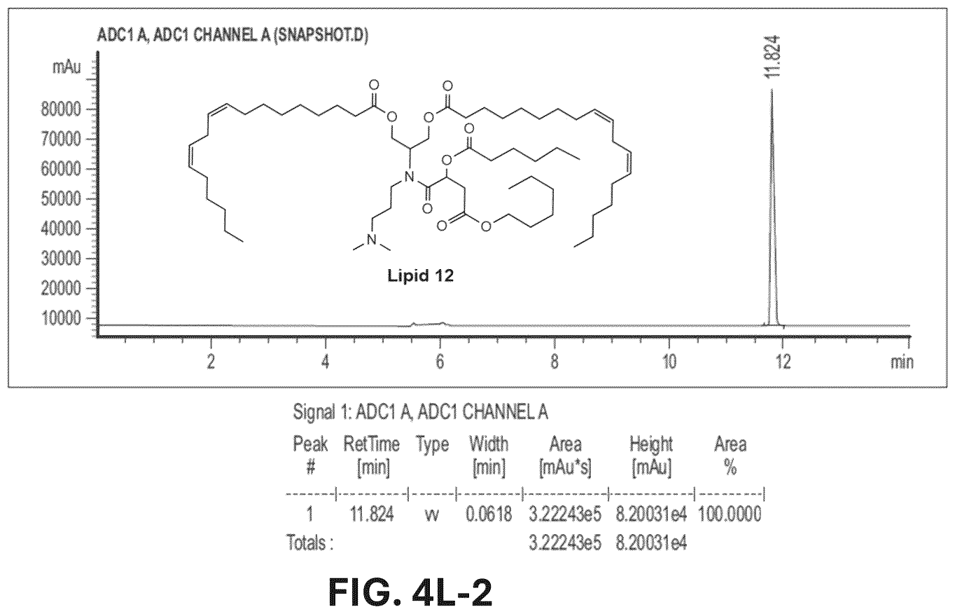

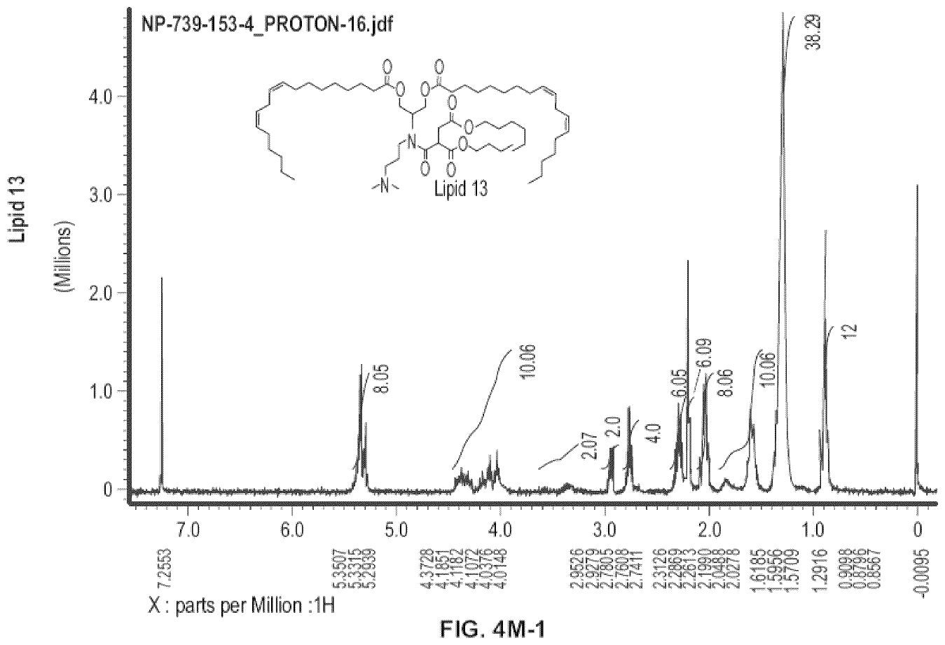

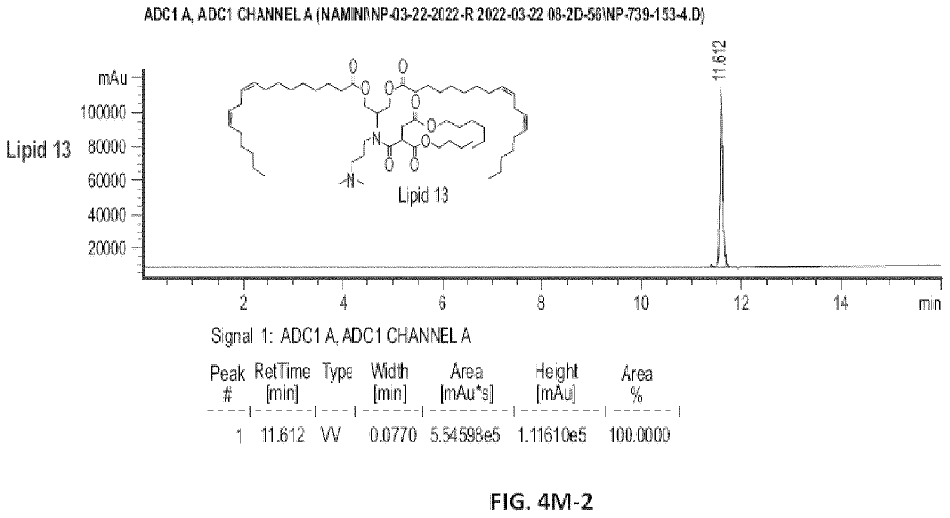

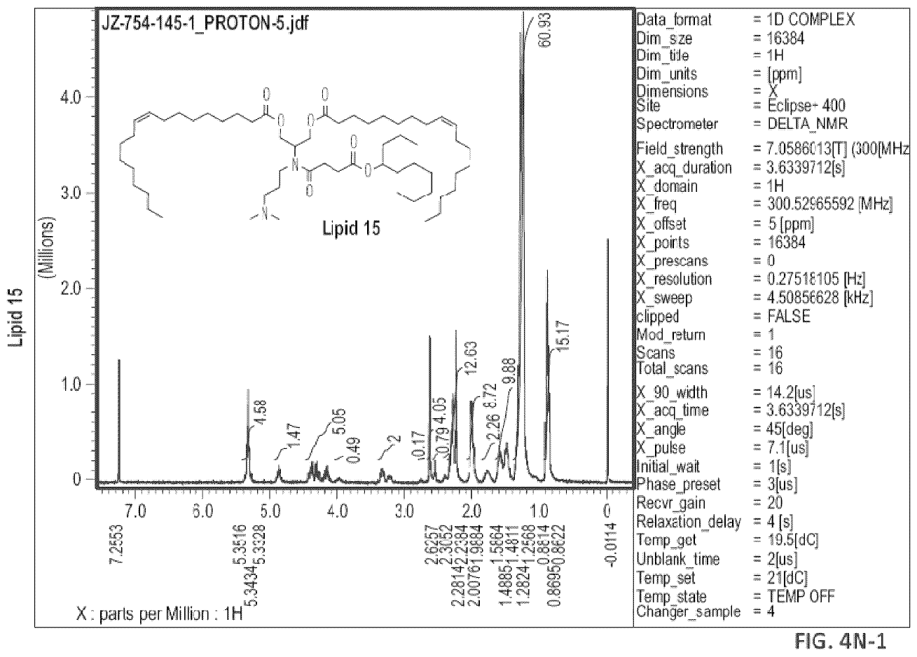

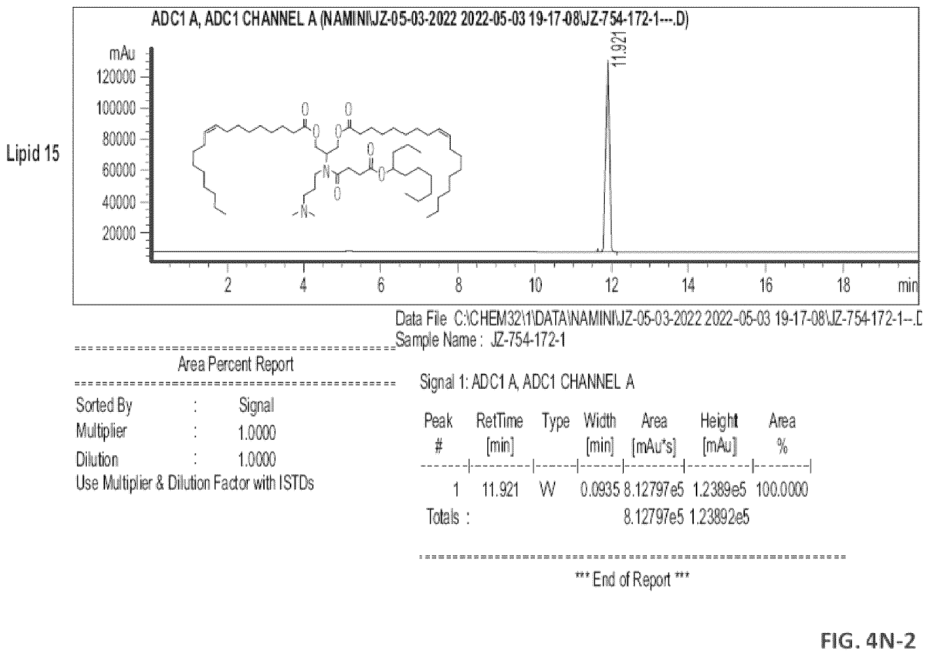

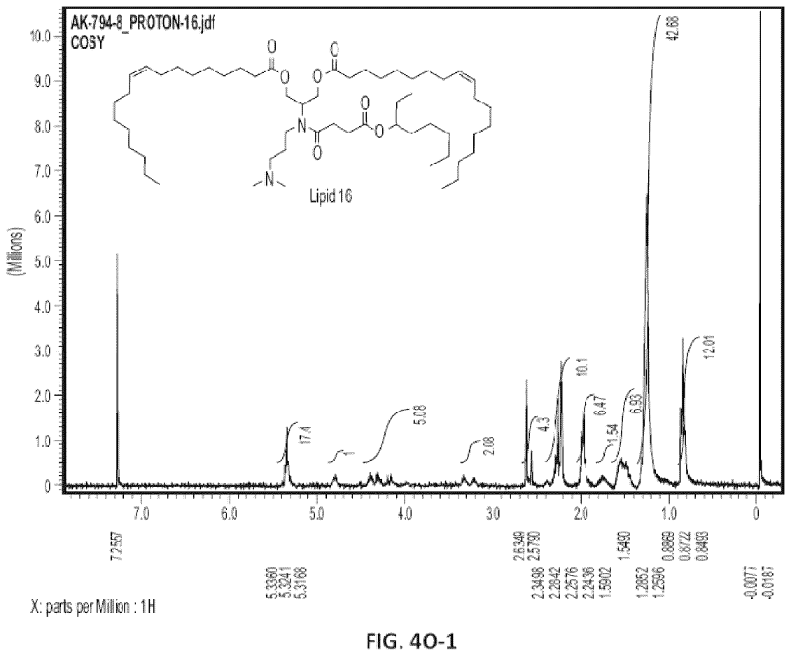

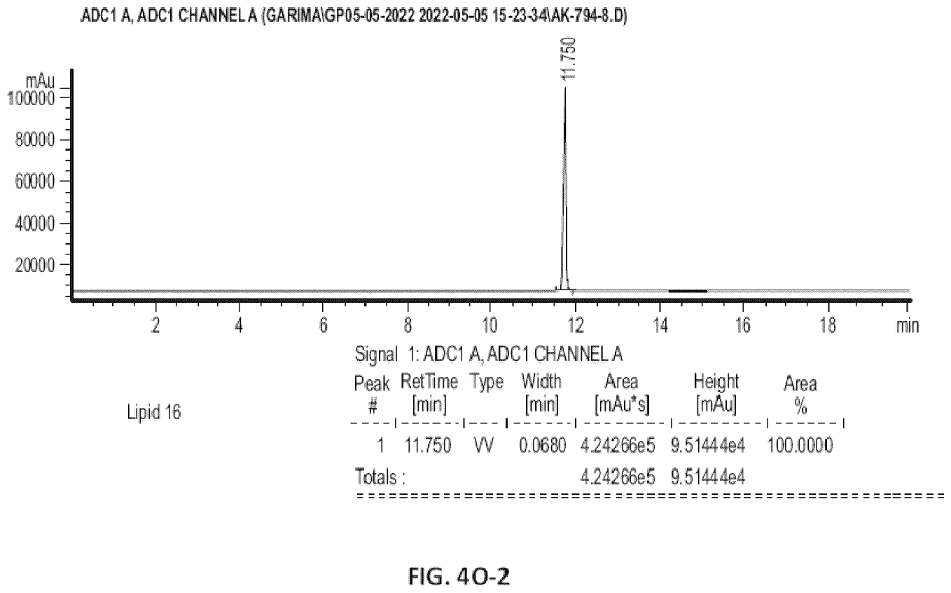

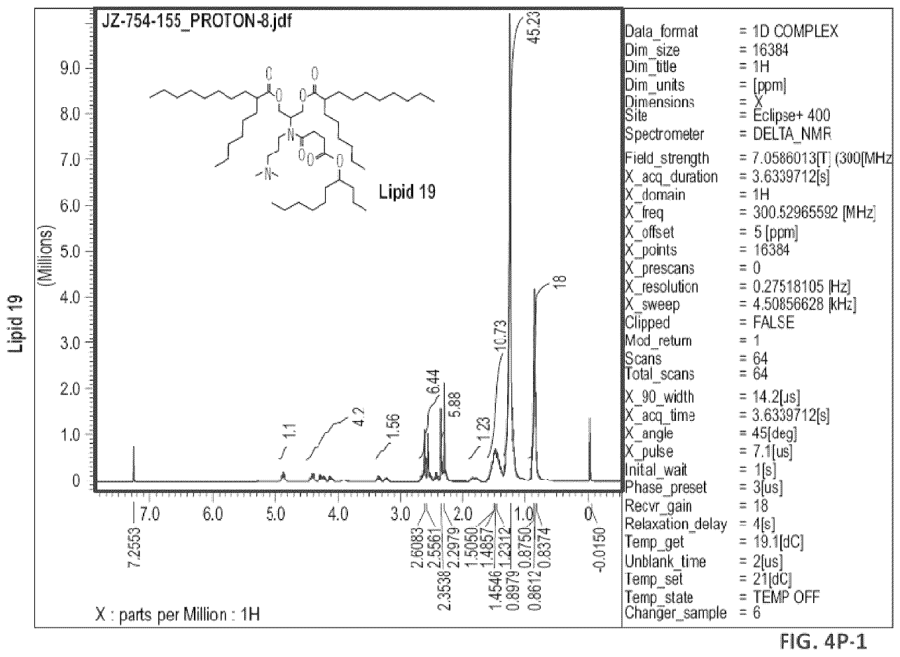

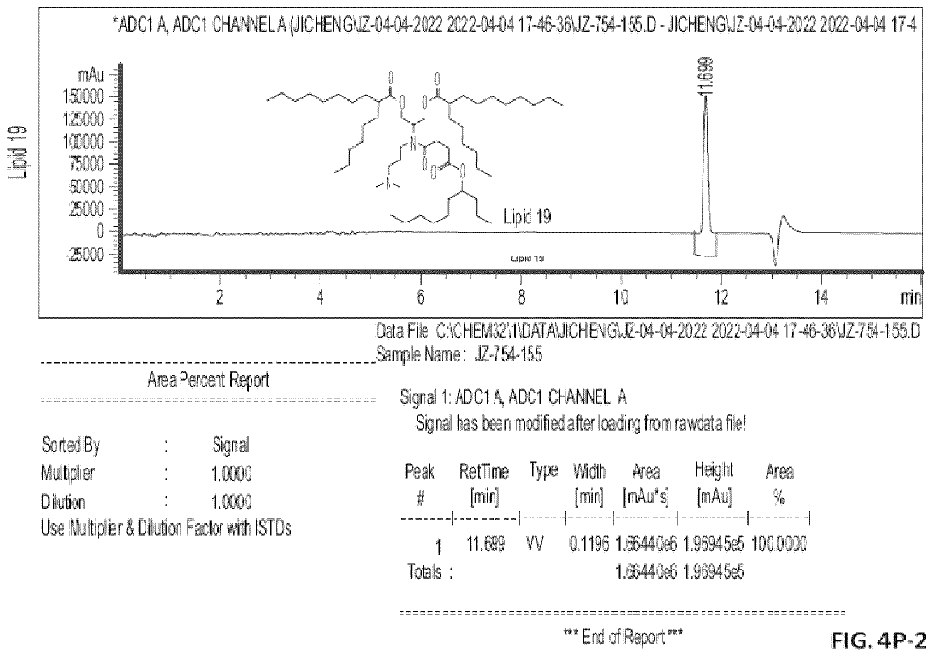

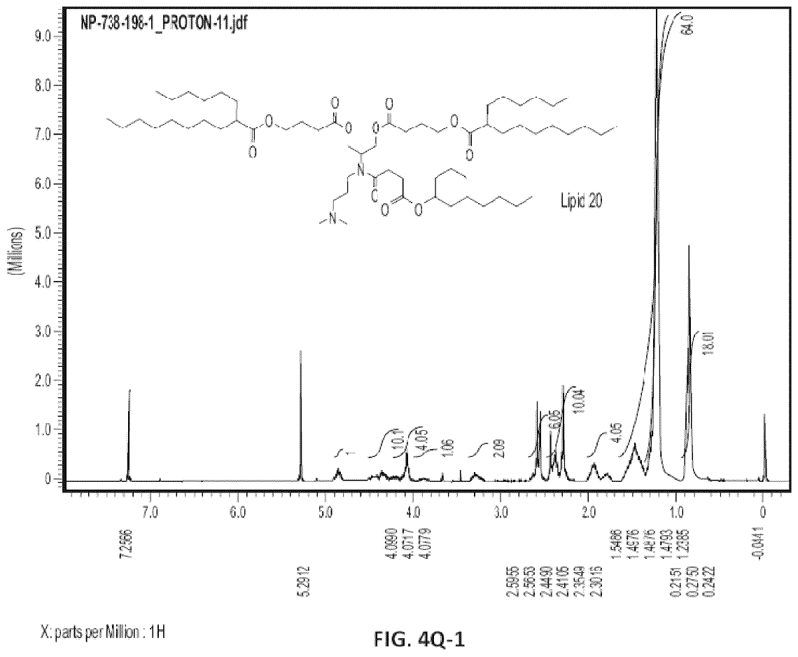

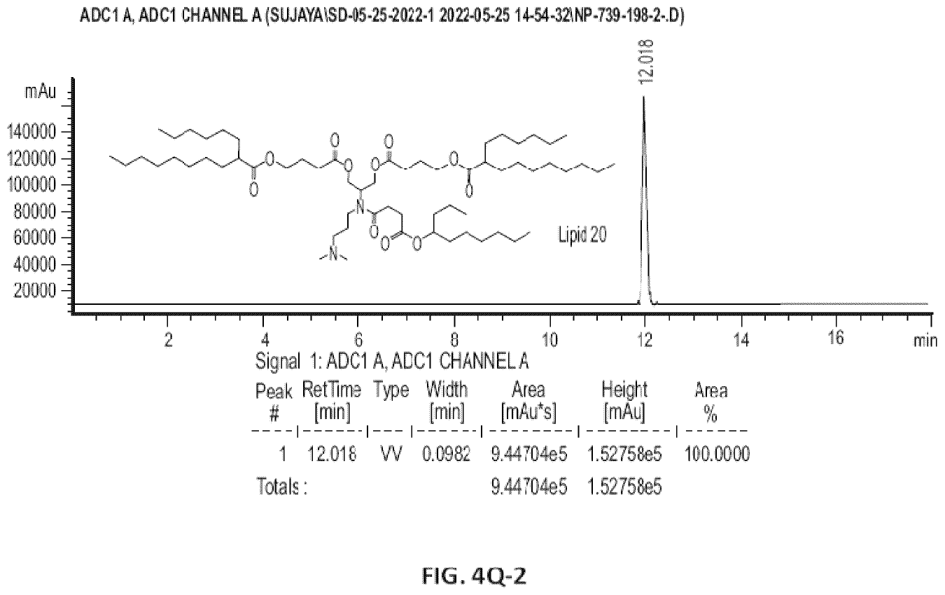

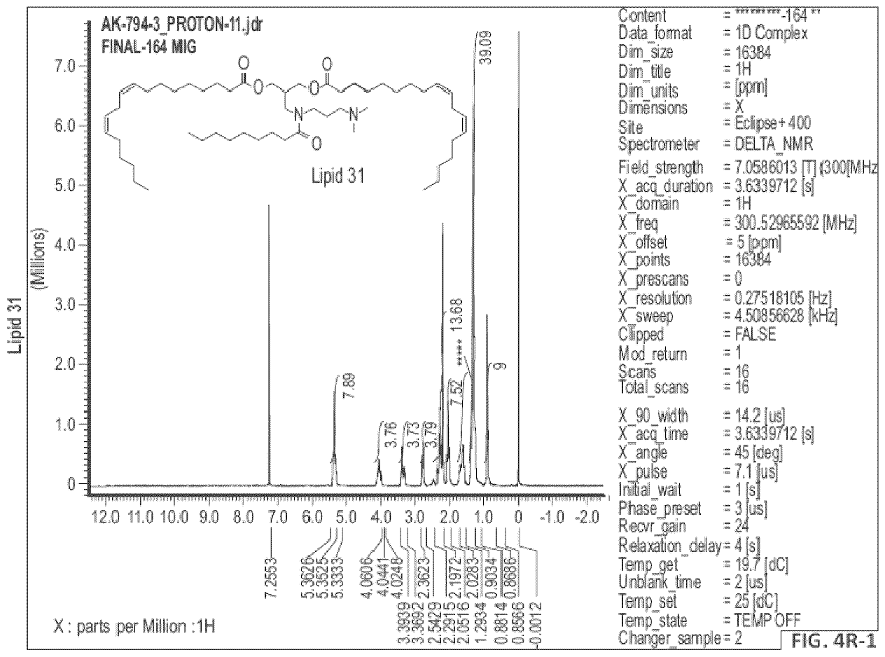

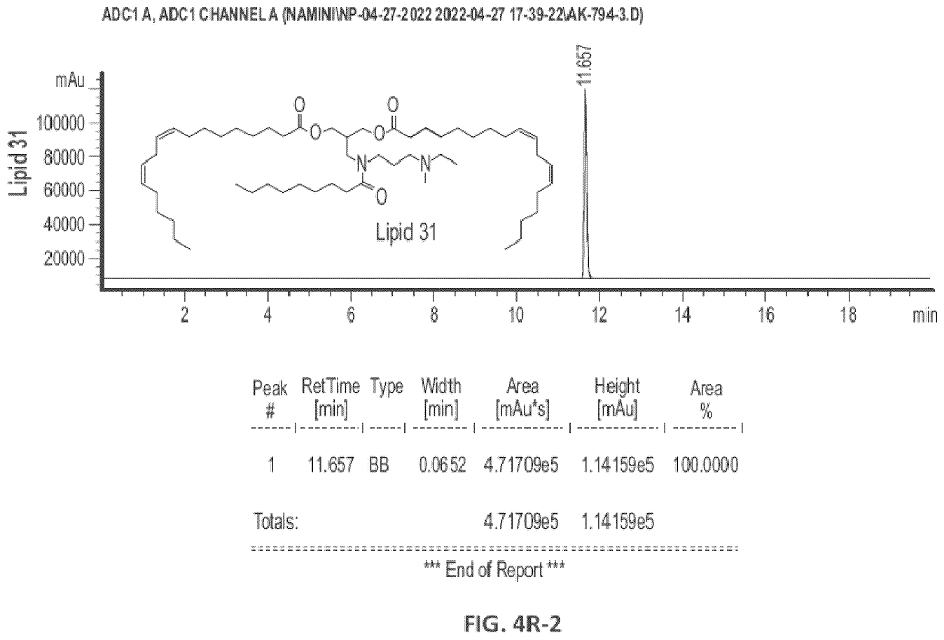

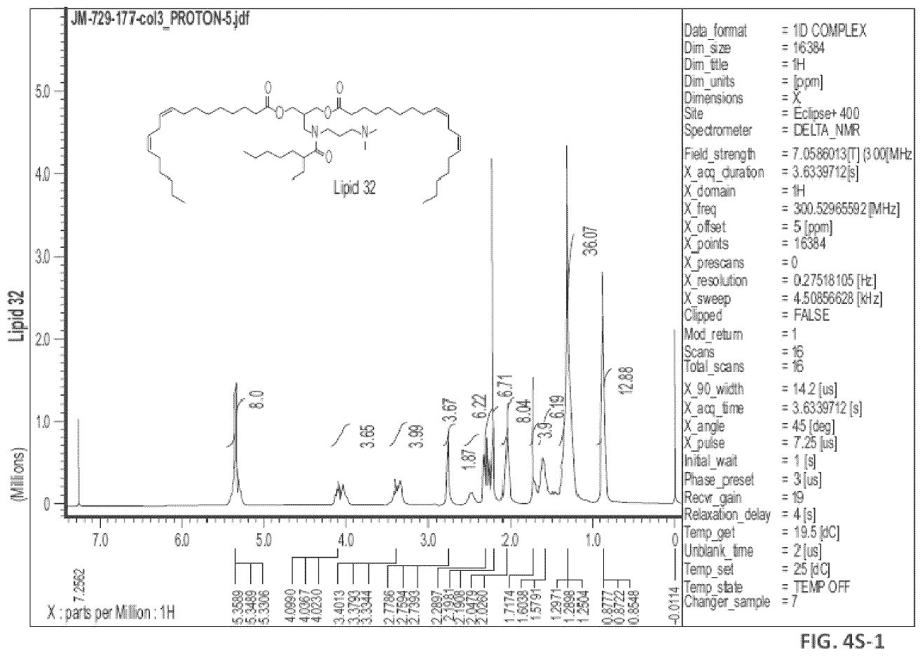

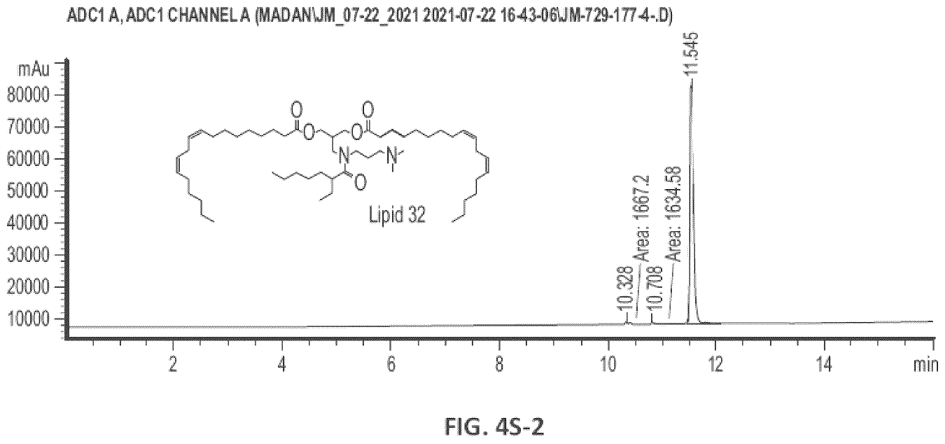

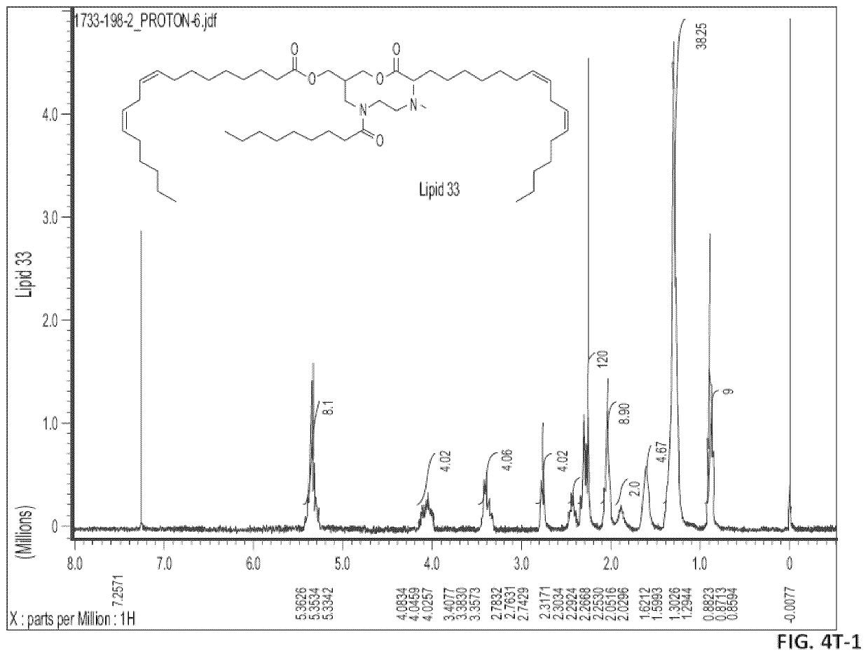

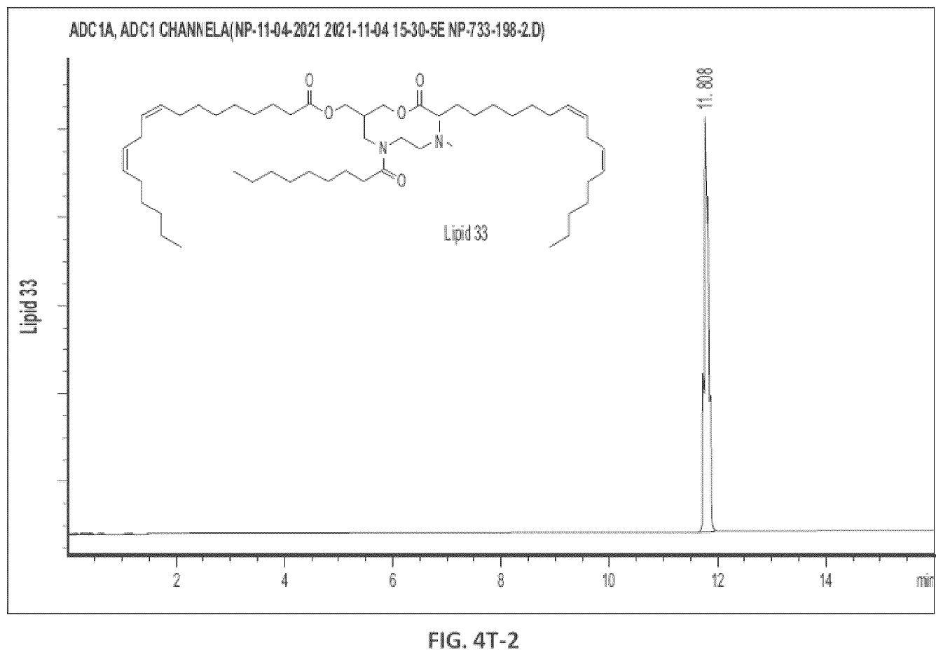

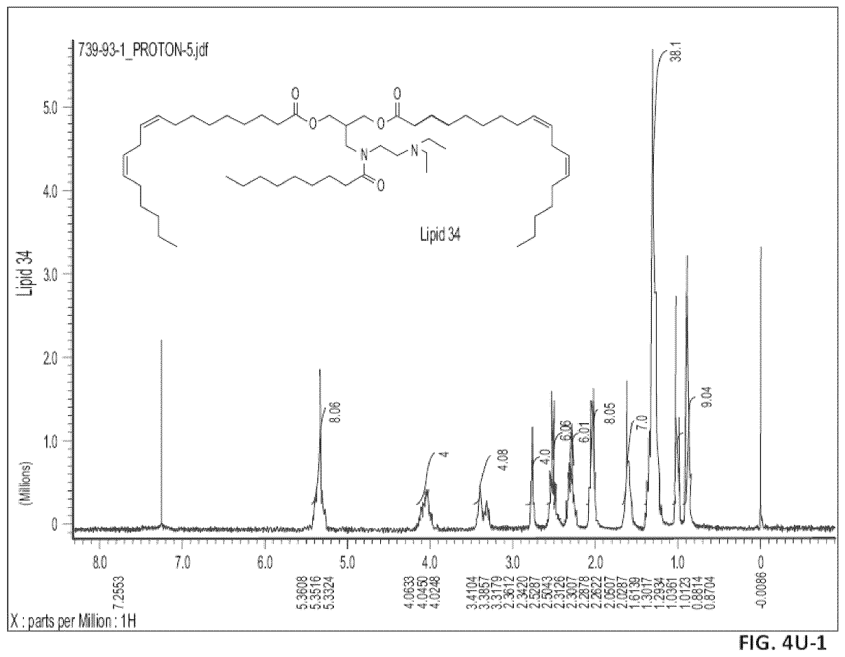

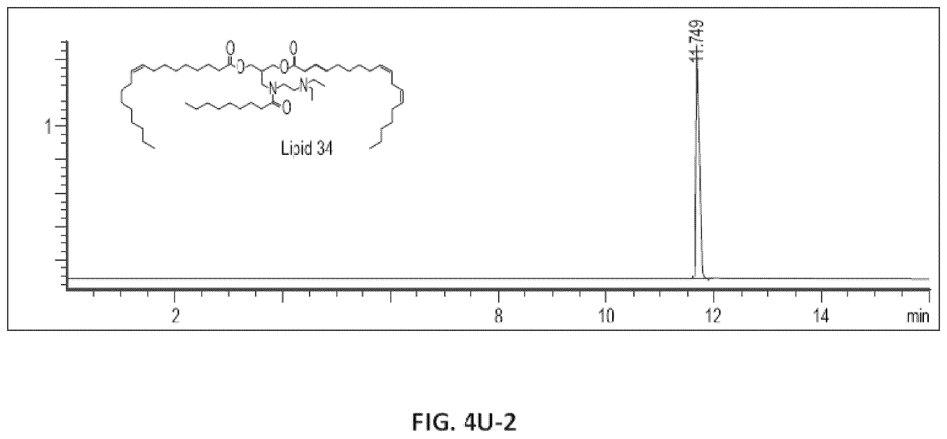

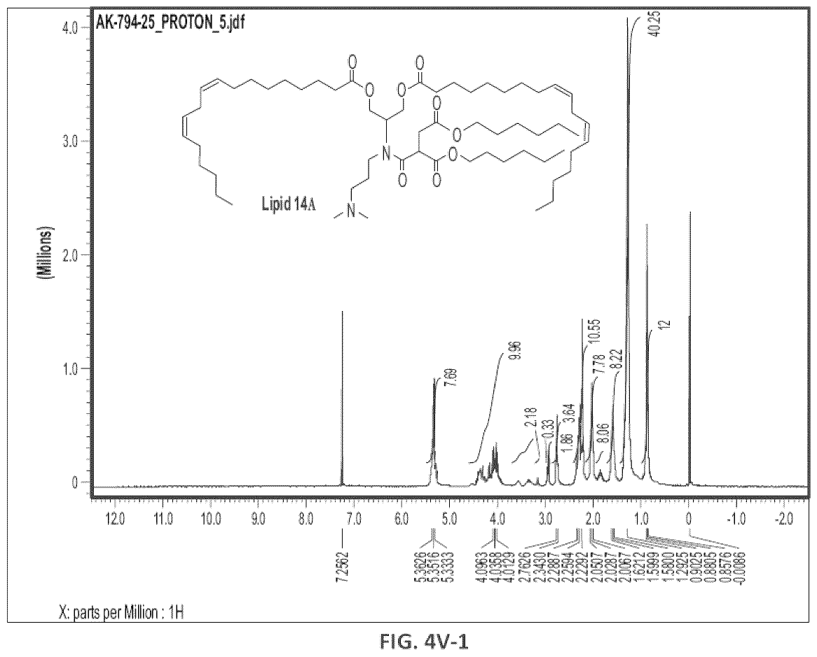

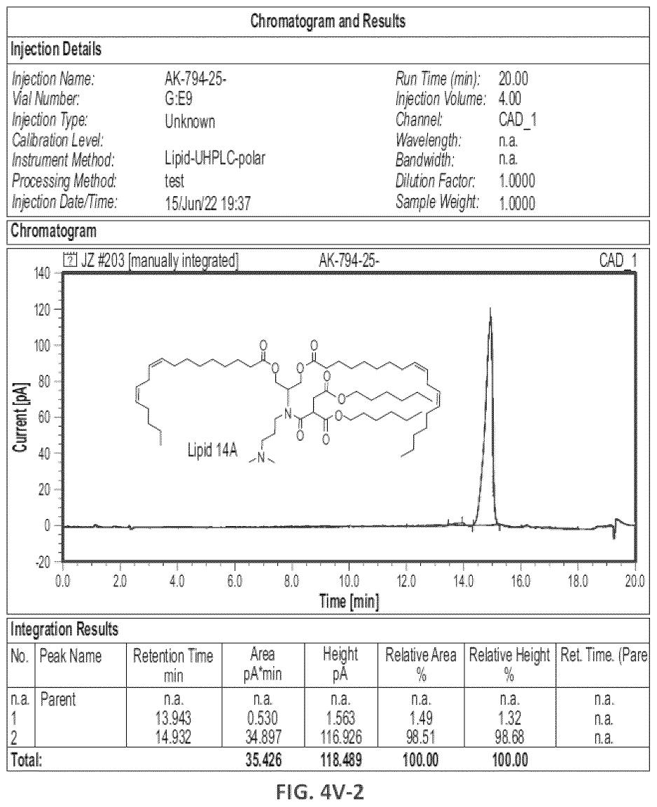

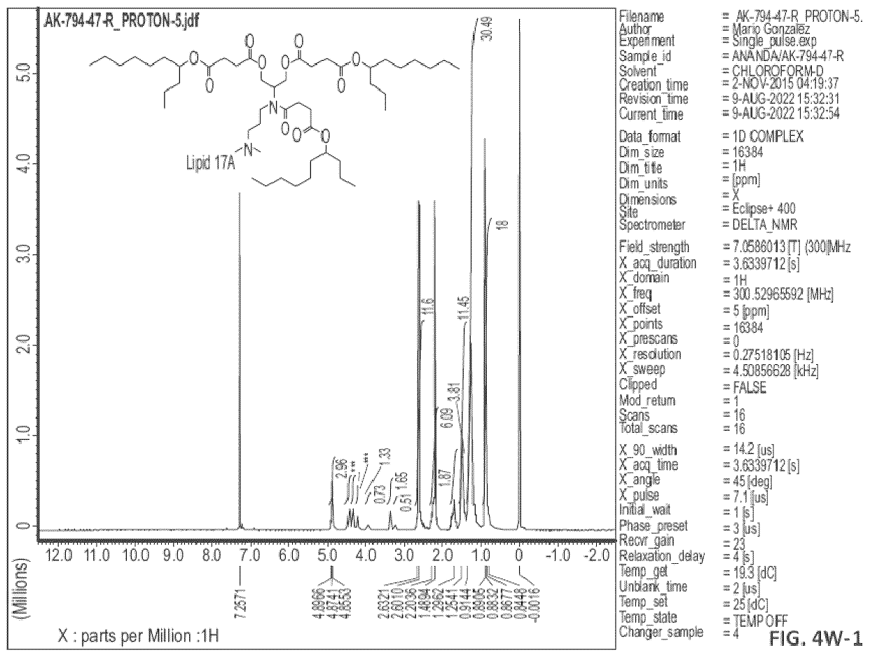

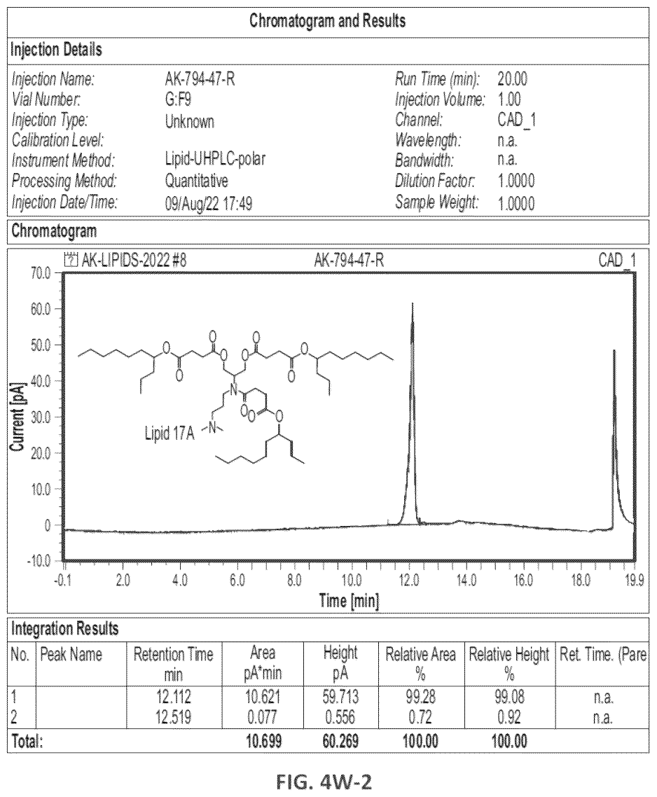

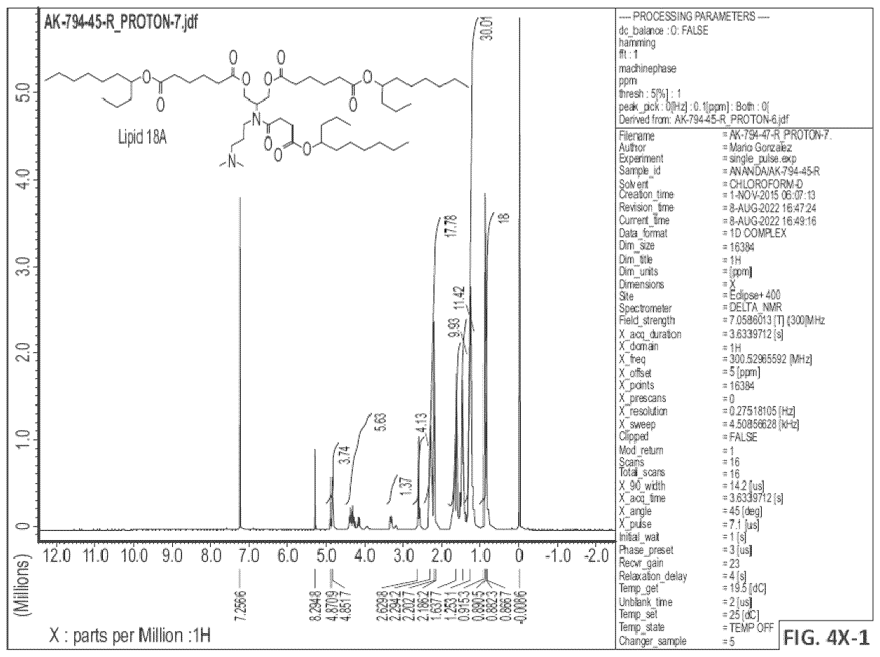

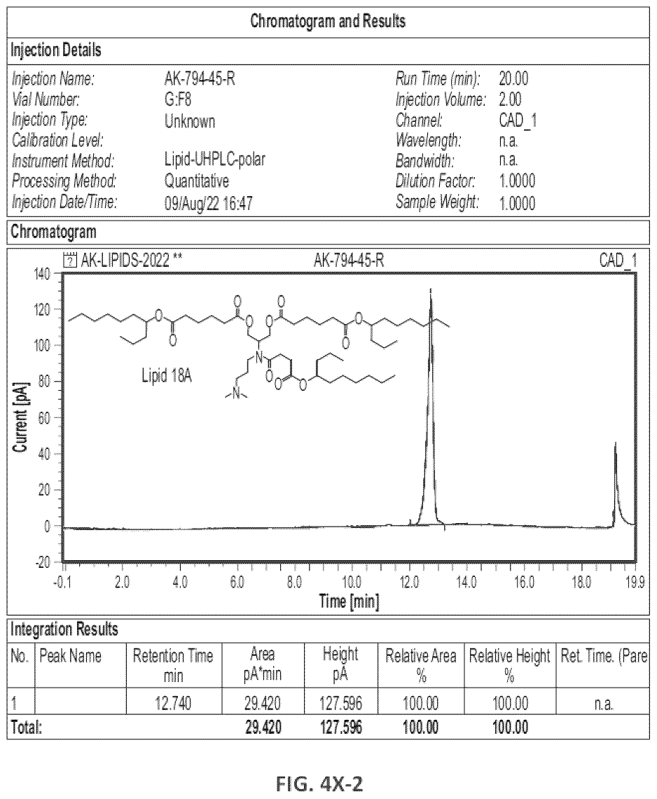

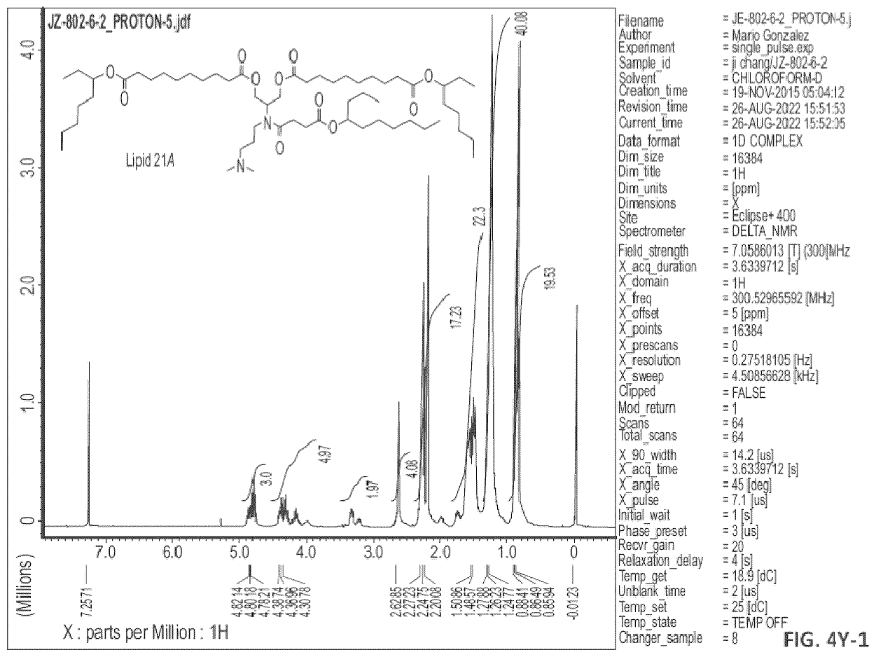

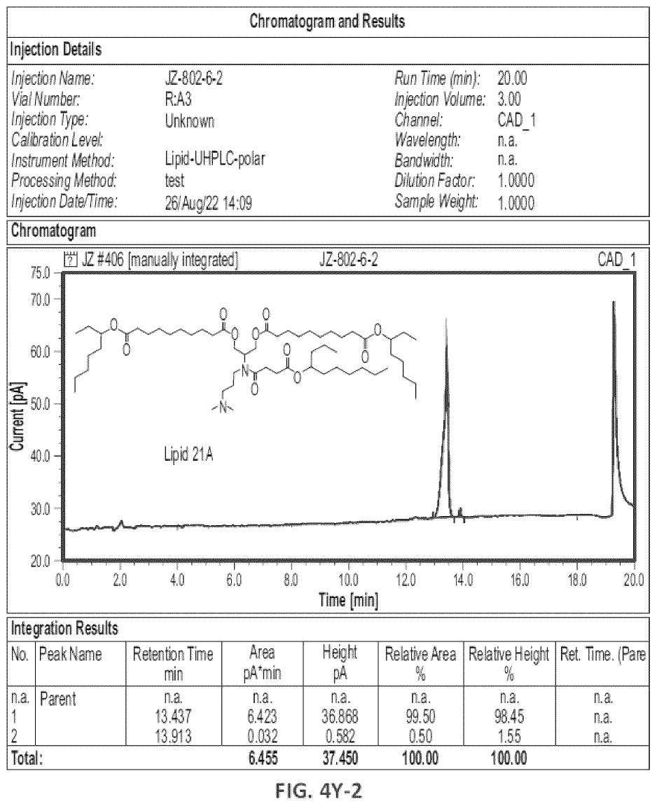

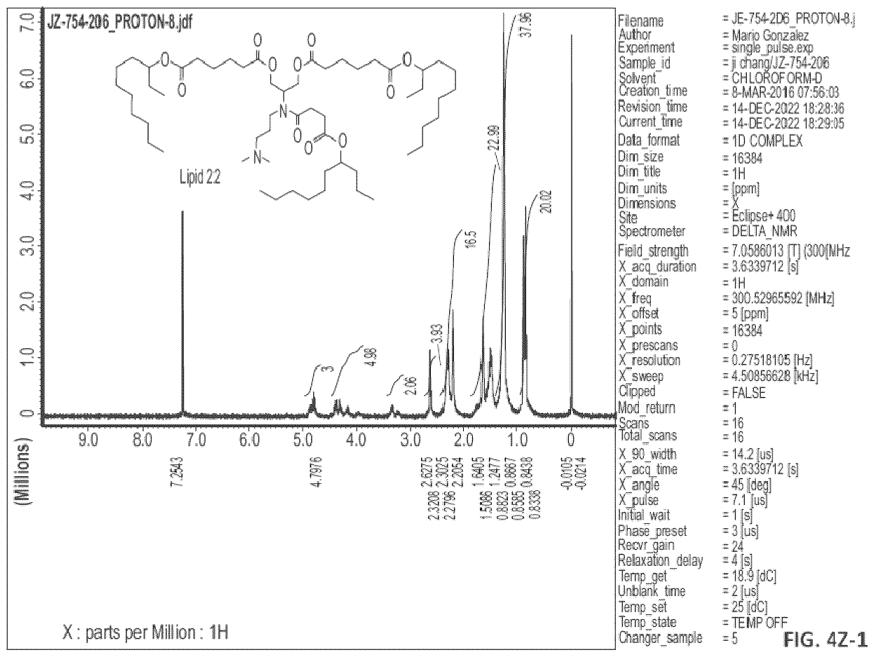

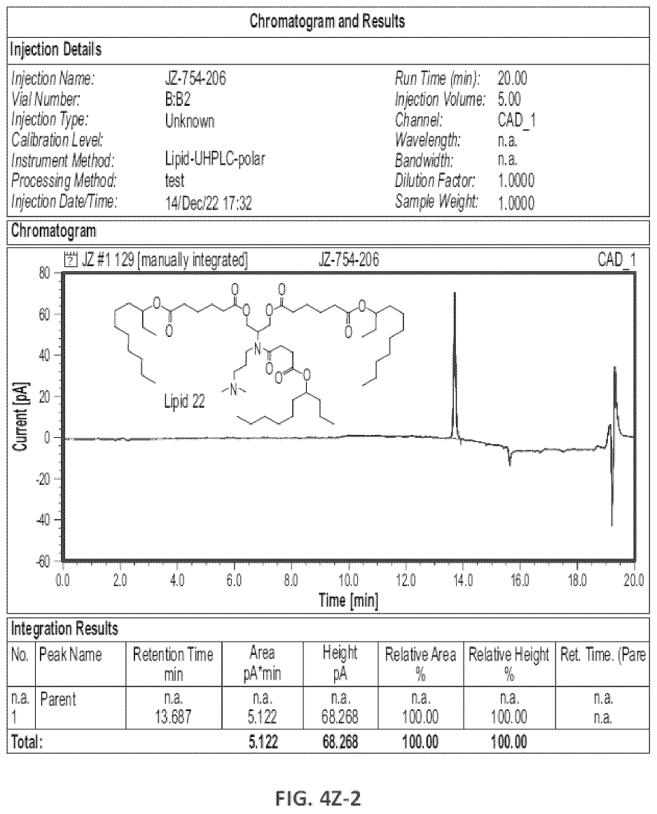

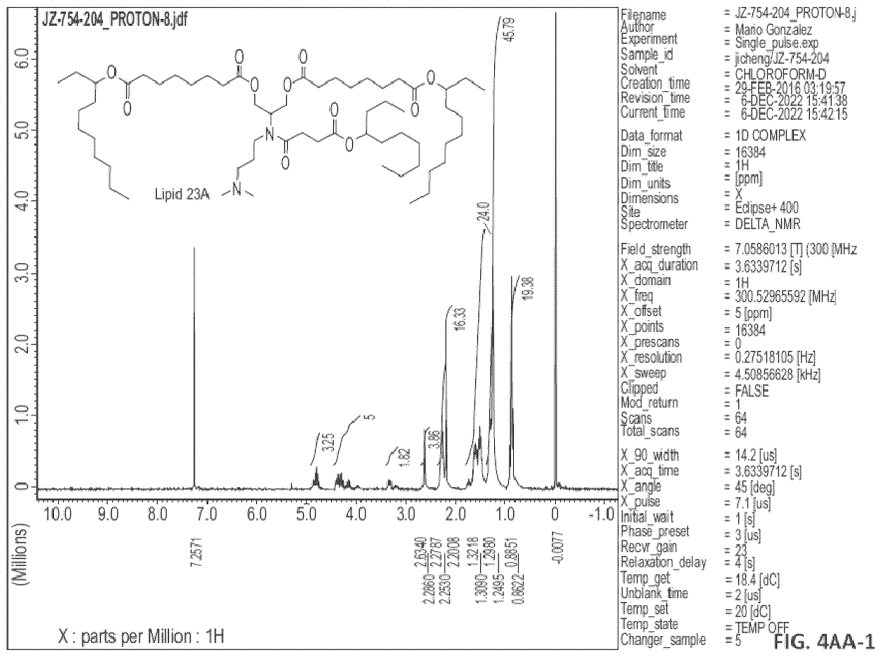

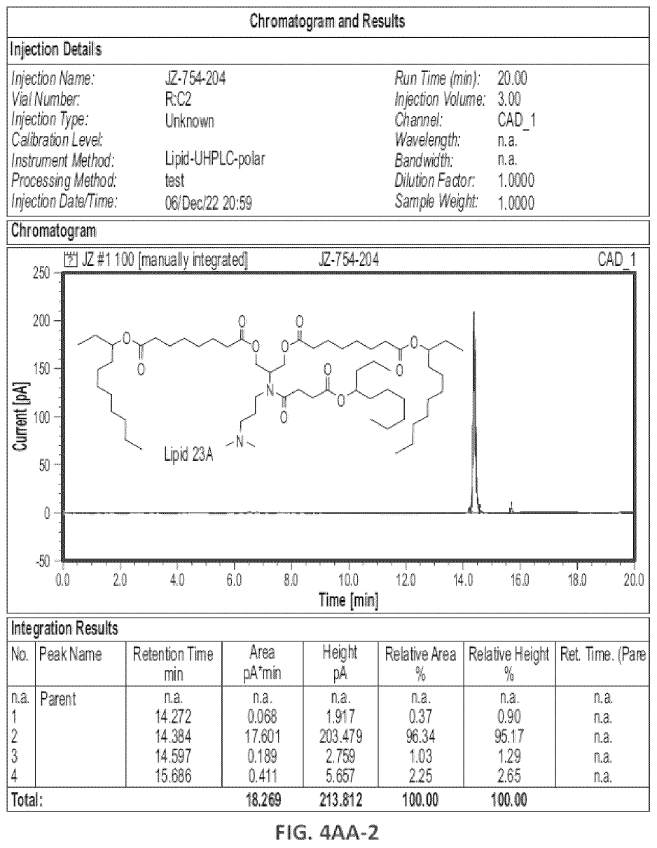

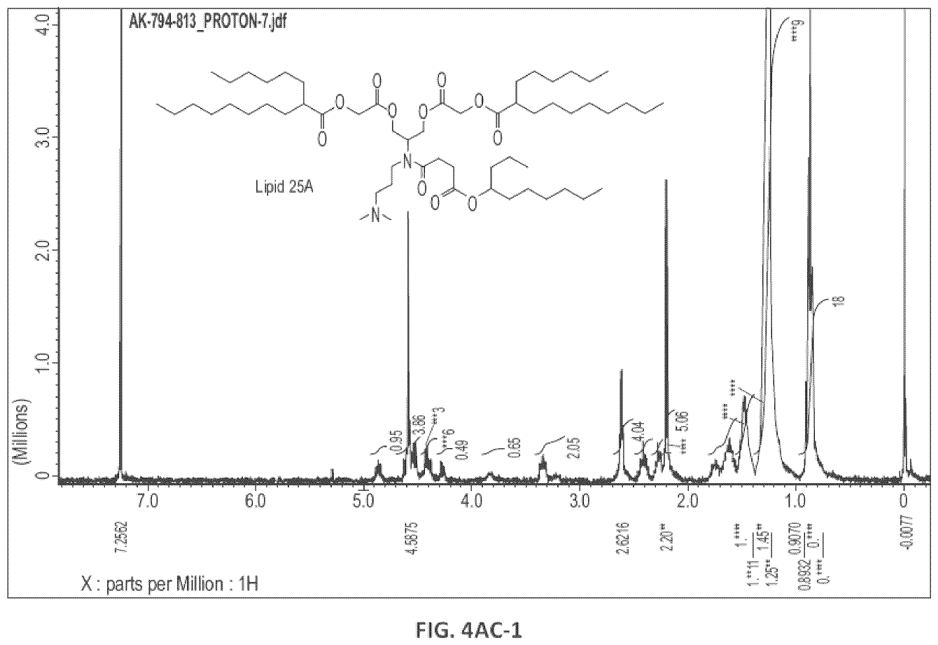

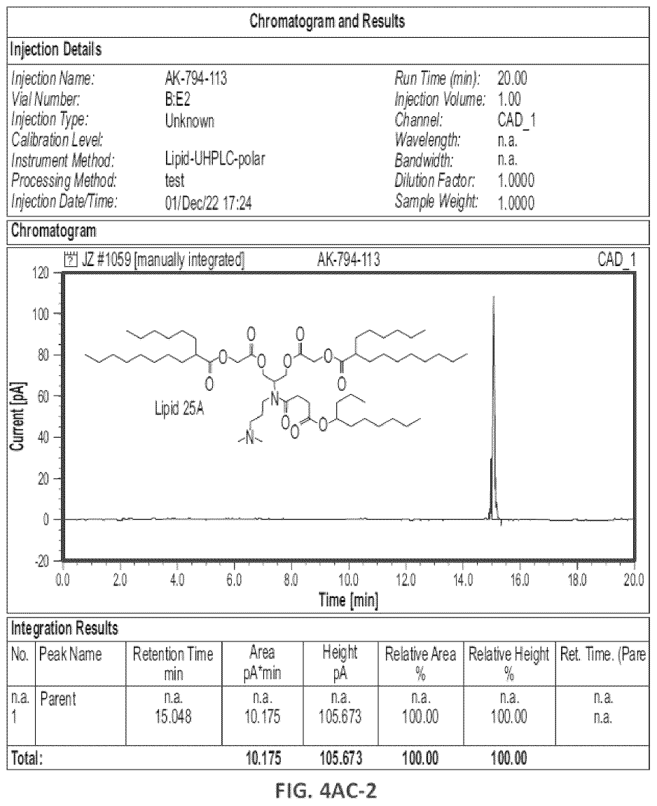

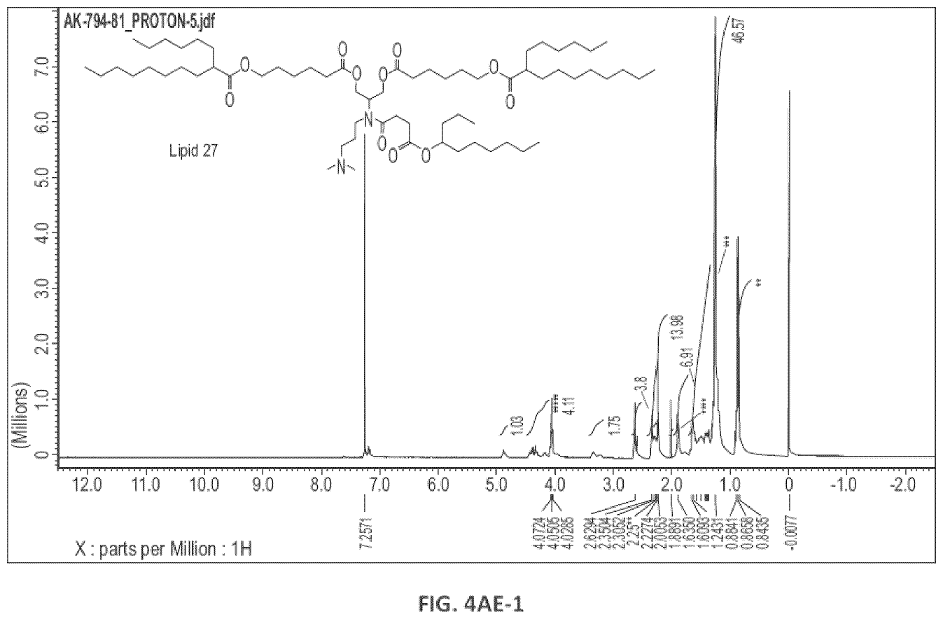

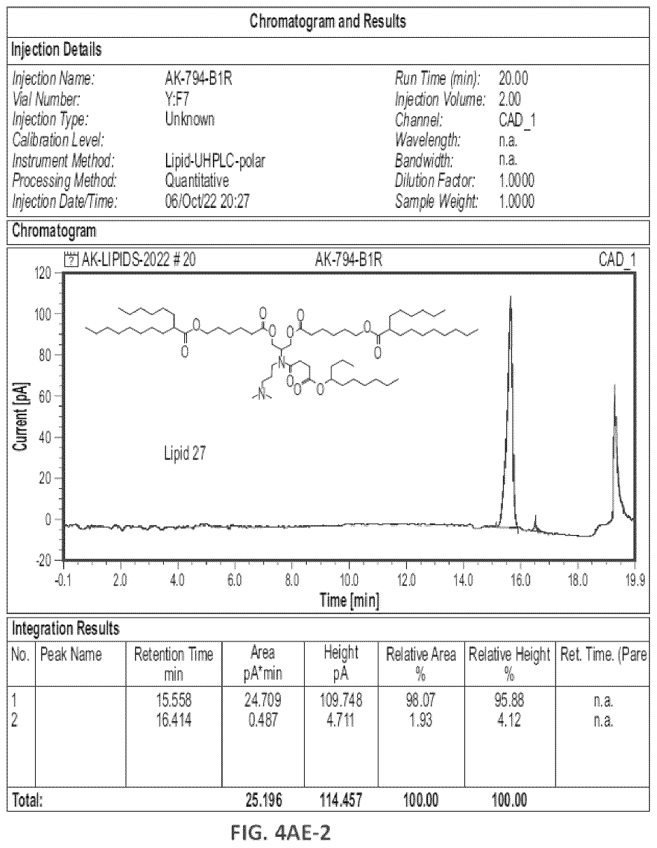

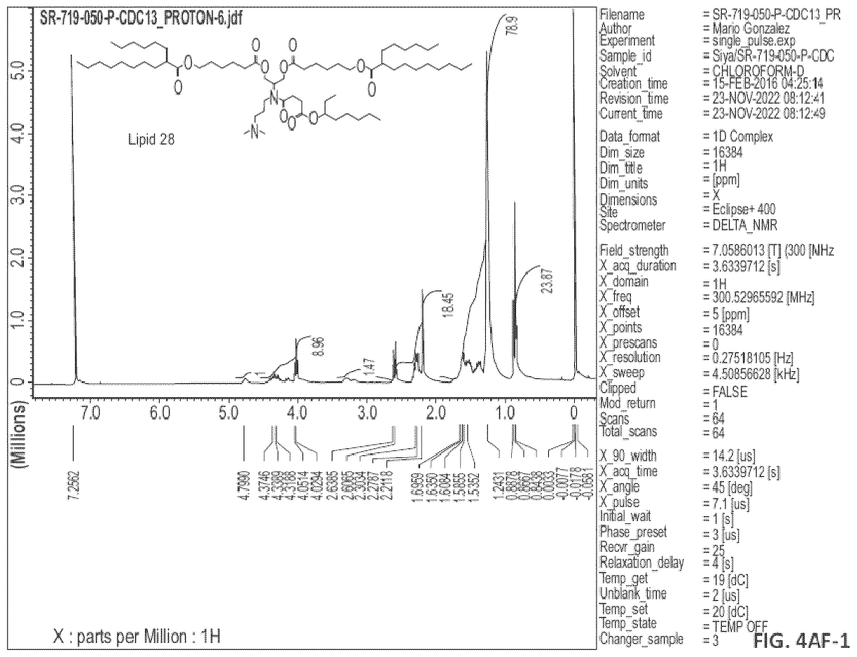

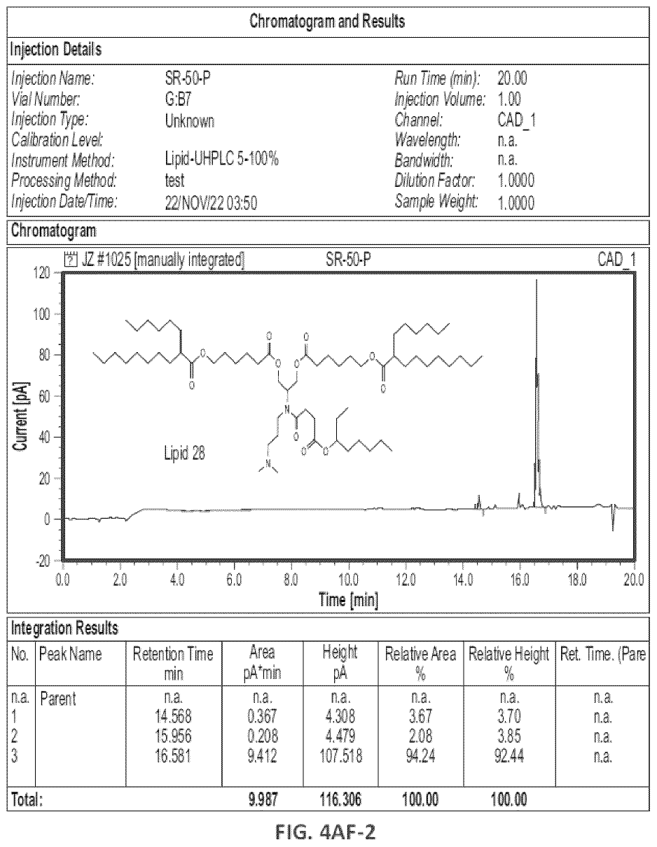

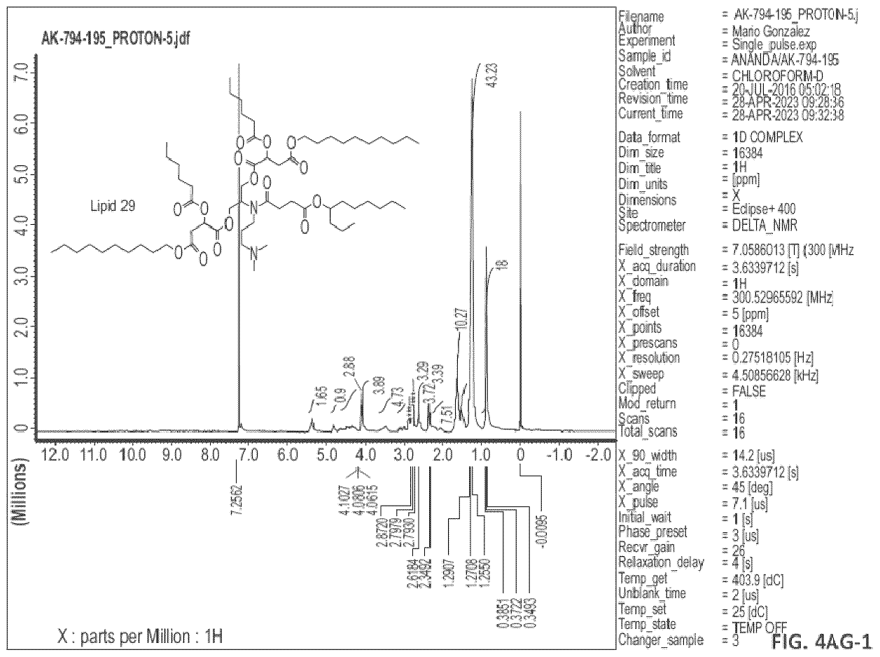

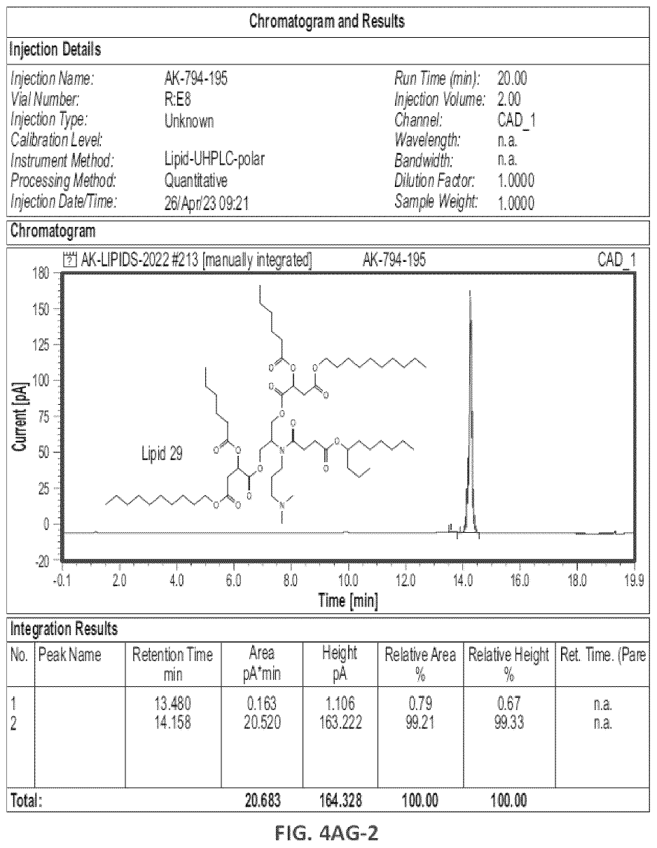

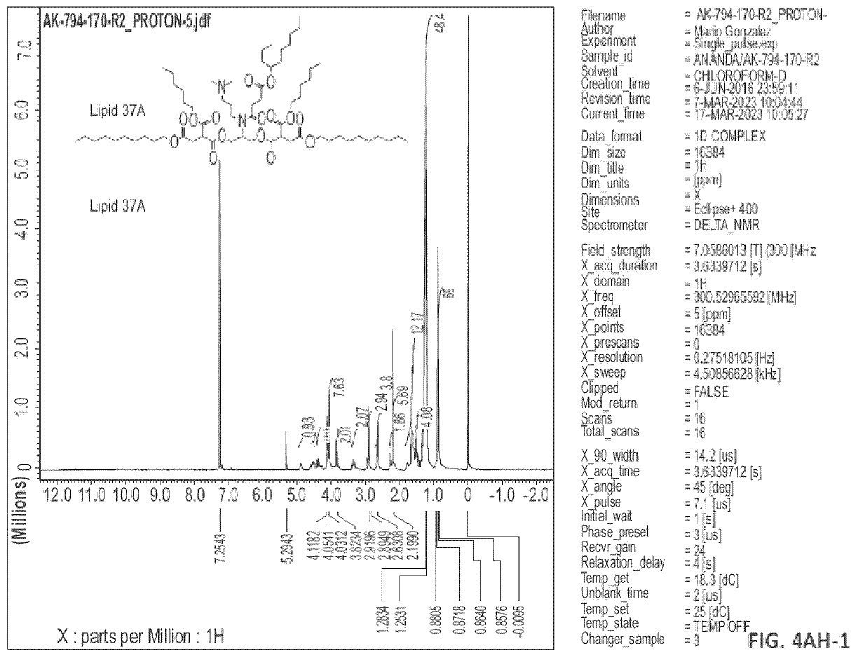

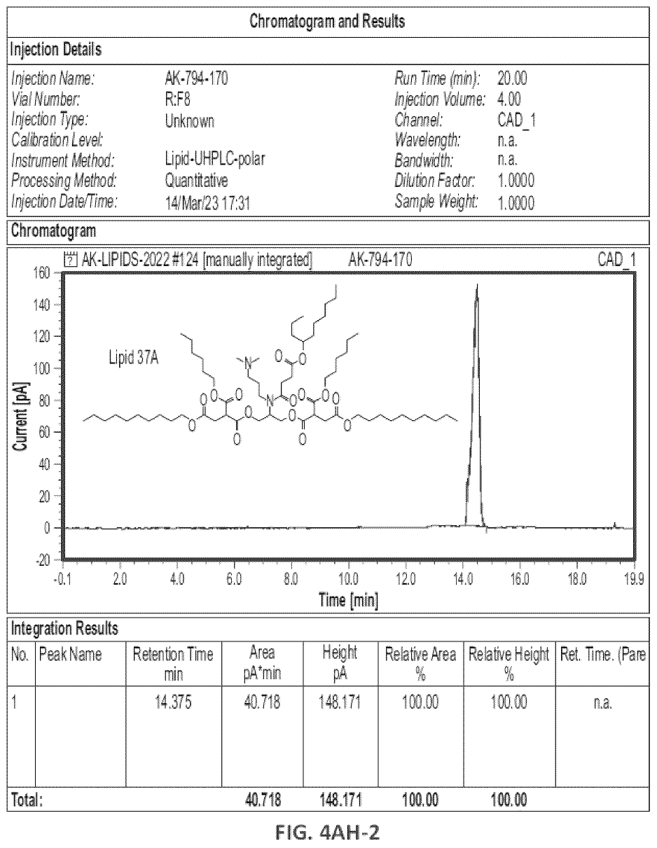

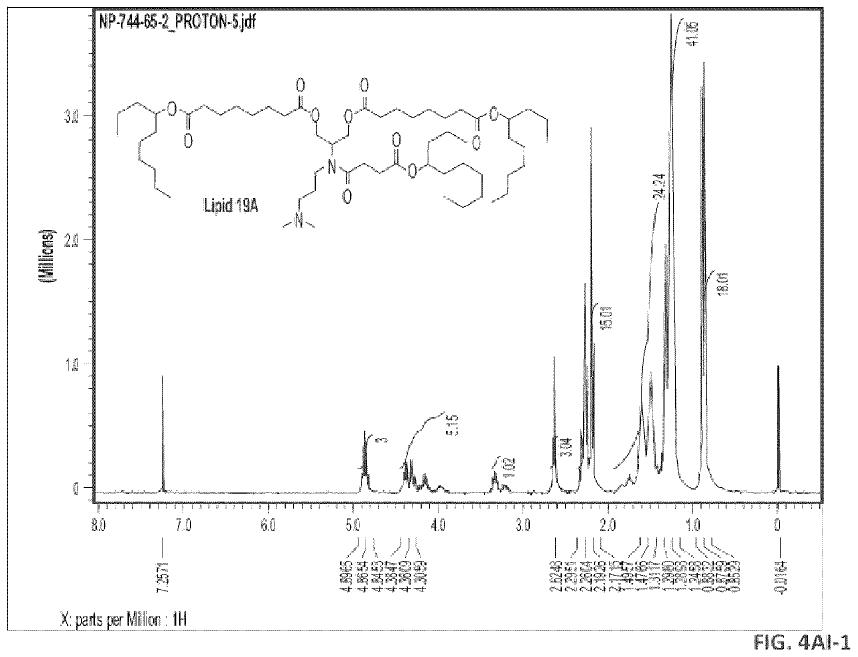

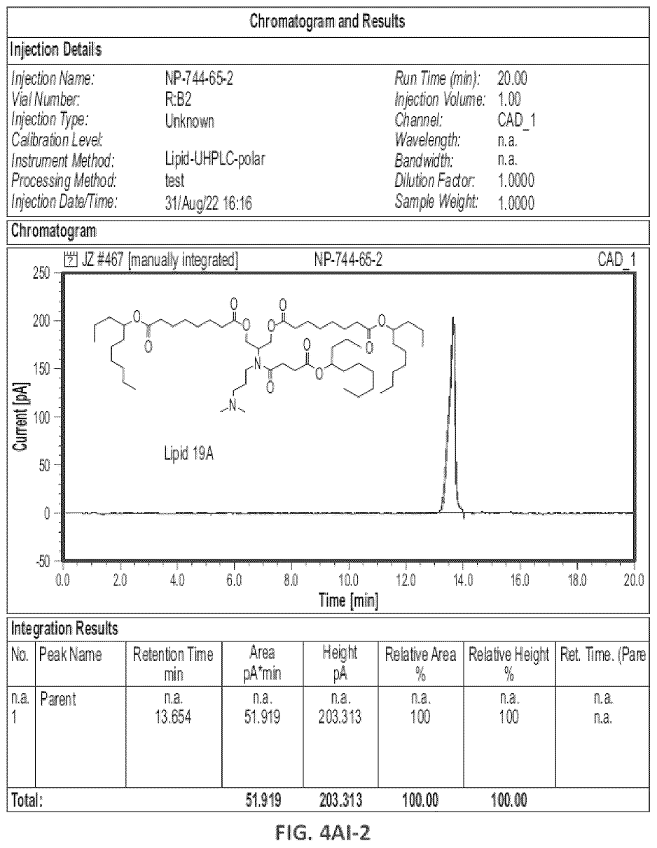

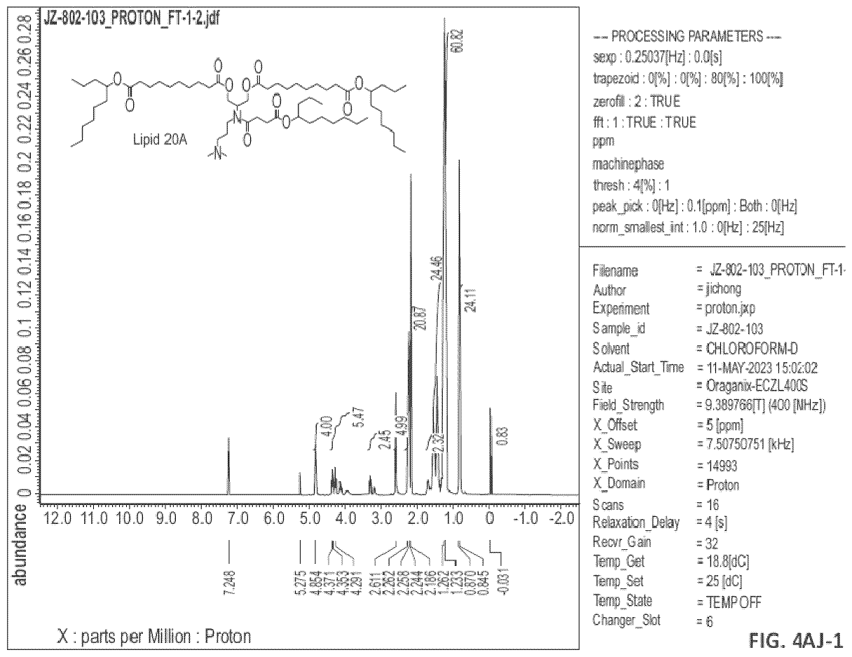

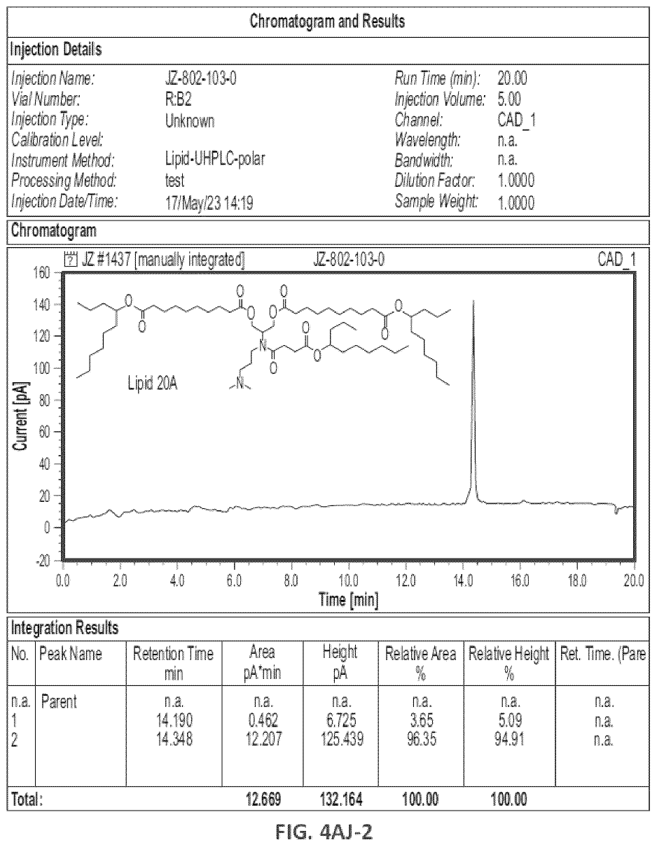

Lipid 1 was synthesized as provided in scheme 6 below and as follows. Starting material, 131-1 (0.75 mmol, 130 mg, 1.0 eq) was converted to the acid chloride (Step 1) using oxalyl chloride (3.7 mmol, 320 μl, 5 eq,) and DMF (10 μl, catalytic) in 6 mL of benzene. Product (143 mg, 98%) showed only one spot on TLC (as methyl ester) and was used without further purification for acylation (step 2) of intermediate 13-0. Intermediate 13-0 (0.35 mmol, 250 mg, 1.0 eq.) was acylated with crude acid chloride, 131-1 (0.75 mmol, 143 mg, 1.7 eq.) using TEA (240 μL, 5 eq, 1.8 mmol) and DMAP (10 mg, catalytic amount). Crude product was purified by column chromatography (2×) yielding 124 mg (76%) of pure Lipid 1 (>99% purity by LC-ELSD) and characterized by proton NMR and Mass Spectrometry (see FIG. 4A-1 for Lipid 1 NMR spectrum, and Table 5 for product mass).

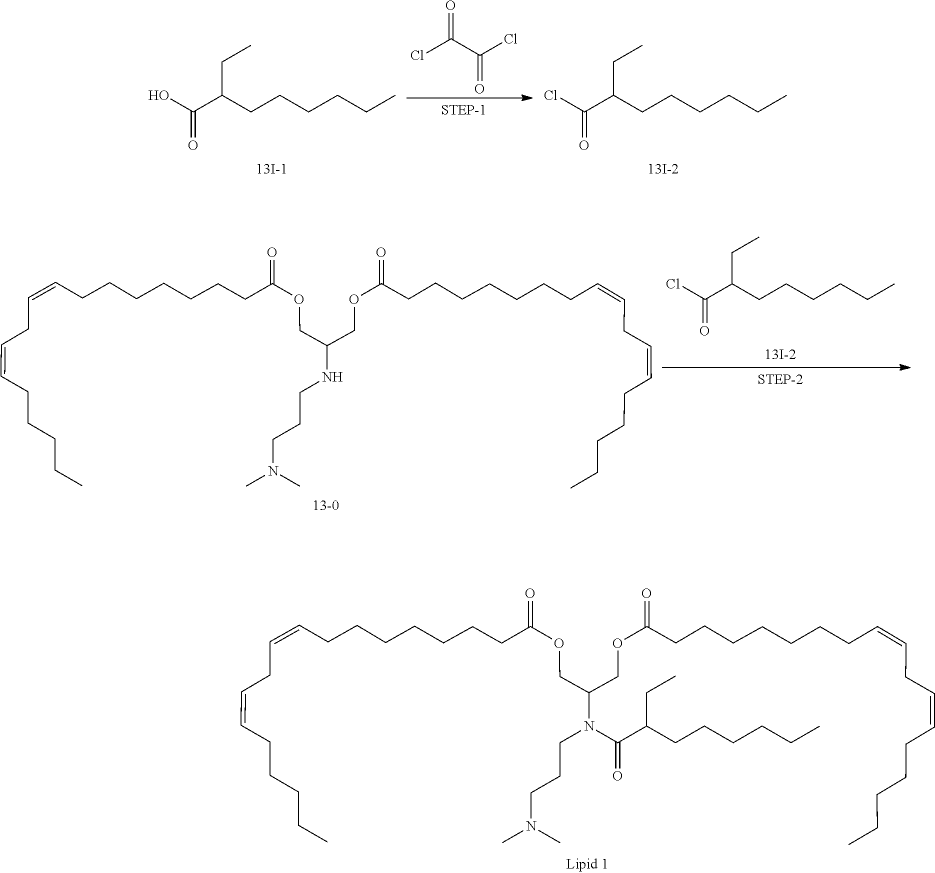 [SMILES: CCCCCCC(CC)C(=O)O ; O=C(Cl)C(=O)Cl ; CCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

### Synthesis of Lipid 3

Lipid 3 was synthesized as provided in scheme 7 below and as follows. Starting material, 13-13 (8.3 mmol, 1.30 g, 1.0 eq) was converted to the acid chloride, 13-13a (Step 1) using oxalyl chloride (2.8 mmol, 2.4 ml, 5 eq.) and DMF (100 μl, catalytic) in 60 mL of benzene. Product (1.44 g, 98%) showed only one spot on TLC (as methyl ester) and was used without further purification for acylation (step 2) of intermediate 13-0. Intermediate 13-0 (5.4 mmol, 3.78 g, 1.0 eq.) was acylated with crude acid chloride, 13-13a (1.44 g, 1.5 eq, 8.1 mmol) using TEA (3.76 mL, 5 eq, 27 mmol) and DMAP (50 mg, cat, catalytic amount) in benzene (100 mL). Crude product was purified by column chromatography (2×) yielding 2.1 g (46.3%) of pure Lipid 3 (≥99% purity by LC-ELSD) and characterized by proton NMR and Mass Spectrometry (see FIG. 4B-1 for Lipid 3 NMR spectrum, FIG. 4B-2 for Lipid 3 LC-MS, and Table 5 for product mass).

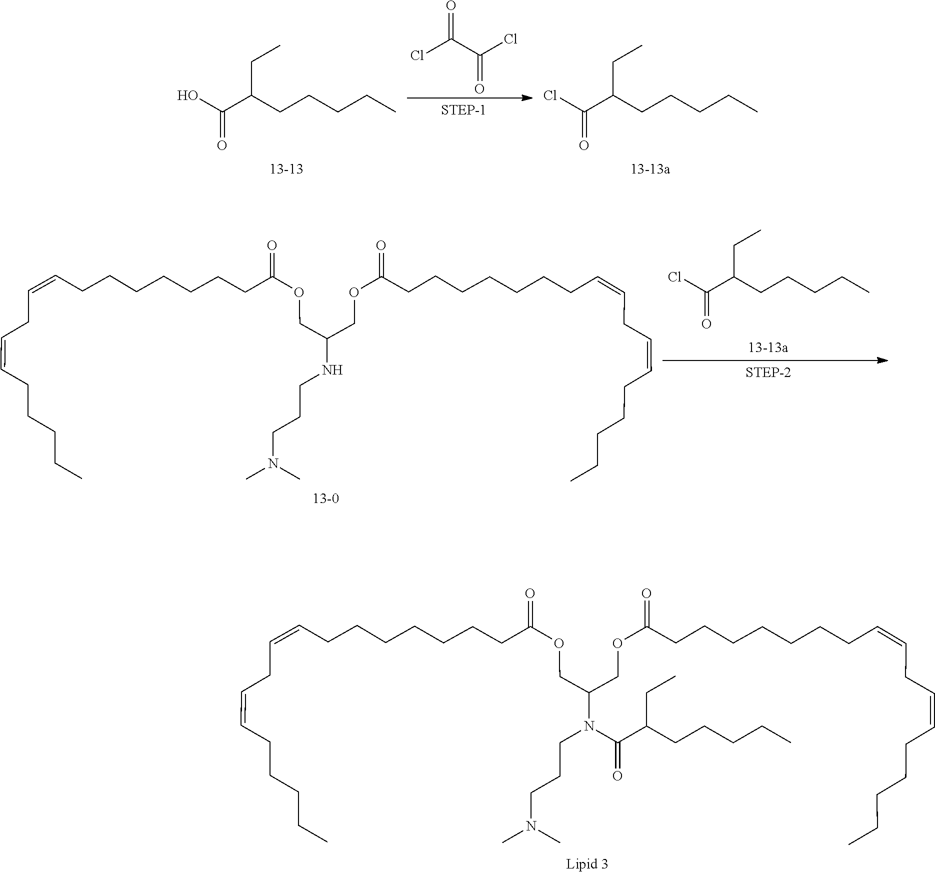 [SMILES: CCCCCC(CC)C(=O)O ; O=C(Cl)C(=O)Cl ; CCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

### Synthesis of Lipid 4

Lipid 4 was synthesized as provided in scheme 7 below and as follows. Starting material, 13-18 (0.95 mmol, 150 mg, 1 eq.) was converted to the acid chloride, 13-18′ (Step 1) using oxalyl chloride (3.23 mmol, 227 μl, 3.4 eq.) and DMF (10 μl, catalytic) in 6 mL of benzene. Product showed only one spot on TLC (as methyl ester) and was used without further purification for acylation (step 2) of intermediate 13-11b. Intermediate 13-11b (0.63 mmol, 444 mg, 1.0 eq.) was acylated with crude acid chloride, 13-18′ (167 mg, 1.5 eq, 0.95 mmol) using TEA (445 μL, 5.0 eq, 3.2 mmol) and DMAP (10 mg, catalytic amount) in benzene (10 mL). Crude product was purified by column chromatography (5×) yielding 140 mg (26%) of pure Lipid 4 (97% purity by LC-ELSD) and characterized by proton NMR and Mass Spectrometry (see FIG. 4C-1 for Lipid 4 NMR spectrum, FIG. 4C-2 for Lipid 4 LC-MS, and Table 5 for product mass).

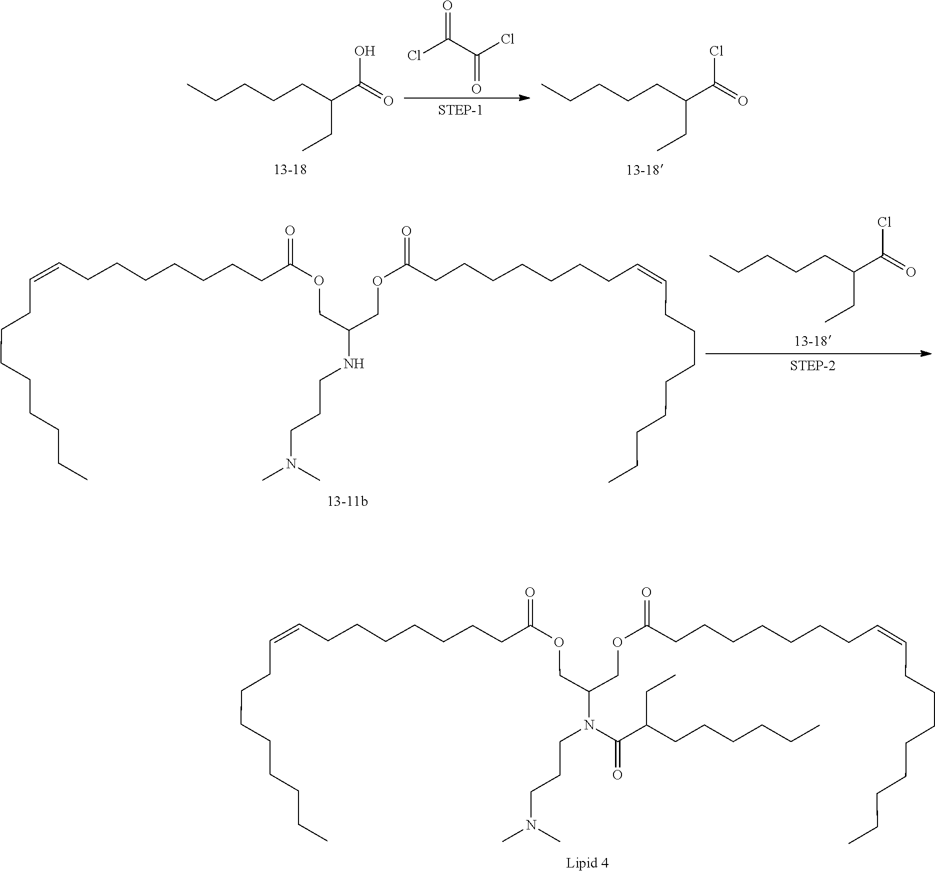 [SMILES: CCCCCC(CC)C(=O)O ; O=C(Cl)C(=O)Cl ; CCCCCC(CC)C(=O)Cl ; CCCCCCCC/C=C\CCCCCCCC(=O)OCC(COC(=O)CCCCCCC/C=C\CCCCCCCC)NCCCN(C)C ; CCCCCC(CC)C(=O)Cl ; CCCCCCCC/C=C\CCCCCCCC(=O)OCC(COC(=O)CCCCCCC/C=C\CCCCCCCC)N(CCCN(C)C)C(=O)C(CC)CCCCCC]

Synthesis of Lipid 5 and its(s) Isomer

The (S) isomer of lipid 5 was synthesized as provided in scheme 9-1 below and as follows. Starting material, Ethyl hexenoic acid 13m-1 (110 mg, 1.0 eq, 0.75 mmol) was converted to the acid chloride, 13m-2 (Step 1) using oxalyl chloride (320 μL, 1.0 eq, 3.7 mmol) and DMF (20 μl, catalytic) under reflux for 2 hours in 3 mL of benzene. Product showed only one spot on TLC (as methyl ester) and was used without further purification for acylation (step 2) of intermediate 13-0. Intermediate 13-0 (250 mg, 1.0 eq, 0.35 mmol) was acylated with crude acid chloride, 13m-2 (120 mg, 1.8 eq, 0.75 mmol) using TEA (240 μL, 5.0 eq, 1.8 mmol) and DMAP (10 mg, catalytic amount) in 10 mL benzene, overnight at room temperature. Crude product was purified by column chromatography (2×) yielding 95 mg (32%) of pure Lipid 5 (≥99% purity by LC-ELSD) and characterized by proton NMR and Mass Spectrometry (see FIG. 4D-1 for Lipid 5 NMR spectrum, FIG. 4D-2 for Lipid 5 LC-MS, and Table 5 for product mass).

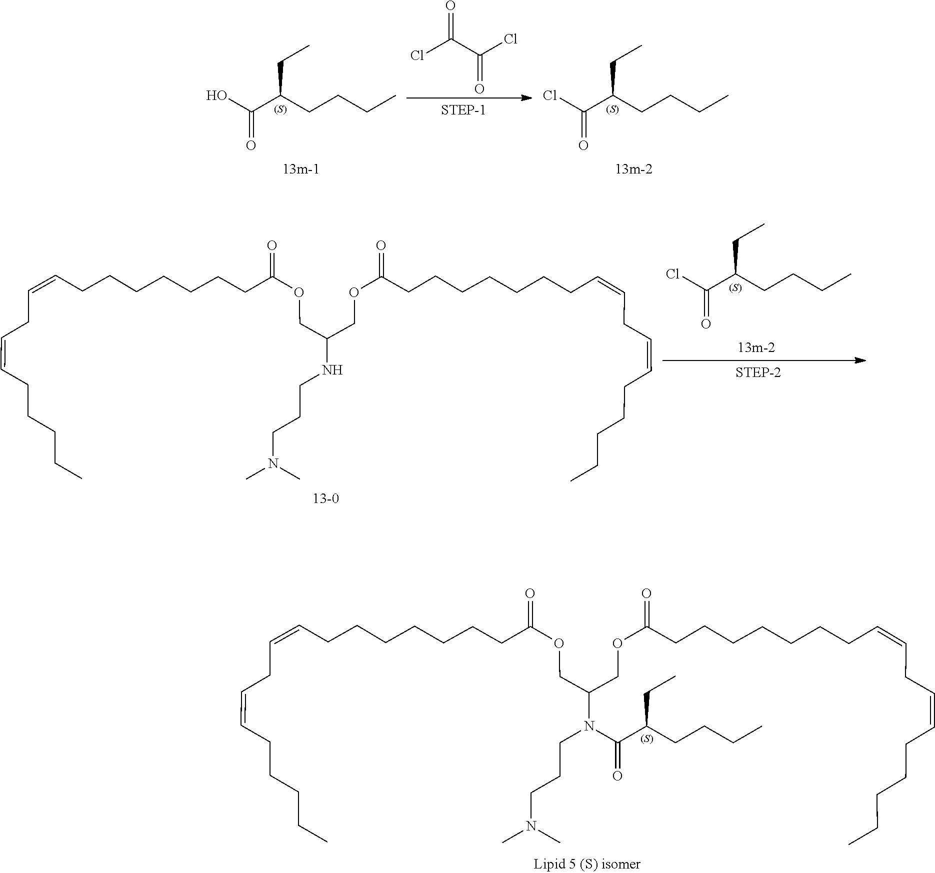

Lipid 5 as a racemic mixture was synthesized similarly as provided in scheme 9-2 below.

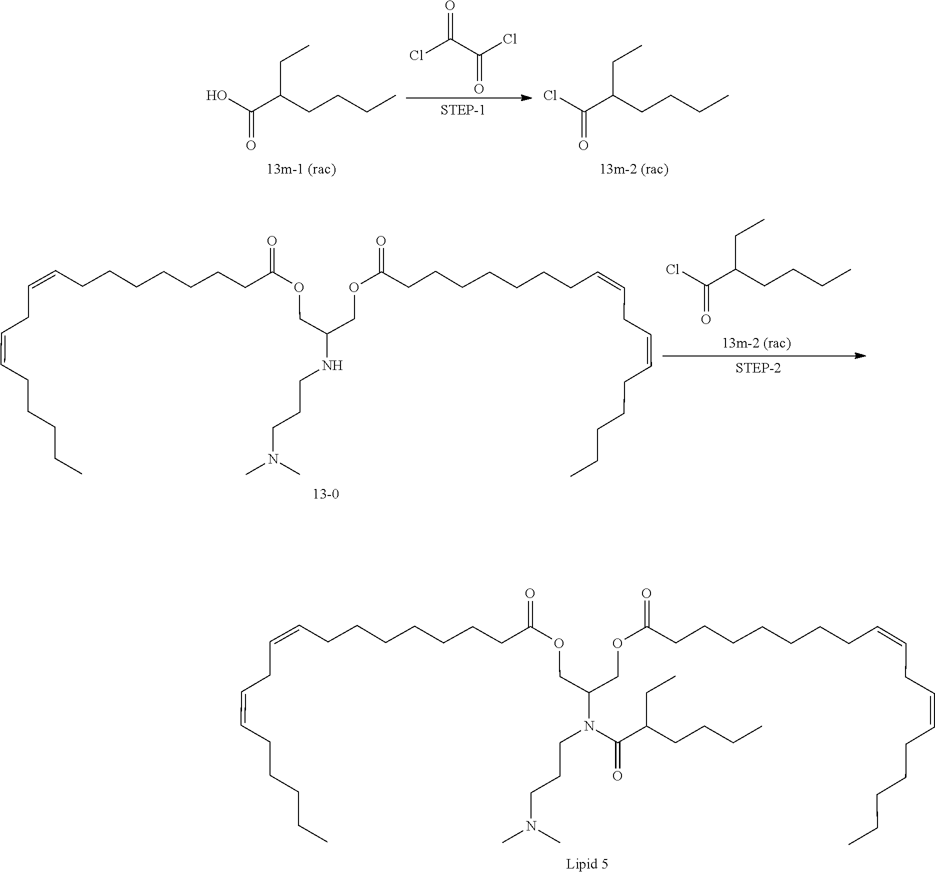

### Synthesis of Lipid 6

Lipid 6 was synthesized as provided in scheme 10 below and as follows. Starting material, 2-ethylnonanoic acid 13-14 (132 mg, 0.17 mmol, 1 eq.) was converted to the acid chloride, 13-14′ (Step 1) using oxalyl chloride (207 μl, 3.4 eq, 2.4 mmol) and DMF (10 μl, catalytic quantity) in 6 mL of benzene. Product showed only one spot on TLC (as methyl ester) and was used without further purification for acylation (step 2) of intermediate 13-0. Intermediate 13-0 (0.47 mmol, 330 mg, 1 eq.) was acylated with crude acid chloride, 13-14′ (145 mg, 1.5 eq, 0.7 mmol) using TEA (327 μL, 5.0 eq, 2.4 mmol) and DMAP (10 mg, catalytic amount) in 10 mL benzene. Crude product was purified by column chromatography (2×) yielding 75 mg (18%) of pure Lipid 6 (≥99% purity by LC-ELSD) and characterized by proton NMR and Mass Spectrometry (see FIG. 4E-1 for Lipid 6 NMR spectrum, FIG. 4E-2 for Lipid 6 LC-MS, and Table 5 for product mass).

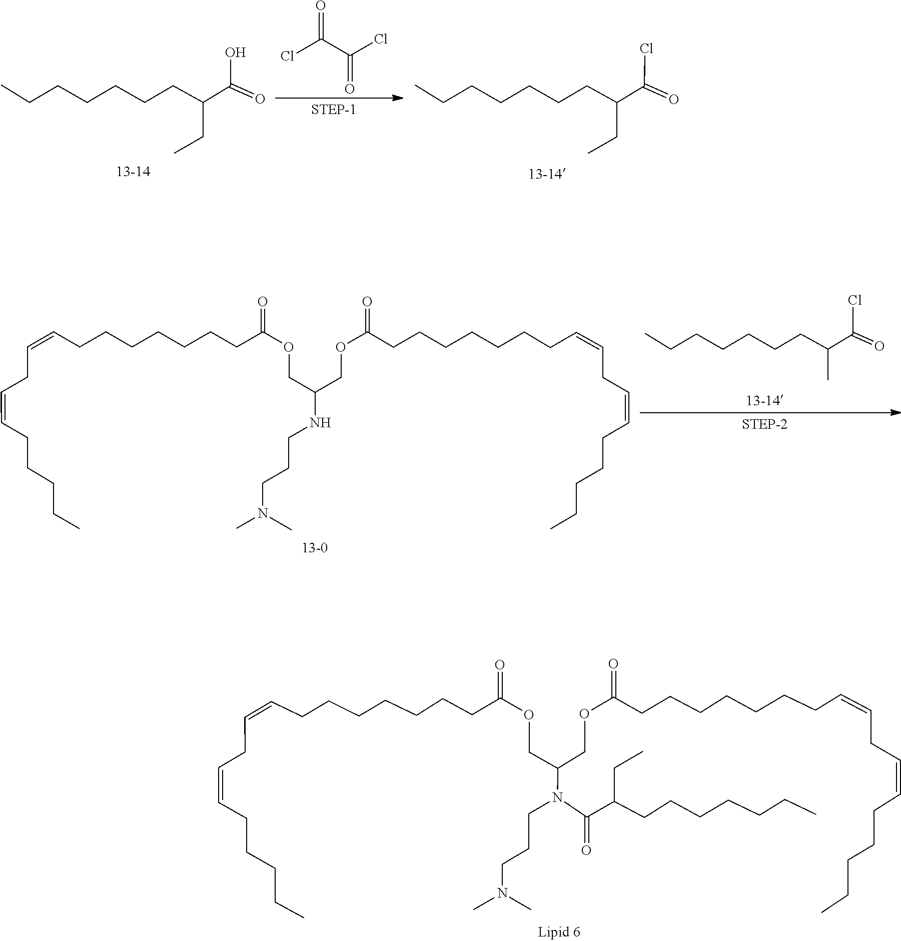

### Synthesis of Lipid 7

Lipid 7 was synthesized as provided in scheme 11 below and as follows. Starting material, heptanoic acid, 13-15 (23.1 mmol, 3.0 g, 1 eq.) was alkylated (step 1) with n-butyl bromide, 13-16 ((2.5 mL, 1.0 eq, 23.1 mmol) and 2.5 M n-butyl lithium in hexane (20.0 mL, 2.2 eq, 51 mmol) using diisopropylamine (7.2 mL, 2.2 eq, 51 mmol) in HMPA (4.4 mL) and 30 mL THF. 1.5 g (35%) of 2-butyl heptanoic acid, 13-17, was isolated from reaction mixture by flash chromatography. Intermediate 13-17 (360 mg, 0.94 mmol, 1 eq.) was converted to the acid chloride, 13-17′ (Step 2) using oxalyl chloride (6.6 mmol, 568 μl, 3.4 eq.) and DMF (5 μl, catalytic) in 3 mL of benzene. Product showed only one spot on TLC (as methyl ester) and was used without further purification for acylation (step 3) of intermediate 13-0. Intermediate 13-0 (0.64 mmol, 450 mg, 1 eq.) was acylated with crude acid chloride, 13-17′ (395 mg, 3.0 eq, 1.94 mmol), TEA (446 μL, 5.0 eq, 3.2 mmol), DMAP (10 mg) in 10 mL of benzene. Crude product was purified by column chromatography (2×) yielding 228 mg (41%) of pure Lipid 7 (≥99% purity by LC-ELSD) and characterized by proton NMR and Mass Spectrometry (see FIG. 4F-1 for Lipid 7 NMR spectrum, FIG. 4F-2 for Lipid 7 LC-MS, and Table 5 for product mass).

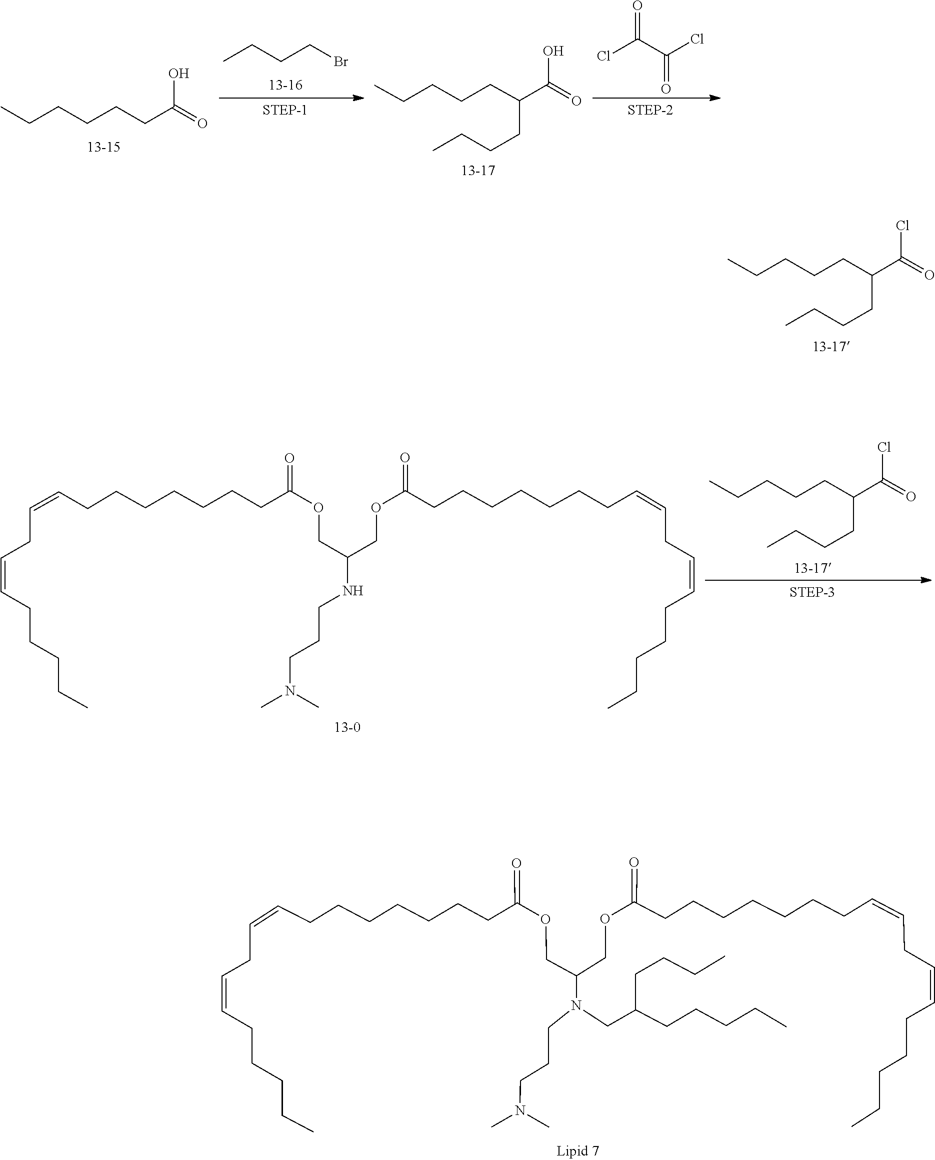

### Synthesis of Lipids 2, 8, 9 and 10 by N-Acylation of Intermediate 13-0 Using Carbodiimide Activation of the Corresponding Carboxylic Acids

### Synthesis of Lipid 2

Lipid 2 was synthesized as provided in scheme 12 below and as follows. Intermediate 13-0 (0.14 mmol, 320 mg, 1.0 eq.) was acylated with nonanoic acid 13-12 (1.15 mmol, 198 μL, 2.5 eq.), EDCI (1.15 mmol, 221 mg, 2.5 eq.), DIPEA (1.15 mmol, 198 μL, 2.5 eq.), and DMAP (0.05 mmol, 6.4 mg, 0.1 eq.) in 5 mL DCM. Crude product was purified by column chromatography (3×) yielding 107 mg (%) of pure Lipid 2 (≥99% purity by LC-ELSD) and characterized by proton NMR and Mass Spectrometry (see FIG. 4G-1 for Lipid 2 NMR spectrum, FIG. 4G-2 for Lipid 2 LC-MS, and Table 5 for product mass).

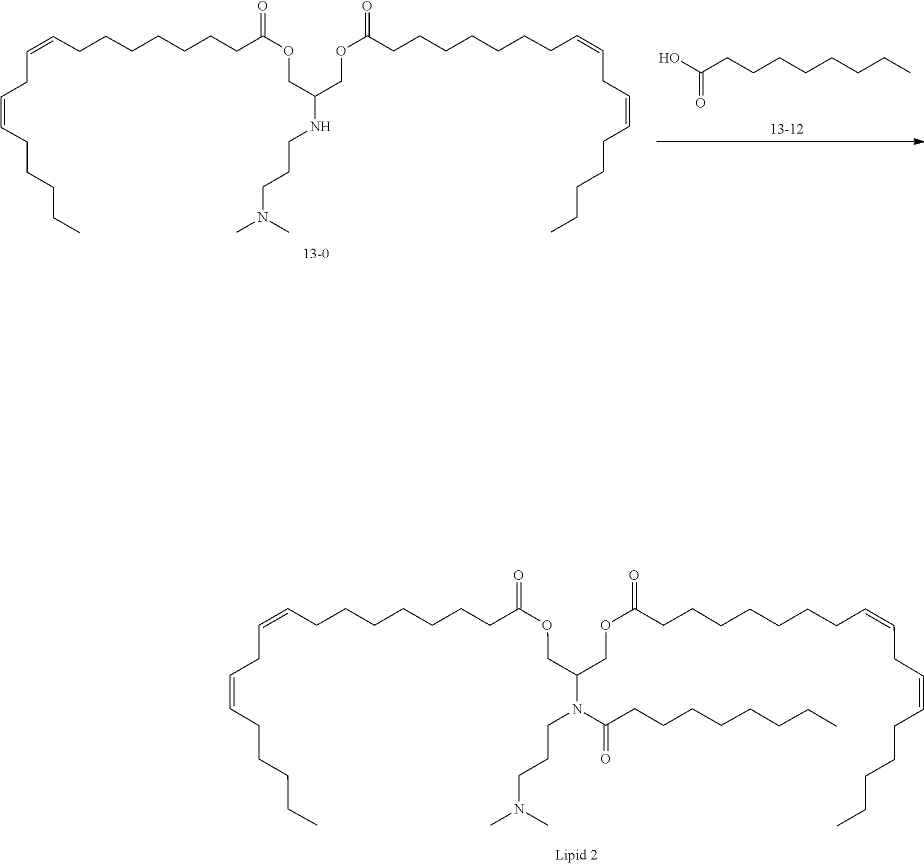 [SMILES: CCCCC/C=C\C/C=C\CCCCCCCC(=O)OCC(COC(=O)CCCCCCC/C=C\C/C=C\CCCCC)NCCCN(C)C ; CCCCCCCCC(=O)O ; CCCCC/C=C\C/C=C\CCCCCCCC(=O)OCC(COC(=O)CCCCCCC/C=C\C/C=C\CCCCC)N(CCCN(C)C)C(=O)CCCCCCCC]

### Synthesis of Lipid 8

Lipid 8 was synthesized as provided in scheme 13 below and as follows. Alkene, 13-48 was accessed via the HWE reaction (step 1) of octan-3-one, 13-46 (2 g, 15.6 mmol) with ethyl 2-(diethoxyphosphoryl)acetate, 13-47 (7.0 g, 2.0 eq, 31.2 mmol), 2M NaHMDS in THF (15.6 mL, 2.0 eq, 31.2 mmol), and 9 ml THF solvent. Reaction workup yielded 2.38 g (77%) of 13-48 confirmed by NMR, product mass and single TLC spot. Alkene, 13-48 (5.1 mmol, 1 g, 1 eq.) was hydrogenated (step 2) using Pd/C (50 mg) in 8 mL ethyl acetate yielding intermediate 13-48 (958 mg, 77%). Ester hydrolysis (step 3) of 13-49 (5.1 mmol, 412 mg) using THF/MeOH/1M LiOH (3.0/2.0/3.0 mL) yielded carboxylic acid intermediate 13-50 (336 mg, 95%). Intermediate 13-0 (0.33 mmol, 234 mg) was acylated with 13-50 (0.66 mmol, 115 mg, 2.0 eq.) using EDCI (0.66 mmol, 102 mg, 2.0 eq.), DIPEA (0.66 mmol, 114 μL, 2.0 eq.), DMAP (0.33 mmol, 41 mg, 1.0 eq.), in 2 mL DCM yielding 77 mg (27%) of pure Lipid 8 (≥99% purity by LC-ELSD) and characterized by proton NMR and Mass Spectrometry (see FIG. 4H-1 for Lipid 8 NMR spectrum, FIG. 4H-2 for Lipid 8 LC-MS, and Table 5 for product mass).

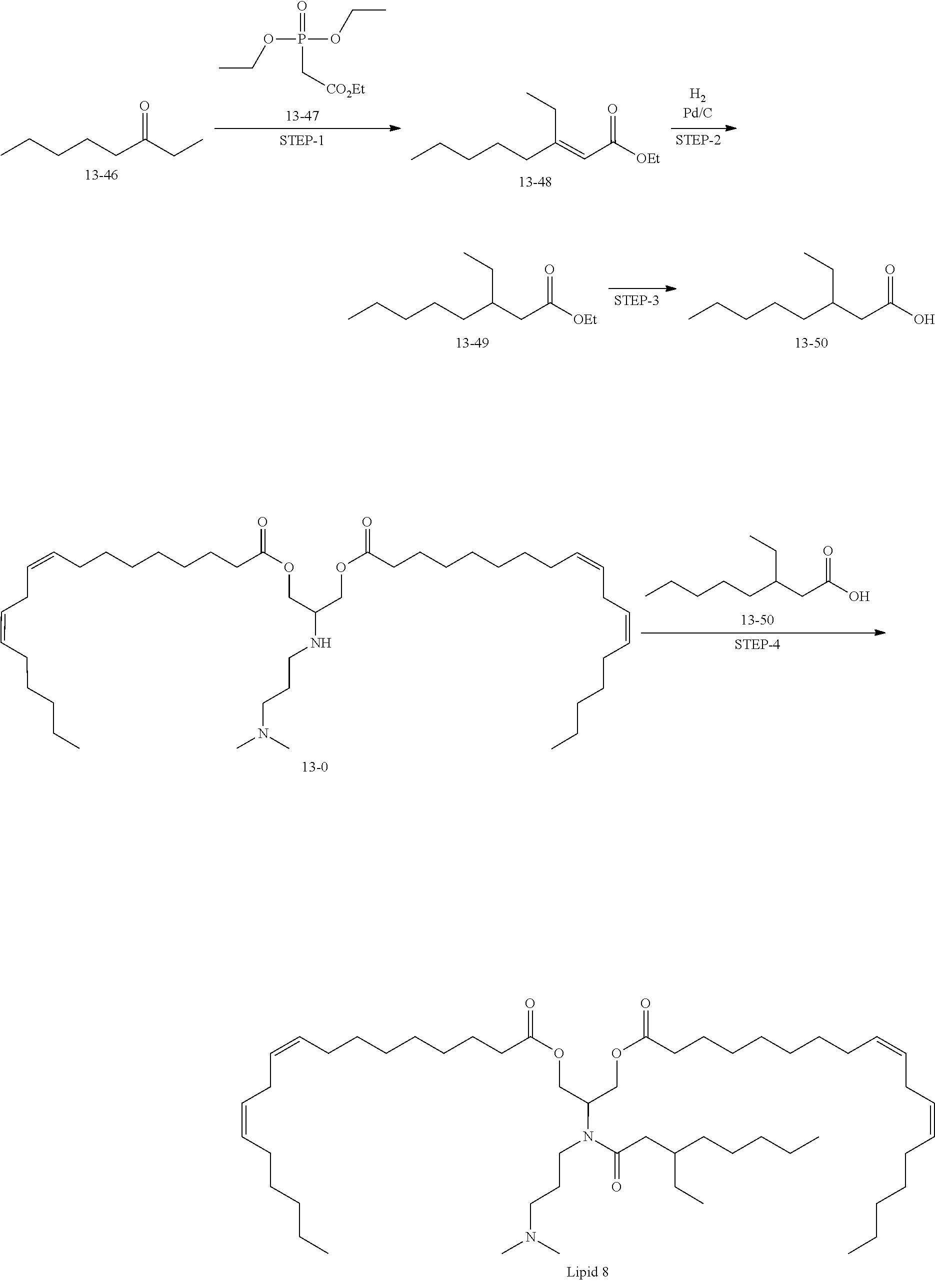

### Synthesis of Lipid 9

Lipid 9 was synthesized as provided in scheme 14 below and as follows. Starting material, decan-4-ol, 13-29 (32.0 mmol, 5.0 g, 1.0 eq.) was acylated with succinic acid, 13-30 (6.3 g, 2.0 eq, 63.0) using DMAP (3.55 g, 1.0 eq, 32.0 mmol) and pyridine (5.0 ml) in 5 mL THF. Crude product was purified by column chromatography (1×) to obtain 4.26 g (81%) of pure acid intermediate 13-31. Intermediate 13-0 (2.1 mmol, 1.5 g, 1 eq.) was acylated with 13-31 (2.13 mmol, 0.554 g, 1.1 eq), using DIPEA (745 μL, 4.26 mmol, 2.5 eq), EDCI (820 mg, 4.26 mmol, 2.5 eq), and DMAP (480 mg, 0.43 mmol, 0.25 eq), in 50 mL DCM. Crude product was purified by column chromatography (3×) yielding 1.4 g (73%) of pure Lipid 9 (≥99% purity by LC-ELSD) and characterized by proton NMR and Mass Spectrometry (see FIG. 4I-1 for Lipid 9 NMR spectrum, FIG. 4I-2 for Lipid 9 LC-MS, and Table 5 for product mass).

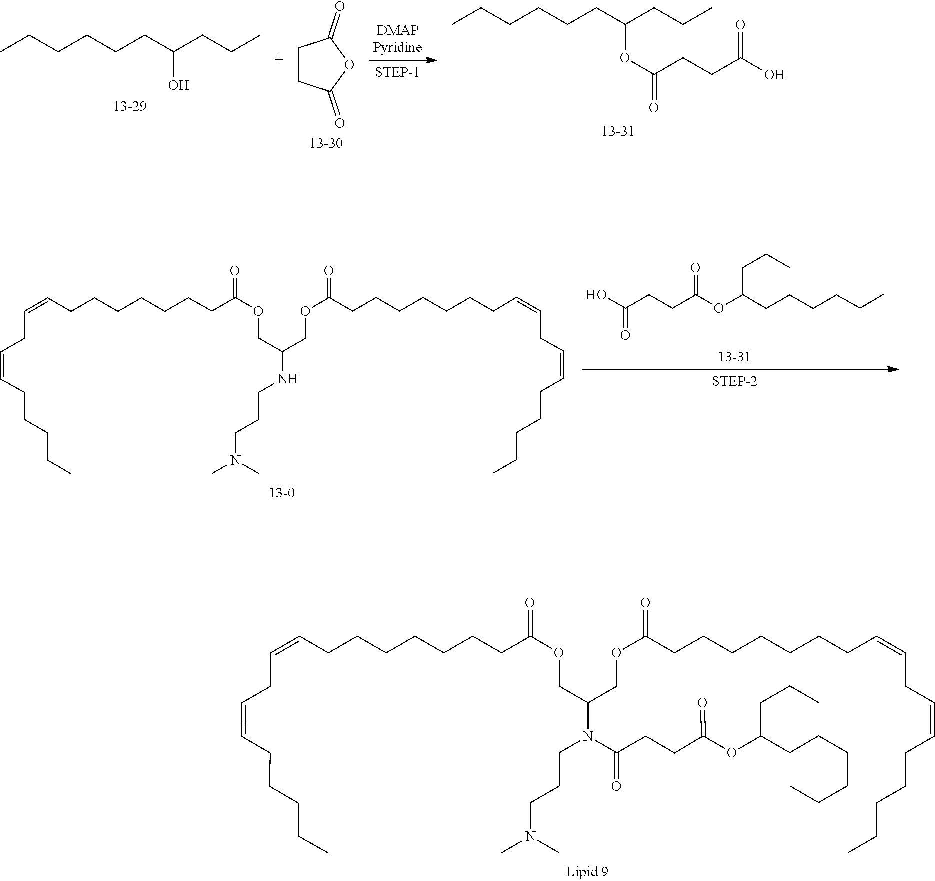

### Synthesis of Lipid 10 and its (S) Isomer

The (S) isomer of lipid 10 was synthesized as provided in scheme 15-1 below and as follows. Starting material, Octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM. Crude product was purified by column chromatography (1×) to obtain 1.1 g (31%) of pure acid intermediate 13-47. Intermediate 13-0 (250 mg, 1.0 eq, 0.36 mmol) was acylated with 13-47 (123 mg, 1.5 eq, 0.53 mmol), using EDCI (207 mg, 3.0 eq, 1.80 mmol), DIPEA (188 μL, 3.0 eq, 1.8 mmol) and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

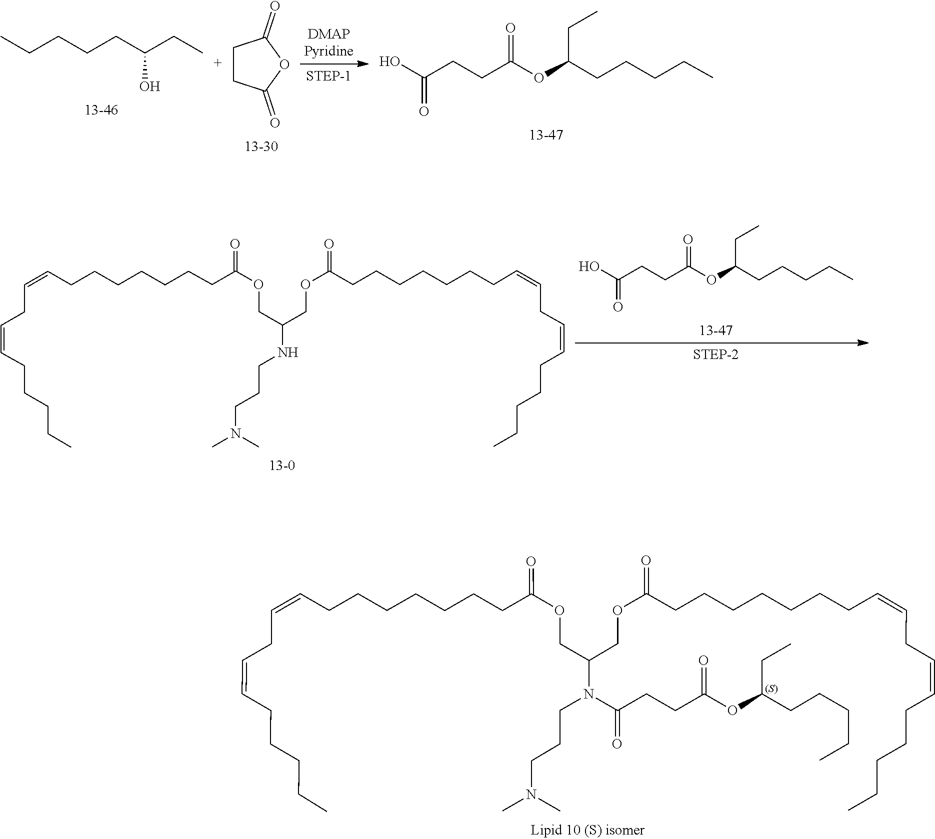

Lipid 10 as a racemic mixture was synthesized similarly as provided in scheme 15-2 below. Starting material, octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM to obtain intermediate 13-47. Crude product was purified by column chromatography (1×) to obtain 1.1 g (31%) of pure acid intermediate 13-47. 13-0 (250 mg, 1.0 eq, 0.36 mmol) was acylated with 13-38 (123 mg, 1.5 eq, 0.53 mmol) using DIPEA (188 μL, 3.0 eq, 1.8 mmol), EDCI (207 mg, 3.0 eq, 1.80 mmol), and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 4J-1 for Lipid 10 NMR spectrum, FIG. 4J-2 for Lipid 10 LC-MS, and Table 5 for product mass).

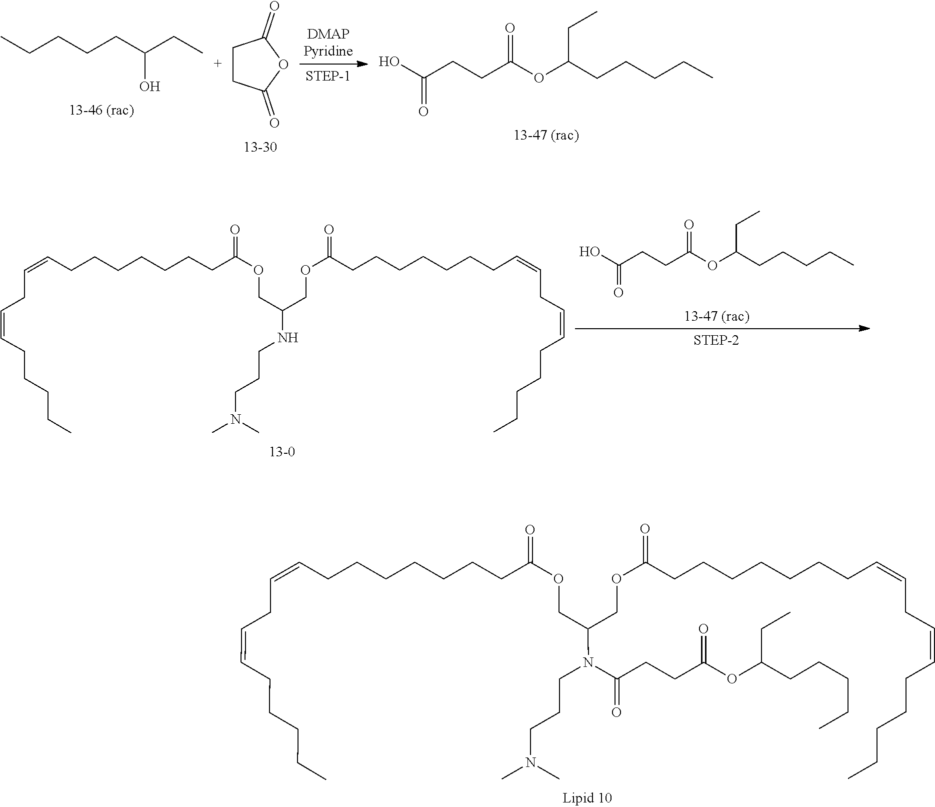

### Synthesis of Lipid 11 by N-Acylation of Intermediate 13-0 Using the Corresponding Acid Chloride

Synthesis of Lipid 11 and its(s) Isomer

The (S) isomer of lipid 5 was synthesized as provided in scheme 16-1 below and as follows. Starting material, benzyl alcohol, 13-39′ (18.5 mmol, 2 g) was used to acylate compound 13-39 (4.8 g, 1.5 eq, 27.8 mmol) using EDCI (5.4 g, 1.5 eq, 27.8 mmol), DIPEA (4.6 mL, 1.5 eq, 27.8 mmol), and DMAP (463 mg, 0.2 eq, 3.7 mmol) yielding 3.6 g (74%) of column purified intermediate 13-40 (product confirmed by mass spectrometry and proton NMR). Intermediate, 13-40 (684 mg, 2.6 mmol, 1 eq.) was deprotected in acetic acid to obtain intermediate, 13-41 (~600 mg, quantitative and product structure was confirmed by mass spectrometry and proton NMR). Additional quantity of intermediate 13-41 was generated and 1.68 g, 7.5 mmol of 13-41 was selectively protected at the hydroxyl group using TBSCl (1.7 g, 11.25 mmol, 1.5 eq), TEA (5.3 mL, 5.0 eq, 37.5 mmol), and DMAP (92 mg, 0.75 mmol, 0.1 eq), in 20 mL DCM yielding protected intermediate 13-41a (~2.5 g, quantitative) (product mass was confirmed by mass spectrometry and proton NMR). Intermediate 13-41a (1.61 g, 4.76 mmol) was esterified with n-hexyl alcohol 13-34 (2.94 mL, 23.8 mmol, 5.0 eq) using EDCI (2.76 g, 14.2 mmol, 3.0 eq), DIPEA (1.6 mL, 2.0 eq, 9.52 mmol), and DMAP (580 mg, 4.76 mmol, 1.0 eq) in 11.0 mL of DCM to obtain 13-41b (0.95 g, 48%). Additional quantity of 13-41b was generated and total of 1.36 g (3.2 mmol) was deprotected using HF-pyridine (5.8 mL, 80.6 mmol, 25 eq.) in 30 mL THF to obtain intermediate 13-41c (837 mg, 84%). Intermediate 13-41c (456 mg, 1.48 mmol) was acylated with n-butanoyl chloride, 13-42 (760 μL, 7.4 mmol, 5.0 eq) in 4.0 mL pyridine (4.0 mL) yielding compound 13-44 (505 mg, 90%). Intermediate 13-44 (505 mg, 1.34 mmol) was deprotected using Pd/C (30 mg) in 3.0 mL ethyl acetate yielding compound 13-45 (370 mg, 96%). Compound 13-45 (188 mg, 0.65 mmol) was converted to the acid chloride intermediate using oxalyl chloride (190 μg, 3.4 eq, 2.2 mmol) and DMF (10 μL, catalytic quantity), in 3 mL Benzene. Product showed only one spot on TLC (as methyl ester) and was used without further purification for acylation (step 9) of intermediate 13-0. Intermediate 13-0 (152 mg, 0.22 mmol, 1 eq.) was acylated with crude acid chloride, 13-45′ (200 mg, 3.0 eq, 0.65 mmol), TEA (152 μL, 5.0 eq, 1.1 mmol), DMAP (10 mg) in 5 mL of benzene to obtain Lipid 11. Crude product was purified by column chromatography to yield 77 mg (37%) of pure Lipid 11 (≥99% purity by LC-ELSD) and characterized by proton NMR and Mass Spectrometry (see FIG. 4K-1 for Lipid 11 NMR spectrum, FIG. 4K-2 for Lipid 11 LC-MS, and Table 5 for product mass).

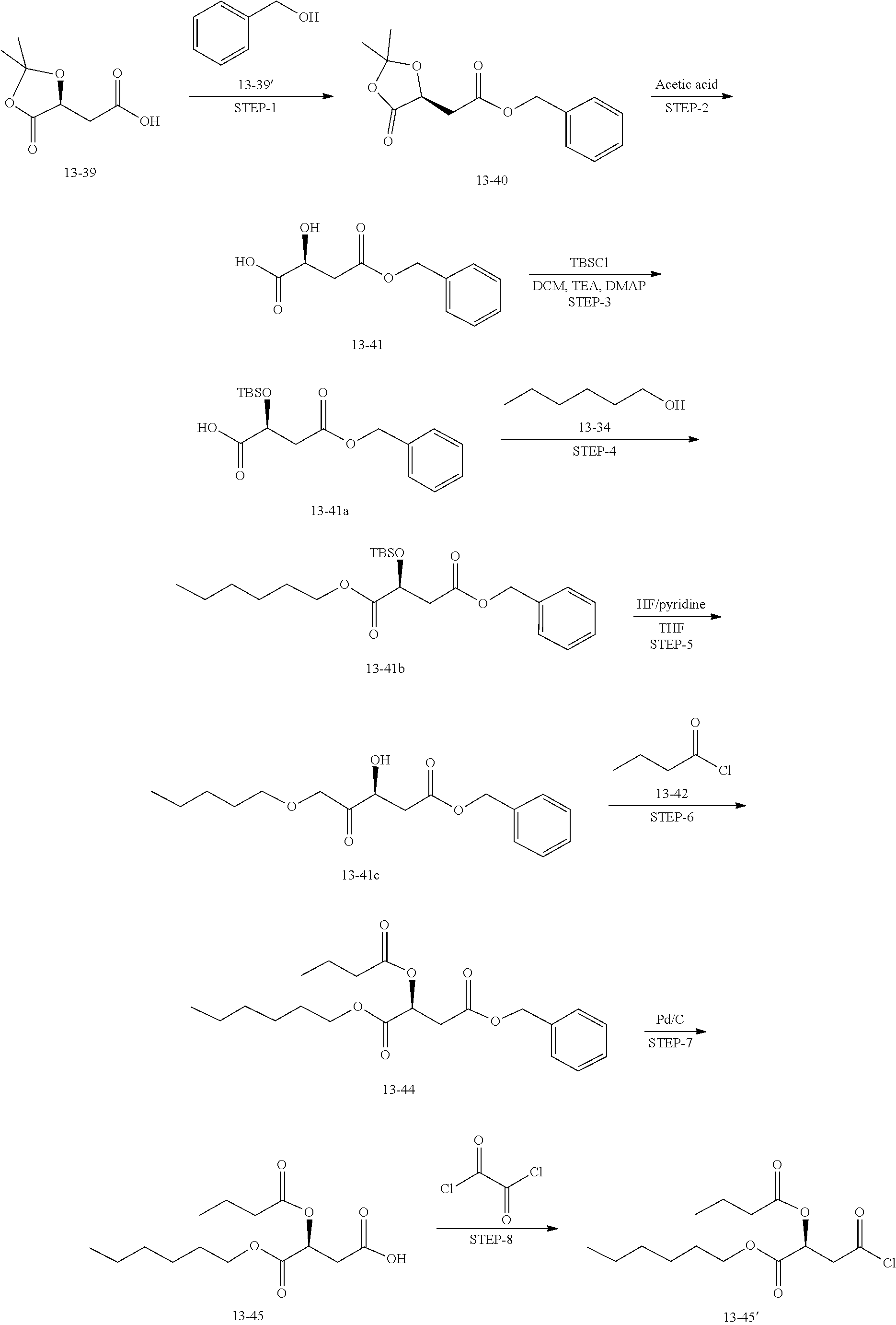

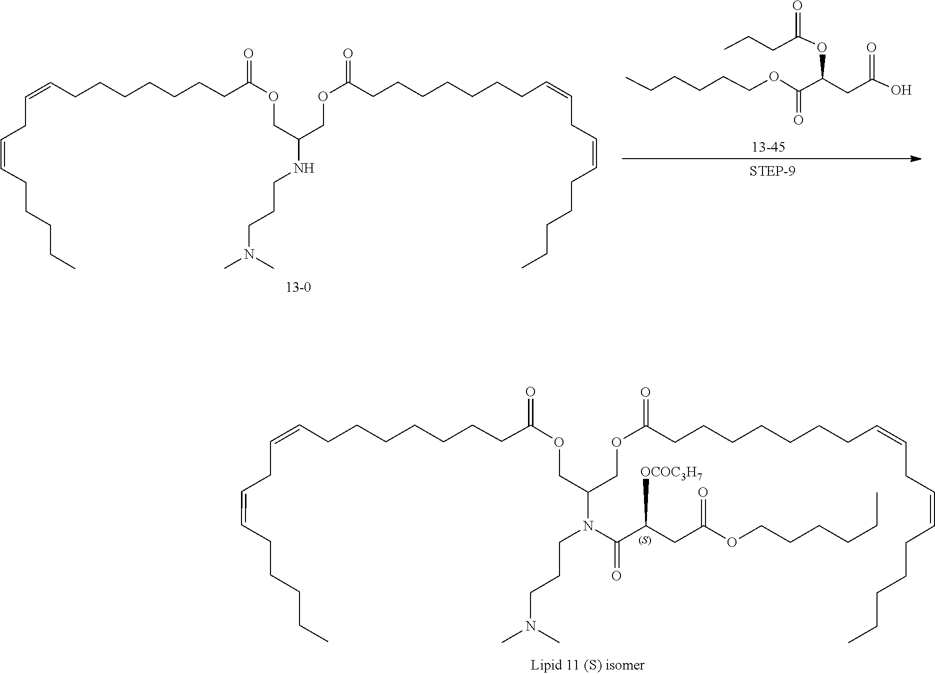

Lipid 11 as a racemic mixture was synthesized similarly as provided in scheme 16-2 below.

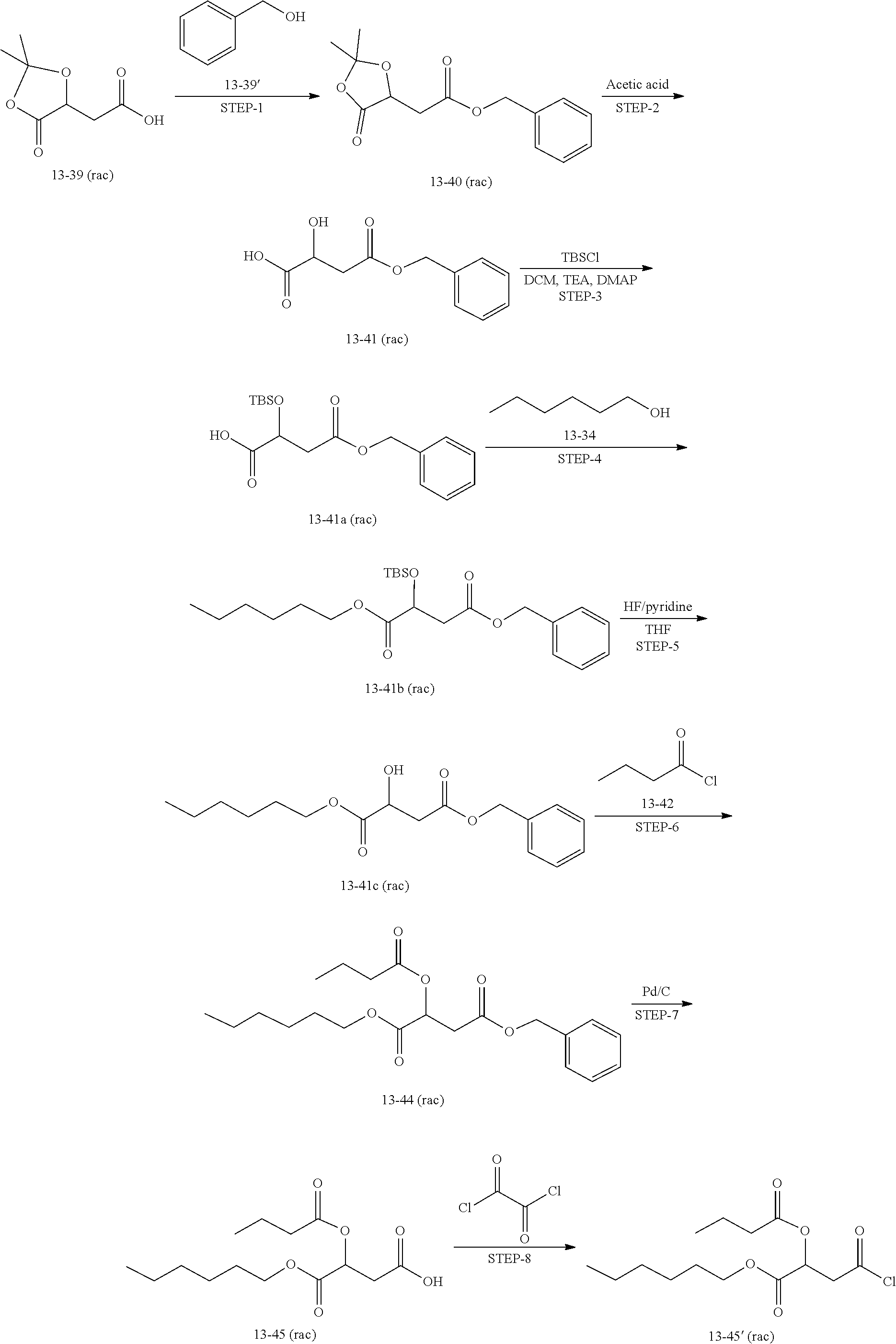

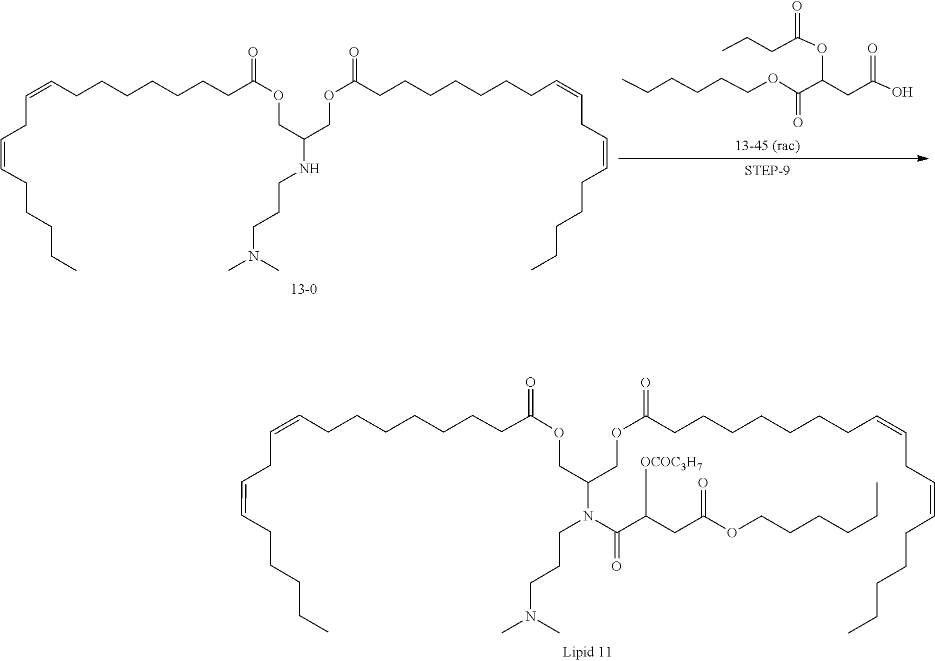

### Synthesis of Lipid 12

Lipid 12 was synthesized as provided in scheme 34 below and as follows. Starting material, 14-3 (3 g, 1.0 eq, 22.37 mmol) was selectively protected in trifluoroacetic anhydride (11.27 g, 2.4 eq, 53.69 mmol) and Benzyl alcohol (15 mL) at room temperature, overnight yielding intermediate 14-4. Crude product was purified by column chromatography (1×) to obtain 4.7 g (96%) purified 14-4. Subsequent acylation of 14-4 (1.0 eq, 4.44 mmol) with n-butanol, 13-34 (4.55 g, 10.0 eq, 44.60 mmol) using EDCI (1.71 g, 2 eq, 8.92 mmol) and DMAP (1.089 g, 2 eq, 8.92 mmol) in 10 mL DCM at RT, overnight yielded 14-5. Crude product was purified by column chromatography (1×) to obtain 800 mg (58%) purified 14-5. Acylation of the free hydroxyl of 14-5 (800 mg, 1.0 eq, 2.59 mmol) with hexanoyl chloride (1.39 g, 4.0 eq, 10.37 mmol) using TEA (1.31 g, 5 eq, 12.97 mmol) and DMAP (10 mg, catalytic amount) in 10 mL toluene at room temperature, overnight yielded intermediate 14-7. Purification of crude product by column chromatography (1×) yielded 470 mg (46%) purified 14-7. Intermediate 14-7 (470 mg, 1 eq., 3.4 mmol) yielded 340 mg (93%) of free acid 14-8. Crude 14-8 (56 mg, 1 eq., 0.18 mmol) was converted to the corresponding chloride, 14-8′, using Oxalyl Chloride (50 μL, 3.4 eq, 0.60 mmol) and DMF (0.2 μL, Catalytic amount) in 1 mL Toluene at room temperature, overnight to afford 56 mg of crude chloride 14-8′. N-acylation of 13-0 (42 mg, 1 eq., 0.059 mmol) with 14-8′ (56.0 mg, 3.0 eq, 0.17 mmol) using TEA (39.0 μL, 5.0 eq, 0.29 mmol) and DMAP (10 mg, Catalytic amount) in 3 mL Toluene yielded Lipid 12. Crude product was purified by column chromatography (1×) to obtain pure Lipid 12 (23 mg, 39%) (≥99% purity by LC-ELSD) and characterized by proton NMR and Mass Spectrometry (see FIG. 4L-1 for Lipid 12 NMR spectrum, FIG. 4L-2 for Lipid 12 LC-ELSD chromatogram, and Table 5 for product mass).

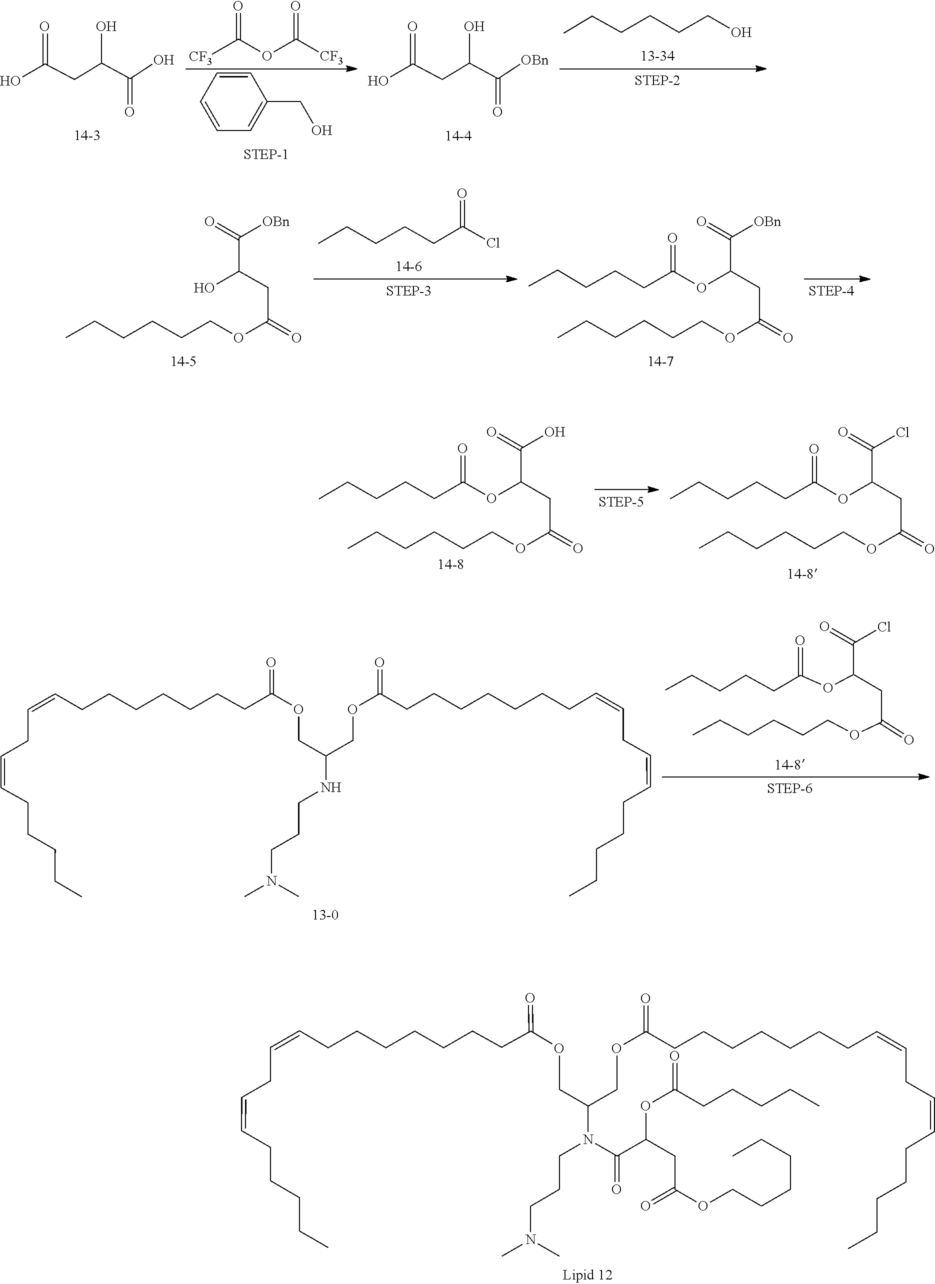

### Synthesis of Lipids 13 by N-Acylation of Intermediate 13-0 Carbodiimide Activation of the Corresponding Carboxylic Acid

### Synthesis of Lipid 13

Lipid 13 was synthesized as provided in scheme 17 below and as follows. Starting material 13-32 (4.8 g, 2.0 eq, 25.0 mmol) was esterified with 1-Butanol (1.13 mL, 1 eq, 12.4 mmol) using EDCI (4.8 g, 2 eq, 25.0 mmol), DIPEA (4.35 mL, 2 eq, 25.0 mmol), and DMAP (280 mg, 0.2 eq, 2.5 mmol) in 20 mL DCM to obtain intermediate 13-33. Crude product was purified by column chromatography to obtain 2.78 g (44%) of pure intermediate 13-33. Intermediate 13-36 was accessed by acylation of n-hexanol (2 g, 1.0 eq, 19.6 mmol) with 2-bromoacetyl bromide, 13-35 (5.05 g, 1.3 eq, 25.0 mmol) using NaHCO₃ (3.95 g, 2.4 eq, 47.0 mmol) in 50 mL acetonitrile. Crude product was purified by column chromatography (1×) to obtain 4.32 g (97%) of pure intermediate 13-36.

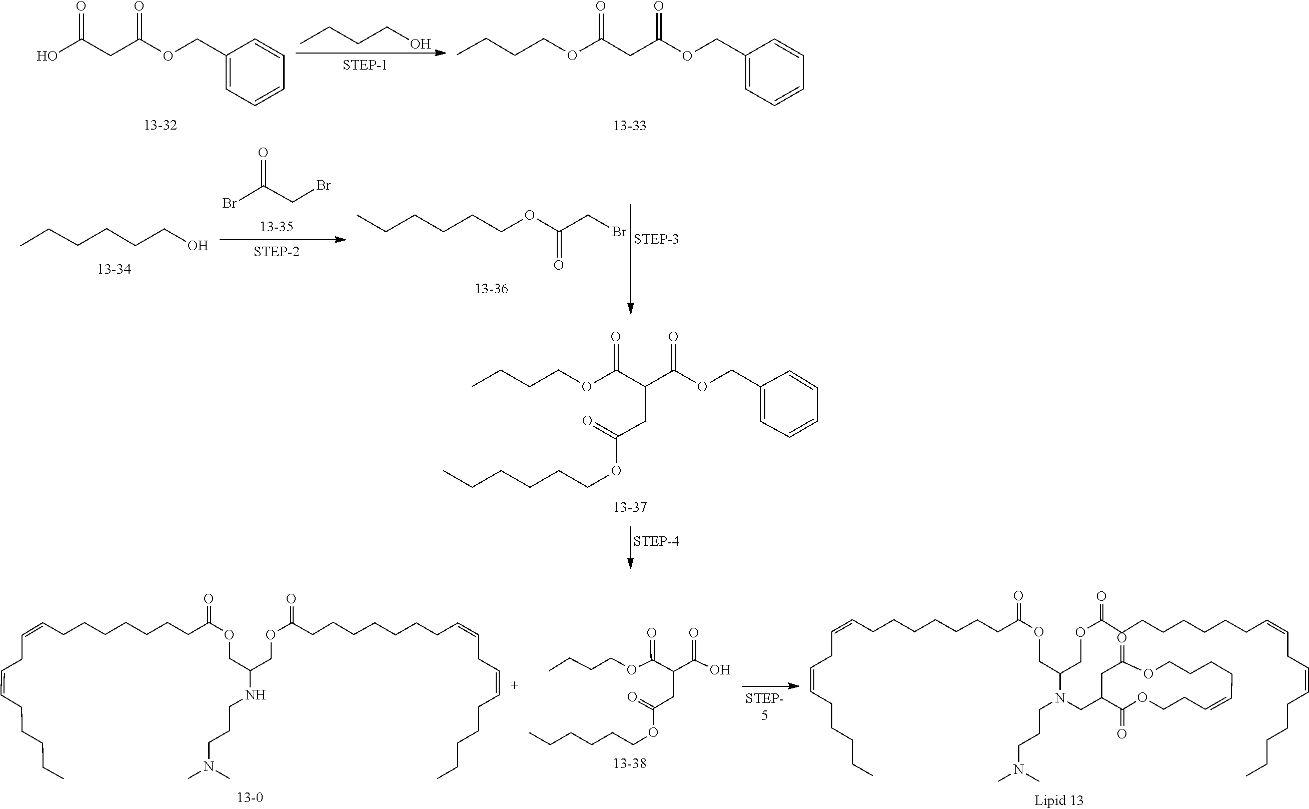

Intermediate 13-37 was accessed by in situ generation of the nucleophilic carbanion of 13-33 (1.25 g, 1.0 eq., 5.0 mmol) using NaH (200 mg, 1.0 eq, 5.0 mmol) in 8 mL DMF and displacement reaction with intermediate 13-36 (1.1 g, 1.0 eq, 5.0 mmol). Crude product was purified by column chromatography (2×) to 1.15 g (58%) of pure intermediate 13-37. Free acid intermediate 13-38 was obtained by deprotection (Pd/C, 230 mg catalyst and hydrogen gas in methanol) of intermediate 13-37 (1.15 g, 1.0 eq, 2.9 mmol). Crude product was purified by column chromatography (4×) to obtain 88 mg (9%) of pure intermediate 13-38. Intermediate 13-0 (105 mg, 1.0 eq, 0.04 mmol) was acylated with 13-38 (2.13 mmol, 0.554 g, 1.1 eq), using DIPEA (78 μL, 3.0 eq, 0.45 mmol), EDCI (87 mg, 3.0 eq, 0.45 mmol), and DMAP (5 mg, 0.3 eq, 0.04 mmol), in 2 mL DCM. Crude product was purified by column chromatography (3×) yielding 41 mg (27%) of pure Lipid 13 (≥99% purity by LC-ELSD) and characterized by proton NMR and Mass Spectrometry (see FIG. 4M-1 for Lipid 13 NMR spectrum, FIG. 4M-2 for Lipid 13 LC-MS, and Table 5 for product mass).

### Synthesis of Lipid 14A by N-Acylation of Intermediate 14-11 Using the Corresponding Acid Chloride

### Synthesis of Lipid 14A

Lipid 14A was synthesized as provided in Scheme 37 below and as follows.

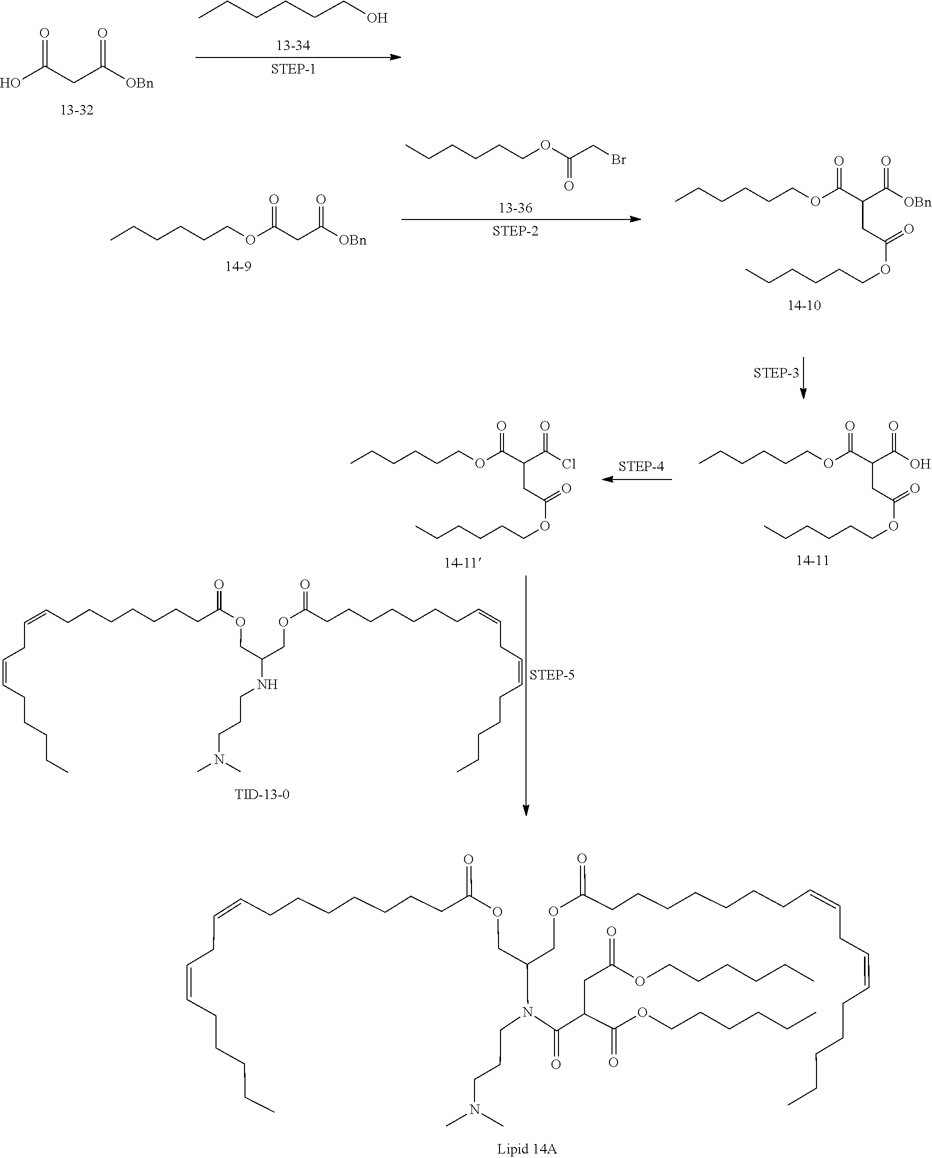

Starting material, mono-benzyl protected malonic acid, 13-32 (9.26 mmol, 1.8 g, 1.0 eq.) was esterified with N-hexanol, 13-34 (92.69 mmol, 9.47 g, 10.0 eq.) using EDCI (18.53 mmol, 3.55 g, 2 eq.) and DMAP (2.26 g, 2 eq, 18.53 mmol) in DCM (20 mL) at room temperature, overnight to obtain intermediate, 14-9 (2.04 g, 79%), Compound 13-36 was prepared by reacting bromoacetyl bromide (38.17 mmol, 7.70 g, 1.3 eq.) with 3.0 g of N-hexanol, 13-34 (1.0 eq, 29.36 mmol) using NaHCO₃ (5.9 g, 2.4 eq, 70.47 mmol) in 30 mL of acetonitrile (0° C. to room temperature) overnight to obtain 6.0 g (91.6%) of intermediate 13-36. Intermediate 14-9 (7.2 mmol, 2.03 g, 1.0 eq.) was converted to the corresponding carbanion and reacted with bromoacetyl bromide (7.70 g, 1.3 eq, 38.17 mmol) using NaHCO₃ (5.9 g, 2.4 eq, 70.47 mmol) in 30 mL of acetonitrile (0° C. to room temperature, overnight) to obtain Intermediate, 14-10 (1.15 g, 38%).

Deprotection of Intermediate 14-10 (3.4 mmol, 1.15 g, 1.0 eq.) by hydrogenation (Pd/C, H2, RT, overnight) in ethyl acetate yielded Intermediate 14-11 (850 mg, 94%). 14-11 (300 mg, 0.9 mmol, 1.0 eq.) was converted to the corresponding chloride, 14-11′ using Oxalyl Chloride (3.0 mmol, 260 μL, 3.4 eq,) in 4 mL Toluene using DMF (0.2 μL, Catalytic quantity) at room temperature for 2 hours. Crude 14-11′ (0.17 mmol, 280 mg, 3.0 eq.) was used for N-acylation of Intermediate 13-0 (0.059 mmol, 200 mg, 1.0 eq.) using TEA (39.0 μL, 5.0 eq, 0.29 mmol), DMAP (10 mg, Catalytic quantity) in 3 mL of Toluene to obtain Lipid 14A. Purification of crude product by column chromatography (DCM: 10% MeOH in DCM) yielded 220 mg (76%) of purified Lipid 14A (98% by HPLC-CAD) and characterized by proton NMR and mass spectrometry (FIG. 4V-1 and FIG. 4V-2 for proton NMR and HPLC-CAD and Table 5 for Mass Spectrometry data).

### Synthesis of Lipid 15 by N-Acylation of Intermediate 13-11a Using the Corresponding Acid Chloride

### Synthesis of Lipid 15

Lipid 15 was synthesized as provided in scheme 18 below and as follows. Starting material, decan-4-ol, 13-29 (10.0 g, 63.0 mmol) was acylated with succinic acid, 13-30 (12.6 g, 126 mmol, 2.0 eq) using DMAP (7.7 g, 63 mmol, 1 eq) and pyridine (5.0 ml) in 5 mL THF and 15 mL DCM to obtain intermediate 13-31. Crude product was purified by column chromatography (3×) to obtain 8.9 g (55%) of pure acid intermediate 13-31. Intermediate 13-31 (1.26 g, 4.9 mmol) was converted to the acid chloride intermediate 13-31′ using oxalyl chloride (1.43 mL, 3.4 eq, 16.66 mmol) and DMF (50 μL, catalytic quantity), in 5 mL Benzene. Product showed only one spot on TLC (as methyl ester) and was used without further purification for acylation (step 3) of intermediate 13-11b. Intermediate 13-11b (275 mg, 0.39 mmol) was acylated with crude acid chloride, 13-31′ (324 mg, 3.0 eq, 1.17 mmol), TEA (270 μL, 5.0 eq, 1.95 mmol), DMAP (20 mg, catalytic quantity) in 10 mL of benzene to obtain Lipid 15. Crude product was purified by column chromatography (2×) to yield 230 mg g (64%) of pure Lipid 15 (99% purity by LC-ELSD) and characterized by proton NMR and Mass Spectrometry (see FIG. 4N-1 for Lipid 15 NMR spectrum, FIG. 4N-2 for Lipid 15 LC-MS, and Table 5 for product mass).

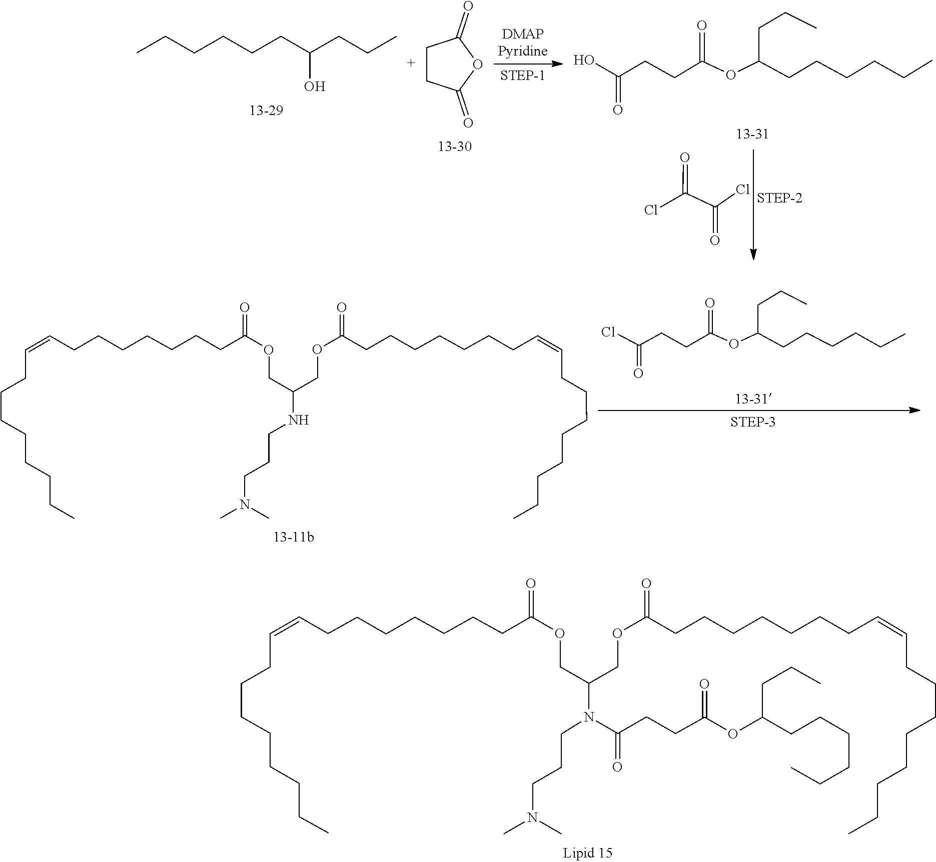

### Synthesis of Lipid 16

Lipid 16 was synthesized as provided in scheme 19 below and as follows. Starting material, octan-3-ol, 13-48 rac (3 g, 23 mmol) was acylated with succinic acid, 13-30 (46.08 mmol, 4.61 g, 2.0 eq) using DMAP (23.04 mmol, 2.8 g, 1.0 eq,) and pyridine (5.0 ml) in 5 mL THF and 15 mL DCM to obtain intermediate 13-31. Crude product was purified by column chromatography (1×) to obtain 3.4 g (64%) of pure acid intermediate 13-47 rac. Intermediate 13-47 rac (300 mg, 0.42 mmol) was converted to the acid chloride intermediate 13-47′ rac using oxalyl chloride (0.38 mL, 4.4 mmol, 3.4 eq.) and DMF (2 μL, catalytic quantity). Product showed only one spot on TLC (as methyl ester) and was used without further purification for acylation (step 3) of intermediate 13-11b. Intermediate 13-11b (270 mg, 0.38 mmol) was acylated with crude acid chloride, 13-47′ rac (0.42 mmol, 300 mg, 3.0 eq.), TEA (260 μL, 5.0 eq, 1.9 mmol), DMAP (20 mg, catalytic quantity) in 5 mL of toluene to obtain Lipid 16. Crude product was purified by column chromatography (1×) to yield 165 mg (47%) of pure Lipid 16 (99% purity by LC-ELSD) and characterized by proton NMR and Mass Spectrometry (see FIG. 4O-1 for Lipid 16 NMR spectrum, FIG. 4O-2 for Lipid 16 LC-MS, and Table 5 for product mass).

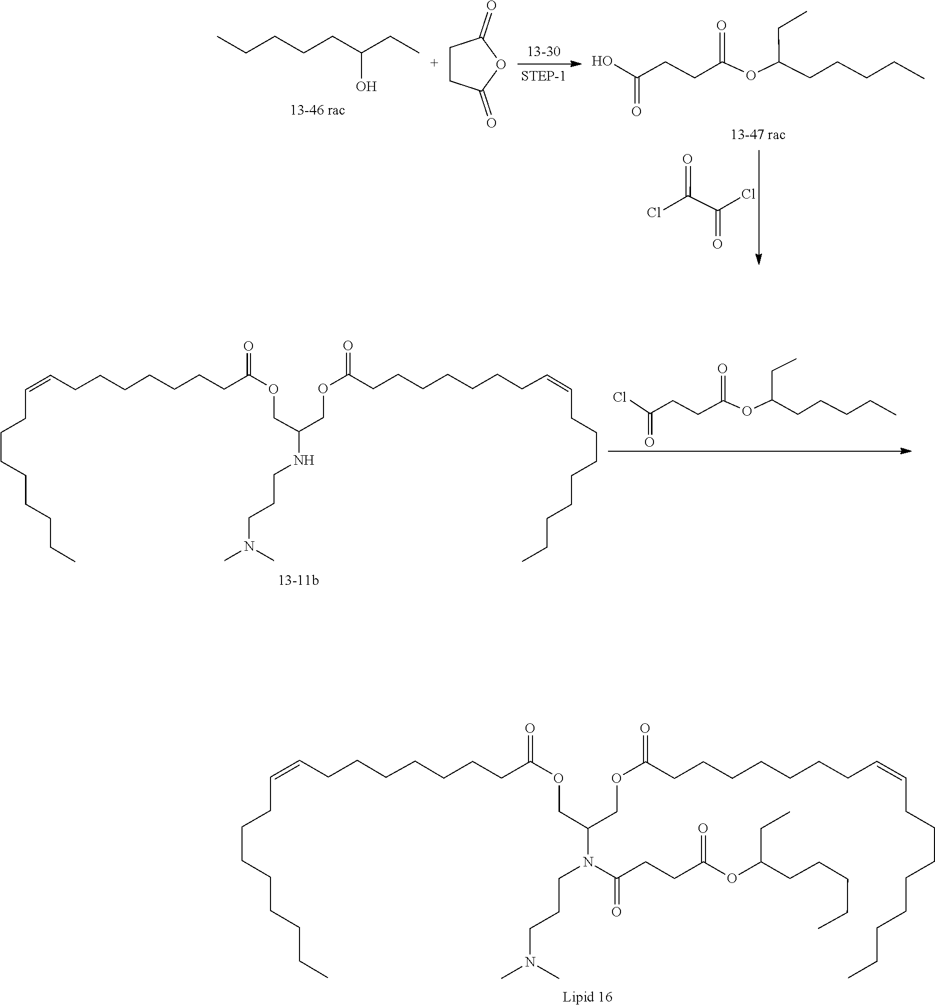

### Synthesis of Lipid 17

Lipid 17 was synthesized as provided in scheme 20 below. Octanedioic acid, 13-51 (5.0 g, 2.0 eq, 28.5 mmol) was mono-acylated with decane-3-ol, 13-29 (2.75 mL, 1.0 eq, 14.3 mmol) using EDCI (3.29 g, 1.2 eq, 17.2 mmol), DMAP (160 mg, 0.12 eq, 1.72 mmol) and TEA (9.96 mL, 5.0 eq, 71.5 mmol) in 50 mL of DCM/DMF (1:1 v/v) (50 mL) at room temperature overnight to obtain free acid 13-53. Crude product was purified by column chromatography (1×) to obtain 1.06 g (28%) of purified 13-53. Acid 13-53 (1.06 g, 2 eq., 3.7 mmol) was reacted with dihydroxyacetone (152 mg, 1.0 eq, 1.7 mmol) using EDCI (816 mg, 2.5 eq, 4.25 mmol), DMAP (50 mg, 0.25 eq, 0.43 mmol) and DIPEA (740 μL, 2.5 eq, 4.3 mmol) in 15 mL DCM at room temperature overnight to obtain ketone 13-54. Crude product was purified by column chromatography (1×) to obtain 890 mg (69%) of purified 13-54. Reductive amination of 13-54 (890 mg, 1.0 eq, 1.3 mmol) with amine, 15-3 (327 μl, 2.0 eq, 2.6 mmol) using acetic acid (150 μL, 2.0 eq, 2.6 mmol) and sodium borohydride triacetate, Na(OAc)₃BH (331 mg, 1.2 eq, 1.5 mmol) in 20 mL DCM (20 ml) at room temperature for 3 hours yielded intermediate 13-55. Crude product was purified by column chromatography (1×) to obtain purified 13-55 (470 mg, 47%). N-acylation of intermediate 13-55 using acid 13-31 and reaction conditions reported for N-acylation of Lipid 15 synthesis resulted in Lipid 17.

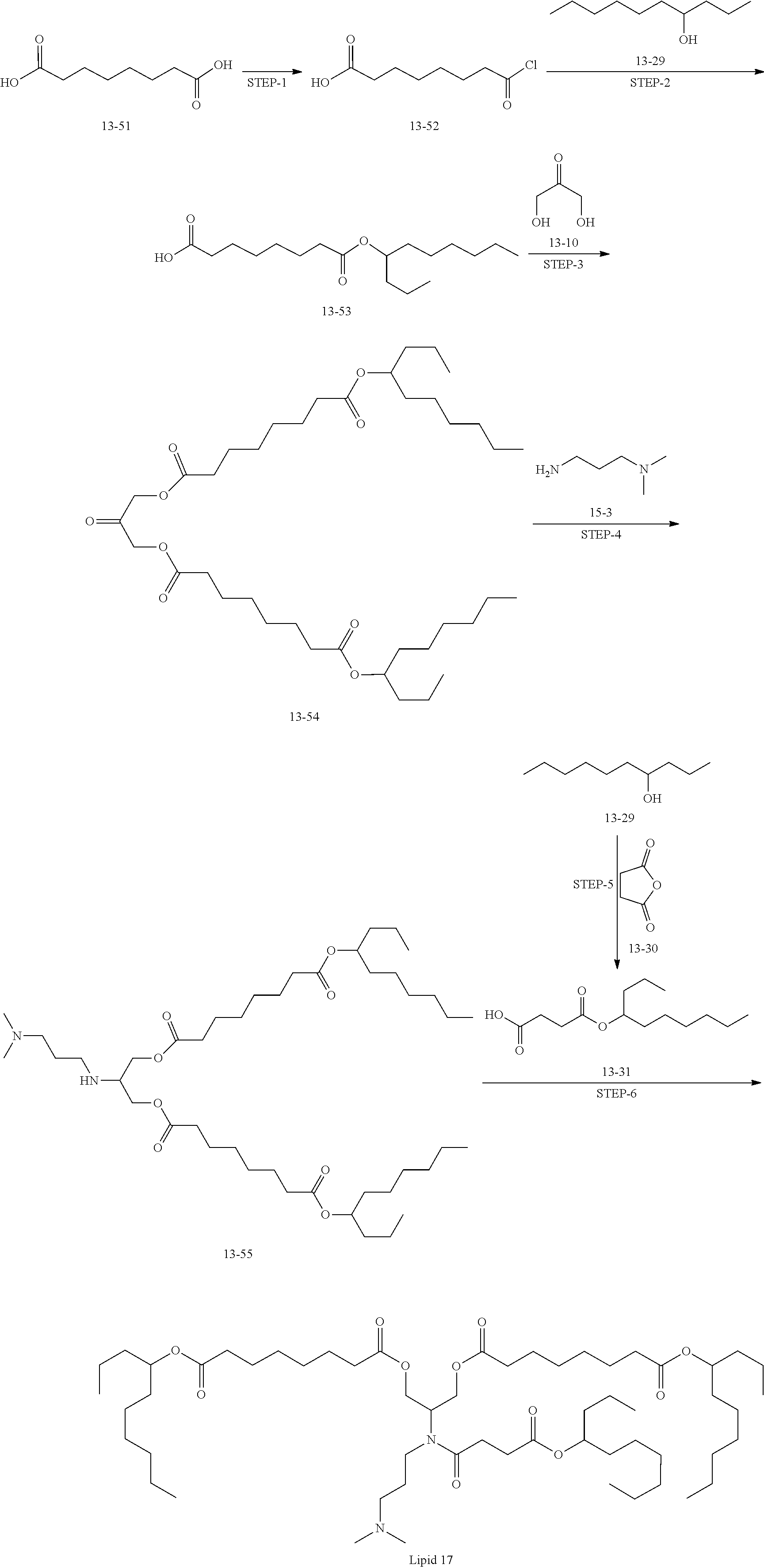

### Synthesis of Lipid 17A

Starting material 4-hydroxydecanol (63.1 mmol, 10 g, 1 eq.) was acylated with succinic anhydride, 13-30 (12.64 g, 2.0 eq, 12.2 mmol) using EDCI (2.65 g, 2.5 eq, 126.3 mmol), DMAP (7.7 g, 1.0 eq, 63.1 mmol) and Pyridine (17 mL) in a mixture of 17 ml of THF and 50 mL of DCM at room temperature, overnight to obtain 8.8 g (54%) of acid intermediate, 13-31. Starting material, 1,3-dihydroxyacetone, 13-10 (8.32 mmol, 0.75 g, 1 eq.) was diacylated with intermediate 13-31 (20.8 mmol, 5.36 g, 2.5 eq,) using EDCI (3.98 g, 2.5 eq, 20.8 mmol) and DMAP (0.203 g, 0.2 eq, 1.6 mmol) in 20 mL of DCM at RT, overnight to obtain 1.89 g (40%) of ketone intermediate 13-70. Intermediate 13-70 (3.2 mmol, 31.87 g, 1 eq.) was converted to diamine intermediate 13-71 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3 hours. Purification of crude product by 2× column chromatography (10% MeOH in DCM) yielded 500 mg (23%) of purified Intermediate 13-71. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-71 (0.3 mmol, 0.2 g, 1 eq.) using TEA (1.5 mmol, 3.98 g, 5.0 eq,) and DMAP (20 mg, catalytic quantity) in 4 mL of Toluene at room temperature overnight to yield Lipid 17A. Crude product was purified 2× by column chromatography (DCM: 10% MeOH in DCM) to obtain 165 mg (60%) of pure Lipid 17A (>99% by HPLC-CAD) and characterized by proton NMR and mass spectrometry (FIG. 4W-1 and FIG. 4W-2 for proton NMR and HPLC-CAD and Table 5 for Mass Spectrometry data).

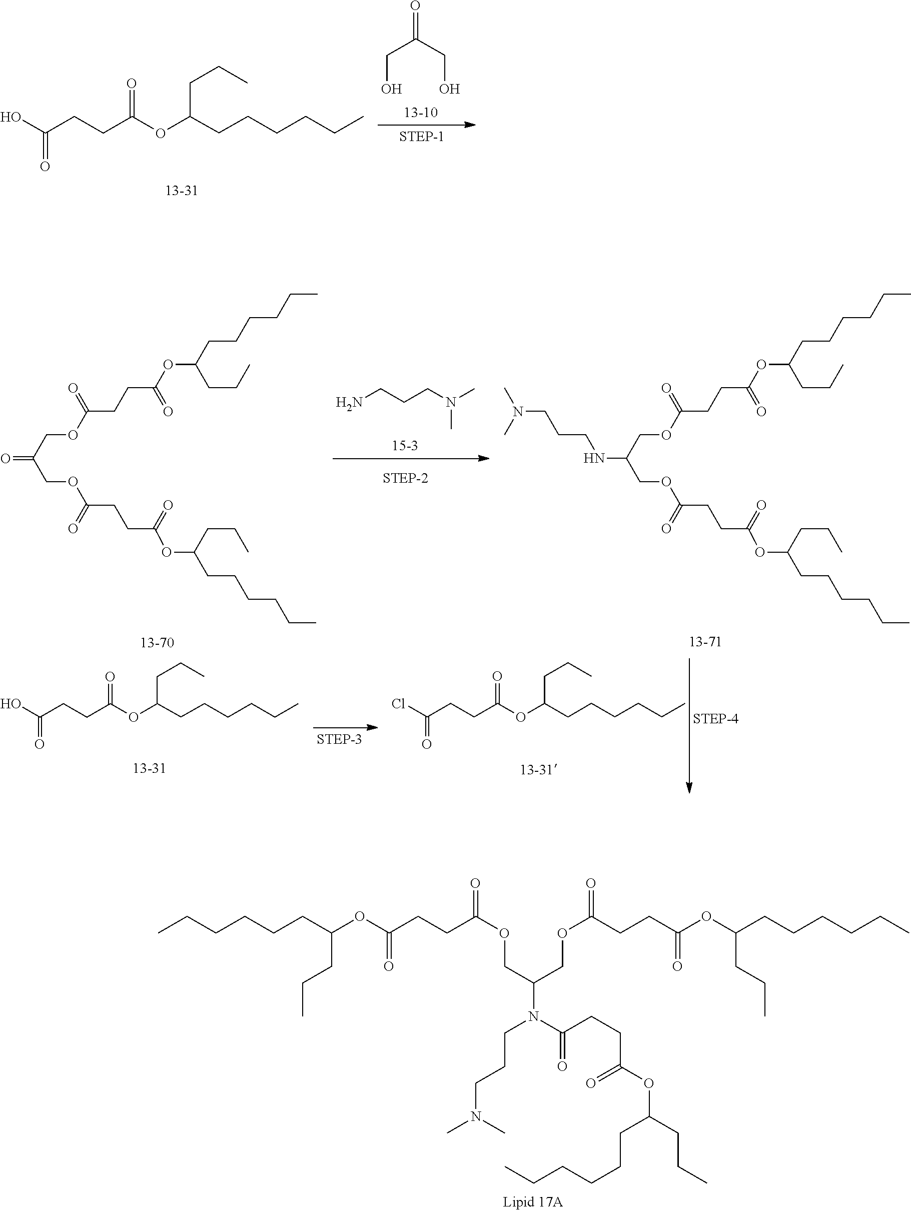

### Synthesis of Lipid 18 and its Isomer

An isomer of lipid 18 was synthesized as provided in scheme 21-1 below. Lipid 18 was synthesis using methods analogous to those reported for Lipid 17 by replacing decan-3-ol with octane-2-ol in the Step 1.

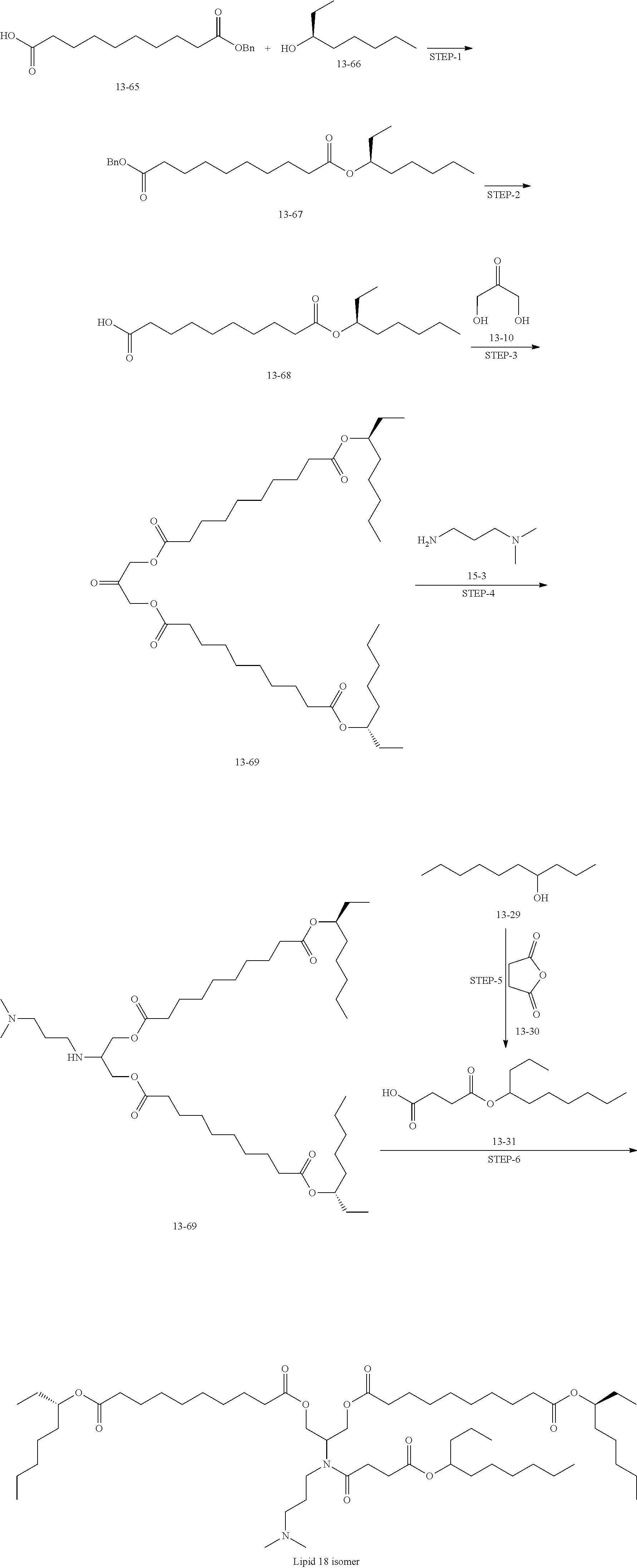

Lipid 18 as a racemic mixture was synthesized as provided in scheme 21-2 below.

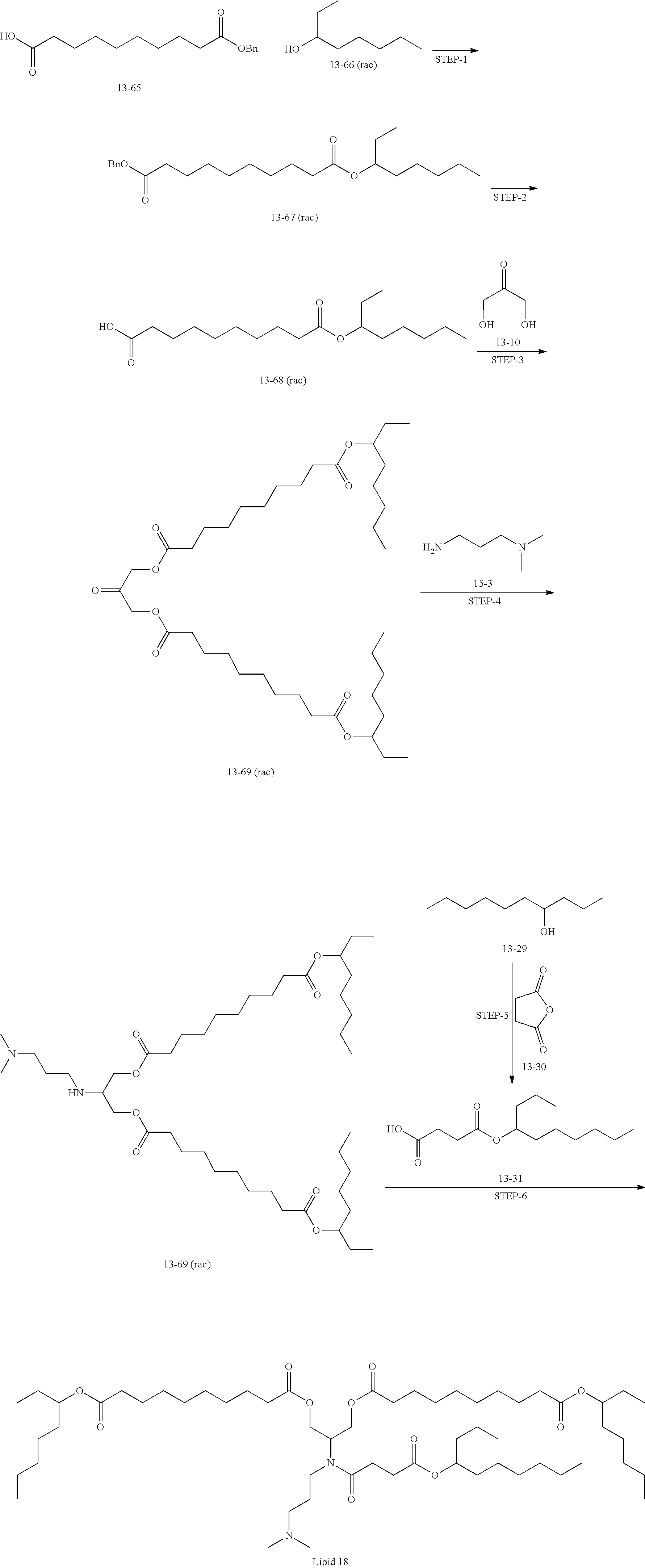

### Synthesis of Lipid 18A

Starting material 4-hydroxydecanol, 13-29 (31.6 mmol, 5 g, 1 eq.) was acylated with adipic acid, 13-72 (10.4 g, 2.0 eq, 63.2 mmol) using EDCI (37.90 mmol, 7.3 g, 1.2 eq,), DMAP (0.5 g, 0.12 eq, 3.8 mmol, 0.5 g, 0.12 eq,) and TEA (158 mmol, 22 mL, 5.0 eq.) in a mixture of 50 mL of DCM (50 mL) 50 mL DMF (50 mL) at room temperature, RT, overnight to obtain 9.8 g (90%) of acid intermediate, 13-74. Starting material, 1,3-dihydroxyacetone, 13-10 (11.09 mmol, 1.0 g, 1.0 eq.) was diacylated with acid Intermediate 13-74 (27.7 mmol, 7.93 g, 2.5 eq.) using EDCI (27.7 mmol, 5.32 g, 2.5 eq,) and DMAP (3.58 g, 0.2 eq, 2.2 mmol) in 30 mL at room temperature overnight to obtain 1.18 g (17%) of ketone intermediate 13-75. Intermediate 13-75 (3.2 mmol, 1.16 g, 1.0 eq.) was converted to diamine intermediate 13-76 by reductive amination with N,N-dimethylamino-3-aminopropane, 15-3 (6.5 mmol, 0.66 g, 2.0 eq.) using Na(OAc)₃BH (1.38 g, 2.0 eq, 6.5 mmol), acetic acid (0.37 mL, 2.0 eq, 6.5 mmol) in 15 mL of DCM (15 mL) at room temperature for 3-4 hours. Purification of crude product by 2× column chromatography (10% MeOH in DCM) yielded 660 mg (50%) of purified Intermediate 13-76. Additional quantity of acid Intermediate 13-31 (19.4 mmol, 0.24 g, 1 eq.) was converted to the corresponding acid chloride 13-31′ using Oxalyl chloride (0.92 mmol, 0.26 mL, 3.4 eq.) and DMF (20 μL, catalytic quantity) in 3 mL of Toluene at room temperature for 2 hours and crude acid chloride 13-31 (0.9 mmol, 0.23 g, 3 eq.) was used for N-acylation of diamine intermediate 13-76 (0.3 mmol, 0.2 g, 1 eq.) using TEA (1.5 mmol, 3.98 g, 5.0 eq,) and DMAP (20 mg, catalytic quantity) in 4 mL of Toluene at room temperature overnight to yield Lipid 18A. Crude product was purified 2× by column chromatography (DCM: 10% MeOH in DCM) to obtain 175 mg (60%) of pure Lipid 18A (>99% by HPLC-CAD) and characterized by proton NMR and mass spectrometry (FIG. 4X-1 and FIG. 4X-2 for proton NMR and HPLC-CAD and Table 5 for Mass Spectrometry data).

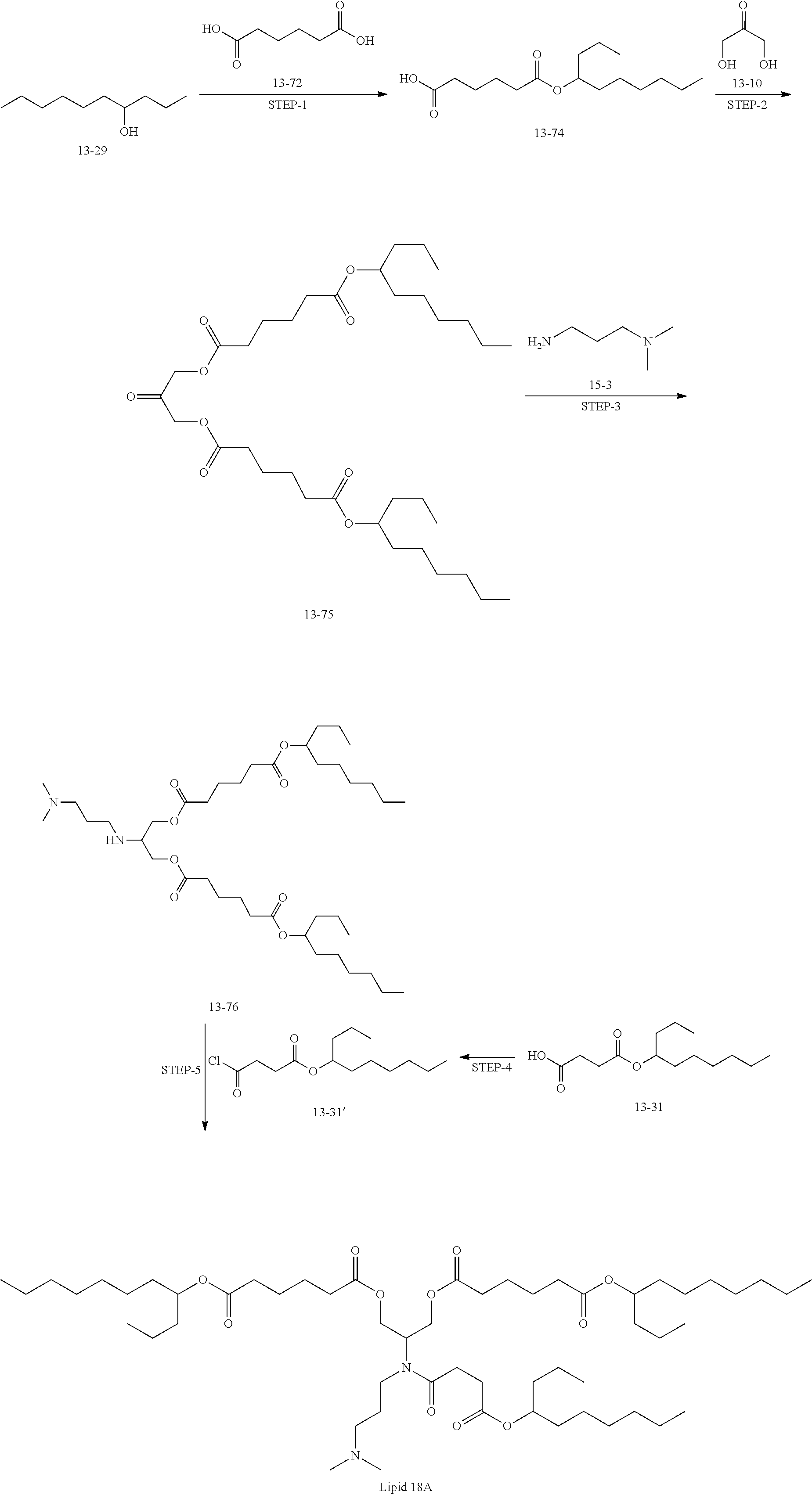

### Synthesis of Lipid 19

Lipid 19 was synthesized as provided in scheme 22 below and as follows. Starting material dihydroxyacetone (422 mg, 4.7 mmol) was acylated with compound 13-56 (3.0 g, 2.5 eq, 11.71 mmol) using EDCI (2.24 g, 2.5 eq, 11.71 mmol), DIPEA (2.0 mL, 2.5 eq, 11.71 mmol), and DMAP (115 mg, 0.2 eq, 0.94 mmol) in 10 mL DCM yielding 2.1 g (79%) of intermediate 13-57. Reductive amination of 13-57 (2.1 g, 1.0 eq, 3.7 mmol) with amine 15-3 (925 μL, 2.0 eq, 7.4 mmol) using acetic acid (430 μL, 2.0 eq, 7.4 mmol), Na(OAc)₃BH (923 mg, 1.2 eq, 4.44 mmol) in 10.0 mL DCM yielding 1.55 g (65%) of intermediate 13-58. Intermediate 13-31 was produced as described in the synthesis of Lipid 9 and Lipid 15 earlier. N-acylation of intermediate 13-58 (484 mg, 1.0 eq, 0.74 mmol) with 13-31 (380 mg, 2.0 eq, 1.48 mmol) using EDCI (291 mg, 2.0 eq, 1.48 mmol), DIPEA (247 μL, 2.0 eq, 1.48 mmol), and DMAP (45 mg, 0.5 eq, 0.37 mmol) in 4.0 mL DCM at room temperature, overnight yielded 423 mg (63%) of pure lipid 19 (>99% purity).

See FIG. 4P-1 for Lipid 19 NMR spectrum, FIG. 4P-2 for Lipid 19 reverse phase LC-ELSD chromatogram, and Table 5 for product mass.

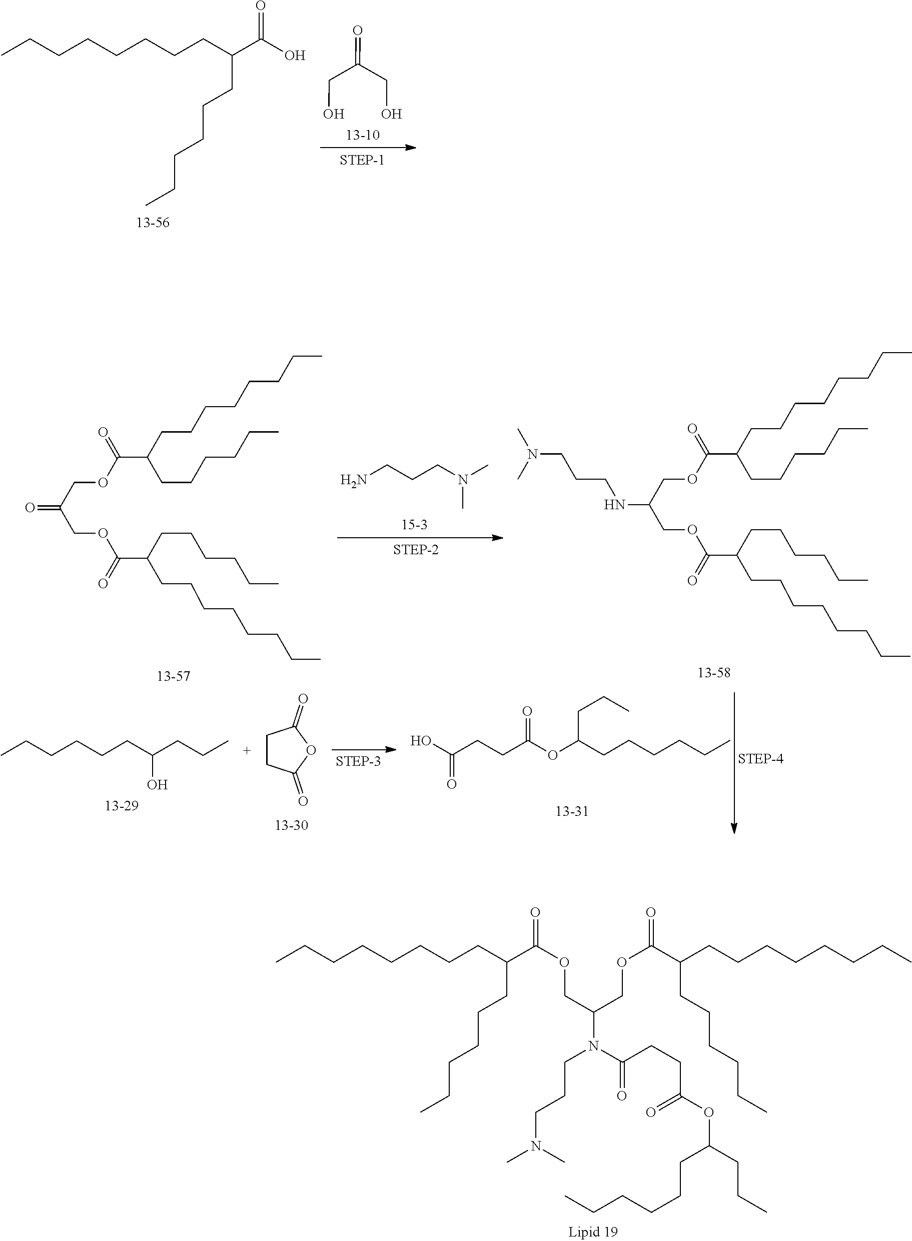

### Synthesis of Lipid 19A

Starting material tertiary-butyl protected suberic acid 13-51 (8.6 mmol, 2.0 g, 1 eq.) was esterified using 4-hydroxydecanol 13-52 (13.02 mmol, 2.06 g, 1.5 eq) using EDCI (13.02 mmol, 2.49 g, 1.5 eq), DMAP (4.3 mmol, 0.53 g, 0.5 eq) and DIPEA (13.02 mmol, 1.68 g, 1.5 eq) in 20 mL of dichloromethane at room temperature for 4 hours. yielding 2.83 g (88%) of protected intermediate 13-53. Intermediate 13-53 (4.05 mmol, 1.5 g, 1.0 eq.) was deprotected using 4N HCl in 10 mL dioxane at room temperature overnight yielding 1.07 g (84%) of acid intermediate 13-54.

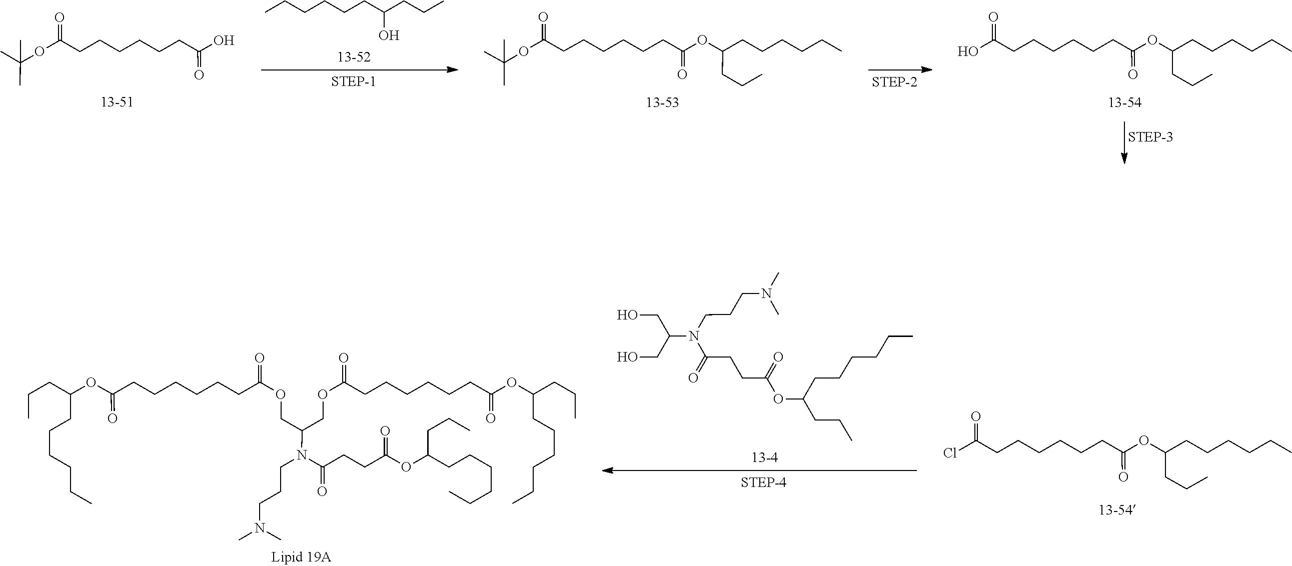

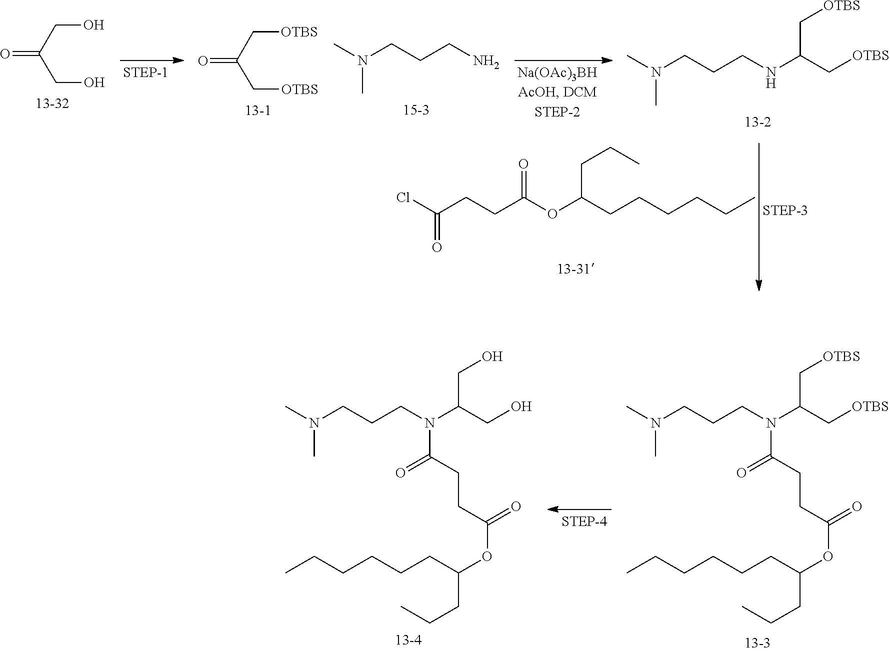

Protection of starting material dihydroxyacetone 13-32 (111 mmol, 10 g, 1 eq.) using tert-butyltrimethylsilyl chloride TBSCl (332 mmol, 50 g, 3.0 eq), TEA (148 mmol, 160 mL, 10.34 eq,) and DMAP (23 mmol, 2.80 g, 0.21 eq.) in 420 mL DCM (420.0 mL) at room temperature overnight yielded protected intermediate 13-1. A second batch of 13-1 was produced from 50 g (0.56 mol, 1.0 eq) of additional 13-32 using TBSCl (1.68 mol, 250 g, 3.0 eq), TEA (5.6 mol, 400 mL, 10.34 eq,), DMAP (0.11 mol, 13.7 g, 0.21 eq,) in 800 mL of DCM at room temperature overnight. Crude products from the two batches were reductively aminated separately and combined prior to purification. First batch of 13-1 (176 g, 552.0 mmol) was reductively aminated using N,N-dimethylaminopropyl amine 15-3, (1104.0 mmol, 139 mL, 2.0 eq.), Acetic acid (1104.0 mmol, 64 mL, 2.0 eq.), and Na(OAc)₃BH (662.0 mmol, 135 g, 1.2 eq.), in 1.5 L of dichloromethane at room temperature for 3 hours. The second batch of 13-1 (36.8 g, 115.4 mmol) was reductively aminated using N,N-dimethylaminopropyl amine 15-3, (230.8 mmol, 29 mL, 2.0 eq.), Acetic acid (230.8 mmol, 13.4 mL, 2.0 eq,), and Na(OAc)₃BH (138.5 mmol 28.2 g, 1.2 eq.), in 300 mL of dichloromethane at room temperature for 3 hours. Combined crude product from the two batches was purified by filter column chromatography on silica column eluting with DCM and (10% MeOH in DCM+1% NH₄OH) to obtain desired product yielding 17 g pure intermediate 13-2 based on TLC.

Two batches of 13-2 were separately deprotected using identical reactions conditions; each consisting of 13-2 (300 mg, 0.465 mmol) in HF-pyridine, (4.65 mmol, 0.42 mL, 10.0 eq.) and 2 mL THF at room temperature for 2 hours.

Two batches of acid intermediate 13-54 were separately converted to the corresponding acid chloride using identical reaction conditions; each consisting of 13-54 (881 mg, 2.8 mmol) using oxalyl chloride (9.5 mmol, 0.8 mL, 3.4 eq,), DMF (100 μL, catalytic quantity) in 6.0 mL of toluene at room temperature for 2 hours.

Crude di-hydroxy intermediate 13-4 (194 mg, 0.465 mmol) and crude acid chloride 13-54′ (2.8 mmol, 881 mg, 6.0 eq.), were combined with TEA (4.65 mmol, 0.65 mL, 10.0 eq,) in 8.0 mL of toluene at room temperature overnight. Crude product was purified on ISCO column chromatography on silica column eluting with DCM and 10% MeOH in DCM. Column purification was repeated after product isolation yielding 247 mg (53%) of 76% pure (HPLC-CAD) Lipid 19A. Product was re-purified on ISCO column chromatography on silica column eluting with DCM and 10% MeOH in DCM. yielding 122 mg of >99% purity (HPLC-CAD) Lipid 19A (see FIG. 4AI-1 and FIG. 4AI-2 for characterization by proton NMR, and LC-CAD purity and Table 5 for Mass Spectrometry data).

### Synthesis of Lipid 20

Lipid 20 was synthesized as provided in scheme 23 below and as follows. Monoprotected succinic acid, 13-59 (2.0 g, 1.0 eq, 9.65 mmol) was reduced to the corresponding alcohol using Borane-dimethyl sulfide (6.2 mL, 7.0 eq, 67.0 mmol) at 0-5° C., 1 hr followed by room temperature reaction overnight. Crude product was purified by column chromatography (2×) yielding 1.3 g (71%) of pure compound 13-60. Intermediate 13-60 (1.3 g, 1.3 eq, 6.7 mmol) was used to acylate acid 13-56 (1.51 mL, 1.0 eq, 5.0 mmol) using EDCI (1.63 g, 1.7 eq, 8.5 mmol), DIPEA (1.48 mL, 1.7 eq, 8.5 mmol) and DMAP (98 mg, 0.17 eq, 0.85 mmol) in 10.0 mL DCM at room temperature overnight. Crude product was purified by column chromatography (1×) yielding 1.88 g (65%) of pure intermediate 13-61. Subsequent deprotection by hydrogenation on Pd/C/Hydrogen gas (400 mg) in methanol yielded 1.42 g of free acid 13-62 (99%) of crude product. Crude 13-62 (1.32 g, 2.2 eq, 4.2 mmol) was used to acylate dihydroxyacetone, 13-10 (172 mg, 1.0 eq, 1.9 mmol), EDCI (958 mg, 2.6 eq, 5.0 mmol), DIPEA (870 μL, 2.6 eq, 5.0 mmol), and DMAP (56 mg, 0.26 eq, 0.5 mmol) in 10.0 mL DCM at room temperature, overnight to obtain ketone 13-63. Crude product was purified by column chromatography to obtain 120 mg (3.8%) of pure 13-63. Reductive amination of 13-63 (120 mg, 1.0 eq, 0.16 mmol) with amine 15.-3 (42 μl, 2.0 eq, 0.32 mmol) using acetic acid (18 μL, 2.0 eq, 7.8 mmol) and Na(OAc)₃BH (41 mg, 1.2 eq, 0.19 mmol) in 3 mL DCM at room temperature of 3 hours yielded intermediate 13-64. Crude product was purified by column chromatography (1×) to obtain 23 mg (17%) of purified intermediate 13-64. N-acylation of 13-64 (23 mg, 1.0 eq, 0.028 mmol) with acid 13-31 (8.7 mg, 1.2 eq, 0.034 mmol) using EDCI (6.4 mg, 1.2 eq, 0.034 mmol), DIPEA (5.8 μL, 1.2 eq, 0.034 mmol) and DMAP (1 mg, cat) in 1.5 mL DCM at room temperature overnight yielded Lipid 20. Crude product was purified by column chromatography (1×) to obtain 21 mg (70%) of pure lipid 20 (99%).

See FIG. 4Q-1 for Lipid 20 NMR spectrum, FIG. 4Q-2 for Lipid 20 reverse phase LC-ELSD chromatogram, and Table 5 for product mass.

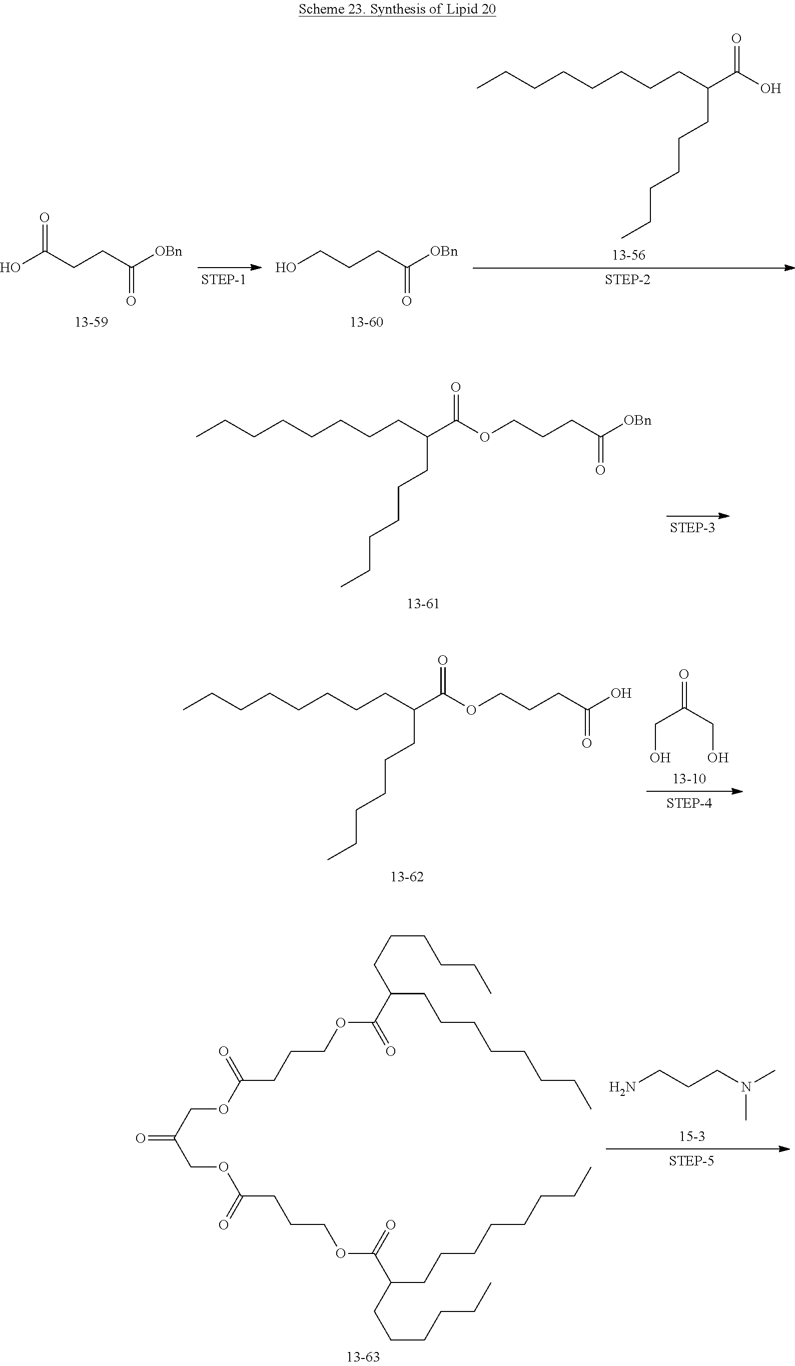

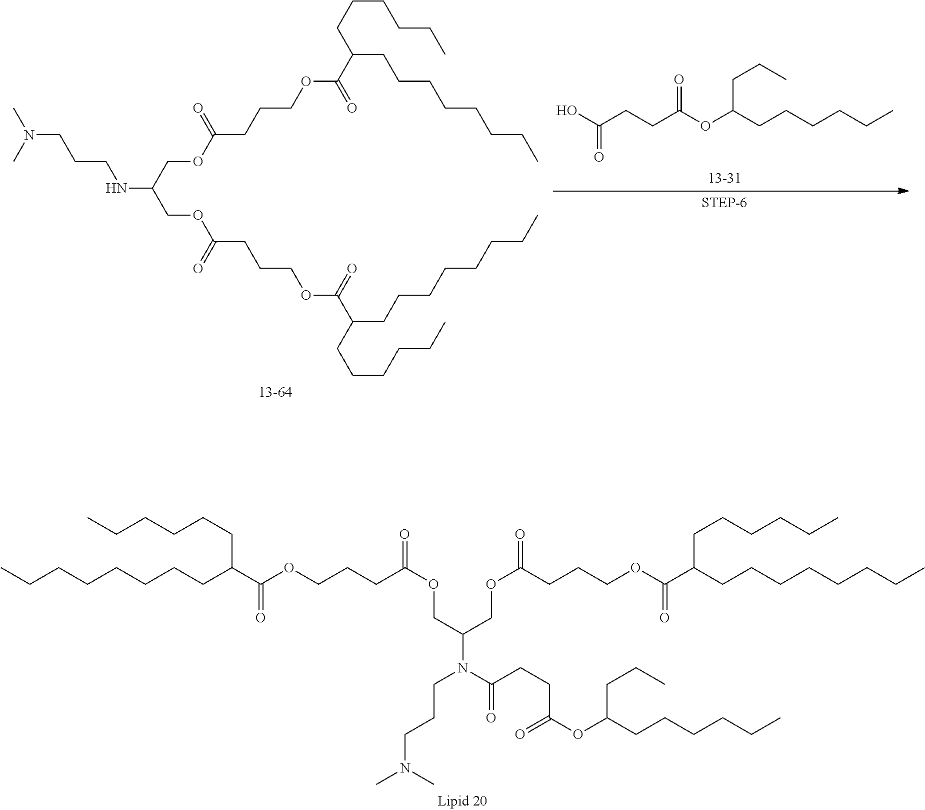 [SMILES: CCCCCCCCC(CCCCCC)C(=O)OCCCC(=O)OCC(COC(=O)CCCOC(=O)C(CCCCCC)CCCCCCCC)NCCCN(C)C ; CCCCCCC(CCC)OC(=O)CCC(=O)O ; CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

### Synthesis of Lipid 20A

Starting material tertiary-butyl protected sebacic acid 14-19 (15.5 mmol, 4.0 g, 1 eq.) was esterified using 4-hydroxydecanol 14-20 (23.25 mmol, 3.7 g, 1.5 eq) and EDCI (23.25 mmol, 4.5 g, 1.5 eq), DMAP (7.75 mmol, 0.95 g, 0.5 eq) and DIPEA (23.25 mmol, 4 mL, 1.5 eq.) in 20 mL of dichloromethane at room temperature overnight yielding 4.1 g (66%) of protected intermediate ester 14-21. Intermediate 14-21 (10.3 mmol, 4.1 g, 1.0 eq.) was deprotected using 4N HCl in 15 mL dioxane at room temperature overnight yielding 2.7 g (77%) of acid intermediate 14-22.

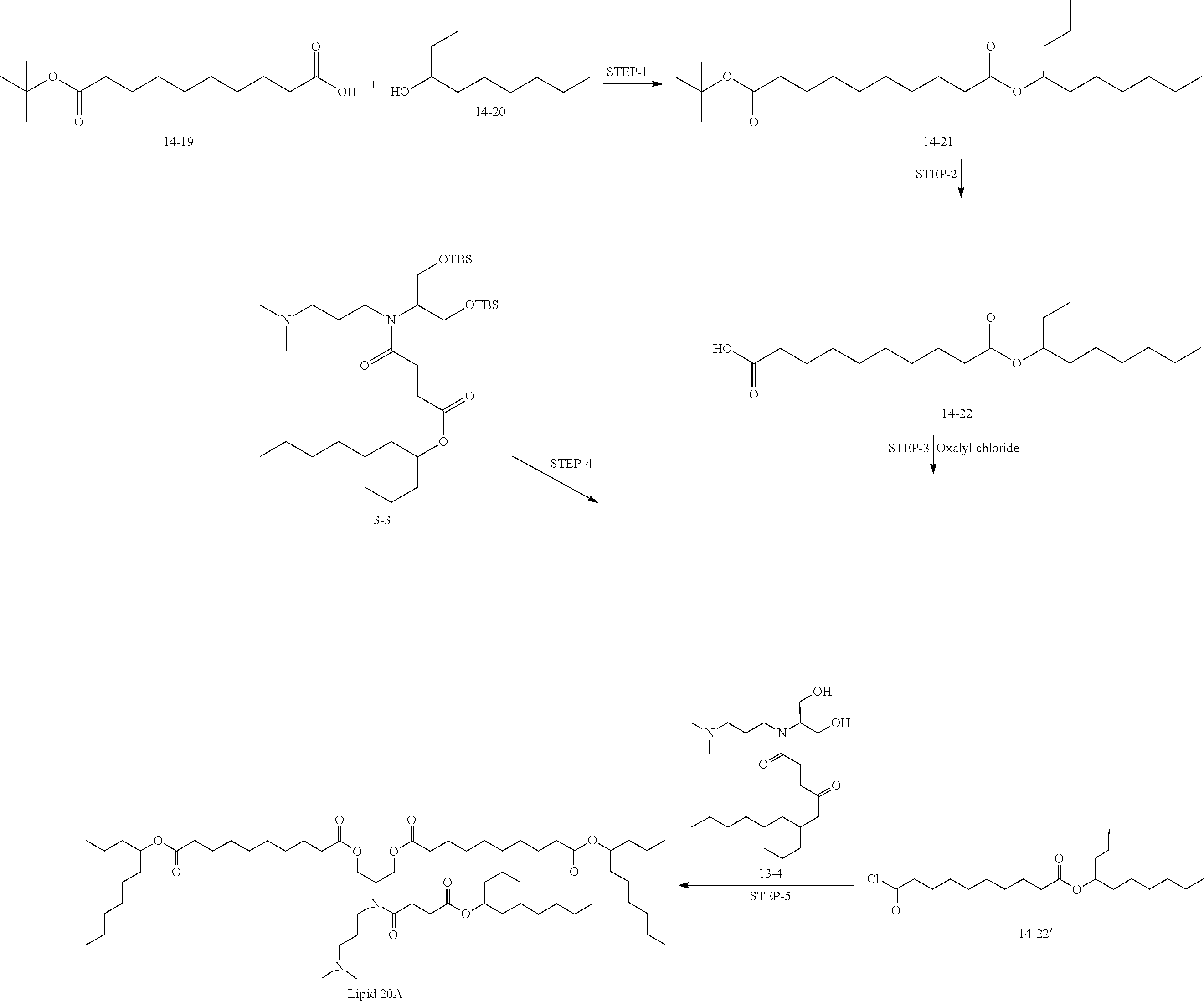

Protected intermediate 13-3 (400 mg, 0.62 mmol, 1 eq.) was treated with Hydrogen Fluoride/Pyridine (15.5 mmol, 1.11 mL, 25.0 eq.) in 6.0 mL THF at room temperature for 2 hours and deprotection was confirmed by TLC and mass spectrometry. Acid intermediate 14-22 (3.72 mmol, 1.27 g, 1 eq.) was converted to the corresponding acid chloride 14-22′ using oxalyl chloride (12.6 mmol, 1.1 mL, 3.4 eq.) and DMF (40 μL, catalytic quantity) in 5.0 mL of Toluene at room temperature for 2 hours and conversion to chloride intermediate by TLC.

Crude di-hydroxy intermediate 13-4 (258 mg, 0.62 mmol, 1 eq.) and crude acid chloride 14-22′ (3.72 mmol, 1.34 g, 6.0 eq.) were combined with TEA (6.2 mmol, 0.87 mL, 10.0 eq) in 5 mL of toluene at room for 2 hours. Crude product was purified on ISCO column chromatography on silica column eluting with DCM and 10% MeOH in DCM yielding 300 mg of 96% pure (HPLC-CAD) Lipid 20A (see FIG. 4AJ-1 and FIG. 4AJ-2 for characterization by proton NMR, mass spectrometry and LC-CAD purity).

### Synthesis of Lipid 21 and its Isomer

An isomer of lipid 21 was synthesized as provided in scheme 24-1 below. Briefly, alcohol 13-78 was accessed by nucleophilic addition to aldehyde 13-77 using diethyl zinc (Step 1) and subsequently used in the ring opening addition to cyclic anhydride 13-52 to access intermediate 13-79. O-acylation of dihydroxyacetone with intermediate 13-79 using conditions described in Lipid 17 synthesis yielded ketone 13−80. Reductive amination of 13−80 with amine 15-3 using conditions described in the Lipid 17 synthesis yielded intermediate 13-81. Subsequent N-acylation of intermediate 13-81 with acid 13-31 using conditions analogous to those used for Lipid 9 synthesis afforded Lipid 21.

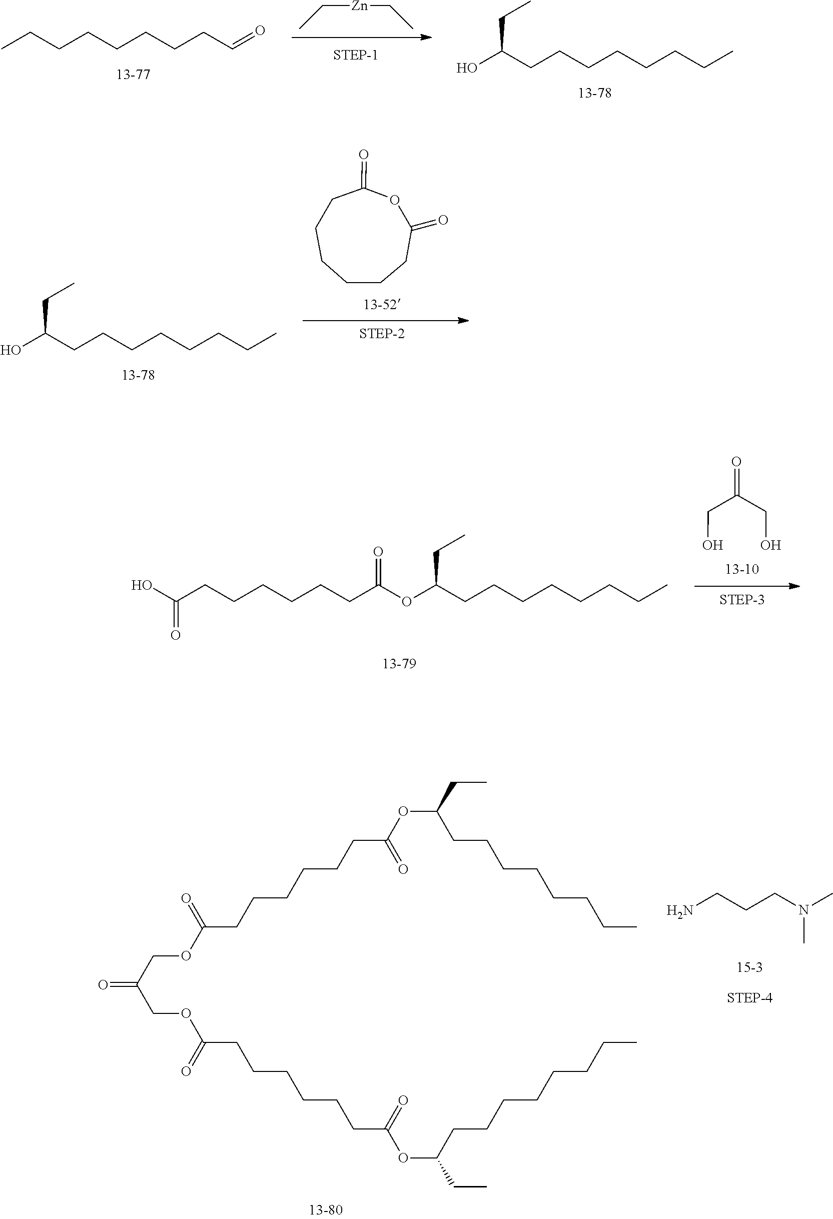

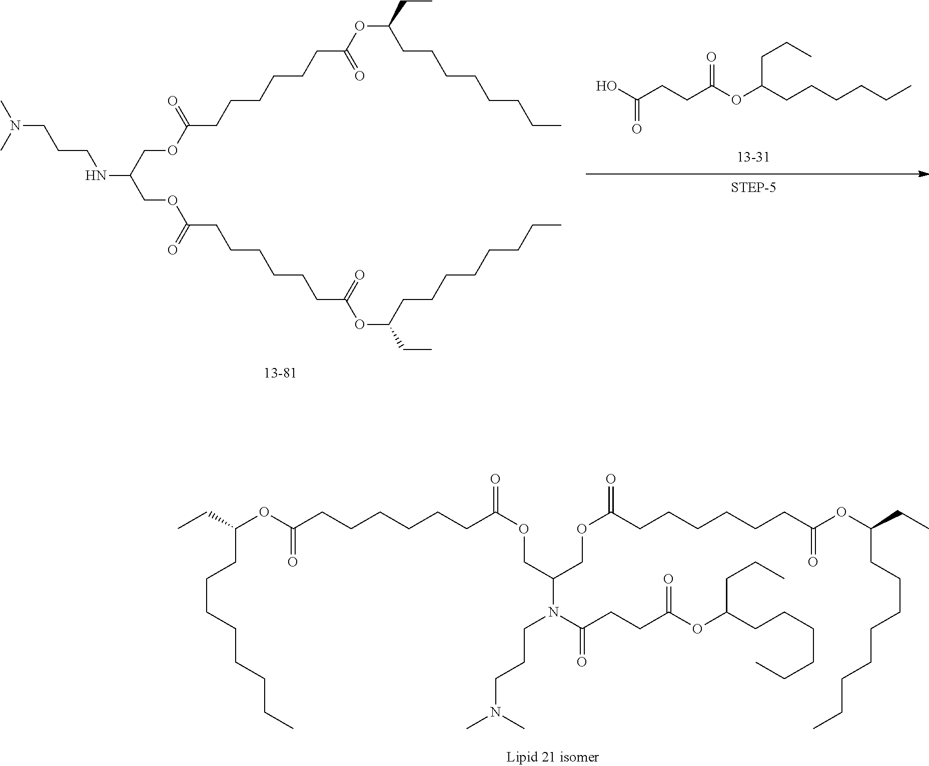

Lipid 21 as a racemic mixture was synthesized as provided in scheme 24-2 below. Briefly, Lipid 21 (racemate) was accessed using methods analogous to those described for Lipid 21 isomer except using ethyl lithium for accessing the racemic alcohol in Step 1.

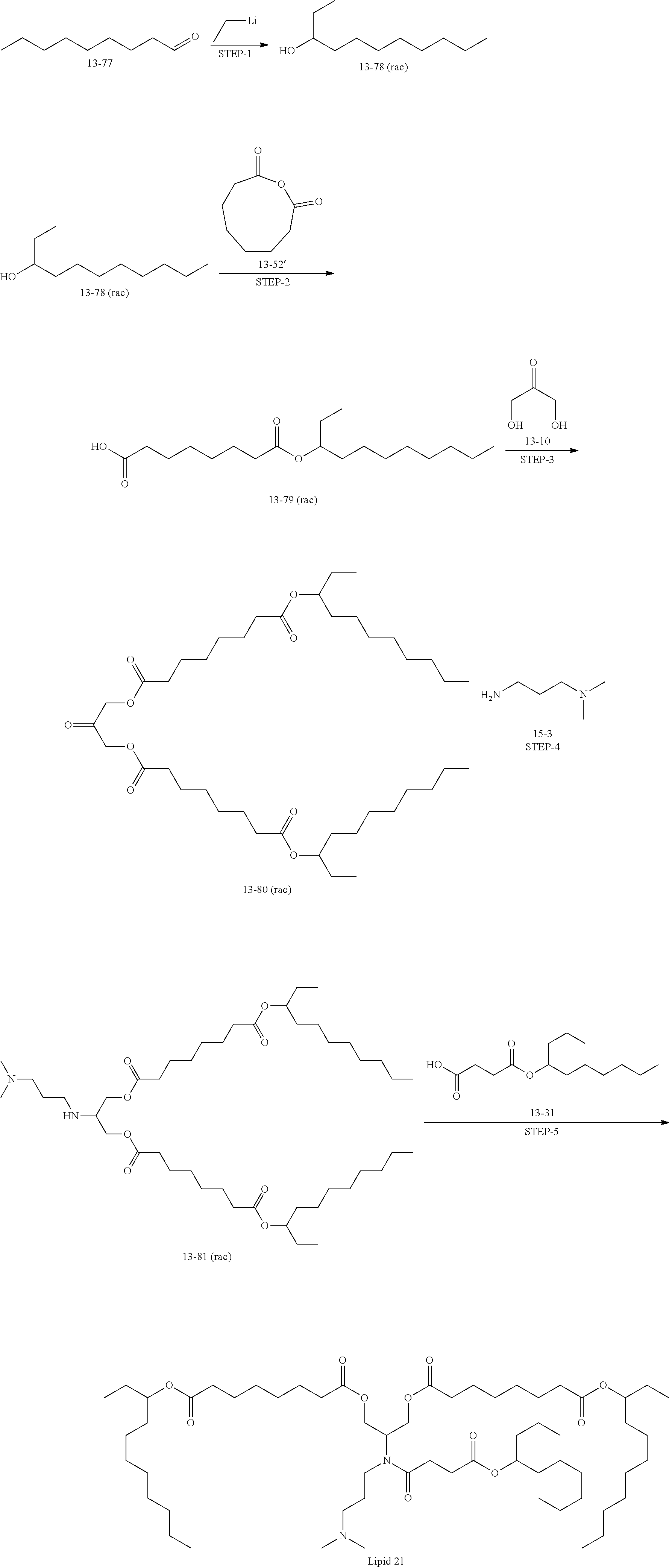

### Synthesis of Lipid 21A

Starting material 3-hydroxyoctanol, 13-66 (12.4 mmol, 1.61 g, 1 eq.) was acylated with sebacic acid 13-65 (24.8 mmol, 5.0 g, 2.0 eq.) using EDCI (14.9 mmol, 2.83 g, 1.2 eq.), DMAP (1.5 mmol, 183 mg, 0.12 eq.) and TEA (62.0 mmol, 8.6 mL, 5.0 eq.) in a mixture of 25 mL of DCM and 25 mL DMF at room temperature, RT, overnight to obtain 2.2 g (56%) of acid intermediate, 13-67. Starting material, 1,3-dihydroxyacetone, 13-10 (3.2 mmol, 286 mg, 1.0 eq.) was diacylated with acid Intermediate 13-67 (7.0 mmol, 2.2 g, 2.2 eq.) using EDCI (7.0 mmol, 1.33 g, 2.2 eq.), DIPEA (7.0 mmol, 1.22 mL, 2.2 eq.) and DMAP (1.6 mmol, 197 mg, 0.5 eq.) in 10 mL of DCM at room temperature overnight to obtain 1.4 g (65%) of ketone intermediate 13-68.

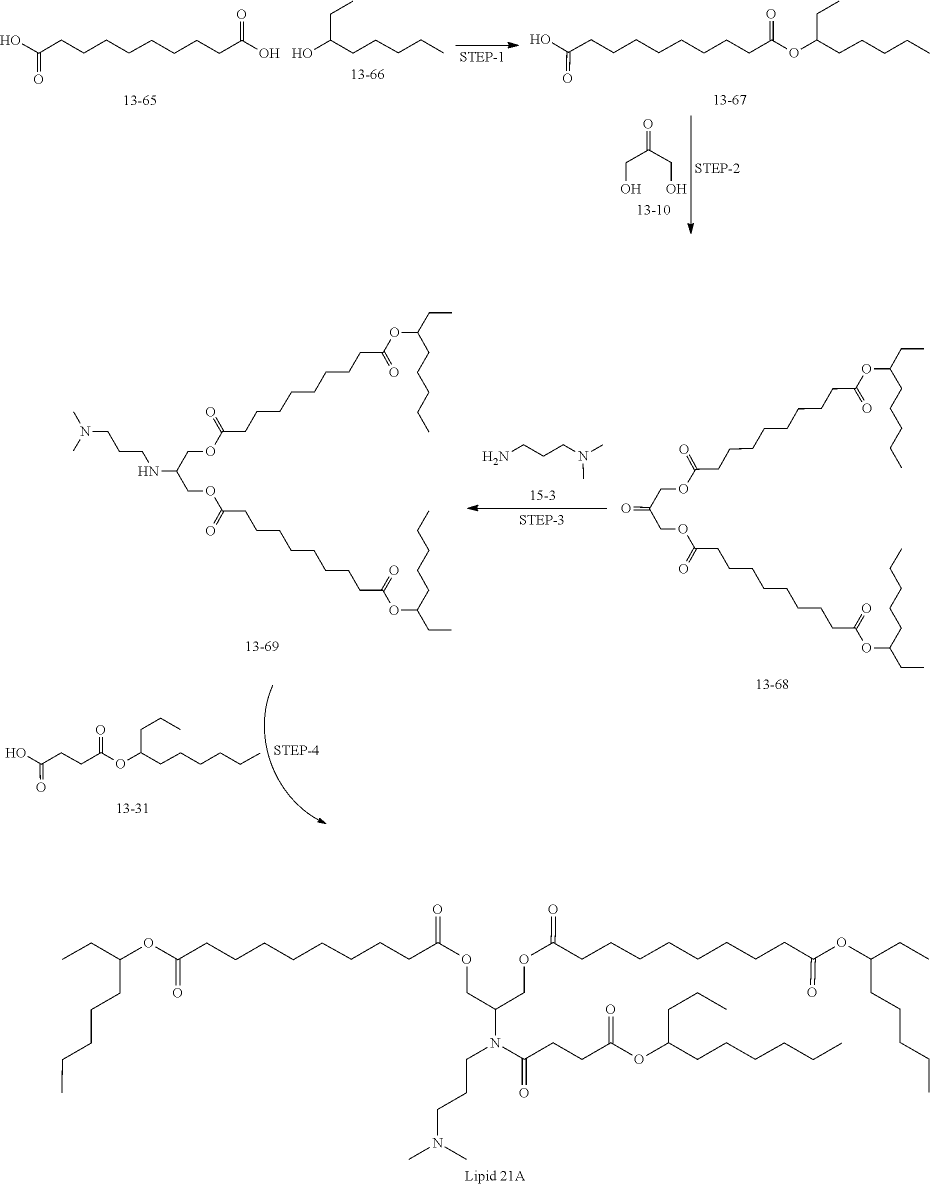

Intermediate 13-68 (2.05 mmol, 1.4 g, 1.0 eq.) was converted to diamine intermediate 13-69 by reductive amination with N,N-dimethylamino-3-aminopropane, 15-3 (514 μL, 2.0 eq, 4.10 mmol) using Na(OAc)₃BH (10 mg, 1.2 eq, 2.46 mmol)), acetic acid (236 μL, 2.0 eq, 4.10 mmol) in 5 mL of DCM at room temperature for 3 hours. Purification of crude product by silica column chromatography (10% MeOH in DCM, 1% NH₄OH) yielded 480 mg (32%) of purified Intermediate 13-69. Acid Intermediate 13-31 (1.86 mmol, 484 mg, 1 eq.) was converted to the corresponding acid chloride 13-31′ using Oxalyl chloride (540 μL, 3.4 eq, 6.38 mmol) and DMF (20 μL, catalytic quantity) in 3 mL of Toluene at room temperature for 2 hours and crude acid chloride 13-31′, (518 mg, 3.0 eq, 1.88 mmol), TEA, Toluene (3.0 mL), RT, Overnight was used for N-acylation of diamine intermediate 13-69 (0.3 mmol, 0.2 g, 1 eq.) using TEA (435 μL, 5.0 eq, 3.13 mmol) and in 3 mL of Toluene at room temperature overnight to yield Lipid 21A. Crude product was purified 2× by column chromatography (Hexanes and EtAc, then DCM: 10% MeOH in DCM on silica) to obtain 51 mg of pure Lipid 21A (>99% by HPLC-UV and 95% by HPLC-CAD) and characterized by proton NMR and mass spectrometry (FIG. 4Y-1 and FIG. 4Y-2 for proton NMR and HPLC-CAD and Table 5 for Mass Spectrometry data).

### Synthesis of Lipid 22 and its Isomer

An isomer of lipid 22 was synthesized as provided in scheme 25-1 below. Briefly, alcohol 13-78 (accessed as described for Lipid 21 synthesis above) was used in the ring opening addition to cyclic anhydride 13-73′ to access intermediate 13-82. O-acylation of dihydroxyacetone with intermediate 13-82 using conditions described in Lipid 17 synthesis yielded ketone 13-83. Reductive amination of 13-83 with amine 15-3 using conditions described in the Lipid 17 synthesis yielded intermediate 13-84. Subsequent N-acylation of intermediate 13-84 with acid 13-31 using conditions analogous to those used for Lipid 9 synthesis afforded Lipid 22 isomer.

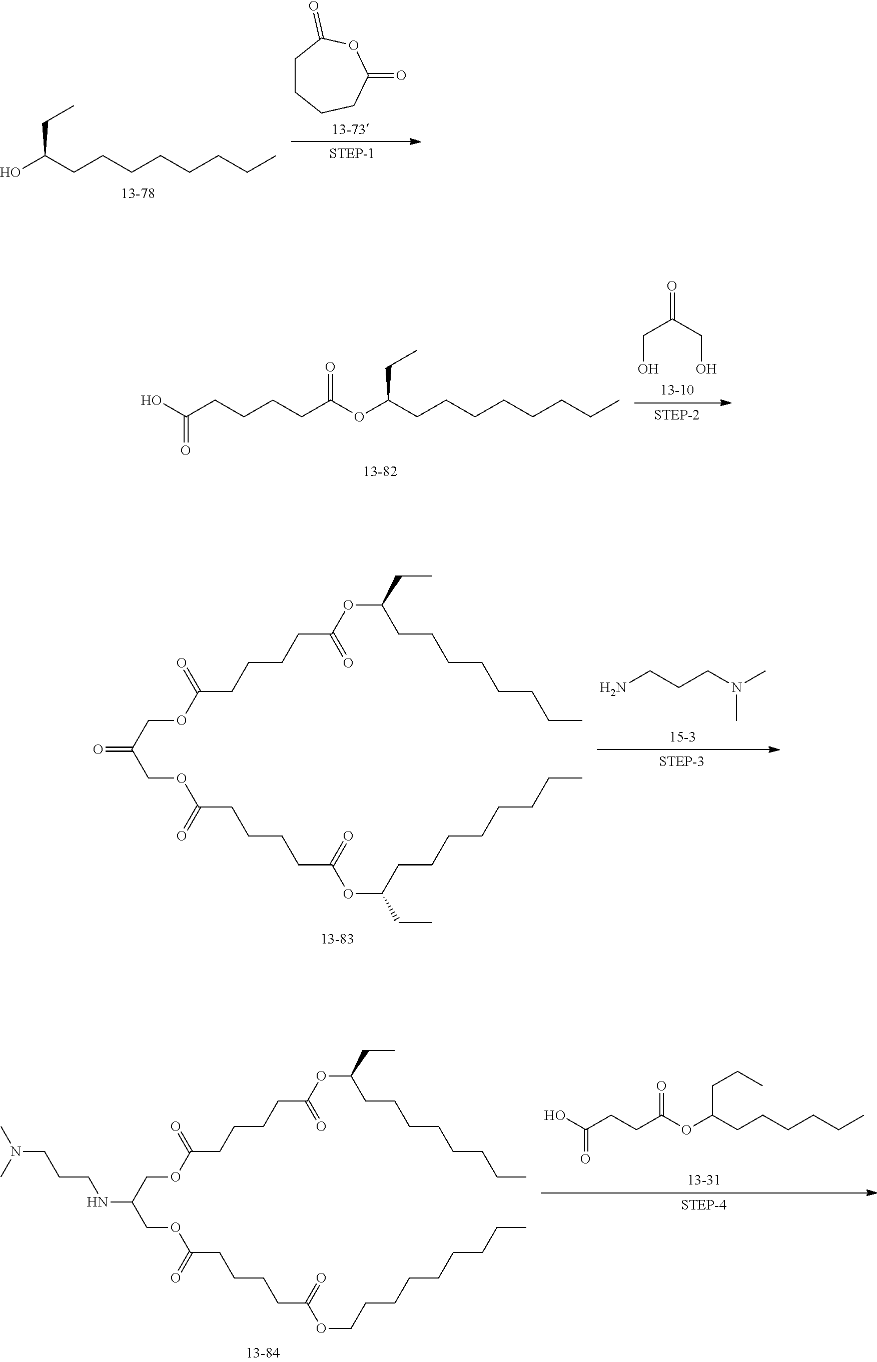

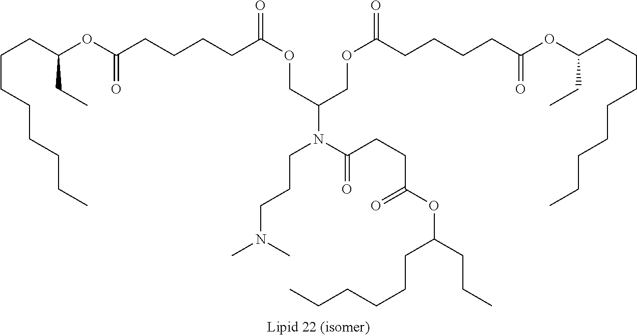 [SMILES: CCCCCCCC[C@H](CC)OC(=O)CCCCC(=O)OCC(COC(=O)CCCCC(=O)O[C@@H](CC)CCCCCCCC)N(CCCN(C)C)C(=O)CCC(=O)OC(CCC)CCCCCC]

Lipid 22 as a racemic mixture was synthesized as provided in scheme 25-2 below. Lipid 22 was accessed using methods described for Lipid 22 isomer above by replacing alcohol isomer 13-78 with racemic alcohol 13-78 rac.

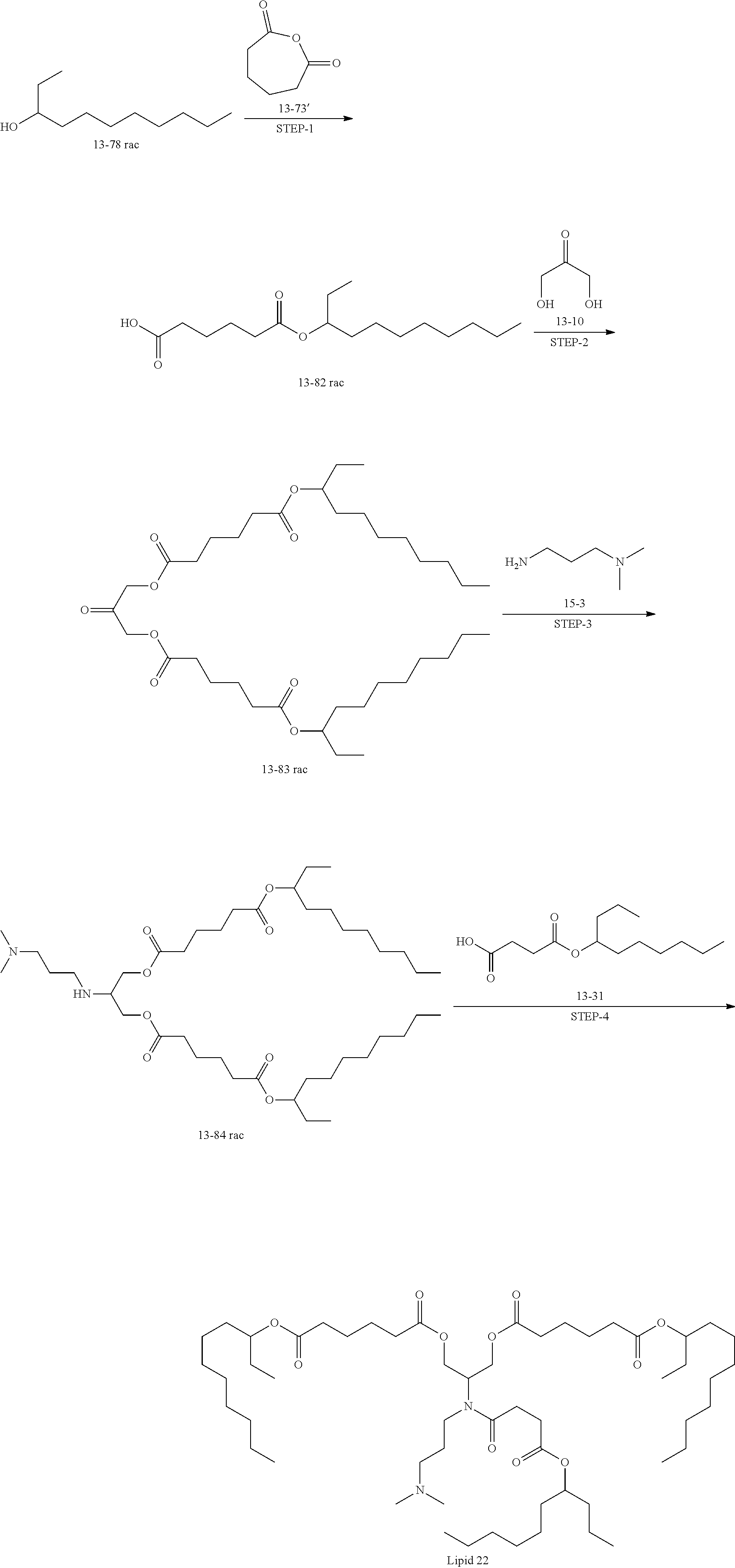

Alternatively, starting material 3-undecanol, 13-78-racemic (31.6 mmol, 5 g, 1 eq.) was acylated with adipic acid, 13-82 (9.8 mmol g, 1.68 g, 2.0 eq.) using EDCI (2.26 g, 1.2 eq, 11.8 mmol), DMAP (143 mg, 0.12 eq, 1.2 mmol) and TEA (6.8 mL, 5.0 eq, 49.0 mmol) in a mixture of 20 mL of DCM and 20 mL DMF at room temperature, overnight to obtain 1.35 g (46%) of acid intermediate, 13-83 rac. Starting material, 1,3-dihydroxyacetone, 13-10 (2.05 mmol, 184 mg, 1.0 eq.) was diacylated with acid Intermediate 13-83-rac (1.35 g, 2.2 eq, 4.5 mmol) using EDCI (856 mg, 2.2 eq, 4.5 mmol), DIPEA (782 μL, 2.2 eq, 4.5 mmol), and DMAP (127 mg, 0.5 eq, 1.03 mmol) in 6 mL of DCM at room temperature overnight to obtain 610 mg (46%) of ketone intermediate 13-84-rac.

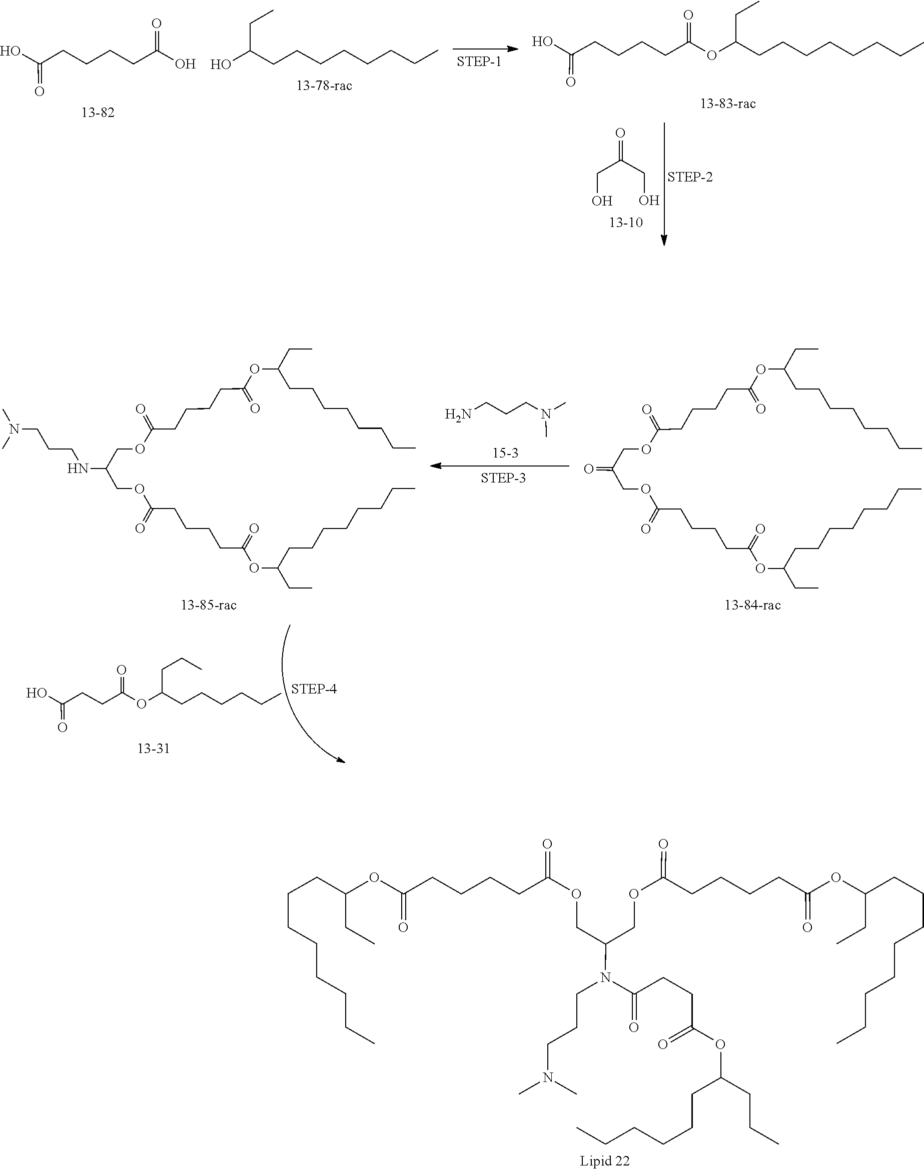

Intermediate 13-84-rac (0.93 mmol, 610 mg, 1.0 eq.) was converted to diamine intermediate 13-85-rac by reductive amination with N,N-dimethylamino-3-aminopropane, 15-3 (1.86 mmol, 233 μL, 2.0 eq.) using Na(OAc)₃BH (1.2 mmol, 249 mg, 1.2 eq.), acetic acid (1.86 mmol, 107 μL, 2.0 eq.) in 3 mL of DCM at room temperature for 3 hours. Purification of crude product by silica column chromatography (10% MeOH in DCM) yielded 210 mg of purified Intermediate 13-85-rac. Acid chloride 13-31′ (0.85 mmol, 233 mg, 3.0 eq.) from a previous batch was used for N-acylation of diamine intermediate 13-85-rac (0.28 mmol, 210 mg, 1 eq.) using TEA (1.4 mmol, 197 μL, 5.0 eq.) in 3 mL of Toluene at room temperature overnight to yield Lipid 22. Crude product was purified 2× by column chromatography (DCM: 10% MeOH in DCM) to obtain 56 mg (60%) of pure Lipid 22 (>99% by HPLC-CAD) and characterized by proton NMR and mass spectrometry (FIG. 4Z-1 and FIG. 4Z-2 for proton NMR and HPLC-CAD and Table 5 for Mass Spectrometry data A, B, C).

### Synthesis of Lipid 23

Lipid 23 was synthesized as provided in scheme 26 below. Briefly, O-acylation of dihydroxyacetone with acid 13-31 using conditions described in Lipid 9 synthesis yielded ketone 13-70. Reductive amination of 13-70 with amine 15-3 using conditions described in the Lipid 9 synthesis yielded intermediate 13-71. Subsequent N-acylation of intermediate 13-71 with acid 13-31 using conditions analogous to those used for Lipid 9 synthesis afforded Lipid 23.

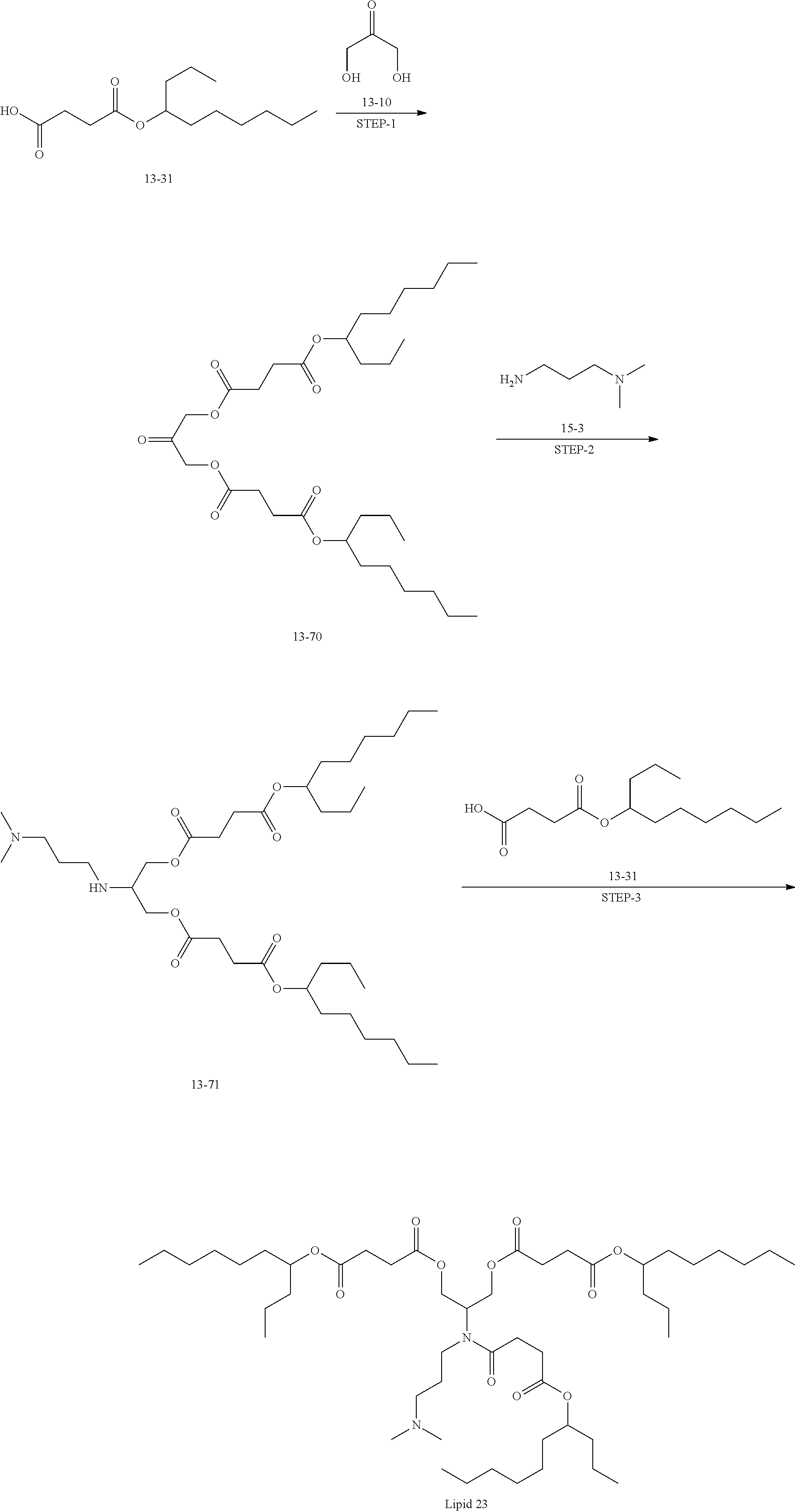

### Synthesis of Lipid 23A

Starting material 3-undecanol, 13-78-racemic (14.4 mmol, 2.47 g, 1 eq.) was acylated with suberic acid, 13-77 (5.0 g, 2.0 eq, 82.7 mmol) using EDCI (3.3 g, 1.2 eq, 17.3 mmol), DMAP (211 mg, 0.12 eq, 1.73 mmol) and TEA (10.0 mL, 5.0 eq, 72.0 mmol) in a mixture of 20 mL of DCM and 20 mL DMF at room temperature overnight to obtain 2 g (43%) of acid intermediate, 13-879-rac. Starting material, 1,3-dihydroxyacetone, 13-10 (2.8 mmol, 250 mg, 1.0 eq.) was diacylated with acid Intermediate 13-79-rac (2.0 g, 2.2 eq, 6.1 mmol) using EDCI (1.16 g, 2.2 eq, 6.1 mmol), DIPEA (1.06 mL, 2.2 eq, 6.1 mmol), and DMAP (172 mg, 0.5 eq, 1.4 mmol) in 8 mL of DCM at room temperature overnight to obtain 690 mg (35%) of ketone intermediate 13−80-rac.

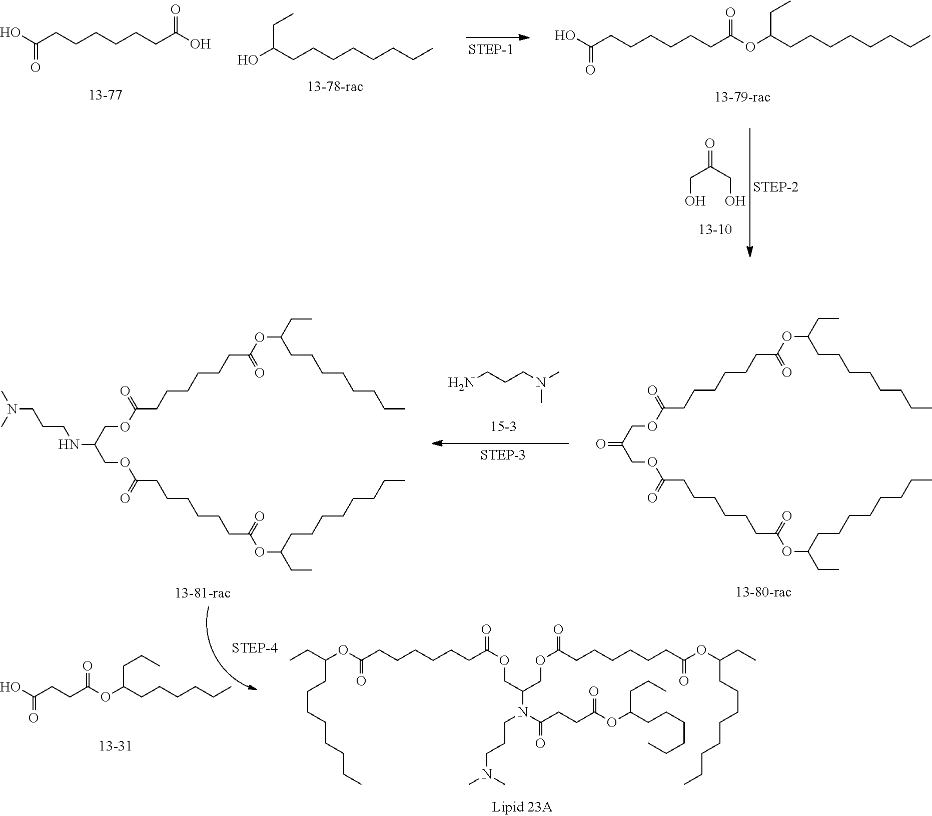

Intermediate 13-80-rac (0.97 mmol, 690 mg, 1.0 eq.) was converted to diamine intermediate 13-81-rac by reductive amination with N,N-dimethylamino-3-aminopropane, 15-3 (1.94 mmol, 243 μL, 2.0 eq.) using Na(OAc)₃BH (1.2 mmol, 249 mg, 1.2 eq.), acetic acid (1.94 mmol, 112 μL, 2.0 eq.) in 3 mL of DCM at room temperature for 3 hours. Purification of crude product by silica column chromatography (10% MeOH in DCM) yielded 520 mg of purified Intermediate 13-81-rac. Acid chloride 13-31′ (1.63 mmol, 447 mg, 2.5 eq.) from a previous batch was used for N-acylation of diamine intermediate 13-81-rac (0.65 mmol, 520 mg, 1 eq.) using TEA (3.25 mmol, 458 μL, 5.0 eq.) in 4 mL of toluene at room temperature overnight to yield Lipid 23A. Crude product was purified 2× by column chromatography (DCM: 10% MeOH in DCM) to obtain 230 mg (31%) of pure Lipid 23A (>99% by HPLC-UV and 96% by HPLC-CAD) and characterized by proton NMR and mass spectrometry (FIG. 4AA-1 and FIG. 4AA-2 for proton NMR and HPLC-CAD and Table 5 for Mass Spectrometry data).

### Synthesis of Lipid 24

Lipid 24 was synthesized as provided in scheme 27 below. Briefly, acid 13-34 was accessed by O-acylation of mono-protected di-acid 13-72 with alcohol 13-29 and subsequent deprotection of intermediate 13-73 to yield acid, 13-74. O-acylation of dihydroxyacetone with intermediate 13-74 using conditions described in Lipid 17 synthesis yielded ketone 13-75. Reductive amination of 13-75 with amine 15-3 using conditions described in the Lipid 17 synthesis yielded intermediate 13-76. Subsequent N-acylation of intermediate 13-76 with acid 13-31 using conditions analogous to those used for Lipid 9 synthesis afforded Lipid 24.

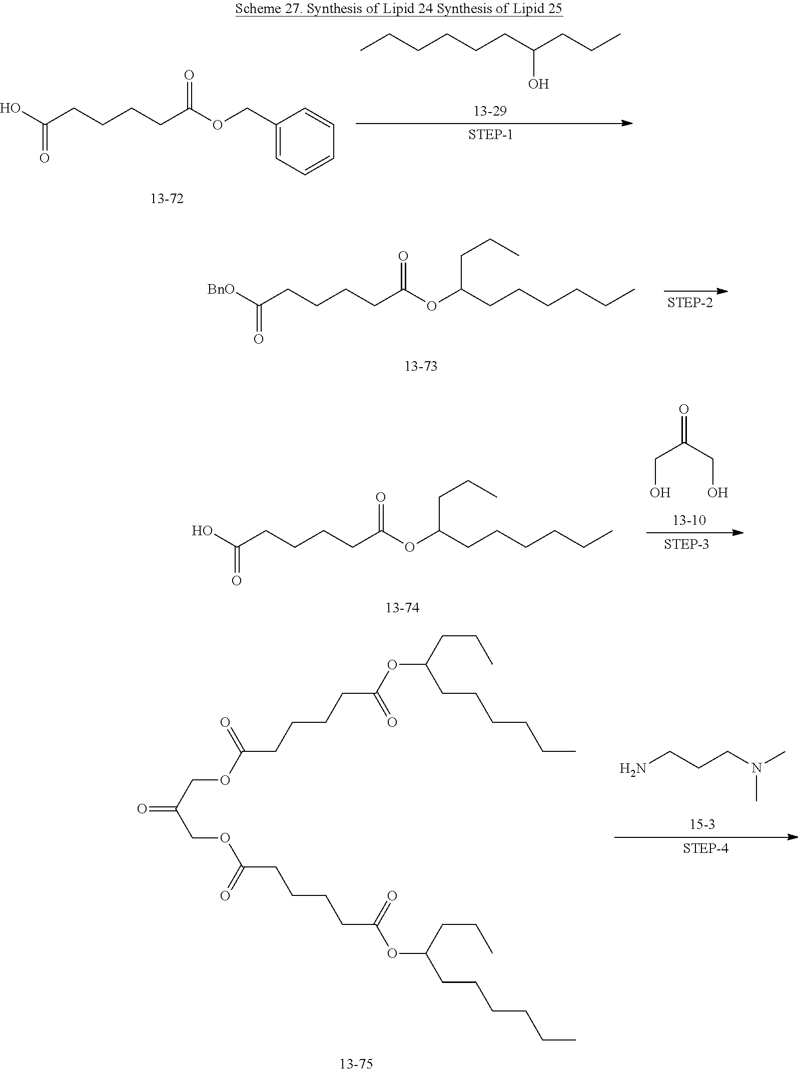

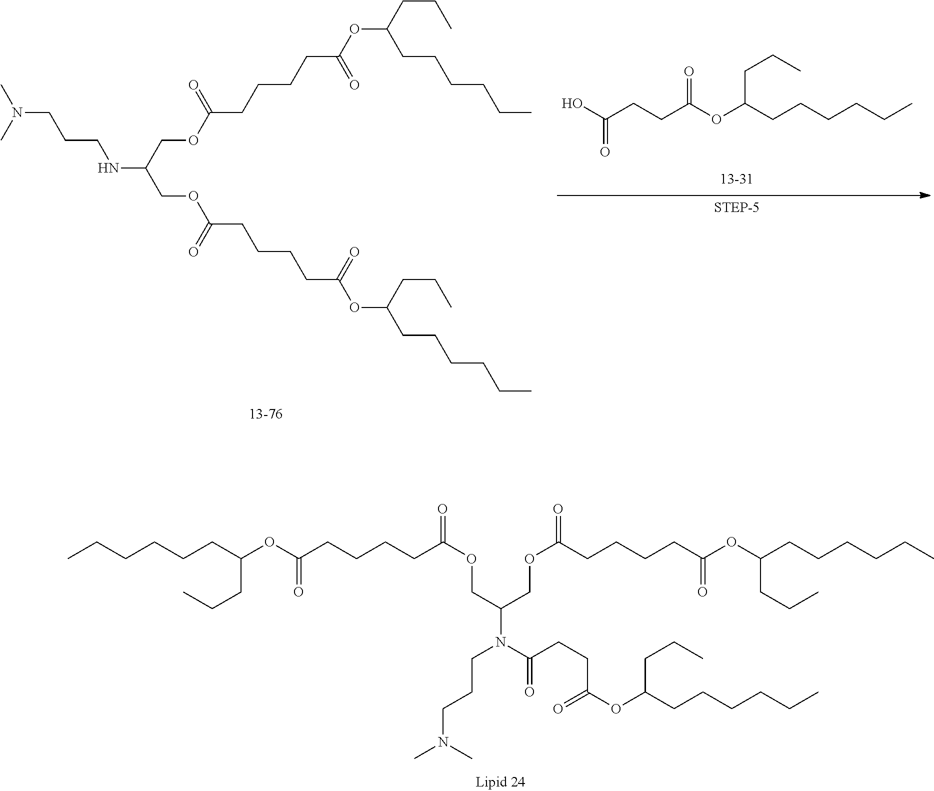 [SMILES: CCCCCCC(CCC)OC(=O)CCCCC(=O)OCC(COC(=O)CCCCC(=O)OC(CCC)CCCCCC)NCCCN(C)C ; CCCCCCC(CCC)OC(=O)CCC(=O)O ; CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

Lipid 25 was synthesized as provided in scheme 28 below. Briefly, ring opening addition of alcohol 13-29 to cyclic anhydride 13-52′ yielded acid intermediate 13-85. O-acylation of dihydroxyacetone with intermediate 13-85 using conditions described in Lipid 17 synthesis yielded ketone 13-86. Reductive amination of 13-86 with amine 15-3 using conditions described in the Lipid 17 synthesis yielded intermediate 13-87. Subsequent N-acylation of intermediate 13-87 with acid 13-31 using conditions analogous to those used for Lipid 9 synthesis afforded Lipid 25.

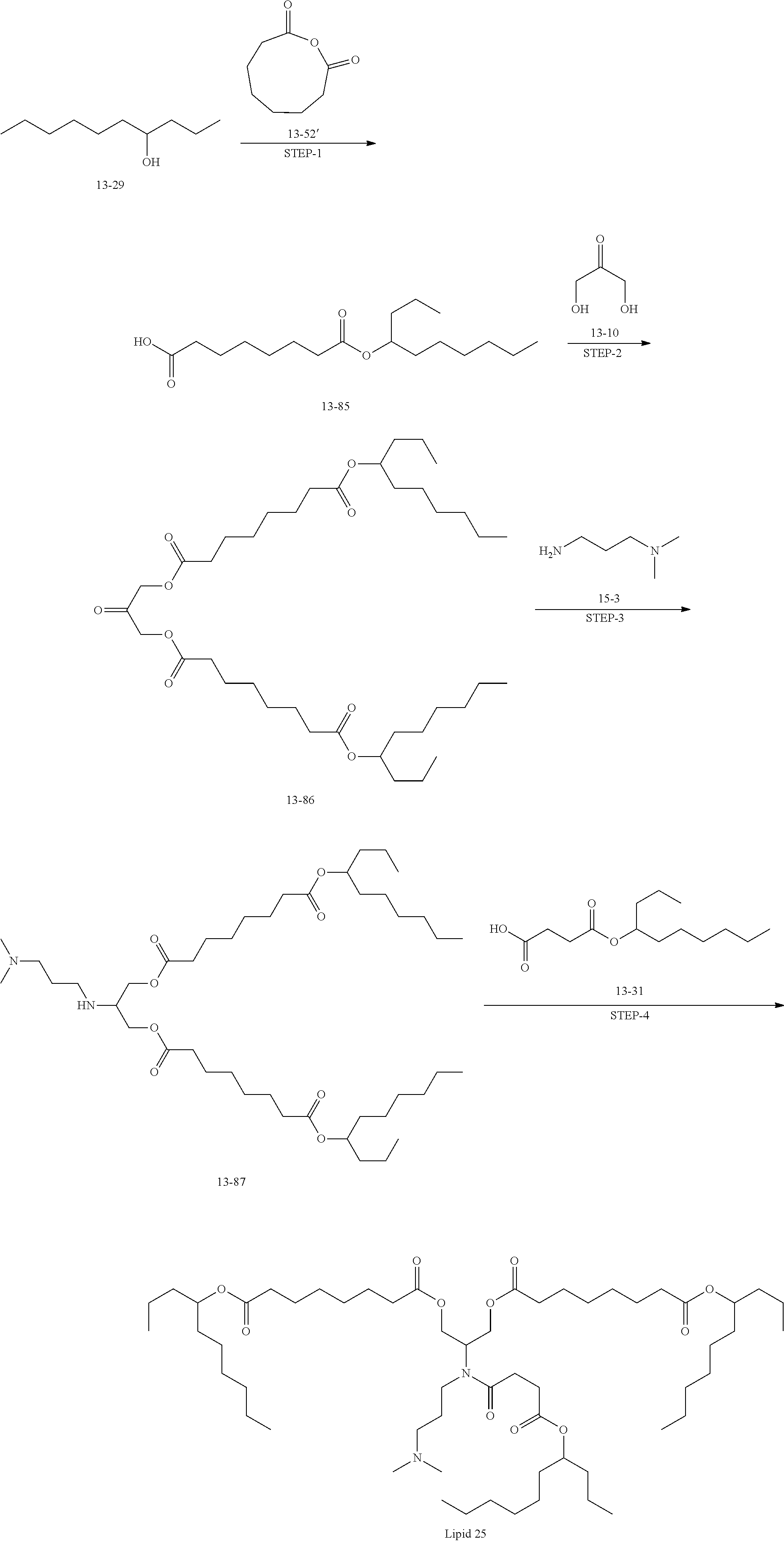

### Synthesis of Lipid 25A

Starting material benzyl protected glycolic acid 14-24 (15.4 mmol, 2.61 g, 1 eq.) was acylated with 2-hexyldecanoic acid 14-25 (6.10 g, 96%, 1.48 eq, 22.84 mmol) using EDCI (5.80 g, 1.97 eq, 30.26 mmol), DMAP (0.40 g, 0.21 eq, 3.27 mmol) and DIPEA (5.4 mL g, 1.97 eq, 30.33 mmol) in 60 mL of dichloromethane at room temperature overnight yielding 2.75 g (49%) of protected intermediate ester 14-26. Intermediate 14-26 (10.3 mmol, 4.1 g, 1.0 eq.) was deprotected using Pd/C (930 mg, 32% w/w), EtOAc (35 mL) at room temperature overnight yielding 1.72 g (82%) of acid intermediate 14-27.

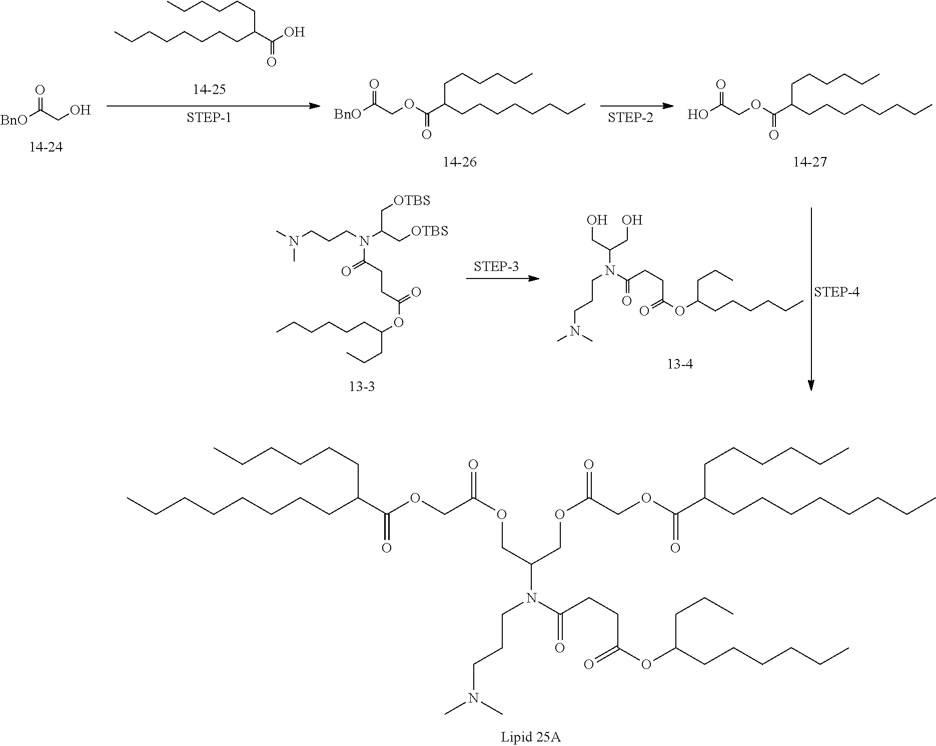

Protected intermediate 13-3 (600 mg, 0.9 mmol, 1 eq.) was treated with Hydrogen Fluoride/Pyridine (0.92 g, 10.0 eq, 9.3 mmol) in 4.0 mL THF at room temperature for 2 hours yielding dihydroxyl intermediate 13-4. Deprotection was confirmed by TLC and mass spectrometry. Acid intermediate 14-27 (5.4 mmol, 1.72 g, 1 eq.) was converted to the corresponding acid chloride 14-27′ using oxalyl chloride (2.36 g, 3.4 eq, 18.59 mmol) and DMF (100 μL, catalytic quantity) in 5.0 mL of Toluene at room temperature for 2 hours and conversion to chloride intermediate was confirmed by TLC.

Crude di-hydroxy intermediate 13-4 (350 mg, 0.84 mmol, 1 eq.) and crude acid chloride 14-27′ (1.58 g, 6.0 eq, 5.04 mmol) were combined with TEA (0.85 g, 10.0 eq, 8.4 mmol) in 9 mL of toluene at room temperature, overnight. Crude product was purified on ISCO column chromatography on silica column eluting with DCM and 10% MeOH in DCM yielding 240 mg (85%) of 99% pure (HPLC-CAD) Lipid 25A. (see FIG. 4AC-1 and FIG. 4AC-2 for characterization by proton NMR and LC-CAD purity and Table 5 for Mass Spectrometry data).

### Synthesis of Lipid 27

Lipid 27 was synthesized as provided in scheme 29 below and as follows. Ring opening of starting material caprolactone 14-30 (2 g, 17.5 mmol) using 5 mL of 0.5 M NaOH at room temperature for 2 hours yielded 6-hydroxyhexanoic acid 14-31 (1.8 g, 78%). Additional 14-31 was produced in a second 2 g scale caprolactone hydrolysis reaction using identical conditions to obtain 1.9 g (82%) of 14-31. 6-hydroxyhexanoic acid 14-31 (1 g, 7.5 mmol) was benzylprotected using DBU (1.38 g, 1.2 eq, 9 mmol), Benzylbromide (1.68 g, 1.3 eq, 9.8 mmol) in 6 mL of MeOH and 10 mL of DMF at 0° C. to room temperature, overnight yielding protected intermediate 14-32 (1.3 g, 77%). Additional 14-31 (2 g, 15.1 mmol) was protected using DBU (2.76 g, 1.2 eq, 18.1 mmol), Benzylbromide (3.36 g, 1.3 eq, 19.6 mmol) in 12 mL of MeOH and 20 mL of DMF at 0° C. to room temperature, overnight yielding protected intermediate 14-32 (2.65 g, 79%).

Intermediate 14-32 (2.45 g, 1 eq, 11 mmol) was used to acylate acid 14-25 (2.59 g, 1.5 eq, 10 mmol) using EDCI (2.58 g, 2 eq, 13.4 mmol), DIPEA (1.74 g, 2 eq, 13.4 mmol) and DMAP (0.16 g, 0.2 eq, 1.3 mmol) in 20.0 mL DCM at room temperature overnight. Crude product was purified by column chromatography (1×) yielding 4.8 g (94%) of pure protected intermediate 14-33. Subsequent deprotection of 14-33 (4.8 g, 22 mmol) by hydrogenation on Pd/C/Hydrogen gas (0.6 g, 20% w/w) in 30 mL of ethylacetate yielded 3.68 g (95%) of free acid 14-34 of column purified material.

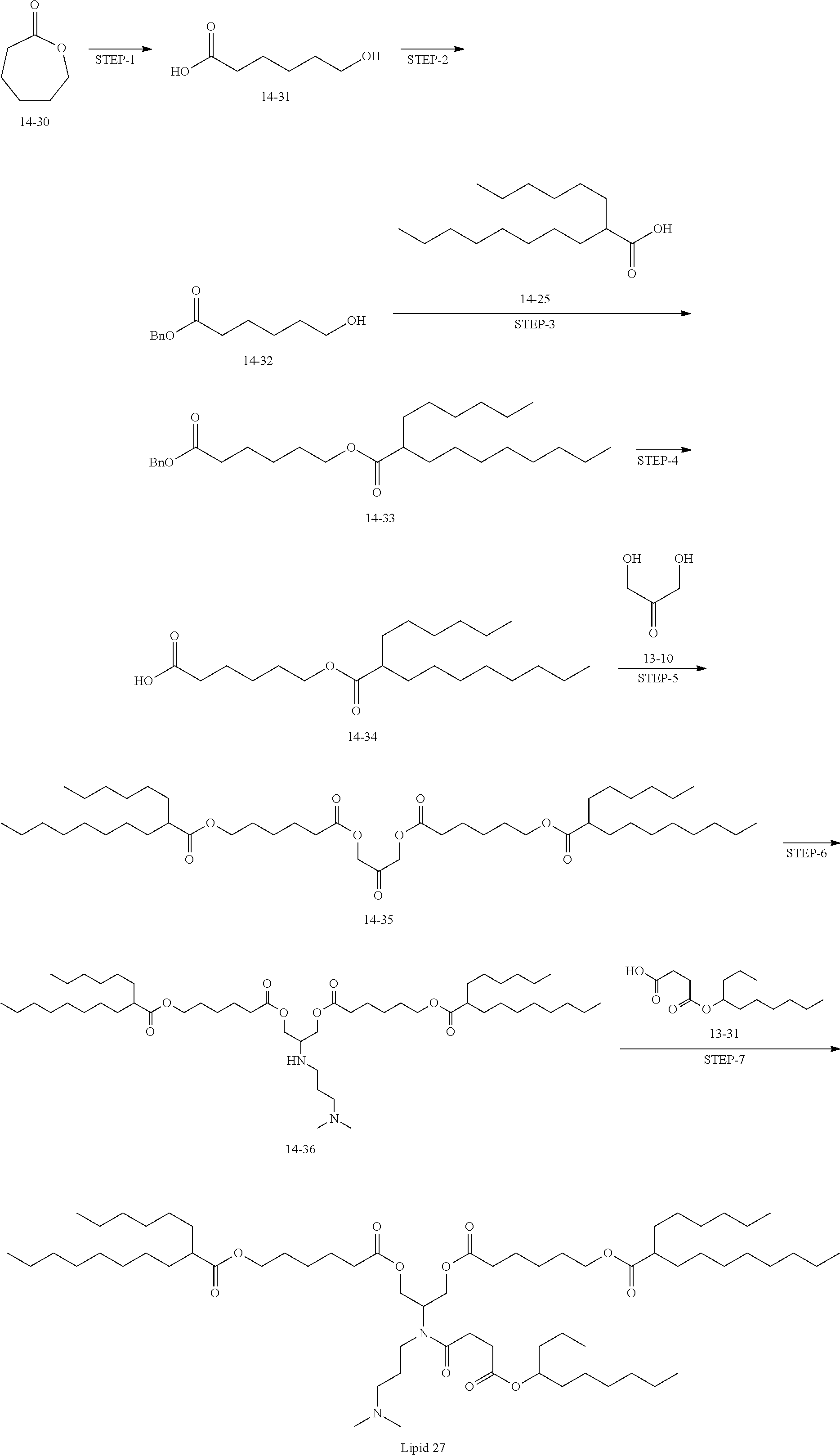

Acid intermediate 14-34 (1.74 g, 2.5 eq, 5.5 mmol) was used to acylate dihydroxyacetone, 13-10 (200 mg, 1.0 eq, 6 mmol), EDCI (1.06 g, 2.5 eq, 5.5 mmol), DIPEA (0.71 g, 2.5 eq, 5.5 mmol), and DMAP (54 mg, 0.2 eq, 0.4 mmol) in 10.0 mL DCM at room temperature, overnight to obtain ketone 14-35. Crude product was purified by column chromatography to obtain 1.27 mg (76%) of pure 14-35. Reductive amination of 14-35 (1.26 mg, 1.0 eq, 1.5 mmol) with amine 15-3 (0.32 g, 2.0 eq, 3.1 mmol) using acetic acid (0.19 g, 2.0 eq, 3.1 mmol) and Na(OAc)₃BH (0.50 g, 1.5 eq, 2.3 mmol) in 15 mL DCM at room temperature of 3 hours yielded intermediate 14-36 (330 mg, 34%).

Compound 13-31 (290 mg, 1.1 mmol) from a previous batch was converted to the corresponding acid chloride 13-31′ using oxalyl chloride (0.48 g, 3.4 eq, 3.8 mmol) and DMF (20 μL, catalytic quantity) in 4 mL of Toluene for 2 hours at room temperature. Crude 13-31′ (0.29 g, 3.0 eq, 1.1 mmol) was used for N-acylation of 14-36 (330 mg, 1.0 eq, 0.3 mmol) using TEA (0.26 mL, 5.0 eq, 1.8 mmol) 5 mL toluene at room temperature, overnight to obtain column purified Lipid 20 (180 mg, 43%) of >98% purity (HPLC-CAD).

See FIG. 4AE-1 and FIG. 4AE-2 for Lipid 27 NMR spectrum, and reverse phase LC-CAD chromatogram, and Table 5 for product mass.

### Synthesis of Lipid 28

Lipid 28 was synthesized as provided in scheme 30 below and as follows. Intermediate 14-36 was produced as described above (see Synthesis of Lipid 27).

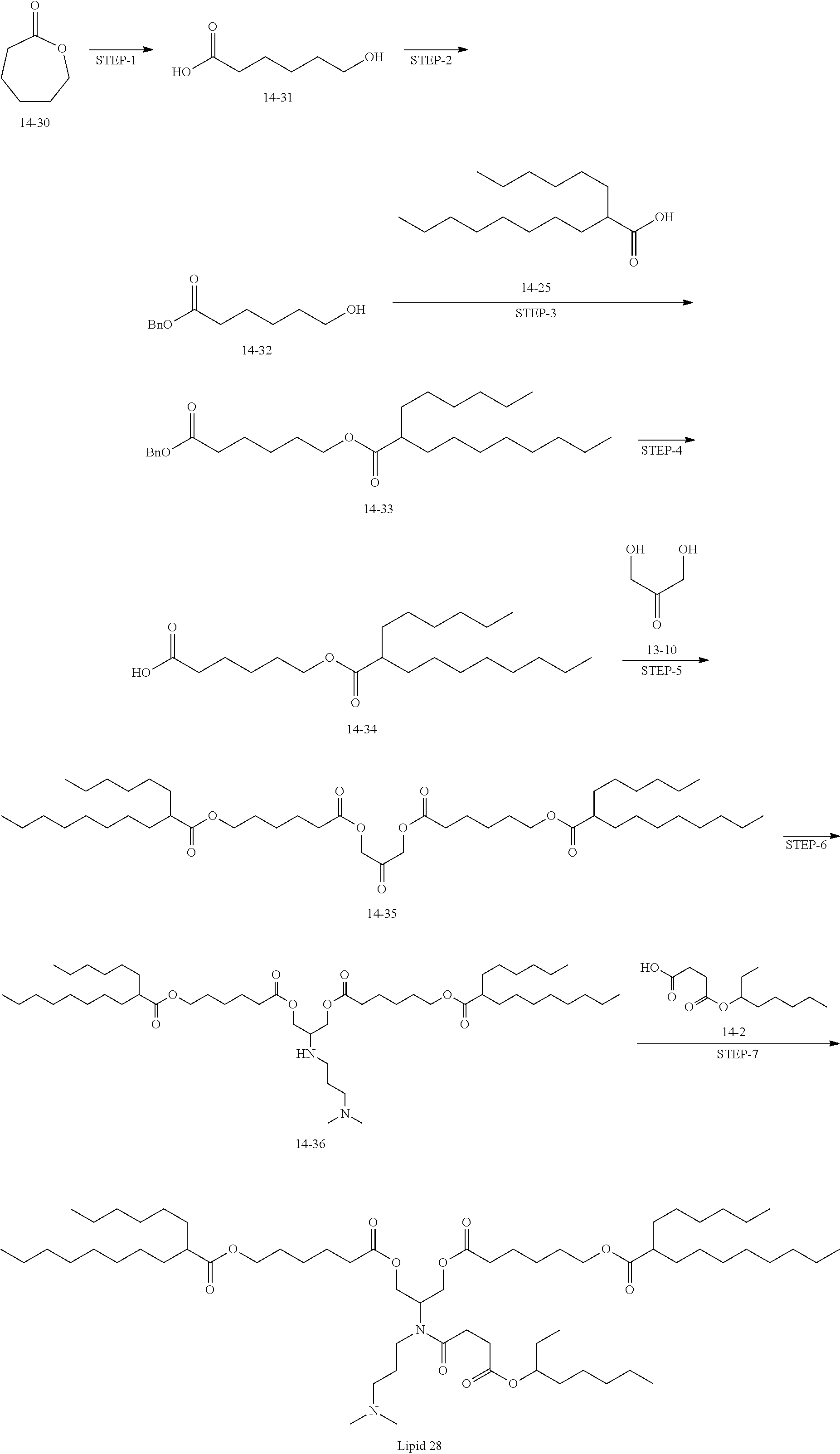

Acid intermediate 14-2 was produced by acylation of starting material 3-hydroxyoctanol 14-1 with succinic acid using 13-30 (1.53 g, 2.0 eq, 15.3 mmol) and DMAP (0.93 g, 1.0 eq, 7.6 mmol) and 2 mL of Pyridine in a mixture of 2 mL of THF and 5 mL of DCM to obtain 1.1 g (64%) of 14-2. 14-2 (348 mg, 1.51 mmol) was converted to the corresponding acid chloride 14-2″ using oxalyl chloride (0.651 g, 5.13 mmol, 3.4 eq) and DMF (40 μL, catalytic quantity) in 3 mL of Toluene for 2 hours at room temperature. Crude 14-2′ (0.348 g, 1.51 mmol, 3.1 eq.) was used for N-acylation of 14-36 (430 mg, 1.51) using TEA (0.41 mL, 2.94 mmol, 6.02 eq) in 6 mL toluene and 2.5 mL DCM at room temperature, overnight to obtain column purified (eluting with 10% methanol in DCM) Lipid 20 (148 mg, 28%) of 85% purity (HPLC-CAD). Second column purification (eluting with 5% methanol in DCM) yielded 115 mg of 94% purity (HPLC-CAD).

See FIG. 4AF-1 for Lipid 27 NMR spectrum, FIG. x4AF-2 for Lipid 28 reverse phase LC-CAD chromatogram, and Table 5 for product mass.

### Synthesis of Lipid 29

Benzyl protected malic acid 14-4 (14.1 mmol, 3.18 g, 1 eq.) was acylated with N-decanoic acid 14-12 (3.86 g, 1.5 eq, 21.2 mmol) using HATU (8.1 g, 1.5 eq, 21.2 mmol), DBU (4.3 g, 2.0 eq, 28.3 mmol) in 30 mL of DMF at room temperature overnight yielding 1.9 g (36%) of protected intermediate ester 14-13. Intermediate 14-13 (5.2 mmol, 1.9 g, 1 eq.) was acylated with hexanoyl chloride 14-6, (2.8 g, 4.0 eq, 20.8 mmol), TEA (2.63 g, 5.0 eq, 26.0 mmol), DMAP (127 mg, 0.2 eq, 1.0 mmol) in 20 mL of Toluene at room temperature, overnight to obtain intermediate 14-14 (630 mg, 26%). Intermediate 14-14 (2.8 mmol, 1.3 g, 1.0 eq.) was deprotected using Pd/C (260 mg, 32% w/w) in 15 mL Ethyl Acetate at room temperature overnight yielding 1.025 g (98%) of acid intermediate 14-15.

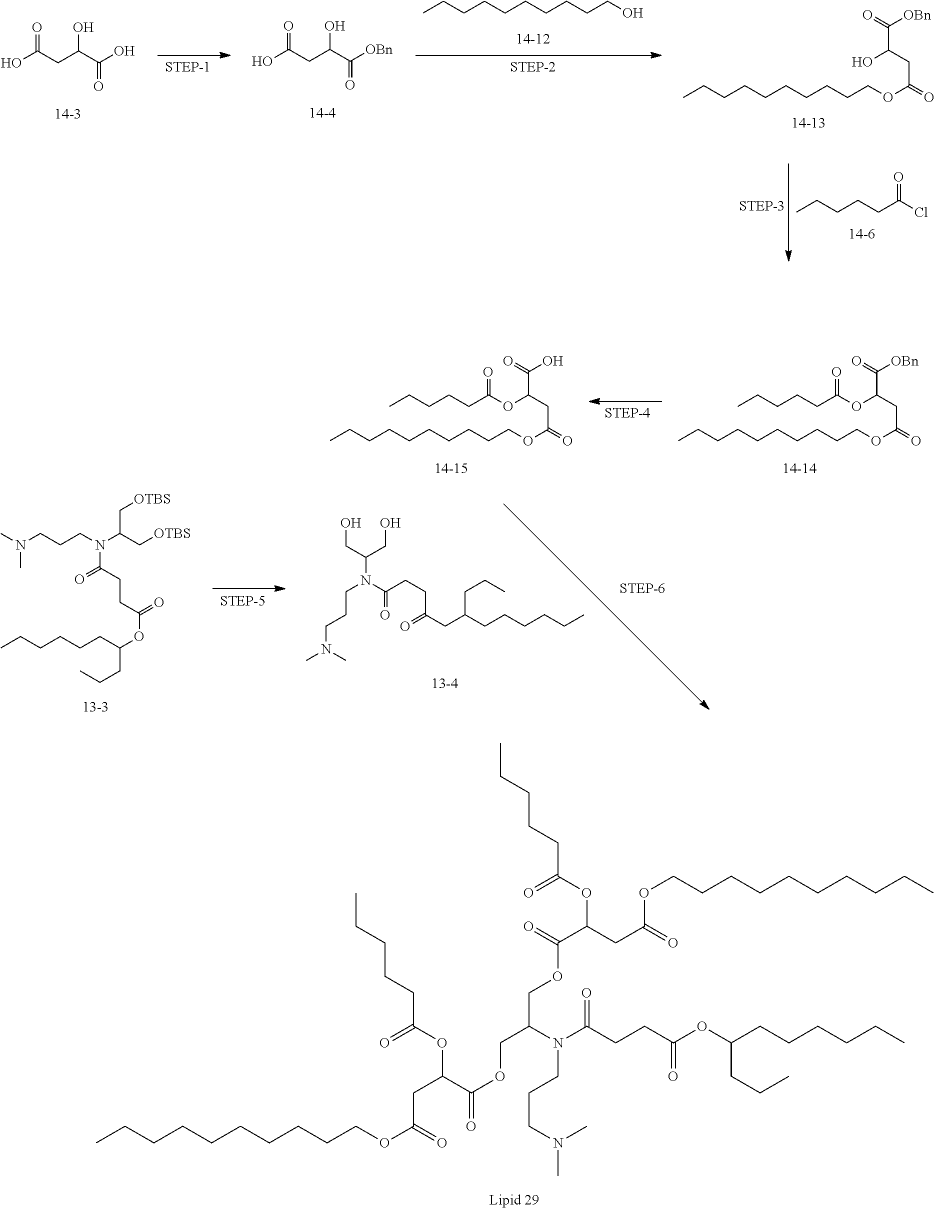

Acid intermediate 14-15 (2.6 mmol, 1.0 g, 1 eq.) was converted to the corresponding acid chloride 14-15′ using oxalyl chloride (0.82 mL, 3.4 eq, 9.1) and DMF (100 μL, catalytic quantity) in 6.0 mL of Toluene at room temperature for 2 hours and conversion to chloride intermediate was confirmed by TLC.

Protected intermediate 13-3 (0.72 mmol, 300 mg) using HF-pyridine, (0.71 mL, 10.0 eq, 7.2 mmol) in 4.0 mL THF at room temperature, overnight and crude di-hydroxy intermediate 13-4 (195 mg, 0.46 mmol, 1 eq.) and crude acid chloride 14-15′ (1.097 g, 6.0 eq, 2.7 mmol) were combined with TEA (0.64 mL, 10.0 eq, 4.6 mmol) in 5 mL of toluene at room temperature, overnight. Crude product was purified on ISCO column chromatography on silica column eluting with DCM and 10% MeOH in DCM yielding to obtain 270 mg (51%) of >99% pure (HPLC-CAD)

Lipid 29. (see FIG. 4AG-1 and FIG. 4AG-2 for characterization by proton NMR and LC-CAD purity, and Table 5 for Mass Spectrometry data).

### Synthesis of Lipid 31

Lipid 31 was synthesized as provided in scheme 32 below and as follows. Starting material 15-1 (68 mmol, 10 g, 1 eq.) was treated with p-toluene sulfonyl chloride (70 mmol, 13.3 g, 1.03 eq.) using Pyridine (80 mmol, 10.1 ml, 1.2 eq.) in 150 mL DCM to obtain protected intermediate 15-2. Crude product was recrystallized in ethyl acetate and hexane yielding 20.4 g (99%) of pure intermediate 15.2. Intermediate 15-4 was accessed by reaction of 15-2 (16.5 mmol, 5 g, 1.2 eq.) and diamine 15-3 (33 mmol, 3.35 g, 2 eq.) in 40 mL dioxane under reflux conditions. Crude product was purified by column chromatography to obtain 3.5 g (91%) of pure intermediate 15-4. N-acylation of 15-4 (108 mg, 0.268 mmol) using nonanoic acid 13-12 (0.67 mmol, 106 mg, 2.5 eq) using EDCI (0.67 mmol, 128 mg, 2.5 eq.) and DIEA (0.67 mmol, 86 mg, 2.5 eq) and DMAP (3 mg) in 10 mL DCM yielded amine 15-5. Crude product was purified by column chromatography to obtain 113 mg (65%) of purified diamine 15-5. Diol intermediate 15-6 was accessed by deprotection of 15-5 (113 mg) in 4 mL of 1M HCl and THF (1:3 v/v) at room temperature for 8 hours in quantitative yield (102 mg). Intermediate 15-6 (0.3 mmol, 100 mg, 1 eq) was acylated with linoleic acid 1-5 (0.9 mmol, 250 mg, 3 eq) using EDCI (0.9 mmol, 172 mg, 3 eq), DIPEA (0.9 mmol, 116 mg) and DMAP (10 mg, catalytic quantity) to obtain Lipid 31. Crude product was purified by column chromatography yielding 120 mg (46%) of pure Lipid 31 (>99% purity by LC-ELSD) and characterized by proton NMR and Mass Spectrometry (see FIG. 4R-1 for Lipid 31 NMR spectrum, FIG. 4R-2 for Lipid 31 LC-MS, and Table 5 for product mass).

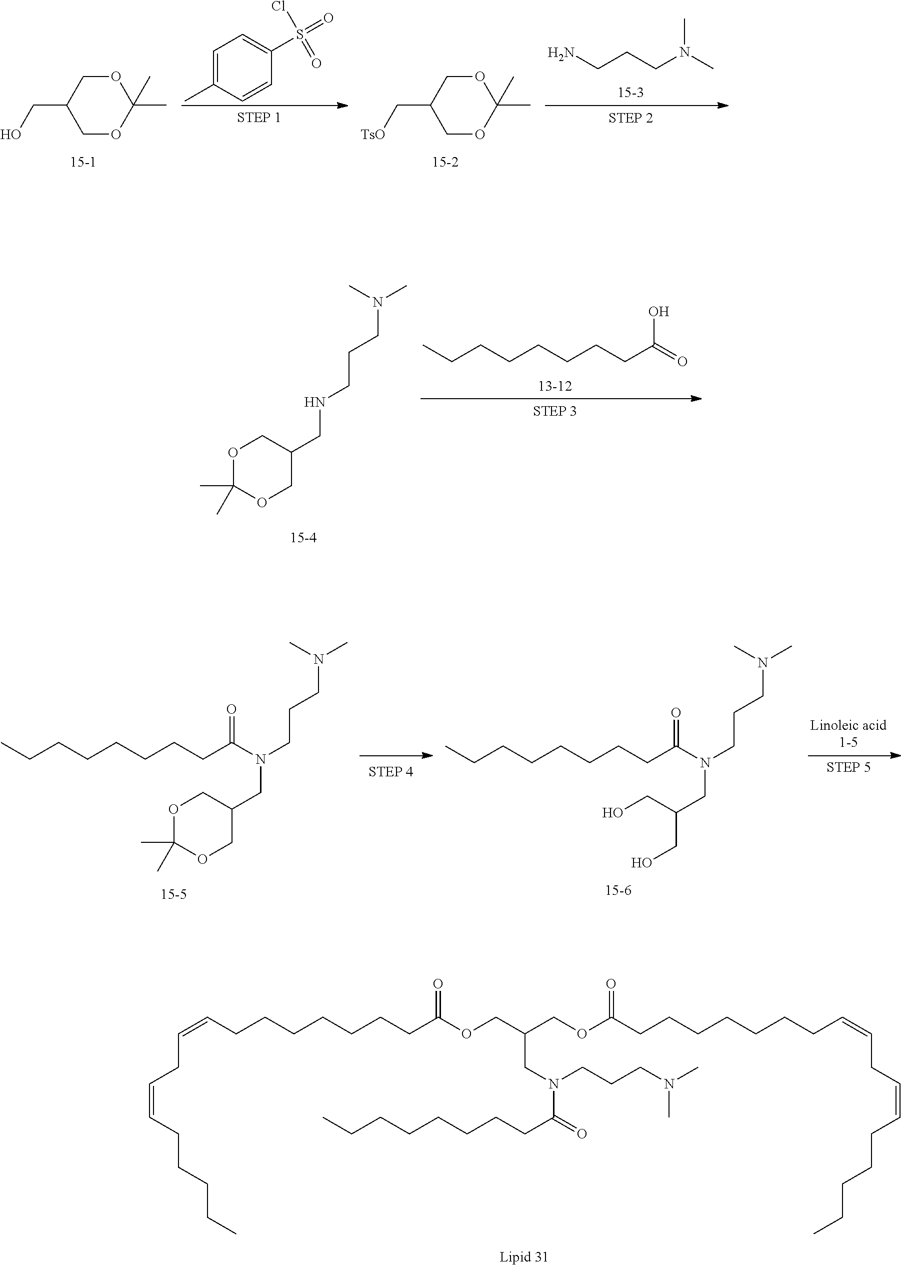

### Synthesis of Lipid 32

Lipid 32 was synthesized as provided in scheme 33 below and as follows. Intermediate 15-4 was produced as described for Lipid 31 above (steps 1 and 2, Scheme 30). N-acylation of 15-4 (4.34 mmol, 1 g, 1.0 eq) using 2-ethyl heptanoic acid 13-13 (10.85 mmol, 1.71 g, 2.5 eq) using EDCI (10.85 mmol, 2.07 g, 2.5 eq), DIEA (10.85 mmol, 1.40 g, 2.5 eq) and DMAP (10 mg) in 100 mL DCM yielded amine 15-7. Crude product was purified by column chromatography to obtain 724 mg (52%) of purified diamine 15-7. Diol intermediate 15-8 was accessed by deprotection of 15-7 (714 mg) in 3 mL of 1M HCl and 7 mL THF at room temperature for 1 hour in quantitative yield. Intermediate 15-8 (1.9 mmol, 630 mg, 1 eq) was acylated with linoleic acid 1-5 (6.49 mmol, 1.82 g, 3.4 eq) using EDCI (6.49 mmol, 1.23 g, 3.4 eq), DIPEA (6.49 mmol, 830 mg, 3.4 eq) and DMAP (20 mg, catalytic quantity) to obtain Lipid 32. Crude product was purified by column chromatography (4×) yielding 27 mg of pure fraction (>98% purity by LC-ELSD) of Lipid 32 and characterized by proton NMR and Mass Spectrometry (see FIG. 4S-1 for Lipid 32 NMR spectrum, FIG. 4S-2 for Lipid 32 LC-MS, and Table 5 for product mass).

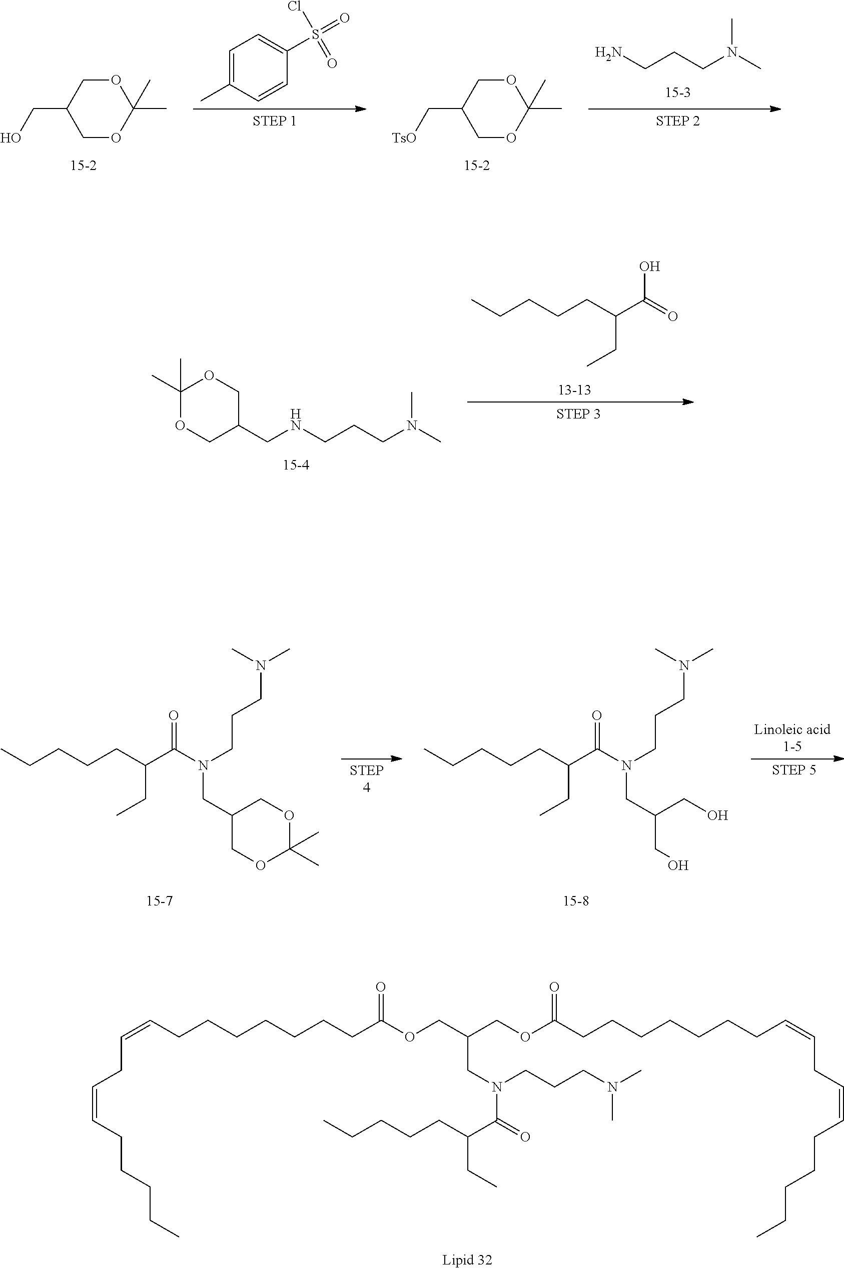

### Synthesis of Lipid 33

Lipid 33 was synthesized as provided in scheme 34 below and as follows. Starting material 15-1 (34.3 mmol, 5 g, 1 eq.) was tosylated using p-toluene sulfonyl chloride, TsCl (6.52 g, 1 eq, 34.3 mmol) using TEA (19.01 mL, 4 eq, 137 mmol) and DMAP (30 mg) in 200 mL DCM. Crude product was purified by column chromatography (1×) to obtain 10.2 g (98%) of reactive intermediate 15-2. Nucleophilic displacement of 15-2 (10.0 mmol, 2.3 g, 1 eq.) with diamine 15-9 (9.24 mmol, 1.0 mL, 1.2 eq.) in 10 mL dioxane (10 mL) yielded 1.6 g (97%) of compound 15-10. Nucleophilic displacement reaction was repeated using additional 15-2 (8.3 mmol, 2.5 g, 1 eq.) and diamine 15-9 (9.9 mmol, 1.1 mL, 1.2 eq.) in 50 mL dioxane for an additional quantity of compound 15-10. Crude products from the two reactions were purified by column chromatography to obtain a total of 1.7 g (~50%) pure 15-10. N-acylation of 15-10 (4.05 mmol, 875 mg, 1 eq.) with nonanoic acid 13-12 (7.1 mmol, 1.24 mL, 1.8 eq.) using EDCI (1.4 g, 1.8 eq, 7.1 mmol), DIPEA (1.3 mL, 1.8 eq, 7.1 mmol) and DMAP (90 mg, 0.2 eq, 0.81 mmol) in 8 mL DCM yielded intermediate 15-11. Crude product was purified by column chromatography (2×) to obtain 230 mg (16%) of pure intermediate 15-11. Deprotection of 15-11 (0.64 mmol, 230 mg, 1 eq.) in 5 mL of 4M HCl in dioxane yielded intermediate 15-12. Crude product was purified by column chromatography (1×) to obtain 74 mg (37%) of pure intermediate 15-12. Intermediate 15-12 (0.24 mmol, 74 mg, 1 eq.) was acylated with linoleic acid 1-5 (169 mg, 2.5 eq, 0.58 mmol) using EDCI (120 mg, 2.5 eq, 0.58 mmol), DIPEA (102 μL, 2.5 eq, 0.58 mmol) and DMAP (6 mg, 0.2 eq, 0.048 mmol) in 5 mL DCM to obtain Lipid 33. Crude product was purified by column chromatography (2×) to obtain 64 mg (32%) of pure Lipid 33 (>99% purity by LC-ELSD) and characterized by proton NMR and Mass Spectrometry (see FIG. 4T-1 for Lipid 33 NMR spectrum, FIG. 4T-2 for Lipid 33 LC-MS, and Table 5 for product mass).

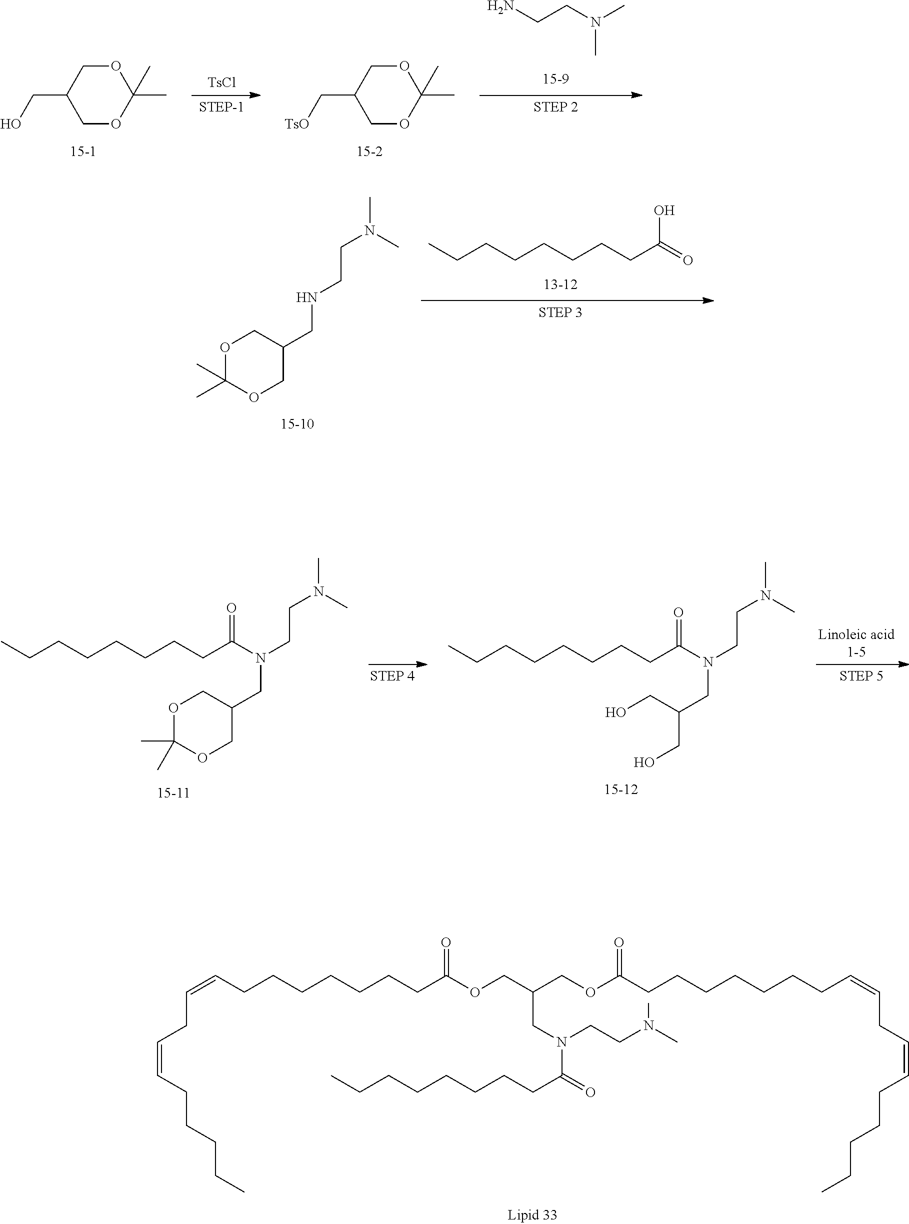

### Synthesis of Lipid 34

Lipid 34 was synthesized as provided in scheme 35 below and as follows. Intermediate 15-2 was accessed as described for Lipid 33. Nucleophilic displacement of 15-2 (3.3 mmol, 1 g, 1 eq.) with diamine 15-13 (3.9 mmol, 0.46 mL, 1.2 eq.) in 6 mL dioxane (10 mL) yielded 520 mg (64%) of compound 15-14. Reaction was repeated to access an additional 400 mg of pure compound 15-14. N-acylation of 15-14 (2.6 mmol, 620 mg, 1 eq.) with nonanoic acid 13-12 (5.3 mmol, 915 μL, 2.0 eq.) using EDCI (5.3 mmol, 1.06 g, 2.0 eq.), DIPEA (923 μL, 2.0 eq, 5.3 mmol) and DMAP (58 mg, 0.2 eq, 0.05 mmol) in 10 mL DCM yielded intermediate 15-15. Crude product was purified by column chromatography (2×) to obtain 355 mg (35%) of pure intermediate 15-15. Deprotection of 15-15 (1.03 mmol, 355 mg, 1 eq.) in 7 mL of 4M HCl in dioxane yielded intermediate 15-16. Crude product was purified by column chromatography (2×) to obtain 40 mg (13%) of pure intermediate 15-16. Intermediate 15-16 (0.116 mmol, 40 mg, 1 eq.) was acylated with linoleic acid 1-5 (81 mg, 2.5 eq, 0.29 mmol) using EDCI (55 mg, 2.5 eq, 0.29 mmol), DIPEA (3.2 μL, 2.5 eq, 0.29 mmol) and DMAP (2 mg, 0.2 eq, 0.05 mmol) in 10 mL DCM to obtain Lipid 34. Crude product was purified by column chromatography (2×) to obtain 73 mg (73%) of pure Lipid 34 (>99% purity by LC-ELSD) and characterized by proton NMR and Mass Spectrometry (see FIG. 4U-1 for Lipid 34 NMR spectrum, FIG. 4U-2 for Lipid 34 LC-MS, and Table 5 for product mass).

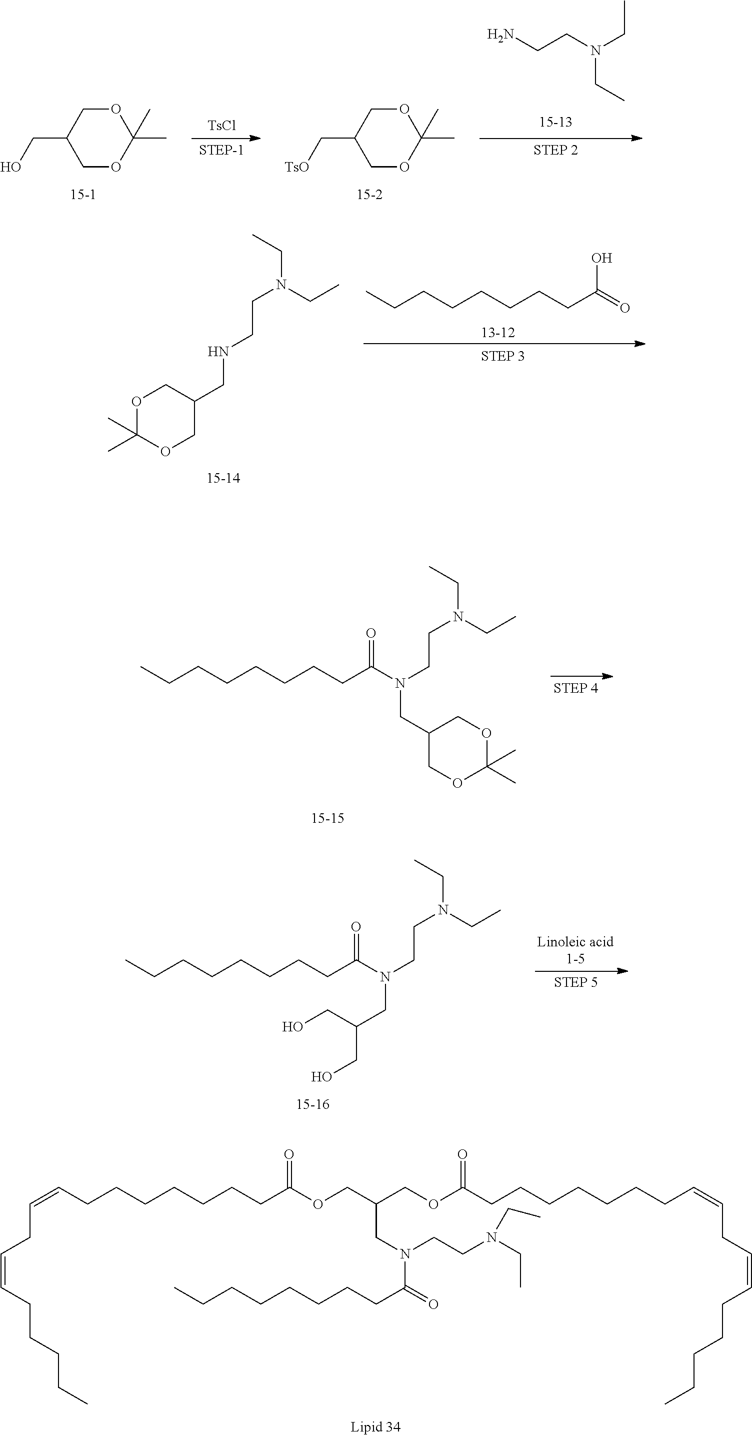

### Synthesis of Lipid 35

Lipid 35 was synthesized as provided in scheme 36 below.

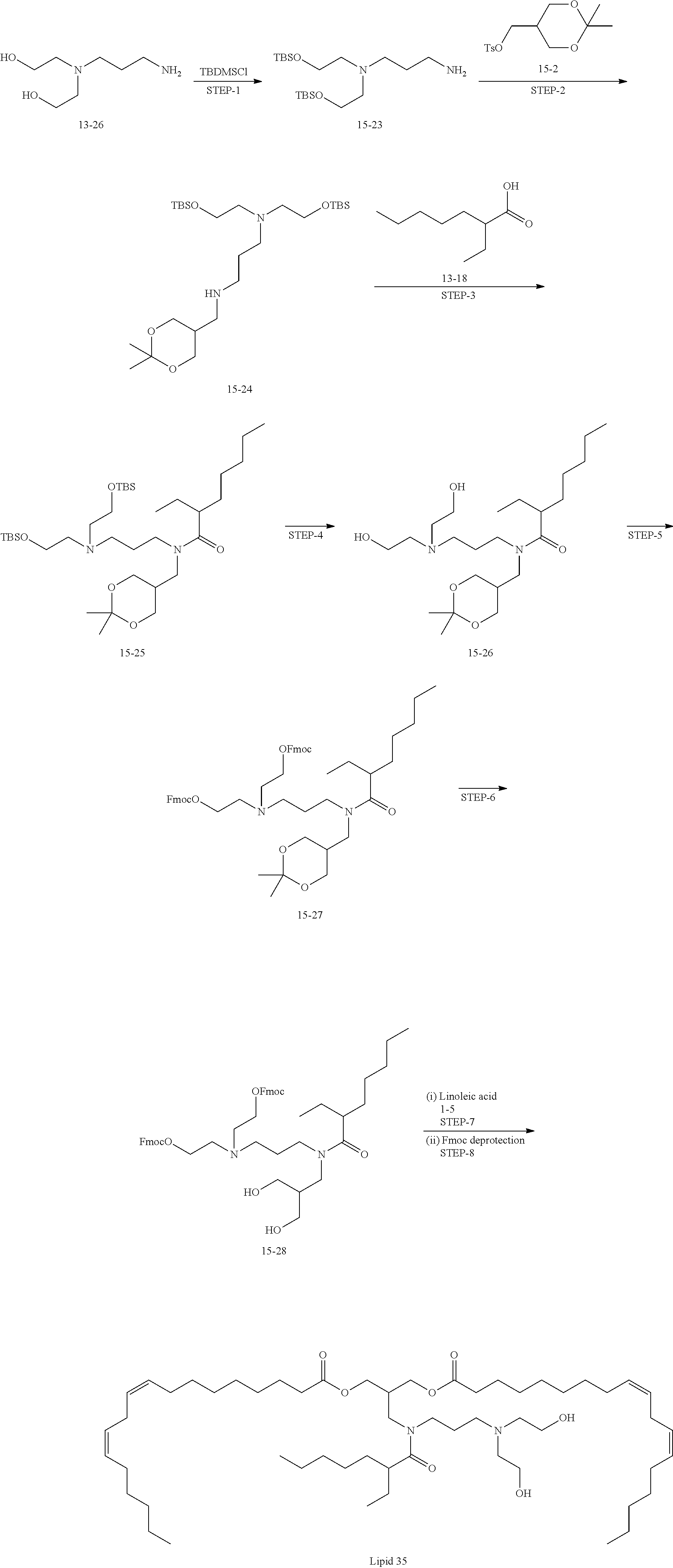

### Synthesis of Lipid 36

Lipid 36 was synthesized as provided in scheme 37 below.

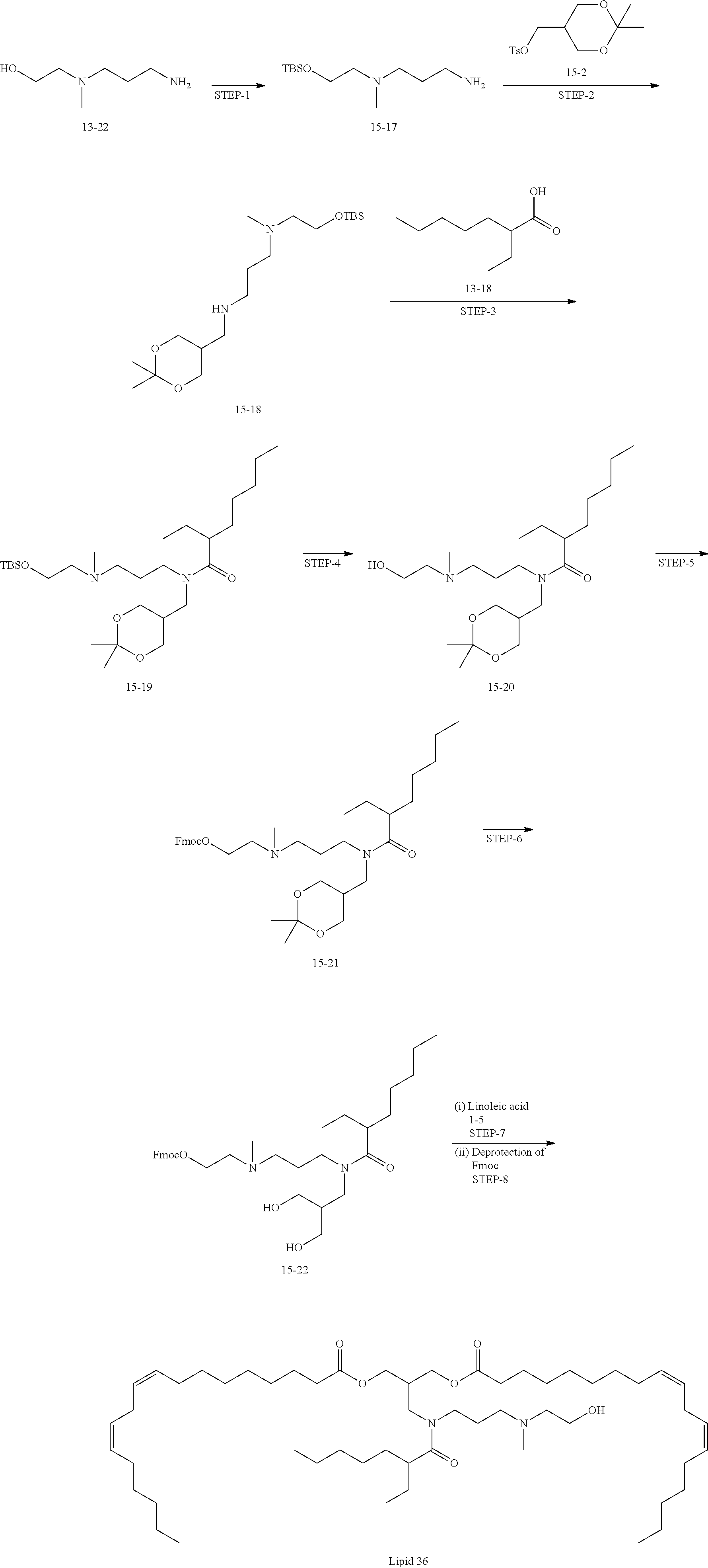

### Synthesis of Lipid 37A

Benzyl protected malonic acid 13-32 (5.1 mmol, 1.0 g, 1 eq.) was esterified with N-hexanol (0.78 g, 1.5 eq, 7.7 mmol) using HATU (2.93 g, 1.5 eq, 7.7 mmol) and DBU (1.56 g, 2.0 eq, 10.3 mmol) in 8 mL DMF for 16 hours at room temperature yielding 0.5 g (35%) of protected intermediate ester 14-9. A second 5 g scale batch using the same reaction conditions and reagent stoichiometry yielded an additional 6 g (84%) of intermediate 14-9. Intermediate 14-18 was produced via acylation of bromoacetyl bromide 13-35 with N-decanol.

Intermediate 14-9 (21.6 mmol, 6 g, 1 eq.) was alkylated with 14-18 (7.2 g, 1.2 eq, 26.0 mmol) using sodium hydride NaH (1.0 g, 1.2 eq, 26.0 mmol) in 40 mL DMF at room temperature overnight to obtain protected intermediate 14-19 (2.5 g, 25%). Intermediate 14-19 (2.5 g, 1.0 eq.) was deprotected using Pd/C (500 mg, 32% w/w) in 30 mL Ethyl Acetate at room temperature overnight yielding 2.02 g (99%) of acid intermediate 14-20.

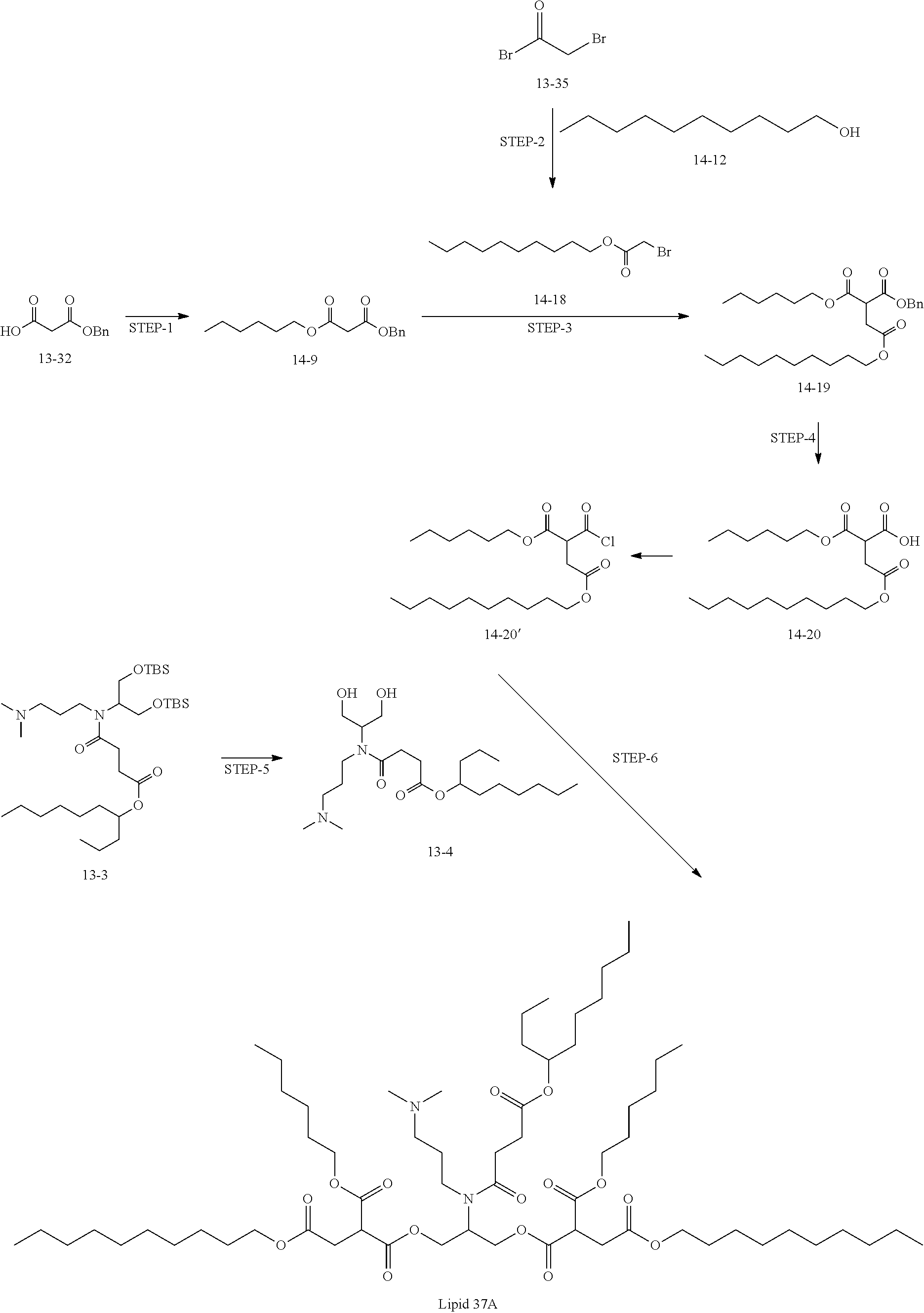

Acid intermediate 14-20 (2.7 mmol, 1.05 g, 1 eq.) was converted to the corresponding acid chloride 14-20′ using oxalyl chloride (0.79 mL, 3.4 eq, 9.2 mmol) and DMF (100 μL, catalytic quantity) in 6.0 mL of Toluene at room temperature for 2 hours and conversion to chloride intermediate was confirmed by TLC.

Intermediate 13-4 (1.57 mmol, 195 mg) was reacted with crude 14-20′ (2.83 g, 6.0 eq, 9.4 mmol) using TEA (2.18 mL, 10.0 eq, 15.7 mmol) in 16.0 mL of Toluene at room temperature overnight. Crude product was purified on ISCO column chromatography on silica column eluting with DCM and 10% MeOH in DCM twice to obtain 205 mg (38%) of >99% pure (HPLC-CAD) Lipid 37A. (see FIG. 4AH-1 and FIG. 4AH-2 for characterization by proton NMR and LC-CAD purity, and Table 5 for Mass Spectrometry data).

### Example 2. Preparation of LNPs by Microfluidic Mixing Using Exemplary Ionizable Lipids

Exemplary LNPs were produced using cationic Lipid 9 and cationic Lipid 15 as synthesized in Example 1.

LNPs encapsulating an mRNA payload were prepared by mixing an aqueous mRNA solution and an ethanolic lipid blend solution (containing ionizable lipid, DSPC, DPG-PEG and Cholesterol at lipid ratios shown in Table 6) using an in-line microfluidic mixing process. The mRNA (eGFP encoding mRNA, TriLink Biotechnologies, California, US) stock solution (1 mg/mL) was diluted in pH 4 acetate buffer (yielding a 133 μg/mL solution of mRNA) in 21.7 mM pH 4 acetate buffer. The lipid components were dissolved in anhydrous ethanol at the relative ratios set forth in TABLE 6 below.

TABLE 6

| | | Ratio of Lipid | Concentration | Theoretical |
|---|---|---|---|---|
| | | to mRNA | in Lipid | LNP Lipid |
| | | (nmol lipid/ | Solution | Composition |
| Lipid | Source | 100 μg mRNA) | (mM) | (mol %) |
| Ionizable Cationic | — | 1,500 | 6 | 49.2 |
| Lipid | | | | |
| Cholesterol | Dishman | 1,200 | 4.8 | 39.4 |
| | Netherlands | | | |
| DSPC | Avanti Polar Lipids, | 300 | 1.2 | 9.8 |
| | Alabama, U.S. | | | |
| DPG-PEG(2000) | NOF America, | 46 | 0.18 | 1.5 |
| | New York, U.S. | | | |
| DiIC18(5)-DS | Invitrogen, | 1.8 | 0.007 | 0.06 |
| | Massachusetts, US | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution (1.5 mL) to lipid solution (0.5 mL) at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM pH 7.4 HEPES buffer with 150 mM NaCl). The LNP suspension (2 mL) was then loaded into the device and centrifuged at 500 RCF until the volume was reduced by half volume (1 mL). The suspension was then diluted with exchange buffer (1 mL, 25 mM pH 7.4 HEPES buffer) to bring the suspension back to the original volume. This process of two-fold concentration and two-fold dilution was repeated five additional times for a total of six discontinuous diafiltration steps. The LNP suspension was then exchanged into MBS (25 mM pH 6.5 MES buffer with 150 mL NaCl) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

### Example 3. Characterization of LNPs

This Example describes the characterization of LNPs produced in Example 2.

Samples of the LNPs produced in Example 2 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both un-encapsulated RNA and accessible RNA at the LNP surface, is measured by diluting the nanoparticles to approximately 1 μg/mL mRNA using HEPES buffered saline, and then adding Quant-iT reagent to the mixture. Total RNA content is measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA is quantified by measuring fluorescence at 485/535 nm, and concentration is determined relative to a contemporaneously run RNA standard curve. The results are set forth in TABLE 7.

TABLE 7

| | | DLS | | Zeta | Zeta | Dve- |
|---|---|---|---|---|---|---|
| | | Z-Avg. | | Potential | Potential | Accessible |
| Formulation | Ionizable | Diameter | DLS | at pH 5.5 | at pH 7.4 | mRNA |
| No. | Lipid | (nm) | PDI | (mV) | (mV) | (%) |
| 1 | Cationic Lipid 9 | 95 | 0.07 | 17 | 0.0 | 5 |
| 2 | Cationic Lipid 15 | 98 | 0.06 | 20 | 0.3 | 5 |

### Example 4. Preparation of Fab Conjugates to Enable T-Cell Targeting

This Example describes the production of an exemplary lipid-immune cell targeting group conjugate.

An anti-CD3 Fab (hSP34 with mouse lambda and human lambda) (see amino acid sequences below) was conjugated to DSPE-PEG (2k)-maleimide via covalent coupling between the maleimide group and a C-terminal cysteine in the heavy chain (HC). Anti-CD3 Fab clone Hu291, anti-CD8 Fab clone TRX2, anti-CD8 Fab clone OKT8, a non-functional mutated OKT8 (mutOKT8), anti-CD4 Fab from Ibalizumab sequence, anti-CD5 Fab clone He3, anti-CD7 Fab clone TH-69, anti-CD2 Fab clone TS2/18.1, anti-CD2 Fab clone 9.6, anti-CD2 Fab clone 9-1 with human kappas were also conjugated using similar methods described herein. The protein (3-4 mg/mL), after buffer exchange into oxygen free, pH 7 phosphate buffer, was reduced in 2 mM TCEP in oxygen free pH 7 phosphate buffer for 1 hour at room temperature. The reduced protein was isolated using a 7 kDa SEC column to remove TCEP and buffer exchanged into fresh oxygen free pH 7 phosphate buffer.

The conjugation reaction was initiated by addition of a 10 mg/mL micellar suspension of DSPE-PEG-maleimide (Avanti Polar Lipids, Alabama, US) and 30 mg/mL DSPE-PEG-OCH₃ (Avanti Polar Lipids, Alabama, U.S.) (1:1 to 1:3 weight ratio is used depending on protein) in oxygen free pH 5.7 citrate buffer (1 mM Citrate). Protein solution is concentrated to 3-4 mg/mL using a 10 kDa Regenerated Cellulose Membrane and subsequently buffer exchanged in oxygen free pH 7 phosphate buffer using a 40 kDa Size Exclusion Column. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG (2k)-anti-hSP34 Fab) is isolated using a 100 kDa Millipore Regenerated Cellulose membrane filtration using pH 7.4 HEPES buffer saline (25 mM HEPES, 150 mM NaCl) buffer and stored at 4° C. prior to use. After quenching, the final micelle composition consists of a mixture of DSPE-PEG-Fab, DSPE-PEG-maleimide (cysteine terminated), and DSPE-PEG-OCH₃. The ratio of the three components is DSPE-PEG-Fab:DSPE-PEG-maleimide (cysteine terminated):DSPE-PEG-OCH₃=1:2.45:3.45-10.35 (by mole)).

The resulting conjugate displayed comparable binding to recombinant Rhesus CD3 epsilon as the unconjugated anti-CD3 Fab by ELISA assay.

Sequences of antibodies in this example are provided in International Patent Application NO. PCT/US2023/068090, which is incorporated by reference in its entirety.

### Example 5. Preparation of LNPs Containing T Cell Targeting Group

This Example describes the incorporation of an immune cell targeting conjugate into a preformed LNP.

LNPs from Example 2 and conjugates (anti-CD3 (hSP34) and anti-CD8 (TRX-2) conjugates) were prepared using methods described in Example 4 were combined as shown in Table 8 in an Eppendorf tube and vortexed for 10 seconds at 2,500 rpm. The Eppendorf tubes were placed in the ThermoMixer at 37° C. at 300 rpm for 4 hours. Resulting targeted LNP suspension was subsequently stored at 4° C. until use or alternatively stored frozen after reconstitution into sucrose medium at final sucrose concentration of 9.6 wt. % by dilution using the appropriate volume of a 50 wt. % sucrose stock solution (in HEPES buffer saline; 25 mM HEPES, 150 mM NaCl)) and stored frozen at −80° C.

TABLE 8

| | Target FAb | | RNA | FAb | | |
|---|---|---|---|---|---|---|
| | density in | FAb | Conc. In | Conjugate | Fab | LNP |
| nmol total lipid/ | targeted LNP g | mg/mg | LNP | Conc. | mg/mL | mL/mL |
| mg RNA | (Fab/mol lipid) | RNA | (mg/mL) | (mg/mL) | LNP | Fab |
| 30,460 | 9 | 0.274 | 0.45 | 1.46 | 0.123 | 11.8 |
| 30,460 | 3 | 0.091 | 0.45 | 1.46 | 0.041 | 35.5 |

### Example 6. Preparation of LNPs by Microfluidic in-Line Mixing and Tangential Flow Filtration Using an Exemplary Ionizable Lipid

This example describes preparation of LNPs using scalable unit operations, namely in-line microfluidic mixing followed by tangential flow filtration (TFF) for ethanol removal and buffer exchange. Separate LNP batches were prepared using either DLin-KC3-DMA or Lipid 15 as the ionizable lipid.

LNPs encapsulating an mRNA payload were prepared by mixing an aqueous mRNA solution and an ethanolic lipid blend solution using an in-line microfluidic mixing process. The mRNA (eGFP or mCherry encoding mRNA, TriLink Biotechnologies, California, US) stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in TABLE 9.1 below.

TABLE 9.1

| | | Ratio of Lipid | Concentration | Theoretical |
|---|---|---|---|---|
| | | to mRNA | in Lipid | LNP Lipid |
| | | (nmol lipid/ | Solution | Composition |
| Lipid | Source | 100 μg mRNA) | (mM) | (mol %) |
| Ionizable Cationic | — | 1,500 | 18.0 | 49.2 |
| Lipid | | | | |
| Cholesterol | Dishman | 1,200 | 14.4 | 39.4 |
| | Netherlands | | | |
| DSPC | Avanti Polar Lipids, | 300 | 3.6 | 9.8 |
| | Alabama, U.S. | | | |
| DPG-PEG(2000) | NOF America, | 46 | 0.55 | 1.5 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution to lipid solution at a total flow rate of 9 mL/min using the NanoAssemblr Ignite. The resulting suspension was held at room temperature for a minimum of 5 minutes before proceeding to ethanol removal and buffer exchange. Ethanol removal and buffer exchange were subsequently performed using constant volume tangential flow filtration (TFF).

Following mixing, ethanol removal and buffer exchange were performed on the resulting LNP suspension using a hollow fiber TFF module (mPES membrane with 300 kDa MWCO, Repligen, US). Briefly, the TFF module was rinsed with DI water and pumped dry before use. The buffer selected for use as the diafiltration buffer depended on the ionizable lipid in the LNP formulation. For DLin-KC3-DMA LNPs, 25 mM pH 7.4 HEPES buffer with 150 mM NaCl (HBS) was used as the diafiltration buffer. For Lipid 15 LNPs, 25 mM pH 6.5 MES buffer with 150 mM NaCl (MBS) was used as the diafiltration buffer. The LNP mixture in ethanol/water solution was added to the reservoir, the TFF module was primed, and diafiltration was initiated by ramping up the peristaltic pump to the target flow rate and adjusting the retentate valve until the target transmembrane pressure (TMP) was reached. A shear rate of 8000 s⁻¹ and a TMP of 3.5 psi were the target operating parameters for the system once diafiltration was initiated. Throughout the diafiltration process, the TMP was kept constant by adjusting the retentate valve. Permeate flux was monitored and was >20 LMH throughout diafiltration. Six diafiltration exchange volumes were performed, with samples set aside at the end of each diafiltration volume to later track the buffer exchange process. Final ethanol content was ≤0.1%, as measured by refractive index measurements on permeate samples, and pH measurements confirmed the buffer exchange into the desired diafiltration buffer. Upon the completion of six diafiltration volumes, a concentration of the resulting LNP suspension was subsequently performed.

The concentration of the LNP suspension to a target total mRNA concentration of ~0.8 mg/mL was performed using the same TFF module that was used during the buffer exchange process. TMP and flow rate during the buffer exchange process were maintained, and the suspension was allowed to concentrate by stopping the addition of diafiltration buffer to the retentate reservoir. The resulting LNP suspension was collected and filtered with a 0.2 μm syringe filter. The suspension was sampled for analytical purposes and then stored at 4° C. until further use.

Using the LNP characterization process in Example 3, LNP batch was characterized to determine the average hydrodynamic diameter and mRNA content (total and dye-accessible); set forth in Table 9 below. As seen in Table 9.2, the microfluidic mixing process with ethanol removal and buffer exchange by TFF results in sub-100 nm particles exhibiting narrow polydispersity and good mRNA encapsulation (<20% dye accessible RNA).

TABLE 9.2

| | | | | | Dye- | |
|---|---|---|---|---|---|---|
| | | DLS | | Total | Accessible | Dye- |
| | | Z-Avg. | | mRNA | mRNA | Accessible |
| Sample ID/ | | Diameter | DLS | Content | Content | mRNA |
| Lot Number | Description | (nm) | PDI | (μg/mL) | (μg/mL) | (%) |
| EXP22001562- | DLin-KC3-DMA | 85 | 0.07 | 846 | 115 | 14 |
| NF40 | LNPs after 6 | | | | | |
| | DVs in HBS | | | | | |
| EXP22007940- | Lipid 15 | 69 | 0.14 | 990 | 161 | 16 |
| NU400 | LNPs after 6 | | | | | |
| | DVs in MBS | | | | | |

### Example 7. Method for Determination of the LNP Apparent pKa Using the Toluidinyl-Naphthalene Sulfonate (TNS) Fluorescent Probe

This example describes the fluorescent dye-based method used for measurement of the apparent pKa of the lipid nanoparticles. Apparent pKa determines the nanoparticle surface charge under physiological pH conditions, typically a pKa value in the endosomal pH range (6-7.4) results in LNPs that are neutral or slightly charged at plasma or the extracellular space (pH 7.4) and become strongly positive under acidic endosomal environments. This positive surface charge drives fusion of the LNP surface with negatively charged endosomal membranes resulting in destabilization and rupture of the endosomal compartment and LNP escape into the cytosolic compartment, a critical step in cytosolic delivery of mRNA and protein expression via engagement of the cells ribosomal machinery.

The apparent pKa of LNPs is determined by 6-(p-Toluidino)-2-naphthalenesulfonic acid (TNS) fluorescence measurement in aqueous buffers covering a range of pH values (pH 4-pH 10). TNS dye is non-fluorescent when free in solution, but fluoresces strongly when associated with a positively charged lipid nanoparticle. At pH values below the pKa of the nanoparticle, positive LNP surface charge results in dye recruitment at the particle interface resulting in TNS fluorescence. At pH values above the LNP pKa, the LNP surface charge is neutralized and TNS dye dissociates away from the particle interface resulting in loss of fluorescence signal. The apparent pKa of the LNP is reported as the pH at which the fluorescence is at 50% of its maximum, as determined using a four-point logistic curve fit.

### Example 8. General Formulation and Physiochemical Characterization Methodology for mRNA Encapsulating LNPs Based on Lipids 1-8, 9-15 and 31-34

Lipid nanoparticles (LNPs) bearing a nucleic acid (reporter RNA or CAR RNA) were formulated by a microfluidic mixing process using lipid and solvent compositions described in Example 2 and 6 above and buffer exchanged into pH 7.4 HEPES buffer saline (resulting in ethanol removal and pH adjustment) using either centrifugal ultrafiltration membrane filter devices or a tangential flow filtration (TFF) process; and characterized by Dynamic Light Scattering (DLS) for hydrodynamic size (diameter, nm), polydispersity (PDI) and charge at pH 5.5 and pH 7.4 (Zeta Potential, mV). The mRNA encapsulation efficiency (percent dye accessible RNA) and total mRNA content (ug/mL RNA in LNP suspension) were determined using methods described in Example 3. The formulated LNPs were subsequently buffer exchanged into pH 6.5 MES buffer saline and the size distribution was re-characterized by DLS prior to mixing with the desired quantity of targeting antibody conjugate (see Table 8, Example 5) and incubated at 37° C. for 4 hours to facilitate antibody insertion (using process described in Example 5) resulting in final antibody targeted LNPs. The obtained targeted LNPs were sterile filtered and characterized by DLS (size (nm) and PDI) using methods described in Example 3.

### Example 9. Physiochemical Properties (Pre- and Post-Insertion with αCD3 Fab Conjugate Hsp34) of Lipids 1-8 GFP RNA Lipid Nanoparticles (LNPs)

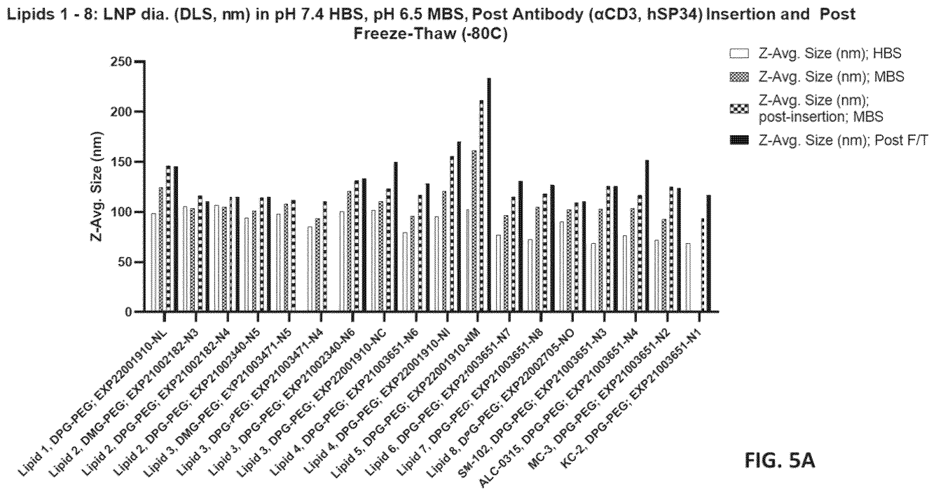

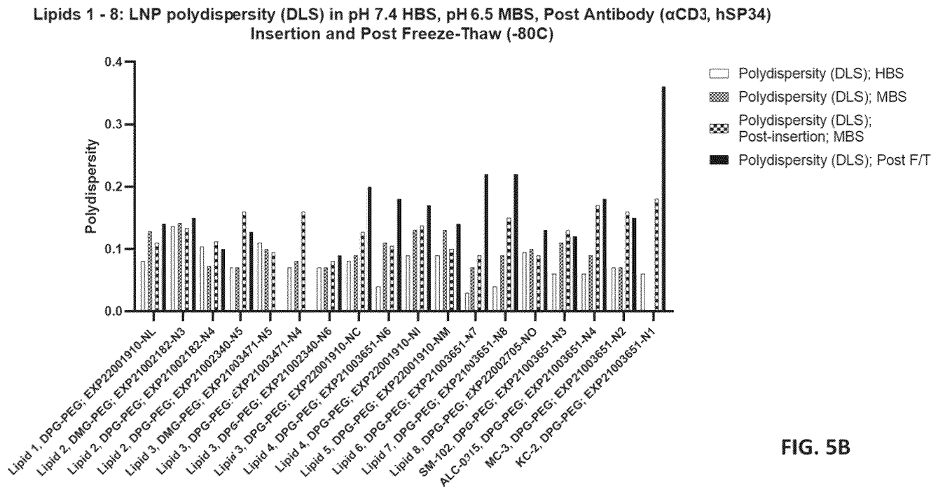

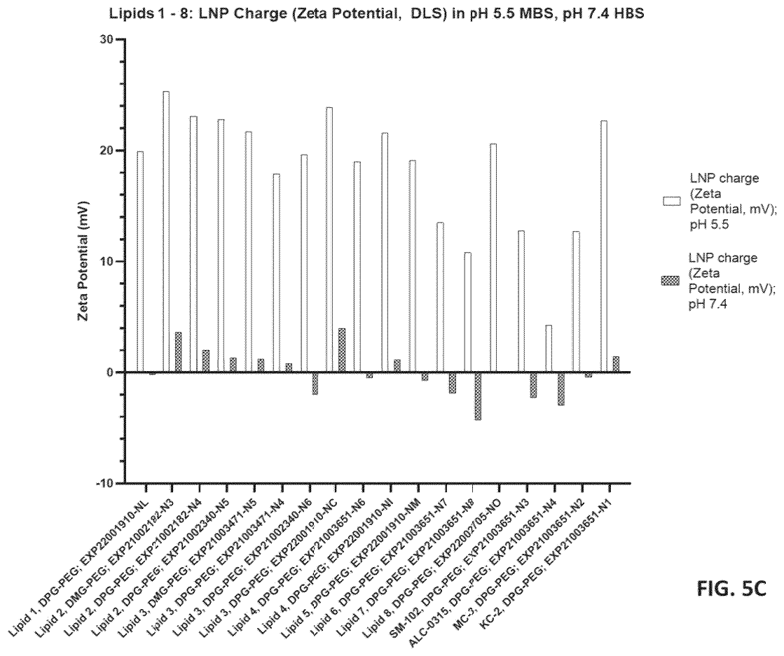

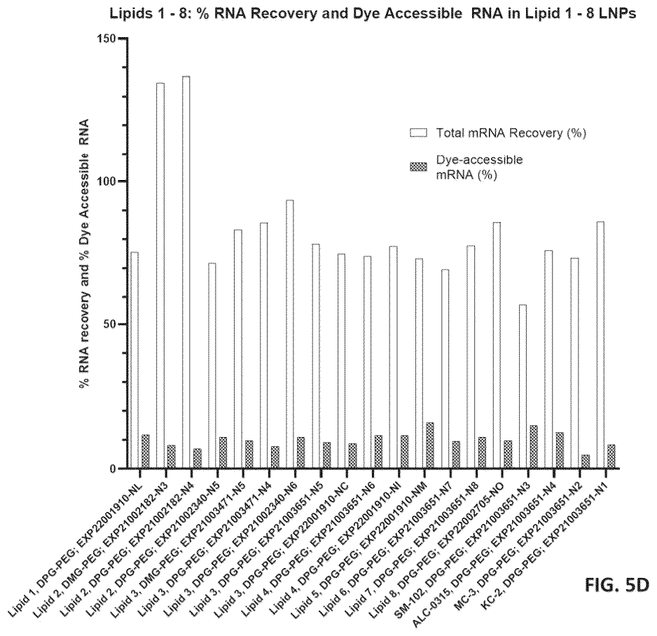

Lipids 1, 2, 3, 4, 5, 6, 7 and 8 LNPs encapsulating either GFP-mRNA (TriLink Biotechnologies Inc.) or CAR-RNA (custom made by TriLink Biotechnologies Inc.) were prepared and characterized using methods described in Examples 5 to 8. Measured LNP size, PDI, charge and RNA content values for Lipid 1-8 LNP and LNPs made using comparator ionizable lipids (including MC3, KC2, SM-102, and ALC-0315) are summarized in Table 10 to Table 12. As seen in FIG. 5A, the initial mixing step and subsequent buffer exchange into HBS resulted in Lipid 1-8 LNP sizes in the 80-120 nm range. All lipids resulted in high encapsulation efficiency (<15% Dye accessible RNA) and >70% RNA recovery. The subsequent buffer exchange into pH 6.5 MES buffer and antibody insertion process (37° C., 4 hours) was well tolerated by lipids 2, 3, 6, 7 and 8 LNPs resulting in targeted LNP diameters below 140 nm and PDI<0.2. Lipids 1 and 4 LNPs exhibited relatively larger size shifts resulting in final targeted LNPs in the 140-160 nm diameter range, however particles size distributions remained narrow and monomodal for all lipids tested. Lipid 5 LNPs exhibited the greatest size shift with in final targeted LNPs exhibiting>160 nm diameter. Lipids 1, 2, 6, 7 and 8 exhibited no significant size and polydispersity changes after one Freeze-Thaw cycle, however, moderate changes were observed with LNPs based on lipids 3, 4, and 5. In summary, all lipids tested resulted in viable mRNA encapsulation and freeze-thaw stability as well as <200 nm final targeted LNP diameters. The ability of lipids 1-8 LNPs for inducing in vitro protein transfection in primary human T-cells mediated by αCD3 or αCD8 T-cell receptor targeting was evaluated as described in Examples 16 and 17.

TABLE 10
Lipids 1-8 and comparator Lipids LNP Size, Polydispersity (DLS) data in pH
7.4 HBS, pH 6.5 MBS and post αCD3 antibody Fab (hSP34) conjugate insertion

| | | | Z-Avg. | | | | Polydispersity | |
|---|---|---|---|---|---|---|---|---|
| | Z-Avg. | Z-Avg. | Size (nm); | Z-Avg. | Polydispersity | Polydispersity | (DLS); | Polydispersity |
| Ionizable Lipid, | Size (nm); | Size (nm); | post-insertion; | Size (nm): | (DLS); | (DLS); | Post-insertion; | (DLS); |
| LNP no. | HBS | MBS | MBS | Post F/T | HBS | MBS | MBS | Post F/T |
| Lipid 1, | 98.78 | 124.3 | 146.2 | 145.5 | 0.08 | 0.128 | 0.11 | 0.14 |
| DPG-PEG; | | | | | | | | |
| EXP22001910- | | | | | | | | |
| NL | | | | | | | | |
| Lipid 2, | 105.4 | 103.95 | 116.5 | 110.5 | 0.136 | 0.142 | 0.133 | 0.15 |
| DMG-PEG; | | | | | | | | |
| EXP21002182- | | | | | | | | |
| N3 | | | | | | | | |
| Lipid 2, | 107 | 105 | 115.2 | 115.1 | 0.104 | 0.072 | 0.112 | 0.1 |
| DPG-PBG; | | | | | | | | |
| EXP21002182- | | | | | | | | |
| N4 | | | | | | | | |
| Lipid 2, | 94 | 101.3 | 114.7 | 114.8 | 0.07 | 0.07 | 0.16 | 0.127 |
| DPG-PEG; | | | | | | | | |
| EXP21002340- | | | | | | | | |
| N5 | | | | | | | | |
| Lipid 3, | 98.1 | 107.8 | 111.8 | | 0.11 | 0.1 | 0.095 | |
| DMG-PEG; | | | | | | | | |
| EXP21003471- | | | | | | | | |
| N5 | | | | | | | | |
| Lipid 3, | 85.3 | 93.7 | 110.4 | | 0.07 | 0.08 | 0.16 | |
| DPG-PEG; | | | | | | | | |
| EXP21003471- | | | | | | | | |
| N4 | | | | | | | | |
| Lipid 3, | 100.7 | 120.5 | 131.3 | 133.3 | 0.07 | 0.07 | 0.08 | 0.09 |
| DPG-PEG; | | | | | | | | |
| EXP21002340- | | | | | | | | |
| N6 | | | | | | | | |
| Lipid 3, | 101.5 | 110.8 | 123.6 | 150.2 | 0.08 | 0.09 | 0.127 | 0.2 |
| DPG-PEG; | | | | | | | | |
| EXP22001910- | | | | | | | | |
| NC | | | | | | | | |
| Lipid 4, | 79.5 | 96.3 | 116.8 | 128.5 | 0.04 | 0.11 | 0.105 | 0.18 |
| DPG-PEG; | | | | | | | | |
| EXP21003651- | | | | | | | | |
| N6 | | | | | | | | |
| Lipid 4, | 95.5 | 120.6 | 155.4 | 170.3 | 0.09 | 0.13 | 0.137 | 0.17 |
| DPG-PEG; | | | | | | | | |
| EXP22001910- | | | | | | | | |
| NI | | | | | | | | |

TABLE 11
Lipids 1-8 LNP charge, Zeta Potential, ZP
(DLS, mV) in pH 5.5 MES and pH 7.4 HBS

| | Charge | Charge |
|---|---|---|
| | (ZP, mV); | (ZP, mV); |
| Ionizable Lipid, LNP no. | pH 5.5 | pH 7.4 |
| Lipid 1, DPG-PEG; EXP22001910-NL | 19.9 | −0.212 |
| Lipid 2, DMG-PEG; EXP21002182-N3 | 25.3 | 3.64 |
| Lipid 2, DPG-PEG; EXP21002182-N4 | 23.1 | 2.03 |
| Lipid 2, DPG-PEG; EXP21002340-N5 | 22.8 | 1.29 |
| Lipid 3, DMG-PEG; EXP21003471-N5 | 21.7 | 1.18 |
| Lipid 3, DPG-PEG; EXP21003471-N4 | 17.9 | 0.776 |
| Lipid 3, DPG-PEG; EXP21002340-N6 | 19.6 | −2.01 |
| Lipid 3, DPG-PEG; EXP22001910-NC | 23.9 | 3.99 |
| Lipid 4, DPG-PEG; EXP21003651-N6 | 19 | −0.508 |
| Lipid 4, DPG-PEG; EXP22001910-NI | 21.6 | 1.16 |
| Lipid 5, DPG-PEG; EXP22001910-NM | 19.1 | −0.742 |
| Lipid 6, DPG-PEG; EXP21003651-N7 | 13.5 | −1.9 |
| Lipid 7, DPG-PEG; EXP21003651-N8 | 10.8 | −4.31 |
| Lipid 8, DPG-PEG; EXP22002705-NO | 20.6 | 0.051 |
| SM-102, DPG-PEG; EXP21003651-N3 | 12.8 | −2.25 |
| ALC-0315, DPG-PEG; EXP21003651-N4 | 4.25 | −2.95 |
| MC-3, DPG-PEG; EXP21003651-N2 | 12.7 | −0.43 |
| KC-2, DPG-PEG; EXP21003651-N1 | 22.7 | 1.41 |

TABLE 12
Lipids 1-8 and comparator Lipids LNP Dye
Accessible RNA and total RNA content

| | Nominal | Measured | Dye- | Total | Dye- |
|---|---|---|---|---|---|
| | mRNA | Total | accessible | mRNA | accessible |
| Ionizable Lipid, | Conc. | mRNA | mRNA | Recovery | mRNA |
| LNP no. | (μg/mL) | (μg/mL) | (μg/mL) | (%) | (%) |
| Lipid 1, DPG-PEG; | 100 | 75.5 | 8.9 | 75.5 | 11.8 |
| EXP22001910-NL | | | | | |
| Lipid 2, DMG-PEG; | 100 | 134.7 | 11.1 | 134.7 | 8.2 |
| EXP21002182-N3 | | | | | |
| Lipid 2, DPG-PEG; | 100 | 136.9 | 9.4 | 136.9 | 6.9 |
| EXP21002182-N4 | | | | | |
| Lipid 2, DPG-PEG; | 100 | 71.8 | 7.8 | 71.8 | 10.9 |
| EXP21002340-N5 | | | | | |
| Lipid 3, DMG-PEG; | 100 | 83.2 | 8.1 | 83.2 | 9.8 |
| EXP21003471-N5 | | | | | |
| Lipid 3, DPG-PEG; | 100 | 85.7 | 6.6 | 85.7 | 7.7 |
| EXP21003471-N4 | | | | | |
| Lipid 3, DPG-PEG; | 100 | 93.5 | 10.3 | 93.5 | 11.0 |
| EXP21002340-N6 | | | | | |
| Lipid 3, DPG-PEG; | 100 | 74.9 | 6.5 | 74.9 | 8.7 |
| EXP22001910-NC | | | | | |
| Lipid 4, DPG-PEG; | 50 | 37.1 | 4.3 | 74.2 | 11.5 |
| EXP21003651-N6 | | | | | |
| Lipid 4, DPG-PEG; | 100 | 77.6 | 9 | 77.6 | 11.6 |
| EXP22001910-NI | | | | | |
| Lipid 5, DPG-PEG; | 100 | 73.4 | 11.8 | 73.4 | 16.1 |
| EXP22001910-NM | | | | | |
| Lipid 6, DPG-PEG; | 50 | 34.7 | 3.3 | 69.4 | 9.5 |
| EXP21003651-N7 | | | | | |
| Lipid 7, DPG-PEG; | 50 | 38.9 | 4.3 | 77.8 | 11.1 |
| EXP21003651-N8 | | | | | |
| Lipid 8, DPG-PEG; | 100 | 85.9 | 8.4 | 85.9 | 9.8 |
| EXP22002705-NO | | | | | |
| SM-102, DPG-PEG; | 50 | 28.7 | 4.3 | 57.4 | 15.0 |
| EXP21003651-N3 | | | | | |
| ALC-0315, DPG-PEG; | 50 | 38.1 | 4.8 | 76.2 | 12.6 |
| EXP21003651-N4 | | | | | |
| MC-3, DPG-PEG; | 50 | 36.8 | 1.8 | 73.6 | 4.9 |
| EXP21003651-N2 | | | | | |
| KC-2, DPG-PEG; | 50 | 43 | 3.6 | 86 | 8.4 |
| EXP21003651-N1 | | | | | |

### Example 10. Physiochemical Properties (Pre- and Post-Insertion with αCD3 Fab Conjugate HSP34) of Lipids 9, 10, 11 and 15 GFP RNA Lipid Nanoparticles (LNPs)

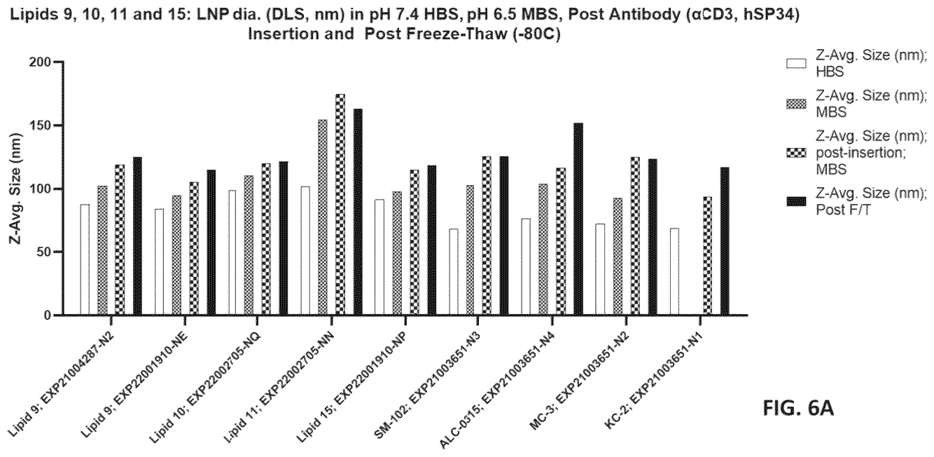

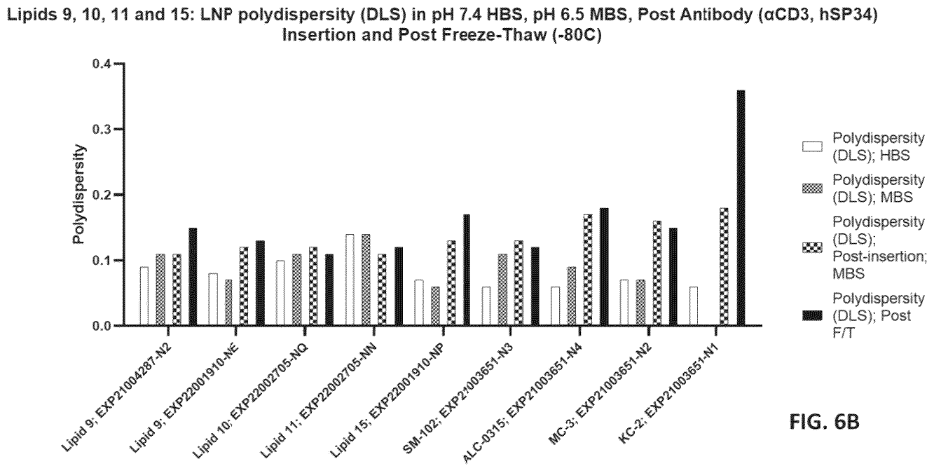

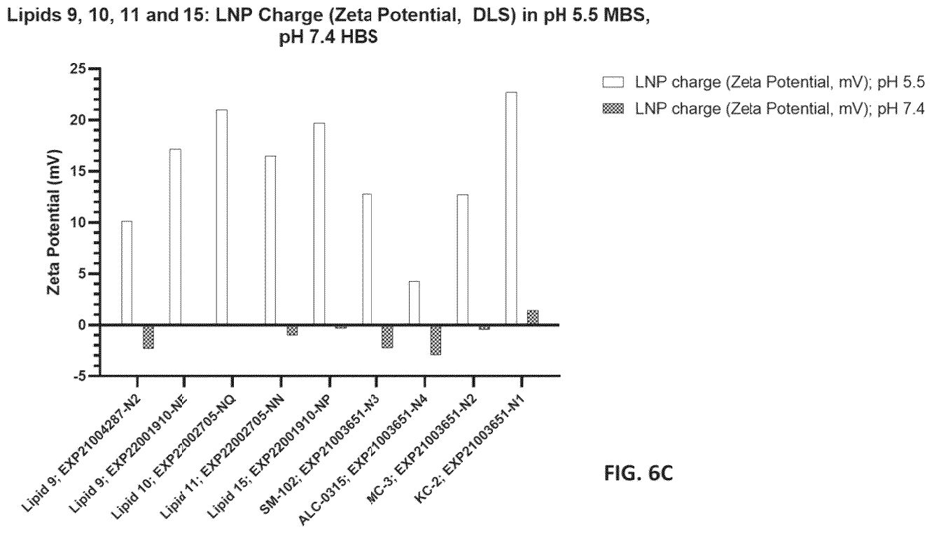

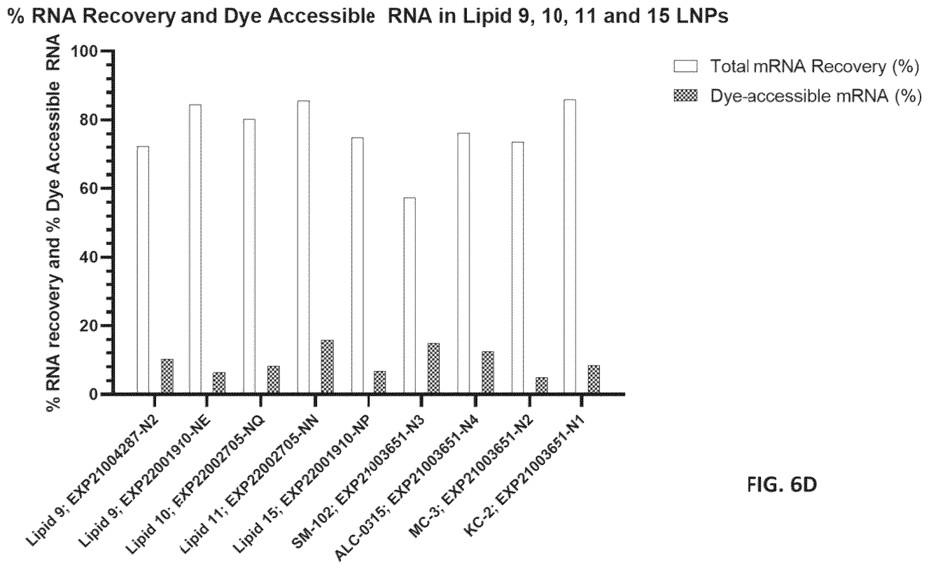

Lipids 9, 10, 11, and 15 LNPs encapsulating either GFP-mRNA (TriLink Biotechnologies Inc.) or CAR-RNA (custom made by TriLink Biotechnologies Inc.) were prepared and characterized using methods described in Examples 5 to 8. Measured LNP size, PDI, charge and RNA content values of resulting and of LNPs made using comparator ionizable lipids (including MC3, KC2, SM-102, and ALC-0315) are summarized in Table 13 to Table 15. As seen in FIG. 6A, the initial mixing step and subsequent buffer exchange into HBS resulted LNP sizes≤100 nm with Lipids 9, 10, 11 and 15. All lipids resulted in high encapsulation efficiency (<15% Dye accessible RNA) and >70% RNA recovery. The subsequent buffer exchange into pH 6.5 MES buffer and antibody insertion process (37 C, 4 hours) was well tolerated by lipids 9, 10, and 15 LNPs resulting in final targeted LNP diameters below 140 nm and PDI<0.2. Lipid 11 LNPs exhibited the greatest size shift upon buffer exchange into pH 6.5 MES buffer, and a moderately larger size shift upon antibody insertion relative to the other lipids tested. All four tested lipid LNPs were stable to one freeze-thaw cycle with no significant shifts in LNP diameter or polydispersity observed. In summary, all four lipids tested resulted in viable mRNA encapsulation and freeze-thaw stability as well as <200 nm final targeted LNP diameters. The ability of lipids 9, 10, 11 and 15 LNPs for inducing in vitro protein transfection in primary human T-cells mediated by αCD3 or αCD8 T-cell receptor targeting was evaluated as described in Examples 16 and 17.

TABLE 13
Lipids 9, 10, 11, 15 LNPs and comparator Lipids LNP Size, Polydispersity (DLS) data
in pH 7.4 HBS, pH 6.5 MBS and post αCD3 antibody Fab (hSP34) conjugate insertion

| | | | Z-Avg. | | | | | |
|---|---|---|---|---|---|---|---|---|
| | Z-Avg. | Z-Avg. | Size (nm); | Z-Avg. | | | PDI (DLS); | |
| Ionizable Lipid, | Size (nm); | Size (nm); | post-insertion; | Size (nm); | PDI (DLS); | PDI (DLS); | Post-insertion; | PDI (DLS); |
| LNP no. | HBS | MBS | MBS | Post F/T | HBS | MBS | MBS | Post F/T |
| Lipid 9; | 87.6 | 102.4 | 118.9 | 125.3 | 0.09 | 0.11 | 0.11 | 0.15 |
| EXP21004287- | | | | | | | | |
| N2 | | | | | | | | |
| Lipid 9; | 83.9 | 94.8 | 105.1 | 115.1 | 0.08 | 0.07 | 0.12 | 0.13 |
| EXP22001910- | | | | | | | | |
| NE | | | | | | | | |
| Lipid 10; | 98.6 | 110.6 | 119.9 | 121.5 | 0.1 | 0.11 | 0.12 | 0.11 |
| EXP22002705- | | | | | | | | |
| NO | | | | | | | | |
| Lipid 11; | 101.9 | 154.6 | 174.5 | 162.9 | 0.14 | 0.14 | 0.11 | 0.12 |
| EXP22002705- | | | | | | | | |
| NN | | | | | | | | |
| Lipid 15; | 91.55 | 97.65 | 114.8 | 118.3 | 0.07 | 0.06 | 0.13 | 0.17 |
| EXP22001910- | | | | | | | | |
| NP | | | | | | | | |
| SM-102; | 68.4 | 102.9 | 125.7 | 125.6 | 0.06 | 0.11 | 0.13 | 0.12 |
| EXP21003651- | | | | | | | | |
| N3 | | | | | | | | |
| ALC-0315; | 76.5 | 103.9 | 116.7 | 152.2 | 0.06 | 0.09 | 0.17 | 0.18 |
| EXP21003651- | | | | | | | | |
| N4 | | | | | | | | |
| DLin-MC3-DMA; | 72.2 | 92.6 | 124.8 | 123.65 | 0.07 | 0.07 | 0.16 | 0.15 |
| EXP21003651- | | | | | | | | |
| N2 | | | | | | | | |
| DLin-KC2-DMA; | 68.8 | | 93.5 | 116.8 | 0.06 | | 0.18 | 0.36 |
| EXP21003651- | | | | | | | | |
| NI | | | | | | | | |

TABLE 14
Lipids 9, 10, 11, 15 Lipid and comparator Lipid LNP charge,
Zeta Potential, ZP (DLS, mV) in pH 5.5 MES and pH 7.4 HBS

| | LNP charge | LNP charge |
|---|---|---|
| | (ZP, mV); | (ZP, mV); |
| Ionizable Lipid, LNP no. | pH 5.5 | pH 7.4 |
| Lipid 9; EXP21004287-N2 | 10.1 | −2.34 |
| Lipid 9; EXP22001910-NE | 17.2 | 0.0437 |
| Lipid 10; EXP22002705-NQ | 21 | −0.137 |
| Lipid 11; EXP22002705-NN | 16.5 | −1.01 |
| Lipid 15; EXP22001910-NP | 19.7 | −0.322 |
| SM-102; EXP21003651-N3 | 12.8 | −2.25 |
| ALC-0315; EXP21003651-N4 | 4.25 | −2.95 |
| DLin-MC3-DMA; EXP21003651-N2 | 12.7 | −0.43 |
| DLin-KC2-DMA; EXP21003651-N1 | 22.7 | 1.41 |

TABLE 15
Lipids 9, 10, 11, 15 LNP and comparator Lipids
LNP Dye Accessible RNA and total RNA content

| | Nominal | Measured | Dye- | Total | Dye- |
|---|---|---|---|---|---|
| | mRNA | Total | accessible | mRNA | accessible |
| | Conc. | mRNA | mRNA | Recovery | mRNA |
| Ionizable Lipid, LNP no. | (μg/mL) | (μg/mL) | (μg/mL) | (%) | (%) |
| Lipid 9; EXP21004287-N2 | 100 | 72.3 | 7.5 | 72.3 | 10.4 |
| Lipid 9; EXP22001910-NE | 100 | 84.4 | 5.4 | 84.4 | 6.4 |
| Lipid 10; EXP22002705-NQ | 100 | 80.2 | 6.6 | 80.2 | 8.2 |
| Lipid 11; EXP22002705-NN | 100 | 85.6 | 13.6 | 85.6 | 15.9 |
| Lipid 15; EXP22001910-NP | 100 | 74.9 | 5.1 | 74.9 | 6.8 |

### Example 11. Physiochemical Properties (Pre- and Post-Insertion with αCD3 Fab Conjugate HSP34) of Lipids 31-34 GFP RNA Lipid Nanoparticles (LNPs)

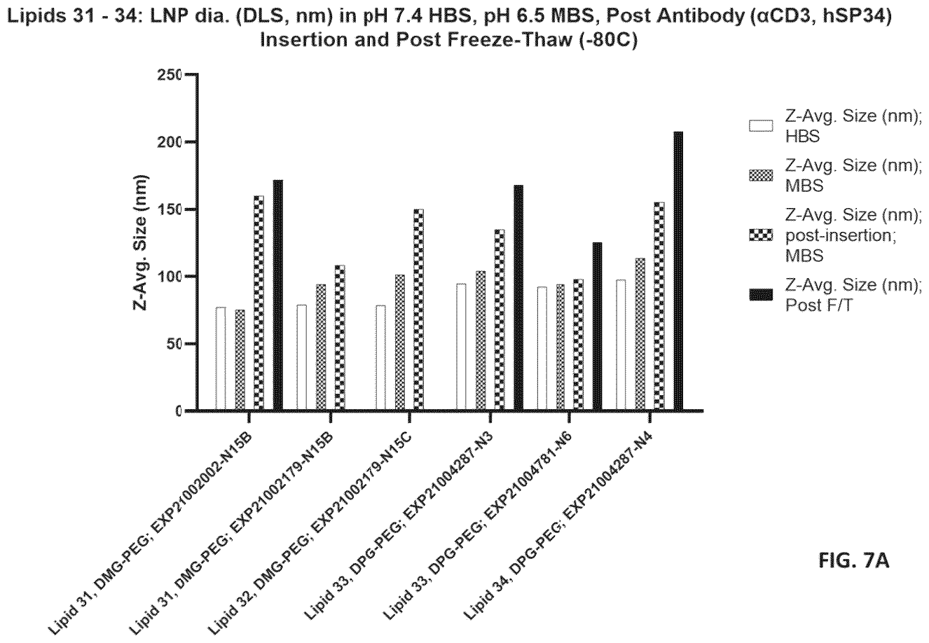

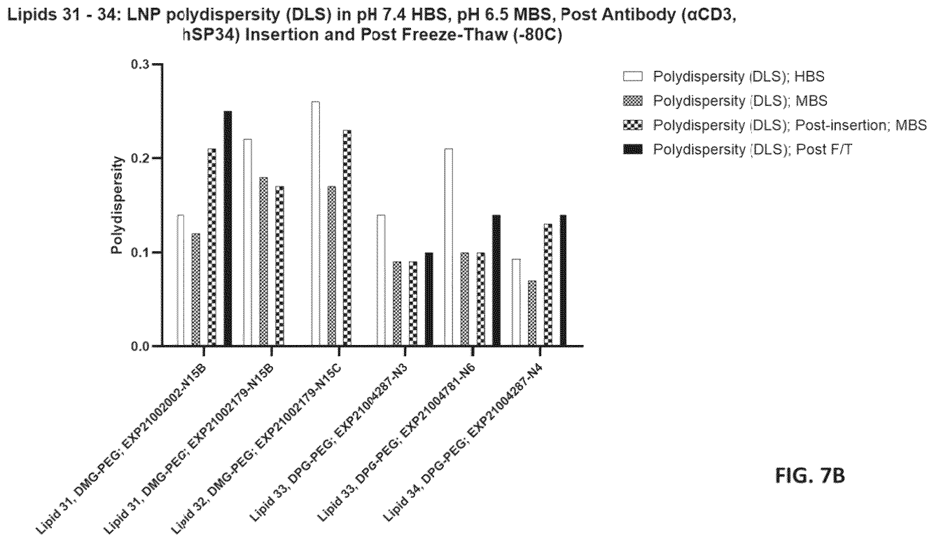

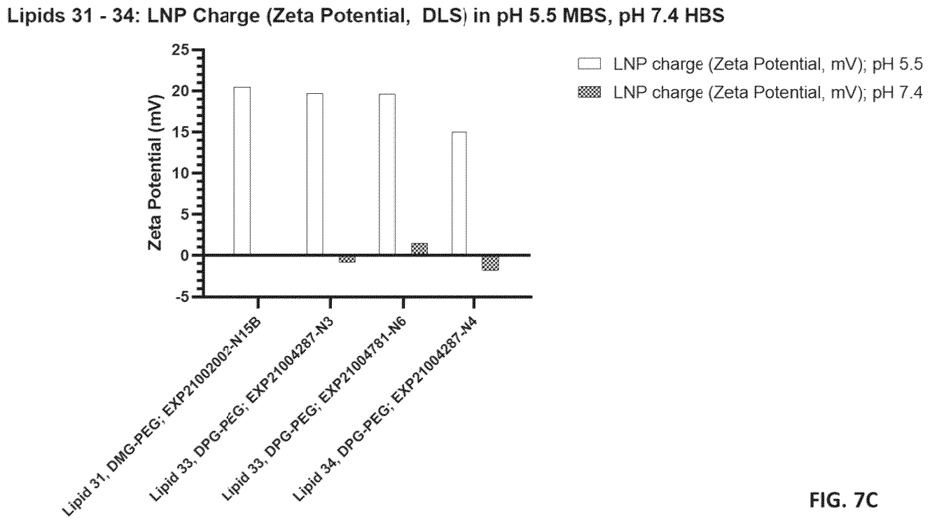

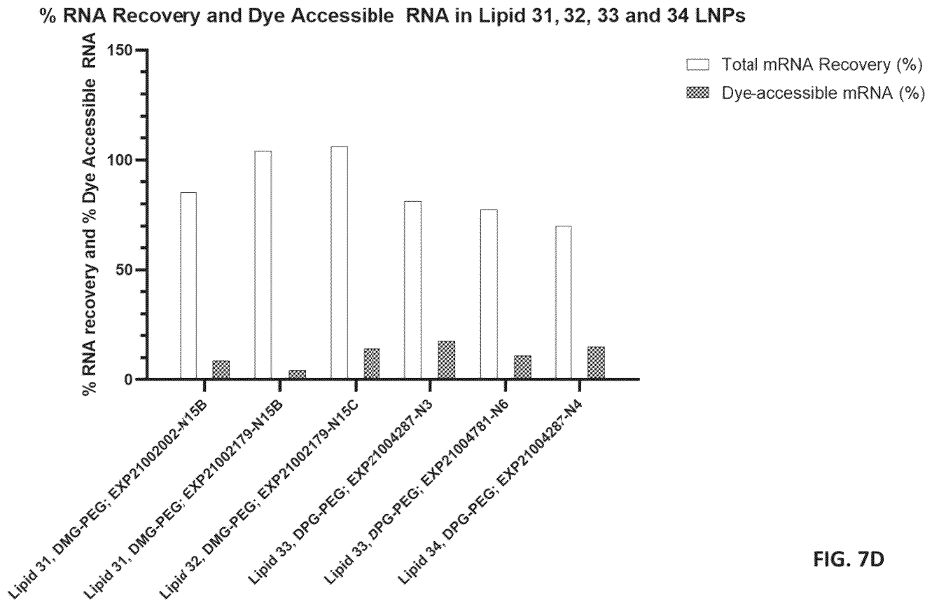

Lipids 31, 32, 33, and 34 LNPs encapsulating either GFP-mRNA (TriLink Biotechnologies Inc.) or CAR-RNA (custom made by TriLink Biotechnologies Inc.) were prepared and characterized using methods described in Examples 5 to 8. Measured LNP size, PDI, charge and RNA content values of Lipids 31, 32, 33, and 34 LNPs are summarized in Table 16 to Table 18. As seen in FIG. 7A, the initial mixing step and subsequent buffer exchange into HBS resulted in LNP sizes≤110 nm with Lipids 31, 32, 33, and 34. All LNPs exhibited moderate to high encapsulation efficiency (<20% Dye accessible RNA) and >70% RNA recovery. The subsequent buffer exchange into pH 6.5 MES buffer and antibody insertion process (37 C, 4 hours) resulted in final targeted LNP diameters in the 120 nm to 160 nm range and PDI in the 0.1-0.25 range indicating relatively poorer particle size control in lipids 31 through 34 LNPs. Lipid 31, 32, and 34 LNPs showed moderate size increases after one freeze-thaw cycle, however, a notably larger size shift was observed with lipid 34 LNPs. In summary, all four lipids tested resulted in viable mRNA encapsulation and freeze-thaw stability as well as <200 nm final targeted LNP diameters. The ability of lipids 31, 32, 33, 34 LNPs for inducing in vitro protein transfection in primary human T-cells mediated by αCD3 or αCD8 T-cell receptor targeting was evaluated as described in Examples 16 and 17.

TABLE 16
Lipids 31, 32, 33, 34 LNPs and comparator Lipids LNP Size, Polydispersity (DLS) data
in pH 7.4 HBS, pH 6.5 MBS and post αCD3 antibody Fab (hSP34) conjugate insertion

| | | | Z-Avg. | | | | Polydispersity | |
|---|---|---|---|---|---|---|---|---|
| | Z-Avg. | Z-Avg. | Size (nm); | Z-Avg. | Polydispersity | Polydispersity | (DLS); | Polydispersity |
| Ionizable lipid, | Size (nm); | Size (nm); | post-insertion; | Size (nm); | (DLS); | (DLS); | Post-insertion; | (DLS); |
| LNP no. | HBS | MBS | MBS | Post F/T | HBS | MBS | MBS | Post F/T |
| Lipid 31, | 77.1 | 75.2 | 159.8 | 171.7 | 0.14 | 0.12 | 0.21 | 0.25 |
| DMG-PEG; | | | | | | | | |
| EXP21002002- | | | | | | | | |
| N15B | | | | | | | | |
| Lipid 31, | 79.23 | 94.3 | 108.3 | ** | 0.22 | 0.18 | 0.17 | |
| DMG-PEG; | | | | | | | | |
| EXP21002179- | | | | | | | | |
| N15B | | | | | | | | |
| Lipid 32, | 78.3 | 101.4 | 149.9 | ** | 0.26 | 0.17 | 0.23 | |
| DMG-PEG; | | | | | | | | |
| EXP21002179- | | | | | | | | |
| N15C | | | | | | | | |
| Lipid 33, | 94.74 | 103.9 | 134.8 | 167.8 | 0.14 | 0.09 | 0.09 | 0.1 |
| DPG-PEG; | | | | | | | | |
| EXP21004287- | | | | | | | | |
| N3 | | | | | | | | |
| Lipid 33, | 92.2 | 94.1 | 98.085 | 125.3 | 0.21 | 0.1 | 0.10 | 0.14 |
| DPG-PEG; | | | | | | | | |
| EXP21004781- | | | | | | | | |
| N6 | | | | | | | | |
| Lipid 34, | 97.31 | 113.3 | 155.1 | 207.5 | 0.093 | 0.07 | 0.13 | 0.14 |
| DPG-PEG; | | | | | | | | |
| EXP21004287- | | | | | | | | |
| N4 | | | | | | | | |

** Not measured.

TABLE 17
Lipids 31, 32, 33, 34 Lipid and comparator Lipid LNP charge,
Zeta Potential, ZP (DLS, mV) in pH 5.5 MES and pH 7.4 HBS

| | Charge | Charge |
|---|---|---|
| | (ZP, mV); | (ZP, mV); |
| Ionizable lipid, LNP no. | pH 5.5 | pH 7.4 |
| Lipid 31, DMG-PEG; EXP21002002-N15B | 20.5 | −0.138 |
| Lipid 31, DMG-PEG; EXP21002179-N15B | | |
| Lipid 32, DMG-PEG; EXP21002179-N15C | | |
| Lipid 33, DPG-PEG; EXP21004287-N3 | 19.7 | −0.867 |
| Lipid 33, DPG-PEG; EXP21004781-N6 | 19.6 | 1.5 |
| Lipid 34, DPG-PEG; EXP21004287-N4 | 15 | −1.79 |

TABLE 18
Lipids 31, 32, 33, 34 LNP and comparator Lipids
LNP Dye Accessible RNA and total RNA content

| | Nominal | Measured | Dye- | Total | Dye- |
|---|---|---|---|---|---|
| | mRNA | Total | accessible | mRNA | accessible |
| | Conc. | mRNA | mRNA | Recovery | mRNA |
| Ionizable lipid, LNP no. | (μg/mL) | (μg/mL) | (μg/mL) | (%) | (%) |
| Lipid 31, DMG-PEG; | 50 | 42.7 | 3.7 | 85.4 | 8.7 |
| EXP21002002-N15B | | | | | |
| Lipid 31, DMG-PEG; | 50 | 52.13 | 2.2 | 104.26 | 4.2 |
| EXP21002179-N15B | | | | | |
| Lipid 32, DMG-PEG; | 50 | 53.02 | 7.5 | 106.04 | 14.1 |
| EXP21002179-N15C | | | | | |
| Lipid 33, DPG-PEG; | 100 | 81.2 | 14.3 | 81.2 | 17.6 |
| EXP21004287-N3 | | | | | |
| Lipid 33, DPG-PEG; | 150 | 116.4 | 12.8 | 77.6 | 110 |
| EXP21004781-N6 | | | | | |
| Lipid 34, DPG-PEG; | 100 | 70 | 10.5 | 70 | 15 |
| EXP21004287-N4 | | | | | |

### Example 12. Physiochemical Properties (Pre- and Post-Insertion with αCD8 Fab Conjugates TRX-2 and 15C01) of Lipids 1, 3, 4, 5, 9 and 15 GFP RNA Containing Lipid Nanoparticles (LNPs)

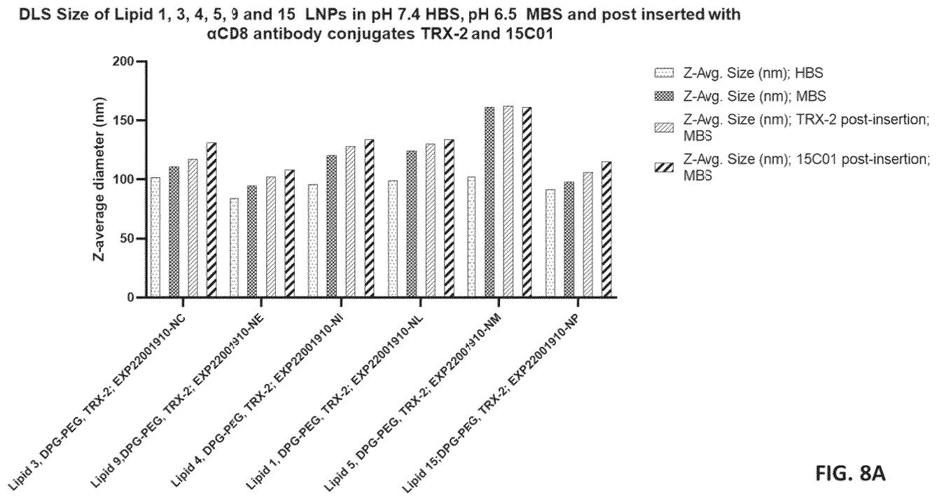

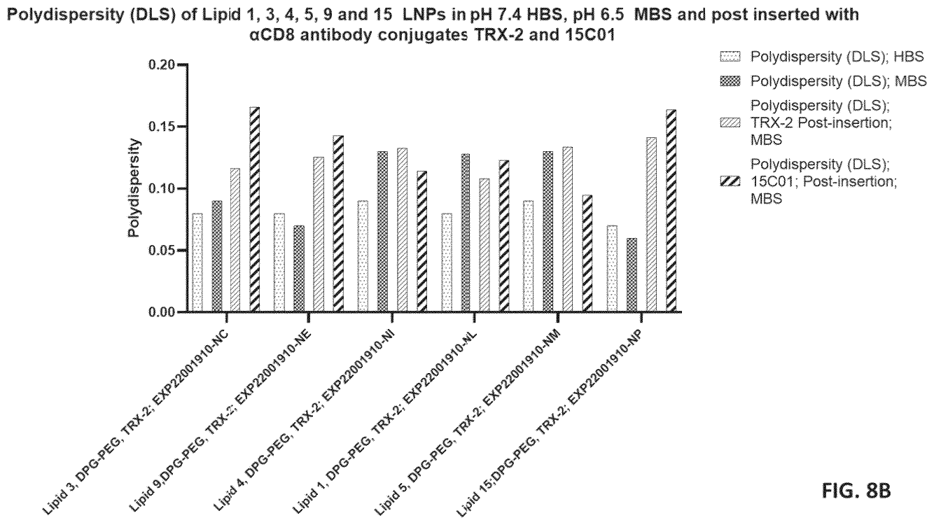

Lipids 1, 3, 4, 5, 9 and 15 LNPs encapsulating GFP-RNA (custom made by TriLink Biotechnologies Inc.) were prepared and characterized using methods described in Examples 5 to 8. Measured LNP size, and PDI, Lipids 1, 3, 4, 5, 9 and 15 LNPs in both pre- and post-insertion with αCD8 fab (TRX-1 and 15C01) conjugates are summarized in Table 19. As seen in FIG. 8A, the initial mixing step and subsequent buffer exchange into HBS resulted in LNP sizes≤120 nm with Lipids 3, 9 and 15, while Lipid 5 LNPs exhibited>150 nm diameter upon buffer exchange into pH 6.5 MBS. Lipids 9 and 15 LNPs exhibited the smallest sizes (≤120 nm) after both αCD8 antibody conjugate insertions (TRX-2 and 15C01) and post one freeze-thaw cycle. Similarly, polydispersity of all final αCD8 targeted LNPs remained<0.2 both pre- and post-freeze thaw (FIG. 8B). The ability of lipids 1, 3, 4, 5, 9 and 15 LNPs for inducing in vitro protein transfection in primary human T-cells mediated by αCD8 T-cell receptor targeting antibodies (TRX-2 and 15C01) was evaluated as described in Examples 18 and 19.

TABLE 19
Lipid 1, 3, 4, 5, 9, and 15 LNP size and PDI pre- and post- insertion with αCD8 (TRX-1 and 15C01) targeting Fab conjugate

| | | | Z-Avg. | Z-Avg. | | | | |
|---|---|---|---|---|---|---|---|---|
| | | | Size (nm); | Size (nm); | | | PDI (DLS); | PDI (DLS); |
| | Z-Avg. | Z-Avg. | TRX-2 | 15C01 | | | TRX-2 | 15C01; |
| Ionizable Lipid, | Size (nm); | Size (nm); | post-insertion; | post-insertion; | PDI (DLS); | PDI (DLS); | Post-insertion; | Post-insertion; |
| LNP no. | HBS | MBS | MBS | MBS | HBS | MBS | MBS | MBS |
| Lipid 3, DPG- | 101.5 | 110.8 | 117.4 | 131.0 | 0.08 | 0.09 | 0.116 | 0.166 |
| PEG, TRX-2; | | | | | | | | |
| EXP22001910- | | | | | | | | |
| NC | | | | | | | | |
| Lipid 9, DPG- | 83.9 | 94.8 | 102.5 | 108.1 | 0.08 | 0.07 | 0.1255 | 0.143 |
| PEG, TRX-2; | | | | | | | | |
| EXP22001910- | | | | | | | | |
| NE | | | | | | | | |
| Lipid 4, DPG- | 95.5 | 120.6 | 127.8 | 133.7 | 0.09 | 0.13 | 0.1325 | 0.114 |
| PEG, TRX-2; | | | | | | | | |
| EXP22001910- | | | | | | | | |
| NI | | | | | | | | |
| Lipid 1, DPG- | 98.78 | 124.25 | 130.2 | 133.7 | 0.08 | 0.128 | 0.108 | 0.123 |
| PEG, TRX-2; | | | | | | | | |
| EXP22001910- | | | | | | | | |
| NL | | | | | | | | |
| Lipid 5, DPG- | 102.4 | 161.2 | 162.3 | 161.3 | 0.09 | 0.13 | 0.1335 | 0.095 |
| PEG, TRX-2; | | | | | | | | |
| EXP22001910- | | | | | | | | |
| NM | | | | | | | | |
| Lipid 15; DPG- | 91.55 | 97.65 | 106.0 | 115.1 | 0.07 | 0.06 | 0.1415 | 0.164 |
| PEG, TRX-2; | | | | | | | | |
| EXP22001910- | | | | | | | | |
| NP | | | | | | | | |

### Example 13. Physiochemical Properties (Pre- and Post-Insertion with αCD8 (TRX-2) Fab Conjugate) of Lipids 1, 8, 9, 10, 11 and 15 GFP RNA Containing Lipid Nanoparticles (LNPs)

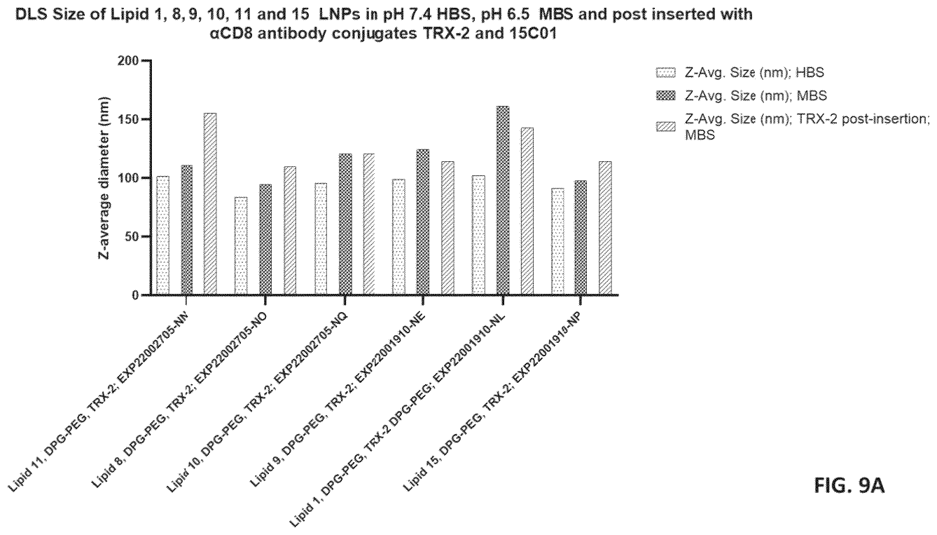

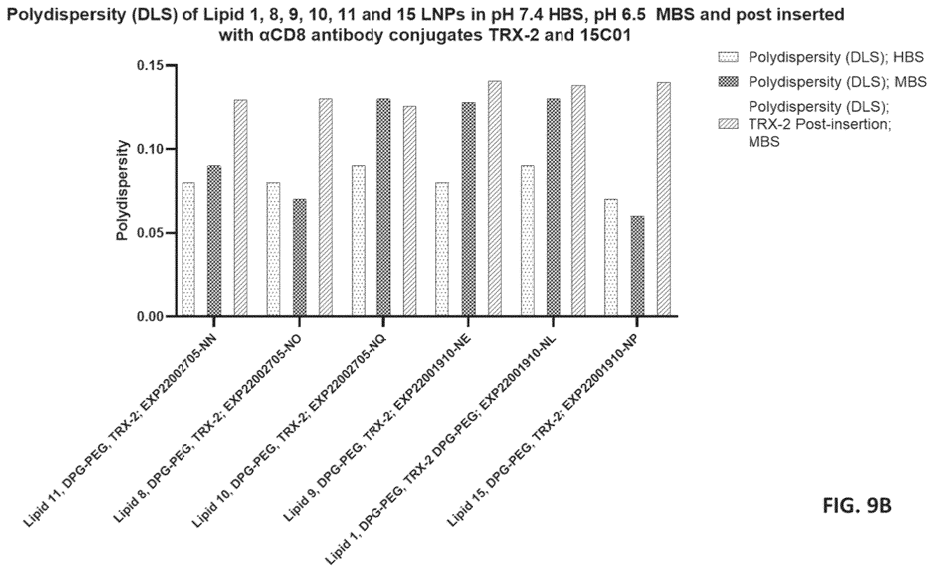

Lipids 1, 8, 9, 10, 11 and 15 LNPs encapsulating GFP-RNA (custom made by TriLink Biotechnologies Inc.) were prepared and characterized using methods described in Examples 5 to 8. Measured LNP size, and PDI, Lipids 1, 8, 9, 10, 11 and 15 LNPs in both pre- and post-insertion with αCD8 (TRX-2) Fab conjugate are summarized in Table 20. As seen in FIG. 9A, the initial mixing step and subsequent buffer exchange into HBS resulted in LNP sizes<130 nm with Lipids 8, 9, 10, 11 and 15, while Lipid 1 LNPs exhibited>150 nm diameter upon buffer exchange into pH 6.5 MBS. Lipids 8, 9, 10 and 15 LNPs exhibited the smallest sizes (≤130 nm) after both &CD8 antibody conjugate insertions (TRX-2 and 15C01) and post one freeze-thaw cycle. Polydispersity of all final αCD8 targeted LNPs remained<0.2 both pre- and post-freeze thaw (FIG. 9B). The ability of lipids 1, 8, 9, 10, 11 and 15 LNPs for inducing in vitro protein transfection in primary human T-cells mediated by αCD8 T-cell receptor targeting antibodies (TRX-2 and 15C01) was evaluated as described in Examples 16 and 17.

TABLE 20
Lipid 1, 8, 9, 10, 11 and 15 LNP size and PDI pre- and post-
insertion with αCD8 (TRX-2) targeting Fab conjugate

| | | | Z-Avg. | | | |
|---|---|---|---|---|---|---|
| | | | Size (nm); | | | PDI (DLS); |
| | Z-Avg. | Z-Avg. | TRX-2 post- | | | TRX-2 Post- |
| Ionizable Lipid, | Size (nm); | Size (nm); | insertion; | PDI (DLS); | PDI (DLS); | insertion; |
| LNP no. | HBS | MBS | MBS | HBS | MBS | MBS |
| Lipid 11, DPG-PEG, | 101.5 | 110.8 | 155.45 | 0.08 | 0.09 | 0.13 |
| TRX-2; EXP22002705- | | | | | | |
| NN | | | | | | |
| Lipid 8, DPG-PEG, | 83.9 | 94.8 | 110 | 0.08 | 0.07 | 0.13 |
| TRX-2; EXP22002705- | | | | | | |
| NO | | | | | | |
| Lipid 10, DPG-PEG, | 95.5 | 120.6 | 120.65 | 0.09 | 0.13 | 0.13 |
| TRX-2; EXP22002705- | | | | | | |
| NQ | | | | | | |
| Lipid 9, DPG-PEG, | 98.78 | 124.25 | 114.45 | 0.08 | 0.128 | 0.14 |
| TRX-2; EXP22001910- | | | | | | |
| NE | | | | | | |
| Lipid 1, DPG-PEG, | 102.4 | 161.2 | 142.75 | 0.09 | 0.13 | 0.14 |
| TRX-2 DPG-PEG; | | | | | | |
| EXP22001910-NL | | | | | | |
| Lipid 15, DPG-PEG, | 91.55 | 97.65 | 114.45 | 0.07 | 0.06 | 0.14 |
| TRX-2: EXP22001910- | | | | | | |
| NP | | | | | | |

### Example 14. Physiochemical Properties (Pre- and Post-Insertion with aCD8 Fab Conjugate T8) of Lipids 3, 4, 33, and 34 CAR(TTR-023) RNA Lipid Nanoparticles (LNPs)

Physiochemical properties (pre- and post-insertion with αCD8 Fab conjugate T8) of Lipids 3, 4, 33, and 34 CAR (TTR-023) RNA Lipid nanoparticles (LNPs)

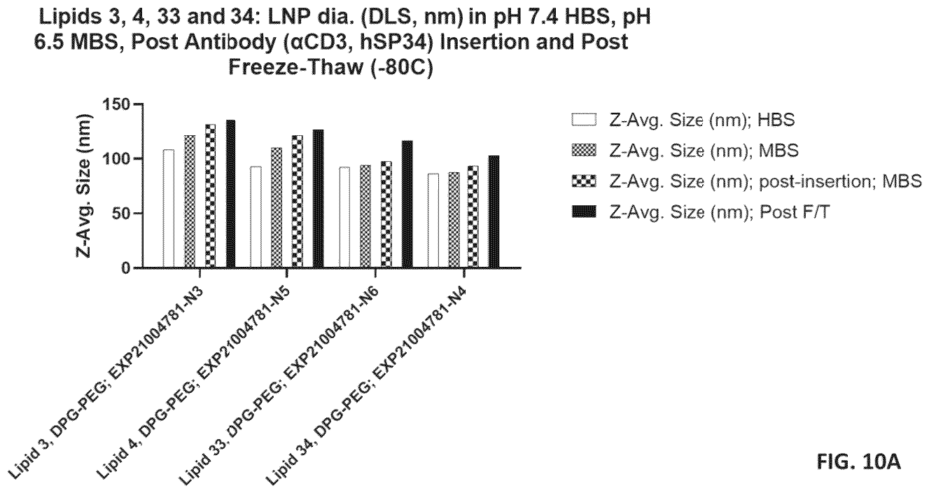

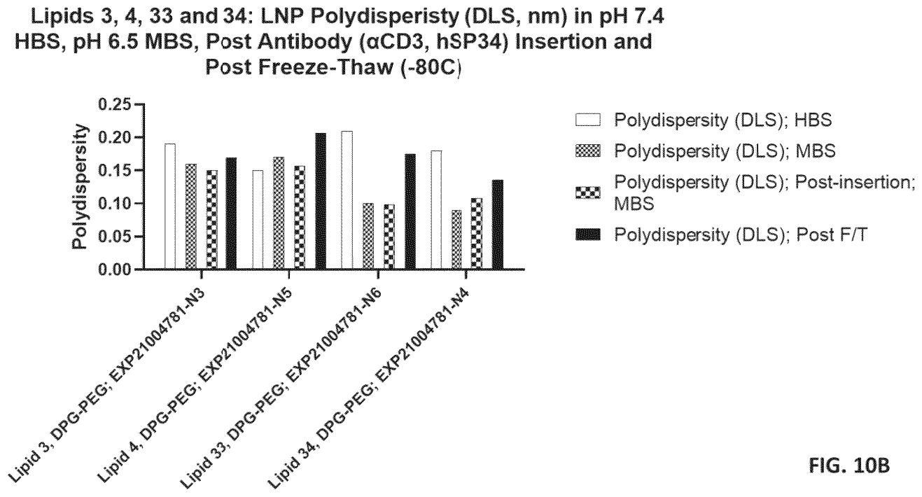

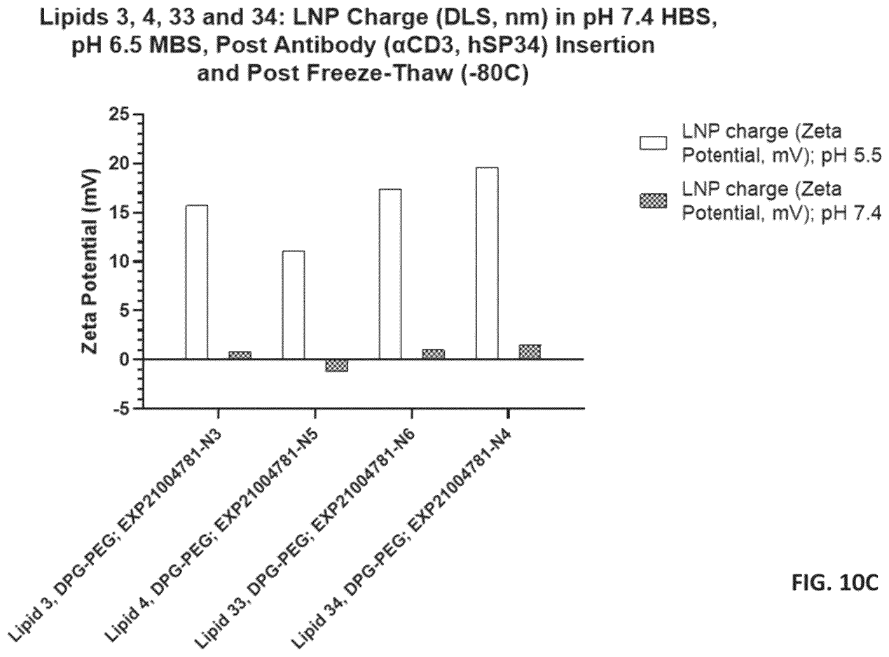

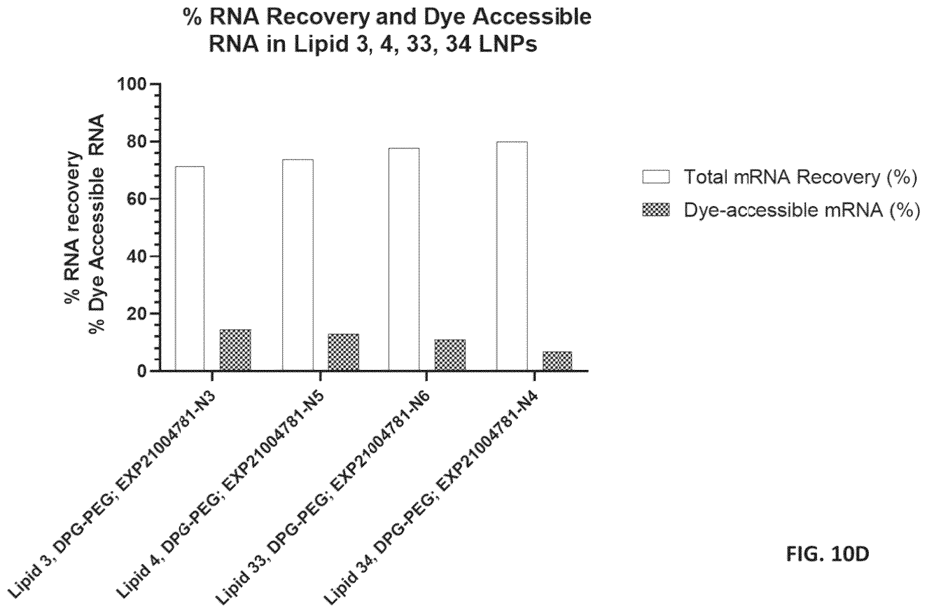

Lipids 3, 4, 33, and 34 LNPs encapsulating CAR (TTR-023) RNA (custom made by TriLink Biotechnologies Inc.) were prepared and characterized using methods described in Examples 5 to 8. Measured LNP size, PDI, charge and RNA content values of Lipids 3, 4, 33, and 34 LNPs are summarized in Table 21, Table 22 and Table 23. As seen in FIG. 10A, the initial mixing step and subsequent buffer exchanges into HBS and then MBS resulted in LNP sizes≤125 nm with Lipids 3 and 4, and ≤100 nm with Lipids 33 and 34. All LNPs exhibited moderate to high encapsulation efficiency (<15% Dye accessible RNA) and >70% RNA recovery, with Lipids 33 and 34 trending better in terms of recovery and lower in terms of dye accessible RNA (FIG. 10D). The subsequent buffer exchange into pH 6.5 MES buffer and antibody insertion process (37 C, 4 hours) resulted in Lipid 3 and 4 LNP diameters in the 120 nm to 135 nm range, while Lipids 33 and 34 yield sub-100 nm diameters. Similarly, Lipid 3 and 4 LNP PDI trended slightly higher in the 0.15 to 0.21 range, while Lipids 33 and 34 yield PDI≤0.11, indicating slightly better size distribution properties. Lipid 4 and 34 LNPs showed moderate size increases after one freeze-thaw cycle, however, a notably larger size shifts were observed with lipid 3 and 33 LNPs. In summary, all four lipids tested resulted in viable CAR mRNA encapsulation and freeze-thaw stability as well as <150 nm final targeted LNP diameters. The ability of lipids 3, 4, 33, and 34 LNPs for inducing in vitro CAR protein expression in primary human T-cells mediated by αCD3 or αCD8 T-cell receptor targeting was evaluated as described in Examples 16 and 17.

TABLE 21
Lipids 3, 4, 33, 34 LNPs Size, Polydispersity (DLS) data in pH 7.4 HBS,
pH 6.5 MBS and post αCD3 antibody Fab (hSP34) conjugate insertion

| | | | Z-Avg. | | | | Polydispersity | |
|---|---|---|---|---|---|---|---|---|
| | Z-Avg. | Z-Avg. | Size (nm); | Z-Avg. | Polydispersity | Polydispersity | (DLS); | Polydispersity |
| Ionizable lipid, | Size (nm); | Size (nm); | post-insertion; | Size (nm); | (DLS); | (DLS); | Post-insertion; | (DLS); |
| LNP no. | HBS | MBS | MBS | Post F/T | HBS | MBS | MBS | Post F/T |
| Lipid 3, | 108.6 | 121.5 | 131.8 | 135.5 | 0.19 | 0.16 | 0.15 | 0.17 |
| DPG-PEG; | | | | | | | | |
| EXP21004781- | | | | | | | | |
| N3 | | | | | | | | |
| Lipid 4, | 92.8 | 110.2 | 121.7 | 127.1 | 0.15 | 0.17 | 0.16 | 0.21 |
| DPG-PEG; | | | | | | | | |
| EXP21004781- | | | | | | | | |
| N5 | | | | | | | | |
| Lipid 33, | 92.2 | 94.1 | 97.5 | 116.9 | 0.21 | 0.1 | 0.10 | 0.18 |
| DPG-PEG; | | | | | | | | |
| EXP21004781- | | | | | | | | |
| N6 | | | | | | | | |
| Lipid 34, | 86.3 | 87.8 | 93.4 | 103 | 0.18 | 0.09 | 0.11 | 0.14 |
| DPG-PEG; | | | | | | | | |
| EXP21004781- | | | | | | | | |
| N4 | | | | | | | | |

TABLE 22
Lipids 3, 4, 33, 34 LNP charge, Zeta Potential,
ZP (DLS, mV) in pH 5.5 MES and pH 7.4 HBS

| | Charge | Charge |
|---|---|---|
| | (ZP, mV); | (ZP, mV); |
| Ionizable lipid, LNP no. | pH 5.5 | pH 7.4 |
| Lipid 3, DPG-PEG; EXP21004781-N3 | 15.7 | 0.786 |
| Lipid 4, DPG-PEG; EXP21004781-N5 | 11.1 | −1.23 |
| Lipid 33, DPG-PEG; EXP21004781-N6 | 17.4 | 1.01 |
| Lipid 34, DPG-PEG; EXP21004781-N4 | 19.6 | 1.5 |

TABLE 23
Lipids 3, 4, 33, 34 LNP Dye Accessible RNA and total RNA content

| | Nominal | Measured | Dye- | Total | Dye- |
|---|---|---|---|---|---|
| | mRNA | Total | accessible | mRNA | accessible |
| Ionizable lipid, | Conc. | mRNA | mRNA | Recovery | mRNA |
| LNP no. | (μg/mL) | (μg/mL) | (μg/mL) | (%) | (%) |
| Lipid 3, DPG-PEG; | 150 | 107.1 | 15.5 | 71.4 | 14.5 |
| EXP21004781-N3 | | | | | |
| Lipid 4, DPG-PEG; | 150 | 110.7 | 14.4 | 73.8 | 13.0 |
| EXP21004781-N5 | | | | | |
| Lipid 33, DPG-PEG; | 150 | 116.4 | 12.8 | 77.6 | 11.0 |
| EXP21004781-N6 | | | | | |
| Lipid 34, DPG-PEG; | 150 | 119.9 | 8.2 | 79.9 | 6.8 |
| EXP21004781-N4 | | | | | |

### Example 15. Method for Freezing (and Thaw) Process for LNP Suspension and LNP Characterization Post Freeze-Thaw

LNP suspension was mixed with a solution of 49 wt % sucrose solution in water and additional storage buffer (if needed) to achieve a final sample containing LNPs at approximately 45 μg/mL and sucrose at approximately 9.6 wt %. Aliquots of approximately 0.05 mL in 1.5 mL centrifuge tubes were then prepared from the final LNP sample containing sucrose. The aliquots were then placed in a −80° C. freezer for at least 2 h to freeze the samples. After freezing, an aliquot was thawed by placing it at room temperature for at least 10 min. The aliquot was then mixed by vortexing at 2500 rpm for approximately 5 s. The thawed material was then analyzed for size by DLS as described in Example 3.

### Example 16. Method for the Primary Human T-Cell Transfection with DiI-C18-5DS Labelled LNPs

CD3+ T cells were isolated from frozen peripheral blood mononuclear cells using an EasySep Human T Cell Isolation Kit on a RoboSep automated cell isolation system from STEMCELL. T cells were plated into a round bottom 96-well plate in RPMI cell culture media supplemented with glutamax, 10% fetal bovine serum, pen-strep, and 40 ng/ml IL-2. 100 μL of cell suspension was seeded per well at a density of 1M T cells/mL (100K T cells/well). Cells were allowed to rest for two hours in a 37° C. incubator, and then were transfected by gently adding 10 μL of a 22 μg/mL (by mRNA) nanoparticle suspension, resulting in a final mRNA concentration of 2 μg/mL (unless otherwise noted). Cells were gently mixed with a pipette and then incubated for 24 hours in a 37° C. incubator. After incubation the cells were diluted with FACS buffer (BD 554657) and analyzed using a BD Fortessa flow cytometer. Data were analyzed using FlowJo software from BD biosciences.

### Example 17. CD4 AND CD69 Staining Protocol Post LNP Transfection

After 24 hours, cells were transferred to a 96-well conical bottom polypropylene plate and centrifuged at 350×g for 5 minutes. Supernatants were removed and transferred to a fresh conical bottom polypropylene plate for further analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100× by adding 100 μL of each antibody to 10 mL FACS buffer. 100 μL of the diluted antibody solution was added to each well and the plate was incubated at room temperature for 20 minutes. The plates were then washed by centrifuging at 350×g for 5 min, removing the supernatant, re-suspending in 200 μL FACS buffer, centrifuging at 350×g for 5 min and aspirating the supernatant from each well. Following the wash, cells were resuspended in 100 μL of 1.6% formaldehyde and stored at 4° C. until FACS analysis. FACS analysis was performed using a BD Fortessa equipped with a High Throughput Sampler.

### Example 18. Human IFN-T ELISA Method

IFN-γ was assayed using an R&D Duoset IL-2 ELISA kit, PN DY285B. Briefly: an Immulon 2HB 96-well plate (Thermo X1506319) was coated by adding 100 μL of a 2 μg/mL solution of the R&D IL-2 capture antibody to each well and then incubating the plate overnight at 4° C. The plate was washed three times with wash buffer (0.05 TWEEN-20 in pH 7.4 TRIS buffered saline, Thermo 28360), blocked with reagent diluent (0.1% BSA in wash buffer) for one hour at room temperature, and then washed an additional three times with wash buffer. Supernatants were diluted three-fold in reagent diluent and then 100 μL of diluted supernatant was added to each well. IFN-γ standards were prepared on the same plate by serial dilution. Plates were incubated for two hours at room temperature, washed three times with wash buffer. 100 μL of detection antibody diluted in reagent diluent was added, incubated for 2 hours at room temperature, and then the plate was washed three times with wash buffer. 100 μL of Streptavidin-HRP was added, incubated for 20 minutes at room temperature, and then the plate was washed three times with wash buffer. 100 μL of substrate solution (Thermo N301) was added, incubated for 20 minutes at room temperature and then the reaction was quenched by adding 100 μL of stop solution (Invitrogen SS04). Optical density at 450 nm was read on a SpectraMax M5 plate reader. IFN-γ concentration was quantified relative to a standard curve based on contemporaneously analyzed IFN-γ standards.

### Example 19. TTR-023 CAR (M1) Staining Protocol

Cells were transferred to a 96-well conical bottom plate and washed by centrifuging at 350×g for 5 minutes followed by resuspension in FACS buffer (BD 554657). TTR-023 CAR protein, which contains a FLAG tag, was detected by staining with M1 anti-Flag antibody (Sigma Aldrich F3040) conjugated to Alexa Fluor 488 (Invitrogen A3750) at room temperature for 20 minutes. Following staining, cells were washed twice by centrifugation at 350×g for 5 minutes followed by resuspension in FACS buffer. Cells were analyzed by FACS using a BD Fortessa flow cytometer (BD Biosciences).

Synthesis and purification procedure for the M1 anti-Flag antibody—Alexa Fluor 488 conjugate: M1 anti-FLAG antibody was buffer exchanged into pH 8.3 sodium bicarbonate buffer using a Zeba 40K MWCO spin column. An 8 mg/mL solution of Alexa Fluor 488 TFP ester (Invitrogen A37570) in DMSO was then added to a final AB: dye mass ratio of 10:1 and the mixture was allowed to react for 1 hour with gentle shaking. Fluorophore conjugated M1 was purified and buffer exchanged into pH 7.4 PBS by passing the reaction mixture through two sequential Zeba spin columns. Protein content and degree of labeling were assessed by UV-Vis spectroscopy.

### Example 20. In Vitro LNP Car Primary Human T-Cell Transfection and Raji (B-Cell) Co-Culture for Assessment of T-Cell Function

CD3+ or CD8+ T cells were isolated from human PBMCs by using EasySep Human CD8+ T cell isolation kit (catalog #17953) and EasySep Human T cell isolation kit (catalog #17951) according to manufacturer's instructions. Isolated T cells were rested overnight in T cell media (RPMI+Glutamax, Gibco, catalog #61870-036) with 10% heat inactivated fetal bovine serum (HI—FBS, Gibco, catalog #16140-071) supplemented with 100 IU human IL-2 (Miltenyi catalog #130-097-748) at a density of 500,000 cells/well in a 96 well plate. T cells were then transfected with LNPs at a mRNA concentration of 2 μg/ml. CAR expression was detected 18 h post LNP transfection by using flow cytometry. Before co-culture with Raji B-cells, the transfected T cells were washed twice with T cell media. Raji B-cells were stained with cell trace violet (CTV, Thermo Fisher, catalog #C34571) and co-cultured with different effector to target (E:T) ratios of T cells in a 96 well plate format and incubated at 37° C. for 72 hours. 72 hours post co-culture the cells were stained with live/dead stain (eBioscience Fixable viability dye eFluor 780, Invitrogen catalog #2290917) and analyzed by flow cytometry. Data was analyzed with FlowJo and Graph Pad prism.

### Example 21. In Vitro Experimental Protocol for Whole Blood Transfection

This Example describes the method used to transfect immune cells in whole blood using Fab targeted mRNA LNPs.

Venous blood from healthy volunteers was anti-coagulated in heparin tubes (BD Biosciences #367526) and seeded at 50 μL in a 96-well round-bottom plate. Transfection of whole blood was carried out simply by adding nanoparticles containing 5 μg/mL mRNA to the cells and co-culturing at 37° C. until the time of analysis. To assess transfection efficiency, cells were analyzed 24-hours post-transfection by flow cytometry. LNPs used (with and without post-inserted targets) at 2.5 μg/mL: RDM073.23. Cells obtained from human blood were analyzed by flow cytometry. Prior to the analysis of whole blood transfection efficiency, red blood cells were lysed twice with VersaLyse Lysing Solution (Beckman Coulter #A09777) for 10 minutes at room temperature. Primary antibodies applied in the flow cytometry analysis of whole blood included the following: CD4-FITC (1:200) (BD Biosciences #555346), CD19-BUV395 (1:400) (BD Biosciences #563551), CD56-BUV737 (1:400) (BD Biosciences #741842). Fixable Viability Dye eFluor780 (eBiosciences #65-0865-14) was used to assess viability for all samples. For flow analysis, 1×10⁵ cells were Fc-blocked (BD Biosciences #564219) for 5 minutes on ice, followed by labeling dead cells with fixable viability dye eFluor780 and surface staining for 30 minutes on ice with specific antibodies.

Compensation for each fluorochrome was performed in the multicolor flow panels using positive and negative compensation beads. Fluorescence minus one (FMO) samples and unstained controls were included to determine the level of background fluorescence and to set the gates for the negative cell populations versus the positive cell populations.

All samples were acquired on a BD LSRFortessa X-20 (BD Biosciences) running FACSDIVA software (Becton Dickinson). All data collected were analyzed using FlowJo 10.7.1 software and GraphPad Prism version 9.0.

### Example 22. Protocol for In Vivo T-Cell Reprogramming (Using GFP Reporter Protein) in Human T-Cell Engrafted NSG-Mice

The following is a standard procedure for in-vivo reprogramming of immune cells with DiI LNP expressing GFP.

### Mice Strains and Humanization

6-8 weeks old NSG male mice were engrafted with 10 million PBMC of qualified donor in sterile PBS by tail vein injection. Individual body weight was monitored twice a week and blood samples were collected at appropriate interval to evaluate human immune cells engraftment.

### Evaluation of Human T-Cell Engraftment in the Immunodeficient Mice

50 μl blood was collected by tail vein bleed from each mouse. Red blood cells were lysed using Versalyse, RBC lysis solution following protocol as instructed by manufacturer (Beckman Coulter A09777). Cells were stained with hCD45 & hCD3 to determine the engraftment of human T-cells. After 15 days of PBMC injection, mice had anywhere from 30-60% huCD45+. These humanized mice were evaluated for reprogramming of immune cells by LNPs expressing Dil dye and GFP.

### Reprogramming of Immune Cells

At time zero, mice (n=4 per group) were injected with GFP expressing DiI LNPs (by i.v. with appropriate buffer). At each time point, 24 or 48 h depending on the example mice treated with either LNPs or buffer were sacrificed. Terminal blood and tissues collection was performed to determine Dil and GFP expression in different organs and immune cells as below.

### Tissue and Blood Sample Collection.

At above specified timepoints, mice were anesthetized with CO₂ before sample collection. For blood collection, the chest was opened to expose the heart. Up to 300 μl blood was drawn from the left ventricle and dispensed into a K3EDTA mini collect tube (Greiner Bio-One). Then a new syringe was used to draw remaining blood from the heart as much as possible. All the immune organs; spleen, bone marrow was isolated along with liver and lung. Immune cells were isolated from spleen, via smearing and shredding it through syringe and cell suspension was filtered through 70 μM cell strainer and was washed with PBS. Bone marrow was flushed with needle to collect all the immune cells. A piece of liver and lung tissue was gently grinded with tissue homogenizer and the homogenized and cells were isolated using Miltenyi liver dissociation kit, (Miltenyi Biotec, Catalog #130-105−807) and lung dissociation kit (Miltenyi Biotec, Catalog #130-0950927) and instruction were followed according to the manufacturing instruction.

### Immunophenotyping Analysis

Immune cells from blood and all the above organs were processed with Versalyse, RBC lysis buffer as per manufacturing instructions. Immune cells were stained with live/dead fixable dye and surface markers with standard flow analysis protocol as shown in Table 24. BD symphony flow cytometer was used to determine positive population.

TABLE 24

| Antigen | Fluorophore | Clone | Company | Catalog# |
|---|---|---|---|---|
| DiI | APC | NA | NA | NA |
| GFP | mRNA | NA | NA | NA |
| Anti-CD45 | BUV395 | HI30 | BD Biosciences | 563792 |
| Anti-CD3 | BUV805 | UCHT1 | BD Biosciences | 612895 |
| Anti-CD4 | BV711 | SK3 | BioLegend | 344648 |
| Anti-CD8 | BV421 | RPA-TB | BD Biosciences | 562428 |
| Anti-CD45 | BB700 | 30-F11 | BD Biosciences | 566439 |
| Anti-CD11b | BV785 | M1/70 | BioLegend | 101243 |
| Anti-F4/80 | PE Dazzle | BM8 | BioLegend | 123146 |
| Anti-CD31 | BUV737 | MEC 13.3 | BD Biosciences | 612802 |
| TruStain Monocyte | NA | NA | BioLegend | 462103 |
| Blocker | | | | |
| Arc Amine Comp | NA | NA | NA | 01-3333-42 |
| beads | | | | |
| UltraComp eBeads | NA | NA | Invitrogen | 01-3333-42 |
| LIVE/DEAD Far | NA | NA | Invitrogen | L34974 |
| Red Stain | | | | |
| TruStain Fc X | NA | NA | Invitrogen | 422302 |

### Example 23. In Vitro Protein Expression (GFP) in Primary Human T-Cells of Lipids 3, 6, 7 and Comparator Lipid ACD3 Targeted LNPs (DLin-MC3-DMA and ALC-0315) Stored at 4° C. and after One Freeze-Thaw Cycle (Post −80° C. Storage)

This example compares the GFP protein expression resulting from LNP's derived from Lipids 3, 6 and 7 to LNPs made using comparator lipids DLin-MC3-DMA and ALC-0315. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression.

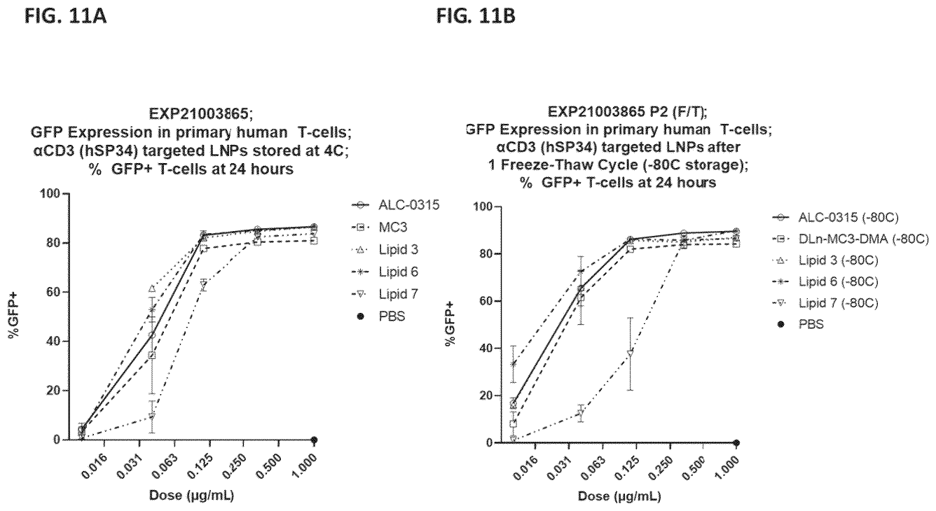

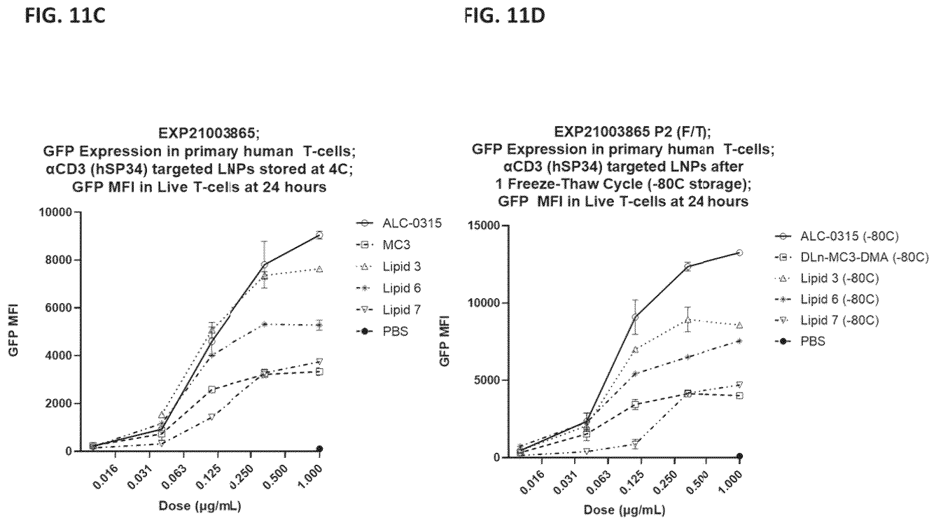

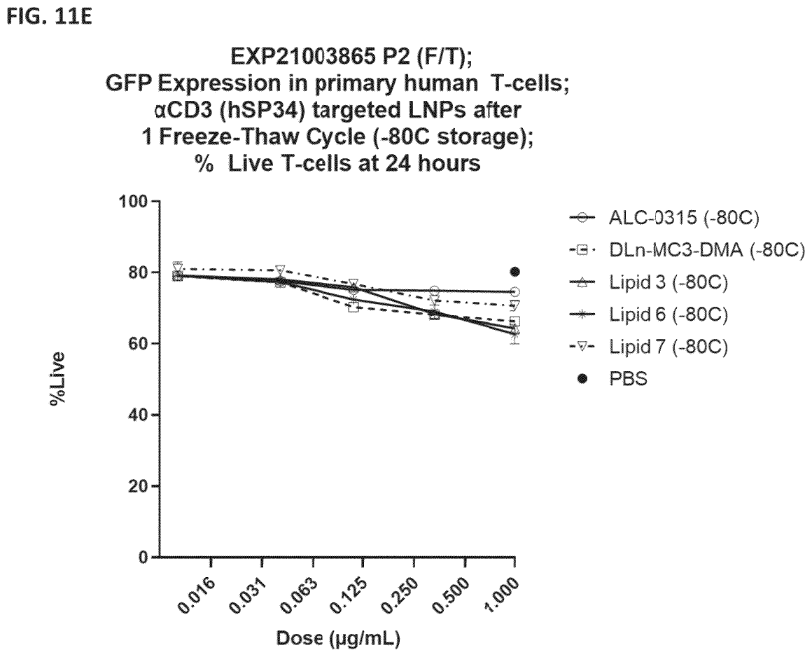

Lipid 3 and ALC-0315 LNPs resulted in similar GFP protein expression levels as indicated by similar % GFP+ T-cells and similar Mean Fluorescence Intensity (MFI) of the transfected T-cells (see FIG. 11A, FIG. 11B (% GFP+) and FIG. 11C and FIG. 11D (GFP MFI). This suggests that the antibody driven uptake was equally effective with both ionizable lipids and similar levels of ionizable lipid driven endosomal escape efficiencies were achieved. Lipid 7 and DLin-MC3-DMA lipids resulted in similar and lowest levels of GFP protein expression. Lipid 6 LNPs exhibited an intermediate level of GFP protein expression. The protein expression levels suggest that 9 carbon N-acyl substituent (lipid 3) enhances performance over an 11 carbon N-acyl substituents (of Lipids 6 and 7). The performance levels and relative order of LNP efficiency was preserved in all lipid nanoparticle formulation tested after 1 Freeze Thaw cycle (Post −80° C. storage) as illustrated by comparison of % GFP+ cells and GFP MFI values before and after −80° C. storage (FIG. 11A verses FIG. 11B and FIG. 11C versus FIG. 11D). While all lipid formulation tested were well tolerated by primary human T-cells (FIG. 11E); some dose dependent loss in T-cell viability was observed with lipids 7 and DLin-MC3-DMA.

### Example 24. In Vitro Protein Expression (GFP) in Primary Human T-Cells of Lipids 3, 4 and Comparator Lipid aCD3 Targeted LNPs (DLin-KC2-DMA and SM-102) Stored at 4° C. And after 1 Freeze-Thaw Cycle (Post −80° C. Storage)

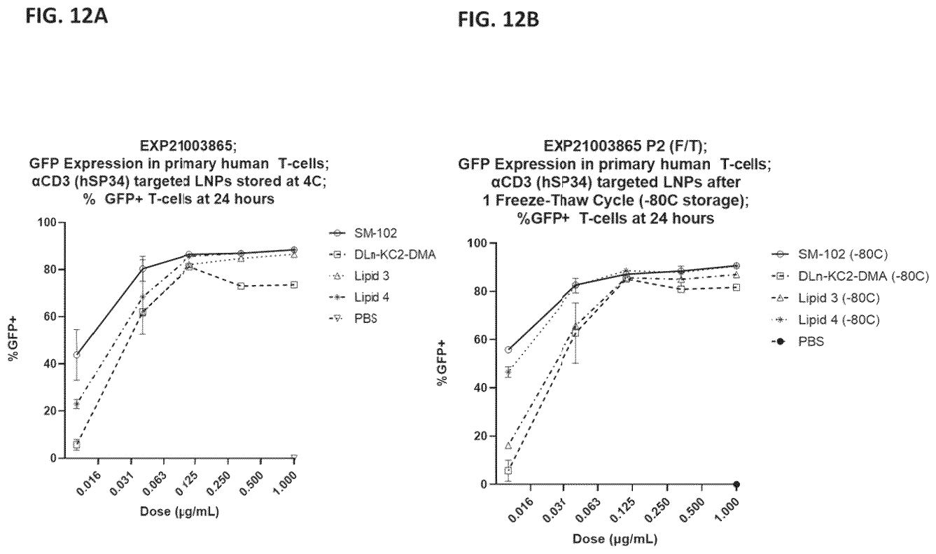

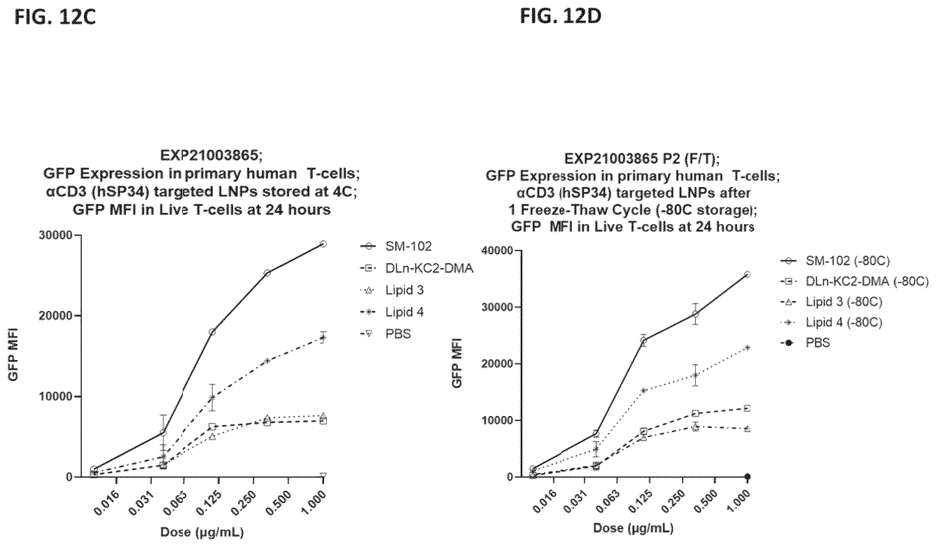

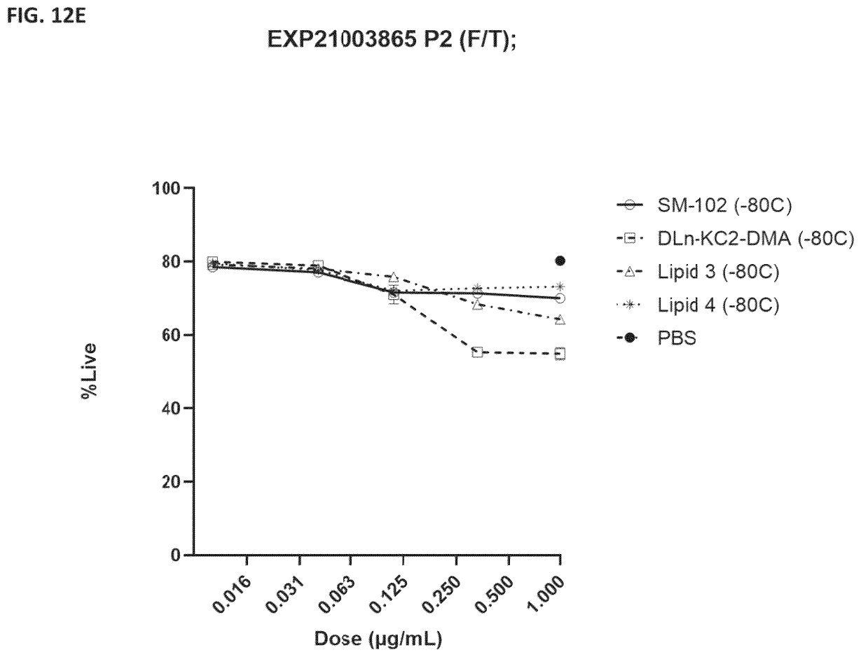

This example compares the GFP protein expression resulting from LNP's derived from Lipids 3 and 4 to LNPs made using comparator lipids DLin-KC2-DMA and SM-102. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At ≥0.1 μg/mL dose, similar number of T-cells were transfected with SM-102 and Lipid 4 LNPs, however, at doses below 0.1 μg/mL, a larger fraction of T-cells were GFP+ with counted with SM-102 LNPs and the GFP MFI values were consistently about 2-fold higher at all doses suggesting more efficient cytosolic access/cytosolic release of encapsulated GFP mRNA relative to Lipid 4 LNPs. Lipid 3 and DLin-KC2-DMA LNPs performed similarly in terms of the fraction of T-cells accessed as well as copies of GFP protein expressed on a per cell basis and were at levels by 2-fold lower relative to Lipid 4 LNPs. This trend indicates that the oleoyl tail groups of Lipid 4 resulted in more efficient cytosolic access/cytosolic release of encapsulated GFP mRNA relative to the linoleoyl tail groups of Lipid 3. The performance levels and relative order of LNP efficiency was well preserved in all lipid nanoparticle formulation tested after 1 Freeze Thaw cycle (Post-80° C. storage) as illustrated by comparison of % GFP+ cells and GFP MFI values before and after −80° C. storage (FIG. 12A verses FIG. 12B and FIG. 12C versus FIG. 12D). Lipids 3, 4, and SM-102 LNPs were well tolerated by primary human T-cells (FIG. 12E); however, a slightly greater loss in T-cell viability was observed with DLin-KC2-DMA LNPs at the higher doses of 0.3 and 1 μg/mL.

### Example 25. In Vitro Protein Expression (GFP) in Primary Human T-Cells of Lipids 1, 3, 8 and Comparator Lipid ACD3 Targeted LNPs (DLin-KC2-DMA) Stored at 4° C. and after 1 Freeze-Thaw Cycle (Post −80° C. Storage)

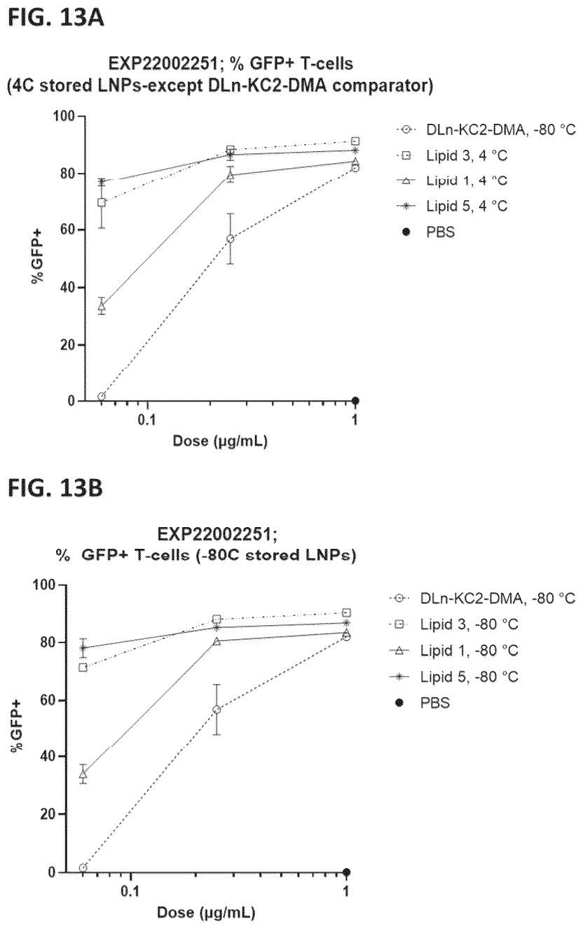

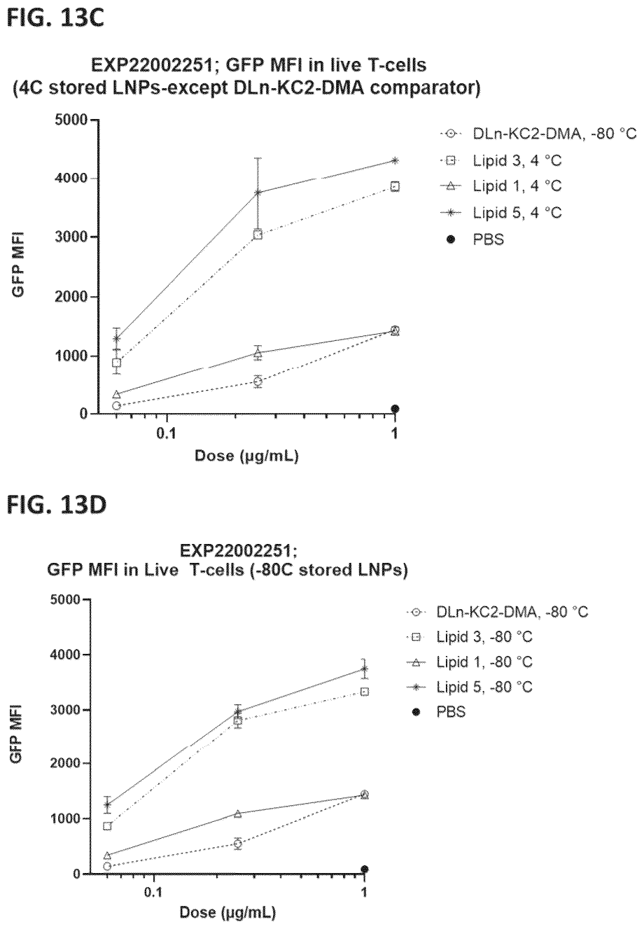

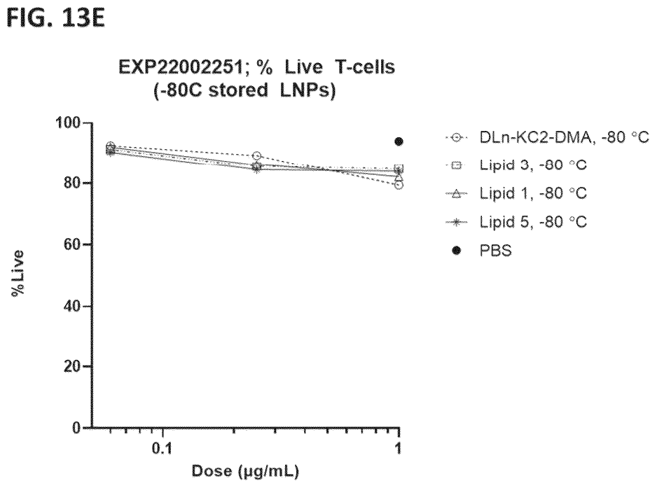

This example compares the GFP protein expression resulting from LNP's derived from Lipids 1, 3 and 5 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, Lipid 1 and DLin-KC2-DMA LNPs performed similarly both in terms of fraction of GFP+ T-cells (reflected by % GFP+ cells) and the copies of GFP protein produced on a per cell basis (reflected by GFP MFI values). Lipid 3 LNPs resulted in a roughly 3-fold higher fraction of GFP+ T-cells relative to Lipid 1 LNPs and the GFP MFI values were consistently about 3-fold higher at all doses suggesting more efficient cytosolic access/cytosolic release of encapsulated GFP mRNA with Lipid 3 LNPs relative to Lipid 1 indicating that the 9 carbon N-acyl substituent (of Lipid 3) is favorable for efficient cytosolic access/cytosolic release of encapsulated GFP mRNA relative to the 10 carbon N-acyl substituent (of Lipid 1). Highest GFP protein expression was observed with Lipid 5 LNPs as indicated by the higher % GFP+ and GFP MFI values at all dose levels. This indicates that further reduction in carbon number of the N-acyl substituent to 8 carbon skeleton further improves the mRNA delivery efficiency. However, as noted in Example 9, Lipid 5 LNPs exhibited larger size shifts in the buffer exchange and antibody conjugate insertion steps resulting in final LNP hydrodynamic diameters>150 nm indicating that 9-carbon N-acyl substitution skeleton proves optimal for LNP size (for enabling 0.2 micron sterile filtration) as well as the ability to induce reporter gene expression. The performance levels and relative order of LNP efficiency was well preserved in all formulation tested after 1 Freeze Thaw cycle (Post −80° C. storage) as illustrated by comparison of % GFP+ cells and GFP MFI values before and after −80° C. storage (FIG. 13A verses FIG. 13B and FIG. 13C versus FIG. 13D). Lipids 1, 3, 5 LNPs were well tolerated by primary human T-cells (FIG. 13E).

### Example 26. In Vitro Protein Expression (GFP) in Primary Human T-Cells of Lipids 1, 8 and Comparator Lipid aCD3 Targeted LNPs (DLin-KC2-DMA) Stored at 4° C. And after 1 Freeze-Thaw Cycle (Post −80° C. Storage)

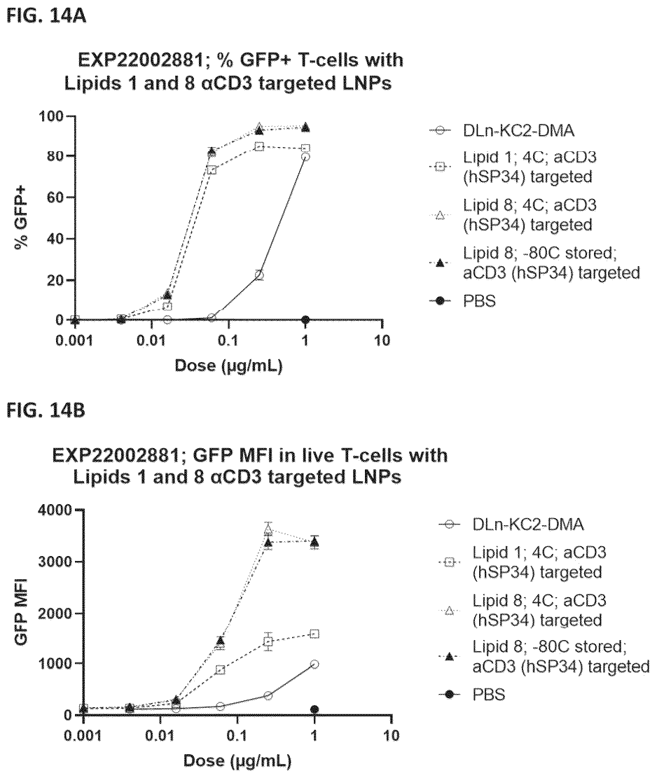

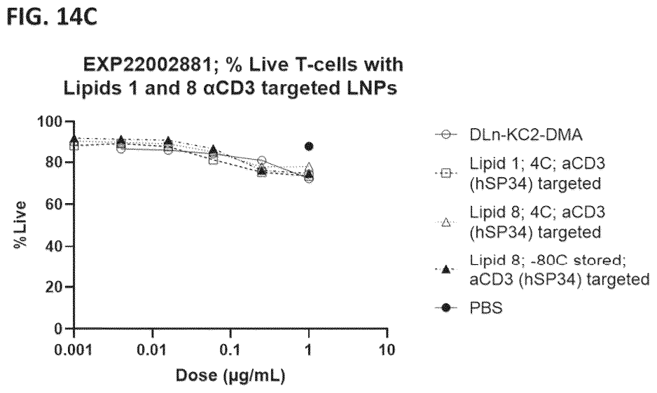

This example compares the GFP protein expression resulting from LNP's derived from Lipids 1 and 8. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, both Lipid 1 and LNPs resulted in similar fraction of GFP+ T-cells (reflected by % GFP+ cells), however, Lipid 8 LNPs out-performed Lipid 1 LNPs by a factor of 3 in terms of copies of GFP protein produced on a per cell basis (reflected by GFP MFI values). This indicates that the β-ethyl octanoyl N-acyl substituent (of Lipid 8) improves cytosolic access/cytosolic release of encapsulated GFP mRNA relative to the α-ethyl octanoyl N-acyl substituent (of Lipid 1). The overall effect of the β-ethyl substitution is better LNP size distribution properties (relative to Lipid 1 as illustrated in Example 9) and gain in mRNA delivery efficiency resulting in a 2-fold improvement in reporter gene expression levels despite the 10 carbon N-acyl substitution pattern. This indicates that both LNP properties and delivery efficiency are tunable via size (carbon number) and geometry (β-ethyl octanoyl versus α-ethyl octanoyl) of the N-acyl substituent. The performance of Lipid 8 LNP's was well preserved after 1 Freeze Thaw cycle (Post −80° C. storage) as illustrated by comparison of % GFP+ cells and GFP MFI values before and after −80° C. storage (FIG. 14A and FIG. 14B). Both Lipids 1 and 8 LNPs were well tolerated by primary human T-cells (FIG. 14C).

### Example 27. In Vitro Protein Expression (GFP) in Primary Human T-Cells of Lipids 8, 9, 10 and Comparator Lipid aCD3 Targeted LNPs (DLin-KC3-DMA) Stored at 4° C. and after 1 Freeze-Thaw Cycle (Post −80° C. Storage)

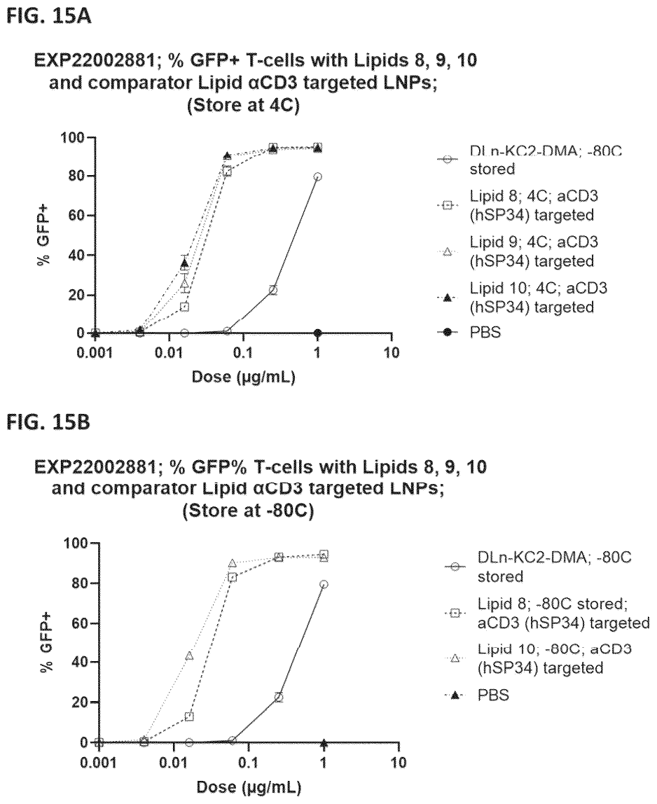

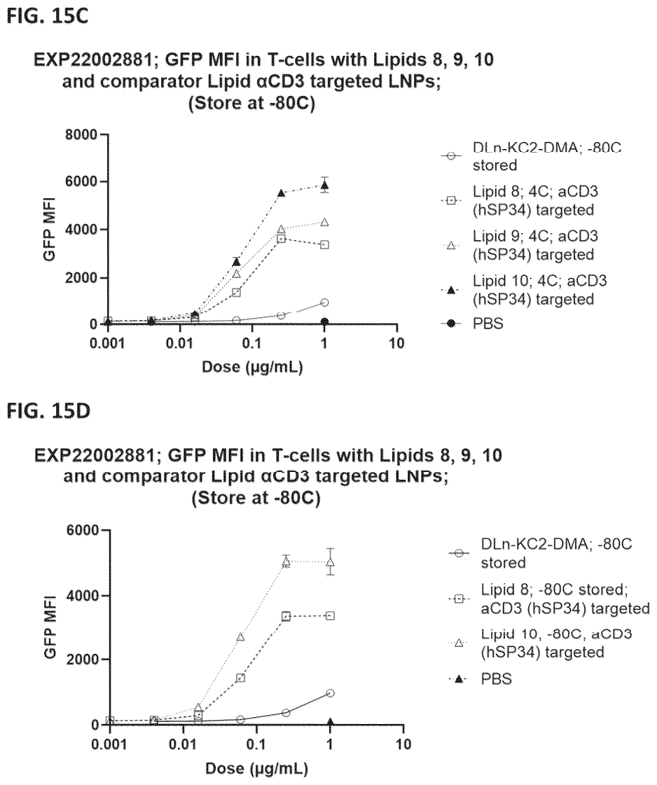

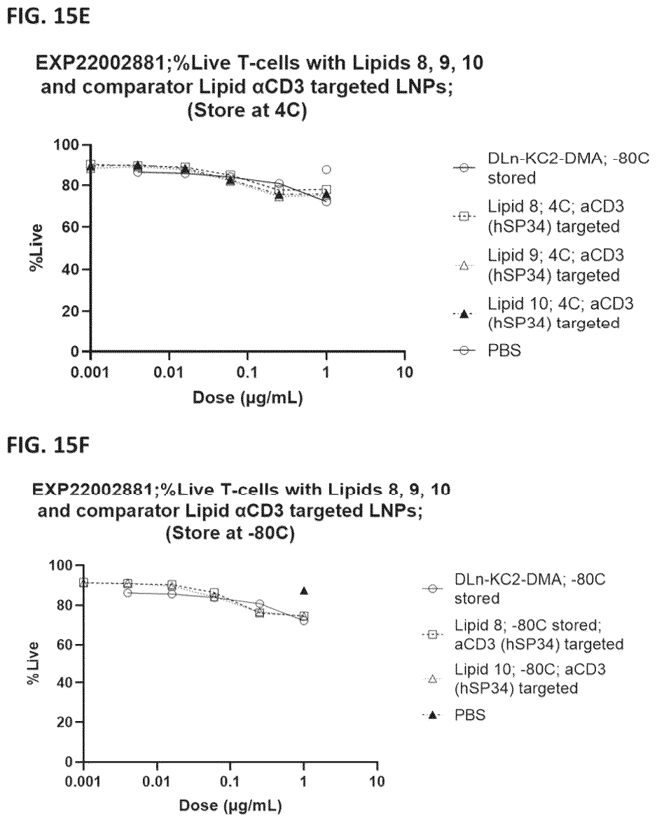

This example compares the GFP protein expression resulting from LNP's derived from Lipids 8, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. As shown in FIGS. 15A and 15C, at all dose levels, Lipid 8 and 9 LNPs performed similarly both in terms of fraction of GFP+ T-cells (reflected by % GFP+ cells) and the copies of GFP protein produced on a per cell basis (reflected by GFP MFI values). Thus, the succinic acid derived 14 carbon N-acyl substituent (in Lipid 9) results in similar efficiency of cytosolic access/cytosolic release of the mRNA payload relative to the 10 carbon N-acyl substituent (of Lipid 8) indicating that introduction of a carboxylate ester (and added polarity of the O-atoms) in N-acyl substituent balances and counters the loss of lipid efficiency seen with Lipids 6 and 7 (that both feature a 11 carbon N-acyl substituent). As illustrated in Example 10, Lipid 9 LNPs exhibit size distribution properties comparable to those of Lipids 6, 7 and 8 suggesting that biodegradable ester linkages (as in Lipid 9) can be introduced into the N-acyl substituent without loss of LNP size distribution properties. Lipid 10 LNPs out-performed Lipid 9 LNPs both in terms of fraction of GFP+ T-cells (reflected by % GFP+ cells) and the copies of GFP protein produced on a per cell basis (reflected by GFP MFI values) by a factor of 1.5. Suggesting that reduction in the total carbon count to 12 (in lipid 10) versus 14 (in Lipid 9) further improved lipid efficiency. Thus, analogous to trends observed with Lipids 3, 5, 6, 7 and 8 LNPs, the activity of succinic acid derived biodegradable N-acyl substituents in Lipids 9 and 10 is also tunable. The performance of Lipid 10 LNPs was well preserved after 1 Freeze Thaw cycle (Post −80° C. storage) as illustrated by comparison of % GFP+ cells and GFP MFI values before and after-80° C. storage (FIG. 15A verses FIG. 15B and FIG. 15C versus FIG. 15D). Lipids 9 and 10 LNPs were well tolerated by primary human T-cells (FIG. 15E and FIG. 15F).

### Example 28. In Vitro Protein Expression (GFP) in Primary Human T-Cells of Lipids 3, 4, 9, 15 and Comparator Lipid (DLin-KC2-DMA) aCD3 Targeted LNPs Stored at 4° C. and after 1 Freeze-Thaw Cycle (Post −80° C. Storage)

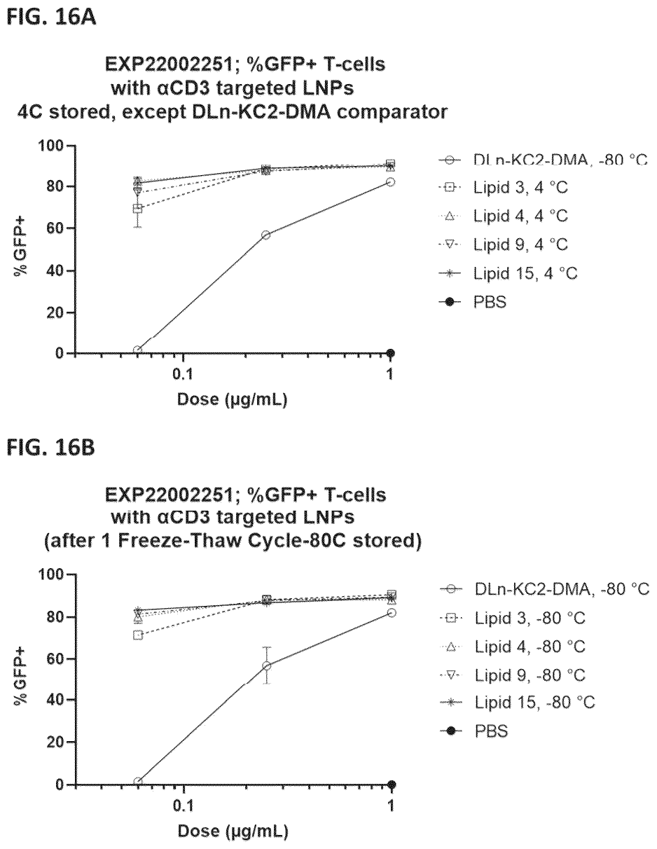

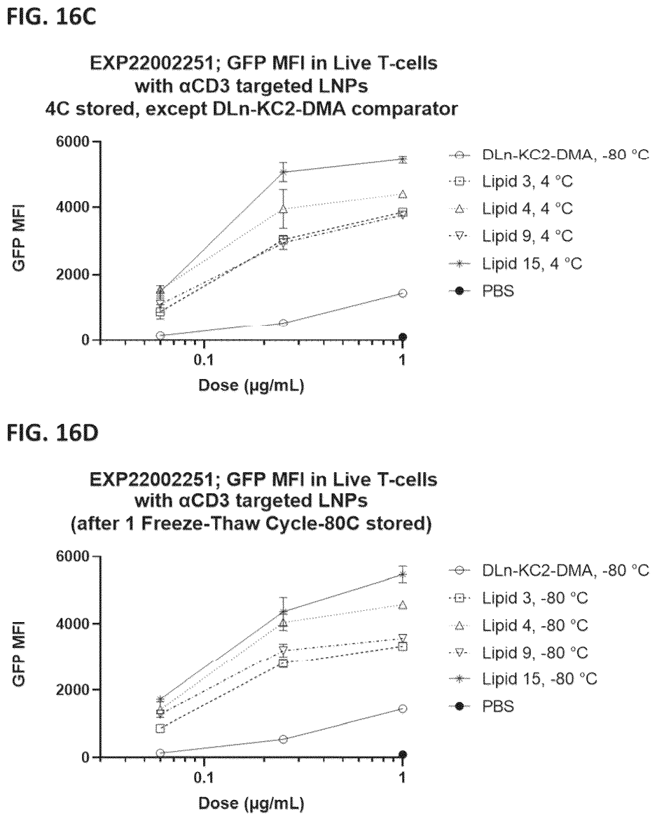

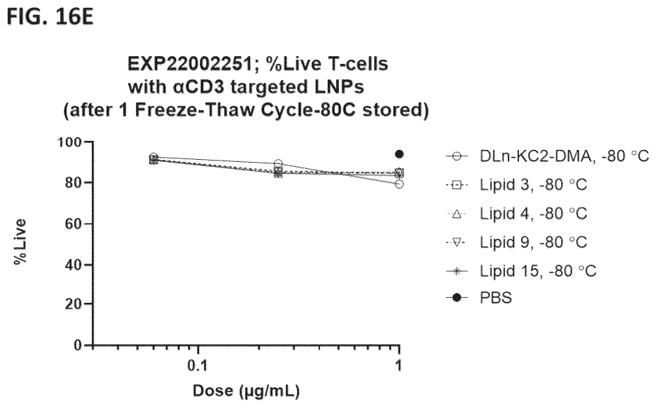

This example compares the GFP protein expression resulting from LNP's derived from Lipids 3, 4, 9, 15 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An &CD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, performance improvements (both in terms of fraction of GFP+ T-cells as reflected by % GFP+ cells and the copies of GFP protein produced on a per cell basis as reflected by GFP MFI values) were observed between lipid 3 versus lipid 4 and between lipid 9 versus lipid 15 indicating that the oleoyl tail groups (in Lipids 4 and 15) result in improved performance over the corresponding linoleoyl tail groups (in Lipids 3 and 9). Thus, gains in lipid oxidative stability via lower lipid tail unsaturation (with singly unsaturated oleic acid) without loss of LNP efficiency typically observed with the lower lipid membrane fluidity. Furthermore, in contrast to Lipid 4 where poorer LNP size distribution properties were observed (relative to Lipid 3), Lipid 15 LNPs exhibited size distribution properties similar to those observed with Lipid 9 LNPs indicating that the N-acyl substituent plays a more significant role in determining the LNPs size distribution properties compared to the role of the lipid tail groups. The performance levels and relative order of LNP efficiency was well preserved in all formulation tested after 1 Freeze Thaw cycle (Post −80° C. storage) as illustrated by comparison of % GFP+ cells and GFP MFI values before and after −80° C. storage (FIG. 16A verses FIG. 16B and FIG. 16C versus FIG. 16D). Lipids 3, 4, 9, and 15 LNPs were well tolerated by primary human T-cells (FIG. 16E).

### Example 29. In Vitro Protein Expression (GFP) in Primary Human T-Cells of Lipids 3, 4, 9, 15 aCD8 (TRX-2) Targeted LNPs and the Corresponding Non-Targeted Parent LNPs (Both Stored at 4° C.)

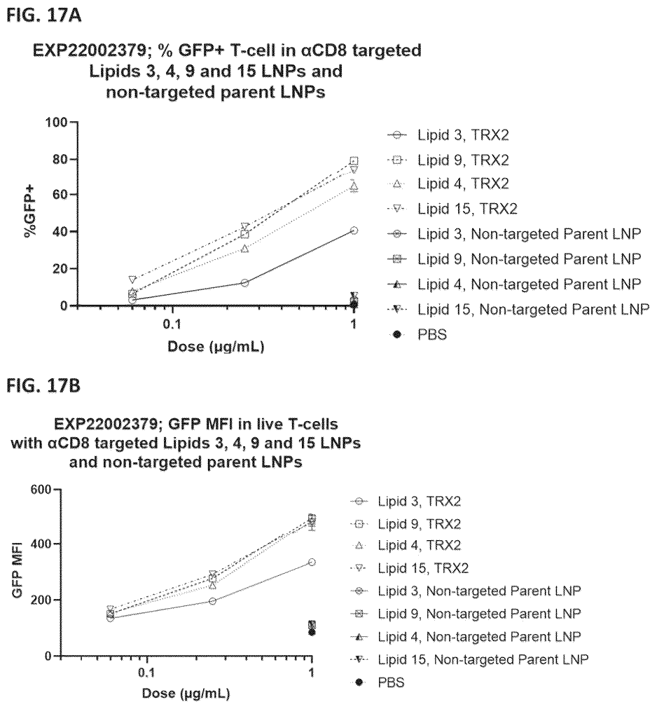

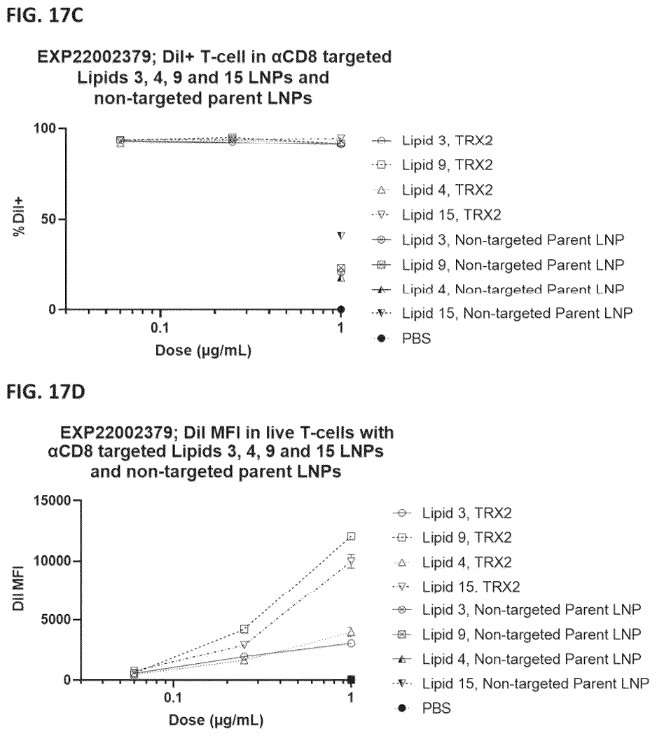

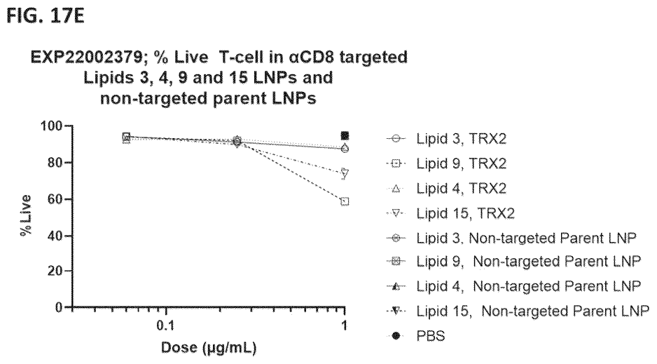

This example compares the GFP protein expression resulting from LNP's derived from Lipids 3, 4, 9, 15 and the corresponding non-targeted parent LNPs. Nanoparticles bearing GFP encoding mRNA (and optionally a fluorescent dye label (DiI-C18-5DS)) were produced using the microfluidic mixing and buffer exchange processes described in Example 2. An &CD8 Fab-conjugate, TRX2, were incorporated into the parent LNPs to obtain the final Antibody targeted LNP formulation using the process described in Example 5. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8 T-cell population. As seen in FIG. 17A and FIG. 17B, Lipids 9 and 15 resulted in similar levels of GFP protein expression the CD8 T-cell population (both in terms of fraction of GFP+ T-cells as reflected by % GFP+ cells and the copies of GFP protein produced on a per cell basis as reflected by GFP MFI values). FIG. 17C and FIG. 17D, show the % DiI+ (dye) T-cells and the Dil MFI reflective of the relative levels of Dil dye labeled LNPs taken up by the CD8 T-cell population. Notably, similar % DiI+ T-cells were observed in all targeted formulations while buffer control-like dye levels (DiI MFI values, FIG. 17 D) were seen in parent (non-targeted) formulations confirming the role of the TRX2 targeting Fab in association and uptake into CD8 T-cells. As expected, no GFP protein expression was observed in the non-targeted parent LNP formulations indicating lipid chemistry did not play a role this TRX2 mediated cellular uptake mechanism. Lipids 3, 4, 9, and 15 αCD8 (TRX2) targeted LNPs were well tolerated by primary human T-cells (FIG. 17E) with cell viability trending a measurable lower in Lipid 9 and Lipid 15 formulations possibly due to the higher levels of GFP protein expression observed with these lipids (as indicated by the higher GFP MFI values seen FIG. 17B).

### Example 30. In Vitro Protein Expression (GFP) in Primary Human T-Cells of Lipids 3, 4, 9, 15 aCD8 (T8) Targeted LNPs and the Corresponding Non-Targeted Parent LNPs (Both Stored at 4° C.)

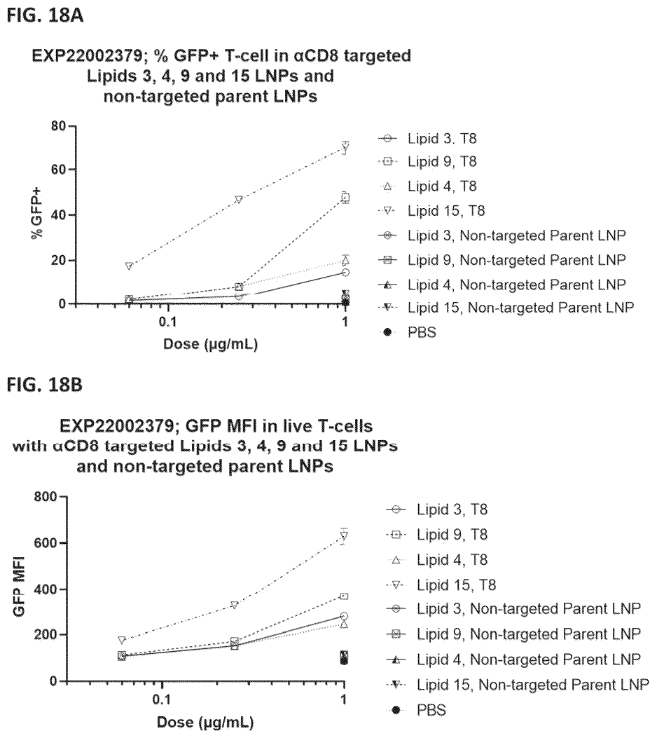

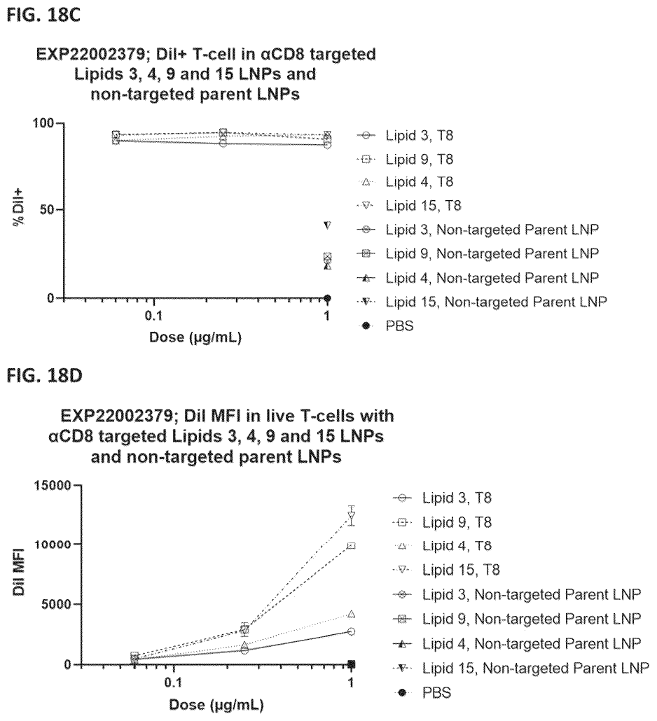

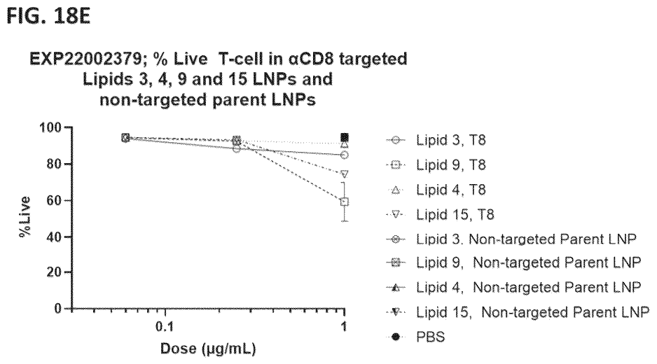

This example compares the GFP protein expression resulting from LNP's derived from Lipids 3, 4, 9, 15 and the corresponding non-targeted parent LNPs (both after one Freeze-Thaw cycle). Nanoparticles bearing GFP encoding mRNA (and optionally a fluorescent dye label (Dil-C18-5DS)) were produced using the microfluidic mixing and buffer exchange processes described in Example 2. An αCD8 Fab-conjugate, T8, was incorporated into the parent LNPs to obtain the final Antibody targeted LNP formulation using the process described in Example 5. Particles thus produced were tested in vitro in primary human CD8 T cells to assess reported gene expression. Additionally, parent LNPs (without any targeting Fab conjugate incorporated into the LNP corona) were tested to check any non-specific uptake in the CD8 T-cell population. As seen in FIG. 18A and FIG. 18B, with this T8 &CD8 targeting strategy, Lipid 15 LNPs out-performed Lipid 9 LNPs with 2-3 fold higher reporter gene expression observed (both in terms of fraction of GFP+ T-cells as reflected by % GFP+ cells and the copies of GFP protein produced on a per cell basis as reflected by GFP MFI values). FIG. 18C and FIG. 18D, show the % DiI+ (dye) T-cells and the Dil MFI reflective of the relative levels Dil dye labelled LNPs taken up by the CD8 T-cell population. Notably, similar % DiI+ T-cells were observed in all targeted formulations while buffer control-like dye levels (DiI MFI values, FIG. 18 D) were seen in parent (non-targeted) formulations confirming the role of the T8 targeting Fab in association and uptake into CD8 T-cells. As expected, no GFP protein expression was observed with the non-targeted parent LNP formulations indicating lipid chemistry did not play a role this T8 mediated cellular uptake mechanism. Lipids 3, 4, 9, and 15 αCD8 (T8) targeted LNPs were well tolerated by primary human T-cells (FIG. 18E) with cell viability trending a measurable lower in Lipid 9 and Lipid 15 formulations possibly due to the higher levels of GFP protein expression observed with these lipids (as indicated by the higher GFP MFI values seen FIG. 18B).

### Example 31. In Vitro Protein Expression (GFP) in Primary Human T-Cells of Lipids 2, 3, 31 and 32 and Comparator Lipid (DLin-KC2-DMA) aCD3 (Hsp34) Targeted LNPs Stored at 4° C.

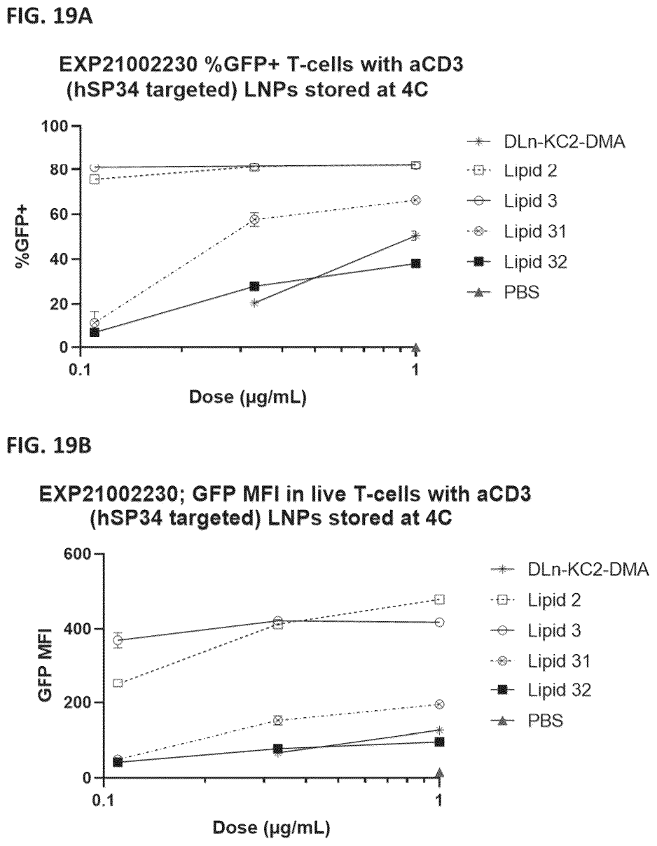

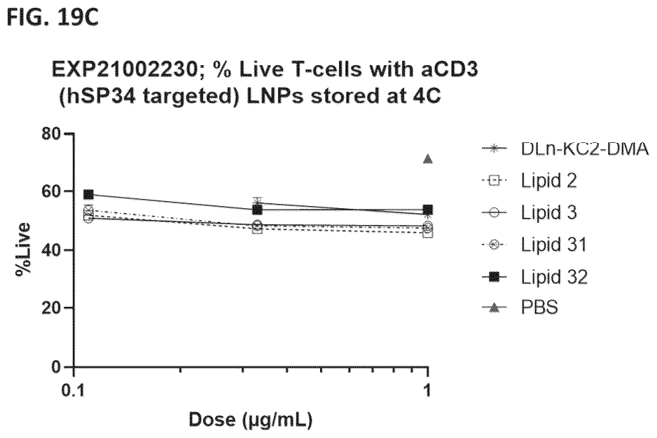

This example compares the GFP protein expression resulting from LNP's derived from Lipids 2, 3, 31 and 32 to LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. At all dose levels, Lipid 2 and 3 out-performed Lipid 31, 32 and DLin-KC2-DMA LNPs both in terms of fraction of GFP+ T-cells (reflected by % GFP+ cells) and the copies of GFP protein produced on a per cell basis (reflected by GFP MFI values) (FIG. 19A and FIG. 19B). This is consistent with higher apparent pKa of Lipid 31 and Lipid 32 LNPs relative to Lipid 2 and 3 LNPs and less pronounced change in LNP charge state under acidic endosomal pH conditions (as described in Example 13) and consequently lower levels of cytosolic access expected with Lipids 31 and 32. Lipids 2, 3, 31 and 32 LNPs were well tolerated by primary human T-cells (FIG. 19C).

### Example 32. In Vitro Protein Expression (GFP) in Primary Human T-Cells of Lipids 3, 33 and 34 and Comparator Lipid (DLin-KC2-DMA) aCD3 Targeted LNPs Stored at 4° C. and after 1 Freeze-Thaw Cycle (Post −80° C. Storage) and Non-Binding (Mutated OKT8) Antibody Targeted LNPs

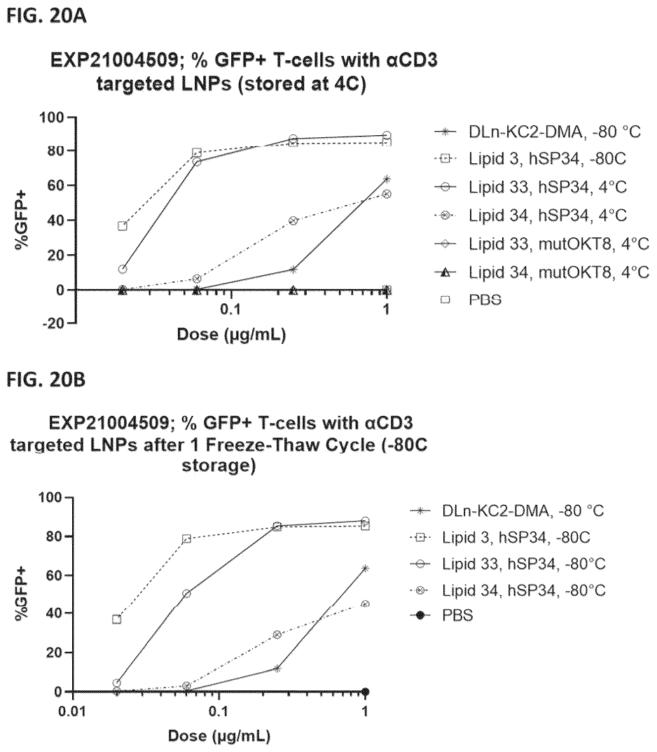

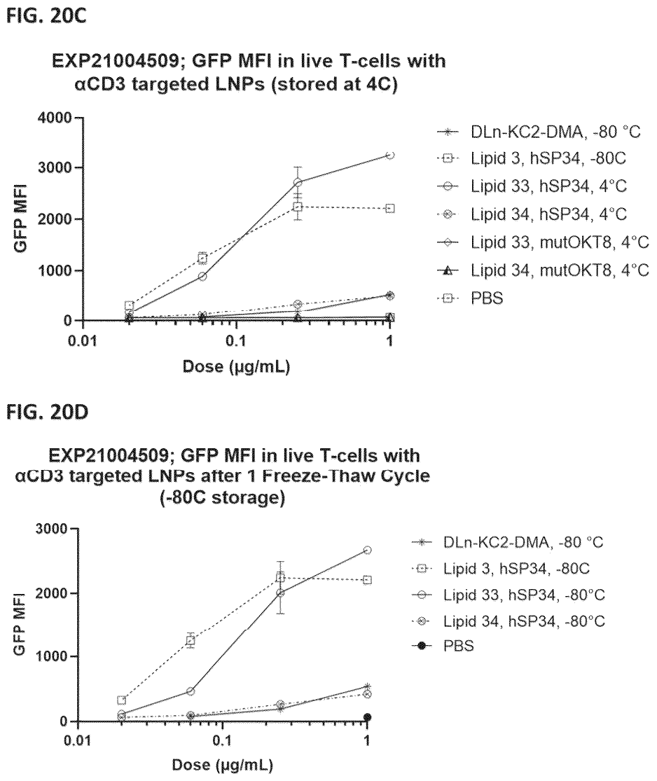

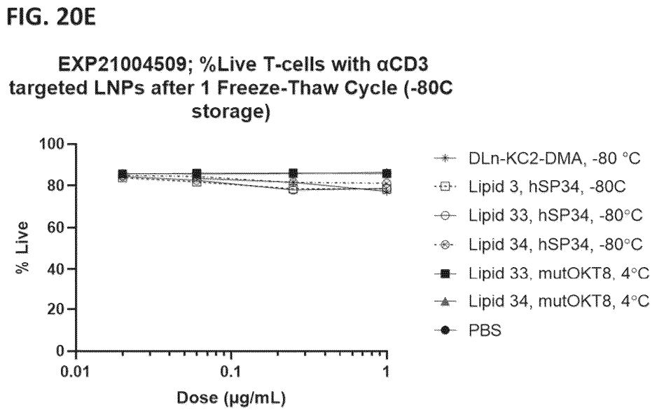

This example compares the GFP protein expression resulting from LNP's derived from Lipids 3, 31, 32 and LNPs made using comparator lipid DLin-KC2-DMA. Nanoparticles bearing GFP encoding mRNA (and optionally a fluorescent dye label (DiI-C18-5DS)) were produced using the microfluidic mixing and buffer exchange processes described in Example 2. An αCD3 Fab-conjugate was incorporated into the parent LNPs to obtain the final Antibody targeted LNP formulation using the process described in Example 5. Additionally, mock targeted LNPs were produced by incorporation of a non-binding mutated Antibody Fab (mut-OKT8) conjugate into the parent LNPs using the Antibody conjugate insertion method described in Example 4. Particles thus produced were tested in vitro in primary human CD3+ T cells to assess reported gene expression. At all dose levels, Lipid 3 and 33 out-performed Lipid 34 and DLin-KC2-DMA LNPs both in terms of fraction of GFP+ T-cells (reflected by % GFP+ cells) and the copies of GFP protein produced on a per cell basis (reflected by GFP MFI values). Furthermore, mut-OKT8 functional Lipid 33 and Lipid 34 LNPs did not result in any protein expression confirming the role of the αCD3 Antibody (hSP34) in the cellular uptake mechanism observed with these lipid formulations. The performance levels and relative order of LNP efficiency was well preserved in all formulations tested after 1 Freeze Thaw cycle (Post −80° C. storage) as illustrated by comparison of % GFP+ cells and GFP MFI values before and after −80° C. storage (FIG. 20A verses FIG. 20B and FIG. 20C versus FIG. 20D). Lipids 3, 33 and 34 LNPs were well tolerated by primary human T-cells (FIG. 20E).

### Example 33. In Vitro Car Protein Expression (M1 Tagged Extracellular Domain) in Primary Human T-Cells of Lipids 3, 4, 9 and 34, aCD3 Targeted aCD20 Car-RNA LNPs Stored at 4° C. and after 1 Freeze-Thaw Cycle (Post −80° C. Storage)

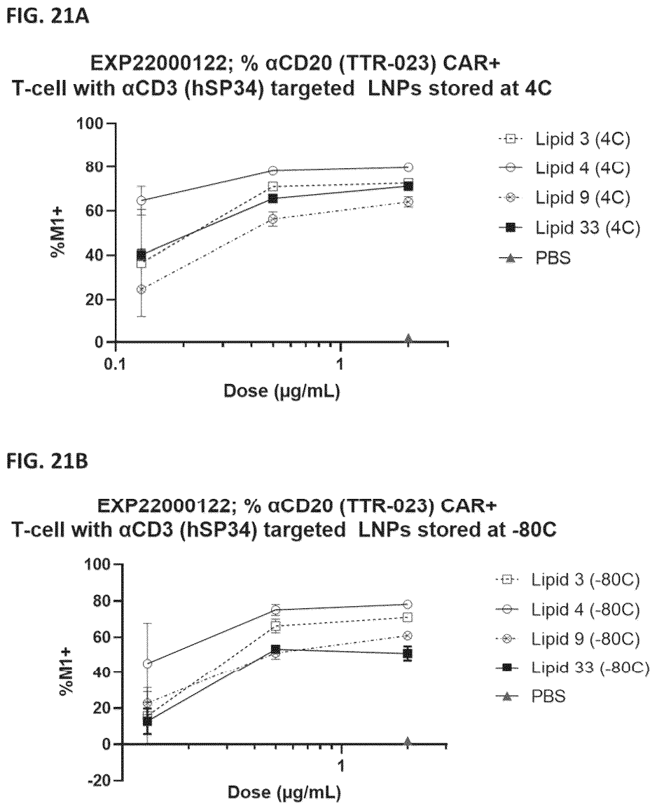

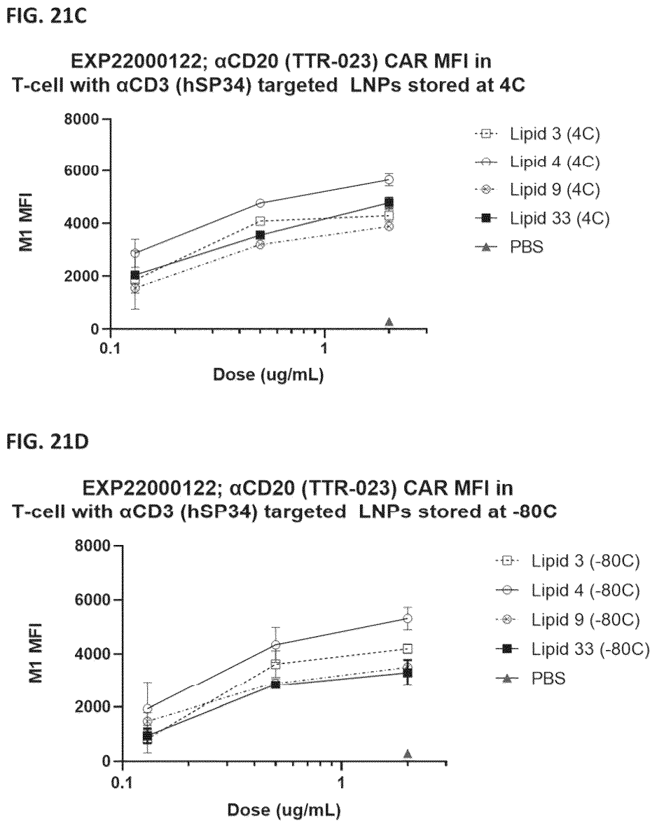

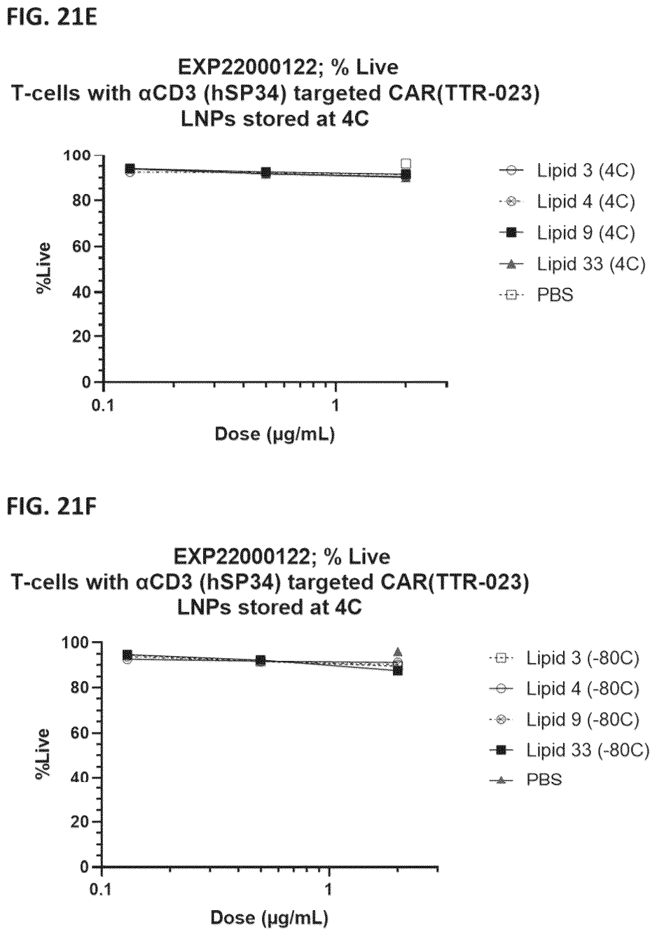

This example compares the &CD20 CAR (TTR-023) protein expression resulting from LNP's derived from Lipids 3, 4, 9 and 33. Nanoparticles bearing an &CD20 CAR encoding mRNA (TTR-023) were produced using the microfluidic mixing and buffer exchange processes described in Example 2. An αCD3 Fab-conjugate (hSP34) was incorporated into the parent LNPs to obtain the final Antibody targeted LNP formulation using the process described in Example 5. Particles thus produced were tested in vitro in primary human CD3+ T cells to assess CAR expression via detection of an M1 tag on the extracellular domain of the TTR-023 transmembrane CAR protein. At all dose levels tested, higher CAR levels were detected with lipids 3 and 4 relative to lipids 9 and 33 with Lipid 4 performing the best in this &CD3 targeting pathway. The relative levels of CAR expression are consistent with oleoyl tail group of Lipid 4 resulting in improved performance over the corresponding linoleoyl tail group of Lipids 3 and 9 seen with reported (GFP) gene expression in Examples 28, 29 and 30. Furthermore, this illustrates that this relative order of lipid efficiency is preserved between a reporter gene (GFP) and a therapeutic cargo (TTR-023 CAR protein) in this αCD3 mediated targeting and cellular uptake mechanism. The performance levels and relative order of LNP efficiency was well preserved in Lipid 3, 4 and 9 LNPs after 1 Freeze Thaw cycle (Post −80° C. storage). In contrast, a drop in CAR expression was detected at all doses of Lipid 33 LNP as illustrated by comparison of % M1+ cells and M1 MFI values before (4° C. stored) and after −80° C. storage (FIG. 21A verses FIG. 21B and FIG. 21C versus FIG. 21D). Lipids 3, 4, 9, and 33 LNPs were well tolerated by primary human T-cells (FIG. 21E and FIG. 21F).

### Example 34. In Vitro Car Protein Expression (M1 Tagged Extracellular Domain) in Primary Human T-Cells of Lipids 3, 4, 9 and 34, aCD8 (T8) Targeted aCD20 Car-RNA LNPs Stored at 4° C.

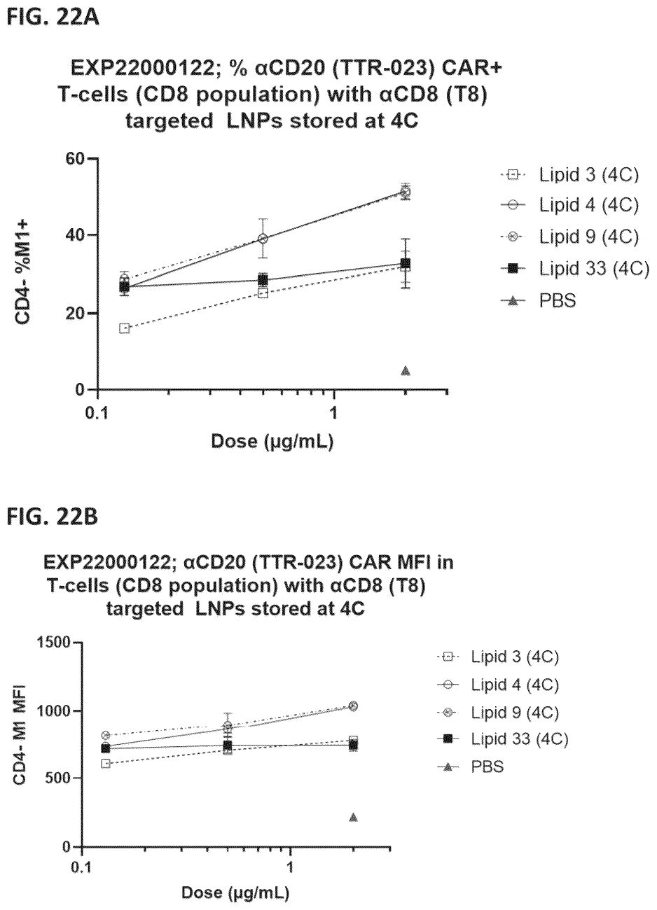

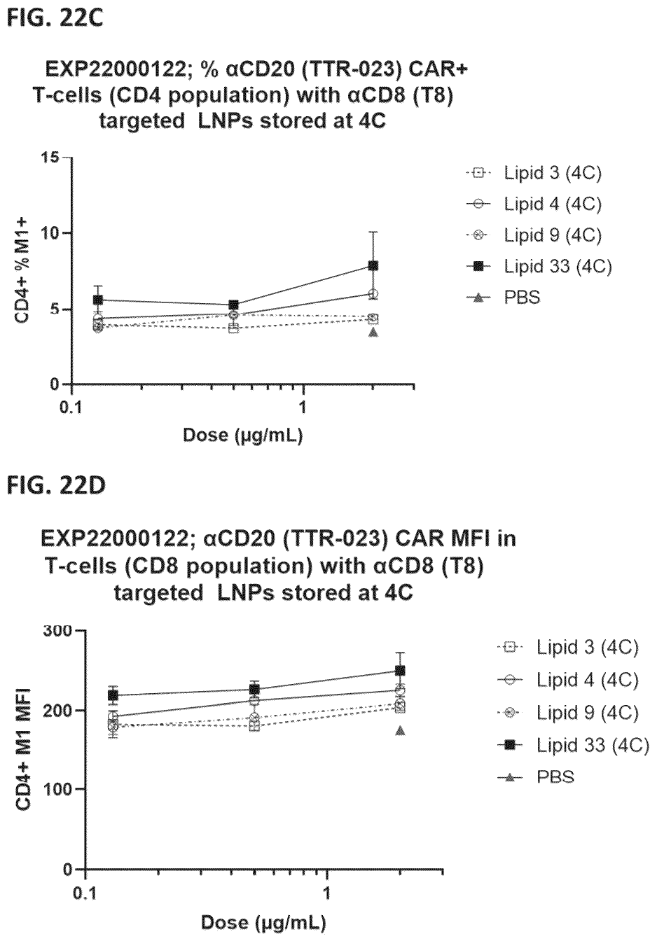

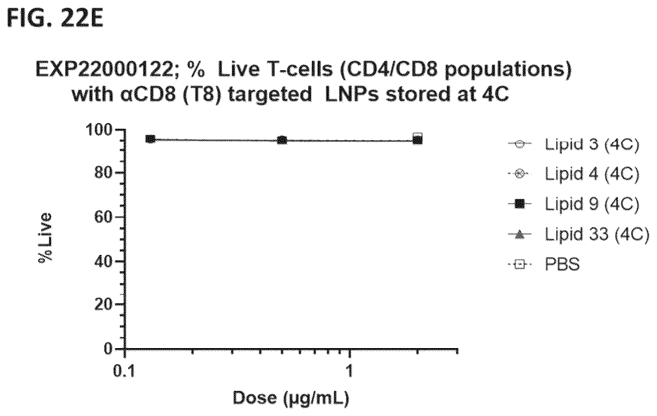

This example compares the αCD20 CAR (TTR-023) protein expression resulting from LNP's derived from Lipids 3, 4, 9 and 33. Nanoparticles bearing an &CD20 CAR encoding mRNA (TTR-023) were produced using the microfluidic mixing and buffer exchange processes described in Example 2. An αCD8 Fab-conjugate (T8) was incorporated into the parent LNPs to obtain the final Antibody targeted LNP formulation using the process described in Example 5. Particles thus produced were tested in vitro in primary human CD3+ T cells to assess CAR expression via detection of an M1 tag on the extracellular domain of the TTR-023 transmembrane CAR protein. Transfected cells were gated as CD4+ (CD4 population) and CD4−(CD8 population) and CAR expression was monitored (as % M1+ and M1 MFI) in the two populations to assess specificity in the CD8 population with the &CD8 (T8) targeting strategy. At all dose levels tested, higher CAR levels in the CD4-population (CD8 cells, as illustrated by the M1% and M1 MFI values in FIG. 22A and FIG. 22B) were detected with lipids 4 and 9 relative to lipids 3 and 33 with Lipids 4 and 9 performing equally well in this CD8 targeting pathway. CAR levels similar to buffer control (PBS) were detected in the CD4+ population (CD4 cells, as illustrated by the M1% and M1 MFI values in FIG. 22C and FIG. 22D) confirming that a T8 antibody enables receptor mediated uptake specifically into CD8 T-cells. Lipids 3, 4, 9, and 33 LNPs were well tolerated by primary human T-cells (FIG. 22E).

### Example 35. In Vitro Car Protein Expression (M1 Tagged Extracellular Domain) in Primary Human T-Cells of Lipids 3, 4, 9 and 34, aCD8 (T8) Targeted aCD20 Car-RNA LNPs after 1 Freeze-Thaw Cycle (Post −80° C. Storage)

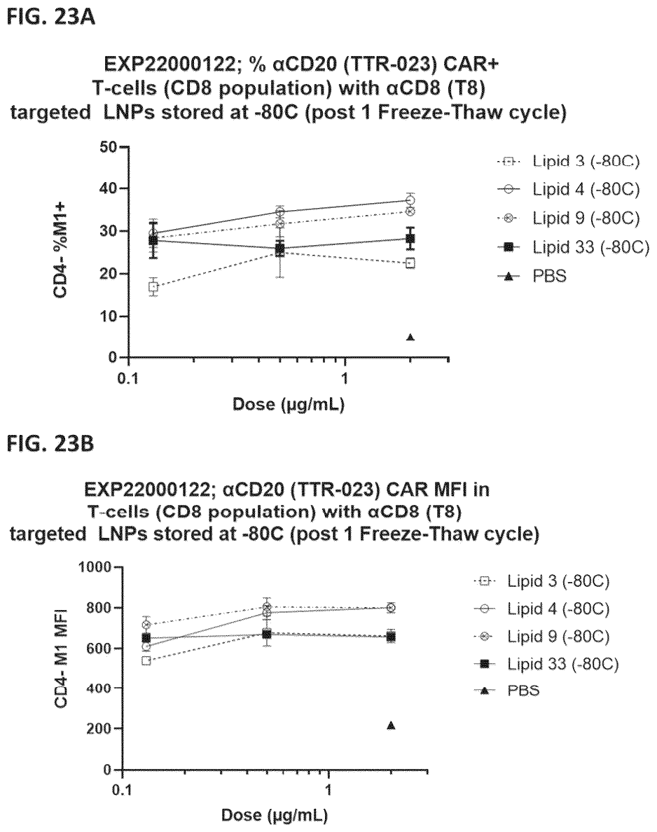

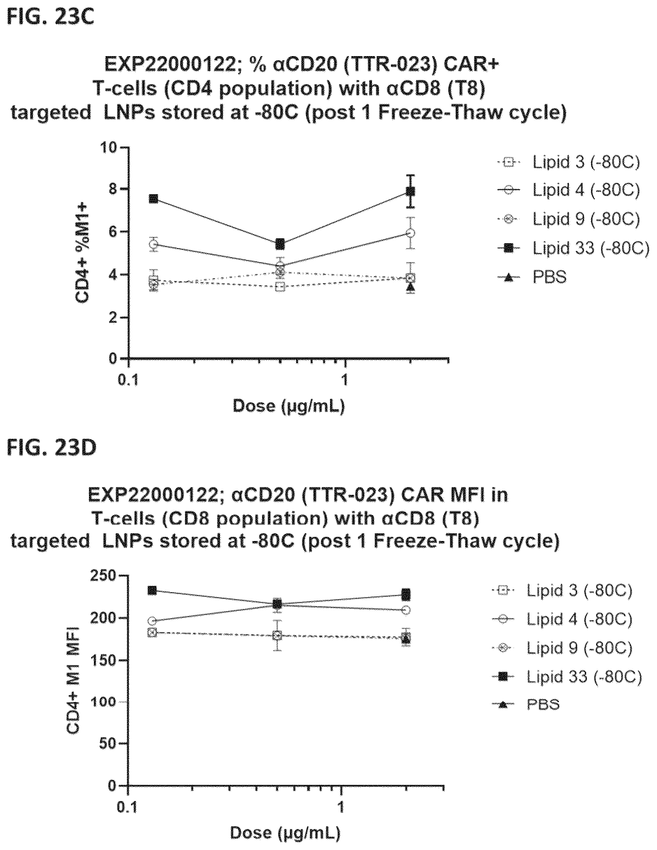

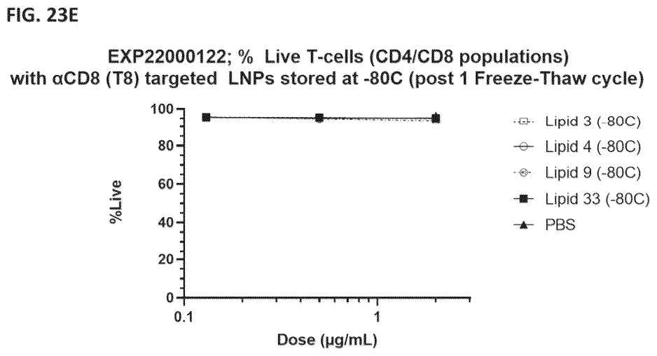

This example compares the αCD20 CAR (TTR-023) protein expression resulting from LNP's derived from Lipids 3, 4, 9 and 33 after 1 Freeze-Thaw cycle (post −80° C. storage). Nanoparticles bearing an αCD20 CAR encoding mRNA (TTR-023) were produced using the microfluidic mixing and buffer exchange processes described in Example 2. An αCD8 Fab-conjugate (T8) was incorporated into the parent LNPs to obtain the final Antibody targeted LNP formulation using the process described in Example 5. Particles thus produced were subjected to 1 Freeze-Thaw cycle and then tested in vitro in primary human CD3+ T cells to assess CAR expression via detection of an M1 tag on the extracellular domain of the TTR-023 transmembrane CAR protein. Transfected T-cells were gated as CD4+ (CD4 population) and CD4−(CD8 population) and CAR expression was monitored (as % M1+ and M1 MFI) in the two populations to assess specificity in the CD8 population with the &CD8 (T8) targeting strategy. At all dose levels tested, higher CAR levels in the CD4-population (CD8 cells, as illustrated by the M1% and M1 MFI values in FIG. 23A and FIG. 23B) were detected with lipids 4 and 9 relative to lipids 3 and 33 while Lipids 4 and 9 performed equally well in this CD8 targeting pathway. CAR levels similar to buffer control (PBS) were detected in the CD4+ population (CD4 cells, as illustrated by the M1% and M1 MFI values in FIG. 23C and FIG. 23D) confirming that a T8 antibody enables receptor mediated uptake specifically into CD8 T-cells. Both levels of CAR expression and specificity to CD8 T-cells were observed with particles that had been subjected to 1 Freeze-Thaw cycle confirming that the integrity and function of the formulations were preserved. Lipids 3, 4, 9, and 33 LNPs were well tolerated by primary human T-cells (FIG. 23E).

### Example 36. GFP Protein Expression and LNP Association (Measured as DiI-Dye Fluorescence) in CD8 and CD4 T-Cell with Lipid 9, 15, DLin-KC3-DMA Lipid aCD3 (Hsp34) Targeted LNPs in Human Whole Blood

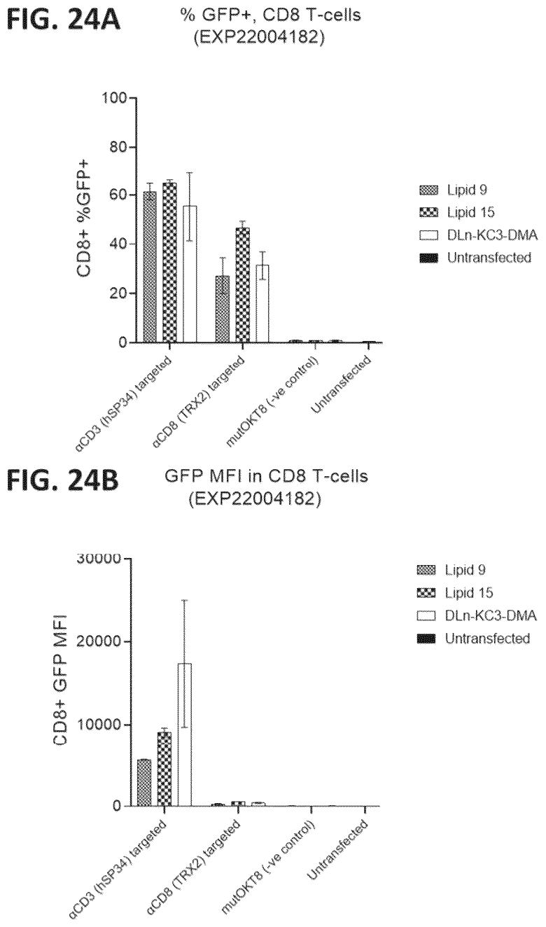

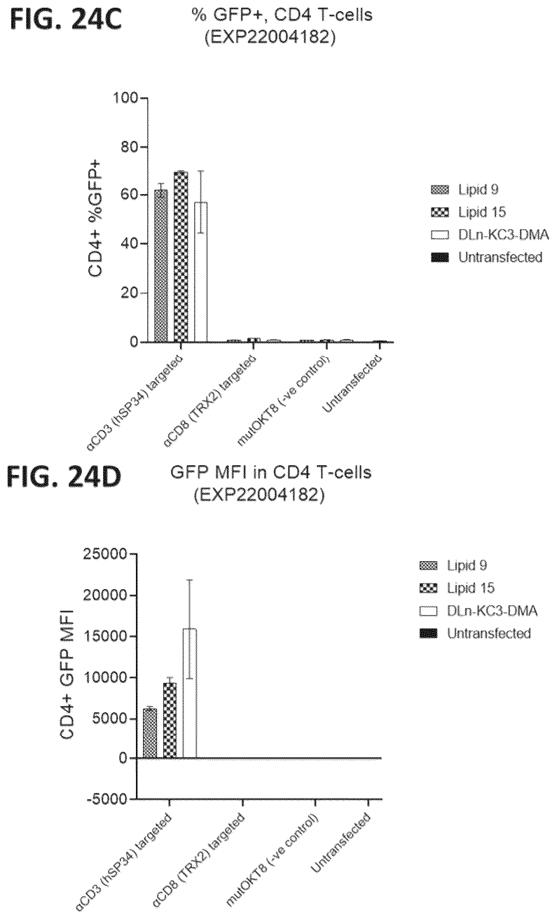

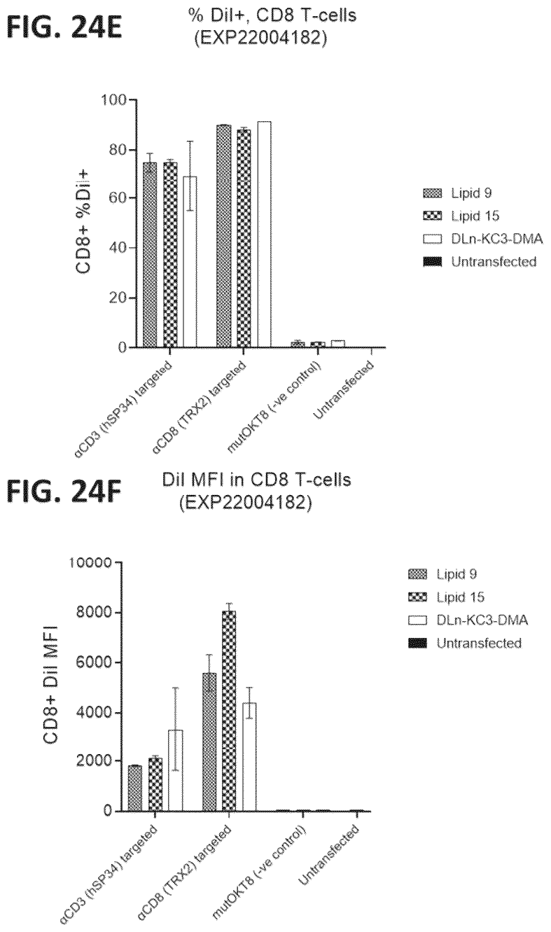

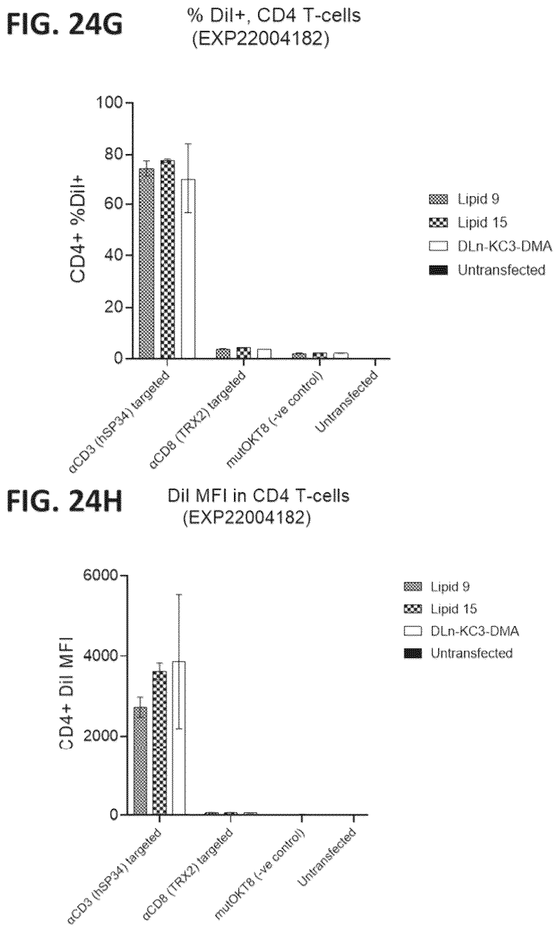

Lipid 9, 15 and DLin-KC3-DMAαCD3 and αCD8 targeted (and a non-binding Antibody, mutOKT8, as a negative control) GFP mRNA LNPs were dosed to human venous whole blood, incubated for 24 hours and analyzed using the protocol described in Example 21 for whole blood transfections. As seen in FIG. 24A, FIG. 24B, FIG. 24C, and FIG. 24D, αCD3 (hSP34) targeted LNPs resulted in GFP expression in both CD4 and CD8 T-cells while αCD8 (TRX2) targeted LNPs resulted in GFP expression selective in CD8 T-cells only as expected. Furthermore, non-binding (mutOKT8) control LNP resulted in no GFP expression in either cell type. Lipid 9 and 15 αCD3 (hSP34) targeted LNPs exhibited similar levels of GFP expression indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of CD8 T-cells (expressed as % GFP+ cells) as well as more copies of GFP protein on a per cell basis (expressed as GFP MFI). As seen in FIG. 24E, FIG. 24F, FIG. 24G, and FIG. 24H, αCD3 (hSP34) targeted LNPs resulted in binding to both CD4 and CD8 T-cells while αCD8 (TRX2) targeted LNPs bound selectively to CD8 T-cells only (measure as DiI dye % and MFI in the two cell populations). Furthermore, non-binding (mutOKT8) control LNP resulted in no significant binding to either T-cell type as expected. Lipid 9 and 15 LNPs bound to similar fractions of CD4 and CD8 T-cells as indicated by similar % DiI+ cells in both populations. However slightly higher binding was observed with Lipid 15 LNPs in the CD8 binding and uptake pathway relative to Lipid 9 LNPs as indicated by brighter dye fluorescence intensity (DiI MFI values) on a per cell basis.

### Example 37. GFP Protein Expression in NK-Cells, Granulocytes, and B-Cells, with Lipid 9, 15, DLin-KC3-DMA Lipid αCD3 (Hsp34) Targeted LNPs in Human Whole Blood

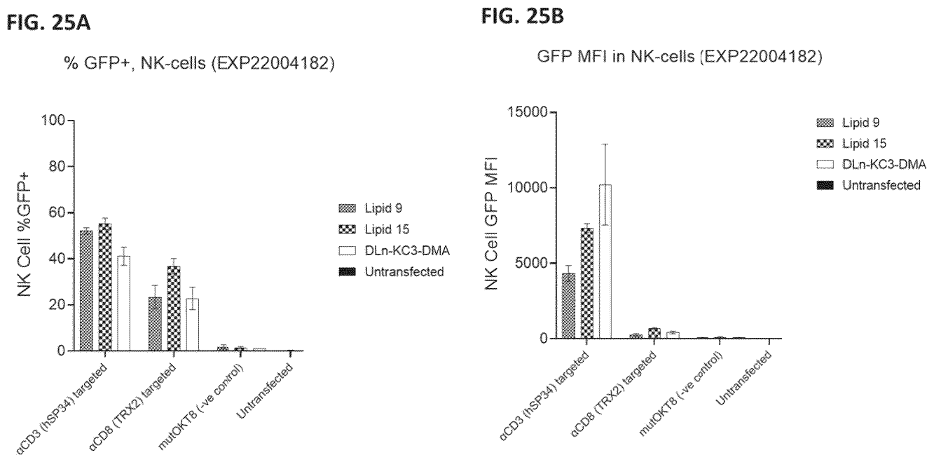

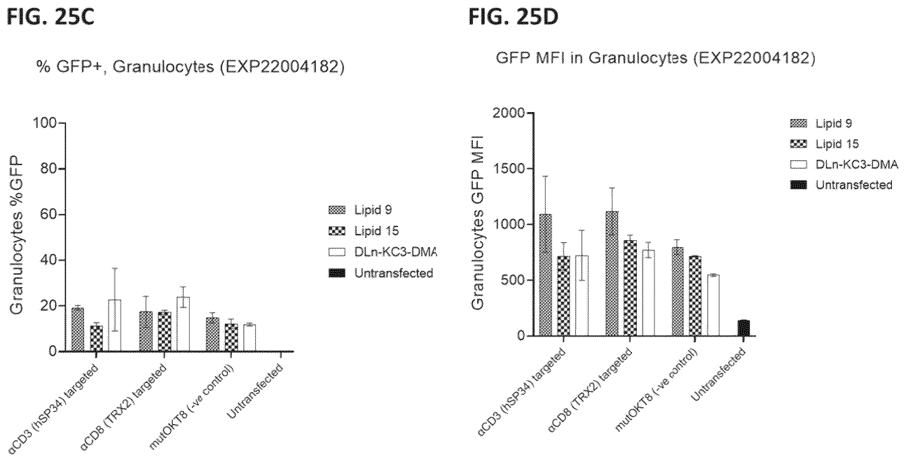

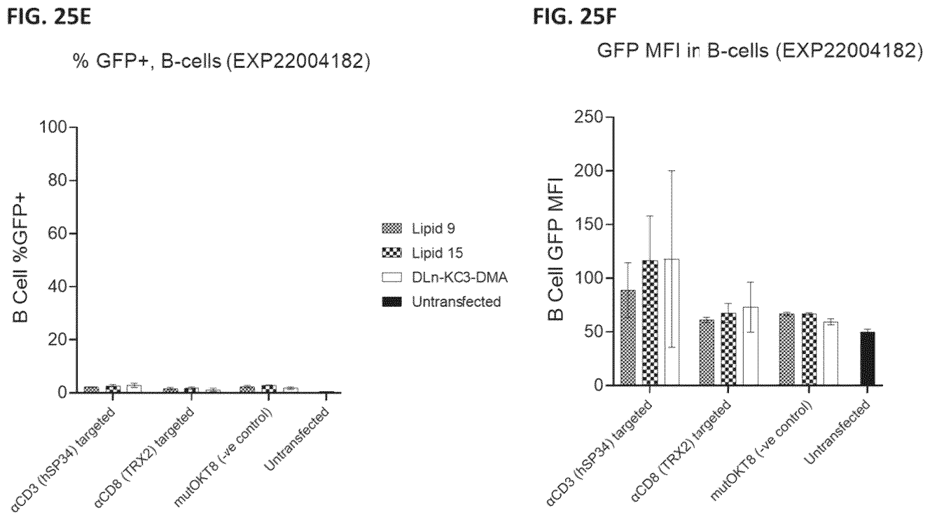

Lipid 9, 15 and DLin-KC3-DMAαCD3 and αCD8 targeted (and a non-binding Antibody, mutOKT8, as a negative control) GFP mRNA LNP transfected whole blood samples (of Example 38) were also analyzed for GFP expression in NK-cells, Granulocytes and B-cells using the protocol described in Example 21 for whole blood transfections. As seen in FIG. 25A and FIG. 25B, both αCD3 (hSP34) targeted LNPs and αCD8 (TRX2) targeted LNPs resulted in GFP expression in NK-cells as expected. Furthermore, no GFP expression was observed in NK-cells with the non-binding (mutOKT8) control LNP. Lipid 9 and 15 αCD3 (hSP34) targeted LNPs exhibited similar levels of GFP expression in NK-cells indicating that the two lipids are equally efficient in this cellular uptake pathway. However, in the CD8 binding and uptake pathway, αCD8 (TRX2) targeted Lipid 15 LNPs outperformed the corresponding Lipid 9 LNPs transfecting both a larger fraction of NK-cells (expressed as % GFP+ cells) as well as more copies of GFP protein on a per cell basis (expressed as GFP MFI). As seen in FIG. 25C, FIG. 25D, FIG. 25E, and FIG. 25F, neither targeting modalities resulted in any significant GFP expression in Granulocytes and B-cells.

### Example 38. LNP Association (Measured as DiI-Dye Fluorescence) in NK-Cells, Granulocytes, and B-Cells with Lipid 9, 15, DLin-KC3-DMA Lipid αCD3 (Hsp34) Targeted LNPs in Human Whole Blood

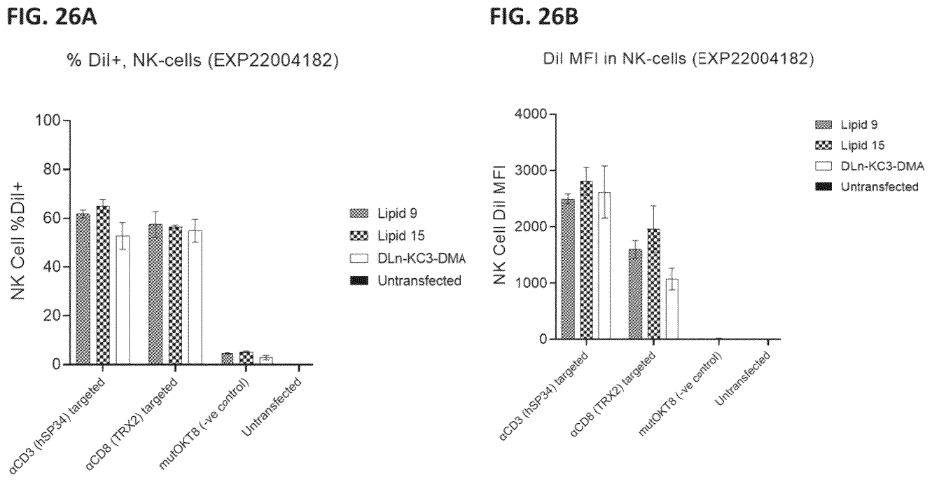

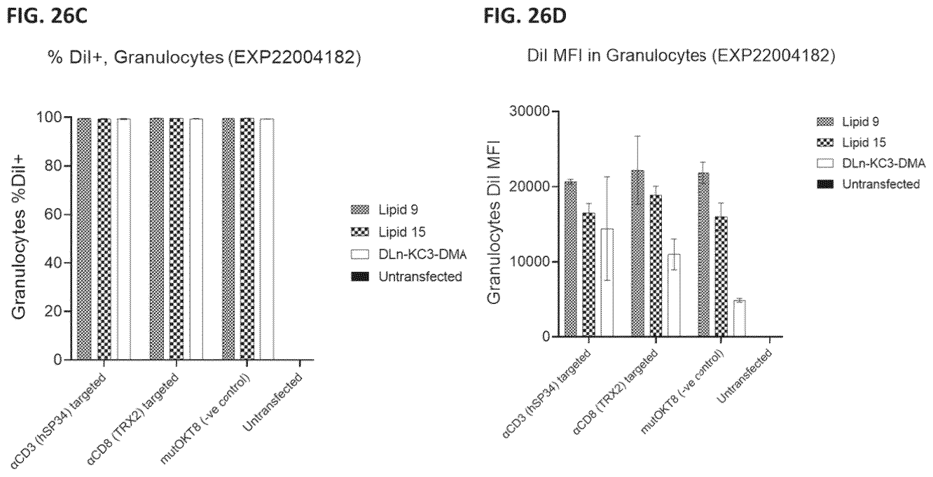

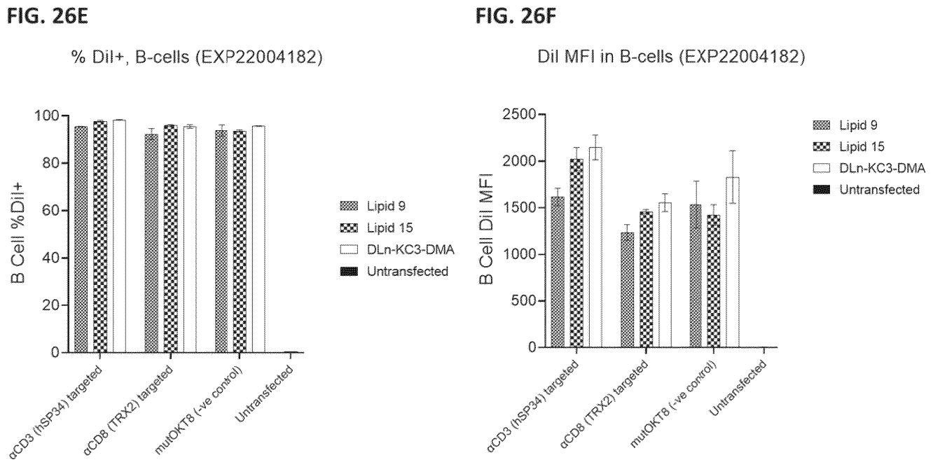

Lipid 9, 15 and DLin-KC3-DMAαCD3 and αCD8 targeted (and a non-binding Antibody, mutOKT8, as a negative control) GFP mRNA LNP transfected whole blood samples (of Example 36) were also analyzed for binding to NK-cells, Granulocytes and B-cells using the protocol described in Example 23 for whole blood transfections. As seen in FIG. 26A and FIG. 26B, both αCD3 (hSP34) targeted LNPs and αCD8 (TRX2) targeted LNPs bound to NK-cells as expected. Furthermore, the non-binding (mutOKT8) control LNP did not result in any significant bind to NK-cells as expected. As seen in FIG. 26C, FIG. 26D, FIG. 26E, and FIG. 26F, both targeting modalities resulted in non-specific binding to Granulocytes and B-cells however as reported in Example 39 above, no GFP expression was observed indicating that RNA was not delivered to the cytosol of either cell type.

### Example 39. In Vitro CAR (TTR-023) and mCherry Expression in Primary Human T-Cells Transfected with αCD8 (TRX2) Targeted Lipid 9 and DLin-KC3-DMA LNPs

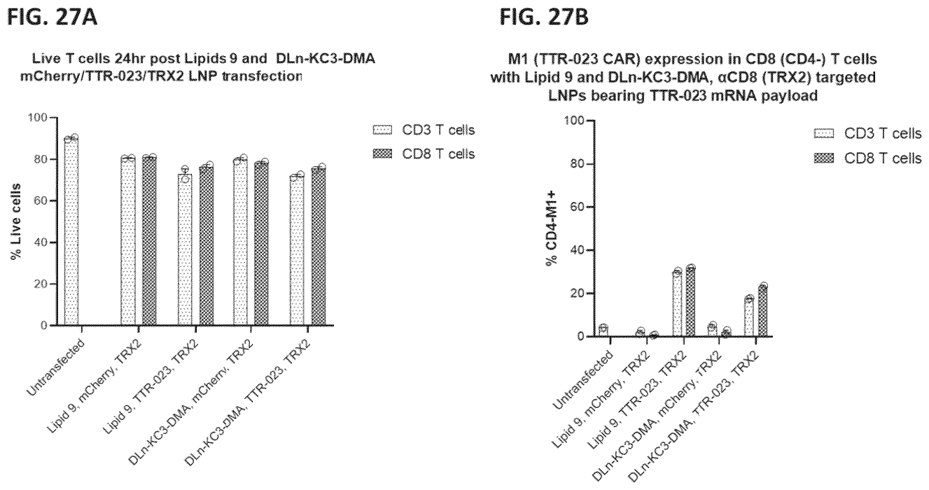

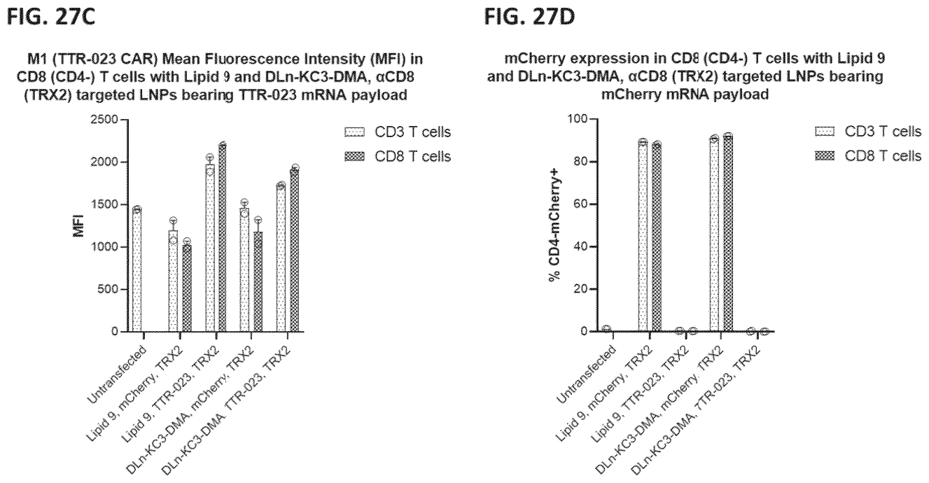

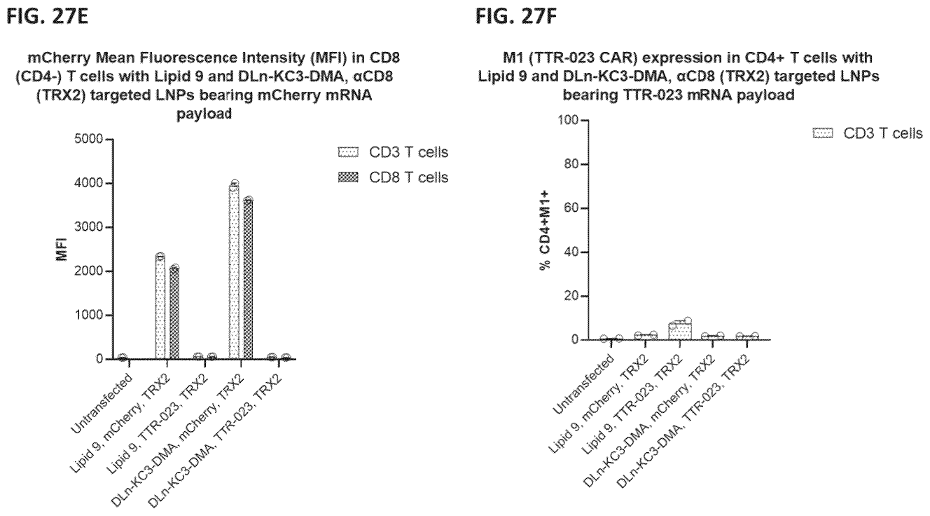

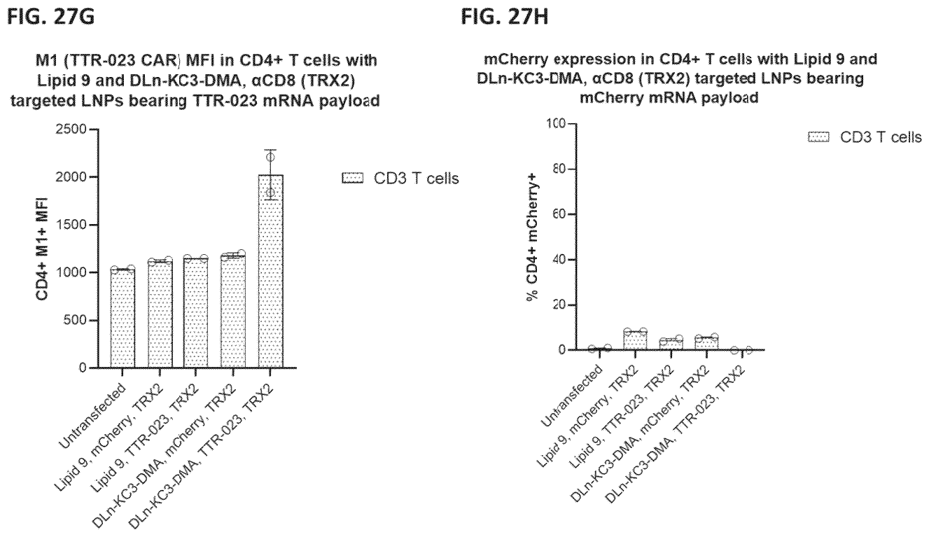

This example compares reporter protein (mCherry) and αCD20 CAR (TTR-023) protein expression resulting from LNP's derived from Lipid 9 to comparator Lipid DLin-KC3-DMA. Nanoparticles bearing mCherry mRNA or an αCD20 CAR encoding mRNA (TTR-023) were produced using the microfluidic mixing and buffer exchange processes described in Example 2. An αCD8 Fab-conjugate (TRX2) was incorporated into the parent LNPs to obtain the final Antibody targeted LNP formulation using the process described in Example 5. Particles thus produced were tested in vitro in primary human CD3+ T cells to assess protein (mCherry or CAR) expression via mCherry fluorescence or detection of an M1 tag on the extracellular domain of the TTR-023 transmembrane CAR protein, respectively. Transfected cells were gated as CD4+ (CD4 population) and CD4−(CD8 population) and levels of protein expression monitored (as % M1+ and M1 MFI for CAR expression or reporter protein fluorescence for mCherry expression) in the two populations were used to assess specificity in the CD8 population with this αCD8 (TRX2) targeting strategy. Protein expression was selectively observed in the CD4-population (CD8 cells, as illustrated by the M1% and M1 MFI values in FIG. 27B and FIG. 27C versus F and G or mCherry % and mCherry MFI values in FIG. 27D and FIG. 27E versus FIG. 27G and FIG. 27H) with both lipid 9 and comparator lipid DLin-KC3-DMA formulations confirming that the TRX2 antibody enables receptor mediated uptake specifically into CD8 T-cells. Lipid 9 LNPs outperformed DLin-KC3-DMA LNPs in CAR expression levels while DLin-KC3-DMA LNPs outperformed Lipid 9 LNPs in mCherry expression levels suggesting that different optimal lipid compositions may be required for expression of intracellular proteins versus membrane bound proteins. Both TRX2 targeted lipid formulations with either CAR or mCherry payloads were well tolerated by primary human T-cells at the 1 μg/mL per 500,000 T-cells dose level (FIG. 27A).

### Example 40. In Vitro Car-T Cell Function by Raji (B-Cell) Co-Culture with aCD20 CAR (TTR-023) Expressing T-Cells Derived by Transfection of Primary Human T-Cells (of Example 27) with aCD8 (TRX2) Targeted Lipid 9 and DLin-KC3-DMA LNPs Bearing Car-mRNA or mCherry-mRNA (as Negative Control)

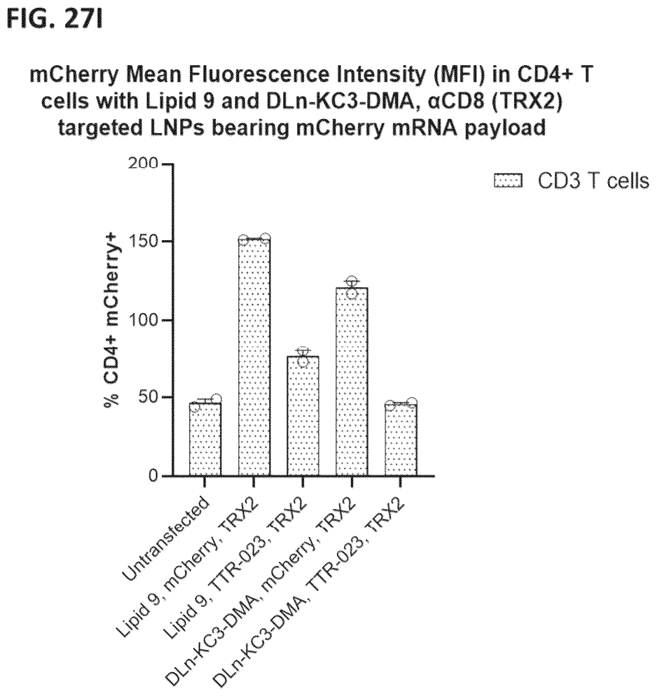

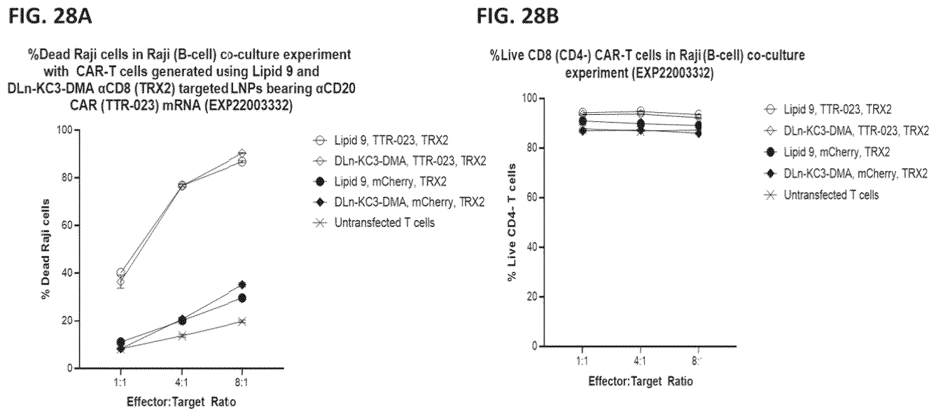

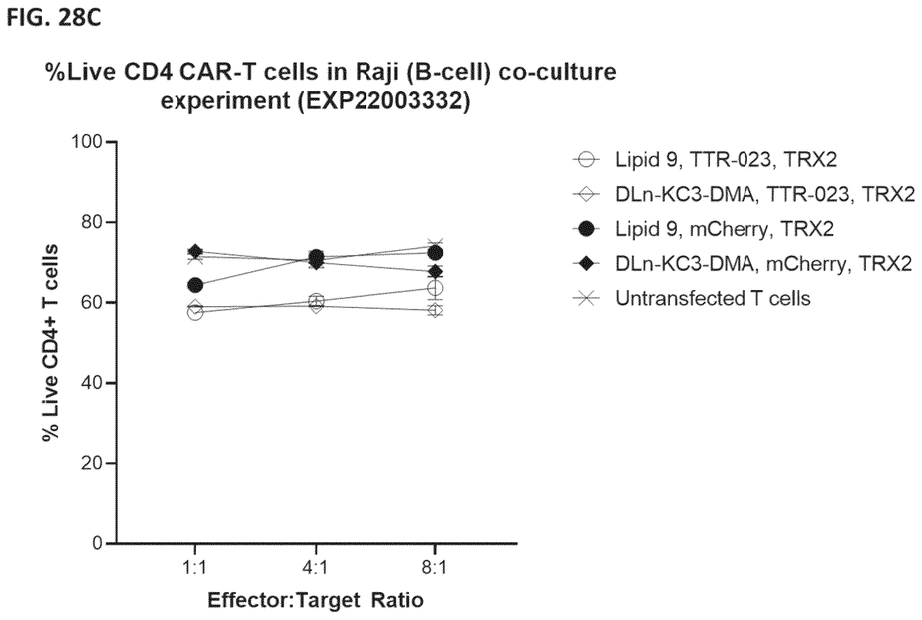

CAR-T cells produced in Example 39 were co-cultured with Raji (B-cells) at Effector: Target (E:T) (T-cell:B-cell) ratios of 1:1, 4:1, and 8:1 for a period of 24 hours and the fraction of Live B-cells and T-cells measured using the protocol described in Example 20. As seen in FIG. 28A, the fraction of dead B-cells increased at higher E:T ratios in a dose-dependent manner. Furthermore, T-cells expressing TTR-023 CAR protein exhibited significantly higher cytotoxicity towards B-cells relative to mCherry transfected T-cells as indicated by a 4× higher % of dead Raji cells at the three E:T ratios tested. This suggests that CAR engagement to target cell CD20 receptor and the downstream target specific granzyme perforin apoptotic pathway plays a major role in the observed T-cell activity while T-cell activation (possibly resulting for CD8 receptor engagement by the TRX2 antibody) over background levels of T-cell cytoxicity towards B-cells is a minor contributor in the overall activity of the CAR-T cells observed here. Both Lipid 9 and DLin-KC3-DMA LNP formulations were equally cytotoxic towards B-cells and both formulations were well tolerated by the CD4 and CD8 T-cells in the co-culture experiment with T-cell viability values remaining either slighty below (CD4 cells) or slightly above (CD4−, CD8 T-cells) the un-transfected controls as seen FIG. 28B and FIG. 28C, respectively.

### Example 41. In Vitro CAR (TTR-023) and mCherry Expression in Primary Human T-Cells Transfected with aCD8 (TRX2) Targeted Lipid 15 and DLin-KC3-DMA LNPs

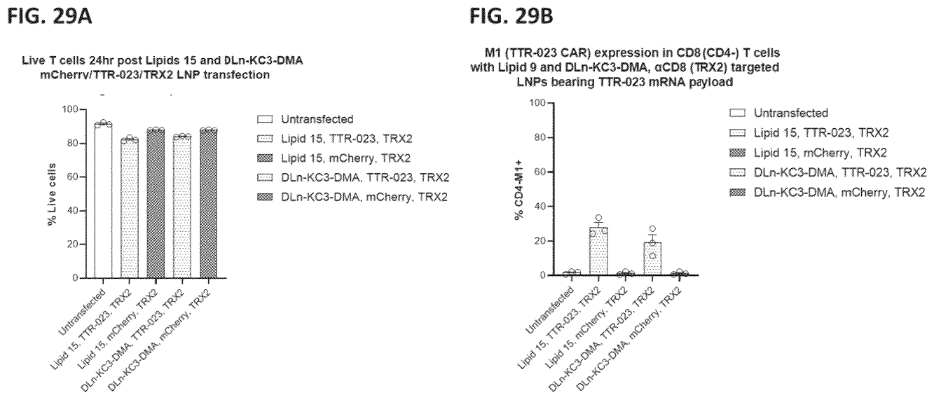

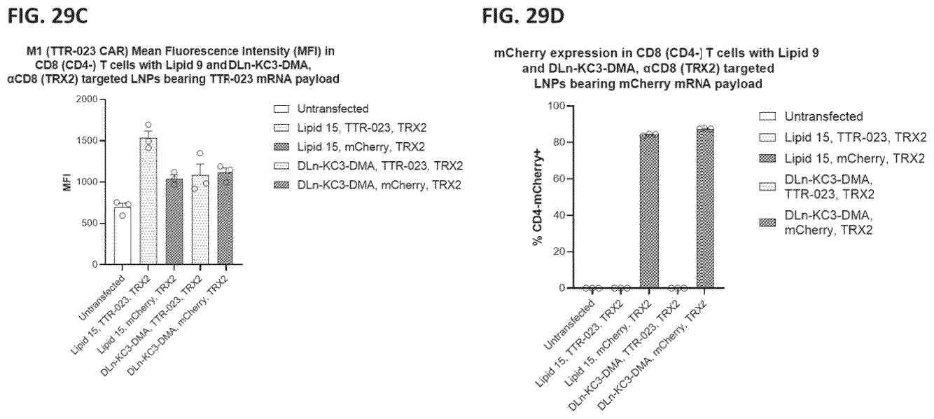

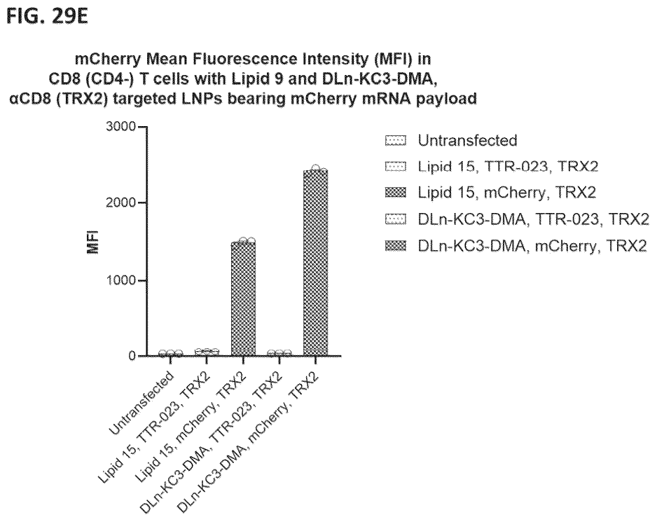

This example compares reporter protein (mCherry) and αCD20 CAR (TTR-023) protein expression resulting from LNP's derived from Lipid 15 to comparator Lipid DLin-KC3-DMA. Nanoparticles bearing mCherry mRNA or an αCD20 CAR encoding mRNA (TTR-023) were produced using the microfluidic mixing and buffer exchange processes described in Examples 2 and 6. An αCD8 Fab-conjugate (TRX2) was incorporated into the parent LNPs to obtain the final Antibody targeted LNP formulation using the process described in Example 5. Particles thus produced were tested in vitro in primary human CD3+ T cells to assess protein (mCherry or CAR) expression via mCherry fluorescence or detection of an M1 tag on the extracellular domain of the TTR-023 transmembrane CAR protein, respectively. Transfected cells were gated as CD4+ (CD4 population) and CD4−(CD8 population) and levels of protein expression monitored (as % M1+ and M1 MFI for CAR expression or reporter protein fluorescence for mCherry expression) in the two populations. Lipid 15 LNPs outperformed DLin-KC3-DMA LNPs in CAR expression levels (FIG. 29B and FIG. 29C) while DLin-KC3-DMA LNPs outperformed Lipid 15 LNPs in mCherry expression levels (FIG. 29D and FIG. 29E) suggesting that different optimal lipid compositions may be required for expression of intracellular proteins versus membrane bound proteins. Both TRX2 targeted lipid formulations with either CAR or mCherry payloads were well tolerated by primary human T-cells (FIG. 29A).

### Example 42. In Vitro CAR-T Cell Function by Raji (B-Cell) Co-Culture with aCD20 CAR (TTR-023) Expressing T-Cells Derived by Transfection of Primary Human T-Cells (of Example 29) with aCD8 (TRX2) Targeted Lipid 15 and DLin-KC3-DMA LNPs Bearing CAR-mRNA or mCherry-mRNA (as Negative Control), Bite (as Positive Control)

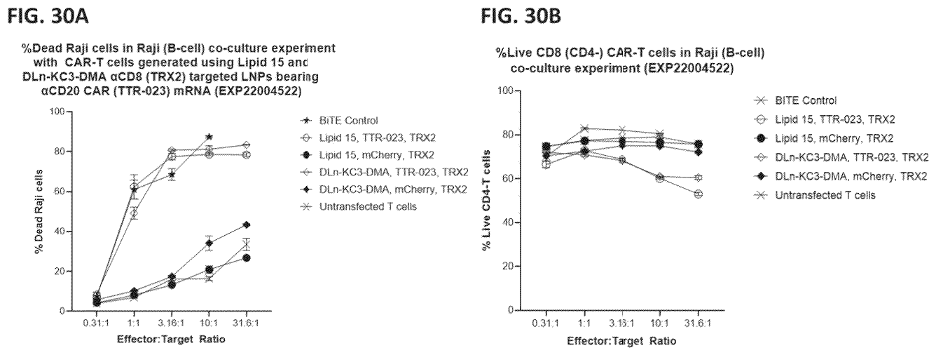

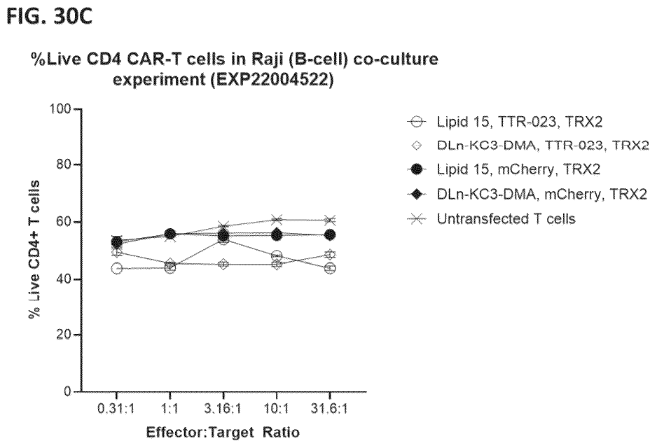

CAR-T cells produced in Example 40 were co-cultured with Raji (B-cells) at Effector: Target (E:T) (T-cell:B-cell) ratios of 0.31:1, 1:1, 3.16:1, 10:1, and 31.6:1 for a period of 24 hours and the fraction of Live B-cells and T-cells measured using the protocol described in Example 20. As seen in FIG. 30A, the fraction of dead B-cells increased at higher E:T ratios in a dose dependent manner upto an E:T of 3.16:1 and plateaued at E:T ratios indicating strong cytotoxic activity at an E:T of 3.16:1. Furthermore, T-cells expressing TTR-023 CAR protein exhibited significantly higher cytotoxicity towards B-cells relative to mCherry transfected T-cells as indicated by a 4× higher % of dead Raji cells for E:T ratios of 3.16 and below. This suggests that CAR engagement to target cell CD20 receptor and the downstream target specific granzyme perforin apoptotic pathway plays a major role in the observed T-cell activity while T-cell activation (possibly resulting for CD8 receptor engagement by the TRX2 antibody) over background levels of T-cell cytoxicity towards B-cells is a minor contributor in the overall activity of the CAR-T cells observed. Both Lipid 15 and DLin-KC3-DMA LNP formulations were equally cytotoxic towards B-cells exhibiting similar activity to the Bi-specific B-cell Engager (BiTE, bispecific antibody) positive control as seen in FIG. 30A. Both Lipid 15 and DLin-KC3-DMA formulations were well tolerated up to an E:T ratio of 3.16:1 with lower T-cell viability values observed in the CD8 T-cell population at the higher E:T ratios of 10:1 and 31.6:1 as seen FIG. 30B and FIG. 30C, respectively.

### Example 43. In Vitro Protein Expression (GFP) and LNP Association (Measured as DiI-Dye Fluorescence) in Primary Human T-Cells of aCD3 Targeted Lipids 15, 9, 10, and 13 and Comparator (DLin-KC3-DMA) LNPs Stored at 4° C. and After 1 Freeze-Thaw Cycle (Post −80° C. Storage)

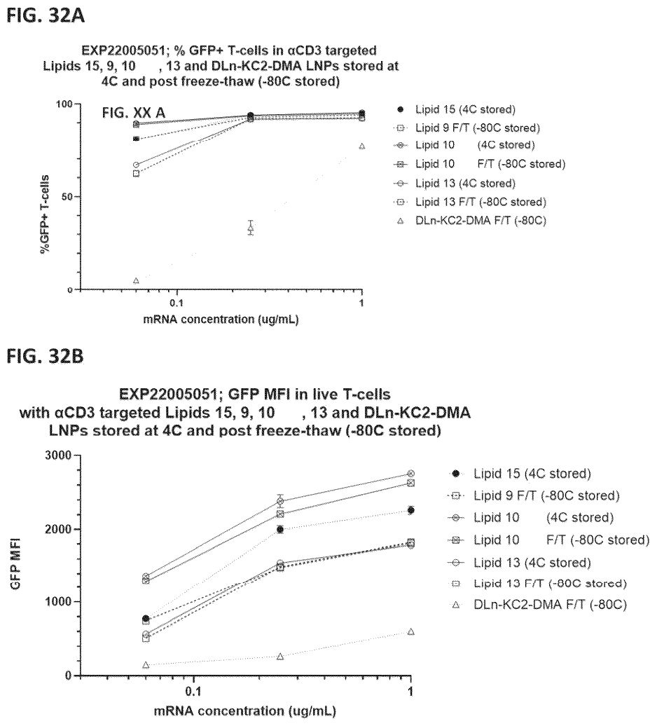

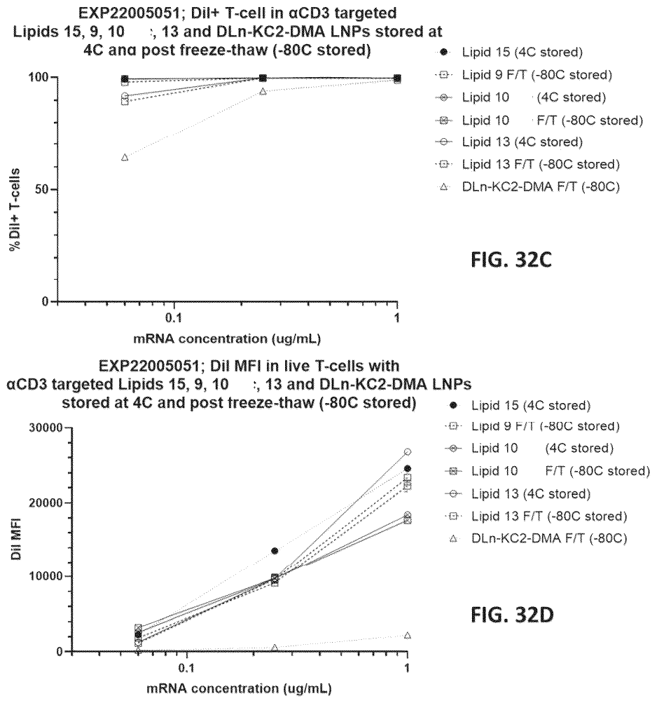

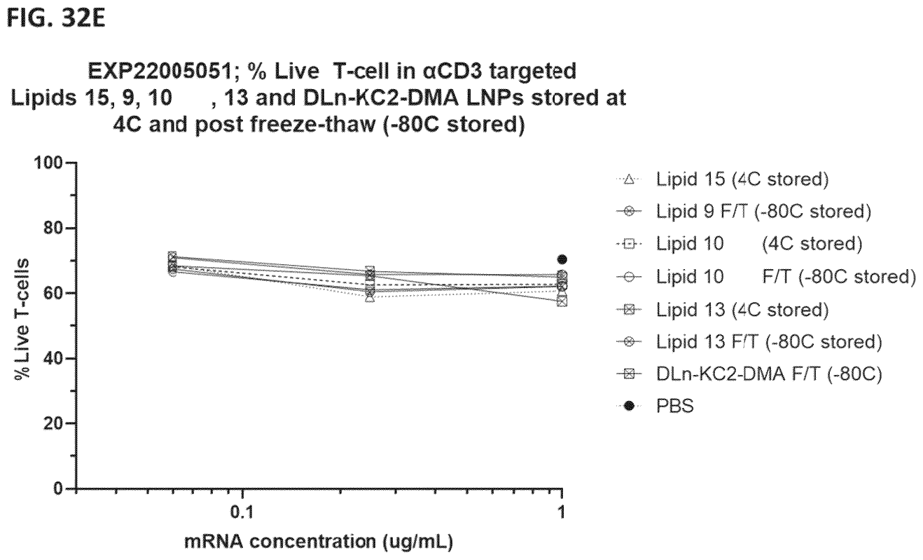

This example compares the GFP protein expression resulting from LNP's derived from Lipids 15, 9, 10 and comparator lipid DLin-KC2-DMA LNPs. Nanoparticles bearing GFP encoding mRNA (and optionally a fluorescent dye label (DiI-C18-5DS)) were produced using the microfluidic mixing and buffer exchange processes described in Example 2 and 6. An αCD3 Fab-conjugate was incorporated into the parent LNPs to obtain the final Antibody targeted LNP formulation using the process described in Example 5. Particles thus produced were tested in vitro in primary human CD3+ T cells to assess reported gene expression. As shown in FIG. 32A and FIG. 32B, at all dose levels, Lipid 15 and 10 LNPs performed similarly both in terms of fraction of GFP+ T-cells (reflected by % GFP+ cells) and the copies of GFP protein produced on a per cell basis (reflected by GFP MFI values) and significant better relative to Lipid 9 LNPs with respect to the copies of GFP protein produced on a per cell basis (reflected by GFP MFI values) particularly at the lower dose levels. Thus, improvements over lipid 9 performance could be achieved either by modification of the O-acyl substituent (from Linoleoyl of Lipid 9 to Oleoyl of Lipid 15) or by modification of N-acyl substituent (from the succinic acid derived 14 carbon N-acyl substituent of Lipid 9 to the succinic acid derived 12 carbon N-acyl substituent of Lipid 10). Additionally, as seen in FIGS. 32C and D, Lipid 9 and Lipid 13 LNPs exhibited similar cell association levels (both in terms of fraction of DiI+ cells and in terms of the copies of cell associated LNPs reflected by DiI MFI values shown in FIG. 32C and FIG. 32D) at all dose levels, however, Lipid 9 outperformed Lipid 13 LNPs in terms of protein expression both in terms of fraction of GFP+ T-cells (reflected by % GFP+ cells) and the copies of GFP protein produced on a per cell basis (reflected by GFP MFI values) suggesting inferior endosomal escape capabilities of Lipid 13 LNPs relative to Lipid 9 LNPs. The performance of Lipids 10 and 13 LNPs was well preserved after 1 Freeze Thaw cycle (Post −80° C. storage) as illustrated by comparison of % GFP+ cells and GFP MFI values before (4° C. stored) and after −80° C. storage as seen in FIG. 32A and FIG. 32B.

### Example 44. In Vivo T-Cell Reprogramming Using GFP Reporter Protein and a-CD8 (TRX-2) Targeted LNPs Based on Lipids 9, 15 and Comparator Lipid DLin-KC3-DMA (and Formulated with 1.5 Mol % DPG-Peg) in Human T-Cell Engrafted NSG Mice

NSG Mice were dosed using protocols described in Example 22. Plasma samples were drawn immediately prior to sacrificing the animals and spleen and liver harvested for analysis 24 hours post injection following the study design shown in Table 25.

TABLE 25
NSG Mice GFP T-Cell Reprogramming study design

| | | Formulation | | Targeting |
|---|---|---|---|---|
| Groups | Mice # | Dosed | Payload | Antibody |
| 1 | 2 | Buffer Control | NA | NA |
| 2 | 4 | Lipid 15; 1.5% | GFP-mRNA | TRX-2 |
| | | DPG-PEG; DiI | | |
| | | dye labelled | | |
| 3 | 4 | DLin-KC3-DMA; | GFP-mRNA | TRX-2 |
| | | 1.5% DPG-PEG; | | |
| | | No dye label used | | |
| 4 | 3 | Lipid 9; 1.5% | GFP-mRNA | TRX-2 |
| | | DPG-PEG; DiI | | |
| | | dye labelled | | |

CD4 and CD8 T-cells were stained sorted (by FACS) in blood, spleen and liver samples, liver samples were additionally stained and sorted for hepatocytes, endothelial cells, Kupffer cells, mouse macrophages and mouse myeloid cells using the Flow Panel depicted in Table 26 and Table 27. GFP fluorescence and Dil Dye fluorescence was used for quantitation of GFP protein expression (expressed as % GFP+ Cells and Mean Fluorescence Intensity (MFI) of GFP+ Cells) and LNP association (via DiI label fluorescence, expressed as % DiI+ cells and DiI-MFI of DiI+ Cells) in the stated cell types of interest.

TABLE 26
Cell Markers (Dead-Live, LNP, Protein, HuCD45,
huCD3, huCD4) and Fluorophores used

| Markers | Dead live stain | LNP (Dil dye) | GFP -Protein | huCD45 | huCD3 | huCD4 |
|---|---|---|---|---|---|---|
| Fluorophore | e-flour780 | APC | Green | BUV395 | BUV805 | BV711 |
| | | | Fluorescence | | | |

TABLE 27
Cell markers (Dead-Live, huCD8, muCD45, hu/muCD11b,
muCD31, F4/80) and Fluorophores used

| Markers | Dead live stain | huCD8 | ARRUCD45 | Hu/mmCD11b+ | RWCD31 | F4/80 |
|---|---|---|---|---|---|---|
| Fluorophore | e-flour780 | BV421 | BB700 | BV785 | BUV737 | PE Dazzle |

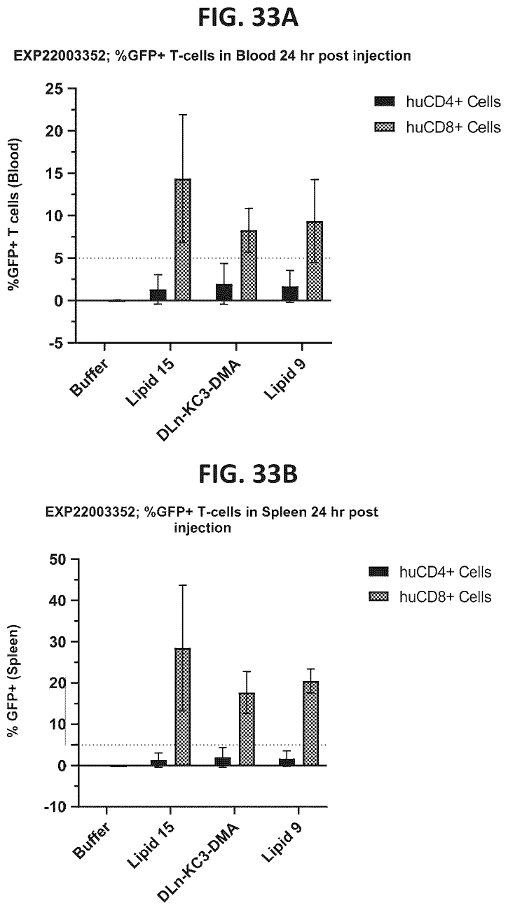

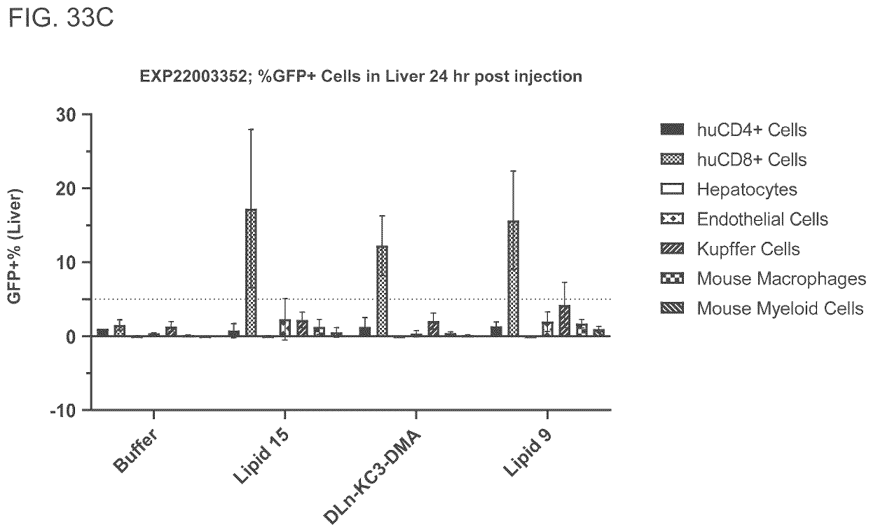

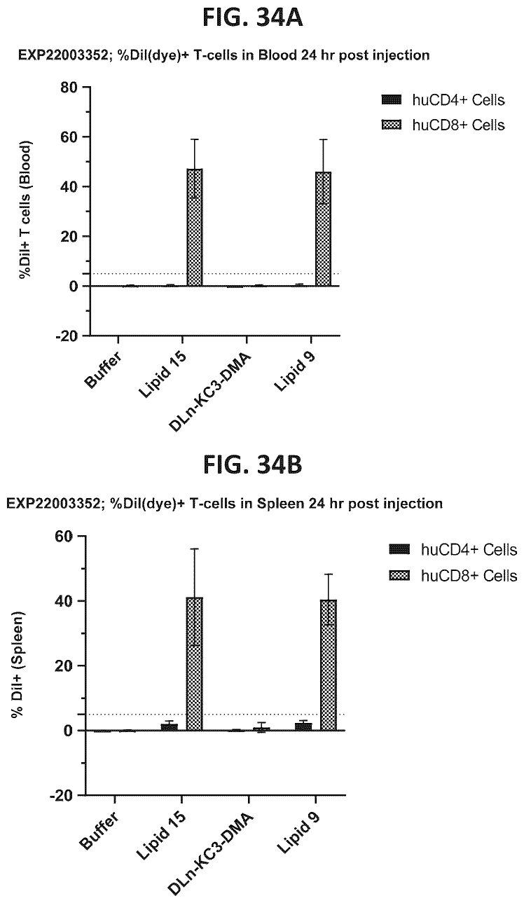

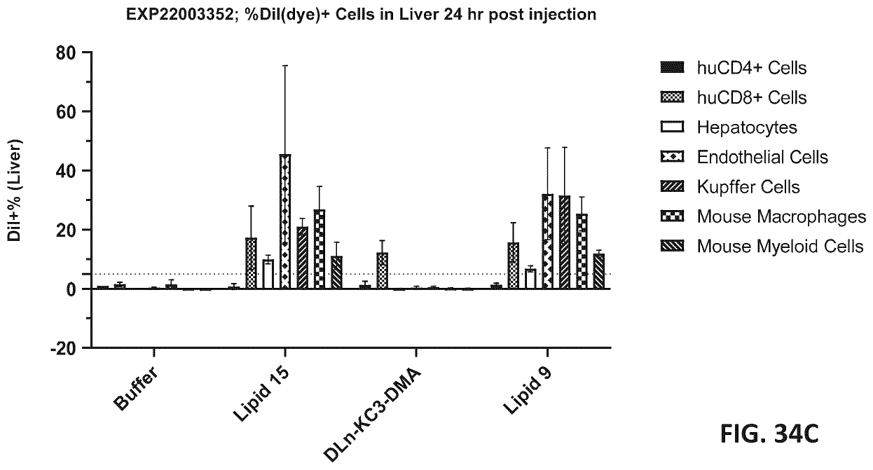

As seen in FIGS. 33A, 33B, and 33C, 7-25% GFP+ cells were detected in the CD8 T-cell population in blood, spleen and liver samples, while <3% of the CD4+ T-cells were GFP+ confirming differential in vivo reprogramming of the CD8+ T-cell population and LNP targeting using TRX-2 αCD8 antibody. As seen in FIGS. 34A, 34B, and 34C, LNP association was specific to the CD8 T-cell population in blood and spleen samples, however, significant levels of association to the CD4 population as well as endothelial cells, Kupffer Cells, and mouse macrophages was observed in the liver samples. Notably, despite non-specific LNP association, no GFP protein was detected (FIG. 33C) indicating that off target LNP association does not result in off-target mRNA delivery and protein expression.

### Example 45. Preparation of LNPs by Microfluidic Mixing Using Exemplary Lipids

This Example describes the production of mRNA-loaded LNPs using exemplary materials and microfluidic mixing process.

LNPs encapsulating an mRNA payload were prepared by mixing an aqueous mRNA solution and an ethanolic lipid blend solution (containing the ionizable lipid Lipid 15, DSPC, DPG-PEG and Cholesterol at lipid ratios shown in TABLE 28) using an in-line microfluidic mixing process. The mRNA stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in TABLE 28 below.

TABLE 28

| | | Ratio of Lipid | Concentration | Theoretical |
|---|---|---|---|---|
| | | to mRNA (nmol | in Lipid | LNP Lipid |
| | | lipid/100 μg | Solution | Composition |
| Lipid | Source | mRNA) | (mM) | (mol %) |
| Lipid 15 | — | 1,500 | 18 | 49.2449 |
| Cholesterol | Dishman | 1,200 | 14.4 | 39.3959 |
| | Netherlands | | | |
| DSPC | Avanti Polar | 300 | 3.6 | 9.849 |
| | Lipids, Alabama, | | | |
| | U.S. | | | |
| DPG-PEG(2000) | NOF America, | 46 | 0.55 | 1.5102 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed using the NanoAssemblr Ignite (the ratios, volumes, and flow rates can vary). The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with MBS exchange buffer. The LNP suspension (5 mL) was then loaded into the device and centrifuged at 500 RCF until the volume was reduced by half (2.5 mL). The suspension was then diluted with exchange buffer (2.5 mL of MBS) to bring the suspension back to the original volume. This process of two-fold concentration and two-fold dilution was repeated five additional times for a total of six discontinuous diafiltration steps. The retentate containing the LNPs in MBS was recovered from the centrifugal ultrafiltration device, mixed with sucrose to a final sucrose concentration of 10% w/v, and then filtered using a 0.2 μm PES syringe filter. The LNPs were either used right away, or stored frozen at −80° C. until further use.

### Example 46. Characterization of LNPs

This Example describes the characterization of LNPs produced in Example 45.

Samples of the LNPs produced in Example 45 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both un-encapsulated RNA and accessible RNA at the LNP surface, is measured by diluting the nanoparticles to approximately 1 μg/mL mRNA using HEPES buffered saline, and then adding Quant-iT reagent to the mixture. Total RNA content is measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA is quantified by measuring fluorescence at 485/535 nm, and concentration is determined relative to a contemporaneously run RNA standard curve. The results are set forth in TABLE 29.

TABLE 29

| | | DLS | | Zeta | Zeta | Dye- |
|---|---|---|---|---|---|---|
| | | Z-Avg. | | Potential | Potential | Accessible |
| | | Diameter | DLS | at pH 5.5 | at pH 7.4 | mRNA |
| LNP Lot Number | mRNA Payload | (nm) | PDI | (mV) | (mV) | (%) |
| EXP23012510- | CD22 CAR | 87 | 0.10 | 21.9 | 1.2 | 22 |
| N77S | (TTR-102 (SEQ | | | | | |
| | ID 147)) | | | | | |
| EXP23012510- | CD22 CAR | 89 | 0.08 | 21.3 | 0.9 | 16 |
| N80S | (TTR-121 (SEQ | | | | | |
| | ID 148)) | | | | | |
| EXP23012510- | mCherry | 89 | 0.10 | 20.1 | 0.8 | 13 |
| NCS | | | | | | |

### Example 47. Preparation of FAB Or VHH Conjugates to Enable T-Cell Targeting

Fab or VHH targeting moieties were conjugated to DSPE-PEG (3.4k)-maleimide via covalent coupling between the maleimide group and a C-terminal cysteine in the heavy chain (HC). The protein (3-4 mg/mL), after buffer exchange into pH 7.4 phosphate buffered saline (PBS 137 mM NaCl, 2.7 mM KCl, 10 mM Na₂HPO₄, and 1.8 mM KH₂PO₄) with 5 mM ethylenediaminetetraacetic acid (EDTA), was reduced using 2 mM Tris(2-carboxyethyl)phosphine hydrochloride (TCEP) for 1 hour at room temperature. The reduced protein was isolated using a 7 kDa molecular weight cutoff (MWCO) SEC column to remove TCEP and buffer exchanged into fresh PBS with 5 mM EDTA.

The conjugation reaction was initiated by addition of a 10 mg/mL micellar suspension of DSPE-PEG-maleimide and 30 mg/mL DSPE-PEG-OCH3 (1:1 to 1:4 weight ratio is used depending on protein) in PBS. The conjugation reaction is carried out using 2-4 mg/mL protein and a 3.5 molar excess of maleimide at 37° C. for 2 hours followed by incubation at room temperature for an additional 12-16 hours.

The production of the resulting conjugate was monitored by HPLC and the reaction quenched in 2 mM cysteine. The resulting conjugate (DSPE-PEG-Fab) is isolated using a 100 kDa or 50 kDa MWCO Millipore Regenerated Cellulose membrane filtration using pH 7.0 HEPES buffer saline (25 mM HEPES, 150 mM NaCl) and stored at 4° C. prior to use. After quenching, the final micelle composition consists of a mixture of DSPE-PEG-Fab, DSPE-PEG-maleimide (cysteine terminated), and DSPE-PEG-OCH3. The ratio of the three components is approximately DSPE-PEG-Fab: DSPE-PEG-maleimide (cysteine terminated): DSPE-PEG-OCH3=1:2.45:3.45-10.35 (by mol)).

Generation of Nb-conjugates follows the same procedure but uses a 1:1:4 or 1:2:3 (VHH:DSPE-3.4K PEG-maleimide: DSPE-2KPEG-OCH3) molar ratio, as described in later examples.

### Example 47-1. Preparation of FAB OR VHH Conjugates with Shortened DSPE-PEG Linker

Fab or VHH targeting moieties were conjugated to DSPE-PEG (2k)-maleimide via covalent coupling between the maleimide group and a C-terminal cysteine in the heavy chain (HC) as described in Example 47.

### Example 47-2. Preparation of DSPE-PEG(3.4K)-15C01V8 Conjugate

15C01v8 (SEQ ID 9, SEQ ID NO: 169, or SEQ ID NO: 44) was conjugated to DSPE-PEG (3.4k)-maleimide via covalent coupling between the maleimide group and a C-terminal cysteine in the heavy chain (HC) as described in Example 47.

### Example 48. Preparation of LNPs Containing T-Cell Targeting Group

This Example describes the incorporation of an immune cell targeting conjugate into preformed LNPs.

LNPs from Example 45 and DSPE-PEG-anti-CD8 VHH conjugate 15C01v8 from Example 47-2 (SEQ ID 9, SEQ ID NO: 169, or SEQ ID NO: 44) (prepared using the methods described in Example 104) were combined in a 15 ml conical tube at a ratio of 0.084 g V_{HH} conjugate per 1 g of mRNA and diluted to a final mRNA concentration of 0.2 mg/mL. The tube was placed into a ThermoMixer pre-heated to 37° C. and then mixed at 300 rpm for 4 hours at 37° C. The resulting targeted LNP suspension was subsequently filtered using a 0.2 μm PES syringe filter and then either used immediately or stored frozen at −80° C.

### Example 49. Preparation of LNPs by Microfluidic in-Line Mixing and TFF Using an Exemplary Ionizable Lipid

This example describes preparation of LNPs using scalable unit operations, namely in-line microfluidic mixing followed by tangential flow filtration (TFF) for ethanol removal and buffer exchange.

LNPs encapsulating an mRNA payload were prepared by mixing an aqueous mRNA solution and an ethanolic lipid blend solution using an in-line microfluidic mixing process. The mRNA (CD22 CAR TTR-102 (SEQ ID NO: 139) or TTR-102 (SEQ ID 147)) stock solution was diluted in pH 4 acetate buffer (yielding a 400 μg/mL solution of mRNA) in 65 mM pH 4 acetate buffer. The lipid components were dissolved in anhydrous ethanol at the relative ratios set forth in TABLE 30 below.

TABLE 30

| | | Ratio of Lipid | Concentration | Theoretical |
|---|---|---|---|---|
| | | to mRNA (nmol | in Lipid | LNP Lipid |
| | | lipid/100 μg | Solution | Composition |
| Lipid | Source | mRNA) | (mM) | (mol %) |
| Lipid 15 | — | 1,500 | 18 | 49.2449 |
| Cholesterol | Dishman | 1,200 | 14.4 | 39.3959 |
| | Netherlands | | | |
| DSPC | Avanti Polar | 300 | 3.6 | 9.849 |
| | Lipids, Alabama, | | | |
| | U.S. | | | |
| DPG-PEG(2000) | NOF America, | 46 | 0.55 | 1.5102 |
| | New York, U.S. | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directly mounted into the luer ports of the mixing cartridge. The two solutions were then mixed at a 3:1 v/v ratio of mRNA solution (9 mL) to lipid solution (3 mL) at a total flow rate of 9 mL/min using the NanoAssemblr Ignite. This mixing process was performed three additional times, and the resulting LNP suspensions were pooled to yield 48 mL of LNP suspension at 0.3 mg/mL. The resulting suspension was held at room temperature for a minimum of 5 minutes before proceeding to ethanol removal and buffer exchange. Ethanol removal and buffer exchange were subsequently performed using constant volume tangential flow filtration (TFF).

Following mixing, ethanol removal and buffer exchange were performed on the resulting LNP suspension using a hollow fiber TFF module (mPES membrane with 300 kDa MWCO, Repligen, US) and MBS as the exchange buffer. Briefly, the TFF module was rinsed with DI water and pumped dry before use. The LNP mixture in ethanol/water solution was added to the feed reservoir, the TFF module was primed, and diafiltration with MBS was initiated by ramping up the peristaltic pump to the target flow rate and adjusting the retentate valve until the target transmembrane pressure (TMP) was reached. A shear rate of 8000 s⁻¹ and a TMP of 3.5 psi were the target operating parameters for the system once diafiltration was initiated. Throughout the diafiltration process, the TMP was kept constant by adjusting the retentate valve. Permeate flux was monitored and was >20 LMH throughout diafiltration. Six diafiltration exchange volumes were performed, with samples set aside at the end of each diafiltration volume to later track the buffer exchange process. Final ethanol content was ≤0.8%, as measured by refractive index measurements on permeate samples, and pH measurements confirmed the buffer exchange into the desired diafiltration buffer. Upon the completion of six diafiltration volumes, a concentration of the resulting LNP suspension was subsequently performed.

The concentration of the LNP suspension to a target total mRNA concentration of ~0.8 mg/mL was performed using the same TFF module that was used during the buffer exchange process. TMP and flow rate during the buffer exchange process were maintained, and the suspension was allowed to concentrate by stopping the addition of diafiltration buffer to the retentate reservoir. The resulting LNP suspension was collected and filtered with a 0.2 μm syringe filter. The suspension was then mixed with a sterile-filtered 49 wt % sucrose solution to a final sucrose concentration of 10% w/v and then stored frozen at −80° C.

Using the LNP characterization process in Example 46, the LNP batch was characterized to determine the average hydrodynamic diameter, zeta potential at pH 5.5 and 7.4, and mRNA content (total and dye-accessible). As seen in TABLE 31, the microfluidic mixing process with ethanol removal and buffer exchange by TFF results in ~100 nm particles exhibiting narrow polydispersity and good mRNA encapsulation (<10% dye accessible RNA).

TABLE 31

| | DLS | | Zeta | Zeta | | Dye- |
|---|---|---|---|---|---|---|
| | Z-Avg. | | Potential | Potential | Total | Accessible |
| | Diameter | DLS | at pH 5.5 | at pH 7.4 | mRNA | mRNA |
| LNP Lot Number | (nm) | PDI | (mV) | (mV) | (mg/mL) | (mg/mL) |
| EXP22015234-NX501 | 97 | 0.11 | 22.0 | −0.4 | 0.61 | 0.05 |

### Example 50. Physiochemical Properties (Pre- and Post-Insertion with AntiCD8 VHH Conjugate 15C01V8 (SEQ ID 9)) of Lipid 15 CAR (TTR-102 (SEQ ID NO: 139) or TTR-102 (SEQ ID 147)) RNA LNPs

LNPs from Example 48 inserted with DSPE-PEG-anti-CD8 V_{HH} conjugate 15C01v8 (SEQ ID 9) were characterized as described in Example 46. The post-insertion characterization results are summarized in TABLE 32. Compared against the pre-insertion results summarized in TABLE 29, the insertion process led to a size increase of ~10 nm, a zeta potential decrease of ~3 mV, and a reduction in the dye-accessible mRNA content to ~5%.

TABLE 32

| | | DLS | | Zeta | Zeta | Dye- |
|---|---|---|---|---|---|---|
| | | Z-Avg. | | Potential | Potential | Accessible |
| | | Diameter | DLS | at pH 5.5 | at pH 7.4 | mRNA |
| LNP Lot Number | mRNA Payload | (nm) | PDI | (mV) | (mV) | (%) |
| EXP23012510- | CD22 CAR | 97 | 0.12 | 18.9 | −0.7 | 5.4 |
| N77T | (TTR-102 (SEQ | | | | | |
| | ID 147)) | | | | | |
| EXP23012510- | CD22 CAR | 98 | 0.10 | 21.0 | −1.9 | 4.7 |
| N80T | (TTR-121 (SEQ | | | | | |
| | ID 148)) | | | | | |
| EXP23012510- | mCherry | 96 | 0.10 | 18.6 | −2.0 | <5.0 |
| NCT | | | | | | |

### Example 51. Method for the Primary Human CD8+ T-Cell Transfection with DiI-C18-5DS Labelled LNPs

CD8+ T cells were isolated from frozen peripheral blood mononuclear cells using an EasySep Human T Cell Isolation Kit on a RoboSep automated cell isolation system from STEMCELL. T cells were plated into a round bottom 96-well plate in RPMI cell culture media supplemented with glutamax, 10% fetal bovine serum, and 50 ng/ml IL-2. 100 μL of cell suspension was seeded per well at a density of 1M T cells/mL (100K T cells/well). Cells were allowed to rest overnight in a 37° C. incubator, 5% CO2, and then transfected by gently adding 10 μL of a 11 μg/mL (by mRNA) nanoparticle suspension, resulting in a final mRNA concentration of 1 μg/mL (unless otherwise noted). Cells were gently mixed with a pipette and then incubated for 24 hours in a 37° C. incubator, 5% CO₂. After incubation, the cells were diluted with FACS buffer (BD 554657) and analyzed using a Novocyte Penteon Flow Cytometer (Agilent). Data were analyzed using FlowJo software from BD biosciences.

### Example 52. CD4 and CD69 Staining Protocol Post Transfection

After 24 hours, cells were transferred to a 96-well conical bottom polypropylene plate and centrifuged at 350×g for 5 minutes. Supernatants were removed and transferred to a fresh conical bottom polypropylene plate for further cytokine analysis. Cells were washed by adding 200 μL FACS buffer (BD 554657), centrifuging at 350×g for 5 min, and then aspirating the supernatant from each well. BV421 anti-human CD69 (BioLegend 310930 clone FN50) and BV711 anti-human CD4 (BioLegend 344648 clone SK3) antibodies were diluted 100-fold by adding 100 μL of each antibody to 10 mL FACS buffer. 100 μL of the diluted antibody solution was added to each well and the plate was incubated at 4° C. for 30 minutes. The plates were then washed by centrifuging at 350×g for 5 min, removing the supernatant, re-suspending in 200 μL FACS buffer, centrifuging at 350×g for 5 min, and aspirating the supernatant from each well. Following the wash, cells were resuspended in 100 μL of FACS and immediately analyzed by flow cytometry. Flow cytometry analysis was performed using a Novocyte Penteon (Agilent). Data were analyzed using FlowJo software from BD biosciences.

### Example 53. Method for Quantifying Secreted Cytokines by MSD

IFN-γ, TNF-alpha, Granzyme B, Granzyme A, and GM-CSF were assayed using a custom U-Plex MSD panel (Meso Scale Discovery). Briefly, 200 μL of each biotinylated antibody was added to 300 μL of the corresponding assigned linker and mixed by vortexing. After 30 minutes of incubation at room temperature, 200 μL of stop solution was added and the solution was mixed by vortexing. 600 μL of each U-PLEX Linker-coupled antibody solution was combined into a single tube and mixed by vortexing. The total volume was brought up to 6 mL with Stop solution to reach a final 1× concentration. 50 μL of the prepared multiplex coating solution was added to each well in the U-PLEX 96-well plate and the plate was sealed and incubated at room temperature with shaking (700 rpm) for one hour. Following coating, the plate was washed 3 times using 150 μL/well of 1×MSD Wash Buffer. Calibrator standards were prepared according to the manufacturer's instructions using a 4-fold dilution schema. 50 μL of sample supernatants (diluted 3-fold) and calibrator standards were added to the respective wells. The plate was sealed and incubated at room temperature with shaking (700 rpm) for 2 hours. The plate was then washed 3 times using 150 μL/well of 1×MSD Wash Buffer. Detection antibody stock was diluted 100× to reach a 1× concentration and 50 μL was added to each well following 1 hour incubation at room temperature with shaking (700 rpm). The plate was then washed 3 times using 150 μL/well of 1×MSD Wash Buffer and 150 μL of MSD GOLD Read Buffer was added to each well before immediately analyzing using a MESO QuickPlex SQ 120 MM instrument. The cytokine concentrations were quantified relative to the respective standard curves. The data was analyzed using the MSD Discovery Workbench software and plotted in Graph Pad Prism.

### Example 54. CD22 CAR Sandwich Staining Protocol

Cells were transferred to a 96-well conical bottom plate and washed by centrifuging at 350×g for 5 minutes followed by resuspension in FACS buffer (BD 554657). CD22 CAR protein was detected by first staining with biotinylated CD22 Ag (Acro Biosystems SI2-H82E3) at 4° C. for 30 minutes followed by washing twice in FACS buffer and then staining with anti-CD22 antibody conjugated to APC (eBioscience 17-0229-42). Following staining, cells were washed twice by centrifugation at 350×g for 5 minutes followed by resuspension in FACS buffer. Cells were analyzed by FACS using a Novocyte Penteon (Agilent).

### Example 55. CD22 CAR Primary Staining Protocol

Cells were transferred to a 96-well conical bottom plate and washed by centrifuging at 350×g for 5 minutes followed by resuspension in FACS buffer (BD 554657). CD22 CAR protein was detected by a single staining with CD22 Ag conjugated to FITC (Acro Biosystems SI2-HF2H6) at 4° C. for 30 minutes followed by washing twice in FACS buffer and then resuspending in FACS buffer. Cells were analyzed by FACS using a Novocyte Penteon (Agilent).

### Example 56. In Vitro LNP CAR Primary Human T-Cell Transfection and Cancer B-Cell Line Co-Culture for Assessment of CAR-T Cell Function

CD8+ T cells were isolated from human PBMCs by using EasySep Human CD8+ T cell isolation kit (catalog #17953) and EasySep Human T cell isolation kit (catalog #17951) according to manufacturer's instructions. Isolated T cells were rested overnight in T cell media (RPMI+Glutamax, Gibco, catalog #61870-036) with 10% heat inactivated fetal bovine serum (HI—FBS, Gibco, catalog #16140-071) supplemented with 100 IU human IL-2 (Miltenyi catalog #130-097-748) at a density of 1,000,000 cells/well in a 12 well plate. T cells were then transfected with LNPs at a mRNA concentration of 0.3 μg/ml. CAR expression was detected 24 h post LNP transfection by flow cytometry. 4 hours post-transfection and before co-culture with B-cell cancer cell lines (Raji WT, Raji CD22KO, Nalm6 WT, Nalm6 CD22KO, Daudi, K562, JVM-2, or Reh), the transfected T cells were washed twice with T cell media. B-cell cancer cells were stained with cell trace violet (CTV, Thermo Fisher, catalog #C34571) and co-cultured with different effector to target (E:T) ratios of T cells in a 96 well flat-bottom plate format and incubated at 37° C. for 48 hours. 48 hours post co-culture the cells were stained with live/dead stain (eBioscience Fixable viability dye eFluor 780, Invitrogen catalog #2290917) and analyzed by flow cytometry. Data was analyzed with FlowJo and Graph Pad prism.

### Example 57. Method for Primary NHP T-Cell Transfection with Car LNPs

Rhesus CD8+ T cells were isolated from frozen rhesus peripheral blood mononuclear cells using an EasySep NHP T Cell Isolation Kit on a RoboSep automated cell isolation system from STEMCELL. T cells were plated into a round bottom 96-well plate in RPMI cell culture media supplemented with glutamax, 10% fetal bovine serum, and 50 ng/ml human IL-2. 100 μL of cell suspension was seeded per well at a density of 1M T cells/mL (100K T cells/well). Cells were allowed to rest overnight in a 37° C. incubator, 5% CO₂, and then transfected by gently adding 10 μL of a 11 μg/mL (by mRNA) nanoparticle suspension, resulting in a final mRNA concentration of 1 μg/mL (unless otherwise noted). Cells were gently mixed with a pipette and then incubated for 24 hours in a 37° C. incubator, 5% CO₂. After incubation the cells were diluted with FACS buffer (BD 554657) and analyzed using a Novocyte Penteon Flow Cytometer (Agilent). Data were analyzed using FlowJo software from BD biosciences.

### Example 58. Method for Transfecting HEK293T Cells with CAR Plasmid Via Lipofectamine

HEK293T cells were seeded in DMEM media into a 48-well plate to reach 70-90% confluency at the time of transfection. 30 minutes prior to transfection the media was replaced with Opti-MEM. For each reaction, 0.6 μL Lipofectamine 3000 Reagent was diluted in 20 μL Opti-MEM Medium and vortexed. 0.4 μg of CAR plasmid DNA was diluted in 20 μL Opti-MEM and 0.8 μL of P3000 reagent was added. 20 μL of the diluted CAR plasmid DNA solution was added to 10 μL of the diluted Lipofectamine 3000 reagent. The solution was then incubated for 15 minutes at room temperature before adding 10 μL of the DNA-lipid complex solution to the respective well containing HEK293T cells. Transfected HEK293T cells were incubated for 24 hours at 37 C until analysis by flow cytometry.

### Example 59. Jurkat Transfection with Car Plasmid Via Electroporation

Jurkats were harvested from culture and counted. For each reaction, 100K Jurkat cells were resuspend in 20 μL SE 4D-Nucleofector™ X Solution (Lonza) and 0.6 μg CAR plasmid DNA was added to the cells and co-incubated for 3 minutes at room temperature. The solution was then transferred to a 16-well electroporation strip (Lonza) and electroporated using the CL-120 pulse code on a 4D-Nucleofector system (Lonza). Following electroporation, cells were immediately recovered in 180 μL pre-warmed recovery media (RPMI+20% FBS), seeded into a 96-well plate at 20K cells per well, and incubated over night at 37 C. Following recovery, transfected Jurkats were co-cultured with Raji cells at varying effector to target cell ratios (E:T) and then incubated at 37 C for 24 hours before staining and analyzing by flow as described in Example 52.

### Example 60. Transfection of Jurkat Cells with mRNA Via Electroporation

Jurkat cells were harvested from culture and counted. For each reaction, 100K Jurkat cells were resuspend in 20 μL SE 4D-Nucleofector™ X Solution (Lonza) and 0.6 μg CAR mRNA was added to the cells and co-incubated for 3 minutes at room temperature. The solution was then transferred to a 16-well electroporation strip (Lonza) and electroporated using the CL-120 pulse code on a 4D-Nucleofector system (Lonza). Following electroporation, cells were immediately recovered in 180 μL pre-warmed recovery media (RPMI with 20% FBS), seeded into a 96-well plate at 20K cells per well, and incubated over night at 37 C. Following recovery, transfected Jurkats were co-cultured with Raji cells at varying effector to target cell ratios (E:T) and then incubated at 37 C for 24 hours before staining and analyzing by flow as described in Example 52.

### Example 61. Transfection of Primary Human T-Cells with mRNA Via Electroporation and Subsequent Co-Culturing with Target Cells

Primary human CD8+ T cells were isolated from frozen peripheral blood mononuclear cells using an EasySep Human T Cell Isolation Kit on a RoboSep automated cell isolation system from STEMCELL. T cells were plated into a 6-well plate in RPMI cell culture media supplemented with glutamax, 10% fetal bovine serum, and 50 ng/ml IL-2. 3 mL of cell suspension was seeded per well at a density of 1M T cells/mL (3 million T cells/well). Cells were allowed to rest overnight in a 37° C. incubator, 5% CO₂. For each reaction, 1 million T-cells were harvested and resuspended in P3 electroporation buffer (Lonza) and 1 μg of mRNA was then added to the cell suspension before transferring the cell-mRNA solution to an electroporation cuvette (Lonza). Cells were electroporated using the FI-115 pulse code on a 4D-Nucleofector system (Lonza). Following electroporation, cells were immediately recovered in 500 μL pre-warmed recovery media (RPMI with 20% FBS and 50 ng/mL IL-2), seeded into a 12-well plate and incubated at 37 C for 1 hour. For CAR expression studies, cells were allowed to rest for 24 hours until the time of analysis where cells were then stained as described in Example 54 and analyzed by flow cytometry using a Novocyte Penteon Flow Cytometer (Agilent). For cytotoxicity assays, electroporated T-cells were co-cultured with target Raji or Nalm6 cells at varying effector to target cell ratios (E:T) and then incubated at 37° C. for 48 hours before staining and analyzing by flow as described in Example 52.

### Example 62. NHP CD22 Staining Protocol

Cells were transferred to a 96-well conical bottom plate and washed by centrifuging at 350×g for 5 minutes followed by resuspension in FACS buffer (BD 554657). CD22 CAR protein NHP cross-reactivity was determined by first staining with his-tagged Cyno CD22 Ag (R&D 9864-SL) or his-tagged Rhesus CD22 Ag (Acro Biosystems SI2-R⁵²Ha) at 4° C. for 30 minutes followed by washing twice in FACS buffer and detecting with anti-HIS mAb-iFluor647 (Genscript A01802-100). Following staining, cells were washed twice by centrifugation at 350×g for 5 minutes followed by resuspension in FACS buffer. Cells were analyzed by FACS using a Novocyte Penteon (Agilent) and expression was compared to human CD22 CAR staining performed as described in Example 54.

### Example 63. Receptor Quantification with Quantibrite PE Beads

Cells were transferred to a 96-well conical bottom plate and washed by centrifuging at 350×g for 5 minutes followed by resuspension in FACS buffer (BD 554657). Cells were stained with anti-CD22-PE antibody (Biolegend 302506) at 4° C. for 30 minutes. Following staining, cells were washed twice by centrifugation at 350×g for 5 minutes followed by resuspension in FACS buffer. Cells were analyzed by FACS using a Novocyte Penteon (Agilent). Quantibrite PE beads (BD 340495) were reconstituted with 500 L FACS buffer and immediately analyzed on a Novocyte Penteon (Agilent) using the same PE voltage settings as for the cells. The number of receptors per cell was quantified relative to the standard curve based on contemporaneously analyzed Quantibrite PE beads.

### Example 64. Protocol for In Vivo CD8+ T-Cell Reprogramming in Human PBMC-Engrafted NSG-Mice

The following is a standard procedure for in-vivo reprogramming of immune cells with anti CD8-targeted LNPs encapsulating CAR-encoding mRNA.

### Mice Strains and Humanization

NSG or NSG-MHCI/II Ko mice were obtained 6-8 weeks old female mice were engrafted with 10 million PBMC from a donor with a pre-characterized donor profile for Immuno-Oncology studies via tail vein injection. Body weight was monitored twice a week and blood samples were collected at Day 10-12 post PBMC inoculation to evaluate PBMC engraftment.

### Evaluation of Human T-Cell Engraftment in Humanized-Mice

75-100 μl of blood was collected by submandibular bleed from each mouse. Red blood cells were lysed using Versalyse, RBC lysis solution following protocol as instructed by manufacturer (Beckman Coulter A09777). Cells were stained with mCD45, hCD45, hCD3, hCD8 and hCD4 to quantify the engraftment of human T-cells. Count Bright Absolute Counting Beads (Thermofisher Scientific) were added as instructed by manufacturer to determine the absolute counts of hCD8+ cells/μl. At day 10-12 days post PBMC injection, engraftment of huCD45+ is 10-25% of total gated alive cells. At day 14 days post PBMC injection (day when the first dose of LNP/mRNA is administered), engraftment of huCD45+ is 20-45% of total gated alive cells.

### In Vivo-Reprogramming of CD8+ T Cells to Anti-CD22 CAR-T

At day 14 post PBMC engraftment mice were treated with one single dose of LNP loaded with mRNA (CAR-T-construct or mCherry) via tail vein injection. For single-dose PD/PK studies mice were sacrificed at 6, 24, 48 or 72 hr post dosing and WB and tissues (Spleen, Lung, Bone Marrow (BM)) were collected to evaluate CAR-T expression and in vivo function. CAR T in vivo function was evaluated by the % of B cell aplasia detected in spleen and BM. B cell aplasia is a sign of functional CART persistence. For multiple-dose PD/PK studies mice were dosed every three days for a total of n=4 dose (2QW) or every 7 days (QW) for a total of n=3 dose. WB was collected after the first and third (QW) or fourth dose (2QW) for evaluation of CAR-T expression (at 6 and 24 hr post dosing). In addition, tissues (Spleen, Lung, BM) were harvested after the third (QW) or fourth (2QW) dose to evaluate CAR-T expression, biodistribution and function (at 6 or 24 hrs).

### Tissue and Blood Sample Collection

In life blood collection (PBMC engraftment check or CAR-T expression check in WB post Dose #1 in multiple dose studies) was performed via submandibular bleed. For submandibular blood collection, the mandibular vein was punctured by an appropriate sterile animal lanzet and blood was collected into an EDTA-tube (Sarstedt microvette 500).

Terminal blood collection was performed by cardiac puncture post CO2 euthanasia at above mentioned time points. Tissues (Lung, Spleen and Bone Marrow) were harvested and processed for hematopoietic cell isolation and flow cytometry analysis. Single cell preparations from spleen was generated by smashing the spleen with a syringe piston over a 70 μm filter. Bone marrow was isolated by flushing the femurs with PBS. Lung was processed to a single cell suspension using the Lung Dissociation Kit Mouse from Miltenyi (130-095-927) as indicated by manufacturer.

### Immunophenotyping Analysis

Immune cells from blood and all the above tissues were processed with Versalyse (WB) or ACK buffer (tissues) (Gibco, A1049201), RBC lysis buffer as per manufacturing instructions. Immune cells were stained with live/dead fixable dye and surface markers with standard flow analysis protocol as shown in below panel. BD symphony flow cytometer was used to determine CAR T expression and B cell aplasia. Count Bright Absolute Counting Beads (Thermofisher Scientific) were added as instructed by manufacturer to determine the absolute counts of hCD22+ CAR T cells/μl.

| | | | | | | Target | Mono- |
|---|---|---|---|---|---|---|---|
| Marker | Clone | Fluorophore | Vendor | Catalog # | Size | Species | Polyclonal |
| Live/Dead | eFluor-780 | e-fluor-780 | ThermoFisher | 65-0865-18 | 500 test | N/A | N/A |
| CD22 | | APC (sec. Ab) | | | | human | |
| Antigen | | | | | | | |
| mCherry | N/A | N/A | N/A | N/A | N/A | N/A | N/A |
| huCD45 | HBO | BUV395 | BD Horizon | 563792 | 100 test | human | mono |
| huCD3 | SK7 | BUV805 | BD Horizon | BDB612894 | 25 test | human | mono |
| huCD4 | SK3 | BV711 | BD Horizon | 344648 | 100 test | human | mono |
| huCD8 | RPA-T8 | BV421 | BD Horizon | 562428 | 100 test | human | mono |
| mucD45 | 30-F11 | BB700 | BD Horizon | 566439 | 25 ug | mouse | mono |
| huc019 | H1B19 | BUV737 | BD | 741829 | 100 test | human | mono |
| | | | Biosciences | | | | |

### Example 65. In Vitro Protein Expression (GFP) in Primary Human T-Cells of Lipids 10, 15, 16, 24, 26 and Comparator Lipid aCD8 Targeted LNPs (ALC-0315) Stored at 4° C. and after One Freeze-Thaw Cycle (Post −80° C. Storage)

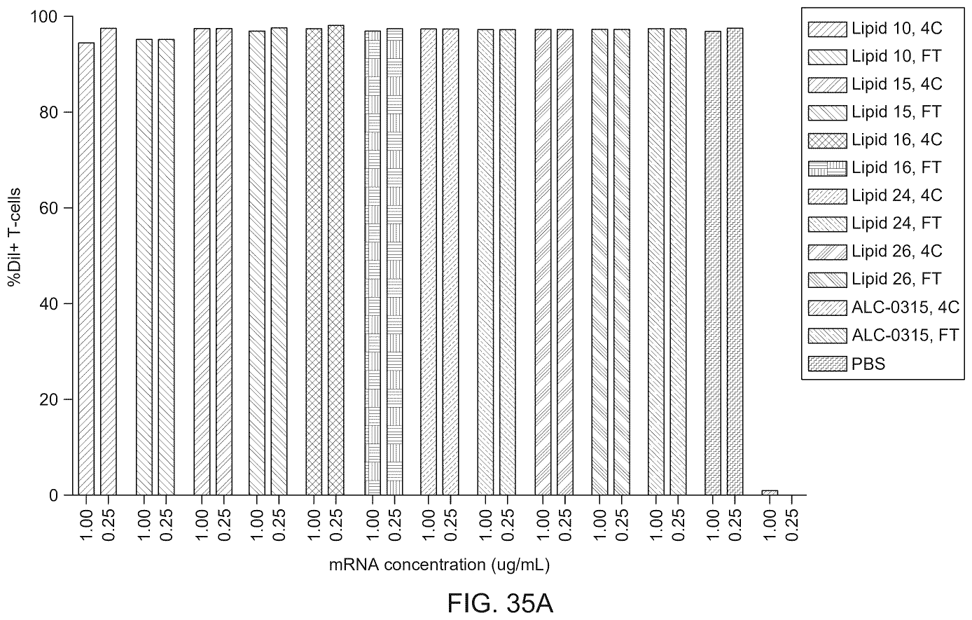

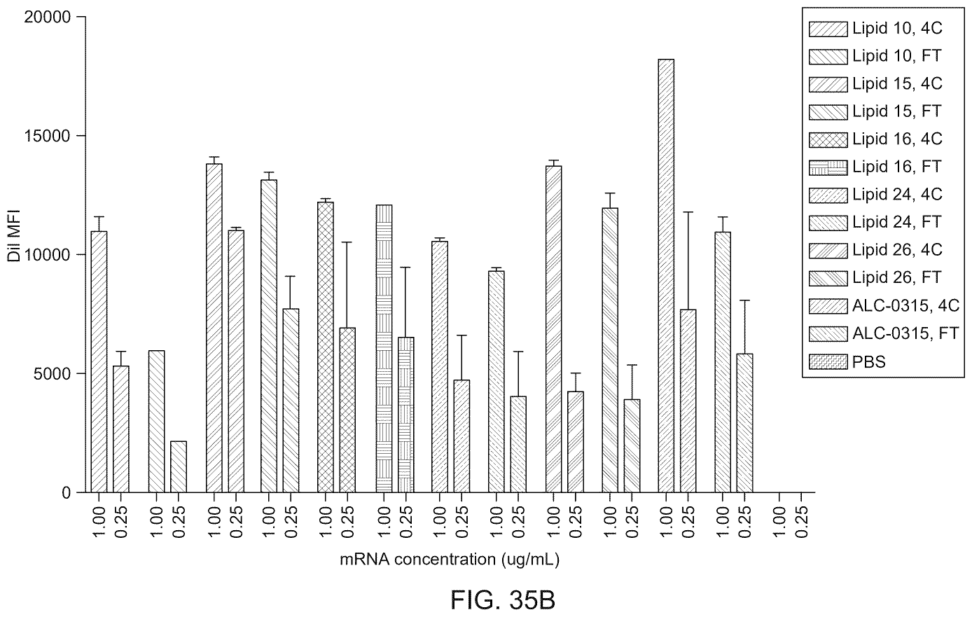

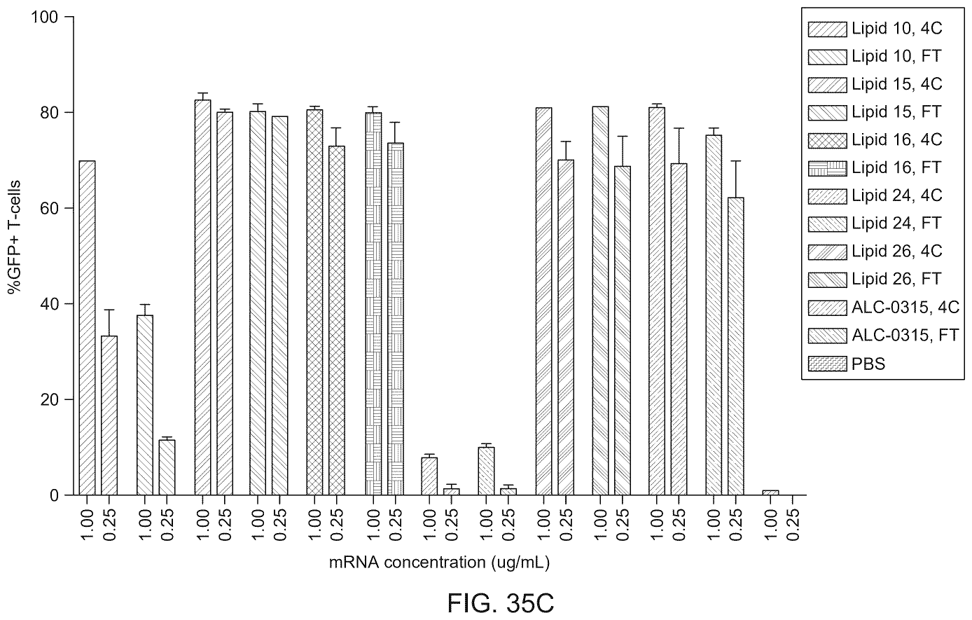

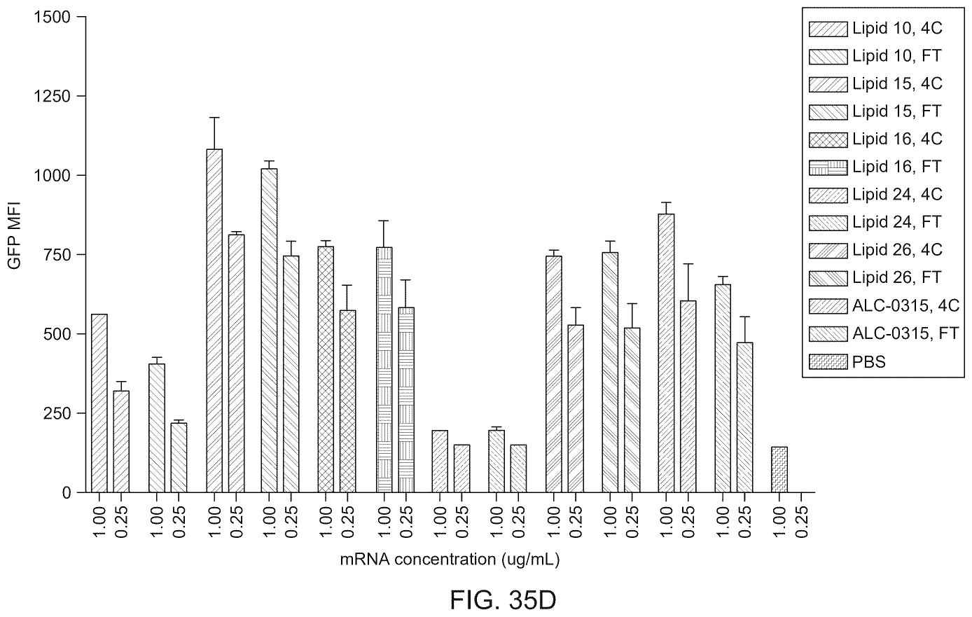

This example compares the GFP protein expression resulting from LNP's derived from Lipids 10, 15, 16, 24, and 26 to LNPs made using comparator lipids ALC-0315. Nanoparticles bearing GFP encoding mRNA (and optionally a fluorescent dye label (DiI-C18-5DS)) were produced using the microfluidic mixing and buffer exchange processes described in Example 45. An anti-CD8 VHH-conjugate was incorporated into the parent LNPs to obtain the final VHH-targeted LNP formulation using the process described in Example 48. Particles thus produced were tested in vitro in primary human CD8+ T cells to assess reporter RNA expression as described in Example 51. At 1 μg/mL and 0.25 μg/mL, a similar number of T-cells showed LNP association as determined by the % DiI+ T-cells (FIG. 35A). At the 1 μg/mL dose, DiI MFI values for ALC-0315 showed a drop from 1445.8 to 870.1 (FIG. 35B and Table 34) after one freeze-thaw cycle. This was not observed for Lipid 15 where the change in Dil MFI was less remarkable, 1096.3 to 1040.8 post 1 freeze-thaw cycle, indicating that LNPs based on Lipid 15 has improved freeze/thaw stability over ALC-0315 LNPs.

TABLE 34
DiI MFI values of Lipids 10, 15, 16, 24, and 26 and comparator lipid ALC-
0315 at mRNA doses 1 ug/mL and 0.25 ug/mL and fold over background

| | | | | | | | Fold over | Fold over |
|---|---|---|---|---|---|---|---|---|
| | | | | | | | background | background |
| DiI MFI | 1 ug/mL | | 0.25 ug/mL | | 0 ug/mL | | (1 ug/mL) | (0.25 ug/mL) |
| Lipid 10, 4C | 10594 | 11500 | 5806 | 4945 | | | 873.3 | 424.9 |
| Lipid 10, FT | 5940 | 6012 | 2250 | 2185 | | | 472.4 | 175.3 |
| Lipid 15, 4C | 14074 | 13663 | 10963 | 11222 | | | 1096.3 | 876.9 |
| Lipid 15, FT | 13447 | 12885 | 8775 | 6821 | | | 1040.8 | 616.4 |
| Lipid 16, 4C | 12258 | 12341 | 9529 | 4358 | | | 972.3 | 548.9 |
| Lipid 16, FT | 12086 | 12111 | 8716 | 4525 | | | 956.4 | 523.4 |
| Lipid 24, 4C | 10710 | 10675 | 6110 | 3449 | | | 845.3 | 377.8 |
| Lipid 24, FT | 9442 | 9357 | 5428 | 2838 | | | 743.0 | 326.7 |
| Lipid 26, 4C | 13513 | 13953 | 4873 | 3876 | | | 1085.6 | 345.8 |
| Lipid 26, FT | 12513 | 11600 | 5000 | 2970 | | | 953.1 | 315.0 |
| ALC-0315, 4C | 18332 | 18247 | 10661 | 4898 | | | 1445.8 | 615.0 |
| ALC-0315, FT | 11467 | 10546 | 7502 | 4188 | | | 870.1 | 462.1 |
| PBS | | | | | 12.1 | 13.2 | | |

At the 1 μg/mL dose, a similar number of CD8+ T-cells were transfected with Lipid 15 and ALC-0315 LNPs, however, at the 0.25 μg/mL dose, a larger fraction of T-cells were GFP+ with Lipid 15 LNPs (FIG. 35C) and the GFP MFI values were consistently increased at all doses suggesting more efficient cytosolic access/cytosolic release of encapsulated GFP mRNA relative to ALC-0315 LNPs (FIG. 35D). The performance levels and relative order of LNP efficiency were well preserved for Lipid 15 LNPs tested after 1 freeze-thaw cycle (Post −80° C. storage) as illustrated by the comparison of % GFP+ cells and GFP MFI values before and after −80° C. storage (FIG. 35C, FIG. 35D, and Table 35).

TABLE 35
GFP MFI values of Lipids 10, 15, 16, 24, and 26 and comparator lipid ALC-
0315 at mRNA doses 1 ug/mL and 0.25 ug/mL and fold over background

| | | | | | | | Fold over | Fold over |
|---|---|---|---|---|---|---|---|---|
| | | | | | | | background | background |
| GFP MFI | 1 ug/mL | | 0.25 ug/mL | | 0 ug/mL | | (1 ug/mL) | (0.25 ug/mL) |
| Lipid 10, 4C | 555 | 558 | 337 | 300 | | | 44.0 | 25.2 |
| Lipid 10, FT | 415 | 393 | 216 | 218 | | | 31.9 | 17.2 |
| Lipid 15, 4C | 1153 | 1003 | 811 | 799 | | | 85.2 | 63.6 |
| Lipid 15, FT | 1035 | 1014 | 777 | 718 | | | 81.0 | 59.1 |
| Lipid 16, 4C | 785 | 753 | 629 | 514 | | | 60.8 | 45.2 |
| Lipid 16, FT | 833 | 717 | 641 | 516 | | | 61.3 | 45.7 |
| Lipid 24, 4C | 193 | 187 | 150 | 141 | | | 15.0 | 11.5 |
| Lipid 24, FT | 204 | 192 | 152 | 142 | | | 15.7 | 11.6 |
| Lipid 26, 4C | 754 | 727 | 560 | 484 | | | 58.5 | 41.3 |
| Lipid 26, FT | 779 | 734 | 568 | 460 | | | 59.8 | 40.6 |
| ALC-0315, 4C | 901 | 842 | 684 | 503 | | | 68.9 | 46.9 |
| ALC-0315, FT | 670 | 625 | 524 | 414 | | | 51.2 | 37.1 |
| PBS | | | | | 133 | 136 | | |

### Example 66-1. Viability, LNP Association (Measured as DiI-Dye Fluorescence), and GFP Expression in Human CD8+ T-Cells Transfected with aCD8 (1705F, 2205F, 15C01 (SEQ ID 1), 1333F, 988F, TRX2) or Non-Binding (Mutated OKT8) Targeted DLn-KC2-DMA LNPs

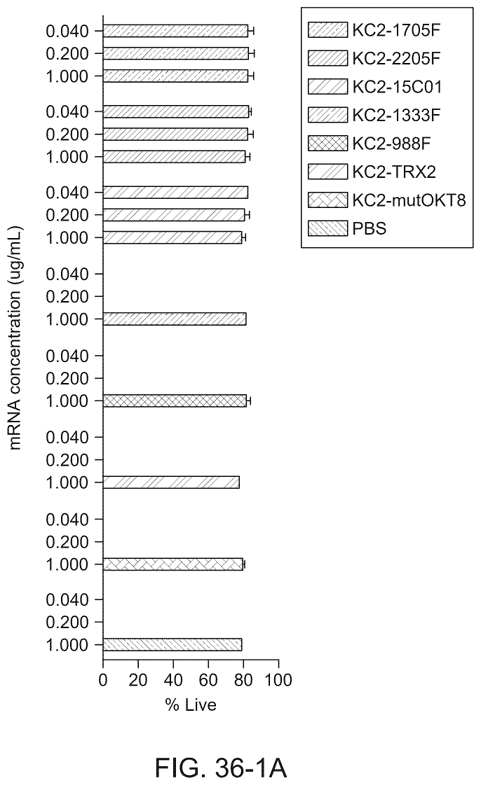

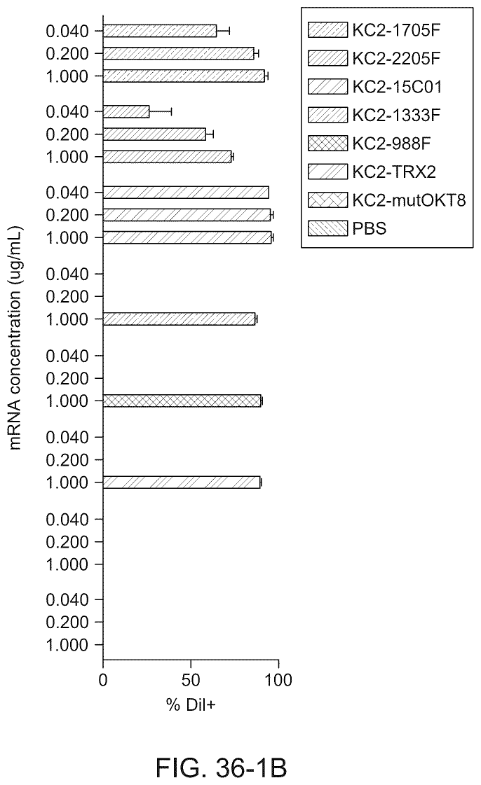

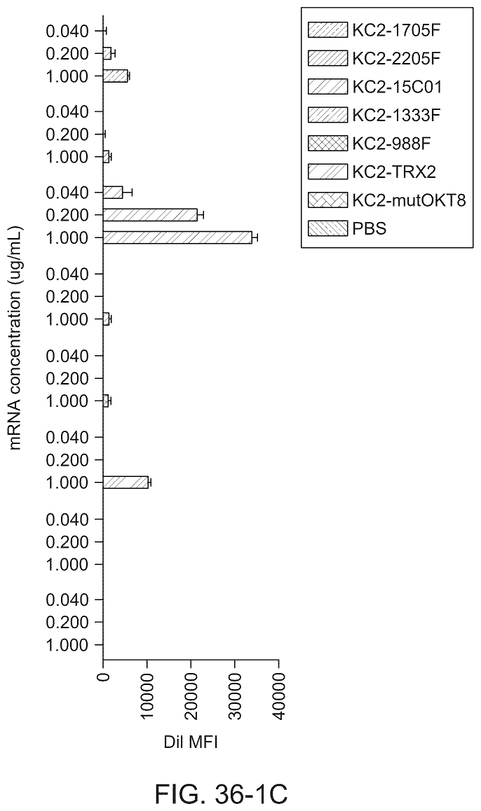

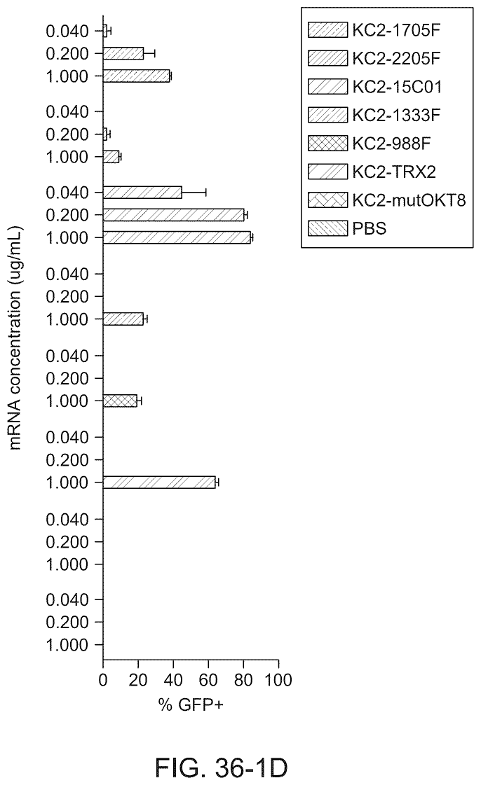

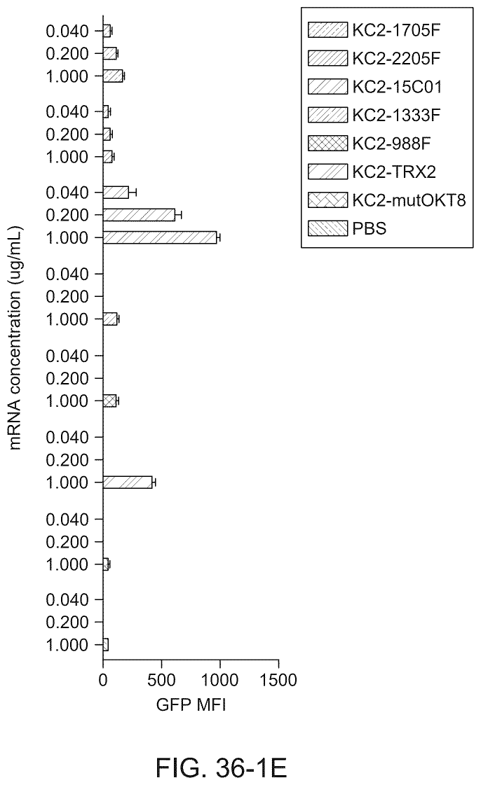

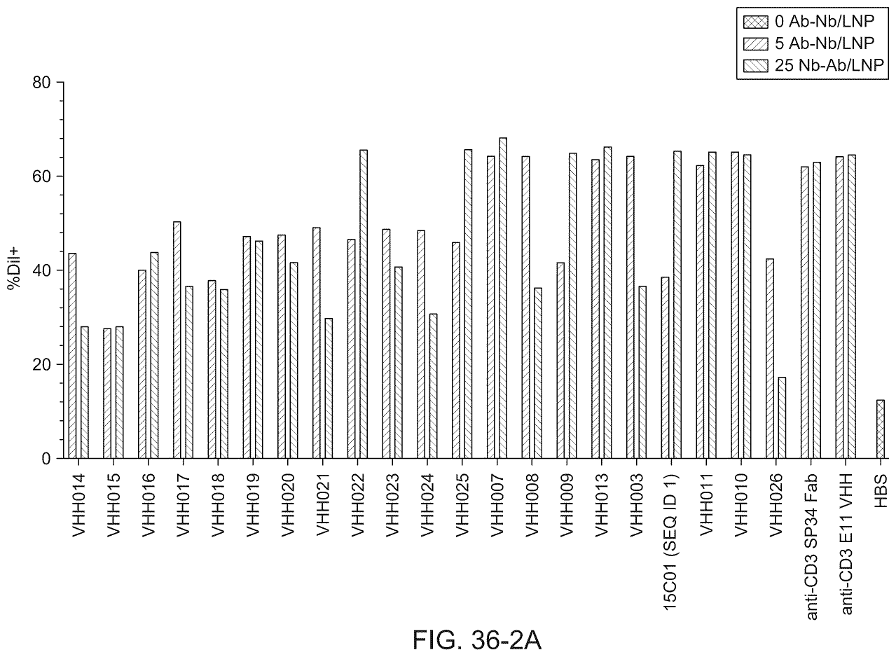

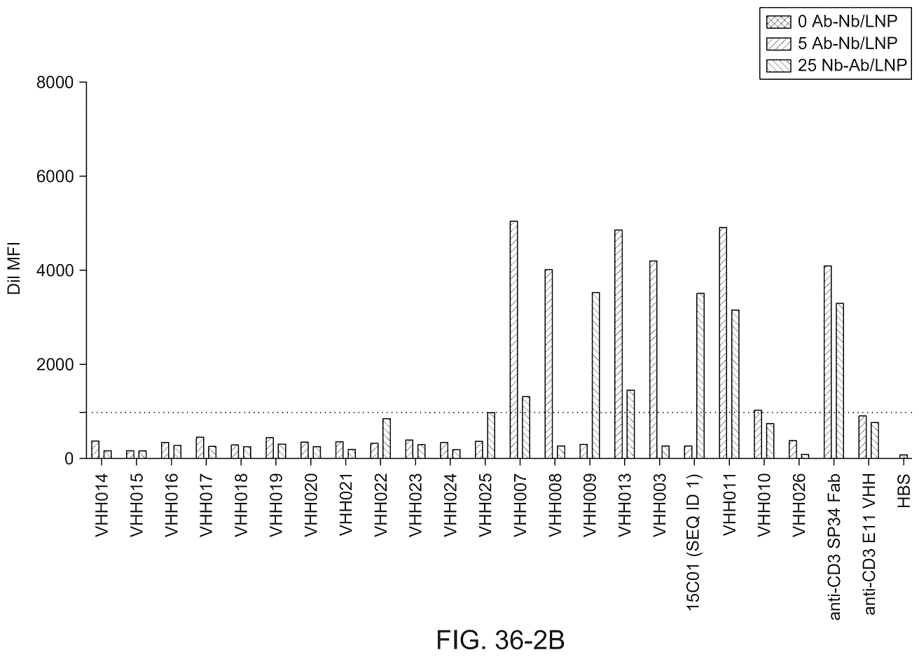

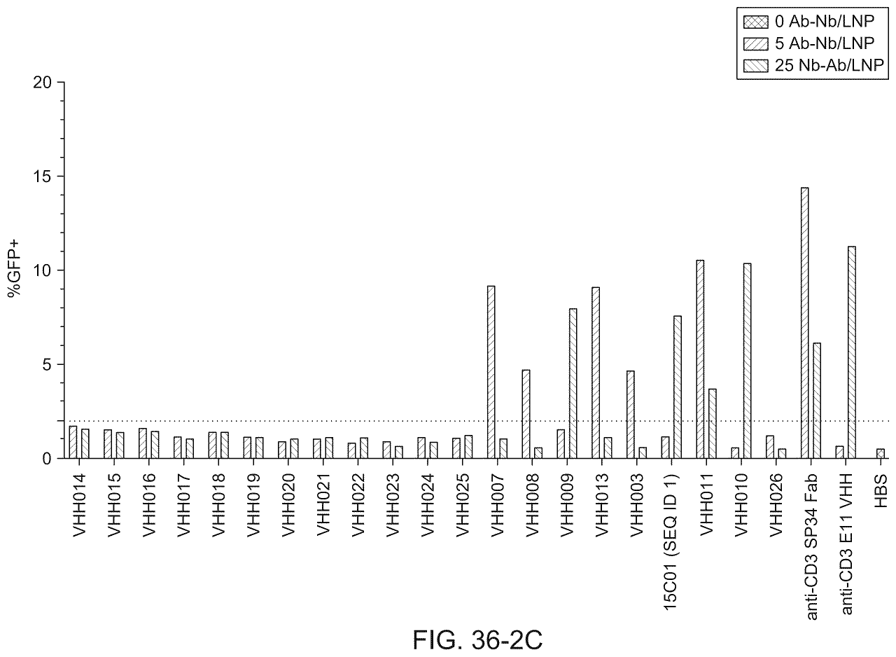

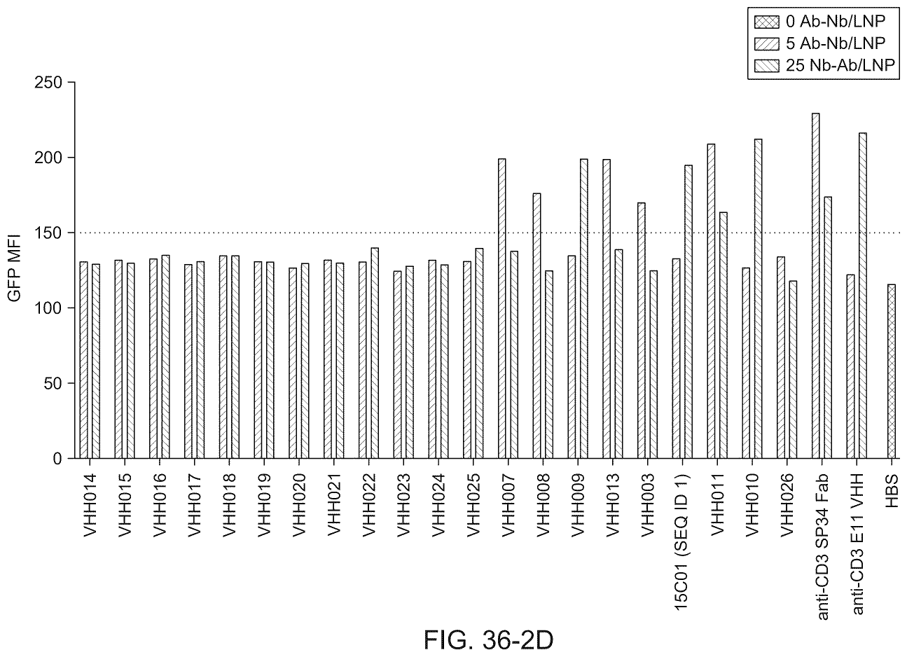

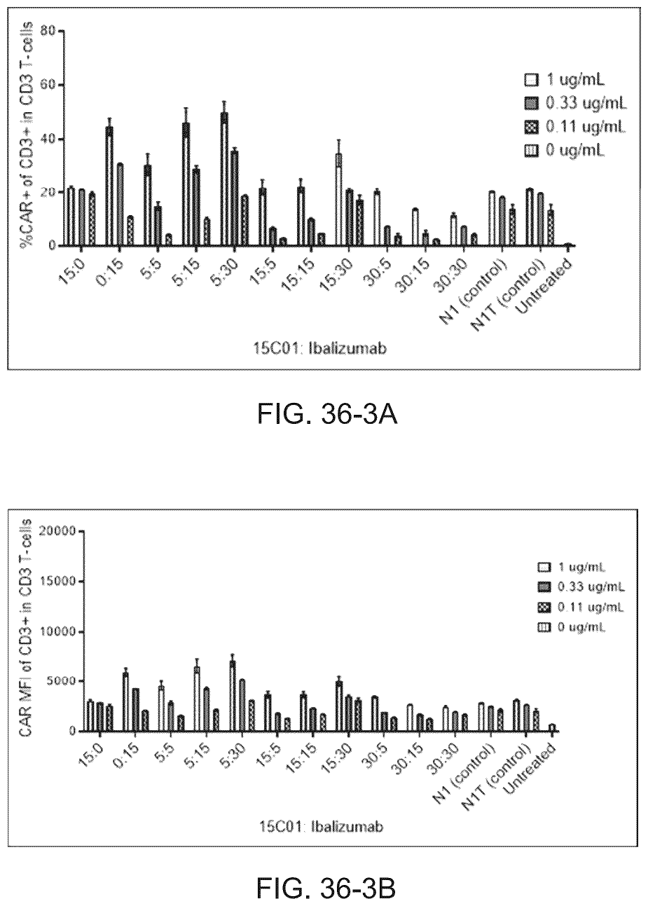

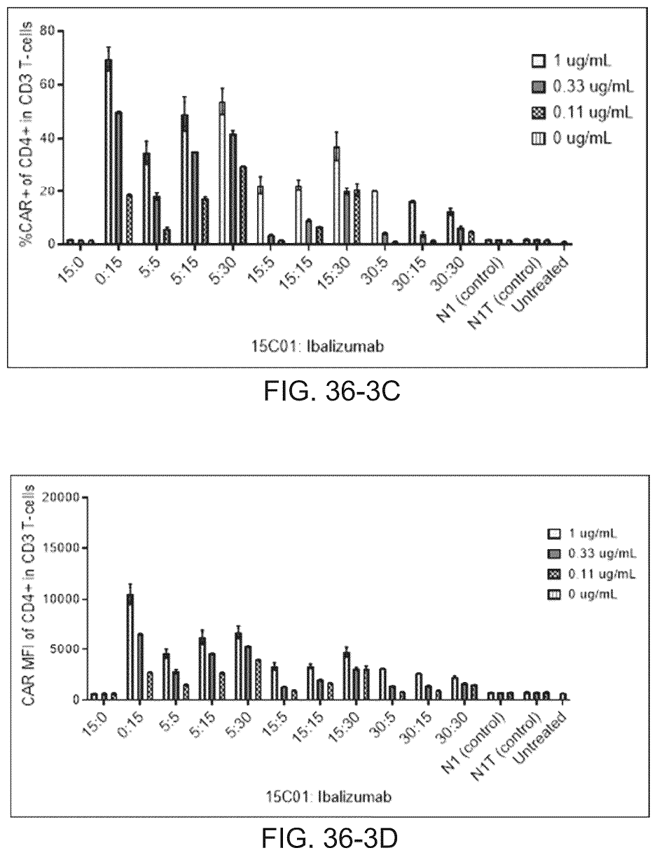

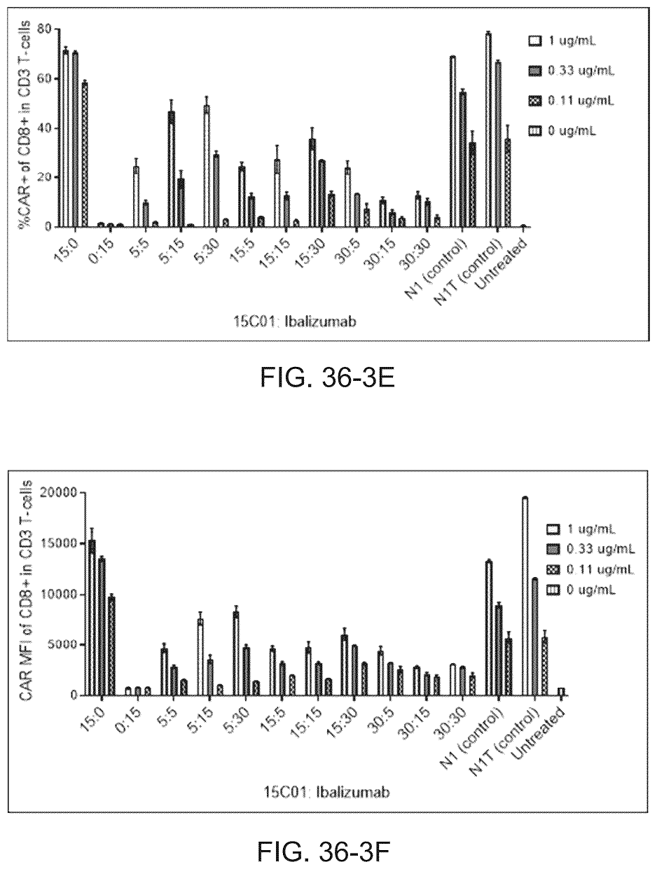

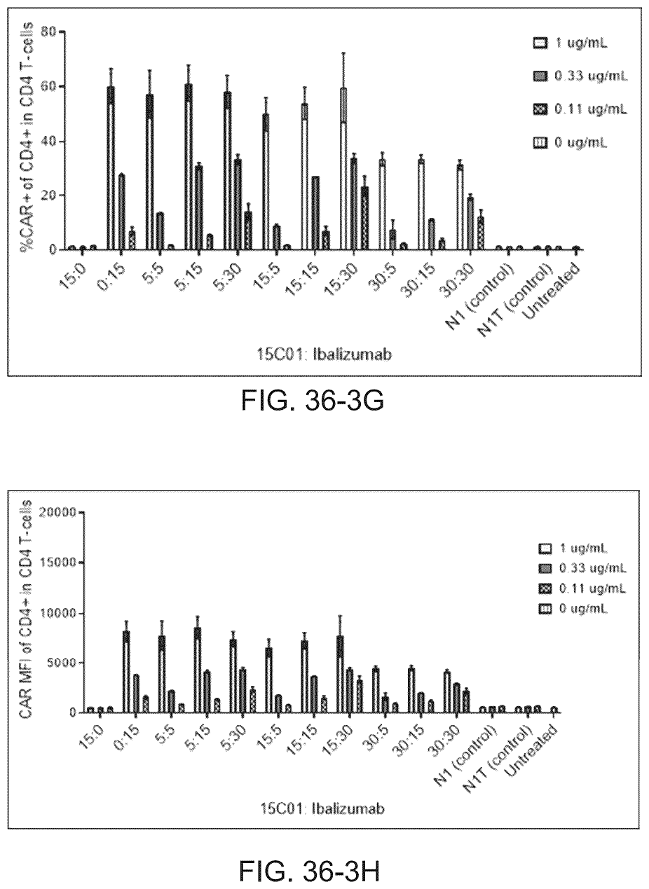

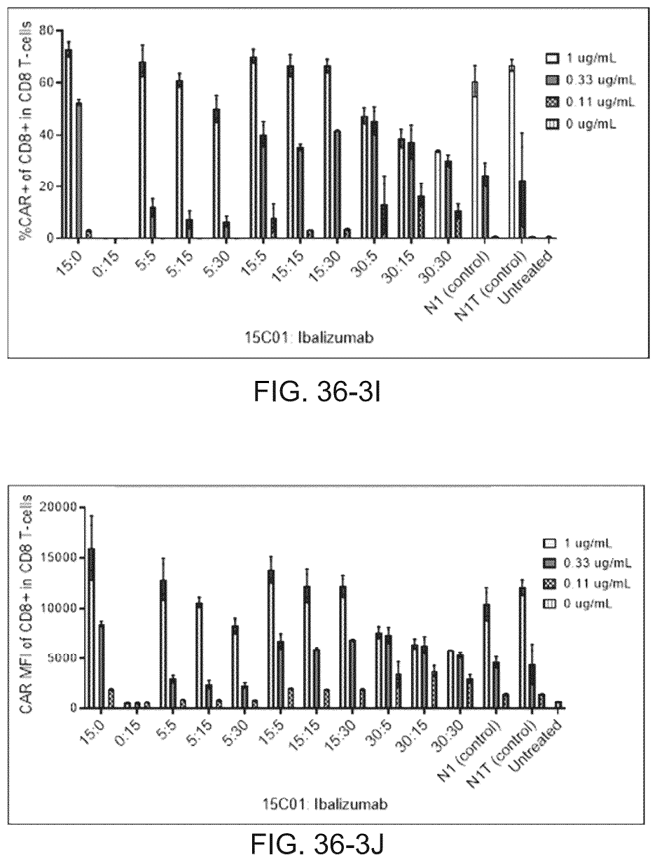

This example compares the viability, DiI association, and GFP protein expression resulting from DLn-KC2-DMA LNPs with CD8-targeting moieties 1705F, 2205F, 15C01 (SEQ ID 1), 1333F, 988F, and TRX2. Nanoparticles bearing GFP encoding mRNA (and a fluorescent dye label (DiI-C18-5DS)) were produced using the microfluidic mixing and buffer exchange processes described in Example 45. An αCD8 Fab- or VHH-conjugate was incorporated into the parent LNPs to obtain the final antibody targeted LNP formulation using the process described in Example 48. Particles thus produced were tested in vitro in primary human CD8+ T cells to assess reported gene expression as described in Example 51. All targeting moieties tested showed a similar level of T-cell viability to the untreated control 24 hours post-transfection (FIG. 36-1A). At all dose levels, 15C01 (SEQ ID 1) LNPs outperformed other tested targeting moieties both in terms of the fraction of DiI+ T-cells (reflected by % DiI+ cells) (FIG. 36-1B), the amount of LNP association per cell (reflected by DiI MFI values) (FIG. 36-1C), fraction of GFP+ T-cells (reflected by % GFP+ cells) (FIG. 36-1D), and the copies of GFP protein produced on a per cell basis (reflected by GFP MFI values) (FIG. 36-1E). 15C01 (SEQ ID 1) LNPs resulted in roughly 18-fold increased GFP MFI values over the untreated control with TRX2 LNPs resulting in an 8-fold increase in GFP MFI over the untreated control suggesting more efficient receptor-mediated endocytosis with 15C01 (SEQ ID 1) LNPs relative to TRX2 LNPs.

### Example 66-2. Viability, LNP Association (Measured as DiI-Dye Fluorescence), and GFP Expression in Human CD8+ T-Cells Transfected with ACD8 (VHH003, VHH007-VHH011, VHH013-VHH026, or 15C01 (SEQ ID 1)) or ACD3 (SP34 or 60E11) Targeted DLn-KC2-DMA LNPs

This example compares the viability, Dil association, and GFP protein expression resulting from DLn-KC2-DMA LNPs with CD8-targeting moieties VHH003, VHH007-VHH011, VHH013-VHH026, and 15C01 (SEQ ID 1). Nanoparticles bearing GFP encoding mRNA (and a fluorescent dye label (DiI-C18-5DS)) were produced using the microfluidic mixing and buffer exchange processes described in Example 45. An αCD8 VHH- or an αCD3 Fab- or VHH-conjugate was incorporated into the parent LNPs to obtain the final antibody-targeted LNP formulation using the process described in Example 48. Particles thus produced were tested in vitro in primary human CD8+ T cells to assess reported gene expression as described in Example 51. LNPs inserted with the αCD8 VHH-conjugates, VHH022, VHH025, VHH007, VHH008, VHH009, VHH013, VHH003, 15C01 (SEQ ID 1), VHH011, or VHH026 at densities 5 and/or 25 VHHs per LNP, showed increased cell-LNP association in terms of the fraction of DiI+ T-cells (reflected by % DiI+ cells) (FIG. 36-2A and Table 36-1). antiCD3 VHH 60E11 is described in International Patent Publication No. WO2016/180982.

TABLE 36-1
% DiI+ cells of antiCD8 (VHH003, VHH007-
VHH011, VHH013-VHH026, or 15C01 (SEQ ID 1)) or
antiCD3 (SP34 or 60E11) targeted DLN-KC2-DMA LNPs.

| % DiI+ | 0 Ab-Nb/LNP | | 5 Ab-Nb/LNP | | 25 Nb-Ab/LNP | |
|---|---|---|---|---|---|---|
| antiCD8 VHH014 | | | 31.4 | 55.9 | 30.4 | 25.4 |
| antiCD8 VHH015 | | | 24.2 | 31.1 | 28.5 | 28.6 |
| antiCD8 VHH016 | | | 34.2 | 46 | 41.7 | 46.3 |
| antiCD8 VHH017 | | | 36.2 | 64.7 | 35.7 | 37.6 |
| antiCD8 VHH018 | | | 36.5 | 39.3 | 40.2 | 31.8 |
| antiCD8 VHH019 | | | 41.6 | 52.9 | 47.6 | 45.1 |
| antiCD8 VHH020 | | | 38.5 | 56.9 | 45 | 38.3 |
| antiCD8 VHH021 | | | 56.8 | 41.5 | 31.9 | 27.8 |
| antiCD8 VHH022 | | | 46.2 | 46.7 | 67.8 | 63.3 |
| antiCD8 VHH023 | | | 39.4 | 58.3 | 41.5 | 40 |
| antiCD8 VHH024 | | | 53.5 | 43.6 | 33.2 | 28.2 |
| antiCD8 VHH025 | | | 44.4 | 47.6 | 68.3 | 63.2 |
| antiCD8 VHH007 | | | 64.6 | 64.1 | 70.2 | 66 |
| antiCD8 VHH008 | | | 64.3 | 64.1 | 38.5 | 34.1 |
| antiCD8 VHH009 | | | 36.5 | 47 | 66 | 64 |
| antiCD8 VHH013 | | | 62.7 | 64.6 | 67 | 65.6 |
| antiCD8 VHH003 | | | 64.4 | 64.3 | 37.5 | 35.4 |
| antiCD8 15C01 | | | 31.3 | 45.9 | 67.2 | 63.2 |
| (SEQ ID 1) | | | | | | |
| antiCD8 VHH011 | | | 61.9 | 62.8 | 64.3 | 66.2 |
| antiCD8 VHH010 | | | 65.2 | 65.8 | 72.6 | 56.7 |
| antiCD8 VHH026 | | | 40.5 | 44.1 | 33.9 | 0.37 |
| anti-CD3 SP34 Fab | | | 61.1 | 63 | 63.2 | 62.6 |
| anti-CD3 E11 VHH | | | 66 | 62.3 | 68.7 | 60.7 |
| HBS | 24.4 | 0.38 | | | | |

When evaluating the amount of LNP association per cell (reflected by the DiI MFI values) LNPs inserted with the αCD8 VHH-conjugates, VHH007, VHH008, VHH009, VHH013, VHH003, 15C01 (SEQ ID 1), VHH011, or VHH010 at densities 5 and/or 25 VHHs per LNP, showed to be superior to other αCD8 VHH-conjugates tested (FIG. 36-2B).

TABLE 36-2
DiI MFI values of antiCD8 (VHH003, VHH007-VHH011,
VHH013-VHH026, or 15C01 (SEQ ID 1)) or antiCD3
(SP34 or 60E11) targeted DLN-KC2-DMA LNPs.

| DiI MFI | 0 Ab-Nb/LNP | | 5 Ab-Nb/LNP | | 25 Nb-Ab/LNP | |
|---|---|---|---|---|---|---|
| antiCD8 VHH014 | | | 279 | 584 | 266 | 168 |
| antiCD8 VHH015 | | | 185 | 222 | 199 | 199 |
| antiCD8 VHH016 | | | 370 | 440 | 265 | 373 |
| antiCD8 VHH017 | | | 294 | 664 | 316 | 300 |
| antiCD8 VHH018 | | | 304 | 332 | 325 | 247 |
| antiCD8 VHH019 | | | 432 | 536 | 367 | 341 |
| antiCD8 VHH020 | | | 305 | 451 | 337 | 281 |
| antiCD8 VHH021 | | | 503 | 315 | 229 | 218 |
| antiCD8 VHH022 | | | 382 | 350 | 1040 | 741 |
| antiCD8 VHH023 | | | 332 | 499 | 337 | 316 |
| antiCD8 VHH024 | | | 464 | 355 | 268 | 218 |
| antiCD8 VHH025 | | | 434 | 359 | 1158 | 841 |
| antiCD8 VHH007 | | | 6083 | 4155 | 1192 | 1547 |
| antiCD8 VHH008 | | | 4897 | 3254 | 333 | 285 |
| antiCD8 VHH009 | | | 293 | 366 | 4834 | 2322 |
| antiCD8 VHH013 | | | 6041 | 3816 | 1419 | 1551 |
| antiCD8 VHH003 | | | 5044 | 3440 | 303 | 309 |
| antiCD8 15C01 | | | 233 | 379 | 4662 | 2448 |
| (SEQ ID 1) | | | | | | |
| antiCD8 VHH011 | | | 5893 | 3975 | 4489 | 1900 |
| antiCD8 VHH010 | | | 1069 | 1048 | 863 | 682 |
| antiCD8 VHH026 | | | 426 | 445 | 218 | 12.8 |
| anti-CD3 SP34 Fab | | | 4677 | 3612 | 4742 | 1913 |
| anti-CD3 E11 VHH | | | 1023 | 859 | 839 | 800 |
| HBS | 194 | 12.2 | | | | |

Similarly, LNPs inserted with the αCD8 VHH-conjugates, VHH007, VHH008, VHH009, VHH013, VHH003, 15C01 (SEQ ID 1), VHH011, or VHH010 at densities 5 and/or 25 VHHs per LNP, showed increased transfection efficiency both in terms of the fraction of GFP+ T-cells (reflected by % GFP+ cells) (FIG. 36-2C and Table 36-3) and in terms of the copies of GFP protein produced on a per cell basis (reflected by GFP MFI values) (FIG. 36-2D and Table 36-4).

TABLE 36-3
% GFP+ cells of antiCD8 (VHH003, VHH007-
VHH011, VHH013-VHH026, or 15C01 (SEQ ID 1)) or
antiCD3 (SP34 or 60E11) targeted DLN-KC2-DMA LNPs.

| % GFP+ | 0 Ab-Nb/LNP | | 5 Ab-Nb/LNP | | 25 Nb-Ab/LNP | |
|---|---|---|---|---|---|---|
| antiCD8 VHH014 | | | 1.79 | 1.72 | 1.67 | 1.48 |
| antiCD8 VHH015 | | | 1.36 | 1.67 | 1.13 | 1.6 |
| antiCD8 VHH016 | | | 1.8 | 1.57 | 1.4 | 1.57 |
| antiCD8 VHH017 | | | 1.25 | 1.15 | 1.1 | 1.13 |
| antiCD8 VHH018 | | | 1.27 | 1.59 | 1.42 | 1.38 |
| antiCD8 VHH019 | | | 1.3 | 1.12 | 1.04 | 1.24 |
| antiCD8 VHH020 | | | 0.99 | 0.87 | 1.14 | 1 |
| antiCD8 VHH021 | | | 1.35 | 0.81 | 0.83 | 1.42 |
| antiCD8 VHH022 | | | 0.86 | 0.83 | 0.95 | 1.46 |
| antiCD8 VHH023 | | | 0.87 | 0.91 | 0.71 | 0.68 |
| antiCD8 VHH024 | | | 1.46 | 0.8 | 0.63 | 1.13 |
| antiCD8 VHH025 | | | 1.11 | 1.09 | 1.41 | 1.11 |
| antiCD8 VHH007 | | | 13.6 | 4.92 | 0.6 | 1.62 |
| antiCD8 VHH008 | | | 4.58 | 5.07 | 0.47 | 0.75 |
| antiCD8 VHH009 | | | 0.84 | 2.26 | 10.2 | 5.82 |
| antiCD8 VHH013 | | | 14.3 | 3.97 | 0.74 | 1.63 |
| antiCD8 VHH003 | | | 4.15 | 5.21 | 0.51 | 0.74 |
| antiCD8 15C01 | | | 0.48 | 1.85 | 9.11 | 6.04 |
| (SEQ ID 1) | | | | | | |
| antiCD8 VHH011 | | | 11.9 | 9.29 | 6.04 | 1.39 |
| antiCD8 VHH010 | | | 0.65 | 0.53 | 13.1 | 7.76 |
| antiCD8 VHH026 | | | 0.74 | 1.77 | 0.63 | 0.37 |
| anti-CD3 SP34 Fab | | | 17.4 | 11.6 | 10.4 | 1.99 |
| anti-CD3 E11 VHH | | | 0.74 | 0.56 | 12.1 | 10.5 |
| HBS | 0.57 | 0.45 | | | | |

TABLE 36-4
GFP MFI values of antiCD8 (VHH003, VHH007-VHH011,
VHH013-VHH026, or 15C01 (SEQ ID 1)) or antiCD3
(SP34 or 60E11) targeted DLN-KC2-DMA LNPs.

| GFP MFI | 0 Ab-Nb/LNP | | 5 Ab-Nb/LNP | | 25 Nb-Ab/LNP | |
|---|---|---|---|---|---|---|
| antiCD8 VHH014 | | | 129 | 135 | 130 | 131 |
| antiCD8 VHH015 | | | 130 | 137 | 128 | 134 |
| antiCD8 VHH016 | | | 132 | 136 | 135 | 138 |
| antiCD8 VHH017 | | | 126 | 134 | 131 | 134 |
| antiCD8 VHH018 | | | 133 | 139 | 137 | 135 |
| antiCD8 VHH019 | | | 133 | 132 | 135 | 130 |
| antiCD8 VHH020 | | | 129 | 128 | 132 | 131 |
| antiCD8 VHH021 | | | 137 | 130 | 128 | 134 |
| antiCD8 VHH022 | | | 132 | 132 | 143 | 139 |
| antiCD8 VHH023 | | | 124 | 128 | 131 | 127 |
| antiCD8 VHH024 | | | 136 | 131 | 128 | 133 |
| antiCD8 VHH025 | | | 132 | 132 | 144 | 138 |
| antiCD8 VHH007 | | | 225 | 177 | 132 | 147 |
| antiCD8 VHH008 | | | 179 | 177 | 124 | 128 |
| antiCD8 VHH009 | | | 127 | 145 | 212 | 189 |
| antiCD8 VHH013 | | | 230 | 171 | 134 | 146 |
| antiCD8 VHH003 | | | 169 | 174 | 126 | 126 |
| antiCD8 15C01 | | | 125 | 144 | 207 | 187 |
| (SEQ ID 1) | | | | | | |
| antiCD8 VHH011 | | | 219 | 202 | 185 | 147 |
| antiCD8 VHH010 | | | 130 | 127 | 235 | 193 |
| antiCD8 VHH026 | | | 131 | 140 | 125 | 114 |
| anti-CD3 SP34 Fab | | | 253 | 208 | 207 | 145 |
| anti-CD3 E11 VHH | | | 128 | 118 | 229 | 207 |
| HBS | 123 | 112 | | | | |

### Example 66-3. CD22 CAR Expression in Human CD3+, CD4+, and CD8⁺ T-Cells Transfected with ACD8 (15C01V8) and ACD4 (Ibalizumab) Dual Targeted Lipid 15 LNPs Inserted at Various Densities

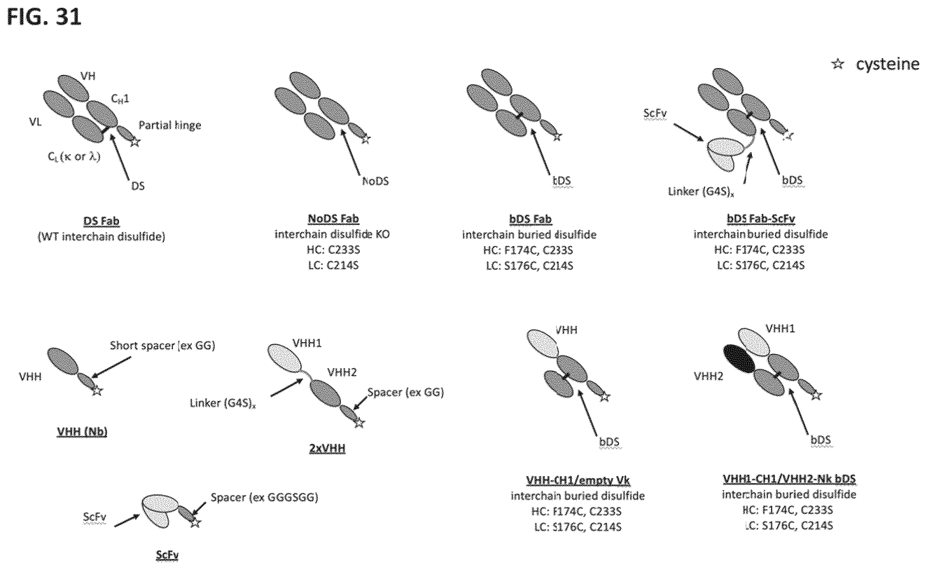

This example compares the CAR protein expression resulting from Lipid 15 LNPs dual inserted with CD8-(15C01v8 (SEQ ID 9)) and CD4− (Ibalizumab) targeting moieties. Nanoparticles bearing CD22 CAR (MRT14577 (SEQ ID 147)) encoding mRNA were produced using the microfluidic mixing and buffer exchange processes described in Example 45. An αCD8 VHH− and an αCD4 Fab-conjugate were incorporated into the parent LNPs at various densities (Table 36-5) to obtain the final antibody-targeted LNP formulation using the process described in Example 48. Particles thus produced were tested in vitro in primary human CD3+, CD4+, and CD8+ T cells to assess CAR expression as described in Example 51. The level of CD22 CAR expression was assessed using the method described in Example 54. LNPs inserted with 5 αCD8 VHH-conjugates (15C01v8) and 30 αCD4 Fab-conjugates (Ibalizumab) per LNP showed increased CAR expression both in terms of the fraction of CAR+CD3+ T-cells (reflected by % CAR+ of CD3+ in CD3 T-cells) and in terms of the CAR MFI values (reflected by CAR MFI of CD3+ in CD3 T-cells) (FIG. 36-3A and FIG. 36-3B). Similarly, when evaluating the CD4+ and CD8+ cells subsets separately, that could be identified by staining using an anti-CD4 and an anti-CD8 antibody, LNPs inserted with 5 αCD8 VHH-conjugates (15C01v8) and 30 αCD4 Fab-conjugates (Ibalizumab) per LNP showed increased CAR expression over other tested dual inserted densities both in terms of the fraction of CAR+CD4+ and CD8+ T-cells (reflected by % CAR+ of CD4+ and CD8+ in CD3 T-cells) and in terms of the CAR MFI values (reflected by CAR MFI of CD4+ and CD8+ in CD3 T-cells) (FIG. 36-3C-FIG. 36-3F). Finally, no change in trend could be observed in isolated CD4+ T-cells and isolated CD8+ T-cells, respectively, between LNPs inserted with the various densities of dual conjugate (FIG. 36-3G-FIG. 36-3J).

TABLE 36-5
Insertion densities of 15C01v8- and Ibalizumab-conjugates, respectively,
in Lipid 15 LNPs encapsulating CD22 CAR-encoding mRNA.

| | | | 15C01v8 | Ibalizumab |
|---|---|---|---|---|
| | | | Insertion | Fab Insertion |
| | | Ionizable | Density | Density |
| Sample ID | mRNA | Lipid | (Nb/LNP) | (Fab/LNP) |
| EXP2323027025-N1500 | TTR-102(SEQ ID 147) | Lipid 15 | 15 | 0 |
| EXP2323027025-N0015 | TTR-102(SEQ ID 147) | Lipid 15 | 0 | 15 |
| EXP2323027025-N0505 | TTR-102 (SEQ ID 147) | Lipid 15 | 5 | 5 |
| EXP2323027025-N0515 | TTR-102 (SEQ ID 147) | Lipid 15 | 5 | 15 |
| EXP2323027025-N0530 | TTR-102 (SEQ ID 147) | Lipid 15 | 5 | 30 |
| EXP2323027025-N1505 | TTR-102 (SEQ ID 147) | Lipid 15 | 15 | 5 |
| EXP2323027025-N1515 | TTR-102 (SEQ ID 147) | Lipid 15 | 15 | 15 |
| EXP2323027025-N1530 | TTR-102 (SEQ ID 147) | Lipid 15 | 15 | 30 |
| EXP2323027025-N3005 | TTR-102 (SEQ ID 147) | Lipid 15 | 30 | 5 |
| EXP2323027025-N3015 | TTR-102 (SEQ ID 147) | Lipid 15 | 30 | 15 |
| EXP2323027025-N3030 | TTR-102 (SEQ ID 147) | Lipid 15 | 30 | 30 |
| EXP2323027025-N1 | TTR-102 (SEQ ID 147) | Lipid 15 | 15 | 0 |
| EXP23011097-N1T | TTR-102 (SEQ ID 147) | Lipid 15 | 15 | 0 |

### Example 67. In Vitro LNP Association (Measured as DiI-Dye Fluorescence) and Protein Expression (GFP) in Primary Human CD8+ T-Cells Transfected with Lipid 15 LNPs Inserted with ACD8 (15C01V8 or TRX2), ACD3 (SP34), or Non-Binding (Mutated OKT8) Targeting Moieties

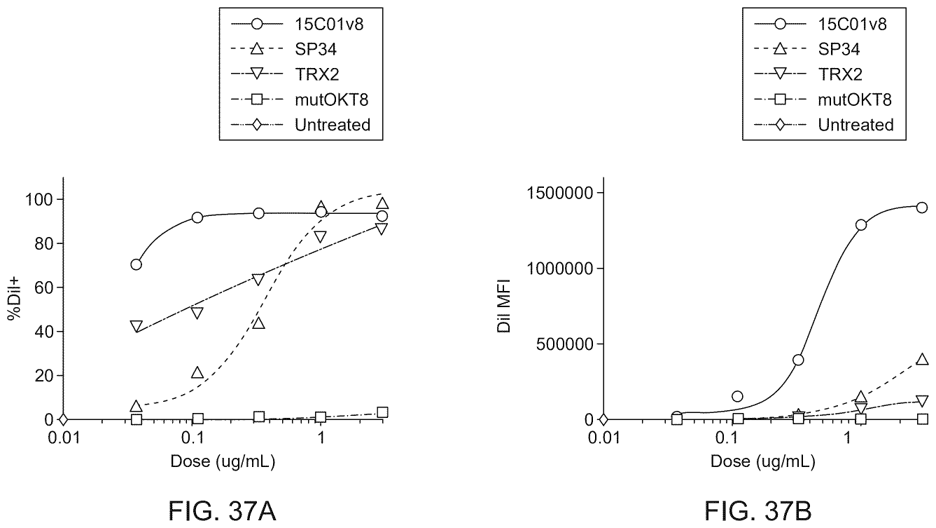

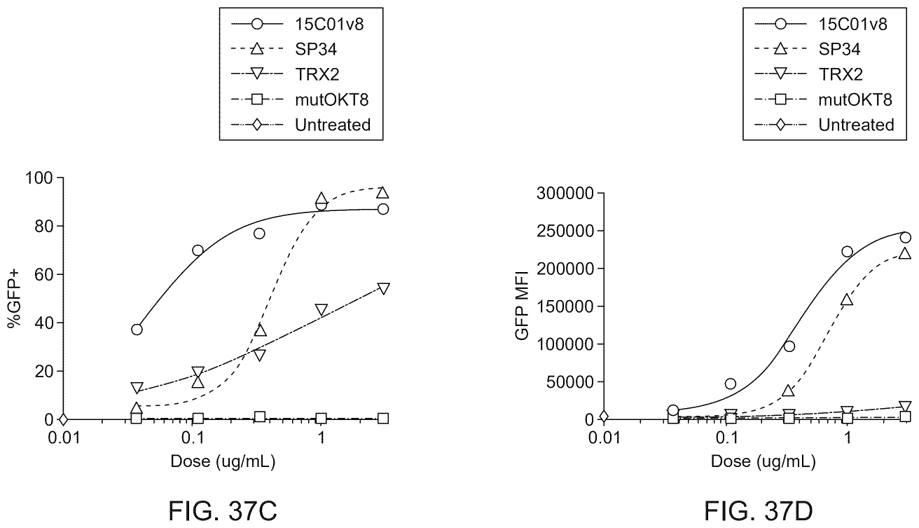

This example compares Dil association and GFP protein expression resulting from Lipid 15 LNPs with CD8-targeting moieties (15C01v8 or TRX2) and CD3-targeting moieties (SP34) at various dose levels. Nanoparticles bearing GFP encoding mRNA (and a fluorescent dye label (DiI-C18-5DS)) were produced using the microfluidic mixing and buffer exchange processes described in Example 45. An αCD8 Fab- or VHH-conjugate or αCD3 Fab was incorporated into the parent LNPs to obtain the final antibody targeted LNP formulation using the process described in Example 48. Particles thus produced were tested in vitro in primary human CD8+ T cells to assess LNP association and reporter protein expression as described in Example 51. At dose levels<1 μg/mL, 15C01v8 LNPs outperformed other tested targeting moieties in terms of the fraction of DiI+ T-cells (reflected by % DiI+ cells) (FIG. 37A). 15C01v8 was superior to other tested targeting moieties at all dose levels in terms of DiI MFI (FIG. 37B and Table 37).

TABLE 37
DiI MFI fold over untreated background values of
antiCD3- and antiCD8-targeted Lipid 15 LNPs at
mRNA doses 3, 1, 0.33, 0.11, and 0.037 ug/mL.

| | DiI MFI (Fold over background) | | | | |
|---|---|---|---|---|---|
| | 3 | 1 | 0.33 | 0.11 | 0.037 |
| | ug/mL | ug/mL | ug/mL | ug/mL | ug/mL |
| 15C01v8 | 10714.6 | 9819.6 | 3026.3 | 1151.4 | 114.6 |
| mutOKT8 | 5.6 | 3.0 | 2.0 | 1.1 | 1.0 |
| SP34 | 3037.3 | 1146.8 | 280.5 | 27.5 | 7.6 |
| TRX2 | 909.5 | 493.5 | 111.5 | 55.7 | 37.5 |

Similarly, at dose levels<1 μg/mL, 15C01v8 LNPs outperformed other tested targeting moieties in terms of the fraction of GFP+ T-cells (reflected by % GFP+ cells) (FIG. 37C). 15C01v8 was superior to other tested targeting moieties at all dose levels in terms of GFP MFI (FIG. 37D and Table 38).

TABLE 38
GFP MFI fold over untreated background values of
antiCD3- and antiCD8-targeted Lipid 15 LNPs at
mRNA doses 3, 1, 0.33, 0.11, and 0.037 ug/mL.

| | GFP MFI (Fold over background) | | | | |
|---|---|---|---|---|---|
| | 3 | 1 | 0.33 | 0.11 | 0.037 |
| | ug/mL | ug/mL | ug/mL | ug/mL | ug/mL |
| 15C01v8 | 58.8 | 53.7 | 23.5 | 11.5 | 2.4 |
| mutOKT8 | 1.0 | 0.7 | 0.7 | 0.7 | 0.7 |
| SP34 | 53.0 | 38.5 | 9.3 | 1.6 | 1.1 |
| TRX2 | 4.1 | 2.3 | 1.4 | 1.3 | 1.2 |

### Example 68. In Vitro LNP Association (Measured as DiI-Dye Fluorescence) and Protein Expression (GFP) in Primary Human CD8+ T-Cells Transfected with Lipid 15 LNPs Inserted with aCD8 (15C01V8 or TRX2), ACD3 (SP34), or Non-Binding (Mutated OKT8) Targeting Moieties at Various Time Points

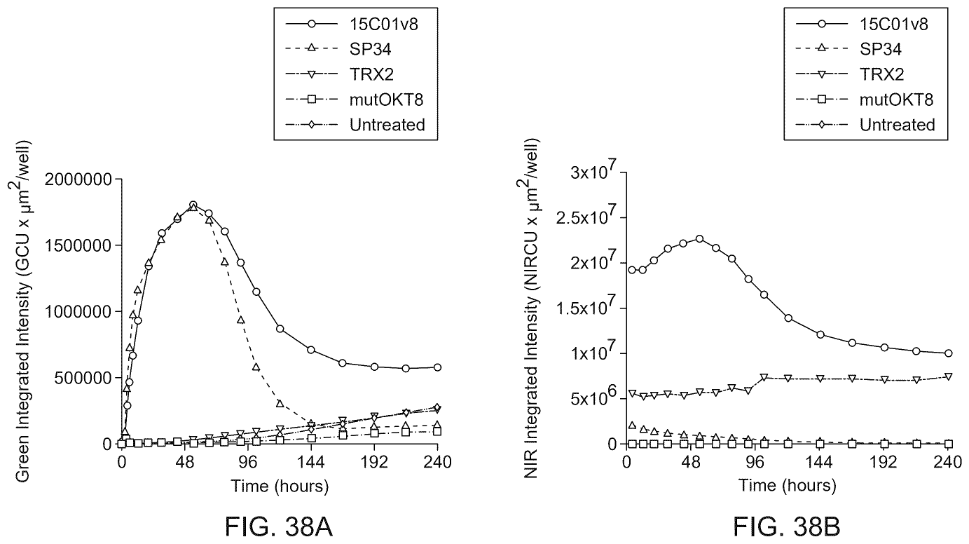

This example compares Dil association and GFP protein expression resulting from Lipid 15 LNPs with CD8-targeting moieties (15C01v8 or TRX2) and CD3-targeting moieties (SP34) at various time points. Nanoparticles bearing GFP encoding mRNA (and a fluorescent dye label (DiI-C18-5DS)) were produced using the microfluidic mixing and buffer exchange processes described in Example 45. An αCD8 Fab- or VHH-conjugate or αCD3 Fab was incorporated into the parent LNPs to obtain the final antibody targeted LNP formulation using the process described in Example 48. Particles thus produced were tested in vitro in primary human CD8+ T cells to assess LNP association and reporter protein expression as described in Example 51, but with the Dil signal and GPF expression being assessed at the various time points by utilizing the live cell imaging SX5 Incucyte system. Cells treated with 15C01v8- and SP34-targeted LNPs reached a maximum in GFP expression around the same time point (~50 hours) as depicted by Green Integrated Intensity. However, cells treated with 15C01v8-targeted LNPs showed increased durability of GFP expression compared to cells treated with SP34-targeted LNPs during the time course tested (up to 240 hours) indicating that 15C01v8-targeted LNPs can deliver the mRNA payload to CD8+ T-cells over extended time periods (FIG. 38A). Cells treated with 15C01v8-targeted LNPs showed increased Dil signal compared to SP34- and TRX2-targeted LNPs at all time points tested as depicted by NIR Integrated Intensity (FIG. 38B) indicating increased LNP association of 15C01v8-targeted LNPs compared to other targeting moieties tested.

### Example 69. LNP Association (Measured as DiI-Dye Fluorescence) and GFP Expression in Human CD8+ T-Cells Transfected with KC3 LNPs Containing Different Densities of aCD8 (15C01 and TRX2) Targeting Moieties

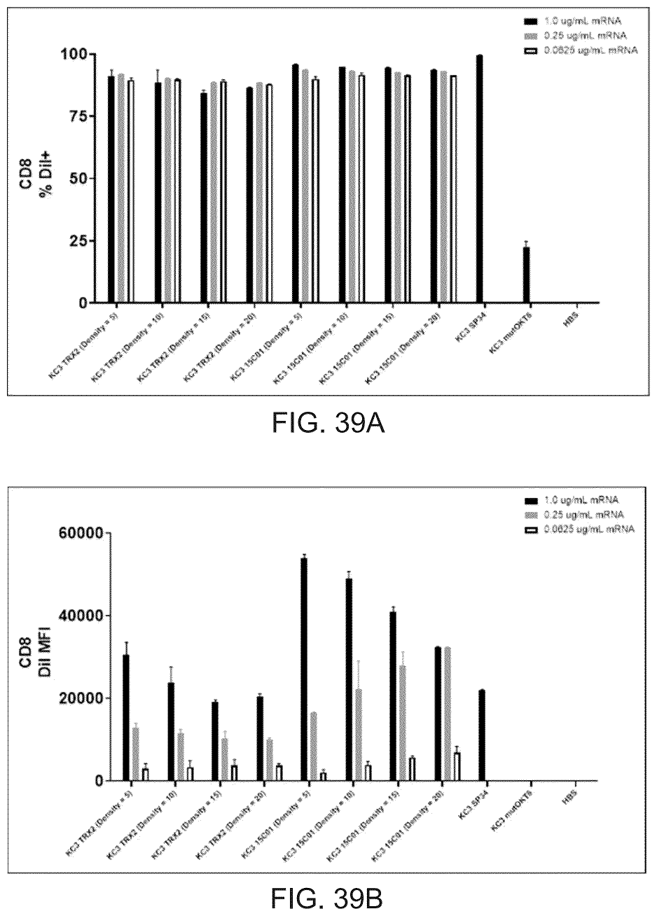

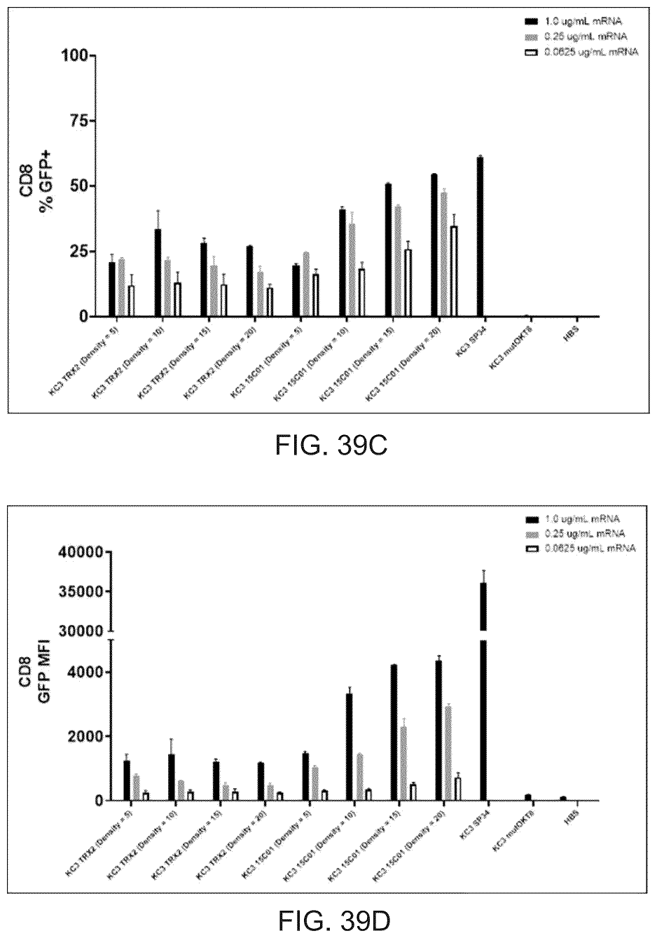

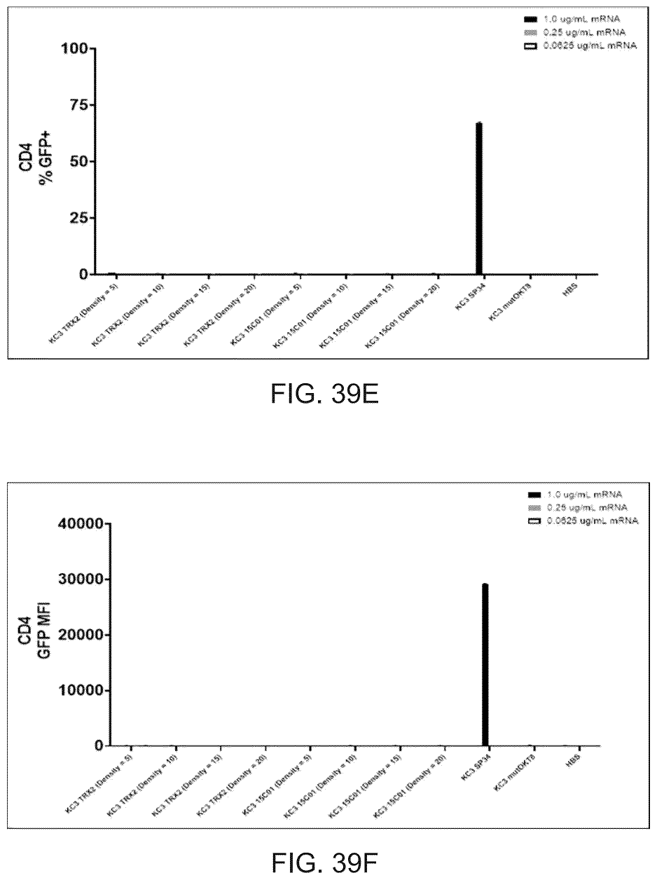

This example compares Dil association and GFP protein expression resulting from KC3 LNPs with different densities of CD8-targeting moieties (15C01 and TRX2) at different dose levels. Nanoparticles bearing GFP encoding mRNA (and a fluorescent dye label (DiI-C18-5DS)) were produced using the microfluidic mixing and buffer exchange processes described in Example 45. An αCD8 Fab- or VHH-conjugate or αCD3 Fab was incorporated into the parent LNPs to obtain the final antibody targeted LNP formulation using the process described in Example 48. Particles thus produced were tested in vitro in primary human CD3+ T cells to assess LNP association and reporter protein expression as described in Example 51. At all dose levels, LNPs performed similar between the various targeting moiety densities tested in terms of the fraction of DiI+ T-cells (reflected by % DiI+ cells) (FIG. 39A). At the 1 μg/mL dose, DiI MFI values decreased with the increasing 15C01 densities from 5 VHHs per LNP to 20 VHHs per LNP. However, at dose levels<1 g/mL DiI MFI values increased with the increasing 15C01 densities (FIG. 39B). At all dose levels, transfection efficiency was increased with the increasing densities of 15C01 in terms of the fraction of GFP+ T-cells (reflected by % GFP+ cells) (FIG. 39C). A similar trend was observed in terms of GFP MFI values (FIG. 39D). Expectedly, no GFP expression was observed in CD4+ T-cells with CD8-targeted LNPs (15C01 and TRX2) both in terms of % GFP+ and GFP MFI indicating targeting specificity toward CD8+ cells (FIG. 39E and FIG. 39F).

### Example 70. Viability, LNP Association (Measured as DiI-Dye Fluorescence), and GFP Expression in Human CD8+ T-Cells Transfected with Lipid 15 LNPs Containing Different Variants of 15C01 (SEQ ID 1, SEQ ID 7, SEQ ID 8, SEQ ID 9)

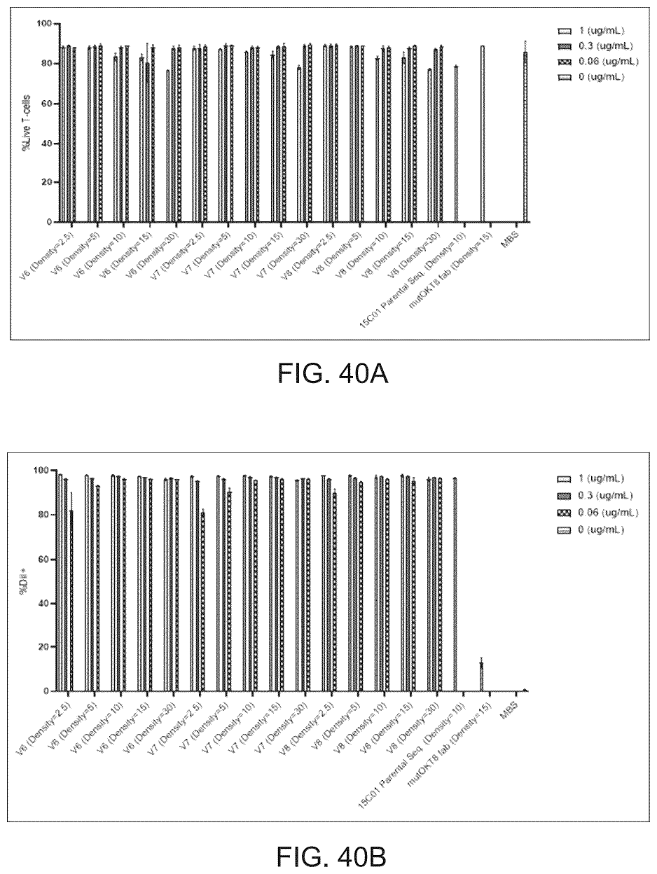

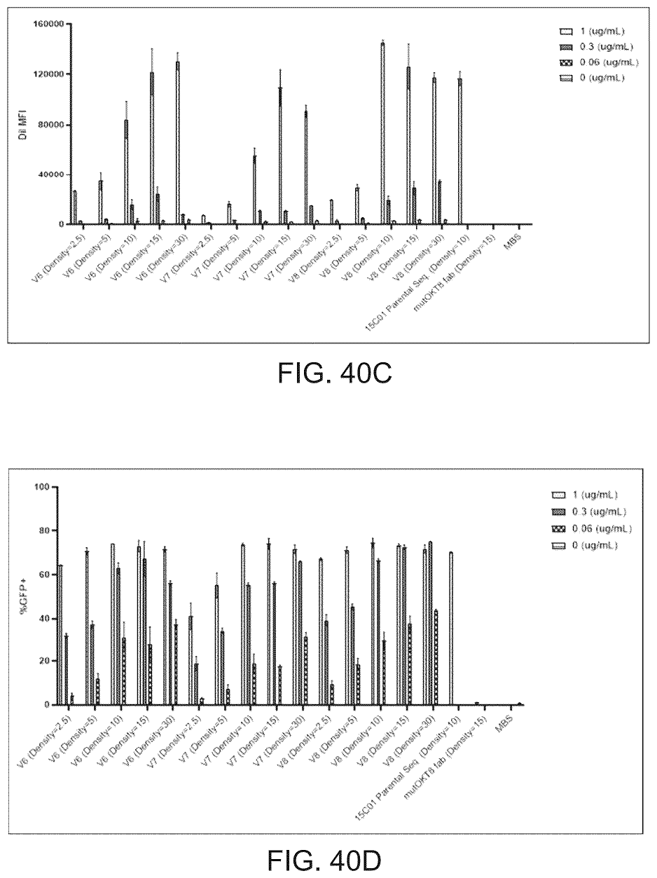

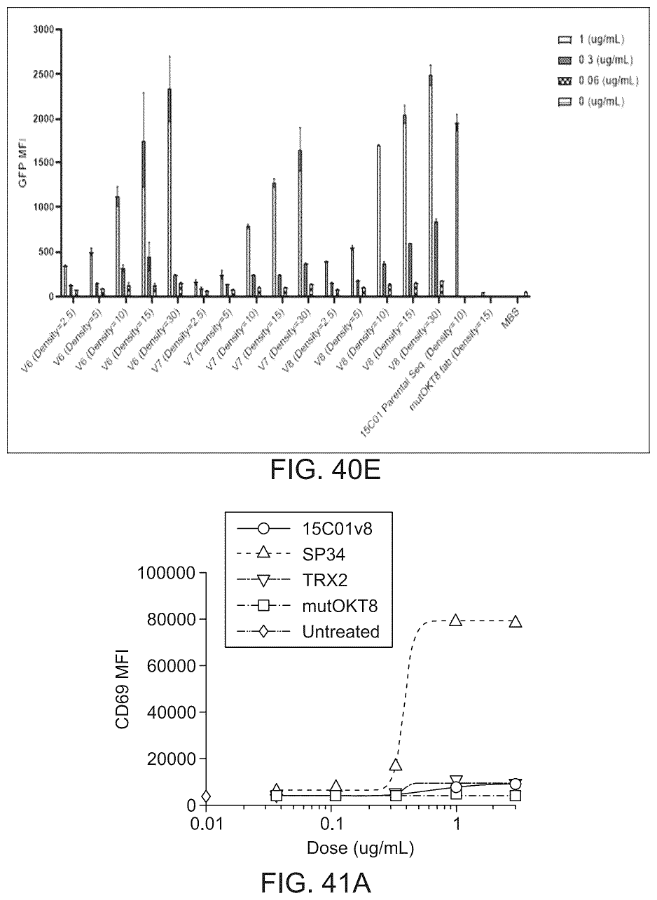

This example compares the viability, Dil association, and GFP protein expression resulting from Lipid 15 LNPs with different optimized variants of the 15C01 &CD8-targeting moiety (SEQ ID 1, SEQ ID 7, SEQ ID 8, SEQ ID 9). Nanoparticles bearing GFP encoding mRNA (and a fluorescent dye label (DiI-C18-5DS)) were produced using the microfluidic mixing and buffer exchange processes described in Example 45. Different variants of 15C01 (SEQ ID 1, SEQ ID 7, SEQ ID 8, SEQ ID 9) αCD8 VHH-conjugate was incorporated into the parent LNPs to obtain the final antibody targeted LNP formulation using the process described in Example 48. Particles thus produced were tested in vitro in primary human CD8+ T cells to assess reported gene expression as described in Example 51. All 15C01 targeting variants tested showed a similar level of T-cell viability to the untreated control 24 hours post-transfection (FIG. 40A). At all dose levels, the 15C01 variants tested showed comparable levels of LNP association in terms of the fraction of DiI+ T-cells (reflected by % DiI+ cells) (FIG. 40B). However, the amount of LNP association per cell (reflected by DiI MFI values) varied between the variants with the 15C01v8 (SEQ ID 9) variant showing the highest signal at a density of 10 VHHs per LNP at the 1 μg/mL dose (FIG. 40C and Table 39).

TABLE 39
DiI MFI values of Lipid 15 LNPs targeted with 15C01 variants (SEQ ID 1, SEQ ID 7,
SEQ ID 8, SEQ ID 9) at different densities and treated at various mRNA doses.

| | | | | | | | | | Fold/ | Fold/ | Fold/ |
|---|---|---|---|---|---|---|---|---|---|---|---|
| | | | | | | | | | background | background | background |
| | | | | | | | | | (1.0 | (0.3 | (0.06 |
| DiI MFI | 1 (μg/mL) | | 0.3 (μg/mL) | | 0.06 (μg/mL) | | 0 (μg/mL) | | μg/mL) | μg/mL) | μg/mL) |
| V6 (SEQ ID 7) | 27215 | 26637 | 3163 | 3077 | 494 | 317 | | | 723.8 | 83.9 | 10.9 |
| (Density = 2.5) | | | | | | | | | | | |
| V6 (SEQ ID 7) | 40150 | 29944 | 4556 | 3878 | 1038 | 796 | | | 942.1 | 113.4 | 24.7 |
| (Density = 5) | | | | | | | | | | | |
| V6 (SEQ ID 7) | 94371 | 73704 | 18808 | 13296 | 4425 | 2283 | | | 2259.1 | 431.5 | 90.2 |
| (Density = 10) | | | | | | | | | | | |
| V6 (SEQ ID 7) | 135072 | 109337 | 28410 | 20137 | 3547 | 3201 | | | 3285.1 | 652.5 | 90.7 |
| (Density = 15) | | | | | | | | | | | |
| V6 (SEQ ID 7) | 125773 | 135404 | 8392 | 7629 | 4453 | 3823 | | | 3510.4 | 215.3 | 111.2 |
| (Density = 30) | | | | | | | | | | | |
| V7 (SEQ ID 8) | 7237 | 7782 | 1627 | 1801 | 399 | 335 | | | 201.9 | 46.1 | 9.9 |
| (Density = 2.5) | | | | | | | | | | | |
| V7 (SEQ ID 8) | 14618 | 17988 | 3612 | 3663 | 713 | 524 | | | 438.3 | 97.8 | 16.6 |
| (Density = 5) | | | | | | | | | | | |
| V7 (SEQ ID 8) | 51272 | 59904 | 10945 | 11560 | 2860 | 1874 | | | 1494.3 | 302.5 | 63.6 |
| (Density = 10) | | | | | | | | | | | |
| V7 (SEQ ID 8) | 99358 | 119739 | 10318 | 11351 | 2310 | 2091 | | | 2944.9 | 291.3 | 59.2 |
| (Density = 15) | | | | | | | | | | | |
| V7 (SEQ ID 8) | 87469 | 94371 | 14926 | 15171 | 3547 | 3113 | | | 2444.1 | 404.5 | 89.5 |
| (Density = 30) | | | | | | | | | | | |
| V8 (SEQ ID 9) | 19622 | 19948 | 2957 | 3777 | 666 | 534 | | | 531.9 | 90.5 | 16.1 |
| (Density = 2.5) | | | | | | | | | | | |
| V8 (SEQ ID 9) | 31414 | 27739 | 5381 | 4575 | 1152 | 1481 | | | 795.1 | 133.8 | 35.4 |
| (Density = 5) | | | | | | | | | | | |
| V8 (SEQ ID 9) | 146860 | 143996 | 16769 | 21768 | 3208 | 2940 | | | 3909.4 | 518.0 | 82.6 |
| (Density = 10) | | | | | | | | | | | |
| V8 (SEQ ID 9) | 113718 | 139119 | 25337 | 32879 | 4176 | 3619 | | | 3398.3 | 782.5 | 104.8 |
| (Density = 15) | | | | | | | | | | | |
| V8 (SEQ ID 9) | 120329 | 114841 | 33037 | 35167 | 4099 | 4211 | | | 3160.9 | 916.7 | 111.7 |
| (Density = 30) | | | | | | | | | | | |
| 15C01 Parental | 112883 | 120922 | | | | | | | 3142.5 | | |
| Seq. (SEQ ID | | | | | | | | | | | |
| 1) | | | | | | | | | | | |
| (Density = 10) | | | | | | | | | | | |
| mutOKT8 fab | 75.8 | 71.9 | | | | | | | 2.0 | | |
| (Density = 15) | | | | | | | | | | | |
| MBS | | | | | | | 38.5 | 35.9 | | | |

At dose levels<1 μg/mL, the 15C01v8 variant at a density of 30 VHHs per LNP showed increased levels of GFP expression in terms of the fraction of GFP+ T-cells (reflected by % GFP+ cells) (FIG. 40D) and in terms of the copy number of GFP molecules produced per cell as reflected by the GFP MFI values (FIG. 40E and Table 40).

TABLE 40
GFP MFI values of Lipid 15 LNPs targeted with 15C01 variants
at different densities and treated at various mRNA doses.

| | | | | | | | | | Fold/ | Fold/ | Fold/ |
|---|---|---|---|---|---|---|---|---|---|---|---|
| | | | | | | | | | background | background | background |
| GFP MFI | 1 (ug/mL) | | 0.3 (ug/mL) | | 0.06 (ug/mL) | | 0 (ug/mL) | | (1.0 ug/mL) | (0.3 ug/mL) | (0.06 ug/mL) |
| V6 (SEQ ID 7) | 351 | 344 | 130 | 134 | 74.5 | 69.4 | | | 6.2 | 2.4 | 1.3 |
| (Density = 2.5) | | | | | | | | | | | |
| V6 (SEQ ID 7) | 531 | 471 | 152 | 146 | 92.5 | 88.7 | | | 9.0 | 2.7 | 1.6 |
| (Density = 5) | | | | | | | | | | | |
| V6 (SEQ ID 7) | 1199 | 1042 | 348 | 294 | 149 | 118 | | | 20.1 | 5.7 | 2.4 |
| (Density = 10) | | | | | | | | | | | |
| V6 (SEQ ID 7) | 2136 | 1381 | 562 | 336 | 144 | 109 | | | 31.5 | 8.0 | 2.3 |
| (Density = 15) | | | | | | | | | | | |
| V6 (SEQ ID 7) | 2080 | 2593 | 238 | 249 | 142 | 160 | | | 41.8 | 4.4 | 2.7 |
| (Density = 30) | | | | | | | | | | | |
| V7 (SEQ ID 8) | 142 | 183 | 89.9 | 104 | 59.1 | 69.4 | | | 2.9 | 1.7 | 1.2 |
| (Density = 2.5) | | | | | | | | | | | |
| V7 (SEQ ID 8) | 211 | 281 | 140 | 138 | 82.2 | 77.1 | | | 4.4 | 2.5 | 1.4 |
| (Density = 5) | | | | | | | | | | | |
| V7 (SEQ ID 8) | 770 | 801 | 246 | 237 | 111 | 100 | | | 14.1 | 4.3 | 1.9 |
| (Density = 10) | | | | | | | | | | | |
| V7 (SEQ ID 8) | 1235 | 1305 | 248 | 241 | 103 | 105 | | | 22.7 | 4.4 | 1.9 |
| (Density = 15) | | | | | | | | | | | |
| V7 (SEQ ID 8) | 1475 | 1828 | 376 | 363 | 143 | 135 | | | 29.6 | 6.6 | 2.5 |
| (Density = 30) | | | | | | | | | | | |
| V8 (SEQ ID 9) | 388 | 396 | 149 | 160 | 87.4 | 80.9 | | | 7.0 | 2.8 | 1.5 |
| (Density = 2.5) | | | | | | | | | | | |
| V8 (SEQ ID 9) | 564 | 524 | 186 | 175 | 99 | 107 | | | 9.7 | 3.2 | 1.8 |
| (Density = 5) | | | | | | | | | | | |
| V8 (SEQ ID 9) | 1705 | 1712 | 363 | 387 | 144 | 127 | | | 30.6 | 6.7 | 2.4 |
| (Density = 10) | | | | | | | | | | | |
| V8 (SEQ ID 9) | 1979 | 2123 | 596 | 597 | 162 | 148 | | | 36.7 | 10.7 | 2.8 |
| (Density = 15) | | | | | | | | | | | |
| V8 (SEQ ID 9) | 2568 | 2410 | 862 | 833 | 178 | 177 | | | 44.6 | 15.2 | 3.2 |
| (Density = 30) | | | | | | | | | | | |
| 15C01 Parental | 1895 | 2025 | | | | | | | 35.1 | | |
| Seq. (SEQ ID) | | | | | | | | | | | |
| 1) | | | | | | | | | | | |
| (Density = 10) | | | | | | | | | | | |
| mutOKT8 fab | 57.8 | 57.8 | | | | | | | 1.0 | | |
| (Density = 15) | | | | | | | | | | | |
| MBS | | | | | | | 56.5 | 55.2 | | | |

### Example 71. CD69 Expression in Human CD8+ T-Cells Transfected with Lipid 15 LNPs Containing AntiCD3 (SP34) and AntiCD8 (15C01V8 and TRX2) Targeting Moieties

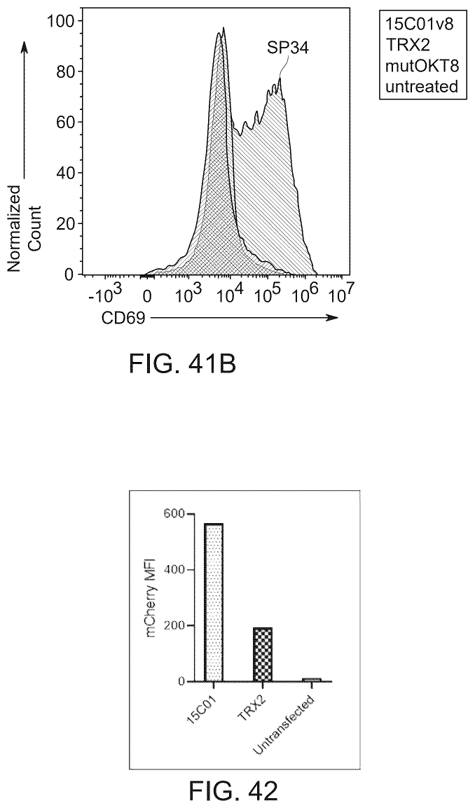

This example compares the expression levels of the early T-cell activation marker, CD69, resulting from Lipid 15 LNPs with αCD8-targeting moieties (TRX2 and 15C01v8) and αCD3-targeting moiety (SP34). Nanoparticles bearing GFP encoding mRNA (and a fluorescent dye label (DiI-C18-5DS)) were produced using the microfluidic mixing and buffer exchange processes described in Example 45. αCD8 and αCD3 Fab- or VHH-conjugate was incorporated into the parent LNPs to obtain the final antibody targeted LNP formulation using the process described in Example 48. Particles thus produced were tested in vitro in primary human CD8+ T cells to assess expression levels of CD69 as described in Example 51 and Example 52. Transfection with SP34-targeted LNPs showed elevated levels of CD69 expression compared to antiCD8 (15C₀₁v8 and TRX2) targeting moieties at all dose levels reflected by the CD69 MFI (FIG. 41A, FIG. 41B, and Table 41). This indicates that targeting the CD3 receptor results in T-cell activation, which is not a result of targeting CD8.

TABLE 41
GFP MFI values of Lipid 15 LNPs targeted with 15C01v8 variants
at different densities and treated at various mRNA doses.

| CD69 MFI | 3 ug/mL | 1 ug/mL | 0.33 ug/mL | 0.11 ug/mL | 0.037 ug/mL | 0 ug/mL |
|---|---|---|---|---|---|---|
| 15C01v8 (SEQ ID 9) | 9143 | 7795 | 4768 | 4216 | 3870 | |
| mutOKT8 | 4247 | 4416 | 4036 | 4273 | 4078 | |
| SP34 | 78825 | 79433 | 17543 | 8680 | 6574 | |
| TRX2 | 8803 | 10187 | 4732 | 4468 | 4158 | |
| Untreated | | | | | | 3923 |

### Example 72. mCherry Expression in NHP CD8+ T-Cells Transfected with Lipid 15 LNPs Containing Different AntiCD8 (15C01 or TRX2) Targeting Moieties

This example compares the mCherry protein expression in primary NHP CD8+ T-cells resulting from Lipid 15 LNPs with αCD8-targeting moieties (15C01 and TRX2). Nanoparticles bearing mCherry encoding mRNA were produced using the microfluidic mixing and buffer exchange processes described in Example 45. Different αCD8-conjugates (15C01 and TRX2) were incorporated into the parent LNPs to obtain the final antibody targeted LNP formulation using the process described in Example 48. Particles thus produced were tested in vitro in primary NHP CD8+ T cells to assess reported gene expression as described in Example 57. Transfection efficiency was increased with 15C01-targeted LNPs over TRX2-targeted LNPs and the untreated control in terms of the mCherry MFI values (FIG. 42). This indicates that the 15C01 targeting moiety is cross-reactive and can thus bind and be internalized by NHP CD8+ cells.

### Example 73. CD22 CAR (Expression in NHP CD8+ Cells Transfected with Lipid 15 and KC3 LNPs Containing AntiCD8 (15C01 (SEQ ID 1)) Targeting Moiety

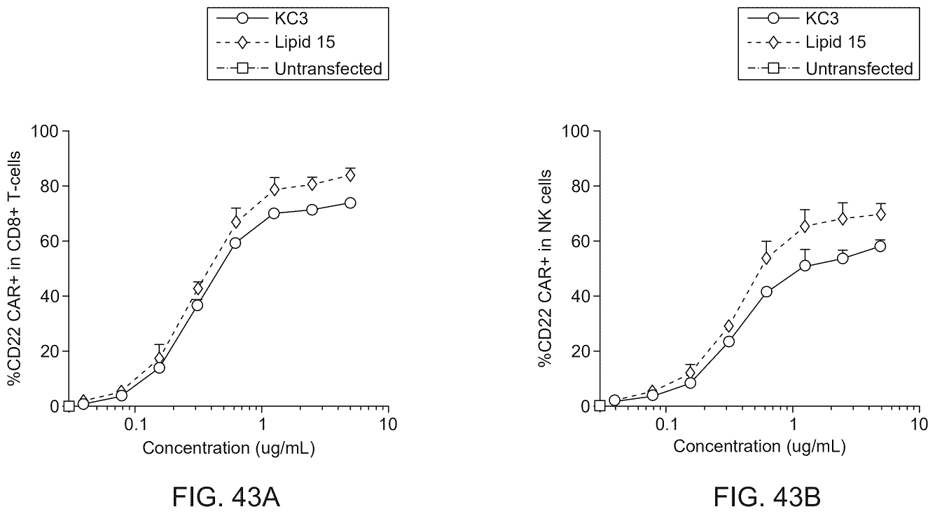

This example compares the CD22 CAR protein expression in primary NHP CD8+ cells resulting from Lipid 15 and KC3 LNPs with an αCD8-targeting moiety (15C01 (SEQ ID 1)). Nanoparticles bearing CD22 CAR encoding mRNA were produced using the microfluidic mixing and buffer exchange processes described in Example 45. The αCD8 VHH 15C01 conjugate was incorporated into the parent LNPs to obtain the final antibody targeted LNP formulation using the process described in Example 48. Particles thus produced were tested in vitro in primary NHP CD8+ cells to assess CD22 CAR expression as described in Example 57. The level of CD22 CAR expression was assessed using the method described in Example 54. Transfection efficiency was slightly increased with Lipid 15 LNPs over KC3 LNPs in both CD8+ T-cells and CD8+NK cells in terms of the % CAR+ cells (FIG. 43A, FIG. 43B, Table 42, and Table 43) at all mRNA dose levels.

TABLE 42
% CAR+ of CD8+ T-cells transfected with Lipid 15 or KC3
LNPs targeted with 15C01 (SEQ ID 1) at various mRNA doses.

| % CAR in NHP | | | | | | |
|---|---|---|---|---|---|---|
| CD8+ T-cells | KC3 | | Lipid 15 | | Untransfected | |
| 0.039 ug/mL | 1.54 | 0.54 | 2.58 | 1.66 | | |
| 0.078 ug/mL | 4.19 | 3.88 | 6.52 | 4.56 | | |
| 0.156 ug/mL | 15.1 | 13.3 | 20.6 | 14.2 | | |
| 0.313 ug/mL | 38.3 | 35.5 | 44.4 | 41.5 | | |
| 0.625 ug/mL | 59.4 | 60.1 | 70.6 | 63.9 | | |
| 1.25 ug/mL | 69.2 | 71.6 | 81.7 | 76.4 | | |
| 2.5 ug/mL | 72.4 | 71.5 | 82.5 | 79.4 | | |
| 5 ug/mL | 73.9 | 74.4 | 85.9 | 82.6 | | |
| 0 ug/mL | | | | | 0.13 | 0.12 |

TABLE 43
% CAR+ of CD8+ NK cells transfected with Lipid 15 or KC3
LNPs targeted with 15C01 (SEQ ID 1) at various mRNA doses.

| % CAR in NHP | | | | | | |
|---|---|---|---|---|---|---|
| NK cells | KC3 | | lipid 15 | | Untransfected | |
| 0.039 ug/mL | 1.78 | 2.23 | 3.44 | 1.41 | | |
| 0.078 ug/mL | 4.49 | 3.62 | 5.39 | 5.47 | | |
| 0.156 ug/mL | 8.44 | 8.55 | 14.1 | 10.1 | | |
| 0.313 ug/mL | 23.5 | 24 | 30 | 28.4 | | |
| 0.625 ug/mL | 42.7 | 40.7 | 58.1 | 49.8 | | |
| 1.25 ug/mL | 55.5 | 47 | 69.7 | 61.3 | | |
| 2.5 ug/mL | 56.1 | 51.7 | 72.2 | 64.4 | | |
| 5 ug/mL | 56.8 | 60.2 | 72.4 | 67.4 | | |
| 0 ug/mL | | | | | 0.55 | 0 |

### Example 74. In Vitro Viability, and CD22 Car Expression (TTR-102 (SEQ ID 139)) in Primary Human T-Cells Transfected with aCD8 (15C01 (SEQ ID 1)) Targeted LNPs Based on Lipid 15 and Comparator Lipid DLn-KC3-DMA after 1-5 Freeze-Thaw Cycles

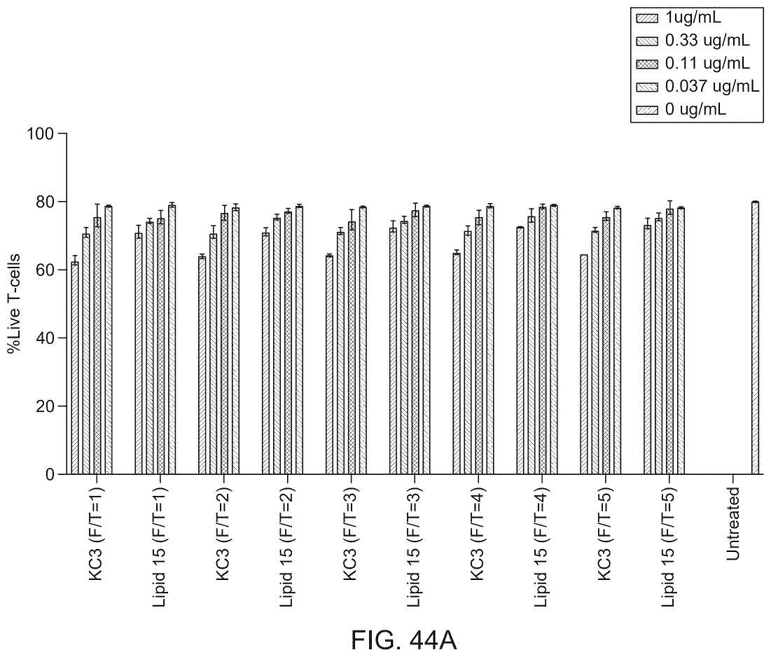

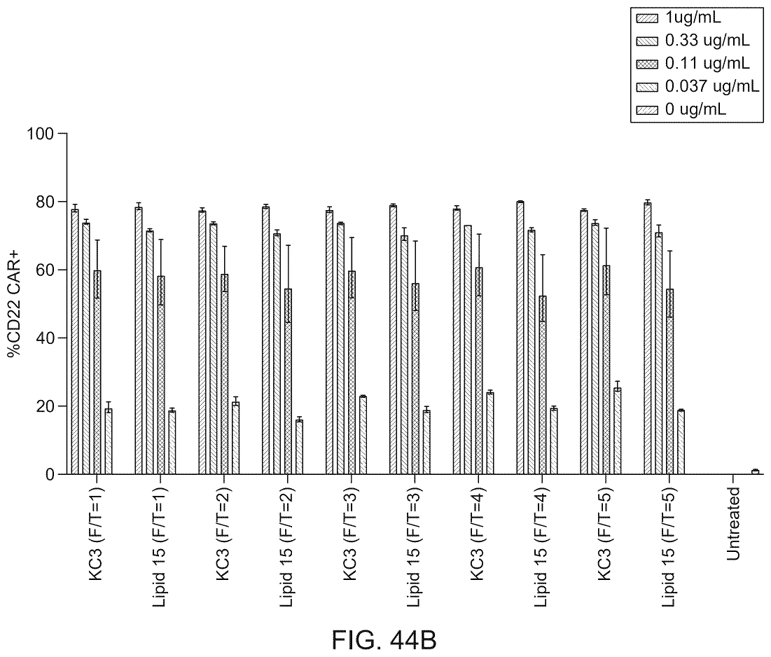

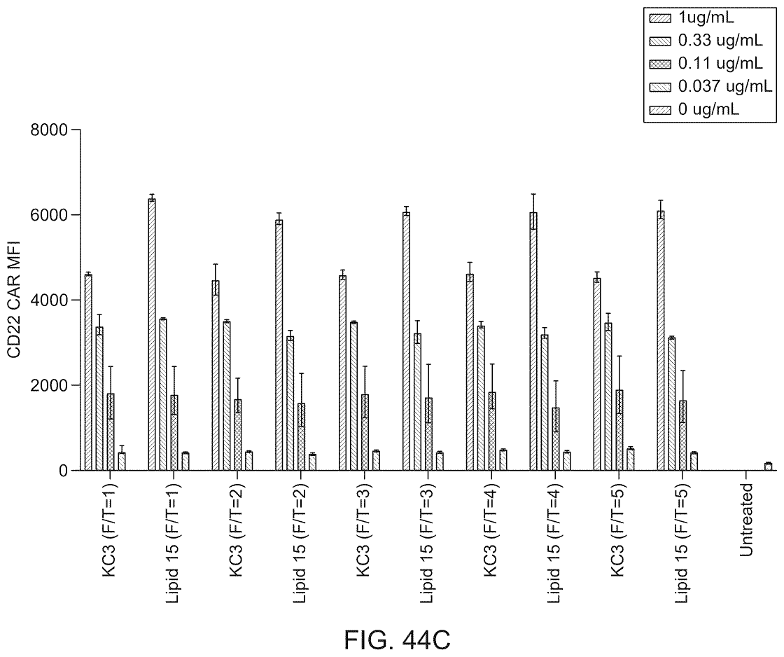

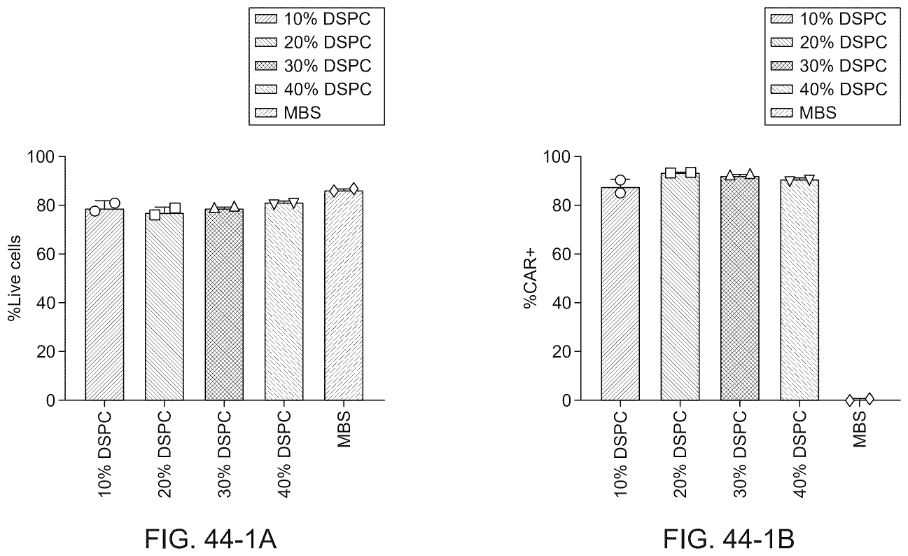

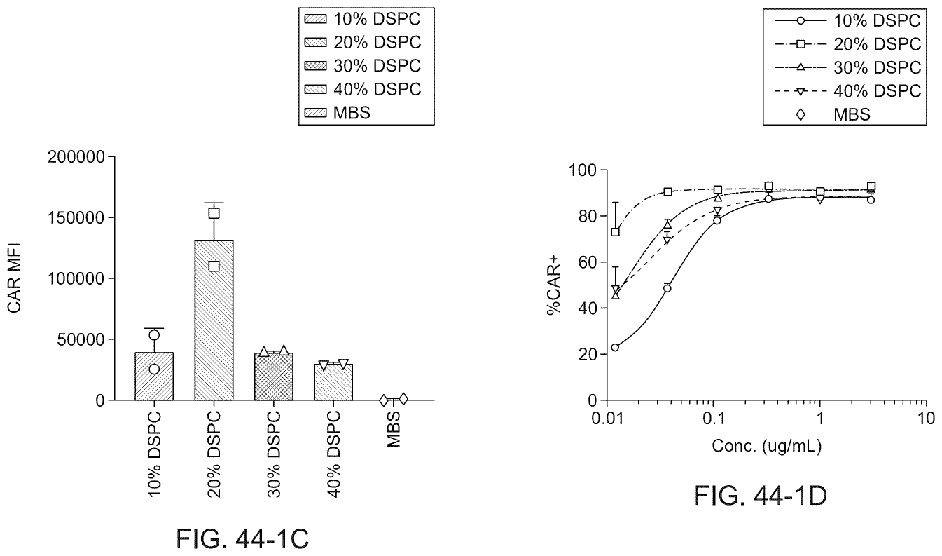

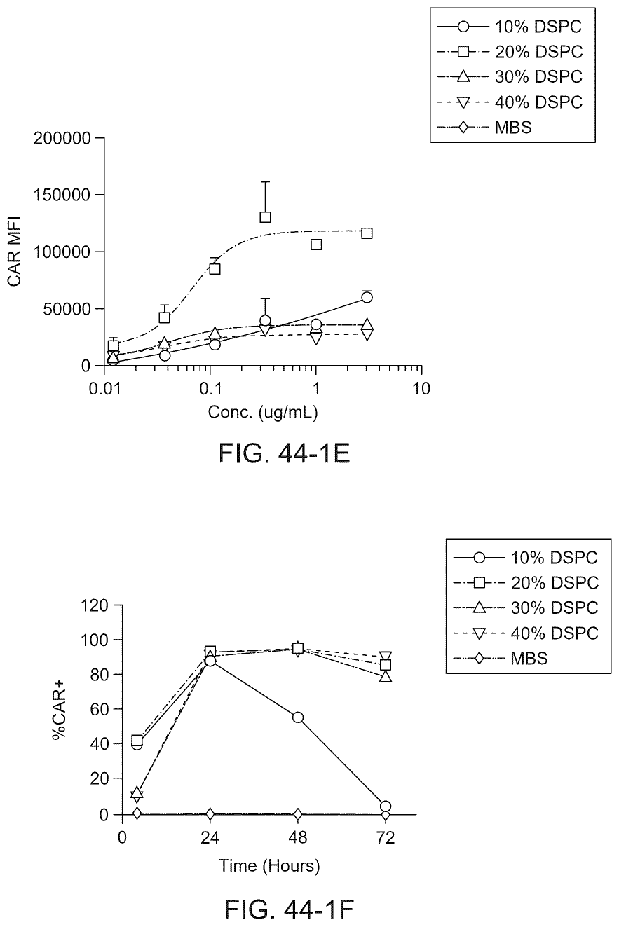

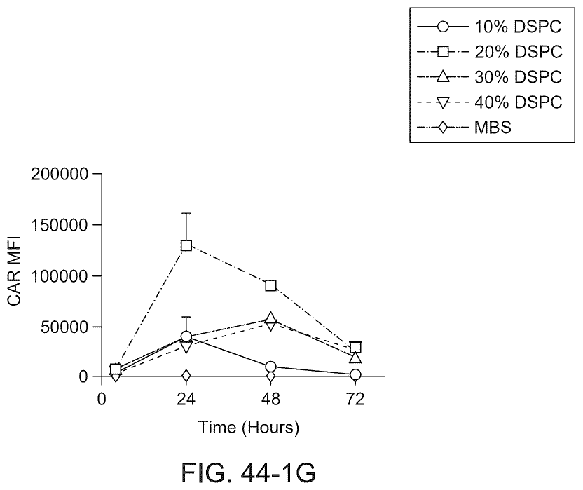

This example compares the CD22 CAR (TTR-102 (SEQ ID 139)) protein expression in primary human CD8+ cells resulting from Lipid 15 and KC3 LNPs with an &CD8-targeting moiety (15C01 (SEQ ID 1)) after up to 5 freeze-thaw cycles. Nanoparticles bearing CD22 CAR encoding mRNA were produced using the microfluidic mixing and buffer exchange processes described in Example 45. The αCD8 VHH conjugate (comprising 15C01 (SEQ ID 1)) was incorporated into the parent LNPs to obtain the final VHH-targeted LNP formulation using the process described in Example 48. Particles thus produced were tested in vitro in primary human CD8+ cells to assess CAR expression as described in Example 51. The level of CD22 CAR expression was assessed using the method described in Example 54. As shown in FIG. 44A, no change in T-cell viability could be observed between the five freeze-thaw cycles. At all dose levels, both Lipid 15 and KC3 LNPs did not show a decrease in transfection efficiency between the five freeze-thaw cycles both in terms of the fraction of CD22 CAR+ T-cells (reflected by % CD22 CAR+ cells) (FIG. 44B) and the copies of CAR protein produced on a per cell basis (reflected by CD22 CAR MFI values) (FIG. 44C). Thus, the performance of Lipid 15 and KC3 LNPs was well preserved after up to five freeze-thaw cycles.

### Example 74-1. In Vitro Viability, and CD22 Car Expression ((SEQ ID 147)) in Primary Human T-Cells Transfected with aCD8 (15C01V8) Targeted LNPs Based on Lipid 15 with Varying DSPC Content

This example compares the CD22 CAR ((SEQ ID 147)) protein expression in primary human CD8+ cells resulting from Lipid 15 LNPs with varying % DSPC content and with an αCD8-targeting moiety (15C01v8). Nanoparticles bearing CD22 CAR encoding mRNA were produced using the microfluidic mixing and buffer exchange processes described in Example 45 but with varying amounts of DSPC (Table 43-1).

TABLE 43-1
LNP formulations with varying levels of DSPC.

| | 10% | 20% | 30% | 40% |
|---|---|---|---|---|
| | DSPC | DSPC | DSPC | DSPC |
| Lipid 15 (mol %) | 49.24 | 49.24 | 49.24 | 49.24 |
| Cholesterol (mol %) | 39.40 | 29.22 | 19.21 | 9.29 |
| DSPC (mol %) | 9.85 | 20.03 | 30.04 | 39.95 |
| DPG-PEG(2000) (mol %) | 1.51 | 1.51 | 1.51 | 1.51 |

The αCD8 VHH conjugate (15C01v8) was incorporated into the parent LNPs to obtain the final VHH-targeted LNP formulation using the process described in Example 48. Particles thus produced were tested in vitro in primary human CD8+ cells to assess CAR expression as described in Example 51. The level of CD22 CAR expression was assessed using the method described in Example 54. As shown in FIG. 44-1A, no change in T-cell viability could be observed between the LNP groups with varying DSPC content. After 24 hours with a 0.33 μg/mL mRNA dose no difference in CAR expression was observed between the LNP groups with varying DSPC content in terms of the fraction of CD22 CAR+ T-cells (reflected by % CD22 CAR+ cells) (FIG. 44-1B). However, a difference was observed in the copies of CAR protein produced on a per cell basis (reflected by CD22 CAR MFI values) where the 20% DSPC group was superior to the other formulations tested (FIG. 44-1C). Similarly, the 20% DSPC group outperformed the other LNPs tested in a dose titration both in terms of the faction of CD22 CAR+ T-cells (reflected by % CD22 CAR+ cells) (FIG. 44-1D) and in terms of CAR protein produced on a per cell basis (reflected by CD22 CAR MFI values) (FIG. 44-1E). Finally, the 20% DSPC group outperformed the other LNPs tested in a time course both in terms of the faction of CD22 CAR+ T-cells (reflected by % CD22 CAR+ cells) (FIG. 44-1F) and in terms of CAR protein produced on a per cell basis (reflected by CD22 CAR MFI values) (FIG. 44-1G).

### Example 75. Illustration of CD22 Car Cassette Designs

This example shows the designs of the chimeric antigen receptors (CARs) (FIG. 45). Each anti-CD22 binder, in a ScFv format with a 3× (G4S) linker between the VH and VL domain, was incorporated into four different CAR cassettes containing varying extracellular hinge domains. The first CAR cassette was designed to incorporate an extracellular hinge domain derived from human CD28 being 40 amino acids in length (CAR Cassette #1). The three other CAR cassettes contained extracellular hinges derived from the human CD8alpha (CD8α) domain with varying lengths being 26 (CAR Cassette #2), 46 (CAR Cassette #3), and 66 (CAR Cassette #4) amino acids, respectively. Intracellularly, the CAR cassettes contained a CD28 co-stimulatory domain and a signaling domain derived from CD3zeta.

### Example 76. CD22 Car Expression in HEK293T Cells Transfected with Car Plasmid DNA Encoding Various AntiCD22 Clones and Car Cassettes

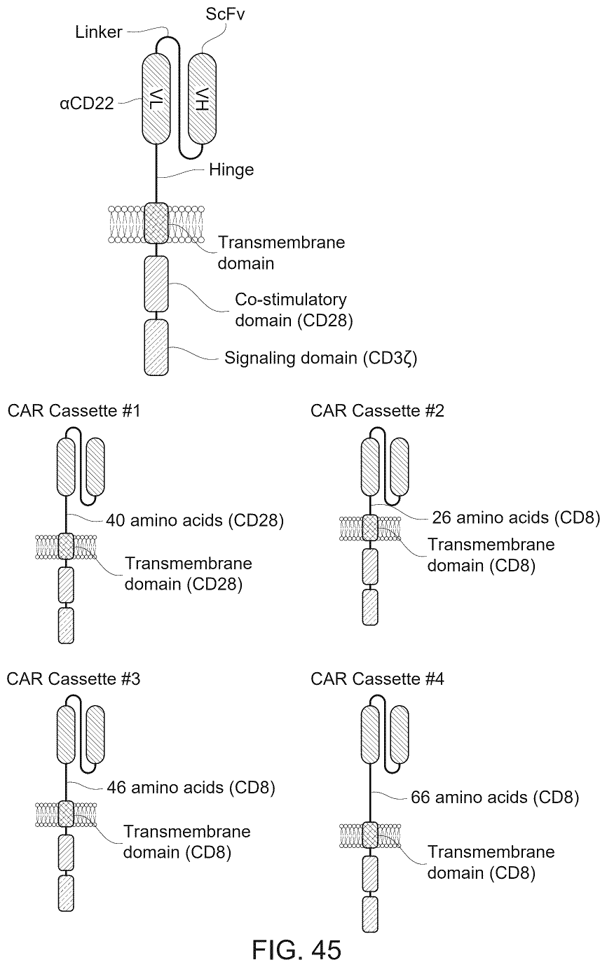

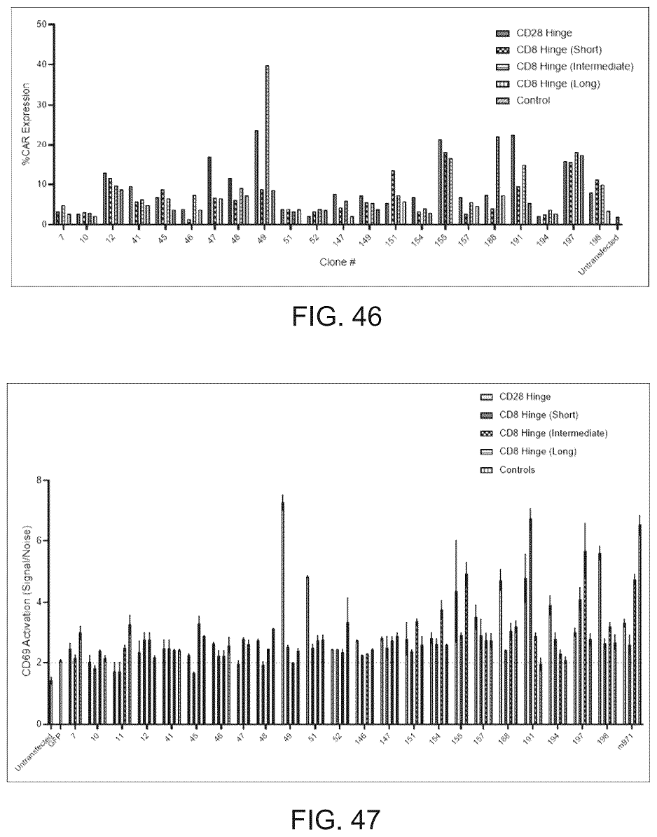

This example compares the CD22 CAR protein expression levels in HEK293T cells resulting from CAR plasmid DNA transfection. DNA plasmids encoding various CD22 CARs were transfected into HEK293 Ts as described in Example 58. The level of CD22 CAR expression was assessed using the method described in Example 54. Increased CAR expression was observed in HEK293 Ts transfected with a plasmid encoding clone 49 incorporated into a CAR cassette containing a CD28-derived hinge domain of 40 amino acids in length and with a plasmid encoding clone 49 incorporated into a CAR cassette containing a CD8alpha-derived hinge domain of 46 amino acids in length (FIG. 46).

### Example 77. CD22 CAR-Mediated CD69 Activation in Jurkat Cells Transfected with Car Plasmid DNA Encoding Various AntiCD22 Clones and Car Cassettes

This example compares the CD69 signal in Jurkat cells resulting from CAR plasmid DNA transfection and co-culture with CD22-expressing Raji cells. DNA plasmids encoding various CD22 CARs were transfected into Jurkat cells and co-cultured with Raji cells as described in Example 59. The level of CD69 expression was assessed as described in Example 52. Increased CD69 expression was observed in Jurkat cells transfected with a plasmid encoding clone 49 incorporated into a CAR cassette containing a CD28-derived hinge domain of 40 amino acids in length and with a plasmid encoding clone 191 incorporated into a CAR cassette containing a CD8alpha-derived hinge domain of 26 amino acids in length (FIG. 47). The NCI m971 clone was used as a benchmark control and showed increased CD69 upregulation when incorporated into a CD8alpha-derived hinge domain of 66 amino acids in length (FIG. 47).

### Example 78. CD22 Car Expression in Jurkat Cells Transfected with mRNA Encoding Various AntiCD22 Car Constructs

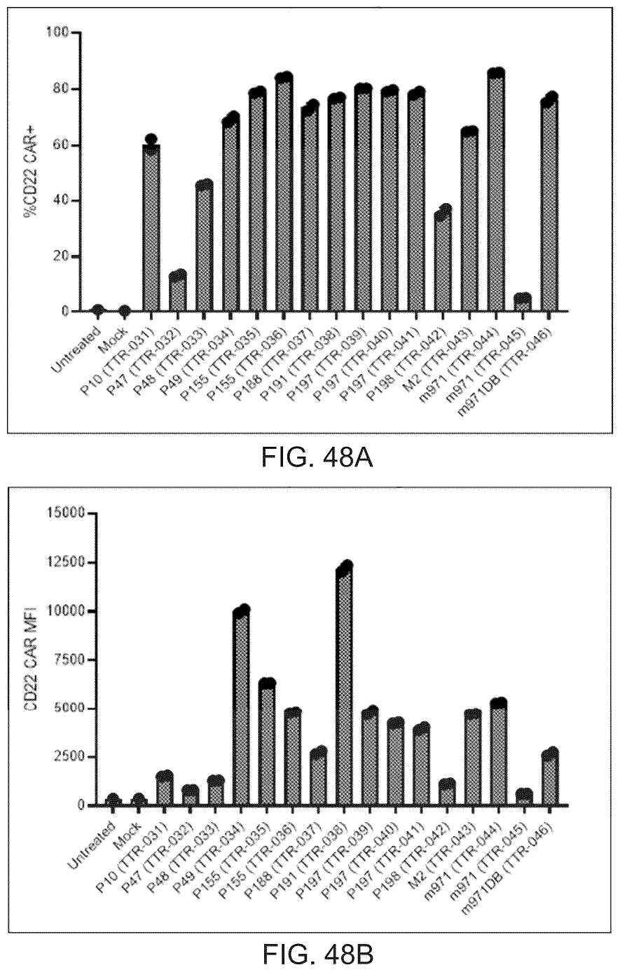

This example compares the CD22 CAR protein expression levels in Jurkat cells resulting from CAR mRNA transfection. mRNA encoding various CD22 CAR constructs were transfected into Jurkat cells as described in Example 60. The level of CD22 CAR expression was assessed using the method described in Example 54. A lower fraction of Jurkat cells expressed CAR protein when transfected with mRNA encoding clones 47, 48, and 198 as illustrated by the % CD22 CAR+ cells (FIG. 48A). However, Jurkat cells transfected with mRNA encoding clones 49 and 191 showed increased CAR expression (>20-fold increase over background) in terms of the copy number of CAR molecules produced per cell as reflected by the CAR MFI values (FIG. 48B and Table 44).

TABLE 44
CAR MFI of Jurkat cells transfected with mRNA encoding
various CD22 CAR constructs by electroporation.

| | | Fold/background |
|---|---|---|
| CAR MFI | 1 μg/million cells | (1 ug/million cells) |

| | | | |
|---|---|---|---|
| Untreated | 362 | | |
| Mock | 373 | | |
| P10 (TTR-031) | 1491 | 1554 | 4.1 |
| P47 (TTR-032) | 813 | 799 | 2.2 |
| P48 (TTR-033) | 1291 | 1301 | 3.5 |
| P49 (TTR-034) | 10112 | 9918 | 26.8 |
| (SEQ ID 128) | | | |
| P155 (TTR-035) | 6334 | 6346 | 17.0 |
| (SEQ ID 129) | | | |
| P155 (TTR-036) | 4791 | 4841 | 12.9 |
| (SEQ ID 130) | | | |
| P188 (TTR-037) | 2799 | 2647 | 7.3 |
| P191 (TTR-038) | 12044 | 12362 | 32.7 |
| P197 (TTR-039) | 4933 | 4709 | 12.9 |
| (SEQ ID 131) | | | |
| P197 (TTR-040) | 4275 | 4221 | 11.4 |
| (SEQ ID 132) | | | |
| P197 (TTR-041) | 4028 | 3880 | 10.6 |
| (SEQ ID 133) | | | |
| P198 (TTR-042) | 1154 | 1108 | 3.0 |
| (SEQ ID 134) | | | |
| M2 (TTR-043) | 4711 | 4752 | 12.7 |
| (SEQ ID 135) | | | |
| m971 (TTR-044) | 5302 | 5345 | 14.3 |
| (SEQ ID 136) | | | |
| m971 (TTR-045) | 615 | 621 | 1.7 |
| (SEQ ID 137) | | | |
| m971DB (TTR-046) | 2741 | 2566 | 7.1 |
| (SEQ ID 138) | | | |

### Example 79. CD22 CAR Expression in Primary Human T-Cells Transfected with mRNA Encoding Various AntiCD22 CAR Constructs

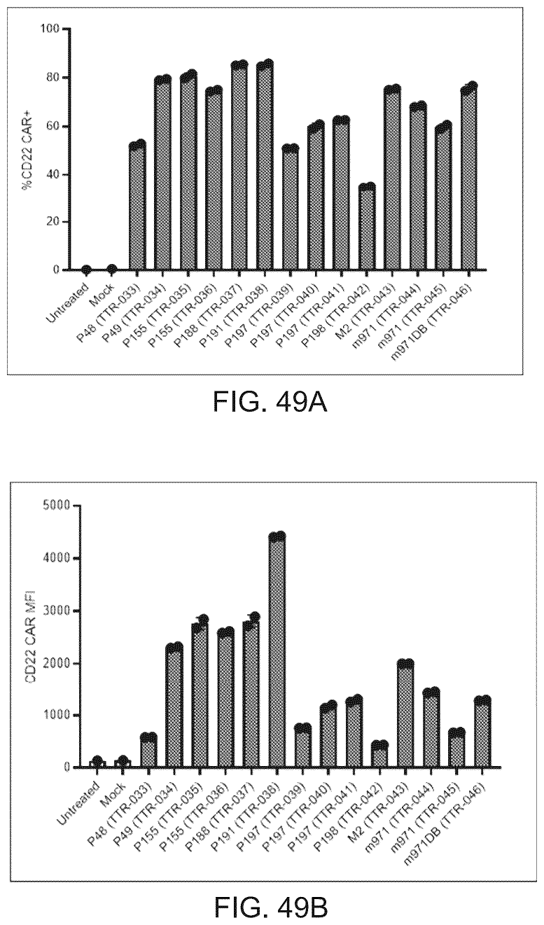

This example compares the CD22 CAR protein expression levels in primary human T-cells resulting from CAR mRNA transfection. mRNA encoding various CD22 CAR constructs were transfected into human T-cells as described in Example 61. The level of CD22 CAR expression was assessed using the method described in Example 54. mRNA encoding the NCI m971 clone was used as a benchmark control. An increased fraction of T-cells expressed CAR protein when transfected with mRNA encoding clones 49, 155, 188, 191, and M2 as illustrated by the % CD22 CAR+ cells (FIG. 49A). Similarly, T-cells transfected with mRNA encoding clones 49, 155, 188, 191, and M2 showed increased CAR expression (>15-fold increase over background) in terms of the copy number of CAR molecules produced per cell as reflected by the CAR MFI values (FIG. 49B and Table 45).

TABLE 45
CAR MFI of primary human T-cells transfected with mRNA encoding
various CD22 CAR constructs by electroporation.

| | | | Fold/background |
|---|---|---|---|
| CAR MFI | CD22 CARs | | (1 ug/million cells) |
| Untreated | 138 | | |
| Mock | 143 | | |
| P48 (TTR-033) | 581 | 586 | 4.1 |
| P49 (TTR-034) | 2329 | 2300 | 16.2 |
| (SEQ ID 128) | | | |
| P155 (TTR-035) | 2847 | 2681 | 19.3 |
| (SEQ ID 129) | | | |
| P155 (TTR-035) | 2586 | 2617 | 18.2 |
| (SEQ ID 139) | | | |
| P188 (TTR-037) | 2896 | 2725 | 19.7 |
| P191 (TTR-038) | 4427 | 4404 | 30.9 |
| P197 (TTR-039) | 757 | 764 | 5.3 |
| (SEQ ID 131) | | | |
| P197 (TTR-040) | 1135 | 1199 | 8.2 |
| (SEQ ID 132) | | | |
| P197 (TTR-041) | 1306 | 1255 | 9.0 |
| (SEQ ID 133) | | | |
| P198 (TTR-042) | 432 | 438 | 3.0 |
| (SEQ ID 134) | | | |
| M2 (TTR-043) | 2004 | 2002 | 14.0 |
| (SEQ ID 135) | | | |
| m971 (TTR-044) | 1427 | 1451 | 10.1 |
| (SEQ ID 136) | | | |
| m971 (TTR-045) | 667 | 677 | 4.7 |
| (SEQ ID 137) | | | |
| m971DB (TTR-046) | 1293 | 1279 | 9.0 |
| (SEQ ID 138) | | | |

### Example 80. Cytotoxicity of Raji Cells Co-Cultured with Human T-Cells Electroporated with mRNA Encoding Various CD22 CAR Constructs

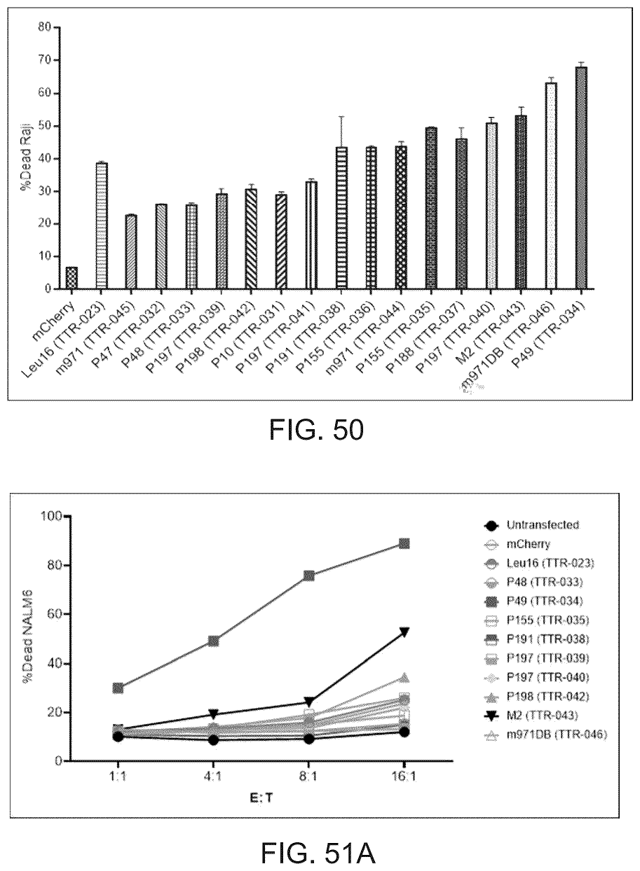

This example compares the CAR-mediated cytotoxicity against Raji cells resulting from CAR mRNA transfection by electroporation and subsequent co-culture with CD22-expressing Raji cells. mRNA encoding various CD22 CAR constructs were transfected into primary human T-cells, co-cultured with Raji cells, and the level of cytotoxicity was assessed as described in Example 61 and Example 52. The NCI m971 clone was used as a benchmark control. The highest level of CAR-mediated cytotoxicity (>65%) was observed in T-cells transfected with mRNA encoding clone 49 incorporated into a CAR cassette containing a CD28-derived hinge domain of 40 amino acids in length (FIG. 50 and Table 46).

TABLE 46
CAR-mediated cytotoxicity of Raji cells co-cultured
with human T-cells transfected with mRNA encoding
various CD22 CAR constructs by electroporation.

| % Dead Raji | E:T = 16:1 | |
|---|---|---|
| mCherry | 6.76 | 6.85 |
| Leu16 (TTR-023) (SEQ ID 159) | 39.3 | 38.4 |
| m971 (TTR-045) (SEQ ID 137) | 22.3 | 23 |
| P47 (TTR-032) | 26.4 | 25.6 |
| P48 (TTR-033) | 26.6 | 24.9 |
| P197 (TTR-039) (SEQ ID 131) | 28.1 | 31 |
| P198 (TTR-042) (SEQ ID 134) | 29.4 | 32.3 |
| P10 (TTR-031) | 30 | 28.5 |
| P197 (TTR-041) (SEQ ID 133) | 32.6 | 34 |
| P191 (TTR-038) | 34.5 | 52.9 |
| P155 (TTR-036) (SEQ ID 130) | 44 | 43.6 |
| m971 (TTR-044) (SEQ ID 136) | 45.3 | 42.7 |
| P155 (TTR-035) (SEQ ID 129) | 49.5 | 49.8 |
| P188 (TTR-037) | 49.5 | 42.8 |
| P197 (TTR-040) (SEQ ID 132) | 49.5 | 52.7 |
| M2 (TTR-043) (SEQ ID 135) | 55.8 | 51.1 |
| m971DB (TTR-046) (SEQ ID 138) | 62 | 64.8 |
| P49 (TTR-034) (SEQ ID 128) | 66.7 | 69.5 |

### Example 81. Cytotoxicity of Nalm6 and K562 Cells Co-Cultured with Human T-Cells Electroporated with CD22 Car mRNA Encoding Various CD22 Car Constructs

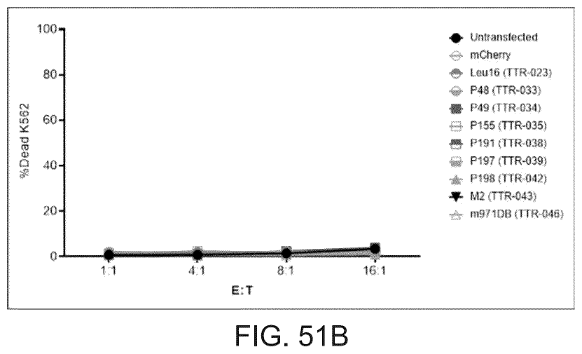

This example compares the CAR-mediated cytotoxicity against target-expressing Nalm6 cells and non-target-expressing K562 cells resulting from T-cells electroporated with CAR mRNA and subsequent co-culture with target cells. mRNA encoding various CD22 CAR constructs were transfected into primary human T-cells, co-cultured with Nalm6 or K562 cells, and the level of cytotoxicity was assessed as described in Example 61 and Example 52. The NCI m971 clone was used as a benchmark control. The highest level of CAR-mediated cytotoxicity against CD22-expressing Nalm6 cells was observed in T-cells transfected with mRNA encoding clone 49 incorporated into a CAR cassette containing a CD28-derived hinge domain of 40 amino acids in length at all effector-to-target ratios (E:Ts) tested (FIG. 51A). No cytotoxicity was observed against the non-target expressing K562 cell line for any of the CAR constructs tested (FIG. 51B).

### Example 82. Epitope Binning of Anti-CD22 Binders Against CD22

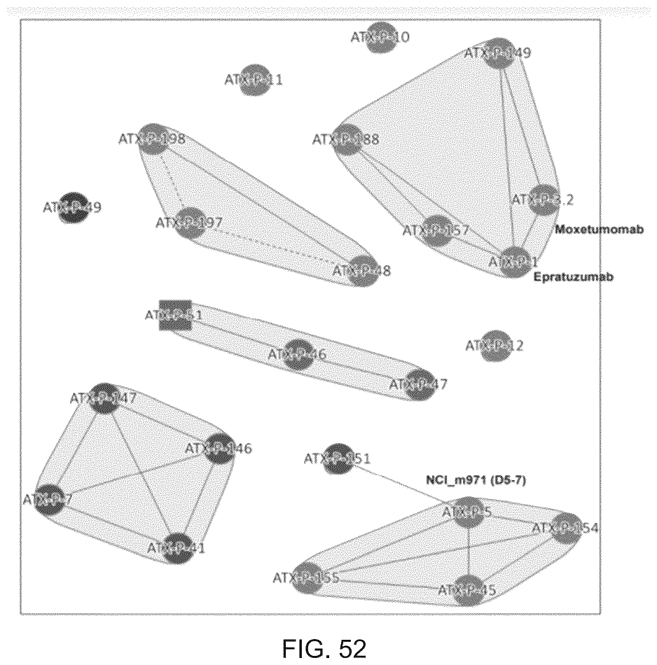

This example compares the epitope binning of anti-CD22 binders against CD22. The NCI m971 antibody, Epratuzumab, and Moxetumomab were used as binning controls. P49 was shown to be binding a unique epitope independent of other binders tested and P155 was binned with m971 (FIG. 52).

### Example 83. Binding of Human CD22 Cars to Non-Human Primate CD22 Antigen

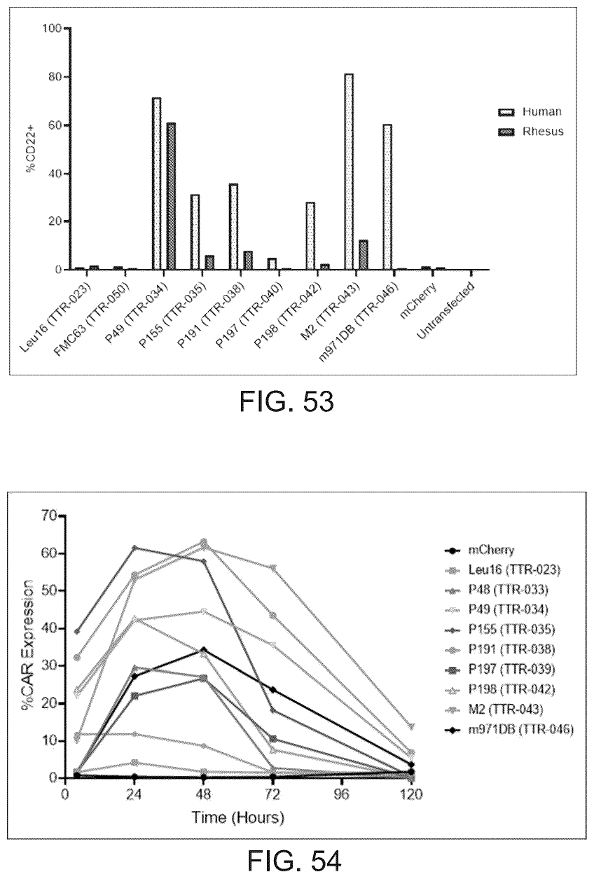

This example compares the binding of the CD22 CAR constructs to non-human primate CD22 antigen resulting from CAR mRNA transfection of primary human T-cells. mRNAs encoding various CD22 CAR constructs were transfected into primary human T-cells as described in Example 61. The level of NHP CD22 binding was assessed using the method described in Example 62 and compared to the level of human CD22 binding performed as described in Example 54. T-cells transfected with mRNA encoding clone 49 incorporated into a CAR cassette containing a CD28-derived hinge domain of 40 amino acids in length showed comparable (<2-fold difference) binding to rhesus CD22 antigen and human CD22 antigen indicating cross-reactivity of clone 49 with NHP CD22 antigen (FIG. 53 and Table 47).

TABLE 47
Fraction of T-cells, transfected with mRNA encoding
various CD22 CAR constructs, binding human CD22
antigen and Rhesus CD22 antigen, receptively.

| % CD22 CAR+ | Human | Rhesus | Human/Rhesus |
|---|---|---|---|
| Leu16 (TTR-023) (SEQ ID 329) | 1.19 | 1.77 | |
| P49 (TTR-034) (SEQ ID 128) | 71.7 | 61.2 | 1.2 |
| P155 (TTR-035) (SEQ ID 129) | 31.4 | 6.09 | 5.2 |
| P191 (TTR-038) | 36 | 7.9 | 4.6 |
| P197 (TTR-040) (SEQ ID 132) | 4.99 | 0.76 | 6.6 |
| P198 (TTR-042) (SEQ ID 134) | 28.1 | 2.52 | 11.2 |
| M2 (TTR-043) (SEQ ID 135) | 81.6 | 12.4 | 6.6 |
| m971DB (TTR-046) (SEQ ID 138) | 60.7 | 0.93 | 65.3 |
| mCherry | 1.42 | 1.03 | |
| Untransfected | 0.35 | 0.41 | |

### Example 84. CD22 CAR Expression in Primary Human T-Cells Transfected with mRNA Encoding AntiCD22 Car Constructs Over 120 Hours

This example compares the CD22 CAR protein expression levels over a time course of 120 hours in primary human T-cells resulting from CAR mRNA transfection. mRNA encoding various CD22 CAR constructs were transfected into human T-cells as described in Example 61. The level of CD22 CAR expression was assessed using the method described in Example 54. mRNA encoding the NCI m971 clone (SEQ ID 138) was used as a benchmark control. T-cells transfected with mRNA encoding clone 49 incorporated into a CAR cassette containing a CD28-derived hinge domain of 40 amino acids in length showed superior expression over other constructs at time points 72 and 120 hours indicating improved protein persistence on the cell surface compared to other CAR constructs tested (FIG. 54).

### Example 85. Cytokine Secretion of T-Cells Transfected with Lipid 15 LNPs Containing AntiCD8 (15C01 (SEQ ID 1)) Targeting Moiety and Encapsulating Various CAR mRNA Constructs

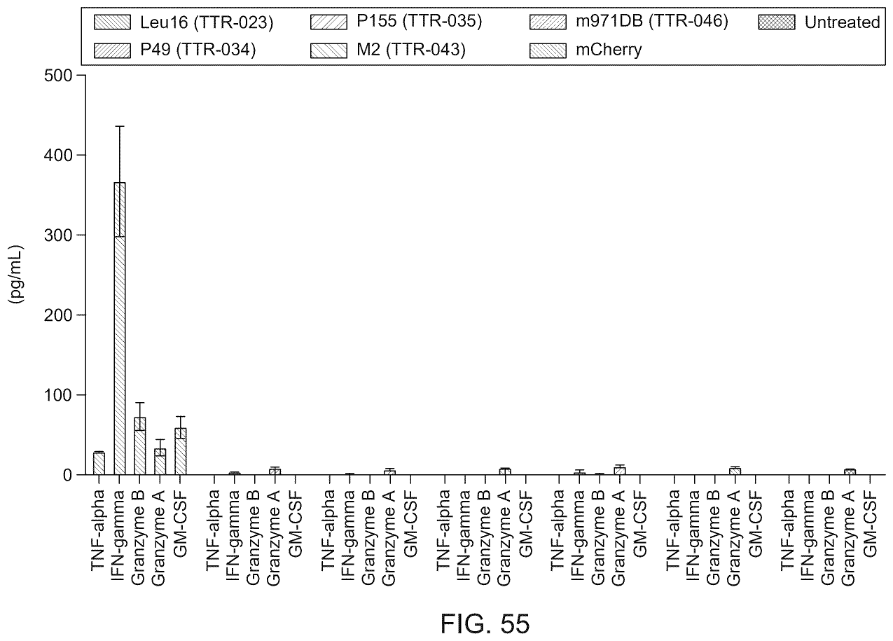

This example compares the level of cytokine secretion (TNF-alpha, IFN-gamma, Granzyme A, Granzyme B, and GM-CSF) in primary human T-cells resulting from CAR LNP transfection. Lipid 15 LNPs bearing CAR-encoding mRNA were produced using the microfluidic mixing and buffer exchange processes described in Example 45. An antiCD8 VHH 15C01 (SEQ ID 1)-conjugate (DSPE-PEG-VHH) was incorporated into the parent LNPs to obtain the final VHH-targeted LNP formulation using the process described in Example 48. Particles thus produced were tested in vitro in primary human CD8+ T cells as described in Example 51. The level of cytokines secreted into the supernatant was assessed by MSD using the method described in Example 53. T-cells transfected with the anti-CD20 CAR (TTR-023 (SEQ ID 159)) showed elevated levels of cytokines in the supernatant of all cytokines tested in comparison to other CAR constructs tested (FIG. 55). This suggests that the anti-CD20 CAR (TTR-023 (SEQ ID 159)) mediates tonic signaling once expressed on the T-cell surface, which is not observed for the other CAR constructs tested.

### Example 86. Expression and Quantification of CD22 on Various Cancer Cell Lines

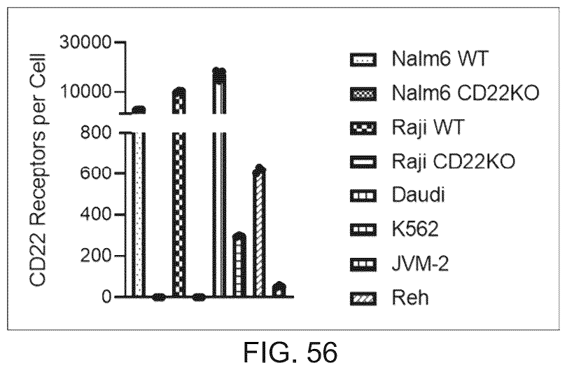

This example compares the levels of CD22 expression on various cancer cell lines. The antibodies bound per cell (ABC) of CD22 were quantified on eight different cell lines using the PE Quantibrite kit (BD) as described in Example 63. The Burkitt's Lymphoma cell line, Daudi, showed the highest expression of CD22 (^{~}17K/cell), and the CD22 knockout (KO) cell lines for Raji and Nalm6, respectively, showed no expression of CD22 (FIG. 56 and Table 48).

TABLE 48
Quantification of CD22 expression in different cancer cell lines.

| CD22 ABC | CD22 molecules per cell |
|---|---|
| Nalm6 WT | 2709 |
| Nalm6 CD22KO | 0 |
| Raji WT | 10026 |
| Raji CD22KO | 0 |
| Daudi | 17083 |
| K562 | 53 |
| JVM-2 | 294 |
| Reh | 617 |

### Example 87. In Vitro CAR-T Cell Function by Cancer Cell Line Co-Culture with aCD22 CAR (TTR-102 (SEQ ID 147)) Expressing T-Cells Derived by Transfection of Primary Human T-Cells with aCD8 (15C01v8 Seq Id 9) Targeted Lipid 15 LNPs Bearing CAR-mRNA or mCherry-mRNA (as Negative Control)

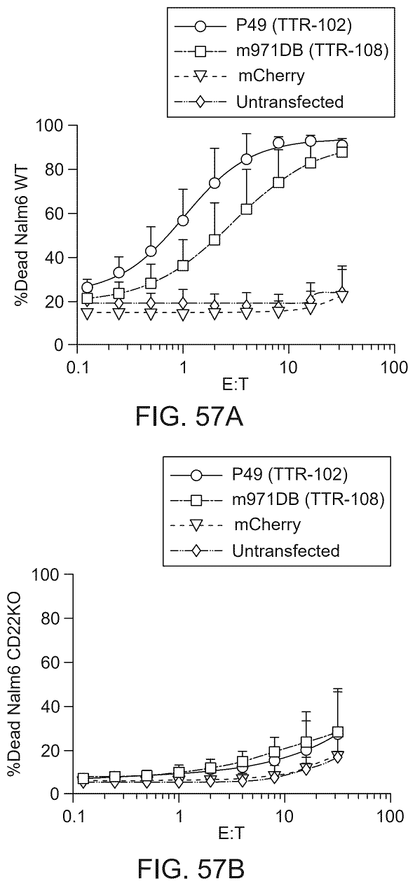

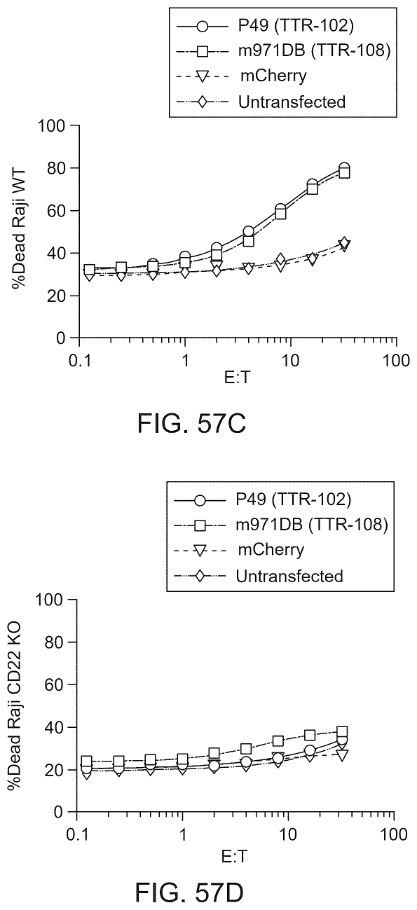

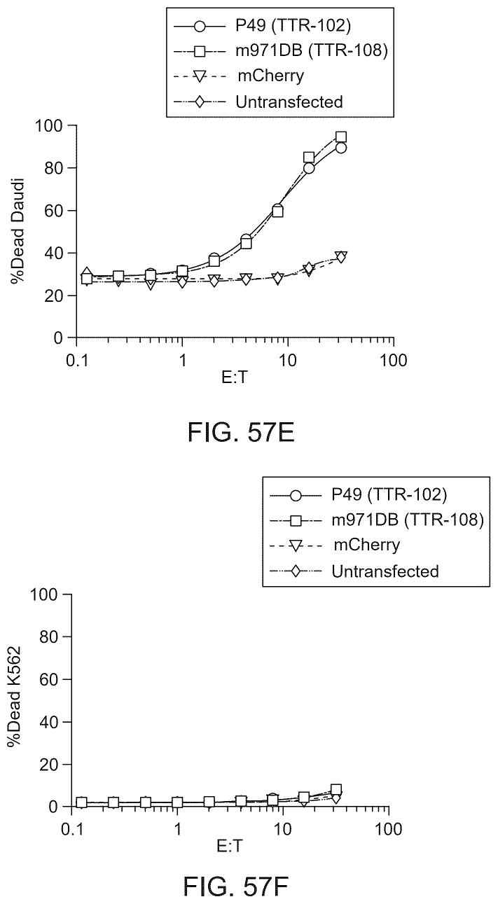

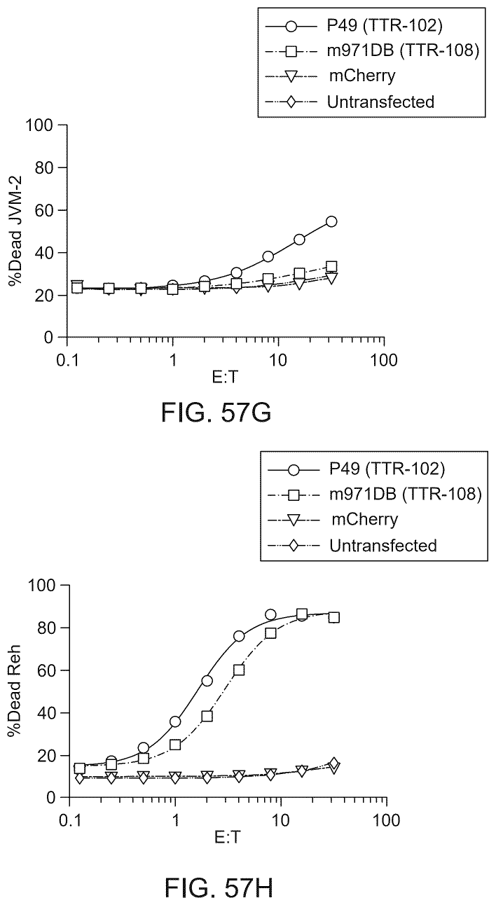

This example compares cytotoxicity resulting from Lipid 15 LNPs with inserted antiCD8 (15C01v8 (SEQ ID 9))-targeting moiety encapsulating various CAR-encoding mRNA payloads. Nanoparticles bearing CAR-encoding mRNA were produced using the microfluidic mixing and buffer exchange processes described in Example 45. An αCD8 VHH-conjugate was incorporated into the parent LNPs to obtain the final VHH-targeted LNP formulation using the process described in Example 48. Particles thus produced were tested in vitro in primary human CD8+ T cells co-cultured with target cancer cell lines to assess the CAR-mediated cytotoxicity as described in Example 56. The NCI antiCD22 m971 clone (SEQ ID 144) was used as a benchmark control. Increased levels of CAR-mediated cytotoxicity against wild-type Nalm6 cells (FIG. 57A), JVM-2 cells (FIG. 57G), and Reh cells (FIG. 57H) were observed for TTR-102 (SEQ ID 147) over the m971 (SEQ ID 144) benchmark control. This suggests that the TTR-102 (SEQ ID 147) CAR construct can promote stronger CAR-mediated cytotoxicity against CD22-expressing cell lines compared to the NCI m971 clone (SEQ ID 144). As expected, no cytotoxicity above background, as determined by untreated T-cells, was observed in the Nalm6 CD22 KO cell line (FIG. 57B), the Raji CD22 KO cell lines (FIG. 57D), and in the non-target expressing CML K562 cell line (FIG. 57F). Comparable CAR-mediated cytotoxicity was observed against the Raji cell line (FIG. 57C) and the Daudi cell line (FIG. 57E).

### Example 88. In Vitro CAR Expression in Human CD8+ T-Cells Transfected with Lipid 15 LNPs Containing an aCD8 (15C01V8 (SEQ ID 9)) Targeting Moiety and Encapsulating Various Car-Encoding mRNAs or mCherry Encoding mRNA

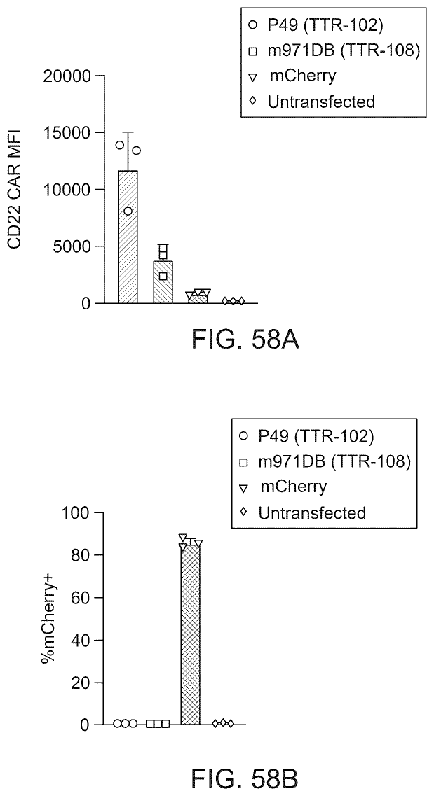

This example compares the CAR protein expression and reporter protein expression (mCherry) in primary human CD8+ T-cells resulting from Lipid 15 LNPs with an &CD8-targeting moiety (15C01v8 (SEQ ID 9)). Nanoparticles bearing CAR-encoding or mCherry-encoding mRNA were produced using the microfluidic mixing and buffer exchange processes described in Example 45. The αCD8 VHH conjugate (15C01v8 (SEQ ID 9)) was incorporated into the parent LNPs to obtain the final VHH-targeted LNP formulation using the process described in Example 48. Particles thus produced were tested in vitro in primary human CD8+ cells to assess CAR expression and reporter protein expression as described in Example 51. The level of CD22 CAR expression was assessed using the methods described in Example 54. TTR-102 (SEQ ID 147) showed increased expression over the NCI m971 (SEQ ID 144) control, which was used as a benchmark control, in all three donors in terms of CD22 CAR MFI expression (FIG. 58A). mCherry was observed in ~80% of the CD8+ T-cells for all three donors tested (FIG. 58B).

### Example 89. Repeated Car-Mediated Cytotoxicity of Nalm6 Cells Co-Cultured with Human CD8+ T-Cells Transfected with Lipid 15 LNPs Containing an aCD8 (15C01 (SEQ ID 1)) Targeting Moiety and Encapsulating CD22 Car-Encoding mRNA (TTR-102 (SEQ ID 139)) In Vitro

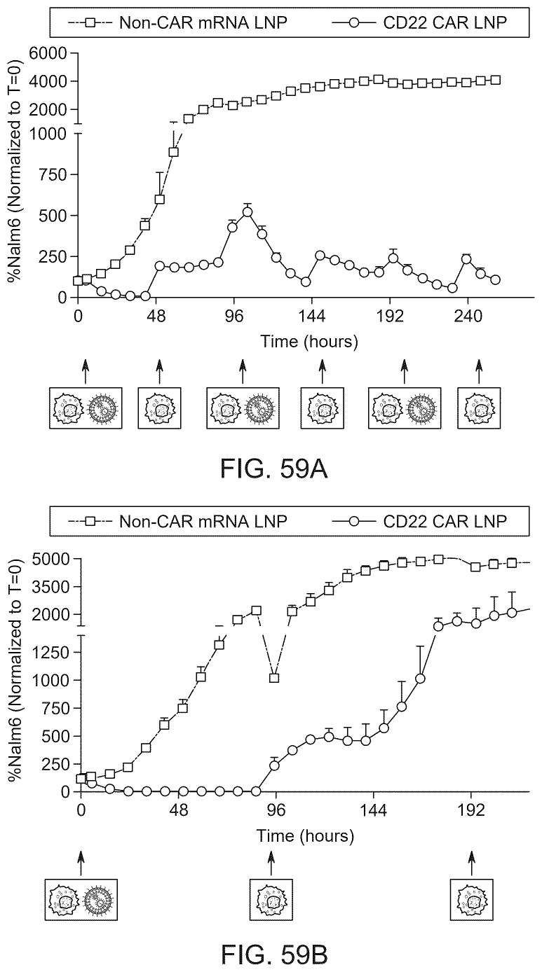

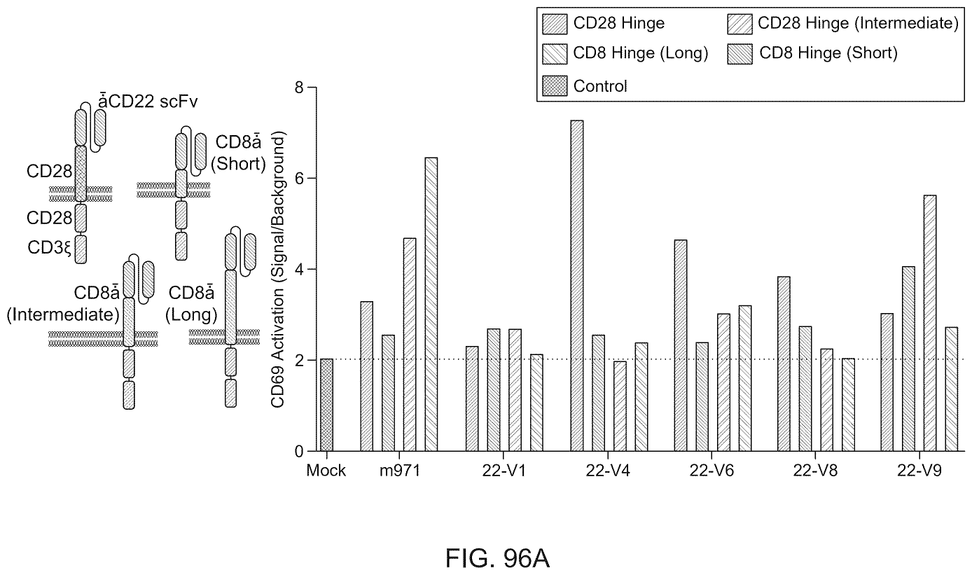

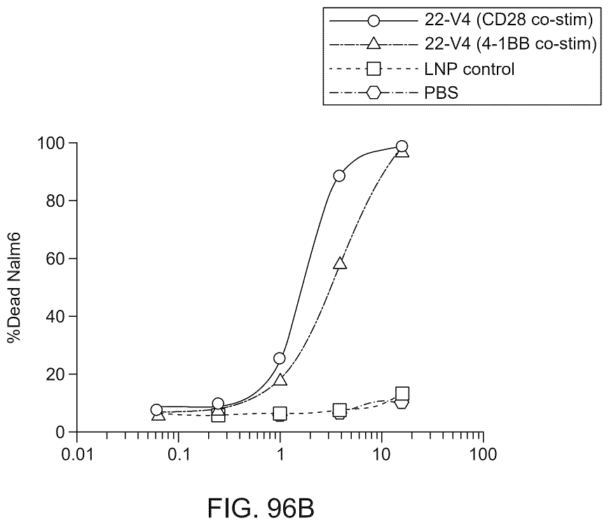

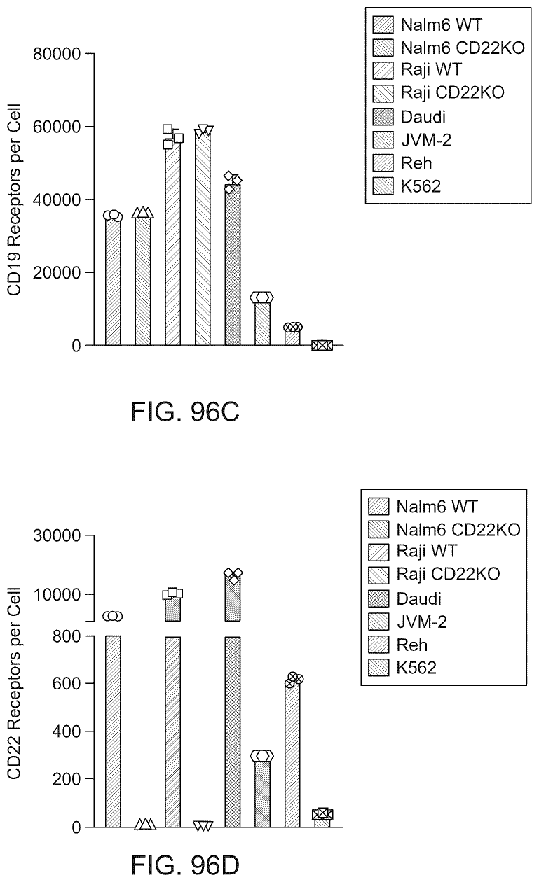

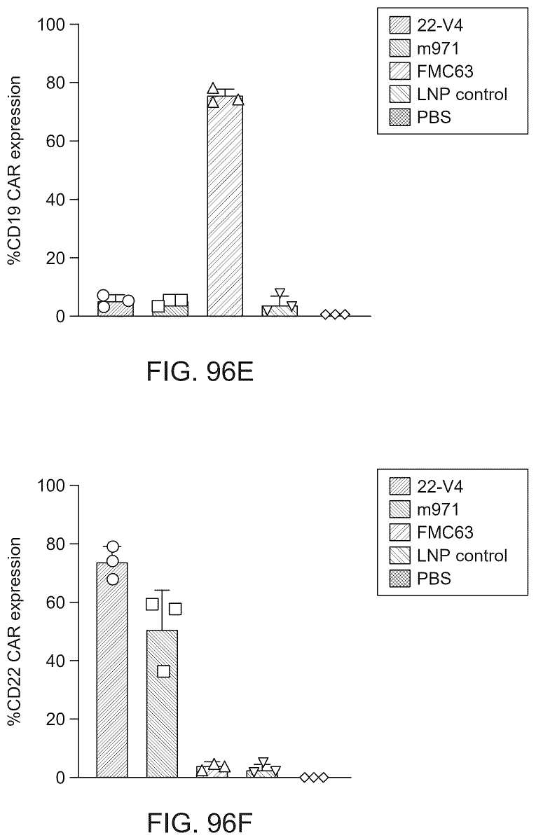

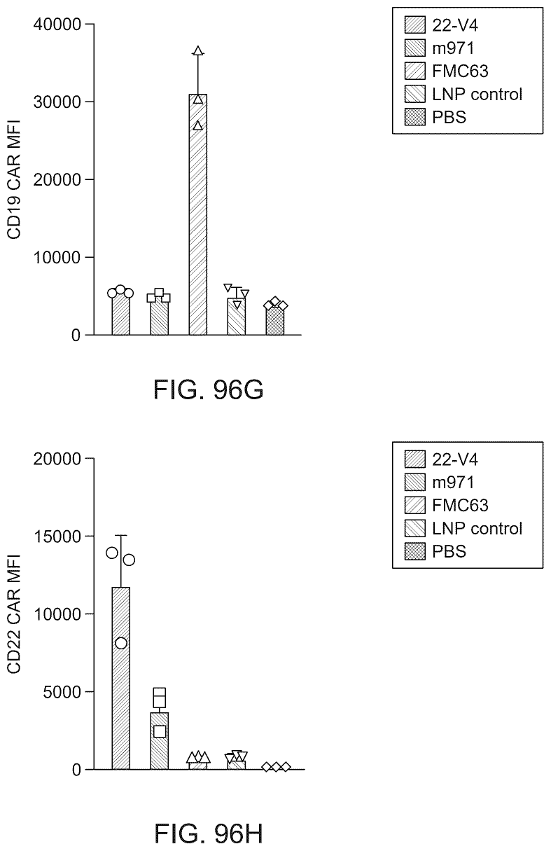

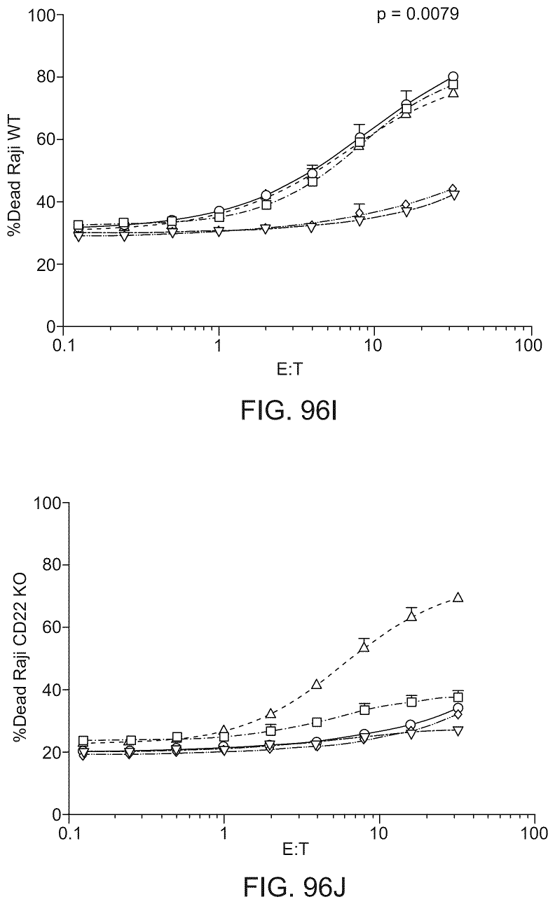

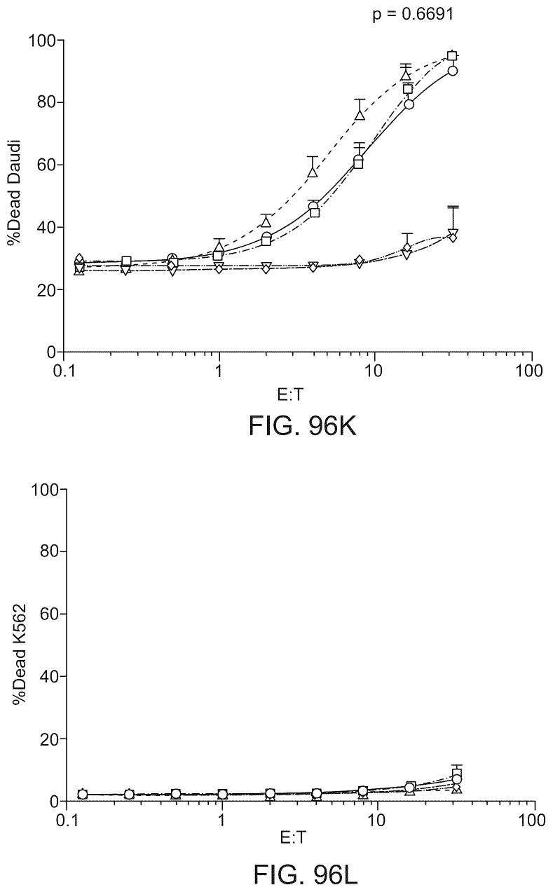

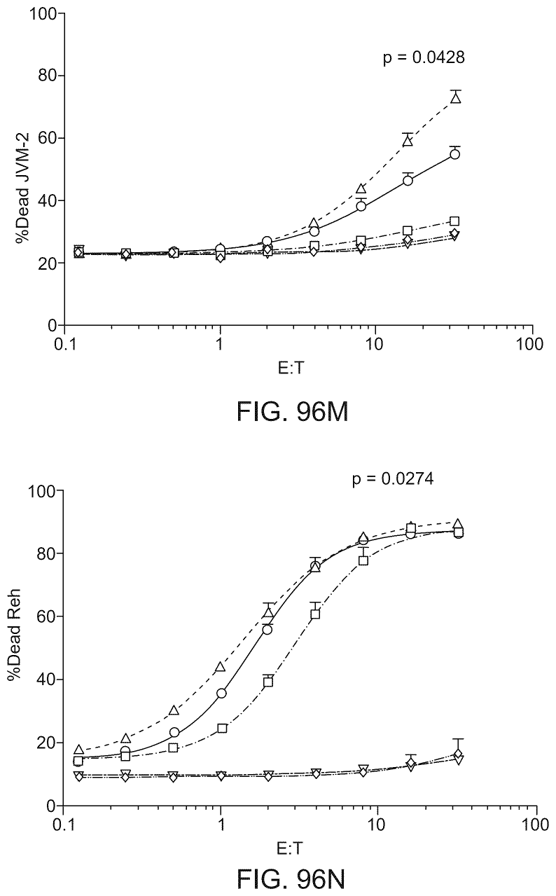

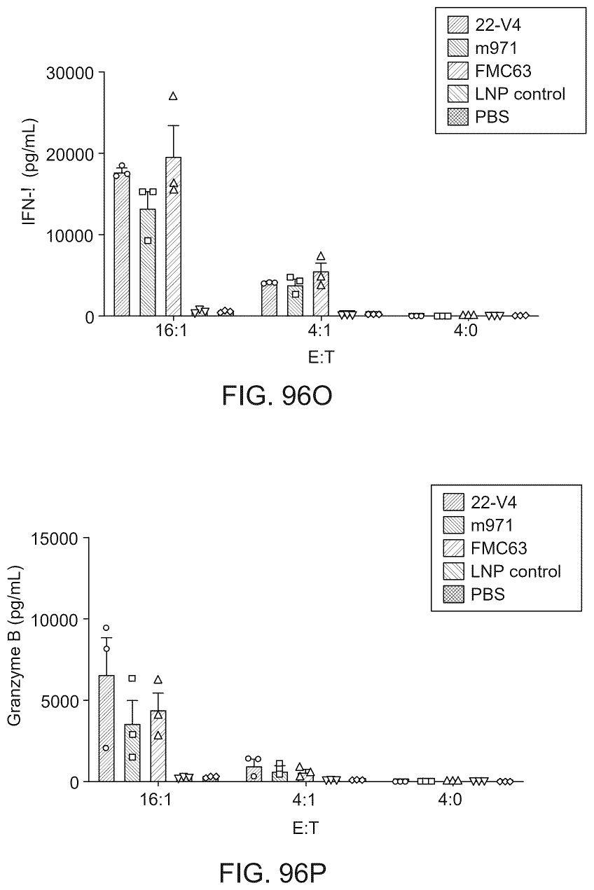

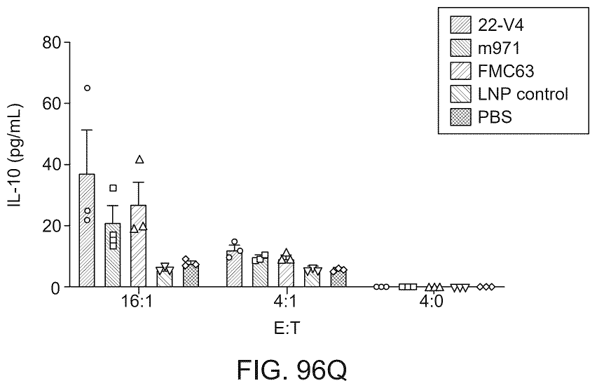

This example compares repeated CAR-mediated cytotoxicity of Nalm6 target cells resulting from co-culture with CD8+ T-cells after transfection with Lipid 15 LNPs inserted with a CD8-targeting moiety (15C01 (SEQ ID 1)). Nanoparticles bearing CAR-encoding mRNA (TTR-102 (SEQ ID 139)) were produced using the microfluidic mixing and buffer exchange processes described in Example 45. An αCD8 VHH-conjugate was incorporated into the parent LNPs to obtain the final VHH-targeted LNP formulation using the process described in Example 48. Particles thus produced were tested in vitro in primary human CD8+ T cells co-cultured with Nalm6 cells to assess CAR-mediated cytotoxicity as described in Example 56 but with the level of cytotoxicity being assessed at the various time points by utilizing the live cell imaging SX5 Incucyte system. Repeated addition of Nalm6 target cells to the culture was done every 48 hours (FIG. 59A) or 96 hours (FIG. 59B). Additionally, CAR LNPs were repeatedly added to the culture every 96 hours when target cells were added every 48 hours (FIG. 59A). With the re-transfection of CAR LNPs, the CAR-mediated cytotoxicity of Nalm6 cells was maintained after each addition for up to 240 hours (FIG. 59A). Without continuous transfection, CAR-mediated cytotoxicity could be observed for up to 192 hours (FIG. 59B).

### Example 90. In Vitro Car Expression in Human CD8+ T-Cells Transfected with Lipid 15 LNPs Containing an aCD8 (15C01 (SEQ ID 1)) Targeting Moiety and Encapsulating mRNA Encoding UTR- and Codon-Optimized Variants of CD22 Cars (TTR-102 (SEQ ID 139) and TTR-102 (SEQ ID 147) and TTR-121 (SEQ ID 146) and TTR-121 (SEQ ID 148))

This example compares the CAR protein expression in primary human CD8+ T-cells resulting from Lipid 15 LNPs with an αCD8-targeting moiety (15C01 (SEQ ID 1)). Nanoparticles bearing mRNA encoding different codon-optimized variants of TTR-102 (SEQ ID NO: 139 or SEQ ID NO: 147) and TTR-121 (SEQ ID NO: 146 or SEQ ID NO: 148) were produced using the microfluidic mixing and buffer exchange processes described in Example 45. The αCD8 VHH conjugate (15C01 (SEQ ID 1)) was incorporated into the parent LNPs to obtain the final VHH-targeted LNP formulation using the process described in Example 48. Particles thus produced were tested in vitro in primary human CD8+ cells to assess CAR expression as described in Example 51. The level of CD22 CAR expression was assessed using the method described in Example 54. Increased CD22 CAR expression could be observed for TTR-102 and TTR-121 containing Tidal UTRs (SEQ ID 139 and SEQ ID 146) over CoE UTRs (SEQ ID 147 and SEQ ID 148) both in terms of the fraction of CAR-expressing T-cells (FIG. 60A and Table 49) and in terms of CAR expression levels determined by CAR MFI (FIG. 60B and Table 50).

TABLE 49
Fraction of T-cells expressing CD22 CAR after transfection
with antiCD8 (15C01 (SEQ ID 1)) targeted LNPs based on Lipid
15 encapsulating mRNA encoding various UTR- and codon-optimized
variants of TTR-102 and TTR-121, respectively.

| % CAR+ | 0.3 ug/mL |
|---|---|
| TTR-102 (Tidal UTRs) | 88.7 |
| (TTR-102 (SEQ ID 139) | |
| TTR-102 (CoE UTRs) | 75.7 |
| (TTR-102 (SEQ ID 147), | |
| a.k.a., MRT14577) | |
| TTR-121 (Tidal UTRs) | 82.2 |
| (TTR-121 SEQ ID 146) | |
| TTR-121 (CoE UTRs) | 74.7 |
| (TTR-121 (SEQ ID 148), | |
| a.k.a., MRT14580) | |
| Fluc | 0.5 |
| PBS | 0.5 |

TABLE 50
CAR MFI expression of T-cells after transfection with antiCD8
(15C01 (SEQ ID 1)) targeted LNPs based on Lipid 15 encapsulating
mRNA encoding various UTR- and codon-optimized variants
of TTR-102 and TTR-121, respectively.

| | | | Fold/background |
|---|---|---|---|
| CAR MFI | 0.3 ug/mL | | (0.3 ug/mL) |
| TTR-102 (Tidal UTRs) | 1082 | 970 | 61.4 |
| (SEQ ID 139) | | | |
| TTR-102 (CoE UTRs) | 341 | 322 | 19.9 |
| (SEQ ID 147) | | | |
| TTR-121 (Tidal UTRs) | 642 | 491 | 33.9 |
| (SEQ ID 146) | | | |
| TTR-121 (CoE UTRs) | 309 | 350 | 19.7 |
| (SEQ ID 148) | | | |
| Fluc | 19.3 | 19.3 | |
| PBS | 16.7 | 16.7 | |

### Example 91. Cytotoxicity of Raji and Nalm6 Cells Co-Cultured with Human CD8+ T-Cells Transfected with Lipid 15 LNPs Containing an aCD8 (15C01 (SEQ ID 1)) Targeting Moiety and Encapsulating mRNA Encoding UTR- and Codon-Optimized Variants of CD22 CARS (TTR-102 (SEQ ID 139 and Seq Id 147) and TTR-121 (SEQ ID 146 and SEQ ID 148))

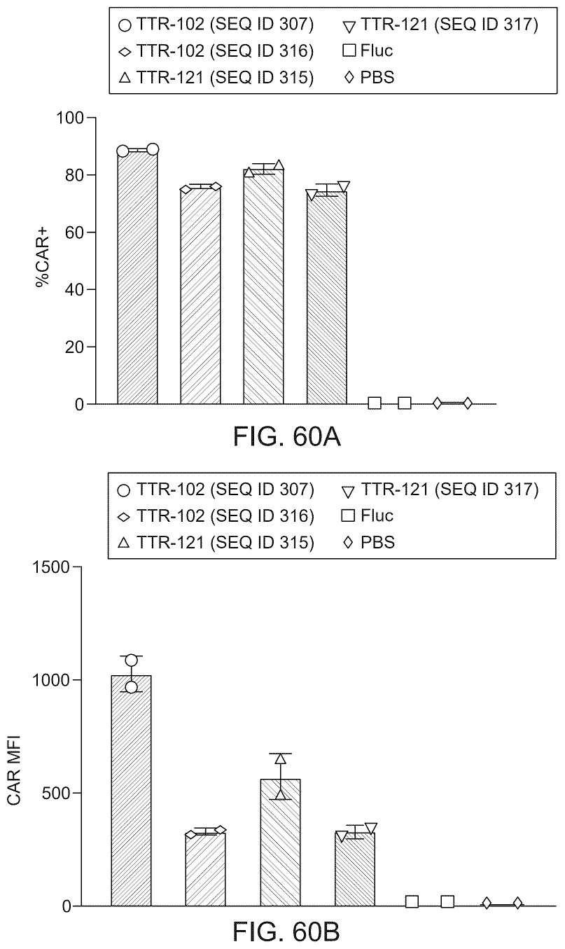

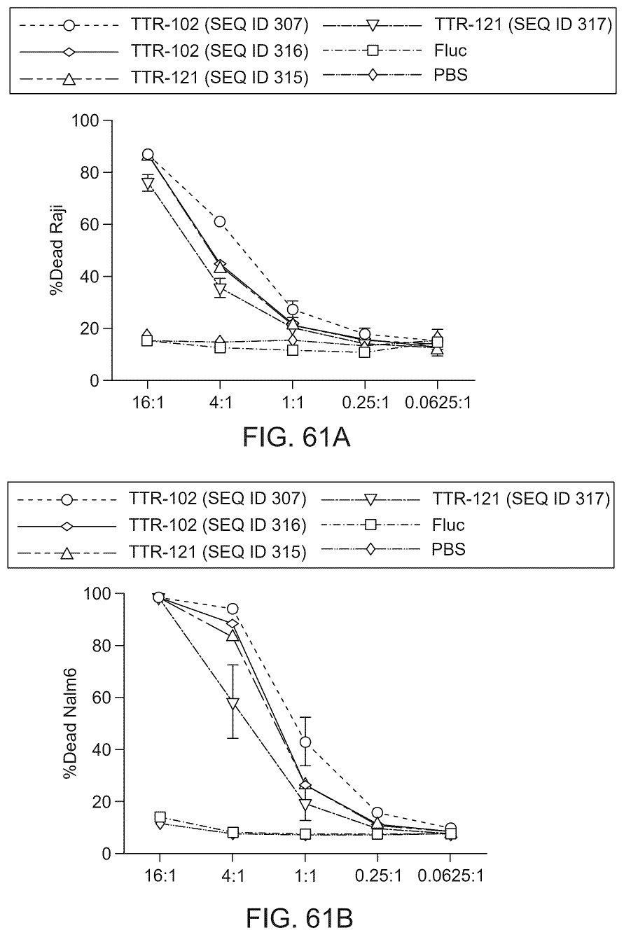

This example compares CD22 CAR-mediated cytotoxicity of Raji and Nalm6 target cells resulting from co-culture with CD8+ T-cells after transfection with Lipid 15 LNPs inserted with a CD8-targeting moiety (15C01 (SEQ ID 1)). Nanoparticles bearing mRNA encoding various UTR- and codon-optimized variants of CD22 CARs (TTR-102 and TTR-121) were produced using the microfluidic mixing and buffer exchange processes described in Example 45. An αCD8 VHH-conjugate was incorporated into the parent LNPs to obtain the final VHH-targeted LNP formulation using the process described in Example 48. Particles thus produced were tested in vitro in primary human CD8+ T cells co-cultured with Raji or Nalm6 cells to assess CAR-mediated cytotoxicity as described in Example 56. An increase in CAR-mediated cytotoxicity was observed for TTR-102 and TTR-121 containing Tidal UTRs (SEQ ID 139 and SEQ ID 146) over CoE UTRs (SEQ ID 147 and SEQ ID 148) at an effector-to-target ratio (E:T) of 4:1 for both Raji cells (FIG. 61A) and Nalm6 cells (FIG. 61B).

### Example 92. Car Expression In Vivo Using Lipid 15 LNPs Containing aCD8 (15C01 (SEQ ID 1)) Targeting Moiety and Encapsulating mRNA Encoding aCD22 CAR (TTR-102 (SEQ ID 139))

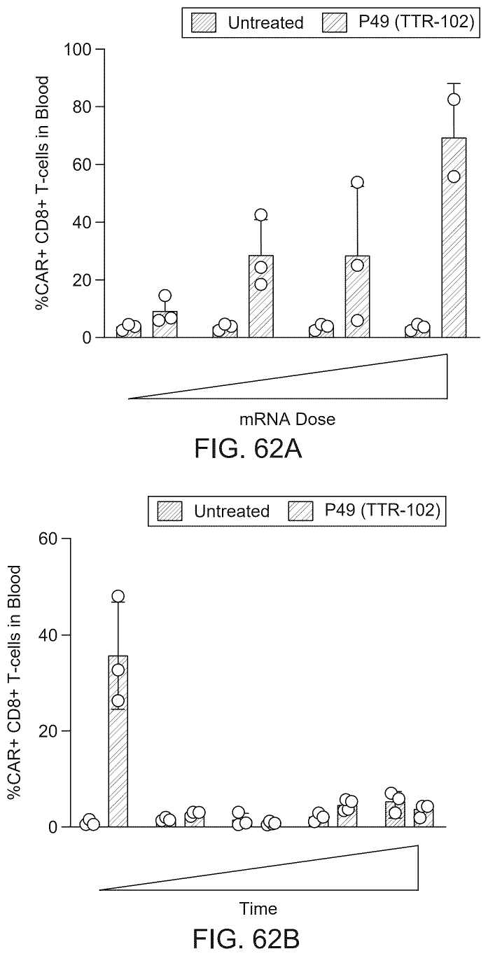

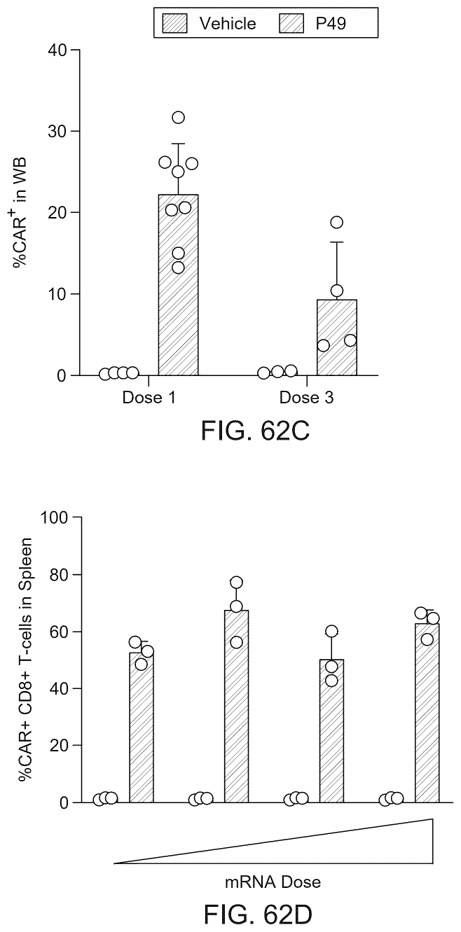

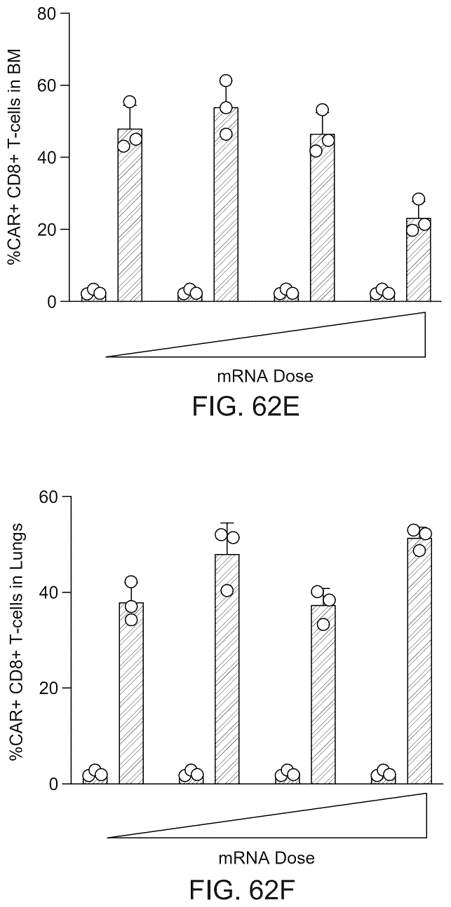

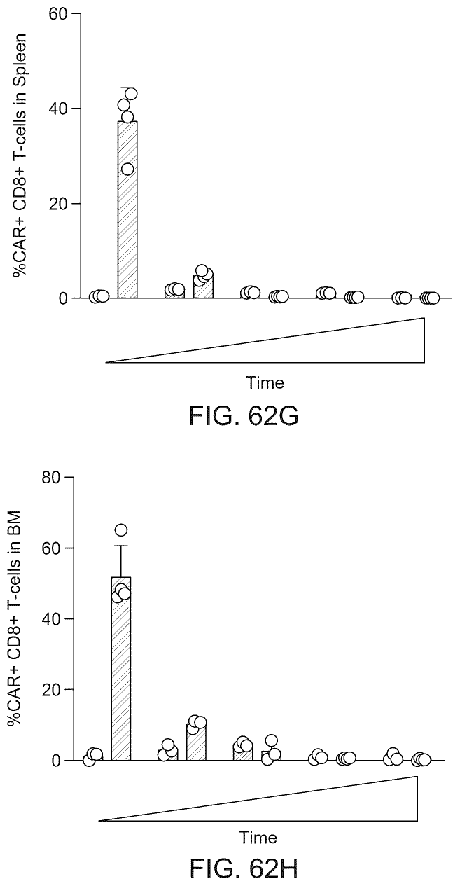

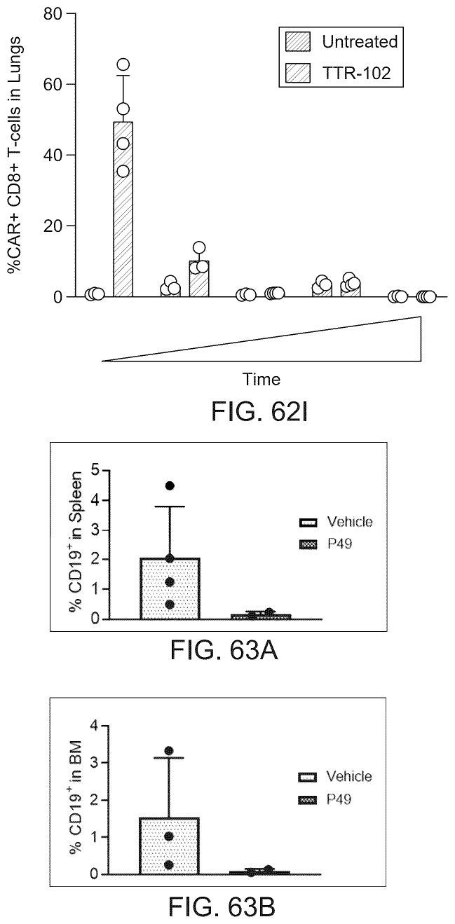

This example shows that CD8 (15C01 (SEQ ID 1))-targeted LNP deliver selectively CD22 CAR mRNA (TTR-102 (SEQ ID 139)) into CD8 T cells in vivo where they re-program T cells to transient anti-CD22 CAR T cells. PBMC-engrafted NSG mice were treated with one single injection of LNP/mRNA via tail vein at Day 14 post PBMC engraftment. First studies tested multiple dose levels of LNP/mRNA in vivo and CAR T expression in CD8 T cell was evaluated by flow cytometry 24 hour post dosing. LNP/mRNA was well tolerated at all doses tested. 40-60% CAR T expression in blood (FIG. 62A) and tissues (Lung (FIG. 62F), spleen (FIG. 62D) and bone marrow (FIG. 62E)) was achieved. Next, the persistence of CAR T in blood and tissues after one single dose of LNP/mRNA was evaluated by flow cytometry. As expected, a drop in CAR T expression was observed at the second tested time point post administration of one single dose of LNP/mRNA in blood (FIG. 62B) and tissues (FIG. 62G-FIG. 62I). CAR T detection by flow cytometry in WB or tissues was lost at the third time point post dosing. Finally, CAR T expression to repeated dosing was evaluated. LNP/RNA was well tolerated and robust CAR T expression was detected in Blood and tissues 24 hr post administration of Dose #3 (FIG. 62C).

### Example 93. B-Cell Aplasia In Vivo Using Lipid 15 LNPs Containing aCD8 (15C01 (SEQ ID 1)) Targeting Moiety and Encapsulating mRNA Encoding aCD22 CAR (TTR-102 (SEQ ID 139))

This example shows that CD8 (15C01 (SEQ ID 1))-targeted LNP deliver selectively CD22 CAR mRNA (TTR-102 (SEQ ID 139)) into CD8 T cells in vivo where they re-program T cells to functional anti-CD22 CAR T cells. B cell aplasia was used as a tool to measure functional anti-CD22 CAR T persistence. B cell aplasia was defined as <1% CD19+ B cells in Spleen and Bone Marrow of PBMC-engrafted NSG mice and determined by flow cytometry. B cell aplasia was achieved 24 hr post one single dose of LNP/RNA and persisted up to 92 hr. B cell aplasia was detected to repeated dosing 24 hr post administration of Dose #4.

### Example 94. dsRNA-Mediated Immunogenicity with MIMIC Assay

This example compares the cytokine and chemokine responses of LNPs made with different levels of double-stranded RNA (dsRNA). TTR-102 mRNA (SEQ ID 147) was purified by HPLC and then mixed with unpurified mRNA to produce mRNA with 0.2%, 0.1%, 0.04%, and 0.02% dsRNA. The HPLC purified mRNA is below the quantification level of the assay and was also used. LNPs were formulated as described in Example 45 without the addition of the CD8 targeting moiety.

A MIMIC® CRA (Cytokine release assay) was used to evaluate cytokine secretion induced by various formulations of CAR LNP constructs. MIMIC® CRA is a 3D endothelial cell-based assay that utilizes fresh human whole blood donations. Red blood cell (RBC)-depleted leucocytes along with autologous plasma are used in this assay. The assay is designed for analysis of acute immune responses generated by intravenously delivered drugs. This system has been employed in the past to retrospectively evaluate the cytokine profiles induced by TGN1412 (Dhir et al. 2012) and to various T cell engager (TCE) molecules under development (Bonnevaux et al. 2021; Abrams et al. 2022) and recently to evaluate CD19 CAR T cells.

Fresh leukocytes (RBC-depleted whole blood cells) were acquired from blood donations from normal healthy donors who provide informed consent and were enrolled in the Sanofi Pasteur VaxDesign donor program (Chesapeake Research Review, Inc., Columbia, MD., protocol 0906009). The blood components were isolated according to an established SOP. Briefly, from each volunteer's sample, whole blood leukocytes and their corresponding autologous platelet-poor plasma were separated by centrifugation at 2100 rpm. The separated plasma was spun down and cleared through a 0.2-μm filter (polyetherslfone; Nalgene, Rochester, NY) to remove platelets and platelet particles. To remove red blood cells, the leukocyte pellets were mixed with a sterile solution of 5% weight by volume (w/v) dextran (Sigma, St Louis, MO) to allow sedimentation of the erythrocytes. Thereafter, supernatants were collected and washed two times with Dulbecco's phosphate buffer saline (DPBS; Lonza). Then, white cells were counted using trypan blue exclusion staining.

To initiate the MIMIC® CRA assay, 96-well plates were coated with a collagen solution (Advanced BioMatrix, Inc., Catalog number 5005-100ML) to form a thick cushion on the bottom of the well (day 0). An endothelial cell line (EA. hy926) (ATCC, Catalog number CRL-2922) was seeded onto the collagen cushions in a media with serum one day later (day 1). After growth until confluency (about four days), the media was changed to a non-serum media. Next day (day 5) fresh reconstituted leukocytes (prepared as described above) from healthy donors were applied to the constructs along with different Tidal CAR LNPs (Table 1 and 2) diluted in autologous plasma at the dose of 10 and 1 μg/ml. CD28SA (Ancell corporation, Cat #177-820), Poly IC (Invivogen, Cat #tlrl-pic), R848 (Invivogen, Cat #vac-r848) were used as a positive control at a dose of 10 μg/ml. After 20-22 hours, culture supernatants were collected and analyzed for cytokine/chemokine production using multiplex arrays.

Culture supernatants from MIMIC® CRA were harvested after 20-22 hours of treatment with test articles and controls. Supernatants were analyzed using EMD Millipore's MILLIPLEX® MAP Human Cytokine/Chemokine Magnetic Bead Panel. This kit was used for the quantification of the following human cytokines and chemokines GM-CSF, IFNa1, IFNa2, IFNg, IL-10, IL-12p40, IL-12p70, IL-1b, IL-2, IL-4, IL-5, IL-6, IL-8, IP-10, MCP-1, MIP-1b, RANTES, TNFa. The manufacturer's protocol was followed, as prescribed, except that the standard was diluted at 1:3 instead of 1:5. Analyte concentrations were calculated based on relevant standard curves using the Bio-Plex manager software version 6.2. For run acceptance criteria, lower limit of quantification (LLOQ) and upper limit of quantification (ULOQ) for each analyte was established based on the percent recovery (Observed/Expected*100) of each point against the 5-parameter logistic curve fit of the standard values. A recovery range of 80-120% was considered acceptable, such that values falling within this range defined the lower and upper bounds of the standard curve. The raw data file was reviewed for bead counts; a data point was considered valid when a minimum of 35 beads were counted per region.

All data was exported into excel databases. Out-of-range high values (values greater than the highest point of the curve) were replaced by the highest point on the standard curve. Out-of-range low values were replaced with ½ the LLOQ.

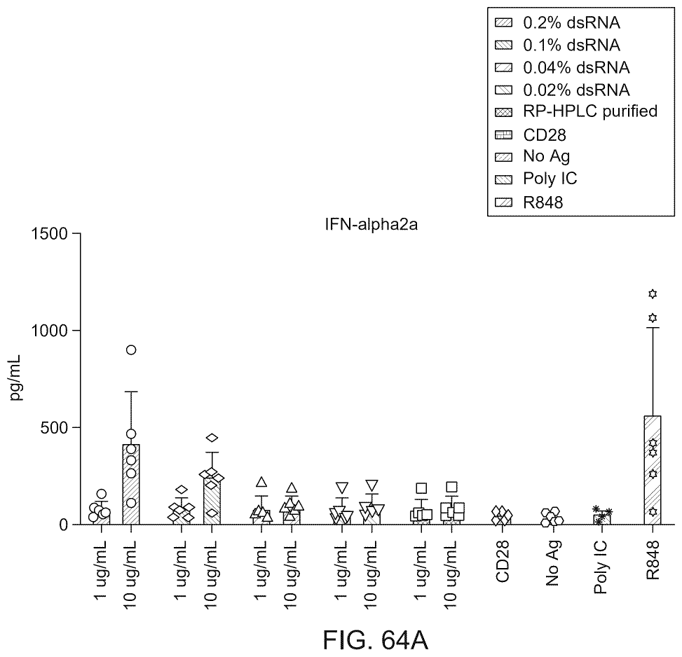

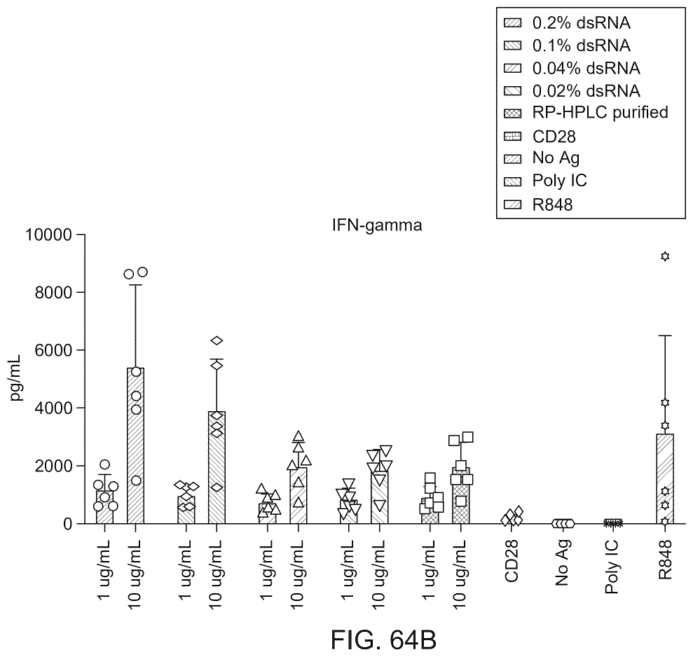

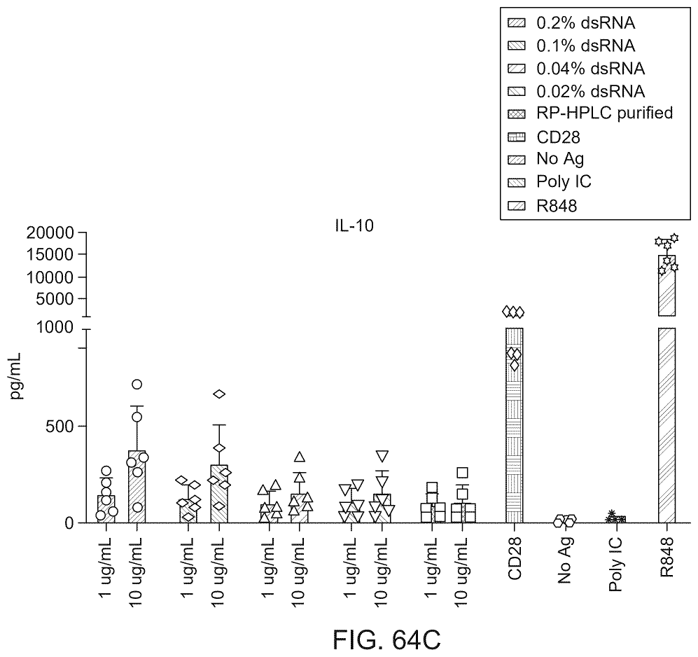

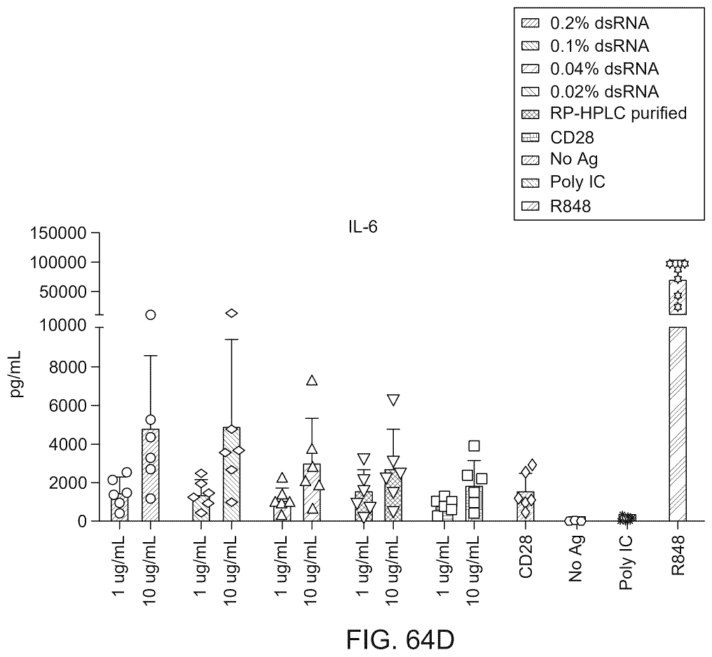

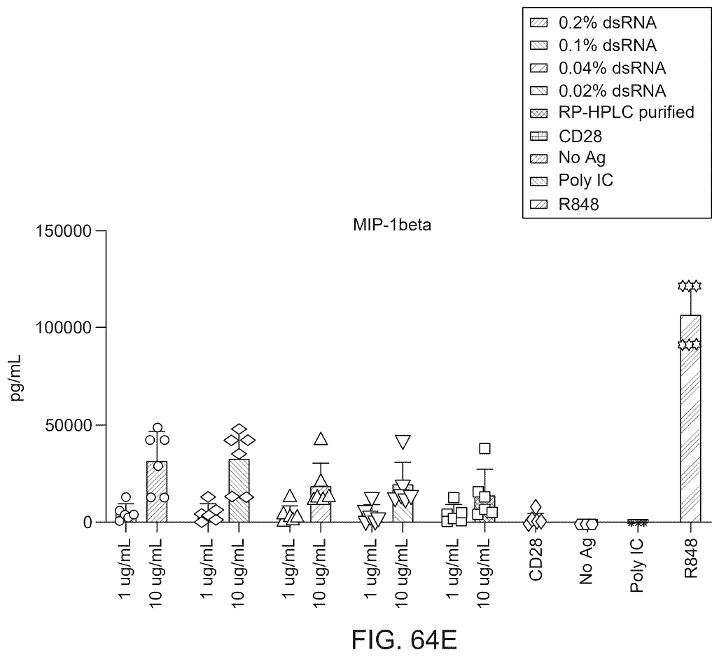

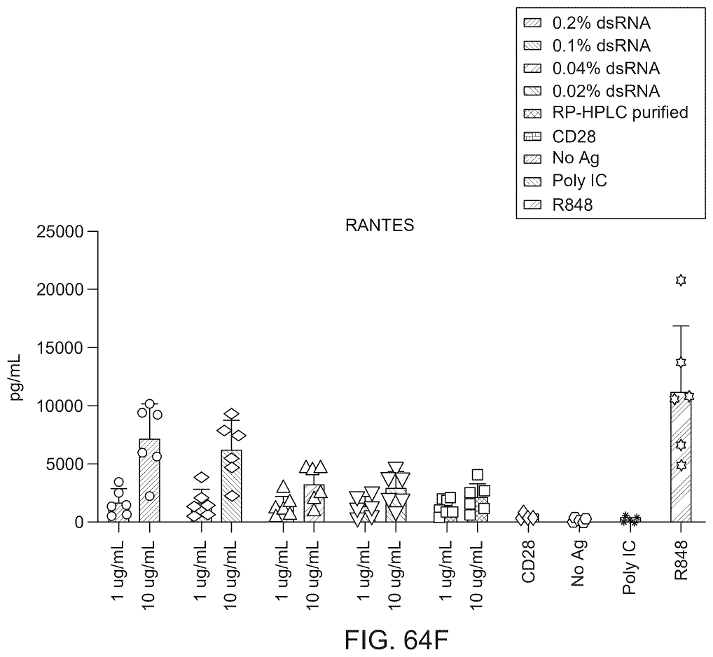

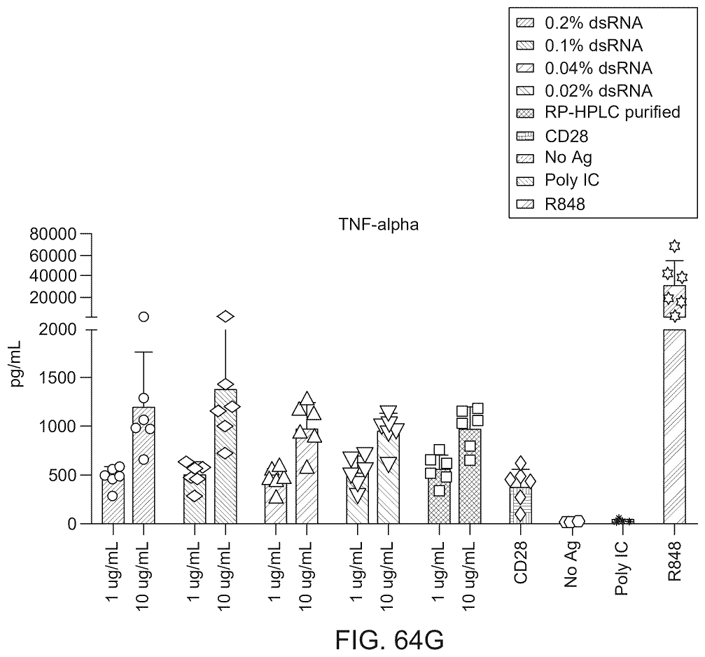

A clear cytokine signal is observed with increasing amounts of dsRNA in the LNP formulation. The increasing cytokine signal is observed for IFN-α2 (FIG. 64), which is a part of the dsRNA sensing pathway. Increases are also observed in IFN-gamma (FIG. 64B), IL-10 (FIG. 64 C), IL-6 (FIG. 64D), MIP-1beta (FIG. 64E), RANTES (FIG. 64 F), and TNF-alpha (FIG. 64G). Cytokine levels generally returned to background at levels of dsRNA below 0.04%. This trend in cytokine production was observed at both 1 and 10 μg/mL dose levels.

### Example 95. Targeted and Nontargeted LNP Comparison with MIMIC Assay Using LNPs Based on Lipid 15 Containing CD22 CAR mRNA TTR-102 (SEQ ID 139) with aCD8-Targeting Moiety (15C01V8 (SEQ ID 9))

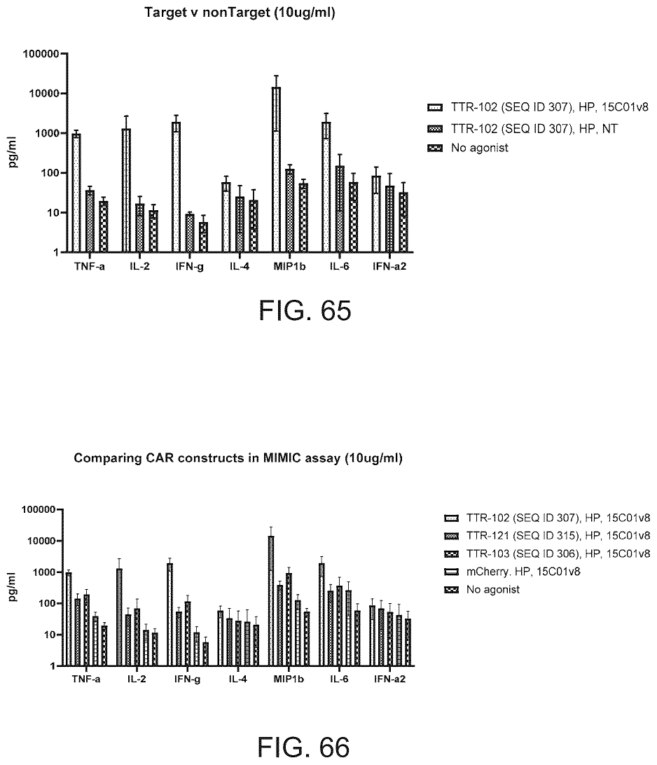

This example compares the cytokine and chemokine response of an LNP formulation based on Lipid 15 containing mRNA TTR-102 (Tidal UTRs (SEQ ID 139)) with a CD8-targeting moiety (15C01v8 (SEQ ID 9)) and an otherwise identical formulation without the CD8-targeting moiety. LNP formulations were produced as described in Example 45. The single difference between the two groups is that the targeted formulation was then post-inserted with the &CD8 V_{HH} (15C01v8)-conjugate as described in Example 48. Both formulations were then run using a MIMIC assay as described in Example 94 and dosed with 6 different donors. An increase in cytokine and chemokine induction was observed in the targeted LNP group. This includes TNF-α, IL-2, IFN-g, MIP-1b, and IL-6 (FIG. 65). The LNPs without targeting were similar to no agonist controls.

TABLE 51
Fold over background from MIMIC analysis for LNPs
with and without a CD8 targeting moiety. Presented
as mean of 6 replicates with standard deviation.

| | TTR-102 (MRT13270) (Seq ID 139), | | TTR-102 (MRT13270) | |
|---|---|---|---|---|
| | HP, 15C01v8 (SEQ ID 9) | | (Seq ID 139), HP, NT | |
| | Ave | SD | Ave | SD |
| TNF-a | 49.5 | 10.4 | 1.9 | 0.5 |
| IL-2 | 113.6 | 119.3 | 1.5 | 0.7 |
| IFN-g | 332.7 | 147.6 | 1.6 | 0.2 |
| IL-4 | 2.8 | 1.1 | 1.2 | 1.1 |
| MIP1b | 263.5 | 242.9 | 2.3 | 0.6 |
| IL-6 | 32.8 | 20.5 | 2.6 | 2.4 |
| IFN-a2 | 2.6 | 1.7 | 1.5 | 1.5 |

### Example 96. CAR Construct Comparison with MIMIC Assay Using LNPs Based on Lipid 15 Containing CD22 Car mRNA (SEQ ID 139, SEQ ID 146, SEQ ID 140) with aCD8-Targeting Moiety (15C01V8 (SEQ ID 9))

This example compares the cytokine and chemokine response of LNP formulations containing different CAR mRNAs. The LNPs were formulated as described in Example 45 with a CD8-targeting moiety (15C01v8 (SEQ ID 9) included. The mRNAs compared in this study are TTR-102 (MRT13270 (SEQ ID 139)), TTR-121 (MRT14581 (SEQ ID 146)), TTR-103 (MRT13271 (SEQ ID 140)), and mCherry. All mRNAs were purified using HPLC method before use. Formulations were compared using a MIMIC assay as described in Example 94 and dosed with 6 different donors. TTR-102 (MRT13270 (SEQ ID 139)) showed increased cytokine and chemokine expression in comparison to other tested CAR mRNAs and controls (TNF-α, IL-2, IFN-g, MIP1b, and IL-6) (FIG. 66). TTR-121 (SEQ ID 146) and TTR-103 (SEQ ID 140) were generally similar to each other and greater than mCherry for TNF-α, IL-2, IFN-g, and MIP1b.

TABLE 52
Fold over background from MIMIC analysis for multiple CAR mRNAs delivered via
targeted LNPs. Presented as mean of 6 replicates with standard deviation.

| | TTR-102 (MRT13270) | | TTR-121 (MRT14581) | | TTR-103 (MRT13271) | | mCherry. HP, | |
|---|---|---|---|---|---|---|---|---|
| | (SEQ ID 139), HP, | | (SEQ ID 146), HP, | | (SEQ ID 140), HP, | | 15C01v8 | |
| | 15C01v8 (SEQ ID 9) | | 15C01v8 (SEQ ID 9) | | 15C01v8 (SEQ ID 9) | | (SEQ ID 9) | |
| | Ave | SD | Ave | SD | Ave | SD | Ave | SD |
| TNF-a | 49.5 | 10.4 | 7.3 | 3.0 | 9.8 | 4.5 | 2.0 | 0.7 |
| IL-2 | 113.6 | 119.3 | 3.9 | 2.3 | 6.0 | 6.0 | 1.2 | 0.7 |
| IFN-g | 332.7 | 147.6 | 9.5 | 3.3 | 20.3 | 10.7 | 2.0 | 1.1 |
| IL-4 | 2.8 | 1.1 | 1.6 | 1.7 | 1.3 | 1.4 | 1.3 | 1.7 |
| MIP1b | 263.5 | 242.9 | 7.1 | 2.3 | 17.3 | 8.7 | 2.3 | 1.2 |
| IL-6 | 32.8 | 20.5 | 4.3 | 2.5 | 6.3 | 5.3 | 4.5 | 3.8 |
| IFN-a2 | 2.6 | 1.7 | 2.1 | 1.7 | 1.6 | 1.4 | 1.3 | 1.6 |

### Example 97. Lipid Type Comparison with MIMIC Assay Using LNPs Based on Lipid 15, Lipid 3, KC3, or MC3 Containing CD22 Car mRNA (SEQ ID 139)

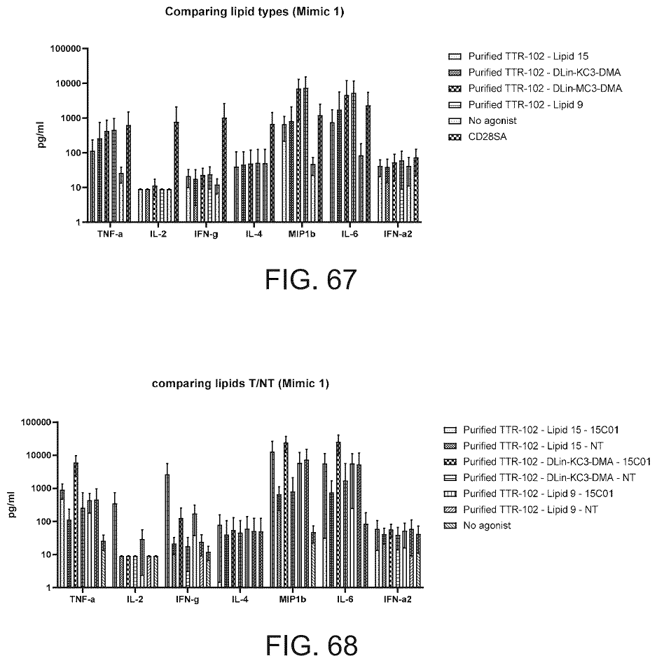

This example compares the cytokine and chemokine response of LNP formulations containing different ionizable lipids encapsulating a CD22 CAR payload (TTR-102 (SEQ ID 139)). The LNPs were formulated as described in Example 45 with HPLC purified TTR-102 mRNA (SEQ ID 139). Multiple ionizable lipids were compared in this study, including Lipid 15, KC3, MC3, and Lipid 9. A CD8-targeting moiety was not included in the test groups. Formulations were compared using a MIMIC assay as described in Example 94 and dosed at 10 μg/ml with 6 different donors. The amount of chemokines and cytokines produced by untargeted LNPs varied by ionizable lipid type. Generally, Lipid 15 showed the lowest levels of cytokine and chemokine production in comparison to KC3, MC3, and Lipid 9 (FIG. 67). This difference was observed for TNF-α, MIP1b, and IL-6. For other cytokine, IL-2, IL-4, and IFN-α2, all LNPs performed similar to no agonist controls.

### Example 98. Lipid Type Comparison with MIMIC Assay Using LNPs Based on Lipid 15, Lipid 9, KC3, or MC3 Containing CD22 Car mRNA (SEQ ID 139) with aCD8-Targeting Moiety (15C01 (SEQ ID 1))

This example compares the cytokine and chemokine response of LNP formulations containing different ionizable lipids encapsulating a CD22 CAR payload (TTR-102 (SEQ ID 139)), with and without a CD8-targeting moiety (15C01 (SEQ ID 1)). The LNPs were formulated as described in Example 45 with HPLC purified TTR-102 mRNA (SEQ ID 139). Multiple ionizable lipids were compared in this study, including Lipid 15, KC3, and Lipid 9. Formulations were compared using a MIMIC assay as described in Example 94 and dosed with 6 different donors. As shown in Example 95, targeting increases the ability of Lipid 15 formulations to induce the production of cytokines and chemokines (FIG. 68). A similar profile is observed for LNPs formulated with KC3. Lipid 9 however, showed higher levels of cytokine production from nontargeted LNPS, and the difference between targeted and nontargeted LNPS is smaller.

### Example 99: Generation of Anti-CD8 ISVD Molecules

### Generation of Recombinant Soluble CD8 Proteins

A DNA fragment encoding a secretion signal, the ectodomains of either human or cynomolgous CD8α (CD8alpha) or CD8b (CD8beta), a human IgG1 Fc domain and a HIS tag or Twin-streptag was cloned into a vector suitable for expression in CHOEBNALT85 cells (Icosagen). CHOEBNALT85 cells were transfected with equimolar quantities of both vectors. Both CD8α-Fc homodimers and CD8α/b-Fc heterodimers were purified from the culture broth 7 days after transfection by HIS trap chromatography capturing both CD8α homodimers and CD8α/b heterodimers, followed by Streptactin chromatography, separating CD8α homodimers from CD8α/b heterodimers.

Table 53 depicts the protein sequence of human and cyno CD8α-Fc-HIS6 and CD8b-Fc-Twinstrep chains.

TABLE 53
Human and cyno CD8 sequences for immunization

| | Amino acid sequence |
|---|---|
| Human CD8a-Fc-HIS6 | SQFRVSPLDRTWNLGETVELKCQVLLSNPTSGCSWLFQPRGAAA |
| | SPTFLLYLSQNKPKAAEGLDTQRFSGKRLGDTFVLTLSDFRRENE |
| | GYYFCSALSNSIMYFSHFVPVFLPAKPTTTPAPRPPTPAPTIASQP |
| | LSLRPEACRPAAGGAVHTRGLDFACPPCPAPELLGGPSVFLFPPKP |
| | KDTLMISRTPEVTCVVVDVSHEDPEVKFNWYVDGVEVHNAKTKPR |
| | EEQYNSTYRVVSVLTVLHQDWLNGKEYKCKVSNKALPAPIEKTISK |
| | AKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEA |
| | LHNHYTQKSLSLSPGHHHHHH |
| Human CD8b-Fc-Twinstrep | LQQTPAYIKVQTNKMVMLSCEAKISLSNMRIYWLRQRQAPSSDS |
| | HHEFLALWDSAKGTIHGEEVEQEKIAVFRDASRFILNLTSVKPED |
| | SGIYFCMIVGSPELTFGKGTQLSVVDFLPTTAQPTKKSTLKKRVC |
| | RLPRPETQKGPLCPPCPAPELLGGPSVFLFPPKPKDTLMISRTPEVTC |
| | VVVDVSHEDPEVKFNWYVDGVEVHNAKTKPREEQYNSTYRVVSVL |
| | TVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| | WSHPQFEKGGGSGGGSGGSSAWSHPQFEK |
| Cynomolgous CD8a-Fc-HIS6 | NQFRVSPLGRTWNLGETVELKCQVLLSNPTSGCSWLFQPRGTA |
| | ARPTFLLYLSQNKPKAAEGLDTQRFSGKRLGDTFVLTLRDFRQE |
| | NEGYYFCSALSNSIMYFSHFVPVFLPAKPTTTPAPRPPTPAPTTAS |
| | QPLSLRPEACRPAAGGSVNTRGLDFACPPCPAPELLGGPSVFLFPP |
| | KPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHE |
| | ALHNHYTQKSLSLSPGHHHHHH |
| Cynomolgous CD8b-Fc- | LQQTPAYIQVQTNKMAMLSCEAKISLGNMRIYWLRQRQAPSSDS |
| Twinstrep | HHEFLALWDSTKGTVHGEEVAQEKIAVFRDASRFILNLTSVKPE |
| | DSGIYFCMIIGSPELTFGKGTQLSVVDFLPTTAQPTKKSTPKKRG |
| | CRLPRPATQKGPLCPPCPAPELLGGPSVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPIEKTISKAKGQPREPQVYTL |
| | PPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLS |
| | PGWSHPQFEKGGGSGGGSGGSSAWSHPQFEK |

### Immunizations

To induce a heavy-chain antibody (HcAb)-dependent humoral immune response, two llamas and one alpaca were immunized four times with DNA encoding full-length recombinant human CD8α and CD8b.

### Library Constructions and Phage Display

Immune blood samples were taken at regular intervals, serum responses were determined, and total RNA was prepared from the isolated PBL. From these blood samples, peripheral blood mononuclear cells (PBMCs) were prepared using Ficoll-Hypaque according to the manufacturer's instructions (Amersham Biosciences, Piscataway, NJ, US). From the PBMCs, total RNA was extracted and used as starting material for RT-PCR to amplify the ISVD-encoding DNA segments, essentially as described in WO 2005/044858. Subsequently, phages were prepared according to standard protocols (see for example the prior art and applications filed by Ablynx N.V. cited herein) and stored after filter sterilization at 4° C. for further use.

For general phage display selection techniques, reference is made to Antibody Phage Display: Methods and Protocols (First Edition, 2002, O'Brian and Aitken eds., Humana Press, Totowa, NJ). Two rounds of panning phage display selections were performed using the llama and alpaca ISVD immune libraries in alternating selection rounds on human and cynomolgus CD8α-Fc homodimers or CD8α/b heterodimers. The output from the selection was plated onto LB/amp/2% glu plates. Colonies were picked and grown in 96 deep well plates (1 ml volume) and induced by adding IPTG for ISVD expression. Periplasmic extracts (volume: ~80 μl) were prepared according to standard methods (see for example the prior art and applications filed by applicant cited herein).

### Primary Screening and Sequencing

Primary screening of periplasmic extracts was performed by a bead based multiplex screening assay (xMAP). Human CD8a-Fc, human CD8a/b-Fc, cyno CD8a-Fc or cyno CD8a/b Fc proteins were coupled to spectrally distinct sets of Magplex beads, a mix of the respective beads was prepared and incubated with 1/10 diluted periplasmic extract and anti-FLAG-PE as a detection reagent. Microspheres coupled with huIgG were additionally included in the mix so that clones that bind to the Fc portion of the CD8-Fc proteins could be excluded.

Clones that showed binding to human and cyno CD8α-Fc, but not to huIgG were sequenced Sequence analysis of ISVDs from phage display selection outputs was done according to commonly known procedures (Pardon et al. 2014, Nat. Protoc. 9:674). 731 unique sequences were obtained.

### Example 100: Characterization of the Unique Sequences

### Off-Rate Analysis of the Unique ISVD Clones

All unique clones from the sequence analysis were subjected to off-rate analysis towards human and cyno CD8a-Fc and CD8a/b-Fc by Surface Plasmon Resonance (SPR) on a ProteOn XPR36 instrument (Bio-Rad Laboratories). The experiment was performed at 25° C. As running buffer 2×HBS-EP+ was used. Anti-hu-Fc ISVD was immobilized on 6 different ligand lanes from a GLC sensorchip (Bio-Rad Laboratories) with the ProteOn Amine Coupling Kit (Bio-Rad Laboratories) according to the manufacturer's instructions. Human CD8a-Fc, cyno CD8a-Fc, human CD8a/b-Fc and cyno CD8a/b-Fc were captured on the anti-hu-Fc ISVD immobilized ligand lanes. One ligand lane served as a reference surface and no target was captured on the anti-hu-Fc ISVD surface. Samples (periplasmic extracts containing unique ISVDs) diluted 1:10 in running buffer were flowed over for 2 minutes, followed by a constant flow of the assay buffer for 10 minutes. Between sample injections, the surfaces were regenerated with 0.85% H3PO4. Several buffer blanks were injected for double referencing.

Data was analyzed with the ProteOn Manager 3.1.0 software (Bio-Rad Laboratories), off-rates were determined based on the 1:1 interaction model (Langmuir binding model). Only clones with an off rate slower than 10-2/s were retained.

### Selection of Initial Lead Panel

Based on off-rate on human and cyno CD8 and on sequence uniqueness, 43 clones were selected for expression, purification, and characterization.

### Example 101: Generation of ISVD Constructs

For the selected clones, ISVD-containing DNA fragments, obtained by PCR with specific combinations of forward FR1 and reverse FR4 primers each carrying a unique restriction site, were digested with the appropriate restriction enzymes, and ligated into the matching cloning cassettes of ISVD expression vectors (described below). The ligation mixtures were then transformed to electrocompetent Escherichia coli TG1 (60502, Lucigen, Middleton, WI) or TOP10 (C404052, ThermoFisher Scientific, Waltham, MA) cells which were then grown under the appropriate antibiotic selection pressure (kanamycin or Zeocin). Resistant clones were verified by Sanger sequencing of plasmid DNA (LGC Genomics, Berlin, Germany).

ISVDs were expressed in E. coli TG1 from a plasmid expression vector containing the lac promoter, a resistance gene for kanamycin, an E. coli replication origin and a ISVD cloning site preceded by the coding sequence for the OmpA signal peptide. In frame with the ISVD coding sequence, the vector codes for a C-terminal FLAG3 and HIS6 tag. The signal peptide directs the expressed ISVD to the periplasmic compartment of the bacterial host.

HIS6-tagged ISVDs were purified by immobilized metal affinity chromatography (IMAC) followed by a desalting step and if necessary, gel filtration chromatography (Superdex column, GE Healthcare) in PBS. Else, ISVDs were purified on Protein A followed by a desalting step and if necessary, gel filtration chromatography (Superdex column, GE Healthcare) in PBS. The purity and integrity of the ISVDs was verified by SDS-PAGE and mass spectrometry.

### Example 102: Characterization in Cell-Based Assay

The monovalent lead panel ISVDs were characterized in binding FACS.

### Binding FACS

Binding of purified anti-CD8 ISVDs to CD8⁺ T cells was evaluated by flow cytometry. A serial dilution of each ISVD starting at 1 μM was applied to human T cells (purified from peripheral blood monocytes). ISVDs were allowed to bind for 30 minutes at 4° C. in FACS buffer (PBS supplemented with 10% FBS and 0.05% azide). Cells were washed by centrifugation/resuspension in FACS buffer and bound ISVD probed with a Brilliant Violet 421 anti-Flag antibody (Biolegend; cat #: F1804) for 30 minutes at 4° C. In the same incubation step, CD4+ T cells were probed with APC anti-human CD4 [RPA-T4] (Biolegend). Cells were washed again and incubated with propidium Iodide to stain for dead cells. The cells were analysed via an iQue Plus. Dead cells and CD4+ cells were gated out from the analysis (mean fluorescence intensity in function of ISVD concentration).

For 22 of the 43 ISVDs, a dose-response curve could be fitted and an EC50 calculated (Table below).

TABLE 54
FACS binding analysis of purified ISVDs on human CD8+ T cells.

| | FACS (EC50) (M) |
|---|---|
| ID | CD8⁺ T cells |
| T0347013F01 | 1.5E−10 |
| T0347014D02 | 1.8E−10 |
| T0347014G05 | 1.9E−10 |
| T0347015C01 | 2.0E−10 |
| T0347023A12 | 4.2E−10 |
| T0347002C04 | 4.6E−10 |
| T0347022A02 | 4.9E−10 |
| T0347014D12 | 8.4E−10 |
| T0347014D12 | 1.0E−09 |
| T0347001B04 | 1.4E−09 |
| T0347001E05 | 2.0E−09 |
| T0347001B02 | 2.3E−09 |
| T0347021B10 | 2.5E−09 |
| T0347013B01 | 2.6E−09 |
| T0347002C07 | 4.3E−09 |
| T0347002A07 | 7.3E−09 |
| T0347014A02 | 8.3E−09 |
| T0347012C08 | 8.5E−09 |
| T0347001D12 | 1.1E−08 |
| T0347013G07 | 1.4E−08 |
| T0347008C01 | 2.6E−08 |
| T0347008A10 | 2.8E−08 |

### Functional Testing of Monovalent ISVDs in T Cell Transfection Assays

See above Examples.

The functional assay allowed a clear ranking of the initial lead panel. Clones from different families that had reasonable potency in the TT assay and FACS were selected. This led to a filtered lead panel of 4 ISVDs.

### Affinity Determination of Monovalent ISVDs of Filtered Lead Panel

The affinity (KD) of the lead panel clones to human and cyno CD8α-Fc was determined via SPR on a Biacore 8K⁺ (Cytiva). The experiment was performed at 25° C., as running buffer HBS-EP+1× (Cytiva, cat #BR100669) was used. An anti-hIgG (Fc) ISVD was immobilized on a Biacore CM5 chip by amine coupling according to the manufacturer's instructions. Human and cynomolgus CD8α-Fc were captured via their Fc-portion at a concentration of 1.25 μg/ml, and then nine different concentrations of the clones (diluted in assay buffer) were applied. Samples were applied to the respective targets in multi-cycle kinetics for 2 minutes, followed by a constant flow of the running buffer for 15 minutes. Between the different injections, the surfaces were regenerated with 0.85% H₃PO₄. Data was analysed with the Biacore Insight Evaluation software V5.0.18.22102 (Cytiva). The kinetic properties (ka and kd) were determined using the Langmuir 1:1 model. The equilibrium dissociation constant KD was calculated as the kd/ka ratio. Results are shown in Table 55.

TABLE 55
Kinetic analysis of lead panel ISVDs
on human and cynomolgous CD8a-Fc

| | | Ka | Kd | KD |
|---|---|---|---|---|
| ID | Target | 1/Ms | 1/s | M |
| T0347015C01 | Human CD8a-Fc | 2.27E+06 | 3.01E−04 | 1.33E−10 |
| (SEQ ID | Cynomolgous | 2.58E+06 | 2.72E−04 | 1.06E−10 |
| NO. 1) | CD8a-Fc | | | |
| T0347001E05 | Human CD8a-Fc | 4.79E+06 | 4.53E−04 | 9.47E−11 |
| | Cynomolgous | 2.66E+05 | 1.18E−03 | 4.45E−09 |
| | CD8a-Fc | | | |
| T0347002A07 | Human CD8a-Fc | 2.82E+06 | 2.73E−02 | 9.67E−09 |
| | Cynomolgous | 9.43E+04 | 2.02E−02 | 2.14E−07 |
| | CD8a-Fc | | | |
| T0347013G07 | Human CD8a-Fc | 2.07E+06 | 2.52E−02 | 1.22E−08 |
| | Cynomolgous | 5.72E+04 | 1.05E−04 | 1.84E−09 |
| | CD8a-Fc | | | |

Based on the affinity determination and on all other characterization data, ISVD T0347015C01 (SEQ ID NO. 1) was selected for further optimization.

### Example 103: Sequence Optimization of Isvd T0347015C01

The protein sequence of anti-CD8 ISVD T0347015C01 was further optimized, involving the mutagenesis of one or more specific amino acid residues to make the ISVD more human like, to reduce its binding by pre-existing antibodies and to improve its long-term stability. Nine sequence variants of ISVD T0347015C01 (SEQ ID Nos. 1 to 9) showed preserved binding (ka, kd and KD) to human and cynomolgous CD8α-Fc (Table 56). their melting temperature (TSA) was determined, and their functional properties as part of a targeted liponanoparticle were assessed.

TABLE 56
Kinetic analysis of ISVD T0347015C01 variants
on human and cynomolgous CD8a-Fc

| | | Ka | Kd | KD | Tm |
|---|---|---|---|---|---|
| ID | Target | 1/Ms | 1/s | M | ° C. |
| A044300805 | Human CD8a-Fc | 2.11E+06 | 4.97E−04 | 2.35E−10 | |
| (SEQ ID NO: 1) | Cynomolgous CD8a-Fc | 1.77E+06 | 5.37E−04 | 3.03E−10 | 74 |
| A044300805_v1 | Human CD8a-Fc | 1.18E+06 | 6.06E−04 | 5.12E−10 | |
| (SEQ ID NO: 2) | Cynomolgous CD8a-Fc | 1.11E+06 | 6.89E−04 | 6.20E−10 | 73 |
| A044300805_v2 | Human CD8a-Fc | 2.41E+06 | 5.54E−04 | 2.30E−10 | |
| (SEQ ID NO: 3) | Cynomolgous CD8a-Fc | 1.88E+06 | 5.64E−04 | 3.00E−10 | 74 |
| A044300805_v3 | Human CD8a-Fc | 2.69E+06 | 5.09E−04 | 1.89E−10 | |
| (SEQ ID NO: 4) | Cynomolgous CD8a-Fc | 1.84E+06 | 4.98E−04 | 2.70E−10 | 70 |
| A044300805_v4 | Human CD8a-Fc | 1.34E+06 | 5.21E−04 | 3.89E−10 | |
| (SEQ ID NO: 5) | Cynomolgous CD8a-Fc | 1.22E+06 | 6.22E−04 | 5.09E−10 | 69 |
| A044300805_v5 | Human CD8a-Fc | 3.38E+06 | 5.99E−04 | 1.77E−10 | |
| (SEQ ID NO: 6) | Cynomolgous CDSa-Fc | 2.48E+06 | 5.96E−04 | 2.40E−10 | 70 |
| A044300805_v6 | Human CD8a-Fc | 1.93E+06 | 4.87E−04 | 2.52E−10 | |
| (SEQ ID NO: 7) | Cynomolgous CD8a-Fc | 1.63E+06 | 4.90E−04 | 3.02E−10 | 74 |
| A044300805_v7 | Human CD8a-Fc | 1.51E+06 | 5.97E−04 | 3.95E−10 | |
| (SEQ ID NO: 8) | Cynomolgous CD8a-Fc | 1.29E+06 | 6.47E−04 | 5.00E−10 | 68 |
| A044300805_v8 | Human CD8a-Fc | 2.77E+06 | 5.35E−04 | 1.93E−10 | |
| (SEQ ID NO: 9) | Cynomolgous CD8a-Fc | 3.71E+06 | 7.51E−04 | 2.02E−10 | 69 |

TABLE 57
T0347015C01 and variants sequences

| | | SEQ | SEQ | SEQ | SEQ | SEQ | SEQ | SEQ | SEQ |
|---|---|---|---|---|---|---|---|---|---|
| | | ID (full | ID | ID | ID | ID | ID | ID | ID |
| name | Description (modifications compared to T0347015C01) | sequence) | (fw1) | (CDR1) | (fw2) | (CDR2) | (fw3) | (CDR3) | (fw4) |
| Abm CDR | | | | | | | | | |
| definition | | | | | | | | | |
| T0347015C01 | T0347015C01 | 160 | 180 | 181 | 182 | 183 | 184 | 185 | 186 |
| A044300805 | T0347015C01(L11V, A14P, D30E, G55E, N56Q, I60A, | 161 | 187 | 188 | 189 | 190 | 191 | 192 | 193 |
| | S71R, K83R, A89L, N100kG, M100oL, K105Q) | | | | | | | | |
| A044300805_v1 | T0347015C01(L11V, A14P, D30E, G55E, N56Q, I60A, | 162 | 194 | 195 | 196 | 197 | 198 | 199 | 200 |
| (T0347015C01v1; | S71R, K83R, A89L, K100bE, N100kG, M100oL, K105Q) | | | | | | | | |
| or 15C01v1) | | | | | | | | | |
| A044300805_v2 | T0347015C01(L11V, A14P, D30E, G55E, N56Q, I60A, | 163 | 201 | 202 | 203 | 204 | 205 | 206 | 207 |
| (T0347015C01v2; | S71R, K83R, A89L, K100bY, N100kG, M100oL, K105Q) | | | | | | | | |
| or 15C01v2) | | | | | | | | | |
| A044300805_v3 | T0347015C01(L11V, A14P, D30E, G55E, N56Q, I60A, | 164 | 208 | 209 | 210 | 211 | 212 | 213 | 214 |
| (T0347015C01v3; | S71R, Y79D, K83R, A89L, N100kG, M100oL, K105Q) | | | | | | | | |
| or 15C01v3) | | | | | | | | | |
| A044300805_v4 | T0347015C01(L11V, A14P, D30E, G55E, N56Q, I60A, | 165 | 215 | 216 | 217 | 218 | 219 | 220 | 221 |
| (T0347015C01v4; | S71R, Y79D, K83R, A89L, K100bE, N100kG, M100oL, | | | | | | | | |
| or 15C01v4) | K105Q) | | | | | | | | |
| A044300805_v5 | T0347015C01(L11V, A14P, D30E, G55E, N56Q, I60A, | 166 | 222 | 223 | 224 | 225 | 226 | 227 | 228 |
| (T0347015C01v5; | S71R, Y79D, K83R, A89L, K100bY, N100kG, M100oL, | | | | | | | | |
| or 15C01v5) | K105Q) | | | | | | | | |
| A044300805_v6 | T0347015C01(L11V, A14P, D30E, G55E, N56Q, I60A, | 167 | 229 | 230 | 231 | 232 | 233 | 234 | 235 |
| (T0347015C01v6; | S71R, Y79S, K83R, A89L, N100kG, M100oL, K105Q) | | | | | | | | |
| or 15C01v6) | | | | | | | | | |
| A044300805_v7 | T0347015C01(L11V, A14P, D30E, G55E, N56Q, I60A, | 168 | 236 | 237 | 238 | 239 | 240 | 241 | 242 |
| (T0347015C01v7; | S71R, Y79S, K83R, A89L, K100bE, N100kG, M100oL, | | | | | | | | |
| or 15C01v7) | K105Q) | | | | | | | | |
| A044300805_v8 | T0347015C01(L11V, A14P, D30E, G55E, N56Q, I60A, | 169 | 243 | 244 | 245 | 246 | 247 | 248 | 249 |
| (T0347015C01v8; | S71R, Y79S, K83R, A89L, K100bY, N100kG, M100oL, | | | | | | | | |
| or 15001v8) | K105Q) | | | | | | | | |
| Kabat CDR | | | | | | | | | |
| definition | | | | | | | | | |
| T0347015C01 | T0347015C01 | 170 | 250 | 251 | 252 | 253 | 254 | 255 | 256 |
| A044300805 | T0347015C01(L11V, A14P, D30E, G55E, N56Q, I60A, | 171 | 257 | 258 | 259 | 260 | 261 | 262 | 263 |
| | S71R, K83R, A89L, N100kG, M100oL, K105Q) | | | | | | | | |
| A044300805_v1 | T0347015C01(L11V, A14P, D30E, G55E, N56Q, I60A, | 172 | 264 | 265 | 266 | 267 | 268 | 269 | 270 |
| (T0347015C01v1; | S71R, K83R, A89L, K100bE, N100kG, M100oL, K105Q) | | | | | | | | |
| or 15C01v1) | | | | | | | | | |
| A044300805_v2 | T0347015C01(L11V, A14P, D30E, G55E, N56Q, I60A, | 173 | 271 | 272 | 273 | 274 | 275 | 276 | 277 |
| (T0347015C01v2; | S71R, K83R, A89L, K100bY, N100kG, M100oL, K105Q) | | | | | | | | |
| or 15C01v2) | | | | | | | | | |
| A044300805_v3 | T0347015C01(L11V, A14P, D30E, G55E, N56Q, I60A, | 174 | 278 | 279 | 280 | 281 | 282 | 283 | 284 |
| (T0347015C01v3; | S71R, Y79D, K83R, A89L, N100kG, M100oL, K105Q) | | | | | | | | |
| or 15C01v3) | | | | | | | | | |
| A044300805_v4 | T0347015C01(L11V, A14P, D30E, G55E, N56Q, I60A, | 175 | 285 | 286 | 287 | 288 | 289 | 290 | 291 |
| (T0347015C01v4; | S71R, Y79D, K83R, A89L, K100bE, N100kG, M100oL, | | | | | | | | |
| or 15C01v4) | K105Q) | | | | | | | | |
| A044300805_v5 | T0347015C01(L11V, A14P, D30E, G55E, N56Q, I60A, | 176 | 292 | 293 | 294 | 295 | 296 | 297 | 298 |
| (T0347015C01v5; | S71R, Y79D, K83R, A89L, K100bY, N100kG, M100oL, | | | | | | | | |
| or 15C01v5) | K105Q) | | | | | | | | |
| A044300805_v6 | T0347015C01(L11V, A14P, D30E, G55E, N56Q, I60A, | 177 | 299 | 300 | 301 | 302 | 303 | 304 | 305 |
| (T0347015C01v6; | S71R, Y79S, K83R, A89L, N100kG, M100oL, K105Q) | | | | | | | | |
| or 15C01v6) | | | | | | | | | |
| A044300805_v7 | T0347015C01(L11V, A14P, D30E, G55E, N56Q, I60A, | 178 | 306 | 307 | 308 | 309 | 310 | 311 | 312 |
| (T0347015001v7; | S71R, Y79S, K83R, A89L, K100bE, N100kG, M100oL, | | | | | | | | |
| or 15001v7) | K105Q) | | | | | | | | |
| A044300805_v8 | T0347015C01(L11V, A14P, D30E, G55E, N56Q, I60A, | 179 | 313 | 314 | 315 | 316 | 317 | 318 | 319 |
| (T0347015001v8; | S71R, Y79S, K83R, A89L, K100bY, N100kG, M100oL, | | | | | | | | |
| or 15C01v8) | K105Q) | | | | | | | | |

### Melting Temperature of T0347015C01 Sequence Variants

A thermal shift assay (TSA) was performed in a 96-well plate on a qPCR machine (LightCycler 480II, Roche). Per row, one ISVD protein was analysed in the following pH range: 4, 5, 6, 7, 8 and 9.

Per well, 5 μL of ISVD protein sample (0.8 mg/mL in D-PBS) was added to 5 μL of Sypro Orange (40× in MilliQ water; Invitrogen, Cat. No. S6551) and 10 μL of buffer (100 mM phosphate, 100 mM borate, 100 mM citrate and 115 mM NaCl with a pH ranging from 4 to 9). A temperature gradient (37 to 99° C. at a rate of 0.03° C./s) was applied, which induced unfolding of the ISVD proteins, and hence exposure of hydrophobic patches. Binding of Sypro Orange to those hydrophobic patches, caused increase in fluorescence intensity, which was measured (Ex/Em=465/580 nm). The inflection point of the first derivative of the fluorescence intensity curve at pH 7 served as a measure of the melting temperature (Tm).

The Tm (° C.) values obtained for the sequence optimized variants are given in Table 58.

TABLE 58
Tm of ISVD T0347015C01 sequence variants

| ID | Tm (° C.) |
|---|---|
| A044300805 | 74 |
| A044300805_v1 | 73 |
| A044300805_v2 | 74 |
| A044300805_v3 | 70 |
| A044300805_v4 | 69 |
| A044300805_v5 | 70 |
| A044300805_v6 | 74 |
| A044300805_v7 | 68 |
| A044300805_v8 | 69 |

### Example 104: Structure Determination by CryoEM of ISVD A044300805_v8 Binding to Human CD8 Alpha/Beta Dimer

For further insight into the mode of action, cryo-EM analysis was done to determine the epitope/paratope relationship of CD8alpha (>sp|P01732|CD8A_HUMAN T-cell surface glycoprotein CD8 alpha chain OS=Homo sapiens OX=9606 GN-CD8A PE=1 SV=1 MALPVTALLLPLALLLHAARPSQFRVSPLDRTWNLGETVELKCQVLLSNPTSGCSWLFQ PRGAAASPTFLLYLSQNKPKAAEGLDTQRFSGKRLGDTFVLTLSDFRRENEGYYFCSAL SNSIMYFSHFVPVFLPAKPTTTPAPRPPTPAPTIASQPLSLRPEACRPAAGGAVHTRGLDF ACDIYIWAPLAGTCGVLLLSLVITLYCNHRNRRRVCKCPRPVVKSGDKPSLSARYV (SEQ ID NO: 570) with ISVD A044300805_v8 (SEQ ID NO: 169). All structure determinations were done in combination with anti-VHH Fab38 and different in house developed Fabs (binding the VHH) to stabilize and increase the size of the complex.

### Methods

To prepare the complex for cryo-EM analysis, CD8 recombinant protein (human CD8 alpha beta R&D Systems), ISVDs and an anti-ISVD Fab were mixed at a molar ratio of 1:2:1 and incubated on ice for 1 hour to form a stable tertiary complex. The mixture was then subjected to HPLC-SEC analysis to assess purity and isolate the desired complex. The HPLC-SEC was conducted on an Agilent Infinity HPLC system equipped with a Superdex 200 Increase column (Cytiva). The mixture was injected at a concentration of approximately 5 mg/mL in a buffer containing 150 mM NaCl and 20 mM Hepes (pH 7.4). The column was operated at a flow rate of 0.05 mL/min, and protein elution was monitored by absorbance at 280 nm. The retention times were compared with molecular weight standards to identify the CD8-ISVD-Fab complex. Fractions corresponding to the complex were pooled, adjusted to ~0.5 mg/mL, and used for cryo-EM grid preparation.

Purified CD8-ISVD-Fab complexes were diluted to ~0.5 mg/mL in buffer containing 150 mM NaCl and 20 mM Hepes (pH 7.4). Aliquots (3 μL) of the sample were applied to glow-discharged Ultrafoil R^{0.6/1.0} grids (Quantifoil), blotted for 4 seconds under 100% humidity and 4° C. using a Vitrobot Mark IV (Thermo Fisher Scientific), and plunge-frozen in liquid ethane cooled by liquid nitrogen. Grids were stored in liquid nitrogen until data collection.

Cryo-EM data were collected using a Glacios cryo-transmission electron microscope (Thermo Fisher Scientific) operating at 200 kV and equipped with a Falcon 4i direct electron detector and an energy filter operated in zero-loss mode with a slit width of 10 eV. Movies were recorded in counting mode at a nominal magnification of 205,000×, corresponding to a pixel size of 0.58 Å. Each movie comprised 40 frames with a total exposure time of 2-3 seconds, yielding a cumulative dose of ~50 e⁻/Å². Automated data acquisition was performed using EPU software, targeting~8000 micrographs per dataset.

Cryo-EM data processing was performed in cryoSPARC. Raw movies were corrected for beam-induced motion using Patch Motion Correction, and contrast transfer function (CTF) estimation was performed with Patch CTF Estimation. Particle picking was conducted using template-based particle picking, and extracted particles underwent iterative rounds of 2D classification to remove noise and select high-quality particles.

Initial 3D maps were generated using Ab-Initio Reconstruction (multiple classes) to distinguish different particle orientations and conformations. Selected classes were further refined using Non-Uniform Refinement to achieve high-resolution reconstructions. Local refinement was employed to improve specific regions of interest, particularly at flexible interfaces within the complex. Final maps were evaluated for resolution using the gold-standard Fourier shell correlation (FSC) at the 0.143 cutoff.

Initial atomic models were generated using AlphaFold predictions for individual protein components and were manually fitted into the cryo-EM density maps using Coot. Manual adjustments were made iteratively to resolve ambiguities and optimize fit. Real-space refinement of the models was performed in Phenix, including minimization of atomic clashes and validation of geometry. Final models were evaluated using Phenix validation tools, including assessment of the Ramachandran plot, clash score, and overall model-to-map correlation.

Determination of the Interaction Sites of ISVD A044300805 v8 with CD8-Alpha

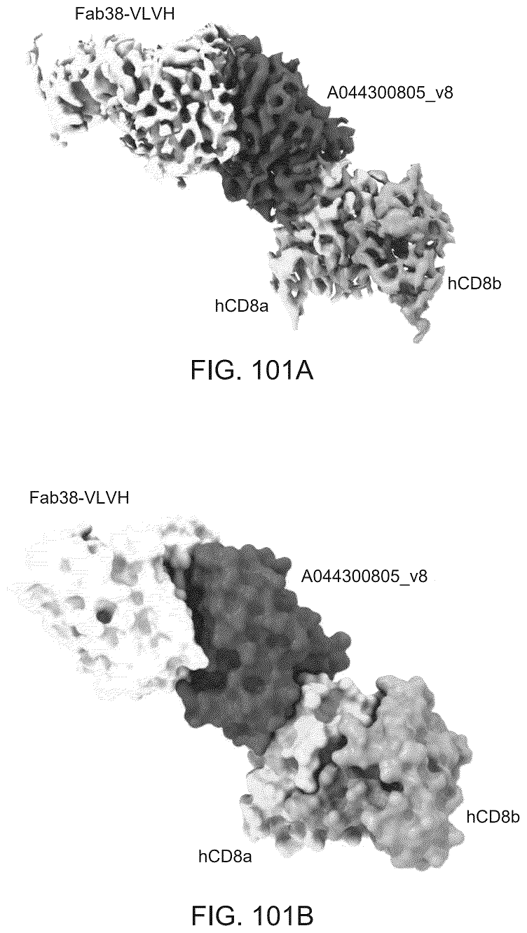

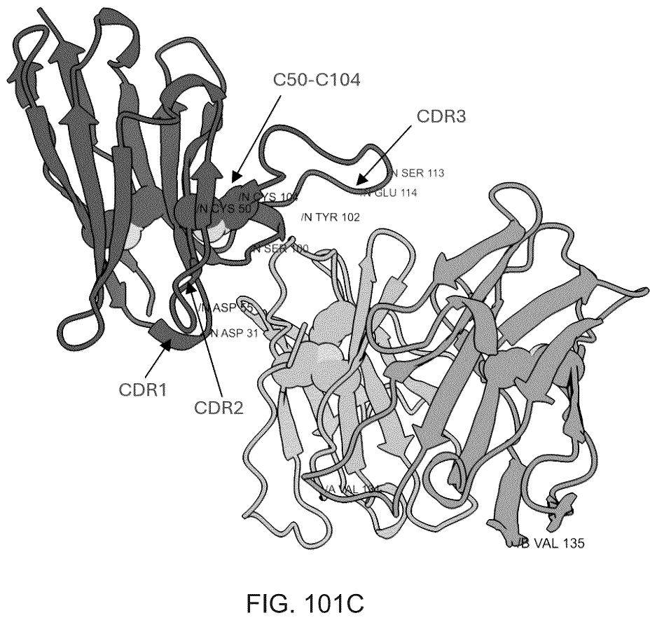

A view of the structure of the interaction of ISVD A044300805_v8 with CD8αβ is given in FIG. 101. ISVD A044300805_v8 binds to a distinct epitope at the apical region of the human CD8α chain. ISVD A044300805_v8 binding does not affect hCD8αβ dimeric structure.

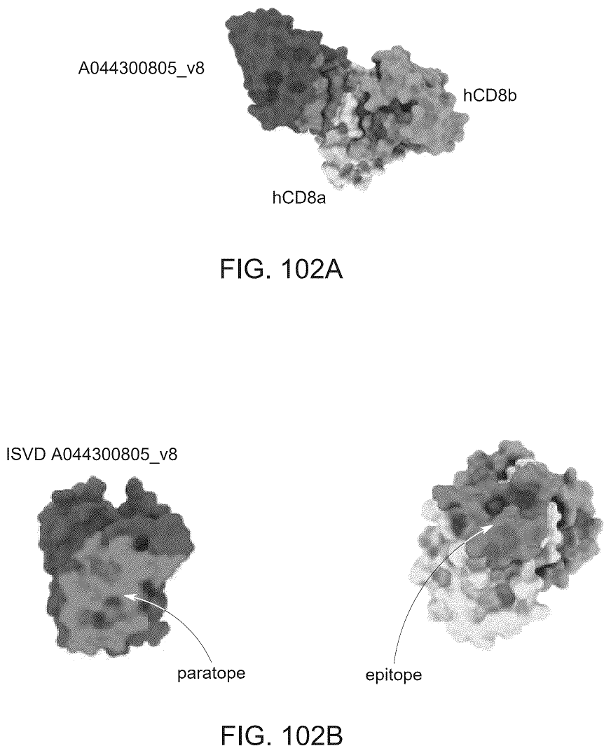

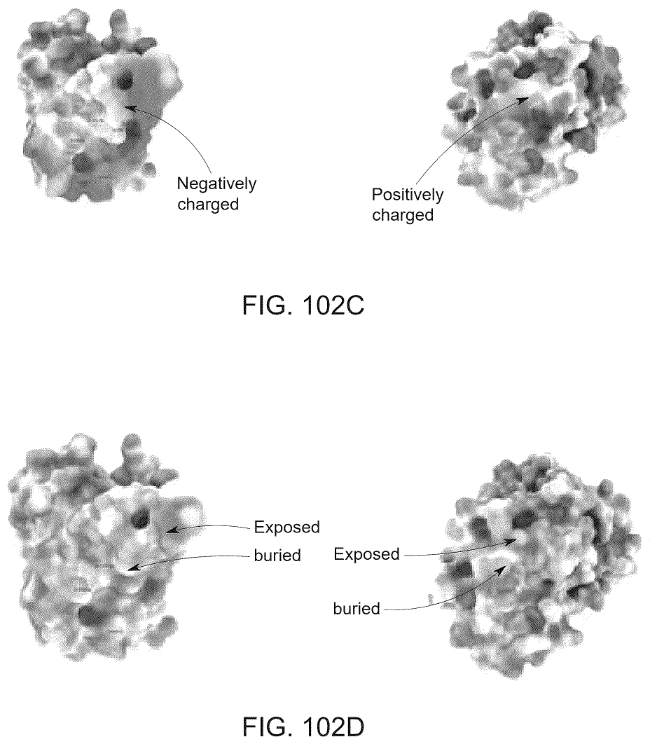

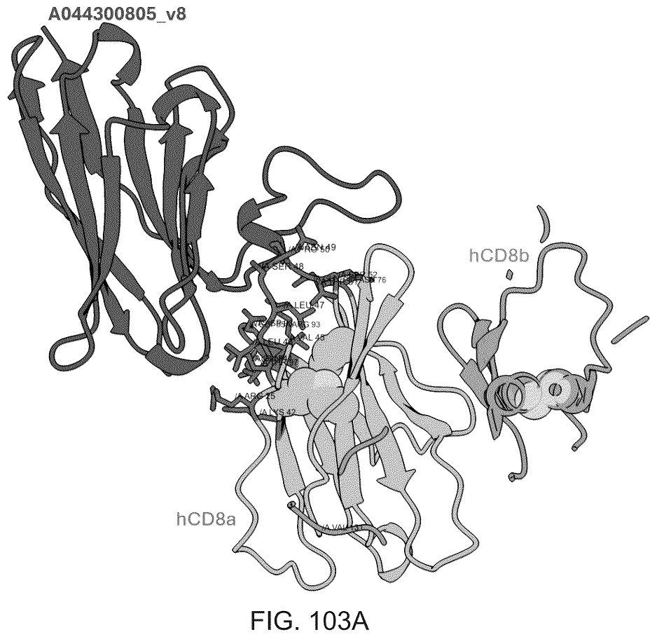

The binding interface (FIG. 102) spans 735.9 Å² (701.3 Å² on ISVD A044300805 v8 and 770.5 Å² on CD8α), which is substantial for a VHH-protein interaction. The binding features 11 hydrogen bonds and 5 salt bridges, with a ΔG of −1.7 kcal/mol. Large negatively charged patches make up the rim of the paratope region, which is highly complementary to hCD8α (FIG. 102B).

### CDR Engagement

All three complementarity-determining regions (CDRs) participate in target recognition:
CDR1 and CDR2 form critical contacts with CD8α through residues E30, D31, Y32 (E30, D31, Y32 according to Kabat numbering) (CDR1) and R⁵², Y54 (R⁵², Y53 according to Kabat numbering) (CDR2);
The extended CDR3 loop interacts extensively with the upper surface of CD8 via residues G99, S100, Y101, Y102, A103, C₁₀₄, A105, and Y106 (G95, S96, Y97, Y98, A99, C100, A100a, and Y100b according to Kabat numbering).

These residues are making critical contacts in the paratope-epitope interface. Typically, they have a high buried surface area (BSA>20-30 Å²), are involved in multiple interaction types (e.g., hydrogen bonds, hydrophobic contacts), and/or locate within the interface core.

CDR3 is stabilized by a non-canonical disulfide bond between C50 in CDR2 and C104 in CDR3 (C50 in CDR2 and C100 in CDR3 according to Kabat numbering) stabilizing the paratope conformation and creating a rigid binding scaffold that enhances specificity. This covalent linkage constrains CDR3 in a conformation optimal for CD8 recognition. The extended CDR3 amino acids 105-120 (100a-101 according to Kabat numbering) are flexible (FIG. 101C).

### Molecular Interactions

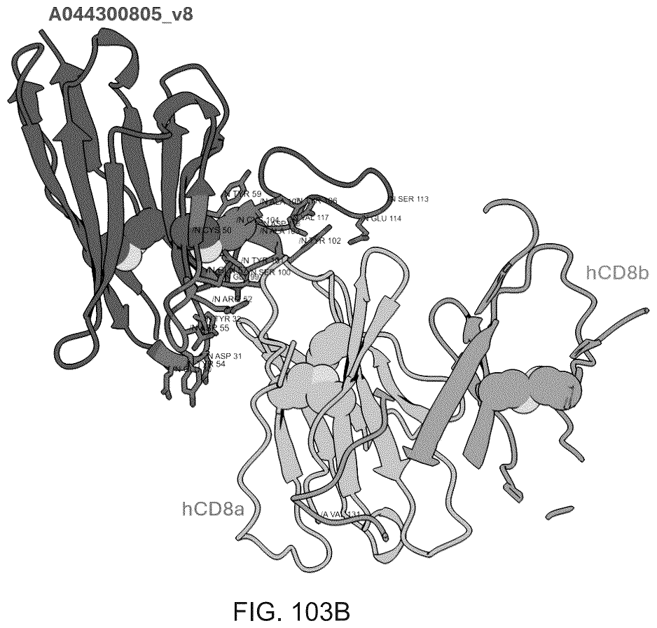

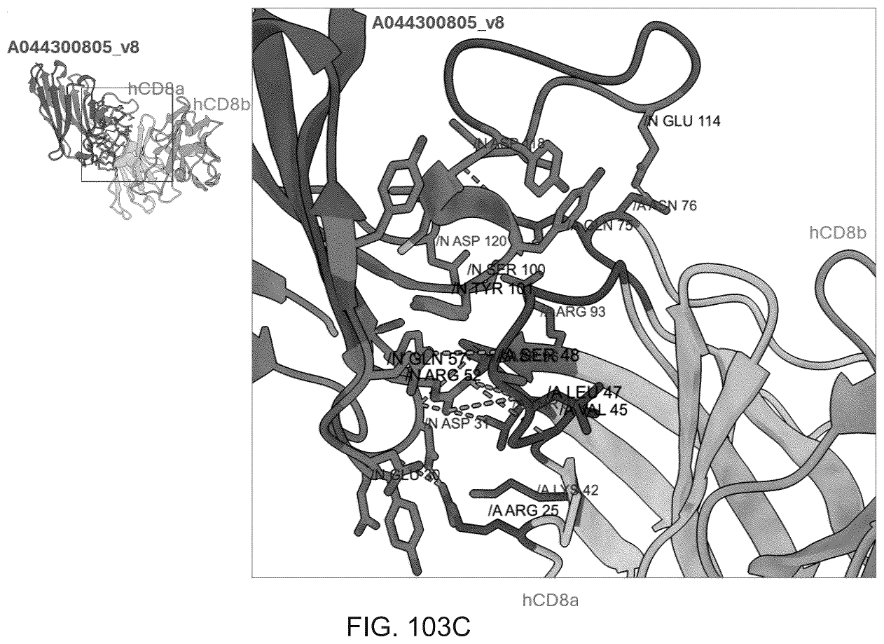

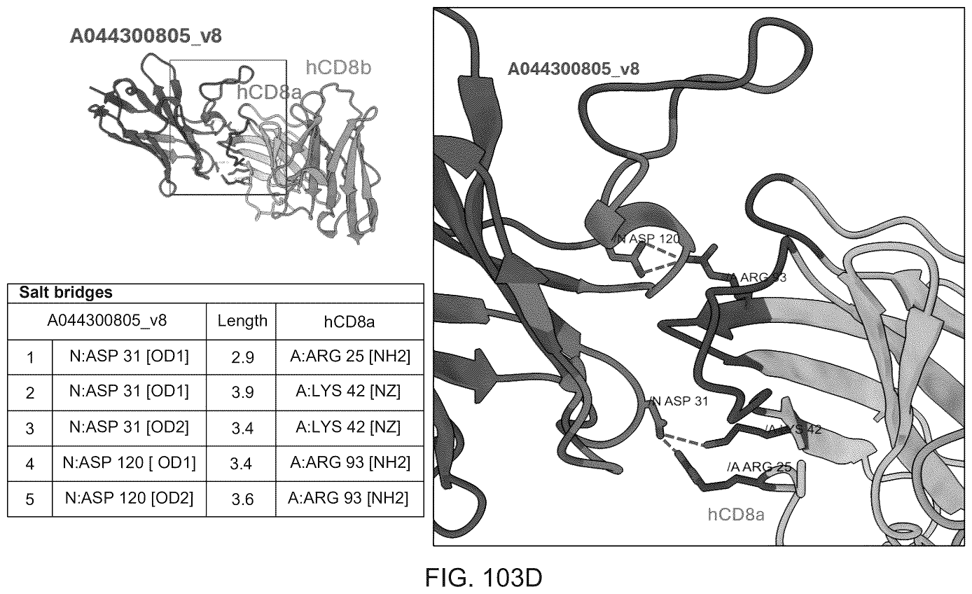

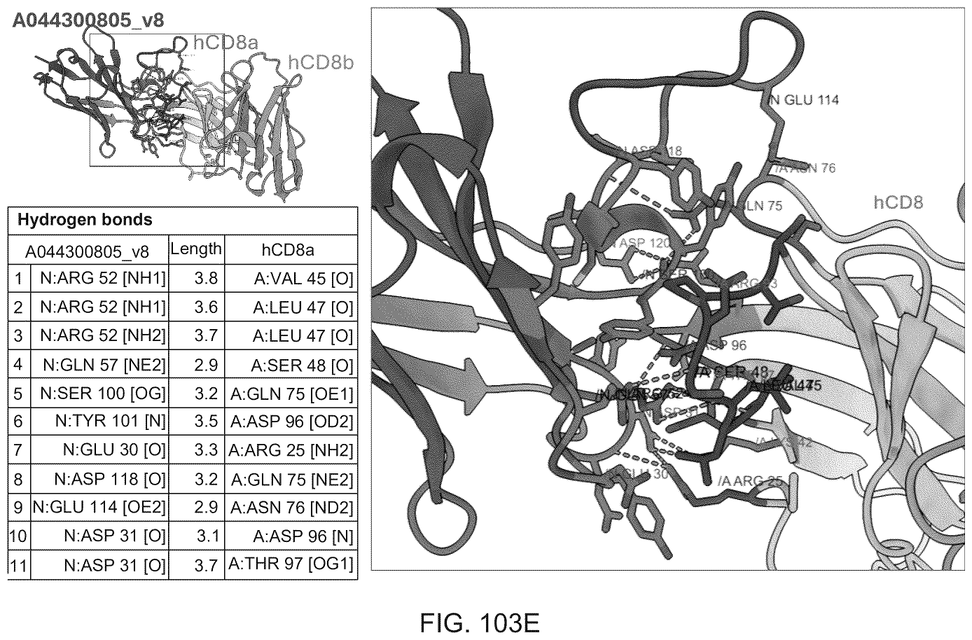

Analysis of the epitope-paratope interface (interaction site) showed involvement in binding (interacting residues with a distance of <4.0 Å) by 23 residues from ISVD A044300805 v8 (17.6% of total residues) and 19 residues from CD8 alpha (17.1% of total residues):
CD8-alpha epitope residues: R²⁵, K42, Q44, V45, L46, L47, S48, N49, P50, T51, S52, S74, Q75, N76, R⁹³, L94, G95, D96, T97 (FIG. 103A);
ISVD A044300805_v8 paratope residues: E30, D31, Y32, A33, R⁵², Y54, D55, Q57, Y59, G99, S100, Y101, Y102, A103, C₁₀₄, A105, Y106, E114, G115, V117, D118, L119, D120, which corresponds to E30, D31, Y32, A33, R⁵², Y53, D54, Q56, Y58, G95, S96, Y97, Y98, A99, C100, A100a, Y100b, E100j, G100k, V100m, D100n, L100o, D101, according to Kabat numbering (FIG. 103B).

A further detail of the amino acid interactions between the epitope on CD8α and the paratope in ISVD A044300805_v8 is given in FIG. 103C.

Five salt bridges were observed between acidic residues D31/D120 of ISVD A044300805_v8 D31/D101 according to Kabat numbering) and basic residues R²⁵/K42/R⁹³ of CD8α (FIG. 103D):

| ISVD A044300805_v8 | | Length | Amino acid in hCD8-alpha |
|---|---|---|---|
| Aa residue | Kabat numbering | (Å) | (SEQ ID NO:) |
| D31[OD1] | D31[OD1] | 2.9 Å | R25[NH2] |
| D31[OD1] | D31[OD1] | 3.9 Å | K42[NZ] |
| D31[OD2] | D31[OD2] | 3.4 Å | K42[NZ] |
| D120[OD1] | D101[OD1] | 3.4 Å | R93[NH2] |
| D120[OD2] | D101[OD2] | 3.6 Å | R93[NH2] |

[OD1]: one of the two carboxylate oxygen atoms in the acidic side chain of aspartate (D);
[OD2]: the second carboxylate oxygen atom in the side chain of aspartate (D);
[NH2]: the other terminal nitrogen in the guanidinium group of arginine (R);
[NZ]: terminal amino group nitrogen in the basic side chain of lysine (K).

11 hydrogen bonds were observed distributed across the interface, with key contributions from R⁵², Q57, S100, Y101, E114, and D118 of ISVD A044300805_v8 (FIG. 103E):

| ISVD A044300805_v8 | | Length | Amino acid in hCD8 |
|---|---|---|---|
| Aa residue | Kabat numbering | (Å) | (SEQ ID NO:) |
| R52[NH1] | R52[NH1] | 3.8 Å | V45[O] |
| R52[NH1] | R52[NH1] | 3.6 Å | L47[O] |
| R52[NH2] | R52[NH2] | 3.7 Å | L47[O] |
| Q57[NE2] | Q56[NE2] | 2.9 Å | S48[O] |
| S100[OG] | S96[OG] | 3.2 Å | Q75[OE1] |
| Y101[N] | Y97[N] | 3.5 Å | D96[OD2] |
| E30[O] | E30[O] | 3.3 Å | R25[NH2] |
| D118[O] | D100n[O] | 3.2 Å | Q75[NE2] |
| E114[OE2] | E100j[OE2] | 2.9 Å | N76[ND2] |
| D31[O] | D31[O] | 3.1 Å | D96[N] |
| D31[O] | D31[O] | 3.7 Å | T97[OG1] |

[OD2]: the second carboxylate oxygen atom in the side chain of aspartate (D);
[OE1]: one of the two carboxylate oxygen atoms in the side chain of glutamate (E);
[OE2]: the second carboxylate oxygen atom in the side chain of glutamate (E);
[NH1]: one of the terminal nitrogen atoms in the guanidinium group of arginine (R);
[NH2]: the other terminal nitrogen in the guanidinium group of arginine (R);
[NE2]: terminal nitrogen in amide of glutamine (Q) or imidazole ring of histidine (H);
[ND2]: terminal nitrogen in the side-chain amide group of asparagine (N);
[OG]: hydroxyl oxygen atom in the polar side chain of serine (S);
[OG1]: hydroxyl oxygen atom in the threonine side chain of threonine (T);
[O]: backbone carbonyl oxygen (C═O);
[N]: backbone amide nitrogen (—NH).

Hydrophobic interactions centered around Y102 (Y98 according to Kabat numbering) of ISVD A044300805_v8 and P50/L46 of CD8 alpha.

Key interactions include strong electrostatic contacts between CD8α residues R²⁵, K42, and R⁹³ with A044300805_v8 residues D31 and D120 (D31 and D101 according to Kabat numbering), forming salt bridges with distances of 2.9-3.9 Å. The binding is further stabilized by hydrogen bonds between R⁵² (A044300805_v)-L47 (CD8), Q57 (A044300805_v)-S48 (CD8), Y101 (A044300805_v)-D96 (CD8), S100 (A044300805_v)-Q75 (CD8), and E114 (A044300805_v)-N76 (CD8) corresponding to R⁵² (A044300805_v)-L47 (CD8), Q56 (A044300805_v)-S48 (CD8), Y97 (A044300805_v)-D96 (CD8), S96 (A044300805_v)-Q75 (CD8), and E100j (A044300805_v)-N76 (CD8) according to Kabat numbering.

Residue A: PRO50 exhibited the most stabilizing effect on the protein-protein interface, with a solvent-accessible surface area (ASA) of 118.00 Å², a buried surface area (BSA) of 93.23 Å², and a solvation free energy contribution (ΔG) of +1.34 kcal/mol. Residue A: LEU46 also contributed to interface stabilization, with an ASA of 88.83 Å², BSA of 65.45 Å², and ΔG of +1.05 kcal/mol. In contrast, residue A: GLN44 showed the most destabilizing effect, with an ASA of 36.76 Å², BSA of 33.77 Å², and a ΔG of −0.43 kcal/mol.

### Biomimetic Recognition

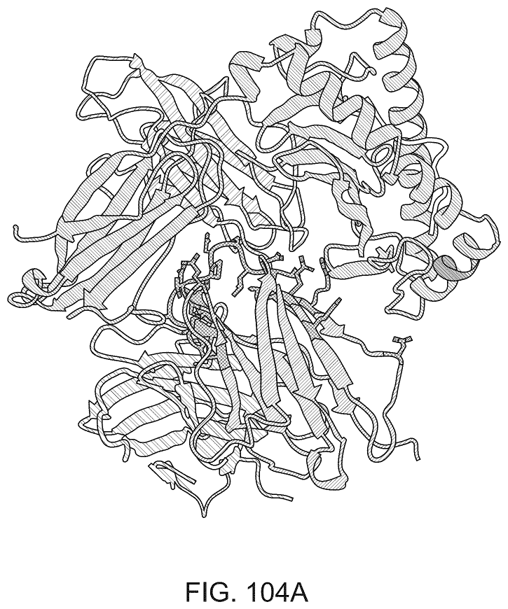

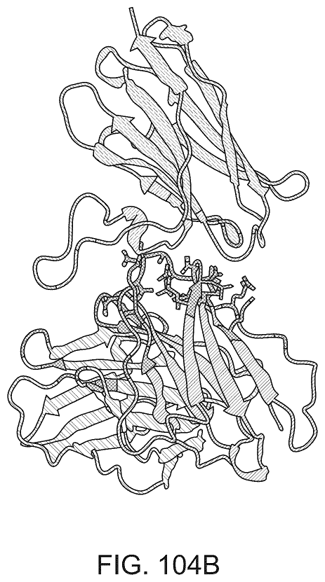

ISVD A044300805_v8's binding mode utilizing the long CDR3 shows a form of structural mimicry of the β2-microglobulin/MHC class I interaction with CD8 (FIG. 104A). The epitope on CD8 alpha overlaps partially with MHC class I binding residues. Furthermore, the curved architecture formed by the extended CDR3 recapitulates the contour of MHC molecules that naturally engage CD8 on the top, suggesting a maximization of optimal recognition surface by ISVD A044300805_v8 (FIG. 104B).

### Example 105: HDX-MS (Hydrogen-Deuterium Exchange Mass Spectrometry) of Human CD8 Alpha/Beta Dimer in Complex with ISVD A044300805_v8

### Methods

HDX-MS experiments were performed using a nanoACQUITY UPLC M-Class system (Waters Corporation, Milford, MA) equipped with a HDX manager coupled to an Orbitrap Fusion Tribrid mass spectrometer (Thermo Fisher Scientific, San Jose, CA).

The CD8 α/β-ISVD A044300805_v8 complex was formed by incubating 15 μl of 30 μM CD8 α/β with 5 μl of ISVD A044300805_v8 at 108 μM in PBS, pH 7.4. Control samples were prepared by replacing the VHH and CD8 solution with PBS for epitope and paratope determination, respectively.

Deuterium labelling was initiated by diluting the protein solutions with 110 μl of deuterated PBS, pD 7.4 resulting in 85% D20. Deuterated samples were incubated for 10 sec, 1 min, 10 min and 60 min. The exchange reaction was quenched by adding 40 μl of an ice-cold quench buffer (0.5% HCOOH, 4 M Urea, 150 mM TCEP, pH 2.5) to 20 μl of the deuterated solution. Quenched samples were immediately snap frozen in liquid nitrogen and stored at −80° C. until LC/MS analysis. A undeuterated control was obtained by replacing the deuterated PBS solution with a PBS solution.

Quenched samples were quickly thawed, incubated for 2 minutes on ice and immediately injected into a nepenthesin-II/pepsin mixed column (Affipro, Prague, Czech Republic) held at 20° C. for online digestion. The resulting peptides were desalted on a C18 trap column and separated on an analytical C18 column (ACQUITY UPLC BEH C18, 1.7 μm, 1.0×100 mm, Waters) using a 7-minute gradient of 5-40% acetonitrile in 0.1% formic acid at a flow rate of 35 μL/min. Mass spectra were acquired in positive ion mode over the range m/z 325-1200 with a resolution of 60,000 at m/z 200. Peptide identification was performed using non-deuterated samples with data-dependent MS/MS using higher-energy collisional dissociation (HCD). MS/MS spectra were searched against a database containing the sequences of CD8α, CD8β, and ISVD A044300805_v8 using Thermo Scientific™ BioPharma Finder™ 2.0 software (Thermo Scientific™).

Deuterium incorporation was determined using HDExaminer software ((Version 3.4.0, Trajan scientific and medical). The difference in deuterium uptake between CD8 α/β alone and in complex with ISVD A044300805_v8 and between ISVD A044300805_v8 alone and in complex with CD8 was calculated for each peptide at each time point. Peptides with an average of all differences integrated over all deuteration time points greater than 10% and expanding at least 3 consecutive regions were considered significant.

The HDX-MS results were mapped onto the cryoEM structure of the CD8 α/β-ISVD A044300805_v8) complex (3.3 Å resolution) to visualize regions of protection upon ISVD A044300805_v8) binding, providing complementary information to the interface analysis from the structural data.

### Molecular Interactions

The paratope-epitope interface determined by HDX-MS was consistent with the CryEM data. The interface involves 22 paratope residues from ISVD A044300805 v8 and 24 epitope residues from CD8 alpha:

ISVD A044300805_v8 paratope region determined by HDX-MS:
CDR1 region (27-32): F27, T28, F29, E30, D31, Y32 which corresponds to F27, T28, F29, E30, D31, Y32, according to Kabat numbering;
CDR2 region (51-57): I51, R⁵², T53, Y54, D55, E56, Q57, T58 which corresponds to 151, R⁵², T52a, Y53, D54, E55, Q56, T57, according to Kabat numbering;
CDR3 region (94-100a): A98, G99, S100, Y101, Y102, A103, C104, A105, which corresponds to A94, G95, S96, Y97, Y98, A99, C100, A100a, according to Kabat numbering.

CD8 epitope region determined by HDX-MS:
Region 44-46: Q44, V45, L46.
Region 47-54: L47, S48, N49, P50, T51, S52, G53, C₅₄.
Region 71-77: L71, Y72, L73, S74, Q75, N76, K77.
Region 93-98: R⁹³, L94, G95, D96, T97, F98

### Example 106: Preparation of a DSPE-PEG3.4K-15C01 Conjugate

106.1. Expression of 15C01 with GCC tag

For the expression of GCC tagged V_{HH}15C01 in P. pastoris, the yeast expression vectors contain the AOX1 promoter and terminator, a resistance gene for Zeocin and the coding information for the Saccharomyces cerevisiae α-mating factor signal peptide was used. P. pastoris cells (NRRL Y-11430 cells; ATCC 76273) containing the 15C01-GGC (amino acid sequence of SEQ ID NO. 490) encoding vector construct were grown in glycerol fed batches and induction was initiated by the addition of methanol. Standard fed batch fermentations conditions were used.

### 106.2. Capture Step

The anti-CD8 VHH was first isolated from the harvest stream using the standard affinity chromatography on protein A resin. The product eluting from this affinity chromatography step was a mixture of anti-CD8 VHH monomers and dimers. The amount of dimers was quantified by size exclusion HPLC (SE-HPLC). 58% of VHH was a dimer.

### 106.3. Addition of One Reduction Step Before the Polish Step Increases Conjugation Yield

In this example, the influence of a reduction step before the polish step on the conjugation yield was tested. Two experiments were conducted, one with and one without a reduction step before the polish step.

104.3.1 Experiment without Reduction Step Before the Polish Step

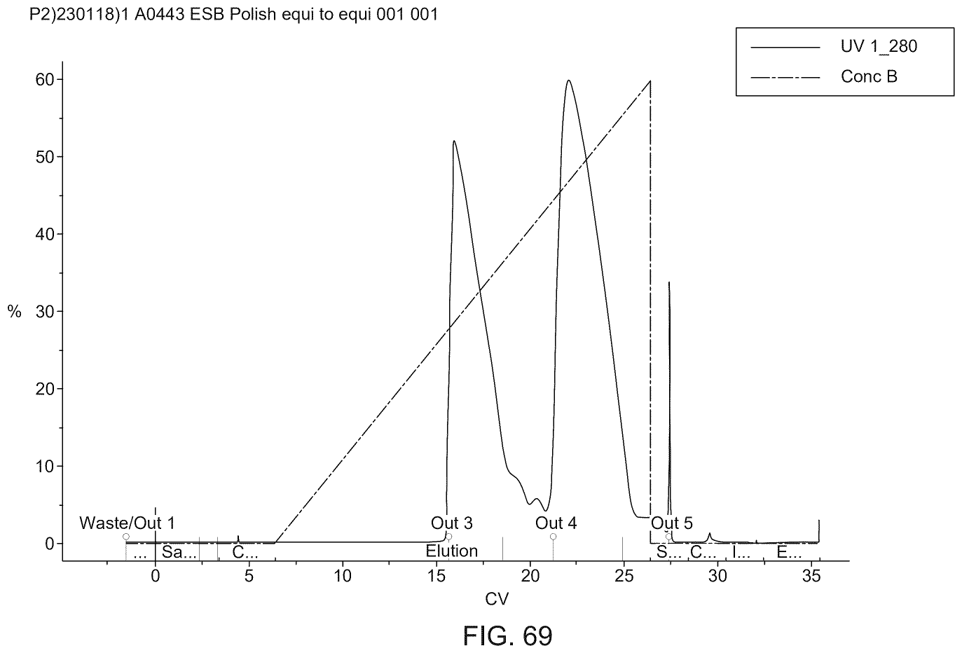

In a first experiment, the product obtained in the capture step was immediately purified by anion exchange chromatography (AEX) on Capto Q ImpRes (from Cytiva). After equilibration in 25 mM Tris pH7.5, the product (i.e. mixture of monomer and dimer) adjusted at pH 7.5±0.2 was loaded on the column. After washing the column with the same 25 mM Tris pH7.5 buffer, the product was eluted with a salt gradient from 0 to 2000 mM of NaCl. FIG. 69 shows the chromatographic profile obtained. The AEX chromatographic profile showed two peaks corresponding to the monomeric and the dimeric fractions. The monomers elute first from the column, followed by the dimers.

In this first experiment the dimeric fraction, i.e., the second peak eluting from the AEX chromatography, was further processed. The dimeric product was reduced using a final concentration of TCEP of 5 mM, for 90 min at room temperature under stirring. After reduction, TCEP was removed by UF/DF with a cut off of 10 kDa with seven diafiltration buffer volumes of PBS pH 7.4 with 5 mM EDTA. Analysis of the product by SE-HPLC showed complete reduction of the product, where monomers represented more than 98%.

Conjugation was done by addition of the lipid mixture containing DSPE-PEG2k and DSPE-PEG3.4K-Malemide. The molar ratio of ‘DSPE-PEG2k:DSPE-PEG3.4K-Malemide’ was ‘1:4’. Product concentration was first adjusted to 2 mg/mL with PBS pH 7.4, 5 mM EDTA and heated at the temperature of 37° C. The required volume of lipid mix was then added in order to reach the molar ratio ‘VHH:DSPE-PEG2K:DSPE-PEG3.4K-Malemide’ of ‘1:1:4’. After an incubation of 2 h under stirring, the conjugation was stopped by addition of 15 mM of free Cysteine with 5 mM of EDTA for 15 min at 37° C.

Finally, a second UF/DF with a cut off of 100 kDa was done on the conjugated product. The goal of this final step was to remove unconjugated free anti-CD8 VHH that flows through the UF/DF membrane, when the conjugated VHH in micellar form is retained. During this UF/DF 100 kDa, the buffer was exchanged to 25 mM HEPES pH 7.4, 150 mM NaCl. The conjugation yield determined by UV was 66%.

104.3.2 Experiment with a Reduction Step Before the Polish Step

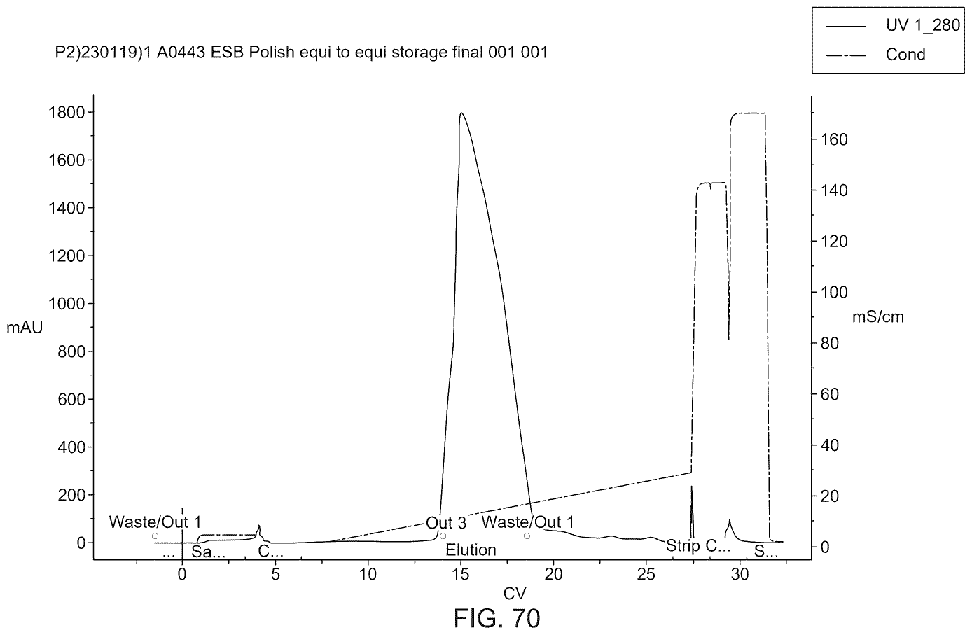

In a second experiment, the affinity protein A chromatography eluate was submitted to a first reduction step with 5 mM of TCEP for 1.5 h at room temperature under stirring. After reduction, the product was immediately purified by AEX using the same protocol as described for the first experiment (see 104.3.1). The chromatogram (FIG. 70) showed a single peak corresponding to the monomeric fraction obtained after reduction. The eluate from the AEX contained 99% of monomer.

Following AEX, the product was reduced with 0.1 mM TCEP for 90 min at room temperature and under stirring. TCEP was then removed from the product with UF/DF 10 kDa and conjugated using the same protocol as described for the first experiment (see 104.3.1). The unconjugated product was removed by UF/DF 100 kDa as explained for the first experiment (see 104.3.1). The UV measurement showed an improved conjugation yield of 74%.

106. 4 Optimization of the Reduction Step after the Polish Step

In this example, the second reduction step, after polish and prior to conjugation, was optimized to increase the conjugation yield.

Prior to the conjugation step, the C-terminal cysteine of the GGC tag needs to be free for conjugation to the maleimide functional group of the lipid nanoparticle (LNP). Hence a reduction is needed after the polish chromatography to convert formed VHH dimers to monomers. As the VHH scaffold also contains internal disfulphide bonds, a balance must be struck between sufficient reduction and avoiding over-reduction of these canonical disulphide bonds in the VHH scaffold which could lead to potential overconjugation.

This reduction step after the polish step was optimized using the Design of Experiments (DoE) concept. The experimental design varied 4 factors:
1. pH and buffer system:
   a. 25 mM L-Histidine pH 6.0
   b. 25 mM BIS-TRIS pH 6.5
   c. 25 mM TRIS pH 7.5
2. TCEP concentration:
   a. 1× molar excess to Nb (i.e. equimolar quantity)
   b. 10× molar excess to Nb
   c. 20× molar excess to Nb
3. Conjugation incubation time:
   a. 1 h
   b. 2 h
   c. 16 h
   d. 20 h
4. EDTA concentration:
   a. No EDTA
   b. 5 mM EDTA

The full experimental design is displayed in Table 59.

TABLE 59
Experimental design of the optimization of the reduction step
after the polish step. Four factors (conjugation incubation
time, buffer system and pH, EDTA concentration, and TCEP
concentration) were tested in a DoE experimental set up.

| | Incubation | Buffer | EDTA | TCEP [molar |
|---|---|---|---|---|
| Experiment | time [hours] | pH | [mM] | excess to Nb] |
| 1 | 2 | 7.5 | 0 | 20 |
| 2 | 1 | 7.5 | 5 | 1 |
| 3 | 20 | 7.5 | 0 | 1 |
| 4 | 2 | 6 | 0 | 20 |
| 5 | 1 | 6.5 | 0 | 20 |
| 6 | 20 | 6.5 | 0 | 10 |
| 7 | 20 | 6 | 0 | 1 |
| 8 | 1 | 6 | 0 | 1 |
| 9 | 2 | 6.5 | 0 | 10 |
| 10 | 16 | 7.5 | 5 | 1 |
| 11 | 2 | 6.5 | 0 | 1 |
| 12 | 16 | 6 | 0 | 1 |
| 13 | 1 | 7.5 | 0 | 10 |
| 14 | 1 | 6.5 | 5 | 1 |
| 15 | 20 | 7.5 | 0 | 20 |
| 16 | 16 | 6 | 5 | 20 |
| 17 | 2 | 7.5 | 0 | 1 |
| 18 | 2 | 6 | 5 | 1 |
| 19 | 20 | 6.5 | 5 | 20 |
| 20 | 20 | 6 | 5 | 1 |
| 21 | 20 | 6 | 0 | 20 |
| 22 | 2 | 6.5 | 5 | 20 |
| 23 | 1 | 6 | 5 | 20 |
| 24 | 16 | 7.5 | 5 | 20 |
| 25 | 16 | 6.5 | 0 | 20 |
| 26 | 16 | 7.5 | 0 | 10 |
| 27 | 20 | 7.5 | 5 | 10 |
| 28 | 16 | 6.5 | 5 | 10 |
| 29 | 1 | 7.5 | 5 | 20 |
| 30 | 2 | 7.5 | 5 | 10 |

To evaluate the experiments, three assays were applied to all samples. The first analytical technique was SE-HPLC under non-reducing conditions to evaluate the reduction efficiency by determining the ratio of monomer and dimer VHH. The second analytical technique was RP—(U) HPLC under non-reducing conditions which can quantify the amount of missing canonical disulphide bridges due to overreduction. The last assay was to verify whether there was limited residual TCEP present after stopping the reduction reaction, as this could alter the monomer-dimer ratio before analysis.

The protocol was started with dimerized VHH at a concentration of 2.2 mg/mL. The material was buffer exchanged to their respective pH and buffer system each containing 230 mM NaCl. EDTA was added from a 500 mM stock solution to some samples according to the experimental design. Reduction reaction was initiated by adding the appropriate amount of TCEP from a 400 mM stock solution. Incubation of the 1.6 mL total reaction volume was done at room temperature under mixing conditions in a Thermoshaker at 300 RPM. The reduction was stopped by removing the TCEP from the VHH using a 4 kDa MWCO 10 mL Zeba Spin Desalting Column to buffer exchanging it to D-PBS containing 5 mM EDTA. Samples for residual TCEP analysis were taken after this buffer exchange. The bulk of the reduced material was subsequently capped by adding 2-Iodoacetamide (IAA) in highly purified water from a 375 mM freshly made stock solution to a final concentration of 17.85 mM. The capping fixates the monomer-dimer ratio and prevents further overconjugation before analysis on SE-HPLC and RP—(U) HPLC. Results are demonstrated in Table 60.

TABLE 60
Results of the DoE experiment to optimize reduction process after polish step.

| | | | TCEP | | | |
|---|---|---|---|---|---|---|
| | Time | EDTA | (molar | SE-HPLC | RP-(U)HPLC | Residual TCEP |
| Buffer pH | (h) | (mM) | excess) | (% monomer) | (% missing S-S) | (mM) |
| His pH 6.0 | 1 | 0 | 1 | 6.61 | 1.44 | <QL |
| His pH 6.0 | 2 | 5 | 1 | 15.75 | 2.02 | <QL |
| His pH 6.0 | 16 | 0 | 1 | 70.43 | 3.58 | <QL |
| His pH 6.0 | 20 | 0 | 1 | 42.07 | 3.46 | <QL |
| His pH 6.0 | 20 | 5 | 1 | 47.12 | 3.28 | <QL |
| His pH 6.0 | 1 | 5 | 20 | 29.25 | 2.79 | 0.009 |
| His pH 6.0 | 2 | 0 | 20 | 57.00 | 3.30 | <QL |
| His pH 6.0 | 16 | 5 | 20 | 89.15 | 3.76 | 0.066 |
| His pH 6.0 | 20 | 0 | 20 | 94.93 | 3.59 | 0.073 |
| BIS-TRIS pH 6.5 | 1 | 5 | 1 | 22.24 | 2.51 | <QL |
| BIS-TRIS pH 6.5 | 2 | 0 | 1 | 20.63 | 2.11 | <QL |
| BIS-TRIS pH 6.5 | 2 | 0 | 10 | 26.77 | 2.64 | <QL |
| BIS-TRIS pH 6.5 | 16 | 5 | 10 | 98.35 | 3.90 | 0.039 |
| BIS-TRIS pH 6.5 | 20 | 0 | 10 | 88.05 | 3.78 | 0.013 |
| BIS-TRIS pH 6.5 | 1 | 0 | 20 | 31.23 | 2.97 | <QL |
| BIS-TRIS pH 6.5 | 2 | 5 | 20 | 73.30 | 3.86 | 0.030 |
| BIS-TRIS pH 6.5 | 16 | 0 | 20 | 98.21 | 3.91 | 0.063 |
| BIS-TRIS pH 6.5 | 20 | 5 | 20 | 98.58 | 4.18 | 0.055 |
| TRIS pH 7.5 | 1 | 5 | 1 | 31.72 | 2.85 | <QL |
| TRIS pH 7.5 | 2 | 0 | 1 | 49.87 | 3.68 | <QL |
| TRIS pH 7.5 | 16 | 5 | 1 | 90.91 | 3.31 | <QL |
| TRIS pH 7.5 | 20 | 0 | 1 | 84.60 | 3.51 | <QL |
| TRIS pH 7.5 | 1 | 0 | 10 | 45.22 | 2.07 | <QL |
| TRIS pH 7.5 | 2 | 5 | 10 | 85.36 | 3.67 | 0.023 |
| TRIS pH 7.5 | 16 | 0 | 10 | 98.84 | 4.10 | 0.021 |
| TRIS pH 7.5 | 20 | 5 | 10 | 99.03 | 3.94 | 0.011 |
| TRIS pH 7.5 | 1 | 5 | 20 | 52.78 | 4.45 | 0.017 |
| TRIS pH 7.5 | 2 | 0 | 20 | 84.07 | 3.99 | 0.105 |
| TRIS pH 7.5 | 16 | 5 | 20 | 99.01 | 4.68 | 0.050 |
| TRIS pH 7.5 | 20 | 0 | 20 | 98.83 | 4.42 | 0.024 |

The relative peak area related to overconjugation on RP—(U) HPLC analysis showed little variation as all experiments resulted in an acceptable overconjugation between 1.44% and 4.68%. There were no restrictions on the process parameters in the design space based on the overconjugation risk.

The removal of TCEP to stop the reduction reaction was confirmed by the low TCEP quantification with the highest concentration being 0.105 mM.

The buffer system and pH played a role in an effective reduction. The higher the pH, the faster and thus the better was the reduction. The same applies for the TCEP concentration, higher amounts of TCEP corresponded to a faster and more robust reduction. For incubation times of 1 h and 2 h timepoint, there was a high variability ranging from 31.72% monomer to 85.36% at pH 7.5. While the reduction was much more robust at pH 7.5 for timepoints between 16 and 20 hours, reflected by a narrower range of monomer ranging from 84.60% to 99.03%.

The optimal process parameters for the second reduction (after the polish step) were defined as 25 mM TRIS pH 7.5, combined with a reduction time between 16-20 hours and 20 times molar excess of TCEP for robustness. 2.5 mM EDTA was still added as a pre-caution to prevent oxidation potentially leading to dimerization.

### 106.5 Removal of TCEP

### 106.5.1 Removal of TCEP is Necessary Before Conjugation

In this example, the influence of TCEP on the conjugation of the anti-CD8 VHH with the DSPE-PEG-3.4k-Malemide was investigated. Purified anti-CD8 VHH was reduced with increasing concentrations of TCEP for 1.5 hour at room temperature. Following the reduction and buffer exchange, the anti-CD8 VHH was conjugated with a lipid mix of DSPE-PEG-2k and DSPE-PEG-3.4k-Malemeide in order to form the anti-CD8 VHH-DSPE-PEG-3.4k conjugate in micellar form. Conjugation was done at 37° C. for 2 h under agitation. Table 61 below summarizes the results obtained.

TABLE 61
Levels of monomeric/dimeric anti-CD8 VHH after TCEP reduction and its influence
on the conjugation of the anti-CD8 VHH with DSPE-PEG-3.4k-Malemeide.

| | | | Concentration | Level of | Level of |
|---|---|---|---|---|---|
| | Level of | Level of | of TCEP at | unconjugated | conjugated |
| TCEP | monomer after | dimer after | beginning of | ant- | anti- |
| concentration | reduction | reduction | conjugation | CD8 VHH | CD8 VHH |
| (mM) | % | % | mM | % | % |
| 0.01 | 73.2 | 26.8 | <0.01 | 38 | 62 |
| 1.4 | 99.0 | 1.0 | 0.87 | 77 | 23 |
| 2.8 | 99.5 | 0.5 | 1.09 | 80 | 20 |

Increasing the concentration of TCEP improves the reduction of dimers to monomers. At the lower concentration of 0.01 mM TCEP, 73% of the product was monomeric. At higher concentration of 1.4 and 2.8 mM, at least 99% of the product was monomeric.

Following the reduction step, part of the TCEP was removed by chromatography. The concentration of residual TCEP after chromatography and before start of conjugation was lower than 0.01 mM when 0.01M TCEP was used for the reduction step and 0.87 and 1.09 mM when higher concentrations of TCEP had been used for the conjugation step. This higher of amount of residual TCEP negatively impacted the conjugation reaction analysed by Reverse Phase-HPLC connected to a CAD detector. In absence of TCEP (<0.01 mM), 62% of the anti-CD8 VHH was conjugated, and 38% did not react with the lipids. From this 38% of unreacted product, 27% was a dimer that had no free Cysteine for conjugation. On the other hand, at higher TCEP concentration with more than 99% of monomer free for conjugation, only approximately 20% of the anti-CD8 VHH conjugated with the lipids while 80% of free product did not react with the DSPE-PEG-3.4k-Maleimide. These data demonstrate that complete removal of TCEP is necessary for the successful conjugation of the anti-CD8 VHH with the DSPE-PEG-3.4k Maleimide.

### 106.5.2 Buffering the VHH in PBS Avoids Dimerization of the VHH

Stability of the monomeric anti-CD8 VHH in the absence of TCEP was evaluated in this example. In a first experiment, removal of TCEP with UF/DF 10 kDa was demonstrated. The concentration of TCEP was measured in the permeate sample at different timepoints of the UF/DF (Table 62).

TABLE 62
Concentration of TCEP during the UF/DF 10 kDa.

| Sample | Level of TCEP (mM) |
|---|---|
| Before the start of Reduction | <0.01 |
| End of Reduction | 0.045 |
| Diafiltration Volume = 1 | 0.032 |
| Diafiltration Volume = 2 | 0.014 |
| Diafiltration Volume = 3 | <0.01 |
| Diafiltration Volume = 4 | <0.01 |
| Diafiltration Volume = 5 | <0.01 |
| Final UFDF pool after 5 Diafiltration volumes | <0.01 |

After 3 diafiltration volumes, a complete clearance of TCEP was observed.

In a second experiment, reduced anti-CD8 VHH was submitted to UF/DF 10 kDa. During this UF/DF, TCEP was completely removed and the anti-CD8 VHH was buffered in 2.68 mM KCl, 1.47 mM KH2PO4, 136.9 mM NaCl, 8.10 mM Na2HPO4 pH 7.4 with 2.5 mM of EDTA. This buffered product without any TCEP was put on hold for 14 days at different temperatures without agitation and the stability of the monomeric form (in the absence of TCEP) was measured at different time points (Table 63).

TABLE 63
Percentage of monomeric anti-CD8 VHH without
TCEP in phosphate buffer at different temperatures,
measured at different time intervals.

| | Time | Level of |
|---|---|---|
| Temperature [° C.] | [days] | monomer [%] |
| Start material for all three temperatures | 0 | 98.5 |
| Room temperature | 1 | 97.9 |
| Room temperature | 3 | 96.8 |
| Room temperature | 14 | 93.2 |
| 2-8 | 1 | 98.4 |
| 2-8 | 3 | 97.7 |
| 2-8 | 14 | 96.6 |
| ≤−20 | 3 | 97.4 |
| ≤−20 | 14 | 96.8 |

Results in Table 63 show that the level of monomers remained above 96% for 3 days at the three temperatures. For longer hold times, where samples are normally stored at low temperatures of 2-8° C. or frozen at −20° C., the monomeric form remained above 96% for 14 days. These results show the stability of the anti CD8-VHH in the absence of TCEP in the selected buffer.

### 106.6 Conjugation Step

In this example, the conjugation step was optimized to increase the conjugation yield and increase the number of VHH molecules on the PEG-Micelle. As described above, before the conjugation, the anti-CD8 VHH polish eluate material was submitted to a reduction step followed by an UF/DF step to remove TCEP. Then, the anti-CD8 VHH was prepared for the conjugation step. Conjugation was based on the reaction of a lipid mixture containing DSPE-PEG2k-Methoxy and DSPE-PEG3.4K-Maleimide. DSPE-PEG3.4K-Maleimide is the reagent containing maleimide which is the functional moiety that drives the conjugation reaction. As a result of the conjugation step, a lipid micelle is obtained with anti-CD8 VHH conjugated to it. The conjugation step can be evaluated by means of the conjugation recovery (i.e., how many molecules of VHH have efficiently reacted) and by the DSPE-PEG2k-Methoxy: VHH ratio. This ratio, evaluated after UF/DF II, describes the micelle composition. It indicates the amount of VHH molecules that decorates the PEG-lipid micelle. The smallest this ratio is, the more VHH molecules decorate the PEG-lipid micelle, hence presenting more antiCD8 recognition sites.

The conjugation step was optimized using the Design of Experiments (DoE) concept. Two main goals were defined: a) to increase conjugation recovery; and b) to decrease the DSPE-PEG2k-Methoxy: VHH ratio.

The experimental design varied 7 factors:
1. pH and buffer system:
   a. 10 mM Sodium phosphate pH 7.5
   b. 10 mM Sodium citrate pH 6.5
2. NaCl concentration:
   a. 0 mM
   b. 70 mM
   c. 180 mM
   d. 250 mM
3. Ratio VHH:DSPE-PEG3.4K-Maleimide
   a. 1
   b. 2
   c. 3
   d. 4
4. Ratio DSPE-PEG3.4K-Maleimide:DSPE-PEG2K-Methoxy
   a. 1.5
   b. 4
5. V_{HH} concentration
   a. 0.5 mg/mL
   b. 3 mg/mL
   c. 6 mg/mL
   d. 10 mg/mL
6. Incubation step a. 2 h
   b. O/N (16-20 h)
7. Temperature
   a. RT
   b. 37° C.

The full experimental design was performed in 30 runs (conditions). To evaluate the experiments, SE-HPLC and RPC chromatography followed by UV/CAD detector were applied to the different samples. SE-HPLC under non-reducing conditions evaluated the reduction efficiency to confirm that a suboptimal reduction step resulting in a high dimer content was not impacting the conjugation outcome. RPC—UV followed the protein part of the conjugation whereas CAD detector studied the lipidic part.

The protocol started with anti-CD8 VHH material that had been submitted to the second reduction step. To remove the reducing agent, the reduced material was buffer exchanged with Zeba Spin Desalting Column 7K MWCO to their respective pH and buffer system. Protein concentration, pH, buffer system and NaCl concentration were adapted before conjugation for each condition. 5 mM EDTA was also added to each condition. Samples for analysis by SE-HPLC and RPC—UV under non-reducing conditions were taken after the buffer exchange. Before analysing, these samples were capped by adding 2-Iodoacetamide (IAA) in highly purified water from a 375 mM freshly made stock solution to a final concentration of 17.85 mM. The capping fixates the monomer-dimer ratio and prevents further overconjugation before analysis on SE-HPLC and RP—(U) HPLC.

The conjugation reaction was performed with two different PEG-Lipids: DSPE-3.4 kDa PEG-Maleimide and DSPE-2 kDa PEG-Methoxy. Initially, each lipid was resuspended separately to a 3.4 mM concentration in 1 mM citric acid pH 5.7 to generate two separate stock solutions.

For each condition, and based on factor #4 (Ratio DSPE-PEG2K:DSPE-PEG3.4K-Maleimide) values, the needed volume from the two separate stock solutions were mixed together. The appropriate volume of PEG-Lipid mix, following the defined ratio VHH:DSPE-PEG3.4K-Maleimide in Factor #3, was added to 1.6 mL of the VHH sample. The VHHs presented different concentration values, based on Factor #5. This preparation was mixed at 400 rpm in a ThermoMixer equipment and incubated at 21° C. (2 h or O/N) or 37° C. (2 h or O/N). Overnight conditions never exceeded 20 h.

After the incubation time, the conjugation reaction was stopped by quenching it with L-Cysteine. The appropriate volume of a stock solution of 15 mM L-Cysteine+5 mM EDTA in D-PBS was added to achieve a final concentration of 1.5 mM L-Cysteine. Samples were mixed at 37° C. for 15 minutes while shaking at 400 rpm in Thermomixer during quenching step. After the incubation time, the samples for RPC—UV and RPC-CAD were taken.

The samples after the reduction step and subsequent buffer exchange presented a monomer percentage higher than 93% measured by non-reduced SE-HPLC. This confirms that the reduction step was sufficient to proceed with the conjugation. The reduction step also did not generate missing disulphide brides based on RP—(U) HPLC results.

RPC—UV values from quenched conjugated samples showed a relative peak area of conjugated anti-CD8 VHH with a variation between 21% and 70%. Free anti-CD8 VdHH (i.e. anti-CD8 VHH molecules that have not been conjugated) presented a peak area with a wide variation, with values from 26% to 85%. Peak area related to overconjugation presented limited variation with values ranging from 2% to 8%.

Information on the DSPE-PEG2k-Methoxy: VHH ratio in the samples was obtained by combining RPC—UV and RPC-CAD data. The highest value that was obtained in the DoE was 24 and the lowest one was 1.

Overconjugation evolution was also evaluated. Overconjugation is undesired and any conditions that would favour it should be discarded.

To increase the conjugation recovery, there were two key parameters: the VHH concentration and the ratio VHH:DSPE-PEG3.4K-Maleimide. The conjugation recovery response (i.e., percentage of conjugate VHH) followed an inverted-U-shaped curve for both parameters. Regarding the VHH concentration, the maximum conjugation recovery was achieved when the conjugation was performed between 3-4.5 g/L. Regarding the ratio VHH:DSPE-PEG3.4K-Maleimide, the maximum conjugation recovery was achieved with a ratio of 2.8. With higher ratios, from 3 onwards, not only the conjugation recovery decreased but also the overconjugation increased.

The ratio of the conjugation reagents impacted the micelle composition. The ratio DSPE-PEG2K-Methoxy: VHH ratio decreased almost in a linear way when the ratio VHH:DSPE-PEG3.4K-Maleimide also decreased or when using smaller DSPE-PEG3.4K-Maleimide: DSPE-PEG2K-Methoxy ratios. A variation in DSPE-PEG3.4k-Maleimide: DSPE-PEG2K-Methoxy ratio did not impact the overconjugation neither the conjugation recovery. However, a decrease in VHH: DSPE-PEG3.4K-Maleimide, from 2.3 downwards, presented a negative impact on conjugation recovery, going down until 50%.

The presence or absence of NaCl, buffer system, pH selected, incubation time and temperatures were factors that showed no impact on the conjugation recovery or micelle composition based on DoE results.

Based on the different results obtained, we defined the optimal process conditions for conjugation. The conjugation reagents are preferably added to a VHH solution in D-PBS+2.5 mM EDTA pH 7.4 with a protein concentration between 5.0-7.0 g/L (before conjugation), 3-4.5 g/L during conjugation. The ratio of the reagents is preferably 1 mol V_{HH}: 2 mol DSPE-PEG3.4K-Maleimide: 3 mol DSPE-PEG2K-Methoxy. The conjugation reaction is preferably stirred at 20-22° C. during 105-135 minutes. Afterwards, the conjugation reaction is preferably stopped by quenching it with L-Cysteine. The appropriate volume of a stock solution of 15 mM L-Cysteine+5 mM EDTA in D-PBS is preferably added to achieve a final concentration of 1.5 mM L-Cysteine. The sample is preferably mixed at 37° C. for 15 minutes while shaking at 400 rpm in Thermomixer.

### Example 107. In Vitro Transcription (IVT)

In vitro transcription reaction is using enzymatic reaction transcribing mRNA from DNA template.

Reaction mixture contains DNA template, ribo-nucleotides, enzyme, RNase inhibitor and corresponding buffer. DNA template must contain polymerase promoter. DNA template can be either linearized plasmid DNA, or PCR product, or annealed complimentary oligonucleotides.

### Typical IVT Protocol:

1. Linearize plasmid DNA template, ensuring that linearization site is at the desired 3′end of mRNA to be synthesized.
2. Purify linearized DNA.
3. Mix reaction mixture and incubate at 35-55C for 1-3 h
4. DNAse treatment of the reaction mixture to remove DNA template.
5. Purify mRNA from the reaction mixture.

Example using T7 polymerase.

This reaction produces 100-200 μg of mRNA using 1 μg DNA template.
1. Assemble the reaction at room temperature in the following order:

| REAGENT | AMOUNT | | FINAL CONCENTRATION |
|---|---|---|---|
| Nuclease-free water | X | μl | |
| 10X Reaction Buffer | 2 | μl | |
| ATP (100 mM) | 2 | μl | 10 mM final |
| GTP (100 mM) | 2 | μl | 10 mM final |
| UTP (100 mM) | 2 | μl | 10 mM final |
| CTP (100 mM) | 2 | μl | 10 mM final |
| Template DNA | X | μl | 1 μg |
| T7 RNA Polymerase Mix | 2 | μl | |
| Total Reaction Volume | 20 | μl | |

2. Mix thoroughly, pulse-spin in microfuge. Incubate at 37° C. for 2 hours.
3. DNase treatment to remove DNA template. Add 70 μl nuclease-free water, 10 μl of 10× DNase I Buffer, and 2 μl of DNase I (RNase-free), mix and incubate for 15 minutes at 37° C.
4. Proceed with purification of synthesized RNA or analysis of transcription products by gel electrophoresis.

Resulting mRNA will have 5′-triphosphate and can be enzymatically capped.

It is also possible to generate co-transcriptionally capped mRNA. In this case reaction should include cap analog (eg. CleanCap), and reaction mixture should be assembled as following:

| REAGENT | AMOUNT | | FINAL CONCENTRATION |
|---|---|---|---|
| Nuclease-free water | X | μl | |
| 10X Reaction Buffer | 2 | μl | |
| ATP (100 mM) | 2 | μl | 10 mM final |
| UTP (100 mM) | 2 | μl | 10 mM final |
| CTP (100 mM) | 2 | μl | 10 mM final |
| GTP (20 mM) | 2 | μl | 2 mM final |
| Cap Analog (40 mM) | 4 | μl | 8 mM final |
| Template DNA | X | μl | 1 μg |
| T7 RNA Polymerase Mix | 2 | μl | |
| Total Reaction Volume | 20 | μl | |

### Example 108. Reprogramming CAR-T Cells In Vivo Using aCD8-Targeted mRNA-LNP to Treat Hematological Malignancies

### Methods

### LNP Preparation

LNPs were formulated with an encapsulated mRNA payload and lipid blend by mixing an aqueous mRNA solution and an ethanolic lipid solution using an in-line microfluidic mixing process. The mRNA (encoding eGFP or CAR) was mixed with pH 4 acetate buffer to provide a final aqueous mRNA solution containing 300 μg/mL mRNA and 65 mM acetate buffer. The lipid components ALC-0315, Lipid 10, Lipid 15, Lipid 16, Lipid 24, or Lipid 26 (Organix Inc, Massachusetts, US), Cholesterol (Dishman, NL), Distearoylphosphatidylcholine (DSPC, Avanti Polar Lipids, Alabama, US), and 1,2-dipalmitoyl-rac-glycero-3-methylpolyoxyethylene glycol-2000 (DPG-PEG2000, NOF America Corporation, New York, US) were dissolved in anhydrous ethanol at the relative lipid molar ratios of 49/39/10/2 mol %.

mRNA and lipid solutions were mixed using a NanoAssemblr Ignite microfluidic mixing device and an NxGen mixing cartridge from Precision Nanosystems Inc. (British Columbia, CA). Briefly, the two solutions were mixed at a 3:1 v/v ratio of mRNA solution to lipid solution at a total flow rate of 9 mL/min. Following mixing, ethanol removal and buffer exchange were performed on the resulting LNP suspension using a discontinuous diafiltration process via 100 kD Regenerated Cellulose centrifugal UF devices (EMD Millipore, Massachusetts, US), and centrifuging at 500×g. LNPs were recovered from the centrifugal UF device and stored at 4° C. for short-term use. Otherwise, LNP solutions were spiked with a 49 wt % sucrose stock solution to reach a final sucrose concentration of 9.8 wt %. LNPs were then frozen and kept at −80° C. for long-term storage.

CAR Design and mRNA Production

The anti-human CD19 (FMC63) and CD22 (m971) antibody VH and VL sequences were derived from public sources (CD19-FMC63 PMID1725979; CD22-m971 PMID20065646) and encoded into a second-generation CAR cassette as scFvs in the VH/VL orientation with a (G4S) 3 linker between the variable heavy and light chains. The 22-V1, 22-V4, 22-V6, 22-V8, and 22-V9 scFvs were derived from an internal antibody campaign and encoded into a second-generation CAR cassette in the VH/VL orientation with a (G4S) 4 linker between the variable heavy and light chains. The second-generation CARs incorporated a CD28 hinge, a CD28 transmembrane domain, and CD28 and CD3z intracellular signaling domains unless otherwise specified.

For the CAR screening, the highest affinity binding scFv antibodies; 22-V1, 22-V4, 22-V6, 22-V8, 22-V9, and m971, were incorporated into CAR cassettes consisting of a CD28-derived hinge of 40 amino acids, a CD8alpha-derived hinge of 26 amino acids (short), a CD8alpha-derived hinge of 46 amino acids (intermediate), a CD8alpha-derived hinge of 66 amino acids (long), respectively, with a CD28 co-stimulatory domain and a CD3z intracellular signaling domain. Additionally, a variant of 22-V4 was designed to contain a 4-1BB-derived co-stimulatory domain, replacing the CD28-derived co-stimulatory domain. mRNA was produced in-house. Briefly, mRNA encoding a second generation CD19 or CD22 CAR with a CD28 or 4-1BB co-stimulatory domain and a CD3zeta signaling domain was in vitro-transcribed using SP6 polymerase, poly-A tail was added enzymatically to the length of 100-120 residues, and 5′ capped (Cap1).

mRNA RP-HPLC Purification

mRNA was purified using an adapted reverse phase HPLC (RP-HPLC) method from Karikó et al. [34]. Briefly, mRNA was purified by RP-HPLC using a Phenomenex Clarity Oligo-RP column maintained at 60° C. with a water/acetonitrile/triethylammonium acetate gradient. The resulting fractions were buffer exchanged into 1 mM sodium citrate, pH 6.5, using 50K MWCO centrifugal filters.

### Targeting Moiety Design and Production

For the CD3- and CD8-targeting Fabs, in both the human heavy and light chain constant domains, the native interchain disulfide-forming cysteines were mutated to serine. Additionally, mutations to bury the disulfide were introduced into both the heavy and light chains to enable a stabilized disulfide linkage while avoiding nonproductive maleimide-PEG-DSPE conjugation. At the end of the human CH₁, the human IgG1 hinge was used up to the first natural cysteine followed by a 6-his tag (EPKSSDKTHTCHHHHHH), enabling conjugation to maleimide-PEG-DSPE and purification by IMAC. Variable heavy and light chain amino acid sequences for anti-human CD3 Fab (Clone: SP34-2) and CD8 Fab (Clone: TRX2) were derived from public sources. The Fab control (mutOKT8) was derived from the OKT8 clone and the CDRs were mutated to abolish binding to CD8. The anti-CD8 VHH (Nb8) was derived from an internal campaign. Fabs and VHHs were produced in HEK, purified by IMAC, and formulated into PBS by Biointron (Metuchen, NJ).

### Sequence Optimization of Anti-CD8 VHH

Humanization of the anti-CD8 VHH, Nb8, was performed internally. Briefly, the protein sequence of Nb8 was optimized, involving the mutagenesis of one or more specific amino acid residues to make the binder more human-like, to reduce its binding by pre-existing antibodies, and to improve its long-term stability. This resulted in the three sequence-optimized variants: Nb8-H1, Nb8-H2, and Nb8-H3.

### Targeting Moiety Conjugation and Post-Insertion

The conjugation reaction was initiated by the addition of a 10 mg/mL micellar suspension of DSPE-PEG-maleimide (Avanti Polar Lipids, Alabama, US) and 30 mg/mL DSPE-PEG-OCH3 (Avanti Polar Lipids, Alabama, US) (1:1 to 1:3 weight ratio is used depending on protein) in oxygen-free pH 5.7 citrate buffer (1 mM Citrate). The protein solution was concentrated to 3-4 mg/mL using a 10 kDa Regenerated Cellulose Membrane (Amicon, EMND Millipore) and subsequently buffer-exchanged in oxygen-free pH 7 phosphate buffer using a 40 kDa SEC column (Zeba, Thermo Fisher). The conjugation reaction was carried out using 2-4 mg/mL protein and a 3.5 molar excess of maleimide at 37° C. for 2 hours followed by incubation at room temperature for an additional 12-16 hours. The production of the resulting conjugate was monitored by HPLC, and the reaction was quenched in 2 mM cysteine. The resulting conjugates (DSPE-PEG (2k)-Fab DSPE-PEG (2k)-VHH) were isolated using a 100 kDa Regenerated Cellulose Membrane (Amicon, EMD Millipore) using pH 7.4 HEPES buffer saline (25 mM HEPES, 150 mM NaCl) buffer and stored at 4° C. prior to use. After quenching, the final micelle composition consisted of a mixture of DSPE-PEG-Fab or DSPE-PEG-VHH, DSPE-PEG-maleimide (cysteine terminated), and DSPE-PEG-OCH3. DSPE-PEG (2k)-Fab or DSPE-PEG (2k)-VHH conjugate was then combined with base LNPs and placed in a ThermoMixer (Eppendorf) at 37° C. at 300 rpm for 4 hours, followed by storage at 4° C. until use.

### Cell Lines

The CD19- and CD22-positive cell lines, Raji, Daudi, Nalm6, Reh, JVM-2, HT, Namalwa, EB-1, Toledo, Su-DHL-6, and Su-DHL-8, and the CD19- and CD22-negative cell line, K562, were purchased from ATCC and were maintained according to the manufacturer's recommendations. CRISPR/Cas9 was used to create CD22 KO Raji and Nalm6 cell lines, respectively. Briefly, CD22 gRNA and CAS9 mRNA were electroporated into WT Raji and WT Nalm6 cells, respectively, followed by a CD22-negative sort using a Sony SH800.

### CD3- and CD8-Dependent Uptake in Isolated T-Cells In Vitro

CD3+ and CD8+ T-cells were isolated from human PBMCs using human CD3 or CD8 T-cell negative magnetic isolation kits (StemCell Technologies). Prior to LNP treatment, isolated T-cells were seeded into 96-well round bottom plates at a concentration of 1×10⁶ cells/mL (100K cells per well) in T-cell media (RPMI-1640, 10% FBS, and 50 ng/mL recombinant human IL-2). Cells were then treated by adding targeted LNPs encapsulating mRNA to the cells followed by mixing by pipetting and incubating at 37° C.

Cells were washed in phosphate buffer saline (PBS) containing 0.5% BSA and 0.1% NaN3 (FACS buffer) for cytometry analysis. After washing, cells were stained with CD4-, CD3-, and CD8-antibodies. CD19 CAR-expression was detected using a recombinant CD19 Ag-FITC conjugate (ACROBiosystems). CD22 CAR-expression was detected using a primary non-fluorescent CD22 Ag (ACROBiosystems) followed by a secondary stain using an anti-CD22-APC antibody (Clone: 4KB128, Invitrogen) or by staining with an anti-G4S antibody (Cell Signaling Technology). All stains were performed at 4° C. for 30 minutes prior to washing and resuspending in FACS buffer. Stained cells were acquired by flow cytometry on a Symphony (Becton Dickinson) running FACSDiva software, a Penteon Novocyte (Agilent), or a Cytek Aurora (Cytek Biosciences), and further analyzed in FlowJo (BD) or OMIQ (Dotmatics). Dead cells were excluded from the analysis by using eFluor780 fixable viability dye (Thermo Fisher).

### LNP Transfection in Human Whole Blood

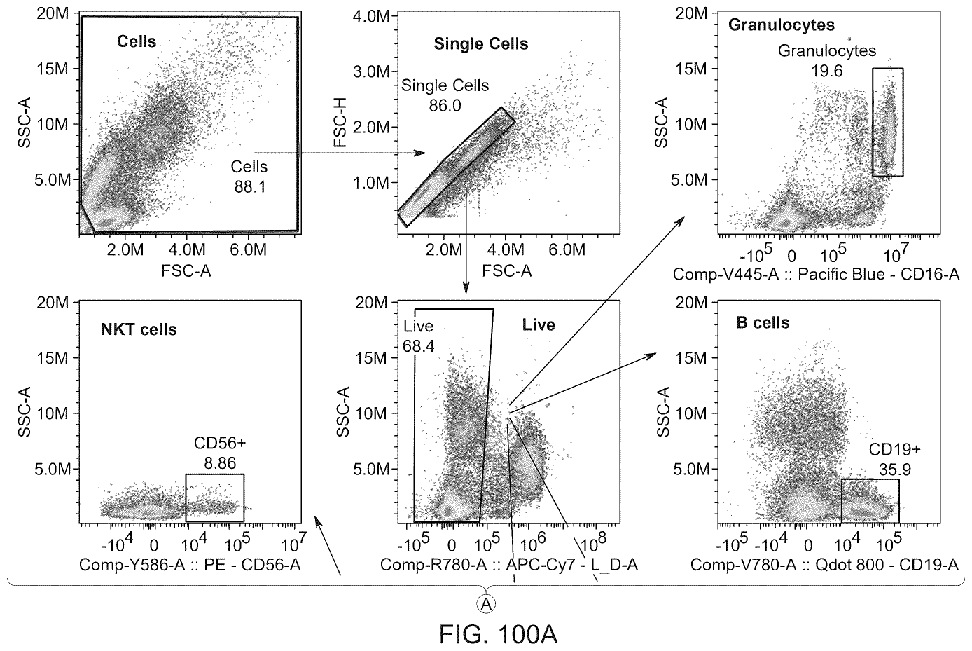

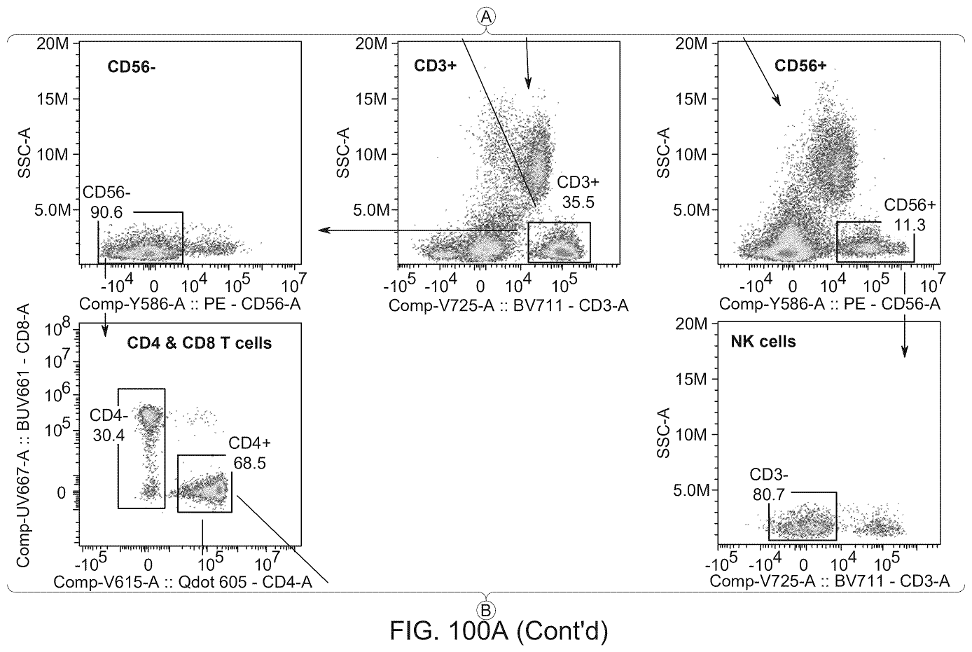

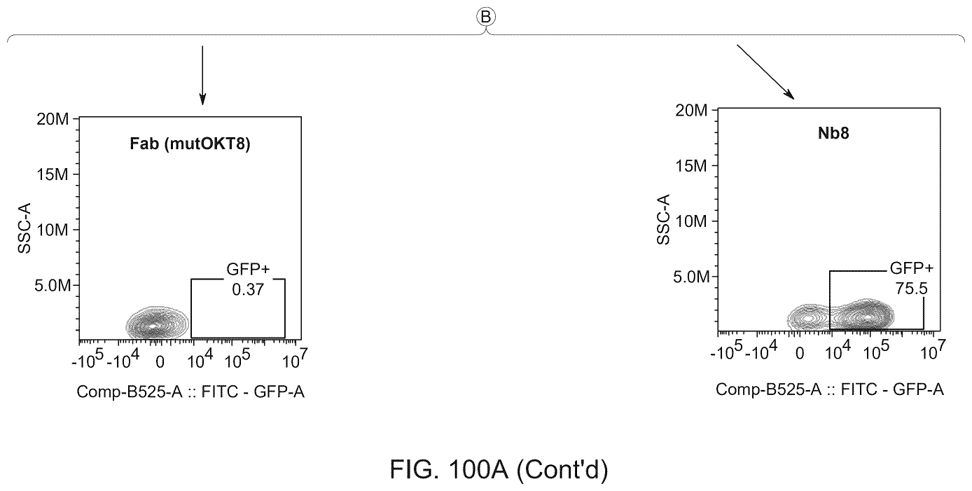

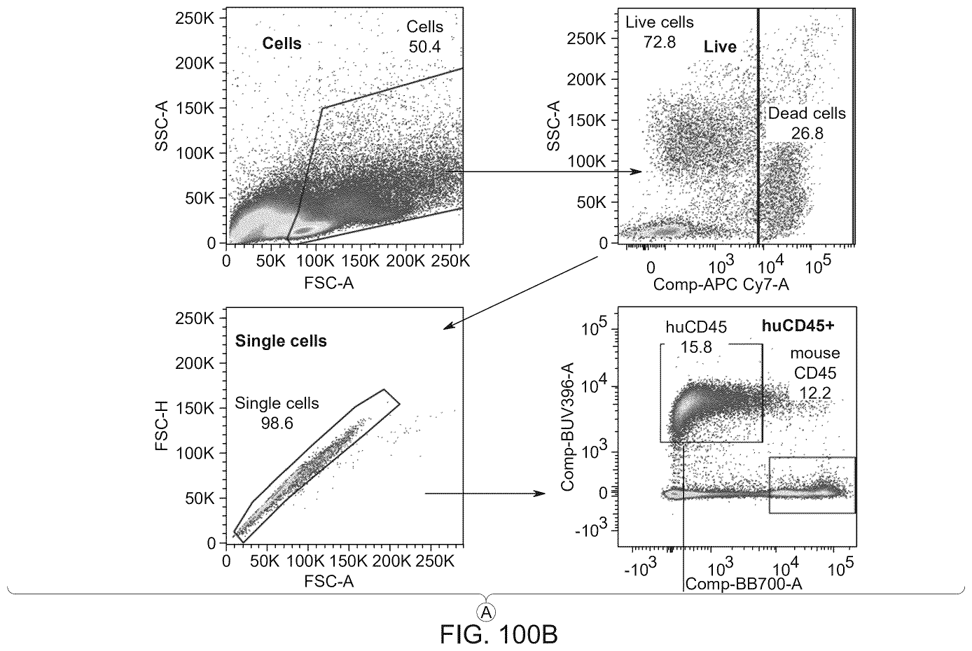

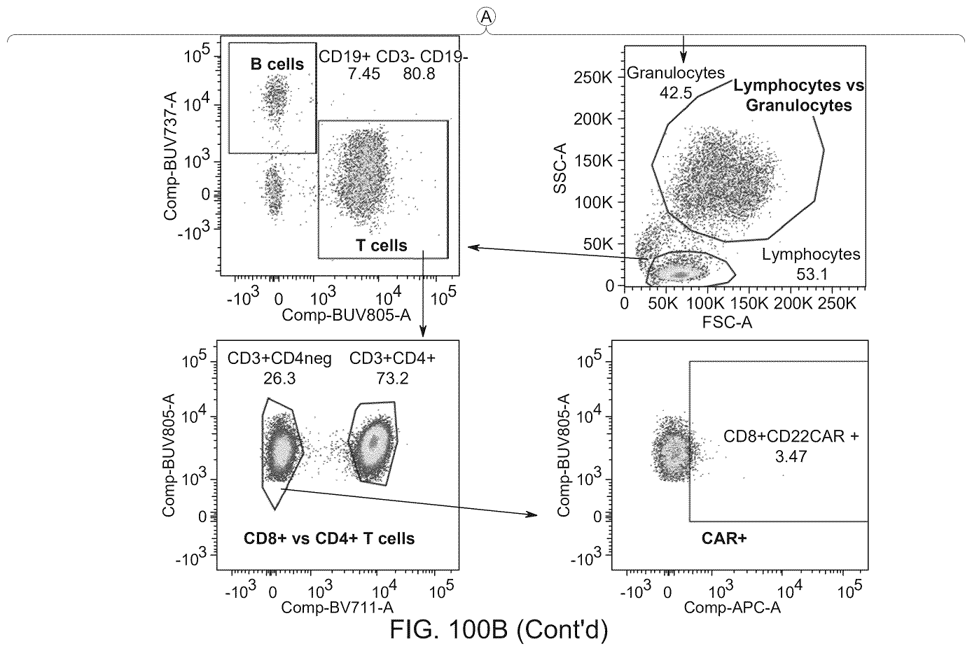

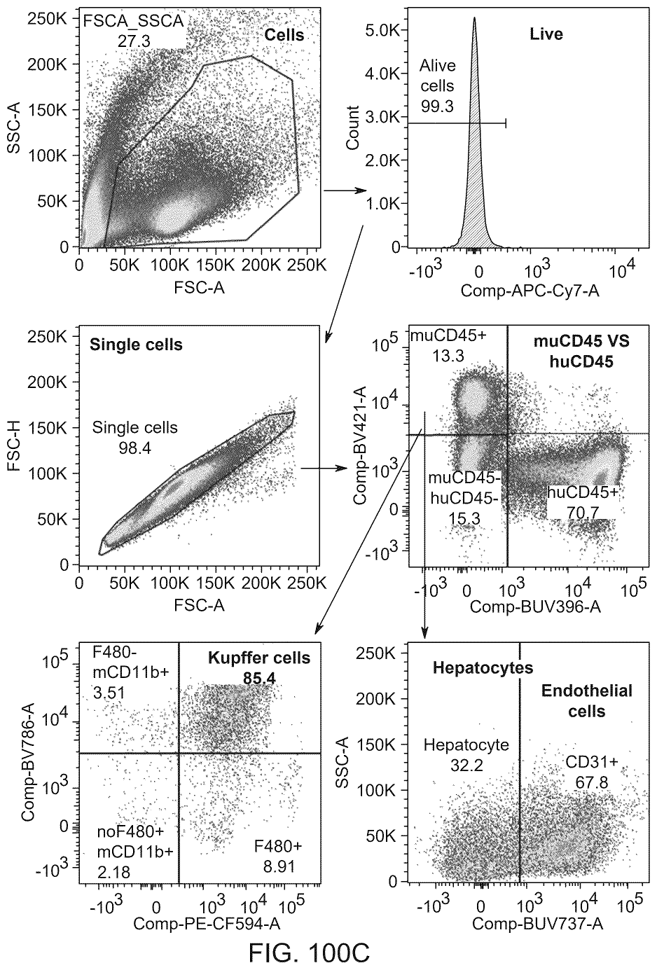

After collection from eight healthy human donors into sodium heparin collection tubes (BD Biosciences), 100 μL fresh human whole blood was seeded into a 96-well round bottom plate and transfected with targeted LNPs. Red blood cells were lysed 24 hours post-transfection using Versalyse lysis buffer (Beckman Coulter) and immune cells were Fc blocked and stained with CD14-BUV495 (BD Biosciences), CD8-BUV661 (BD Biosciences), CD16-BV421 (BD Biosciences), CD4-BV605 (BD Biosciences), CD3-BV711 (BD Biosciences), CD19-BV785 (Biolegend), CD56-PE (Biolegend), CD15-PE/Cy5 (Biolegend). Dead cells were excluded from analysis by using eFluor780 fixable viability dye (Thermo Fisher). Granulocytes were gated based on SSC and CD16. Total lymphocytes were gated based on SSC and the following subsets were defined: CD4+ T-cells were defined as CD3+, CD56−, and CD4+, CD8+ T-cells were defined as CD3+, CD56−, and CD4− due to steric hindrance and internalization mediated by the CD8− targeted LNPs, NKT cells were defined as CD3+ and CD56+, NK cells were defined as CD3− and CD56+, and B-cells were defined as CD19+. The GFP expression was evaluated after 24 hours of transfection in all the abovementioned cell subsets. The gating strategy is shown in FIG. 100A.

Cytokine Profiling with MSD

Cytokine secretion in vitro was analyzed by MSD (Meso Scale Diagnostics). Supernatants were pulled from T-cell culture at the 24-hour time point and a custom U-PLEX assay was used for detection of human TNF-alpha, IFN-gamma, and IL-10 following the manufacturer's instructions. For cytotoxicity assays, supernatants were pulled 48 hours after co-culture and a custom U-PLEX assay was used for detection of human TNF-alpha, IFN-gamma, IL-10, and Granzyme B following the manufacturer's instructions. For in vivo analysis, blood was drawn at the respective time points into 300 μL Microvette 100 EDTA tubes and centrifuged at 500×g for 10 minutes. Plasma was then collected and analyzed using a custom U-PLEX assay for detection of human TNF-alpha, IFN-gamma, and IL-10 following the manufacturer's instructions.

### Flow Cytometry-Based Cytotoxicity Assay

Raji WT, Raji CD22KO, Nalm6 WT, Nalm6 CD22KO, Daudi WT, JVM-2 WT, Reh WT, HT WT, Namalwa WT, EB-1 WT, Toledo WT, Su-DHL-6 WT, Su-DHL-8 WT, and K562 WT cells were stained with Cell Trace Violet proliferation dye (CTV) (Thermo Fisher) following the manufacturer's protocol. Briefly, target cells were incubated with CTV for 20 minutes at room temperature prior to washing and co-culturing. CD8⁺ T-cells were isolated and transfected as described above and washed after 2 hours of incubation to remove unbound LNPs. Transfected CD8⁺ T-cells were then co-cultured with CTV-stained target cells at varying effector-to-target cell ratios in T-cell media (RPMI-1640, 10% FBS, and 50 ng/mL recombinant human IL-2) in a 96-well flat-bottom plate for 48 hours. After 48 hours of co-culture, cells were stained with eFluor780 fixable viability dye (Thermo Fisher) to assess the level of cytotoxicity. Cells were washed twice in FACS buffer and acquired by flow cytometry on a Penteon Novocyte (Agilent) and further analyzed in FlowJo, where dead target cells were defined as CTV⁺ and Live/Dead eFluor780⁺ cells.

### Modular IMmune In-Vitro Construct (MIMIC®) Technology

A MIMIC® CRA (Cytokine release assay) was used to evaluate cytokine secretion induced by various formulations of LNP constructs. MIMIC® CRA is a 3D endothelial cell-based assay that utilizes fresh human whole blood donations. Red blood cell (RBC)-depleted leucocytes along with autologous plasma were used in this assay. The assay is designed for analysis of acute immune responses generated by intravenously delivered drugs. The assay was performed as described elsewhere [36-38]. Briefly, fresh leukocytes (RBC-depleted whole blood cells) were acquired from blood donations from healthy donors who provided informed consent and were enrolled in the Sanofi Pasteur VaxDesign donor program (Chesapeake Research Review, Inc., Columbia, MD., protocol 0906009). From each volunteer's sample, whole blood leukocytes and their corresponding autologous platelet-poor plasma were separated by centrifugation at 2100 rpm. The separated plasma was spun down and cleared through a 0.2-μm filter (polyetherslfone; Nalgene) to remove platelets and platelet particles. To remove red blood cells, the leukocyte pellets were mixed with a sterile solution of 5% weight by volume (w/v) dextran (Sigma) to allow sedimentation of the erythrocytes. Thereafter, supernatants were collected and washed two times with Dulbecco's phosphate buffer saline (Lonza). Then, white cells were counted using trypan blue exclusion staining.

To initiate the MIMIC® CRA assay, 96-well plates were coated with a collagen solution (Advanced BioMatrix, Inc.) to form a thick cushion on the bottom of the well (day 0). An endothelial cell line (EA. hy926; ATCC) was seeded onto the collagen cushions in a media with serum one day later (day 1). After growth until confluency (about four days), the media was changed to a non-serum media. Next day (day 5) fresh reconstituted leukocytes (prepared as described above) from healthy donors were applied to the constructs along with different LNP formulations diluted in autologous plasma at the dose of 10 and 1 μg/ml. CD28SA (Ancell corporation), Poly IC (Invivogen), and R848 (Invivogen), respectively, were used as positive controls at a dose of 10 μg/ml. After 20-22 hours, culture supernatants were collected and analyzed for cytokine and chemokine production using multiplex arrays.

Culture supernatants from MIMIC® CRA were harvested after 20-22 hours of treatment with test articles and controls. Supernatants were analyzed using EMD Millipore's MILLIPLEX® MAP Human Cytokine and Chemokine Magnetic Bead Panel. This kit was used for the quantification of the following human cytokines and chemokines: GM-CSF, IFN-α1, IFN-α2, IFN-γ, IL-10, IL-12p40, IL-12p70, IL-1B, IL-2, IL-4, IL-5, IL-6, IL-8, IP-10, MCP-1, MIP-1β, RANTES, TNF-α. The manufacturer's protocol was followed, except that the standard was diluted at 1:3 instead of 1:5. Analyte concentrations were calculated based on relevant standard curves using the Bio-Plex manager software version 6.2. For running acceptance criteria, the lower limit of quantification (LLOQ) and the upper limit of quantification (ULOQ) for each analyte was established based on the percent recovery (Observed/Expected*100) of each point against the 5-parameter logistic curve fit of the standard values. A recovery range of 80-120% was considered acceptable, such that values falling within this range defined the lower and upper bounds of the standard curve. The raw data file was reviewed for bead counts; a data point was considered valid when a minimum of 35 beads were counted per region.

All data was exported into excel databases. Out-of-range high values (values greater than the highest point of the curve) were replaced by the highest point on the standard curve. Out-of-range low values were replaced with ½ of the LLOQ.

### Incucyte Cytotoxicity Assay

The Incucyte NucLight Red Lentivirus reagent (Sartorius) was used to transduce Nalm6 WT cells following the manufacturer's protocol. Transduced NucLight Red positive cells were selected for using puromycin and >95% purity was confirmed by flow cytometry prior to co-culture assays. CD8+ T-cells were isolated and transfected as described above. Unbound LNPs were removed by washing in PBS 2 hours post-transfection. Transfected T-cells were then co-cultured with NucLight Red Lentivirus transduced Nalm6 cells in T-cell media (RPMI-1640, 10% FBS and 50 ng/mL recombinant human IL-2) in a 96-well flat-bottom plate. Cancer cell killing was monitored in the SX5 Incucyte every 2 hours. Re-challenge of the T-cells was performed by gently removing the supernatant and replacing it with NucLight Red Lentivirus transduced Nalm6 cells in T-cell media. Re-transfection was performed by gently removing the supernatant and replacing it with T-cell media containing fresh LNPs. The level of cytotoxicity was quantified by normalizing the count of red cells to the initial time point.

### In Vivo CAR Reprogramming in Humanized NSG Mice

For reprogramming T-cells in vivo, 0.3 mg/kg mRNA encapsulated in LNPs was administered intravenously (IV) through the tail vein of 10-week-old female NSG mice engrafted with 10 million human PBMCs 10 days prior. Blood was collected into EDTA-coated collection tubes and red blood cells (RBCs) were lysed using Versalyse (Beckman Coulter). Immune cells were Fc blocked and stained with human CD8-BV421 (BD Biosciences), human CD4-BV711 (BD Biosciences), human CD3-BUV805 (BD Biosciences), human CD20-BUV737 (BD Biosciences), human CD45-BUV395 (BD Biosciences), murine CD45-BB700 (BD Biosciences), CAR-APC (internal). Dead cells were excluded from analysis by using eFluor780 fixable viability dye (Thermo Fisher). Granulocytes were gated based on FSC/SSC. Lymphocytes were gated based on FSC/SSC and the following subsets were defined: human CD4+ T-cells were defined as huCD45+CD3+CD4+, human CD8+ T-cells were defined as huCD45+CD3+CD4− due to steric hindrance and internalization mediated by the CD8-targeted LNPs, and human B-cells were defined as huCD45+CD19+. The CAR expression was evaluated after 24 hours of transfection in all the abovementioned cell subsets. The gating strategy is shown in FIG. 100B. Cells were washed twice in FACS buffer and acquired by flow cytometry on a Symphony (Becton Dickinson) running FACSDiva software and further analyzed in FlowJo. Spleen, liver, lung, and bone marrow were first excised, weighed, and processed to single-cell suspensions before staining and acquiring by flow cytometry as described above.

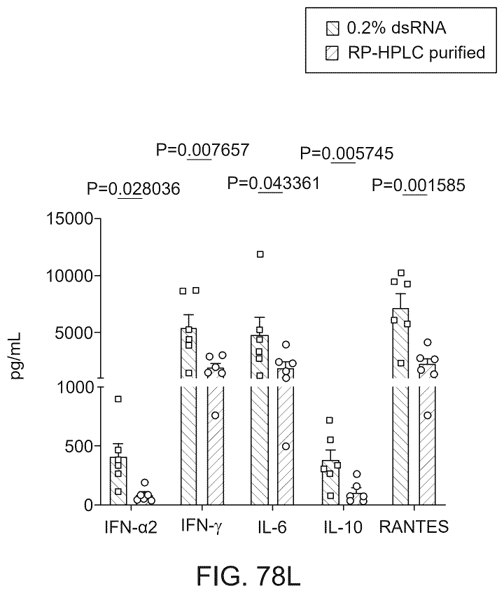

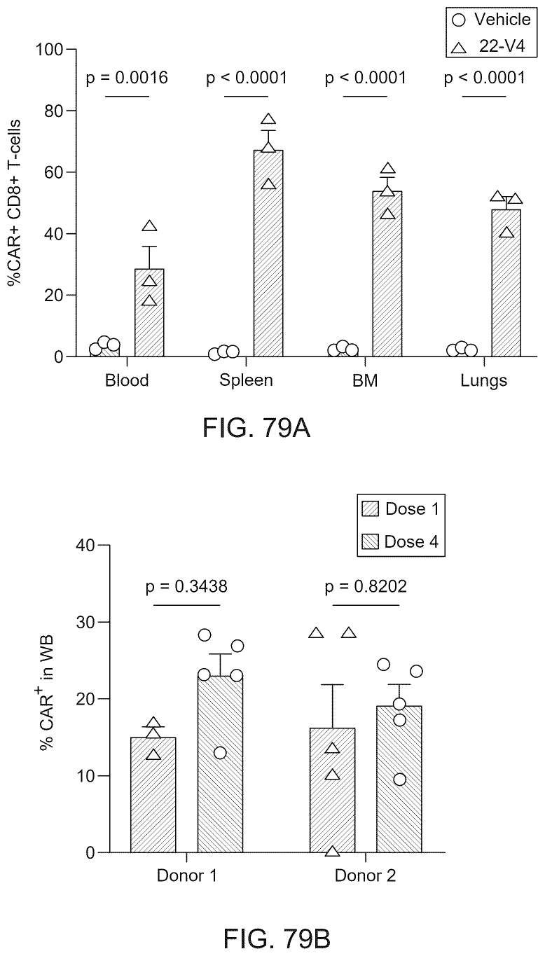

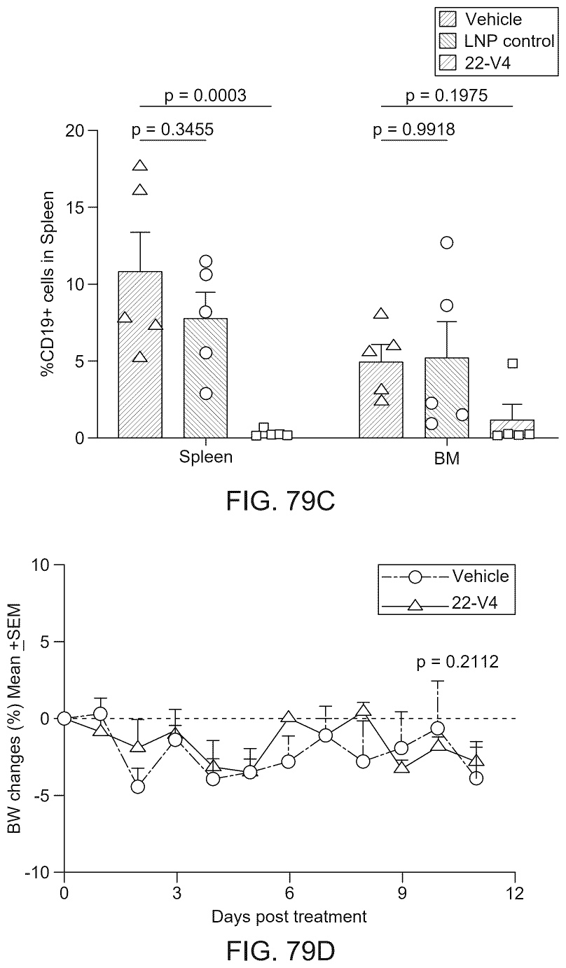

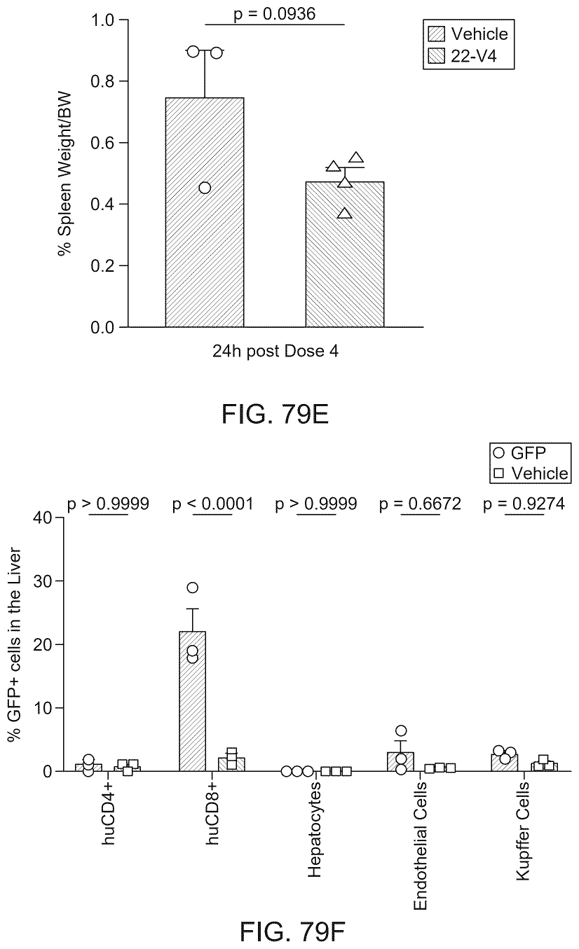

For the myeloid flow cytometry panel of the liver (FIG. 79F), cells were Fc blocked and stained with human CD45-BUV395 (BD Biosciences), murine CD45-BV421 (BD Biosciences), murine CD11b-BV786 (BD Biosciences), murine F4/80-PE-CF594 (BD Biosciences), murine CD31-BUV737 (BD Biosciences). Dead cells were excluded from analysis by using eFluor780 fixable viability dye (Thermo Fisher). The following subsets were defined: murine hepatocytes were defined as huCD45-muCD45-muCD31−, murine endothelial cells were defined as huCD45-muCD45-muCD31+, and murine Kupffer cells were defined as muCD45+muCD11b+muF4/80+. The gating strategy is shown in FIG. 100C. Human CD4 and CD8 T-cells were stained and defined as described above but murine CD45-BB700 was excluded to allow for GFP expression and detection in the channel.

### In Vivo Efficacy in Hematological Malignancy Model

NSG MHC I/II knockout mice were IV injected with 2×10⁶ Nalm6-Luc cells on day 0 and with naïve human PBMCs on day 6. 0.3 mg/kg mRNA-LNPs was administered by IV injection on days 7, 11, 14, 18, and 21. To monitor tumor progression, mice were imaged by bioluminescence imaging on days 6, 11, 14, 18, 21, and 26. Briefly, mice were i.p. injected with 150 μg/g body weight D-luciferin (PerkinElmer) and then imaged 10 minutes later using an IVIS (PerkinElmer) and while being under anesthesia by isoflurane.

### Immunohistochemistry

Formalin-fixed samples of livers and spleens collected from mice on day 26 of the efficacy study were processed and paraffin-embedded. Staining for immunohistochemistry (IHC) was done using a mouse anti-human monoclonal antibody (Abcam; BLCAM/1795 clone) with specificity for the human B-lymphocyte marker, CD22. All slides were subjected to histopathology evaluation. Slides were blindly evaluated by a board-certified veterinary pathologist. Quantitative analysis for hCD22 IHC was performed on whole slide digital images within the HALO Indica Labs image analysis software platform with the Cytonuclear v2 algorithm. Individual mouse scores are shown in Supplemental Tables 10 and 11.

### Study Approval

All animal procedures were performed in accordance with the National Institutes of Health Guide for the Care and Use of Laboratory Animals and were approved by the Sanofi Institutional Animal Care and Use Committee (AICUC). Following drug treatment, mice were observed daily for overall health including general appearance of the fur, mobility, and body weight. The animals were euthanized before they exhibited clinical signs and symptoms to avoid unnecessary pain and discomfort, according to internal ethical animal guidelines.

Human whole blood was collected from healthy adult (>18 years) donors in heparin Vacutainer® tubes under an approved protocol for Sanofi following informed consent prior to the blood draw.

### Statistical Analysis

Statistical differences were assessed as indicated in the figure legends. Statistical analysis was performed using GraphPad Prism. Data are presented as mean values+/−SEM unless otherwise indicated. A p value less than 0.05 was considered to be statistically significant. The number of samples in each experiment is stated in the respective figure legends.

### Results

### Engineering a T-Cell Targeted LNP to Reprogram CAR-T Cells In Vivo

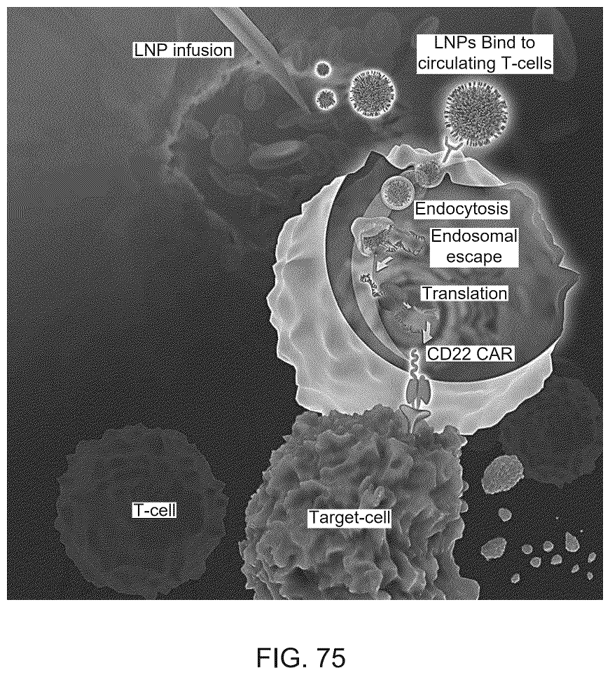

We hypothesized that CAR-encoding mRNA could be encapsulated in LNPs and through an antibody-based targeting moiety directed toward CD8⁺ T-cells mediating functional CAR-expression directly in vivo (FIG. 75). First, LNPs were engineered to balance efficient endosomal escape with specificity towards CD8⁺ T-cells through careful engineering of the composition to increase binding and internalization by CD8⁺ T-cells while reducing nonspecific cell transfection, including both circulating immune cells as well as those present in organs of the mononuclear phagocyte system (MPS), such as liver and spleen. Unlike many LNPs designed to increase uptake in liver hepatocytes for liver-centric disease applications, or nonspecifically by antigen-presenting cells following intramuscular (IM) administration for vaccine applications, our T-cell targeted LNPs were modified to have a larger particle size (>100 nm) to reduce access to hepatocytes through liver fenestrations, a neutral or slightly anionic surface charge to reduce non-specific binding to negatively charged proteoglycans present on the surface of many endothelial cells, and a more stably anchored PEG-lipid to reduce premature dissociation from the LNP and increase circulation lifetimes allowing more time to engage receptors on the surface of T-cells. An N/P ratio was optimized in the range of 4-6 to allow for efficient condensation of the mRNA while reducing excess ionizable cationic lipid (ICL) that can result in “free” liposomal lipid and the risk of increased toxicity.

The final component of the targeted delivery system was the identification of an antibody fragment that could bind and induce internalization specifically by the T-cell population of interest. The timely development of an LNP with all the desired properties required parallel optimization of different facets of the LNP, and then bringing together the individual optimized components with proper bridging at various points in the platform development cycle. A depiction of the lead targeted LNP is shown in FIG. 71. It consists of a novel ICL, a stabilizing phospholipid (DSPC), cholesterol, mPEG-derivatized diacylglycerol (PEG-DPG), a lipid-anchored antibody-PEG conjugate, and a CAR-encoding mRNA.

LNP formulations were formulated and characterized with respect to their biophysical properties, evaluating particle size and polydispersity index (PDI) by dynamic light scattering (DLS), zeta potential, and mRNA encapsulating efficiency. Formulations with preferred properties had a particle size between 90-120 nm, PDI less than 0.2 and preferably less than 0.1, dye-accessible mRNA of <10% (RiboGreen), and zeta potential that was neutral or slightly anionic at the neutral pH found in the circulation, while being highly cationic under acidic conditions, such as those found in endosomes or lysosomes. The apparent pKa of LNP formulations containing various ICLs was evaluated using a TNS assay with a target pKa of 6.5-7.5. ICLs in this pKa range were expected to provide the pH-dependent surface charge switching driven by the titrable amine in the ICL requisite for stability in the circulation while providing for efficient endosomal escape following target-mediated endocytosis.

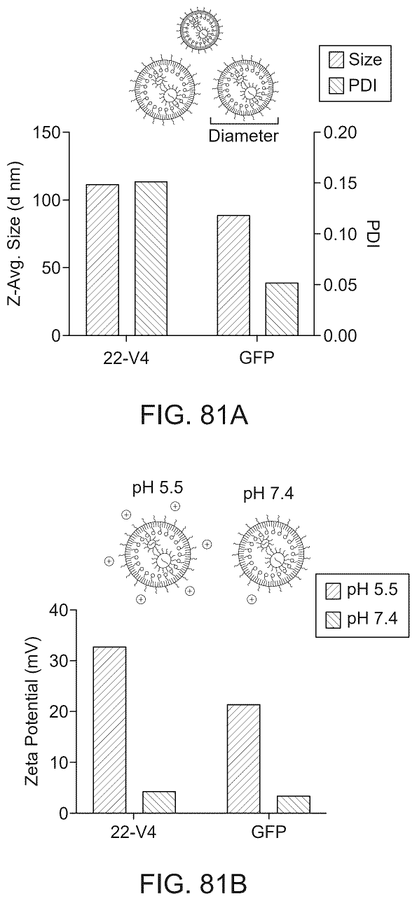

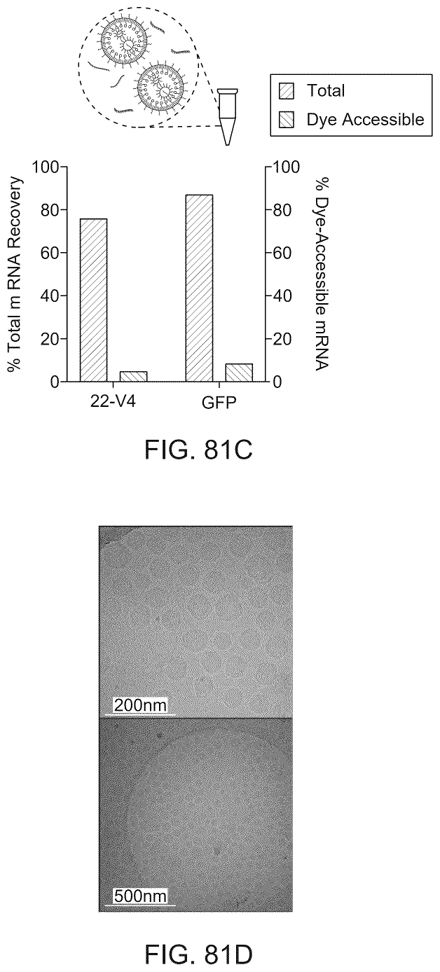

The average diameter of our lead CAR-encoding LNPs employed Lipid 15 as the optimal ICL, showed a particle size of ~100 nm with a PDI of <0.15 (FIG. 75). When evaluating the zeta potential, LNPs based on the ionizable lipid, Lipid 15, and encapsulating CAR-encoding mRNA, were near neutral at physiological pH (pH 7.4) while being positively charged at pH 5.5 with a zeta potential of ~30 mV (FIG. 81B). The same LNP formulation showed to have <10% dye-accessible RNA measured by the RiboGreen assay (FIG. 81C). Finally, Lipid 15 LNPs were characterized by cryogenic electron microscopy (cryo-EM) where the LNPs imaged showed homogeneous morphology and confirmed the size to be ~100 nm as measured by DLS (FIG. 81D).

### Optimized ICL and PEG-Lipid Improves the T-Cell Transfection Efficiency

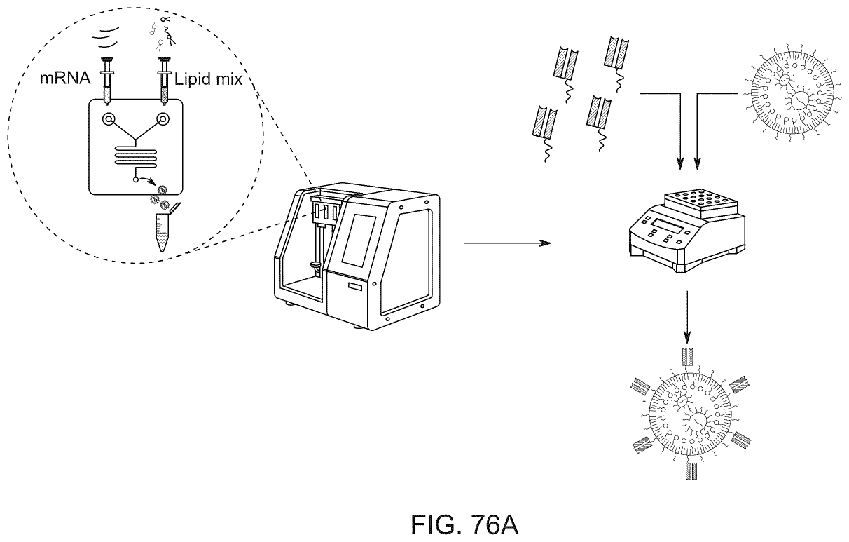

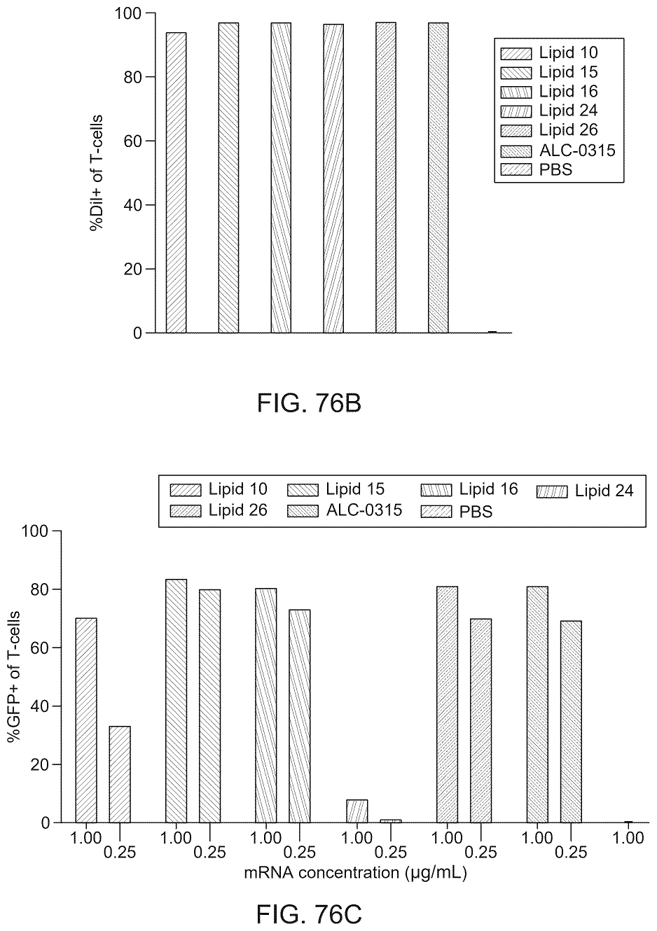

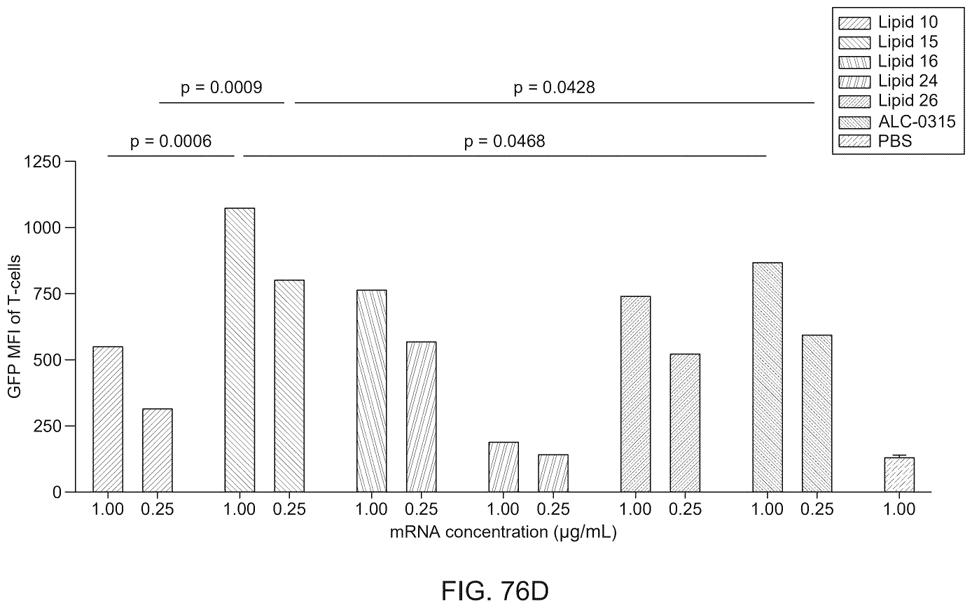

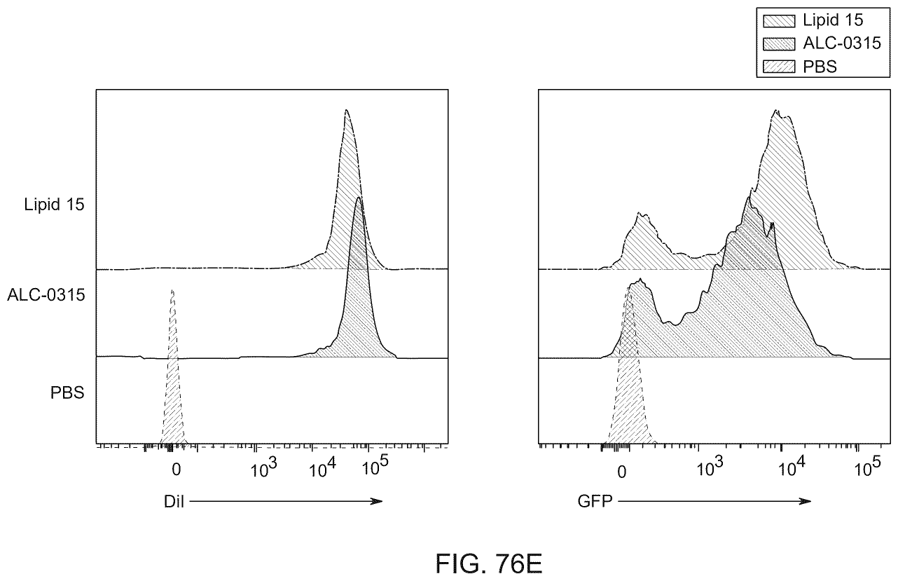

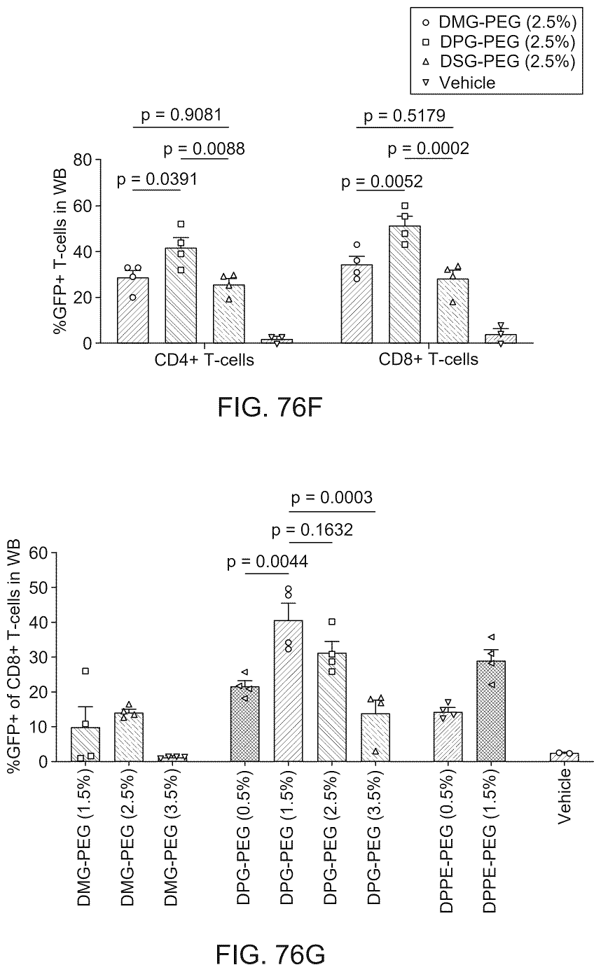

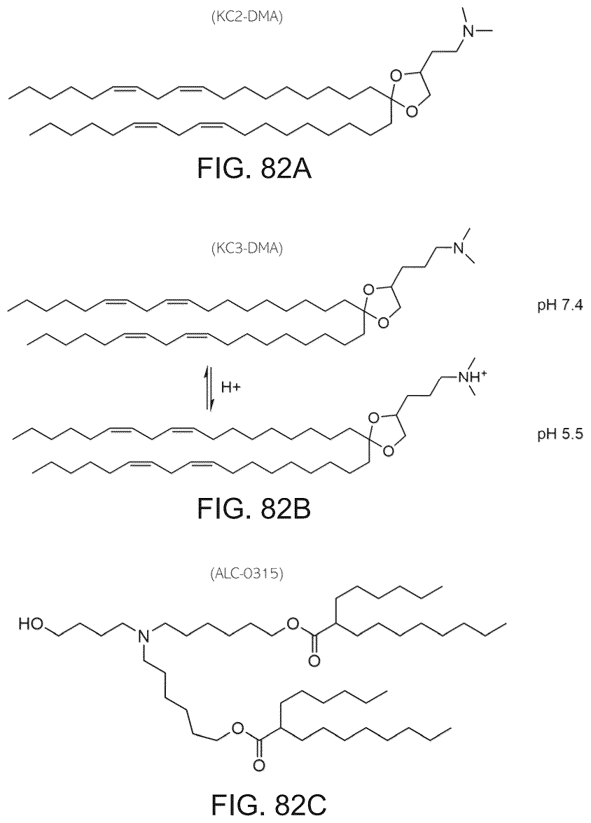

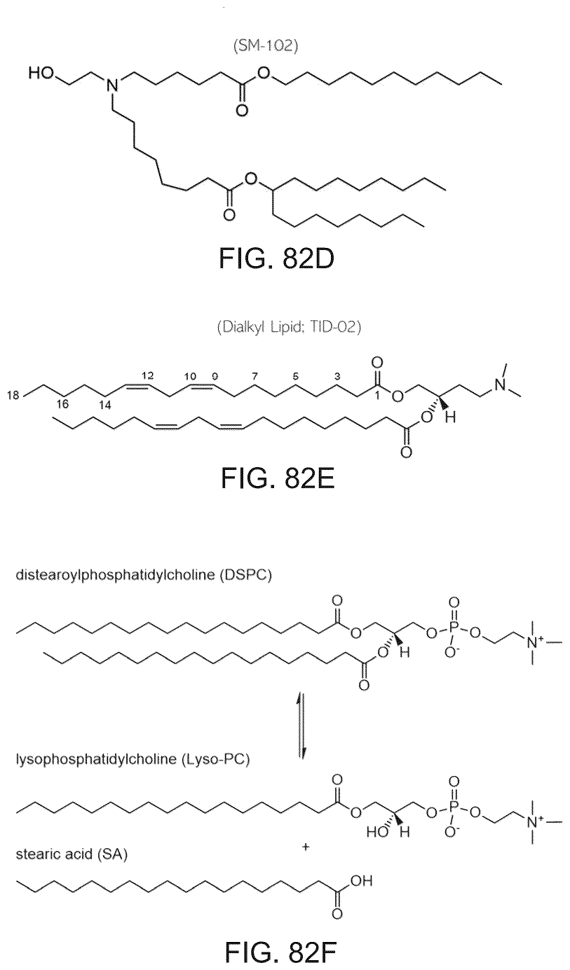

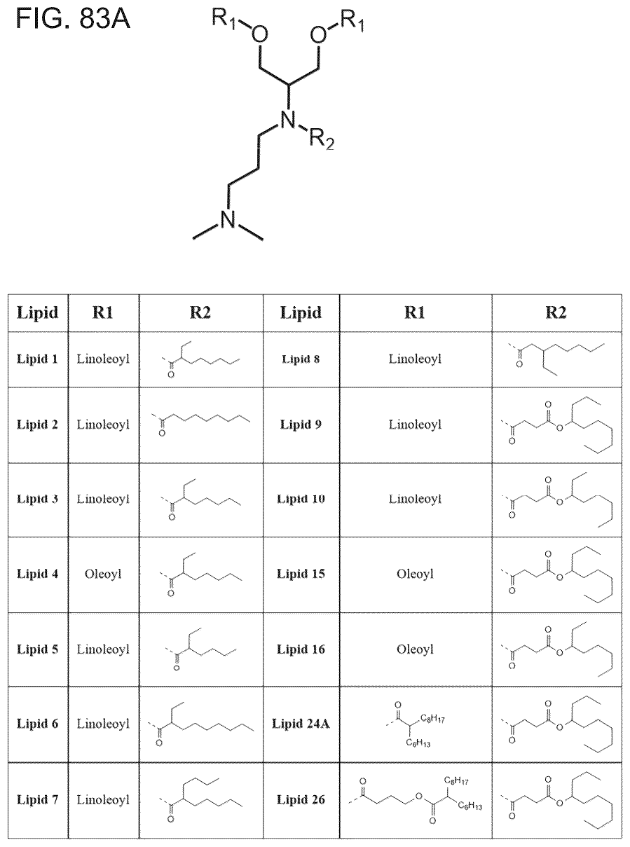

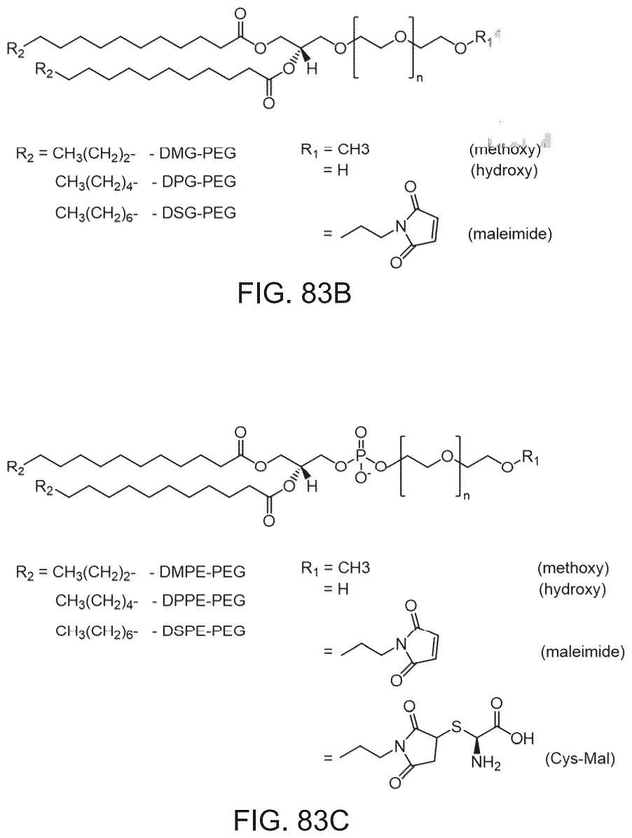

With the ionizable lipid component playing a key role in the transfection efficiency, our initial focus was to design and test different ionizable lipid families that were formulated into LNPs using microfluidic mixing (FIG. 76A). To evaluate the efficiency with which the formulated LNPs could get to and deliver a functional mRNA payload to human T-cells we incorporated a traceable DiI dye into the LNPs and used a reporter mRNA encoding eGFP. The DiI-LNPs encapsulating GFP mRNA were inserted with an anti-CD8 or CD3 antibody conjugate post-formulation and added to primary human T-cells to evaluate the LNP-association and GFP expression by Flow Cytometry and thus compare the different ionizable lipid families (FIG. 76B-FIG. 76E). Initial development of the platform utilized previously identified ICLs (FIG. 82) such as KC2-DMA, ALC-0315, SM-102, KC3-DMA, or a dimethylaminopropyl-dialkyl lipid (TID-02) either as components of the investigational LNPs or as part of comparator LNPs to demonstrate differentiation from LNPs including a novel ICL series shown in FIG. 83.

Molecular design features of this novel lipid family included the use of an aminopropyl diol linker group to bridge the dimethyl aminopropyl ionizable head group to two hydrophobic lipid “tail” groups via degradable ester linkages and a single less hydrophobic N-acyl amide linked “side” group. The two “tail” groups and the single “side” group were chosen to impart an asymmetric structure to the lipid molecule, and the resulting cone-shaped structure was additionally amplified using either degree of unsaturation or by incorporation of branched aliphatic substituents. Furthermore, degradable ester linkages were introduced systematically into both “side” and “tail” groups to impart in vivo biodegradability and enable rapid lipid elimination.

Lipids 1 through 8, the N-acyl “side” group consists of 8 carbon (Lipid 5), 9 carbon (Lipids 2, 3 and 4), 10 carbon (Lipids 1 and 8), or 11 carbon (Lipids 6 and 7)O-acyl substituents in combination with either dioleoyl (lipid 4) or di-linoleoyl (Lipids 1, 2, 3, 5 & 6, 7, 8)O-acyl “tail” groups, thereby spanning a range of “side” group hydrophobic content with mono- and di-unsaturated “tail” groups. Similarly, the three-dimensional structure and steric properties of the “side” were tuned via position and degree of branching, i.e., using linear and branched (Lipid 2 versus Lipids 3 and 4), or α- and β-substitution (Lipid 6 versus 8) of the alkyl groups within the N-acyl “side” group.

In lipids 9, 10, 15, 16, 24A, and 26, a succinate-derived degradable ester linkage was introduced into the “side” group, and the hydrophobicity of the ester moiety was controlled via the choice of succinate ester composition, i.e., less hydrophobic 3-octanol succinate (in Lipid 16) or more hydrophobic 3-decanol succinate (in Lipids 9, 10, 15, 24A and 26). Also, in this sub-set, the lipid “tail” groups comprised of di-linoleoyl (Lipids 9 and 10), di-oleoyl (Lipids 15, 16), di-(8-hydroxydecanoate) (Lipid 24A) or 4-((2-hexyldecanoyl)oxy) butanoate (Lipid 26)O-acyl substituents. These tail group compositions were chosen to evaluate the effect of the degree of lipid unsaturation (Lipids 9, 10, 15, and 16) and branching (Lipids 24A and 26) as these structural parameters are expected to modulate molecular shape (“cone” structure) and lipid melting points and thereby alter the ability of the resulting LNP formulations to enable efficient endosomal membrane disruption and cytosolic delivery of the RNA payload.

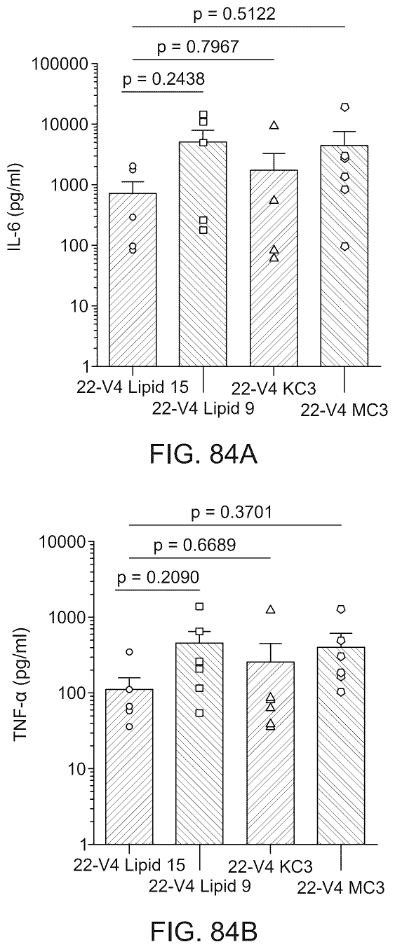

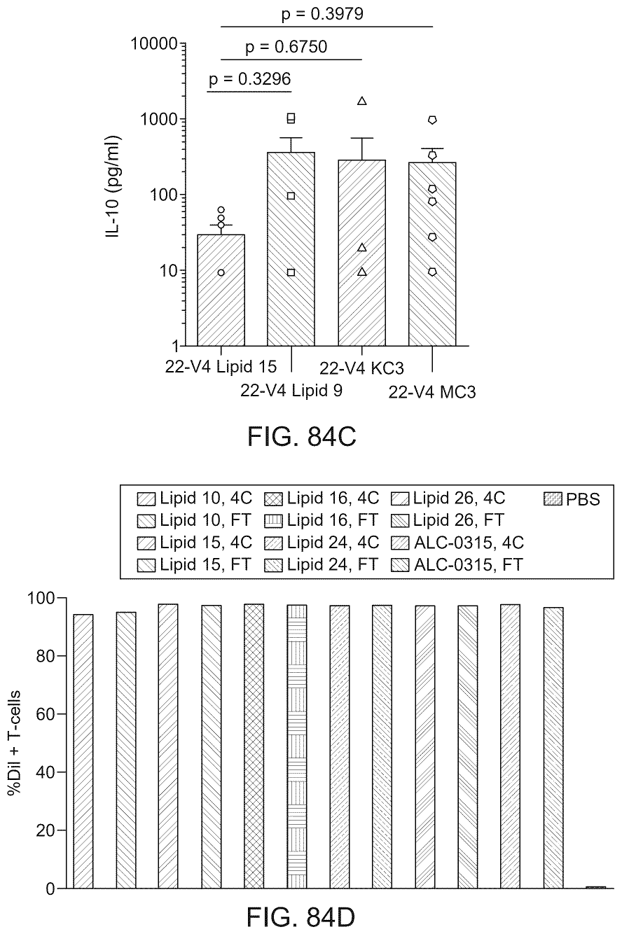

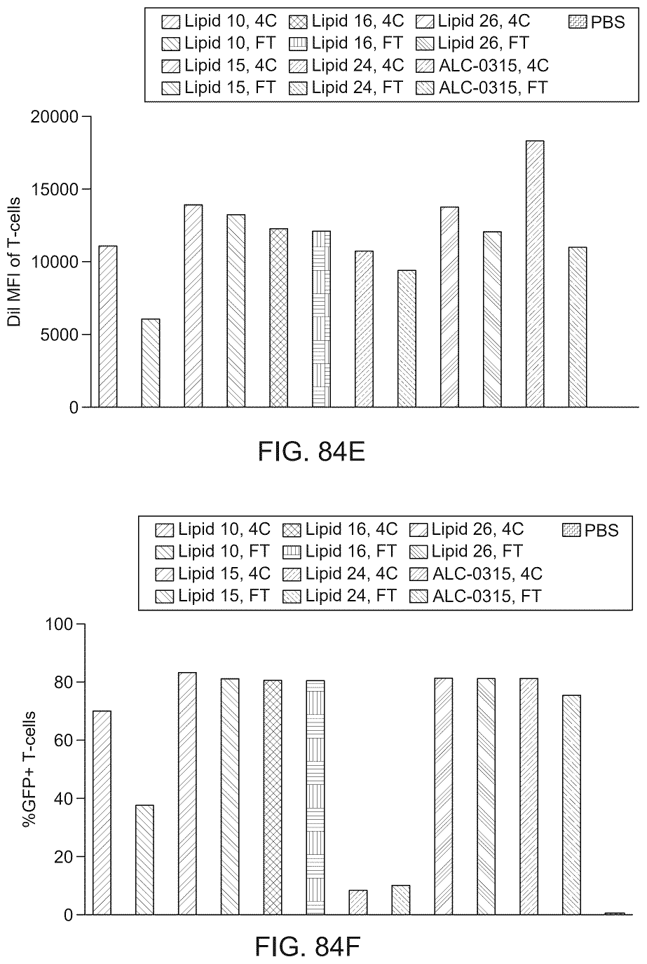

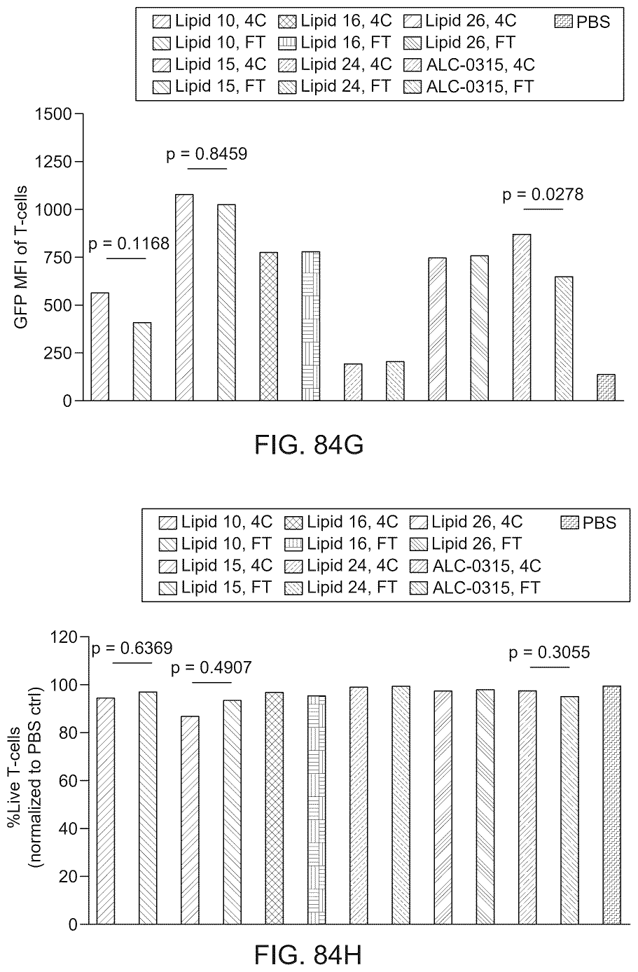

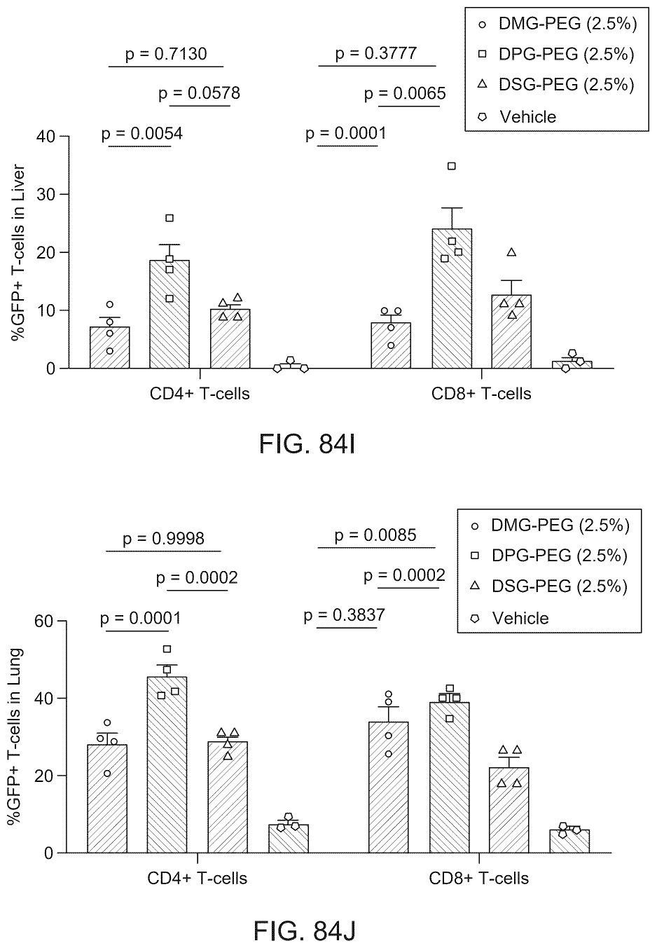

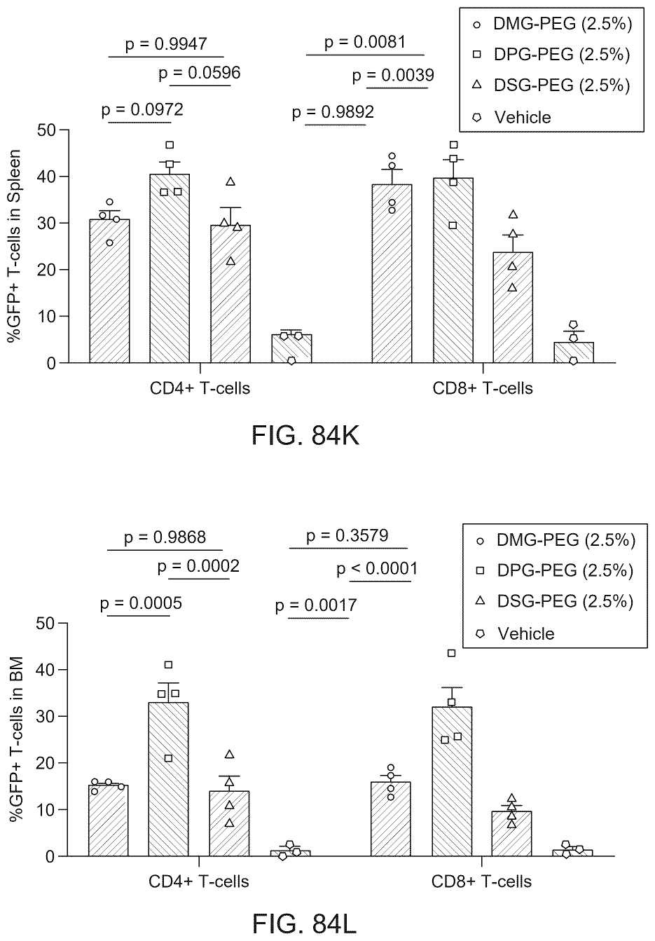

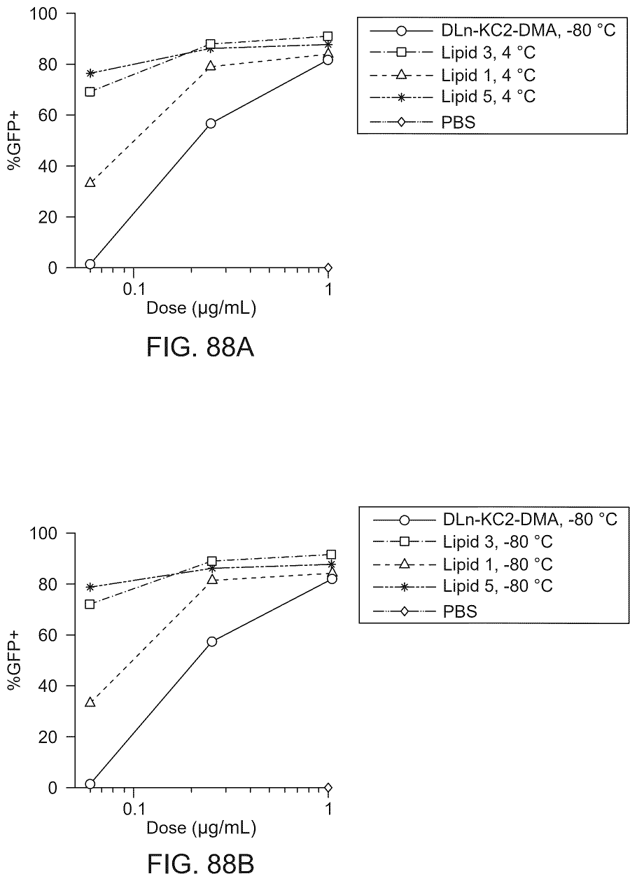

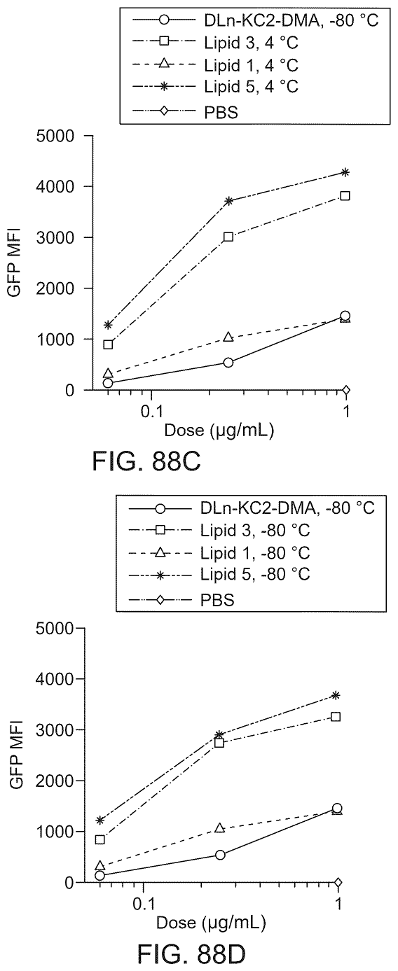

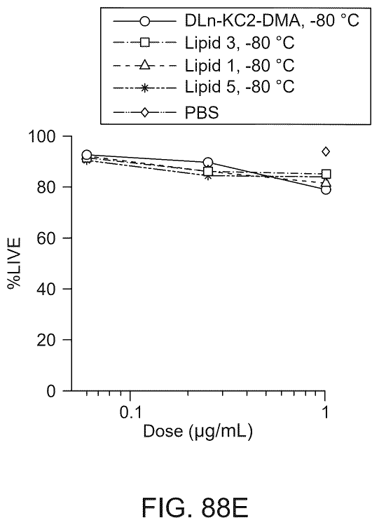

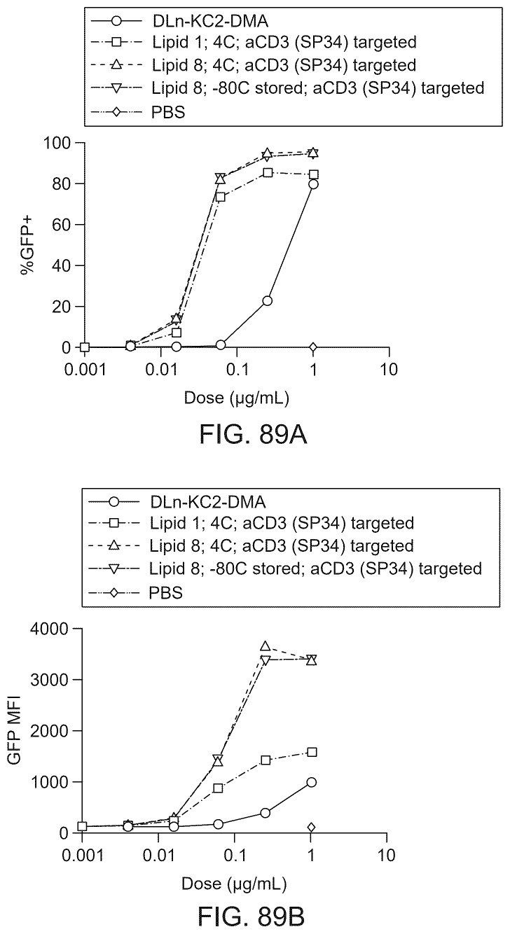

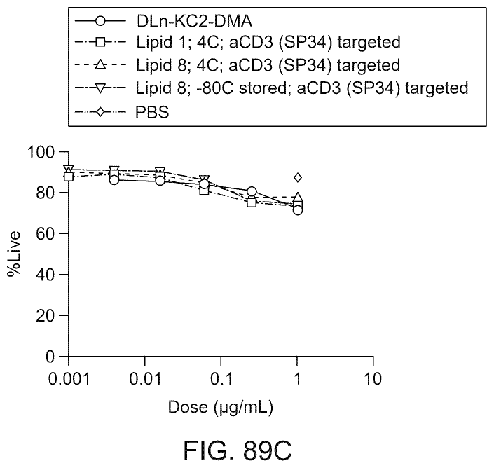

Our lead ICL (Lipid 15) showed superior transfection efficiency, as illustrated by increased GFP MFI values (FIG. 76D), in comparison to other ionizable lipid families that were tested, including the ionizable lipid, ALC-0315, which was used in the SARS-COV-2 vaccine, BNT162b2. Additionally, Lipid 15 LNPs proved to induce lower levels of cytokine secretion compared to other ionizable lipids tested which was evaluated using the MIMIC® assay (FIG. 84A-C). Finally, Lipid 15 LNPs proved to be stable after one freeze-thaw cycle as no significant decrease in T-cell transfection performance was observed based on the GFP MFI values (FIG. 84G). This contrasted with LNPs based on ALC-0315 where a significant drop in T-cell transfection efficiency was observed after a single freeze-thaw cycle. A more detailed evaluation of the structure-activity relationships is provided in the Supplemental Information (FIG. 85-FIG. 93 and Supplemental Tables 1-8). Briefly, a single olefin was preferred over side-chains with dual olefins (e.g. linoleic acid; Lipids 8-10). Biodegradable ester linkages and N-acyl substituents derived from succinate-linked branched aliphatics resulted in improved LNP colloidal stability. Lipids that displayed a strongly positive charge at acidic pH (endosomal) and neutral or slightly anionic at neutral physiologic pH were preferable with regard to activity and selectivity.

Optimization of the PEG-lipid component is critical for the delivery and potency of targeted LNPs. While short-chained anchored PEG-lipids, like DMG-PEG, are typically employed for delivery to the liver following intravenous (IV) administration or to lymph nodes in vaccine development employing IM administration, engaging immune cells in the blood and peripheral tissues was hypothesized to require greater stabilization and longer circulation lifetimes. Thus, we compared PEG-lipid conjugates with varying anchor lengths, di-C14 (DMG-PEG), di-C16 (DPG-PEG), and di-C18 (DSG-PEG), and backbone chemistries (diacylglycerol versus dialkylphosphatidylethanolamine) (Structures are shown in FIGS. 83B and 83C. To optimize the PEG component of the formulation we compared CD3 Fab-targeted LNP formulations based on the ionizable lipid KC2 containing 2.5% DMG-PEG, DPG-PEG, and DSG-PEG, respectively, in vivo.

Here, we observed that DPG-PEG yielded the highest transfection efficiency in T-cells (FIG. 76F and FIG. 84I-FIG. 84L). Additionally, we tested 1.5% and 2.5% DMG-PEG and DPG-PEG in vivo where we observed 1.5% to be superior to 2.5% for both DMG-PEG and DPG-PEG, and with 1.5% DPG-PEG showing the highest transfection efficiency of the tested formulations for T-cell transfection in vivo.

### An Anti-CD8 VHH Facilitates Specific Uptake in Human T-Cells

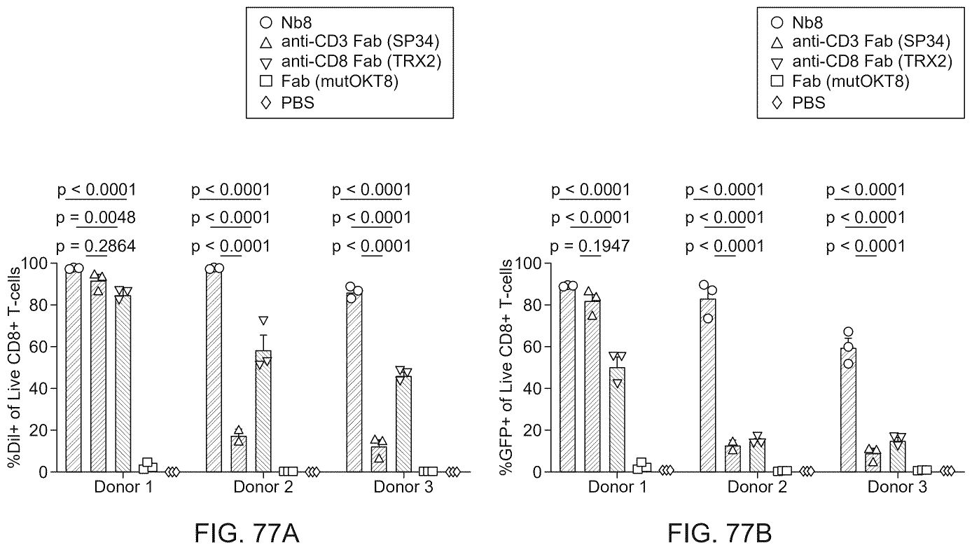

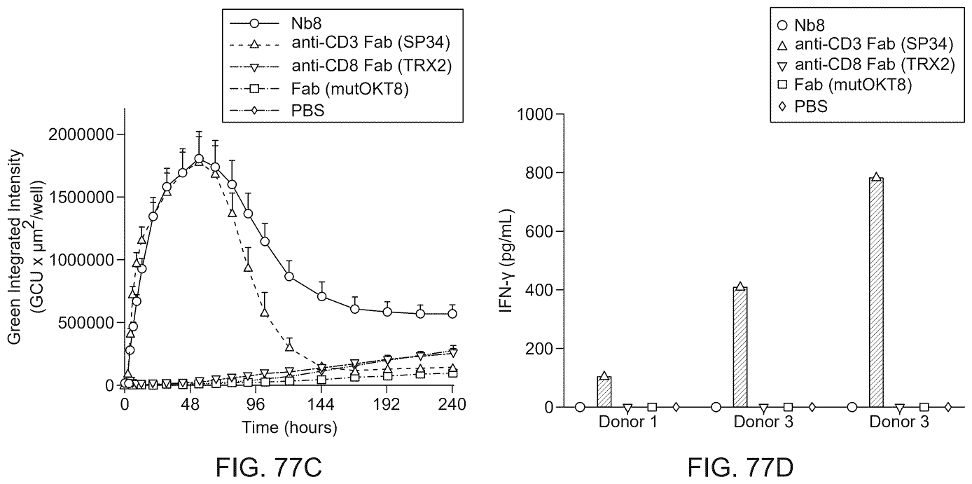

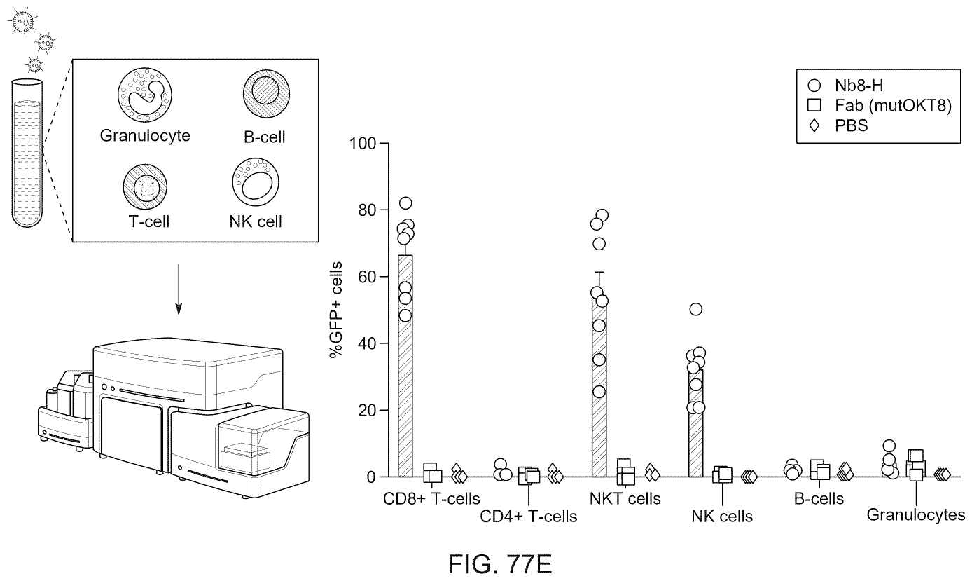

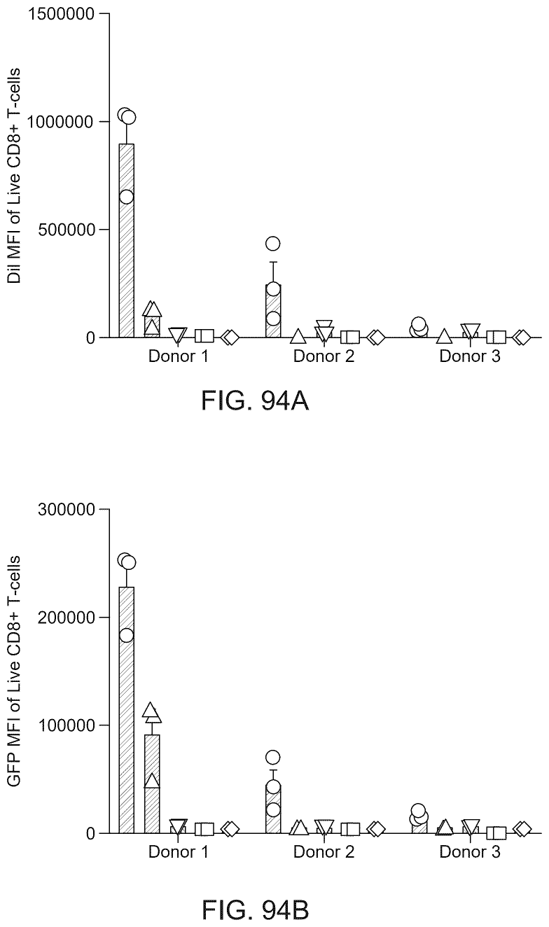

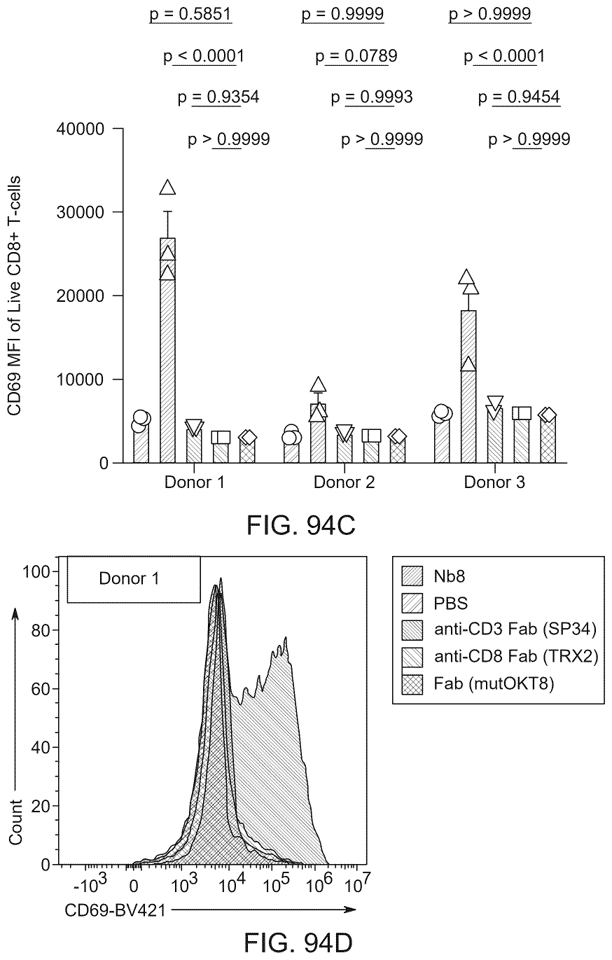

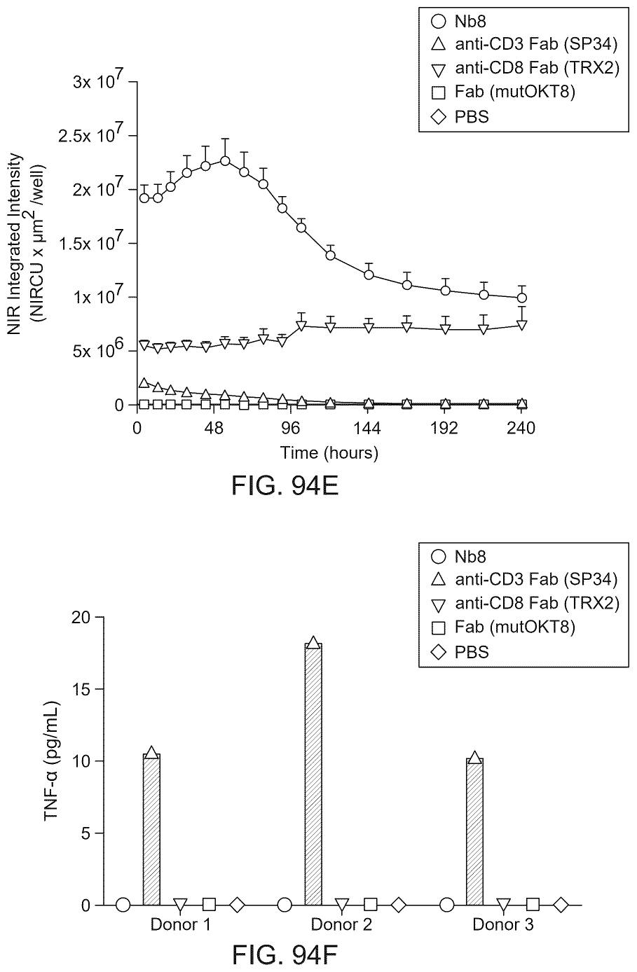

Next, using the selected Lipid 15, we evaluated the transfection efficiency of LNPs inserted with various targeting moieties against different T-cell targets. Here, we tested Fabs against CD3 and CD8, respectively, as well as a VHH against CD8 (Nb8) and found that increased transfection efficiency at various doses and time points could be achieved using the CD8-targeting VHH (FIG. 77A-FIG. 77C and FIG. 94A, FIG. 94B, and FIG. 94E). Additionally, CD8-mediated transfection did not result in elevated release of IFN-γ (FIG. 77D) and TNF-α (FIG. 94F), nor upregulation of the early T-cell activation marker, CD69 (FIGS. 94C and 94D), as opposed to transfection using CD3-targeted LNPs.

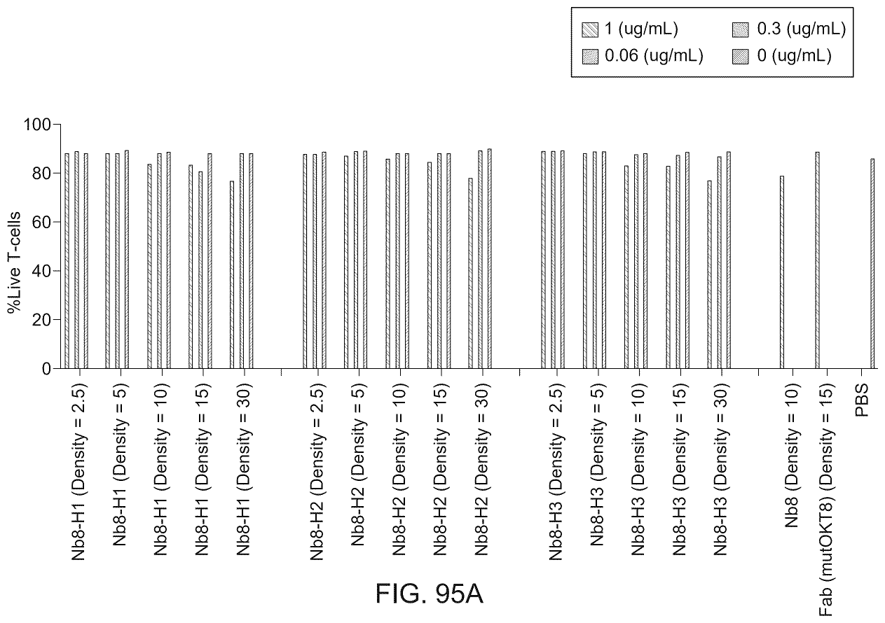

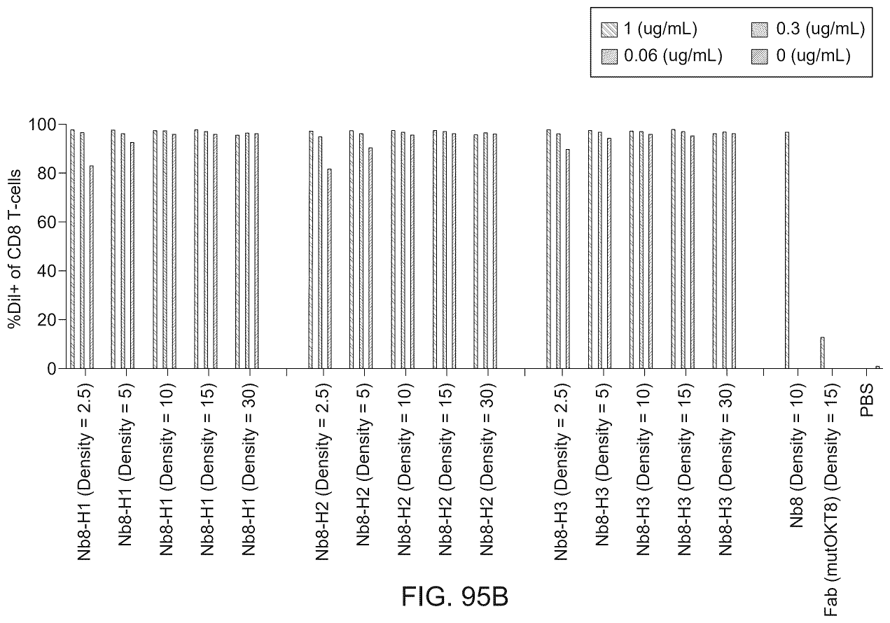

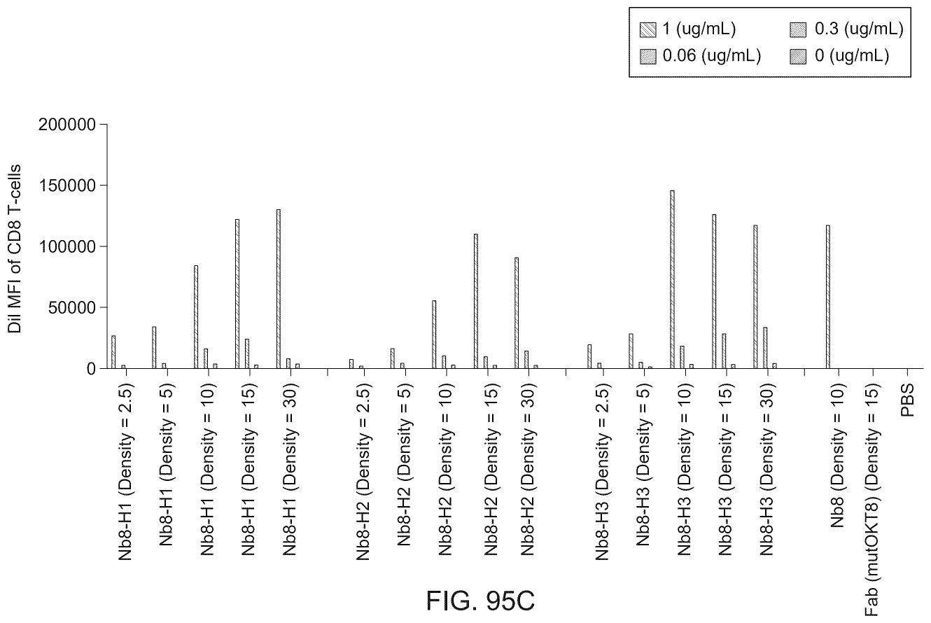

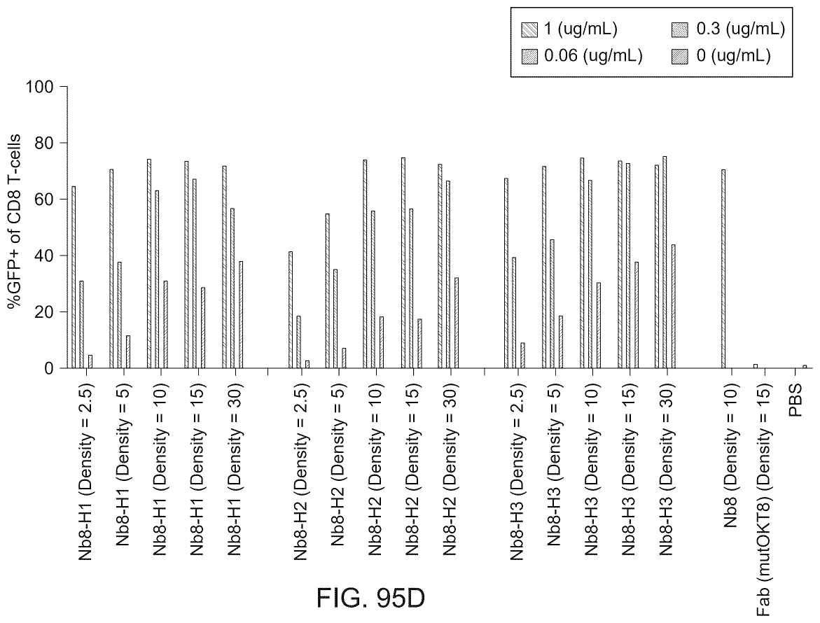

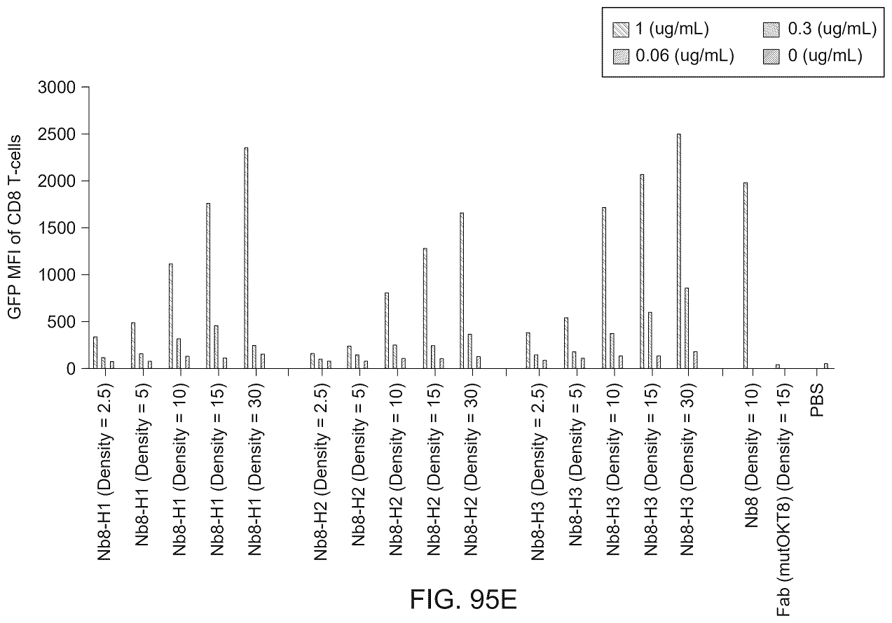

Following the selection of Nb8 as the lead targeting moiety, the amino acid sequence was further optimized to make the binder more human-like, to reduce its binding by pre-existing antibodies, and to improve its long-term stability. The resulting optimized variants were conjugated, inserted into LNPs, and compared by transfecting T-cells in vitro. Here, Nb8-H3 showed superior binding and GFP expression compared to other variants tested and was thus selected as the targeting moiety for subsequent experiments (FIG. 95A-E).

Having observed high transfection efficiency in isolated T-cells, we next sought to investigate the level of delivery specificity. To do this, we developed an ex vivo assay, testing human whole blood from 8 healthy donors to evaluate the expression efficiency in various cell subsets by Flow Cytometry (FIG. 77E, left panel). Here, we did not observe significant off-target cell transfection in the Granulocyte and B-cell populations (FIG. 77E, right panel). Additionally, and as expected, the CD8-targeted LNPs showed high transfection efficiency in the CD8+ cell subsets, including CD8⁺ T-cells, NKT cells, and NK cells, while no significant GFP expression was observed in the CD4⁺ T-cell population. Generally, we observed modest differences in transfection efficiencies between the donors tested, but all 8 donors showed a high degree of cell specificity as no GFP expression could be observed in off-target cell populations.

### a Novel CD22 CAR Mediates Target Cell Killing In Vitro

To demonstrate the translatability of the platform and to investigate whether the targeted LNPs could be used to reprogram T-cells into CAR T-cells, we first designed a CD22 CAR mRNA library containing various CD22 binders incorporated into second-generation CAR cassettes with different hinge domains and lengths, a CD28 co-stimulatory domain, and a CD32 signaling domain (FIG. 96A, left panel). The CD22 CAR mRNAs were tested in a Jurkat activation assay where we found 22-V4 containing a CD28-based hinge to show the highest activity, assessed by the upregulation of CD69 after 24 hours of co-culturing with Raji cells (FIG. 96A, right panel). Additionally, mRNAs encoding 22-V4 containing a CD28 co-stimulatory domain and 22-V4 containing a 4-1BB co-stimulatory domain, respectively, were formulated into targeted LNPs and tested in a cytotoxicity assay against Nalm6 cells. Here, the 22-V4 construct containing a CD28 co-stimulatory domain showed slightly improved cytotoxicity over the construct containing a 4-1BB domain (FIG. 96B). Thus, the 22-V4 binder containing a CD28 hinge, a CD28 co-stimulatory domain, and a CD3ζ signaling domain was selected as the lead CD22 CAR.

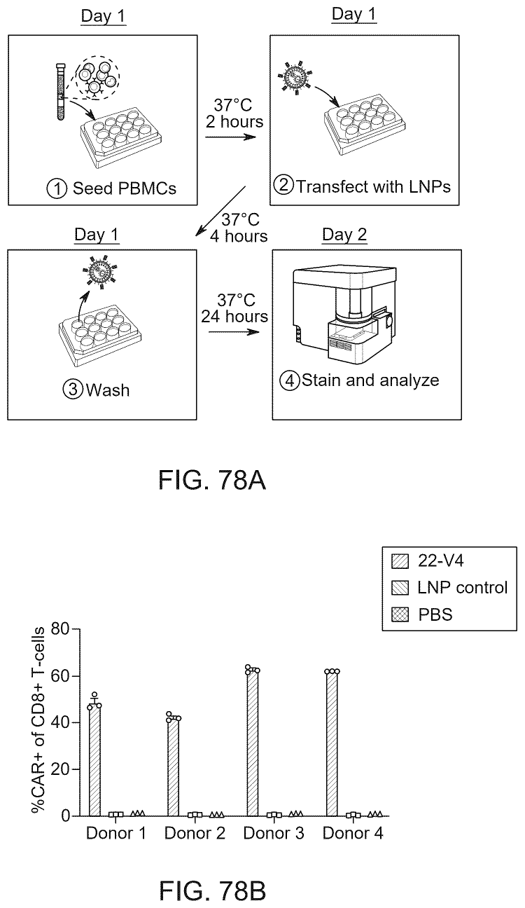

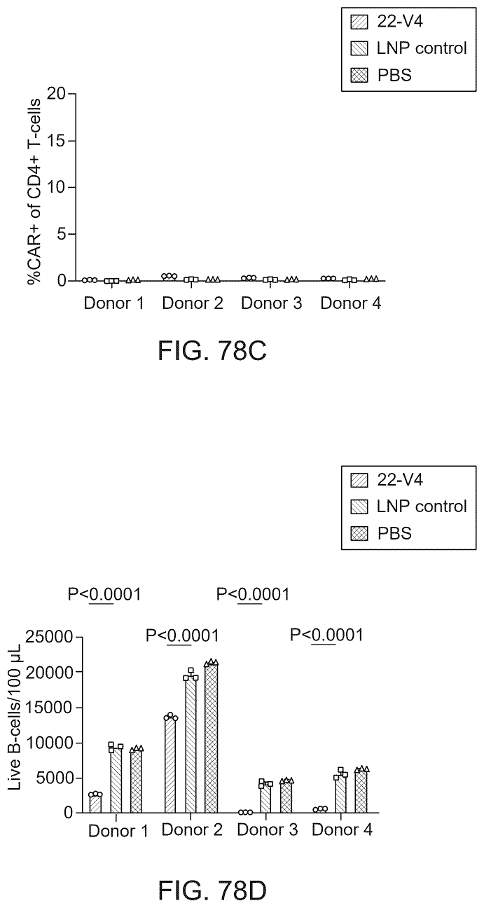

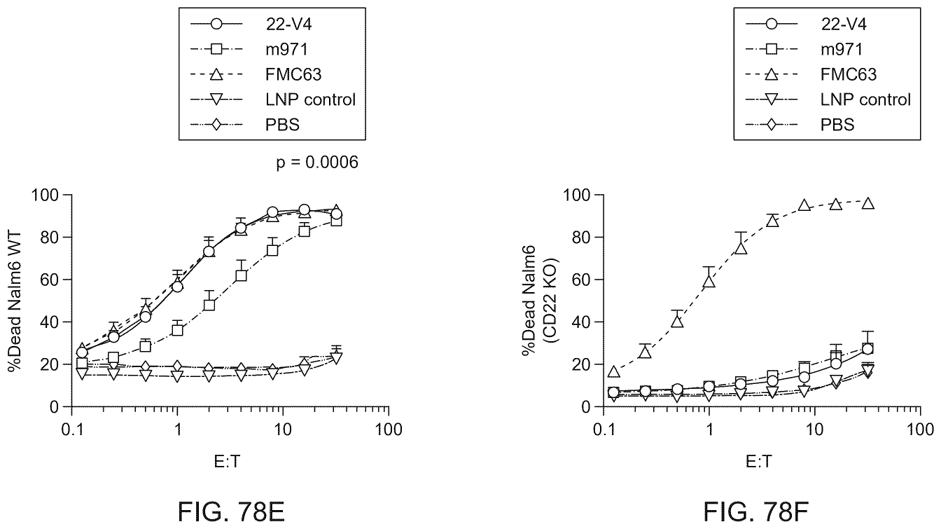

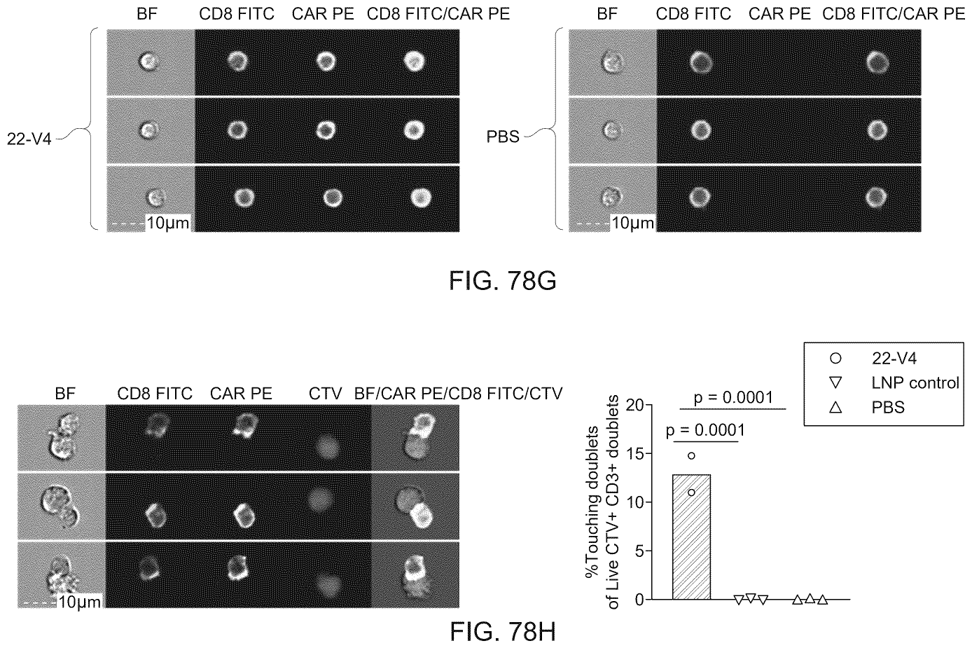

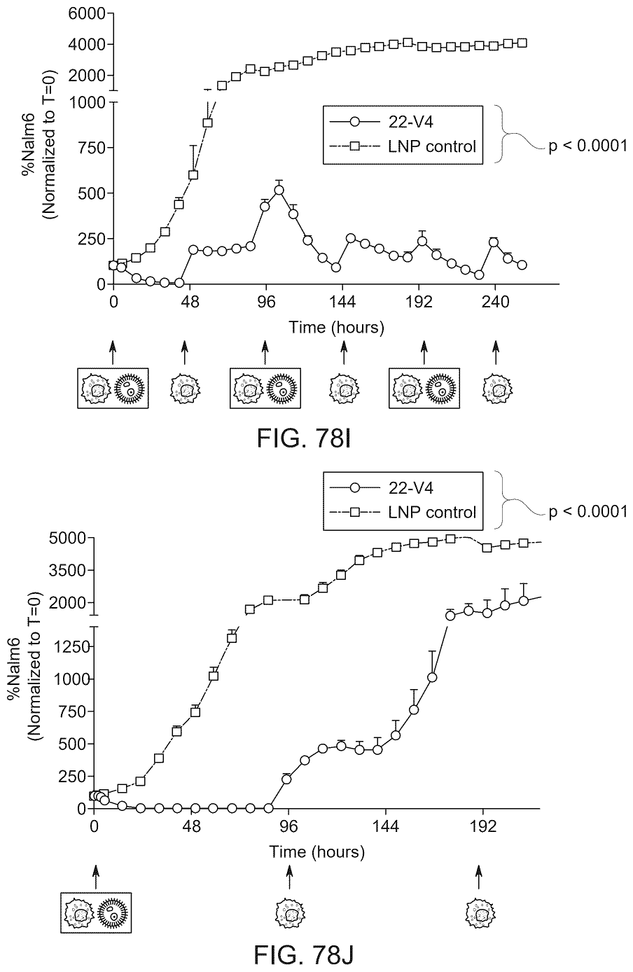

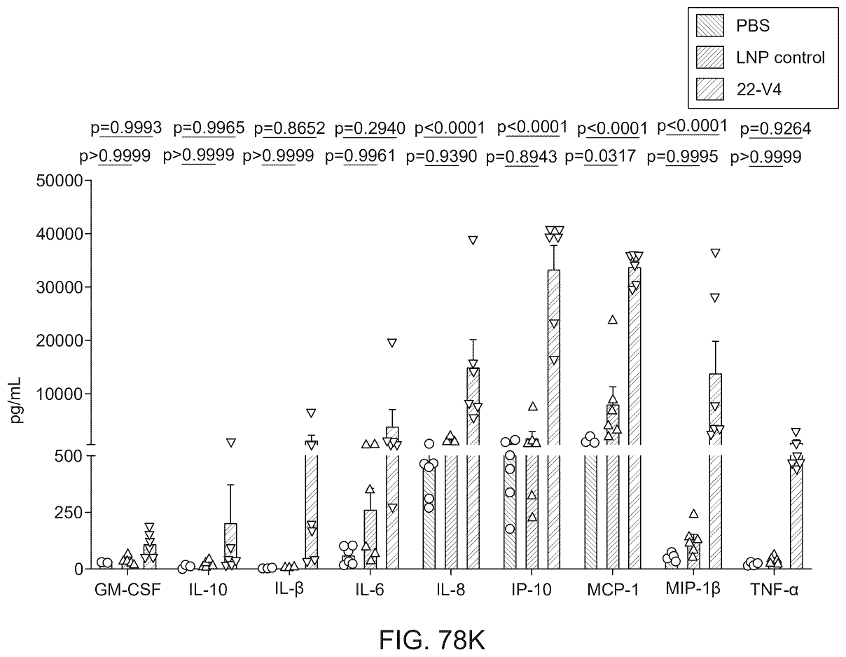

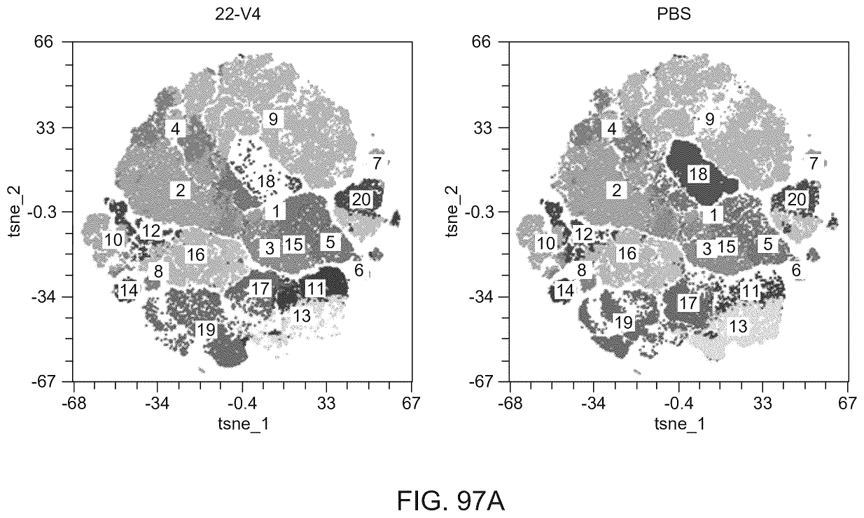

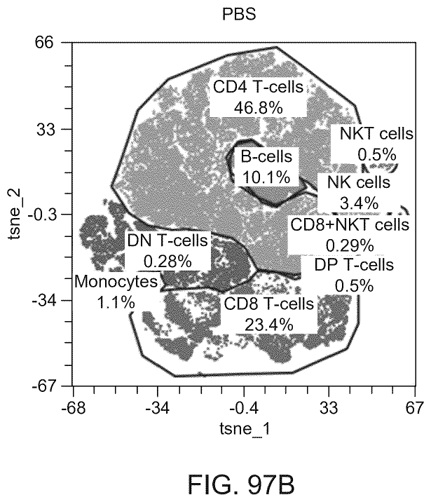

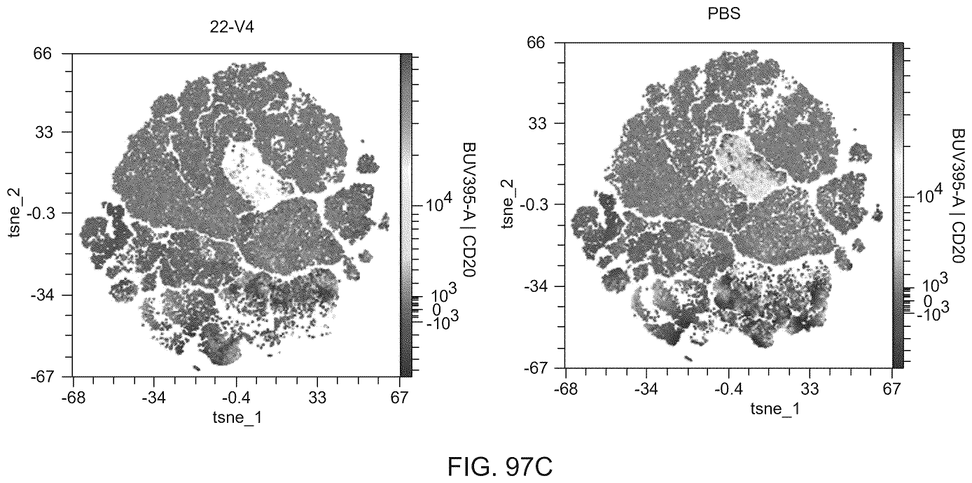

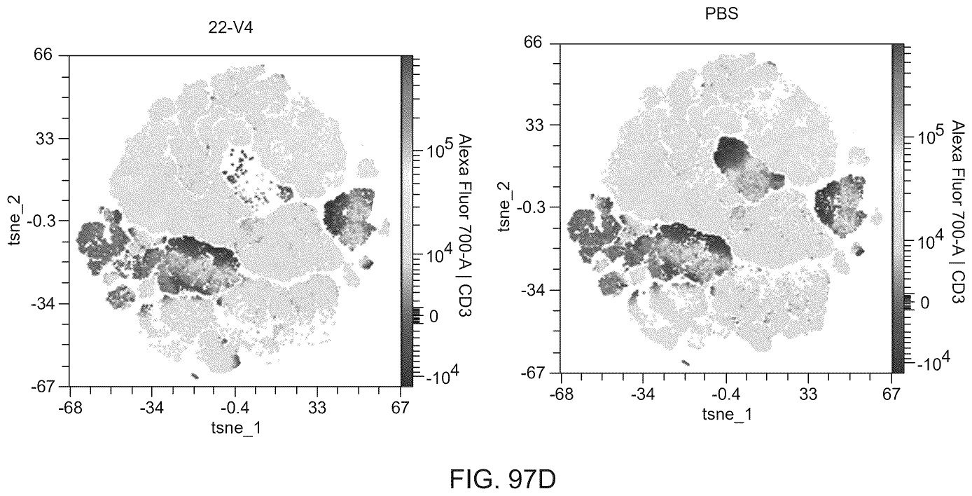

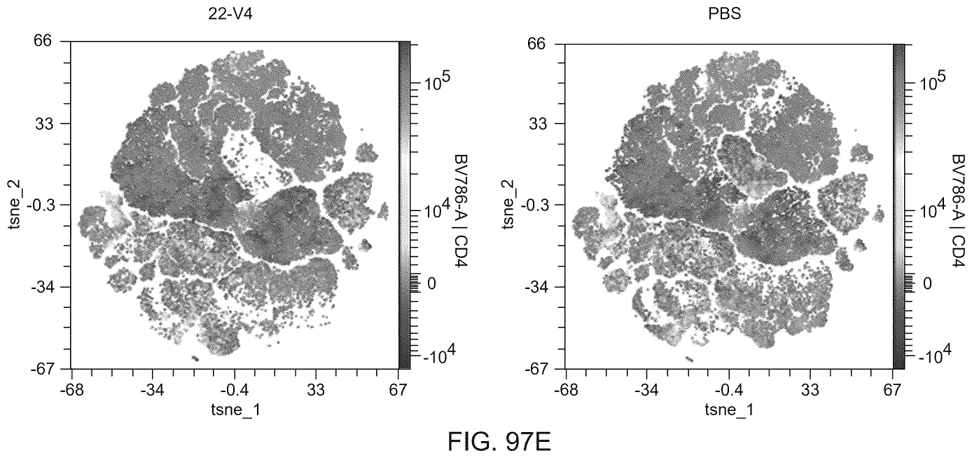

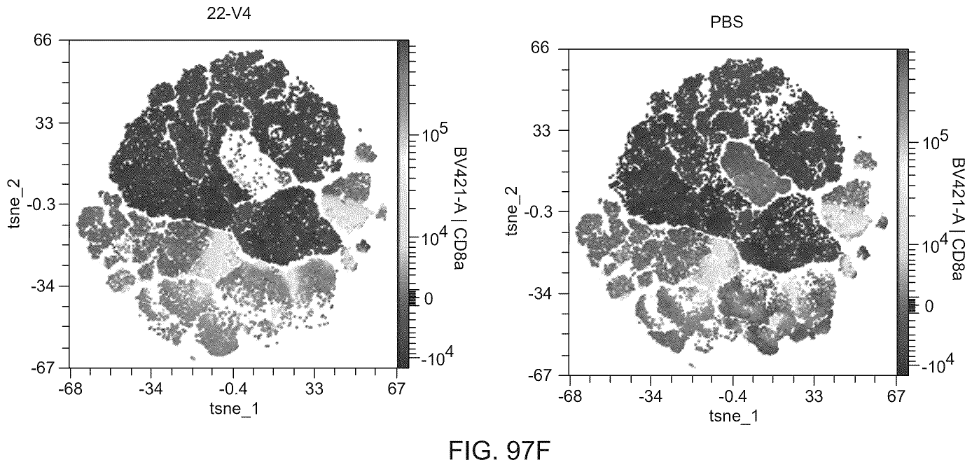

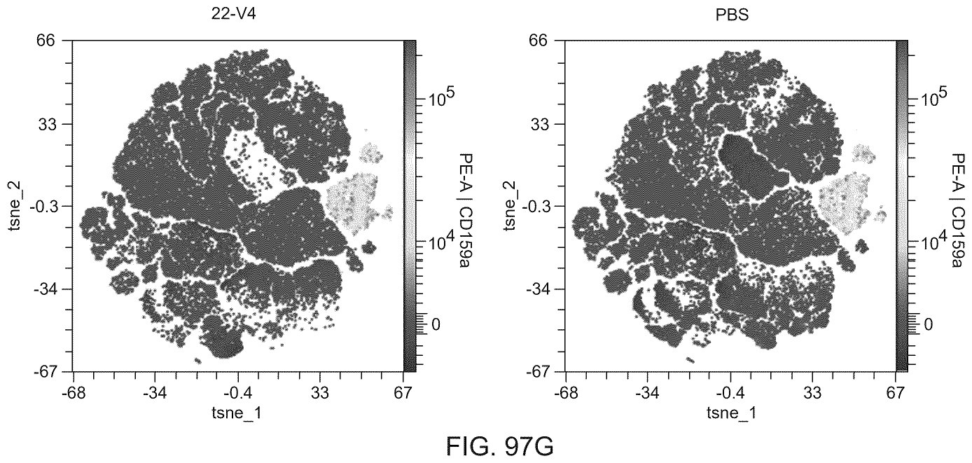

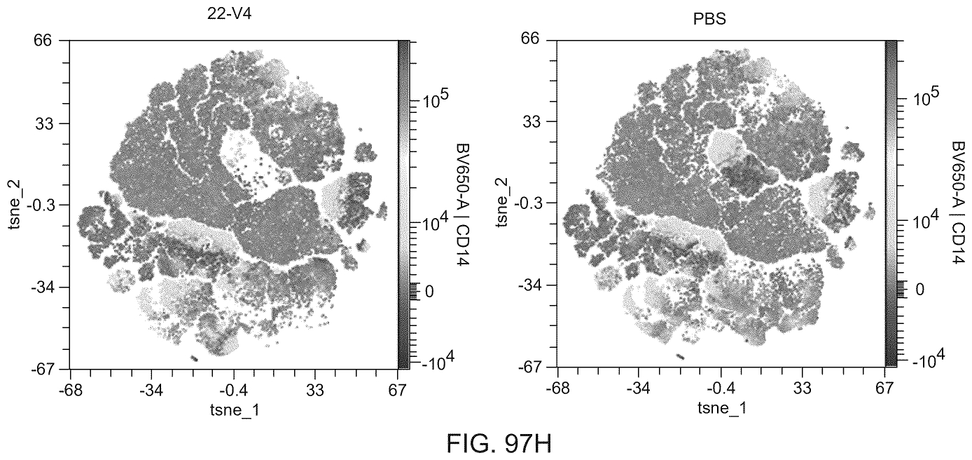

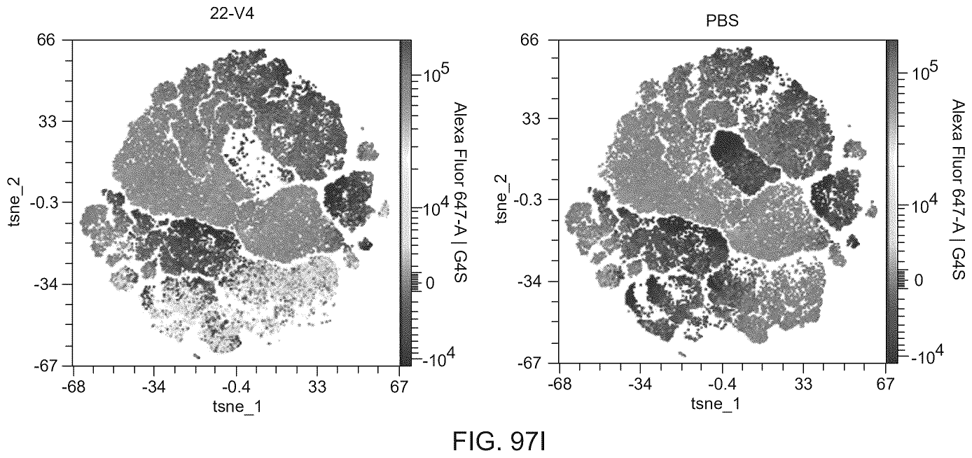

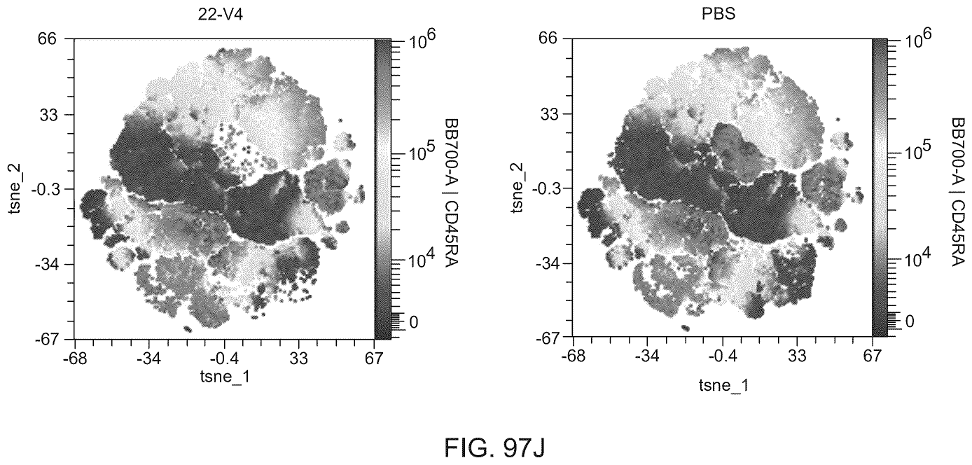

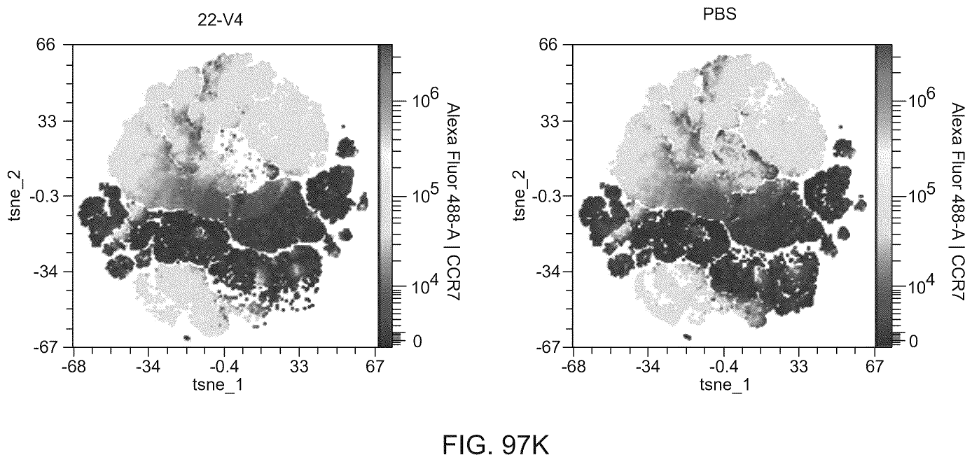

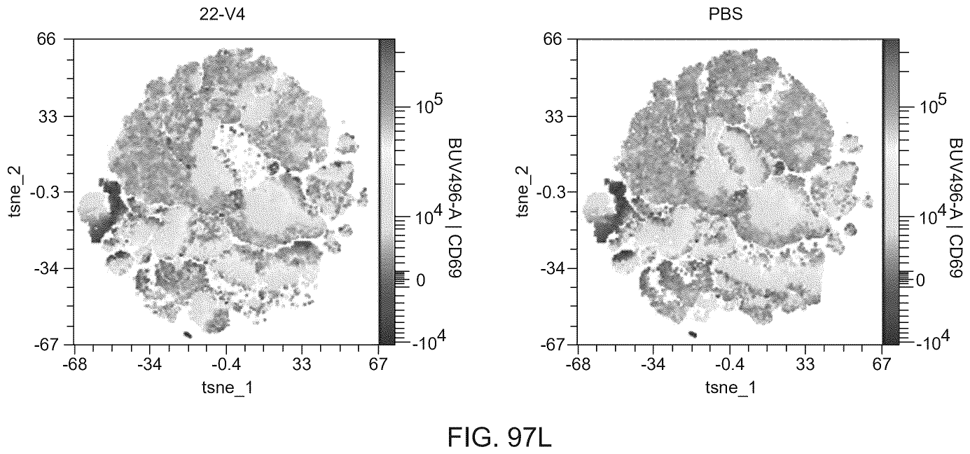

To test the transfection specificity and B-cell aplasia of 22-V4 in vitro, we transfected PBMCs from 4 healthy human donors and evaluated the CAR expression and B-cell counts, 24 hours after LNP addition (FIG. 78A). Here, we observed robust CAR expression in the CD8+ T-cells in all donors tested (FIG. 78B) while no CAR expression was observed in the CD4+ T-cell population (FIG. 78C) or other off-target populations (FIG. 97A-L). B-cell counts were significantly decreased in the 22-V4-LNP treated group for all donors compared to the non-CAR LNP (Fluc) treated group demonstrating CAR-mediated B-cell aplasia (FIG. 78D).

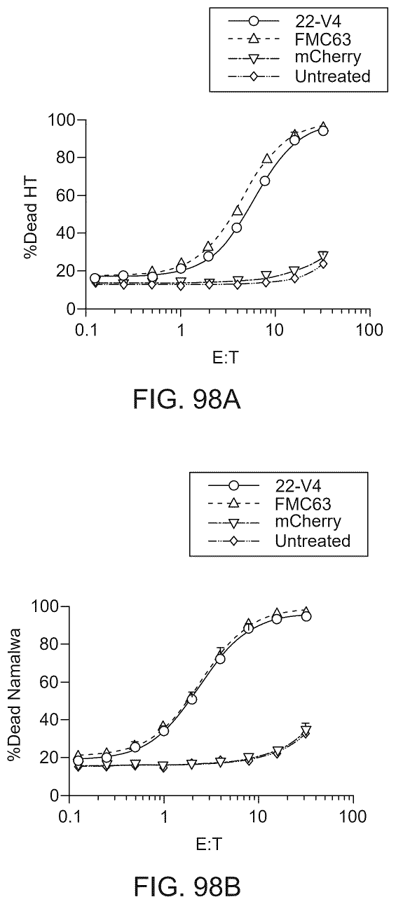

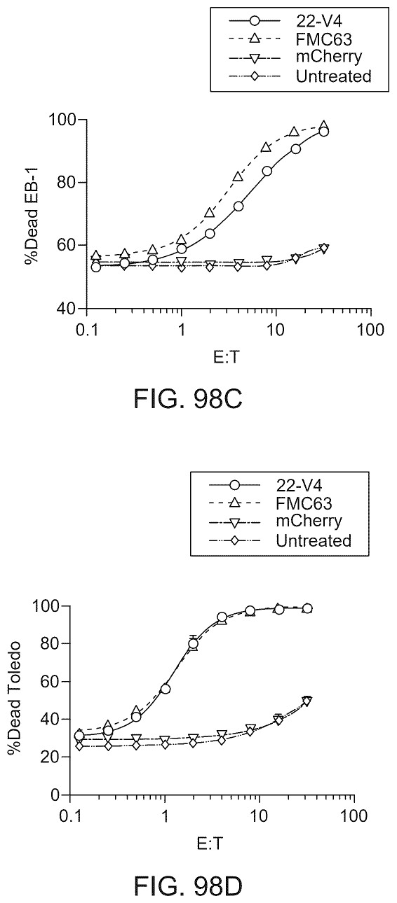

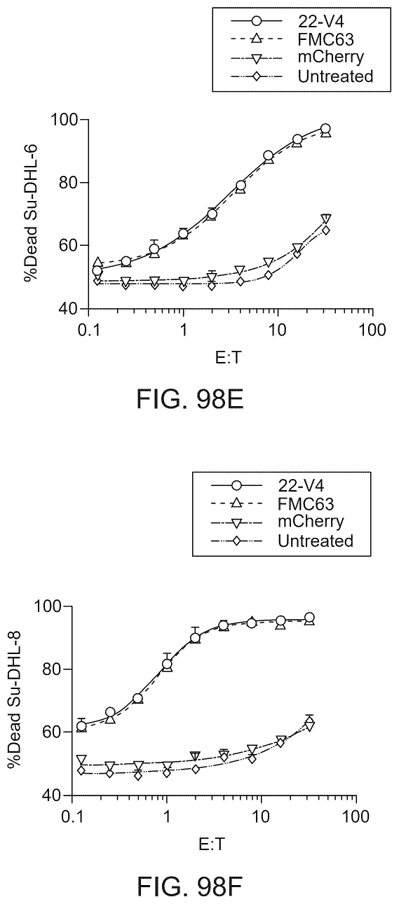

To further compare and validate the novel 22-V4 construct, two clinically relevant CAR constructs against CD19 (FMC63, [39]) and CD22 (m971, [40]), respectively, were, similar to 22-V4, encoded into mRNAs in a second-generation CAR format and encapsulated into CD8-targeting LNPs. The CAR constructs were first verified to be expressed on the surface of primary human T-cells by staining for the CD19 CAR and CD22 CARs, respectively (FIG. 16E-H). Following LNP transfection, CAR T-cells were co-cultured with relevant human cancer cell lines expressing different levels of target antigens, CD19 and CD22 (FIG. 16C and FIG. 16D). 22-V4-expressing T-cells showed improved CAR-mediated cytotoxicity against Nalm6, JVM-2, and Reh cells over T-cells expressing the clinical m971-based CAR construct (FIG. 78E and FIG. 96M and FIG. 96N, respectively). Additionally, 22-V4-mediated killing was shown to be target-specific as no cytotoxicity was observed against the Nalm6 and Raji CD22 knockout (KO) cell lines and the K562 CD22-negative cell line (FIG. 78F and FIG. 96J and FIG. 96L). However, no difference in CAR-mediated cytotoxicity between the constructs was observed against Raji and Daudi cells (FIGS. 961 and 96K). Additional relevant target cell lines were tested for CAR-mediated cytotoxicity (FIG. 98 and Supplemental Table 9).

To visualize the 22-V4 CAR expression we utilized the Amnis® Imagestream where a surface-based CAR-stain could be observed in CD8+ T-cells treated with LNPs encapsulating 22-V4-encoding mRNA in comparison to non-treated CD8+ T-cells (FIG. 78G). Additionally, the 22-V4 CAR-expressing CD8+ T-cells were co-cultured with CTV-stained Nalm6 cells. Here, we identified the CAR-forming synapsis between the 22-V4 CAR-expressing T-cell and the CD22-expressing target cell emphasizing that CAR-mediated engagement results in target cell killing (FIG. 78H).

Next, we quantified the cytokines secreted into the supernatants by T-cells expressing the different CAR constructs after co-culture with Raji cells. Expectedly, we found that the cytokine concentrations correlated with the level of target cell killing decreasing with the effector-to-target ratios (E:Ts) and with no cytokines being detected in samples containing T-cells not receiving antigen stimulation (E:T=4:0) suggesting the absence of tonic signaling for all CAR constructs tested (FIG. 96O-4Q). To investigate the killing kinetics of T-cells transfected with the targeted CAR mRNA-LNPs, we designed a repeated killing assay where target cells were added to transfected T-cells every two days with LNP re-addition every four days or where target cells were added to transfected T-cells every four days without LNP re-addition. Interestingly, we observed that target cell killing can be achieved continuously for at least 10 days with re-transfection (FIG. 78I) and for up to around 8 days after a single LNP transfection (FIG. 78J).

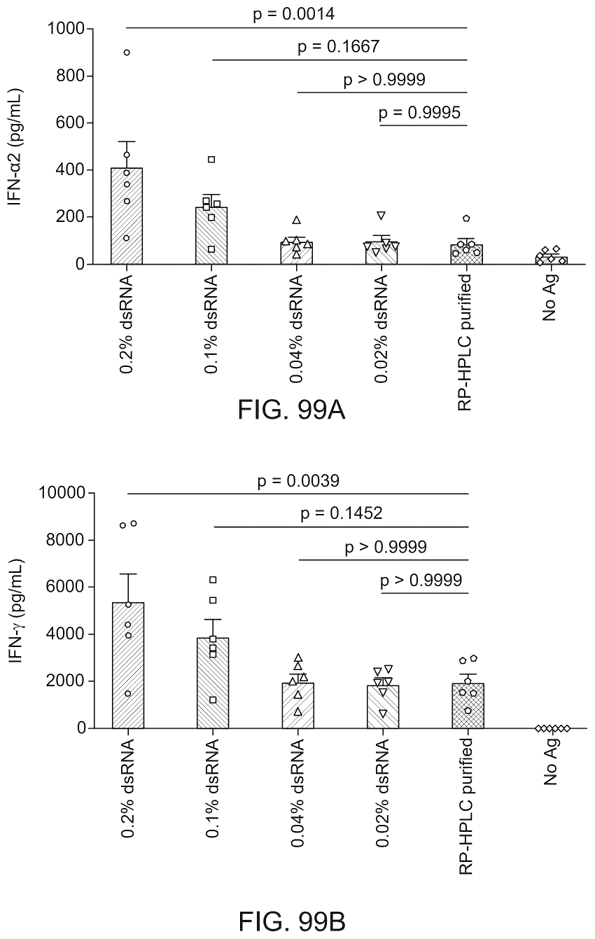

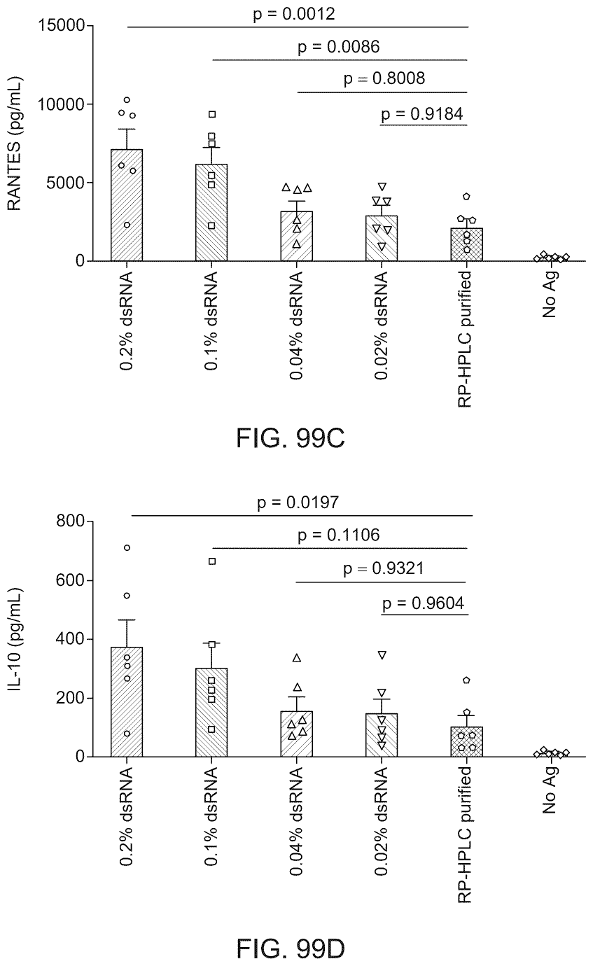

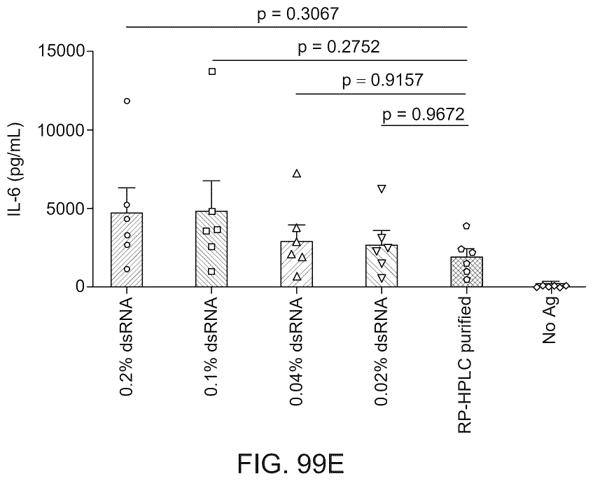

To get a better understanding of how the human immune system would respond to the various components of our CD8-targeted LNP we utilized the Modular IMmune In-vitro Construct (MIMIC®) technology [36], enabling imitation of the human immune system in a well-based format. We first sought to compare cells treated with targeted LNPs containing a non-CAR encoding mRNA payload (mCherry) to CD22 CAR, 22-V4, encoding mRNA to investigate the effect of uptake of the targeted LNP versus the subsequent expression and target engagement of the CD22 CAR. Generally, we observed an increase in cytokine release when cells were treated with LNPs containing the CAR payload indicating the expected effect of CAR-mediated target engagement and activation (FIG. 78K). However, for non-CAR LNP treated samples, a significant increase in the release of some cytokines, including IL-6, IL-8, MIP-1β, and TNF-α, over untreated samples were observed suggesting that the targeted LNP-mediated cell uptake plays a role in stimulating parts of the immune system. Next, we evaluated the cellular response to varying amounts of double-stranded RNA (dsRNA) resulting as a byproduct from the IVT process, as this is known to be immunogenic and may trigger an innate immune response [41]. Here, we observed that increased levels of cytokines were secreted when cells were treated with targeted LNPs formulated with mRNA containing up to 0.2% dsRNA in comparison to RP-HPLC-purified mRNA (FIG. 78L and FIG. 99A-FIG. 99E). These data suggests that initial purification of the mRNA prior to formulation is critical to reduce the potential immunogenicity of the targeted LNPs.

### CD8-Targeted LNPs Specifically Reprogram Functional CAR-T Cells In Vivo

Following promising data in vitro we next investigated the delivery efficiency to T-cells in vivo. Here, NSG mice were engrafted with human PBMCs and IV injected with a clinically relevant dose of targeted LNPs (0.3 mg/kg). 24 hours after treatment, >20% CD22 CAR expression could be detected on CD8⁺ T-cells in the blood, spleen, bone marrow, and lungs (FIG. 79A). Next, we evaluated the CD22 CAR expression in CD8⁺ T-cells after repeated dosing performed at a similar dose level. After a fourth dose, we observed a level of CAR expression in the CD8⁺ T-cells in the blood that was similar to the level of CAR expression observed after the first dose (FIG. 79B). Thus, this suggests the feasibility of repeatedly administering the targeted LNP. Additionally, as a surrogate for efficacy, we evaluated the percent of human CD19+ cells (B-cells) in the spleen and bone marrow. Here, we observed a decrease in % B-cells in the CAR-LNP (22-V4)-treated group in both organs compared to vehicle-treated animals (FIG. 79C). From these studies, we evaluated the body weight (BW) during dosing and spleen weight after the final fourth dose as indicators of toxicity. From these evaluations, no significant differences between the two groups could be observed (FIG. 79D and FIG. 79E).

To assess the level of transfection specificity in vivo we stained for various cell subsets in the liver after IV injecting CD8-targeted Lipid 15 LNPs encapsulating GFP-encoding mRNA. Here, only CD8+ T-cells showed increased GFP expression over the baseline demonstrating the level of LNP specificity (FIG. 79F). The expression levels in endothelial cells, macrophages, and hepatocytes were less than 5% in all populations, indicating the efficacy of the de-targeting strategy employed in the design of the LNP.

To summarize, beyond efficacy and as demonstrated in the previous sections, each component of the targeted LNP was thoroughly designed and tested to be highly tolerable. The targeting moiety was designed to decrease transfection-mediated activation and thus cytokine release. The chemical structure of the ionizable lipid and the lipid composition were developed to de-target the liver and off-target cell populations. The CAR was engineered to circumvent tonic signaling and cytokine secretion in the absence of target engagement. Purification methods were deployed to limit the presence of mRNA impurities, including double-stranded mRNA.

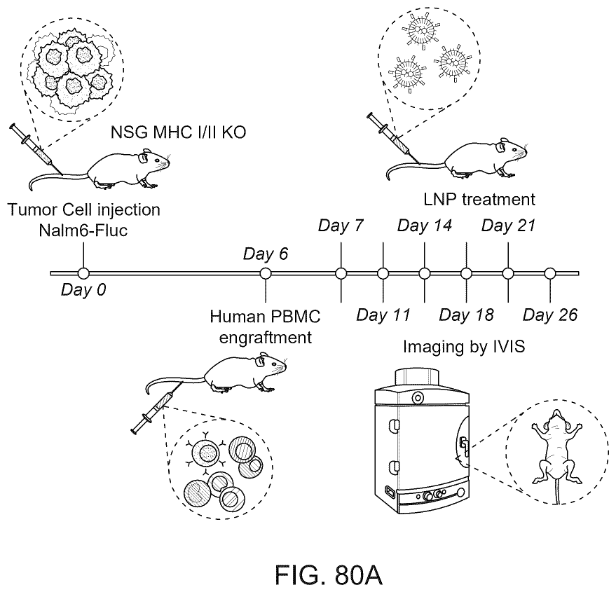

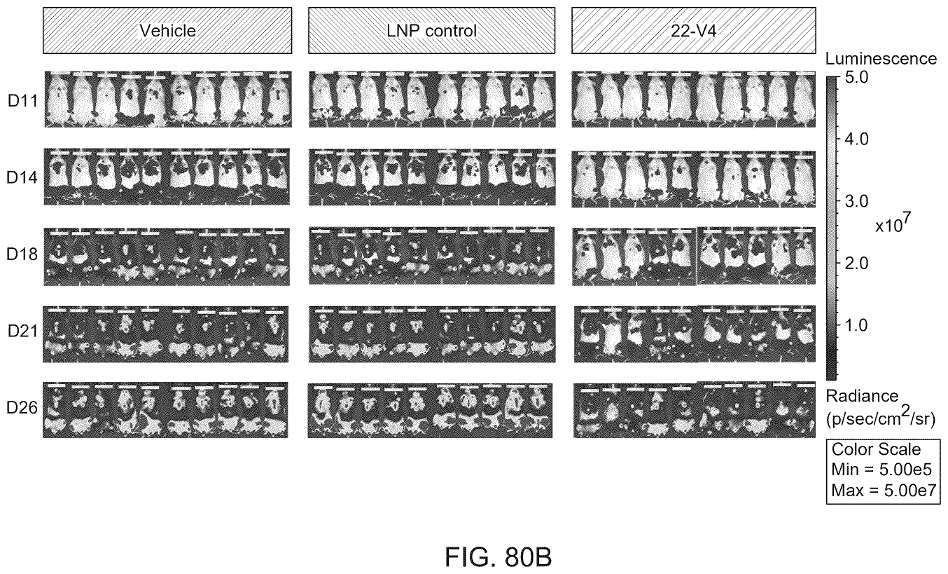

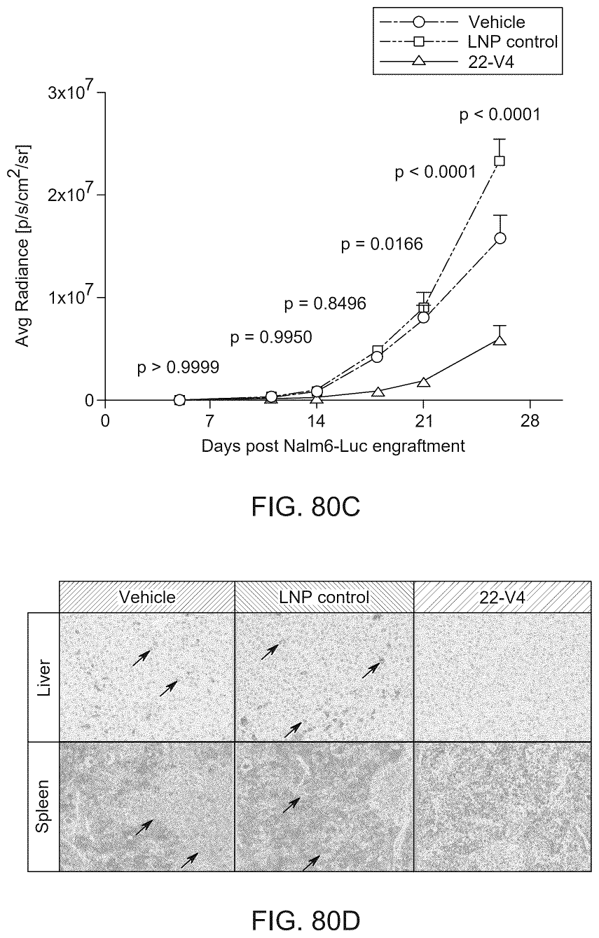

Finally, to evaluate the efficacy of the CAR-LNP in vivo, we established an aggressive humanized Nalm6 tumor model (FIG. 80A). Throughout repeated CAR-LNP dosing, tumor control was maintained for the duration of the study as measured by IVIS (FIG. 80B and FIG. 80C). Additionally, immunohistochemistry (IHC) was performed on the spleen and liver on day 26 to evaluate the level of organ-specific efficacy. Here, we observed that CD22-expressing tumor cells were eliminated from the spleen and liver (FIG. 80D and Supplemental Tables 10 and 11).

### Discussion

Throughout the last decade, clinical results with CD19 CAR T-cells have demonstrated the potential that CAR treatments hold as an immunotherapy against cancer [42, 43]. Chimeric antigen receptors (CARs) have proven successful in treating hematological malignancies with the FDA approval of Kymriah and Yescarta in 2017. However, the leukapheresis process and ex vivo transduction and expansion to generate autologous CAR-T cells before infusing them back into the patient are cumbersome and costly. However, current CAR-T cell therapies for B-cell malignancies require elaborate and expensive processes to manufacture engineered T-cells ex vivo, which provides a barrier to making them standard-of-care treatments and available to the broad patient population [44]. One solution which we explore here preclinically is to reprogram the patient's T-cells to become CAR-T cells directly in vivo thereby overcoming the challenges associated with patient T-cell isolation, genetic modification, and selective expansion ex vivo. Here, we explored an mRNA delivery strategy using targeted lipid nanoparticles (LNPs) to transfect and reprogram human T-cells in vivo. We demonstrate that in vitro transcribed (IVT) mRNA encoding three different B-cell targeting chimeric antigen receptors (CARs) can be delivered specifically to reprogram T-cell subsets in vitro and in vivo.

To accomplish this, messenger RNA (mRNA) can be utilized as it provides advantages over DNA-based approaches. mRNA is naturally translated to the encoded protein in the cytoplasm, and it thus circumvents the need for the payload to enter the nucleus of the cell to achieve protein expression, which is recognized as being challenging to accomplish. Importantly, this also eliminates the risk of integration into the genome providing a distinct safety benefit. However, to be efficiently and safely delivered to target cells in vivo, mRNA needs to be encapsulated into a vector to protect the nucleic acid from ribonuclease-mediated degradation, enable uptake across the cell membrane, and reduce unwanted activation of the innate immune system.

Viral-based vectors have been explored extensively for delivering nucleic acids in vivo. However, a critical downside of delivery systems derived from viruses is the risk of immunogenicity and the potential for integration into the genome [45, 46]. Additionally, the recent black box warning from the FDA on the virus-generated CAR-T products related to the life threatening CRS and neurologic toxicities, as well as secondary resulting T-cell malignancies, has emphasized the need for new non-viral approaches.

An extensive number of non-viral in vivo delivery technologies have been explored with the most prevalent ones being lipid and polymer-based nanoparticle systems. Here, polymer-based systems have in some cases shown to present a greater risk of inducing an immunogenic response, as well as possess difficulties with formulating storage-stable nanoparticles in comparison to liposomes or lipid nanoparticles [47, 48]. Rationale engineering is possible to reduce nonspecific cell interactions while efficiently targeting uptake and transfection of target cells, minimizing toxicity, and providing for circulation lifetimes that allow for optimal engagement of the targeted T-cells.

We chose to encapsulate anti-CD19 and -CD22 CAR-encoding mRNA payloads in our targeted LNPs based on the clinical success of CAR-T cell therapies against B-cell malignancies [49-51]. With this, we show that T-cells can be reprogrammed to transiently express three different functional CARs using targeted LNPs. With this, our data demonstrate the platform can be used to achieve specific killing of target-expressing cancer cell lines down to low effector-to-target cell ratios in vitro while also achieving efficacy in a relevant human in vivo cancer model.

As a proof-of-concept, we show that targeting the highly expressed T-cell receptors, CD3 and CD8 are efficient for reprogramming T-cells and for driving target cancer cell killing. However, as this approach is highly adaptable, our group is also interested in using the developed platform to explore the targeting of other immune cell types and surface markers to deliver therapeutically relevant mRNA, which has been shown by others to be useful in developing new disease treating strategies [24-26].

In vivo reprogramming of specific cell types such as T lymphocytes is an emerging and rapidly growing field as indicated by several recent published studies [23, 26, 28, 29, 52]. However, to our knowledge, this is the first report demonstrating a VHH-targeted LNP system to specifically deliver CAR-encoding mRNA to CD8+ T-cells directly in vivo to treat hematological malignancies.

In conclusion, our work demonstrates an efficient and highly adaptable LNP-based platform that can be utilized to specifically deliver relevant mRNA therapeutics to T-cells. The report shows that the approach can be used to reprogram T-cells to become functional cancer-killing CAR-T cells demonstrating one of many applications for which this strategy can be utilized. In general, this platform holds promise for becoming a first-in-class “off-the-shelf” CAR-T cell therapy, overcoming significant economic and biological barriers associated with current approved ex vivo CAR-T therapies.

### Supplemental Information

### LNP Characterization

The LNPs were characterized to determine the average hydrodynamic diameter, zeta potential, and mRNA content (total and dye-accessible). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles was measured using Thermo Fisher Quant-iT RiboGreen RNA Assay Kit. Dye-accessible RNA, which includes both non-incorporated RNA and RNA that is near the surface of the nanoparticle, was measured by diluting the nanoparticles to approximately 1 μg/mL mRNA using HEPES buffered saline, and then adding Quant-iT reagent to the mixture. Total RNA content was measured by diluting the particles to 1 μg/mL mRNA using HEPES buffered saline, disrupting the nanoparticles by heating them to 60° C. for 30 minutes in HEPES buffered saline containing 0.5% Triton, and then adding Quant-It reagent. RNA was quantified by measuring fluorescence at 485/535 nm, and concentration was determined relative to a contemporaneously run RNA standard curve.

### Cryogenic Electron Microscopy (Cryo-EM)

Cryo Transmission Electron Microscopy was performed by NanoImaging Services (San Diego, CA). Samples were prepared by applying a 3 μL drop of sample suspension to a cleaned grid, blotting with filter paper, and then vitrifying the sample in liquid ethane. Electron microscopy was performed using a Thermo Fisher Scientific Glacios Cryo Transmission Electron Microscope (Cryo-TEM) operated at 200 kV and equipped with a Falcon 4 direct electron detector. Images were acquired at a nominal underfocus of −4.1 μm to −2.0 μm and electron doses of ~11-21 e⁻/Å².

Novel Ionizable Cationic Lipid Development for T-Cell Reprogramming with Targeted LNPs

A novel ionizable lipid family (of the general structure shown in FIG. 83A) was screened in targeted LNP formulations to identify the optimal molecular features for reprogramming CD8-positive T-cells. Lipid selection criteria included the physiochemical properties of the resulting LNPs as well as the ability of the resulting formulations to illicit reporter protein expression in an in vitro primary human CD8⁺ T-cell transfection experiment using αCD8− targeted LNPs.

Molecular design features of this novel lipid family included the use of an aminopropyl diol linker group to bridge the dimethyl aminopropyl ionizable head group to two hydrophobic lipid “tail” groups via degradable ester linkages and a single less hydrophobic N-acyl amide linked “side” group. The two “tail” groups and the single “side” group were chosen to impart an asymmetric structure to lipid molecule, and the resulting cone-shaped structure was additionally amplified using either degree of unsaturation or by incorporation of branched aliphatic substituents. Furthermore, degradable ester linkages were introduced systematically into both “side” and “tail” groups to impart in vivo biodegradability and enable rapid lipid elimination. As seen in Scheme S1, in lipids 1 through 8,

N-acyl “side” group consists of 8 carbon (Lipid 5), 9 carbon (Lipids 2, 3 and 4), 10 carbon (Lipids 1 and 8), or 11 carbon (Lipids 6 and 7)O-acyl substituents in combination with either dioleoyl (lipid 4) or di-linoleoyl (Lipids 1, 2, 3, 5 & 6, 7, 8)O-acyl “tail” groups, thereby spanning a range of “side” group hydrophobic content with mono- and di-unsaturated “tail” groups. Similarly, the three-dimensional structure and steric properties of the “side” were tuned via position and degree of branching, i.e., using linear and branched (Lipid 2 versus Lipids 3 and 4), or α- and β-substitution (Lipid 6 versus 8) of the alkyl groups within the N-acyl “side” group.

In lipids 9, 10, 15, 16, 24A, and 26, a succinate-derived degradable ester linkage was introduced into the “side” group, and the hydrophobicity of the ester moiety was controlled via the choice of succinate ester composition, i.e., a less hydrophobic 3-octanol succinate (in Lipid 16) or more hydrophobic 3-decanol succinate (in Lipids 9, 10, 15, 24A and 26). Also, in this sub-set, the lipid “tail” groups comprised of di-linoleoyl (Lipids 9 and 10), di-oleoyl (Lipids 15, 16), di-(8-hydroxydecanoate) (Lipid 24A) or 4-((2-hexyldecanoyl)oxy) butanoate (Lipid 26) O-acyl substituents. These tail group compositions were chosen to evaluate the effect of the degree of lipid unsaturation (Lipids 9, 10, 15, and 16) and branching (Lipids 24A and 26) as these structural parameters are expected to modulate molecular shape (“cone” structure) and lipid melting points and thereby alter the ability of the resulting LNP formulations to enable efficient endosomal membrane disruption and cytosolic delivery of the RNA payload.

### Physiochemical Properties of Lipids 1-11 and Lipid 15 Targeted LNPs

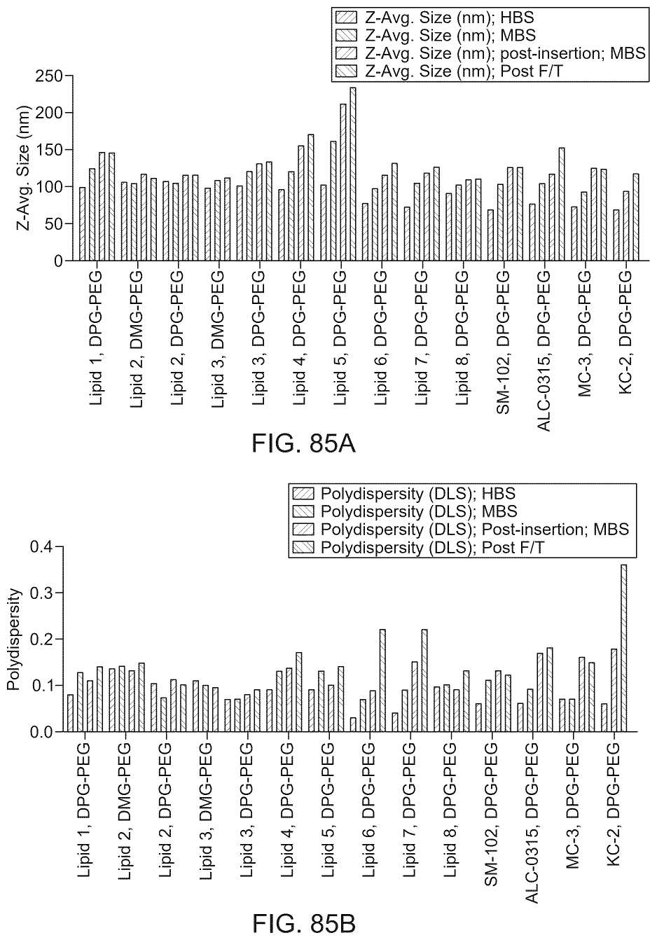

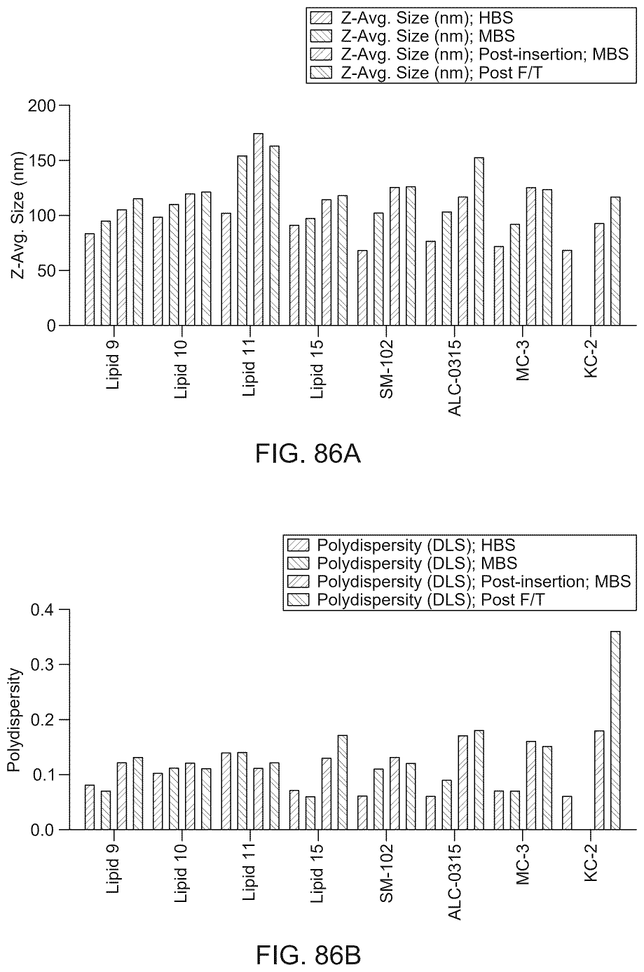

Lipids 1, 2, 3, 4, 5, 6, 7, 8, 9, 10, 11, and 15 LNPs encapsulating GFP-mRNA (TriLink Biotechnologies Inc.) were prepared and characterized as described (see Methods Section). As seen in FIG. 85A and FIG. 86A LNP diameter increased incrementally upon buffer exchange from HBS (pH 7.4) into MBS (pH 6.5), as well as upon insertion of antibody conjugate (via 4 h incubation at 37° C.) and after 1× freeze-thaw cycle. The magnitude of the upward shift in LNP diameter was lipid dependent suggesting a relationship between LNP colloidal stability and ionizable lipid chemistry. Lipid 2, 3, and 8 LNPs exhibited only small changes in particle diameter during the processing and storage steps, suggesting that nine and ten carbon (C9 and C10)N-acyl substitution constitutes the most optimal head group composition in lipids based on di-linoleoyl O-acyl lipid tails. Additionally, comparing lipids 8 to 1 (both C10 N-acyl substituted), the β-ethyl substitution (to the N-acyl group in lipid 8) resulted in better size stability relative to α-ethyl substitution (in lipid 1). Both smaller (less hydrophobic, e.g., C8 N-acyl substitution of lipid 5) and larger (more hydrophobic, i.e., C11 N-acyl substitution of lipids 6 and 7) resulted in a larger shift in LNP size indicating loss of LNP colloidal stability relative to the C9 and C10 analogs. C11 N-acyl group (lipids 6 and 7) also exhibited a lower zeta potential value at pH 5.5 relative to all other lipids consistent with lower LNP apparent pKa value (Supplemental Tables 1 and 2). This downward shift in LNP apparent pKa is attributed to more pronounced neighboring group effects with increasing hydrophobicity of the N-acyl substituent. Furthermore, the optimum head group chemistry was dependent on lipid tail group composition. A nine-carbon (C9)N-acyl substituent in lipid 4 (bearing oleoyl lipid tail groups) exhibited more significant LNP size shifts relative to size changes observed in lipids 2 and 3 LNPs (bearing linoleoyl tail groups).

SUPPLEMENTAL TABLE 1
LNP charge (Zeta Potential) of Lipids 1-8.
Charge (Zeta Potential) (DLS, mV) in pH 5.5 MES and pH
7.4 HBS of LNPs with Lipids 1-8 and comparator Lipids
SM-102, ALC-0315, MC3, and KC3, respectively.

| | LNP charge (ZP, | LNP charge (ZP, |
|---|---|---|
| Ionizable Lipid, LNP no. | mV); pH 5.5 | mV); pH 7.4 |
| Lipid 1, DPG-PEG | 19.9 | −0.212 |
| Lipid 2, DMG-PEG | 25.3 | 3.64 |
| Lipid 2, DPG-PEG | 23.1 | 2.03 |
| Lipid 3, DMG-PEG | 21.7 | 1.18 |
| Lipid 3, DPG-PEG | 19.6 | −2.01 |
| Lipid 4, DPG-PEG | 21.6 | 1.16 |
| Lipid 5, DPG-PEG | 19.1 | −0.742 |
| Lipid 6, DPG-PEG | 13.5 | −1.9 |
| Lipid 7, DPG-PEG | 10.8 | −4.31 |
| Lipid 8, DPG-PEG | 20.6 | 0.051 |
| SM-102, DPG-PEG | 12.8 | −2.25 |
| ALC-0315, DPG-PEG | 4.25 | −2.95 |
| MC-3, DPG-PEG | 12.7 | −0.43 |
| KC-2, DPG-PEG | 22.7 | 1.41 |

SUPPLEMENTAL TABLE 2
LNP charge (Zeta Potential) of Lipids 9, 10, 11, and 15.
Charge (Zeta Potential) (DLS, mV) in pH 5.5 MES and
pH 7.4 HBS of LNPs with Lipids 9, 10, 11, and 15 and
comparator Lipids SM-102, ALC-0315, MC3, and KC3, respectively.

| | LNP charge (ZP, | LNP charge (ZP, |
|---|---|---|
| Ionizable Lipid, LNP no. | mV); pH 5.5 | mV); pH 7.4 |
| Lipid 9 | 17.2 | 0.0437 |
| Lipid 10 | 21 | −0.137 |
| Lipid 11 | 16.5 | −1.01 |
| Lipid 15 | 19.7 | −0.322 |
| SM-102 | 12.8 | −2.25 |
| ALC-0315 | 4.25 | −2.95 |
| DLn-MC3-DMA | 12.7 | −0.43 |
| DLn-KC2-DMA | 22.7 | 1.41 |

Introduction of a biodegradable ester linkage in the lipid head group (lipids 9, 10, 11 and 15) resulted in a stabilizing effect on LNP colloidal stability. N-acyl substituents derived from succinate linked branched aliphatic moieties (C8 of lipid 10 and C10 of lipids 9 and 15) resulted in colloidally stable particles. However, the α-hydroxyl substituted ester analog (in lipid 11) resulted in significant loss of LNP size stability and final (post freeze-thaw) LNP diameters>150 nm. All lipids (except lipids 6 and 7) showed Polydispersity values<0.2 suggesting that the greater hydrophobicity of the head group in lipids 6 and 7 (both C11 N-acyl substituted) drives LNP aggregation upon freeze-thaw. As shown in Supplemental Tables 3 and 4 all novel lipids tested resulted in high (>80%) RNA encapsulation and good process recoveries. Supplemental Tables 1 and 2 summarize the LNP charge values, all ionizable lipids showed a relatively strong positive charge at acidic pH (5.5) and near neutral charge at physiological pH (as expected).

SUPPLEMENTAL TABLE 3
RNA content of Lipids 1-8.
Dye Accessible RNA and total RNA content of LNPs with Lipids 1-8
and comparator Lipids SM-102, ALC-0315, MC3, and KC3, respectively.

| | Nominal | Measured | Dye-accessible | TotalmRNA | Dye-accessible |
|---|---|---|---|---|---|
| Ionizable Lipid, | mRNA Conc. | Total mRNA | mRNA | Recovery | mRNA |
| LNP no. | (μg/mL) | (μg/mL) | (μg/mL) | (%) | (%) |
| Lipid 1, DPG-PEG | 100 | 75.5 | 8.9 | 75.5 | 11.8 |
| Lipid 2, DMG-PEG | 100 | 134.7 | 11.1 | 134.7 | 8.2 |
| Lipid 2, DPG-PEG | 100 | 136.9 | 9.4 | 136.9 | 6.9 |
| Lipid 3, DMG-PEG | 100 | 83.2 | 8.1 | 83.2 | 9.8 |
| Lipid 3, DPG-PEG | 100 | 93.5 | 10.3 | 93.5 | 11.0 |
| Lipid 4, DPG-PEG | 100 | 77.6 | 9 | 77.6 | 11.6 |
| Lipid 5, DPG-PEG | 100 | 73.4 | 11.8 | 73.4 | 16.1 |
| Lipid 6, DPG-PEG | 50 | 34.7 | 3.3 | 69.4 | 9.5 |
| Lipid 7, DPG-PEG | 50 | 38.9 | 4.3 | 77.8 | 11.1 |
| Lipid 8, DPG-PEG | 100 | 85.9 | 8.4 | 85.9 | 9.8 |
| SM-102, DPG-PEG | 50 | 28.7 | 4.3 | 57.4 | 15.0 |
| ALC-0315, DPG-PEG | 50 | 38.1 | 4.8 | 76.2 | 12.6 |
| MC-3, DPG-PEG | 50 | 36.8 | 1.8 | 73.6 | 4.9 |
| KC-2, DPG-PEG | 50 | 43 | 3.6 | 86 | 8.4 |

SUPPLEMENTAL TABLE 4
RNA content of Lipids 9, 10, 11, and 15.
Dye Accessible RNA and total RNA content of
LNPs with Lipids 9, 10, 11, and 15, respectively,

| | Nominal | Measured | Dye- | Total | Dye- |
|---|---|---|---|---|---|
| Ionizable | mRNA | Total | accessible | mRNA | accessible |
| Lipid, | Conc. | mRNA | mRNA | Recovery | mRNA |
| LNP no. | (μg/mL) | (μg/mL) | (μg/mL) | (%) | (%) |
| Lipid 9 | 100 | 84.4 | 5.4 | 84.4 | 6.4 |
| Lipid 10 | 100 | 80.2 | 6.6 | 80.2 | 8.2 |
| Lipid 11 | 100 | 85.6 | 13.6 | 85.6 | 15.9 |
| Lipid 15 | 100 | 74.9 | 5.1 | 74.9 | 6.8 |

In addition to the α-CD3 targeted LNPs described above, α-CD8 targeted LNPs based on lipids 3, 4, 5, 9, and 15 were produced by post-insertion (using a 4-hour incubation at 37° C.) of two α-CD8 targeting conjugates, namely, a TRX2 (an α-CD8 Fab conjugated to the PEG terminus of DSPE-PEG2k) and Nb8 (an α-CD8 VHH conjugated to the PEG terminus of DSPE-PEG3.4K). The resulting particle size characteristics (post-insertion and post freeze-thaw) are summarized in Supplemental Table 5. LNP diameters after the initial mixing step and subsequent buffer exchange into HBS measured≤120 nm with Lipids 3, 9 and 15, while Lipid 5 LNPs exhibited>150 nm diameter. Notably, lipids 9 and 15 LNPs exhibited the smallest sizes (≤120 nm, PDI<0.2) after both αCD8 antibody conjugate insertions (TRX2 Fab conjugate and the Nb8 VHH conjugate) and after one freeze-thaw cycle, suggesting more robust LNP colloidal stability relative the LNPs based on lipids 3, 4 and 5. Thus introduction of a biodegradable succinate derived N-acyl substituent helps improve LNP colloidal stability under processing and freeze-thaw stress.

SUPPLEMENTAL TABLE 5
LNP size and PDI of targeted LNPs.
Size and PDI of LNPs with Lipids 3, 4, 5, 9, and 15, pre- and post-
insertion with αCD8 (TRX2 Fab and Nb8 V_{HH}) targeting conjugates.

| | Z-Avg. | Z-Avg. | Z-Avg. Size | Z-Avg. Size | | | PDI (DLS); | PDI (DLS); |
|---|---|---|---|---|---|---|---|---|
| Ionizable | Size | Size | (nm); TRX2 | (nm); Nb8 | PDI | PDI | TRX2 Post- | Nb8; Post- |
| Lipid, LNP | (nm); | (nm); | post-insertion; | post-insertion; | (DLS); | (DLS); | insertion; | insertion; |
| no. | HBS | MBS | MBS | MBS | HBS | MBS | MBS | MBS |
| Lipid 3, | 101.5 | 110.8 | 117.4 | 131.0 | 0.08 | 0.09 | 0.116 | 0.166 |
| DPG-PEG, | | | | | | | | |
| TRX2 | | | | | | | | |
| Lipid 9, | 83.9 | 94.8 | 102.5 | 108.1 | 0.08 | 0.07 | 0.1255 | 0.143 |
| DPG-PEG, | | | | | | | | |
| TRX2 | | | | | | | | |
| Lipid 4, | 95.5 | 120.6 | 127.8 | 133.7 | 0.09 | 0.13 | 0.1325 | 0.114 |
| DPG-PEG, | | | | | | | | |
| TRX2 | | | | | | | | |
| Lipid 5, | 102.4 | 161.2 | 162.3 | 161.3 | 0.09 | 0.13 | 0.1335 | 0.095 |
| DPG-PEG, | | | | | | | | |
| TRX2 | | | | | | | | |
| Lipid 15; | 91.55 | 97.65 | 106.0 | 115.1 | 0.07 | 0.06 | 0.1415 | 0.164 |
| DPG-PEG, | | | | | | | | |
| TRX2 | | | | | | | | |

### Physiochemical Properties of Targeted LNPs Based on Lipids 10, 15, 16, 24A, 26 and ALC-0315

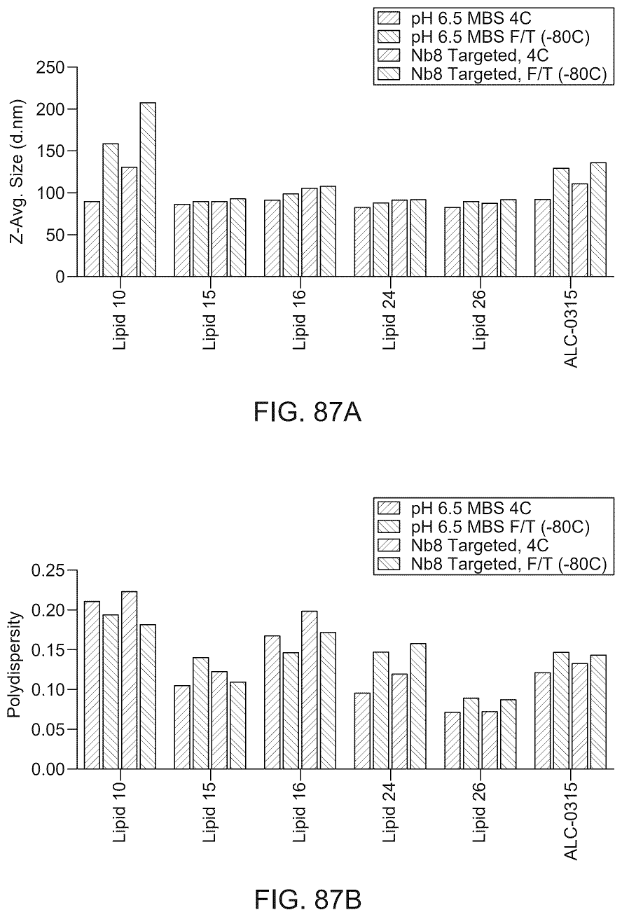

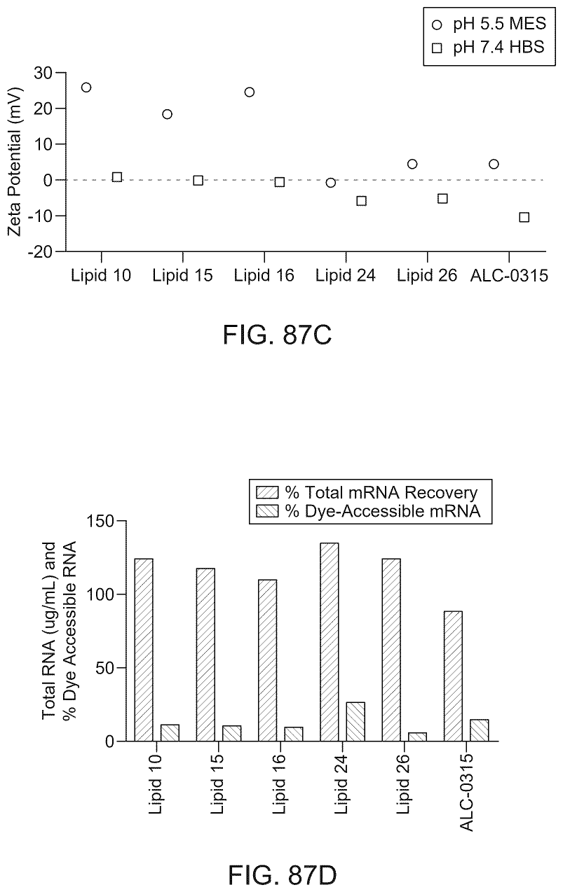

LNP size, polydispersity, charge, and RNA content values of Lipids 10, 15, 16, 24A, 26, and ALC-0315 LNPs are summarized in Supplemental Information (Supplemental Tables 2, 6, 7, and 8 and FIG. 87A-FIG. 87D). As seen in FIG. 87A, all parent formulations based on Lipids 10, 15, 16, 24A and 26 resulted in LNP diameter<100 nm in pH 6.5 MES buffer. Lipid 10 and ALC-0315 LNPs exhibited a notable size increase upon freeze-thaw (with 10% sucrose in MES pH 6.5 buffer), while a minimal impact of one Freeze-Thaw cycle on LNP diameter was observed in Lipid 15, 16, 24A, and 26 LNPs. Similarly, relatively small changes in LNP diameter were observed upon targeting antibody insertion (in pH 6.5 MES using a 37° C. incubation for 4 hours) for Lipids 15, 16, 24A, and 26, while larger increase in LNP diameter was observed for Lipid 10 and ALC-0315 LNPs. The corresponding targeted LNPs also exhibited a similar trend in size changes upon one Freeze Thaw cycle. All LNPs exhibited moderate to high encapsulation efficiency (<15% Dye accessible RNA), except for ALC-0315 LNPs, where higher dye accessible RNA was observed. Also, lipid 10, 15 and 16 LNPs exhibited strongly positive charge in acidic pH (5.5), while near neutral charge in physiological pH (7.4). In contrast, Lipids 24A, 26 an ALC-0315 LNPs exhibited a relatively weak positive charge in acidic pH (5.5) and a slight negative charge at physiological pH (7.4). The negative charge under physiological pH conditions (seen in LNPs based on lipids 14A, 26 and ALC-0315) suggests that the branched tail groups in this subset of lipids alters the interfacial presentation of the ionizable head group, thereby driving LNP apparent pKa towards lower values relative to the linoleic and oleic acid tail groups of lipids 10 and 15, respectively.

SUPPLEMENTAL TABLE 6
LNP size and PDI of targeted LNPs.
Size and PDI of LNPs with Lipids 10, 15, 16, 24A, 26, and ALC-
0315, pre- and post-insertion with Nb8 V_{HH} targeting conjugate.

| Ionizable | Z-Avg. | Z-Avg. Size | Z-Avg. | | | |
|---|---|---|---|---|---|---|
| lipid, LNP | Size (nm); | (nm); post- | Size (nm); | Polydispersity | Polydispersity (DLS); | Polydispersity |
| no. | MBS | insertion; MBS | Post F/T | (DLS); MBS | Post-insertion; MBS | (DLS); Post F/T |
| Lipid 10, | 90 | 131 | 208 | 0.21 | 0.22 | 0.18 |
| DPG-PEG | | | | | | |
| Lipid 15, | 86 | 90 | 94 | 0.11 | 0.12 | 0.11 |
| DPG-PEG | | | | | | |
| Lipid 16, | 92 | 106 | 109 | 0.17 | 0.20 | 0.17 |
| DPG-PEG | | | | | | |
| Lipid 24A, | 82 | 91 | 94 | 0.10 | 0.12 | 0.16 |
| DPG-PEG | | | | | | |
| Lipid 26, | 83 | 87 | 93 | 0.07 | 0.07 | 0.09 |
| DPG-PEG | | | | | | |
| ALC-0315, | 93 | 111 | 136 | 0.12 | 0.13 | 0.14 |
| DPG-PEG | | | | | | |

SUPPLEMENTAL TABLE 7
LNP charge (Zeta Potential) of Lipids 10, 15, 16, 24A, and 26.
Charge (Zeta Potential) (DLS, mV) in pH 5.5 MES and pH
7.4 HBS of LNPs consisting of Ionizable Lipids 10, 15, 16,
24A, and 26 and comparator Lipid ALC-0315, respectively.

| Ionizable lipid, | Charge (ZP, mV); | Charge (ZP, mV); |
|---|---|---|
| LNP no. | pH 5.5 | pH 7.4 |
| Lipid 10, DPG-PEG | 25.8 | 0.8 |
| Lipid 15, DPG-PEG | 18.4 | −0.2 |
| Lipid 16, DPG-PEG | 24.5 | −0.6 |
| Lipid 24A, DPG-PEG | −0.7 | −5.9 |
| Lipid 26, DPG-PEG | 4.3 | −5.2 |
| ALC-0315, DPG-PEG | 4.4 | −10.5 |

SUPPLEMENTAL TABLE 8
RNA content of Lipids 10, 15, 16, 24A, and 26.
Dye Accessible RNA and total RNA content
of LNPs consisting of Ionizable Lipids
10, 15, 16, 24A, and 26, respectively.

| | Nominal | Measured | Dye- |
|---|---|---|---|
| Ionizable lipid, | mRNA | Total | accessible |
| LNP no. | Conc. (μg/mL) | mRNA (μg/mL) | mRNA (%) |
| Lipid 10, DPG-PEG | 150 | 124.30 | 10.9 |
| Lipid 15, DPG-PEG | 150 | 117.60 | 10.3 |
| Lipid 16, DPG-PEG | 150 | 109.90 | 9.40 |
| Lipid 24A, DPG-PEG | 150 | 134.90 | 26.4 |
| Lipid 26, DPG-PEG | 150 | 124.40 | ≤6 (5.5) |
| ALC-0315, DPG-PEG | 150 | 88.50 | 14.70 |

Effect of Lipid Head Group Hydrophobicity and Regio-Isomerism on In Vitro T-Cell Transfection with Targeted LNPs Based on Lipids 1, 3, 5, and 8.

αCD3 (SP34 Fab) targeted GFP mRNA LNPs based on lipids 1, 3, 5, and 8 were dosed to primary human T-cells at dose levels between 0.001 and 1 μg/mL in two independent experiments (each experiment included LNPs based on a comparator lipid DLn-KC2-DMA) and GFP expression was assessed by Flow Cytometry analysis at 24 hours. As seen in FIG. 88A and FIG. 8B and FIG. 89A and FIG. 89B, the percent GFP+ cells and the Mean Fluorescence Intensity (MFI) of transfected T-cells suggested that hydrophobicity of the N-acyl substituent impacts protein expression. Lipids 5 and 3 feature an 8 carbon, 9 carbon N-acyl group respectively, while lipids 1 and 8 feature a 10-carbon N-acyl substituent (α-ethyl octanoic acid in lipid 1 vs β-ethyl octanoic acid in lipid 8). The order of GFP expression levels observed was Lipid 5 LNP>Lipid 3 LNP>1 LNP. This indicates that the least hydrophobic N-acyl substituent (C8) resulted in the most efficient cytosolic delivery of the mRNA payload and a step wise drop in the efficiency result with C9, C10 substitution (of Lipid 3 and 1, respectively). This trend suggests that greater hydrophobicity of the N-acyl substituent either limits partitioning (and fusogenicity) into endosomal membranes or slows RNA release (dissociation of ionizable lipid from RNA) in the cytoplasm thereby limiting the availability of the RNA payload for ribosome engagement and protein translation.

Additionally, the β-ethyl octanoic acid substitution (in lipid 8) significantly improves LNP activity compared to the α-ethyl octanoic acid substitution (in lipid 1) indicating an effect of positional isomerism. The improved performance of the β-ethyl octanoic acid substitution (in lipid 8) over α-ethyl octanoic acid substitution (in lipid 1) may be attributed to enhanced aliphatic hydrocarbon mobility (lower melting point) and enhanced fusogenicity towards anionic endosomal membranes.

With respect to the optimal lipid composition for RNA delivery to T-cells, both LNP colloidal stability and LNP activity were considered. As described earlier, the 8-carbon N-acyl substitution (of lipid 5) was the most active lipid (resulting in the highest protein expression). However, Lipid 5 LNPs exhibited inferior size stability relative to the 9C (lipid 3) and 10C compositions (lipids 1 and 8) LNPs. Thus, considering both factors, the 9-carbon N-acyl composition (of lipid 3) or β-substituted 10C compositions were found optimal.

Effect of Head Group Hydrophobicity on In Vitro T-Cell Transfection with Targeted LNPs Based on Lipids Derived from a Biodegradable Ester-Linked N-Acyl Substituent

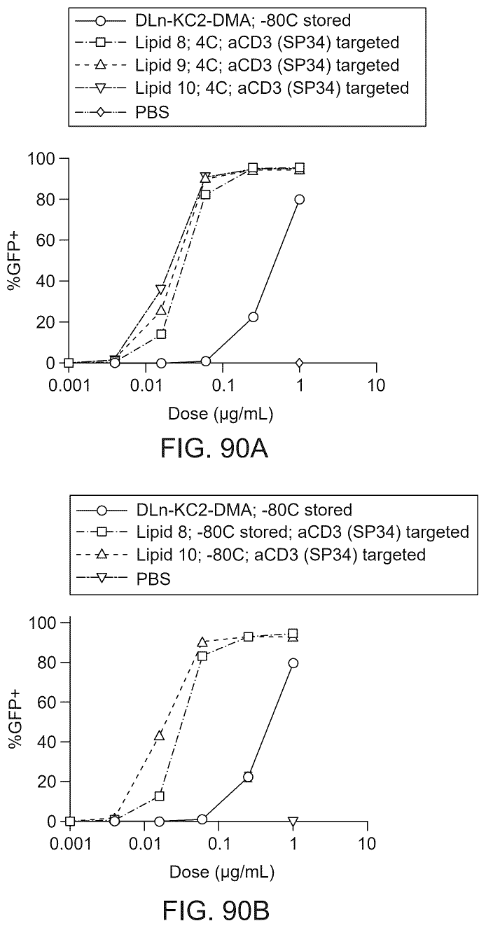

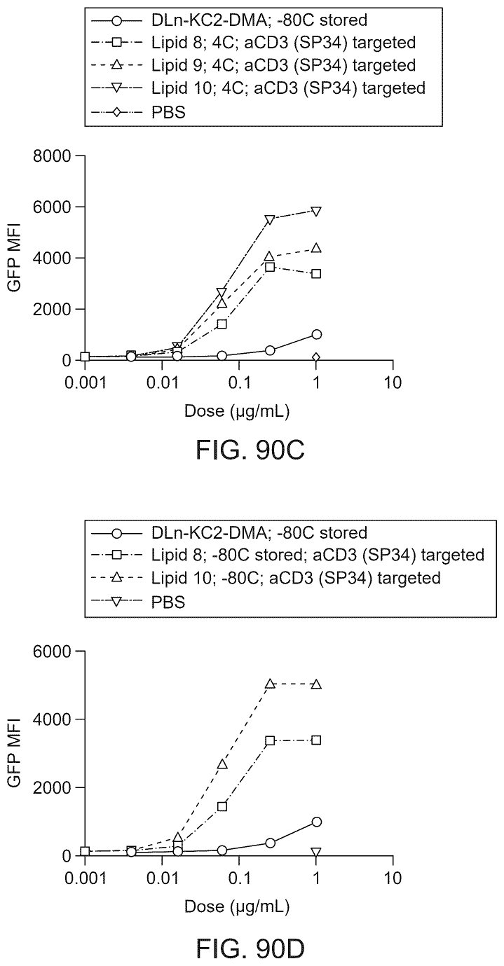

Lipids 10 and 9 were designed to introduce a biodegradable ester linkage in the N-acyl substituent. Based on optimal hydrophobic content and effects of β-substitution described above, either an 8- or a 10-carbon (C8 or C10) aliphatic ester of 3-octanol (in lipid 10) or 4-decanol (in lipid 9) and succinic acid were chosen. The in vitro protein expression levels (in primary human T-cells) resulting from αCD3 targeted, GFP mRNA LNPs based lipids 10 and 9 was compared to the expression levels from lipid 8 LNPs (a 10 carbon, β-substituted N-acyl composition without the succinate ester linkage) and a comparator lipid DLn-KC2-DMA. As seen in FIG. 90C, GFP expression in T-cells ranked in the order lipid 10>lipid 9>lipid 8 indicating that succinate ester improves LNP performance. Furthermore, improved performance of lipid 10 LNPs over Lipid 9 LNPs indicates that the 3-octanol derived succinate ester results in improved expression over the 4-decanoate derived analog consistent with the trends in N-acyl substituent hydrophobicity observed between lipid 5 (8C) and lipid 3 (9C) LNPs as described above.

Effect of Lipid Tail Unsaturation on In Vitro T-Cell Transfection with Targeted LNPs Derived from Linoleic Acid and Oleic Acid Lipid Tail Groups

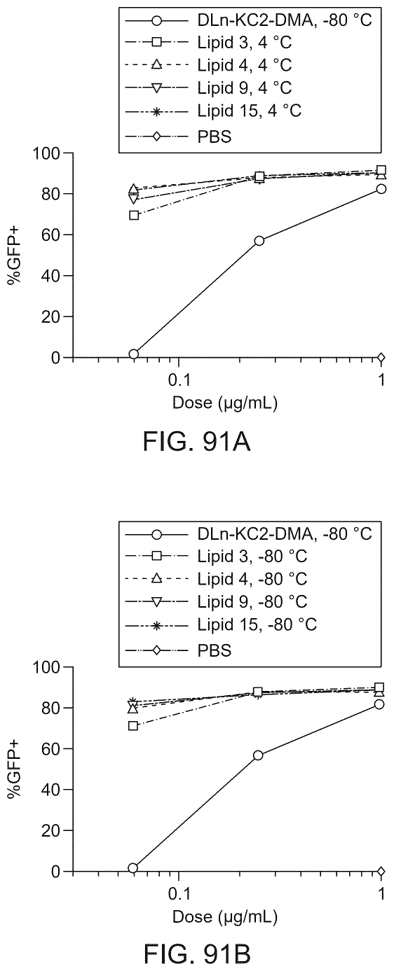

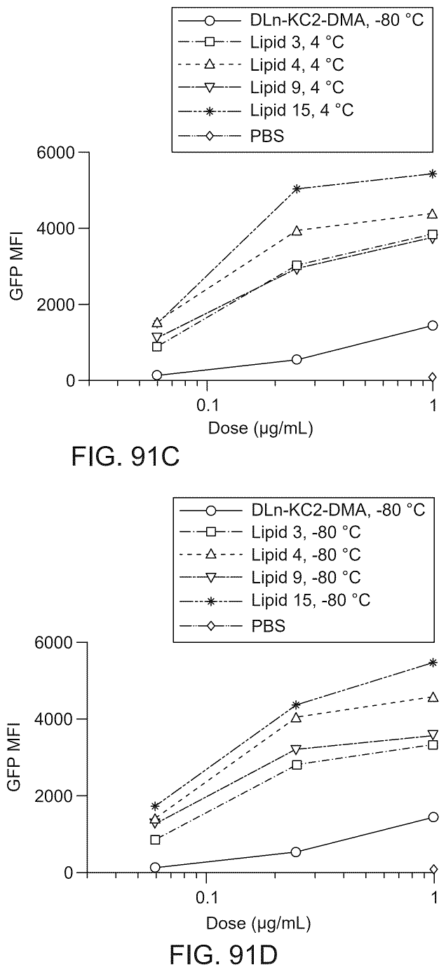

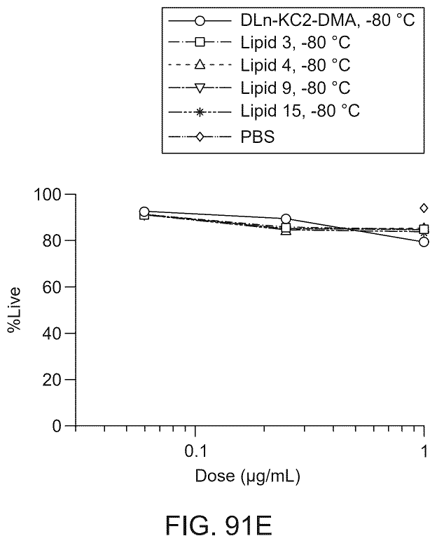

αCD3 (SP34 Fab) targeted GFP mRNA LNPs based on lipids 3, 4, 9, and 15 were dosed to primary human T-cells at dose levels between 0.001 and 1 μg/mL (alongside comparator lipid, DLn-KC2-DMA LNPs) either after storage at 4C or after being stored frozen at −80C and thawed at room temperature prior to dosing. GFP expression was assessed by Flow Cytometry analysis at 24 hours. As seen in FIG. 91, at all dose levels, performance improvements (both in terms of fraction of GFP+ T-cells as reflected by % GFP+ cells and the copies of GFP protein produced on a per cell basis as reflected by GFP MFI values) were observed with lipid 4 (relative to lipid 3) and with lipid 15 (relative to lipid 9) indicating that the oleoyl tail groups (in Lipids 4 and 15) result in higher levels of protein expression over the corresponding linoleoyl tail groups (in Lipids 3 and 9) in the context of T-cell targeted lipid nanoparticles. Ionizable lipids derived from linoleic acid tail (di-unsaturated) groups are reported to improve protein expression relative to lipids derived from either oleic acid (mono-unsaturated) or saturated fatty acid tails. Linoleic acid-derived lipid tails are expected to lower lipid melting point (Tm) and favor phase transition of the lipid bilayer towards an inverted hexagonal Hu phase via ionizable lipid fusion with anionic lipids of the endosomal membranes. Thus, promoting endosomal membrane disruption and improved cytosolic delivery of the RNA payload. However, the phenomenon is not universal as illustrated here by the improved performance of lipid 5 (over lipid 4) and of lipid 15 (over lipid 9) LNPs. Furthermore, in contrast to Lipid 4 where poorer LNP size distribution properties were observed (relative to Lipid 3), Lipid 15 LNPs exhibited size distribution properties similar to those observed with Lipid 9 LNPs. Thus, while the di-oleoyl tail group does not affect size stability in the context of the decan-4-ol succinate N-acyl substituent (of lipid 15), it adversely impacted size stability in lipid 4 in the context of the 3-carboxyoctane derived N-acyl substituent. This suggests that the LNP size distribution (and in-process size stability) is dependent both on the chemistry of lipid tail group as well as of the N-acyl substituent. The performance levels and relative order of LNP efficiency were well preserved in all formulations tested after 1 Freeze-Thaw cycle (Post-80° C. storage) as illustrated by the comparison of % GFP+ cells and GFP MFI values before and after −80° C. storage (FIG. 91A versus 11B and 11C versus 11D. Lipids 3, 4, 9, and 15 LNPs were well tolerated by primary human T-cells (FIG. 91E).

Effect of Lipid Tail Unsaturation on In Vitro T-Cell Transfection with LNPs Based on Lipids 3, 4, 9, and 15, and Evidence of Receptor-Mediated Uptake into Primary Human T-Cells

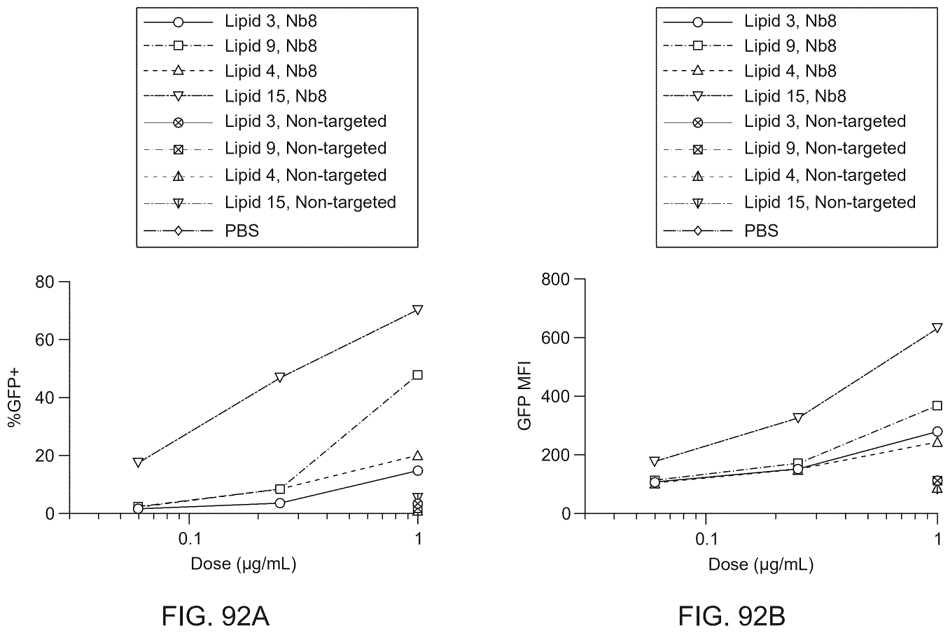

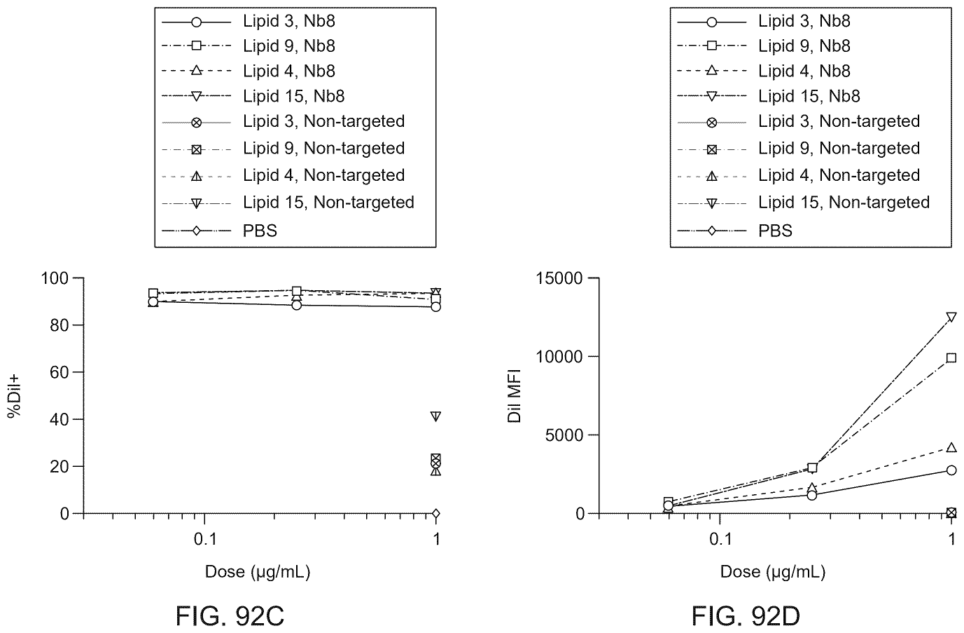

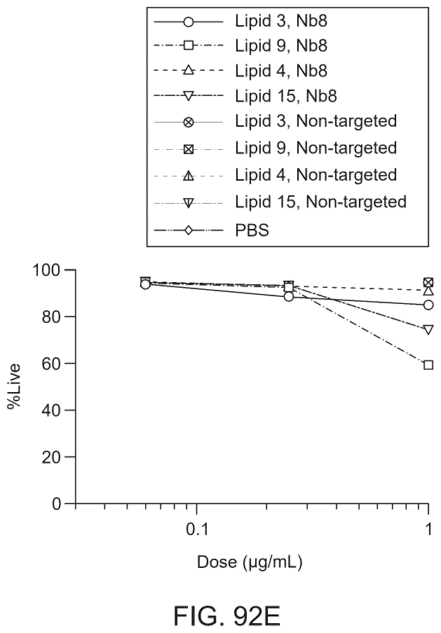

Parent LNPs based on lipids 3, 4, 9, and 15 as well the corresponding targeted LNPs resulting from post insertion of αCD8 VHH Nb8 were dosed to primary human T-cells at dose levels between 0.001 and 1 μg/mL, and GFP expression was assessed by Flow Cytometry analysis at 24 hours. As seen in FIGS. 92A and 92B, at all dose levels, Lipid 4 and Lipid 15 outperformed Lipid 3 and Lipid 9 LNPs, consistent with the results described above with the αCD3 SP34 Fab targeted LNPs. Additionally, parent LNPs (dosed at 1 μg/mL only) resulted in no significant expression thus confirming the role of receptor-mediated uptake into T-cells. Lipids 3, 4, 9, and 15 αCD8 VHH Nb8 targeted LNPs were well tolerated by primary human T-cells (FIG. 92E) with cell viability trending measurably lower in Lipid 9 and Lipid 15 formulations possibly due to the higher levels of GFP protein expression (as indicated by the higher GFP MFI values seen FIG. 92B).

Effect of Tail Group Composition (Unsaturation and Branching) on In Vitro GFP Protein Expression in Primary Human T-Cells Transfected with αCD8 (15C01) Targeted LNPs Based on Lipids 10, 15. 16, 24A, 26, and ALC-0315

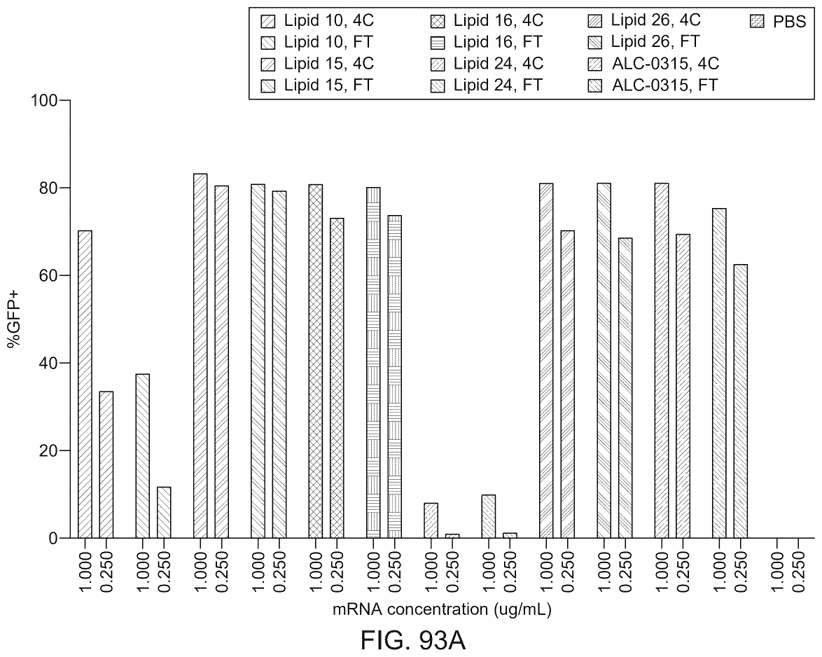

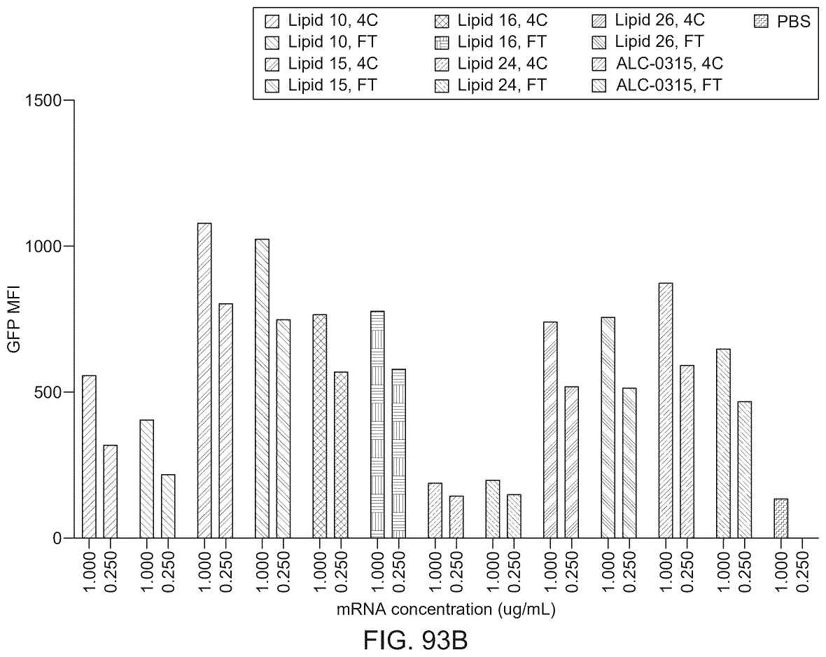

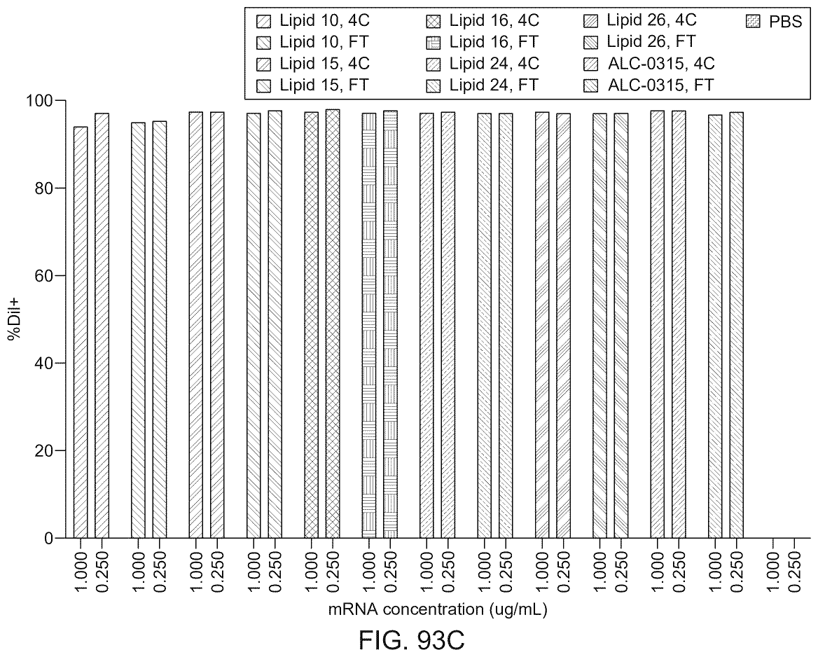

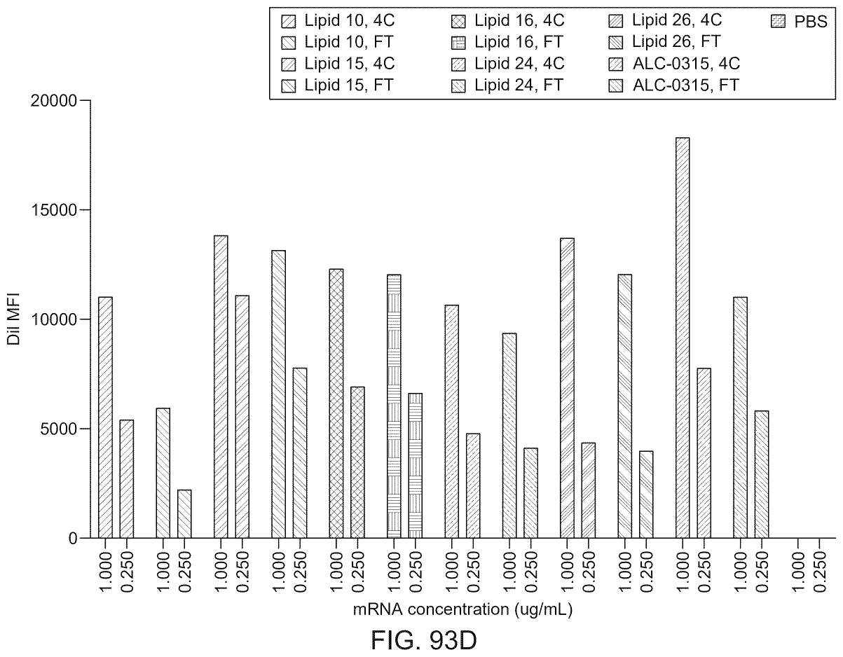

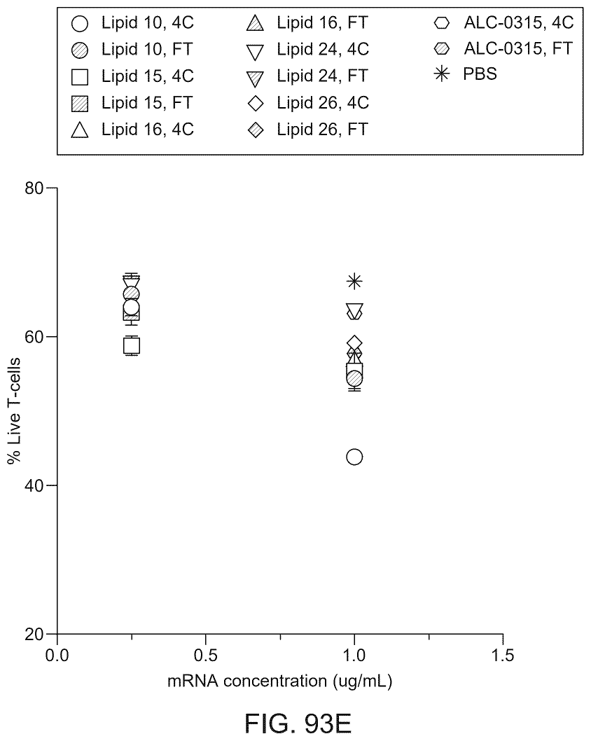

Lipids 10, 15, 16, 24A, 26, and ALC-0315, αCD8 (Nb8 VHH) targeted LNPs were well tolerated by primary human T-cells (FIG. 93E). As seen in FIG. 93C and FIG. 93D, αCD8 Nb8 targeted LNPs based on lipids 10, 15, 16, 24A, 26, and ALC-0315 showed similar high % DiI+ (dye) T-cells and DiI MFI reflective of equally efficient LNP association with the CD8+ T-cell population. However, notable differences in GFP protein expression levels (as reflected by % GFP+ cells and the copies of GFP protein produced on a per cell basis as reflected by GFP MFI values) were observed (FIG. 93A and FIG. 93B). Lipid 15 LNPs outperformed the other Lipids within the group, with Lipids 26 and ALC-0315 also enabling relatively high levels of protein expression. It is noteworthy that Lipid 15 and Lipid 16 LNP Zeta Potential values at pH 5.5 and pH 7.4 suggest a large shift in charge and consequently a strong ability for fusion with endosomal membranes (and endosome disruption/endosomal escape) upon acidification of the endosomal compartment, However, Lipid 26 and ALC-0315 LNP Zeta Potential values suggest a relatively small charge shift under endosomal acidification. Hence, despite potentially a lower ability for charge-driven endosomal membrane destabilization, Lipid 26 and ALC-0315 LNPs enabled high levels of protein expression reflective of potent cytosolic delivery of the GFP RNA payload. This suggests that the more pronounced “cone” structure of the branched tail group and greater lipid cross-sectional area (in lipids 26 and ALC-0315) favors lipid bilayer transition to the Hexagonal Hu phase upon ionizable lipid fusion with negatively charged endosomal membrane lipids. Thus, enabling efficient endosomal membrane disruption, cytosolic availability of the RNA payload, and higher levels of protein expression.

### Jurkat Activation Assay

The human T-cell line, Jurkat, Clone E6-1, was purchased from ATCC and was maintained according to the manufacturer's recommendations. Jurkat cells were transfected with CAR-encoding mRNA by electroporation. Briefly, 105 Jurkat cells were resuspended in 20 μL SE 4D-Nucleofector™ X Solution (Lonza) and mixed with 0.6 μg mRNA. The cells were then electroporated in 20 μL Nucleocuvette strips using a 4D-Nucleofector X Unit (Lonza). Following a 2-hour rest period post electroporation, Jurkat cells were co-cultured with Raji cells at effector-to-target cell ratios of 4:1. 24 hours after co-culture, cells were stained with CD3-APC (BD Biosciences) and CD69-BV421 (BD Biosciences). Dead cells were excluded from the analysis by using eFluor780 fixable viability dye (Thermo Fisher). CAR-mediated activation was defined as the CD69 MFI level in live CD3⁺ cells (signal) divided by the CD69 MFI level in a group without Raji cells (background).

### Cytotoxicity Against Additional Cell Lines and Quantification of Surface Markers

HT, Namalwa, EB-1, Toledo, Su-DHL-6, and Su-DHL-8 cells (Supplemental Table 9) were stained with Cell Trace Violet proliferation dye (CTV) (Thermo Fisher) following the manufacturer's protocol. Briefly, target cells were incubated with CTV for 20 minutes at room temperature prior to washing and co-culturing. CD8⁺ T-cells were isolated and transfected as described above and washed after 2 hours of incubation to remove unbound LNPs. Transfected CD8⁺ T-cells were then co-cultured with CTV-stained target cells at varying effector-to-target cell ratios in T-cell media (RPMI-1640, 10% FBS, and 50 ng/mL recombinant human IL-2) in a 96-well flat-bottom plate for 48 hours. After 48 hours of co-culture, cells were stained with eFluor780 fixable viability dye (Thermo Fisher) to assess the level of cytotoxicity. Cells were washed twice in FACS buffer and acquired by Flow Cytometry on a Penteon Novocyte (Agilent) and further analyzed in FlowJo, where dead target cells were defined as CTV⁺ and Live/Dead eFluor780⁺ cells.

A Quantibrite PE kit (BD Biosciences) was used to quantify the number of CD19 and CD22 molecules per cell, respectively, on each cell line following the manufacturer's instructions.

SUPPLEMENTAL TABLE 9
Receptor quantification and EC50 values in various target
cell lines (CD19 and CD22 expression density and FMC63
and 22-V4 cytotoxicity in selected target cell lines)

| | FMC63 | 22-V4 | CD19 | CD22 | |
|---|---|---|---|---|---|
| Cell line | EC50 | EC50 | density | density | Media |
| Nalm6 | 0.89 | 1.04 | 35740 | 2741 | RPMI 10% FBS |
| Raji | 6.78 | 8.91 | 57232 | 10322 | RPMI 10% FBS |
| Daudi | 4.97 | 8.86 | 45494 | 18482 | RPMI 10% FBS |
| JVM-2 | 13.14 | 13.80 | 12871 | 290 | RPMI 10% FBS |
| Reh | 1.40 | 1.64 | 4966 | 618 | RPMI 10% FBS |
| K562 | — | — | 33 | 48 | RPMI 10% FBS |
| HT | 4.49 | 6.05 | 38031 | 9416 | RPMI 10% FBS |
| Namalwa | 2.46 | 2.38 | 34793 | 507 | RPMI 10% FBS |
| EB-1 | 3.15 | 5.37 | 39006 | 2490 | RPMI 10% FBS |
| Toledo | 1.31 | 1.24 | 22730 | 366 | RPMI 10% FBS |
| Su-DHL-6 | 3.36 | 3.07 | 13469 | 6098 | RPMI 10% FBS |
| Su-DHL-8 | 0.81 | 0.81 | 40960 | 573 | RPMI 10% FBS |

### Immunohistochemistry

Formalin-fixed samples of livers and spleens collected from mice on day 26 of the efficacy study were processed and paraffin-embedded. Staining for immunohistochemistry (IHC) was done using a mouse anti-human monoclonal antibody (Abcam; BLCAM/1795 clone) with specificity for the human B-lymphocyte marker, CD22. All slides were subjected to histopathology evaluation. Slides were blindly evaluated by a board-certified veterinary pathologist. Quantitative analysis for hCD22 IHC was performed on whole slide digital images within the HALO Indica Labs image analysis software platform with the Cytonuclear v2 algorithm. Individual mouse scores are shown in Supplemental Tables 10 and 11.

SUPPLEMENTAL TABLE 10
Liver Immunohistochemistry on Day 26 of efficacy study (Table showing the tissue
area, total number of cells, number of human CD22-positive cells, number of human
CD22-positive cells per mm2, and fraction of human CD22-positive cells of the
total number of cells in the liver of n = 3 mice from each treatment group).

| | | Mouse | Tissue Area | Total | hCD22+ | hCD22+ | % hCD22+ |
|---|---|---|---|---|---|---|---|
| Tissue | Treatment | ID | (mm²) | cells | cells | cells/mm² | cells |
| Liver | Vehicle | 1-3 | 30.52 | 254790 | 879 | 29 | 0.345 |
| | | 1-4 | 21.71 | 163588 | 6673 | 307 | 4.079 |
| | | 1-5 | 20.40 | 194435 | 5674 | 278 | 2.918 |
| | LNP | 2-3 | 33.45 | 236847 | 14924 | 446 | 6.301 |
| | control | 2-4 | 19.00 | 167309 | 3556 | 187 | 2.125 |
| | | 2-5 | 22.41 | 161542 | 6885 | 307 | 4.262 |
| | 22-V4 | 3-3 | 31.56 | 254801 | 156 | 5 | 0.061 |
| | | 3-4 | 35.41 | 274243 | 3670 | 104 | 1.338 |
| | | 3-5 | 31.50 | 304747 | 343 | 11 | 0.113 |

SUPPLEMENTAL TABLE 11
Spleen Immunohistochemistry on Day 26 of efficacy study (Table showing the tissue
area, total number of cells, number of human CD22-positive cells, number of human
CD22-positive cells per mm2, and fraction of human CD22-positive cells of the
total number of cells in the spleen of n = 3 mice from each treatment group).

| | | Mouse | Tissue Area | Total | hCD22+ | hCD22+ | % hCD22+ |
|---|---|---|---|---|---|---|---|
| Tissue | Treatment | ID | (mm²) | cells | cells | cells/mm² | cells |
| Spleen | Vehicle | 1-3 | 8.347 | 81379 | 3614 | 433 | 4.441 |
| | | 1-4 | 5.929 | 61739 | 267 | 45 | 0.432 |
| | | 1-5 | 4.687 | 46155 | 706 | 151 | 1.530 |
| | LNP | 2-3 | 4.792 | 48660 | 678 | 141 | 1.393 |
| | control | 2-4 | 5.121 | 52732 | 257 | 50 | 0.487 |
| | | 2-5 | 5.327 | 56331 | 140 | 26 | 0.249 |
| | 22-V4 | 3-3 | 2.774 | 30157 | 4 | 1 | 0.013 |
| | | 3-4 | 2.883 | 29464 | 9 | 3 | 0.031 |
| | | 3-5 | 1.956 | 20344 | 1 | 1 | 0.005 |

### Amnis® ImageStream Assay

### Sample Acquisition

Samples were acquired on a Cytek® Amnis® ImageStream® X Mk II (Cytek Biosciences, Fremont, CA, USA) equipped with 2 cameras and 4 lasers. Data was acquired using Cytek® Amnis® INSPIRE™ acquisition software. The system was calibrated with ASSIST (Automated Suite of System-wide ImageStreamX Tests) using the INSPIRE software.

To maximize signal detection and avoid saturation of the cameras, the following settings were utilized: 120 mW 405 nm violet laser, 150 mW 488 nm blue laser, 150 mW 642 nm red laser and 2 mW 785 nm NIR laser (for side scatter). The images were acquired using slow speed (highest sensitivity) with a magnification of 40× (0.5 um2/pixel resolution). Bright-field imagery was collected using an LED array with wavelengths of 430 nm to 470 nm in channel 1 (brightfield 1) and 575 nm to 595 nm in channel 9 (brightfield 2). Side scatter was collected in channel 6 using the dedicated 785 nm laser.

Controls for compensation were acquired with brightfield and side scatter illumination sources off and with all imaging channels enabled.

The staining panel included Channel 1, Em Band 430-470 for Brightfield-Morphology; Channel 2, Ex 488, Em Band 470-560 for FITC-CD8; Channel 3, Ex 488/561, Em Band 560-595 for PE-CAR; Channel 6, Ex 488/561, Em Band 745−800 for SSC-Granularity; Channel 7, Ex 405, Em Band 430-505 for CellTrace Violet-Nalm6 cells; Channel 9, Ex 405, Em Band 575-595 for Brightfield-Morphology; Channel 11, Ex 642/405, Em Band 660-720 for BV711-CD3; Channel 12, Ex 642/405, Em Band 720−800 for eFluor780-Viability.

Approximately 50,000 objects were recorded per sample, based on objects displaying a larger area than the ASSIST SpeedBeads in the Area vs Aspect ratio plot of the BF channel (Ch01). For each sample, data was saved in raw image file (.rif) format then analyzed in Cytek® Amnis® IDEAS® v6.4+Machine Learning (ML) software.

### Interacting Cell Analysis Using IDEAS® v6.4+ML

IDEAS® v6.4 is a cellular analysis software for the Cytek® Amnis® ImageStream® X Mk II imaging flow cytometer. The software allows for quantification of cellular activity by performing statistical analyses on thousands of events and, at the same time, permits visual confirmation of any individual event.

The single-color compensation controls acquired during acquisition were used to correct for fluorescence emission spillover into off-target detection channels and generate a compensation matrix. The matrix was applied to the fully stained samples and the compensated data was analyzed.

Doublet cells were identified by generating a gate on “In Focus” objects (Gradient RMS Brightfield>50) and “doublet” objects (Area Brightfield>220 um2 and Aspect Ratio Brightfield<0.77). To confirm the presence of CellTrace Violet (B Cells) and CD3 doublet events, an intensity plot was created, and double positive objects were selected. Dead cells were removed from the analysis with a negative selection gate on the intensity of Live/Dead Far Red. The “In Focus”, “Doublet”, “CellTrace Violet+, CD3+”, and “Live” objects were further observed for the total intensity of CAR vs the max pixel intensity of CAR. CAR+events were identified as total intensity>1600 RFUs and max pixel intensity>97 RFUs. The final step in the analysis was to determine if the B and T cells were interacting.

The machine learning (ML) module for the Cytek® Amnis® IDEAS® software creates an experiment-specific feature that distinguishes populations based on user input. The module is built upon a modified version of the linear discriminant analysis (LDA) method and is integrated into Cytek® Amnis® IDEAS® v6.4. The module leverages available feature and masking algorithms, normalizes all features to a common base (to offset the wide variation in feature value ranges), uses Fisher's Discriminant Ratio for feature selection then uses a weighted linear combination of top-selected features to generate a linear classifier based upon input populations.

Twenty-five positive B cell-T cell interactions and 25 negative B cell-T cell interactions were individually tagged via the software tagging tool. The tagged populations were analyzed using the machine learning module while specifying “In Focus”, “Doublet”, “CellTrace Violet+, CD3+”, “Live”, “CAR+” objects as the base population. Using the B cell-T cell positive interaction classifier, objects yielding a positive value (greater than 0) were gated as interacting and those with a negative value (less than 0) were gated as not interacting. Features used in the classifier consisted of aspect ratio Brightfield (BF), circularity (BF), major axis (BF), and symmetry BF.

To validate that the classifier was accurate, a template of the training sample analysis was applied to untrained files. All samples were batch-analyzed and a statistics report was generated.

Unsupervised t-SNE and FlowSOM Analysis in OMIQ

.fsc files were exported from Cytek Aurora (Cytek Biosciences) and loaded into OMIQ (Dotmatics) for downstream analysis. The t-distributed stochastic neighbor embedding (t-SNE) analysis was run on equal numbers of events per sample. To visualize each cell on a two-dimensional map, the t-SNE algorithm was applied with 1,000 total iterations with 250 as early exaggeration, perplexity of 30, theta of 0.5, and a learning rate of 5,000. Cells were then clustered by applying the FlowSOM algorithm using the following settings: 12 metaclusters and a self-organizing map grid size of 12×12. All markers in Supplemental Table 12 were used for running the t-SNE and FlowSOM, except the live/dead as dead cells were excluded from the analysis and the G4S antibody as this was overlaid after performing the analysis to identify CAR-positive cell populations. FlowSOM metaclusters were overlaid on the t-SNE maps and the metaclusers were annotated by manual inspection of the expression of surface markers within each metacluster. Heatmaps were generated for relevant surface markers to identify expression in the metaclusters (FIG. 97C-L). A clear decrease in cell count was observed in metacluster 18 in the 22-V4 treated PBMCs in comparison to the PBS-treated group (FIG. 97A). This metacluster was identified to be CD20-positive and thus labeled as B-cells (FIG. 97B and FIG. 97C). No difference was observed in the CD3+CD4+ clusters between the CAR-LNP treated group and the non-treated group (FIG. 97D and FIG. 97E). However, a change in the CD3+CD8+ metacluster was observed in the treated group compared to the untreated control group, which may indicate specific target engagement of the αCD8-targeted LNP (FIG. 97F). No changes were observed in the CD159a+ (NK and NKT) and CD14+ (monocytes) metaclusters (FIG. 97G and FIG. 97H). CAR expression was exclusively observed in the CD8-positive population confirming the previous supervised analysis (FIG. 97I). A slight increase in the expression of the early T-cell activation marker, CD69, was observed in the CD3+CD8+ metacluster indicating CAR-mediated target engagement and activation (FIG. 97L).

SUPPLEMENTAL TABLE 12
Flow Cytometry panel design for PBMC transfection
assay (Table showing the markers and fluorochromes
used for staining human PBMCs. Stained PBMCs were
run on the Cytek Aurora (Cytek Biosciences)).

| Laser | Marker | Dye | Channel | Vendor | Clone |
|---|---|---|---|---|---|
| UV | CD20 | BUV395 | UV2 | BD | 2H7 |
| 355 nm | CD69 | BUV496 | UV7 | BD | FN50 |
| Violet | CD8a | BV421 | V1 | BioLegend | SK1 |
| 405 nm | CD14 | BV650 | V11 | BD | M5E2 |
| | CD95 | BV711 | V13 | BD | DX2 |
| | CD4 | BV786 | V15 | BD | L200 |
| Blue | CCR7 | AF488 | 82 | BioLegend | G043H7 |
| 488 nm | CD4SRA | BB700 | 89 | BD | 5H9 |
| YG | CD159a | PE | YG1 | Beckman | Z199 |
| 561 nm | | | | Coulter | |
| | mCherry | PE-Dz594 | YG3 | — | — |
| Red | G4S | AF647 | R1 | CST | E702V |
| 640 nm | CD3 | AF700 | R4 | BD | SP34-2 |
| | Viability | LD eFluor780 | R7 | Invitrogen | — |

### REFERENCES

1. June, C. H. and M. Sadelain, Chimeric Antigen Receptor Therapy. New England Journal of Medicine, 2018. 379 (1): p. 64-73.
2. June, C. H., et al., CAR T cell immunotherapy for human cancer. Science, 2018. 359 (6382): p. 1361-1365.
3. De Marco, R. C., H. J. Monzo, and P. M. Ojala, CAR T Cell Therapy: A Versatile Living Drug. Int J Mol Sci, 2023. 24 (7).
4. Labanieh, L., R. G. Majzner, and C. L. Mackall, Programming CAR-T cells to kill cancer. Nature Biomedical Engineering, 2018. 2 (6): p. 377-391.
5. Neelapu, S. S., et al., Chimeric antigen receptor T-cell therapy-assessment and management of toxicities. Nature Reviews Clinical Oncology, 2018. 15 (1): p. 47-62.
6. Bonifant, C. L., et al., Toxicity and management in CAR T-cell therapy. Molecular therapy oncolytics, 2016. 3: p. 16011-16011.
7. Bouchkouj, N., et al., FDA Approval Summary: Axicabtagene Ciloleucel for Relapsed or Refractory Large B-cell Lymphoma. Clinical Cancer Research, 2019. 25 (6): p. 1702-1708.
8. Vormittag, P., et al., A guide to manufacturing CAR T cell therapies. Current Opinion in Biotechnology, 2018. 53: p. 164-181.
9. Riley, R. S., et al., Delivery technologies for cancer immunotherapy. Nature Reviews Drug Discovery, 2019. 18 (3): p. 175-196.
10. Steinle, H., et al., Concise Review: Application of In Vitro Transcribed Messenger RNA for Cellular Engineering and Reprogramming: Progress and Challenges. Stem Cells, 2017. 35 (1): p. 68-79.
11. Billingsley, M. M., et al., In Vivo mRNA CAR T Cell Engineering via Targeted Ionizable Lipid Nanoparticles with Extrahepatic Tropism. Small, 2024. 20 (11): p. 2304378.
12. Sahin, U., K. Karikó, and Ö. Türeci, mRNA-based therapeutics—developing a new class of drugs. Nat Rev Drug Discov, 2014. 13 (10): p. 759−80.
13. Hou, X., et al., Lipid nanoparticles for mRNA delivery. Nature Reviews Materials, 2021. 6 (12): p. 1078-1094.
14. Hajj, K. A. and K. A. Whitehead, Tools for translation: non-viral materials for therapeutic mRNA delivery. Nature Reviews Materials, 2017. 2 (10): p. 17056.
15. Guan, S. and J. Rosenecker, Nanotechnologies in delivery of mRNA therapeutics using nonviral vector-based delivery systems. Gene Therapy, 2017. 24 (3): p. 133-143.
16. Kowalski, P. S., et al., Delivering the Messenger: Advances in Technologies for Therapeutic mRNA Delivery. Molecular Therapy, 2019. 27 (4): p. 710-728.
17. Baden, L. R., et al., Efficacy and Safety of the mRNA-1273 SARS-COV-2 Vaccine. New England Journal of Medicine, 2021. 384 (5): p. 403-416.
18. Anderson, E. J., et al., Safety and Immunogenicity of SARS-COV-2 mRNA-1273 Vaccine in Older Adults. New England Journal of Medicine, 2020. 383 (25): p. 2427-2438.
19. Rosenblum, D., et al., Progress and challenges towards targeted delivery of cancer therapeutics. Nature Communications, 2018. 9 (1).
20. Blanco, E., H. Shen, and M. Ferrari, Principles of nanoparticle design for overcoming biological barriers to drug delivery. Nature Biotechnology, 2015. 33 (9): p. 941-951.
21. Hayes, M. E., et al., Increased target specificity of anti-HER2 genospheres by modification of surface charge and degree of PEGylation. Mol Pharm, 2006. 3 (6): p. 726-36.
22. Parayath, N. N., et al., In vitro-transcribed antigen receptor mRNA nanocarriers for transient expression in circulating T cells in vivo. Nature Communications, 2020. 11 (1).
23. Smith, T. T., et al., In situ programming of leukaemia-specific T cells using synthetic DNA nanocarriers. Nature Nanotechnology, 2017. 12 (8): p. 813-820.
24. Zhang, F., et al., Genetic programming of macrophages to perform anti-tumor functions using targeted mRNA nanocarriers. Nature Communications, 2019. 10 (1).
25. Rurik, J. G., et al., CAR T cells produced in vivo to treat cardiac injury. Science, 2022. 375 (6576): p. 91-96.
26. Tombácz, I., et al., Highly efficient CD4+ T cell targeting and genetic recombination using engineered CD4+ cell-homing mRNA-LNPs. Molecular Therapy, 2021. 29 (11): p. 3293-3304.
27. Akinc, A., et al., Targeted delivery of RNAi therapeutics with endogenous and exogenous ligand-based mechanisms. Mol Ther, 2010. 18 (7): p. 1357-64.
28. Veiga, N., et al., Cell specific delivery of modified mRNA expressing therapeutic proteins to leukocytes. Nature Communications, 2018. 9 (1).
29. Ramishetti, S., et al., Systemic Gene Silencing in Primary T Lymphocytes Using Targeted Lipid Nanoparticles. ACS Nano, 2015. 9 (7): p. 6706-6716.
30. Parhiz, H., et al., PECAM-1 directed re-targeting of exogenous mRNA providing two orders of magnitude enhancement of vascular delivery and expression in lungs independent of apolipoprotein E-mediated uptake. J Control Release, 2018. 291: p. 106-115.
31. Kim, M., et al., Engineered ionizable lipid nanoparticles for targeted delivery of RNA therapeutics into different types of cells in the liver. Sci Adv, 2021. 7 (9).
32. Katakowski, J. A., et al., Delivery of siRNAs to Dendritic Cells Using DEC205-Targeted Lipid Nanoparticles to Inhibit Immune Responses. Mol Ther, 2016. 24 (1): p. 146-55.
33. Rosenblum, D., et al., CRISPR-Cas9 genome editing using targeted lipid nanoparticles for cancer therapy. Sci Adv, 2020. 6 (47).
34. Karikó, K., et al., Generating the optimal mRNA for therapy: HPLC purification eliminates immune activation and improves translation of nucleoside-modified, protein-encoding mRNA. Nucleic Acids Research, 2011. 39 (21): p. e142-e142.
35. Geddie, M. L., et al., Development of disulfide-stabilized Fabs for targeting of antibody-directed nanotherapeutics. MAbs, 2022. 14 (1): p. 2083466.
36. Higbee, R. G., et al., An Immunologic Model for Rapid Vaccine Assessment—A Clinical Trial in a Test Tube. Alternatives to Laboratory Animals, 2009. 37 (1_suppl): p. 19-27.
37. Dhir, V., et al., A predictive biomimetic model of cytokine release induced by TGN1412 and other therapeutic monoclonal antibodies. Journal of Immunotoxicology, 2012. 9 (1): p. 34-42.
38. Bonnevaux, H., et al., Pre-clinical development of a novel CD3-CD123 bispecific T-cell engager using cross-over dual-variable domain (CODV) format for acute myeloid leukemia (AML) treatment. Oncoimmunology, 2021. 10 (1): p. 1945803.
39. Kochenderfer, J. N., et al., Construction and Preclinical Evaluation of an Anti-CD19 Chimeric Antigen Receptor. Journal of Immunotherapy, 2009. 32 (7): p. 689-702.
40. Haso, W., et al., Anti-CD22-chimeric antigen receptors targeting B-cell precursor acute lymphoblastic leukemia. Blood, 2013. 121 (7): p. 1165-1174.
41. Mu, X. and S. Hur, Immunogenicity of In Vitro-Transcribed RNA. Acc Chem Res, 2021. 54 (21): p. 4012-4023.
42. Lee, D. W., et al., T cells expressing CD19 chimeric antigen receptors for acute lymphoblastic leukaemia in children and young adults: a phase 1 dose-escalation trial. The Lancet, 2015. 385 (9967): p. 517-528.
43. Kochenderfer, J. N., et al., B-cell depletion and remissions of malignancy along with cytokine-associated toxicity in a clinical trial of anti-CD19 chimeric-antigen-receptor-transduced T cells. Blood, 2012. 119 (12): p. 2709-2720.
44. Gajra, A., et al., Barriers to Chimeric Antigen Receptor T-Cell (CAR-T) Therapies in Clinical Practice. Pharmaceutical Medicine, 2022. 36 (3): p. 163-171.
45. Paunovska, K., D. Loughrey, and J. E. Dahlman, Drug delivery systems for RNA therapeutics. Nature Reviews Genetics, 2022. 23 (5): p. 265-280.
46. Nayerossadat, N., T. Maedeh, and P. A. Ali, Viral and nonviral delivery systems for gene delivery. Adv Biomed Res, 2012. 1: p. 27.
47. Zielińska, A., et al., Polymeric Nanoparticles: Production, Characterization, Toxicology and Ecotoxicology. Molecules, 2020. 25 (16).
48. Sharma, S., R. Parveen, and B. P. Chatterji, Toxicology of Nanoparticles in Drug Delivery. Curr Pathobiol Rep, 2021. 9 (4): p. 133-144.
49. Neelapu, S. S., et al., Axicabtagene Ciloleucel CAR T-Cell Therapy in Refractory Large B-Cell Lymphoma. New England Journal of Medicine, 2017. 377 (26): p. 2531-2544.
50. Schuster, S. J., et al., Chimeric Antigen Receptor T Cells in Refractory B-Cell Lymphomas. New England Journal of Medicine, 2017. 377 (26): p. 2545-2554.
51. Maude, S. L., et al., Tisagenlecleucel in Children and Young Adults with B-Cell Lymphoblastic Leukemia. New England Journal of Medicine, 2018. 378 (5): p. 439-448.
52. Mckinlay, C. J., et al., Enhanced mRNA delivery into lymphocytes enabled by lipid-varied libraries of charge-altering releasable transporters. Proceedings of the National Academy of Sciences, 2018. 115 (26): p. E5859-E5866.

DESCRIPTION OF SEQUENCES

| SEQ | | |
|---|---|---|
| ID NO | Name | Description |
| 1 | A044300805 | T0347015C01(L11V, A14P, D30E, G55E, N56Q, I60A, S71R, K83R, A89L, N100kG, M100oL, |
| | | K105Q) |
| 2 | A044300805_v1 | T0347015C01(L11V, A14P, D30E, G55E, N56Q, I60A, S71R, K83R, A89L, K100bE, N100kG, |
| | | M100oL, K105Q) |
| 3 | A044300805_v2 | T0347015C01(L11V, A14P, D30E, G55E, N56Q, I60A, S71R, K83R, A89L, K100bY, N100kG, |
| | | M100oL, K105Q) |
| 4 | A044300805_v3 | T0347015C01(L11V, A14P, D30E, G55E, N56Q, I60A, S71R, Y79D, K83R, A89L, N100kG, |
| | | M100oL, K105Q) |
| 5 | A044300805_v4 | T0347015C01(L11V, A14P, D30E, G55E, NS6Q, I60A, S71R, Y79D, K83R, A89L, K100bE, |
| | | N100kG, M100oL, K105Q) |
| 6 | A044300805_v5 | T0347015C01(L11V, A14P, D30E, G55E, N56Q, I60A, S71R, Y79D, K83R, A89L, K100bY, |
| | | N100kG, M100oL, K105Q) |
| 7 | A044300805_v6 | T0347015C01(L11V, A14P, D30E, G55E, N56Q, I60A, S71R, Y79S, K83R, A89L, N100kG, |
| | | M100oL, K105Q) |
| 8 | A044300805_v7 | T0347015C01(L11V, A14P, D30E, G55E, N56Q, I60A, S71R, Y79S, K83R, A89L, K100bE, |
| | | N100kG, M100oL, K105Q) |
| 9 | A044300805_v8 | T0347015C01(L11V, A14P, D30E, G55E, N56Q, I60A, S71R, Y79S, K83R, A89L, K100bY, |
| | | N100kG, M100oL, K105Q) |
| 10 | A044300694 | CD8aBDSn(L11V, A14P, N53E, A68T, T69I, K83R, V89L, N101R, Q108L) |
| 11 | A044300694_v1 | CD8aBDSn(L11V, A14P, N53E, A68T, T69I, K83R, V89L, A94E, N101R, Q108L) |
| 12 | A044300694_v2 | CD8aBDSn(L11V, A14P, N53E, A68T, T69I, K83R, V89L, A94Y, N101R, Q108L) |
| 13 | A044300694_v3 | CD8aBDSn(L11V, A14P, N53E, A68T, T69I, K83R, V89L, V100aL, N101R, Q108L) |
| 14 | A044300694_v4 | CD8aBDSn(L11V, A14P, N53E, A68T, T69I, K83R, V89L, A94E, V100aL, N101R, Q108L) |
| 15 | A044300694_v5 | CD8aBDSn(L11V, A14P, N53E, A68T, T69I, K83R, V89L, A94Y, V100aL, N101R, Q108L) |
| 16 | A044300694_v6 | CD8aBDSn(L11V, A14P, N53E, A68T, T69I, Y79D, K83R, V89L, N101R, Q108L) |
| 17 | A044300694_v7 | CD8aBDSn(L11V, A14P, N53E, A68T, T69I, Y79D, K83R, V89L, V100aL, N101R, Q108L) |
| 18 | A044300694_v8 | CD8aBDSn(L11V, A14P, N53E, A6ST, T69I, Y79D, K83R, V89L, A94E, V100aL, N101R, |
| | | Q108L) |
| 19 | A044300694_v9 | CD8aBDSn(L11V, A14P, N53E, A68T, T69I, Y79D, K83R, V89L, A94Y, V100aL, N101R, |
| | | Q108L) |
| 20 | A044300694_v10 | CD8aBDSn(L11V, A14P, N53E, A68T, T69I, Y79D, K83R, V89L, A94E, N101R, Q108L) |
| 21 | A044300694_v11 | CD8aBDSn(L11V, A14P, N53E, A68T, T69I, Y79D, K83R, V89L, A94Y, N101R, Q108L) |
| 22 | A044300694_v12 | CD8aBDSn(L11V, A14P, N53E, A68T, T69I, Y79S, K83R, V89L, V100aL, N101R, Q108L) |
| 23 | A044300694_v13 | CD8aBDSn(L11V, A14P, N53E, A68T, T69I, Y79S, K83R, V89L, A94E, V100aL, N101R, |
| | | Q108L) |
| 24 | A044300694_v14 | CD8aBDSn(L11V, A14P, N53E, A68T, T69I, Y79S, K83R, V89L, A94Y, V100aL, N101R, |
| | | Q108L) |
| 25 | A044300694_v15 | CD8aBDSn(L11V, A14P, N53E, A68T, T69I, Y79S, K83R, V89L, N101R, Q108L) |
| 26 | A044300694_v16 | CD8aBDSn(L11V, A14P, N53E, A68T, T69I, Y79S, K83R, V89L, A94E, N101R, Q108L) |
| 27 | A044300694_v17 | CD8aBDSn(L11V, A14P, N53E, A68T, T69I, Y79S, K83R, V89L, A94Y, N101R, Q108L) |
| 28 | T0347015C01_GGC | T0347015C01-GGC |
| 29 | A044300805_GGC | T0347015C01(L11V, A14P, D30E, G55E, N56Q, I60A, S71R, K83R, A89L, N100kG, |
| | | M100oL, K105Q)_GCC |
| 30 | A044300805_v1_GGC | T0347015C01(L11V, A14P, D30E, G55E, N56Q, I60A, S71R, K83R, A89L, K100bE, |
| | | N100kG, M1000L, K105Q)_GCC |
| 31 | A044300805_v2_GGC | T0347015C01(L11V, A14P, D30E, G55E, N56Q, I60A, S71R, K83R, A89L, K100bY, |
| | | N100kG, M100oL, K105Q)_GCC |
| 32 | A044300805_v3_GGC | T0347015C01(L11V, A14P, D30E, G55E, N56Q, I60A, S71R, Y79D, K83R, A89L, N100kG, |
| | | M1000L, K105Q)_GCC |
| 33 | A044300805_v4_GGC | T0347015C01(L11V, A14P, D30E, G55E, N56Q, I60A, S71R, Y79D, K83R, A89L, K100bE, |
| | | N100kG, M100oL, K105Q)_GCC |
| 34 | A044300805_v5_GGC | T0347015C01(L11V, A14P, D30E, G55E, N56Q, I60A, S71R, Y79D, K83R, A89L, K100bY, |
| | | N100kG, M100oL, K105Q)_GCC |
| 35 | A044300805_v6_GGC | T0347015C01(L11V, A14P, D30E, G55E, N56Q, I60A, S71R, Y79S, K83R, A89L, N100kG, |
| | | M100oL, K105Q)_GCC |
| 36 | A044300805_v7_GGC | T034701SC01(L11V, A14P, D30E, G55E, N56Q, I60A, S71R, Y79S, K83R, A89L, K100bE, |
| | | N100kG, M100oL, K105Q)_GCC |
| 37 | VHH6 | anti-HuCD7 VHH6 VHH-CD8a |
| 38 | TRX2 bDS (HC) | anti-hCD8 TRX2 Fab bDS (HO) |
| 39 | TRX2 bDS (LC) | anti-hCD8 TRX2 Fab bDS (LC) |
| 40 | hSP34 bDS (HC) | anti-HuCD3 hSP34-hlam bDS (HO) |
| 41 | hSP34 bDS (LC) | anti-HuCD3 hSP34-hlam bDS (LC) |
| 42 | mutOKT8 NoDS (HC) | anti-CD8 mutOKT8 Fab NoDs (HC) |
| 43 | mutOKT8 NoDS (LC) | anti-CD8 mutOKT8 Fab NoDs (LC) |
| 44 | A044300805_v8_GGC | T0347015C01(L11V, A14P, D30E, G55E, N56Q, I60A, S71R, Y79S, K83R, A89L, K100bY, |
| | | N100kG, M100oL, K105Q)_GCC |
| 45 | PolyA | Poly A in CAR mRNA |
| 46 | P49 with CD28 hinge | 49-22-28-40-28-3z |
| 47 | P49 with short CDS hinge | 49-22-8-26-28-3z |
| 48 | P49 with intermediate CD8 hinge | 49-22-8-46-28-3z |
| 49 | P49 with long CD8 hinge | 49-22-8-66-28-3z |
| 50 | P155 with CD28 hinge | 155-22-28-40-28-3z |
| 51 | P155 with short CD8 hinge | 155-22-8-26-28-3z |
| 52 | P155 with intermediate CD8 hinge | 155-22-8-46-28-3z |
| 53 | P155 with long CD8 hinge | 155-22-8-66-28-3z |
| 54 | P197 with CD28 hinge | 197-22-28-40-28-3z |
| 55 | P197 with short CD8 hinge | 197-22-8-26-28-3z |
| 56 | P197 with intermediate CD8 hinge | 197-22-8-46-28-3z |
| 57 | P197 with long CD8 hinge | 197-22-8-66-28-3z |
| 58 | P198 with CD28 hinge | 198-22-28-40-28-3z |
| 59 | P198 with short CD8 hinge | 198-22-8-26-28-3z |
| 60 | P198 with intermediate CD& hinge | 198-22-8-46-28-3z |
| 61 | P198 with long CD8 hinge | 198-22-8-66-28-3z |
| 62 | m971 with CD28 hinge | m971-22-28-40-28-3z |
| 63 | m971 with short CD8 hinge | m971-8-26-28-3z |
| 64 | m971 with intermediate CD8 hinge | m971-8-46-28-3z |
| 55 | m971 with long CD8 hinge | m971-8-66-28-3z |
| 66 | P49 with CD28 hinge | 49-22-28-40-28-3z |
| 67 | P49 with short CD8 hinge | 49-22-8-26-28-37 |
| 68 | P49 with intermediate CD8 hinge | 49-22-8-46-28-3z |
| 69 | P49 with long CD8 hinge | 49-22-8-66-28-3z |
| 70 | P155 with CD28 hinge | 155-22-28-40-28-3z |
| 71 | P155 with short CD8 hinge | 155-22-8-26-28-3z |
| 72 | P155 with intermediate CD8 hinge | 155-22-8-46-28-3z |
| 73 | P155 with long CD8 hinge | 155-22-8-66-28-3z |
| 74 | P197 with CD28 hinge | 197-22-28-40-28-3z |
| 75 | P197 with short CD8 hinge | 197-22-8-26-28-3z |
| 76 | P197 with intermediate CD8 hinge | 197-22-8-46-28-3z |
| 77 | P197 with long CD8 hinge | 197-22-8-66-28-3z |
| 78 | P198 with CD28 hinge | 198-22-28-40-28-3z |
| 79 | P198 with short CD8 hinge | 198-22-8-26-28-3z |
| 80 | P198 with intermediate CD8 hinge | 198-22-8-46-28-3z |
| 81 | P198 with long CD8 hinge | 198-22-8-66-28-3z |
| 82 | m971 with CD28 hinge | m971-22-28-40-28-3z |
| 83 | m971 with short CD8 hinge | m971-8-26-28-3z |
| 84 | m971 with intermediate CD8 hinge | m971-8-46-28-3z |
| 85 | m971 with long CD8 hinge | m971-8-66-28-3z |
| 86 | TTR-034 | mkap-CD22-P49-CD28-40 |
| 87 | TTR-035 | mkap-CD22-P155-CD28-40 |
| 88 | TTR-036 | mkap-CD22-P155-CD8-46 |
| 89 | TTR-039 | mkap-CD22-P197-CD8-26 |
| 90 | TTR-040 | mkap-CD22-P197-CD8-46 |
| 91 | TTR-041 | CD8a-CD22-P197-CD8-46 |
| 92 | TTR-042 | mkap-CD22-P198-CD28-40 |
| 93 | TTR-043 | mkap-CD22-M2-CD28-40 |
| 94 | TTR-044 | mkap-CD22-m971-CD8-46 |
| 95 | TTR-045 | mkap-CD22-m971-CD8-66 |
| 96 | TTR-046 | mkap-CD22-m971DB-CD8-46 |
| 97 | TTR-034 | mkap-CD22-P49-CD28-40 |
| 98 | TTR-035 | mkap-CD22-P155-CD28-40 |
| 99 | TTR-036 | mkap-CD22-P155-CD8-46 |
| 100 | TTR-039 | mkap-CD22-P197-CD8-26 |
| 101 | TTR-040 | mkap-CD22-P197-CD8-46 |
| 102 | TTR-041 | CD8a-CD22-P197-CD8-46 |
| 103 | TTR-042 | mkap-CD22-P198-CD28-40 |
| 104 | TTR-043 | mkap-CD22-M2-CD28-40 |
| 105 | TTR-044 | mkap-CD22-m971-CD8-46 |
| 106 | TTR-045 | mkap-CD22-m971-CD8-66 |
| 107 | TTR-046 | mkap-CD22-m971DB-CD8-46 |
| 108 | TTR-102 | CD8a-CD22-P49-CD28-40 |
| 109 | TTR-103 | CD8a-CD22-P155-CD28-40 |
| 110 | TTR-105 | CD8a-CD22-P197-CD8-26 |
| 111 | TTR-106 | CD8a-CD22-P198-CD28-40 |
| 112 | TTR-107 | CD8a-CD22-M2-CD28-40 |
| 113 | TTR-108 | CD8a-CD22-m971DB-CD8-46 |
| 114 | TTR-110 | CD8a-CD19-FMC63-CD28-40 |
| 115 | TTR-121 | CD8a-CD22-P49-CD28-40-CD137 |
| 116 | TTR-102 | CD8a-CD22-P49-CD28-40 |
| 117 | TTR-103 | CD8a-CD22-P155-CD28-40 |
| 118 | TTR-105 | CD8a-CD22-P197-CD8-26 |
| 119 | TTR-106 | CD8a-CD22-P198-CD28-40 |
| 120 | TTR-107 | CD8a-CD22-M2-CD28-40 |
| 121 | TTR-108 | CD8a-CD22-m971DB-CD8-46 |
| 122 | TTR-110 | CD8a-CD19-FMC63-CD28-40 |
| 123 | TTR-121 | CD8a-CD22-P49-CD28-40-CD137 |
| 124 | MART14577 | CD8a-CD22-P49-CD28-40 (CoE UTRs) |
| 125 | MRT14580 | CD8a-CD22-P49-CD28-40-CD137 (CoE UTRs) |
| 126 | MRT14577 | CD8a-CD22-P49-CD28-40 (CoE UTRs) |
| 127 | MRT14580 | CD8a-CD22-P49-CD28-40-CD137 (CoE UTRs) |
| 128 | TTR-034 | mkap-CD22-P49-CD28-40 |
| 129 | TTR-035 | mkap-CD22-P155-CD28-40 |
| 130 | TTR-036 | mkap-CD22-P155-CD8-46 |
| 131 | TTR-039 | mkap-CD22-P197-CD8-26 |
| 132 | TTR-040 | mkap-CD22-P197-CD8-46 |
| 133 | TTR-041 | CD8a-CD22-P197-CD8-46 |
| 134 | TTR-042 | mkap-CD22-P198-CD28-40 |
| 135 | TTR-043 | mkap-CD22-M2-CD28-40 |
| 136 | TTR-044 | mkap-CD22-m971-CD8-46 |
| 137 | TTR-045 | mkap-CD22-m971-CD8-66 |
| 138 | TTR-046 | mkap-CD22-m971DB-CD8-46 |
| 139 | TTR-102 | CD8a-CD22-P49-CD28-40 |
| 140 | TTR-103 | CD8a-CD22-P155-CD28-40 |
| 141 | TTR-105 | CD8a-CD22-P197-CD8-26 |
| 142 | TTR-106 | CD8a-CD22-P198-CD28-40 |
| 143 | TTR-107 | CD8a-CD22-M2-CD28-40 |
| 144 | TTR-108 | CD8a-CD22-m971DB-CD8-46 |
| 145 | TTR-110 | CD8a-CD19-FMC63-CD28-40 |
| 146 | TTR-121 | CD8a-CD22-P49-CD28-40-CD137 |
| 147 | MRT14577 | CD8a-CD22-P49-CD28-40 (CoE UTRs) |
| 148 | IMRT14580 | CD8a-CD22-P49-CD28-40-CD137 (CoE UTRs) |
| 149 | Human CD8a-Fc-HIS6 | |
| 150 | Human CD8a | |
| 151 | Fc | |
| 152 | HIS6 | |
| 153 | Human CD8h-Fc-Twinstrep | |
| 154 | Twinstrep | |
| 155 | Cynomolgous CD8a-Fc-HIS6 | |
| 156 | Cynomolgous CD8a | |
| 157 | Cynomolgous CD8b-Fc-Twinstrep | |
| 158 | Cynomolgous CD8b | |
| 159 | TTR-023 | mkap-CD20-Leu16-CD28-40 |
| 160 | T0347015C01 (Abm CDR) | T0347015C01 |
| 161 | A044300805 (Abm CDR) | T0347015C01(L11V, A14P, D30E, G55E, N56Q, I60A, S71R, K83R, A89L, N100kG, |
| | | M100oL, K105Q) |
| 162 | A044300805_v1 (Abm CDR) | T0347015C01(L11V, A14P, D30E, G55E, N56Q, I60A, S71R, K83R, A89L, K100bE, |
| | | N100kG, M100oL, K105Q) |
| 163 | A044300805_v2 (Abm CDR) | T0347015C01(L11V, A14P, D30E, G55E, N56Q, I60A, S71R, K83R, A89L, K100bY, |
| | | N100kG, M100oL, K105Q) |
| 164 | A044300805_v3 (Abm CDR) | T0347015C01(L11V, A14P, D30E, G55E, N56Q, I60A, S71R, Y79D, K83R, A89L, |
| | | N100kG, M100oL, K105Q) |
| 165 | A044300805_v4 (Abm CDR) | T0347015C01(L11V, A14P, D30E, G55E, N56Q, I60A, S71R, Y79D, K83R, A89L, |
| | | K100bE, N100kG, M100oL, K105Q) |
| 166 | A044300805_v5 (Abm CDR) | 10347015C01(L11V, A14P, D30E, G55E, N56Q, I60A, S71R, Y79D, K83R, A89L, |
| | | K100bY, N100kG, M100oL, K105Q) |
| 167 | A044300805_v6 (Abm CDR) | T0347015C01(L11V, A14P, D30E, G55E, NS6Q, I60A, S71R, Y79S, K83R, A89L, N100kG, |
| | | M100oL, K105Q) |
| 168 | A044300805_v7 (Abm CDR) | T0347015C01(L11V, A14P, D30E, G55E, N56Q, I60A, S71R, Y795, K83R, A89L, K100bE, |
| | | N100kG, M100oL, K105Q) |
| 169 | A044300805_v8 (Abm CDR) | T0347015C01(L11V, A14P, D30E, G55E, N56Q, I60A, S71R, Y795, K83R, A89L, K100bY, |
| | | N100kG, M100oL, K105Q) |
| 170 | T0347015C01 (Kabat CDR) | T0347015C01 |
| 171 | A044300805 (Kabat CDR) | T0347015C01(L11V, A14P, D30E, G55E, N56Q, I60A, S71R, K83R, A89L, N100kG, M100oL, |
| | | K105Q) |
| 172 | A044300805_v1 (Kabat CDR) | T0347015C01(L11V, A14P, D30E, G55E, N56Q, I60A, S71R, K83R, A89L, K100bE, N100kG, |
| | | M100oL, K105Q) |
| 173 | A044300805_v2 (Kabat CDR) | T0347015C01(L11V, A14P, D30E, G55E, N56Q, I60A, S71R, K83R, A89L, K100bY, N100kG, |
| | | M100oL, K105Q) |
| 174 | A044300805_v3 (Kabat CDR) | T0347015C01(L11V, A14P, D30E, G55E, N56Q, I60A, S71R, Y79D, K83R, A89L, N100kG, |
| | | M100oL, K105Q) |
| 175 | A044300805_v4 (Kabat CDR) | T0347015C01(LIIV, A14P, D30E, G55E, N56Q, I60A, S71R, Y79D, K83R, A89L, K100bE, |
| | | N100kG, M100oL, K105Q) |
| 176 | A044300805_v5 (Kabat CDR) | T0347015C01(L11V, A14P, D30E, G55E, N56Q, I60A, S71R, Y79D, K83R, A89L, K100bY, |
| | | N100kG, M100oL, K105Q) |
| 177 | A044300805_v6 (Kabat CDR) | T0347015C01(L11V, A14P, D30E, G55E, N56Q, I60A, S71R, Y79S, K83R, A89L, N100kG, |
| | | M100oL, K105Q) |
| 178 | A044300805_v7 (Kabat CDR) | T0347015C01(L11V, A14P, D30E, G55E, N56Q, I60A, S71R, Y795, K83R, A89L, K100bE, |
| | | N100kG, M100oL, K105Q) |
| 179 | A044300805_v8 (Kabat CDR) | T0347015C01(L11V, A14P, D30E, G55E, N56Q, I60A, S71R, Y79S, K83R, A89L, K100bY, |
| | | N100kG, M100oL, K105Q) |
| 180 | T0347015C01 (Abm CDR)_fw1 | fw1 |
| 187 | A044300805 (Abm CDR)_fw1 | fw1 |
| 194 | A044300805_v1 (Abm CDR)_fw1 | fw1 |
| 201 | A044300805_v2 (Abm CDR)_fw1 | fw1 |
| 208 | A044300805_v3 (Abm CDR)_fw1 | fw1 |
| 215 | A044300805_v4 (Abm CDR)_fw1 | fw1 |
| 222 | A044300805_v5 (Abm CDR)_fw1 | fw1 |
| 229 | A044300805_v6 (Abm CDR)_fw1 | fw1 |
| 236 | A044300805_v7 (Abm CDR)_fw1 | fw1 |
| 243 | A044300805_v8 (Abm CDR)_fw1 | fw1 |
| 181 | T0347015C01 (Abm CDR)_CDR1 | CDR1 |
| 188 | A044300805 (Abm CDR)_CDR1 | CDR1 |
| 195 | A044300805_v1 (Abm CDR)_CDR1 | CDR1 |
| 202 | A044300805_v2 (Abm CDR)_CDR1 | CDR1 |
| 209 | A044300805_v3 (Abm CDR)_CDR1 | CDR1 |
| 216 | A044300805_v3 (Abm CDR)_CDR1 | CDR1 |
| 223 | A044300805_v3 (Abm CDR)_CDR1 | CDR1 |
| 230 | A044300805_v3 (Abm CDR)_CDR1 | CDR1 |
| 237 | A044300805_v3 (Abm CDR)_CDR1 | CDR1 |
| 244 | A044300805_v3 (Abm CDR)_CDR1 | CDR1 |
| 182 | T0347015C01 (Abm CDR)_fw2 | fw2 |
| 189 | A044300805 (Abm CDR)_fw2 | fw2 |
| 196 | A044300805_v1 (Abm CDR)_fw2 | fw2 |
| 203 | A044300805_v2 (Abm CDR)_fw2 | fw2 |
| 210 | A044300805_v3 (Abm CDR)_fw2 | fw2 |
| 217 | A044300805_v4 (Abm CDR)_fw2 | fw2 |
| 224 | A044300805_v5 (Abm CDR)_fw2 | fw2 |
| 231 | A044300805_v6 (Abm CDR)_fw2 | fw2 |
| 238 | A044300805_v7 (Abm CDR)_fw2 | fw2 |
| 245 | A044300805_v8 (Abm CDR)_fw2 | fw2 |
| 183 | T0347015C01 (Abm CDR)_CDR2 | CDR2 |
| 190 | A044300805 (Abm CDR)_CDR2 | CDR2 |
| 197 | A044300805_v1 (Abm CDR)_CDR2 | CDR2 |
| 204 | A044300805_v2 (Abm CDR)_CDR2 | CDR2 |
| 211 | A044300805_v3 (Abm CDR)_CDR2 | CDR2 |
| 218 | A044300805_v3 (Abm CDR)_CDR2 | CDR2 |
| 225 | A044300805_v3 (Abm CDR)_CDR2 | CDR2 |
| 232 | A044300805_v3 (Abm CDR)_CDR2 | CDR2 |
| 239 | A044300805_v3 (Abm CDR)_CDR2 | CDR2 |
| 246 | A044300805_v3 (Abm CDR)_CDR2 | CDR2 |
| 184 | T0347015C01 (Abm CDR)_fw3 | fw3 |
| 191 | A044300805 (Abm CDR)_fw3 | fw3 |
| 198 | A044300805_v1 (Abm CDR)_fw3 | fw3 |
| 205 | A044300805_v2 (Abm CDR)_fw3 | fw3 |
| 212 | A044300805_v3 (Abm CDR)_fw3 | fw3 |
| 219 | A044300805_v4 (Abm CDR)_fw3 | fw3 |
| 226 | A044300805_v5 (Abm CDR)_fw3 | fw3 |
| 233 | A044300805_v6 (Abm CDR)_fw3 | fw3 |
| 240 | A044300805_v7 (Abm CDR)_fw3 | fw3 |
| 247 | A044300805_v8 (Abm CDR)_fw3 | fw3 |
| 185 | T0347015C01 (Abm CDR)_CDR3 | CDR3 |
| 192 | A044300805 (Abm CDR)_CDR3 | CDR3 |
| 199 | A044300805_v1 (Abm CDR)_CDR3 | CDR3 |
| 206 | A044300805_v2 (Abm CDR)_CDR3 | CDR3 |
| 213 | A044300805_v3 (Abm CDR)_CDR3 | CDR3 |
| 220 | A044300805_v3 (Abm CDR)_CDR3 | CDR3 |
| 227 | A044300805_v3 (Abm CDR)_CDR3 | CDR3 |
| 234 | A044300805_v3 (Abm CDR)_CDR3 | CDR3 |
| 241 | A044300805_v3 (Abm CDR)_CDR3 | CDR3 |
| 248 | A044300805_v3 (Abm CDR)_CDR3 | CDR3 |
| 186 | T0347015C01 (Abm CDR)_fw4 | fw4 |
| 193 | A044300805 (Abm CDR)_fw4 | fw4 |
| 200 | A044300805_v1 (Abm CDR)_fw4 | fw4 |
| 207 | A044300805_v2 (Abm CDR)_fw4 | fw4 |
| 214 | A044300805_v3 (Abm CDR)_fw4 | fw4 |
| 221 | A044300805_v4 (Abm CDR)_fw4 | fw4 |
| 228 | A044300805_v5 (Abm CDR)_fw4 | fw4 |
| 235 | A044300805_v6 (Abm CDR)_fw4 | fw4 |
| 242 | A044300805_v7 (Abm CDR)_fw4 | fw4 |
| 249 | A044300805_v8 (Abm CDR)_fw4 | fw4 |
| 250 | T0347015C01 (Kabat CDR)_fw1 | fw1 |
| 257 | A044300805 (Kabat CDR)_fw1 | fw1 |
| 264 | A044300805_v1 (Kabat CDR)_fw1 | fw1 |
| 271 | A044300805_v2 (Kabat CDR)_fw1 | fw1 |
| 278 | A044300805_v3 (Kabat CDR)_fw1 | fw1 |
| 285 | A044300805_v4 (Kabat CDR)_fw1 | fw1 |
| 292 | A044300805_v5 (Kabat CDR)_fw1 | fw1 |
| 299 | A044300805_v6 (Kabat CDR)_fw1 | fw1 |
| 306 | A044300805_v7 (Kabat CDR)_fw1 | fw1 |
| 313 | A044300805_v8 (Kabat CDR)_fw1 | fw1 |
| 251 | T0347015C01 (Kabat CDR)_CDR1 | CDR1 |
| 258 | A044300805 (Kabat CDR)_CDR1 | CDR1 |
| 265 | A044300805_v1 (Kabat CDR)_CDR1 | CDR1 |
| 272 | A044300805_v2 (Kabat CDR)_CDR1 | CDR1 |
| 279 | A044300805_v3 (Kabat CDR)_CDR1 | CDR1 |
| 286 | A044300805_v3 (Kabat CDR)_CDR1 | CDR1 |
| 293 | A044300805_v3 (Kabat CDR)_CDR1 | CDR1 |
| 300 | A044300805_v3 (Kabat CDR)_CDR1 | CDR1 |
| 307 | A044300805_v3 (Kabat CDR)_CDR1 | CDR1 |
| 314 | A044300805_v3 (Kabat CDR)_CDR1 | CDR1 |
| 252 | T0347015C01 (Kabat CDR)_fw2 | fw2 |
| 259 | A044300805 (Kabat CDR)_fw2 | fw2 |
| 266 | A044300805_v1 (Kabat CDR)_fw2 | fw2 |
| 273 | A044300805_v2 (Kabat CDR)_fw2 | fw2 |
| 280 | A044300805_v3 (Kabat CDR)_fw2 | fw2 |
| 287 | A044300805_v4 (Kabat CDR)_fw2 | fw2 |
| 294 | A044300805_v5 (Kabat CDR)_fw2 | fw2 |
| 301 | A044300805_v6 (Kabat CDR)_fw2 | fw2 |
| 308 | A044300805_v7 (Kabat CDR)_fw2 | fw2 |
| 315 | A044300805_v8 (Kabat CDR)_fw2 | fw2 |
| 253 | T0347015C01 (Kabat CDR)_CDR2 | CDR2 |
| 260 | A044300805 (Kabat CDR)_CDR2 | CDR2 |
| 267 | A044300805_v1 (Kabat CDR)_CDR2 | CDR2 |
| 274 | A044300805_v2 (Kabat CDR)_CDR2 | CDR2 |
| 281 | A044300805_v3 (Kabat CDR)_CDR2 | CDR2 |
| 288 | A044300805_v3 (Kabat CDR)_CDR2 | CDR2 |
| 295 | A044300805_v3 (Kabat CDR)_CDR2 | CDR2 |
| 302 | A044300805_v3 (Kabat CDR)_CDR2 | CDR2 |
| 309 | A044300805_v3 (Kabat CDR)_CDR2 | CDR2 |
| 316 | A044300805_v3 (Kabat CDR)_CDR2 | CDR2 |
| 254 | T0347015C01 (Kabat CDR)_fw3 | fw3 |
| 261 | A044300805 (Kabat CDR)_fw3 | fw3 |
| 268 | A044300805_v1 (Kabat CDR)_fw3 | fw3 |
| 275 | A044300805_v2 (Kabat CDR)_fw3 | fw3 |
| 282 | A044300805_v3 (Kabat CDR)_fw3 | fw3 |
| 289 | A044300805_v4 (Kabat CDR)_fw3 | fw3 |
| 296 | A044300805_v5 (Kabat CDR)_fw3 | fw3 |
| 303 | A044300805_v6 (Kabat CDR)_fw3 | fw3 |
| 310 | A044300805_v7 (Kabat CDR)_fw3 | fw3 |
| 317 | A044300805_v8 (Kabat CDR)_fw3 | fw3 |
| 255 | T0347015C01 (Kabat CDR)_CDR3 | CDR3 |
| 262 | A044300805 (Kabat CDR)_CDR3 | CDR3 |
| 269 | A044300805_v1 (Kabat CDR)_CDR3 | CDR3 |
| 276 | A044300805_v2 (Kabat CDR)_CDR3 | CDR3 |
| 283 | A044300805_v3 (Kabat CDR)_CDR3 | CDR3 |
| 290 | A044300805_v3 (Kabat CDR)_CDR3 | CDR3 |
| 297 | A044300805_v3 (Kabat CDR)_CDR3 | CDR3 |
| 304 | A044300805_v3 (Kabat CDR)_CDR3 | CDR3 |
| 311 | A044300805_v3 (Kabat CDR)_CDR3 | CDR3 |
| 318 | A044300805_v3 (Kabat CDR)_CDR3 | CDR3 |
| 256 | T0347015C01 (Kabat CDR)_fw4 | fw4 |
| 263 | A044300805 (Kabat CDR)_fw4 | fw4 |
| 270 | A044300805_v1 (Kabat CDR)_fw4 | fw4 |
| 277 | A044300805_v2 (Kabat CDR)_fw4 | fw4 |
| 284 | A044300805_v3 (Kabat CDR)_fw4 | fw4 |
| 291 | A044300805_v4 (Kabat CDR)_fw4 | fw4 |
| 298 | A044300805_v5 (Kabat CDR)_fw4 | fw4 |
| 305 | A044300805_v6 (Kabat CDR)_fw4 | fw4 |
| 312 | A044300805_v7 (Kabat CDR)_fw4 | fw4 |
| 319 | A044300805_v8 (Kabat CDR)_fw4 | Tw4 |
| 320 | Alb8 | synthetic construct |
| 321 | Alb23 | synthetic construct |
| 322 | Alb129 | synthetic construct |
| 323 | Alb132 | synthetic construct |
| 324 | Alb11 | synthetic construct |
| 325 | Alb11 (5112K)-A | synthetic construct |
| 326 | Alb82 | synthetic construct |
| 327 | Alb82-A | synthetic construct |
| 328 | Alb82-AA | synthetic construct |
| 329 | Alb82-AAA | synthetic construct |
| 330 | Alb82-G | synthetic construct |
| 331 | Alb82-GG | synthetic construct |
| 332 | Alb82-GGG | synthetic construct |
| 333 | Alb223 | synthetic construct |
| 334 | AlbX00001 | synthetic construct |
| 335 | Alb23002 | synthetic construct |
| 336 | 3A linker | synthetic construct |
| 337 | 5GS linker | synthetic construct |
| 338 | 7GS linker | synthetic construct |
| 339 | 8GS linker | synthetic construct |
| 340 | 9GS linker | synthetic construct |
| 341 | 10GS linker | synthetic construct |
| 342 | 15GS linker | synthetic construct |
| 343 | 18GS linker | synthetic construct |
| 344 | 20GS linker | synthetic construct |
| 345 | 25GS linker | synthetic construct |
| 346 | 30GS linker | synthetic construct |
| 347 | 35GS linker | synthetic construct |
| 348 | 40GS linker | synthetic construct |
| 349 | G1 hinge | synthetic construct |
| 350 | 9GS-G1 hinge | synthetic construct |
| 351 | Llama upper long hinge region | synthetic construct |
| 352 | G3 hinge | synthetic construct |
| 353 | | synthetic construct |
| 354 | | synthetic construct |
| 355 | | synthetic construct |
| 356 | | synthetic construct |
| 357 | | synthetic construct |
| 358 | | synthetic construct |
| 359 | | synthetic construct |
| 360 | | synthetic construct |
| 361 | | synthetic construct |
| 362 | | synthetic construct |
| 363 | | synthetic construct |
| 364 | | synthetic construct |
| 365 | | synthetic construct |
| 366 | | synthetic construct |
| 367 | | synthetic construct |
| 368 | | synthetic construct |
| 369 | | synthetic construct |
| 370 | | synthetic construct |
| 371 | | synthetic construct |
| 372 | | synthetic construct |
| 373 | | synthetic construct |
| 374 | | synthetic construct |
| 375 | | synthetic construct |
| 376 | | synthetic construct |
| 377 | | synthetic construct |
| 378 | | synthetic construct |
| 379 | | synthetic construct |
| 380 | | synthetic construct |
| 381 | | synthetic construct |
| 382 | | synthetic construct |
| 383 | | synthetic construct |
| 384 | | synthetic construct |
| 385 | | synthetic construct |
| 386 | | synthetic construct |
| 387 | | synthetic construct |
| 388 | | synthetic construct |
| 389 | | synthetic construct |
| 390 | | synthetic construct |
| 391 | | synthetic construct |
| 392 | | synthetic construct |
| 393 | | synthetic construct |
| 394 | | synthetic construct |
| 395 | | synthetic construct |
| 396 | | synthetic construct |
| 397 | | synthetic construct |
| 398 | | synthetic construct |
| 399 | | synthetic construct |
| 400 | | synthetic construct |
| 401 | | synthetic construct |
| 402 | | synthetic construct |
| 403 | | synthetic construct |
| 404 | | synthetic construct |
| 405 | | synthetic construct |
| 406 | | synthetic construct |
| 407 | | synthetic construct |
| 408 | | synthetic construct |
| 409 | | synthetic construct |
| 410 | | synthetic construct |
| 411 | | synthetic construct |
| 412 | | synthetic construct |
| 413 | | synthetic construct |
| 414 | | synthetic construct |
| 415 | | synthetic construct |
| 416 | | synthetic construct |
| 417 | | synthetic construct |
| 418 | | synthetic construct |
| 419 | BDSn ISVD | full sequence |
| 420 | BDSn ISVD_fw1 | fw1 |
| 421 | BDSn ISVD_CDR1 | CDR1 |
| 422 | BDSn ISVD_fw2 | fw2 |
| 423 | BDSn ISVD_CDR2 | CDR2 |
| 424 | BDSn ISVD_fw3 | fw3 |
| 425 | BDSn ISVD_CDR3 | CDR3 |
| 426 | BDSn ISVD_fw4 | fw4 |
| 427 | | synthetic construct |
| 428 | | synthetic construct |
| 429 | | synthetic construct |
| 430 | | synthetic construct |
| 431 | | synthetic construct |
| 432 | | synthetic construct |
| 433 | | synthetic construct |
| 434 | | synthetic construct |
| 435 | | synthetic construct |
| 436 | | synthetic construct |
| 437 | | synthetic construct |
| 438 | | synthetic construct |
| 439 | | synthetic construct |
| 440 | | synthetic construct |
| 441 | | synthetic construct |
| 442 | | synthetic construct |
| 443 | | synthetic construct |
| 444 | | synthetic construct |
| 445 | | synthetic construct |
| 446 | | synthetic construct |
| 447 | | synthetic construct |
| 448 | | synthetic construct |
| 449 | | synthetic construct |
| 450 | | synthetic construct |
| 451 | | synthetic construct |
| 452 | | synthetic construct |
| 453 | | synthetic construct |
| 454 | | synthetic construct |
| 455 | | synthetic construct |
| 456 | | synthetic construct |
| 457 | | synthetic construct |
| 458 | | synthetic construct |
| 459 | | synthetic construct |
| 460 | | synthetic construct |
| 461 | | synthetic construct |
| 462 | | synthetic construct |
| 463 | | Mus musculus |
| 464 | | synthetic construct |
| 465 | | synthetic construct |
| 466 | | synthetic construct |
| 467 | | synthetic construct |
| 468 | | synthetic construct |
| 469 | | synthetic construct |
| 470 | | synthetic construct |
| 471 | | Homo sapiens |
| 472 | | synthetic construct |
| 473 | | synthetic construct |
| 474 | | synthetic construct |
| 475 | | synthetic construct |
| 476 | | synthetic construct |
| 477 | | synthetic construct |
| 478 | | synthetic construct |
| 479 | | synthetic construct |
| 480 | | synthetic construct |
| 481 | | synthetic construct |
| 482 | | synthetic construct |
| 483 | | synthetic construct |
| 484 | | synthetic construct |
| 485 | | synthetic construct |
| 486 | | synthetic construct |
| 487 | | synthetic construct |
| 488 | | synthetic construct |
| 489 | | synthetic construct |
| 490 | | synthetic construct |
| 491 | | synthetic construct |
| 492 | | synthetic construct |
| 493 | | synthetic construct |
| 494 | | synthetic construct |
| 495 | | synthetic construct |
| 496 | | synthetic construct |
| 497 | | synthetic construct |
| 498 | | synthetic construct |
| 499 | | synthetic construct |
| 500 | | synthetic construct |
| 501 | | synthetic construct |
| 502 | | synthetic construct |
| 503 | | synthetic construct |
| 504 | | synthetic construct |
| 505 | | synthetic construct |
| 506 | | synthetic construct |
| 507 | | synthetic construct |
| 508 | | synthetic construct |
| 509 | | synthetic construct |
| 510 | | synthetic construct |
| 511 | | synthetic construct |
| 512 | | synthetic construct |
| 513 | | synthetic construct |
| 514 | O49 (Pro L) light chain | synthetic construct |
| 515 | Leading aa before VH | synthetic construct |
| 516 | 5′ UTR (MRT14577) | Synthetic construct |
| 517 | 3′ URT (MRT14577) | synthetic construct |
| 518 | 5′ UTR (TTR-102) | synthetic construct |
| 519 | 3′ UTR (TTR-102) | synthetic construct |
| 520 | Leading aa before VH | synthetic construct |
| 521 | CD8alpha hinge-transmembrane (66) | human |
| 522 | CD28 hinge-transmembrane | human |
| 523 | P49 VH | synthetic construct |
| 524 | P49 VL (ProL) | synthetic construct |
| 525 | P155 VH | synthetic construct |
| 526 | P155 VL | synthetic construct |
| 527 | P197 VH | synthetic construct |
| 528 | P197 VL (ProL) | synthetic construct |
| 529 | P198 VH | synthetic construct |
| 530 | P198 VL | synthetic construct |
| 531 | P12 VH | synthetic construct |
| 532 | P12 VL (ProL) | synthetic construct |
| 533 | P12 VL WT | synthetic construct |
| 534 | P49 VL WT | synthetic construct |
| 535 | P197 VL WT | synthetic construct |
| 536 | m971 VH | synthetic construct |
| 537 | m971 VL | synthetic construct |
| 538 | 46 aa CD8 hinge-transmembrane | human |
| 539 | 26 aa CD8 hinge-transmembrane | human |
| 540 | 66 aa CD8 hinge-transmembrane | human |
| 541 | CD28 Hinge | human |
| 542 | CD28 TM | human |
| 543 | CD28 co-stim | human |
| 544 | CD3 zeta | human |
| 545 | P49 with CD28 hinge | 49-22-28-40-28-3z |
| 546 | P49 with short CD8 hinge | 49-22-8-26-28-3z |
| 547 | P49 with intermediate CD8 hinge | 49-22-8-46-28-3z |
| 548 | P49 with long CD8 hinge | 49-22-8-66-28-3z |
| 549 | P155 with CD28 hinge | 155-22-28-40-28-3z |
| 550 | P155 with short CD8 hinge | 155-22-8-26-28-3z |
| 551 | P155 with intermediate CD8 hinge | 155-22-8-46-28-3z |
| 552 | P155 with long CD8 hinge | 155-22-8-66-28-3z |
| 553 | P197 with CD28 hinge | 197-22-28-40-28-3z |
| 554 | P197 with short CD8 hinge | 197-22-8-26-28-3z |
| 555 | P197 with intermediate CD8 hinge | 197-22-8-46-28-3z |
| 556 | P197 with long CD8 hinge | 197-22-8-66-28-3z |
| 557 | P198 with CD28 hinge | 198-22-28-40-28-3z |
| 558 | P198 with short CD8 hinge | 198-22-8-26-28-3z |
| 559 | P198 with intermediate CD8 hinge | 198-22-8-46-28-3z |
| 560 | P198 with long CD8 hinge | 198-22-8-66-28-3z |
| 561 | m971 with CD28 hinge | m971-22-28-40-28-3z |
| 562 | m971 with short CD8 hinge | m971-8-26-28-3z |
| 563 | m971 with intermediate CD8 hinge | m971-8-46-28-3z |
| 564 | m971 with long CD8 hinge | m971-8-66-28-3z |
| 565 | Human CD8 alpha chain isoform 1 | human |
| | precursor | |
| 566 | Albumin VHH CD1 | synthetic construct |
| 567 | Albumin VHH CD2 | synthetic construct |
| 568 | Albumin VHH CD3 | synthetic construct |
| 569 | frame sequence of VHH1 type ISVD | |
| 570 | Human CD8a, full sequence | human |
| 571 | Anti-CD8a CDR1 Consensus | Synthetic construct |
| | sequence 1 | |
| 572 | Anti-CD8a CDR2 Consensus | Synthetic construct |
| | sequence 1 | |
| 573 | Anti-CD8a CDR3 Consensus | Synthetic construct |
| | sequence 1 | |
| 574 | Anti-CD8a CDR2 Consensus | Synthetic construct |
| | sequence 1 | |

Sequences

| SEQ | |
|---|---|
| ID | |
| NO | Sequence |
| 1 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAK |
| | YSRPDPSEGHVDLDYWGQGTLVTVSS |
| 2 | EVQLVESGGGVVQPGGSIRLSCAASGFTFEDYAIGWFRQAPGKEREEVSCIRTYDEQTYYADSVKGRFTISRDNAKNTVYLQMNSLRPEDTALYYCAAGSYYACAE |
| | YSRPDPSEGHVDLDYWGQGTLVTVSS |
| 3 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAY |
| | YSRPDPSEGHVDLDYWGQGTLVTVSS |
| 4 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | KYSRPDPSEGHVDLDYWGQGTLVTVSS |
| 5 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | EYSRPDPSEGHVDLDYWGQGTLVTVSS |
| 6 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | YYSRPDPSEGHVDLDYWGQGTLVTVSS |
| 7 | EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAK |
| | YSRPDPSEGHVDLDYWGQGTLVTVSS |
| 8 | EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAE |
| | YSRPDPSEGHVDLDYWGQGTLVTVSS |
| 9 | EVQLVESGGGVVQPGGSLRLSCAASGFTFEDYAIGWFRQAPGKEREEVSCIRTYDEQTYYADSVKGRFTISRDNAKNTVSLQMNSIRPEDTALYYCAAGSYYACAY |
| | YSRPDPSEGHVDLDYWGQGTLVTVSS |
| 10 | EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYT |
| | VRKYRYWGQGTLVTVSS |
| 11 | EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYT |
| | VRKYRYWGQGTLVTVSS |
| 12 | EVQLVESGGGVVQPGGSLRLSCAASGFTFSDYGVGWFRQAPGKGREFVADIDWEGEHTSYADSVKGRFTISRDNAKNTAYLQMNSLRPEDTALYYCAYDALPYT |
| | VRKYRYWGQGTLVTVSS |
| 13 | EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAADALPYT |
| | LRKYRYWGQGTLVTVSS |
| 14 | EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAEDALPYT |
| | LRKYRYWGQGTLVTVSS |
| 15 | EVQLVESGGGVVQPGGSLRLSCAASGSTFSDYGVGWFRQAPGKGREFVADIDWEGEHTSYADSVKGRFTISRDNAKNTAYLQMNSLRPEDTALYYCAYDALPYT |
| | LRKYRYWGQGTLVTVSS |
| 16 | EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYT |
| | VRKYRYWGQGTLVTVSS |
| 17 | EVQLVESGGGVVQPGGSLRLSCAASGSTFSDYGVGWFRQAPGKGREFVADIDWEGEHTSYADSVKGRFTISRDNAKNTADLQMNSLRPEDTALYYCAADALPYT |
| | LRKYRYWGQGTLVTVSS |
| 18 | EVQLVESGGGVVQPGGSLRLSCAASGSTFSDYGVGWFRQAPGKGREFVADIDWEGEHTSYADSVKGRFTISRDNAKNTADLQMNSLRPEDTALYYCAEDALPYT |
| | LRKYRYWGQGTLVTVSS |
| 19 | EVQLVESGGGVVQPGGSLRLSCAASGSTFSDYGVGWFRQAPGKGREFVADIDWEGEHTSYADSVKGRFTISRQNAKNTADLINSURPEDTALYYCAYDALPYT |
| | LRKYRYWGQGTLVTVSS |
| 20 | EVQLVESGGGVVQPGGSLRLSCAASGSTFSDYGVGWFRQAPGKGREFVADIDWEGEHTSVADSVKGRFTISRDNAKNTADLQMNSLRPEDTALYYCAEDALPYT |
| | VRKYRYWGQGTLVTVSS |
| 21 | EVQLVESGGGVVQPGGSLRLSCAASGSTFSDYGVGWFRQAPGKGREFVADIDWEGEHTSYADSVKGRFTISRDNAKNTADLQMNSLRPEDTALYYCAYDALPYT |
| | VRKYRYWGQGTLVTVSS |
| 22 | EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYT |
| | LRKYRYWGQGTLVTVSS |
| 23 | EVQLVESGGGVVQPGGSURLSCAASGSTFSDYGVGWFRQAPGKGREFVADIDWEGEHTSYADSVKGRFTISRDNAKNTASLQMNSLRPEDTALYYCAEDALPYT |
| | LRKYRYWGQGTLVTVSS |
| 24 | EVQLVESGGGVVQPGGSURLSCAASGSTFSDYGVGWFRQAPGKGREFVADIDWEGEHTSYADSVKGRFTISRDNAKNTASLQMNSLRPEDTALYYCAYDALPYT |
| | LRKYRYWGQGTLVTVSS |
| 25 | EVQLVESGGGVVQPGGSLRLSCAASGSTFSDYGVGWFRQAPGKGREFVADIDWEGEHTSYADSVKGRFTISRDNAKNTASLQMNSLRPEDTALYYCAADALPYT |
| | VRKYRYWGQGTLVTVSS |
| 26 | EVQLVESGGGVVQPGGSLRLSCAASGSTFSDYGVGWFRQAPGKGREFVADIDWEGEHTSYADSVKGRFTISRDNAKNTASLQVINSLRPEDTALYYCAEDALPYT |
| | VRKYRYWGQGTLVTVSS |
| 27 | EVQLVESGGGVVQPGGSLRLSCAASGSTFSDYGVGWFRQAPGKGREFVADIDWEGEHTSYADSVKGRFTISRDNAKNTASLQMNSLRPEDTALYYCAYDALPYT |
| | VRKYRYWGQGTLVTVSS |
| 28 | EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAK |
| | YSRPDPSENHVDMDYWGKGTLVTVSSGCC |
| 29 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAK |
| | YSRPDPSEGHVDLDYWGQGTLVTVSSGGC |
| 30 | EVQLVESGGGVVQPGGSIRISCAASGFTFEDYAIGWFRQAPGKEREEVSCIRTYDEQTYYADSVKGRFTISRDNAKNTVYLQMNSLRPEDTALYYCAAGSYYACAE |
| | YSRPDPSEGHVDLDYWGQGTLVTVSSGGC |
| 31 | EVQLVESGGGVVQPGGSIRLSCAASGFTFEDYAIGWFRQAPGKEREEVSCIRTYDEQTYYADSVKGRFTISRDNAKNTVYLQMNSLRPEDTALYYCAAGSYYACAY |
| | YSRPDPSEGHVDLDYWGQGTLVTVSSGGC |
| 32 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | KYSRPDPSEGHVDLDYWGQGTLVTVSSGGC |
| 33 | EVQLVESGGGVVQPGGSLRLSCAASGFTFEDYAIGWFRQAPGKEREEVCIRTYDEQTYYADSVKGRITISRDNAKNTVDLQMNSLRPEDTALYYCAAGSYYACA |
| | EYSRPDPSEGHVDLDYWGQGTLVTVSSGGC |
| 34 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | YYSRPDPSEGHVDLDYWGQGTLVTVSSGGC |
| 35 | EVQLVESGGGVVQPGGSLRLSCAASGFTFEDYAIGWFRQAPGKEREEVSCIRTYDEQTYYADSVKQFTISRDNAKNTVSLQMNSLRPEDTALYYCAAGSYYACAK |
| | YSRPDPSEGHVDLDYWGQGTLVTVSSGGC |
| 36 | EVQLVESGGGVVQPGGSLRLSCAASGFTFEDYAIGWFRQAPGKEREEVSCIRTYDEQTYYADSVKGRFTISRDNAKNTVSLQMNSIRPEDTALYYCAAGSYYACAE |
| | YSRPDPSEGHVDLDYWGQGTLVTVSSGGC |
| 37 | DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGP |
| | MGCVDLSTLSFGHWGQGTQVTVSITTSFVPVFLPAKPTTTPAPRPPTPAPTIASQPLSLRPEASRPAAGGAVHTRGLDFAGGCGGHHHHHH |
| 38 | QVQLVESGGGVVQPGRSLRISCAASGFTFSDFGMINWVRQAPGKGLEWVALIYYDGSNKFYADSVKGRFTISRDNSKNTLYLQMNSLRAEDTAVYYCAKPHYDG |
| | YYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKP |
| | SNTKVDKKVEPKSSDKTHTC |
| 39 | QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGT |
| | KLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVA |
| | PAESS |
| 40 | EVQLVESGGGLVQPGGSLKLSCAASGFTENKYAMNWVRQAPGKGLEWVARIRSKYNNYATYYADSVKDRFTISRDDSKNTAYLQMNNLKTEDTAVYYCVRHGN |
| | FGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNV |
| | NHKPSNTKVDKKVEPKSSDKTHTC |
| 41 | QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGT |
| | KLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVA |
| | PAESS |
| 42 | QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGYY |
| | VFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNT |
| | KVDKKVEPKSSDKTHTC |
| 43 | DIVMTQSPSSLSASVGDRVTITCRTSRSISQALAWYQEKPGKAPKLLIYSGSTLQSGVPSRFSGSGSGTDFTLTISSLQPEDFATYYCQQHNENPLTFGQGTKVEIKRT |
| | VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES |
| 44 | EVQLVESGGGVVQPGGSLRLSCAASGFTFEDYAIGWFRQAPGKEREEVSCIRTYDEQTYVADSVKGRFTISRDNAKNTVSLQMNSLRPEDTALYYCAAGSYYACAY |
| | YSRPDPSEGHVDLDYWGQGTLVTVSSGGC |
| 45 | AAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAAA |
| | AAAAAAAAAAAAAA |
| 46 | ATGGACATGAGGGTGCCCGCCCAGCTCCTGGGCCTGCTGTTACTGTGGCTGCCCGGCGCTAAGTGCCAGGTACAGCTCGTGGAGAGCGGCGGAGGCGTG |
| | GTGCAGCCCGGCAGGAGCCTGAGGTTAAGCTGCGCCGCCAGCGGCTTCACCTTCAGCAGCTACGGCATGCACTGGGTGAGACAGGCCCCCGGCAAGGGC |
| | TTGGAGTGGGTGGCCATCATCTACTACGACGGCAGCAAGAAGTACTACGCCGACAGCGTGAAGGGAAGGTTCACCATCAGCAGGGACAACAGCAAGAAC |
| | ACCCTGTACCTGCAGATGAACAGCCTGAGGGCCGAGGACACCGCCGTGTACTACTGCGCCAGGGAGCTGACCGGCGACGCCTTCGACATCTGGGGCCAGG |
| | GAACCAAGGTGACCGTCAGCAGCGGGGGGGGAGGATCTGGCGGAGGAGGATCAGGAGGCGGCGGATCTGGGGGGGGCGGAAGCGAGATCGTGCTGAC |
| | CCAGACCCCCAGCAGCCTGAGCGCCAGCCTGGGCGACAGGGTGACCATCAGCTGCAGGGCCTCCCAGAGCATCGGCTCCAGCCTGCACTGGTACCAGCAG |
| | AAGCCCGACCAGAGCCCCAAGCTGCTGATCAAGTACGCCAGCCAGAGCTTCAGCGGCGTGCCCAGCAAGTTCATCGGCAGCGGAAGCGGCACCGACTTCA |
| | CCTTGACCATCAACAGCCTGGAGGCCGAAGACGCCGCCACCTTTTATTGCCACCAGAGCAGCACCCTGCCCTACACCTTCGGCCAGGGCACCAAGCTGGAG |
| | ATCAAGGCCGCCGCCATCGAGGTGATGTACCCTCCCCCTTACCTTGACAACGAGAAGAGCAACGGCACCATCATCCACGTGAAGGGCAAGCACCTGTGCCC |
| | CAGCCCCCTGTTCCCCGGCCCCTCTAAGCCCTTCTGGGTCCTGGTGGTGGTTGGCGGCGTGCTGGCCTGCTACAGCCTGCTGGTGACCGTGGCCTTCATCAT |
| | CTTCTGGGTGAGGAGCAAAAGGAGCAGGCTGCTTCACAGCGACTACATGAACATGACCCCCAGGCGACCCGGCCCCACCAGGAAGCACTACCAGCCCTAT |
| | GCCCCACCCAGGGACTTCGCCGCCTACAGGTCTAGGGTGAAGTTCAGCAGGAGCGCCGACGCCCCCGCCTACCAGCAGGGCCAGAACCAGCTGTACAATG |
| | AACTGAACCTGGGCAGGAGGGAGGAGTACGACGTGCTGGACAAGAGGAGGGGAAGGGACCCCGAGATGGGGGGCAAGCCCAGGAGGAAGAACCCCCA |
| | GGAAGGCCTGTACAACGAGCTCCAGAAGGATAAAATGGCCGAGGCCTACAGCGAAATCGGCATGAAGGGCGAGAGGAGGAGGGGCAAGGGCCACGAC |
| | GGCCTTTACCAGGGCCTGAGCACCGCCACCAAGGACACCTACGACGCCCTGCACATGCAGGCCCTGCCCCCTAGGTAATGATGA |
| 47 | ATGGACATGAGGGTGCCCGCCCAGCTGCTGGGCCTGTTACTCCTTTGGCTGCCCGGCGCCAAGTGCCAGGTGCAGCTGGTTGÅGAGCGGAGGCGGCGTG |
| | GTGCAGCCCGGCAGGAGCCTGAGGTTAAGCTGCGCCGCCAGCGGCTTCACCTTCAGCAGCTACGGCATGCACTGGGTGAGACAGGCCCCCGGCAAGGGC |
| | TTGGAGTGGGTGGCCATCATCTACTACGACGGCAGCAAGAAGTACTACGCCGACAGCGTGAAGGGCAGGTTCACCATCAGCAGGGACAACAGCAAGAAC |
| | ACCCTGTACCTGCAGATGAACAGCCTGAGAGCCGAGGACACCGCCGTGTACTACTGCGCCAGGGAGCTGACCGGCGACGCCTTCGACATCTGGGGCCAGG |
| | GAACCAAGGTGACCGTGAGCAGCGGCGGCGGAGGATCTGGCGGAGGAGGCTCAGGAGGAGGTGGAAGCGGGGGCGGCGGCTCTGAGATCGTGCTGAC |
| | CCAGACCCCCAGCTCTCTGAGCGCCAGCCTGGGCGACAGGGTGACCATCAGCTGCAGGGCCTCCCAGAGCATCGGCTCTAGCCTGCACTGGTACCAGCAG |
| | AAGCCCGACCAGAGCCCCAAGCTGCTGATCAAGTACGCCAGCCAGAGCTTCAGCGGCGTGCCCAGCAAGTTCATCGGCAGCGGGAGCGGCACCGACTTCA |
| | CCTTGACCATCAACAGCCTGGAGGCCGAGGACGCCGCCACCTTCTACTGCCACCAGAGCAGCACCCTGCCCTACACCTTCGGCCAGGGCACCAAGCTGGAG |
| | ATCAAGGCCAGCAGCTTAAGACCCGAAGCCTGCAGGCCCGCCGCCGGAGGAGCTGTTCACACCAGGGGCTTGGATTTTGCTTGCGATATCTACATCTGGGC |
| | CCCCTTAGCCGGCACCTGCGGCGTGCTGCTGCTGTCTCTGGTGATCACCTTATACTGTAACCACAGGAACGTGAGGAGCAAGAGAAGCAGGCTGCTGCACA |
| | GCGACTACATGAACATGACCCCTAGGAGGCCCGGCCCCACCAGGAAGCACTACCAGCCCTACGCCCCACCCAGGGACTTCGCCGCCTACAGGAGCAGGGT |
| | GAAGTTCAGCAGGAGCGCCGACGCCCCCGCCTACCAGCAGGGCCAGAACCAGCTGTACAATGAGCTGAACCTGGGCAGGAGGGAGGAGTACGACGTGCT |
| | TGACAAGAGGAGGGGAAGGGACCCCGAAATGGGGGGCAAGCCCAGGAGGAAAAACCCCCAAGAGGGCCTGTACAACGAGCTCCAGAAGGACAAGATG |
| | GCCGAGGCCTACAGCGAGATCGGCATGAAGGGCGAGAGGAGGAGGGGCAAGGGCCACGACGGCCTGTACCAGGGCCTTAGCACCGCCACCAAGGACAC |
| | CTACGACGCCCTGCACATGCAGGCCCTGCCCCCTAGGTAATGATGA |
| 48 | ATGGACATGAGGGTGCCCGCCCAGCTGCTGGGACTGCTGCTCCTTTGGCTGCCTGGCGCCAAGTGCCAGGTGCAGCTGGTGGAGAGCGGCGGAGGAGTG |
| | GTGCAGCCCGGCAGGAGCCTGAGACTGAGCTGCGCCGCCAGCGGCTTCACCTTCAGCAGCTACGGCATGCACTGGGTGAGGCAGGCCCCCGGCAAGGGC |
| | TTGGAGTGGGTGGCCATCATCTACTACGACGGCAGCAAGAAGTACTACGCCGACAGCGTGAAGGGCAGGTTCACCATCAGCAGGGACAACAGCAAGAAC |
| | ACCCTGTATCTGCAGATGAACAGCCTGAGGGCCGAGGATACCGCCGTGTACTACTGCGCCAGGGAGCTGACCGGCGACGCCTTCGACATCTGGGGCCAGG |
| | GAACCAAGGTGACCGTGAGCAGCGGAGGGGGAGGATCTGGAGGTGGCGGTAGCGGAGGAGGCGGAAGCGGCGGTGGCGGCAGCGAAATTGTGCTGAC |
| | CCAGACCCCCAGCAGCCTCAGCGCCAGCCTGGGCGACAGGGTGACCATCAGCTGCAGGGCCTCCCAGAGCATCGGCTCCAGCCTGCACTGGTACCAGCAG |
| | AAGCCCGACCAGAGCCCCAAGCTGCTGATCAAGTACGCCAGCCAAAGCTTCAGCGGCGTGCCCAGCAAGTTCATCGGAAGCGGATCCGGAACCGACTTCA |
| | CCTTAACCATCAACAGCCTGGAGGCCGAGGACGCCGCCACCTTCTACTGTCACCAGAGCAGCACCCTGCCCTACACCTTTGGCCAGGGCACCAAGCTGGAA |
| | ATCAAAGCCAGCACCACCACCCCCGCTCCTAGGCCTCCCACACCCGCCCCCACAATTGCTAGCCAGCCTTTGAGCCTGAGGCCTGAAGCTTGTAGACCCGCT |
| | GCCGGAGGCGCCGTGCACACCAGGGGCCTGGACTTCGCTTGCGACATCTACATCTGGGCCCCCTTGGCCGGCACCTGCGGCGTGCTGCTGCTGAGCCTGG |
| | TGATCACCCTGTACTGCAACCACAGGAACGTGAGGAGCAAGAGGAGCAGGCTGCTGCACAGCGACTACATGAACATGACCCCCAGGCGACCCGGCCCCAC |
| | CAGGAAGCACTACCAGCCCTACGCCCCACCCAGGGACTTCGCCGCCTATAGGAGCAGGGTGAAGTTCAGCAGGAGCGCCGACGCCCCCGCCTACCAGCAG |
| | GGCCAGAACCAGCTGTACAATGAGCTGAACCTGGGCAGGAGGGAGGAGTACGACGTGTTAGACAAGAGGAGGGGAAGGGATCCCGAGATGGGGGGCA |
| | AGCCCAGGAGGAAGAACCCCCAGGAAGGCCTGTACAACGAGCTCCAGAAGGACAAGATGGCTGAGGCCTACAGCGAAATCGGAATGAAGGGCGAGAGG |
| | AGGAGGGGCAAGGGCCACGACGGCCTGTATCAGGGCCTGAGCACCGCCACCAAGGACACCTACGACGCCCTGCACATGCAGGCCCTGCCCCCTAGGTAAT |
| | GATGA |
| 49 | ATGGACATGAGGGTGCCCGCCCAACTGCTTGGCCTGCTGCTCCTTTGGCTGCCCGGCGCCAAGTGCCAGGTGCAGCTGGTGGAGTCTGGCGGAGGCGTGG |
| | TGCAGCCCGGAAGGAGCCTGAGACTGAGCTGCGCCGCCAGCGGCTTCACCTTCAGCAGCTACGGCATGCACTGGGTGAGGCAGGCCCCCGGCAAGGGCT |
| | TGGAGTGGGTGGCCATCATCTACTACGACGGCAGCAAGAAGTACTACGCCGACAGCGTGAAGGGCAGGTTCACCATCAGCAGGGACAACAGCAAGAACA |
| | CCCTGTATCTGCAGATGAACAGCCTGAGGGCCGAGGATACCGCCGTGTACTACTGCGCCAGGGAGCTGACCGGCGACGCCTTCGACATCTGGGGCCAGGG |
| | AACCAAGGTGACCGTGAGCAGCGGCGGAGGAGGATCTGGAGGCGGCGGATCAGGAGGTGGGGGAAGCGGGGGAGGCGGCAGCGAAATCGTGCTGAC |
| | CCAGACCCCCAGCAGCCTCAGCGCCAGCCTGGGCGACAGGGTGACCATCAGCTGCAGGGCCTCCCAGAGCATCGGCTCCAGCCTGCACTGGTACCAGCAG |
| | AAGCCCGACCAGAGCCCCAAGCTGCTGATCAAGTACGCCAGCCAAAGCTTCAGCGGCGTGCCCAGCAAGTTCATCGGCAGCGGAAGCGGCACCGACTTCA |
| | CCTTGACCATCAACAGCCTGGAGGCCGAGGACGCCGCCACCTTCTACTGCCACCAGAGCAGCACCCTGCCCTACACCTTCGGCCAGGGCACCAAGCTGGAG |
| | ATCAAGGCCAGCCTGAGCAACAGCATCATGTACTTCAGCCACTTCGTGCCCGTGTTCCTGCCCGCCAAGCCTACCACAACACCCGCCCCCAGGCCCCCAACA |
| | CCTGCTCCCACAATCGCCAGCCAGCCCTTATCCTTAAGGCCCGAGGCCTGCAGACCCGCTGCCGGAGGCGCTGTGCATACCAGGGGACTGGACTTCGCTTG |
| | CGACATCTACATCTGGGCCCCCTTAGCCGGAACATGCGGCGTGCTGCTGCTGAGCCTGGTGATCACCCTGTACTGCAACCACAGGAACGTGAGGAGCAAG |
| | AGGAGCAGGCTGCTGCACAGCGACTACATGAACATGACCCCCAGGCGACCCGGCCCTACCAGGAAACATTATCAGCCCTACGCCCCACCCAGGGACTTCGC |
| | CGCCTACAGGAGCAGGGTGAAGTTCAGCAGGAGCGCCGACGCCCCCGCCTACCAGCAGGGCCAGAACCAGCTGTACAATGAGCTGAACCTGGGCAGGAG |
| | GGAGGAGTACGACGTGCTGGACAAGAGGAGGGGAAGGGACCCCGAGATGGGCGGAAAGCCCAGGAGGAAGAACCCCCAGGAGGGCCTGTACAACGAG |
| | CTCCAGAAGGACAAGATGGCCGAGGCCTATAGCGAGATCGGCATGAAGGGCGAGAGGAGGAGGGGCAAGGGCCACGACGGCCTGTACCAAGGCCTGA |
| | GCACCGCCACCAAGGACACCTACGACGCTCTTCACATGCAGGCCCTGCCCCCTAGGTAATGATGA |
| 50 | ATGGACATGAGGGTGCCTGCCCAGCTGCTGGGCCTGCTGCTGCTGTGGCTTCCTGGCGCCAAGTGCCAGGTGCAGCTGGTGCAGAGGCGCCGAGGTG |
| | AAGAAGCCTGGCGCCTCCGTGAAGGTGAGCTGCAAGGCCAGCGGCTACACCTTCACCGGCTACTACATCCACTGGGTGAGACAGGCCCCCGGCCAGGGAC |
| | TGGAGTGGATGGGCTGGATCAACCCCAACAGCGGCGGCACCAACTACGCCCAGAAGTTCCAGGGCAGGGTGACAATGACCAGGGACACCAGCATCGGCA |
| | CCGCCTACATGGAGCTGAGCAGGCTCAGGAGCGACGACACCGCCATCTACTACTGCACCAGGGAGGACACCTGGAACTACATCGACTACTGGGGCCAGGG |
| | AACCCTGGTGACAGTGAGCAGCGGAGGTGGCGGAAGCGGCGGCGGAGGATCAGGGGGAGGTGGAAGTGGCGGAGGAGGCAGCGACATCCAGATGAC |
| | CCAGAGCCCCAGCTCCCTGAGCGCCAGCGTGGGCGACAGGGTGACCATCACCTGCAGGGCCTCCCAGAGCATCAGCAGCCACCTGAACTGGTACCAGCAG |
| | AAGCCCGGCAAGGCCCCCAAGCTGCTGATCTACGCCGCCAGCAGCCTCCACAGCGGCGTGCCCAGCAGGTTCTCTGGCTCTGGCAGCGGCACCGACTTCAC |
| | CTTAACAATTTCTAGCCTGCAACCCGAGGACTTCGCTACCTACTGCTGCCAGCAGAGCTACAGCACCCCCAGAACCTTTGGCCAGGGCACCAAGTGGAGA |
| | TCAAGGCCGCCGCCATCGAGGTGATGTACCCTCCCCCTTACCTGGACAACGAGAAGAGCAACGGCACCATCATCCACGTGAAGGGCAAGGACCTGTGCCCC |
| | AGCCCCCTGTTCCCCGGCCCCTCTAAGCCCTTCTGGGTCCTGGTGGTGGTGGGCGGCGTGCTGGCCTGCTATAGCCTGCTGGTGACCGTGGCCTTCATCATC |
| | TTCTGGGTGAGGAGCAAGAGGAGCAGGCTGCTGCACAGCGATTACATGAACATGACCCCCAGGCGACCCGGCCCTACCAGGAAGCACTACCAGCCCTACG |
| | CCCCACCCAGGGACTTCGCCGCCTACAGGAGCAGGGTGAAGTTCAGCAGGAGCGCCGACGCCCCCGCTTACCAGCAGGGCCAGAACCAGCTGTACAATGA |
| | GCTGAACCTGGGCAGGAGGGAGGAGTACGACGTGCTGGACAAGAGGAGGGGAAGGGACCCCGAGATGGGCGGCAAGCCCAGGAGGAAGAACCCCCAG |
| | GAGGGCCTGTACAACGAGCTCCAGAAAGACAAGATGGCCGAGGCCTACAGCGAGATCGGCATGAAGGGCGAGAGGAGGAGGGGCAAGGGCCACGACG |
| | GCCTGTACCAGGGCCTGAGCACCGCCACCAAGGATACCTACGATGCCCTGCACATGCAGGCCCTGCCTCCTAGGTAATGATGA |
| 51 | ATGGACATGAGGGTGCCCGCCCAGCTGCTGGGCCTGCTGTTGCTTTGGCTGCCCGGCGCTAAGTGCCAGGTGCAGCTGGTGCAGAGCGGCGCCGAGGTG |
| | AAGAAGCCTGGCGCCTCCGTGAAGGTGAGCTGCAAGGCCAGCGGCTACACCTTCACCGGCTACTACATCCACTGGGTGAGGCAGGCCCCCGGCCAGGGAC |
| | TGGAGTGGATGGGCTGGATCAACCCCAACAGCGGCGGCACCAACTACGCCCAGAAGTTCCAGGGCAGGGTGACAATGACCAGGGACACCAGCATCGGCA |
| | CCGCCTACATGGAGCTGAGCAGGCTCAGGAGCGACGACACCGCCATCTACTACTGCACCAGGGAGGACACCTGGAACTACATCGACTACTGGGGCCAGGG |
| | AACCCTGGTGACCGTGAGCAGCGGAGGGGGAGGAAGCGGCGGAGGAGGCAGTGGAGGAGGAGGATCCGGAGGCGGCGGAAGCGACATCCAGATGAC |
| | CCAGAGCCCCAGCTCCCTGAGCGCCAGCGTGGGCGACAGGGTGACCATCACCTGCAGGGCCTCTCAGAGCATCAGCTCTCACCTGAATTGGTACCAGCAG |
| | AAGCCCGGCAAGGCCCCCAAGCTGCTGATCTACGCCGCCAGCAGCTTACACAGCGGCGTGCCCAGCAGGTTTTCTGGCAGCGGAAGCGGCACCGACTTCA |
| | CCTTAACAATCTCCAGCCTGCAGCCCGAGGACTTTGCTACCTACTGCTGCCAGCAGAGTCTACAGCACCCCCAGAACCTTCGGCCAGGGCACCAAGGTGGAA |
| | ATCAAGGCCTCTTCTCTGAGACCCGAGGCTTGCAGACCCGCCGCTGGAGGCGCCGTTCATACCAGGGGCCTGGACTTCGCTTGCGACATCTACATCTGGGC |
| | CCCCTTGGCCGGCACCTGCGGCGTGCTGCTGCTGAGCCTGGTGATCACCCTGTACTGCAACCACAGGAACGTGAGGAGCAAGAGGAGCAGGCTGCTGCAT |
| | AGCGACTACATGAACATGACCCCCAGGCGACCCGGCCCCACCAGGAAGCACTACCAGCCCTACGCCCCACCCAGGGACTTCGCCGCCTACAGGAGCAGGG |
| | TGAAGTTCAGCAGGAGCGCTGATGCCCCCGCCTACCAGCAGGGCCAGAACCAGCTGTACAATGAGCTGAATCTGGGCAGGAGGGAGGAGATACGACGTGC |
| | TGGACAAGAGGAGGGGAAGGGACCCCGAGATGGGCGGCAAGCCCAGGAGGAAGAACCCCCAGGAGGGCCTGTACAACGAGCTCCAGAAGGACAAGAT |
| | GGCCGAGGCCTACAGCGAGATCGGCATGAAGGGCGAGAGGAGGAGAGGCAAGGGCCACGACGGCCTGTACCAGGGCCTGAGCACCGCCACCAAAGACA |
| | CCTACGACGCCCTGCACATGCAGGCCCTGCCCCCTAGGTAATGATGA |
| 52 | ATGGACATGAGGGTGCCCGCCCAGCTGCTGGGACTGCTGCTCCTTTGGCTGCCCGGCGCTAAGTGCCAGGTGCAGCTGGTGCAGAGCGGCGCCGAGGTG |
| | AAGAAGCCTGGCGCCTCCGTGAAGGTGAGCTGCAAGGCCAGCGGCTACACCTTCACCGGCTACTACATCCACTGGGTGAGGCAGGCCCCCGGCCAGGGAC |
| | TGGAGTGGATGGGCTGGATCAACCCCAACAGCGGCGGCACCAACTACGCCCAGAAGTTCCAGGGCAGAGTGACCATGACCAGGGACACCAGCATCGGCA |
| | CCGCCTACATGGAGCTGAGCAGGCTCAGGAGCGACGACACCGCCATCTACTACTGCACCAGGGAGGACACCTGGAACTACATCGACTACTGGGGCCAGGG |
| | CACCCTGGTGACCGTGAGCAGCGGAGGAGGCGGATCTGGCGGCGGAGGATCAGGAGGGGGAGGAAGCGGAGGCGGAGGCAGCGACATCCAGATGACC |
| | CAGAGCCCCAGCTCCCTGAGCGCCAGCGTGGGCGACAGGGTTACCATTACCTGCAGGGCCTCTCAGTCTATCAGCTCTCATCTGAACTGGTACCAGCAGAA |
| | GCCCGGCAAGGCCCCTAAGCTGCTGATCTATGCCGCCAGCAGCCTCCACAGCGGCGTGCCTAGCAGGTTCTCTGGCAGCGGAAGTGGCACCGACTTCACCT |
| | TAACCATCAGCTCACTGCAGCCCGAGGACTTCGCTACCTACTGCTGCCAGCAGAGCTACAGCACCCCCAGAACCTTCGGACAGGGCACCAAGGTGGAGATC |
| | AAGGCCTCCACAACAACACCCGCTCCCAGGCCCCCTACCCCCGCCCCCACAATTGCCAGCCAACCCCTCAGCCTGAGACCCGAAGCCTGTAGACCTGCCGCA |
| | GGGGGAGCCGTGCACACCAGGGGCCTGGACTTCGCATGCGACATCTACATCTGGGCCCCCTTAGCCGGCACCTGCGGCGTGCTGCTGCTGAGCCTTGTGA |
| | TCACCCTGTACTGCAACCACAGGAACGTGAGGAGCAAGAGGAGCAGGCTGCTGCACAGCGACTACATGAACATGACCCCCAGGCGACCCGGCCCCACCAG |
| | GAAGCATTACCAGCCCTACGCCCCACCCAGGGACTTCGCCGCCTACAGGAGCAGGGTGAAGTTCAGCAGGAGCGCCGACGCCCCTGCCTACCAGCAGGGC |
| | CAGAACCAGCTGTACAATAGCTGAACCTGGGCAGGAGGGAGGAGTATGACGTGCTGGACAAGAGGAGGGGAAGGGACCCCGAGATGGGCGGCAAGC |
| | CCAGGAGGAAGAACCCCCAGGAGGGCCTGTATAACGAGCTCCAGAAGGACAAGATGGCCGAAGCCTACAGCGAGATCGGCATGAAGGGCGAGAGGAGG |
| | AGGGGCAAGGGCCATGACGGCCTGTACCAGGGCCTGAGCACCGCCACCAAGGACACCTACGACGCCCTGCACATGCAGGCCCTGCCCCCTAGGTAATGATGA |
| 53 | ATGGACATGAGGGTGCCCGCCCAGCTGCTGGGACTGCTGCTCCTTTGGCTGCCCGGCGCTAAGTGCCAGGTGCAGCTGGTGCAGAGCGGCGCCGAGGTG |
| | AAGAAGCCTGGCGCCTCCGTGAAGGTGAGCTGCAAGGCCAGCGGCTACACCTTCACCGGCTACTACATCCACTGGGTGAGGCAGGCCCCCGGCCAGGGAC |
| | TGGAGTGGATGGGCTGGATCAACCCCAACAGCGGCGGCACCAACTACGCCCAGAAGTTCCAGGGCAGAGTGACCATGACCAGGGACACCAGCATCGGCA |
| | CCGCCTACATGGAGCTGAGCAGGCTCAGGAGCGACGACACCGCCATCTACTACTGCACCAGGGAGGACACCTGGAACTACATCGACTACTGGGGCCAGGG |
| | CACCCTGGTGACCGTGAGCAGCGGAGGAGGCGGATCTGGCGGCGGAGGATCAGGAGGGGGAGGAAGCGGAGGCGGAGGCAGCGACATCCAGATGACC |
| | CAGAGCCCCAGCTCCCTGAGCGCCAGCGTGGGCGACAGGGTTACCATTACCTGCAGGGCCTCTCAGTCTATCAGCTCTCATCTGAACTGGTACCAGCAGAA |
| | GCCCGGCAAGGCCCCTAAGCTGCTGATCTATGCCGCCAGCAGCCTCCACAGCGGCGTGCCTAGCAGGTTCTCTGGCAGCGGAAGTGGCACCGACTTCACCT |
| | TAACCATCAGCTCACTGCAGCCCGAGGACTTCGCTACCTACTGCTGCCAGCAGAGCTACAGCACCCCCAGAACCTTCGGACAGGGCACCAAGGTGGAGATC |
| | AAGGCCTCCACAACAACTAGCATCATGTACTTCAGCCATTTCGTGCCCGTGTTTCTTCCCGCTAAACCCACCACTACACCCGCTCCCAGGCCCCCTACCCCCG |
| | CCCCCACAATTGCCAGCCAACCCCTCAGCCTGAGACCCGAAGCCTGTAGACCTGCCGCAGGGGGAGCCGTGCACACCAGGGGCCTGGACTTCGCATGCGA |
| | CATCTACATCTGGGCCCCCTTAGCCGGCACCTGCGGCGTGCTGCTGCTGAGCCTTGTGATCACCCTGTACTGCAACCACAGGAACGTGAGGAGCAAGAGGA |
| | GCAGGCTGCTGCACAGCGACTACATGAACATGACCCCCAGGCGACCCGGCCCCACCAGGAAGCATTACCAGCCCTACGCCCCACCCAGGGACTTCGCCGCC |
| | TACAGGAGCAGGGTGAAGTTCAGCAGGAGCGCCGACGCCCCTGCCTACCAGCAGGGCCAGAACCAGCTGTACAATGAGCTGAACCTGGGCAGGAGGGA |
| | GGAGTATGACGTGCTGGACAAGAGGAGGGGAAGGGACCCCGAGATGGGGGCAAGCCCAGGAGGAAGAACCCCCAGGAGGGCCTGTATAACGAGCTC |
| | CAGAAGGACAAGATGGCCGAAGCCTACAGCGAGATCGGCATGAAGGGCGAGAGGAGGAGGGGCAAGGGCCATGACGGCCTGTACCAGGGCCTGAGCA |
| | CCGCCACCAAGGACACCTACGACGCCCTGCACATGCAGGCCCTGCCCCCTAGGTAATGATGA |
| 54 | ATGGACATGAGGGTGCCTGCCCAGTTGTTGGGCCTGCTGCTGCTGTGGCTGCCCGGCGCCAAGTGCCAGGTTACCCTGAGGGAGTCTGGCCCCGCCCTG |
| | TGAAGCCCACCCAGACCTTAACCCTGACCTGCACCTTCAGCGGCATCAGCCTGACCACCAGCGGCATGTGCGTGAGCTGGATCAGGCAGCCCCCAGGCAAG |
| | GCCCTGGAGTGGCTGGCCCTGATCGACTGGGACGGCGACAACTACTACAGCACCAGCCTGAAGACCAGGCTGACCATCTCCAAGGACACCAGCAAGAACC |
| | AGGTGGTGCTGACCATGGCCAGCATGGACCCCGTGGACACCGCCACATACTACTGCGCCAGGATGGGCTACACCTACGGCTACGACGCTTTCGACATCTGG |
| | GGCCAGGGCACCATGGTGACAGTGAGCAGCGGCGGTGGAGGATCTGGCGGAGGCGGAAGCGGAGGAGGCGGATCTGGGGGCGGAGGAAGCGAGATT |
| | GTGCTGACCCAGACCCCCAGCAGCCTCAGCGCCAGCCTGGGCGACAGGGTGACCATCAGCTGCAGGGCCTCTCAGAGCATCGGCAGCCTGTTACACTGGT |
| | ACCAGCAGAAGCCCGACCAGAGCCCCAAGCTGCTGATCAAGTTCGCCAGCCAGAGCCTGAGCGGCGTGCCCAGCAGGTTCTCTGGCTCTGGCAGCGGCAC |
| | CGACTTCACCTTAACCATCAACAGCCTGGAGGCCGAAGACGCCGCCACCTACTACTGCCACCAGAGCAGGAGCCTGCCCTTTACCTTCGGCCCCGGCACCAA |
| | GGTTGACATCAACGCCGCCGCCATCGAGGTGATGTACCCTCCCCCTTACCTGGACAACGAGAAGAGCAACGGCACCATCATCCACGTGAAGGGCAAGCAC |
| | CTGTGCCCCAGCCCCCTGTTCCCCGGCCCCTCTAAGCCCTTCTGGGTTCTGGTGGTGGTGGGCGGCGTGCTGGCCTGCTACAGCCTGCTGGTGACCGTGGC |
| | CTTCATCATCTTCTGGGTGAGGAGTAAGAGGAGCAGGCTGCTGCACAGCGACTACATGAACATGACCCCCAGGCGACCCGGCCCCACCAGGAAGCACTAC |
| | CAGCCCTACGCCCCTCCAAGGGACTTCGCCGCCTACAGGAGCAGGGTGAAGTTCAGCAGGAGCGCCGACGCCCCCGCCTACCAGCAGGGCCAGAACCAGC |
| | TGTACAATGAGCTGAACCTGGGCAGGAGGGAGGAGTACGACGTGCTGGACAAGAGGAGGGGAAGGGACCCCGAGATGGGCGGCAAGCCCAGGAGGAA |
| | GAACCCCAGGAGGGCCTGTACAACGAGCTCCAGAAGGACAAAATGGCCGAAGCCTACAGCGAGATCGGCATGAAGGGCGAGAGGAGGAGGGGCAAA |
| | GGCCACGACGGCCTGTACCAGGGCCTGAGCACCGCCACCAAAGACACCTACGACGCCCTGCACATGCAGGCCCTGCCCCCTAGGTAATGATGA |
| 55 | ATGGACATGAGGGTGCCCGCCCAGCTGCTGGGCCTGCTGTTACTTTGGCTGCCCGGCGCCAAGTGCCAAGTGACCCTGAGGGAGTCTGGCCCCGCCCTGG |
| | TGAAGCCCACCCAGACCCTCACCCTGACCTGCACCTTCAGCGGCATCAGCCTGACCACCAGCGGCATGTGCGTGAGCTGGATCAGGCAGCCCCCTGGCAAG |
| | GCCCTGGAGTGGCTGGCCCTGATCGACTGGGACGGCGACAACTACTACAGCACCAGCCTGAAGACCAGGCTGACCATCTCCAAGGACACCAGCAAGAACC |
| | AGGTGGTGCTGACCATGGCCAGCATGGACCCCGTGGACACCGCCACATACTACTGCGCCAGGATGGGCTACACCTACGGCTACGACGCTTTCGACATCTGG |
| | GGCCAGGGCACCATGGTGACCGTGAGCAGCGGCGGAGGAGGATCTGGAGGCGGAGGCTCTGGTGGAGGAGGCAGCGGGGGGGGAGGAAGCGAGATT |
| | GTGCTGACACAGACCCCCAGCTCCCTGAGCGCCAGCCTGGGCGACAGGGTGACCATCAGCTGCAGGGCCTCCCAGAGCATCGGCAGCCTGTTGCACTGGT |
| | ACCAGCAGAAGCCCGATCAGAGCCCCAAACTGCTGATCAAGTTCGCCAGCCAGAGCCTGAGCGGCGTGCCCAGCAGGTTCTCTGGAAGCGGCTCTGGCAC |
| | CGACTTCACCTTAACCATCAACAGCCTGGAGGCCGAGGACGCCGCCACCTACTACTGCCACCAAAGCAGGAGCCTGCCCTTTACCTTCGGCCCCGGCACCAA |
| | GGTGGACATCAACGCCAGCAGCCTCAGGCCCGAAGCTTGTAGGCCTGCTGCCGGCGGCGCTGTTCACACCAGGGGCTTAGACTTTGCTTGCGACATCTACA |
| | TCTGGGCCCCCTTAGCCGGAACCTGCGGCGTGCTGCTGCTGAGCCTGGTGATCACCCTGTACTGCAACCAGGAATGTGAGGAGCAAGAGGAGCAGGCT |
| | GCTGCACAGCGACTACATGAACATGACCCCCAGGCGACCCGGCCCCACCAGGAAGCACTACCAGCCCTACGCCCCACCCAGGGACTTCGCCGCCTACAGGA |
| | GCAGGGTGAAGTTCAGCAGGAGCGCCGACGCCCCCGCCTACCAGCAGGGCCAGAATCAGCTGTACAATGAGCTGAACCTGGGCAGGAGGGAGGAGTAC |
| | GACGTGCTGGACAAGAGGAGGGGAAGGGACCCTGAGATGGGCGGCAAGCCCAGGAGGAAGAACCCCCAGGAGGGCCTGTACAACGAGCTCCAGAAGG |
| | ACAAGATGGCCGAGGCCTACAGCGAGATCGGCATGAAGGGCGAGAGGAGGAGGGGCAAGGGCCACGACGGCCTGTACCAGGGCTTAAGCACCGCCACC |
| | AAGGATACCTACGACGCCCTGCACATGCAGGCCCTGCCCCCTAGGTAATGATGA |
| 56 | ATGGACATGAGGGTGCCCGCCCAGCTTCTGGGCCTGCTGCTCTTGTGGCTGCCTGGCGCCAAGTGCCAGGTGACCCTGAGGGAGTCTGGCCCCGCCCTGG |
| | TGAAGCCCACCCAGACCTTGACCCTGACCTGCACCTTCAGCGGCATCAGCCTGACCACCAGCGGCATGTGCGTGAGCTGGATCAGGCAGCCCCCTGGCAAG |
| | GCCCTGGAGTGGCTGGCCCTGATCGACTGGGACGGCGACAACTACTACAGCACCAGCCTGAAGACCAGGCTGACCATCTCTAAGGACACCAGCAAGAACC |
| | AGGTGGTGCTGACCATGGCCAGCATGGACCCCGTGGACACCGCCACATACTACTGCGCCAGGATGGGCTACACCTACGGCTACGACGCTTTCGACATCTGG |
| | GGCCAGGGCACCATGGTGACCGTGAGCTCTGGCGGGGGAGGAAGCGGCGGCGGAGGATCTGGTGGAGGTGGGTCTGGAGGCGGCGGCAGCGAAATCG |
| | TGCTGACCCAGACCCCCAGCAGCCTCAGCGCCAGCCTGGGCGACAGGGTGACCATCAGCTGCAGGGCCTCCCAGAGCATCGGCAGCCTGTTGCACTGGTA |
| | CCAGCAGAAGCCCGACCAGAGCCCCAAGCTGCTGATCAAGTTCGCCAGCCAGAGCCTGAGCGGCGTGCCCAGCAGGTTCTCTGGAAGCGGCTCTGGAACC |
| | GACTTCACCTTAACCATCAACAGCCTGGAGGCCGAAGACGCCGCCACCTATTACTGCCACCAGAGCAGGAGCCTGCCCTTCACCTTCGGACCCGGAACCAA |
| | GGTGGACATCAACGCCAGCACAACCACACCQGCCCCTAGGCCCCCTACCCCCGCCCCCACAATCGCTAGCCAACCCCTGAGCCTTAGGCCCGAAGCCTGTA |
| | GACCTGCCGCCGGAGGCGCCGTGCATACCAGGGGCCTGGACTTCGCTTGTGACATCTACATCTGGGCCCCCTTAGCCGGAACCTGCGGCGTGCTGCTGCTG |
| | AGCCTGGTGATCACCCTGTACTGCAACCACAGGAACGTGAGGAGCAAAAGGAGCAGGCTGCTGCACAGCGACTACATGAACATGACCCCCAGGAGGCCC |
| | GGCCCCACCAGGAAGCACTACCAGCCCTACGCCCCACCCAGGGACTTCGCCGCCTACAGGAGCAGGGTGAAGTTTAGCAGGAGCGCCGACGCCCCCGCCT |
| | ACCAGCAGGGCCAGAATCAGCTGTACAATGAATTAAACCTGGGCAGGAGGGAGGAGTACGACGTGCTGGATAAGAGGAGGGGAAGGGACCCCGAGATG |
| | GGCGGCAAGCCCAGGAGGAAGAACCCTCAGGAGGGACTGTACAACGAGCTCCAGAAGGATAAGATGGCCGAGGCCTACAGCGAGATCGGCATGAAGGG |
| | CGAGAGGAGGAGGGGCAAGGGCCACGACGGCCTGTACCAAGGCCTGAGCACCGCCACCAAGGACACCTACGACGCCCTGCACATGCAGGCCCTGCCCCC |
| | TAGATAATGATGA |
| 57 | ATGGACATGAGGGTGCCCGCCCAACTGCTGGGCCTGCTGCTCCTTTGGCTGCCTGGCGCCAAGTGCCAGGTGACCCTGAGGGAGTCTGGCCCCGCCCTGG |
| | TGAAGCCCACCCAGACCTTGACCCTGACCTGCACCTTCAGCGGCATCAGCCTGACCACCAGCGGCATGTGCGTGAGCTGGATCAGGCAGCCCCCTGGCAAG |
| | GCCCTGGAGTGGCTGGCCCTGATCGACTGGGACGGCGACAACTACTACAGCACCAGCCTGAAGACCAGGCTGACCATCTCTAAGGACACCAGCAAGAACC |
| | AGGTGGTGCTGACCATGGCCAGCATGGACCCCGTGGACACCGCCACATACTACTGCGCCAGGATGGGCTACACCTACGGCTACGACGCTTTCGACATCTGG |
| | GGCCAGGGCACCATGGTGACCGTGAGCAGCGGCGGAGGCGGTTCTGGTGGCGGAGGCTCAGGAGGCGGAGGATCTGGAGGCGGCGGCAGCGAAATCG |
| | TGCTGACCCAGACCCCCAGCAGCCTCAGCGCCAGCCTCGGCGACAGGGTGACCATCAGCTGCAGGGCCTCCCAGAGCATCGGCAGCCTGCTGCACTGGTA |
| | CCAGCAGAAGCCCGACCAGAGCCCCAAGCTGCTGATCAAGTTCGCCAGCCAGAGCCTGAGCGGCGTGCCCAGCAGGTTCTCTGGCAGCGGCTCTGGCACC |
| | GACTTCACCTTAACCATCAACAGCCTGGAGGCCGAGGACGCCGCCACCTACTACTGCCATCAAAGCAGGAGCCTGCCCTTCACCTTCGGCCCCGGCACAAA |
| | AGTGGACATCAACGCCAGCCTGAGCAACAGCATCATGTACTTTAGCCATTTTGTGCCCGTGTTCCTGCCTGCCAAGCCCACAACAACCCCCGCCCCCAGGCC |
| | TCCTACCCCTGCCCCTACAATCGCTAGCCAACCCCTGTCTCTGAGACCCGAGGCCTGCAGACCTGCTGCCGGAGGCGCCGTGCACACCAGGGGACTGGACT |
| | TCGCTTGCGACATCTATATCTGGGCCCCTTAGCCGGCACCTGCGGCGTGCTGCTGCTGAGCCTGGTGATTACCCTGTACTGCAACCACAGGAACGTGAGG |
| | AGCAAGAGGAGCAGACTGCTGCACAGCGACTACATGAACATGACCCCCAGGCGACCCGGCCCCACCAGGAAGCACTACCAGCCCTACGCCCCACCCAGGG |
| | ACTTCGCCGCCTACAGGAGCAGGGTGAAGTTCAGCAGGTCAGCCGACGCCCCCGCCTACCAACAGGGCCAGAACCAGCTGTACAATGAGCTGAACCTGGG |
| | CAGGAGGGAGGAGTACGACGTGCTGGACAAGAGGAGGGGAAGGGATCCTGAGATGGGCGGCAAGCCCAGGAGGAAGAACCCCCAGGAGGGCCTGTAC |
| | AACGAGCTCCAGAAGGACAAGATGGCCGAGGCCTACAGCGAGATCGGCATGAAGGGCGAGAGGAGGAGGGGCAAGGGCCACGACGGCCTGTACCAGG |
| | GCCTGAGCACCGCTACCAAGGACACCTACGACGCCCTGCACATGCAGGCCCTGCCCCCTAGGTAATGATGA |
| 58 | ATGGACATGAGGGTGCCCGCCCAGCTGCTGGGCCTGTTACTGCGTGGCTGCCCGGCGCTAAGTGCCAGGTGACCCTGAGGGAGTCTGGCCCCGCCCTGG |
| | TGAAGACCACCCAGACCCTGACACTGACCTGCACCTTCAGCGGCTTCAGCCTGTCCACCAGCGGCATGTGCGTGAGCTGGATCAGGCAGCCCCCAGGCAAG |
| | GCCTTGGAGTGGCTGGCCCTGATCGACTGGGACGACGACAAGTACTACAGCACCAGCCTGAAGACCAGGCTGACCATCTCTAAGGACACCAGCAAGAGCC |
| | AGGTGGTGCTGACCATGACCAGCATGGACCCCGTGGACACCGCCACATACTACTGCGCCAGGATCAGGTACATCTACGGCTACGACTACTTCGACTACTGG |
| | GGCCAGGGAACCCTGGTCACCGTGAGCAGCGGAGGCGGTGGATCTGGAGGCGGAGGAAGCGGCGGAGGAGGATCCGGAGGCGGCGGCAGCGATATCC |
| | AGATGACCCAGAGCCCCAGCTCCCTGAGCGCCTCTGTGGGCGACAGGGTGACCATCACCTGCAGGGGCCTCTCAGAGGATCAGCTCTCAGCTGAACTGGTA |
| | CCAGCAGAAGCCCGGCAAGGCCCCCAAGCTGCTGATCTACGCCGCCAGCAGCCTCCAGAGCGGCGTGCCCTCTAGGTTCTCTGGCTCTGGAAGCGGCACC |
| | GAGTTCACCCTGACCATTAGCAGCTTGCAGCCCGAGGACATCGCCACCTACTGCTGCCAGCAGACCTACAGCAAGCCTATCACCTTCGGCCAGGGCACCAG |
| | GCTGGAGATCAAGGCCGCCGCCATCGAGGTGATGTACCCTCCCCCTTACCTGGACAACGAGAAGAGCAACGGCACCATCATCCACGTGAAGGGCAAGCAC |
| | CTGTGCCCAGCCCCCTGTTCCCCGGCCCCTCTAAGCCCTTCTGGGTCCTGGTGGTGGTGGGCGGCGTGCTGGCCTGCTACAGCCTGCTGGTGACCGTGGC |
| | CTTCATCATCTTCTGGGTGAGGAGCAAGAGGAGCAGGCTGCTGCACAGCGACTACATGAACATGACCCCCAGGCGACCCGGCCCCACCAGGAAGCACTAC |
| | CAGCCCTACGCCCCACCCAGGGACTTCGCCGCCTACAGGAGCAGGGTGAAGTTCAGCAGGAGCGCCGACGCCCCCGCCTACCAGCAGGGCCAGAACCAGC |
| | TGTACAATGAGCTGAACCTGGGCAGGAGGGAGGAGTACGACGTGCTGGACAAGAGGAGGGGAAGGGACCCCGAGATGGGCGGCAAGCCCAGGAGGAA |
| | GAACCCCCAGGAGGGCCTGTACAACGAGCTCCAGAAGGACAAGATGGCCGAGGCCTACAGCGAGATCGGCATGAAGGGCGAGAGGAGGAGGGGCAAG |
| | GGCCACGACGGCCTGTACCAGGGCCTGAGCACCGCCACCAAGGACACCTACGACGCCCTGCACATGCAGGCCTTACCCCCTAGGTAATGATGA |
| 59 | ATGGACATGAGGGTGCCCGCCCAACTGCTGGGCCTGTTGCTGCTTTGGCTGCCCGGCGCCAAGTGCCAGGTGACCCTGAGGGAGTCTGGACCCGCCCTGG |
| | TGAAGACCACCCAGACCCTGACACTGACCTGCACCTTCAGCGGCTTCAGCCTGTCCACCAGCGGCATGTGCGTGAGCTGGATCAGGCAGCCCCCTGGCAAG |
| | GCCTTGGAGTGGCTGGCCCTGATCGACTGGGACGACGACAAGTACTACAGCACCAGCCTGAAGACCAGGCTGACCATCAGCAAGGACACCAGCAAGAGC |
| | CAGGTGGTGCTGACCATGACCAGCATGGACCCCGTGGACACCGCCACATACTACTGCGCCAGGATCAGGTACATCTACGGCTACGACTACTTCGACTACTG |
| | GGGCCAGGGCACCCTGGTGACCGTGAGCAGCGGAGGCGGAGGATCTGGCGGAGGGGGATCTGGAGGTGGAGGCTCTGGCGGGGGGGGCAGCGATATC |
| | CAGATGACCCAGAGCCCCAGCTCCCTGAGCGCCAGCGTGGGCGACAGGGTGACCATCACCTGCAGGGCCTCCCAGAGGATCAGCTCCCAGCTGAACTGGT |
| | ACCAGCAGAAGCCCGGCAAGGCCCCCAAGCTGCTGATCTACGCCGCCAGCAGCCTTCAGAGCGGCGTGCCCAGCAGGTTCTCCGGCAGGGGGAGCGGCA |
| | CCGAGTTCACCCTGACCATTAGCTCACTGCAGCCCGAGGACATCGCCACCTACTGCTGCCAGCAGACCTACAGCAAGCCCATCACCTTCGGGCAGGGCACC |
| | AGGCTGGAGATCAAGGCCTCTTCTTTGAGACCCGAGGCTTGTAGGCCCGCAGCCGGCGGCGCCGTGCACACAAGGGGACTGGATTTCGCTTGCGACATCT |
| | ATATCTGGGCCCCCTTAGCCGGCACCTGCGGCGTGCTGCTGCTGAGCCTGGTGATCACCCTGTACTGCAACCACAGGAACGTGAGGAGCAAGAGGAGCAG |
| | GCTGCTGCACAGCGACTACATGAACATGACACCCAGGAGGCCCGGCCCTACCAGGAAGCACTACCAGCCCTACGCCCCACCCAGGGACTTCGCCGCTTATA |
| | GGAGCAGGGTGAAGTTCAGCAGGAGCGCCGACGCTCCCGCCTACCAGCAGGGCCAGAACCAGCTGTACAATGAGCTGAACCTGGGCAGGAGGGAGGAG |
| | TACGACGTGCTGGACAAGAGAAGGGGAAGGGACCCCGAGATGGGGGGCAAGCCCAGGAGGAAGAACCCCCAGGAGGGCCTGTACAACGAGCTCCAGAA |
| | GGACAAGATGGCCGAGGCCTACAGCGAGATCGGCATGAAGGGCGAGAGGAGGAGGGGCAAGGGCCACGACGGCCTGTACCAGGGCCTGAGCACCGCC |
| | ACCAAAGACACCTACGACGCCCTGCACATGCAGGCCCTGCCCCCTAGGTAATGATGA |
| 60 | ATGGACATGAGGGTGCCCGCCCAGCTGTTGGGCCTGCTGCTGCTTTGGCTGCCTGGCGCCAAGTGCCAGGTGACCCTGAGGGAGTCTGGCCCCGCCCTGG |
| | TGAAGACCACCCAGACCCTGACACTGACCTGCACCTTCAGCGGCTTCAGCCTGTCCACCAGCGGCATGTGCGTGAGCTGGATCAGGCAGCCCCCTGGAAAG |
| | GCCCTGGAGTGGCTGGCCCTGATCGACTGGGACGACGACAAGTACTACAGCACCAGCCTGAAGACCAGGCTGACCATCTCTAAGGACACCAGCAAGAGCC |
| | AGGTGGTGCTGACCATGACCAGCATGGACCCCGTGGACACCGCCACATACTACTGCGCCAGGATCAGGTACATCTACGGCTACGACTACTTCGACTACTGG |
| | GGCCAGGGCACCCTGGTGACCGTGAGCTCTGGCGGCGGAGGAAGCGGAGGTGGCGGAAGCGGTGGAGGAGGATCTGGGGGGGGCGGCAGCGATATCC |
| | AGATGACCCAGAGCCCCAGCTCCCTGAGCGCCAGCGTGGGCGACAGGGTGACCATCACCTGCAGGGCCTCCCAGAGGATCAGCAGCCAGCTGAACTGGTA |
| | CCAGCAGAAGCCCGGCAAGGCCCCTAAGCTCCTGATCTACGCCGCCAGCAGCCTCCAGAGCGGCGTGCCCAGCAGGTTCTCCGGCTCTGGAAGCGGAACC |
| | GAGTTCACCTTGACCATCAGTAGCCTGCAGCCCGAGGATATCGCCACCTACTGCTGCCAGCAGACCTACAGCAAACCCATCACCTTCGGCCAAGGCACCAG |
| | GCTGGAGATCAAGGCCAGCACCACAACCCCTGCCCCTAGGCCCCCTACACCCGCTCCCACCATCGCTAGCCAACCTCTGTCTCTGAGACCTGAGGCTTGCAG |
| | ACCTGCTGCCGGCGGCGCCGTGCACACCAGAGGCCTGGACTTCGCTTGCGACATCTACATTTGGGCCCCCTTAGCCGGAACCTGCGGCGTTCTGCTGCTGA |
| | GCCTGGTGATCACCCTGTACTGCAACCACAGGAACGTGAGGAGCAAGAGGAGCAGGCTGCTGCATAGCGACTACATGAACATGACCCCCAGGCGACCCG |
| | GCCCCACCAGGAAGCACTACCAGCCCTATGCCCCACCCAGGGACTTCGCCGCTTACAGGAGCAGGGTGAAGTTCAGCAGGAGCGCCGACGCCCCCGCCTA |
| | CCAGCAGGGCCAGAACCAGCTGTACAATGAGCTGAACCTGGGCAGGAGGGAGGAGTACGACGTGCTGGACAAGAGGAGGGGAAGGGACCCCGAGATG |
| | GGCGGCAAGCCCAGGAGGAAGAACCCCCAGGAGGGCCTGTACAACGAGCTCCAGAAGGACAAGATGGCCGAGGCCTACAGCGAGATCGGCATGAAGGG |
| | CGAGAGGAGGAGGGGCAAGGGCCACGACGGCCTGTACCAGGGCCTGAGCACCGCCACCAAGGACACCTACGACGCCCTGCACATGCAGGCCCTGCCCCC |
| | TAGGTAATGATGA |
| 61 | ATGGACATGAGGGTGCCCGCCCAGCTGCTGGGCCTGCTTCTGTTATGGCTGCCCGGCGCCAAGTGCCAGGTGACCCTGAGGGAGTCTGGACCCGCCCTGG |
| | TGAAGACCACCCAGACCCTGACACTGACCTGCACCTTCAGCGGCTTCAGCCTGTCCACCAGCGGCATGTGCGTGAGCTGGATCAGGCAGCCCCCTGGAAAG |
| | GCCCTGGAGTGGCTGGCCCTGATCGACTGGGACGACGACAAGTACTACAGCACCAGCCTGAAGACCAGGCTGACCATCTCTAAGGACACCAGCAAGAGCC |
| | AGGTGGTGCTGACCATGACCAGCATGGACCCCGTGGACACCGCCACATACTACTGCGCCAGGATCAGGTACATCTACGGCTACGACTACTTCGACTACTGG |
| | GGCCAGGGCACCCTGGTGACCGTGAGCAGCGGTGGAGGAGGCTCTGGCGGAGGAGGGTCCGGAGGAGGTGGATCTGGAGGCGGCGGCAGCGACATCC |
| | AGATGACCCAGAGCCCCAGCTCCCTGAGCGCCAGCGTGGGCGACAGGGTGACCATCACCTGCAGGGCCTCCCAGAGGATCAGCTCCCAGCTGAACTGGTA |
| | CCAGCAGAAGCCCGGCAAGGCCCCCAAGCTGCTGATCTACGCCGCCAGCAGCCTCCAGAGCGGCGTGCCCAGCAGGTTCTCCGGAAGCGGCAGCGGCACC |
| | GAGTTCACCCTGACCATTTCCAGCCTGCAGCCCGAGGACATCGCCACCTACTGCTGCCAGCAGACCTACAGCAAGCCCATCACCTTCGGCCAAGGCACCAG |
| | GCTGGAGATCAAGGCCAGCCTGAGCAACAGCATCATGTACTTCAGCCACTTCGTGCCCGTGTTCCTGCCCGCCAAGCCCACAACCACCCCTGCTCCCAGACC |
| | CCCTACACCCGCCCCCACCATCGCCAGCCAACCCCTTTCTTTGAGACCCGAGGCCTGCAGACCCGCCGCCGGCGGAGCTGTGCATACAAGGGGCTTGGACT |
| | TCGCTTGCGACATCTACATCTGGGCCCCCTTAGCCGGCACCTGCGGCGTGCTGCTGCTGAGCCTGGTGATCACCCTGTACTGCAACCACAGGAACGTGAGG |
| | AGCAAGAGGAGCAGGCTGCTGCACAGCGACTACATGAACATGACCCCCAGGAGGCCCGGCCCCACCAGGAAGCACTACCAGCCCTATGCTCCACCCAGGG |
| | ATTTTGCCGCCTACAGGAGCAGGGTGAAGTTCAGCAGGAGCGCCGACGCCCCCGCCTACCAGCAGGGCCAGAACCAGCTGTACAATGAGCTGAACCTGGG |
| | CAGGAGGGAGGAGTATGACGTGCTGGACAAGAGGAGGGGAAGGGACCCCGAGATGGGCGGCAAGCCCAGGAGGAAGAACCCTCAGGAGGGCCTGTAC |
| | AACGAGCTCCAGAAGGACAAGATGGCCGAGGCCTACAGCGAGATCGGCATGAAGGGCGAGAGGAGGAGGGGCAAGGGCCACGACGGCTTGTACCAGG |
| | GCCTGAGCACCGCCACCAAGGACACCTACGACGCCCTGCACATGCAGGCCCTGCCCCCTAGGTAATGATAA |
| 62 | ATGGACATGAGGGTGCCCGCCCAGCTGCTGGGACTTCTGCTGCTGTGGCTTCCCGGCGCCAAGTGCCAGGTGCAGCTGCAGCAGAGCGGCCCCGGACTGG |
| | TGAAGCCCAGCCAGACCCTGAGCCTGACCTGCGCCATCAGCGGCGACAGCGTGAGCAGCAACAGCGCCGCCTGGAACTGGATCAGGCAGAGCCCCAGCA |
| | GGGGCCTGGAGTGGCTGGGCAGAACCTACTACAGGTCCAAGTGGTACAACGACTACGCCGTGAGCGTGAAGAGCAGGATCACCATCAACCCCGACACCA |
| | GCAAGAACCAGTTCTCACTGCAGCTGAACAGCGTGACCCCCGAGGACACCGCCGTGTACTACTGCGCCAGGGAGGTGACCGGCGACCTGGAGGACGCCTT |
| | CGACATCTGGGGCCAGGGAACCATGGTGACAGTGAGCAGCGGCGGAGGCGGCTCAGGAGGCGGCGGATCTGGTGGAGGAGGAAGTGGTGGAGGTGGC |
| | AGCGACATCCAGATGACACAGAGCCCTTCTAGCCTGAGCGCCAGCGTGGGCGACAGGGTGACCATCACATGCAGGGCCTCTCAGACCATCTGGAGCTATC |
| | TGAACTGGTACCAGCAGAGGCCCGGAAAGGCCCCCAACCTGCTGATCTACGCCGCCAGCAGCCTCCAGAGCGGCGTGCCCAGCAGGTTCTCTGGCAGGGG |
| | CAGCGGCACCGACTTCACCTTAACCATCAGCAGCCTTCAGGCCGAGGACTTCGCTACCTACTACTGCCAGCAGAGCTACAGCATCCCCCAGACCTTCGGCCA |
| | GGGCACCAAGCTGGAGATCAAGGCCGCCGCCATCGAGGTGATGTACCCTCCCCCTTACCTGGACAACGAGAAGTCTAACGGCACCATCATCCACGTGAAG |
| | GGCAAGCACCTGTGCCCCAGCCCCCTGTTCCCCGGCCCCTCTAAGCCCTTCTGGGTCCTGGTGGTGGTGGGCGGCGTGCTGGCCTGCTACAGCCTGCTGGT |
| | GACCGTGGCCTTCATCATCTTCTGGGTGAGGAGCAAGAGGAGCAGGCTGCTGCACAGCGACTACATGAATATGACCCCCAGGCGACCCGGCCCCACCAGG |
| | AAACACTACCAGCCCTACGCCCCACCCAGGGACTTCGCCGCCTACAGGAGCAGGGTGAAATTCAGCAGGAGCGCCGACGCCCCCGCCTACCAGCAGGGCC |
| | AGAACCAGCTGTACAATGAGCTGAACCTGGGCAGGAGGGAGGAGTACGACGTGCTGGACAAGAGGAGGGGAAGGGACCCCGAGATGGGCGGCAAGCC |
| | CAGGAGGAAGAACCCCCAGGAGGGCCTGTACAACGAGCTCCAGAAGGACAAGATGGCCGAGGCCTACAGCGAGATCGGCATGAAGGGCGAGAGGAGG |
| | AGGGGCAAGGGCCACGACGGCCTGTACCAGGGCCTGAGCACCGCCACCAAGGACACCTACGACGCCCTGCACATGCAAGCTCTGCCCCCTAGGTAATGAT |
| | GA |
| 63 | ATGGACATGAGGGTTCCCGCCCAGCTGCTGGGCCTGCTGCTCCTTTGGCTGCCCGGAGCCAAGTGCCAGGTGCAGCTGCAGCAGAGCGGACCCGGCCTGG |
| | TGAAGCCCAGCCAGACCCTGAGCCTGACCTGCGCCATCAGCGGCGACAGCGTGAGCAGCAACAGCGCCGCCTGGAACTGGATCAGGCAGAGCCCCAGCA |
| | GGGGCCTGGAGTGGCTGGGCAGAACCTACTACAGGTCCAAGTGGTACAACGACTACGCCGTGAGCGTGAAGAGCAGGATCACCATCAACCCCGACACCA |
| | GCAAGAACCAGTTCTCACTGCAGCTGAACAGCGTGACCCCCGAGGACACCGCCGTGTACTACTGCGCCAGGGAGGTGACCGGCGACCTGGAGGACGCCTT |
| | CGACATCTGGGGCCAGGGAACCATGGTGACCGTGAGCTCCGGAGGGGGAGGATCTGGAGGTGGCGGAAGCGGCGGAGGTGGCTCTGGCGGAGGCGGC |
| | AGCGATATCCAGATGACCCAGAGCCCCTCCAGCCTGAGCGCCAGCGTGGGCGACAGGGTGACCATCACCTGCAGGGCCTCCCAGACCATCTGGAGCTACC |
| | TGAACTGGTACCAGCAGAGGCCCGGCAAGGCCCCCAACCTGCTGATCTACGCCGCCAGCAGCTTACAAAGCGGCGTGCCCAGCAGGTTCTCTGGCAGGGG |
| | CAGCGGCACCGACTTCACCTTAACCATCAGCAGCCTGCAGGCCGAGGACTTCGCTACCTACTACTGCCAGCAGAGCTACAGCATCCCCCAGACCTTCGGCCA |
| | GGGCACCAAGCTGGAAATCAAAGCTAGCAGCCTTAGACCCGAGGCCTGCAGACCCGCCGCCGGCGGAGCTGTGCATACAAGGGGCCTTGATTTTGCTTGC |
| | GACATCTACATCTGGGCCCCCTTAGCCGGCACCTGCGGCGTGCTGCTGCTGAGCCTGGTGATCACCCTGTACTGCAACCACAGGAACGTGAGGAGCAAGA |
| | GGAGCAGGCTTCTGCACAGCGACTACATGAATATGACCCCCAGGCGACCCGGCCCCACCAGGAAGCACTATCAGCCCTACGCCCCACCCAGGGACTTCGCC |
| | GCCTACAGGAGCAGGGTGAAGTTCAGCAGGAGCGCCGACGCCCCCGCCTACCAGCAGGGCCAGAACCAGCTGTACAATGAGCTGAACCTTGGCAGGAGG |
| | GAGGAGTACGACGTGCTTGATAAGAGGAGGGGAAGGGACCCCGAGATGGGGGGCAAGCCCAGGAGGAAGAACCCCCAGGAGGGCCTGTACAACGAGC |
| | TCCAGAAGGACAAGATGGCCGAGGCCTACTCTGAGATCGGCATGAAGGGCGAGAGGAGGAGGGGCAAGGGCCACGATGGCCTGTACCAGGGCCTGAGC |
| | ACCGCCACCAAGGACACCTACGACGCCCTGCACATGCAGGCCCTGCCCCCTAGGTAATGATGA |
| 64 | ATGGACATGAGGGTGCCCGCCCAGCTGCTTGGCCTGTTGCTGCTTTGGCTGCCCGGCGCCAAGTGCCAAGTGCAGCTGCAGCAGAGCGGACCCGGCCTGG |
| | TGAAGCCCAGCCAGACCCTGAGCCTGACCTGCGCCATCAGCGGCGACAGCGTGAGCTCCAACAGCGCCGCCTGGAACTGGATCAGGCAGAGCCCCAGCAG |
| | GGGCCTGGAGTGGCTGGGCAGAACCTACTACAGGTCCAAGTGGTACAACGACTACGCCGTGAGCGTGAAGAGCAGGATCACCATCAACCCCGACACCAG |
| | CAAGAACCAGTTCTCACTGCAGCTGAACAGCGTGACCCCCGAGGACACCGCCGTGTACTACTGCGCCAGGGAGGTGACCGGCGACCTGGAGGACGCCTTC |
| | GACATCTGGGGCCAGGGAACCATGGTGACCGTGAGCAGCGGCGGAGGTGGCTCTGGCGGAGGAGGATCTGGTGGGGGAGGAAGCGGCGGTGGCGGCA |
| | GCGACATCCAGATGACCCAAAGCCCCAGCTCCCTTAGCGCCAGCGTGGGCGACAGGGTGACCATCACCTGCAGGGCCTCTCAGACCATCTGGAGCTATCTG |
| | AACTGGTACCAGCAGAGGCCCGGCAAAGCTCCCAACCTGTTGATCTACGCCGCCAGCAGCCTCCAGAGCGGCGTGCCTAGCAGGTTTAGTGGAAGGGGCA |
| | GCGGCACCGACTTCACCTTAACCATCAGCAGCCTTCAAGCCGAGGACTTTGCCACCTACTACTGCCAGCAGAGCTACAGCATCCCCCAGACCTTCGGCCAGG |
| | GCACCAAGCTGGAGATCAAGGCTAGCACCACAACACCCGCCCCCAGGCCCCCTACACCCGCTCCCACAATCGCCTCTCAGCCTCTGAGCCTTAGGCCCGAA |
| | GCTTGCAGACCTGCTGCCGGCGGCGCCGTGCATACCAGGGGACTGGACTTCGCTTGCGACATCTACATCTGGGCCCCCTTAGCCGGCACATGCGGCGTGCT |
| | GCTGCTGAGCCTGGTGATCACCCTGTACTGCAACCACAGGAACGTGAGGAGCAAGAGGAGCAGGCTGCTGCACAGCGACTACATGAACATGACCCCCAGG |
| | CGACCCGGCCCCACCAGGAAGCACTACCAGCCCTACGCCCCACCCAGGGACTTCGCCGCCTACAGGAGCAGGGTGAAGTTCAGCAGGAGCGCCGACGCCC |
| | CCGCCTACCAGCAGGGCCAGAACCAGCTGTACAATGAACTGAACCTGGGCAGGAGGGAGGAATACGACGTTCTGGACAAGAGAAGGGGAAGGGACCCC |
| | GAGATGGGCGGCAAGCCCAGGAGGAAGAACCCCCAGGAGGGCCTGTACAACGAGCTCCAGAAGGACAAGATGGCCGAGGCCTACAGCGAGATCGGCAT |
| | GAAGGGCGAGAGGAGGAGGGGCAAGGGCCACGACGGCCTGTACCAGGGCCTGAGCACCGCCACCAAGGACACCTATGACGCCCTGCACATGCAGGCCC |
| | TGCCCCCTAGGTAATGATGA |
| 65 | ATGGACATGAGGGTGCCCGCCCAGCTGCTTGGCCTGCTGCTCCTTTGGCTGCCTGGCGCCAAGTGCCAGGTGCAGCTGCAGCAGAGCGGACCCGGCCTGG |
| | TGAAGCCCAGCCAGACCCTGAGCCTGACCTGCGCCATCAGCGGCGACAGCGTGAGCAGCAACAGCGCCGCCTGGAACTGGATCAGGCAGAGCCCCAGCA |
| | GGGGCCTGGAGTGGCTGGGCAGAACCTACTACAGGTCCAAGTGGTACAACGACTACGCCGTGAGCGTGAAGAGCAGGATCACCATCAACCCCGACACCA |
| | GCAAGAACCAGTTCTCACTGCAGCTGAACAGCGTGACCCCCGAGGACACCGCCGTGTACTACTGCGCCAGGGAGGTGACCGGCGACCTGGAGGACGCCTT |
| | CGACATCTGGGGCCAGGGCACCATGGTGACCGTGAGCTCCGGGGGGGGAGGATCTGGAGGGGGAGGTTCTGGTGGGGGGGGAAGCGGAGGCGGCGGA |
| | AGCGACATCCAGATGACCCAGAGCCCTAGCAGCCTGAGCGCCAGCGTGGGCGACAGGGTGACCATCACCTGCAGGGCCTCCCAGACCATCTGGAGCTACC |
| | TGAACTGGTACCAGCAGAGGCCCGGCAAAGCTCCCAACTTATTGATCTACGCCGCCAGCAGCCTCCAGAGCGGCGTGCCCAGCAGGTTCTCTGGCAGGGG |
| | CAGCGGCACCGACTTCACCTTAACCATCAGCAGCCTTCAGGCCGAGGACTTCGCTACCTACTACTGCCAGCAGAGCTACAGCATCCCCCAGACCTTCGGCCA |
| | AGGCACCAAGCTGGAGATCAAGGCCAGCCTTAGCAACAGCATCATGTACTTCAGCCACTTCGTGCCCGTTTTCCTGCCTGCCAAACCCACCACAACCCCTGC |
| | TCCCAGGCCCCCTACCCCCGCTCCTACCATTGCTAGCCAACCTCTGAGCCTTAGGCCCGAGGCCTGCAGACCCGCTGCCGGAGGCGCTGTGCACACAAGGG |
| | GACTGGACTTCGCATGCGACATCTACATCTGGGCCCCCTTAGCCGGCACCTGCGGCGTGCTGCTGCTGAGCCTGGTGATCACCCTGTACTGCAACCACAGG |
| | AACGTGAGGAGCAAGAGGAGCAGGCTGCTGCACAGCGACTACATGAACATGACCCCCAGGCGACCCGGCCCCACCAGGAAGCACTACCAGCCCTACGCC |
| | CCACCCAGGGACTTCGCCGCCTACAGGAGCAGGGTGAAGTTCAGCAGGAGCGCCGACGCCCCCGCCTACCAGCAGGGACAGAACCAGCTGTACAATGAG |
| | CTGAACCTGGGCAGGAGGGAGGAGTACGACGTGCTGGACAAGAGGAGGGGAAGGGATCCCGAGATGGGGGGCAAGCCCAGGAGGAAGAACCCCCAGG |
| | AGGGCCTGTACAACGAGCTCCAGAAGGACAAGATGGCCGAGGCCTACAGCGAAATCGGCATGAAGGGCGAGAGGAGGAGGGGCAAGGGCCACGACGG |
| | CCTGTACCAGGGCCTGAGCACCGCCACCAAGGACACATACGACGCCCTGCACATGCAGGCCCTGCCCCCTAGGTAATGATAA |
| 66 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTQCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATQCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGGGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTCCTGGGCCTGCTGTTACTGTGGCTGCCCGGCGCTAAGTGCCAGGTACAGCTCGTGGAGAGCGGCGGAGGCGTGGTGCAGCCCGGCAGGAGCCTG |
| | AGGTTAAGCTGCGCCGCCAGCGGCTTCACCTTCAGCAGCTACGGCATGCACTGGGTGAGACAGGCCCCCGGCAAGGGCTTGGAGTGGGTGGCCATCATCT |
| | ACTACGACGGCAGCAAGAAGTACTACGCCGACAGCGTGAAGGGAAGGTTCACCATCAGCAGGGACAACAGCAAGAACACCCTGTACCTGCAGATGAACA |
| | GCCTGAGGGCCGAGGACACCGCCGTGTACTACTGCGCCAGGGAGCTGACCGGCGACGCCTTCGACATCTGGGGCCAGGGAACCAAGGTGACCGTCAGCA |
| | GCGGGGGGGGAGGATCTGGCGGAGGAGGATCAGGAGGCGGCGGATCTGGGGGGGGCGGAAGCGAGATCGTGCTGACCCAGACCCCCAGCAGCCTGAG |
| | CGCCAGCCTGGGCGACAGGGTGACCATCAGCTGCAGGGCCTCCCAGAGCATCGGCTCCAGCCTGCACTGGTACCAGCAGAAGCCCGACCAGAGCCCCAAG |
| | CTGCTGATCAAGTACGCCAGCCAGAGCTTCAGCGGCGTGCCCAGCAAGTTCATCGGCAGCGGAAGCGGCACCGACTTCACCTTGACCATCAACAGCCTGG |
| | AGGCCGAAGACGCCGCCACCTTTTATTGCCACCAGAGCAGCACCCTGCCCTACACCTTCGGCCAGGGCACCAAGCTGGAGATCAAGGCCGCCGCCATCGAG |
| | GTGATGTACCCTCCCCCTTACCTTGACAACGAGAAGAGCAACGGCACCATCATCCACGTGAAGGGCAAGCACCTGTGCCCCAGCCCCCTGTTCCCCGGCCCC |
| | TCTAAGCCCTTCTGGGTCCTGGTGGTGGTTGGCGGCGTGCTGGCCTGCTACAGCCTGCTGGTGACCGTGGCCTTCATCATCTTCTGGGTGAGGAGCAAAAG |
| | GAGCAGGCTGCTTCACAGCGACTACATGAACATGACCCCCAGGCGACCCGGCCCCACCAGGAAGCACTACCAGCCCTATGCCCCACCCAGGGACTTQGCCG |
| | CCTACAGGTCTAGGGTGAAGTTCAGCAGGAGCGCCGACGCCCCCGCCTACCAGCAGGGCCAGAACCAGCTGTACAATGAACTGAACCTGGGCAGGAGGG |
| | AGGAGTACGACGTGCTGGACAAGAGGAGGGGAAGGGACCCCGAGATGGGGGGCAAGCCCAGGAGGAAGAACCCCCAGGAAGGCCTGTACAACGAGCT |
| | CCAGAAGGATAAAATGGCCGAGGCCTACAGCGAAATCGGCATGAAGGGCGAGAGGAGGAGGGGCAAGGGCCACGACGGCCTTTACCAGGGCCTGAGCA |
| | CCGCCACCAAGGACACCTACGACGCCCTGCACATGCAGGCCCTGCCCCCTAGGTAATGATGA |
| 67 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGQGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGCCTGTTACTCCTTTGGCTGCCCGGCGCCAAGTGCCAGGTGCAGCTGGTTGAGAGCGGAGGCGGCGTGGTGCAGCCCGGCAGGAGCCTG |
| | AGGTTAAGCTGCGCCGCCAGCGGCTTCACCTTCAGCAGCTACGGCATGCACTGGGTGAGGCAGGCCCCCGGCAAGGGCTTGGAGTGGGTGGCCATCATCT |
| | ACTACGACGGCAGCAAGAAGTACTACGCCGACAGCGTGAAGGGCAGGTTCACCATCAGCAGGGACAACAGCAAGAACACCCTGTACCTGCAGATGAACA |
| | GCCTGAGAGCCGAGGACACCGCCGTGTACTACTGCGCCAGGGAGCTGACCGGCGACGCCTTCGACATCTGGGGCCAGGGAACCAAGGTGACCGTGAGCA |
| | GCGGCGGCGGAGGATCTGGCGGAGGAGGCTCAGGAGGAGGTGGAAGCGGGGGGGGCGGCTCTGAGATCGTGCTGACCCAGACCCCCAGCTCTCTGAGC |
| | GCCAGCCTGGGCGACAGGGTGACCATCAGCTGCAGGGCCTCCCAGAGCATCGGCTCTAGCCTGCACTGGTACCAGCAGAAGCCCGACCAGAGCCCCAAGC |
| | TGCTGATCAAGTACGCCAGCCAGAGCTTCAGCGGCGTGCCCAGCAAGTTCATCGGCAGCGGGAGCGGCACCGACTTCACCTTGACCATCAACAGCCTGGA |
| | GGCCGAGGACGCCGCCACCTTCTACTGCCACCAGAGCAGCACCCTGCCCTACACCTTCGGCCAGGGCACCAAGCTGGAGATCAAGGCCAGCAGCTTAAGA |
| | CCCGAAGCCTGCAGGCCCGCCGCCGGAGGAGCTGTTCACACCAGGGGCTTGGATTTTGCTTGCGATATCTACATCTGGGCCCCCTTAGCCGGCACCTGCGG |
| | CGTGCTGCTGCTGTCTCTGGTGATCACCTTATACTGTAACCACAGGAACGTGAGGAGCAAGAGAAGCAGGCTGCTGCACAGCGACTACATGAACATGACCC |
| | CTAGGAGGCCCGGCCCCACCAGGAAGCACTACCAGCCCTACGCCCCACCCAGGGACTTCGCCGCCTACAGGAGCAGGGTGAAGTTCAGCAGGAGCGCCG |
| | ACGCCCCCGCCTACCAGCAGGGCCAGAACCAGCTGTACAATGAGCTGAACCTGGGCAGGAGGGAGGAGTACGACGTGCTTGACAAGAGGAGGGGAAGG |
| | GACCCCGAAATGGGGGCAAGCCCAGGAGGAAAAACCCCCAAGAGGGCCTGTACAACGAGCTCCAGAAGGACAAGATGGCCGAGGCCTACAGCGAGAT |
| | CGGCATGAAGGGCGAGAGGAGGAGGGGCAAGGGCCACGACGGCCTGTACCAGGGCCTTAGCACCGCCACCAAGGACACCTACGACGCCCTGCACATGC |
| | AGGCCCTGCCCCCTAGGTAATGATGA |
| 68 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTCCCATAGTA |
| | ACGCCAATAGGGACTTTCCATGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGACTGCTGCTCCTTTGGCTGCCTGGCGCCAAGTGCCAGGTGCAGCTGGTGGAGAGCGGCGGAGGAGTGGTGCAGCCCGGCAGGAGCCTG |
| | AGACTGAGCTGCGCCGCCAGCGGCTTCACCTTCAGCAGCTACGGCATGCACTGGGTGAGGCAGGCCCCCGGCAAGGGCTTGGAGTGGGTGGCCATCATCT |
| | ACTACGACGGCAGCAAGAAGTACTACGCCGACAGCGTGAAGGGCAGGTTCACCATCAGCAGGGACAACAGCAAGAACACCCTGTATCTGCAGATGAACA |
| | GCCTGAGGGCCGAGGATACCGCCGTGTACTACTGCGCCAGGGAGCTGACCGGCGACGCCTTCGACATCTGGGGCCAGGGAACCAAGGTGACCGTGAGCA |
| | GCGGAGGCGGAGGATCTGGAGGTGGCGGTAGCGGAGGAGGCGGAAGCGGCGGTGGCGGCAGCGAAATTGTGCTGACCCAGACCCCCAGCAGCCTCAG |
| | CGCCAGCCTGGGCGACAGGGTGACCATCAGCTGCAGGGCCTCCCAGAGCATCGGCTCCAGCCTGCACTGGTACCAGCAGAAGCCCGACCAGAGCCCCAAG |
| | CTGCTGATCAAGTACGCCAGCCAAAGCTTCAGCGGCGTGCCCAGCAAGTTCATCGGAAGCGGATCCGGAACCGACTTCACCTTAACCATCAACAGCCTGGA |
| | GGCCGAGGACGCCGCCACCTTCTACTGTCACCAGAGCAGCACCCTGCCCTACACCTTTGGCCAGGGCACCAAGCTGGAAATCAAAGCCAGCACCACCACCC |
| | CCGCTCCTAGGCCTCCCACACCCGCCCCCACAATTGCTAGCCAGCCTTTGAGCCTGAGGCCTGAAGCTTGTAGACCCGCTGCCGGAGGCGCCGTGCACACC |
| | AGGGGCCTGGACTTCGCTTGCGACATCTACATCTGGGCCCCCTTGGCCGGCACCTGCGGCGTGCTGCTGCTGAGCCTGGTGATCACCCTGTACTGCAACCA |
| | CAGGAACGTGAGGAGCAAGAGGAGCAGGCTGCTGCACAGCGACTACATGAACATGACCCCCAGGCGACCCGGCCCCACCAGGAAGCACTACCAGCCCTA |
| | CGCCCCACCCAGGGACTTCGCCGCCTATAGGAGCAGGGTGAAGTTCAGCAGGAGCGCCGACGCCCCCGCCTACCAGCAGGGCCAGAACCAGCTGTACAAT |
| | GAGCTGAACCTGGGCAGGAGGGAGGAGTACGACGTGTTAGACAAGAGGAGGGGAAGGGATCCCGAGATGGGCGGCAAGCCCAGGAGGAAGAACCCCC |
| | AGGAAGGCCTGTACAACGAGCTCCAGAAGGACAAGATGGCTGAGGCCTACAGCGAAATCGGAATGAAGGGCGAGAGGAGGAGGGGCAAGGGCCACGA |
| | CGGCCTGTATCAGGGCCTGAGCACCGCCACCAAGGACACCTACGACGCCCTGCACATGCAGGCCCTGCCCCCTAGGTAATGATGA |
| 69 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAACCCCTCAAGACCCGTTTAGAGGCCCCAAGGGGTTATGCTATCAATCGTTGCGTTACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTCCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAACTGCTTGGCCTGCTGCTCCTTTGGCTGCCCGGCGCCAAGTGCCAGGTGCAGCTGGTGGAGTCTGGCGGAGGCGTGGTGCAGCCCGGAAGGAGCCTG |
| | AGACTGAGCTGCGCCGCCAGCGGCTTCACCTTCAGCAGCTACGGCATGCACTGGGTGAGGCAGGCCCCCGGCAAGGGCTTGGAGTGGGTGGCCATCATCT |
| | ACTACGACGGCAGCAAGAAGTACTACGCCGACAGCGTGAAGGGCAGGTTCACCATCAGCAGGGACAACAGCAAGAACACCCTGTATCTGCAGATGAACA |
| | GCCTGAGGGCCGAGGATACCGCCGTGTACTACTGCGCCAGGGAGCTGACCGGCGACGCCTTCGACATCTGGGGCCAGGGAACCAAGGTGACCGTGAGCA |
| | GCGGCGGAGGAGGATCTGGAGGCGGCGGATCAGGAGGTGGGGGAAGCGGGGGAGGCGGCAGCGAAATCGTGCTGACCCAGACCCCCAGCAGCCTCAG |
| | CGCCAGCCTGGGCGACAGGGTGACCATCAGCTGCAGGGCCTCCCAGAGCATCGGCTCCAGCCTGCACTGGTACCAGCAGAAGCCCGACCAGAGCCCCAAG |
| | CTGCTGATCAAGTACGCCAGCCAAAGCTTCAGCGGCGTGCCCAGCAAGTTCATCGGCAGCGGAAGCGGCACCGACTTCACCTTGACCATCAACAGCCTGGA |
| | GGCCGAGGACGCCGCCACCTTCTACTGCCACCAGAGCAGCACCCTGCCCTACACCTTCGGCCAGGGCACCAAGCTGGAGATCAAGGCCAGCCTGAGCAAC |
| | AGCATCATGTACTTCAGCCACTTCGTGCCCGTGTTCCTGCCCGCCAAGCCTACCACAACACCCGCCCCCAGGCCCCCAACACCTGCTCCCACAATCGCCAGCC |
| | AGCCCTTATCCTTAAGGCCCGAGGCCTGCAGACCCGCTGCCGGAGGCGCTGTGCATACCAGGGGACTGGACTTCGCTTGCGACATCTACATCTGGGCCCCC |
| | TTAGCCGGAACATGCGGCGTGCTGCTGCTGAGCCTGGTGATCACCCTGTACTGCAACCACAGGAACGTGAGGAGCAAGAGGAGCAGGCTGCTGCACAGC |
| | GACTACATGAACATGACCCCCAGGCGACCCGGCCCTACCAGGAAACATTATCAGCCCTACGCCCCACCCAGGGACTTCGCCGCCTACAGGAGCAGGGTGA |
| | AGTTCAGCAGGAGCGCCGACGCCCCCGCCTACCAGCAGGGCCAGAACCAGCTGTACAATGAGCTGAACCTGGGCAGGAGGGAGGAGTACGACGTGCTGG |
| | ACAAGAGGAGGGGAAGGGACCCCGAGATGGGCGGAAAGCCCAGGAGGAAGAACCCCCAGGAGGGCCTGTACAACGAGCTCCAGAAGGACAAGATGGC |
| | CGAGGCCTATAGCGAGATCGGCATGAAGGGCGAGAGGAGGAGGGGCAAGGGCCACGACGGCCTGTACCAAGGCCTGAGCACCGCCACCAAGGACACCT |
| | ACGACGCTCTTCACATGCAGGCCCTGCCCCCTAGGTAATGATGA |
| 70 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTQCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGGGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGGGGGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTQGATCTGGTCCTTGC |
| | TATTGCACCQGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGQGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTCCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCTGCC |
| | CAGCTGCTGGGCCTGCTGCTGCTGTGGCTTCCTGGGGCCAAGTGCCAGGTGCAGCTGGTGCAGAGCGGCGCCGAGGTGAAGAAGCCTGGCGCCTCCGTG |
| | AAGGTGAGCTGCAAGGCCAGCGGCTACACCTTCACCGGCTACTACATCCACTGGGTGAGACAGGCCCCCGGCCAGGGACTGGAGTGGATGGGCTGGATC |
| | AACCCCAACAGCGGCGGCACCAACTACGCCCAGAAGTTCCAGGGCAGGGTGACAATGACCAGGGACACCAGCATCGGCACCGCCTACATGGAGCTGAGC |
| | AGGCTCAGGAGCGACGACACCGCCATCTACTACTGCACCAGGGAGGACACCTGGAACTACATCGACTACTGGGGCCAGGGAACCCTGGTGACAGTGAGC |
| | AGCGGAGGTGGCGGAAGCGGGGGGGGAGGATCAGGGGGAGGTGGAAGTGGCGGAGGAGGCAGCGACATCCAGATGACCCAGAGCCCCAGCTCCCTGA |
| | GCGCCAGCGTGGGCGACAGGGTGACCATCACCTGCAGGGCCTCCCAGAGCATCAGCAGCCACCTGAACTGGTACCAGCAGAAGCCCGGCAAGGCCCCCA |
| | AGCTGCTGATCTACGCCGCCAGCAGCCTCCACAGCGGCGTGCCCAGCAGGTTCTCTGGCTCTGGCAGCGGCACCGACTTCACCTTAACAATTTCTAGCCTGC |
| | AACCCGAGGACTTCGCTACCTACTGCTGCCAGCAGAGCTACAGCACCCCCAGAACCTTTGGCCAGGGCACCAAGGTGGAGATCAAGGCCGCCGCCATCGA |
| | GGTGATGTACCCTCCCCCTTACCTGGACAACGAGAAGAGCAACGGCACCATCATCCACGTGAAGGGCAAGCACCTGTGCCCCAGCCCCCTGTTCCCCGGCC |
| | CCTCTAAGCCCTTCTGGGTCCTGGTGGTGGTGGGGGGCGTGCTGGCCTGCTATAGCCTGCTGGTGACCGTGGCCTTCATCATCTTCTGGGTGAGGAGCAAG |
| | AGGAGCAGGCTGCTGCACAGCGATTACATGAACATGACCCCCAGGCGACCCGGCCCTACCAGGAAGCACTACCAGCCCTACGCCCCACCCAGGGACTTCG |
| | CCGCCTACAGGAGCAGGGTGAAGTTCAGCAGGAGCGCCGACGCCCCCGCTTACCAGCAGGGCCAGAACCAGCTGTACAATGAGCTGAACCTGGGCAGGA |
| | GGGAGGAGTACGACGTGCTGGACAAGAGGAGGGGAAGGGACCCCGAGATGGGGGGCAAGCCCAGGAGGAAGAACCCCCAGGAGGGCCTGTACAACGA |
| | GCTCCAGAAAGACAAGATGGCCGAGGCCTACAGCGAGATCGGCATGAAGGGCGAGAGGAGGAGGGGCAAGGGCCACGACGGCCTGTACCAGGGCCTG |
| | AGCACCGCCACCAAGGATACCTACGATGCCCTGCACATGCAGGCCCTGCCTCCTAGGTAATGATGA |
| 71 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTQGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATQCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGCCTGCTGTTGCTTTGGCTGCCCGGCGCTAAGTGCCAGGTGCAGCTGGTGCAGAGCGGCGCCGAGGTGAAGAAGCCTGGCGCCTCCGTG |
| | AAGGTGAGCTGCAAGGCCAGCGGCTACACCTTCACCGGCTACTACATCCACTGGGTGAGGCAGGCCCCCGGCCAGGGACTGGAGTGGATGGGCTGGATC |
| | AACCCCAACAGCGGCGGCACCAACTACGCCCAGAAGTTCCAGGGCAGGGTGACAATGACCAGGGACACCAGCATCGGCACCGCCTACATGGAGCTGAGC |
| | AGGCTCAGGAGCGACGACACCGCCATCTACTACTGCACCAGGGAGGACACCTGGAACTACATCGACTACTGGGGCCAGGGAACCCTGGTGACCGTGAGCA |
| | GCGGAGGGGGAGGAAGCGGCGGAGGAGGCAGTGGAGGAGGAGGATCCGGAGGCGGCGGAAGCGACATCCAGATGACCCAGAGCCCCAGCTCCCTGA |
| | GCGCCAGCGTGGGCGACAGGGTGACCATCACCTGCAGGGCCTCTCAGAGCATCAGCTCTCACCTGAATTGGTACCAGCAGAAGCCCGGCAAGGCCCCCAA |
| | GCTGCTGATCTACGCCGCCAGCAGCTTACACAGCGGCGTGCCCAGCAGGTTTTCTGGCAGCGGAAGCGGCACCGACTTCACCTTAACAATCTCCAGCCTGC |
| | AGCCCGAGGACTTTGCTACCTACTGCTGCCAGCAGAGCTACAGCACCCCCAGAACCTTCGGCCAGGGCACCAAGGTGGAAATCAAGGCCTCTTCTCTGAGA |
| | CCCGAGGCTTGCAGACCCGCCGCTGGAGGCGCCGTTCATACCAGGGGCCTGGACTTCGCTTGCGACATCTACATCTGGGCCCCCTTGGCCGGCACCTGCGG |
| | CGTGCTGCTGCTGAGCCTGGTGATCACCCTGTACTGCAACCACAGGAACGTGAGGAGCAAGAGGAGCAGGCTGCTGCATAGCGACTACATGAACATGACC |
| | CCCAGGCGACCCGGCCCCACCAGGAAGCACTACCAGCCCTACGCCCCACCCAGGGACTTCGCCGCCTACAGGAGCAGGGTGAAGTTCAGCAGGAGCGCTG |
| | ATGCCCCCGCCTACCAGCAGGGCCAGAACCAGCTGTACAATGAGCTGAATCTGGGCAGGAGGGAGGAGTACGACGTGCTGGACAAGAGGAGGGGAAGG |
| | GACCCCGAGATGGGGGGCAAGCCCAGGAGGAAGAACCCCCAGGAGGGCCTGTACAACGAGCTCCAGAAGGACAAGATGGCCGAGGCCTACAGCGAGAT |
| | CGGCATGAAGGGCGAGAGGAGGAGAGGCAAGGGCCACGACGGCCTGTACCAGGGCCTGAGCACCGCCACCAAAGACACCTACGACGCCCTGCACATGC |
| | AGGCCCTGCCCCCTAGGTAATGATGA |
| 72 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTQCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGACTGCTGCTCCTTTGGCTGCCCGGCGCTAAGTGCCAGGTGCAGCTGGTGCAGAGCGGCGCCGAGGTGAAGAAGCCTGGCGCCTCCGTGA |
| | AGGTGAGCTGCAAGGCCAGCGGCTACACCTTCACCGGCTACTACATCCACTGGGTGAGGCAGGCCCCCGGCCAGGGACTGGAGTGGATGGGCTGGATCA |
| | ACCCCAACAGCGGCGGCACCAACTACGCCCAGAAGTTCCAGGGCAGAGTGACCATGACCAGGGACACCAGCATCGGCACCGCCTACATGGAGCTGAGCA |
| | GGCTCAGGAGCGACGACACCGCCATCTACTACTGCACCAGGGAGGACACCTGGAACTACATCGACTACTGGGGCCAGGGCACCCTGGTGACCGTGAGCAG |
| | CGGAGGAGGCGGATCTGGCGGCGGAGGATCAGGAGGGGGAGGAAGCGGAGGCGGAGGCAGCGACATCCAGATGACCCAGAGCCCCAGCTCCCTGAGC |
| | GCCAGCGTGGGCGACAGGGTTACCATTACCTGCAGGGCCTCTCAGTCTATCAGCTCTCATCTGAACTGGTACCAGCAGAAGCCCGGCAAGGCCCCTAAGCT |
| | GCTGATCTATGCCGCCAGCAGCCTCCACAGCGGCGTGCCTAGCAGGTTCTCTGGCAGCGGAAGTGGCACCGACTTCACCTTAACCATCAGCTCACTGCAGC |
| | CCGAGGACTTCGCTACCTACTGCTGCCAGCAGAGCTACAGCACCCCCAGAACCTTCGGACAGGGCACCAAGGTGGAGATCAAGGQCTCCACAACAACACCC |
| | GCTCCCAGGCCCCCTACCCCCGCCCCCACAATTGCCAGCCAACCCCTCAGCCTGAGACCCGAAGCCTGTAGACCTGCCGCAGGGGGAGCCGTGCACACCAG |
| | GGGCCTGGACTTCGCATGCGACATCTACATCTGGGCCCCCTTAGCCGGCACCTGCGGCGTGCTGCTGCTGAGCCTTGTGATCACCCTGTACTGCAACCACAG |
| | GAACGTGAGGAGCAAGAGGAGCAGGCTGCTGCACAGCGACTACATGAACATGACCCCCAGGCGACCCGGCCCCACCAGGAAGCATTACCAGCCCTACGC |
| | CCCACCCAGGGACTTCGCCGCCTACAGGAGCAGGGTGAAGTTCAGCAGGAGCGCCGACGCCCCTGCCTACCAGCAGGGCCAGAACCAGCTGTACAATGAG |
| | CTGAACCTGGGCAGGAGGGAGGAGTATGACGTGCTGGACAAGAGGAGGGGAAGGGACCCCGAGATGGGGGGCAAGCCCAGGAGGAAGAACCCCCAGG |
| | AGGGCCTGTATAACGAGCTCCAGAAGGACAAGATGGCCGAAGCCTACAGCGAGATCGGCATGAAGGGCGAGAGGAGGAGGGGCAAGGGCCATGACGG |
| | CCTGTACCAGGGCCTGAGCACCGCCACCAAGGACACCTACGACGCCCTGCACATGCAGGCCCTGCCCCCTAGGTAATGATGA |
| 73 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGGGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGQGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTQTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGGGCCGCTGCCAAGCTTCCGAGCTCTCGAATTCATGGACATGAGGGTGCCCGCC |
| | CAGCTGCTGGGACTGCTGCTCCTTTGGCTGCCCGGCGCTAAGTGCCAGGTGCAGCTGGTGCAGAGCGGCGCCGAGGTGAAGAAGCCTGGCGCCTCCGTGA |
| | AGGTGAGCTGCAAGGCCAGCGGCTACACCTTCACCGGCTACTACATCCACTGGGTGAGGCAGGCCCCCGGCCAGGGACTGGAGTGGATGGGCTGGATCA |
| | ACCCCAACAGCGGCGGCACCAACTACGCCCAGAAGTTCCAGGGCAGAGTGACCATGACCAGGGACACCAGCATCGGCACCGCCTACATGGAGCTGAGCA |
| | GGCTCAGGAGCGACGACACCGCCATCTACTACTGCACCAGGGAGGACACCTGGAACTACATCGACTACTGGGGCCAGGGCACCCTGGTGACCGTGAGCAG |
| | CGGAGGAGGCGGATCTGGCGGCGGAGGATCAGGAGGGGGAGGAAGCGGAGGCGGAGGCAGCGACATCCAGATGACCCAGAGCCCCAGCTCCCTGAGC |
| | GCCAGCGTGGGCGACAGGGTTACCATTACCTGCAGGGCCTCTCAGTCTATCAGCTCTCATCTGAACTGGTACCAGCAGAAGCCCGGCAAGGCCCCTAAGCT |
| | GCTGATCTATGCCGCCAGCAGCCTCCACAGCGGCGTGCCTAGCAGGTTCTCTGGCAGCGGAAGTGGCACCGACTTCACCTTAACCATCAGCTCACTGCAGC |
| | CCGAGGACTTCGCTACCTACTGCTGCCAGCAGAGCTACAGCACCCCCAGAACCTTCGGACAGGGCACCAAGGTGGAGATCAAGGCCTCCACAACAACTAG |
| | CATCATGTACTTCAGCCATTTCGTGCCCGTGTTTCTTCCCGCTAAACCCACCACTACACCCGCTCCCAGGCCCCCTACCCCCGCCCCCACAATTGCCAGCCAAC |
| | CCCTCAGCCTGAGACCCGAAGCCTGTAGACCTGCCGCAGGGGGAGCCGTGCACACCAGGGGCCTGGACTTCGCATGCGACATCTACATCTGGGCCCCCTTA |
| | GCCGGCACCTGCGGCGTGCTGCTGCTGAGCCTTGTGATCACCCTGTACTGCAACCACAGGAACGTGAGGAGCAAGAGGAGCAGGCTGCTGCACAGCGACT |
| | ACATGAACATGACCCCCAGGCGACCCGGCCCCACCAGGAAGCATTACCAGCCCTACGCCCCACCCAGGGACTTCGCCGCCTACAGGAGCAGGGTGAAGTT |
| | CAGCAGGAGCGCCGACGCCCCTGCCTACCAGCAGGGCCAGAACCAGCTGTACAATGAGCTGAACCTGGGCAGGAGGGAGGAGTATGACGTGCTGGACAA |
| | GAGGAGGGGAAGGGACCCCGAGATGGGGGCAAGCCCAGGAGGAAGAACCCCCAGGAGGGCCTGTATAACGAGCTCCAGAAGGACAAGATGGCCGAA |
| | GCCTACAGCGAGATCGGCATGAAGGGCGAGAGGAGGAGGGGCAAGGGCCATGACGGCCTGTACCAGGGCCTGAGCACCGCCACCAAGGACACCTACGA |
| | CGCCCTGCACATGCAGGCCCTGCCCCCTAGGTAATGATGA |
| 74 | TTCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCQGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATQCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCTGCC |
| | CAGTTGTTGGGCCTGCTGCTGCTGTGGCTGCCCGGCGCCAAGTGCCAGGTTACCCTGAGGGAGTCTGGCCCCGCCCTGGTGAAGCCCACCCAGACCTTAAC |
| | CCTGACCTGCACCTTCAGCGGCATCAGCCTGACCACCAGCGGCATGTGCGTGAGCTGGATCAGGCAGCCCCCAGGCAAGGCCCTGGAGTGGCTGGCCCTG |
| | ATCGACTGGGACGGCGACAACTACTACAGCACCAGCCTGAAGACCAGGCTGACCATCTCCAAGGACACCAGCAAGAACCAGGTGGTGCTGACCATGGCCA |
| | GCATGGACCCCGTGGACACCGCCACATACTACTGCGCCAGGATGGGCTACACCTACGGCTACGACGCTTTCGACATCTGGGGCCAGGGCACCATGGTGAC |
| | AGTGAGCAGCGGCGGTGGAGGATCTGGCGGAGGCGGAAGCGGAGGAGGCGGATCTGGGGGGGGAGGAAGCGAGATTGTGCTGACCCAGACCCCCAGC |
| | AGCCTCAGCGCCAGCCTGGGCGACAGGGTGACCATCAGCTGCAGGGCCTCTCAGAGCATCGGCAGCCTGTTACACTGGTACCAGCAGAAGCCCGACCAGA |
| | GCCCCAAGCTGCTGATCAAGTTCGCCAGCCAGAGCCTGAGCGGCGTGCCCAGCAGGTTCTCTGGCTCTGGCAGCGGCACCGACTTCACCTTAACCATCAAC |
| | AGCCTGGAGGCCGAAGACGCCGCCACCTACTACTGCCACCAGAGCAGGAGCCTGCCCTTTACCTTCGGCCCCGGCACCAAGGTTGACATCAACGCCGCCGC |
| | CATCGAGGTGATGTACCCTCCCCCTTACCTGGACAACGAGAAGAGCAACGGCACCATCATCCACGTGAAGGGCAAGCACCTGTGCCCCAGCCCCCTGTTCC |
| | CCGGCCCCTCTAAGCCCTTCTGGGTTCTGGTGGTGGTGGGCGGCGTGCTGGCCTGCTACAGCCTGCTGGTGACCGTGGCCTTCATCATCTTCTGGGTGAGG |
| | AGTAAGAGGAGCAGGCTGCTGCACAGCGACTACATGAACATGACCCCCAGGCGACCCGGCCCCACCAGGAAGCACTACCAGCCCTACGCCCCTCCAAGGG |
| | ACTTCGCCGCCTACAGGAGCAGGGTGAAGTTCAGCAGGAGCGCCGACGCCCCCGCCTACCAGCAGGGCCAGAACCAGCTGTACAATGAGCTGAACCTGG |
| | GCAGGAGGGAGGAGTACGACGTGCTGGACAAGAGGAGGGGAAGGGACCCCGAGATGGGCGGCAAGCCCAGGAGGAAGAACCCCCAGGAGGGCCTGT |
| | ACAACGAGCTCCAGAAGGACAAAATGGCCGAAGCCTACAGCGAGATCGGCATGAAGGGCGAGAGGAGGAGGGGCAAAGGCCACGACGGCCTGTACCAG |
| | GGCCTGAGCACCGCCACCAAAGACACCTACGACGCCCTGCACATGCAGGCCCTGCCCCCTAGGTAATGATGA |
| 75 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTQCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTQGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTCCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTQCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGCCTGCTGTTACTTTGGCTGCCCGGCGCCAAGTGCCAAGTGACCCTGAGGGAGTCTGGCCCCGCCCTGGTGAAGCCCACCCAGACCCTCAC |
| | CCTGACCTGCACCTTCAGCGGCATCAGCCTGACCACCAGCGGCATGTGCGTGAGCTGGATCAGGCAGCCCCCTGGCAAGGCCCTGGAGTGGCTGGCCCTG |
| | ATCGACTGGGACGGCGACAACTACTACAGCACCAGCCTGAAGACCAGGCTGACCATCTCCAAGGACACCAGCAAGAACCAGGTGGTGCTGACCATGGCCA |
| | GCATGGACCCCGTGGACACCGCCACATACTACTGCGCCAGGATGGGCTACACCTACGGCTACGACGCTTTCGACATCTGGGGCCAGGGCACCATGGTGAC |
| | CGTGAGCAGCGGCGGAGGAGGATCTGGAGGCGGAGGCTCTGGTGGAGGAGGCAGCGGGGGGGGAGGAAGCGAGATTGTGCTGACACAGACCCCCAGC |
| | TCCCTGAGCGCCAGCCTGGGCGACAGGGTGACCATCAGCTGCAGGGCCTCCCAGAGCATCGGCAGCCTGTTGCACTGGTACCAGCAGAAGCCCGATCAGA |
| | GCCCCAAACTGCTGATCAAGTTCGCCAGCCAGAGCCTGAGCGGCGTGCCCAGCAGGTTCTCTGGAAGCGGCTCTGGCACCGACTTCACCTTAACCATCAAC |
| | AGCCTGGAGGCCGAGGACGCCGCCACCTACTACTGCCACCAAAGCAGGAGCCTGCCCTTTACCTTCGGCCCCGGCACCAAGGTGGACATCAACGCCAGCA |
| | GCCTCAGGCCCGAAGCTTGTAGGCCTGCTGCCGGGGCGCTGTTCACACCAGGGGCTTAGACTTTGCTTGCGACATCTACATCTGGGCCCCCTTAGCCGGA |
| | ACCTGCGGCGTGCTGCTGCTGAGCCTGGTGATCACCCTGTACTGCAACCACAGGAATGTGAGGAGCAAGAGGAGCAGGCTGCTGCACAGCGACTACATGA |
| | ACATGACCCCCAGGCGACCCGGCCCCACCAGGAAGCACTACCAGCCCTACGCCCCACCCAGGGACTTCGCCGCCTACAGGAGCAGGGTGAAGTTCAGCAG |
| | GAGCGCCGACGCCCCCGCCTACCAGCAGGGCCAGAATCAGCTGTACAATGAGCTGAACCTGGGCAGGAGGGAGGAGTACGACGTGCTGGACAAGAGGA |
| | GGGGAAGGGACCCTGAGATGGGCGGCAAGCCCAGGAGGAAGAACCCCCAGGAGGGCCTGTACAACGAGCTCCAGAAGGACAAGATGGCCGAGGCCTAC |
| | AGCGAGATCGGCATGAAGGGCGAGAGGAGGAGGGGCAAGGGCCACGACGGCCTGTACCAGGGCTTAAGCACCGCCACCAAGGATACCTACGACGCCCT |
| | GCACATGCAGGCCCTGCCCCCTAGGTAATGATGA |
| 76 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGGGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTQCCACAGCTC |
| | CTGGGCAACGTGCTGGTCTGTGTGCTGGCCCATCACTTTGGCAAAGGGGGCCGCTGCCAAGCTTCCGAGCTCTCGAATTCATGGACATGAGGGTGCCCGCC |
| | CAGCTTCTGGGCCTGCTGCTCTTGTGGCTGCCTGGCGCCAAGTGCCAGGTGACCCTGAGGGAGTCTGGCCCCGCCCTGGTGAAGCCCACCCAGACCTTGAC |
| | CCTGACCTGCACCTTCAGCGGCATCAGCCTGACCACCAGCGGCATGTGCGTGAGCTGGATCAGGCAGCCCCCTGGCAAGGCCCTGGAGTGGCTGGCCCTG |
| | ATCGACTGGGACGGCGACAACTACTACAGCACCAGCCTGAAGACCAGGCTGACCATCTCTAAGGACACCAGCAAGAACCAGGTGGTGCTGACCATGGCCA |
| | GCATGGACCCCGTGGACACCGCCACATACTACTGCGCCAGGATGGGCTACACCTACGGCTACGACGCTTTCGACATCTGGGGCCAGGGCACCATGGTGAC |
| | CGTGAGCTCTGGGGGGGGAGGAAGCGGCGGCGGAGGATCTGGTGGAGGTGGGTCTGGAGGCGGGGGCAGCGAAATCGTGCTGACCCAGACCCCCAGC |
| | AGCCTCAGCGCCAGCCTGGGCGACAGGGTGACCATCAGCTGCAGGGCCTCCCAGAGCATCGGCAGCCTGTTGCACTGGTACCAGCAGAAGCCCGACCAGA |
| | GCCCCAAGCTGCTGATCAAGTTCGCCAGCCAGAGCCTGAGCGGCGTGCCCAGCAGGTTCTCTGGAAGCGGCTCTGGAACCGACTTCACCTTAACCATCAAC |
| | AGCCTGGAGGCCGAAGACGCCGCCACCTATTACTGCCACCAGAGCAGGAGCCTGCCCTTCACCTTCGGACCCGGAACCAAGGTGGACATCAACGCCAGCA |
| | CAACCACACCCGCCCCTAGGCCCCCTACCCCCGCCCCCACAATCGCTAGCCAACCCCTGAGCCTTAGGCCCGAAGCCTGTAGACCTGCCGCCGGAGGCGCC |
| | GTGCATACCAGGGGCCTGGACTTCGCTTGTGACATCTACATCTGGGCCCCCTTAGCCGGAACCTGCGGCGTGCTGCTGCTGAGCCTGGTGATCACCCTGTA |
| | CTGCAACCACAGGAACGTGAGGAGCAAAAGGAGCAGGCTGCTGCACAGCGACTACATGAACATGACCCCCAGGAGGCCCGGCCCCACCAGGAAGCACTA |
| | CCAGCCCTACGCCCCACCCAGGGACTTCGCCGCCTACAGGAGCAGGGTGAAGTTTAGCAGGAGCGCCGACGCCCCCGCCTACCAGCAGGGCCAGAATCAG |
| | CTGTACAATGAATTAAACCTGGGCAGGAGGGAGGAGTACGACGTGCTGGATAAGAGGAGGGGAAGGGACCCCGAGATGGGGGGCAAGCCCAGGAGGA |
| | AGAACCCTCAGGAGGGACTGTACAACGAGCTCCAGAAGGATAAGATGGCCGAGGCCTACAGCGAGATCGGCATGAAGGGCGAGAGGAGGAGGGGCAA |
| | GGGCCACGACGGCCTGTACCAAGGCCTGAGCACCGCCACCAAGGACACCTACGACGCCCTGCACATGCAGGCCCTGCCCCCTAGATAATGATGA |
| 77 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGGGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGGGGCCGCTGCCAAGCTTCCGAGCTCTCGAATTCATGGACATGAGGGTGCCCGCC |
| | CAACTGCTGGGCCTGCTGCTCCTTTGGCTGCCTGGCGCCAAGTGCCAGGTGACCCTGAGGGAGTCTGGCCCCGCCCTGGTGAAGCCCACCCAGACCTTGAC |
| | CCTGACCTGCACCTTCAGCGGCATCAGCCTGACCACCAGCGGCATGTGCGTGAGCTGGATCAGGCAGCCCCCTGGCAAGGCCCTGGAGTGGCTGGCCCTG |
| | ATCGACTGGGACGGCGACAACTACTACAGCACCAGCCTGAAGACCAGGCTGACCATCTCTAAGGACACCAGCAAGAACCAGGTGGTGCTGACCATGGCCA |
| | GCATGGACCCCGTGGACACCGCCACATACTACTGCGCCAGGATGGGCTACACCTACGGCTACGACGCTTTCGACATCTGGGGCCAGGGCACCATGGTGAC |
| | CGTGAGCAGCGGCGGAGGCGGTTCTGGTGGCGGAGGCTCAGGAGGCGGAGGATCTGGAGGCGGCGGCAGCGAAATCGTGCTGACCCAGACCCCCAGCA |
| | GCCTCAGCGCCAGCCTCGGCGACAGGGTGACCATCAGCTGCAGGGCCTCCCAGAGCATCGGCAGCCTGCTGCACTGGTACCAGCAGAAGCCCGACCAGAG |
| | CCCCAAGCTGCTGATCAAGTTCGCCAGCCAGAGCCTGAGCGGCGTGCCCAGCAGGTTCTCTGGCAGCGGCTCTGGCACCGACTTCACCTTAACCATCAACA |
| | GCCTGGAGGCCGAGGACGCCGCCACCTACTACTGCCATCAAAGCAGGAGCCTGCCCTTCACCTTCGGCCCCGGCACAAAAGTGGACATCAACGCCAGCCTG |
| | AGCAACAGCATCATGTACTTTAGCCATTTTGTGCCCGTGTTCCTGCCTGCCAAGCCCACAACAACCCCCGCCCCCAGGCCTCCTACCCCTGCCCCTACAATCG |
| | CTAGCCAACCCCTGTCTCTGAGACCCGAGGCCTGCAGACCTGCTGCCGGAGGCGCCGTGCACACCAGGGGACTGGACTTCGCTTGCGACATCTATATCTGG |
| | GCCCCCTTAGCCGGCACCTGCGGCGTGCTGCTGCTGAGCCTGGTGATTACCCTGTACTGCAACCACAGGAACGTGAGGAGCAAGAGGAGCAGACTGCTGC |
| | ACAGCGACTACATGAACATGACCCCCAGGCGACCCGGCCCCACCAGGAAGCACTACCAGCCCTACGCCCCACCCAGGGACTTCGCCGCCTACAGGAGCAG |
| | GGTGAAGTTCAGCAGGTCAGCCGACGCCCCCGCCTACCAACAGGGCCAGAACCAGCTGTACAATGAGCTGAACCTGGGCAGGAGGGAGGAGTACGACGT |
| | GCTGGACAAGAGGAGGGGAAGGGATCCTGAGATGGGCGGCAAGCCCAGGAGGAAGAACCCCCAGGAGGGCCTGTACAACGAGCTCCAGAAGGACAAG |
| | ATGGCCGAGGCCTACAGCGAGATCGGCATGAAGGGCGAGAGGAGGAGGGGCAAGGGCCACGACGGCCTGTACCAGGGCCTGAGCACCGCTACCAAGGA |
| 78 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCATATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTQGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGCCTGTTACTGCTGTGGCTGCCCGGCGCTAAGTGCCAGGTGACCCTGAGGGAGTCTGGCCCCGCCCTGGTGAAGACCACCCAGACCCTGA |
| | CACTGACCTGCACCTTCAGCGGCTTCAGCCTGTCCACCAGCGGCATGTGCGTGAGCTGGATCAGGCAGCCCCCAGGCAAGGCCTTGGAGTGGCTGGCCCT |
| | GATCGACTGGGACGACGACAAGTACTACAGCACCAGCCTGAAGACCAGGCTGACCATCTCTAAGGACACCAGCAAGAGCCAGGTGGTGCTGACCATGACC |
| | AGCATGGACCCCGTGGACACCGCCACATACTACTGCGCCAGGATCAGGTACATCTACGGCTACGACTACTTCGACTACTGGGGCCAGGGAACCCTGGTCAC |
| | CGTGAGCAGCGGAGGCGGTGGATCTGGAGGCGGAGGAAGCGGCGGAGGAGGATCCGGAGGGGGGGGCAGCGATATCCAGATGACCCAGAGCCCCAGC |
| | TCCCTGAGCGCCTCTGTGGGCGACAGGGTGACCATCACCTGCAGGGCCTCTCAGAGGATCAGCTCTCAGCTGAACTGGTACCAGCAGAAGCCCGGCAAGG |
| | CCCCCAAGCTGCTGATCTACGCCGCCAGCAGCCTCCAGAGCGGCGTGCCCTCTAGGTTCTCTGGCTCTGGAAGCGGCACCGAGTTCACCCTGACCATTAGC |
| | AGCTTGCAGCCCGAGGACATCGCCACCTACTGCTGCCAGCAGACCTACAGCAAGCCTATCACCTTCGGCCAGGGCACCAGGCTGGAGATCAAGGCCGCCG |
| | CCATCGAGGTGATGTACCCTCCCCCTTACCTGGACAACGAGAAGAGCAACGGCACCATCATCCACGTGAAGGGCAAGCACCTGTGCCCCAGCCCCCTGTTC |
| | CCCGGCCCCTCTAAGCCCTTCTGGGTCCTGGTGGTGGTGGGCGGCGTGCTGGCCTGCTACAGCCTGCTGGTGACCGTGGCCTTCATCATCTTCTGGGTGAG |
| | GAGCAAGAGGAGCAGGCTGCTGCACAGCGACTACATGAACATGACCCCCAGGCGACCCGGCCCCACCAGGAAGCACTACCAGCCCTACGCCCCACCCAGG |
| | GACTTCGCCGCCTACAGGAGCAGGGTGAAGTTCAGCAGGAGCGCCGACGCCCCCGCCTACCAGCAGGGCCAGAACCAGCTGTACAATGAGCTGAACCTG |
| | GGCAGGAGGGAGGAGTACGACGTGCTGGACAAGAGGAGGGGAAGGGACCCCGAGATGGGCGGCAAGCCCAGGAGGAAGAACCCCCAGGAGGGCCTG |
| | TACAACGAGCTCCAGAAGGACAAGATGGCCGAGGCCTACAGCGAGATCGGCATGAAGGGCGAGAGGAGGAGGGGCAAGGGCCACGACGGCCTGTACCA |
| | GGGCCTGAGCACCGCCACCAAGGACACCTACGACGCCCTGCACATGCAGGCCTTACCCCCTAGGTAATGATGA |
| 79 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGGGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTQCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTQGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGQGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAACTGCTGGGCCTGTTGCTGCTTTGGCTGCCCGGCGCCAAGTGCCAGGTGACCCTGAGGGAGTCTGGACCCGCCCTGGTGAAGACCACCCAGACCCTGA |
| | CACTGACCTGCACCTTCAGCGGCTTCAGCCTGTCCACCAGCGGCATGTGCGTGAGCTGGATCAGGCAGCCCCCTGGCAAGGCCTTGGAGTGGCTGGCCCTG |
| | ATCGACTGGGACGACGACAAGTACTACAGCACCAGCCTGAAGACCAGGCTGACCATCAGCAAGGACACCAGCAAGAGCCAGGTGGTGCTGACCATGACC |
| | AGCATGGACCCCGTGGACACCGCCACATACTACTGCGCCAGGATCAGGTACATCTACGGCTACGACTACTTCGACTACTGGGGCCAGGGCACCCTGGTGAC |
| | CGTGAGCAGCGGAGGCGGAGGATCTGGCGGAGGGGGATCTGGAGGTGGAGGCTCTGGCGGCGGCGGCAGCGATATCCAGATGACCCAGAGCCCCAGCT |
| | CCCTGAGCGCCAGCGTGGGCGACAGGGTGACCATCACCTGCAGGGCCTCCCAGAGGATCAGCTCCCAGCTGAACTGGTACCAGCAGAAGCCCGGCAAGG |
| | CCCCCAAGCTGCTGATCTACGCCGCCAGCAGCCTTCAGAGCGGCGTGCCCAGCAGGTTCTCCGGCAGCGGGAGCGGCACCGAGTTCACCCTGACCATTAGC |
| | TCACTGCAGCCCGAGGACATCGCCACCTACTGCTGCCAGCAGACCTACAGCAAGCCCATCACCTTCGGGCAGGGCACCAGGCTGGAGATCAAGGCCTCTTC |
| | TTTGAGACCCGAGGCTTGTAGGCCCGCAGCCGGCGGCGCCGTGCACACAAGGGGACTGGATTTCGCTTGCGACATCTATATCTGGGCCCCCTTAGCCGGCA |
| | CCTGCGGCGTGCTGCTGCTGAGCCTGGTGATCACCCTGTACTGCAACCACAGGAACGTGAGGAGCAAGAGGAGCAGGCTGCTGCACAGCGACTACATGAA |
| | CATGACACCCAGGAGGCCCGGCCCTACCAGGAAGCACTACCAGCCCTACGCCCCACCCAGGGACTTQGCCGCTTATAGGAGCAGGGTGAAGTTCAGCAGG |
| | AGCGCCGACGCTCCCGCCTACCAGCAGGGCCAGAACCAGCTGTACAATGAGCTGAACCTGGGCAGGAGGGAGGAGTACGACGTGCTGGACAAGAGAAG |
| | GGGAAGGGACCCCGAGATGGGGGGCAAGCCCAGGAGGAAGAACCCCCAGGAGGGCCTGTACAACGAGCTCCAGAAGGACAAGATGGCCGAGGCCTACA |
| | GCGAGATCGGCATGAAGGGCGAGAGGAGGAGGGGCAAGGGCCACGACGGCCTGTACCAGGGCCTGAGCACCGCCACCAAAGACACCTACGACGCCCTG |
| | CACATGCAGGCCCTGCCCCCTAGGTAATGATGA |
| 80 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGGGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGGGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTQCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGTTGGGCCTGCTGCTGCTTTGGCTGCCTGGCGCCAAGTGCCAGGTGACCCTGAGGGAGTCTGGCCCCGCCCTGGTGAAGACCACCCAGACCCTGAC |
| | ACTGACCTGCACCTTCAGCGGCTTCAGCCTGTCCACCAGCGGCATGTGCGTGAGCTGGATCAGGCAGCCCCCTGGAAAGGCCCTGGAGTGGCTGGCCCTG |
| | ATCGACTGGGACGACGACAAGTACTACAGCACCAGCCTGAAGACCAGGCTGACCATCTCTAAGGACACCAGCAAGAGCCAGGTGGTGCTGACCATGACCA |
| | GCATGGACCCCGTGGACACCGCCACATACTACTGCGCCAGGATCAGGTACATCTACGGCTACGACTACTTCGACTACTGGGGCCAGGGCACCCTGGTGACC |
| | GTGAGCTCTGGCGGGGGAGGAAGCGGAGGTGGGGGAAGCGGTGGAGGAGGATCTGGGGGGGGCGGCAGCGATATCCAGATGACCCAGAGCCCCAGCT |
| | CCCTGAGCGCCAGCGTGGGCGACAGGGTGACCATCACCTGCAGGGCCTCCCAGAGGATCAGCAGCCAGCTGAACTGGTACCAGCAGAAGCCCGGCAAGG |
| | CCCCTAAGCTCCTGATCTACGCCGCCAGCAGCCTCCAGAGCGGCGTGCCCAGCAGGTTCTCCGGCTCTGGAAGCGGAACCGAGTTCACCTTGACCATCAGT |
| | AGCCTGCAGCCCGAGGATATCGCCACCTACTGCTGCCAGCAGACCTACAGCAAACCCATCACCTTCGGCCAAGGCACCAGGCTGGAGATCAAGGCCAGCA |
| | CCACAACCCCTGCCCCTAGGCCCCCTACACCCGCTCCCACCATCGCTAGCCAACCTCTGTCTCTGAGACCTGAGGCTTGCAGACCTGCTGCCGGCGGCGCCG |
| | TGCACACCAGAGGCCTGGACTTCGCTTGCGACATCTACATTTGGGCCCCCTTAGCCGGAACCTGCGGCGTTCTGCTGCTGAGCCTGGTGATCACCCTGTACT |
| | GCAACCACAGGAACGTGAGGAGCAAGAGGAGCAGGCTGCTGCATAGCGACTACATGAACATGACCCCCAGGCGACCCGGCCCCACCAGGAAGCACTACC |
| | AGCCCTATGCCCCACCCAGGGACTTCGCCGCTTACAGGAGCAGGGTGAAGTTCAGCAGGAGCGCCGACGCCCCCGCCTACCAGCAGGGCCAGAACCAGCT |
| | GTACAATGAGCTGAACCTGGGCAGGAGGGAGGAGTACGACGTGCTGGACAAGAGGAGGGGAAGGGACCCCGAGATGGGGGCAAGCCCAGGAGGAAG |
| | AACCCCCAGGAGGGCCTGTACAACGAGCTCCAGAAGGACAAGATGGCCGAGGCCTACAGCGAGATCGGCATGAAGGGCGAGAGGAGGAGGGGCAAGG |
| | GCCACGACGGCCTGTACCAGGGCCTGAGCACCGCCACCAAGGACACCTACGACGCCCTGCACATGCAGGCCCTGCCCCCTAGGTAATGATGA |
| 81 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTQCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTCCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGCCTGCTTCTGTTATGGCTGCCCGGCGCCAAGTGCCAGGTGACCCTGAGGGAGTCTGGACCCGCCCTGGTGAAGACCACCCAGACCCTGA |
| | CACTGACCTGCACCTTCAGCGGCTTCAGCCTGTCCACCAGCGGCATGTGCGTGAGCTGGATCAGGCAGCCCCCTGGAAAGGCCCTGGAGTGGCTGGCCCT |
| | GATCGACTGGGACGACGACAAGTACTACAGCACCAGCCTGAAGACCAGGCTGACCATCTCTAAGGACACCAGCAAGAGCCAGGTGGTGCTGACCATGACC |
| | AGCATGGACCCCGTGGACACCGCCACATACTACTGCGCCAGGATCAGGTACATCTACGGCTACGACTACTTCGACTACTGGGGCCAGGGCACCCTGGTGAC |
| | CGTGAGCAGCGGTGGAGGAGGCTCTGGCGGAGGAGGGTCCGGAGGAGGTGGATCTGGAGGCGGCGGCAGCGACATCCAGATGACCCAGAGCCCCAGC |
| | TCCCTGAGCGCCAGCGTGGGCGACAGGGTGACCATCACCTGCAGGGCCTCCCAGAGGATCAGCTCCCAGCTGAACTGGTACCAGCAGAAGCCCGGCAAG |
| | GCCCCCAAGCTGCTGATCTACGCCGCCAGCAGCCTCCAGAGCGGCGTGCCCAGCAGGTTCTCCGGAAGCGGCAGCGGCACCGAGTTCACCCTGACCATTTC |
| | CAGCCTGCAGCCCGAGGACATCGCCACCTACTGCTGCCAGCAGACCTACAGCAAGCCCATCACCTTCGGCCAAGGCACCAGGCTGGAGATCAAGGCCAGC |
| | CTGAGCAACAGCATCATGTACTTCAGCCACTTCGTGCCCGTGTTCCTGCCCGCCAAGCCCACAACCACCCCTGCTCCCAGACCCCCTACACCCGCCCCCACCA |
| | TCGCCAGCCAACCCCTTTCTTTGAGACCCGAGGCCTGCAGACCCGCCGCCGGCGGAGCTGTGCATACAAGGGGCTTGGACTTCGCTTGCGACATCTACATC |
| | TGGGCCCCCTTAGCCGGCACCTGCGGCGTGCTGCTGCTGAGCCTGGTGATCACCCTGTACTGCAACCACAGGAACGTGAGGAGCAAGAGGAGCAGGCTGC |
| | TGCACAGCGACTACATGAACATGACCCCCAGGAGGCCCGGCCCCACCAGGAAGCACTACCAGCCCTATGCTCCACCCAGGGATTTTGCCGCCTACAGGAGC |
| | CGTGCTGGACAAGAGGAGGGGAAGGGACCCCGAGATGGGCGGCAAGCCCAGGAGGAAGAACCCTCAGGAGGGCCTGTACAACGAGCTCCAGAAGGAC |
| | AGGGTGAAGTTCAGCAGGAGCGCCGACGCCCCCGCCTACCAGCAGGGCCAGAACCAGCTGTACAATGÅGCTGAACCTGGGCAGGAGGGAGGAGTATGÅ |
| | CGTGCTGGACAAGAGGAGGGGAAGGGACCCCGAGATGGGCGGCAAGCCCAGGAGGAAGAACCCTCAGGAGGGCCTGTACAACGAGCTCCAGAAGGAC |
| | AAGATGGCCGAGGCCTACAGCGAGATCGGCATGAAGGGCGAGAGGAGGAGGGGCAAGGGCCACGACGGCTTGTACCAGGGCCTGAGCACCGCCACCAA |
| | GGACACCTACGACGCCCTGCACATGCAGGCCCTGCCCCCTAGGTAATGATAA |
| 82 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGGGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTGGGACTTCTGCTGCTGTGGCTTCCCGGCGCCAAGTGCCAGGTGCAGCTGCAGCAGAGCGGCCCCGGACTGGTGAAGCCCAGCCAGACCCTGA |
| | GCCTGACCTGCGCCATCAGCGGCGACAGCGTGAGCAGCAACAGCGCCGCCTGGAACTGGATCAGGCAGAGCCCCAGCAGGGGCCTGGAGTGGCTGGGC |
| | AGAACCTACTACAGGTCCAAGTGGTACAACGACTACGCCGTGAGCGTGAAGAGCAGGATCACCATCAACCCCGACACCAGCAAGAACCAGTTCTCACTGC |
| | AGCTGAACAGCGTGACCCCCGAGGACACCGCCGTGTACTACTGCGCCAGGGAGGTGACCGGCGACCTGGAGGACGCCTTCGACATCTGGGGCCAGGGAA |
| | CCATGGTGACAGTGAGCAGCGGCGGAGGCGGCTCAGGAGGCGGCGGATCTGGTGGAGGAGGAAGTGGTGGAGGTGGCAGCGACATCCAGATGACACA |
| | GAGCCCTTCTAGCCTGAGCGCCAGCGTGGGCGACAGGGTGACCATCACATGCAGGGCCTCTCAGACCATCTGGAGCTATCTGAACTGGTACCAGCAGAGG |
| | CCCGGAAAGGCCCCCAACCTGCTGATCTACGCCGCCAGCAGCCTCCAGAGCGGCGTGCCCAGCAGGTTCTCTGGCAGGGGCAGCGGCACCGACTTCACCT |
| | TAACCATCAGCAGCCTTCAGGCCGAGGACTTCGCTACCTACTACTGCCAGCAGAGCTACAGCATCCCCCAGACCTTCGGCCAGGGCACCAAGCTGGAGATC |
| | AAGGCCGCCGCCATCGAGGTGATGTACCCTCCCCCTTACCTGGACAACGAGAAGTCTAACGGCACCATCATCCACGTGAAGGGCAAGCACCTGTGCCCCAG |
| | CCCCCTGTTCCCCGGCCCCTCTAAGCCCTTCTGGGTCCTGGTGGTGGTGGGGGGCGTGCTGGCCTGCTACAGCCTGCTGGTGACCGTGGCCTTCATCATCTT |
| | CTGGGTGAGGAGCAAGAGGAGCAGGCTGCTGCACAGCGACTACATGAATATGACCCCCAGGCGACCCGGCCCCACCAGGAAACACTACCAGCCCTACGCC |
| | CCACCCAGGGACTTCGCCGCCTACAGGAGCAGGGTGAAATTCAGCAGGAGCGCCGACGCCCCCGCCTACCAGCAGGGCCAGAACCAGCTGTACAATGAGC |
| | TGAACCTGGGCAGGAGGGAGGAGTACGACGTGCTGGACAAGAGGAGGGGAAGGGACCCCGAGATGGGGGGCAAGCCCAGGAGGAAGAACCCCCAGGA |
| | GGGCCTGTACAACGAGCTCCAGAAGGACAAGATGGCCGAGGCCTACAGCGAGATCGGCATGAAGGGCGAGAGGAGGAGGGGCAAGGGCCACGACGGC |
| | CTGTACCAGGGCCTGAGCACCGCCACCAAGGACACCTACGACGCCCTGCACATGCAAGCTCTGCCCCCTAGGTAATGATGA |
| 83 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAAGCCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGGGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGGGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGQGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGCGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGGGCCGCTGCCAAGCTTCCGAGCTCTCGAATTCATGGACATGAGGGTTCCCGCC |
| | CAGCTGCTGGGCCTGCTGCTCCTTTGGCTGCCCGGAGCCAAGTGCCAGGTGCAGCTGCAGCAGAGCGGACCCGGCCTGGTGAAGCCCAGCCAGACCCTGA |
| | GCCTGACCTGCGCCATCAGCGGCGACAGCGTGAGCAGCAACAGCGCCGCCTGGAACTGGATCAGGCAGAGCCCCAGCAGGGGCCTGGAGTGGCTGGGC |
| | AGAACCTACTACAGGTCCAAGTGGTACAACGACTACGCCGTGAGCGTGAAGAGCAGGATCACCATCAACCCCGACACCAGCAAGAACCAGTTCTCACTGC |
| | AGCTGAACAGCGTGACCCCCGAGGACACCGCCGTGTACTACTGCGCCAGGGAGGTGACCGGCGACCTGGAGGACGCCTTCGACATCTGGGGCCAGGGAA |
| | CCATGGTGACCGTGAGCTCCGGAGGCGGAGGATCTGGAGGTGGCGGAAGCGGCGGAGGTGGCTCTGGCGGAGGGGGCAGCGATATCCAGATGACCCAG |
| | AGCCCCTCCAGCCTGAGCGCCAGCGTGGGCGACAGGGTGACCATCACCTGCAGGGCCTCCCAGACCATCTGGAGCTACCTGAACTGGTACCAGCAGAGGC |
| | CCGGCAAGGCCCCCAACCTGCTGATCTACGCCGCCAGCAGCTTACAAAGCGGCGTGCCCAGCAGGTTCTCTGGCAGGGGCAGCGGCACCGACTTCACCTTA |
| | ACCATCAGCAGCCTGCAGGCCGAGGACTTCGCTACCTACTACTGCCAGCAGAGCTACAGCATCCCCCAGACCTTCGGCCAGGGCACCAAGCTGGAAATCAA |
| | AGCTAGCAGCCTTAGACCCGAGGCCTGCAGACCCGCCGCCGGCGGAGCTGTGCATACAAGGGGCCTTGATTTTGCTTGCGACATCTACATCTGGGCCCCCT |
| | TAGCCGGCACCTGCGGCGTGCTGCTGCTGAGCCTGGTGATCACCCTGTACTGCAACCACAGGAACGTGAGGAGCAAGAGGAGCAGGCTTCTGCACAGCGA |
| | CTACATGAATATGACCCCCAGGCGACCCGGCCCCACCAGGAAGCACTATCAGCCCTACGCCCCACCCAGGGACTTCGCCGCCTACAGGAGCAGGGTGAAG |
| | TTCAGCAGGAGCGCCGACGCCCCCGCCTACCAGCAGGGCCAGAACCAGCTGTACAATGAGCTGAACCTTGGCAGGAGGGAGGAGTACGACGTGCTTGAT |
| | AAGAGGAGGGGAAGGGACCCCGAGATGGGGGGCAAGCCCAGGAGGAAGAACCCCCAGGAGGGCCTGTACAACGAGCTCCAGAAGGACAAGATGGCCG |
| | AGGCCTACTCTGAGATCGGCATGAAGGGCGAGAGGAGGAGGGGCAAGGGCCACGATGGCCTGTACCAGGGCCTGAGCACCGCCACCAAGGACACCTAC |
| | GACGCCCTGCACATGCAGGCCCTGCCCCCTAGGTAATGATGA |
| 84 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTCGGATGGAAGCCGGTCTTGTCGATCAGGATGATCTGGACGAAGAGCATCAGGGGCTCGCGCCAGCCGAACTGTTCGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGGGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTQGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTCCCACAGCTC |
| | CTGGGCAACGTGCTGGTCTGTGTGCTGGCCCATCACTTTGGCAAAGGGGCCGCTGCCAAGCTTCCGAGCTCTCGAATTCATGGACATGAGGGTGCCCGCC |
| | CAGCTGCTTGGCCTGTTGCTGCTTTGGCTGCCCGGCGCCAAGTGCCAAGTGCAGCTGCAGCAGAGCGGACCCGGCCTGGTGAAGCCCAGCCAGACCCTGA |
| | GCCTGACCTGCGCCATCAGCGGCGACAGCGTGAGCTCCAACAGCGCCGCCTGGAACTGGATCAGGCAGAGCCCCAGCAGGGGCCTGGAGTGGCTGGGCA |
| | GAACCTACTACAGGTCCAAGTGGTACAACGACTACGCCGTGAGCGTGAAGAGCAGGATCACCATCAACCCCGACACCAGCAAGAACCAGTTCTCACTGCA |
| | GCTGAACAGCGTGACCCCCGAGGACACCGCCGTGTACTACTGCGCCAGGGAGGTGACCGGCGACCTGGAGGACGCCTTCGACATCTGGGGCCAGGGAAC |
| | CATGGTGACCGTGAGCAGCGGCGGAGGTGGCTCTGGCGGAGGAGGATCTGGTGGGGGAGGAAGCGGCGGTGGCGGCAGCGACATCCAGATGACCCAA |
| | AGCCCCAGCTCCCTTAGCGCCAGCGTGGGCGACAGGGTGACCATCACCTGCAGGGCCTCTCAGACCATCTGGAGCTATCTGAACTGGTACCAGCAGAGGC |
| | CCGGCAAAGCTQCCAACCTGTTGATCTACGCQGCCAGCAGCCTCCAGAGCGGCGTGCCTAGCAGGTTTAGTGGAAGGGGCAGCGGCACCGACTTCACCTT |
| | AACCATCAGCAGCCTTCAAGCCGAGGACTTTGCCACCTACTACTGCCAGCAGAGCTACAGCATCCCCCAGACCTTCGGCCAGGGCACCAAGCTGGAGATCA |
| | AGGCTAGCACCACAACACCCGCCCCCAGGCCCCCTACACCCGCTCCCACAATCGCCTCTCAGCCTCTGAGCCTTAGGCCCGAAGCTTGCAGACCTGCTGCCG |
| | GCGGCGCCGTGCATACCAGGGGACTGGACTTCGCTTGCGACATCTACATCTGGGCCCCCTTAGCCGGCACATGCGGCGTGCTGCTGCTGAGCCTGGTGATC |
| | ACCCTGTACTGCAACCACAGGAACGTGAGGAGCAAGAGGAGCAGGCTGCTGCACAGCGACTACATGAACATGACCCCCAGGCGACCCGGCCCCACCAGG |
| | AAGCACTACCAGCCCTACGCCCCACCCAGGGACTTCGCCGCCTACAGGAGCAGGGTGAAGTTCAGCAGGAGCGCCGACGCCCCCGCCTACCAGCAGGGCC |
| | AGAACCAGCTGTACAATGAACTGAACCTGGGCAGGAGGGAGGAATACGACGTTCTGGACAAGAGAAGGGGAAGGGACCCCGAGATGGGGGGCAAGCCC |
| | AGGAGGAAGAACCCCCAGGAGGGCCTGTACAACGAGCTCCAGAAGGACAAGATGGCCGAGGCCTACAGCGAGATCGGCATGAAGGGCGAGAGGAGGA |
| | GGGGCAAGGGCCACGACGGCCTGTACCAGGGCCTGAGCACCGCCACCAAGGACACCTATGACGCCCTGCACATGCAGGCCCTGCCCCCTAGGTAATGATGA |
| 85 | TCTAGAAGTTGTCTCCTCCTGCACTGACTGACTGATACAATCGATTTCTGGATCCGCAGGCCTAATCAACCTCTGGATTACAAAATTTGTGAAAGATTGACTG |
| | GTATTCTTAACTATGTTGCTCCTTTTACGCTATGTGGATACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGCACTGTGTTTGCTGACGCAACCCCCACTGGTTTGGG |
| | GCATTGCCACCACCTGTCAGCTCCTTTCCGGGACTTTCGCTTTCCCCCTCCCTATTGCCACGGCGGAACTCATCGCCGCCTGCCTTGCCCGCTGCTGGACAGG |
| | GGCTCGGCTGTTGGGCACTGACAATTCCGTGGTGTTGTCGGGGAAGCTGACGTCCTTTCCATGGCTGCTCGCCTGTGTTGCCACCTGGATTCTGCGCGGGA |
| | CGTCCTTCTGCTACGTCCCTTCGGCCCTCAATCCAGCGGACCTTCCTTCCCGCGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTACCTTCAGCTCACAGACATGATAAGATACATTGATGA |
| | GTTTGGACAAACCACAACTAGAATGCAGTGAAAAAAATGCTTTATTTGTGAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGGGAGGTTTTTTAAAGCAAGTAAAACCTCTACAAATGTGGTATTGGCCCA |
| | TCTCTATCGGTATCGTAGCATAACCCCTTGGGGCCTCTAAACGGGTCTTGAGGGGTTTTTTGTGCCCCTCGGGCCGGATTGCTATCTACCGGCATTGGCGCA |
| | GAAAAAAATGCCTGATGCGACGCTGCGCGTCTTATACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAGACATTCCTTTAATGGTCTTTTCTGGACACCACTAGGG |
| | GTCAGAAGTAGTTCATCAAACTTTCTTCCCTCCCTAATCTCATTGGTTACCTTGGGCTATCGAAACTTAATTAAGCGATCTGCATCTCAATTAGTCAGCAACCA |
| | TAGTCCCGCCCCTAACTCCGCCCATCCCGCCCCTAACTCCGCCCAGTTCCGCCCATTCTCCGCCCCATCGCTGACTAATTTTTTTTATTTATGCAGAGGCCGAG |
| | GCCGCCTCGGCCTCTGAGCTATTCCAGAAGTAGTGAGGAGGCTTTTTTGGAGGCCTAGGCTTTTGCAAAGGAGGTAGCCAACATGATTGAACAAGATGGA |
| | TTGCACGCAGGTTCTCCCGCCGCTTGGGTGGAGAGGCTATTCGGCTATGACTGGGCACAACAGACAATCGGCTGCTCTGATGCCGCCGTGTTCCGGCTGTC |
| | AGCGCAGGGGCGCCCGGTTCTTTTTGTCAAGACCGACCTGTCCGGTGCCCTGAATGAACTCCAGGACGAGGCAGCGCGGCTATCGTGGCTGGCCACGACG |
| | GGCGTTCCTTGCGCAGCTGTGCTCGACGTTGTCACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGCTACCTGCCCATTCGACCACCAAGCGAAACATCGC |
| | ATCGAGCGAGCACGTACTQGGATGGAAGCCGGTCTTGTCGATCAGGATGATCTGGACGAAGAGCATCAGGGGCTCGCGCCAGCCGAACTGTTQGCCAGG |
| | CTCAAGGCGCGGATGCCCGACGGCGAGGATCTCGTCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCTGAAGAGCTTGGCGGCGAATGGGCTGACCGCTTC |
| | CTCGTGCTTTACGGTATCGCCGCTCCCGATTCGCAGCGCATCGCCTTCTATCGCCTTCTTGACGAGTTCTTCTAGTATGTAAGCCCTGTGCCTTCTAGTTGCCA |
| | GCCATCTGTTGTTTGCCCCTCCCCCGTGCCTTCCTTGACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACAATAGCAGGCATGCTGGGGATGCGGTGGGCTC |
| | TATGGTTAATTAACCAGTCAAGTCAGCTACTTGGCGAGATCGACTTGTCTGGGTTTCGACTACGCTCAGAATTGCGTCAGTCAAGTTCGATCTGGTCCTTGC |
| | TATTGCACCCGTTCTCCGATTACGAGTTTCATTTAAATCATGTGAGCAAAAGGCCAGCAAAAGGCCAGGAACCGTAAAAAGGCCGCGTTGCTGGCGTTTTTC |
| | CATAGGCTCCGCCCCCCTGACGAGCATCACAAAAATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTT |
| | GAGTCCAACCCGGTAAGACACGACTTATCGCCACTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCC |
| | GGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTAC |
| | GGGGTCTGACGCTCAGTGGAACGAAAACTCACGTTAAGGGATTTTGGTCATGAGATTATCAAAAAGGATCTTCACCTAGATCCTTTTAAATTAAAAATGAA |
| | GTTTTAAATCAATCTAAAGTATATATGAGTAAACTTGGTCTGACAGTTACCAATGCTTAATCAGTGAGGCACCTATCTCAGCGATCTGTCTATTTCGTTCATCC |
| | ATAGTTGCATTTAAATTTCCGAACTCTCCAAGGCCCTCGTCGGAAAATCTTCAAACCTTTCGTCCGATCCATCTTGCAGGCTACCTCTCGAACGAACTATCGC |
| | AAGTCTCTTGGCCGGCCTTGCGCCTTGGCTATTGCTTGGCAGCGCCTATCGCCAGGTATTACTCCAATCCCGAATATCCGAGATCGGGATCACCCGAGAGAA |
| | GTTCAACCTACATCCTCAATCCCGATCTATCCGAGATCCGAGGAATATCGAAATCGGGGCGCGCCTGGTGTACCGAGAACGATCCTCTCAGTGCGAGTCTC |
| | GACGATCCATATCGTTGCTTGGCAGTCAGCCAGTCGGAATCCAGCTTGGGACCCAGGAAGTCCAATCGTCAGATATTGTACTCAAGCCTGGTCACGGCAGC |
| | GTACCGATCTGTTTAAACCTAGATATTGATAGTCTGATCGGTCAACGTATAATCGAGTCCTAGCTTTTGCAAACATCTATCAAGAGACAGGATCAGCAGGAG |
| | GCTTTCGCATGAGTATTCAACATTTCCGTGTCGCCCTTATTCCCTTTTTTGCGGCATTTTGCCTTCCTGTTTTTGCTCACCCAGAAACGCTGGTGAAAGTAAAA |
| | GATGCTGAAGATCAGTTGGGTGCGCGAGTGGGTTACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGTGCTGCCATAACCATGAGTGATAACACTGCGGCCA |
| | ACTTACTTCTGACAACGATTGGAGGACCGAAGGAGCTAACCGCTTTTTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAACAACCTTGCGTAAACTATTAACTGGCGAACTACTTACTCTAGCTTCC |
| | CGGCAACAGTTGATAGACTGGATGGAGGCGGATAAAGTTGCAGGACCACTTCTGCGCTCGGCCCTTCCGGCTGGCTGGTTTATTGCTGATAAATCTGGAGC |
| | CGGTGAGCGTGGGTCTCGCGGTATCATTGCAGCACTGGGGCCAGATGGTAAGCCCTCCCGTATCGTAGTTATCTACACGACGGGGAGTCAGGCAACTATG |
| | GATGAACGAAATAGACAGATCGCTGAGATAGGTGCCTCACTGATTAAGCATTGGTAACCGATTCTAGGTGCATTGGCGCAGAAAAAAATGCCTGATGCGA |
| | CGCTGCGCGTCTTATACTCCCACATATGCCAGATTCAGCAACGGATACGGCTTCCCCAACTTGCCCACTTCCATACGTGTCCTCCTTACCAGAAATTTATCCTT |
| | AAGATCCCGAATCGTTTAAACTCGACTCTGGCTCTATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATGCTATCAATCGTTGCGTTACACACACAAAAAACCAA |
| | CACACATCCATCTTCGATGGATAGCGATTTTATTATCTAACTGCTGATCGAGTGTAGCCAGATCTAGTAATCAATTACGGGGTCATTAGTTCATAGCCCATAT |
| | ATGGAGTTCCGCGTTACATAACTTACGGTAAATGGCCCGCCTGGCTGACCGCCCAACGACCCCCGCCCATTGACGTCAATAATGACGTATGTTQCCATAGTA |
| | ACGCCAATAGGGACTTTCCATTGACGTCAATGGGTGGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTCCTACTTGGCAGTACATCTACGTATTAGTCATCGCTA |
| | TTACCATGCTGATGCGGTTTTGGCAGTACATCAATGGGCGTGGATAGCGGTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCCCCATTGACGCAAATGGGGGGTAGGCGTGTACGGTGGGAGGTCTATATA |
| | AGCAGAGCTGGTTTAGTGAACCGTCAGATCGCCTGGAGACGCCATCCACGCTGTTTTGACCTCCATAGAAGACACCGGGACCGATCCAGCCTCCCCTCGAA |
| | GCTTACATGTGGTACCGAGCTCGGATCCTGAGAACTTCAGGGTGAGTCTATGGGACCCTTGATGTTTTCTTTCCCCTTCTTTTCTATGGTTAAGTTCATGTCAT |
| | AGGAAGGGGAGAAGTAACAGGGTACACATATTGACCAAATCAGGGTAATTTTGCATTTGTAATTTTAAAAAATGCTTTCTTCTTTTAATATACTTTTTTGTTT |
| | ATCTTATTTCTAATACTTTCCCTAATCTCTTTCTTTCAGGGCAATAATGATACAATGTATCATGCCTCTTTGCACCATTCTAAAGAATAACAGTGATAATTTCTG |
| | GGTTAAGGCAATAGCAATATTTCTGCATATAAATATTTCTGCATATAAATTGTAACTGATGTAAGAGGTTTCATATTGCTAATAGCAGCTACAATCCAGCTAC |
| | CATTCTGCTTTTATTTTATGGTTGGGATAAGGCTGGATTATTCTGAGTCCAAGCTAGGCCCTTTTGCTAATCATGTTCATACCTCTTATCTTCCTQCCACAGCTC |
| | CTGGGCAACGTGCTGGTCTGTGTGCTGGCCCATCACTTTGGCAAAGGCGGCCGCTGCCAAGCTTCCGAGCTCTCGAATTCATGGACATGAGGGTGCCCGCC |
| | CAGCTGCTTGGCCTGCTGCTCCTTTGGCTGCCTGGCGCCAAGTGCCAGGTGCAGCTGCAGCAGAGCGGACCCGGCCTGGTGAAGCCCAGCCAGACCCTGA |
| | GCCTGACCTGCGCCATCAGCGGCGACAGCGTGAGCAGCAACAGCGCCGCCTGGAACTGGATCAGGCAGAGCCCCAGCAGGGGCCTGGAGTGGCTGGGC |
| | AGAACCTACTACAGGTCCAAGTGGTACAACGACTACGCCGTGAGCGTGAAGAGCAGGATCACCATCAACCCCGACACCAGCAAGAACCAGTTCTCACTGC |
| | AGCTGAACAGCGTGACCCCCGAGGACACCGCCGTGTACTACTGCGCCAGGGAGGTGACCGGCGACCTGGAGGACGCCTTCGACATCTGGGGCCAGGGCA |
| | CCATGGTGACCGTGAGCTCCGGGGGGGGAGGATCTGGAGGCGGAGGTTCTGGTGGCGGGGGAAGCGGAGGCGGCGGAAGCGACATCCAGATGACCCA |
| | GAGCCCTAGCAGCCTGAGCGCCAGCGTGGGCGACAGGGTGACCATCACCTGCAGGGCCTCCCAGACCATCTGGAGCTACCTGAACTGGTACCAGCAGAG |
| | GCCCGGCAAAGCTCCCAACTTATTGATCTACGCCGCCAGCAGCCTCCAGAGCGGCGTGCCCAGCAGGTTCTCTGGCAGGGGCAGCGGCACCGACTTCACCT |
| | TAACCATCAGCAGCCTTCAGGCCGAGGACTTCGCTACCTACTACTGCCAGCAGAGCTACAGCATCCCCCAGACCTTCGGCCAAGGCACCAAGCTGGAGATC |
| | AAGGCCAGCCTTAGCAACAGCATCATGTACTTCAGCCACTTCGTGCCCGTTTTCCTGCCTGCCAAACCCACCACAACCCCTGCTCCCAGGCCCCCTACCCCCG |
| | CTCCTACCATTGCTAGCCAACCTCTGAGCCTTAGGCCCGAGGCCTGCAGACCCGCTGCCGGAGGCGCTGTGCACACAAGGGGACTGGACTTCGCATGCGAC |
| | ATCTACATCTGGGCCCCCTTAGCCGGCACCTGCGGCGTGCTGCTGCTGAGCCTGGTGATCACCCTGTACTGCAACCACAGGAACGTGAGGAGCAAGAGGA |
| | GCAGGCTGCTGCACAGCGACTACATGAACATGACCCCCAGGCGACCCGGCCCCACCAGGAAGCACTACCAGCCCTACGCCCCACCCAGGGACTTCGCCGCC |
| | TACAGGAGCAGGGTGAAGTTCAGCAGGAGCGCCGACGCCCCCGCCTACCAGCAGGGACAGAACCAGCTGTACAATGAGCTGAACCTGGGCAGGAGGGA |
| | GGAGTACGACGTGCTGGACAAGAGGAGGGGAAGGGATCCCGAGATGGGGGCAAGCCCAGGAGGAAGAACCCCCAGGAGGGCCTGTACAACGAGCTC |
| | CAGAAGGACAAGATGGCCGAGGCCTACAGCGAAATCGGCATGAAGGGCGAGAGGAGGAGGGGCAAGGGCCACGACGGCCTGTACCAGGGCCTGAGCA |
| | CCGCCACCAAGGACACATACGACGCCCTGCACATGCAGGCCCTGCCCCCTAGGTAATGATAA |
| 86 | AUGGAGACCGACACCCUCCUGCUGUGGGUGCUGCUGCUUUGGGUGCCCGGGAGCACGGGGCAGGUCCAGCUCGUGGAGAGCGGGGGGGGCGUGG |
| | UGCAGCCCGGCCGAAGCCUCCGCCUAAGCUGCGCCGCCAGCGGGUUCACCUUCAGCAGCUACGGCAUGCACUGGGUACGGCAGGCCCCCGGCAAAGG |
| | ACUGGAGUGGGUGGCGAUCAUAUACUACGACGGGAGCAAGAAAUACUACGCCGACUCCGUCAAGGGCAGGUUUACCAUCAGCCGCGACAACAGCAA |
| | GAACACGCUGUACCUGCAGAUGAACAGCCUGAGGGCCGAGGACACCGCCGUCUACUACUGUGCAAGAGAGCUAACCGGCGACGCGUUUGACAUCUG |
| | GGGGCAGGGCACCAAGGUGACCGUGUCCUCCGGGGGCGGGGGCAGCGGCGGGGGGGGCUCCGGCGGCGGCGGGUCAGGGGGGGGCGGCAGCGAGA |
| | UAGUGCUGACCCAGACCCCAUCCAGCCUGUCCGCCUCCCUGGGCGACCGCGUGACGAUCAGCUGCCGGGCCAGCCAGUCGAUCGGCAGCAGCCUACA |
| | CUGGUACCAGCAGAAGCCGGACCAGUCCCCCAAGCUGCUCAUCAAGUACGCCUCCCAGUCAUUCUCCGGGGUGCCCUCAAAGUUCAUCGGGAGCGG |
| | GUCCGGCACGGACUUCACACUCACCAUCAACAGCCUGGAGGCCGAGGACGCCGCGACCUUUUACUGCCACCAAAGCAGCACCCUCCCCUACACCUUCG |
| | GCCAGGGCACAAAGCUGGAGAUCAAGGCGGCCGCGAUAGAGGUAAUGUACCCUCCUCCUUACCUGGACAACGAGAAGUCGAACGGCACCAUCAUCC |
| | ACGUCAAGGGCAAGCACCUCUGCCCCUCGCCCCUGUUCCCCGGCCCCUCCAAGCCCUUCUGGGUGCUGGUCGUGGUUGGUGGCGUCCUCGCCUGCU |
| | ACUCCCUCCUCGUCACGGUGGCGUUCAUCAUCUUCUGGGUGCGGAGCAAGCGAAGCCGCCUCCUGCACAGCGACUACAUGAACAUGACGCCCCGCC |
| | GCCCCGGCCCGACCCGGAAGCACUACCAGCCCUACGCCCCGCCGCGCGACUUCGCGGCCUACCGCUCCCGGGUCAAGUUCUCCCGCAGCGCCGACGCC |
| | CCCGCGUACCAGCAGGGCCAGAACCAGCUCUACAACGAGCUCAACCUGGGGAGGCGCGAGGAGUACGACGUGCUUGACAAGCGCCGUGGCCGGGAC |
| | CCUGAGAUGGGGGGUAAGCCGCGACGGAAGAACCCGCAGGAGGGCCUGUACAACGAGCUGCAGAAGGACAAGAUGGCGGAGGCCUACAGCGAGAU |
| | CGGGAUGAAGGGGGAGCGGCGGCGUGGCAAGGGGCACGACGGCCUGUACCAGGGCCUCAGUACGGCCACCAAGGACACCUACGACGCCUUGCACAU |
| | GCAGGCCCUCCCCCCCCGCUAGUGAUGA |
| 87 | AUGGAGACCGACACCCUCCUGCUGUGGGUGCUGCUGCUUUGGGUGCCCGGGAGCACGGGGCAGGUGCAGCUGGUGCAGAGCGGCGCCGAGGUGA |
| | AGAAACCCGGCGCGAGCGUCAAAGUGAGCUGCAAAGCAAGCGGCUAUACAUUCACCGGCUACUACAUACACUGGGUGAGGCAAGCCCCAGGCCAGG |
| | GCCUCGAGUGGAUGGGCUGGAUCAAUCCCAACUCCGGCGGCACGAACUAUGCCCAGAAGUUCCAGGGCAGGGUGACCAUGACACGAGACACCAGCA |
| | UCGGCACCGCCUACAUGGAACUGUCAAGGCUGCGAUCCGACGACACCGCCAUUUACUACUGCACCCGGGAGGAUACCUGGAACUACAUCGACUAUU |
| | GGGGCCAGGGGACCCUCGUGACAGUCAGCUCCGGGGGGGGGGGGAGCGGCGGCGGGGGCAGGGGGGGGGGGGCUCAGGGGGGGGGGGGAGCGA |
| | CAUCCAGAUGACCCAAAGCCCCAGCAGCCUCUCCGCGAGCGUCGGGGACCGAGUGACCAUCACCUGCCGCGCCUCCCAGAGCAUCAGCUCCCAUCUCA |
| | ACUGGUACCAGCAGAAGCCCGGCAAGGCCCCCAAGCUACUGAUCUACGCCGCCUCAAGCCUCCACAGCGGCGUACCCAGCCGCUUUAGCGGGUCCGG |
| | CAGCGGCACCGACUUCACCCUCACGAUCUCCUCCCUGCAGCCCGAGGACUUCGCCACCUACUGCUGCCAGCAGAGCUACAGCACCCCCAGGACGUUCG |
| | GCCAGGGCACCAAAGUGGAAAUCAAGGCGGCCGCGAUAGAGGUAAUGUACCCUCCUCCUUACCUGGACAACGAGAAGUCGAACGGCACCAUCAUCC |
| | ACGUCAAGGGCAAGCACCUCUGCCCCUCGCCCCUGUUCCCCGGCCCCUCCAAGCCCUUCUGGGUGCUGGUCGUGGUUGGUGGCGUCCUCGCCUGCU |
| | ACUCCCUCCUCGUCACGGUGGCGUUCAUCAUCUUCUGGGUGCGGAGCAAGCGAAGCCGCCUCCUGCACAGCGACUACAUGAACAUGACGCCCCGCC |
| | GCCCCGGCCCGACCCGGAAGCACUACCAGCCCUACGCCCCGCCGCGCGACUUCGCGGCCUACCGCUCCCGGGUCAAGUUCUCCCGCAGCGCCGACGCC |
| | CCCGCGUACCAGCAGGGCCAGAACCAGCUCUACAACGAGCUCAACCUGGGGAGGCGCGAGGAGUACGACGUGCUUIGACAAGCGCCGUGGCCGGGAC |
| | CCUGAGAUGGGGGGUAAGCCGCGACGGAAGAACCCGCAGGAGGGCCUGUACAACGAGCUGCAGAAGGACAAGAUGGCGGAGGCCUACAGCGAGAU |
| | CGGGAUGAAGGGGGAGCGGCGGCGUGGCAAGGGGCACGACGGCCUGUACCAGGGCCUCAGUACGGCCACCAAGGACACCUACGACGCCUUGCACAU |
| | GCAGGCCCUCCCCCCCCGCUAGUGAUGA |
| 88 | AUGGAGACCGACACCCUCCUGCUGUGGGUGCUGCUGCUUUGGGUGCCCGGGAGCACGGGGCAGGUGCAGCUGGUGCAGAGCGGCGCCGAGGUGA |
| | AGAAACCCGGCGCGAGCGUCAAAGUGAGCUGCAAAGCAAGCGGCUAUACAUUCACCGGCUACUACAUACACUGGGUGAGGCAAGCCCCAGGCCAGG |
| | GCCUCGAGUGGAUGGGCUGGAUCAAUCCCAACUCCGGGGGCACGAACUAUGCCCAGAAGUUCCAGGGCAGGGUGACCAUGACACGAGACACCAGCA |
| | UCGGCACCGCCUACAUGGAACUGUCAAGGCUGCGAUCCGACGACACCGCCAUUUACUACUGCACCCGGGAGGAUACCUGGAACUACAUCGACUAUU |
| | GGGGCCAGGGGACCCUCGUGACAGUCAGCUCCGGGGGGGGGGGGAGCGGCGGGGGGGGCAGGGGGGGGGGGGCUCAGGGGGGGGGGGGAGCGA |
| | CAUCCAGAUGACCCAAAGCCCCAGCAGCCUCUCCGCGAGCGUCGGGGACCGAGUGACCAUCACCUGCCGCGCCUCCCAGAGCAUCAGCUCCCAUCUCA |
| | ACUGGUACCAGCAGAAGCCCGGCAAGGCCCCCAAGCUACUGAUCUACGCCGCCUCAAGCCUCCACAGCGGCGUACCCAGCCGCUUUAGCGGGUCCGG |
| | CAGCGGCACCGACUUCACCCUCACGAUCUCCUCCCUGCAGCCCGAGGACUUCGCCACCUACUGCUGCCAGCAGAGCUACAGCACCCCCAGGACGUUCG |
| | GCCAGGGCACCAAAGUGGAAAUCAAGGCGAGCACCACCACCCCCGCGCCCCGGCCGCCGACCCCCGCCCCCACAAUCGCCUCACAGCCACUGAGCCUGC |
| | GCCCCGAGGCCUGCAGGCCCGCCGCAGGCGGCGCCGUGCACACGCGUGGGCUCGACUUCGCCUGUGACAUAUACAUCUGGGCCCCCCUGGCCGGCAC |
| | CUGGGGGGUCCUGCUGCUGAGCCUGGUGAUCACCCUGUAUUGCAACCACCGGAACGUGCGCUCCAAACGCAGCCGGCUGCUGCACAGCGACUACAU |
| | GAACAUGACCCCGCGCAGGCCCGGCCCGACCCGAAAGCACUACCAGCCAUACGCCCCCCCCCGGGACUUCGCCGCCUACCGGUCAAGGGUUAAGUUCU |
| | CACGGUCGGCCGACGCCCCCGCCUACCAGCAGGGCCAGAACCAGCUGUACAAUGAGCUGAACCUGGGCCGGAGGGAGGAGUACGACGUACUGGAUA |
| | AAAGGGGGGCAGAGACCCCGAGAUGGGGGGGAAACCCAGGCGUAAGAACCCCCAGGAGGGCCUGUACAACGAACUGCAAAAGGACAAGAUGGCCG |
| | AGGCCUACAGCGAAAUAGGCAUGAAGGGCGAGCGUAGGAGGGGGAAGGGCCACGACGGCCUGUAUCAAGGCCUGUCGACCGCCACCAAAGACACCU |
| | ACGACGCCCUGCACAUGCAGGCCCUGCCCCCCAGGUAGUGAUGA |
| 89 | AUGGAGACCGACACCCUCCUGCUGUGGGUGCUGCUGCUUUGGGUGCCCGGGAGCACGGGGCAGGUGACCCUGAGGGAGAGCGGGCCCGCCCUGGU |
| | GAAGCCGACACAGACACUGACCCUCACGUGCACCUUCAGCGGCAUCAGCCUGACGACCUCCGGCAUGUGCGUGAGCUGGAUCAGGCAGCCCCCCGGG |
| | AAGGCCCUGGAGUGGCUAGCCCUCAUCGACUGGGACGGGGACAACUACUACUCCACCUCACUCAAGACAAGGCUGACCAUAAGCAAGGACACCAGCA |
| | AGAACCAGGUCGUGCUGACCAUGGCCAGCAUGGAUCCGGUGGAUACCGCCACCUACUAUUGCGCCCGGAUGGGAUACACCUACGGCUACGACGCCU |
| | UUGACAUCUGGGGGCAGGGCACAAUGGUGACGGUCUCGUCCGGAGGGGGGGGCUCCGGAGGGGGAGGCAGGGGGGGGGGGGAUCGGGGGGGGG |
| | CGGCUCCGAGAUCGUCCUCACCCAAACCCCCUCAAGCCUGAGCGCAAGCCUGGGCGACCGCGUGACCAUCUCCUGCAGGGCCAGCCAAUCAAUCGGC |
| | AGCCUGCUCCACUGGUACCAGCAGAAACCCGACCAGUCCCCAAAGCUGCUGAUCAAGUUCGCCAGCCAGUCACUGAGCGGCGUGCCCAGCCGCUUCA |
| | GCGGCAGCGGCUCCGGCACCGACUUCACCCUGACAAUAAACUCCCUCGAAGCCGAGGACGCCGCCACCUACUACUGCCACCAGAGCAGGAGCCUGCCC |
| | UUCACCUUCGGACCCGGCACCAAGGUGGACAUCAACGCAAGCAGCCUGAGGCCGGAGGCAUGCCGGCCAGCAGCCGGGGGCGCGGUACACACCQGCG |
| | GCCUGGACUUCGCGUGUGACAUCUACAUCUGGGCCCCCCUGGCCGGCACGUGCGGCGUCCUGCUGCUGAGCCUCGUGAUCACACUGUACUGCAACC |
| | ACCGGAACGUCAGGUCCAAGAGGAGCCGCCUGCUCCACAGCGACUACAUGAACAUGACCCCGAGGCGACCCGGCCCCACGAGGAAACACUACCAGCCC |
| | UACGCCCCACCCAGGGACUUCGCGGCCUAUCGAAGCCGGGUGAAGUUCUCACGCAGCGCCGAUGCCCCCGCCUACCAGCAGGGGCAGAACCAGCUCU |
| | ACAACGAACUGAACCUGGGACGACGGGAGGAGUACGACGUACUGGAUAAGCGGCGUGGGCGUGACCCCGAAAUGGGGGGGAAGCCCCGGAGGAAG |
| | AACCCCCAGGAGGGACUGUACAACGAGCUGCAGAAGGACAAGAUGGCGGAGGCGUACAGCGAGAUCGGCAUGAAGGGCGAGCGAAGGCGUGGCAA |
| | GGGGCACGACGGCCUGUAUCAGGGGCUGUCAACCGCAACCAAGGACACAUACGACGCCCUCCACAUGCAAGCCCUGCCCCCCCGCUAGUGAUGA |
| 90 | AUGGAGACCGACACCCUCCUGCUGUGGGUGCUGCUGCUUUGGGUGCCCGGGAGCACGGGGCAGGUGACCCUGAGGGAGAGCGGGCCCGCCCUGGU |
| | GAAGCCGACACAGACACUGACCCUCACGUGCACCUUCAGCGGCAUCAGCCUGACGACCUCCGGCAUGUGCGUGAGCUGGAUCAGGCAGCCCCCCGGG |
| | AAGGCCCUGGAGUGGCUAGCCCUCAUCGACUGGGACGGGGACAACUACUACUCCACCUCACUCAAGACAAGGCUGACCAUAAGCAAGGACACCAGCA |
| | AGAACCAGGUCGUGCUGACCAUGGCCAGCAUGGAUCCGGUGGAUACCGCCACCUACUAUUGCGCCCGGAUGGGAUACACCUACGGCUACGACGCCU |
| | UUGACAUCUGGGGGCAGGGCACAAUGGUGACGGUCUCGUCCGGAGGGGGGGGCUCCGGAGGGGGAGGCAGCGGCGGCGGCGGAUCGGGGGGGGG |
| | CGGCUCCGAGAUCGUCCUCACCCAAACCCCCUCAAGCCUGAGCGCAAGCCUGGGCGACCGCGUGACCAUCUCCUGCAGGGCCAGCCAAUCAAUCGGC |
| | AGCCUGCUCCACUGGUACCAGCAGAAACCCGACCAGUCCCCAAAGCUGCUGAUCAAGUUCGCCAGCCAGUCACUGAGCGGCGUGCCCAGCCGCUUCA |
| | GCGGCAGCGGCUCCGGCACCGACUUCACCCUGACAAUAAACUCCCUCGAAGCCGAGGACGCCGCCACCUACUACUGCCACCAGAGCAGGAGCCUGCCC |
| | UUCACCUUCGGACCCGGCACCAAGGUGGACAUCAACGCAAGCACCACCACCCCCGCCCCCCGCCCCCCCACACCCGCCCCCACCAUCGCAAGCCAGCCCC |
| | UGAGCCUGAGGCCGGAGGCAUGCCGGCCAGCAGCCGGGGGCGCGGUACACACCCGCGGCCUGGACUUCGCGUGUGACAUCUACAUCUGGGCCCCCC |
| | UGGCCGGCACGUGCGGCGUCCUGCUGCUGAGCCUCGUGAUCACACUGUACUGCAACCACCGGAACGUCAGGUCCAAGAGGAGCCGCCUGCUCCACA |
| | GCGACUACAUGAACAUGACCCCGAGGCGACCCGGCCCCACGAGGAAACACUACCAGCCCUACGCCCCACCCAGGGACUUCGCGGCCUAUCGAAGCCGG |
| | GUGAAGUUCUCACGCAGCGCCGAUGCCCCCGCCUACCAGCAGGGGCAGAACCAGCUCUACAACGAACUGAACCUGGGACGACGGGAGGAGUACGAC |
| | GUACUGGAUAAGCGGCGUGGGCGUGACCCCGAAAUGGGGGGGAAGCCCCGGAGGAAGAACCCCCAGGAGGGACUGUACAACGAGCUGCAGAAGGA |
| | CAAGAUGGCGGAGGCGUACAGCGAGAUCGGCAUGAAGGGCGAGCGAAGGCGUGGCAAGGGGCACGACGGCCUGUAUCAGGGGCUGUCAACCGCAA |
| | CCAAGGACACAUACGACGCCCUCCACAUGCAAGCCCUGCCCCCCCGCUAGUGAUGA |
| 91 | AUGGCUCUCCCAGUGACCGCCCUGCUGCUGCCCCUAGCCCUCCUUUUGCACGCAGCGAGGCCUCAGGUGACCCUGAGGGAGAGCGGGCCCGCCCUG |
| | GUGAAGCCGACACAGACACUGACCCUCACGUGCACCULICAGCGGCAUCAGCCUGACGACCUCCGGCAUGUGCGUGAGCUGGAUCAGGCAGCCCCCCG |
| | GGAAGGCCCUGGAGUGGCUAGCCCUCAUCGACUGGGACGGGGACAACUACUACUCCACCUCACUCAAGACAAGGCUGACCAUAAGCAAGGACACCA |
| | GCAAGAACCAGGUCGUGCUGACCAUGGCCAGCAUGGAUCCGGUGGAUACCGCCACCUACUAUUGCGCCCGGAUGGGAUACACCUACGGCUACGACG |
| | CCUUUGACAUCUGGGGGCAGGGCACAAUGGUGACGGUCUCGUCCGGAGGGGGGGGCUCCGGAGGGGGAGGCAGGGGGGGGGGGGAUCGGGGGG |
| | GGGCGGCUCCGAGAUCGUCCUCACCCAAACCCCCUCAAGCCUGAGCGCAAGCCUGGGCGACCGCGUGACCAUCUCCUGCAGGGCCAGCCAAUCAAUC |
| | GGCAGCCUGCUCCACUGGUACCAGCAGAAACCCGACCAGUCCCCAAAGCUGCUGAUCAAGUUCGCCAGCCAGUCACUGAGCGGCGUGCCCAGCCGCU |
| | UCAGCGGCAGCGGCUCCGGCACCGACUUCACCCUGACAAUAAACUCCCUCGAAGCCGAGGACGCCGCCACCUACUACUGCCACCAGAGCAGGAGCCU |
| | GCCCUUCACCUUCGGACCCGGCACCAAGGUGGACAUCAACGCAAGCACCACCACCCCCGCCCCCCGCCCCCCCACACCCGCCCCCACCAUCGCAAGCCAG |
| | CCCCUGAGCCUGAGGCCGGAGGCAUGCCGGCCAGCAGCCGGGGGCGCGGUACACACCCGCGGCCUGGACUUCGCGUGUGACAUCUACAUCUGGGCC |
| | CCCCUGGCCGGCACGUGCGGCGUCCUGCUGCUGAGCCUCGUGAUCACACUGUACUGCAACCACCGGAACGUCAGGUCCAAGAGGAGCCGCCUGCUC |
| | CACAGCGACUACAUGAACAUGACCCCGAGGCGACCCGGCCCCACGAGGAAACACUACCAGCCCUACGCCCCACCCAGGGACUUCGQGGCCUAUCGAAG |
| | CCGGGUGAAGUUCUCACGCAGCGCCGAUGCCCCCGCCUACCAGCAGGGGCAGAACCAGCUCUACAACGAACUGAACCUGGGACGACGGGAGGAGUA |
| | CGACGUACUGGAUAAGCGGCGUGGGCGUGACCCCGAAAUGGGGGGGAAGCCCCGGAGGAAGAACCCCCAGGAGGGACUGUACAACGAGCUGCAGA |
| | AGGACAAGAUGGCGGAGGCGUACAGCGAGAUCGGCAUGAAGGGCGAGCGAAGGCGUGGCAAGGGGCACGACGGCCUGUAUCAGGGGCUGUCAACC |
| | GCAACCAAGGACACAUACGACGCCCUCCACAUGCAAGCCCUGCCCCCCCGCUAGUGAUGA |
| 92 | AUGGAGACCGACACCCUCCUGCUGUGGGUGCUGCUGCUUUGGGUGCCCGGGAGCACGGGGCAAGUCACCCUGCGGGAGAGCGGCCCCGCCCUGGU |
| | CAAGACCACGCAGACCCUGACCCUCACCUGCACCUUCAGCGGCUUUAGCCUCAGCACCAGCGGCAUGUGCGUGAGCUGGAUCCGCCAGCCCCCCGGG |
| | AAGGCACUGGAGUGGCUCGCCCUGAUCGACUGGGAUGAUGACAAAUACUACAGCACCAGCCUGAAGACCCGCCUGACGAUCUCAAAGGAUACCAGC |
| | AAGAGCCAGGUGGUCCUGACCAUGACCAGCAUGGACCCCGUCGACACCGCCACCUACUACUGCGCCCGCAUCAGGUACAUCUACGGAUACGACUACU |
| | UCGAUUACUGGGGCCAGGGAACCCUGGUGACCGUGUCGAGCGGGGGGGGAGGGAGCGGCGGCGGGGGCAGCGGCGGCGGCGGGUCCGGCGGGGG |
| | GGGCUCCGACAUCCAGAUGACCCAGAGCCCCAGCAGCCUCUCCGCCAGCGUGGGGGAUCGUGUGACCAUCACCUGUCGCGCGUCCCAACGAAUAAGC |
| | AGCCAGCUCAAUUGGUACCAGCAGAAGCCAGGGAAAGCACCCAAGCUGCUGAUCUACGCAGCCAGCAGCCUGCAGAGCGGCGUCCCCUCACGCUUCU |
| | CAGGCAGCGGCUCCGGAACAGAGUUCACGCUGACAAUCAGCUCCCUGCAGCCAGAGGACAUAGCGACCUAUUGCUGCCAACAGACCUACAGCAAGCC |
| | CAUCACGUUCGGCCAAGGAACCCGGCUGGAGAUCAAGGCGGCCGCGAUAGAGGUAAUGUACCCUCCUCCUUACCUGGACAACGAGAAGUCGAACGG |
| | CACCAUCAUCCACGUCAAGGGCAAGCACCUCUGCCCCUCGCCCCUGUUCCCCGGCCCCUCCAAGCCCUUCUGGGUGCUGGUCGUGGUUGGUGGCGU |
| | CCUCGCCUGCUACUCCCUCCUCGUCACGGUGGCGUUCAUCAUCUUCUGGGUGCGGAGCAAGCGAAGCCGCCUCCUGCACAGCGACUACAUGAACAU |
| | GACGCCCCGCCGCCCCGGCCCGACCCGGAAGCACUACCAGCCCUACGCCCCGCCGCGCGACUUCGCGGCCUACCGCUCCCGGGUCAAGUUCUCCCGCA |
| | GCGCCGACGCCCCCGCGUACCAGCAGGGCCAGAACCAGCUCUACAACGAGCUCAACCUGGGGAGGCGCGAGGAGUACGACGUGCUUGACAAGCGCCG |
| | UGGCCGGGACCCUGAGAUGGGGGGUAAGCCGCGACGGAAGAACCCGCAGGAGGGCCUGUACAACGAGCUGCAGAAGGACAAGAUGGCGGAGGCCU |
| | ACAGCGAGAUCGGGAUGAAGGGGGAGCGGCGGCGUGGCAAGGGGCACGACGGCCUGUACCAGGGCCUCAGUACGGCCACCAAGGACACCUACGACG |
| | CCUUGCACAUGCAGGCCCUCCCCCCCCGCUAGUGAUGA |
| 93 | AUGGAGACCGACACCCUCCUGCUGUGGGUGCUGCUGCUUUGGGUGCCCGGGAGCACGGGGGAGGUGCAGCUGGUGGAGAGCGGCGGCGGCCUGG |
| | UGCAGCCCGGCAGGAGCCUGAGGCUGAGCUGCGCCGCCAGCGGCUUCACCULICGACGGCUACGCCAUGCACUGGGUGAGGCAGGCCCCCGGCAAGG |
| | GCCUGGAGUGGGUGAGCGGCAUCAGCUGGAACAGCUACAGCCUGGGCUACGCCGACAGCGUGAAGGGCAGGUUCACCAUCAGCAGGGACAACGCC |
| | AAGAACAGCCUGUACCUGCAGAUGAACAGCCUGAGGAUCGAGGACACCGCCCUGUACUACUGCGCCAAGGAGUUCGGCUGGGACCUGCCCUACUUC |
| | GACUACUGGGGCCAGGGCACCCUGGUGACCGUGAGCAGCGGCGGGGGGGGCAGCGGCGGCGGCGGCAGCGGGGGGGGCGGCAGCGGCGGGGGGGG |
| | CAGCGACAUCGUGAUGACCCAGAGCCCCGACAGCCUGGCCGUGAGCCUGGGCGAGAGGGCCACCAUCGACUGCAAGAGCAGCCAGAACGUGCUGUA |
| | CAGCAGCAGCAACAAGAACUACCUGGCCUGGUACCAGCAGAAGCCCGGCCAGCCCCCCAACCUGCUGAUCUACUGGGCCAGCACCAGGGAGAGCGGC |
| | GUGCCCGACAGGUUCAGCGGCAGCGGCAGCGGCACCGACUUCACCCUGACCAUCAGCAGCCUGCAGGCCGAGGACGUGGCCGUGUACUACUGCCAG |
| | CAGUACUACAGCAUCCCCUACACCUUCGGCCAGGGCACCAAGCUGGAGAUCAAGGCGGCCGCGAUAGAGGUAAUGUACCCUCCUCCUUACCUGGAC |
| | AACGAGAAGUCGAACGGCACCAUCAUCCACGUCAAGGGCAAGCACCUCUGCCCCUCGCCCCUGUUCCCCGGCCCCUCCAAGCCCUUCUGGGUGCUGG |
| | UCGUGGUUGGUGGCGUCCUCGCCUGCUACUCCCUCCUCGUCACGGUGGCGUUCAUCAUCUUCUGGGUGCGGAGCAAGCGAAGCCGCCUCCUGCAC |
| | AGCGACUACAUGAACAUGACGCCCCGCCGCCCCGGCCCGACCCGGAAGCACUACCAGCCCUACGCCCCGCCGCGCGACUUCGCGGCCUACCGCUCCCG |
| | GGUCAAGUUCUCCCGCAGCGCCGACGCCCCCGCGUACCAGCAGGGCCAGAACCAGCUCUACAACGAGCUCAACCUGGGGAGGCGCGAGGAGUACGAC |
| | GUGCUUGACAAGCGCCGUGGCCGGGACCCUGAGAUGGGGGGUAAGCCGCGACGGAAGAACCCGCAGGAGGGCCUGUACAACGAGCUGCAGAAGGA |
| | CAAGAUGGCGGAGGCCUACAGCGAGAUCGGGAUGAAGGGGGAGCGGCGGCGUGGCAAGGGGCACGACGGCCUGUACCAGGGCCUCAGUACGGCCA |
| | CCAAGGACACCUACGACGCCUUGCACAUGCAGGCCCUCCCCCCCCGCUAGUGAUGA |
| 94 | AUGGAGACCGACACCCUCCUGCUGUGGGUGCUGCUGCUUUGGGUGCCCGGGAGCACGGGGCAGGUGCAGCUGCAGCAGUCCGGCCCAGGGCUGGU |
| | GAAGCCAUCCCAGACCCUGAGCCUGACCUGCGCCAUCUCAGGCGACAGCGUCUCCAGCAACUCCGCCGCCUGGAAUUGGAUCCGGCAGAGCCCCAGC |
| | AGGGGCCUCGAGUGGCUGGGGCGUACCUACUACCGCAGCAAGUGGUACAACGACUACGCGGUGAGCGUGAAGUCAAGGAUCACCAUCAACCCCGAU |
| | ACAAGCAAGAACCAAUUCUCACUGCAGCUGAACUCCGUGACGCCCGAGGACACAGCCGUGUACUACUGCGCCCGGGAGGUGACCGGCGACCUGGAA |
| | GACGCCUUCGACAUCUGGGGACAGGGCACAAUGGUGACGGUCAGCUCCGGCGGGGGGGGGUCGGGGGGGGGGGGAGCGGAGGCGGCGGCAGCG |
| | GCGGCGGCGGAAGCGACAUCCAGAUGACGCAGAGCCCCAGCAGCCUGAGCGCCAGCGUAGGAGACCGCGUCACCAUCACCUGCAGGGCAAGCCAGAC |
| | CAUCUGGAGCUACCUGAACUGGUACCAGCAGCGUCCCGGCAAGGCCCCCAAUCUGCUGAUCUACGCCGCCUCCAGCCUGCAGUCCGGAGUGCCCUCG |
| | AGGUUCAGCGGCCGGGGCAGCGGGACCGACUUCACCCUGACCAUCAGCUCGCUGCAGGCCGAGGACUUUGCCACCUACUACUGCCAACAGAGCUAC |
| | UCAAUCCCCCAGACGUUUGGCCAGGGCACCAAGCUGGAGAUCAAGGCCAGCACCACAACCCCCGCGCCCCGGCCCCCCACCCCCGCCCCCACCAUCGCC |
| | AGCCAGCCGCUGAGCCUGAGGCCGGAAGCGUGCCGACCGGCCGCCGGGGGCGCCGUACACACCCGCGGACUCGACUUCGCCUGCGACAUAUAUAUC |
| | UGGGCCCCCCUGGCCGGCACCUGCGGCGUCCUGCUCCUGAGCCUGGUGAUCACCCUGUACUGCAACCACAGGAACGUCCGGUCCAAGCGUUCCCGCC |
| | UCCUGCACAGCGAUUACAUGAACAUGACCCCCCGACGCCCCGGCCCAACCCGAAAGCACUACCAGCCAUAUGCCCCCCCCAGGGACUUUGCCGCCUAU |
| | CGAUCCAGGGUGAAGUUCAGCAGGUCAGCGGACGCGCCCGCCUACCAGCAGGGCCAGAACCAGCUGUAUAACGAGCUGAACCUCGGGGGGCGAGAG |
| | GAGUACGACGUGCUCGACAAGCGCCGGGGCAGGGACCCCGAAAUGGGAGGGAAGCCCCGCAGGAAAAACCCCCAGGAGGGACUCUACAACGAGCUC |
| | CAAAAGGAUAAGAUGGCCGAAGCCUACAGCGAGAUCGGCAUGAAAGGGGAGAGGAGGCGGGGCAAGGGGCACGACGGCCUGUACCAGGGACUGUC |
| | AACCGCCACCAAGGACACCUACGACGCCCUGCACAUGCAGGCCCUGCCGCCCAGGUAGUGAUGA |
| 95 | AUGGAGACCGACACCCUCCUGCUGUGGGUGCUGCUGCUUUGGGUGCCCGGGAGCACGGGGCAGGUGCAGCUGCAGCAGUCCGGCCCAGGGCUGGU |
| | GAAGCCAUCCCAGACCCUGAGCCUGACCUGCGCCAUCUCAGGCGACAGCGUCUCCAGCAACUCCGCCGCCUGGAAUUGGAUCCGGCAGAGCCCCAGC |
| | AGGGGCCUCGAGUGGCUGGGGCGUACCUACUACCGCAGCAAGUGGUACAACGACUACGCGGUGAGCGUGAAGUCAAGGAUCACCAUCAACCCCGAU |
| | ACAAGCAAGAACCAAUUCUCACUGCAGCUGAACUCCGUGACGCCCGAGGACACAGCCGUGUACUACUGCGCCCGGGAGGUGACCGGCGACCUGGAA |
| | GACGCCUUCGACAUCUGGGGACAGGGCACAAUGGUGACGGUCAGCUCCGGGGGGGGGGGGUCGGGGGGGGGGGGAGCGGAGGCGGCGGCAGCG |
| | GCGGCGGCGGAAGCGACAUCCAGAUGACGCAGAGCCCCAGCAGCCUGAGCGCCAGCGUAGGAGACCGCGUCACCAUCACCUGCAGGGCAAGCCAGAC |
| | CAUCUGGAGCUACCUGAACUGGUACCAGCAGCGUCCCGGCAAGGCCCCCAAUCUGCUGAUCUACGCCGCCUCCAGCCUGCAGUCCGGAGUGCCCUCG |
| | AGGUUCAGCGGCCGGGGCAGCGGGACCGACUUCACCCUGACCAUCAGCUCGCUGCAGGCCGAGGACUUUGCCACCUACUACUGCCAACAGAGCUAC |
| | UCAAUCCCCCAGACGUUUGGCCAGGGCACCAAGCUGGAGAUCAAGGCCAGCCUUAGCAACAGCAUCAUGUACUUCAGCCACUUUGUCCCCGUCUUC |
| | CUGCCAGCCAAGCCGACCACAACCCCCGCGCCCCGGCCCCCCACCCCCGCCCCCACCAUCGCCAGCCAGCCGCUGAGCCUGAGGCCGGAAGCGUGCCGA |
| | CCGGCCGCCGGGGGCGCCGUACACACCCGCGGACUCGACUUCGCCUGCGACAUAUAUAUCUGGGCCCCCCUGGCCGGCACCUGCGGCGUCCUGCUCC |
| | UGAGCCUGGUGAUCACCCUGUACUGCAACCACAGGAACGUCCGGUCCAAGCGUUCCCGCCUCCUGCACAGCGAUUACAUGAACAUGACCCCCCGACG |
| | CCCCGGCCCAACCCGAAAGCACUACCAGCCAUAUGCCCCCCCCAGGGACUUUGCCGCCUAUCGAUCCAGGGUGAAGUUCAGCAGGUCAGCGGACGCG |
| | CCCGCCUACCAGCAGGGCCAGAACCAGCUGUAUAACGAGCUGAACCUCGGGGGGCGAGAGGAGUACGACGUGCUCGACAAGCGCCGGGGCAGGGAC |
| | CCCGAAAUGGGAGGGAAGCCCCGCAGGAAAAACCCCCAGGAGGGACUCUACAACGAGCUCCAAAAGGAUAAGAUGGCCGAAGCCUACAGCGAGAUC |
| | GGCAUGAAAGGGGAGAGGAGGGGGGCAAGGGGCACGACGGCCUGUACCAGGGACUGUCAACCGCCACCAAGGACACCUACGACGCCCUGCACAUG |
| | CAGGCCCUGCCGCCCAGGUAGUGAUGA |
| 96 | AUGGAGACCGACACCCUCCUGCUGUGGGUGCUGCUGCUUUGGGUGCCCGGGAGCACGGGGCAGGUGCAGCUGCAGCAGUCCGGCCCAGGGCUGGU |
| | GAAGCCAUCCCAGACCCUGAGCCUGACCUGCGCCAUCUCAGGCGACAGCGUCUCCAGCAACUCCGCCGCCUGGAAUUGGAUCCGGCAGAGCCCCAGC |
| | AGGGGCCUCGAGUGGCUGGGGCGUACCUACUACCGCAGCAAGUGGUACAACGACUACGCGGUGAGCGUGAAGUCAAGGAUCACCAUCAACCCCGAU |
| | ACAAGCAAGAACCAAUUCUCACUGCAGCUGAACUCCGUGACGCCCGAGGACACAGCCGUGUACUACUGCGCCCGGGAGGUGACCGGCGACCUGGAA |
| | GACGCCUUCGACAUCUGGGGACAGGGCACAAUGGUGACGGUCAGCUCCGGGGGGGGGGGGUCGGACAUCCAGAUGACGCAGAGCCCCAGCAGCCU |
| | GAGCGCCAGCGUAGGAGACCGCGUCACCAUCACCUGCAGGGCAAGCCAGACCAUCUGGAGCUACCUGAACUIGGUACCAGCAGCGUCCCGGCAAGGCC |
| | CCCAAUCUGCUGAUCUACGCCGCCUCCAGCCUGCAGUCCGGAGUGCCCUCGAGGUUCAGCGGCCGGGGCAGCGGGACCGACUUCACCCUGACCAUCA |
| | GCUCGCUGCAGGCCGAGGACUUUGCCACCUACUACUGCCAACAGAGCUACUCAAUCCCCCAGACGUUUGGCCAGGGCACCAAGCUGGAGAUCAAGG |
| | CCAGCACCACAACCCCCGCGCCCCGGCCCCCCACCCCCGCCCCCACCAUCGCCAGCCAGCCGCUGAGCCUGAGGCCGGAAGCGUGCCGACCGGCCGCCG |
| | GGGGCGCCGUACACACCCGCGGACUCGACUUCGCCUGCGACAUAUAUAUCUGGGCCCCCCUGGCCGGCACCUGCGGCGUCCUGCUCCUGAGCCUGG |
| | UGAUCACCCUGUACUGCAACCACAGGAACGUCCGGUCCAAGCGUUCCCGCCUCCUGCACAGCGAUUACAUGAACAUGACCCCCCGACGCCCCGGCCCA |
| | ACCCGAAAGCACUACCAGCCAUAUGCCCCCCCCAGGGACUUUGCCGCCUAUCGAUCCAGGGUGAAGUUCAGCAGGUCAGCGGACGCGCCCGCCUACC |
| | AGCAGGGCCAGAACCAGCUGUAUAACGAGCUGAACCUCGGGGGGCGAGAGGAGUACGACGUGCUCGACAAGCGCCGGGGCAGGGACCCCGAAAUGG |
| | GAGGGAAGCCCCGCAGGAAAAACCCCCAGGAGGGACUCUACAACGAGCUCCAAAAGGAUAAGAUGGCCGAAGCCUACAGCGAGAUCGGCAUGAAAG |
| | GGGAGAGGAGGCGGGGCAAGGGGCACGACGGCCUGUACCAGGGACUGUCAACCGCCACCAAGGACACCUACGACGCCCUGCACAUGCAGGCCCUGC |
| | CGCCCAGGUAGUGAUGA |
| 97 | METDTLLLWVLLLWVPGSTGQVQLVESGGGVVQPGRSLRLSCAASGFTFSSYGMHWVRQA |
| | PGKGLEWVAHIYYDGSKKYYADSVKGRFTISRDNSKNTLYLQMNSLRAEDTAVYYCAREL |
| | TGDAFDIWGQGTKVTVSSGGGGSGGGGSGGGGSGGGGSEIVLTQTPSSLSASLGDRVTIS |
| | CRASQSIGSSLHWYQQKPDQSPKLLIKYASQSFSGVPSKFIGSGSGTDFTLTINSLEAED |
| | AATFYCHQSSTLPYTFGQGTKLEIKAAAIEVMYPPPYLDNEKSNGTIIHVKGKHLCPSPL |
| | FPGPSKPFWVLVVVGGVLACYSLLVTVAFIIFWVRSKRSRLLHSDYMNMTPRRPGPTRKH |
| | YQPYAPPRDFAAYRSRVKESRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTYDALH |
| | MQALPPR*** |
| 98 | METDTLLLWVLLLWVPGSTGQVQLVQSGAEVKKPGASVKVSCKASGYTFTGYYIHWVRQA |
| | PGQGLEWMGWINPNSGGTNYAQKFQGRVTMTRDTSIGTAYMELSRLRSDDTAIYYCTRED |
| | TWNYIDYWGQGTLVTVSSGGGGSGGGGSGGGGSGGGGSDIQMTQSPSSLSASVGDRVTIT |
| | CRASQSISSHINWYQQKPGKAPKLLIYAASSLHSGVPSRFSGSGSGTDFTLTISSLQPED |
| | FATYCCQQSYSTPRTFGQGTKVEIKAAAIEVMYPPPYLDNEKSNGTIIHVKGKHLCPSPL |
| | FPGPSKPFWVLVVVGGVLACYSLLVTVAFIIFWVRSKRSRLLHSDYMNMTPRRPGPTRKH |
| | YQPYAPPRDFAAYRSRVKFSRSADAPAYQQGQNQLYNELNLGRREEYDVLDKRRGRDPEM |
| | GGKPRRKNPQEGLYNELQKDKMAEAYSEIGMKGERRRGKGHDGLYQGLSTATKDTYDALH |
| | MQALPPR*** |
| 99 | METDTLLLWVLLLWVPGSTGQVQLVQSGAEVKKPGASVKVSCKASGYTFTGYYIHWVRQA |
| | PGQGLEWMGWINPNSGGTNYAQKFQGRVTMTRDTSIGTAYMELSRLRSDDTAIYYCTRED |
| | TWNYIDYWGQGTLVTVSSGGGGSGGGGSGGGGSGGGGSDIQMTQSPSSLSASVGDRVTIT |
| | CRASQSISSHLNWYQQKPGKAPKLLIYAASSLHSGVPSRFSGSGSGTDFTLTISSLQPED |
| | FATYCCQQSYSTPRTFGQGTKVEIKASTTTPAPRPPTPAPTIASQPLSLRPEACRPAAGG |
| | AVHTRGLDFACDIYIWAPLAGTCGVLLLSLVITLYCNHRNVRSKRSRLLHSDYMNMTPRR |
| | PGPTRKHYQPYAPPRDFAAYRSRVKFSRSADAPAYQQGQNQLYNELNLGRREEYDVLDKR |
| | RGRDPEMGGKPRRKNPQEGLYNELQKDKMAEAYSEIGMKGERRRGKGHDGLYQGLSTATK |
| | DTYDALHMQALPPR*** |
| 100 | METDTLLLWVLLLWVPGSTGQVTLRESGPALVKPTQTLTLTCTFSGISLTTSGMCVSWIR |
| | QPPGKALEWLALIDWDGDNYYSTSLKTRLTISKDTSKNQVVLTMASMDPVDTATYYCARM |
| | GYTYGYDAFDIWGQGTMVTVSSGGGGSGGGGSGGGGSGGGGSEIVLTQTPSSLSASLGDR |
| | VTISCRASQSIGSLLHWYQQKPDQSPKLLIKFASQSLSGVPSRFSGSGSGTDFTLTINSL |
| | EAEDAATYYCHQSRSLPFTFGPGTKVDINASSLRPEACRPAAGGAVHTRGLDFACDIYIW |
| | APLAGTCGVLLLSLVITLYCNHRNVRSKRSRLLHSDYMNMTPRRPGPTRKHYQPYAPPRD |
| | FAAYRSRVKFSRSADAPAYQQGQNQLYNELNLGRREEYDVLDKRRGRDPEMGGKPRRKNP |
| | QEGLYNELQKDKMAEAYSEIGMKGERRRGKGHDGLYQGLSTATKDTYDALHMQALPPR*** |
| 101 | METDTLLLWVLLLWVPGSTGQVTLRESGPALVKPTQTLTLTCTFSGISLTTSGMCVSWIR |
| | QPPGKALEWLALIDWDGDNYYSTSLKTRLTISKDTSKNQVVLTMASMDPVDTATYYCARM |
| | GYTYGYDAFDIWGQGTMVTVSSGGGGSGGGGSGGGGGGGGSEIVLTQTPSSLSASLGDR |
| | VTISCRASQSIGSLLHWYQQKPDQSPKLLIKFASQSLSGVPSRFSGSGSGTDFTLTINSL |
| | EAEDAATYYCHQSRSLPFTFGPGTKVDINASTTTPAPRPPTPAPTIASQPLSLRPEACRP |
| | AAGGAVHTRGLDFACDIYIWAPLAGTCGVLLLSLVITLYCNHRNVRSKRSRLLHSDYMNM |
| | TPRRPGPTRKHYQPYAPPRDFAAYRSRVKFSRSADAPAYQQGQNQLYNELNLGRREEYDV |
| | LDKRRGRDPEMGGKPRRKNPQEGLYNELQKDKMAEAYSEIGMKGERRRGKGHDGLYQGLS |
| | TATKDTYDALHMQALPPR*** |
| 102 | MALPVTALLLPLALLLHAARPQVTLRESGPALVKPTQTLTLTCTESGISLTTSGMCVSWI |
| | RQPPGKALEWLALIDWDGDNYYSTSLKTRLTISKDTSKNQVVLTMASMDPVDTATYYCAR |
| | MGYTYGYDAFDIWGQGTMVTVSSGGGGSGGGGSGGGGSGGGGSEIVLTQTPSSLSASLGD |
| | RVTISCRASQSIGSLLHWYQQKPDQSPKLLIKFASQSLSGVPSRESGSGSGTDFTLTINS |
| | LEAEDAATYYCHQSRSLPFTFGPGTKVDINASTTTPAPRPPTPAPTIASQPLSLRPEACR |
| | PAAGGAVHTRGLDFACDIYIWAPLAGTCGVLLLSLVITLYCNHRNVRSKRSRLLHSDYMN |
| | MTPRRPGPTRKHYQPYAPPRDFAAYRSRVKFSRSADAPAYQQGQNQLYNELNLGRREEYD |
| | VLDKRRGRDPEMGGKPRRKNPQEGLYNELQKDKMAEAYSEIGMKGERRRGKGHDGLYQGL |
| | STATKDTYDALHMQALPPR*** |
| 103 | METDTLLLWVILLWVPGSTGQVTIRESGPALVKTTQTLTLTCTFSGFSLSTSGMCVSWIR |
| | QPPGKALEWLALIDWDDDKYYSTSLKTRLTISKDTSKSQVVLTMTSMDPVDTATYYCARI |
| | RYIYGYDYFDYWGQGTLVTVSSGGGGSGGGGSGGGGSGGGGSDIQMTQSPSSLSASVGDR |
| | VTITCRASQRISSQLNWYQQKPGKAPKLLIYAASSLQSGVPSRFSGSGSGTEFTLTISSL |
| | QPEDIATYCCQQTYSKPITFGQGTRLEIKAAAIEVMYPPPYLDNEKSNGTHIHVKGKHLC |
| | PSPLFPGPSKPFWVLVVVGGVLACYSLLVTVAFIIFWVRSKRSRLLHSDYMNMTPRRPGP |
| | TRKHYQPYAPPRDFAAYRSRVKFSRSADAPAYQQGQNQLYNELNLGRREEYDVLDKRRGR |
| | DPEMGGKPRRKNPQEGLYNELQKDKMAEAYSEIGMKGERRRGKGHDGLYQGLSTATKDTY |
| | DALHMQALPPR*** |
| 104 | METDTELLWVILLWVPGSTGEVQLVESGGGLVQPGRSLRLSCAASGFTFDGYAMHWVRQA |
| | PGKGLEWVSGISWNSYSLGYADSVKGRFTISRDNAKNSLYLQMNSLRIEDTALYYCAKEF |
| | IGWDLPYFDYWGQGTLVTVSSGGGGSGGGGSGGGGSGGGGSDIVMTQSPDSLAVSLGERAT |
| | HIDCKSSQNVLYSSSNKNYLAWYQQKPGQPPNLLIYWASTRESGVPDRESGSGSGTDFTLT |
| | ISSLQAEDVAVYYCQQYYSIPYTFGQGTKLEIKAAAIEVMYPPPYLDNEKSNGTHIHVKG |
| | KHLCPSPLFPGPSKPFWVLVVVGGVLACYSLLVTVAFIIFWVRSKRSRLLHSDYMNMTPR |
| | RPGPTRKHYQPYAPPRDFAAYRSRVKFSRSADAPAYQQGQNQLYNELNLGRREEYDVLDK |
| | RRGRDPEMGGKPRRKNPQEGLYNELQKDKMAEAYSEIGMKGERRRGKGHDGLYQGLSTAT |
| | KDTYDALHMQALPPR*** |
| 105 | METDTLLLWVLLLWVPGSTGQVQLQQSGPGLVKPSQTLSLTCAISGDSVSSNSAAWNWIR |
| | QSPSRGLEWLGRTYYRSKWYNDYAVSVKSRITINPDTSKNQFSLQLNSVTPEDTAVYYCA |
| | REVTGDLEDAFDIWGQGTMVTVSSGGGGGGGGSGGGGSGGGGSDIQMTQSPSSLSASVG |
| | DRVTITCRASQTIWSYLNWYQQRPGKAPNLLIYAASSLQSGVPSRESGRGSGTDFTLTIS |
| | SLQAEDFATYYCQQSYSIPQTFGQGTKLEIKASTTTPAPRPPTPAPTIASQPLSLRPEAC |
| | RPAAGGAVHTRGLDFACDIYIWAPLAGTCGVLLLSLVITLYCNHRNVRSKRSRLLHSDYM |
| | NMTPRRPGPTRKHYQPYAPPRDFAAYRSRVKFSRSADAPAYQQGQNQLYNELNIGRREEY |
| | DVLDKRRGRDPEMGGKPRRKNPQEGLYNELQKDKMAEAYSEIGMKGERRRGKGHDGLYQG |
| | LSTATKDTYDALHMQALPPR*** |
| 106 | METDTLLLWVILLWVPGSTGQVQLQQSGPGLVKPSQTLSLTCAISGDSVSSNSAAWNWIR |
| | QSPSRGLEWLGRTYYRSKWYNDYAVSVKSRITINPDTSKNQFSLQLNSVTPEDTAVYYCA |
| | REVTGDLEDAFDIWGQGTMVTVSSGGGGGGGGSGGGGSGGGGSDIQMTQSPSSLSASVG |
| | DRVTITCRASQTIWSYLNWYQQRPGKAPNLLIYAASSLQSGVPSRESGRGSGTDFTLTIS |
| | SLQAEDFATYYCQQSYSIPQTFGQGTKLEIKASLSNSIMYFSHFVPVFLPAKPTTTPAPR |
| | IPPTPAPTIASQPLSLRPEACRPAAGGAVHTRGLDFACDIYIWAPLAGTCGVLLLSLVITL |
| | YCNHRNVRSKRSRLLHSDYMNMTPRRPGPTRKHYQPYAPPRDFAAYRSRVKFSRSADAPA |
| | YQQGQNQLYNELNLGRREEYDVLDKRRGRDPEMGGKPRRKNPQEGLYNELQKDKMAEAYS |
| | EIGMKGERRRGKGHDGLYQGLSTATKDTYDALHMQALPPR*** |
| 107 | METDTLLLWVLLLWVPGSTGQVQLQQSGPGLVKPSQTLSLTCAISGDSVSSNSAAWNWIR |
| | QSPSRGLEWLGRTYYRSKWYNDYAVSVKSRITINPDTSKNQFSLQLNSVTPEDTAVYYCA |
| | REVTGDLEDAFDIWGQGTMVTVSSGGGGSDIQMTQSPSSLSASVGDRVTITCRASQTIWS |
| | YLNWYQQRPGKAPNLLIYAASSLQSGVPSRESGRGSGTDFTLTISSLQAEDFATYYCQQS |
| | YSIPQTFGQGTKLEIKASTTTPAPRPPTPAPTIASQPLSLRPEACRPAAGGAVHTRGLDF |
| | ACDIYIWAPLAGTCGVLLLSLVITLYCNHRNVRSKRSRLLHSDYMNMTPRRPGPTRKHYQ |
| | PYAPPRDFAAYRSRVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLSTATKDTYDALHMQ |
| | ALPPR*** |
| 108 | AUGGCUCUCCCAGUGACCGCCCUGCUGCUGCCCCUAGCCCUCCUUUUGCACGCAGCGAGGCCUCAGGUCCAGCUCGUGGAGAGCGGGGGGGGCGUG |
| | GUGCAGCCCGGCCGAAGCCUCCGCCUAAGCUGCGCCGCCAGCGGGUUCACCUUCAGCAGCUACGGCAUGCACUGGGUACGGCAGGCCCCCGGCAAAG |
| | GACUGGAGUGGGUGGCGAUCAUAUACUACGACGGGAGCAAGAAAUACUACGCCGACUCCGUCAAGGGCAGGUUUACCAUCAGCCGCGACAACAGCA |
| | AGAACACGCUGUACCUGCAGAUGAACAGCCUGAGGGCCGAGGACACCGCCGUCUACUACUGUGCAAGAGAGCUAACCGGCGACGCGUUUGACAUCU |
| | GGGGGCAGGGCACCAAGGUGACCGUGUCCUCCGGGGGCGGGGGCAGCGGCGGCGGCGGCUCCGGCGGCGGGGGGUCAGGGGGGGGCGGCAGCGAG |
| | AUAGUGCUGACCCAGACCCCAUCCAGCCUGUCCGCCUCCCUGGGCGACCGCGUGACGAUCAGCUGCCGGGCCAGCCAGUCGAUCGGCAGCAGCCUAC |
| | ACUGGUACCAGCAGAAGCCGGACCAGUCCCCCAAGCUGCUCAUCAAGUACGCCUCCCAGUCAUUCUCCGGGGUGCCCUCAAAGUUCAUCGGGAGCG |
| | GGUCCGGCACGGACUUCACACUCACCAUCAACAGCCUGGAGGCCGAGGACGCCGCGACCUUUUACUGCCACCAAAGCAGCACCCUCCCCUACACCUUC |
| | GGCCAGGGCACAAAGCUGGAGAUCAAGGCGGCCGCGAUAGAGGUAAUGUACCCUCCUCCUUACCUGGACAACGAGAAGUCGAACGGCACCAUCAUC |
| | CACGUCAAGGGCAAGCACCUCUGCCCCUCGCCCCUGUUCCCCGGCCCCUCCAAGCCCUUCUGGGUGCUGGUCGUGGUUGGUGGCGUCCUCGCCUGC |
| | UACUCCCUCCUCGUCACGGUGGCGUUCAUCAUCUUCUGGGUGCGGAGCAAGCGAAGCCGCCUCCUGCACAGCGACUACAUGAACAUGACGCCCCGC |
| | CGCCCCGGCCCGACCCGGAAGCACUACCAGCCCUACGCCCCGCCGCGCGACUUCGCGGCCUACCGCUCCCGGGUCAAGUUCUCCCGCAGCGCCGACGC |
| | CCCCGCGUACCAGCAGGGCCAGAACCAGCUCUACAACGAGCUCAACCUGGGGAGGCGCGAGGAGUACGACGUGCUUGACAAGCGCCGUGGCCGGGA |
| | CCCUGAGAUGGGGGGUAAGCCGCGACGGAAGAACCCGCAGGAGGGCCUGUACAACGAGCUGCAGAAGGACAAGAUGGCGGAGGCCUACAGCGAGA |
| | UCGGGAUGAAGGGGGAGCGGCGGCGUGGCAAGGGGCACGACGGCCUGUACCAGGGCCUCAGUACGGCCACCAAGGACACCUACGACGCCUUGCACA |
| | UGCAGGCCCUCCCCCCCCGCUAGUGAUGA |
| 109 | AUGGCUCUCCCAGUGACCGCCCUGCUGCUGCCCCUAGCCCUCCUUUUGCACGCAGCGAGGCCUCAGGUGCAGCUGGUGCAGAGCGGCGCCGAGGUG |
| | AAGAAACCCGGCGCGAGCGUCAAAGUGAGCUGCAAAGCAAGCGGCUAUACAUUCACCGGCUACUACAUACACUGGGUGAGGCAAGCCCCAGGCCAG |
| | GGCCUCGAGUGGAUGGGCUGGAUCAAUCCCAACUCCGGCGGCACGAACUAUGCCCAGAAGUUCCAGGGCAGGGUGACCAUGACACGAGACACCAGC |
| | AUCGGCACCGCCUACAUGGAACUGUCAAGGCUGCGAUCCGACGACACCGCCAUUUACUACUGCACCCGGGAGGAUACCUGGAACUACAUCGACUAU |
| | UGGGGCCAGGGGACCCUCGUGACAGUCAGCUCCGGGGGGGGGGGGAGCGGGGGCGGGGGCAGCGGCGGCGGCGGCUCAGGCGGCGGGGGGAGCG |
| | ACAUCCAGAUGACCCAAAGCCCCAGCAGCCUCUCCGCGAGCGUCGGGGACCGAGUGACCAUCACCUGCCGCGCCUCCCAGAGCAUCAGCUCCCAUCUC |
| | AACUGGUACCAGCAGAAGCCCGGCAAGGCCCCCAAGCUACUGAUCUACGCCGCCUCAAGCCUCCACAGCGGCGUACCCAGCCGCUUUAGCGGGUCCG |
| | GCAGCGGCACCGACUUCACCCUCACGAUCUCCUCCCUGCAGCCCGAGGACUUCGCCACCUACUGCUGCCAGCAGAGCUACAGCACCCCCAGGACGUUC |
| | GGCCAGGGCACCAAAGUGGAAAUCAAGGCGGCCGCGAUAGAGGUAAUGUACCCUCCUCCUUACCUGGACAACGAGAAGUCGAACGGCACCAUCAUC |
| | CACGUCAAGGGCAAGCACCUCUGCCCCUCGCCCCUGUUCCCCGGCCCCUCCAAGCCCUUCUGGGUGCUGGUCGUGGUUGGUGGCGUCCUCGCCUGC |
| | UACUCCCUCCUCGUCACGGUGGCGUUCAUCAUCUUCUGGGUGCGGAGCAAGCGAAGCCGCCUCCUGCACAGCGACUACAUGAACAUGACGCCCCGC |
| | CGCCCCGGCCCGACCCGGAAGCACUACCAGCCCUACGCCCCGCCGCGCGACUUCGCGGCCUACCGCUCCCGGGUCAAGUUCUCCCGCAGCGCCGACGC |
| | CCCCGCGUACCAGCAGGGCCAGAACCAGCUCUACAACGAGCUCAACCUGGGGAGGCGCGAGGAGUACGACGUGCUUGACAAGCGCCGUGGCCGGGA |
| | CCCUGAGAUGGGGGGUAAGCCGCGACGGAAGAACCCGCAGGAGGGCCUGUACAACGAGCUGCAGAAGGACAAGAUGGCGGAGGCCUACAGCGAGA |
| | UCGGGAUGAAGGGGGAGCGGCGGCGUGGCAAGGGGCACGACGGCCUGUACCAGGGCCUCAGUACGGCCACCAAGGACACCUACGACGCCUUGCACA |
| | UGCAGGCCCUCCCCCCCCGCUAGUGAUGA |
| 110 | AUGGCUCUCCCAGUGACCGCCCUGCUGCUGCCCCUAGCCCUCCUUUUGCACGCAGCGAGGCCUCAGGUGACCCUGAGGGAGAGGGGGCCCGCCCUG |
| | GUGAAGCCGACACAGACACUGACCCUCACGUGCACCUUCAGCGGCAUCAGCCUGACGACCUCCGGCAUGUGCGUGAGCUGGAUCAGGCAGCCCCCCG |
| | GGAAGGCCCUGGAGUGGCUAGCCCUCAUCGACUGGGACGGGGACAACUACUACUCCACCUCACUCAAGACAAGGCUGACCAUAAGCAAGGACACCA |
| | GCAAGAACCAGGUCGUGCUGACCAUGGCCAGCAUGGAUCCGGUGGAUACCGCCACCUACUAUUGCGCCCGGAUGGGAUACACCUACGGCUACGACG |
| | CCUUUGACAUCUGGGGGCAGGGCACAAUGGUGACGGUCUCGUCCGGAGGGGGGGGCUCCGGAGGGGGAGGCAGCGGCGGCGGCGGAUCGGGGGG |
| | GGGGGGCUCCGAGAUCGUCCUCACCCAAACCCCCUCAAGCCUGAGCGCAAGCCUGGGCGACCGCGUGACCAUCUCCUGCAGGGCCAGCCAAUCAAUC |
| | GGCAGCCUGCUCCACUGGUACCAGCAGAAACCCGACCAGUCCCCAAAGCUGCUGAUCAAGUUCGCCAGCCAGUCACUGAGCGGCGUGCCCAGCCGCU |
| | UCAGCGGCAGCGGCUCCGGCACCGACUUCACCCUGACAAUAAACUCCCUCGAAGCCGAGGACGCCGCCACCUACUACUGCCACCAGAGCAGGAGCCU |
| | GCCCUUCACCUUCGGACCCGGCACCAAGGUGGACAUCAACGCAAGCAGCCUGAGGCCGGAGGCAUGCCGGCCAGCAGCCGGGGGCGCGGUACACACC |
| | CGCGGCCUGGACUUCGCGUGUGACAUCUACAUCUGGGCCCCCCUGGCCGGCACGUGCGGCGUCCUGCUGCUGAGCCUCGUGAUCACACUGUACUGC |
| | AACCACCGGAACGUCAGGUCCAAGAGGAGCCGCCUGCUCCACAGCGACUIACAUGAACAUGACCCCGAGGCGACCCGGCCCCACGAGGAAACACUACCA |
| | GCCCUACGCCCCACCCAGGGACUUCGCGGCCUAUCGAAGCCGGGUGAAGUUCUCACGCAGCGCCGAUGCCCCCGCCUACCAGCAGGGGCAGAACCAG |
| | CUCUACAACGAACUGAACCUGGGACGACGGGAGGAGUACGACGUACUGGAUAAGCGGCGUGGGCGUGACCCCGAAAUGGGGGGGAAGCCCCGGAG |
| | GAAGAACCCCCAGGAGGGACUGUACAACGAGCUGCAGAAGGACAAGAUGGCGGAGGCGUACAGCGAGAUCGGCAUGAAGGGCGAGCGAAGGCGUG |
| | GCAAGGGGCACGACGGCCUGUAUCAGGGGCUGUCAACCGCAACCAAGGACACAUACGACGCCCUCCACAUGCAAGCCCUGCCCCCCCGCUAGUGAUGA |
| 111 | AUGGCUCUCCCAGUGACCGCCCUGCUGCUGCCCCUAGCCCUCCUUUUGCACGCAGCGAGGCCUCAAGUCACCCUGCGGGAGAGCGGCCCCGCCCUGG |
| | UCAAGACCACGCAGACCCUGACCCUCACCUGCACCUUCAGCGGCUUUAGCCUCAGCACCAGCGGCAUGUGCGUGAGCUGGAUCCGCCAGCCCCCCGG |
| | GAAGGCACUGGAGUGGCUCGCCCUGAUCGACUGGGAUGAUGACAAAUACUACAGCACCAGCCUGAAGACCCGCCUGACGAUCUCAAAGGAUACCAG |
| | CAAGAGCCAGGUGGUCCUGACCAUGACCAGCAUGGACCCCGUCGACACCGCCACCUACUACUGCGCCCGCAUCAGGUACAUCUACGGAUACGACUAC |
| | UUCGAUUACUGGGGCCAGGGAACCCUGGUGACCGUGUCGAGCGGGGGGGGAGGGAGCGGCGGGGGGGGCAGCGGCGGCGGGGGGUCCGGCGGGG |
| | GGGGCUCCGACAUCCAGAUGACCCAGAGCCCCAGCAGCCUCUCCGCCAGCGUGGGGGAUCGUGUGACCAUCACCUGUCGCGCGUCCCAACGAAUAA |
| | GCAGCCAGCUCAAUUGGUACCAGCAGAAGCCAGGGAAAGCACCCAAGCUGCUGAUCUACGCAGCCAGCAGCCUGCAGAGCGGCGUCCCCUCACGCUU |
| | CUCAGGCAGCGGCUCCGGAACAGAGUUCACGCUGACAAUCAGCUCCCUGCAGCCAGAGGACAUAGCGACCUAUUGCUGCCAACAGACCUACAGCAAG |
| | CCCAUCACGUUCGGCCAAGGAACCCGGCUGGAGAUCAAGGCGGCCGCGAUAGAGGUAAUGUACCCUCCUCCUUACCUGGACAACGAGAAGUCGAAC |
| | GGCACCAUCAUCCACGUCAAGGGCAAGCACCUCUGCCCCUCGCCCCUGUUCCCCGGCCCCUCCAAGCCCUUCUGGGUGCUGGUCGUGGUUGGUGGC |
| | GUCCUCGCCUGCUACUCCCUCCUCGUCACGGUGGCGUUCAUCAUCUUCUGGGUGCGGAGCAAGCGAAGCCGCCUCCUGCACAGCGACUACAUGAAC |
| | AUGACGCCCCGCCGCCCCGGCCCGACCCGGAAGCACUIACCAGCCCUACGCCCCGCCGCGCGACUUCGCGGCCUACCGCUCCCGGGUCAAGUUCUCCCG |
| | CAGCGCCGACGCCCCCGCGUACCAGCAGGGCCAGAACCAGCUCUACAACGAGCUCAACCUGGGGAGGCGCGAGGAGUACGACGUGCUUGACAAGCGC |
| | CGUGGCCGGGACCCUGAGAUGGGGGGUAAGCCGCGACGGAAGAACCCGCAGGAGGGCCUGUACAACGAGCUGCAGAAGGACAAGAUGGCGGAGGC |
| | CUACAGCGAGAUCGGGAUGAAGGGGGAGCGGCGGCGUGGCAAGGGGCACGACGGCCUGUACCAGGGCCUCAGUACGGCCACCAAGGACACCUACGA |
| | CGCCUUGCACAUGCAGGCCCUCCCCCCCCGCUAGUGAUGA |
| 112 | AUGGCUCUCCCAGUGACCGCCCUGCUGCUGCCCCUAGCCCUCCUUUUGCACGCAGCGAGGCCUGAGGUGCAGCUGGUGGAGAGCGGGGGGGGCCU |
| | GGUGCAGCCCGGCAGGAGCCUGAGGCUGAGCUGCGCCGCCAGCGGCUUCACCUUCGACGGCUACGCCAUGCACUGGGUGAGGCAGGCCCCCGGCAA |
| | GGGCCUGGAGUGGGUGAGCGGCAUCAGCUGGAACAGCUACAGCCUGGGCUACGCCGACAGCGUGAAGGGCAGGUUCACCAUCAGCAGGGACAACG |
| | CCAAGAACAGCCUGUACCUGCAGAUGAACAGCCUGAGGAUCGAGGACACCGCCCUGUACUACUGCGCCAAGGAGUUCGGCUGGGACCUGCCCUACU |
| | UCGACUACUGGGGCCAGGGCACCCUGGUGACCGUGAGCAGGGGGGGGGGGGCAGCGGCGGGGGGGGCAGCGGCGGCGGCGGCAGCGGGGGGGGC |
| | GGCAGCGACAUCGUGAUGACCCAGAGCCCCGACAGCCUGGCCGUGAGCCUGGGCGAGAGGGCCACCAUCGACUGCAAGAGCAGCCAGAACGUGCUG |
| | UACAGCAGCAGCAACAAGAACUACCUGGCCUGGUACCAGCAGAAGCCCGGCCAGCCCCCCAACCUGCUGAUCUACUGGGCCAGCACCAGGGAGAGCG |
| | GCGUGCCCGACAGGUUCAGCGGCAGCGGCAGCGGCACCGACUUCACCCUGACCAUCAGCAGCCUGCAGGCCGAGGACGUGGCCGUGUACUACUGCC |
| | AGCAGUACUACAGCAUCCCCUACACCUUCGGCCAGGGCACCAAGCUGGAGAUCAAGGCGGCCGCGAUAGAGGUAAUGUACCCUCCUCCUUACCUGG |
| | ACAACGAGAAGUCGAACGGCACCAUCAUCCACGUCAAGGGCAAGCACCUCUGCCCCUCGCCCCUGUUCCCCGGCCCCUCCAAGCCCULICUGGGUGCU |
| | GGUCGUGGUUGGUGGCGUCCUCGCCUGCUACUCCCUCCUCGUCACGGUGGCGUUCAUCAUCUUCUGGGUGCGGAGCAAGCGAAGCCGCCUCCUGC |
| | ACAGCGACUACAUGAACAUGACGCCCCGCCGCCCCGGCCCGACCCGGAAGCACUACCAGCCCUACGCCCCGCCGCGCGACUUCGCGGCCUACCGCUCCC |
| | GGGUCAAGUUCUCCCGCAGCGCCGACGCCCCCGCGUACCAGCAGGGCCAGAACCAGCUCUACAACGAGCUCAACCUGGGGAGGCGCGAGGAGUACGA |
| | CGUGCUUGACAAGCGCCGUGGCCGGGACCCUGAGAUGGGGGGUAAGCCGCGACGGAAGAACCCGCAGGAGGGCCUGUACAACGAGCUGCAGAAGG |
| | ACAAGAUGGCGGAGGCCUACAGCGAGAUGGGGAUGAAGGGGGAGCGGCGGCGUGGCAAGGGGCACGACGGCCUGUACCAGGGCCUCAGUACGGCC |
| | ACCAAGGACACCUACGACGCCUUGCACAUGCAGGCCCUCCCCCCCCGCUAGUGAUGA |
| 113 | AUGGCUCUCCCAGUGACCGCCCUGCUGCUGCCCCUAGCCCUCCUUUUGCACGCAGCGAGGCCUCAGGUGCAGCUGCAGCAGUCCGGCCCAGGGCUG |
| | GUGAAGCCAUCCCAGACCCUGAGCCUGACCUGCGCCAUCUCAGGCGACAGCGUCUCCAGCAACUCCGCCGCCUGGAAUUGGAUCCGGCAGAGCCCCA |
| | GCAGGGGCCUCGAGUGGCUGGGGCGUACCUACUACCGCAGCAAGUGGUACAACGACUACGCGGUGAGCGUGAAGUCAAGGAUCACCAUCAACCCCG |
| | AUACAAGCAAGAACCAAUUCUCACUGCAGCUGAACUCCGUGACGCCCGAGGACACAGCCGUGUACUACUGCGCCCGGGAGGUGACCGGCGACCUGG |
| | AAGACGCCUUCGACAUCUGGGGACAGGGCACAAUGGUGACGGUCAGCUCCGGCGGGGGGGGGUCGGACAUCCAGAUGACGCAGAGCCCCAGCAGC |
| | CUGAGCGCCAGCGUAGGAGACCGCGUCACCAUCACCUGCAGGGCAAGCCAGACCAUCUGGAGCUACCUGAACUGGUACCAGCAGCGUCCCGGCAAG |
| | GCCCCCAAUCUGCUGAUCUACGCCGCCUCCAGCCUGCAGUCCGGAGUGCCCUCGAGGUUCAGCGGCCGGGGCAGGGGGACCGACUUCACCCUGACCA |
| | UCAGCUCGCUGCAGGCCGAGGACUUUGCCACCUACUACUGCCAACAGAGCUACUCAAUCCCCCAGACGUUUGGCCAGGGCACCAAGCUGGAGAUCA |
| | AGGCCAGCACCACAACCCCCGCGCCCCGGCCCCCCACCCCCGCCCCCACCAUCGCCAGCCAGCCGCUGAGCCUGAGGCCGGAAGCGUGCCGACCGGCCG |
| | CCGGGGGCGCCGUACACACCCGCGGACUCGACUUCGCCUGCGACAUAUAUAUCUGGGCCCCCCUGGCCGGCACCUGCGGCGUCCUGCUCCUGAGCCU |
| | GGUGAUCACCCUGUACUGCAACCACAGGAACGUCCGGUCCAAGCGUUCCCGCCUCCUGCACAGCGAUUACAUGAACAUGACCCCCCGACGCCCCGGC |
| | CCAACCCGAAAGCACUACCAGCCAUAUGCCCCCCCCAGGGACUUUGCCGCCUAUCGAUCCAGGGUGAAGUUCAGCAGGUCAGCGGACGCGCCCGCCU |
| | ACCAGCAGGGCCAGAACCAGCUGUAUAACGAGCUGAACCUCGGGCGGCGAGAGGAGUACGACGUGCUCGACAAGCGCCGGGGCAGGGACCCCGAAA |
| | UGGGAGGGAAGCCCCGCAGGAAAAACCCCCAGGAGGGACUCUACAACGAGCUCCAAAAGGAUAAGAUGGCCGAAGCCUACAGCGAGAUCGGCAUGA |
| | AAGGGGAGAGGAGGCGGGGCAAGGGGCACGACGGCCUGUACCAGGGACUGUCAACCGCCACCAAGGACACCUACGACGCCCUGCACAUGCAGGCCC |
| | UGCCGCCCAGGUAGUGAUGA |
| 114 | AUGGCUCUCCCAGUGACCGCCCUGCUGCUGCCCCUAGCCCUCCUUUUGCACGCAGCGAGGCCUGAGGUGAAGCUGCAGGAGUCCGGCCCCGGGCUG |
| | GUCGCGCCCAGCCAGAGCCUCAGCGUGACCUGCACCGUGAGCGGCGUGAGCCUGCCCGACUACGGCGUCAGCUGGAUCCGCCAGCCCCCCAGGAAGG |
| | GGCUGGAGUGGCUGGGCGUGAUCUGGGGCAGUGAGACGACCUAUUACAACUCCGCCCUCAAGAGCCGCCUAACCAUCAUCAAGGACAACAGCAAGA |
| | GCCAGGUGUUCCUGAAGAUGAACAGCCUGCAGACCGACGACACCGCCAUCUACUACUGCGCCAAGCACUACUACUACGGGGGUCCUACGCCAUGG |
| | ACUACUGGGGGCAGGGCACCAGCGUGACCGUCAGCUCGGGGGGGGGGGGCAGCGGGGGAGGCGGGUCGGGGGGGGCGGCAGCGACAUCCAGAUG |
| | ACCCAGACCACCAGCUCCCUGAGCGCCUCCCUCGGCGACCGAGUCACGAUCAGCUGCAGGGCCUCCCAGGACAUCAGCAAGUACCUCAACUGGUACC |
| | AGCAGAAGCCGGACGGGACCGUGAAGCUGCUGAUCUACCACACCUCCCGGCUGCACAGCGGCGUGCCCAGCCGGUUCAGCGGCUCCGGCAGCGGCA |
| | CCGACUACAGCCUGACCAUCAGCAACCUGGAGCAGGAGGACAUCGCCACCUACUUCUGCCAGCAGGGGAACACCCUGCCCUIACACGUUCGGCGGGGG |
| | CACCAAGCUGGAGAUCAAGGCGGCCGCGAUAGAGGUAAUGUACCCUCCUCCUUACCUGGACAACGAGAAGUCGAACGGCACCAUCAUCCACGUCAA |
| | GGGCAAGCACCUCUGCCCCUCGCCCCUGUUCCCCGGCCCCUCCAAGCCCUUCUGGGUGCUGGUCGUGGUUGGUGGCGUCCUCGCCUGCUACUCCCU |
| | CCUCGUCACGGUGGCGUUCAUCAUCUUCUGGGUGCGGAGCAAGCGAAGCCGCCUCCUGCACAGCGACUACAUGAACAUGACGCCCCGCCGCCCCGG |
| | CCCGACCCGGAAGCACUACCAGCCCUACGCCCCGCCGCGCGACUUCGCGGCCUACCGCUCCCGGGUCAAGUUCUCCCGCAGCGCCGACGCCCCCGCGU |
| | ACCAGCAGGGCCAGAACCAGCUCUACAACGAGCUCAACCUGGGGAGGCGCGAGGAGUACGACGUGCUUGACAAGCGCCGUGGCCGGGACCCUGAGA |
| | UGGGGGGUAAGCCGCGACGGAAGAACCCGCAGGAGGGCCUGUACAACGAGCUGCAGAAGGACAAGAUGGCGGAGGCCUACAGCGAGAUCGGGAUG |
| | AAGGGGGAGCGGCGGCGUGGCAAGGGGCACGACGGCCUGUACCAGGGCCUCAGUACGGCCACCAAGGACACCUACGACGCCUUGCACAUGCAGGCC |
| | CUCCCCCCCCGCUAGUGAUGA |
| 115 | AUGGCUCUCCCAGUGACCGCCCUGCUGCUGCCCCUAGCCCUCCUUUUGCACGCAGCGAGGCCUCAGGUCCAGCUCGUGGAGAGCGGGGGGGGCGUG |
| | GUGCAGCCCGGCCGAAGCCUCCGCCUAAGCUGCGCCGCCAGCGGGUUCACCUUCAGCAGCUACGGCAUGCACUGGGUACGGCAGGCCCCCGGCAAAG |
| | GACUGGAGUGGGUGGCGAUCAUAUACUACGACGGGAGCAAGAAAUACUACGCCGACUCCGUCAAGGGCAGGUUUACCAUCAGCCGCGACAACAGCA |
| | AGAACACGCUGUACCUGCAGAUGAACAGCCUGAGGGCCGAGGACACCGCCGUCUACUACUGUGCAAGAGAGCUAACCGGCGACGCGUUUGACAUCU |
| | GGGGGCAGGGCACCAAGGUGACCGUGUCCUCCGGGGGGGGGGGGAGCGGGGGGGGCGGCUCCGGCGGGGGGGGUCAGGGGGGGGCGGCAGCGAG |
| | AUAGUGCUGACCCAGACCCCAUCCAGCCUGUCCGCCUCCCUGGGCGACCGCGUGACGAUCAGCUGCCGGGCCAGCCAGUCGAUCGGCAGCAGCCUAC |
| | ACUGGUACCAGCAGAAGCCGGACCAGUCCCCCAAGCUGCUCAUCAAGUACGCCUCCCAGUCAUUCUCCGGGGUGCCCUCAAAGUUCAUCGGGAGCG |
| | GGUCCGGCACGGACUUCACACUCACCAUCAACAGCCUGGAGGCCGAGGACGCCGCGACCUUUUACUGCCACCAAAGCAGCACCCUCCCCUACACCUUC |
| | GGCCAGGGCACAAAGCUGGAGAUCAAGGCGGCCGCGAUAGAGGUAAUGUACCCUCCUCCUUACCUGGACAACGAGAAGUCGAACGGCACCAUCAUC |
| | CACGUCAAGGGCAAGCACCUCUGCCCGUCGCCGCUGUUCCCGGGCCCGUCCAAGCCCUUCUGGGUGCUGGUCGUGGUUGGUGGCGUCCUCGCCUG |
| | CUACUCCCUCCUCGUCACGGUGGCGUUCAUCAUCUUCUGGAAGCGCGGCAGGAAGAAGCUCCUGUACAUCUUCAAGCAGCCGUUCAUGCGCCCGGU |
| | GCAGACGACCCAGGAGGAGGACGGGUGCAGCUGCCGGUUCCCGGAGGAGGAGGAGGGGGGCUGCGAGCUGCGGGUCAAGUUCUCCCGCAGCGCCG |
| | ACGCGCCCGCGUACCAGCAGGGCCAGAACCAGCUCUACAACGAGCUCAACCUGGGGAGGCGCGAGGAGUACGACGUGCUUGACAAGCGCCGUGGCC |
| | GGGACCCUGAGAUGGGCGGUAAGCCGCGACGGAAGAACCCGCAGGAGGGCCUGUACAACGAGCUGCAGAAGGACAAGAUGGCGGAGGCCUACAGC |
| | GAGAUCGGGAUGAAGGGCGAGCGGCGGCGUGGCAAGGGCCACGACGGCCUGUACCAGGGCCUCAGUACGGCCACCAAGGACACCUACGACGCCUUG |
| | CACAUGCAGGCCCUCCCGCCGCGCUAGUGAUGA |
| 116 | MALPVTALLLPLALLLHAARPQVQLVESGGGVVQPGRSLRLSCAASGFTFSSYGMHWVRQ |
| | APGKGLEWVAIIYYDGSKKYYADSVKGRFTISRDNSKNTLYLQMNSLRAEDTAVYYCARE |
| | LTGDAFDIWGQGTKVTVSSGGGGSGGGGSGGGGSGGGGSEIVLTQTPSSLSASLGDRVTI |
| | SCRASQSIGSSLHWYQQKPDQSPKLLIKYASQSFSGVPSKFIGSGSGTDFTLTINSLEAE |
| | DAATFYCHQSSTLPYTFGQGTKLEIKAAAIEVMYPPPYLDNEKSNGTIIHVKGKHLCPSP |
| | LFPGPSKPFWVLVVVGGVLACYSLLVTVAFIIFWVRSKRSRLLHSDYMNMTPRRPGPTRK |
| | HYQPYAPPRDFAAYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLSTATKDTYDAL |
| | HMQALPPR*** |
| 117 | MALPVTALLLPLALLLHAARPQVQLVQSGAEVKKPGASVKVSCKASGYTFTGYYIHWVRQ |
| | APGQGLEWMGWINPNSGGTNYAQKFQGRVTMTRDTSIGTAYMELSRLRSDDTAIYYCTRE |
| | DTWNYIDYWGQGTLVTVSSGGGGSGGGGSGGGGSGGGGSDIQMTQSPSSLSASVGDRVTI |
| | TCRASQSISSHLNWYQQKPGKAPKLLIYAASSLHSGVPSRFSGSGSGTDFTLTISSLQPE |
| | DFATYCCQQSYSTPRTFGQGTKVEIKAAAIEVMYPPPYLDNEKSNGTHIHVKGKHLCPSP |
| | LFPGPSKPFWVLVVVGGVLACYSLLVTVAFIIFWVRSKRSRLLHSDYMNMTPRRPGPTRK |
| | HYQPYAPPRDFAAYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLSTATKDTYDAL |
| | HMQALPPR*** |
| 118 | MALPVTALLLPLALLLHAARPQVTLRESGPALVKPTQTLTLTCTESGISLTTSGMCVSWI |
| | RQPPGKALEWLALIDWDGDNYYSTSLKTRLTISKDTSKNQVVLTMASMDPVDTATYYCAR |
| | MGYTYGYDAFDIWGQGTMVTVSSGGGGSGGGGSGGGGSGGGGSEIVLTQTPSSLSASLGD |
| | RVTISCRASQSIGSLLHWYQQKPDQSPKLLIKFASQSLSGVPSRFSGSGSGTDFTLTINS |
| | LEAEDAATYYCHQSRSLPFTFGPGTKVDINASSLRPEACRPAAGGAVHTRGLDFACDIYI |
| | WAPLAGTCGVLLLSLVITLYCNHRNVRSKRSRLLHSDYMNMTPRRPGPTRKHYQPYAPPR |
| | DFAAYRSRVKFSRSADAPAYQQGQNQLYNELNLGRREEYDVLDKRRGRDPEMGGKPRRKN |
| | PQEGLYNELQKDKMAEAYSEIGMKGERRRGKGHDGLYQGLSTATKDTYDALHMQALPPR*** |
| 119 | MALPVTALLLPLALLLHAARPQVTLRESGPALVKTTQTLTLTCTFSGFSLSTSGMCVSWI |
| | RQPPGKALEWLALIDWDDDKYYSTSLKTRLTISKDTSKSQVVLTMTSMDPVDTATYYCAR |
| | IRYIYGYDYFDYWGQGTLVTVSSGGGGSGGGGSGGGGSGGGGSDIQMTQSPSSLSASVGD |
| | RVTITCRASQRISSQLNWYQQKPGKAPKLLIYAASSLQSGVPSRFSGSGSGTEFTLTISS |
| | LQPEDIATYCCQQTYSKPITFGQGTRLEIKAAAIEVMYPPPYLQNEKSNGTIIHVKGKHL |
| | CPSPLFPGPSKPFWVLVVVGGVLACYSLLVTVAFIIFWVRSKRSRLLHSDYMNMTPRRPG |
| | PTRKHYQPYAPPRDFAAYRSRVKFSRSADAPAYQQGQNQLYNELNLGRREEYDVLDKRRG |
| | RDPEMGGKPRRKNPQEGLYNELQKDKMAEAYSEIGMKGERRRGKGHDGLYQGLSTATKDT |
| | YDALHMQALPPR*** |
| 120 | MALPVTALLLPLALLLHAARPEVQLVESGGGLVQPGRSLRLSCAASGFTFDGYAMHWVRQ |
| | APGKGLEWVSGISWNSYSLGYADSVKGRFTISRDNAKNSLYLQMNSLRIEDTALYYCAKE |
| | FGWDLPYFDYWGQGTLVTVSSGGGGSGGGGSGGGGSGGGGSDIVMTQSPDSLAVSLGERA |
| | TIDCKSSQNVLYSSSNKNYLAWYQQKPGQPPNLLIYWASTRESGVPDRFSGSGSGTDFTL |
| | TISSLQAEDVAVYYCQQYYSIPYTFGQGTKLEIKAAAIEVMYPPPYLDNEKSNGTIIHVK |
| | GKHLCPSPLFPGPSKPFWVLVVVGGVLACYSLLVTVAFIIFWVRSKRSRLLHSDYMNMTP |
| | RRPGPTRKHYQPYAPPRDFAAYRSRVKFSRSADAPAYQQGQNQLYNELNLGRREEYDVLD |
| | KRRGRDPEMGGKPRRKNPQEGLYNELQKDKMAEAYSEIGMKGERRRGKGHDGLYQGLSTA |
| | TKDTYDALHMQALPPR*** |
| 121 | MALPVTALLLPLALLLHAARPQVQLQQSGPGLVKPSQTLSLTCAISGDSVSSNSAAWNW! |
| | IRQSPSRGLEWLGRTYYRSKWYNDYAVSVKSRITINPDTSKNQFSLQLNSVTPEDTAVYYC |
| | AREVTGDLEDAFDIWGQGTMVTVSSGGGGSDIQMTQSPSSLSASVGDRVTITCRASQTIW |
| | SYLNWYQQRPGKAPNLLIYAASSLQSGVPSRFSGRGSGTDFTLTISSLQAEDFATYYCQQ |
| | SYSIPQTFGQGTKLEIKASTTTPAPRPPTPAPTIASQPLSLRPEACRPAAGGAVHTRGLD |
| | FACDIYIWAPLAGTCGVLLLSLVITLYCNHRNVRSKRSRLLHSDYMNMTPRRPGPTRKHY |
| | QPYAPPRDFAAYRSRVKFSRSADAPAYQQGQNQLYNELNLGRREEYDVLDKRRGRDPEMG |
| | GKPRRKNPQEGLYNELQKDKMAEAYSEIGMKGERRRGKGHDGLYQGLSTATKDTYDALHM |
| | QALPPR*** |
| 122 | MALPVTALLLPLALLLHAARPEVKLQESGPGLVAPSQSLSVTCTVSGVSLPDYGVSWIRQ |
| | PPRKGLEWLGVIWGSETTYYNSALKSRLTIIKDNSKSQVFLKMNSLQTDDTAIYYCAKHY |
| | YYGGSYAMDYWGQGTSVTVSSGGGGSGGGGSGGGGSDIQMTQTTSSLSASLGDRVTISCR |
| | ASQDISKYLNWYQQKPDGTVKLLIYHTSRLHSGVPSRFSGSGSGTDYSLTISNLEQEDIA |
| | TYFCQQGNTLPYTFGGGTKLEIKAAAIEVMYPPPYLDNEKSNGTHIHVKGKHLCPSPLFP |
| | GPSKPFWVLVVVGGVLACYSLLVTVAFIIFWVRSKRSRLLHSDYMNMTPRRPGPTRKHYQ |
| | PYAPPRDFAAYRSRVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLSTATKDTYDALHMQ |
| | ALPPR*** |
| 123 | MALPVTALLLPLALLLHAARPQVQLVESGGGVVQPGRSLRLSCAASGFTFSSYGMHWVRQ |
| | APGKGLEWVAIIYYDGSKKYYADSVKGRFTISRDNSKNTLYLQMNSLRAEDTAVYYCARE |
| | LTGDAFDIWYGQGTKVTVSSGGGGSGGGGSGGGGSGGGGSEIVLTQTPSSLSASLGDRVTI |
| | SCRASQSIGSSLHWYQQKPDQSPKLLIKYASQSFSGVPSKFIGSGSGTDFTLTINSLEAE |
| | DAATFYCHQSSTLPYTFGQGTKLEIKAAAIEVMYPPPYLDNEKSNGTIIHVKGKHLCPSP |
| | LFPGPSKPFWVLVVVGGVLACYSLLVTVAFIIFWKRGRKKLLYIFKQPFMRPVQTTQEED |
| | GCSCRFPEEEEGGCELRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLSTATKDTYDAL |
| | HMQALPPR*** |
| 124 | AUGGCCCUGCCCGUGACAGCACUGCUGCUGCCAUUAGCUCUGCUGCUGCACGCCGCUAGACCACAGGUGCAGCUGGUGGAGAGCGGAGGAGGAGU |
| | GGUCCAGCCCGGCAGAUCCCUCAGACUGAGCUGCGCCGCUUCCGGCUIUCACCUUCAGCUCCUACGGCAUGCACUGGGUGAGACAGGCUCCAGGCAA |
| | GGGCCUGGAAUGGGUCGCAAUCAUCUACUACGAUGGGUCUAAGAAGUAUUACGCCGACUCCGUGAAGGGGAGGUUCACAAUCAGCAGAGACAAUA |
| | GCAAGAAUACUCUGUAUCUGCAGAUGAACUCCCUGAGAGCCGAGGACACAGCUGUGUACUACUGCGCCCGCGAGCUGACUGGCGAUGCAUUCGAC |
| | AUCUGGGGGCAGGGGACCAAGGUGACCGUGUCCUCUGGCGGCGGAGGCUCUGGAGGAGGAGGCUCUGGAGGAGGGGGCAGCGGCGGAGGAGGAA |
| | GUGAGAUCGUGCUGACCCAGACCCCUAGCAGCCUGAGCGCCUCCCUGGGCGACAGAGUGACCAUCAGCUGCCGGGCCUCUCAGAGCAUCGGCAGCU |
| | CCCUGCACUGGUACCAGCAGAAGCCUGACCAGAGCCCAAAGCUGCUGAUCAAGUACGCCAGCCAGAGCUUUAGCGGCGUGCCCAGCAAGUUCAUCG |
| | GCAGCGGCUCCGGCACCGAUUUCACCCUGACCAUCAAUUCCCUGGAGGCCGAAGAUGCUGCCACCUUCUACUGCCACCAGAGCAGCACCCUGCCCUA |
| | CACCUUCGGCCAGGGCACCAAGCUGGAGAUCAAAGCCGCCGCCAUCGAAGUGAUGUACCCCCCCCCCUACCUGGACAACGAGAAGUCCAACGGCACC |
| | AUCAUCCACGUGAAAGGCAAGCACCUGUGCCCCUCCCCCCUGUUCCCUGGACCUUCCAAGCCUUUCUGGGUGCUGGUGGUGGUGGGCGGCGUGCU |
| | GGCCUGCUACAGCCUGCUGGUGACAGUGGCCUUCAUCAUCUUCUGGGUGAGAAGCAAGCGGUCCCGGCUGCUGCACAGCGACUACAUGAACAUGA |
| | CCCCCAGAAGACCCGGCCCUIACCAGAAAGCACUACCAGCCCUACGCCCCCCCCAGAGAUUUCGCCGCCUIACAGAAGCCGGGUGAAGUUCAGCAGAAGC |
| | GCCGACGCCCCUGCCUACCAGCAGGGCCAGAAUCAGCUGUACAACGAGCUGAACCUGGGCAGAAGAGAGGAGUACGACGUGCUGGACAAGAGAAGA |
| | GGCAGAGAUCCCGAGAUGGGGGGCAAGCCCAGACGGAAGAACCCUCAGGAGGGCCUGUACAAUGAGCUGCAGAAGGACAAGAUGGCCGAGGCCUAC |
| | AGCGAGAUCGGCAUGAAGGGCGAGAGAAGAAGAGGCAAGGGCCACGACGGCCUGUACCAGGGCCUGAGCACCGCCACAAAGGACACCUACGAUGCC |
| | CUGCACAUGCAGGCCCUGCCCCCUAGAUAGUGAUAA |
| 125 | AUGGCCCUGCCCGUGACAGCACUGCUGCUGCCAUUAGCUCUGCUGCUGCACGCCGCUAGACCACAGGUGCAGCUGGUGGAGAGCGGAGGAGGAGU |
| | GGUCCAGCCCGGCAGAUCCCUCAGACUGAGCUGCGCCGCUUCCGGCUUCACCUUCAGCUCCUACGGCAUGCACUGGGUGAGACAGGCUCCAGGCAA |
| | GGGCCUGGAAUGGGUCGCAAUCAUCUACUACGAUGGGUCUAAGAAGUAUUACGCCGACUCCGUGAAGGGGAGGUUCACAAUCAGCAGAGACAAUA |
| | GCAAGAAUACUCUGUAUCUGCAGAUGAACUCCCUGAGAGCCGAGGACACAGCUGUGUACUACUGCGCCCGCGAGCUGACUGGCGAUGCAUUCGAC |
| | AUCUGGGGGCAGGGGACCAAGGUGACCGUGUCCUCUGGCGGCGGAGGCUCUGGAGGAGGAGGCUCUGGAGGAGGGGGCAGCGGCGGAGGAGGAA |
| | GUGAGAUCGUGCUGACCCAGACCCCUAGCAGCCUGAGCGCCUCCCUGGGCGACAGAGUGACCAUCAGCUGCCGGGCCUCUCAGAGCAUCGGCAGCU |
| | CCCUGCACUGGUACCAGCAGAAGCCUGACCAGAGCCCAAAGCUGCUGAUCAAGUACGCCAGCCAGAGCUUUAGCGGCGUGCCCAGCAAGUUCAUCG |
| | GCAGCGGCUCCGGCACCGAUUUCACCCUGACCAUCAAUUCCCUGGAGGCCGAAGAUGCUGCCACCUUCUACUGCCACCAGAGCAGCACCCUGCCCUA |
| | CACCUUCGGCCAGGGCACCAAGCUGGAGAUCAAAGCCGCCGCCAUCGAAGUGAUGUACCCCCCCCCCUACCUGGACAACGAGAAGUCCAACGGCACC |
| | AUCAUCCACGUGAAAGGCAAGCACCUGUGCCCCUCCCCCCUGULCCCUGGACCUUCCAAGCCUUUCUGGGUGCUGGUGGUGGUGGGCGGCGUGCU |
| | GGCCUGCUACAGCCUGCUGGUGACAGUGGCCUUCAUCAUCUUCUGGAAGCGGGGCAGAAAGAAGCUGCUGUACAUCUUCAAGCAGCCCUUCAUGC |
| | GGCCCGUGCAGACCACCCAGGAGGAGGAUGGCUGCAGCUGCAGAUUUCCUGAGGAGGAGGAGGGGGGCUGCGAGCUGCGGGUGAAGUUCAGCAG |
| | AAGCGCCGACGCCCCUGCCUACCAGCAGGGCCAGAAUCAGCUGUACAACGAGCUGAACCUGGGCAGAAGAGAGGAGUACGACGUGCUGGACAAGAG |
| | AAGAGGCAGAGAUCCCGAGAUGGGGGGCAAGCCCAGACGGAAGAACCCUCAGGAGGGCCUGUACAAUGAGCUGCAGAAGGACAAGAUGGCCGAGGC |
| | CUACAGCGAGAUCGGCAUGAAGGGCGAGAGAAGAAGAGGCAAGGGCCACGACGGCCUGUACCAGGGCCUGAGCACCGCCACAAAGGACACCUACGA |
| | UGCCCUGCACAUGCAGGCCCUGCCCCCUAGAUAGUGAUAA |
| 126 | MALPVTALLLPLALLLHAARPQVQLVESGGGVVQPGRSLRLSCAASGFTFSSYGMHWVRQ |
| | APGKGLEWVAHIYYDGSKKYYADSVKGRFTISRDNSKNTLYLQMNSLRAEDTAVYYCARE |
| | LTGDAFDIWGQGTKVTVSSGGGGSGGGGSGGGGSGGGGSEIVLTQTPSSLSASLGDRVTI |
| | SCRASQSIGSSLHWYQQKPDQSPKLLIKYASQSFSGVPSKFIGSGSGTDFTLTINSLEAE |
| | DAATFYCHQSSTLPYTFGQGTKLEIKAAAIEVMYPPPYLDNEKSNGTIIHVKGKHLCPSP |
| | LFPGPSKPFWVLVVVGGVLACYSLLVTVAFIIFWVRSKRSRLLHSDYMNMTPRRPGPTRK |
| | HYQPYAPPRDFAAYRSRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLSTATKDTYDAL |
| | HMQALPPR*** |
| 127 | MALPVTALLLPLALLLHAARPQVQLVESGGGVVQPGRSLRLSCAASGFTESSYGMHWVRQ |
| | APGKGLEWVAHIYYDGSKKYYADSVKGRFTISRDNSKNTLYLQMNSLRAEDTAVYYCARE |
| | LTGDAFDIWGQGTKVTVSSGGGGSGGGGSGGGGSGGGGSEIVLTQTPSSLSASLGDRVTI |
| | SCRASQSIGSSLHWYQQKPDQSPKLLIKYASQSFSGVPSKFIGSGSGTDFTLTINSLEAE |
| | DAATFYCHQSSTLPYTFGQGTKLEIKAAAIEVMYPPPYLDNEKSNGTIIHVKGKHLCPSP |
| | LFPGPSKPFWVLVVVGGVLACYSLLVTVAFIIFWKRGRKKLLYIFKQPFMRPVQTTQEED |
| | GCSCRFPEEEEGGCELRVKFSRSADAPAYQQGQNQLYNELNLGRREEYDVLDKRRGRDPE |
| | MGGKPRRKNPQEGLYNELQKDKMAEAYSEIGMKGERRRGKGHDGLYQGLSTATKDTYDAL |
| | HMQALPPR*** |
| 128 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUCCAGCUCGUGGAGAGCGGCGGCGGCGUGGUGCAGCCCGGCCGAAGCCUCCGCCUAAGCUGCGCCGCCAGCGGGUUCACC |
| | UUCAGCAGCUACGGCAUGCACUGGGUACGGCAGGUUUUUGGCAAAGGACUGGAGUGGGUGGCGAUCAUAUACUACGACGGGAGCAAGAAAUACUA |
| | CGCCGACUCCGUCAAGGGCAGGUUUACCAUCAGCCGCGACAACAGCAAGAACACGCUGUACCUGCAGAUGAACAGCCUGAGGGCCGAGGACACCGCC |
| | GUCUACUACUGUGCAAGAGAGCUAACCGGCGACGCGUUUGACAUCUGGGGGCAGGGCACCAAGGUGACCGUGUCCUCCGGGGGCGGGGGCAGCGG |
| | CGGCGGCGGCUCCGGCGGGGGGGGUCAGGCGGCGGCGGCAGCGAGAUAGUGCUGACCCAGACCCCAUCCAGCCUGUCCGCCUCCCUGGGCGACCG |
| | CGUGACGAUCAGCUGCCGGGCCAGCCAGUCGAUCGGCAGCAGCCUACACUGGUACCAGCAGAAGCCGGACCAGUCCCCCAAGCUGCUCAUCAAGUAC |
| | GCCUCCCAGUCAUUCUCCGGGGUGCCCUCAAAGUUCAUCGGGAGCGGGUCCGGCACGGACUUCACACUCACCAUCAACAGCCUGGAGGCCGAGGAC |
| | GCCGCGACCUUUUACUGCCACCAAAGCAGCACCCUCCCCUACACCUUCGGCCAGGGCACAAAGCUGGAGAUCAAGGCGGCCGCGAUAGAGGUAAUG |
| | UACCCUCCUCCUUACCUGGACAACGAGAAGUCGAACGGCACCAUCAUCCACGUCAAGGGCAAGCACCUCUGCCCCUCGCCCCUGUUCCCQGGCCCCUC |
| | CAAGCCCUUCUGGGUGCUGGUCGUGGUUGGUGGCGUCCUCGCCUGCUACUCCCUCCUCGUCACGGUGGCGUUCAUCAUCUUCUGGGUGCGGAGC |
| | AAGCGAAGCCGCCUCCUGCACAGCGACUACAUGAACAUGACGCCCCGCCGCCCCGGCCCGACCCGGAAGCACUACCAGCCCUACGCCCCGCCGCGCGAC |
| | UUCGCGGCCUACCGCUCCCGGGUCAAGUUCUCCCGCAGCGCCGACGCCCCCGCGUACCAGCAGGGCCAGAACCAGCUCUACAACGAGCUCAACCUGG |
| | GGAGGCGCGAGGAGUACGACGUGCUUGACAAGCGCCGUGGCCGGGACCCUGAGAUGGGGGGUAAGCCGCGACGGAAGAACCCGCAGGAGGGCCUG |
| | UACAACGAGCUGCAGAAGGACAAGAUGGCGGAGGCCUACAGCGAGAUCGGGAUGAAGGGGGAGCGGCGGCGUGGCAAGGGGCACGACGGCCUGUA |
| | CCAGGGCCUCAGUACGGCCACCAAGGACACCUACGACGCCUUGCACAUGCAGGCCCUCCCCCCCCGCUAGUGAUGAGCGGCCGCUUAAUUAAGCUGC |
| | CUUCUGCGGGGCUUGCCUUCUGGCCAUGCCCUUCUUCUCUCCCUUGCACCUGUACCUCUUGGUCUUUGAAUAAAGCCUGAGUAGGAAG |
| 129 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGCAGCUGGUGCAGAGCGGCGCCGAGGUGAAGAAACCCGGCGCGAGCGUCAAAGUGAGCUGCAAAGCAAGCGGCUAUAC |
| | AUUCACCGGCUACUACAUACACUGGGUGAGGCAAGCCCCAGGCCAGGGCCUCGAGUGGAUGGGCUGGAUCAAUCCCAACUCCGGCGGCACGAACUA |
| | UGCCCAGAAGUUCCAGGGCAGGGUGACCAUGACACGAGACACCAGCAUCGGCACCGCCUACAUGGAACUGUCAAGGCUGCGAUCCGACGACACCGCC |
| | AUUUACUACUGCACCCGGGAGGAUACCUGGAACUACAUCGACUAUUGGGGCCAGGGGACCCUCGUGACAGUCAGCUCCGGGGGGGGGGGGAGCGG |
| | CGGCGGGGGCAGCGGCGGCGGCGGCUCAGGGGGGGGGGGGAGCGACAUCCAGAUGACCCAAAGCCCCAGCAGCCUCUCCGCGAGCGUCGGGGACCG |
| | AGUGACCAUCACCUGCCGCGCCUCCCAGAGCAUCAGCUCCCAUCUCAACUGGUACCAGCAGAAGCCCGGCAAGGCCCCCAAGCUACUGAUCUACGCC |
| | GCCUCAAGCCUCCACAGCGGCGUACCCAGCCGCUUUAGCGGGUCCGGCAGCGGCACCGACUUCACCCUCACGAUCUCCUCCCUGCAGCCCGAGGACU |
| | UCGCCACCUACUGCUGCCAGCAGAGCUACAGCACCCCCAGGACGUUCGGCCAGGGCACCAAAGUGGAAAUCAAGGCGGCCGCGAUAGAGGUAAUGU |
| | ACCCUCCUCCUUACCUGGACAACGAGAAGUCGAACGGCACCAUCAUCCACGUCAAGGGCAAGCACCUCUGCCCCUCGCCCCUGUUCCCCGGCCCCUCC |
| | AAGCCCUUCUGGGUGCUGGUCGUGGUUGGUGGCGUCCUCGCCUGCUACUCCCUCCUCGUCACGGUGGCGUUCAUCAUCUUCUGGGUGCGGAGCA |
| | AGCGAAGCCGCCUCCUGCACAGCGACUACAUGAACAUGACGCCCCGCCGCCCCGGCCCGACCCGGAAGCACUACCAGCCCUACGCCCCGCCGCGCGAC |
| | UUCGCGGCCUACCGCUCCCGGGUCAAGUUCUCCCGCAGCGCCGACGCCCCCGCGUACCAGCAGGGCCAGAACCAGCUCUACAACGAGCUCAACCUGG |
| | GGAGGCGCGAGGAGUACGACGUGCUUGACAAGCGCCGUGGCCGGGACCCUGAGAUGGGGGGUAAGCCGCGACGGAAGAACCCGCAGGAGGGCCUG |
| | UACAACGAGCUGCAGAAGGACAAGAUGGCGGAGGCCUACAGCGAGAUCGGGAUGAAGGGGGAGCGGCGGCGUGGCAAGGGGCACGACGGCCUGUA |
| | CCAGGGCCUCAGUACGGCCACCAAGGACACCUACGACGCCUUGCACAUGCAGGCCCUCCCCCCCCGCUAGUGAUGAGGGGCCGCUUAAUUAAGCUGC |
| 130 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGCAGCUGGUGCAGAGCGGCGCCGAGGUGAAGAAACCCGGCGCGAGCGUCAAAGUGAGCUGCAAAGCAAGCGGCUAUAC |
| | AUUCACCGGCUACUACAUACACUGGGUGAGGCAAGCCCCAGGCCAGGGCCUCGAGUGGAUGGGCUGGAUCAAUCCCAACUCCGGCGGCACGAACUA |
| | UGCCCAGAAGUUCCAGGGCAGGGUGACCAUGACACGAGACACCAGCAUCGGCACCGCCUACAUGGAACUGUCAAGGCUGCGAUCCGACGACACCGCC |
| | AUUUACUACUGCACCCGGGAGGAUACCUGGAACUACAUCGACUAUUGGGGCCAGGGGACCCUCGUGACAGUCAGCUCCGGGGGGGGGGGGAGCGG |
| | CGGCGGGGGCAGCGGCGGGGGGGGCUCAGGCGGCGGGGGGAGCGACAUCCAGAUGACCCAAAGCCCCAGCAGCCUCUCCGCGAGCGUCGGGGACCG |
| | AGUGACCAUCACCUGCCGCGCCUCCCAGAGCAUCAGCUCCCAUCUCAACUGGUACCAGCAGAAGCCCGGCAAGGCCCCCAAGCUACUGAUCUACGCC |
| | GCCUCAAGCCUCCACAGCGGCGUACCCAGCCGCUUUAGCGGGUCCGGCAGCGGCACCGACUUCACCCUCACGAUCUCCUCCCUGCAGCCCGAGGACU |
| | UCGCCACCUACUGCUGCCAGCAGAGCUACAGCACCCCCAGGACGUUCGGCCAGGGCACCAAAGUGGAAAUCAAGGCGAGCACCACCACCCCCGCGCCC |
| | CGGCCGCCGACCCCCGCCCCCACAAUCGCCUCACAGCCACUGAGCCUGCGCCCCGAGGCCUGCAGGCCCGCCGCAGGCGGCGCCGUGCACACGCGUGG |
| | GCUCGACUUCGCCUGUGACAUAUACAUCUGGGCCCCCCUGGCCGGCACCUGCGGGGUCCUGCUGCUGAGCCUGGUGAUCACCCUGUAUUGCAACCA |
| | CCGGAACGUGCGCUCCAAACGCAGCCGGCUGCUGCACAGCGACUACAUGAACAUGACCCCGCGCAGGCCCGGCCCGACCCGAAAGCACUACCAGCCAU |
| | ACGCCCCCCCCCGGGACUUCGCCGCCUACCGGUCAAGGGUUAAGUUCUCACGGUCGGCCGACGCCCCCGCCLACCAGCAGGGCCAGAACCAGCUGUA |
| | CAAUGAGCUGAACCUGGGCCGGAGGGAGGAGUACGACGUACUGGAUAAAAGGCGCGGCAGAGACCCCGAGAUGGGGGGGAAACCCAGGCGUAAGA |
| | ACCCCCAGGAGGGCCUGUACAACGAACUGCAAAAGGACAAGAUGGCCGAGGCCUACAGCGAAAUAGGCAUGAAGGGCGAGCGUAGGAGGGGGAAG |
| | GGCCACGACGGCCUGUAUCAAGGCCUGUCGACCGCCACCAAAGACACCUACGACGCCCUGCACAUGCAGGCCCUGCCCCCCAGGUAGUGAUGAGCGG |
| | CCGCUUAAUUAAGCUGCCUUCUGGGGGCUUGCCUUCUGGCCAUGCCCUUCUUCUCUCCCUUGCACCUGUACCUCUUGGUCUUUGAAUAAAGCCU |
| | GAGUAGGAAG |
| 131 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGACCCUGAGGGAGAGCGGGCCCGCCCUGGUGAAGCCGACACAGACACUGACCCUCACGUGCACCUUCAGCGGCAUCAGC |
| | CUGACGACCUCCGGCAUGUGCGUGAGCUGGAUCAGGCAGCCCCCCGGGAAGGCCCUGGAGUGGCUAGCCCUCAUCGACUGGGACGGGGACAACUIAC |
| | UACUCCACCUCACUCAAGACAAGGCUGACCAUAAGCAAGGACACCAGCAAGAACCAGGUCGUGCUGACCAUGGCCAGCAUGGAUCCGGUGGAUACC |
| | GCCACCUACUAUUGCGCCCGGAUGGGAUACACCUACGGCUACGACGCCUUUGACAUCUGGGGGCAGGGCACAAUGGUGACGGUCUCGUCCGGAGG |
| | CGGCGGCUCCGGAGGGGGAGGCAGCGGCGGCGGCGGAUCGGGGGGGGGGGGCUCCGAGAUCGUCCUCACCCAAACCCCCUCAAGCCUGAGCGCAAG |
| | CCUGGGCGACCGCGUGACCAUCUCCUGCAGGGCCAGCCAAUCAAUCGGCAGCCUGCUCCACUGGUACCAGCAGAAACCCGACCAGUCCCCAAAGCUG |
| | CUGAUCAAGUUCGCCAGCCAGUCACUGAGCGGCGUGCCCAGCCGCUUCAGCGGCAGCGGCUCCGGCACCGACUUCACCCUGACAAUAAACUCCCUCG |
| | AAGCCGAGGACGCCGCCACCUACUACUGCCACCAGAGCAGGAGCCUGCCCUUCACCUUCGGACCCGGCACCAAGGUGGACAUCAACGCAAGCAGCCU |
| | GAGGCCGGAGGCAUGCCGGCCAGCAGCCGGGGGCGCGGUACACACCCGCGGCCUGGACUUCGCGUGUGACAUCUACAUCUGGGCCCCCCUGGCCGG |
| | CACGUGCGGCGUCCUGCUGCUGAGCCUCGUGAUCACACUGUACUGCAACCACCGGAACGUCAGGUCCAAGAGGAGCCGCCUGCUCCACAGCGACUA |
| | CAUGAACAUGACCCCGAGGCGACCCGGCCCCACGAGGAAACACUACCAGCCCUACGCCCCACCCAGGGACUUCGCGGCCUAUCGAAGCCGGGUGAAG |
| | UUCUCACGCAGCGCCGAUGCCCCCGCCUACCAGCAGGGGCAGAACCAGCUCUACAACGAACUGAACCUGGGACGACGGGAGGAGUACGACGUACUG |
| | GAUAAGCGGCGUGGGCGUGACCCCGAAAUGGGGGGGAAGCCCCGGAGGAAGAACCCCCAGGAGGGACUGUACAACGAGCUGCAGAAGGACAAGAU |
| | GGCGGAGGCGUACAGCGAGAUCGGCAUGAAGGGCGAGCGAAGGCGUGGCAAGGGGCACGACGGCCUGUAUCAGGGGCUGUCAACCGCAACCAAGG |
| | ACACAUACGACGCCCUCCACAUGCAAGCCCUGCCCCCCCGCUAGUGAUGAGCGGCCGCUUAAUUAAGCUGCCUUCUGGGGGGCUUGCCUUCUGGCC |
| | AUGCCCUUCUUCUCUCCCUUGCACCUGUACCUCUUGGUCUUUGAAUAAAGCCUGAGUAGGAAG |
| 132 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGACCCUGAGGGAGAGCGGGCCCGCCCUGGUGAAGCCGACACAGACACUGACCCUCACGUGCACCUUCAGUGGCAUCAGC |
| | CUGACGACCUCCGGCAUGUGCGUGAGCUGGAUCAGGCAGCCCCCCGGGAAGGCCCUGGAGUGGCUAGCCCUCAUCGACUGGGACGGGGACAACUAC |
| | UACUCCACCUCACUCAAGACAAGGCUGACCAUAAGCAAGGACACCAGCAAGAACCAGGUCGUGCUGACCAUGGCCAGCAUGGAUCCGGUGGAUACC |
| | GCCACCUACUAUUGCGCCCGGAUGGGAUACACCUIACGGCUACGACGCCUUUGACAUCUGGGGGCAGGGCACAAUGGUGACGGUCUCGUCCGGAGG |
| | CGGCGGCUCCGGAGGGGGAGGCAGCGGCGGCGGCGGAUCGGGGGGGGGGGGCUCCGAGAUCGUCCUCACCCAAACCCCCUCAAGCCUGAGCGCAAG |
| | CCUGGGCGACCGCGUGACCAUCUCCUGCAGGGCCAGCCAAUCAAUCGGCAGCCUGCUCCACUGGUACCAGCAGAAACCCGACCAGUCCCCAAAGCUG |
| | CUGAUCAAGUUCGCCAGCCAGUCACUGAGCGGCGUGCCCAGCCGCUUCAGCGGCAGCGGCUCCGGCACCGACUUCACCCUGACAAUAAACUCCCUCG |
| | AAGCCGAGGACGCCGCCACCUACUACUGCCACCAGAGCAGGAGCCUGCCCUUCACCUUCGGACCCGGCACCAAGGUGGACAUCAACGCAAGCACCACC |
| | ACCCCCGCCCCCCGCCCCCCCACACCCGCCCCCACCAUCGCAAGCCAGCCCCUGAGCCUGAGGCCGGAGGCAUGCCGGCCAGCAGCCGGGGGCGCGGUA |
| | CACACCCGCGGCCUGGACUUCGCGUGUGACAUCUACAUCUGGGCCCCCCUGGCCGGCACGUGCGGCGUCCUGCUGCUGAGCCUCGUGAUCACACUG |
| | UACUGCAACCACCGGAACGUCAGGUCCAAGAGGAGCCGCCUGCUCCACAGCGACUACAUGAACAUGACCCCGAGGCGACCCGGCCCCACGAGGAAAC |
| | ACUACCAGCCCUACGCCCCACCCAGGGACUUCGCGGCCUAUCGAAGCCGGGUGAAGUUCUCACGCAGCGCCGAUGCCCCCGCCUACCAGCAGGGGCA |
| | GAACCAGCUCUACAACGAACUGAACCUGGGACGACGGGAGGAGUACGACGUACUGGAUAAGCGGCGUGGGCGUGACCCCGAAAUGGGGGGGAAGC |
| | CCCGGAGGAAGAACCCCCAGGAGGGACUGUACAACGAGCUGCAGAAGGACAAGAUGGCGGAGGCGUACAGCGAGAUCGGCAUGAAGGGCGAGCGA |
| | AGGCGUGGCAAGGGGCACGACGGCCUGUAUCAGGGGCUGUCAACCGCAACCAAGGACACAUACGACGCCCUCCACAUGCAAGCCCUGCCCCCCCGCU |
| | AGUGAUGAGCGGCCGCUUAAUUAAGCUGCCUUCUGCGGGGCUUGCCUUCUGGCCAUGCCCUUCUUCUCUCCCUUGCACCUGUACCUCUUGGUCUU |
| | UGAAUAAAGCCUGAGUAGGAAG |
| 133 | AGGAAAUAAGAGAGAAAAGAAGAGUAAGAAGAAAUAUAAGAGCCACCAUGGCUCUCCCAGUGACCGCCCUGCUGCUGCCCCUAGCCCUCCUUUUGC |
| | ACGCAGCGAGGCCUCAGGUGACCCUGAGGGAGAGCGGGCCCGCCCUGGUGAAGCCGACACAGACACUGACCCUCACGUGCACCUUCAGCGGCAUCA |
| | GCCUGACGACCUCCGGCAUGUGCGUGAGCUGGAUCAGGCAGCCCCCCGGGAAGGCCCUGGAGUGGCUAGCCCUCAUCGACUGGGACGGGGACAACU |
| | ACUACUCCACCUCACUCAAGACAAGGCUGACCAUAAGCAAGGACACCAGCAAGAACCAGGUCGUGCUGACCAUGGCCAGCAUGGAUCCGGUGGAUA |
| | CCGCCACCUACUAUUGCGCCCGGAUGGGAUACACCUACGGCUACGACGCCUUUGACAUCUGGGGGCAGGGCACAAUGGUGACGGUCUCGUCCGGA |
| | GGCGGCGGCUCCGGAGGGGGAGGCAGCGGCGGCGGCGGAUCGGGGGGGGGGGGCUCCGAGAUCGUCCUCACCCAAACCCCCUCAAGCCUGAGCGCA |
| | AGCCUGGGCGACCGCGUGACCAUCUCCUGCAGGGCCAGCCAAUCAAUCGGCAGCCUGCUCCACUGGUACCAGCAGAAACCCGACCAGUCCCCAAAGC |
| | UGCUGAUCAAGUUCGCCAGCCAGUCACUGAGCGGCGUGCCCAGCCGCUUCAGCGGCAGCGGCUCCGGCACCGACUUCACCCUGACAAUAAACUCCCU |
| | CGAAGCCGAGGACGCCGCCACCUACUACUGCCACCAGAGCAGGAGCCUGCCCUUCACCUUCGGACCCGGCACCAAGGUGGACAUCAACGCAAGCACCA |
| | CCACCCCCGCCCCCCGCCCCCCCACACCCGCCCCCACCAUCGCAAGCCAGCCCCUGAGCCUGAGGCCGGAGGCAUGCCGGCCAGCAGCCGGGGGCGCGG |
| | UACACACCCGCGGCCUGGACUUCGCGUGUGACAUCUACAUCUGGGCCCCCCUGGCCGGCACGUGCGGCGUCCUGCUGCUGAGCCUCGUGAUCACAC |
| | UGUACUGCAACCACCGGAACGUCAGGUCCAAGAGGAGCCGCCUGCUCCACAGCGACUACAUGAACAUGACCCCGAGGCGACCCGGCCCCACGAGGAA |
| | ACACUACCAGCCCUACGCCCCACCCAGGGACUUCGCGGCCUAUCGAAGCCGGGUGAAGUUCUCACGCAGCGCCGAUGCCCCCGCCUACCAGCAGGGG |
| | CAGAACCAGCUCUACAACGAACUGAACCUGGGACGACGGGAGGAGUACGACGUACUGGAUAAGCGGCGUGGGCGUGACCCCGAAAUGGGGGGGAA |
| | GCCCCGGAGGAAGAACCCCCAGGAGGGACUGUACAACGAGCUGCAGAAGGACAAGAUGGCGGAGGCGUACAGCGAGAUCGGCAUGAAGGGCGAGC |
| | GAAGGCGUGGCAAGGGGCACGACGGCCUGUAUCAGGGGCUGUCAACCGCAACCAAGGACACAUIACGACGCCCUCCACAUGCAAGCCCUGCCCCCCCG |
| | CUAGUGAUGAGCGGCCGCUUAAUUAAGCUGCCUUCUGCGGGGCUUGCCUUCUGGCCAUGCCCUUCUUCUCUCCCUUGCACCUGUACCUCUUGGUC |
| | UUUGAAUAAAGCCUGAGUAGGAAG |
| 134 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAAGUCACCCUGCGGGAGAGCGGCCCCGCCCUGGUCAAGACCACGCAGACCCUGACCCUCACCUGCACCUUCAGQGGCUUUAGCC |
| | UCAGCACCAGCGGCAUGUGCGUGAGCUGGAUCCGCCAGCCCCCCGGGAAGGCACUGGAGUGGCUCGCCCUGAUCGACUGGGAUGAUGACAAAUAC |
| | UACAGCACCAGCCUGAAGACCCGCCUGACGAUCUCAAAGGAUACCAGCAAGAGCCAGGUGGUCCUGACCAUGACCAGCAUGGACCCCGUCGACACCG |
| | CCACCUACUACUGCGCCCGCAUCAGGUACAUCUACGGAUACGACUACUUCGAUUACUGGGGCCAGGGAACCCUGGUGACCGUGUCGAGCGGGGGG |
| | GGAGGGAGCGGCGGCGGGGGCAGCGGCGGCGGCGGGUCCGGCGGGGGGGGCUCCGACAUCCAGAUGACCCAGAGCCCCAGCAGCCUCUCCGCCAGC |
| | GUGGGGGAUCGUGUGACCAUCACCUGUCGCGCGUCCCAACGAAUAAGCAGCCAGCUCAAUUGGUACCAGCAGAAGCCAGGGAAAGCACCCAAGCUG |
| | CUGAUCUACGCAGCCAGCAGCCUGCAGAGCGGCGUCCCCUCACGCUUCUCAGGCAGCGGCUCCGGAACAGAGUUCACGCUGACAAUCAGCUCCCUGC |
| | AGCCAGAGGACAUAGCGACCUAUUGCUGCCAACAGACCUACAGCAAGCCCAUCACGUUCGGCCAAGGAACCCGGCUGGAGAUCAAGGCGGCCGCGA |
| | UAGAGGUAAUGUACCCUCCUCCUUACCUGGACAACGAGAAGUCGAACGGCACCAUCAUCCACGUCAAGGGCAAGCACCUCUGCCCCUCGCCCCUGUU |
| | CCCCGGCCCCUCCAAGCCCUUCUGGGUGCUGGUCGUGGUUGGUGGCGUCCUCGCCUGCUACUCCCUCCUCGUCACGGUGGCGUUCAUCAUCUUCU |
| | GGGUGCGGAGCAAGCGAAGCCGCCUCCUGCACAGCGACUACAUGAACAUGACGCCCCGCCGCCCCGGCCCGACCCGGAAGCACUACCAGCCCUACGCC |
| | CCGCCGCGCGACUUCGCGGCCUACCGCUCCCGGGUCAAGUUCUCCCGCAGCGCCGACGCCCCCGCGUACCAGCAGGGCCAGAACCAGCUCUACAACGA |
| | GCUCAACCUGGGGAGGCGCGAGGAGUACGACGUGCUUGACAAGCGCCGUGGCCGGGACCCUGAGAUGGGGGGUAAGCCGCGACGGAAGAACCCGC |
| | AGGAGGGCCUGUACAACGAGCUGCAGAAGGACAAGAUGGCGGAGGCCUACAGCGAGAUCGGGAUGAAGGGGGAGCGGCGGCGUGGCAAGGGGCA |
| | CGACGGCCUGUACCAGGGCCUCAGUACGGCCACCAAGGACACCUACGACGCCUUGCACAUGCAGGCCCUCCCCCCCCGCUAGUGAUGAGCGGCCGCU |
| | UAAUUAAGCUGCCUUCUGCGGGGCUUGCCUUCUGGCCAUGCCCUUCUUCUCUCCCUUGCACCUGUACCUCUUGGUCUUUGAAUAAAGCCUGAGUA |
| | GGAAG |
| 135 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGGAGGUGCAGCUGGUGGAGAGCGGCGGCGGCCUGGUGCAGCCCGGCAGGAGCCUGAGGCUGAGCUGCGCCGCCAGCGGCUUCAC |
| | CUUCGACGGCUACGCCAUGCACUGGGUGAGGCAGGCCCCCGGCAAGGGCCUGGAGUGGGUGAGCGGCAUCAGCUGGAACAGCUACAGCCUGGGCU |
| | ACGCCGACAGCGUGAAGGGCAGGUUCACCAUCAGCAGGGACAACGCCAAGAACAGCCUGUACCUGCAGAUGAACAGCCUGAGGAUCGAGGACACCG |
| | CCCUGUACUACUGCGCCAAGGAGUUCGGCUGGGACCUGCCCUACUUCGACUACUGGGGCCAGGGCACCCUGGUGACCGUGAGCAGCGGCGGCGGC |
| | GGCAGCGGCGGCGGCGGCAGCGGCGGCGGCGGCAGCGGCGGGGGGGGCAGCGACAUCGUGAUGACCCAGAGCCCCGACAGCCUGGCCGUGAGCCUG |
| | GGCGAGAGGGCCACCAUCGACUGCAAGAGCAGCCAGAACGUGCUGUACAGCAGCAGCAACAAGAACUACCUGGCCUGGUACCAGCAGAAGCCCGGCC |
| | AGCCCCCCAACCUGCUGAUCUACUGGGCCAGCACCAGGGAGAGCGGCGUGCCCGACAGGUUCAGCGGCAGCGGCAGCGGCACCGACUUCACCCUGAC |
| | CAUCAGCAGCCUGCAGGCCGAGGACGUGGCCGUGUACUACUGCCAGCAGUACUACAGCAUCCCCUACACCUUCGGCCAGGGCACCAAGCUGGAGAU |
| | CAAGGCGGCCGCGAUAGAGGUAAUGUACCCUCCUCCUUACCUGGACAACGAGAAGUCGAACGGCACCAUCAUCCACGUCAAGGGCAAGCACCUCUG |
| | CCCCUCGCCCCUGUUCCCCGGCCCCUCCAAGCCCUUCUGGGUGCUGGUCGUGGUUGGUGGCGUCCUCGCCUGCUACUCCCUCCUCGUCACGGUGGC |
| | GUUCAUCAUCUUCUGGGUGCGGAGCAAGCGAAGCCGCCUCCUGCACAGCGACUACAUGAACAUGACGCCCCGCCGCCCCGGCCCGACCCGGAAGCAC |
| | UACCAGCCCUACGCCCCGCCGCGCGACUUCGCGGCCUACCGCUCCCGGGUCAAGUUCUCCCGCAGCGCCGACGCCCCCGCGUACCAGCAGGGCCAGAA |
| | CCAGCUCUACAACGAGCUCAACCUGGGGAGGCGCGAGGAGUACGACGUGCUUGACAAGCGCCGUGGCCGGGACCCUGAGAUGGGGGGUAAGCCGC |
| | GACGGAAGAACCCGCAGGAGGGCCUGUACAACGAGCUGCAGAAGGACAAGAUGGCGGAGGCCUACAGCGAGAUCGGGAUGAAGGGGGAGCGGCGG |
| | CGUGGCAAGGGGCACGACGGCCUGUACCAGGGCCUCAGUACGGCCACCAAGGACACCUACGACGCCUUGCACAUGCAGGCCCUCCCCCCCCGCUAGU |
| | GAUGAGCGGCCGCUUAAUUAAGCUGCCUUCUGGGGGGCUUGCCUUCUGGCCAUGCCCUUCUUCUCUCCCUUGCACCUGUACCUCUUGGUCUUUGA |
| | AUAAAGCCUGAGUAGGAAG |
| 136 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGGAGGUGCAGCUGGUGGAGAGCGGCGGCGGCCUGGUGCAGCCCGGCAGGAGCCUGAGGCUGAGCUGCGCCGCCAGCGGCUUCAC |
| | CUUCGACGGCUACGCCAUGCACUGGGUGAGGCAGGCCCCCGGCAAGGGCCUGGAGUGGGUGAGCGGCAUCAGCUGGAACAGCUACAGCCUGGGCU |
| | ACGCCGACAGCGUGAAGGGCAGGUUCACCAUCAGCAGGGACAACGCCAAGAACAGCCUGUACCUGCAGAUGAACAGCCUGAGGAUCGAGGACACCG |
| | CCCUGUACUACUGCGCCAAGGAGUUCGGCUGGGACCUGCCCUACUUCGACUACUGGGGCCAGGGCACCCUGGUGACCGUGAGCAGCGGGGGGGGC |
| | GGCAGCGGCGGCGGCGGCAGCGGCGGCGGCGGCAGCGGCGGGGGGGGCAGCGACAUCGUGAUGACCCAGAGCCCCGACAGCCUGGCCGUGAGCCUG |
| | GGCGAGAGGGCCACCAUCGACUGCAAGAGCAGCCAGAACGUGCUGUACAGCAGCAGCAACAAGAACUACCUGGCCUGGUACCAGCAGAAGCCCGGCC |
| | AGCCCCCCAACCUGCUGAUCUACUGGGCCAGCACCAGGGAGAGCGGCGUGCCCGACAGGUUCAGCGGCAGCGGCAGCGGCACCGACUUCACCCUGAC |
| | CAUCAGCAGCCUGCAGGCCGAGGACGUGGCCGUGUACUACUGCCAGCAGUACUACAGCAUCCCCUACACCUUCGGCCAGGGCACCAAGCUGGAGAU |
| | CAAGGCGGCCGCGAUAGAGGUAAUGUACCCUCCUCCUUACCUGGACAACGAGAAGUCGAACGGCACCAUCAUCCACGUCAAGGGCAAGCACCUCUG |
| | CCCCUCGCCCCUGUUCCCCGGCCCCUCCAAGCCCUUCUGGGUGCUGGUCGUGGUUGGUGGCGUCCUCGCCUGCUACUCCCUCCUCGUCACGGUGGC |
| | GUUCAUCAUCUUCUGGGUGCGGAGCAAGCGAAGCCGCCUCCUGCACAGCGACUACAUGAACAUGACGCCCCGCCGCCCCGGCCCGACCCGGAAGCAC |
| | UACCAGCCCUACGCCCCGCCGCGCGACUUCGCGGCCUACCGCUCCCGGGUCAAGUUCUCCCGCAGCGCCGACGCCCCCGCGUACCAGCAGGGCCAGAA |
| | CCAGCUCUACAACGAGCUCAACCUGGGGAGGCGCGAGGAGUACGACGUGCUUGACAAGCGCCGUGGCCGGGACCCUGAGAUGGGGGGUAAGCCGC |
| | GACGGAAGAACCCGCAGGAGGGCCUGUACAACGAGCUGCAGAAGGACAAGAUGGCGGAGGCCUACAGCGAGAUCGGGAUGAAGGGGGAGCGGGGG |
| | CGUGGCAAGGGGCACGACGGCCUGUACCAGGGCCUCAGUACGGCCACCAAGGACACCUACGACGCCUUGCACAUGCAGGCCCUCCCCCCCCGCUAGU |
| | GAUGAGCGGCCGCUUAAUUAAGCUGCCUUCUGGGGGGCUUGCCUUCUGGCCAUGCCCUUCUUCUCUCCCUUGCACCUGUACCUCUUGGUCUUUGA |
| | AUAAAGCCUGAGUAGGAAG |
| 137 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGCAGCUGCAGCAGUCCGGCCCAGGGCUGGUGAAGCCAUCCCAGACCCUGAGCCUGACCUGCGCCAUCUCAGGCGACAGC |
| | GUCUCCAGCAACUCCGCCGCCUGGAAUUGGAUCCGGCAGAGCCCCAGCAGGGGCCUCGAGUGGCUGGGGCGUACCUACUACCGCAGCAAGUGGUAC |
| | AACGACUACGCGGUGAGCGUGAAGUCAAGGAUCACCAUCAACCCCGAUACAAGCAAGAACCAAUUCUCACUGCAGCUGAACUCCGUGACGCCCGAG |
| | GACACAGCCGUGUACUACUGCGCCCGGGAGGUGACCGGCGACCUGGAAGACGCCUUCGACAUCUGGGGACAGGGCACAAUGGUGACGGUCAGCUCC |
| | GGCGGGGGGGGGUCGGGGGGGGGGGGGAGCGGAGGCGGCGGCAGCGGCGGCGGCGGAAGCGACAUCCAGAUGACGCAGAGCCCCAGCAGCCUGAG |
| | CGCCAGCGUAGGAGACCGCGUCACCAUCACCUGCAGGGCAAGCCAGACCAUCUGGAGCUACCUGAACUGGUACCAGCAGCGUCCCGGCAAGGCCCCC |
| | AAUCUGCUGAUCUACGCCGCCUCCAGCCUGCAGUCCGGAGUGCCCUCGAGGUUCAGCGGCCGGGGCAGCGGGACCGACUUCACCCUGACCAUCAGC |
| | UCGCUGCAGGCCGAGGACUUUGCCACCUACUACUGCCAACAGAGCUACUCAAUCCCCCAGACGUUUGGCCAGGGCACCAAGCUGGAGAUCAAGGCC |
| | AGCCUUAGCAACAGCAUCAUGUACUUCAGCCACUUUGUCCCCGUCUUCCUGCCAGCCAAGCCGACCACAACCCCCGCGCCCCGGCCCCCCACCCCCGCC |
| | CCCACCAUCGCCAGCCAGCCGCUGAGCCUGAGGCCGGAAGCGUGCCGACCGGCCGCCGGGGGCGCCGUACACACCCGCGGACUCGACUUCGCCUGCG |
| | ACAUAUAUAUCUGGGCCCCCCUGGCCGGCACCUGCGGCGUCCUGCUCCUGAGCCUGGUGAUCACCCUGUACUGCAACCACAGGAACGUCCGGUCCA |
| | AGCGUUCCCGCCUCCUGCACAGCGAUUACAUGAACAUGACCCCCCGACGCCCCGGCCCAACCCGAAAGCACUACCAGCCAUAUGCCCCCCCCAGGGAC |
| | UUUGCCGCCUAUCGAUCCAGGGUGAAGUUCAGCAGGUCAGCGGACGCGCCCGCCUACCAGCAGGGCCAGAACCAGCUGUAUAACGAGCUGAACCUC |
| | GGGCGGCGAGAGGAGUACGACGUGCUCGACAAGCGCCGGGGCAGGGACCCCGAAAUGGGAGGGAAGCCCCGCAGGAAAAACCCCCAGGAGGGACUC |
| | UACAACGAGCUCCAAAAGGAUAAGAUGGCCGAAGCCUACAGCGAGAUCGGCAUGAAAGGGGAGAGGAGGGGGGCAAGGGGCACGACGGCCUGUA |
| | CCAGGGACUGUCAACCGCCACCAAGGACACCUACGACGCCCUGCACAUGCAGGCCCUGCCGCCCAGGUAGUGAUGAGCGGCCGCUUAAUUAAGCUGC |
| | CUUCUGCGGGGCUUGCCUUCUGGCCAUGCCCUUCUUCUCUCCCUUGCACCUGUACCUCUUGGUCUUUGAAUAAAGCCUGAGUAGGAAG |
| 138 | AGGAAAUAAGAGAGAAAAGAAGAGUAAGAAGAAAUAUAAGAGCCACCAUGGAGACCGACACCCUCCUGCUGUGGGUGCUGCUGCUUUGGGUGCCC |
| | GGGAGCACGGGGCAGGUGCAGCUGCAGCAGUCCGGCCCAGGGCUGGUGAAGCCAUCCCAGACCCUGAGCCUGACCUGCGCCAUCUCAGGCGACAGC |
| | GUCUCCAGCAACUCCGCCGCCUGGAAUUGGAUCCGGCAGAGCCCCAGCAGGGGCCUCGAGUGGCUGGGGCGUACCUACUACCGCAGCAAGUGGUAC |
| | AACGACUACGCGGUGAGCGUGAAGUCAAGGAUCACCAUCAACCCCGAUACAAGCAAGAACCAAUUCUCACUGCAGCUGAACUCCGUGACGCCCGAG |
| | GACACAGCCGUGUACUACUGCGCCCGGGAGGUGACCGGCGACCUGGAAGACGCCUUCGACAUCUGGGGACAGGGCACAAUGGUGACGGUCAGCUCC |
| | GGCGGGGGGGGGUCGGACAUCCAGAUGACGCAGAGCCCCAGCAGCCUGAGCGCCAGCGUAGGAGACCGCGUCACCAUCACCUGCAGGGCAAGCCAG |
| | ACCAUCUGGAGCUACCUGAACUGGUACCAGCAGCGUCCCGGCAAGGCCCCCAAUCUGCUGAUCUACGCCGCCUCCAGCCUGCAGUCCGGAGUGCCCU |
| | CGAGGUUCAGCGGCCGGGGCAGCGGGACCGACUUCACCCUGACCAUCAGCUCGCUGCAGGCCGAGGACUUUGCCACCUACUACUGCCAACAGAGCU |
| | ACUCAAUCCCCCAGACGUUUGGCCAGGGCACCAAGCUGGAGAUCAAGGCCAGCACCACAACCCCCGCGCCCCGGCCCCCCACCCCCGCCCCCACCAUCG |
| | CCAGCCAGCCGCUGAGCCUGAGGCCGGAAGCGUGCCGACCGGCCGCCGGGGGCGCCGUIACACACCCGCGGACUCGACUUCGCCUGCGACAUAUAUAU |
| | CUGGGCCCCCCUGGCCGGCACCUGCGGCGUCCUGCUCCUGAGCCUGGUGAUCACCCUGUACUGCAACCACAGGAACGUCCGGUCCAAGCGUUCCCGC |
| | CUCCUGCACAGCGAUUACAUGAACAUGACCCCCCGACGCCCCGGCCCAACCCGAAAGCACUACCAGCCAUAUGCCCCCCCCAGGGACUUUGCCGCCUA |
| | UCGAUCCAGGGUGAAGUUCAGCAGGUCAGCGGACGCGCCCGCCUACCAGCAGGGCCAGAACCAGCUGUAUAACGAGCUGAACCUCGGGCGGCGAGA |
| | GGAGUACGACGUGCUCGACAAGCGCCGGGGCAGGGACCCCGAAAUGGGAGGGAAGCCCCGCAGGAAAAACCCCCAGGAGGGACUCUACAACGAGCU |
| | CCAAAAGGAUAAGAUGGCCGAAGCCUACAGCGAGAUCGGCAUGAAAGGGGAGAGGAGGCGGGGCAAGGGGCACGACGGCCUGUACCAGGGACUGU |
| | CAACCGCCACCAAGGACACCUACGACGCCCUGCACAUGCAGGCCCUGCCGCCCAGGUAGUGAUGAGCGGCCGCUUAAUUAAGCUGCCUUCUGCGGG |
| | GCUUGCCUUCUGGCCAUGCCCUUCUUCUCUCCCUUGCACCUGUACCUCUUGGUCUUUGAAUAAAGCCUGAGUAGGAAG |
| 139 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUDUU |
| | GCACGCAGCGAGGCCUCAGGUCCAGCUCGUGGAGAGCGGCGGCGGCGUGGUGCAGCCCGGCCGAAGCCUCCGCCUAAGCUGCGCCGCCAGCGGGUU |
| | CACCUUCAGCAGCUACGGCAUGCACUGGGUACGGCAGGCCCCCGGCAAAGGACUGGAGUGGGUGGCGAUCAUAUACUACGACGGGAGCAAGAAAU |
| | ACUACGCCGACUCCGUCAAGGGCAGGUUUACCAUCAGCCGCGACAACAGCAAGAACACGCUGUACCUGCAGAUGAACAGCCUGAGGGCCGAGGACAC |
| | CGCCGUCUACUACUGUGCAAGAGAGCUAACCGGCGACGCGUUUGACAUCUGGGGGCAGGGCACCAAGGUGACCGUGUCCUCCGGGGGCGGGGGCA |
| | GCGGGGGGGGCGGCUCCGGCGGCGGCGGGUCAGGCGGCGGCGGCAGCGAGAUAGUGCUGACCCAGACCCCAUCCAGCCUGUCCGCCUCCCUGGGCG |
| | ACCGCGUGACGAUCAGCUGCCGGGCCAGCCAGUCGAUCGGCAGCAGCCUACACUGGUACCAGCAGAAGCCGGACCAGUCCCCCAAGCUGCUCAUCAA |
| | GUACGCCUCCCAGUCAUUCUCCGGGGUGCCCUCAAAGUUCAUCGGGAGCGGGUCCGGCACGGACUUCACACUCACCAUCAACAGCCUGGAGGCCGA |
| | GGACGCCGCGACCUUUUACUGCCACCAAAGCAGCACCCUCCCCUACACCUUCGGCCAGGGCACAAAGCUGGAGAUCAAGGCGGCCGCGAUAGAGGUA |
| | AUGUACCCUCCUCCUUACCUGGACAACGAGAAGUCGAACGGCACCAUCAUCCACGUCAAGGGCAAGCACCUCUGCCCCUCGCCCCUGUUCCCCGGCC |
| | CCUCCAAGCCCUUCUGGGUGCUGGUCGUGGUUGGUGGCGUCCUCGCCUGCUACUCCCUCCUCGUCACGGUGGCGUUCAUCAUCUUCUGGGUGCGG |
| | AGCAAGCGAAGCCGCCUCCUGCACAGCGACUACAUGAACAUGACGCCCCGCCGCCCCGGCCCGACCCGGAAGCACUACCAGCCCUACGCCCCGCCGCGC |
| | GACUUCGCGGCCUACCGCUCCCGGGUCAAGUUCUCCCGCAGCGCCGACGCCCCCGCGUACCAGCAGGGCCAGAACCAGCUCUACAACGAGCUCAACC |
| | UGGGGAGGCGCGAGGAGUACGACGUGCUUGACAAGCGCCGUGGCCGGGACCCUGAGAUGGGGGGUAAGCCGCGACGGAAGAACCCGCAGGAGGGC |
| | CUGUACAACGAGCUGCAGAAGGACAAGAUGGCGGAGGCCUACAGCGAGAUCGGGAUGAAGGGGGAGCGGCGGCGUGGCAAGGGGCACGACGGCCU |
| | GUACCAGGGCCUCAGUACGGCCACCAAGGACACCUACGACGCCUUGCACAUGCAGGCCCUCCCCCCCCGCUAGUGAUGAGGCACAUUUGAAUAAAUU |
| | CUAUUACCAGUUCAAGCUUGGCGUAAUCAUGGUCAUAGCUG |
| 140 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUCAGGUGCAGCUGGUGCAGAGCGGCGCCGAGGUGAAGAAACCCGGCGCGAGCGUCAAAGUGAGCUGCAAAGCAAGCGGCU |
| | AUACAUUCACCGGCUACUACAUACACUGGGUGAGGCAAGCCCCAGGCCAGGGCCUCGAGUGGAUGGGCUGGAUCAAUCCCAACUCCGGCGGCACGA |
| | ACUAUGCCCAGAAGUUCCAGGGCAGGGUGACCAUGACACGAGACACCAGCAUCGGCACCGCCUACAUGGAACUGUCAAGGCUGCGAUCCGACGACA |
| | CCGCCAUUUACUACUGCACCCGGGAGGAUACCUGGAACUACAUCGACUAUUGGGGCCAGGGGACCCUCGUGACAGUCAGCUCCGGGGGGGGGGGG |
| | AGCGGCGGCGGGGGCAGCGGCGGCGGCGGCUCAGGCGGCGGGGGGAGCGACAUCCAGAUGACCCAAAGCCCCAGCAGCCUCUCCGCGAGCGUCGGG |
| | GACCGAGUGACCAUCACCUGCCGCGCCUCCCAGAGCAUCAGCUCCCAUCUCAACUGGUACCAGCAGAAGCCCGGCAAGGCCCCCAAGCUACUGAUCU |
| | ACGCCGCCUCAAGCCUCCACAGCGGCGUACCCAGCCGCUUUAGCGGGUCCGGCAGCGGCACCGACUUCACCCUCACGAUCUCCUCCCUGCAGCCCGAG |
| | GACUUCGCCACCUACUGCUGCCAGCAGAGCUACAGCACCCCCAGGACGUUCGGCCAGGGCACCAAAGUGGAAAUCAAGGCGGCCGCGAUAGAGGUA |
| | AUGUACCCUCCUCCUUACCUGGACAACGAGAAGUCGAACGGCACCAUCAUCCACGUCAAGGGCAAGCACCUCUGCCCCUCGCCCCUGUUCCCCGGCC |
| | CCUCCAAGCCCUUCUGGGUGCUGGUCGUGGUUGGUGGCGUCCUCGCCUGCUACUCCCUCCUCGUCACGGUGGCGUUCAUCAUCUUCUGGGUGCGG |
| | AGCAAGCGAAGCCGCCUCCUGCACAGCGACUACAUGAACAUGACGCCCCGCCGCCCCGGCCCGACCCGGAAGCACUACCAGCCCUACGCCCCGCCGCGC |
| | GACUUCGCGGCCUACCGCUCCCGGGUCAAGUUCUCCCGCAGCGCCGACGCCCCCGCGUACCAGCAGGGCCAGAACCAGCUCUACAACGAGCUCAACC |
| | UGGGGAGGCGCGAGGAGUACGACGUGCUUGACAAGCGCCGUGGCCGGGACCCUGAGAUGGGGGGUAAGCCGCGACGGAAGAACCCGCAGGAGGGC |
| | CUGUACAACGAGCUGCAGAAGGACAAGAUGGCGGAGGCCUACAGCGAGAUCGGGAUGAAGGGGGAGCGGGGGCGUGGCAAGGGGCACGACGGCCU |
| | GUACCAGGGCCUCAGUACGGCCACCAAGGACACCUACGACGCCUUGCACAUGCAGGCCCUCCCCCCCCGCUAGUGAUGAGGCACAUUUGAAUAAAUU |
| | CUAUUACCAGUUCAAGCUUGGCGUAAUCAUGGUCAUAGCUG |
| 141 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUCAGGUGACCCUGAGGGAGAGCGGGCCCGCCCUGGUGAAGCCGACACAGACACUGACCCUCACGUGCACCUUCAGCGGCAU |
| | CAGCCUGACGACCUCCGGCAUGUGCGUGAGCUGGAUCAGGCAGCCCCCCGGGAAGGCCCUGGAGUGGCUAGCCCUCAUCGACUGGGACGGGGACAA |
| | CUACUACUCCACCUCACUCAAGACAAGGCUGACCAUAAGCAAGGACACCAGCAAGAACCAGGUCGUGCUGACCAUGGCCAGCAUGGAUCCGGUGGA |
| | UACCGCCACCUACUAUUGCGCCCGGAUGGGAUACACCUACGGCUACGACGCCUUUGACAUCUGGGGGCAGGGCACAAUGGUGACGGUCUCGUCCG |
| | GAGGCGGCGGCUCCGGAGGGGGAGGCAGCGGCGGCGGCGGAUCGGGGGGGGGGGGCUCCGAGAUCGUCCUCACCCAAACCCCCUCAAGCCUGAGC |
| | GCAAGCCUGGGCGACCGCGUGACCAUCUCCUGCAGGGCCAGCCAAUCAAUCGGCAGCCUGCUCCACUGGUACCAGCAGAAACCCGACCAGUCCCCAA |
| | AGCUGCUGAUCAAGUUCGCCAGCCAGUCACUGAGGGGCGUGCCCAGCCGCUUCAGCGGCAGCGGCUCCGGCACCGACUUCACCCUGACAAUAAACUC |
| | CCUCGAAGCCGAGGACGCCGCCACCUACUACUGCCACCAGAGCAGGAGCCUGCCCUUCACCUUCGGACCCGGCACCAAGGUGGACAUCAACGCAAGC |
| | AGCCUGAGGCCGGAGGCAUGCCGGCCAGCAGCCGGGGGCGCGGUACACACCCGCGGCCUGGACUUCGCGUGUGACAUCUACAUCUGGGCCCCCCUG |
| | GCCGGCACGUGCGGCGUCCUGCUGCUGAGCCUCGUGAUCACACUGUACUGCAACCACCGGAACGUCAGGUCCAAGAGGAGCCGCCUGCUCCACAGC |
| | GACUACAUGAACAUGACCCCGAGGCGACCCGGCCCCACGAGGAAACACUACCAGCCCUACGCCCCACCCAGGGACUUCGCGGCCUAUCGAAGCCGGG |
| | UGAAGUUCUCACGCAGCGCCGAUGCCCCCGCCUACCAGCAGGGGCAGAACCAGCUCUACAACGAACUGAACCUGGGACGACGGGAGGAGUACGACG |
| | UACUGGAUAAGCGGCGUGGGCGUGACCCCGAAAUGGGGGGGAAGCCCCGGAGGAAGAACCCCCAGGAGGGACUGUACAACGAGCUGCAGAAGGAC |
| | AAGAUGGCGGAGGCGUACAGCGAGAUCGGCAUGAAGGGCGAGCGAAGGCGUGGCAAGGGGCACGACGGCCUGUAUCAGGGGCUGUCAACCGCAAC |
| | CAAGGACACAUACGACGCCCUCCACAUGCAAGCCCUGCCCCCCCGCUAGUGAUGAGGCACAUUUGAAUAAAUUCUAUUACCAGUUCAAGCUUGGCG |
| | UAAUCAUGGUCAUAGCUG |
| 142 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUCAAGUCACCCUGCGGGAGAGCGGCCCCGCCCUGGUCAAGACCACGCAGACCCUGACCCUCACCUGCACCUUCAGCGGCUUU |
| | AGCCUCAGCACCAGCGGCAUGUGCGUGAGCUGGAUCCGCCAGCCCCCCGGGAAGGCACUGGAGUGGCUCGCCCUGAUCGACUGGGAUGAUGACAAA |
| | UACUACAGCACCAGCCUGAAGACCCGCCUGACGAUCUCAAAGGAUACCAGCAAGAGCCAGGUGGUCCUGACCAUGACCAGCAUGGACCCCGUCGACA |
| | CCGCCACCUACUACUGCGCCCGCAUCAGGUACAUCUACGGAUACGACUACUUCGAUUACUGGGGCCAGGGAACCCUGGUGACCGUGUCGAGCGGGG |
| | GGGGAGGGAGCGGCGGCGGGGGCAGCGGCGGCGGGGGGUCCGGCGGGGGGGGCUCCGACAUCCAGAUGACCCAGAGCCCCAGCAGCCUCUCCGCCA |
| | GCGUGGGGGAUCGUGUGACCAUCACCUGUCGCGCGUCCCAACGAAUAAGCAGCCAGCUCAAUUGGUACCAGCAGAAGCCAGGGAAAGCACCCAAGC |
| | UGCUGAUCUACGCAGCCAGCAGCCUGCAGAGCGGCGUCCCCUCACGCUUCUCAGGCAGCGGCUCCGGAACAGAGUUCACGCUGACAAUCAGCUCCC |
| | UGCAGCCAGAGGACAUAGCGACCUAUUGCUGCCAACAGACCUACAGCAAGCCCAUCACGUUCGGCCAAGGAACCCGGCUGGAGAUCAAGGCGGCCGC |
| | GAUAGAGGUAAUGUACCCUCCUCCUUACCUGGACAACGAGAAGUCGAACGGCACCAUCAUCCACGUCAAGGGCAAGCACCUCUGCCCCUCGCCCCUG |
| | UUCCCCGGCCCCUCCAAGCCCUUCUGGGUGCUGGUCGUGGUUGGUGGCGUCCUCGCCUGCUACUCCCUCCUCGUCACGGUGGCGUUCAUCAUCUU |
| | CUGGGUGCGGAGCAAGCGAAGCCGCCUCCUGCACAGCGACUACAUGAACAUGACGCCCCGCCGCCCCGGCCCGACCCGGAAGCACUACCAGCCCUACG |
| | CCCCGCCGCGCGACUUCGCGGCCUACCGCUCCCGGGUCAAGUUCUCCCGCAGCGCCGACGCCCCCGCGUACCAGCAGGGCCAGAACCAGCUCUACAAC |
| | GAGCUCAACCUGGGGAGGCGCGAGGAGUACGACGUGCUUGACAAGCGCCGUGGCCGGGACCCUGAGAUGGGGGGUAAGCCGCGACGGAAGAACCC |
| | GCAGGAGGGCCUGUACAACGAGCUGCAGAAGGACAAGAUGGCGGAGGCCUACAGCGAGAUCGGGAUGAAGGGGGAGCGGCGGCGUGGCAAGGGG |
| | CACGACGGCCUGUACCAGGGCCUCAGUACGGCCACCAAGGACACCUACGACGCCUUGCACAUGCAGGCCCUCCCCCCCCGCUAGUGAUGAGGCACAU |
| | UUGAAUAAAUUCUAUUACCAGUUCAAGCUUGGCGUAAUCAUGGUCAUAGCG |
| 143 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUGAGGUGCAGCUGGUGGAGAGCGGCGGCGGCCUGGUGCAGCCCGGCAGGAGCCUGAGGCUGAGCUGCGCCGCCAGCGGCU |
| | UCACCUUCGACGGCUACGCCAUGCACUGGGUGAGGCAGGCCCCCGGCAAGGGCCUGGAGUGGGUGAGCGGCAUCAGCUGGAACAGCUACAGCCUG |
| | GGCUACGCCGACAGCGUGAAGGGCAGGUUCACCAUCAGCAGGGACAACGCCAAGAACAGCCUGUACCUGCAGAUGAACAGCCUGAGGAUCGAGGAC |
| | ACCGCCCUGUACUACUGCGCCAAGGAGUUCGGCUGGGACCUGCCCUACUUCGACUACUGGGGCCAGGGCACCCUGGUGACCGUGAGCAGCGGCGGC |
| | GGCGGCAGCGGCGGCGGCGGCAGCGGCGGCGGCGGCAGCGGCGGCGGCGGCAGCGACAUCGUGAUGACCCAGAGCCCCGACAGCCUGGCCGUGAGC |
| | CUGGGCGAGAGGGCCACCAUCGACUGCAAGAGCAGCCAGAACGUGCUGUACAGCAGCAGCAACAAGAACUACCUGGCCUGGUACCAGCAGAAGCCC |
| | GGCCAGCCCCCCAACCUGCUGAUCUACUGGGCCAGCACCAGGGAGAGCGGCGUGCCCGACAGGUUCAGCGGCAGCGGCAGCGGCACCGACUUCACCC |
| | UGACCAUCAGCAGCCUGCAGGCCGAGGACGUGGCCGUGUACUACUGCCAGCAGUACUACAGCAUCCCCUACACCUUCGGCCAGGGCACCAAGCUGG |
| | AGAUCAAGGCGGCCGCGAUAGAGGUAAUGUACCCUCCUCCUUACCUGGACAACGAGAAGUCGAACGGCACCAUCAUCCACGUCAAGGGCAAGCACC |
| | UCUGCCCCUCGCCCCUGUUCCCCGGCCCCUCCAAGCCCUUCUGGGUGCUGGUCGUGGUUGGUGGCGUCCUCGCCUGCUACUCCCUCCUCGUCACGG |
| | UGGCGUUCAUCAUCUUCUGGGUGCGGAGCAAGCGAAGCCGCCUCCUGCACAGCGACUACAUGAACAUGACGCCCCGCCGCCCCGGCCCGACCCGGAA |
| | GCACUACCAGCCCUACGCCCCGCCGCGCGACUUCGCGGCCUIACCGCUCCCGGGUCAAGUUCUCCCGCAGCGCCGACGCCCCCGCGUACCAGCAGGGCC |
| | AGAACCAGCUCUACAACGAGCUCAACCUGGGGAGGCGCGAGGAGUACGACGUGCUUGACAAGCGCCGUGGCCGGGACCCUGAGAUGGGGGGUAAG |
| | CCGCGACGGAAGAACCCGCAGGAGGGCCUGUACAACGAGCUGCAGAAGGACAAGAUGGCGGAGGCCUACAGCGAGAUCGGGAUGAAGGGGGAGCG |
| | GCGGCGUGGCAAGGGGCACGACGGCCUGUACCAGGGCCUCAGUACGGCCACCAAGGACACCUACGACGCCUUGCACAUGCAGGCCCUCCCCCCCCGC |
| | UAGUGAUGAGGCACAUUUGAAUAAAUUCUAUUACCAGUUCAAGCUUGGCGUAAUCAUGGUCAUAGCUG |
| 144 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUCAGGUGCAGCUGCAGCAGUCCGGCCCAGGGCUGGUGAAGCCAUCCCAGACCCUGAGCCUGACCUGCGCCAUCUCAGGCGA |
| | CAGCGUCUCCAGCAACUCCGCCGCCUGGAAUUGGAUCCGGCAGAGCCCCAGCAGGGGCCUCGAGUGGCUGGGGCGUACCUACUACCGCAGCAAGUG |
| | GUACAACGACUACGCGGUGAGCGUGAAGUCAAGGAUCACCAUCAACCCCGAUACAAGCAAGAACCAAUUCUCACUGCAGCUGAACUCCGUGACGCCC |
| | GAGGACACAGCCGUGUACUACUGCGCCCGGGAGGUGACCGGCGACCUGGAAGACGCCUUCGACAUCUGGGGACAGGGCACAAUGGUGACGGUCAG |
| | CUCCGGCGGGGGGGGGUCGGACAUCCAGAUGACGCAGAGCCCCAGCAGCCUGAGCGCCAGCGUAGGAGACCGCGUCACCAUCACCUGCAGGGCAAG |
| | CCAGACCAUCUGGAGCUACCUGAACUGGUACCAGCAGCGUCCCGGCAAGGCCCCCAAUCUGCUGAUCUACGCCGCCUCCAGCCUGCAGUCCGGAGUG |
| | CCCUCGAGGUUCAGCGGCCGGGGCAGCGGGACCGACUUCACCCUGACCAUCAGCUCGCUGCAGGCCGAGGACUUUGCCACCUACUACUGCCAACAG |
| | AGCUACUCAAUCCCCCAGACGUUUGGCCAGGGCACCAAGCUGGAGAUCAAGGCCAGCACCACAACCCCCGCGCCCCGGCCCCCCACCCCCGCCCCCACC |
| | AUCGCCAGCCAGCCGCUGAGCCUGAGGCCGGAAGCGUGCCGACCGGCCGCCGGGGGCGCCGUACACACCCGCGGACUCGACUUCGCCUGCGACAUAU |
| | AUAUCUGGGCCCCCCUGGCCGGCACCUGCGGCGUCCUGCUCCUGAGCCUGGUGAUCACCCUGUACUGCAACCACAGGAACGUCCGGUCCAAGCGUU |
| | CCCGCCUCCUGCACAGCGAUUACAUGAACAUGACCCCCCGACGCCCCGGCCCAACCCGAAAGCACUACCAGCCAUAUGCCCCCCCCAGGGACULUGQC |
| | GCCUAUCGAUCCAGGGUGAAGUUCAGCAGGUCAGCGGACGCGCCCGCCUACCAGCAGGGCCAGAACCAGCUGUAUAACGAGCUGAACCUCGGGGGG |
| | CGAGAGGAGUACGACGUGCUCGACAAGCGCCGGGGCAGGGACCCCGAAAUGGGAGGGAAGCCCCGCAGGAAAAACCCCCAGGAGGGACUCUACAAC |
| | GAGCUCCAAAAGGAUAAGAUGGCCGAAGCCUACAGCGAGAUCGGCAUGAAAGGGGAGAGGAGGCGGGGCAAGGGGCACGACGGCCUGUACCAGGG |
| | ACUGUCAACCGCCACCAAGGACACCUACGACGCCCUGCACAUGCAGGCCCUGCCGCCCAGGUAGUGAUGAGGCACAUUUGAAUAAAUUCUAUUACC |
| | AGUUCAAGCUUGGCGUAAUCAUGGUCAUAGCUG |
| 145 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUGAGGUGAAGCUGCAGGAGUCCGGCCCCGGGCUGGUCGCGCCCAGCCAGAGCCUCAGCGUGACCUGCACCGUGAGCGGCGU |
| | GAGCCUGCCCCGACUACGGCGUCAGCUGGAUCCGCCAGCCCCCCAGGAAGGGGCUGGAGUGGCUGGGCGUGAUCUGGGGCAGUGAGACGACCUAUU |
| | ACAACUCCGCCCUCAAGAGCCGCCUAACCAUCAUCAAGGACAACAGCAAGAGCCAGGUGUUCCUGAAGAUGAACAGCCUGCAGACCGACGACACCGC |
| | CAUCUACUACUGCGCCAAGCACUACUACUACGGGGGGUCCUACGCCAUGGACUACUGGGGGCAGGGCACCAGCGUGACCGUCAGCUCGGGGGGCGG |
| | CGGCAGCGGGGGAGGCGGGUCGGGGGGGGGGGGCAGCGACAUCCAGAUGACCCAGACCACCAGCUCCCUGAGCGCCUCCCUCGGCGACCGAGUCAC |
| | GAUCAGCUGCAGGGCCUCCCAGGACAUCAGCAAGUACCUCAACUGGUACCAGCAGAAGCCGGACGGGACCGUGAAGCUGCUGAUCUACCACACCUCC |
| | CGGCUGCACAGCGGCGUGCCCAGCCGGUUCAGCGGCUCCGGCAGCGGCACCGACUACAGCCUGACCAUCAGCAACCUGGAGCAGGAGGACAUCGCCA |
| | CCUACUUCUGCCAGCAGGGGAACACCCUGCCCUACACGUUCGGCGGCGGCACCAAGCUGGAGAUCAAGGCGGCCGCGAUAGAGGUAAUGUACCCUC |
| | CUCCUUACCUGGACAACGAGAAGUCGAACGGCACCAUCAUCCACGUCAAGGGCAAGCACCUCUGCCCCUCGCCCCUGUUCCCCGGCCCCUCCAAGCCC |
| | UUCUGGGUGCUGGUCGUGGUUGGUGGCGUCCUCGCCUGCUACUCCCUCCUCGUCACGGUGGCGUUCAUCAUCUUCUGGGUGCGGAGCAAGCGAA |
| | GCCGCCUCCUGCACAGCGACUACAUGAACAUGACGCCCCGCCGCCCCGGCCCGACCCGGAAGCACUACCAGCCCUACGCCCCGCCGCGCGACUUCGCG |
| | GCCUACCGCUCCCGGGUCAAGUUCUCCCGCAGCGCCGACGCCCCCGCGUACCAGCAGGGCCAGAACCAGCUCUACAACGAGCUCAACCUGGGGAGGC |
| | GCGAGGAGUACGACGUGCUUGACAAGCGCCGUGGCCGGGACCCUGAGAUGGGGGGUAAGCCGCGACGGAAGAACCCGCAGGAGGGCCUGUACAAC |
| | GAGCUGCAGAAGGACAAGAUGGCGGAGGCCUACAGCGAGAUCGGGAUGAAGGGGGAGCGGCGGCGUGGCAAGGGGCACGACGGCCUGUACCAGG |
| | GCCUCAGUACGGCCACCAAGGACACCUACGACGCCUUGCACAUGCAGGCCCUCCCCCCCCGCUAGUGAUGAGGCACAUUUGAAUAAAUUCUAUUACC |
| | AGUUCAAGCUUGGCGUAAUCAUGGUCAUAGCUG |
| 146 | GGGUCAAGAUUAGAGAACGGUCGUAGCAUUAUCGGAGGUUCUGCCACCAUGGCUCUCCCAGUGACCGCCCUGCUGCUGCCCCUAGCCCUCCUUUU |
| | GCACGCAGCGAGGCCUCAGGUCCAGCUCGUGGAGAGCGGCGGCGGCGUGGUGCAGCCCGGCCGAAGCCUCCGCCUAAGCUGCGCCGCCAGCGGGUU |
| | CACCUUCAGCAGCUACGGCAUGCACUGGGUACGGCAGGCCCCCGGCAAAGGACUGGAGUGGGUGGCGAUCAUAUACUACGACGGGAGCAAGAAAU |
| | ACUACGCCGACUCCGUCAAGGGCAGGUUUACCAUCAGCCGCGACAACAGCAAGAACACGCUGUACCUGCAGAUGAACAGCCUGAGGGCCGAGGACAC |
| | CGCCGUCUACUACUGUGCAAGAGAGCUAACCGGCGACGCGUUUGACAUCUGGGGGCAGGGCACCAAGGUGACCGUGUCCUCCGGGGGCGGGGGCA |
| | GCGGCGGCGGCGGCUCCGGCGGCGGCGGGUCAGGCGGCGGCGGCAGCGAGAUAGUGCUGACCCAGACCCCAUCCAGCCUGUCCGCCUCCCUGGGCG |
| | ACCGCGUGACGAUCAGCUGCCGGGCCAGCCAGUCGAUCGGCAGCAGCCUACACUGGUACCAGCAGAAGCCGGACCAGUCCCCCAAGCUGCUCAUCAA |
| | GUACGCCUCCCAGUCAUUCUCCGGGGUGCCCUCAAAGUUCAUCGGGAGCGGGUCCGGCACGGACUUCACACUCACCAUCAACAGCCUGGAGGCCGA |
| | GGACGCCGCGACCUUUUACUGCCACCAAAGCAGCACCCUCCCCUACACCUUCGGCCAGGGCACAAAGCUGGAGAUCAAGGCGGCCGCGAUAGAGGUA |
| | AUGUACCCUCCUCCUUACUUGGACAACGAGAAGUCGAACGGCACCAUCAUCCACGUCAAGGGCAAGCACCUCUGCCCGUCGCCGCUGUUCCCGGGCC |
| | CGUCCAAGCCCUUCUGGGUGCUGGUCGUGGUUGGUGGCGUCCUCGCCUGCUACUCCCUCCUCGUCACGGUGGCGUUCAUCAUCUUCUGGAAGCGC |
| | GGGCAGGAAGAAGCUCCUGUACAUCUUCAAGCAGCCGUUCAUGCGCCCGGUGCAGACGACCCAGGAGGAGGACGGGUGCAGCUGCCGGUUCCCGGA |
| | GGAGGAGGAGGGCGGCUGCGAGCUGCGGGUCAAGUUCUCCCGCAGCGCCGACGCGCCCGCGUACCAGCAGGGCCAGAACCAGCUCUACAACGAGCU |
| | CAACCUGGGGAGGCGCGAGGAGUACGACGUGCUUGACAAGCGCCGUGGCCGGGACCCUGAGAUGGGCGGUAAGCCGCGACGGAAGAACCCGCAGG |
| | AGGGCCUGUACAACGAGCUGCAGAAGGACAAGAUGGCGGAGGCCUACAGCGAGAUCGGGAUGAAGGGCGAGCGGCGGCGUGGCAAGGGCCACGAC |
| | GGCCUGUACCAGGGCCUCAGUACGGCCACCAAGGACACCUACGACGCCUUGCACAUGCAGGCCCUCCCGCCGCGCUAGUGAUGAGGCACAUUUGAA |
| | UAAAUUCUAUUACCAGUUCAAGCUUGGCGUAAUCAUGGUCAUAGCUG |
| 147 | GGACAGAUCGCCUGGAGACGCCAUCCACGCUGUUUUGACCUCCAUAGAAGACACCGGGACCGAUCCAGCCUCCGCGGCCGGGAACGGUGCAUUGGA |
| | ACGCGGAUUCCCCGUGCCAAGAGUGACUCACCGUCCUUGACACGAUGGCCCUGCCCGUGACAGCACUGCUGCUGCCAUUAGCUCUGCUGCUGCACG |
| | CCGCUAGACCACAGGUGCAGCUGGUGGAGAGCGGAGGAGGAGUGGUCCAGCCCGGCAGAUCCCUCAGACUGAGCUGCGCCGCUUCCGGCUUCACCU |
| | UCAGCUCCUACGGCAUGCACUGGGUGAGACAGGCUCCAGGCAAGGGCCUGGAAUGGGUCGCAAUCAUCUACUACGAUGGGUCUAAGAAGUAUUAC |
| | GCCGACUCCGUGAAGGGGAGGUUCACAAUCAGCAGAGACAAUAGCAAGAAUACUCUGUAUCUGCAGAUGAACUCCCUGAGAGCCGAGGACAGCU |
| | GUGUACUACUGCGCCCGCGAGCUGACUGGCGAUGCAUUCGACAUCUGGGGGCAGGGGACCAAGGUGACCGUGUCCUCUGGCGGCGGAGGCUCUG |
| | GAGGAGGAGGCUCUGGAGGAGGGGGCAGCGGCGGAGGAGGAAGUGAGAUCGUGCUGACCCAGACCCCUAGCAGCCUGAGCGCCUCCCUGGGCGAC |
| | AGAGUGACCAUCAGCUGCCGGGCCUCUCAGAGCAUCGGCAGCUCCCUGCACUGGUACCAGCAGAAGCCUGACCAGAGCCCAAAGCUGCUGAUCAAG |
| | UACGCCAGCCAGAGCUUUAGCGGCGUGCCCAGCAAGUUCAUCGGCAGCGGCUCCGGCACCGAUUUCACCCUGACCAUCAAUUCCCUGGAGGCCGAA |
| | GAUGCUGCCACCUUCUACUGCCACCAGAGCAGCACCCUGCCCUACACCUUCGGCCAGGGCACCAAGCUGGAGAUCAAAGCCGCCGCCAUCGAAGUGA |
| | UGUACCCCCCCCCCACCUGGACAACGAGAAGUCCAACGGCACCAUCAUCCACGUGAAAGGCAAGCACCUGUGCCCCUCCCCCCUGUUCCCUGGACCU |
| | UCCAAGCCUUUCUGGGUGCUGGUGGUGGUGGGCGGCGUGCUGGCCUGCUACAGCCUGCUGGUGACAGUGGCCUUCAUCAUCUUCUGGGUGAGAA |
| | GCAAGCGGUCCCGGCUGCUGCACAGCGACUACAUGAACAUGACCCCCAGAAGACCCGGCCCUACCAGAAAGCACUACCAGCCCUACGCCCCCCCCCAGA |
| | GAUUUCGCCGCCUACAGAAGCCGGUGAAGUUCAGCAGAAGCGCCGACGCCCCUGCCUACCAGCAGGGCCAGAAUCAGCUGUACAACGAGCUGAAC |
| | CUGGGCAGAAGAGAGGAGUACGACGUGCUGGACAAGAGAAGAGGCAGAGAUCCCGAGAUGGGCGGCAAGCCCAGACGGAAGAACCCUCAGGAGGG |
| | CCUGUACAAUGAGCUGCAGAAGGACAAGAUGGCCGAGGCCUACAGCGAGAUCGGCAUGAAGGGCGAGAGAAGAAGAGGCAAGGGCCACGACGGCC |
| | UGUACCAGGGCCUGAGCACCGCCACAAAGGACACCUACGAUGCCCUGCACAUGCAGGCCCUGCCCCCUAGAUAGUGAUAACGGGUGGCAUCCCUGU |
| | GACCCCUCCCCAGUGCCUCUCCUGGCCCUGGAAGUUGCCACUCCAGUGCCCACCAGCCUUGUCCUAAUAAAAUUAAGUUGCACU |
| 148 | GGACAGAUCGCCUGGAGACGCCAUCCACGCUGUUUUGACCUCCAUAGAAGACACCGGGACCGAUCCAGCCUCCGCGGCCGGGAACGGUGCAUUGGA |
| | ACGCGGAUUCCCCGUGCCAAGAGUGACUCACCGUCCUUGACACGAUGGCCCUGCCCGUGACAGCACUGCUGCUGCCAUUAGCUCUGCUGCUGCACG |
| | CCGCUAGACCACAGGUGCAGCUGGUGGAGAGCGGAGGAGGAGUGGUCCAGCCCGGCAGAUCCCUCAGACUGAGCUGCGCCGCUUCCGGCUUCACCU |
| | UCAGCUCCUACGGCAUGCACUGGGUGAGACAGGCUCCAGGCAAGGGCCUGGAAUGGGUCGCAAUCAUCUACUACGAUGGGUCUAAGAAGUAUUAC |
| | GCCGACUCCGUGAAGGGGAGGUUCACAAUCAGCAGAGACAAUAGCAAGAAUACUCUGUAUCUGCAGAUGAACUCCCUGAGAGCCGAGGACACAGCU |
| | GUGUACUACUGCGCCCGCGAGCUGACUGGCGAUGCAUUCGACAUCUGGGGGCAGGGGACCAAGGUGACCGUGUCCUCUGGCGGCGGAGGCUCUG |
| | GAGGAGGAGGCUCUGGAGGAGGGGGCAGCGGCGGAGGAGGAAGUGAGAUCGUGCUGACCCAGACCCCUAGCAGCCUGAGCGCCUCCCUGGGCGAC |
| | AGAGUGACCAUCAGCUGCCGGGCCUCUCAGAGCAUCGGCAGCUCCCUGCACUGGUACCAGCAGAAGCCUGACCAGAGCCCAAAGUCGCUGAUCAAG |
| | UACGCCAGCCAGAGCUUUAGCGGCGUGCCCAGCAAGUUCAUCGGCAGCGGCUCCGGCACCGAUUUCACCCUGACCAUCAAUUCCCUGGAGGCCGAA |
| | GAUGCUGCCACCUUCUACUGCCACCAGAGCAGCACCCUGCCCUACACCUUCGGCCAGGGCACCAAGCUGGAGAUCAAAGCCGCCGCCAUCGAAGUGA |
| | UGUACCCCCCCCCCUACCUGGACAACGAGAAGUCCAACGGCACCAUCAUCCACGUGAAAGGCAAGCACCUGUGCCCCUCCCCCCUGUUCCCUGGACCU |
| | UCCAAGCCUUUCUGGGUGCUGGUGGUGGUGGGCGGCGUGCUGGCCUGCUACAGCCUGCUGGUGACAGUGGCCUUCAUCAUCUUCUGGAAGCGGG |
| | GCAGAAAGAAGCUGCUGUACAUCUUCAAGCAGCCCUUCAUGCGGCCCGUGCAGACCACCCAGGAGGAGGAUGGCUGCAGCUGCAGAUUUCCUGAG |
| | GAGGAGGAGGGCGGCUGCGAGCUGCGGGUGAAGUUCAGCAGAAGCGCCGACGCCCCUGCCUACCAGCAGGGCCAGAAUCAGCUGUACAACGAGCU |
| | GAACCUGGGCAGAAGAGAGGAGUACGACGUGCUGGACAAGAGAAGAGGCAGAGAUCCCGAGAUGGGCGGCAAGCCCAGACGGAAGAACCCUCAGG |
| | AGGGCCUGUACAAUGAGCUGCAGAAGGACAAGAUGGCCGAGGCCUACAGCGAGAUCGGCAUGAAGGGCGAGAGAAGAAGAGGCAAGGGCCACGAC |
| | GGCCUGUACCAGGGCCUGAGCACCGCCACAAAGGACACCUACGAUGCCCUGCACAUGCAGGCCCUGCCCCCAGAUAGUGAUAACGGGUGGCAUCCC |
| | UGUGACCCCUCCCCAGUGCCUCUCCUGGCCCUGGAAGUUGCCACUCCAGUGCCCACCAGCCUUGUCCUAAUAAAUUAAGUUGCAUC |
| 149 | SQFRVSPLDRTWNLGETVELKCQVELSNPTSGCSWLFQPRGAAASPTFLLYLSQNKPRAAEGLDTQRFSGERLCDTFVETLS |
| | DFRRENEGYYFCSALSNSIMYFSHFVPVFLPAKPTTTPAPRPPTPAPTIASQPLSLRPEACRPAAGGAVRTRGLDFACPPCPAPE |
| | YKCKVSNKALPAPIEKTISKAKGQPREPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGHHHHHH |
| 150 | SQFRVSPLDRTWNLGETVELKCQVLLSNPTSGCSWLFQPRGAAASPTFLLYLSQNKPKAAEGLDTQRESGKRLGDTFVETES |
| | DFRRENEGYYFCSALSNSIMYFSHFVPVFLPAKPTTTPAPRPPTPAPTIASQPLSLRPEACRPAAGGAVHTRGLDFAC |
| 151 | PPCPAPELLGGPSVFLFPPKPKDTIMISRTPEVTCVVVDVSHEDPEVKFNWYVDGVEVHNAKTKPREEQYNSTYRVVSVLTVLHQDWINGKEYKCKVSNKALPAP |
| | IEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYT |
| | QKSLSLSPG |
| 152 | HHHHHH |
| 153 | LQQTPAYIKVQTNKMVMLSCEAKISLSNMRIYWLRQRQAPSSDSHHEFLALWDSAKGTINGEEVEQERIAVFRDASRFILNE |
| | TSVKPEDSGIYFCMIVGSPELTFGKGTQLSVVDFLPTTAQPTKKSTLKKRVCRLPRPETQKGPLCPPCPAPELLGGPSVFLFPPK |
| | PIEKTISKAKGQPREPQVYTLPPSRDELTKNQVSLTCLVKGFYPSDIAVEWESNGQPENNYKTTPPVLDSDGSFFLYSKLTVDKSRW |
| | QQGNVFSCSVMHEALHNHYTQKSLSLSPGWSHPQFEKGGGSGGGSGGSSAWSHPQFEK |
| 154 | WISHPQFEKGGGSGGGSGGSSAWISHPQFEK |
| 155 | NQFRVSPLGRTWNLGETVELKCQVLLSNFTSGCSWLFQPRGTAARPTFLLYLSQNKPKAAEGLDTQRFSGERLGDTFVETL |
| | RDFRQENEGYYFCSALSNSIMYFSHFVPVFLPAKPTTTPAPRPPTPAPTTASQPLSLRPEACRPAAGGSVNTRGLDFACPPCPA |
| | PELLGGPSVFLEPPKPKDTLMISRTPEVTCVVVDVSHEDPEVKENWYVDGVEVHNAKTKPREEQYNSTYRVVSVLTVLHQDWLNG |
| | KEYKCKVSNKALPAPIEKTISKAKGQPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGHHHHHH |
| 156 | NQFRVSPLGRTWNLGETVELKCQVLLSNPTSGCSWLFQPRGTAARPTFLLYLSQNKPKAAEGLDTQRFSGKRLGDTFVLTL |
| | RDFRQENEGYYFCSALSNSIMYFSHFVPVFLPAKPTTTPAPRPPTPAPTTASQPLSLRPEACRPAAGGSVNTRGLDFAC |
| 157 | LQQTPAYIQVQTNKMAMLSCEAKISLGNMRIYWLRQRQAPSSDSHHEFLALWDSTKGTVHIGEEVAQERIAVFRDASRFILN |
| | LTSVKPEDSGIYFCMIIGSPELTFGKGTQLSVVDFLPTTAQPTKKSTPKKRGCRLPRPATQKGPLCPPCPAPELLGGPSVFLFPP |
| | APIEKTISKAKGQPREPQVYTLPPSRDELTKNQVSLTCLVKGFYPSDIAVEWESNGQPENNYKTTPPVLDSDGSFFLYSKLTVDKSR |
| | WQQGNVFSCSVMHEALHNHYTQKSLSLSPGWSHPQFEKGGGSGGGSGGSSAWSHPQFEK |
| 158 | LQQTPAYIQVQTNKMAMLSCEAKISLGNMRIYWLRQRQAPSSDSHHEFLALWDSTKGTVHGEEVAQEKIAVFRDASRFILN |
| | LTSVKPEDSGIYFCMHGSPELTFGKGTQLSVVDFLPTTAQPTKKSTPKKRGCRLPRPATQKGPLC |
| 159 | AGGAAAUAAGAGAGAAAAGAAGAGUAAGAAGAAUAUAAGAGCCACCAUGGAGACCGACACCCUCCUGCUGUGGGUGCUGCUGCUUUGGGUGCCC |
| | GGGAGCACGGGGGACUACAAGGCGAAGGAGGUGCAGCUGCAGCAGAGCGGGCCGAGCUGGUGAAGCCGGGCGCCAGCGUCAAGAUGAGCUGCAA |
| | GGCUAGCGGGUACACCUUCACCUCCUACAACAUGCACUGGGUUAAGCAGACCCCCGGGCAGGGCCUGGAGUGGAUCGGGGCCAUCUACCCAGGGAA |
| | CGGGGACACCUCCUACAACCAGAAGUUCAAGGGCAAGGCCACGCUCACUGCGGACAAGUCCUCCAGCACAGCCUACAUGCAGCUGAGCAGCCUGACG |
| | AGCGAGGACUCGGCGGACUACUACUGCGCACGCAGCAACUACUACGGGUCCAGCUACUGGUUCUUCGACGUCUGGGGCGCGGGGACGACGGUCAC |
| | CGUCAGCUCCGGCGGGGGCAGCGGCGGCGGGAGCGGGGGGGGCGGAGCAGCAGACAUGUGCUGACCCAGUCCCCCGCCAUCCUGUCUCGCGAGCCC |
| | CGGGGAGAAGGUCACCAUGACGUGCCGGGCCAGCUCAAGUGUGAACUACAUGGACUGGUACCAGAAGAAGCCGGGAUCCUCCCCCAAGCCCUGGAU |
| | UUACGCGACGUCCAACCUCGCGAGCGGGGUCCCUGCGCGCUUCUCUGGCUCCGGGUCGGGGACCAGCUACAGCCUCACCAUCAGCCGCGUGGAGGC |
| | CGAGGACGCGGCGACCUACUACUGCCAGCAGUGGAGCUUCAACCCCCCGACGUUCGGCGGGGGGACCAAGCUGGAGAUCAAGGGAGGGGGCGGAU |
| | CCGCGGCCGCGAUAGAGGUAAUGUACCCUCCUCCUUACCUGGACAACGAGAAGUCGAACGGCACCAUCAUCCACGUCAAGGGCAAGCACCUCUGCCC |
| | CUCGCCCCUGUUUCCCCGGCCCCUCCAAGCCCUUCUGGGUGCUGGUCGUGGUUGGUGGCGUCCUCGCCUGCUACUCCCUCCUCGUCACGGUGGCGUU |
| | CAUCAUCUUCUGGGUGCGGAGCAAGCGAAGCCGCCUCCUGCACAGCGACUACAUGAACAUGACGCCCCGCCGCCCCGGCCCGACCCGGAAGCACUAC |
| | CAGCCCUACGCCCCGCCGCGCGACUUCGCGGCCUACCGCUCCCGGGUCAAGUUCUCCCGCAGCGCCGAGCCCCCCGCGUACCAGCAGGGCCAGAACCA |
| | GCUCUACAACGAGCUGAACCUGGGGAGGCGCGAGGAGUACGACGUGCUUGACAAGCGCCGUGGCCGGGACCCUGAGAUGGGGGGUAAGCCGCGAC |
| | GGAAGAACCCGCAGGAGGGCCUGUACAACGAGCUGCAGAAGGACAAGAUGGCGGAGGCCUACAGCGAGAUGGGAUGAAGGGGGAGCGGCGGCGU |
| | GGCAAGGGGCACGACGGCCUGUACCAGGGCCUCAGUACGGCCACCAAGGACACCUACGACGCCUUGCACAUGCAGGCCCUCCCCCCCCGCUAGUGAU |
| | GAGCGGCCGCUUAAUUAAGCUGCCUUCUGCGGGGCUUGCCUUCUGGCCAUGCCCUUCUUCUCUCCCUUGCACCUGUACCUCUUGGUCUUUGAAUA |
| | AAGCCUGAGUAGGAAG |
| 160 | EVQLVESGGGLVQAGGSLRLSCAASGFTFDDYAIGWFRQAPGKEREEVSCIRTYDGNTYYIDSVKGRFTISSDNAKNTVYLQMNSLKPEDTAAYYCAAGSYYACAK |
| | YSRPDPSENHVDMDYWGKGTLVTVSS |
| 161 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAK |
| | YSRPDPSEGHVDLDYWGQGTLVTVSS |
| 162 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAE |
| | YSRPDPSEGHVDLDYWGQGTLVTVSS |
| 163 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAY |
| | YSRPDPSEGHVDLDYWGQGTLVTVSS |
| 164 | EVQLVESGGGVVQPGGSLRLSCAASGFTFEDYAIGWFREVDEQTYYADSVKGRFTISRDNAKNTVDLQMNSLRPEDTALYYCAAGSYYACA |
| | KYSRPDPSEGHVDLDYWGQGTLVTVSS |
| 165 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | EYSRPDPSEGHVDLDYWGQGTLVTVSS |
| 166 | EVQLVESGGGVVQPGGSLRLSCAASGFTFEDYAIGWFRQAPGKEREEVSCIRTYDEQTYYADSVKGRFTISRDNAKNTVDLQMNSLRPEDTALYYCAAGSYYACA |
| | YYSRPDPSEGHVDLDYWGQGTLVTVSS |
| 167 | EVQLVESGGGVVQPGGSLRLSCAASGFTFEDYAIGWFRQAPGKEREEVSCIRTYDEQTYYADSVKGRFTISRDNAKNTVSLQTVINSLRPEDTALYYCAAGSYYACAK |
| | YSRPDPSEGHVDLDYWGQGTLVTVSS |
| 168 | EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAE |
| | YSRPDPSEGHVDLDYWGQGTLVTVSS |
| 169 | EVQLVESGGGVVQPGGSLRLSCAASGFTFEDYAIGWFRQAPGKEREEVSCIRTYDEQTYYADSVKGREFTISRDNAKNTVSLQMINSIRPEDTALYYCAAGSYYACAY |
| | YSRPDPSEGHVDLDYWGQGTLVTVSS |
| 170 | EVQLVESGGGLVQAGGSLRLSCAASGFTEDDYAIGWFRQAPGKEREEVSCIRTYDGNTYIDSVKGRFTISSDNAKNTVYLQMNSLKPEDTAAYYCAAGSYYACAK |
| | YSRPDPSENHVDMDYWGKGTLVTVSS |
| 171 | EVQLVESGGGVVQPGGSLRLSCAASGFTFEDYAIGWFRQKEREEVSCIRTYDEQTYYADSVKGRFTISRDNAKNTVYLQMNSLRPEDTALYYCAAGSYYACAK |
| | YSRPDPSEGHVDLDYWGQGTLVTVSS |
| 172 | EVQLVESGGGVVQPGGSLRLSCAASGFTFEDYAIGWFRQAPGKEREEVSCIRTYDEQTYYADSVKGRFTISRQNAKNTVYLQIVINSLRPEDTALYYCAAGSYYACAE |
| | YSRPDPSEGHVDLDYWGQGTLVTVSS |
| 173 | EVQLVESGGGVVQPGGSLRLSCAASGFTFEDYAIGWFRQAPGKEREEVSCIRTYDEQTYYADSVKGRFTISRDNAKNTVYLQMNSLRPEDTALYYCAAGSYYACAY |
| | YSRPDPSEGHVDLDYWGQGTLVTVSS |
| 174 | EVQLVESGGGVVQPGGSLRLSCAASGFTFEDYAIGWFRQAPGKEREEVSCIRTYDEQTYVADSVKGRFTISRDNAKNTVDLQMNSLRPEDTALYYCAAGSYYACA |
| | KYSRPDPSEGHVDLDYWGQGTLVTVSS |
| 175 | EVQLVESGGGVVQPGGSLRLSCAASGFTFEDYAIGWFRQAPGKEREEVSCIRTYDEQADSVKGRFTISRDNAKNTVDLQMNSLRPEDTALYYCAAGSYYACA |
| | EYSRPDPSEGHVDLDYWGQGTLVTVSS |
| 176 | EVQLVESGGGVVQPGGSIRISCAASGFTFEDYAIGWFRQAPGKEREEVSCIRTYDEQTYYADSVKGRFTISRDNAKNTVDLQMNSLRPEDTALYYCAAGSYYACA |
| | YYSRPDPSEGHVDLDYWGQGTLVTVSS |
| 177 | EVQLVESGGGVVQPGGSLRLSCAASGFTFEDYAIGWFRQAPGKEREEVSCIRTYDEQTYYADSVKGRFTISRDNAKNTVSLQMNSLRPEDTALYYCAAGSYYACAK |
| | YSRPDPSEGHVDLDYWGQGTLVTVSS |
| 178 | EVQLVESGGGVVQPGGSLRLSCAASGFTFEDYAIGWFRQAPGKEREEVSCIRTYDEQTYYADSVKGRFTISRDNAKNTVSLQNINSLRPEDTALYYCAAGSYYACAE |
| | YSRPDPSEGHVDLDYWGQGTLVTVSS |
| 179 | EVQLVESGGGVVQPGGSLRLSCAASGFTFEDYAIGWFRQAPGKEREEVSCIRTYDEQTYYADSVKGRFTISRDNAKNTVSLQVINSLRPEDTALYYCAAGSYYACAY |
| | YSRPDPSEGHVDLDYWGQGTLVTVSS |
| 180 | EVQLVESGGGLVQAGGSLRLSCAAS |
| 187 | EVQLVESGGGVVQPGGSLRLSCAAS |
| 194 | EVQLVESGGGVVQPGGSLRLSCAAS |
| 201 | EVQLVESGGGVVQPGGSURLSCAAS |
| 208 | EVQLVESGGGVVQPGGSLRLSCAAS |
| 215 | EVQLVESGGGVVQPGGSLRLSCAAS |
| 222 | EVQLVESGGGVVQPGGSLRLSCAAS |
| 229 | EVQLVESGGGVVQPGGSLRLSCAAS |
| 236 | EVQLVESGGGVVQPGGSLRLSCAAS |
| 243 | EVQLVESGGGVVQPGGSLRLSCAAS |
| 181 | GFTFDDYAIG |
| 188 | GFTFEDYAIG |
| 195 | GFTFEDYAIG |
| 202 | GFTFEDYAIG |
| 209 | GFTFEDYAIG |
| 216 | GFTFEDYAIG |
| 223 | GFTFEDYAIG |
| 230 | GFTFEDYAIG |
| 237 | GFTFEDYAIG |
| 244 | GFTFEDYAIG |
| 182 | WFRQAPGKEREEVS |
| 189 | WFRQAPGKEREEVS |
| 196 | WFRQAPGKEREEVS |
| 203 | WFRQAPGKEREEVS |
| 210 | WFRQAPGKEREEVS |
| 217 | WFRQAPGKEREEVS |
| 224 | WFRQAPGKEREEVS |
| 231 | WFRQAPGKEREEVS |
| 238 | WFRQAPGKEREEVS |
| 245 | WFRQAPGKEREEVS |
| 183 | CIRTYDGNTY |
| 190 | CIRTYDEQTY |
| 197 | CIRTYDEQTY |
| 204 | CIRTYDEQTY |
| 211 | CIRTYDEQTY |
| 218 | CIRTYDEQTY |
| 225 | CIRTYDEQTY |
| 232 | CIRTYDEQTY |
| 239 | CIRTYDEQTY |
| 246 | CIRTYDEQTY |
| 184 | YIDSVKGRFTISSDNAKNTVYLQMNSLKPEDTAAYYCAA |
| 191 | YADSVKGRFTISRDNAKNTVYLQMNSLRPEDTALYYCAA |
| 198 | YADSVKGRFTISRDNAKNTVYLQMNSLRPEDTALYYCAA |
| 205 | YADSVKGRFTISRDNAKNTVYLQMNSLRPEDTALYYCAA |
| 212 | YADSVKGRFTISRDNAKNTVDLQMNSLRPEDTALYYCAA |
| 219 | YADSVKGRFTISRDNAKNTVDLQMNSLRPEDTALYYCAA |
| 219 | VADSVKGRFTISRDNAKNTVDLQMNSLRPEDTALYYCAA |
| 226 | VADSVKGRFTISRDNAKNTVDLQMNSLRPEDTALYYCAA |
| 233 | YADSVKGRFTISRDNAKNTVSLQMNSLRPEDTALYYCAA |
| 240 | YADSVKGRFTISRDNAKNTVSLQMNSLRPEDTALYYCAA |
| 247 | YADSVKGRFTISRDNAKNTVSLQMNSLRPEDTALYYCAA |
| 185 | GSYYACAKYSRPDPSENHVDMDY |
| 192 | GSYYACAKYSRPDPSEGHVDLDY |
| 199 | GSYYACAEYSRPDPSEGHVDLDY |
| 206 | GSYYACAYYSRPDPSEGHVDLDY |
| 213 | GSYYACAKYSRPDPSEGHVDLDY |
| 220 | GSYYACAEYSRPDPSEGHVDLDY |
| 227 | GSYYACAYYSRPDPSEGHVDLDY |
| 234 | GSYYACAKYSRPDPSEGHVDLDY |
| 241 | GSYYACAEYSRPDPSEGHVDLDY |
| 248 | GSYYACAYYSRPDPSEGHVDLDY |
| 186 | WGKGTLVTVSS |
| 193 | WGQGTLVTVSS |
| 200 | WGQGTLVTVSS |
| 207 | WGQGTLVTVSS |
| 214 | WGQGTLVTVSS |
| 221 | WGQGTLVTVSS |
| 228 | WGQGTLVTVSS |
| 235 | WGQGTLVTVSS |
| 242 | WGQGTLVTVSS |
| 249 | WGQGTLVTVSS |
| 250 | EVQLVESGGGLVQAGGSLRLSCAASGFTFD |
| 257 | EVQLVESGGGVVQPGGSLRLSCAASGFTFE |
| 264 | EVQLVESGGGVVQPGGSLRLSCAASGETFE |
| 271 | EVQLVESGGGVVQPGGSLRLSCAASGFTFE |
| 278 | EVQLVESGGGVVQPGGSLRLSCAASGFTFE |
| 285 | EVQLVESGGGVVQPGGSLRLSCAASGFTFE |
| 292 | EVQLVESGGGVVQPGGSURLSCAASGFTFE |
| 299 | EVQLVESGGGVVQPGGSLRLSCAASGFTFE |
| 306 | EVQLVESGGGVVQPGGSLRLSCAASGFTFE |
| 313 | EVQLVESGGGVVQPGGSLRLSCAASGFTFE |
| 251 | DYAIG |
| 258 | DYAIG |
| 265 | DYAIG |
| 272 | DYAIG |
| 279 | DYAIG |
| 286 | DYAIG |
| 293 | DYAIG |
| 300 | DYAIG |
| 307 | DYAIG |
| 314 | DYAIG |
| 252 | WFRQAPGKEREEVS |
| 259 | WFRQAPGKEREEVS |
| 266 | WFRQAPGKEREEVS |
| 273 | WFRQAPGKEREEVS |
| 280 | WFRQAPGKEREEVS |
| 287 | WFRQAPGKEREEVS |
| 294 | WFRQAPGKEREEVS |
| 301 | WFRQAPGKEREEVS |
| 308 | WFRQAPGKEREEVS |
| 315 | WFRQAPGKEREEVS |
| 253 | CIRTYDGNTYYIDSVKG |
| 260 | CIRTYDEQTYYADSVKG |
| 267 | CIRTYDEQTYYADSVKG |
| 274 | CIRTYDEQTYYADSVKG |
| 281 | CIRTYDEQTYYADSVKG |
| 288 | CIRTYDEQTYYADSVKG |
| 295 | CIRTYDEQTYYADSVKG |
| 302 | CIRTYDEQTYYADSVKG |
| 309 | CIRTYDEQTYYADSVKG |
| 316 | CIRTYDEQTYYADSVKG |
| 254 | RFTISSDNAKNTVYLQMNSLKPEDTAAYYCAA |
| 261 | RFTISRQNAKNTVYLQMNSLRPEDTALYYCAA |
| 268 | RFTISRDNAKNTVYLQMNSLRPEDTALYYCAA |
| 275 | RFTISRDNAKNTVYLQMNSLRPEDTALYYCAA |
| 282 | RFTISRDNAKNTVDLQMNSLRPEDTALYYCAA |
| 289 | RFTISRDNAKNTVDLQMNSLRPEDTALYYCAA |
| 296 | RFTISRDNAKNTVDLQMNSLRPEDTALYYCAA |
| 303 | RFTISRDNAKNTVSLQMNSLRPEDTALYYCAA |
| 310 | RFTISRDNAKNTVSLQMNSLRPEDTALYYCAA |
| 317 | RFTISRDNAKNTVSLQMNSLRPEDTALYYCAA |
| 253 | GSYYACAKYSRPDPSENHVDMDY |
| 262 | GSYYACAKYSRPDPSEGHVDLDY |
| 269 | GSYYACAEYSRPDPSEGHVDLDY |
| 276 | GSYYACAYYSRPDPSEGHVDIDY |
| 283 | GSYYACAKYSRPDPSEGHVDIDY |
| 290 | GSYYACAEYSRPDPSEGHVQLDY |
| 297 | GSYYACAYYSRPDPSEGHVDLDY |
| 304 | GSYYACAKYSRPDPSEGHVDLDY |
| 311 | GSYYACAEYSRPDPSEGHVDLDY |
| 318 | GSYYACAYYSRPDPSEGHVDLDY |
| 256 | WGKGTLVTVSS |
| 263 | WGQGTLVTVSS |
| 270 | WGQGTLVTVSS |
| 277 | WGQGTLVTVSS |
| 284 | WGQGTLVTVSS |
| 291 | WGQGTLVTVSS |
| 298 | WGQGTLVTVSS |
| 305 | WGQGTLVTVSS |
| 312 | WGQGTLVTVSS |
| 319 | WGQGTLVTVSS |
| 320 | EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSS |
| | QGTLVTVSS |
| 321 | EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSS |
| | QGTLVTVSS |
| 322 | EVQLVESGGGVVQPGNSLRLSCAASGFTFSSFGMSWVRQAPEWVSSISGSSDTLYADSVKGRFTISRDNAKTTLYLQMNSLRPEDTATYYCTIGGSLSRSS |
| | QGTLVTVSSA |
| 323 | EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSS |
| | QGTLVTVSSA |
| 324 | EVQLVESGGGLVQPGNSLRLSCAASGFTFSSFVGMSWVRQAPGKGLEWVSSISGSGSDTLYADSVKGRFTISRDNAKTTLYLQMNSLRPEDTAVYYCTIGGSLSRSS |
| | QGTLVTVSS |
| 325 | EVQLVESGGGLVQPGNSLRLSCAASGFTFSSFGMSWVRQAPGKGLEWVSSISGSGSDTLYADSVLGRFTISRDNAKTTLYLQMNSLRPEDTAVYYCTIGGSLSRSS |
| | QGTLVKVSSA |
| 326 | EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSS |
| | QGTLVTVSS |
| 327 | EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSS |
| | QGTLVTVSSA |
| 328 | EVQLVESGGGVVQPGNSLRLSCAASGFTFSSFGMSWVRQAPGKGLEWVSSISGSGSDTLYADSVKGRFTISRDNAKTTLYQMNSLRPEDTALYYCTIGGSLSSRSS |
| | QGTLVTVSSAA |
| 329 | EVQLVESGGGVVQPGNSLRLSCAASGFTFSSFGMSWVRQAPGKGLEWVSSISGSGSGTLYADSVKGRFTISRDNAKTTLYLQMNSLRPEDTALYYCTIGGSLSRSS |
| | QGTLVTVSSAAA |
| 330 | EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSS |
| | QGTLVTVSSG |
| 331 | EVQLVESGGGVVQPGNSLRLSCAASGFTFSSFGMSWVRQAPGKGLEWVSSISGSGSGTLYADSVKGRFTISRDNAKTTLYLQMNSLRPEDTALYYCTIGGSLSRSS |
| | QGLTVTVSSGG |
| 332 | EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSS |
| | QGTLVTVSSGGG |
| 333 | EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSS |
| | QGTLVTVSSA |
| 334 | EVQLVESGGGVVQPGGSLRLSCAASGLTFSSYAMGWFRQAPGKERERVVSISRGGGYTYYADSVKGRFTISRDNSENTVYLQMNSLRPEDTALYYCAAARYWAT |
| | GSEYEFDYWGQGTLVTVSS |
| 335 | EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSS |
| | QGTLVTVSS |
| 336 | AAA |
| 337 | GGGGS |
| 338 | SGGSGGS |
| 339 | GGGGSGGS |
| 340 | GGGGSGGGS |
| 341 | GGGGSGGGGS |
| 342 | GGGGSGGGGGGGGS |
| 343 | GGGGSGGGGSGGGGSGGS |
| 344 | GGGGSGGGGSGGGGSGGGGS |
| 345 | GGGGSGGGGSGGGGSGGGGSGGGGS |
| 346 | GGGGSGGGGSGGGGSGGGGSGGGGSGGGGS |
| 347 | GGGGSGGGGSGGGGSGGGGSGGGGSGGGGSGGGGS |
| 348 | GGGGSGGGGSGGGGSGGGGSGGGGSGGGGSGGGGSGGGGS |
| 349 | EPKSCDKTHTCPPCP |
| 350 | GGGGSGGGSEPKSCDKTHTCPPCP |
| 351 | EPKTPKPQPAAA |
| 352 | ELKTPLGQTTHTCPRCPEPKSCDTPPPCPRCPEPKSCDTPPPCPRCPEPKSCDTPPPCPRCP |
| 353 | VTVSS(X)n |
| 354 | VKVSS |
| 355 | VQVSS |
| 356 | VTVKS |
| 357 | VTVQS |
| 358 | VKVKS |
| 359 | VKVQS |
| 360 | VQVKS |
| 361 | VQVQS |
| 362 | VTVSSA |
| 363 | VKVSSA |
| 364 | VQVSSA |
| 365 | TVTVKSA |
| 366 | TVTVQSA |
| 367 | VKVKSA |
| 368 | VKVQSA |
| 369 | VQVKSA |
| 370 | VQVQSA |
| 371 | VTVSS |
| 372 | KERE |
| 373 | KQRE |
| 374 | GLEW |
| 375 | KEREL |
| 376 | KEREF |
| 377 | KQREL |
| 378 | KQRER |
| 379 | KEREG |
| 380 | KQREW |
| 381 | KQREG |
| 382 | TERE |
| 383 | TEREL |
| 384 | TQRE |
| 385 | TQREL |
| 386 | KECE |
| 387 | KECEL |
| 388 | KECER |
| 389 | KQCE |
| 390 | KQCEL |
| 391 | RERE |
| 392 | REREG |
| 393 | RQRE |
| 394 | RQREL |
| 395 | RQREF |
| 396 | RQREW |
| 397 | QERE |
| 398 | QEREG |
| 399 | QQRE |
| 400 | QQREW |
| 401 | QQREL |
| 402 | QQREF |
| 403 | KGRE |
| 404 | KGREG |
| 405 | KDRE |
| 406 | KDREV |
| 407 | DECKL |
| 408 | INVCEL |
| 409 | IGVEW |
| 410 | EPEW |
| 411 | GLER |
| 412 | DQEW |
| 413 | DLEW |
| 414 | GIEW |
| 415 | ELEW |
| 416 | GPEW |
| 417 | EWLP |
| 418 | GPER |
| 419 | EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPY |
| | TVRKYNYWGQGTQVTVSSGGCGGHHHHHH |
| 420 | EVQLVESGGGLVQAGGSLRLSCAAS |
| 421 | GSTFSDYG |
| 422 | VGWFRQAPGKGREFVAD |
| 423 | IDWNGEHT |
| 424 | SYADSVKGRFATSRDNAKNTAYLQMNSLKPEDTAVYYC |
| 425 | AADALPYTVRKYNY |
| 426 | WGQGTQVTVSSGGCGGHHHHHH |
| 427 | SYGMH |
| 428 | NYYDGSKKYYADSVKG |
| 429 | ELTGDAFDI |
| 430 | RASQSIGSSLH |
| 431 | VASQSFS |
| 432 | HQSSTLPYT |
| 433 | QVQLVESGGGVVQPGRSLRLSCAASGFTFSSYGMHWVRQAPGKGLEWVAIYYDGSKKYYADSVKGRFTISKDNSKNTLYLQMNSLRAEDTAVYYCARELTGDA |
| | FDIWGQGTKVTVSS |
| 434 | EIVITQSPDFQSVTPKEKVTITCRASQSIGSSLHWYQQKPDQSPKLLIKYASQSFSGVPSKFIGSGSGTDFTLTINSLEAEDAATFYCHQSSTLPYTFGQGTKLEIK |
| 435 | ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT |
| | CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPA |
| | PIEKTISKAKGQPREPQVYTLPPSREEMTKNQVSLTCLVKGFYPSDIAVEWESNGQPENNYKTTPPLVDSDGSFFLYSKLTVDKSRWQQGNVFSCSVMHEALHNH |
| | YTQKSLSLSPG |
| 436 | RTVAAPSVFIFPPSDEQLKSGTASVVCLINNFYPREAKVQWKVDNALQSGNSQESVTEQDSKQSTYSLSSTLTLSKADYEKHKVYACEVTHQGLSSPVTKSFNRGEC |
| 437 | QVQLVESGGGVVQPGRSLRLSCAASGFTFSSYGMHWVRQAPGKGLEWVAIIYYDGSKKYYADSVKGRFTISRDNSKNTLYLQMNSLRAEDTAVYYCARELTGDA |
| | FDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRTPEVTCVVVDVSHEDPEVKENWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGN |
| | VFSCSVMHEALHNHYTQKSLSLSPG |
| 438 | EIVLTQSPDFQSVTPKEKVTITCRASQSIGSSLHWYQQKPDQSPKLLIKYASQSFSGVPSKFIGSGSGTDFTLTINSLEAEDAATFYCHQSSTLPYTFGQGTKLEIKRTV |
| | AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC |
| 439 | CAGGTGCAGCTGGTGGAGTCGGGGGGAGGCGTGGTCCAGCCTGGGAGGTCCCTGAGACTCTCCTGTGCAGCGTCTGGÅTTCACCTTCAGTAGCTATGGCA |
| | TGCACTGGGTCCGCCAGGCTCCAGGCAAGGGGCTGGAGTGGGTGGCAATTATATACTATGATGGAAGTAAAAAATACTATGCAGACTCCGTGAAGGGCC |
| | GATTCACCATCTCCAGAGACAATTCCAAGAACACGTTGTATCTGCAAATGAACAGCCTGAGAGCCGAGGACACGGCTGTGTATTACTGTGCGAGAGAGCTG |
| | ACTGGGGATGCTTTTGATATCTGGGGCCAAGGGACAAAGGTCACCGTCTCTTCA |
| 440 | GAAATTGTGCTGACTCAGTCTCCAGÅCTTTCAGTCTGTGACTCCAAAGGAGAAAGTCACCATCACCTGCCGGGCCAGTCAGAGCATTGGTAGTAGCTTACA |
| | CTGGTACCAGCAGAAACCAGATCAGTCTCCAAAGCTCCTCATCAAGTATGCTTCCCAGTCCTTCTCAGGGGTCCCCTCGAAGTTCATTGGCAGTGGATCTGG |
| | GACAGATTTCACCCTCACCATCAATAGCCTGGAAGCTGAAGATGCTGCAACGTTTTACTGTCATCAGAGTAGTACTTTACCGTACACTTTTGGCCAGGGGAC |
| | CAAGCTGGAGATCAAA |
| 441 | CAGGTGCAGCTGGTGGAGTCGGGGGGAGGCGTGGTCCAGCCTGGGAGGTCCCTGAGACTCTCCTGTGCAGCGTCTGGATTCACCTTCAGTAGCTATGGCA |
| | TGCACTGGGTCCGCCAGGCTCCAGGCAAGGGGCTGGAGTGGGTGGCAATTATATACTATGATGGAAGTAAAAAATACTATGCAGACTCCGTGAAGGGCC |
| | GATTCACCATCTCCAGAGACAATTCCAAGAACACGTTGTATCTGCAAATGAACAGCCTGAGAGCCGAGGACACGGCTGTGTATTACTGTGCGAGAGAGCTG |
| | ACTGGGGATGCTTTTGATATCTGGGGCCAAGGGACAAAGGTCACCGTCTCTTCAGCTAGCACTAAAGGGCCTTCTGTATTTCCCTTGGCCCCGTCCAGCAAA |
| | TCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGTG |
| | TGCACACGTTTCCTGCCGTCCTGCAATCATCGGGCTTGTATAGCCTCAGCTCTGTGGTCACTGTCCCAAGTTCATCGCTGGGCACTCAGACGTATATTTGCAA |
| | TGTGAACCACAAACCTTCAAATACAAAAGTGGATAAACGCGTAGAACCGAAATCGTGTGATAAAACTCACACATGCCCGCCATGCCCGGCACCTGAACTGC |
| | TTGGTGGTCCCAGCGTGTTCCTGTTCCCGCCGAAGCCTAAAGATACTCTAATGATCAGCCGTACGCCAGAGGTGACATGTGTCGTGGTTGACGTGTCCCAC |
| | GAAGATCCCGAAGTTAAGTTCAATTGGTATGTTGATGGTGTAGAGGTACACAATGCTAAGACTAAACCTCGCGAGGAGCAGTACAATTCGACCTATCGTGT |
| | CGTGAGCGTTCTGACCGTCCTTCACCAAGATTGGCTTAACGGCAAAGAATATAAGTGCAAGGTAAGCAATAAAGCACTTCCGGCCCCAATCGAGAAAACCA |
| | TTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTA |
| | AAGGTTTTTATCCTAGCGATATTGCTGTTGAATGGGAATCGAACGGTCAGCCGGAGAATAATTATAAAACAACGCCACCCGTCCTGGATAGCGACGGCTCA |
| | TTTTTTCTGTATAGCAAACTGACTGTAGATAAATCACGGTGGCAGCAGGGCAATGTATTCAGTTGCTCCGTTATGCATGAAGCGTTACATAATCACTACACG |
| | CAGAAATCTCTTAGTCTTTCACCCGGT |
| 442 | GAAATTGTGCTGACTCAGTCTCCAGACTTTCAGTCTGTGACTCCAAAGGAGAAAGTCACCATCACCTGCCGGGCCAGTCAGAGCATTGGTAGTAGCTTACA |
| | CTGGTACCAGCAGAAACCAGATCAGTCTCCAAAGCTCCTCATCAAGTATGCTTCCCAGTCCTTCTCAGGGGTCCCCTCGAAGTTCATTGGCAGTGGATCTGG |
| | GACAGATTTCACCCTCACCATCAATAGCCTGGAAGCTGAAGATGCTGCAACGTTTTACTGTCATCAGAGTAGTACTTTACCGTACACTTTTGGCCAGGGGAC |
| | CAAGCTGGAGATCAAACGTACGGTAGCTGCCCCTTCAGTTTTTATCTTTCCGCCGTCTGACGAGCAGTTAAAATCCGGGACCGCTTCTGTAGTTTGCCTGCT |
| | GAATAATTTTTATCCGCGTGAGGCTAAAGTACAATGGAAAGTCGACAATGCTTTGCAGTCGGGAAATTCACAGGAAAGTGTTACGGAGCAGGATTCTAAA |
| | GATTCCACATATTCACTCAGCTCCACCCTTACACTGAGCAAAGCCGACTATGAAAAACATAAAGTTTACGCATGTGAGGTGACGCACCAAGGATTATCCAGT |
| | CCGGTCACAAAATCGTTTAACCGCGGTGAGTGT |
| 443 | NYGMH |
| 444 | VIYYDGSKNYYEDSVKG |
| 445 | RASQRIGSSLH |
| 446 | HQSSSFPYT |
| 447 | QVQLVESGGDVVQPGRSLRISCAASGHFRNYGMHWVRQAPGKGLEWVAVIYYDGSKNYYEDSVKGRFTISRDNSKNTLYLQMNSIRVEDTAVYYCARELTGD |
| | AFDIWGQGTMVTVSS |
| 448 | ETVLTQSPDFQSLTPKEKVTITCRASQRIGSSLHWYQQKPDQSPKLLIKYASQSFSGVPSRFSGSGSGTDFTLTINSIDAEDAATYYCHQSSSFPYTFGQGTKLQIK |
| 449 | VIYYDGNKKYYVDSVKD |
| 450 | HQSSTFPYT |
| 451 | QVQLVESGGGVVQPGRSLRLSCAASGFTFSSYGMHWVRQAPGKGLEWVAVIYYDGNKKYYVDSVKDRITISRDNSKNTLYLQMNSLRAEDTAVYYCARELTGDA] |
| | FDIWGQGTMVTVSS |
| 452 | EIVLTQSPDFQSVTPKEKVTITCRASQRIGSSLHWYQQKSDQSPKLLIKYASQSFSGVPSRFSGSGSGTDFTLTINSLEAEDAATYYCHQSSTFPYTFGQGTKLEIK |
| 453 | VIVYDGNNKYYADSVKG |
| 454 | ELTGDAFDL |
| 455 | RASQSIGSHUH |
| 456 | HQSSRFPYT |
| 457 | QVQLVESGGGVVQPGRSLRLSCAASGFTFSSYGMHWVRQTPGKGLEWVAVIYYDGNNKYYADSVKGRFTISRENSKNTLYLQMNSLRADDTALYYCARELTGD |
| | AFDLWGQGTMVTVSS |
| 458 | EIVLTQSPDFQSATPKEKVTITCRASQSIGSHLHWYQQKPDQSPKLLIKYASQSFSGVPSRFSGSGSGTDFTLTINRLEDEDAATYYCHQSSRFPYTFGQGTKLDIK |
| 459 | GFX1FX2X3YG; X1 is T or 1, X2 is S or R, X3 is S or N |
| 460 | YYDGX4X5X6; X4 is N or S, X5 is K or N, X6 is K or N |
| 461 | 46TARELTGDAFDX7; X7 is F or L |
| 462 | QX8IGSX9; X8 is S or R, X9 is S or H |
| 463 | SANDWTVDHPQTLFAWEGACIRIPCKYKTPLPKARLDNILLFQNYEFDKATKKFTGTVLYNATKTEKDPESELYLSKQGRVTFLGNRIDNCTLKIHPIRANDSGNIGL |
| | RMTAGTERWMEPIHLNVSEKPFQPYIQMPSEIRESQSVTLTCGLNFSCFGYDILLKWFLEDSEITSITSSVTSITSSVTSSIKNVYTESKLTFQPKWTDHGKSVKCQVQ |
| | HSSKVLSERTVRLDVKYTPKLEIKVNPTEVEKNNSVTMTCRVNSSNPKLRTVAVSWFKDGRPLEDQELEQEQQMSKLILHSVTKDMRGKYRCQASNDIGPGESEE |
| | VELTVHYAPEPSRVHIYPSPAEEGQSVELICESLASPSATNYTWYHNRKPIPGDTQEKLRIPKVSPWHAGNYSCLAENRIGHGKIDQEAKLDVHYAPKAVTTVIQSFT |
| | PILEGDSVTLVCRYNSSNPDVTSYRWNPQGSGSVLKPGVLRIQKVTWDSMPVSCAACNHKCSWALPVILNVHYAPRDVKVLKVSPASEIRAGQRVLLQCDFAESN |
| | PAEVRFFWKKNGSLVQEGRYLSFGSVSPEDSGNYNCMVNNSIGETLSQAWNLQVLYAPRRLRVSISPGDHVMEGKKATLSCESDANPPISQYTWFDSSGQDLHS |
| | SGQK |
| 464 | HQSSX10EPYT; X10 is T, R, or S |
| 465 | CAGGTGCAGCTGGTGGAATCTGGGGGAGACGTGGTCCAGCCTGGGAGGTCCCTGAGACTCTCCTGTGCAGCGTCTGGATTCATCTTCAGGAACTATGGCA |
| | TGCACTGGGTCCGCCAGGCTCCAGGCAAGGGGCTGGAGTGGGTGGCAGTCATTTACTATGATGGAAGTAAAAATTACTATGAAGACTCCGTGAAGGGCCG |
| | ATTCACCATCTCCAGAGACAATTCCAAAAACACGCTGTATCTGCAAATGAACAGCCTGAGAGTCGAGGACACGGCTGTGTATTACTGTGCGAGAGAACTTA |
| | CTGGGGATGCTTTTGATATCTGGGGCCAAGGGACAATGGTCACCGTCTCTTCA |
| 466 | GAAATTGTGCTGACTCAGTCTCCAGACTTTCAGTCTCTGACTCCAAAGGAGAAAGTCACCATCACCTGTCGGGCCAGTCAGAGAATTGGTAGTAGCTTACAC |
| | TGGTACCAGCAGAAACCAGATCAGTCTCCAAAACTCCTCATCAAGTATGCTTCCCAGTCCTTCTCAGGGGTCCCCTCGAGGTTCAGTGGCAGTGGATCTGGG |
| | ACAGATTTCACCCTCACCATCAATAGCCTGGATGCTGAAGATGCTGCAACGTATTACTGTCATCAGAGTAGTAGTTTCCCGTACACTTTTGGCCAGGGGACC |
| | AAGCTGCAGATCAAA |
| 467 | CAGGTGCAGCTGGTGGAGTCTGGGGGAGGCGTGGTCCAGCCTGGGAGGTCCCTGAGACTCTCCTGTGCAGCGTCTGGATTCACCTTCAGTAGCTATGGCA |
| | TGCACTGGGTCCGCCAGGCTCCAGGCAAGGGGCTGGAGTGGGTGGCGGTTATATACTATGATGGAAATAAAAAATACTATGTTGACTCCGTGAAGGACCG |
| | AATCACCATCTCCAGAGACAATTCCAAGAACACGTTGTATCTGCAAATGAACAGCCTGAGAGCCGAGGACACGGCTGTGTATTATTGTGCGAGAGAACTAA |
| | CTGGGGATGCTTTTGATATCTGGGGCCAAGGGACAATGGTCACCGTCTCTTCA |
| 468 | GAAATTGTGCTGACTCAGTCTCCAGACTTTCAGTCTGTGACTCCAAAGGAGAAAGTCACCATCACCTGCCGGGCCAGTCAGAGAATTGGAAGTAGCTTACA |
| | CTGGTACCAGCAGAAATCAGATCAGTCTCCAAAGCTCCTCATCAAGTATGCTTCCCAGTCCTTCTCAGGGGTCCCCTCGAGGTTCAGTGGCAGTGGATCTGG |
| | GACAGATTTCACCCTCACCATCAATAGCCTGGAAGCTGAAGATGCTGCAACGTATTACTGTCATCAGAGTAGTACGTTCCCGTACACTTTTGGCCAGGGGAC |
| | CAAGCTAGAGATCAAA |
| 469 | CAGGTGCAGCTGGTGGAGTCTGGGGGGGGCGTGGTCCAGCCTGGGAGGTCCCTGAGACTCTCCTGTGCAGCGTCTGGATTCACCTTCAGTAGCTATGGCA |
| | TGCACTGGGTCCGCCAGACTCCAGGCAAGGGGCTGGAGTGGGTGGCAGTTATATACTATGATGGAAATAATAAATACTATGCAGACTCCGTGAAGGGCCG |
| | ATTCACCATCTCCAGAGAGAATTCCAAGAACACGCTGTATCTGCAAATGAACAGCCTGAGAGCCGATGACACGGCTCTATATTACTGTGCGAGAGAACTAA |
| | CTGGGGATGCTTTTGATCTCTGGGGCCAAGGGACAATGGTCACCGTCTCTTCA |
| 470 | GAAATTGTGCTGACTCAGTCTCCAGATTTTCAGTCTGCGACTCCAAAGGAGAAAGTCACCATCACCTGCCGGGCCAGTCAGAGCATTGGTAGTCACTTACAC |
| | TGGTATCAGCAGAAACCAGATCAGTCTCCAAAGCTCCTCATCAAGTATGCTTCCCAGTCCTTCTCAGGGGTCCCCTCGAGGTTCAGTGGCAGTGGATCTGGG |
| | ACAGATTTCACCCTCACCATCAATAGACTGGAAGATGAAGATGCTGCAACGTATTACTGTCATCAGAGTAGTAGGTTCCCGTACACTTTTGGCCAGGGGACC |
| | AAGCTGGATATCAAA |
| 471 | DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESK |
| | TEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSN |
| | DTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQ |
| | ILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSC |
| | NYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEK |
| | NGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEP |
| | VKVQHSGAYWCQGTNSVGKGRSPLSTLTVYYSPETIGRR |
| 472 | DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESK |
| | TEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSN |
| | DTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQ |
| | ILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSC |
| | NYNSSNPSVTRYEWKPHGAWEEPSLGVLKIQNVGWQNTTIACAACNSWCSWASPVALNVQYAPRDVRVRKIKPLSEIHSGNSVSLQCDFSSSHPKEVQFFWEK |
| | NGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEP |
| | VKVQHSGAYWCQGTNSVGKGRSPLSTLTVYYSPETIGRRGGGGSGLNDIFEAQKIEWHEGGGGSHHHHHH |
| 473 | QYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMP |
| | IREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHP |
| | KEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPY |
| | HSQKLRLEPVKVQHSGAYWCQGTNSVGKGRSPLSTLTVYYSPETIGRRGGGGSGLNDIFEAQKIEWHEGGGGSHHHHHH |
| 474 | DSSKWVFEHPETLYAWEGACVWIPCTYRALDGDLESFILFHNPEYNKNTSKFDGTRLYESTKDGKVPSEQKRVQFLGDKNKNCTLSIHPVHINDSGQLGERMESK |
| | TEKWMERIHLNVSERPFPPHIQLPPEIQESQEVTLTCLLNFSCYGYPIQLQWLLEGVPMRQAAVTSTSLTIKSVFTRSELKESPQWSHHGKIVTCQLQDADGKFLSN |
| | DTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAGGGGSG |
| | LNDIFEAQKIEWHEGGGGSHHHHHH |
| 475 | GYYIH |
| 476 | WINPNSGGTNYAQKFQG |
| 477 | EDTWNYIDY |
| 478 | RASQSISSHIN |
| 479 | AASSLHS |
| 480 | QQSYSTPRT |
| 481 | QVQLVQSGAEVKKPGASVKVSCKASGYTFTGYVIHWVRQAPGQGLEWMGWINPNSGGTNYAQKFQGRVTMTRDTSIGTAYMELSRLRSDDTAIYYCTREDTW |
| | NYIDYWGQGTLVTVSS |
| 482 | DIQMTQSPSSLSASVGDRVTITCRASQSISSHINWYQQKPGKAPKLLIYAASSLHSGVPSRFSGSGSGTDFTLTISSLQPEDFATYCCQQSYSTPRTFGQGTKVEIK |
| 483 | QVQLVQSGAEVKKPGASVKVSCKASGYTFTGYYIHWVRQAPGQGLEWMGWINPNSGGTNYAQKFQGRVTMTRDTSIGTAYMELSRLRSDDTAIYYCTREDTW |
| | NYIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSN |
| | TKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQ |
| | GNVFSCSVMHEALHNHYTQKSLSLSPG |
| 484 | DIQTTQSPSSLSASVGDRVTITCRASQSISSHINWYQQKPGKAPKILIYAASSLHSGVPSRFSGSGSGTDFTLTISSLQPEDFATYCCQQSYSTPRTFGQGTKVEIKRT |
| | IVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC |
| 485 | CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACTATA |
| | TACACTGGGTGCGACAGGCCCCTGGACAAGGGCTTGAGTGGATGGGATGGATCAACCCTAACAGTGGTGGCACAAACTATGCACAGAAGTTTCAGGGCA |
| | GGGTCACCATGACCAGGGACACGTCCATCGGCACAGCCTACATGGAGCTGAGCAGGCTGAGATCTGACGACACGGCCATTTATTACTGTACGAGAGAGGA |
| | CACCTGGAACTACATTGACTACTGGGGCCAGGGAACCCTGGTCACCGTCTCCTCA |
| 486 | GACATCCAGATGACCCAGTCTCCATCCTCCCTGTCTGCATCTGTAGGAGACAGAGTCACCATCACTTGCCGGGCAAGTCAGAGCATTAGCAGCCATTTAAAT |
| | TGGTATCAGCAGAAACCAGGGAAAGCCCCTAAACTCCTGATCTATGCTGCATCCAGTTTACACAGTGGGGTCCCATCAAGGTTCAGTGGCAGTGGATCTGG |
| | GACAGATTTCACTCTCACCATCAGCAGTCTGCAACCTGAAGATTTTGCAACTTACTGCTGTCAACAGAGTTACAGTACCCCTCGGACGTTCGGCCAGGGGAC |
| | CAAGGTGGAAATCAAA |
| 487 | CAGGTGCAGCTGGTGCAGTCTGGGGCTGAGGTGAAGAAGCCTGGGGCCTCAGTGAAGGTCTCCTGCAAGGCTTCTGGATACACCTTCACCGGCTACT?T? |
| | TACACTGGGTGCGACAGGCCCCTGGACAAGGGCTTGAGTGGATGGGATGGATCAACCCTAACAGTGGTGGCACAAACTATGCACAGAAGTTTCAGGGCA |
| | GGGTCACCATGACCAGGGACACGTCCATCGGCACAGCCTACATGGAGCTGAGCAGGCTGAGATCTGACGACACGGCCATTTATTACTGTACGAGAGAGGA |
| | CACCTGGAACTACATTGACTACTGGGGCCAGGGAACCCTGGTCACCGTCTCCTCAGCTAGCACTAAAGGGCCTTCTGTATTTCCCTTGGCCCCGTCCAGCAA |
| | ATCGACCTCGGGAGGGACAGCCGCCCTGGGTTGCCTTGTGAAAGATTATTTCCCTGAGCCAGTTACCGTAAGTTGGAACAGTGGGGCGCTGACAAGTGGT |
| | GTGCACACGTTTCCTGCCGTCCTGCAATCATCGGGCTTGTATAGCCTCAGCTCTGTGGTCACTGTCCCAAGTTCATCGCTGGGCACTCAGACGTATATTTGCA |
| | ATGTGAACCACAAACCTTCAAATACAAAAGTGGATAAACGCGTAGAACCGAAATCGTGTGATAAAACTCACACATGCCCGCCATGCCCGGCACCTGAACTG |
| | CTTGGTGGTCCCAGCGTGTTCCTGTTCCCGCCGAAGCCTAAAGATACTCTAATGATCAGCCGTACGCCAGAGGTGACATGTGTCGTGGTTGACGTGTCQCAC |
| | GAAGATCCCGAAGTTAAGTTCAATTGGTATGTTGATGGTGTAGAGGTACACAATGCTAAGACTAAACCTCGCGAGGAGCAGTACAATTCGACCTATCGTGT |
| | CGTGAGCGTTCTGACCGTCCTTCACCAAGATTGGCTTAACGGCAAAGAATATAAGTGCAAGGTAAGCAATAAAGCACTTCCGGCCCCAATCGAGAAAACCA |
| | TTTCCAAGGCCAAAGGTCAACCAAGAGAACCCCAGGTGTATACTCTTCCGCCTTCTCGTGAGGAAATGACTAAAAATCAAGTATCCCTTACGTGTCTGGTTA |
| | AAGGTTTTTATCCTAGCGATATTGCTGTTGAATGGGAATCGAACGGTCAGCCGGAGAATAATTATAAAACAACGCCACCCGTCCTGGATAGCGACGGCTCA |
| | TTTTTTCTGTATAGCAAACTGACTGTAGATAAATCACGGTGGCAGCAGGGCAATGTATTCAGTTGCTCCGTTATGCATGAAGCGTTACATAATCACTACACG |
| | CAGAAATCTCTTAGTCTTTCACCCGGT |
| 488 | GACATCCAGATGACCCAGTCTCCATCCTCCCTGTCTGCATCTGTAGGAGACAGAGTCACCATCACTTGCCGGGCAAGTCAGAGCATTAGCAGCCATTTAAAT |
| | TGGTATCAGCAGAAACCAGGGAAAGCCCCTAAACTCCTGATCTATGCTGCATCCAGTTTACACAGTGGGGTCCCATCAAGGTTCAGTGGCAGTGGATCTGG |
| | GACAGATTTCACTCTCACCATCAGCAGTCTGCAACCTGAAGATTTTGCAACTTACTGCTGTCAACAGAGTTACAGTACCCCTCGGACGTTCGGCCAGGGGAC |
| | CAAGGTGGAAATCAAACGTACGGTAGCTGCCCCTTCAGTTTTTATCTTTCCGCCGTCTGACGAGCAGTTAAAATCCGGGACCGCTTCTGTAGTTTGCCTGCT |
| | GAATAATTTTTATCCGCGTGAGGCTAAAGTACAATGGAAAGTCGACAATGCTTTGCAGTCGGGAAATTCACAGGAAAGTGTTACGGAGCAGGATTCTAAA |
| | GATTCCACATATTCACTCAGCTCCACCCTTACACTGAGCAAAGCCGACTATGAAAAACATAAAGTTTACGCATGTGAGGTGACGCACCAAGGATTATCCAGT |
| | CCGGTCACAAAATCGTTTAACCGCGGTGAGTGT |
| 489 | TSGMCVS |
| 490 | LIDWDGDNYYSTSLKT |
| 491 | MGYTYGYDAFDI |
| 492 | RASQSIGSLLH |
| 493 | FASQSLS |
| 494 | HQSRSLPFT |
| 495 | QVTLRESGPALVKPTQTLTLTCTFSGISLTTSGMCVSWIRQPPGKALEWLALIDWDGDNYYSTSLKTRLTISKDTSKNQVVLTMASMDPVDTATYYCARMGYTYG |
| | YDAFDIWGQGTMVTVSS |
| 496 | EIVLTQSPEFQSVTPTEKVTITCRASQSIGSLLHWYQQKPDQSPKLLIKFASQSLSGVPSRFSGSGSGTDFTLTINSLEAEDAATYYCHQSRSLPFTFGPGTKVDIN |
| 497 | QVTLRESGPALVKPTQTLTLTCTFSGISLTTSGMCVSWIRQPPGKALEWLALIDWDGDNYYSTSLKTRLTISKDTSKNQVVLTMASMDPVDTATVYCARMGYTYG |
| | YDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPS |
| | NTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQD |
| | WINGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQ |
| | QGNVFSCSVMHEALHNHYTQKSLSLSPG |
| 498 | EIVITQSPEFQSVTPTEKVTITCRASQSIGSLLHWYQQKPDQSPKLLIKFASQSLSGVPSRFSGSGSGTDFTLTINSLEAEDAATYYCHQSRSLPFTFGPGTKVDINRTV |
| | AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC |
| 499 | CAGGTCACCTTGAGGGAGTCTGGTCCTGCGCTGGTGAAACCCACACAGACCCTCACACTGACCTGCACCTTCTCTGGGATCTCACTTACCACTAGTGGAATG |
| | TGTGTGAGTTGGATCCGTCAGCCCCCAGGGAAGGCCCTGGAGTGGCTTGCACTCATTGATTGGGATGGTGATAACTACTACAGCACATCTCTGAAGACCAG |
| | GCTCACCATCTCCAAGGACACCTCCAAAAACCAGGTGGTCCTTACAATGGCCAGCATGGACCCTGTGGACACAGCCACGTATTACTGTGCACGGATGGGAT |
| | ACACCTATGGTTACGATGCTTTTGATATCTGGGGCCAAGGGACAATGGTCACCGTCTCTTCA |
| 500 | GAAATTGTGCTGACTCAGTCTCCAGAATTTCAGTCTGTGACTCCAACGGAGAAAGTCACCATCACCTGCCGGGCCAGTCAGAGCATTGGTAGTCCTTACAC |
| | TGGTACCAGCAGAAACCAGATCAGTCTCCAAAGCTCCTCATCAAGTTTGCTTCCCAGTCCCTCTCAGGGGTCCCCTCGAGGTTCAGTGGCAGTGGATCTGGG |
| | ACAGATTTCACCCTCACCATCAATAGCCTGGAAGCTGAAGATGCTGCAACGTATTACTGTCATCAGAGTAGGAGTTTACCATTCACTTTCGGCCCTGGGACC |
| | AAAGTGGATATCAAT |
| 501 | TCAGGTCACCTTGAGGGAGTCTGGTCCTGCGCTGGTGAAACCCACACAGACCCTCACACTGACCTGCACCTTCTCTGGGATCTCACTTACCACTAGTGGAATG |
| | TGTGTGAGTTGGATCCGTCAGCCCCCAGGGAAGGCCCTGGAGTGGCTTGCACTCATTGATTGGGATGGTGATAACTACTACAGCACATCTCTGAAGACCAG |
| | GCTCACCATCTCCAAGGACACCTCCAAAAACCAGGTGGTCCTTACAATGGCCAGCATGGACCCTGTGGACACAGCCACGTATTACTGTGCACGGATGGGAT |
| | ACACCTATGGTTACGATGCTTTTGATATCTGGGGCCAAGGGACAATGGTCACCGTCTCTTCAGCTAGCACTAAAGGGCCTTCTGTATTTCCCTTGGCCCCGTC |
| | CAGCAAATCGACCTCGGGAGGGACAGCCGCCCTGGGTTGCCTTGTGAAAGATTATTTCCCTGAGCCAGTTACCGTAAGTTGGAACAGTGGGGCGCTGACA |
| | AGTGGTGTGCACACGTTTCCTGCCGTCCTGCAATCATCGGGCTTGTATAGCCTCAGCTCTGTGGTCACTGTCCCAAGTTCATCGCTGGGCACTCAGACGTAT |
| | ATTTGCAATGTGAACCACAAACCTTCAAATACAAAAGTGGATAAACGCGTAGAACCGAAATCGTGTGATAAAACTCACACATGCCCGCCATGCCCGGCACC |
| | TGAACTGCTTGGTGGTCCCAGCGTGTTCCTGTTCCCGCCGAAGCCTAAAGATACTCTAATGATCAGCCGTACGCCAGAGGTGACATGTGTCGTGGTTGACG |
| | TGTCCCACGAAGATCCCGAAGTTAAGTTCAATTGGTATGTTGATGGTGTAGAGGTACACAATGCTAAGACTAAACCTCGCGAGGAGCAGTACAATTCGACC |
| | TATCGTGTCGTGAGCGTTCTGACCGTCCTTCACCAAGATTGGCTTAACGGCAAAGAATATAAGTGCAAGGTAAGCAATAAAGCACTTCCGGCCCCAATCGA |
| | GAAAACCATTICCAAGGCCAAAGGTCAACCAAGAGAACCCCAGGTGTATACTCTTCCGCCTTCTCGTGAGGAAATGACTAAAAATCAAGTATCCCTTACGTG |
| | TCTGGTTAAAGGTTTTTATCCTAGCGATATTGCTGTTGAATGGGAATCGAACGGTCAGCCGGAGAATAATTATAAAACAACGCCACCCGTCCTGGATAGCG |
| | ACGGCTCATTTTTTCTGTATAGCAAACTGACTGTAGATAAATCACGGTGGCAGCAGGGCAATGTATTCAGTTGCTCCGTTATGCATGAAGCGTTACATAATC |
| | ACTACACGCAGAAATCTCTTAGTCTTTCACCCGGT |
| 502 | GAAATTGTGCTGACTCAGTCTCCAGAATTTCAGTCTGTGACTCCAACGGAGAAAGTCACCATCACCTGCCGGGCCAGTCAGAGCATTGGTAGTCTCTTACAC |
| | TGGTACCAGCAGAAACCAGATCAGTCTCCAAAGCTCCTCATCAAGTTTGCTTCCCAGTCCCTCTCAGGGGTCCCCTCGAGGTTCAGTGGCAGTGGATCTGGG |
| | ACAGATTTCACCCTCACCATCAATAGCCTGGAAGCTGAAGATGCTGCAACGTATTACTGTCATCAGAGTAGGAGTTTACCATTCACTTTCGGCCCTGGGACC |
| | AAAGTGGATATCAATCGTACGGTAGCTGCCCCTTCAGTTTTTATCTTTCCGCCGTCTGACGAGCAGTTAAAATCCGGGACCGCTTCTGTAGTTTGCCTGCTGA |
| | ATAATTITTATCCGCGTGAGGCTAAAGTACAATGGAAAGTCGACAATGCTTTGCAGTCGGGAAATTCACAGGAAAGTGTTACGGAGCAGGATTCTAAAGAT |
| | TCCACATATTCACTCAGCTCCACCCTTACACTGAGCAAAGCCGACTATGAAAAACATAAAGTTTACGCATGTGAGGTGACGCACCAAGGATTATCCAGTCCG |
| | GTCACAAAATCGTTTAACCGCGGTGAGTGT |
| 503 | TSGMCVS |
| 504 | LIDWDDDKYYSTSLKT |
| 505 | TRYIYGYDYFDY |
| 506 | RASQRISSQLN |
| 507 | AASSLQS |
| 508 | QQTYSKPIT |
| 509 | QVTLRESGPALVKTTQTLTLTCTESGFSLSTSGMCVSWIRQPPGKALEWIALIDWDDDKYYSTSLKTRLTISKDTSKSQVVLTMTSMDPVDTATYYCARIRYIYGYDY |
| | IFDYWGQGTLVTVSS |
| 510 | DIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIK |
| 511 | QVTLRESGPALVKTTQTLTLTCTFSGFSLSTSGMCVSWIRQPPGKALEWIALIDWDDDKYYSTSLKTRLTISKDTSKSQVVLTMTSMIDPVDTATYYCARIRYIYGYDY |
| | IFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTK |
| | VDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLN |
| | GKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGN |
| | VFSCSVMHEALHNHYTQKSLSLSPG |
| 512 | DIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIKRTV |
| | AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSENRGEC |
| 513 | CAGGTCACCTTGAGGGAGTCTGGTCCTGCGCTGGTGAAAACCACACAGACCCTCACACTGACCTGCACCTTCTCTGGGTTCTCACCAGCACTAGTGGAATG |
| | TGTGTGAGCTGGATCCGTCAGCCCCCAGGGAAGGCCCTGGAGTGGCTTGCACTCATTGATTGGGATGATGATAAATACTACAGCACATCTCTGAAGACCAG |
| | GCTCACCATCTCCAAGGACACCTCCAAATCCCAGGTGGTCCTTACAATGACCAGCATGGACCCTGTGGACACAGCCACGTATTACTGTGCACGGATACGATA |
| | CATCTATGGTTACGACTACTTTGACTACTGGGGCCAGGGAACCCTGGTCACCGTCTCCTCA |
| 514 | EIVLTQTPSSLSASLGDRVTISCRASQSIGSSLHWYQQKPDQSPKLLIKYASQSFSGVPSKFIGSGSGTDFTLTINSLEAEDAATFYCHQSSTLPYTFGQGTKLEIK |
| 515 | MALPVTALLLPLALLLHAARP |
| 516 | GGACAGATCGCCTGGAGACGCCATCCACGCTGTTTTGACCTCCATAGAAGACACCGGGACCGATCCAGCCTCCGCGGCCGGGAACGGTGCATTGGAACGC |
| | GGATTCCCCGTGCCAAGAGTGACTCACCGTCCTTGACACG |
| 517 | CGGGTGGCATCCCTGTGACCCCTCCCCAGTGCCTCTCCTGGCCCTGGAAGTTGCCACTCCAGTGCCCACCAGCCTTGTCCTAATAAAATTAAGTTGCATC |
| 518 | GGGTCAAGATTAGAGAACGGTCGTAGCATTATCGGAGGTTCTGCCACC |
| 519 | GGCACATTTGAATAAATTCTATTACCAGTTCAAGCTTGGCGTAATCATGGTCATAGCTG |
| 520 | MDMRVPAQLLGLLLLWLPGAKC |
| 521 | LSNSIMYFSHFVPVFLPAKPTTTPAPRPPTPAPTIASQPLSLRPEACRPAAGGAVHTRGLDFACDIVIWAPLAGTCGVLLLSLVITLYCNHRN |
| 522 | IEVMYPPPYLDNERSNGTIIHVKGKHLCPSPLFPGPSKPFWVLVVVGGVLACYSLLVTVAHIFWV |
| 523 | QVQLVESGGGVVQPGRSLRISCAASGFTFSSYGMHWVRQA |
| | PGKGLEWVAIIVYDGSKKYYADSVKGRFTISRDNSKNTLYLQMNSLRAEDTAVYYCAREL |
| | TGDAFDIWGQGTKVTVSS |
| 524 | EIVLTQTPSSLSASLGDRVTIS |
| | CRASQSIGSSLHWYQQKPDQSPKLLIKYASQSFSGVPSKFIGSGSGTDFTLTINSLEAEDAATFYCHQSSTLPYTFGQGTKLEIK |
| | AATFYCHQSSTLPYTFGQGTKLEIK |
| 525 | IQVQLVQSGAEVKKPGASVKVSCKASGYTFTGYYIHWVRQA |
| | PGQGLEWMGWINPNSGGTNYAQKFQGRVTMTRDTSIGTAYMELSRLRSDDTAIYYCTRED |
| | TWNYIDYWGQGTLVTVSS |
| 526 | DIQMTQSPSSLSASVGDRVTIT |
| | CRASQSISSHLNWYQQKPGKAPKLLIYAASSLHSGVPSRFSGSGSGTDFTLTISSLQPEDFATYCCQQSYSTPRTFGQGTKVEIK |
| | FATYCCQQSYSTPRTFGQGTKVEIK |
| 527 | QVTLRESGPALVKPTQTLTLTCTFSGISLTTSGMCVSWIRQPPGKALEWLALIDWDGDNYYSTSLKTRLTISKDTSKNQVVLTMASMIDPVDTATVYCARMGYTYG |
| | YDAFDIWGQGTMVTVSS |
| 528 | EIVLTQTPSSLSASLGDRVTISCRASQSIGSLLHWYQQKPDQSPKLLIKFASQSLSGVPSRFSGSGSGTDFTLTINSLEAEDAATYYCHQSRSLPFTFGPGTKVDIN |
| 529 | QVTLRESGPALVKTTQTLTLTCTFSGFSLSTSGMCVSWIR |
| | QPPGKALEWLALIDWDDDKYYSTSLKTRLTISKDTSKSQVVLTMTSMDPVDTATYYCARI |
| | RYIYGYDYFDYWGQGTLVTVSS |
| 530 | DIQMTQSPSSLSASVGDRVTITCRASQRISSQLNWYQQKPGKAPKLLIYAASSLQSGVPSRFSGSGSGTEFTLTISSLQPEDIATYCCQQTYSKPITFGQGTRLEIK |
| 531 | QVQLVQSGAEVKKPGASVKVSCKASGYTFTGYVAIWVRQAPGQGLEWMGWVDPKNGDTNYAHKFQGRVTMIWDTSISTAYMELSRLISDDTAVYYCARDA |
| | DFDYWGQGTLVTVSS |
| 532 | DVVMTQTPSSLSASLGDRVTISCRASQSLVYIDGNTYLNWFRQRPGQSPRRLIYKVSNRDYGVPDRESGSGSGTDFTLKISRVEAEDVGVYYCMQGTHWPLTFGG |
| | GTKVEIK |
| 533 | DVVMTQSPLSLPVTLGQPASISCRSSQSLVYIDGNTYLNWFRQRPGQSPRRLIYKVSNRDYGVPDRESGSGSGTDFTLKISRVEAEDVGVYYCMQGTHWPLTFGG |
| | GTKVEIK |
| 534 | EIVLTQSPDFQSVTPKEKVTITCRASQSIGSSLHWYQQKPDQSPKLLIKYASQSFSGVPSKFIGSGSGTDFTLTINSLEAEDAATFYCHQSSTLPYTFGQGTKLEIK |
| 535 | EIVLTQSPEFQSVTPTEKVTITCRASQSIGSLLHWYQQKPDQSPKLLIKFASQSLSGVPSRFSGSGSGTDFTLTINSLEAEDAATYYCHQSRSLPFTFGPGTKVDIN |
| 536 | QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTG |
| | DLEDAFDIWGQGTMVTVSS |
| 537 | DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK |
| 538 | TTTPAPRPPTPAPTIASQPLSLRPEACRPAAGGAVHTRGLDFACDIYIWAPLAGTCGVLLLSLVITLYCNHRN |
| 539 | SLRPEACRPAAGGAVHTRGLDFACDIYIWAPLAGTCGVLLLSLVITLYCNHRN |
| 540 | LSNSIMYFSHFVPVFLPAKPTTTPAPRPPTPAPTIASQPLSLRPEACRPAAGGAVHTRGLDFACDIYIWAPLAGTCGVLLLSLVITLYCNHRN |
| 541 | IEVMYPPPYLDNEKSNGTIIHVKGKHLCPSPLFPGPSKPF |
| 542 | WVLVVVGGVLACYSLLVTVAFIIFWV |
| 543 | RSKRSRLLHSDYMNMTPR4RPGPTRKHYQPYAPPRDFAAYRS |
| 544 | RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYD |
| | ALHMQALPPR |
| 545 | MDMRVPAQLLGLLLLWLPGAKCQVQLVESGGGVVQPGRSLRLSCAASGFTFSSYGMHWVR |
| | QAPGKGLEWVAHIYYDGSKKYYADSVKGRFTISRDNSKNTLYLQMNSLRAEDTAVYYCAR |
| | ELTGDAFDIWGQGTKVTVSSGGGGSGGGGSGGGGSGGGGSEIVLTQTPSSLSASLGDRVT |
| | ISCRASQSIGSSLHWYQQKPDQSPKLLIKYASQSFSGVPSKFIGSGSGTDFTLTINSLEA |
| | EDAATFYCHQSSTLPYTFGQGTKLEIKAAAIEVMYPPPYLDNEKSNGTIIHVKGKHLCPS |
| | PLFPGPSKPFWVLVVVGGVLACYSLLVTVAFIIFWVRSKRSRLLHSDYMNMTPRRPGPTR |
| | KHYQPYAPPRDFAAYRSRVKFSRSADAPAYQQGQNQLYNELNLGRREEYDVLDKRRGRDP |
| | EMGGKPRRKNPQEGLYNELQKDKMAEAYSEIGMKGERRRGKGHDGLYQGLSTATKDTYDA |
| 546 | MDMRVPAQLLGLLLLWLPGAKCQVQLVESGGGVVQPGRSLRLSCAASGFTFSSYGMHWVR |
| | QAPGKGLEWVAIIYYDGSKKYYADSVKGRFTISRDNSKNTLYLQMNSLRAEDTAVYYCAR |
| | ELTGDAFDIWGQGTKVTVSSGGGGSGGGGSGGGGSGGGGSEIVLTQTPSSLSASLGDRVT |
| | ISCRASQSIGSSLHWYQQKPDQSPKLLIKYASQSFSGVPSKFIGSGSGTDFTLTINSLEA |
| | EDAATFYCHQSSTLPYTFGQGTKLEIKASSLRPEACRPAAGGAVHTRGLDFACDIYIWAP |
| | LAGTCGVLLLSLVITLYCNHRNVRSKRSRLLHSDYMNMTPRRPGPTRKHYQPYAPPRDFA |
| | AYRSRVKESRSADAPAYQQGQNQLYNELNLGRREEYDVLDKRRGRDPEMGGKPRRKNPQE |
| | GLYNELQKDKMAEAYSEIGMKGERRRGKGHDGLYQGLSTATKDTYDALHMQALPPR |
| 547 | MDMRVPAQLLGLLLLWLPGAKCQVQLVESGGGVVQPGRSLRLSCAASGFTFSSYGMHWVR |
| | QAPGKGLEWVAHIYYDGSKKYYADSVKGRFTISRDNSKNTLYLQMNSLRAEDTAVYYCAR |
| | ELTGDAFDIWGQGTKVTVSSGGGGSGGGGSGGGGSGGGGSEIVLTQTPSSLSASLGDRVT |
| | ISCRASQSIGSSLHWYQQKPDQSPKLLIKYASQSFSGVPSKFIGSGSGTDFTLTINSLEA |
| | EDAATFYCHQSSTLPYTFGQGTKLEIKASTTTPAPRPPTPAPTIASQPLSLRPEACRPAA |
| | GGAVHTRGLDFACDIYIWAPLAGTCGVLLLSLVITLYCNHRNVRSKRSRLLHSDYMNMTP |
| | RRPGPTRKHYQPYAPPRDFAAYRSRVKFSRSADAPAYQQGQNQLYNELNLGRREEYDVLD |
| | KRRGRDPEMGGKPRRKNPQEGLYNELQKDKMAEAYSEIGMKGERRRGKGHDGLYQGLSTA TKDTDALHMQALPPR |
| 548 | MDMRVPAQLLGLLLLWLPGAKCQVQLVESGGGVVQPGRSLRLSCAASGFTFSSYGMHWVR |
| | QAPGKGLEWVAHIYYDGSKKYYADSVKGRATISRDNSKNTLYLQMNSLRAEDTAVYYCAR |
| | ELTGDAFDIWGQGTKVTVSSGGGGSGGGGSGGGGGGGGSEIVLTQTPSSLSASLGDRVT |
| | ISCRASQSIGSSLHWYQQKPDQSPKLLIKYASQSFSGVPSKFIGSGSGTDFTLTINSLEA |
| | EDAATFYCHQSSTLPYTFGQGTKLEIKASLSNSIMYFSHFVPVFLPAKPTTTPAPRPPTP |
| | APTIASQPLSLRPEACRPAAGGAVHTRGLDFACDIYIWAPLAGTCGVLLLSLVITLYCNH |
| | RNVRSKRSRLLHSDYMNMTPRRPGPTRKHYQPYAPPRDFAAYRSRVKFSRSADAPAYQQG |
| | QNQLYNELNLGRREEYDVLDKRRGRDPEMGGKPRRKNPQEGLYNELQKDKMAEAYSEIGM KGERRRGKGHDGLYQGLSTATKDTYDALHMQALP |
| 549 | MDMRVPAQLLGLLLLWLPGAKCQVQLVQSGAEVKKPGASVKVSCKASGYTFTGYYIHWVR |
| | QAPGQGLEWMGWINPNSGGTNYAQKFQGRVTMTRDTSIGTAYMELSRLRSDDTAIYYCTR |
| | EDTWNYIDYWGQGTLVTVSSGGGGSGGGGSGGGGSGGGGSDIQMTQSPSSLSASVGDRVT |
| | ITCRASQSISSHLNWYQQKPGKAPKLLIYAASSLHSGVPSRFSGSGSGTDFTLTISSLQP |
| | EDFATYCCQQSYSTPRTFGQGTKVEIKAAAIEVMYPPPYLDNEKSNGTIIHVKGKHLCPS |
| | PLFPGPSKPFWVLVVVGGVLACYSLLVTVAFIIFWVRSKRSRLLHSDYMNMTPRRPGPTR |
| | KHYQPYAPPRDFAAYRSRVKFSRSADAPAYQQGQNQLYNELNLGRREEYDVLDKRRGRDP |
| | EMGGKPRRKNPQEGLYNELQKDKMAEAYSEIGMKGERRRGKGHDGLYQGLSTATKDTYDA LHMQALPPR |
| 550 | MDMRVPAQLLGLLLLWLPGAKCQVQLVQSGAEVKKPGASVKVSCKASGYTFTGYYIHWVR |
| | QAPGQGLEWMGWINPNSGGTNYAQKFQGRVTMTRDTSIGTAYMELSRLRSDDTAIYYCTR |
| | EDTWNYIDYWGQGTLVTVSSGGGGGGGGSGGGGSGGGGSDIQMTQSPSSLSASVGDRVT |
| | ITCRASQSISSHINWYQQKPGKAPKLLIYAASSLHSGVPSRFSGSGSGTDFTLTISSLQP |
| | EDFATYCCQQSYSTPRTFGQGTKVEIKASSLRPEACRPAAGGAVHTRGLDFACDIVIWAP |
| | LAGTCGVLLLSLVITLYCNHRNVRSKRSRLLHSDYMNMTPRRPGPTRKHYQPYAPPRDFA |
| | AYRSRVKESRSADAPAYQQGQNQLYNELNLGRREEYDVLDKRRGRDPEMGGKPRRKNPQE |
| | GLYNELQKDKMAEAYSEIGMKGERRRGKGHDGLYQGLSTATKDTYDALHMQALPPR |
| 551 | MDMRVPAQLIGLLLLWLPGAKCQVQLVQSGAEVKKPGASVKVSCKASGYTFTGYYIHWVR |
| | QAPGQGLEWMGWINPNSGGTNYAQKFQGRVTMTRDTSIGTAYMELSRLRSDDTAIYYCTR |
| | EDTWNYIDYWGQGTLVTVSSGGGGSGGGGSGGGGSGGGGSDIQMTQSPSSLSASVGDRVT |
| | ITCRASQSISSHLNWYQQKPGKAPKLLIYAASSLHSGVPSRFSGSGSGTDFTLTISSLQP |
| | EDFATYCCQQSYSTPRTFGQGTKVEIKASTTTPAPRPPTPAPTIASQPLSLRPEACRPAA |
| | GGAVHTRGLDFACDIYIWAPLAGTCGVLLLSLVITLYCNHRNVRSKRSRLLHSDYMNMTP |
| | RRPGPTRKHYQPYAPPRDFAAYRSRVKESRSADAPAYQQGQNQLYNELNLGRREEYDVLD |
| | KRRGRDPEMGGKPRRKNPQEGLYNELQKDKMAEAYSEIGMKGERRRGKGHDGLYQGLSTA TKDTYDALHMQALPPR |
| 552 | MDMRVPAQLLGLLLLWLPGAKCQVQLVQSGAEVKKPGASVKVSCKASGYTFTGYYIHWVR |
| | QAPGQGLEWMGWINPNSGGTNYAQKFQGRVTMTRDTSIGTAYMELSRLRSDDTAIYYCTR |
| | EDTWNYIDYWGQGTLVTVSSGGGGSGGGGSGGGGSGGGGSDIQMTQSPSSLSASVGDRVT |
| | ITCRASQSISSHINWYQQKPGKAPKLLIYAASSLHSGVPSRFSGSGSGTDFTLTISSLQP |
| | EDFATYCCQQSYSTPRTFGQGTKVEIKASTTTSIMYFSHFVPVFLPAKPTTTPAPRPPTP |
| | APTIASQPLSLRPEACRPAAGGAVHTRGLDFACDIYIWAPLAGTCGVLLLSLVITLYCNH |
| | RNVRSKRSRLLHSDYMNMTPRRPGPTRKHYQPYAPPRDFAAYRSRVKFSRSADAPAYQQG |
| | QNQLYNELNLGRREEYDVLDKRRGRDPEMGGKPRRKNPQEGLYNELQKDKMAEAYSEIGM KGERRRGKGHDGLYQGLSTATKDTYDALHMQALPPR |
| 553 | MDMRVPAQLIGLLLLWIPGAKCQVTLRESGPALVKPTQTLTLTCTFSGISLTTSGMCVSW |
| | HRQPPGKALEWLALIDWDGDNYYSTSLKTRLTISKDTSKNQVVLTMASMDPVDTATYYCA |
| | RMGYTYGYDAFDIWGQGTMVTVSSGGGGSGGGGSGGGGSGGGGSEIVLTQTPSSLSASLG |
| | DRVTISCRASQSIGSLLHWYQQKPDQSPKLLIKFASQSLSGVPSRFSGSGSGTDFTLTIN |
| | SLEAEDAATYYCHQSRSLPFTFGPGTKVDINAAAIEVMYPPPYLDNEKSNGTHIHVKGKH |
| | LCPSPLFPGPSKPFWVLVVVGGVLACYSLLVTVAFIIFWVRSKRSRLLHSDYMNMTPRRP |
| | GPTRKHYQPYAPPRDFAAYRSRVKFSRSADAPAYQQGQNQLYNELNLGRREEYDVLDKRR |
| | GRDPEMGGKPRRKNPQEGLYNELQKDKMAEAYSEIGMKGERRRGKGHDGLYQGLSTATKD TYDALHMQALPPR |
| 554 | MDMRVPAQLLGLLLLWLPGAKCQVTLRESGPALVKPTQTLTLTCTESGISLTTSGMCVSW |
| | IRQPPGKALEWLALIDWDGDNYYSTSLKTRLTISKDTSKNQVVLTMASMDPVDTATYYCA |
| | RMGYTYGYDAFDIWGQGTMVTVSSGGGGSGGGGSGGGGSGGGGSEIVLTQTPSSLSASLG |
| | DRVTISCRASQSIGSLLHWYQQKPDQSPKLLIKFASQSLSGVPSRESGSGSGTDETLTIN |
| | SLEAEDAATYYCHQSRSLPFTFGPGTKVDINASSLRPEACRPAAGGAVHTRGLDFACDIY |
| | IWAPLAGTCGVLLLSLVITLYCNHRNVRSKRSRLLHSDYMNMTPRRPGPTRKHYQPYAPP |
| | RDFAAYRSRVKFSRSADAPAYQQGQNQLYNELNLGRREEYDVLDKRRGRDPEMGGKPRRK |
| | NPQEGLYNELQKDKMAEAYSEIGMKGERRRGKGHDGLYQGLSTATKDTYDALHMQALPPR |
| 555 | MDMRVPAQLLGLLLLWLPGAKCQVTLRESGPALVKPTQTLTLTCTESGISLTTSGMCVSW |
| | IRQPPGKALEWLALIDWDGDNYYSTSLKTRLTISKDTSKNQVVLTMASMDPVDTATYYCA |
| | RMGYTYGYDAFDIWGQGTMVTVSSGGGGSGGGGSGGGGSGGGGSEIVLTQTPSSLSASLG |
| | DRVTISCRASQSIGSLLHWYQQKPDQSPKLLIKFASQSLSGVPSRFSGSGSGTDFTLTIN |
| | SLEAEDAATYYCHQSRSLPFTFGPGTKVDINASTTTPAPRPPTPAPTIASQPLSLRPEAC |
| | RPAAGGAVHTRGLDFACDIYIWAPLAGTCGVLLLSLVITLYCNHRNVRSKRSRLLHSDYM |
| | NMTPRRPGPTRKHYQPYAPPRDFAAYRSRVKFSRSADAPAYQQGQNQLYNELNLGRREEY |
| | DVLDKRRGRDPEMGGKPRRKNPQEGLYNELQKDKMAEAYSEIGMKGERRRGKGHDGLYQG LSTATKDTYDALHMQALPPR |
| 556 | MDMRVPAQLLGLLLLWLPGAKCQVTLRESGPALVKPTQTLTLTCTESGISLTTSGMCVSW |
| | IRQPPGKALEWLALIDWDGDNYYSTSLKTRLTISKDTSKNQVVLTMASMDPVDTATYYCA |
| | RMGYTYGYDAFDIWGQGTMVTVSSGGGGSGGGGSGGGGSGGGGSEIVLTQTPSSLSASLG |
| | DRVTISCRASQSIGSLLHWYQQKPDQSPKLLIKFASQSLSGVPSRFSGSGSGTDFTLTIN |
| | SLEAEDAATYYCHQSRSLPFTFGPGTKVDINASLSNSIMYFSHFVPVFLPAKPTTTPAPR |
| | PPTPAPTIASQPLSLRPEACRPAAGGAVHTRGLDFACDIYIWAPLAGTCGVLLLSLVITI |
| | YCNHRNVRSKRSRLLHSDYMNMTPRRPGPTRKHYQPYAPPRDFAAYRSRVKFSRSADAPA |
| | YQQGQNQLYNELNLGRREEYDVLDKRRGRDPEMGGKPRRKNPQEGLYNELQKDKMAEAYS EIGMKGERRRGKGHDGLYQGLSTATKDTYDALHMQALPPR |
| 557 | MDMRVPAQLLGLLLLWLPGAKCQVTLRESGPALVKTTQTLTLTCTFSGFSLSTSGMCVSW |
| | HRQPPGKALEWLALIDWDDDKYYSTSLKTRLTISKDTSKSQVVLTMTSMDPVDTATYYCA |
| | RIRYIYGYDYFDYWGQGTLVTVSSGGGGSGGGGSGGGGSGGGGSDIQMTQSPSSLSASVG |
| | DRVTITCRASQRISSQLNWYQQKPGKAPKLLIVAASSLQSGVPSRESGSGSGTEFTLTIS |
| | SLQPEDIATYCCQQTYSKPITFGQGTRLEIKAAAIEVMYPPPYLDNEKSNGTHIHVKGKH |
| | LCPSPLFPGPSKPFWVLVVVGGVLACYSLLVTVAFIIFWVRSKRSRLLHSDYMNMTPRRP |
| | GPTRKHYQPYAPPRDFAAYRSRVKFSRSADAPAYQQGQNQLYNELNLGRREEYDVLDKAR |
| | GRDPEMGGKPRRKNPQEGLYNELQKDKMAEAYSEIGMKGERRRGKGHDGLYQGLSTATKD TYDALHMQALPPR |
| 558 | MDMRVPAQLIGLLLLWLPGAKCQVTLRESGPALVKTTQTLTLTCTFSGFSLSTSGMCVSW |
| | IRQPPGKALEWLALIDWDDDKYYSTSLKTRLTISKDTSKSQVVLTMTSMDPVDTATYYCA |
| | RIRYIYGYDYFDYWGQGTLVTVSSGGGGSGGGGSGGGGSGGGGSDIQMTQSPSSLSASVG |
| | DRVTITCRASQRISSQLNWYQQKPGKAPKLLIYAASSLQSGVPSRFSGSGSGTEFTLTIS |
| | SLQPEDIATYCCQQTYSKPITFGQGTRLEIKASSLRPEACRPAAGGAVHTRGLDFACDIY |
| | IWAPLAGTCGVLLLSLVITLYCNHRNVRSKRSRLLHSDYMNMTPRRPGPTRKHYQPYAPP |
| | RDFAAYRSRVKFSRSADAPAYQQGQNQLYNELNLGRREEYDVLDKRRGRDPEMGGKPRRK |
| | NPQEGLYNELQKDKMAEAYSEIGMKGERRRGKGHDGLYQGLSTATKDTYDALHMQALPPR |
| 559 | MDMRVPAQLLGLLLLWLPGAKCQVTLRESGPALVKTTQTLTLTCTFSGFSLSTSGMCVSW |
| | HRQPPGKALEWLALIDWDDDKYYSTSLKTRLTISKDTSKSQVVLTMTSMDPVDTATYYCA |
| | RIRYIYGYDYFDYWGQGTLVTVSSGGGGSGGGGSGGGGSGGGGSDIQMTQSPSSLSASVG |
| | DRVTITCRASQRISSQLNWYQQKPGKAPKLLIYAASSLQSGVPSRFSGSGSGTEFTLTIS |
| | SLQPEDIATYCCQQTYSKPITFGQGTRLEIKASTTTPAPRPPTPAPTIASQPLSLRPEAC |
| | RPAAGGAVHTRGLDFACDIYIWAPLAGTCGVLLLSLVITLYCNHRNVRSKRSRLLHSDYM |
| | NMTPRRPGPTRKHYQPYAPPRDFAAYRSRVKFSRSADAPAYQQGQNQLYNELNLGRREEY |
| | DVLDKRRGRDPEMGGKPRRKNPQEGLYNELQKDKMAEAYSEIGMKGERRRGKGHDGLYQG LSTATKDTYDALHMQALPPR |
| 560 | MDMRVPAQLLGLLLLWLPGAKCQVTLRESGPALVKTTQTLTLTCTFSGFSLSTSGMCVSW |
| | HRQPPGKALEWLALIDWDDDKYYSTSLKTRLTISKDTSKSQVVLTMTSMDPVDTATYYCA |
| | RIRYIYGYDYFDYWGQGTLVTVSSGGGGSGGGGSGGGGSGGGGSDIQMTQSPSSLSASVG |
| | DRVTITCRASQRISSQLNWYQQKPGKAPKLLIVAASSLQSGVPSRFSGSGSGTEFTLTIS |
| | SLQPEDIATYCCQQTYSKPITFGQGTRLEIKASLSNSIMYFSHFVPVFLPAKPTTTPAPR |
| | PPTPAPTIASQPLSLRPEACRPAAGGAVHTRGLDFACDIYIWAPLAGTCGVLLLSLVITL |
| | YCNHRNVRSKRSRLLHSDYMNMTPRRPGPTRKHYQPYAPPRDFAAYRSRVKFSRSADAPA |
| | YQQGQNQLYNELNLGRREEYDVLDKRRGRDPEMGGKPRRKNPQEGLYNELQKDKMAEAYS EIGMKGERRRGKGHDGLYQGLSTATKDTYDALHMQALPPR |
| 561 | MDMRVPAQLIGLLLLWLPGAKCQVQLQQSGPGLVKPSQTLSLTCAISGDSVSSNSAAWNW |
| | HRQSPSRGLEWLGRTYYRSKWYNDYAVSVKSRITINPDTSKNQFSLQLNSVTPEDTAVYY |
| | ICAREVTGDLEDAFDIWGQGTMVTVSSGGGGSGGGGGGGGSGGGGSDIQMTQSPSSLSAS |
| | VGDRVTITCRASQTIWSYLNWYQQRPGKAPNLLIYAASSLQSGVPSRFSGRGSGTDFTLT |
| | ISSLQAEDFATYYCQQSYSIPQTFGQGTKLEIKAAAIEVMYPPPYLDNEKSNGTHIHVKG |
| | KHLCPSPLFPGPSKPFWVLVVVGGVLACYSLLVTVAFIIFWVRSKRSRLLHSDYMNMTPR |
| | RPGPTRKHYQPYAPPRDFAAYRSRVKFSRSADAPAYQQGQNQLYNELNLGRREEYDVLDK |
| | RRGRDPEMGGKPRRKNPQEGLYNELQKDKMAEAYSEIGMKGERRRGKGHDGLYQGLSTAT KDTYDALHMQALPPR |
| 562 | MDMRVPAQLLGLLLLWLPGAKCQVQLQQSGPGLVKPSQTLSLTCAISGDSVSSNSAAWNW |
| | IRQSPSRGLEWLGRTYYRSKWYNDYAVSVKSRITINPDTSKNQFSLQLNSVTPEDTAVYY |
| | CAREVTGDLEDAFDIWGQGTMVTVSSGGGGSGGGGSGGGGSGGGGSDIQMTQSPSSLSAS |
| | VGDRVTITCRASQTIWSYLNWYQQRPGKAPNLLIYAASSLQSGVPSRFSGRGSGTDFTLT |
| | ISSLQAEDFATYYCQQSYSIPQTFGQGTKLEIKASSLRPEACRPAAGGAVHTRGLDFACD |
| | IYIWAPLAGTCGVLLLSLVITLYCNHRNVRSKRSRLLHSDYMNMTPRRPGPTRKHYQPYA |
| | PPRDFAAYRSRVKFSRSADAPAYQQGQNQLYNELNLGRREEYDVLDKRRGRDPEMGGKPR |
| | RKNPQEGLYNELQKDKMAEAYSEIGMKGERRRGKGHDGLYQGLSTATKDTYDALHMQALP |
| 563 | MDMRVPAQLLGLLLLWLPGAKCQVQLQQSGPGLVKPSQTLSLTCAISGDSVSSNSAAWNW |
| | IRQSPSRGLEWLGRTYYRSKWYNDYAVSVKSRITINPDTSKNQFSLQLNSVTPEDTAVYY |
| | CAREVTGDLEDAFDIWGQGTMVTVSSGGGGSGGGGSGGGGSGGGGSDIQMTQSPSSLSAS |
| | VGDRVTITCRASQTIWSYLNWYQQRPGKAPNLLIYAASSLQSGVPSRESGRGSGTDFTLT |
| | ISSLQAEDFATYYCQQSYSIPQTFGQGTKLEIKASTTTPAPRPPTPAPTIASQPLSLRPE |
| | ACRPAAGGAVHTRGLDFACDIYIWAPLAGTCGVLLLSLVITLYCNHRNVRSKRSRLLHSD |
| | YMINMTPRRPGPTRKHYQPYAPPRDFAAYRSRVKFSRSADAPAYQQGQNQLYNELNLGRRE |
| | EYDVLDKRRGRDPEMGGKPRRKNPQEGLYNELQKDKMAEAYSEIGMKGERRRGKGHDGLY QGLSTATKDTYDALHMQALPPR |
| 564 | MDMRVPAQLLGLLLLWLPGAKCQVQLQQSGPGLVKPSQTLSLTCAISGDSVSSNSAAWNW |
| | HRQSPSRGLEWLGRTYYRSKWYNDYAVSVKSRITINPDTSKNQFSLQLNSVTPEDTAVYY |
| | CAREVTGDLEDAFDIWGQGTMVTVSSGGGGSGGGGSGGGGSGGGGSDIQMTQSPSSLSAS |
| | VGDRVTITCRASQTIWYSYLNWYQQRPGKAPNLLIYAASSLQSGVPSRFSGRGSGTDFTLT |
| | ISSLQAEDFATYYCQQSYSIPQTFGQGTKLEIKASLSNSIMYFSHFVPVFLPAKPTTTPA |
| | PRPPTPAPTIASQPLSLRPEACRPAAGGAVHTRGLDFACDIYIWAPLAGTCGVLLLSLVI |
| | TLYCNHRNVRSKRSRLLHSDYMNMTPRRPGPTRKHYQPYAPPRDFAAYRSRVKFSRSADA |
| | PAYQQGQNQLYNELNLGRREEYDVLDKRRGRDPEMGGKPRRKNPQEGLYNELQKDKMAEA |
| | YSEIGMKGERRRGKGHDGLYQGLSTATKDTYDALHMQALPPR |
| 565 | malpvtalll plalllhaar psqfrvspid rtwnigetve lkcqvlisnp tsgcswlfqp |
| | rgaaasptfl lylsqnkpka aegidtqrfs gkrigdtfvl tlsdfrrene gyyfcsalsn |
| | simyfshfvp vflpakpttt paprpptpap tiasqplsir peacrpaagg avhtrgldfa |
| | cdiyiwapla gtcgvlllsl vitlycnhrn rrrvckcprp vvksgdkpsi saryv |
| 566 | GFTFRSFGMS |
| 567 | SISGSGSDTL |
| 568 | GGSLSR |
| 569 | QVQLVESGGGIVQPGGSIRISCAASGFTLDYYAIGWFRQAPGKEREGVSCISSSDGSTYYADSVKGRATISRDNAKNTVYLQMNSLKPEDTAVYYCAA |
| 570 | MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRR |
| | ENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCK |
| | CPRPVVKSGDKPSLSARYV |
| 571 | GFTFX₁DYAIG, wherein X₁ is selected from D and E |
| 572 | CIRTYDX₂X₃TY, wherein X₂ is selected from G and E; wherein X₃ is selected from N and Q |
| 573 | GSYYACAX₄YSRPDPSEX₅HVDX₆DY, wherein X₄ is selected from K, E and Y; wherein X₅ is selected from N and G; and/or wherein X₆ is selected from |
| | M and L. |
| 574 | CIRTYDX₁X₂TYYX₃DSVKG, wherein X₁ is selected from G and E; wherein X₂ is selected from N and Q; wherein X₃ is selected from I and A; |

### ENUMERATED EMBODIMENTS

The following enumerated embodiments are representative of some aspects of the invention.

1. A lipid nanoparticle (LNP) comprising:
(a) a lipid-immune cell targeting group conjugate comprising the compound of Formula (II): [Lipid]-[optional linker]-[antibody],
(b) an ionizable cationic lipid, and
(c) a nucleic acid, wherein the nucleic acid is encapsulated in the LNP; wherein
   (i) the antibody is an immunoglobulin single variable domain (ISVD) that specifically binds to human CD8alpha, comprising complementarity-determining regions 1 (CDR1), 2 (CDR2), and 3 (CDR3) of an ISVD having the sequence selected from the group consisting of SEQ ID NOs: 160 to 179, or
   (ii) the antibody is an ISVD that specifically binds to human CD8alpha, and the nucleic acid encodes a polypeptide comprising CD22-chimeric antigen receptor (CAR), or both (i) and (ii).

2. The LNP of embodiment 1, wherein the ISVD specifically binding to CD8alpha comprises CDR1, CDR2, and CDR3 according to the Abm CDR definition,
wherein CDR1 is chosen from the group consisting of:
   (i) SEQ ID NO: 244; and
   (ii) amino acid sequences that have 3, 2, or 1 amino acid difference with at least one of the amino acid sequences of SEQ ID NO: 244;
wherein CDR2 is chosen from the group consisting of:
   (i) SEQ ID NO: 246; and
   (ii) amino acid sequences that have 3, 2, or 1 amino acid difference with at least one of the amino acid sequences of SEQ ID NO: 246; and
wherein CDR3 is chosen from the group consisting of:
   (i) SEQ ID NO: 248; and
   (ii) amino acid sequences that have 3, 2, or 1 amino acid difference with at least one of the amino acid sequences of SEQ ID NO: 248.

3. The LNP of embodiment 1 or embodiment 2, wherein the antibody specifically binding to human CD8alpha is covalently coupled to the Lipid in Formula (II) via a linker comprising polyethylene glycol (PEG).

4. The LNP of embodiment 3, wherein the Lipid in Formula (II) covalently coupled to the antibody is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

5. The LNP of embodiment 4, wherein the Lipid in Formula (II) covalently coupled to the antibody is DSPE.

The LNP of any one of embodiments 3 to 5, wherein the PEG is PEG 3400 (PEG 3.4K).

7. The LNP of any one of embodiments 1 to 6, wherein the immunoglobulin single variable domain comprises SEQ ID NO: 9, SEQ ID NO: 169, or SEQ ID NO: 44.

8. The LNP of any one of embodiments 1 to 7, wherein the LNP further comprises a structural lipid, a neutral phospholipid, or a free PEG-lipid, or any combination thereof.

9. The LNP of embodiment 8, wherein the structural lipid comprises or is sterol.

10. The LNP of embodiment 8 or embodiment 9, wherein the sterol comprises or is cholesterol.

11. The LNP of any one of embodiments 8 to 10, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.

12. The LNP of any one of embodiments 8 to 11, wherein the neutral phospholipid comprises or is DSPC.

13. The LNP of any one of embodiments 8 to 12, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols.

14. The LNP of any one of embodiments 8 to 13, wherein the free PEG lipid is PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]—N,N-ditetradecylacetamide, diacylphosphatidylethanolamine comprising Dipalmitoyl (C16) chain or Distearoyl (C18) chain, or a PEG-distearoyl-phosphatidylethanolamine (PEG-DSPE) lipid.

15. The LNP of embodiment 14, wherein the PEG-DAG comprises PEG-DMG, PEG-DPG, or PEG-DSG, or any combination thereof.

16. The LNP of embodiment 15, wherein the free PEG-lipid comprises PEG-DPG.

17. The LNP of embodiment 16, wherein the PEG-DPG comprises or is PEG 2000-DPG (DPG-PEG 2000).

18. The LNP of any one of embodiments 1 to 17, wherein the nucleic acid comprises or is RNA.

19. The LNP of embodiment 18, wherein the RNA comprises or is mRNA.

20. The LNP of embodiment 19, wherein the mRNA encodes a synthetic T cell receptor (synTCR) or a Chimeric Antigen Receptor (CAR).

21. The LNP of embodiment 19 or 20, wherein the mRNA comprises a 5′ Cap, a 5′ untranslated region (UTR), a sequence encoding a polypeptide, a 3′ UTR, and optionally a polyA tail

22. The LNP of any one of embodiments 1 to 21, wherein the nucleic acid comprises:
(1) optionally, a 5′ cap;
(2) optionally, a 5′ UTR region;
(3) optionally, nucleotides encoding a Lead peptide sequence;
(4) nucleotides encoding an antibody heavy chain variable region (VH);
(5) optionally, nucleotides encoding a Linker A;
(6) nucleotides encoding an antibody light chain variable region (VL);
(7) nucleotides encoding a Linker B;
(8) nucleotides encoding a Hinge domain;
(9) nucleotides encoding a Transmembrane domain;
(10) nucleotides encoding a Co-stimulatory domain;
(11) nucleotides encoding a Signaling domain;
(12) optionally, a 3′ UTR region; and
(13) optionally, a polyA tail.

23. The LNP of embodiment 22, wherein the nucleic acid comprises the following formula, arranged from 5′ to 3′: 5′ UTR (optional)-nucleotides encoding the Lead peptide sequence (optional)-nucleotides encoding the antibody heavy chain variable region (VH)—nucleotides encoding the Linker A (optional)-nucleotides encoding the antibody light chain variable region (VL)-nucleotides encoding Linker B (optional)-nucleotides encoding the Hinge-nucleotides encoding the Transmembrane domain-nucleotides encoding the Co-stimulatory domain-nucleotides encoding the Signaling domain-3′ UTR (optional).

24. The LNP of embodiment 21, wherein the polypeptide encoded by the nucleic acid comprises an antibody specifically binding to B-cell, a Hinge domain and a Transmembrane domain (Hinge and Transmembrane domains), a Co-stimulatory domain, and a Signaling domain.

25. The LNP of embodiment 24, wherein the polypeptide encoded by the nucleic acid comprises the following formula, arranged from N-terminus to C-terminus:

[Lead peptide sequence (optional)]-[antibody specifically binding to B-cell]-[Linker B (optional)]-[Hinge domain]-[Transmembrane domain]-[Co-stimulatory domain]-[Signaling domain].

26. The LNP of embodiment 22, embodiment 23, or embodiment 25, wherein the optional Lead peptide sequence comprises a signal peptide.

27. The LNP of embodiment 26, wherein the signal peptide is derived from CD8 (SEQ ID NO: 565).

28. The LNP of embodiment 26 wherein the signal peptide comprises SEQ ID NO: 515 or SEQ ID NO: 520, or a sequence having at least 80%, at least 85%, at least 90%, at least 95%, at least 96%, at least 97%, at least 98%, or at least 99% or higher identity to SEQ ID NO: 515 or SEQ ID NO: 520.

29. The LNP of any one of embodiments 24 to 28, wherein the antibody specifically binding to B-cell comprises the following formula: [antibody specifically binding to B-cell, heavy chain variable region (VH)]-[Linker A (optional)]-[antibody specifically binding to B-cell, light chain variable region (VL)].

30. The LNP of any one of embodiments 24 to 29, wherein the antibody specifically binding to B-cell is an antibody that specifically binds to human CD22.

31. The LNP of any one of embodiments 24 to 30, wherein the antibody specifically binding to B-cell comprises an anti-CD22 ScFv.

32. The LNP of embodiment 31, wherein the anti-CD22 ScFV comprises a heavy chain variable (VH) domain and an antibody light chain variable (VL) domain, wherein the VH and VL domains comprise:
(1) a complementarity determining region-1 (CDR1), a CDR2, and a CDR3 of a heavy chain variable region (VH) comprising the amino acid sequence of SEQ ID NO: 433 and a CDR1, CDR2, and a CDR3 of a light chain variable region (VL) comprising the amino acid sequence of SEQ ID NO: 434;
(2) a complementarity determining region-1 (CDR1), a CDR2, and a CDR3 of a heavy chain variable region (VH) comprising the amino acid sequence of SEQ ID NO: 447 and a CDR1, CDR2, and a CDR3 of a light chain variable region (VL) comprising the amino acid sequence of SEQ ID NO: 448;
(3) a complementarity determining region-1 (CDR1), a CDR2, and a CDR3 of a heavy chain variable region (VH) comprising the amino acid sequence of SEQ ID NO: 457 and a CDR1, CDR2, and a CDR3 of a light chain variable region (VL) comprising the amino acid sequence of SEQ ID NO: 458;
(4) a complementarity determining region-1 (CDR1), a CDR2, and a CDR3 of a heavy chain variable region (VH) comprising the amino acid sequence of SEQ ID NO: 481 and a CDR1, CDR2, and a CDR3 of a light chain variable region (VL) comprising the amino acid sequence of SEQ ID NO: 482;
(5) a complementarity determining region-1 (CDR1), a CDR2, and a CDR3 of a heavy chain variable region (VH) comprising the amino acid sequence of SEQ ID NO: 495 and a CDR1, CDR2, and a CDR3 of a light chain variable region (VL) comprising the amino acid sequence of SEQ ID NO: 496;
(6) a complementarity determining region-1 (CDR1), a CDR2, and a CDR3 of a heavy chain variable region (VH) comprising the amino acid sequence of SEQ ID NO: 509 and a CDR1, CDR2, and a CDR3 of a light chain variable region (VL) comprising the amino acid sequence of SEQ ID NO: 510;
(7) a complementarity determining region-1 (CDR1), a CDR2, and a CDR3 of a heavy chain variable region (VH) comprising the amino acid sequence of SEQ ID NO: 523 and a CDR1, CDR2, and a CDR3 of a light chain variable region (VL) comprising the amino acid sequence of SEQ ID NO: 524;
(8) a complementarity determining region-1 (CDR1), a CDR2, and a CDR3 of a heavy chain variable region (VH) comprising the amino acid sequence of SEQ ID NO: 525 and a CDR1, CDR2, and a CDR3 of a light chain variable region (VL) comprising the amino acid sequence of SEQ ID NO: 526;
(9) a complementarity determining region-1 (CDR1), a CDR2, and a CDR3 of a heavy chain variable region (VH) comprising the amino acid sequence of SEQ ID NO: 527 and a CDR1, CDR2, and a CDR3 of a light chain variable region (VL) comprising the amino acid sequence of SEQ ID NO: 528;
(10) a complementarity determining region-1 (CDR1), a CDR2, and a CDR3 of a heavy chain variable region (VH) comprising the amino acid sequence of SEQ ID NO: 529 and a CDR1, CDR2, and a CDR3 of a light chain variable region (VL) comprising the amino acid sequence of SEQ ID NO: 530; or (11) a complementarity determining region-1 (CDR1), a CDR2, and a CDR3 of a heavy chain variable region (VH) comprising the amino acid sequence of SEQ ID NO: 531 and a CDR1, CDR2, and a CDR3 of a light chain variable region (VL) comprising the amino acid sequence of SEQ ID NO: 532.

33. The LNP of embodiment 32,
wherein the VH domain of the anti-CD22 ScFV comprises a CDR-H1 sequence comprising the amino acid sequence of SYGMH (SEQ ID NO: 427), a CDR-H2 sequence comprising the amino acid sequence of IIYYDGSKKYYADSVKG (SEQ ID NO: 428), and a CDR-H3 sequence comprising the amino acid sequence of ELTGDAFDI (SEQ ID NO: 429); and
wherein the VL domain of the anti-CD22 ScFV comprises a CDR-L1 sequence comprising the amino acid sequence of RASQSIGSSLH (SEQ ID NO: 430), a CDR-L2 sequence comprising the amino acid sequence of YASQSFS (SEQ ID NO:431), and a CDR-L3 sequence comprising the amino acid sequence of HQSSTLPYT (SEQ ID NO: 432).

34. The LNP of embodiment 33, wherein the anti-CD22 ScFV comprises a VH domain comprising SEQ ID NO: 523, and a VL domain comprising SEQ ID NO: 524.

35. The LNP of embodiment 34, wherein the VH domain and the VL domain is connected through Linker A, wherein the Linker A is selected from the group consisting of SEQ ID Nos 337-348.

36. The LNP of any one of embodiments 22, 23, and 26-35, wherein the Linker A is (GGGGS) 4 (SEQ ID NO: 344).

37. The LNP of any one of embodiments 22, 23, and 25-36, wherein the Linker B is AS or AAA.

38. The LNP of any one of embodiments 22 to 37, wherein the hinge and transmembrane domains are derived from CD8 hinge and transmembrane domains.

39. The LNP of embodiment 38, wherein the hinge and transmembrane domains comprise SEQ ID NO: 538, SEQ ID NO: 539, or SEQ ID NO: 540.

40. The LNP of any one of embodiments 22 to 39, wherein the hinge and transmembrane domains are derived from CD28 hinge and transmembrane domains.

41. The LNP of embodiment 40, wherein the CD28 hinge and transmembrane domains have the amino acid sequence selected from the group consisting of (i) SEQ ID NO: 522; (ii) sequences having at least 80%, at least 85%, at least 90%, at least 95%, at least 96%, at least 97%, at least 98%, or at least 99% or higher identity with SEQ ID NO: 522; and (iii) sequences that have 3, 2, or 1 amino acid difference with SEQ ID NO: 522.

42. The LNP of any one of embodiments 22 to 41, wherein the Co-stimulatory domain is a CD28 Co-stimulatory domain.

43. The LNP of embodiment 42, wherein the CD28 Co-stimulatory domain comprises SEQ ID NO: 543.

44. The LNP of any one of embodiments 22 to 43, wherein the Signaling domain is derived from a CD3z signaling domain.

45. The LNP of any one of embodiments 22 to 44, wherein the Signaling domain has the amino acid sequence selected from the group consisting of (i) sequence of SEQ ID NO: 544; (ii) sequences that have at least 80% sequence identity with SEQ ID NO: 544; and (iii) sequences that have 3, 2, or 1 amino acid difference with SEQ ID NO: 544.

46. The LNP of any one of embodiments 1 to 45, wherein the polypeptide encoded by the nucleic acid comprises or consists of SEQ ID NO: 116, SEQ ID NO: 126, or SEQ ID NO: 127.

47. The LNP of any one of embodiments 1 to 45, wherein the nucleic acid sequence encoding a polypeptide comprises a sequence encoding the polypeptide of SEQ ID NO: 116, SEQ ID NO: 126, or SEQ ID NO: 127.

48. The LNP of embodiment 47, wherein the nucleic acid sequence comprises SEQ ID NO: 108, SEQ ID NO: 124, or SEQ ID NO: 125.

49. The LNP of embodiment 47, wherein the nucleic acid sequence comprises SEQ ID NO: 139 or SEQ ID NO: 147.

50. The LNP of any one of embodiments 1 to 49, wherein the nucleic acid comprises pseudouridine.

51. The LNP of embodiment 50, wherein the pseudouridine is N1-methyl-pseudouridine.

52. The LNP of any one of embodiments 1 to 51, wherein the ionizable cationic lipid comprises a compound of Formula (I):

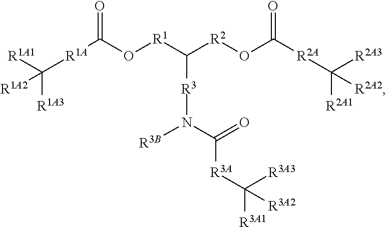

or a salt thereof, or both, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

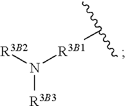

R^{3B1} is C₁₋₆ alkylene; and
R^{3B2} and R^{3B3} are each independently H, unsubstituted C₁₋₆ alkyl, or C₁₋₆ alkyl substituted with 1 or 2 —OH.

53. The LNP of embodiment 52, wherein:
R¹, R², and R³ are each independently a bond or methylene;
R^{1A} and R^{2A} are each C₁₋₁₀ alkylene;
R^{3A} is C₁₋₅ alkylene;
R^{1A1}, R^{1A2}, R^{2A1}, R^{2A2}, R^{3A1}, and R^{3A2} are each H;
R^{1A3} and R^{2A3} are each C₁₋₂₀ alkenyl;
R^{3A3} is-C(O)O(C₁₋₂₀ alkyl);
R^{3B} is

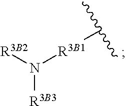

R^{3B1} is C₂₋₄ alkylene; and
R^{3B2} and R^{3B3} are each methyl.

54. The LNP of embodiment 52 or 53, wherein R^{3B1} is —(CH₂)₃—.

55. The LNP of any one of embodiments 1 to 54, wherein the ionizable cationic lipid comprises

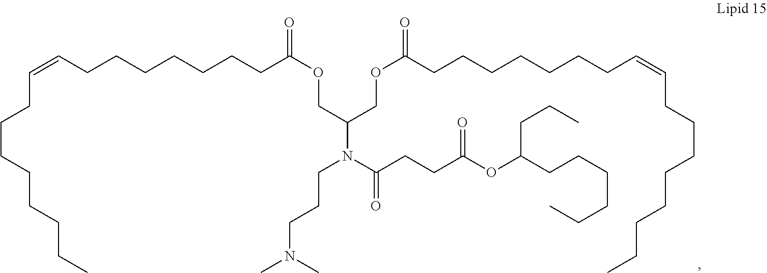 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof, or both.

56. The LNP of any one of embodiments 1 to 55, wherein the cationic lipid has a concentration between about 10 mol % and about 60 mol % of the LNP.

56a. The LNP of embodiment 56, wherein the cationic lipid has a concentration of between about 45 mol % and 55 mol %, such as about 48 mol %, about 49 mol %, or about 50 mol % of the LNP.

56b. The LNP of embodiment 56, wherein the cationic lipid has a concentration of about 49.2 mol % of the LNP.

57. The LNP of any one of embodiments 1 to 56b, wherein the LNP comprises cholesterol at a concentration between about 25 mol % and about 45 mol % of the LNP.

57a. The LNP of embodiment 57, wherein the LNP comprises cholesterol at a concentration of about 29 mol % of the LNP.

57b. The LNP of embodiment 57, wherein the LNP comprises cholesterol at a concentration of about 39 mol % of the LNP.

58. The LNP of any one of embodiments 1 to 57b, wherein the LNP comprises DSPC at a concentration between about 5 mol % and about 25% mol % of the LNP.

58a. The LNP of embodiment 58, wherein the LNP comprises DSPC at a concentration of about 20 mol % of the LNP.

58b. The LNP of embodiment 58, wherein the LNP comprises DSPC at a concentration of about 10 mol % of the LNP.

59. The LNP of any one of embodiments 1 to 58b, wherein the LNP comprises DPG-PEG2K at a concentration between about 0.5 mol % and about 2.5 mol % of the LNP.

59a. The LNP of embodiment 59, wherein the LNP comprises DPG-PEG2K at a concentration of about 1.5 mol % of the LNP.

60. The LNP of any one of embodiments 1 to 59a, wherein the LNP comprises cationic lipid at a concentration between about 49 mol % and about 50 mol % of the LNP, such as about 49.1, 49.2, 49.3, 49.4, 49.5, 49.6, 49.7, 49.8, or 49.9 mol %.

61. The LNP of any one of embodiments 1 to 60, wherein the LNP comprises cholesterol at a concentration between about 25 mol % and about 30 mol % of the LNP, such as about 25, 26, 27, 28, 29, or 30 mol %.

62. The LNP of any one of embodiments 1 to 61, wherein the LNP comprises DSPC at a concentration between about 9 mol % and about 21 mol % of the LNP.

63. The LNP of any one of embodiments 1 to 62, wherein the LNP comprises DPG-PEG2K at a concentration between about 1.4 mol % and about 1.6 mol % of the LNP.

64. The LNP of any one of embodiments 1 to 55, wherein the LNP comprises cationic lipid at a concentration between about 10 and about 20 g per gram of mRNA in the LNP.

65. The LNP of any one of embodiments 1 to 64, wherein the LNP comprises cholesterol at a concentration between about 3.0 and about 5.0 g per gram of mRNA in the LNP.

66. The LNP of any one of embodiments 1 to 65, wherein the LNP comprises DSPC at a concentration between about 2.0 and about 5.0 g per gram of mRNA in the LNP.

67. The LNP of any one of embodiments 1 to 66, wherein the LNP comprises DPG-PEG2K at a concentration between about 1.0 and about 1.5 g per gram of mRNA in the LNP.

68. The LNP of any one of embodiments 1 to 67, wherein the LNP comprises DSPE-PEG3.4K-antibody conjugate at a concentration between about 0.05 to 0.1 g per gram of mRNA in the LNP.

69 The LNP of any one of embodiments 1 to 63, wherein
(i) the cationic lipid has a concentration about 49.2 mol % of the LNP;
(ii) the cholesterol has a concentration about 39.4 mol % of the LNP;
(iii) the DSPC has a concentration about 9.8 mol % of the LNP; and
(iv) the DPG-PEG2K has a concentration about 1.5 mol % of the LNP.

70. The LNP of any one of embodiments 1 to 63, wherein
(i) the cationic lipid has a concentration about 49.2 mol % of the LNP;
(ii) the cholesterol has a concentration about 29.3 mol % of the LNP;
(iii) the DSPC has a concentration about 20.0 mol % of the LNP; and
(iv) the DPG-PEG2K has a concentration about 1.5 mol % of the LNP.

71. The LNP of any one of embodiments 1 to 63, wherein
(i) the cationic lipid has a concentration about 14.2 g/g mRNA in the LNP;
(ii) the cholesterol has a concentration about 4.64 g/g mRNA in the LNP;
(iii) the DSPC has a concentration about 2.37 g/g mRNA in the LNP;
(iv) the DPG-PEG2K has a concentration about 1.15 g/g mRNA in the LNP; and
(v) the DSPE-PEG3.4K-anti-CD8 antibody conjugate has a concentration about 0.084 g/g to 0.15 g/g mRNA in the LNP.

71.1 The LNP of any one of embodiments 1 to 63, wherein
(i) the cationic lipid has a concentration about 14.2 g/g mRNA in the LNP;
(ii) the cholesterol has a concentration about 3.45 g/g mRNA in the LNP;
(iii) the DSPC has a concentration about 4.84 g/g mRNA in the LNP;
(iv) the DPG-PEG2K has a concentration about 1.15 g/g mRNA in the LNP; and
(v) the DSPE-PEG3.4K-anti-CD8 antibody conjugate has a concentration about 0.084 g/g to 0.15 g/g mRNA in the LNP.

71a. The LNP of any one of embodiments 1 to 71.1, wherein the LNP comprises:
(i) ionizable cationic Lipid 15 having the structure below,

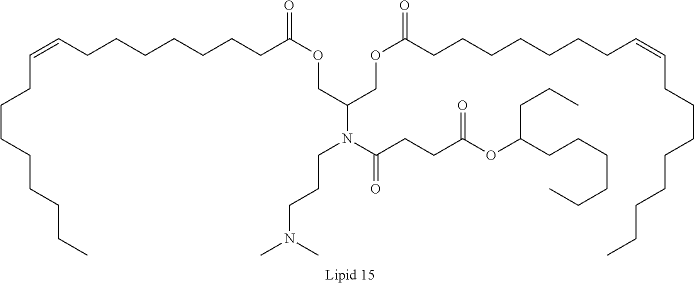 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof, or both;
(ii) cholesterol;
(iii) DSPC;
(iv) DPG-PEG;
(v) an mRNA comprising a nucleic acid sequence encoding a polypeptide comprising SEQ ID NO: 116, SEQ ID NO: 126, or SEQ ID NO: 127; and
(vi) a DSPE-PEG3.4K-anti-CD8α antibody conjugate, wherein the anti-CD8α antibody comprises SEQ ID NO: 9, SEQ ID NO: 169, or SEQ ID NO: 44.

71a.1. A lipid nanoparticle (LNP) comprising:
(a) a cationic lipid that is Lipid 15,

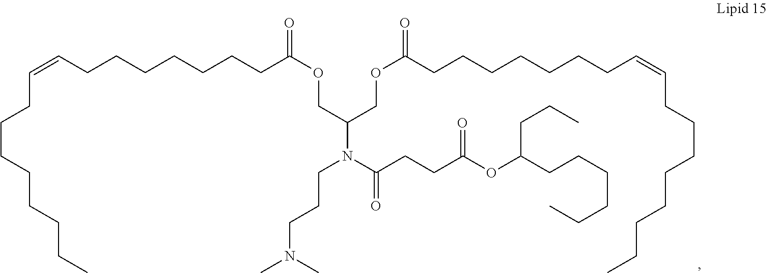 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof;
(b) cholesterol;
(c) 1,2-distearoyl-sn-glycero-3-phosphocholine (DSPC);
(d) PEG 2000-dipalmitoyl-glycerol (DPG-PEG2K);
(e) an mRNA comprising a nucleic acid sequence encoding a polypeptide comprising SEQ ID NO: 116, SEQ ID NO: 126, or SEQ ID NO: 127; and
(f) a DSPE-PEG3.4K-antibody conjugate, wherein the antibody comprises SEQ ID NO: 9, SEQ ID NO: 169, or SEQ ID NO: 44.

71b. The LNP of any one of embodiment 1 to embodiment 71a, wherein the LNP further comprises one or more additional components.

71c. The LNP of embodiment 71b, wherein the one or more additional components are included in the LNP due to a manufacturing process that is used to produce the LNP.

71d. The LNP of embodiment 71c, wherein at least one such additional component is a phospholipid-PEG that does not have a bioconjugation linker.

71e. The LNP of embodiment 71d, wherein the phospholipid-PEG is DSPE-PEG.

71f. The LNP of embodiment 71e, wherein the DSPE-PEG has a PEG with a molecular weight smaller than 3.4 kDa.

71 g. The LNP of embodiment 71f, wherein the DSPE-PEG has a PEG with a molecular weight of about 2.0 kDa.

71 h. The LNP of embodiment 71 g, wherein the DSPE-PEG2.0k has a concentration of less than 0.1 mol %, 0.09 mol %, 0.08 mol %, 0.07 mol %, 0.06 mol %, 0.05 mol %, 0.04 mol %, 0.03 mol %, 0.02 mol %, 0.01 mol %, 0.009 mol %, 0.008 mol %, 0.007 mol %, 0.006 mol %, 0.005 mol %, 0.004 mol %, 0.003 mol %, 0.002 mol %, or 0.001 mol % in the LNP, or a composition comprising the LNP, excluding solvent.

71i. The LNP of any one of embodiments 1 to 71 h, wherein the lipid-immune cell targeting group conjugate is presented in the LNP at a density from about 0.4 to 11.4, about 1.9 to 9.5, about 3.8 to 7.6, about 4.6 to 6.5, about 5.3 to 6.1, about 3.8, or about 5.7 micromoles of conjugate per gram of mRNA in the LNP, and optionally, the lipid-immune cell targeting group conjugate comprises SEQ ID NO: 9, SEQ ID NO: 169, or SEQ ID NO: 44.

71j. The LNP of any one of embodiments 1 to 71i, wherein the LNP further comprises one or more additional targeting moieties.

71k. The LNP of embodiment 71j, wherein at least one additional targeting moiety is an anti-CD4 antibody (e.g., Ibalizumab).

71l. The LNP of embodiment 71j or 71k, wherein the additional targeting moiety is in the same lipid-antibody conjugate for the anti-CD8 antibody, or in a different lipid-antibody conjugate.

71m. The LNP of embodiment 711, wherein the additional targeting moiety is in the same lipid-antibody conjugate for the anti-CD8 antibody, and the antibody in the conjugate is a bi-specific antibody targeting both CD8 and CD4.

71n. The LNP of embodiment 71m, wherein the additional targeting moiety is in a different lipid-antibody conjugate.

71o. The LNP of embodiment 71n, wherein the additional targeting moiety is an anti-CD4 antibody (e.g., Ibalizumab) in a different lipid-antibody conjugate.

71p. The LNP of embodiment 710, wherein the anti-CD8 conjugate and the anti-CD4 conjugate are presented in the LNP at a density of about 1.9 to 5.7 micromoles of conjugate per gram of mRNA in the LNP and about 1.9 to 11.4 micromoles of conjugate per gram of mRNA in the LNP respectively, and optionally the anti-CD8 conjugate has a density of about 1.9 micromoles of conjugate per gram of mRNA in the LNP and the anti-CD4 conjugate has a density of about 11.4 micromoles of conjugate per gram of mRNA in the LNP.

71q. The LNP of embodiment 1 to 71p, wherein a composition comprising the LNP has a dsRNA level (weigh %) of no greater than 0.1%, no greater than 0.09%, no greater than 0.08%, no greater than 0.07%, no greater than 0.06%, no greater than 0.05%, no greater than 0.04%, no greater than 0.03%, no greater than 0.02%, or no greater than 0.01% in the nucleic acids population.

72. An isolated polynucleotide that has the following formula, arranged from 5′ to 3′:
5′Cap (optional)-5′ UTR (optional)-nucleotides encoding a Lead peptide sequence (optional)-nucleotides encoding an antibody heavy chain variable region (VH)-nucleotides encoding a Linker A (optional)-nucleotides encoding an antibody light chain variable region (VL)-nucleotides encoding Linker B (optional)-nucleotides encoding a Hinge-nucleotides encoding a Transmembrane domain-nucleotides encoding Co-stimulatory domain-nucleotides encoding Signaling domain-3′ UTR (optional)-polyA tail (optional), wherein the VH and VL form a binding domain that specifically binds to human B-cell.

73. The isolated polynucleotide of embodiment 72, wherein the VH and VL forms a binding domain that specifically binds to human CD22.

74. The isolated polynucleotide of embodiment 73, wherein the VH, the Linker A, and VL form an anti-CD22 ScFv.

75. The isolated polynucleotide of embodiment 74, wherein the VH and VL domain comprises:
(1) a complementarity determining region-1 (CDR1), a CDR2, and a CDR3 of a heavy chain variable region (VH) comprising the amino acid sequence of SEQ ID NO: 433 and a CDR1, CDR2, and a CDR3 of a light chain variable region (VL) comprising the amino acid sequence of SEQ ID NO: 434;
(2) a complementarity determining region-1 (CDR1), a CDR2, and a CDR3 of a heavy chain variable region (VH) comprising the amino acid sequence of SEQ ID NO: 447 and a CDR1, CDR2, and a CDR3 of a light chain variable region (VL) comprising the amino acid sequence of SEQ ID NO: 448;
(3) a complementarity determining region-1 (CDR1), a CDR2, and a CDR3 of a heavy chain variable region (VH) comprising the amino acid sequence of SEQ ID NO: 457 and a CDR1, CDR2, and a CDR3 of a light chain variable region (VL) comprising the amino acid sequence of SEQ ID NO: 458;
(4) a complementarity determining region-1 (CDR1), a CDR2, and a CDR3 of a heavy chain variable region (VH) comprising the amino acid sequence of SEQ ID NO: 481 and a CDR1, CDR2, and a CDR3 of a light chain variable region (VL) comprising the amino acid sequence of SEQ ID NO: 482;
(5) a complementarity determining region-1 (CDR1), a CDR2, and a CDR3 of a heavy chain variable region (VH) comprising the amino acid sequence of SEQ ID NO: 495 and a CDR1, CDR2, and a CDR3 of a light chain variable region (VL) comprising the amino acid sequence of SEQ ID NO: 496;
(6) a complementarity determining region-1 (CDR1), a CDR2, and a CDR3 of a heavy chain variable region (VH) comprising the amino acid sequence of SEQ ID NO: 509 and a CDR1, CDR2, and a CDR3 of a light chain variable region (VL) comprising the amino acid sequence of SEQ ID NO: 510;
(7) a complementarity determining region-1 (CDR1), a CDR2, and a CDR3 of a heavy chain variable region (VH) comprising the amino acid sequence of SEQ ID NO: 523 and a CDR1, CDR2, and a CDR3 of a light chain variable region (VL) comprising the amino acid sequence of SEQ ID NO: 524;
(8) a complementarity determining region-1 (CDR1), a CDR2, and a CDR3 of a heavy chain variable region (VH) comprising the amino acid sequence of SEQ ID NO: 525 and a CDR1, CDR2, and a CDR3 of a light chain variable region (VL) comprising the amino acid sequence of SEQ ID NO: 526;
(9) a complementarity determining region-1 (CDR1), a CDR2, and a CDR3 of a heavy chain variable region (VH) comprising the amino acid sequence of SEQ ID NO: 527 and a CDR1, CDR2, and a CDR3 of a light chain variable region (VL) comprising the amino acid sequence of SEQ ID NO: 528;
(10) a complementarity determining region-1 (CDR1), a CDR2, and a CDR3 of a heavy chain variable region (VH) comprising the amino acid sequence of SEQ ID NO: 529 and a CDR1, CDR2, and a CDR3 of a light chain variable region (VL) comprising the amino acid sequence of SEQ ID NO: 530; or
(11) a complementarity determining region-1 (CDR1), a CDR2, and a CDR3 of a heavy chain variable region (VH) comprising the amino acid sequence of SEQ ID NO: 531 and a CDR1, CDR2, and a CDR3 of a light chain variable region (VL) comprising the amino acid sequence of SEQ ID NO: 532.

76. The isolated polynucleotide of embodiment 75, wherein the VH domain of the anti-CD22 ScFV comprises a CDR-H1 sequence comprising the amino acid sequence of SYGMH (SEQ ID NO: 427), a CDR-H2 sequence comprising the amino acid sequence of IIYYDGSKKYYADSVKG (SEQ ID NO: 428), and a CDR-H3 sequence comprising the amino acid sequence of ELTGDAFDI (SEQ ID NO: 429); and wherein the VL domain of the anti-CD22 ScFV comprises a CDR-L1 sequence comprising the amino acid sequence of RASQSIGSSLH (SEQ ID NO: 430), a CDR-L2 sequence comprising the amino acid sequence of YASQSFS (SEQ ID NO:431), and a CDR-L3 sequence comprising the amino acid sequence of HQSSTLPYT (SEQ ID NO: 432).

77. The isolated polynucleotide of embodiment 76, wherein the anti-CD22 ScFV comprises a VH domain comprising SEQ ID NO: 523, and a VL domain comprising SEQ ID NO: 524.

78. The isolated polynucleotide of any one of embodiment 72 to 77, wherein the VH domain and the VL domain is connected through a Linker A, wherein the Linker A is selected from the group consisting of SEQ ID Nos 337-348.

79. The isolated polynucleotide of any one of embodiment 72 to 78, wherein the Linker A is (GGGGS) 4 (SEQ ID NO: 344).

80. The isolated polynucleotide of any one of embodiment 72 to 79, wherein the Linker B is AS or AAA.

81. The isolated polynucleotide of any one of embodiment 72 to 80, wherein the hinge and transmembrane domains are derived from CD8 hinge and transmembrane domains.

82. The isolated polynucleotide of any one of embodiment 72 to 81, wherein the hinge and transmembrane domains have at least 80%, at least 85%, at least 90%, at least 95%, at least 96%, at least 97%, at least 98%, at least 98%, at least 99%, or 100% identity with SEQ ID NO: 538, SEQ ID NO: 539, or SEQ ID NO: 540.

83. The isolated polynucleotide of embodiment 82, wherein the hinge and transmembrane domains comprise SEQ ID NO: 538, SEQ ID NO: 539, or SEQ ID NO: 540.

84. The isolated polynucleotide of any one of embodiments 72 to 83, wherein the hinge and transmembrane domains are derived from CD28 hinge and transmembrane domains.

85. The isolated polynucleotide of embodiment 84, wherein the CD28 hinge and transmembrane domains have the amino acid sequence selected from the group consisting of (i) sequence of SEQ ID NO. 522; (ii) sequences that have at least 80% sequence identity with SEQ ID NO: 522; and (iii) sequences that have 3, 2, or 1 amino acid difference with SEQ ID NO: 522. 86. The isolated polynucleotide of any one of embodiments 72 to 85, wherein the Co-stimulatory domain is a CD28 Co-stimulatory domain.

87. The isolated polynucleotide of embodiment 86, wherein the CD28 Co-stimulatory domain has the amino acid sequence selected from the group consisting of (i) sequence of SEQ ID NO: 544; (ii) sequences that have at least 80% sequence identity with SEQ ID NO: 544; and (iii) sequences that have 3, 2, or 1 amino acid difference with SEQ ID NO: 544.

88. The isolated polynucleotide of any one of embodiments 72 to 87, wherein the Signaling domain is derived from a CD3z signaling domain.

89. The isolated polynucleotide of any one of embodiments 72 to 88, wherein the Signaling domain comprises or consists of SEQ ID NO: 544.

90. The isolated polynucleotide of any one of embodiments 72 to 89, wherein a polypeptide encoded by the polynucleotide comprises or consists of SEQ ID NO: 116, SEQ ID NO: 126, or SEQ ID NO: 127.

91. The isolated polynucleotide of any one of embodiments 72 to 90, wherein the polynucleotide encoding a polypeptide comprises a sequence encoding the polypeptide of SEQ ID NO: 116, SEQ ID NO: 126, or SEQ ID NO: 127.

92. The isolated polynucleotide of any one of embodiments 72 to 91, wherein the polynucleotide comprises SEQ ID NO: 108, SEQ ID NO: 124, or SEQ ID NO: 125, or the corresponding DNA sequence.

93. The isolated polynucleotide of any one of embodiments 72 to 92, wherein the nucleic acid sequence comprises SEQ ID NO: 139 or SEQ ID NO: 147, or the corresponding DNA sequence.

94. The isolated polynucleotide of any one of embodiments 72 to 93, wherein the nucleic acid comprises pseudouridine.

95. The isolated polynucleotide of embodiment 94, wherein the pseudouridine is N1-methyl-pseudouridine.

95a. The isolated polynucleotide of any one of embodiments 72 to 95, wherein the isolated polynucleotide is a DNA molecule.

95b. The isolated polynucleotide of any one of embodiments 72 to 95, wherein the isolated polynucleotide is an RNA molecule.

95c. The isolated polynucleotide of embodiment 95b, wherein the isolated polynucleotide is an mRNA molecule.

96. An expression construct comprising a polynucleotide of any one of embodiments 72 to 95c.

97. A vector, comprising the expression construction of embodiment 96.

98. A host cell comprising the expression construct of embodiment 96 or the vector of embodiment 97.

99. An in vitro transcribed mRNA derived from the isolated polynucleotide of any one of embodiments 72 to 95c.

100. An immune cell comprising the in vitro transcribed mRNA of embodiment 99.

101. A recombinant polypeptide encoded by the isolated polynucleotide of any one of embodiments 72 to 95c.

102. An immune cell expressing the recombinant polypeptide of embodiment 101.

103. A method of producing a polypeptide of interest in a cell, tissue or bodily fluid of a subject, the method comprising using the isolated polynucleotide of any one of embodiments 72 to 95c.

104. A method of preparing a LNP, comprising combining the isolated polynucleotide of any one of embodiments 72 to 95c with mixture of lipids.

105. A pharmaceutical composition, comprising a LNP of any one of embodiments 1 to 71q, the isolated polynucleotide of any one of embodiments 72 to 95c, the expression construct of embodiment 96, the vector of embodiment 97, the host cell of embodiment 98, and/or the recombinant polypeptide of embodiment 101.

106. A method of delivering a nucleic acid sequence into a human cell, comprising using the LNP of any one of embodiments 1 to 71q, wherein the LNP comprises the nucleic acid sequence to be delivered.

107. A method of modulating immune response in a human subject, comprising administering the LNP of any one of embodiments 1 to 71q, the isolated polynucleotide of any one of embodiments 72 to 95c, the expression construct of embodiment 96, the vector of embodiment 97, and/or the host cell of embodiment 98 to the human subject, and/or expressing the recombinant polypeptide of embodiment 101 in the human subject.

108. A method of treating B-cell malignancy in a human subject comprising administering the LNP of any one of embodiments 1 to 71q, the isolated polynucleotide of any one of embodiments 72 to 95c, the expression construct of embodiment 96, the vector of embodiment 97, and/or the host cell of embodiment 98 to the human subject, and/or expressing the recombinant polypeptide of embodiment 101 in the human subject.

109. The method of embodiment 108, wherein the B-cell malignancy is a B-cell lymphoma.

110. The method of embodiment 109, wherein the B-cell lymphoma is diffuse large B-cell lymphoma (DLBCL).

111. Use of the LNP of any one of embodiments 1 to 71q, the isolated polynucleotide of any one of embodiments 72 to 95c, the expression construct of embodiment 96, the vector of embodiment 97, the host cell of embodiment 98, and/or the pharmaceutical composition of embodiment 105 for the manufacture of a medicament for the treatment of a B-cell malignancy.

112. Use of the LNP of any one of embodiments 1 to 71q for the manufacture of a medicament for delivering a nucleic acid to a target cell.

113. The use of embodiment 112, wherein the target cell is an immune cell.

114.1 An immunoglobulin single variable domain (ISVD) specifically binding to human CD8alpha protein, that essentially consists of 4 framework regions (FR1 to FR4, respectively) and 3 complementarity determining regions (CDR1 to CDR3, respectively), wherein
CDR1 (according to Abm definition) comprises at least one amino acid residue selected from the group consisting of F27, T28, F29, E30, D31, Y32 and A33 (Kabat numbering), optionally at least two, at least three, at least four, at least five, or at least six amino acid residues selected from the group consisting of F27, T28, F29, E30, D31, Y32 and A33 (Kabat numbering);
CDR2 (according to Abm definition) comprises at least one amino acid residue selected from the group consisting of I51, R⁵², T52a, Y53, D54, E55, Q56, T57 and Y58, optionally at least two, at least three, at least four, at least five, at least six, at least seven, or at least eight amino acid residues selected from the group consisting of I51, R⁵², T52a, Y53, D54, E55, Q56, T57 and Y58 (Kabat numbering);
and/or
CDR3 (according to Abm definition) comprises at least one amino acid residue selected from the group consisting of: G95, S96, Y97, Y98, A99, C₁₀₀, A100a, Y100b, E100j, G100k, V100m, D100n, L100o, and D101 (Kabat numbering), optionally at least two, at least three, at least four, at least five, at least six, at least seven, at least eight, at least nine, at least ten, at least eleven, at least twelve, at least thirteen, at least fourteen, or at least fifteen amino acid residues selected from the group consisting of G95, S96, Y97, Y98, A99, C100, A100a, Y100b, E100j, G100k, V100m, D100n, L100o, and D101 (Kabat numbering);

114.2 The immunoglobulin single variable domain (ISVD) according to embodiment 114.1, wherein
CDR1 comprises at least the amino acid residues:
   D31;
   E30 and D31;
   E30, D31 and Y32;
   or
   E30, D31, Y32 and A33;
CDR2 comprises at least the amino acid residues:
   R⁵² and Y53;
   R⁵² and Q56;
   or
   R⁵², Y53, D54, Q56 and Y58;
and
CDR3 comprises at least the amino acid residues:
   D101;
   S96, Y97, D100n, and E100j;
   Y98;
   S96, Y97, E100j and D101;
   S96, Y97, D100n, E100j and D101;
   G95, S96, Y97, Y98, A99, C100, and A100a;
   G95, S96, Y97, Y98, A99, C100, A100a, and Y100b;
   or
   G95, S96, Y97, Y98, A99, C100, A100a, Y100b, E100j, G100k, V100m, D100n, L100o, and D101.

114.3 The immunoglobulin single variable domain (ISVD) of any of embodiments 114.1 or 114.2, wherein CDR2 comprises cysteine at position 50 (C50) and CDR3 comprises cysteine at position 100 (C100) and wherein amino acid residues C50 and C100 are covalently linked via a disulfide bond.

114.4 The immunoglobulin single variable domain (ISVD) according to any of embodiments 114.1 to 114.3, wherein CDR3 (according to Abm definition) comprises at least 20 amino acids, at least 21 amino acids, at least 22 amino acids, at least 23 amino acids, and/or wherein CDR3 (according to Abm definition) consists of 23 amino acids; and wherein position 100j (Kabat numbering) is E.

114.5 An immunoglobulin single variable domain (ISVD) specifically binding human CD8alpha, that essentially consists of 4 framework regions (FR1 to FR4, respectively) and 3 complementarity determining regions (CDR1 to CDR3, respectively), in which:
CDR1 (according to AbM definition) has an amino acid sequence selected from the group consisting of:
a) the amino acid sequence of SEQ ID NO: 244;
b) amino acid sequences that have at least 80% amino acid identity with the amino acid sequence of SEQ ID NO: 244; and
c) amino acid sequences that have 3, 2, or 1 amino acid difference with the amino acid sequences of SEQ ID NO: 244;
and
CDR2 (according to AbM definition) has an amino acid sequence selected from the group consisting of:
d) the amino acid sequence of SEQ ID NO: 246;
e) amino acid sequences that have at least 80% amino acid identity with the amino acid sequence of SEQ ID NO: 246; and
f) amino acid sequences that have 3, 2, or 1 amino acid difference with the amino acid sequence of SEQ ID NO: 246;
and
CDR3 (according to AbM definition) has an amino acid sequence selected from the group consisting of:
g) the amino acid sequence of SEQ ID NO: 248;
h) amino acid sequences that have at least 80% amino acid identity with the amino acid sequence of SEQ ID NO: 248; and
i) amino acid sequences that have 3, 2, or 1 amino acid difference with the amino acid sequence of SEQ ID NO: 248.

114.6 The immunoglobulin single variable domain (ISVD) specifically binding human CD8alpha according to embodiment 114.5, that essentially consists of 4 framework regions (FR1 to FR4, respectively) and 3 complementarity determining regions (CDR1 to CDR3, respectively), in which:
CDR1 (according to Abm definition) has an amino acid sequence selected from the group consisting of:
   a) the amino acid sequence of SEQ ID NO: 244;
   b) amino acid sequences that have at least 80% amino acid identity with the amino acid sequence of SEQ ID NO: 244; and
   c) amino acid sequences that have 3, 2, or 1 amino acid difference with the amino acid sequences of SEQ ID NO: 244;
   wherein CDR1 comprises at least three amino acid residues selected from the group consisting of F27, T28, F29, E30, D31, Y32 and A33 (Kabat numbering), optionally at least four, at least five, or at least six, or all amino acid residues selected from the group consisting of F27, T28, F29, E30, D31, Y32 and A33 (Kabat numbering);
and
CDR2 (according to Abm definition) has an amino acid sequence selected from the group consisting of:
   d) the amino acid sequence of SEQ ID NO: 246;
   e) amino acid sequences that have at least 80% amino acid identity with the amino acid sequence of SEQ ID NO: 246; and
   f) amino acid sequences that have 3, 2, or 1 amino acid difference with the amino acid sequence of SEQ ID NO: 246;
   wherein CDR2 comprises at least five amino acid residues selected from the group consisting of I51, R⁵², T52a, Y53, D54, E55, Q56, T57 and Y58 (Kabat numbering), optionally at least six, at least seven, at least eight, or all amino acid residues selected from the group consisting of I51, R⁵², T52a, Y53, D54, E55, Q56, T57 and Y58 (Kabat numbering);
and
CDR3 (according to Abm definition) has an amino acid sequence selected from the group consisting of:
   g) the amino acid sequence of SEQ ID NO: 248;
   h) amino acid sequences that have at least 80% amino acid identity with the amino acid sequence of SEQ ID NO: 248; and
   i) amino acid sequences that have 7, 6, 5, 4, 3, 2, or 1 amino acid difference with the amino acid sequence of SEQ ID NO: 248;
   wherein CDR3 comprises at least 7 amino acid residues selected from the group consisting of G95, S96, Y97, Y98, A99, C100, A100a, Y100b, E100j, G100k, V100m, D100n, L100o, and D101 (Kabat numbering), optionally at least eight, at least nine, at least ten, at least eleven, at least twelve, at least thirteen, or all amino acid residues selected from the group consisting of G95, S96, Y97, Y98, A99, C100, A100a, Y100b, E100j, G100k, V100m, D100n, L100o, and D101 (Kabat numbering).

114.7 The immunoglobulin single variable domain (ISVD) according to any of embodiment 114.5 or 114.6, wherein
CDR1 comprises at least the amino acid residues:
   D31;
   E30 and D31;
   E30, D31 and Y32;
   or
   E30, D31, Y32 and A33;
CDR2 comprises at least the amino acid residues:
   R⁵² and Y53;
   R⁵² and Q56;
   or
   R⁵², Y53, D54, Q56 and Y58;
and
CDR3 comprises at least the amino acid residues:
   D101;
   S96, Y97, D100n, and E100j;
   Y98;
   S96, Y97, E100j and D101;
   S96, Y97, D100n, E100j and D101;
   G95, S96, Y97, Y98, A99, C100, and A100a;
   G95, S96, Y97, Y98, A99, C100, A100a, and Y100b;
   or
   G95, S96, Y97, Y98, A99, C100, A100a, Y100b, E100j, G100k, V100m, D100n, L100o, and D101.

114.8 The immunoglobulin single variable domain (ISVD) according to any of embodiments 114.5 to 114.7, wherein CDR2 comprises cysteine at position 50 (C50) and CDR3 comprises cysteine at position 100 (C100) and wherein amino acid residues C50 and C100 are covalently linked via a disulfide bond.

114.9 The immunoglobulin single variable domain (ISVD) according to any of embodiments 114.5 to 114.8, wherein CDR3 (according to Abm definition) comprises at least 20 amino acids, at least 21 amino acids, at least 22 amino acids, at least 23 amino acids, and/or wherein CDR3 (according to Abm definition) consists of 23 amino acids; and wherein position 100j (Kabat numbering) is E.

114.10 The immunoglobulin single variable domain (ISVD) specifically binding human CD8alpha according to embodiment 114.5, that essentially consists of 4 framework regions (FR1 to FR4, respectively) and 3 complementarity determining regions (CDR1 to CDR3, respectively), in which:
CDR1 (according to Abm definition) has an amino acid sequence selected from the group consisting of:
   a) the amino acid sequence of GFTFX₁DYAIG (SEQ ID NO: 571);
   b) amino acid sequences that have at least 80% amino acid identity with the amino acid sequence of GFTFX₁DYAIG (SEQ ID NO: 571); and
c) amino acid sequences that have 3, 2, or 1 amino acid difference with the amino acid sequences of GFTFX₁DYAIG (SEQ ID NO: 571);
   wherein X₁ is selected from D and E;
and
CDR2 (according to Abm definition) has an amino acid sequence selected from the group consisting of:
   d) the amino acid sequence of CIRTYDX₂X₃TY (SEQ ID NO: 572);
   e) amino acid sequences that have at least 80% amino acid identity with the amino acid sequence of CIRTYDX₂X₃TY (SEQ ID NO: 572); and
   f) amino acid sequences that have 3, 2, or 1 amino acid difference with the amino acid sequence of CIRTYDX₂X₃TY (SEQ ID NO: 572)
   wherein X₂ is selected from G and E;
   wherein X₃ is selected from N and Q;
and
CDR3 (according to Abm definition) has an amino acid sequence selected from the group consisting of:
   g) the amino acid sequence of GSYYACAX₄YSRPDPSEX₅HVDX₆DY (SEQ ID NO: 573);
h) amino acid sequences that have at least 80% amino acid identity with the amino acid sequence of GSYYACAX₄YSRPDPSEX₅HVDX₆DY (SEQ ID NO: 573); and
i) amino acid sequences that have 3, 2, or 1 amino acid difference with the amino acid sequence of GSYYACAX₄YSRPDPSEX₅HVDX₆DY (SEQ ID NO: 573);
   wherein X₄ is selected from K, E and Y;
   wherein X₅ is selected from N and G; and/or wherein X₆ is selected from M and L.

115. The ISVD according to any of embodiments 114.5 to 114.10, in which the amino acid sequences of the CDRs (according to AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from the group consisting of SEQ ID NOs: 169 and SEQ ID NOs: 160 to 168, SEQ ID NOs: 28-36 and 44, and SEQ ID NOs: 10 to 27.

116. The ISVD according to any of embodiments 114.5 to 114.10 or 115, in which
CDR1 consists of the amino acid sequence of SEQ ID NO: 244;
CDR2 consists of the amino acid sequence of SEQ ID NO: 246; and
CDR3 consists of the amino acid sequence of SEQ ID NO: 248.

117.1. An immunoglobulin single variable domain (ISVD) specifically binding human CD8alpha, that essentially consists of 4 framework regions (FR1 to FR4, respectively) and 3 complementarity determining regions (CDR1 to CDR3, respectively), in which:
CDR1 (according to Kabat definition) has an amino acid sequence selected from the group consisting of:
a) the amino acid sequence of SEQ ID NO: 314;
b) amino acid sequences that have at least 80% amino acid identity with the amino acid sequence of SEQ ID NO: 314; and
c) amino acid sequences that have 3, 2, or 1 amino acid difference with the amino acid sequences of SEQ ID NO: 314;
and
CDR2 (according to Kabat definition) has an amino acid sequence selected from the group consisting of:
d) the amino acid sequence of SEQ ID NO: 316;
e) amino acid sequences that have at least 80% amino acid identity with the amino acid sequence of SEQ ID NO: 316; and
f) amino acid sequences that have 3, 2, or 1 amino acid difference with the amino acid sequence of SEQ ID NO: 316;
and
CDR3 (according to Kabat definition) has an amino acid sequence selected from the group consisting of:
g) the amino acid sequence of SEQ ID NO: 318;
h) amino acid sequences that have at least 80% amino acid identity with the amino acid sequence of SEQ ID NO: 318;
i) amino acid sequences that have 3, 2, or 1 amino acid difference with the amino acid sequence of SEQ ID NO: 318.

117.2 The immunoglobulin single variable domain (ISVD) specifically binding human CD8alpha according to embodiment 117.1, that essentially consists of 4 framework regions (FR1 to FR4, respectively) and 3 complementarity determining regions (CDR1 to CDR3, respectively), in which:

CDR1 (according to Kabat definition) has an amino acid sequence selected from the group consisting of:
   a) the amino acid sequence of SEQ ID NO: 314;
   b) amino acid sequences that have at least 80% amino acid identity with the amino acid sequence of SEQ ID NO: 314; and
   c) amino acid sequences that have 3, 2, or 1 amino acid difference with the amino acid sequences of SEQ ID NO: 314;
   wherein CDR1 comprises at least one amino acid residues selected from the group consisting of D31, Y32 and A33 (Kabat numbering), optionally at least two, or all three amino acid residues selected from the group consisting of D31, Y32 and A33 (Kabat numbering);
and
CDR2 (according to Kabat definition) has an amino acid sequence selected from the group consisting of:
   d) the amino acid sequence of SEQ ID NO: 316;
   e) amino acid sequences that have at least 80% amino acid identity with the amino acid sequence of SEQ ID NO: 316; and
   f) amino acid sequences that have 5, 4, 3, 2, or 1 amino acid difference with the amino acid sequence of SEQ ID NO: 316;
   wherein CDR2 comprises at least three amino acid residues selected from the group consisting of I51, R⁵², T52a, Y53, D54, E55, Q56, T57 and Y58 (Kabat numbering), optionally at least four, at least five, at least six, at least seven, at least eight, or all amino acid residues selected from the group consisting of I51, R⁵², T52a, Y53, D54, E55, Q56, T57 and Y58 (Kabat numbering);
and
CDR3 (according to Kabat definition) has an amino acid sequence selected from the group consisting of:
   g) the amino acid sequence of SEQ ID NO: 318;
   h) amino acid sequences that have at least 80% amino acid identity with the amino acid sequence of SEQ ID NO: 318;
   i) amino acid sequences that have 3, 2, or 1 amino acid difference with the amino acid sequence of SEQ ID NO: 318.
   wherein CDR3 comprises at least 7 amino acid residues selected from the group consisting of G95, S96, Y97, Y98, A99, C₁₀₀, A100a, Y100b, E100j, G100k, V100m, D100n, L1000, and D101 (Kabat numbering), optionally at least eight, at least nine, at least ten, at least eleven, at least twelve, at least thirteen, or all amino acid residues selected from the group consisting of G95, S96, Y97, Y98, A99, C100, A100a, Y100b, E100j, G100k, V100m, D100n, L100o, and D101 (Kabat numbering).

117.3. The immunoglobulin single variable domain (ISVD) according to embodiment 117.2, wherein
CDR1 comprises at least the amino acid residues:
   D31;
   D31 and Y32;
   or
   D31, Y32 and A33;
CDR2 comprises at least the amino acid residues:
   R⁵² and Y53;
   R⁵² and Q56;
   or
   R⁵², Y53, D54, Q56 and Y58;
and
CDR3 comprises at least the amino acid residues:
   D101;
   S96, Y97, D100n, and E100j;
   Y98;
   S96, Y97, E100j and D101;
   S96, Y97, D100n, E100j and D101;
   G95, S96, Y97, Y98, A99, C100, and A100a;
   G95, S96, Y97, Y98, A99, C100, A100a, and Y100b;
   or
   G95, S96, Y97, Y98, A99, C100, A100a, Y100b, E100j, G100k, V100m, D100n, L100o, and D101.

117.4 The immunoglobulin single variable domain (ISVD) according to any of embodiments 117.1 to 117.3, wherein CDR2 comprises cysteine at position 50 (C50) and CDR3 comprises cysteine at position 100 (C100) and wherein amino acid residues C50 and C100 are covalently linked via a disulfide bond.

117.5 The immunoglobulin single variable domain (ISVD) according to any of embodiments 117.1 to 117.4, wherein CDR3 (according to Kabat definition) comprises at least 20 amino acids, at least 21 amino acids, at least 22 amino acids, at least 23 amino acids, and/or wherein CDR3 (according to Kabat definition) consists of 23 amino acids; and wherein position 100j (Kabat numbering) is E.

117.6 The immunoglobulin single variable domain (ISVD) specifically binding human CD8alpha according to embodiment 117.1, that essentially consists of 4 framework regions (FR1 to FR4, respectively) and 3 complementarity determining regions (CDR1 to CDR3, respectively), in which:
CDR1 (according to Kabat definition) has an amino acid sequence selected from the group consisting of:
   a) the amino acid sequence of SEQ ID NO: 314;
   b) amino acid sequences that have at least 80% amino acid identity with the amino acid sequence of SEQ ID NO: 314; and
   c) amino acid sequences that have 3, 2, or 1 amino acid difference with the amino acid sequences of SEQ ID NO: 314;
and
CDR2 (according to Kabat definition) has an amino acid sequence selected from the group consisting of:
   d) the amino acid sequence of CIRTYDX₁X₂TYYX₃DSVKG (SEQ ID NO: 574);
   e) amino acid sequences that have at least 80% amino acid identity with the amino acid sequence of CIRTYDX₁X₂TYYX₃DSVKG (SEQ ID NO: 574); and
   f) amino acid sequences that have 3, 2, or 1 amino acid difference with the amino acid sequence of CIRTYDX₁X₂TYYX₃DSVKG (SEQ ID NO: 574);
   wherein X₁ is selected from G and E;
   wherein X₂ is selected from N and Q;
   wherein X₃ is selected from I and A;
and
CDR3 (according to Kabat definition) has an amino acid sequence selected from the group consisting of:
   g) the amino acid sequence of GSYYACAX₄YSRPDPSEX₅HVDX₆DY (SEQ ID NO: 573);
   h) amino acid sequences that have at least 80% amino acid identity with the amino acid sequence of GSYYACAX₄YSRPDPSEX₅HVDX₆DY (SEQ ID NO: 573); and
   i) amino acid sequences that have 3, 2, or 1 amino acid difference with the amino acid sequence of GSYYACAX₄YSRPDPSEX₅HVDX₆DY (SEQ ID NO: 573);
   wherein X₄ is selected from K, E and Y;
   wherein X₅ is selected from N and G; and/or wherein X₆ is selected from M and L.

118. The ISVD according to any of embodiments 117.1 to 117.6, in which the amino acid sequences of the CDRs (according to Kabat definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from the group consisting of SEQ ID NO: 179 and SEQ ID NOs 170 to 178.

119. The ISVD according to any one of embodiments 117.1 to 118, in which
CDR1 consists of the amino acid sequence of SEQ ID NO: 314;
CDR2 consists of the amino acid sequence of SEQ ID NO: 316; and
CDR3 consists of the amino acid sequence of SEQ ID NO: 318.

120. The ISVD according to any of one embodiments 114.1 to 119, wherein amino acid sequence has 80% amino acid sequence identity with one of the amino acid sequences of SEQ ID NO: 169, SEQ ID Nos: 160 to 168, and SEQ ID Nos: 28-36 and 44, or any one of SEQ ID Nos: 10 to 27, in which for the purposes of determining the degree of amino acid identity, the amino acid residues that form the CDR sequences are disregarded;
and wherein preferably one or more of the amino acid residues at positions 11, 37, 44, 45, 47, 83, 84, 103, 104 and 108 according to the Kabat numbering are chosen from the Hallmark residues mentioned in Table A.

121. The ISVD according to any one of embodiments 114.1 to 120, that essentially consists of a heavy chain variable domain sequence that is derived from a conventional four-chain antibody or that essentially consist of a heavy chain variable domain sequence that is derived from heavy chain antibody.

122. The ISVD according to any one of embodiments 114.1 to 121, that essentially consists of a VHH, a humanized VHH, a camelized VH, a domain antibody, a single domain antibody, or a dAb, or any combination thereof.

123. The ISVD according to any one of embodiments 114.1 to 122, which is a humanized ISVD that is chosen from the group consisting of SEQ ID NO: 9, SEQ ID NO: 169 (EVQLVESGGGVVQPGGSLRLSCAASGFTFEDYAIGWFRQAPGKEREEVSCIRTYDEQTY YADSVKGRFTISRDNAKNTVSLQMNSLRPEDTALYYCAAGSYYACAYYSRPDPSEGHV DLDYWGQGTLVTVSS) and SEQ ID NOs 160 to 168, and SEQ ID NOs 28-36 and 44, or any one of SEQ ID Nos: 10 to 27, or from the group consisting of amino acid sequences that have more than 80%, preferably more than 90%, more preferably more than 95%, such as 99% or more amino acid sequence identity with at least one of the amino acid sequences of SEQ ID NO: 9, SEQ ID NO: 169 and SEQ ID NO 160 to 168, and SEQ ID NOs 28-36 and 44, or any one of SEQ ID Nos: 10 to 27.

124. The ISVD according to any one of embodiments 114.1 to 123, in which the amino acid sequence is chosen from the group consisting of SEQ ID NO: 9, SEQ ID NO: 169 and SEQ ID NOs: 160 to 168, and SEQ ID NOs: 28-36 and 44, or any one of SEQ ID Nos: 10 to 27.

125.1 An immunoglobulin single variable domain (ISVD) specifically binding to human CD8alpha protein, that essentially consists of 4 framework regions (FR1 to FR4, respectively) and 3 complementarity determining regions (CDR1 to CDR3, respectively), that forms an interaction site of less than 4 Å with one or more amino acid residues in CD8alpha selected from: R²⁵, K42, Q44, V45, L46, L47, S48, N49, P50, T51, S52, G53, C₅₄, L71, Y72, L73, S74, Q75, N76, K77, R⁹³, L94, G95, D96, T97, D98; optionally wherein the ISVD is according to any of claims 114.1 to 124.

125.2 The immunoglobulin single variable domain (ISVD) specifically binding to human CD8alpha protein according to embodiment 125.1, that essentially consists of 4 framework regions (FR1 to FR4, respectively) and 3 complementarity determining regions (CDR1 to CDR3, respectively), that that forms an interaction site of less than 4 Å with at least two, at least three, at least four, at least five, at least six, at least seven, at least eight, at least nine, or at least ten amino acid residues in CD8alpha selected from: R²⁵, K42, Q44, V45, L46, L47, S48, N49, P50, T51, S52, G53, C54, L71, Y72, L73, S74, Q75, N76, K77, R⁹³, L94, G95, D96, T97, D98.

125.3 The immunoglobulin single variable domain (ISVD) according to any one of embodiments 125.1 to 125.2, that forms an interaction site of less than 4 Å with one or more amino acid residues in CD8alpha selected from:
R²⁵, K42, and R⁹³;
R²⁵, V45, L47, S48, Q75, N76, D96, T97;
R²⁵, K42, L47, S48, Q75, N76, R⁹³, D96;
R²⁵, K42, V45, L47, S48, Q75, N76, R⁹³, D96, T97;
L46, P50;
R²⁵, L46, P50, Q75, G95, and D96;
R²⁵, Q44, L46, L47, S48, P50, S52, Q75, N76, R⁹³, L94, G95, and D96;
R²⁵, K42, Q44, L46, L47, S48, P50, T51, S52, Q75, N76, R⁹³, L94, G95, D96, T97;
region Q44-L46: Q44, V45, and L46;
region L47-C54: L47, S48, N49, P50, T51, S52, G53, and C54;
region L74-K77: S74, Q75, N76, and K77;
region L71-K77: L71, Y72, L73, S74, Q75, N76, and K77; and
region G95-D98: R⁹³, L94, G95, D96, T97, and D98.

125.4 The immunoglobulin single variable domain (ISVD) according to any one of embodiments 125.1 to 125.3, that forms an interaction site of less than 4 Å with one or more amino acid residues in region L74-K77 of CD8alpha; optionally that forms an interaction site of less than 4 Å with N76.

125.5 The immunoglobulin single variable domain (ISVD) specifically binding human CD8alpha according to any one of embodiments 125.1 to 125.4, that comprises one or more of E30, D31, R⁵², Q56, S96, Y97, E100j, D100n, and D101, wherein upon binding CD8alpha, one or more of following interaction sites of less than 4 Å are formed selected from:
D31 (Kabat numbering) interacts with R²⁵ of CD8alpha;
D31 (Kabat numbering) interacts with K42 of CD8alpha;
D101 (Kabat numbering) interacts with R⁹³ of CD8alpha;
R⁵² (Kabat numbering) interacts with V45 of CD8alpha;
R⁵² (Kabat numbering) interacts with L47 of CD8alpha;
Q56 (Kabat numbering) interacts with S48 of CD8alpha;
S96 (Kabat numbering) interacts with Q75 of CD8alpha;
Y97 (Kabat numbering) interacts with D96 of CD8alpha;
E30 (Kabat numbering) interacts with R²⁵ of CD8alpha;
D100n (Kabat numbering) interacts with Q75 of CD8alpha;
E100j (Kabat numbering) interacts with N76 of CD8alpha.
D31 (Kabat numbering) interacts with R⁹⁶ of CD8alpha; and
D31 (Kabat numbering) interacts with T97 of CD8alpha.

125.6. The immunoglobulin single variable domain (ISVD) specifically binding human CD8alpha according to any one of embodiments 125.1 to 125.5, that comprises D31 and D101, wherein upon binding CD8alpha, following interaction sites of less than 4 Å are formed:
D31 (Kabat numbering) interacts with R²⁵ of CD8alpha;
D31 (Kabat numbering) interacts with K42 of CD8alpha;
D101 (Kabat numbering) interacts with R⁹³ of CD8alpha.

125.7. The immunoglobulin single variable domain (ISVD) specifically binding human CD8alpha according to any one of embodiments 125.1 to 125.6, that comprises E30, D31, R⁵², Q56, S96, Y97, E100j, and D100n, wherein upon binding CD8alpha, following interaction sites of less than 4 Å are formed:
R⁵² (Kabat numbering) interacts with V45 of CD8alpha;
R⁵² (Kabat numbering) interacts with L47 of CD8alpha;
Q56 (Kabat numbering) interacts with S48 of CD8alpha;
S96 (Kabat numbering) interacts with Q75 of CD8alpha;
Y97 (Kabat numbering) interacts with D96 of CD8alpha;
E30 (Kabat numbering) interacts with R²⁵ of CD8alpha;
D100n (Kabat numbering) interacts with Q75 of CD8alpha;
E100j (Kabat numbering) interacts with N76 of CD8alpha.
D31 (Kabat numbering) interacts with R⁹⁶ of CD8alpha; and
D31 (Kabat numbering) interacts with T97 of CD8alpha.

125.8 The immunoglobulin single variable domain (ISVD) specifically binding human CD8alpha according to embodiments 125.7, that comprises E100j, wherein upon binding CD8alpha, following interaction site of less than 4 Å is formed: E100j (Kabat numbering) interacts with N76 of CD8alpha.

126. The ISVD according to any one of embodiments 114 to 125.8, wherein the ISVD specifically binds to human CD8α with a dissociation constant (KD) of 5.10⁻⁹ to 10⁻¹¹ moles/litre or less, and preferably 10⁻⁹ to 5.10⁻¹¹ moles/litre or less and more preferably 5.10⁻¹⁰ to 10⁻¹⁰ moles/litre, as determined by Surface Plasmon Resonance.

127. The ISVD according to any one of embodiments 114 to 126, wherein the ISVD specifically binds to human CD8α with a kon-rate of between 10⁵ M⁻¹s⁻¹ to about 10⁷ M⁻¹s⁻¹, preferably between 5.10⁵ M⁻¹s⁻¹ and 10⁷ M⁻¹s⁻¹, more preferably between 10⁶ M⁻¹s⁻¹ and 10⁷ M⁻¹s⁻¹, such as between 10⁶ M⁻¹s⁻¹ and 5.10⁶ M⁻¹s⁻¹, as determined by Surface Plasmon Resonance.

128. The ISVD according to any one of embodiments 114 to 127, wherein the ISVD specifically binds to humanCD8α with a koff rate between 10⁻³ s⁻¹ (t½=0.69 s) and 10⁻⁶ s⁻¹ (providing a near irreversible complex with a t½ of multiple days), preferably between 10⁻³ s⁻¹ and 5.10⁻⁶ s⁻¹, more preferably between 5.10⁻⁴ s⁻¹ and 5.10⁻⁶ s⁻¹, such as between 5.10⁻⁴ s⁻¹ and 10⁻⁵ s⁻¹, as determined by Surface Plasmon Resonance.

129. The ISVD according to any one of embodiments 114 to 128, wherein the ISVD specifically binds to human and cyno CD8α and does not bind to other T-cell surface glycoproteins.

130. The ISVD according to any one of embodiments 114 to 129, wherein the ISVD antagonizes an activity of CD8α, CD8α homodimer, and/or CD8α/CD8β heterodimer.

131. The ISVD according to any one of embodiments 114 to 130, wherein the ISVD blocks the interaction of human CD8 co-receptor with human Major Histocompatibility Complex (MHC) class I protein with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁹ M, between 10⁻¹⁰ M and 10⁻⁸ M or between 10⁻¹¹ M and 10⁻⁹ M, for example, as measured in a FACS binding assay.

132. The ISVD according to any one of embodiments 114 to 131, wherein the ISVD blocks the interaction of cyno CD8 co-receptor with cyno Major Histocompatibility Complex (MHC) class I protein with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁹ M, between 10⁻¹⁰ M and 10⁻⁸ M or between 10⁻¹¹ M and 10⁻⁹ M, for example, as measured in a FACS binding assay.

133. The ISVD according to any one of embodiments 114 to 132, wherein the ISVD blocks the binding of hCD8 co-receptor to hMHC class I protein by at least 50%, such as at least 60%, 70%, 80%, 90%, 95%, 99% or even more, as determined by ligand competition, AlphaScreen, or competitive binding assays (such as competition ELISA or competition FACS).

134. The ISVD according to any one of embodiments 114 to 133, wherein the ISVD blocks the interaction of CD8 co-receptor with lymphocyte-specific protein tyrosine kinase with a potency (EC50 value) of 10⁻⁸ M or lower, more preferably of 10⁻⁹ M or lower, or even of 5.10⁻¹⁰ M or lower, such as between 10⁻¹¹ M and 10⁻⁸ M, between 10⁻¹⁰ M and 10⁻⁸ M, between 10⁻¹⁰ M and 10⁻⁹ M or between 10⁻¹¹ M and 10⁻⁹ M, as determined in a functional assay.

135. A polypeptide or construct that comprises or essentially consists of one or more ISVDs according to any one of embodiments 114 to 134, and optionally further comprises one or more other groups, residues, moieties or binding units, optionally linked via one or more linkers.

136. The polypeptide or construct according to embodiment 135, in which said one or more other groups, residues, moieties or binding units are amino acid sequences.

137. The polypeptide or construct according to any one of embodiments 135 to 136, in which said one or more linkers are one or more amino acid sequences.

138. The polypeptide or construct according to any one of embodiments 135 to 137, in which said one or more other groups, residues, moieties or binding units are immunoglobulin sequences.

139. The polypeptide or construct according to any one of embodiments 135 to 138, in which said one or more other groups, residues, moieties or binding units are ISVDs.

140. The polypeptide or construct according to any one of embodiments 135 to 139, in which said one or more other groups, residues, moieties or binding units are chosen from the group consisting of VHHs, humanized VHHs, camelized VHs, domain antibodies, single domain antibodies and dAbs.

141. The polypeptide or construct according to any one of embodiments 135 to 140, which is a multivalent construct.

142. The polypeptide or construct according to any one of embodiments 135 to 141, which is a multispecific construct.

143. The polypeptide or construct according to any one of embodiments 135 to 142, in which said one or more other groups, residues, moieties or binding units provide the polypeptide or construct with increased half-life, compared to the ISVD without the one or more other groups, residues, moieties or binding units.

144. The polypeptide or construct according to embodiment 143, in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.

145. The polypeptide or construct according to embodiment 144, in which said one or more other groups, residues, moieties or binding units that provide the polypeptide or construct with increased half-life is chosen from the group consisting of human serum albumin or fragments thereof.

146. The polypeptide or construct according to embodiment 145, in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).

147. The polypeptide or construct according to embodiment 146, in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).

148. The polypeptide or construct according to embodiment 147, in which said one or more other groups, residues, moieties or binding units that provides the polypeptide or construct with increased half-life is an ISVD that specifically binds human serum albumin.

149. The polypeptide or construct according to embodiment 148, wherein said ISVD that specifically binds human serum albumin essentially consists of 4 framework regions (FR1 to FR4, respectively) and 3 complementarity determining regions (CDR1 to CDR3 respectively), in which:
CDR1 (according to AbM definition) has an amino acid sequence selected from the group consisting of:
   a) the amino acid sequence of GFTFRSFGMS (SEQ ID NO:566);
   b) amino acid sequences that have at least 80% amino acid identity with the amino acid sequence of SEQ ID NO:566; and
   c) amino acid sequences that have 3, 2, or 1 amino acid difference with the amino acid sequences of SEQ ID NO:566;
and
CDR2 (according to AbM definition) has an amino acid sequence selected from the group consisting of:
   d) the amino acid sequence of SISGSGSDTL (SEQ ID NO: 567);
   e) amino acid sequences that have at least 80% amino acid identity with the amino acid sequence of SEQ ID NO:567;
   f) amino acid sequences that have 3, 2, or 1 amino acid difference with the amino acid sequence of SEQ ID NO:567;
and
CDR3 (according to AbM definition) has an amino acid sequence selected from the group consisting of:
   g) the amino acid sequence of GGSLSR (SEQ ID NOs: 568);
   h) amino acid sequences that have at least 80% amino acid identity with the amino acid sequence of SEQ ID NO:568;
   i) amino acid sequences that have 3, 2, or 1 amino acid difference with the amino acid sequence of SEQ ID NO:568.

150. The polypeptide or construct according to embodiment 149, wherein CDR1 consists of the amino acid sequence of SEQ ID NO:566, CDR2 consists of the amino acid sequence of SEQ ID NO: 567, and CDR3 consists of the amino acid sequence of SEQ ID NO:568.

151. The polypeptide or construct according to embodiment 150, wherein said ISVD that specifically binds human serum albumin is selected from the group consisting of ALB8 (SEQ ID NO: 320), ALB23 (SEQ ID NO: 321), ALBX00001 (SEQ ID NO: 334) and ALB23002 (SEQ ID NO: 335).

152. The polypeptide or construct according to anyone of embodiments 135 to 151, wherein said linker is chosen from the group consisting of SEQ ID NOs: 336 to 352.

153. The polypeptide or construct according to any of embodiments 135 to 152, further comprising a C-terminal extension.

154. The polypeptide or construct according to embodiment 153, wherein said C-terminal extension is a C-terminal extension (X)n, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

155. A nucleic acid that encodes an ISVD according to any one of embodiments 114 to 134, or a polypeptide according to any one of embodiments 134 to 153.

156. The nucleic acid according to embodiment 155, that is in the form of a genetic construct.

157. A non-human host or host cell that expresses, or that under suitable circumstances is capable of expressing, an ISVD according to any one of embodiments 114 to 134, or a polypeptide according to any one of embodiments 134 to 153; and/or that comprises the nucleic acid according to any one of embodiments 154 or 155.

158. A method for producing an ISVD according to any one of embodiments 114 to 134, or a polypeptide according to any one of embodiments 135 to 154, the method comprising:
a) expressing, in a suitable non-human host cell or host organism or in another suitable expression system, a nucleic acid according to any one of embodiments 155 or 156;
optionally followed by:
b) isolating and/or purifying the ISVD according to any one of embodiments 114 to 134, or the polypeptide according to any one of embodiments 135 to 154.

159. A method for producing an ISVD according to any one of embodiments 114 to 134, or a polypeptide according to any one of embodiments 135 to 154, said method comprising:
a) cultivating and/or maintaining a non-human host or host cell according to embodiment 157 under conditions that are such that said non-human host or host cell expresses and/or produces at least one ISVD according to any one of embodiments 114 to 134, or at least one polypeptide according to any one of embodiments 135 to 154;
optionally followed by:
b) isolating and/or purifying the ISVD according to any one of embodiments 114 to 134, or polypeptide according to any one of embodiments 135 to 154.

160. A composition comprising at least one ISVD according to any one of embodiments 114 to 133, at least one polypeptide or construct according to any one of embodiments 135 to 154, or at least one nucleic acid according to any one of embodiments 155 to 156, or any combination thereof.

161. The composition according to embodiment 160, which is a pharmaceutical composition.

162. The composition according to any one of embodiments 160 or 161, which is a pharmaceutical composition, that further comprises at least one pharmaceutically acceptable carrier, diluent or excipient and/or adjuvant, and that optionally comprises one or more further pharmaceutically active polypeptides and/or compounds.

163. The ISVD according to any one of embodiments 114 to 134, the polypeptide or construct according to any one of embodiments 134 to 154, or the composition according to any one of embodiments 160 to 162, for use as a medicament.

164. The ISVD according to any one of embodiments 114 to 134, the polypeptide or construct according to any one of embodiments 135 to 154, or the composition according to any one of embodiments 160 to 162, for use in the diagnosis, prevention and/or treatment of at least one disease and/or disorder.

165. The ISVD according to any one of embodiments 114 to 134, the polypeptide or construct according to any one of embodiments 135 to 154, or the composition according to any one of embodiments 160 to 162, for use in the diagnosis, prevention and/or treatment of at least one disease and/or disorder that is associated with CD8alpha, with its biological or pharmacological activity, and/or with the biological pathways or signaling in which CD8alpha is involved.

166. The ISVD according to any one of embodiments 114 to 134, the polypeptide or construct according to any one of embodiments 135 to 154, or the composition according to any one of embodiments 160 to 162, for use in the diagnosis, prevention and/or treatment of an immunological disease, an infectious disease, or a proliferative disease, such as B cell leukemias and lymphomas.

167. A method for the diagnosis, prevention and/or treatment of at least one disease and/or disorder, comprising the administration, to a subject, of an ISVD according to any one of embodiments 114 to 134, a polypeptide or construct according to any one of embodiments 135 to 154, or a composition according to any one of embodiments 160 to 162.

168. The method according to embodiment 167, for the diagnosis, prevention and/or treatment of at least one disease or disorder that is associated with CD8alpha, with its biological or pharmacological activity, and/or with the biological pathways or signaling in which CD8alpha is involved, said method comprising administering, to a subject, at least one ISVD according to any one of embodiments 113 to 134, a polypeptide or construct according to any one of embodiments 135 to 154, or composition according to any one of embodiments 160 to 162.

169. The method according to any one of embodiments 167 or 168, for the diagnosis, prevention and/or treatment of cancer, said method comprising administering, to a subject, at least one ISVD according to any one of embodiments 114 to 134, a polypeptide or construct according to any one of embodiments 135 to 155, or a composition according to any one of embodiments 160 to 162.

170. An immunoglobulin single variable domain (ISVD) specifically binding human CD8alpha, that essentially consists of 4 framework regions (FR1 to FR4, respectively) and 3 complementarity determining regions (CDR1 to CDR3, respectively), in which:
CDR1 (according to AbM definition) has an amino acid sequence selected from the group consisting of:
   a) the amino acid sequence of SEQ ID NO. 181 or 188;
   b) amino acid sequences that have at least 80% amino acid identity with the amino acid sequence of SEQ ID NO: 181 or 188; and
   c) amino acid sequences that have 3, 2, or 1 amino acid difference with the amino acid sequences of SEQ ID NO: 181 or 188;
      and
CDR2 (according to AbM definition) has an amino acid sequence selected from the group consisting of:
   d) the amino acid sequence of SEQ ID NO: 183 or 190;
   e) amino acid sequences that have at least 80% amino acid identity with the amino acid sequence of SEQ ID NO:183 or 190;
   f) amino acid sequences that have 3, 2, or 1 amino acid difference with the amino acid sequence of SEQ ID NO: 183 or 190;
      and
CDR3 (according to AbM definition) has an amino acid sequence selected from the group consisting of:
   g) the amino acid sequence of SEQ ID NO: 185, 192, 199 or 206;
   h) amino acid sequences that have at least 80% amino acid identity with the amino acid sequence of SEQ ID NO: 185, 192, 199 or 206;
   i) amino acid sequences that have 3, 2, or 1 amino acid difference with the amino acid sequence of SEQ ID NO: 185, 192, 199 or 206.

171. The ISVD of embodiment 170, wherein the ISVD is selected from the group consisting of:
a) an ISVD comprising SEQ ID NOs: 181, 183, and 185;
b) an ISVD comprising SEQ ID NOs: 188, 190, and 192;
c) an ISVD comprising SEQ ID NOs: 188, 190, and 199;
d) an ISVD comprising SEQ ID NOs: 188, 190, and 206;

172. The ISVD of any of embodiments 170 to 171, wherein the FR1 to FR4 in the ISVD is selected from the group consisting of:
a) an FR1 comprising SEQ ID NO: 180 or 187;
b) an FR2 comprising SEQ ID NO: 182;
c) an FR3 comprising SEQ ID NO: 184, 191, 198 or 233; and
d) an FR4 comprising SEQ ID NO: 186 or 193.

173. The ISVD of embodiment 172, wherein the ISVD is selected from the group consisting of:
a) an ISVD comprising SEQ ID NO: 160;
b) an ISVD comprising SEQ ID NO: 161;
c) an ISVD comprising SEQ ID NO: 162;
d) an ISVD comprising SEQ ID NO: 163;
e) an ISVD comprising SEQ ID NO: 164;
f) an ISVD comprising SEQ ID NO: 165;
g) an ISVD comprising SEQ ID NO: 166;
h) an ISVD comprising SEQ ID NO: 167; and
i) an ISVD comprising SEQ ID NO: 168.

174. An immunoglobulin single variable domain (ISVD) specifically binding human CD8alpha, that essentially consists of 4 framework regions (FR1 to FR4, respectively) and 3 complementarity determining regions (CDR1 to CDR3, respectively), in which:
CDR1 (according to Kabat definition) has an amino acid sequence selected from:
a) the amino acid sequence of SEQ ID NO: 251;
b) amino acid sequences that have at least 80% amino acid identity with the amino acid sequence of SEQ ID NO: 251;
c) amino acid sequences that have 3, 2, or 1 amino acid difference with the amino acid sequences of SEQ ID NO: 251;
and
CDR2 (according to Kabat definition) has an amino acid sequence selected from:
d) the amino acid sequence of SEQ ID NO: 253 or 260;
e) amino acid sequences that have at least 80% amino acid identity with the amino acid sequence of SEQ ID NO: 253 or 260;
f) amino acid sequences that have 3, 2, or 1 amino acid difference with the amino acid sequence of SEQ ID NO: 253 or 260;
and
CDR3 (according to Kabat definition) has an amino acid sequence selected from:
g) the amino acid sequence of SEQ ID NO: 255, 262, 269, or 276;
h) amino acid sequences that have at least 80% amino acid identity with the amino acid sequence of SEQ ID NO: 255, 262, 269, or 276;
i) amino acid sequences that have 3, 2, or 1 amino acid difference with the amino acid sequence of SEQ ID NO: 255, 262, 269, or 276.

175. The ISVD of embodiment 174 wherein the ISVD is selected from the group consisting of:
a) an ISVD comprising SEQ ID NOs: 251, 253, and 255;
b) an ISVD comprising SEQ ID NOs: 258, 260, and 262;
c) an ISVD comprising SEQ ID NOs: 265, 267, and 269; and
d) an ISVD comprising SEQ ID NOs: 272, 274, and 276;

176. The ISVD of embodiment 175, wherein the FR1 to FR4 in the ISVD is selected from the group consisting of:
a) an FR1 comprising SEQ ID NO: 250 or 257;
b) an FR2 comprising SEQ ID NO: 252;
c) an FR3 comprising SEQ ID NO: 254, 261, 282, or 303; and
d) an FR4 comprising SEQ ID NO: 256 or 263.

177. The ISVD of embodiment 176, wherein the ISVD is selected from the group consisting of:
a) an ISVD comprising SEQ ID NO: 170;
b) an ISVD comprising SEQ ID NO: 171;
c) an ISVD comprising SEQ ID NO: 172;
d) an ISVD comprising SEQ ID NO: 173;
e) an ISVD comprising SEQ ID NO: 174;
f) an ISVD comprising SEQ ID NO: 175;
g) an ISVD comprising SEQ ID NO: 176;
h) an ISVD comprising SEQ ID NO: 177; and
i) an ISVD comprising SEQ ID NO: 178;

178. The ISVD of any one of embodiments 114 to 134 or 168 to 177, wherein the ISVD has a cysteine containing linker at its C-terminal end.

179. The ISVD of embodiment 178, wherein the cysteine containing linker is a GGC linker.

180. The ISVD of any one of embodiments 114 to 179, wherein ISVD comprises a C-terminal extension sequence of any one of SEQ ID Nos: 353 to 371.

181. The ISVD of embodiment 180, wherein the C-terminal extension consists of VTVSS(X)n (SEQ ID NO: 353).

182. The ISVD of embodiment 181, wherein the C-terminal extension consists of VTVSS (SEQ ID NO: 371).

183. A conjugate comprising an ISVD according to any one of embodiments 114 to 134 and 170 to 182 linked to a phospholipid-PEG-maleimide derivative.

184. The conjugate of embodiment 183, wherein the phospholipid-PEG-maleimide derivative is a derivative of phosphatidylethanolamine.

185. The conjugate of embodiment 183, wherein the phospholipid-PEG-maleimide derivative comprises stearic acid acyl chains.

186. The conjugate of any one of embodiments 183 to 185, wherein the phospholipid is 1,2-Distearoyl-sn-glycero-3-phosphoethanolamine-maleimide (DSPE).

187. The conjugate of any one of embodiments 183 to 186, wherein the PEG has a molecular weight from about 1.5 kDa to about 6 kDa.

188. The conjugate of any one of embodiments 183 to 187, wherein the PEG has a molecular weight of 2 kDa, 3.4 kDa or 5 kDa.

189. The conjugate of embodiment 188, wherein the PEG has a molecular weight of 3.4 kDa.

190. The conjugate of any one of embodiments 183 to 189, wherein the phospholipid-PEG-maleimide derivative is DSPE-PEG 3.4 K-maleimide.

191. A method for the preparation of a composition comprising monomers of an ISVD with a cysteine containing linker at its C-terminal end, the method comprising the following sequential steps:
(a) reducing a composition comprising ISVD dimers to ISVD monomers with a first reducing agent, wherein the ISVD dimers are formed through the cysteine containing linker at the C-terminal end of the ISVD;
(b) purifying the ISVD monomers obtained in step (a) to get a purified composition comprising the ISVD monomers;
(c) reducing the purified composition obtained in step (b) with a second reducing agent; and
(d) purifying the reduced composition obtained in step (c) to obtain a composition comprising monomers of the ISVD.

192. The method of embodiment 191, wherein the composition comprising the ISVD dimers in step (a) is obtained through expressing the ISVD in a host cell.

193. The method of embodiment 192, wherein the composition comprising the ISVD dimers is purified to remove host cell proteins and DNA before being subjected to step (a).

194. The method of any one of embodiments 191 to 193, wherein the first reducing agent comprises tris(2-carboxyethyl)phosphine (TCEP).

195. The method of any one of embodiments 191 to 194, wherein the step (a) is conducted around 15 to 25° C., optionally around 20-22° C.

196. The method of any one of embodiments 191 to 195, wherein the step (a) takes about 16 to 20 hours.

197. The method of any one of embodiments 191 to 196, wherein the step (b) comprises using a chromatography.

198. The method of embodiment 197, wherein the chromatography comprises an ion exchange chromatography (IEX).

199. The method of any one of embodiments 191 to 198, wherein the second reducing agent in step (c) comprises tris(2-carboxyethyl)phosphine (TCEP).

200. The method of any one of embodiments 191 to 199, wherein the step (c) is conducted around 15 to 25° C., optionally around 20-22° C. 201. The method of any one of embodiments 191 to 200, wherein the step (c) takes about 16 to 20 hours.

202. The method of any one of embodiments 191 to 201, wherein the step (d) comprises Ultrafiltration/Diafiltration (UF/DF).

203. The method of any one of embodiments 191 to 202, wherein at least 80% of the ISVD in the composition obtained in step (d) is in monomeric form.

204. The method of any one of embodiments 191 to 203, wherein the cysteine containing linker is a GGC linker.

205. The method of any one of embodiments 191 to 204, wherein the C-terminal end comprises the sequence VTVSS (SEQ ID NO: 371) before the cysteine linker.

206. The method of any one of embodiments 191 to 205, wherein the ISVD comprises two internal disulphide bridges.

206a. The method of any of embodiments 191 to 206, wherein the ISVD comprises a disulfide bridge between the cysteine residue at position 22 and the cysteine residue at position 92, and further comprises a disulfide bridge that is formed between a cysteine residue at position 50 and a cysteine residue in another CDR, such as CDR1, CDR2 or CDR3.

206b. The method of any of embodiments 191 to 205, wherein the ISVD comprises a disulfide bridge between the cysteine residue at position 22 and the cysteine residue at position 92, and further comprises a disulfide bridge that is formed between a cysteine residue at position 50 and a cysteine residue in CDR3.

206c. The method of any one of embodiments 191 to 205, wherein the ISVD is a VHH1 ISVD.

207. The method of any one of embodiments 193 to 206c, wherein, before being subjected to step (a), the ISVD dimers is purified using protein A chromatography to remove host cell proteins and DNA.

208. The method of any one of embodiments 193 to 207, wherein both the first reducing agent and second reducing agent comprise TCEP.

209. The method of any one of embodiments 193 to 208, wherein the first reducing agent comprises 20×TCEP.

210. The method of any one of embodiments 193 to 209, wherein the second reducing agent comprise 10×TCEP.

211. The method of any one of embodiments 202 to 209, wherein the UF/DF membrane has a molecular weight cut-off of 10 kDa.

212. A method for the preparation of a phospholipid-PEG-ISVD conjugate comprising the following sequential steps:
(a) mixing a first composition comprising monomers of an ISVD comprising a cysteine containing linker, with a second composition comprising phospholipid-PEG molecules comprising a bioconjugation linker under conditions that the phospholipid-PEG molecules and the ISVD monomers can form a conjugate through clicking chemistry; and
(b) adding cysteine to the conjugate obtained in step (a) under conditions that the conjugation reaction is quenched,
wherein a composition comprising the phospholipid-PEG ISVD conjugate is obtained.

213. The method of embodiment 212, wherein at least 80% of the ISVD in the first composition is in monomeric form.

214. The method of embodiment 212 or embodiment 213, wherein the phospholipid in the phospholipid-PEG is a derivative of phosphatidylethanolamine.

215. The method of any one of embodiments 212 to 214, wherein the phospholipid comprises stearic acid acyl chains.

216. The method of any one of claims 212 to 215, wherein the phospholipid is 1,2-Distearoyl-sn-glycero-3-phosphoethanolamine (DSPE).

217. The method of any one of embodiments 212 to 216, wherein the PEG has a molecular weight of about 1.5 kDa to about 6.5 kDa.

218. The method of embodiment 217, wherein the PEG has a molecular weight of about 2 kDa, about 3.4 kDa, or about 5 kDa.

219. The method of embodiment 218, wherein the PEG has a molecular weight of 3.4 kDa.

220. The method of any one of embodiments 212 to 219, wherein the conjugate is a DSPE-PEG 3.4K-ISVD conjugate.

221. The method of any one of embodiments 212 to 220, wherein the bioconjugation linker in the phospholipid-PEG has a maleimide group.

222. The method of any one of embodiments 212 to 221, wherein the second composition further comprises molecules of a second phospholipid-PEG that does not have the bioconjugation linker in addition to the phospholipid-PEG molecules comprising the bioconjugation linker.

223. The method of embodiment 212, wherein the size of PEG in the second phospholipid-PEG is different compared to the size of PEG in the phospholipid-PEG comprising the bioconjugation linker.

224. The method of embodiment 223, wherein the size of PEG in the second phospholipid-PEG is smaller compared to the size of PEG in the phospholipid-PEG comprising the bioconjugation linker.

225. The method of embodiment 224, wherein the size of PEG in the second phospholipid-PEG is about 2 kDa, and the size of PEG in the phospholipid-PEG comprising the bioconjugation linker is 3.4 KDa.

226. The method of embodiment 225, wherein the second composition comprises DSPE-PEG 3.4 kDa with a bioconjugation linker, and DSPE-PEG 2.0 kDa without the bioconjugation linker.

227. The method of embodiment 226, wherein the DSPE-PEG 2.0 kDa has the structure below or a salt thereof:

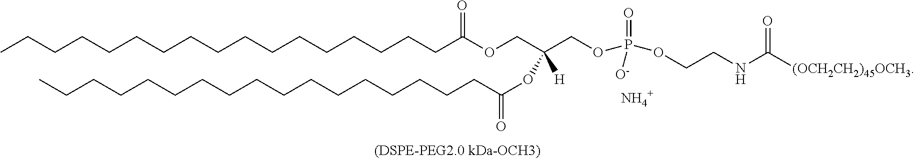

228. The method of embodiment 227, wherein the DSPE-PEG 3.4 kDa has a maleimide linker.

229. The method of any one of embodiments 212 to 228, wherein the cysteine containing linker in the ISVD is a GGC linker.

230. The method of embodiment 229, wherein the cysteine containing linker comprising a sequence of any one of SEQ ID Nos: 353 to 370.

231. The method of embodiment 230, wherein the ISVD comprising VTVSS(X)n (SEQ ID NO: 353) before the GGC linker.

232. The method of embodiment 231, wherein the ISVD comprising VTVSS (SEQ ID NO: 371) before the GGC linker.

233. The method of any one of embodiments 222 to 232, wherein the molar ratio of the phospholipid-PEG molecules comprising the bioconjugation linker to the second phospholipid-PEG that does not have the bioconjugation linker is about 1:3 to about 1:1.

234. The method of embodiment 233, wherein the molar ratio of the phospholipid-PEG molecules comprising the bioconjugation linker to the second phospholipid-PEG that does not have the bioconjugation linker is about 2:3.

235. The method of any one of embodiments 222 to 234, wherein the molar ratio of the ISVD monomers, the phospholipid-PEG molecules comprising the bioconjugation linker, and the second phospholipid-PEG that does not have the bioconjugation linker is about 1:1:4 or 1:2:3.

236. The method of any one of embodiments 212 to 235, wherein a clicking chemistry reaction takes place in the mixture in step (a) under 15 to 25° C., or optionally under 20 to 22° C.

237. The method of any one of embodiments 212 to 236, wherein a clicking chemistry reaction takes place in the mixture in step (a) for about 2 hours.

238. The method of any one of embodiments 212 to 237, wherein molar ratio of the cysteine added in step (b) for quenching the conjugation reaction to the phospholipid-PEG molecules comprising a bioconjugation linker is at least 3.

239. The method of embodiment 238, wherein the molar ratio is about 3.1 to about 4.1.

240. The method of any one of embodiments 212 to 239, wherein the quenching in step (b) is carried out for about 30 min.

241. The method of any one of embodiments 212 to 240, wherein the quenching in step (b) takes place under 15 to 25° C., or optionally under 20 to 22° C.

242. The method of any one of embodiments 212 to 241, wherein the method further comprises purifying the obtained composition comprising the phospholipid-PEG ISVD conjugate using ultrafiltration/diafiltration (UF/DF).

243. The method of embodiment 242, wherein the UF/DF has a molecular weight cut-off of about 10 kDa.

244. The method of any one of embodiments 212 to 243, wherein the composition comprising the phospholipid-PEG ISVD conjugate is formulated in buffer.

245. The method of embodiment 244, wherein the buffer comprises HEPES pH7.4, NaCl, and sucrose.

246. The method of embodiment 245, wherein the buffer comprises 1.5 mM HEPES pH7.4, 150 mM NaCl, and 10% sucrose buffer.

247. A phospholipid-PEG-ISVD conjugate produced by the method of any one of embodiments 212 to 246.

248. A composition comprising a phospholipid-PEG-ISVD conjugate produced by the method of any one of embodiments 212 to 246.

249. The composition of embodiment 248, wherein the composition comprises micelles comprising the phospholipid-PEG-ISVD conjugate.

250. The composition of embodiment 249, wherein the micelles comprise the phospholipid-PEG-ISVD conjugate, and a second phospholipid-PEG molecule that does not have a bioconjugation linker.

251. The composition of embodiment 250, wherein the micelles comprise DSPE-PEG 3.4K-anti-CD8α ISVD conjugate, and DSPE-PEG2.0k-OMeH.

252. The composition of embodiment 251, wherein the molar ratio among the CD8α ISVD, DSPE-PEG 3.4K, and DSPE-PEG2.0K is about 1:1:4 or 1:2:3.

253. The composition of anyone of embodiments 248 to 252, wherein the CD8α ISVD comprises or consists of SEQ ID NO: 44.

254. A method of producing a composition comprising lipid nanoparticles (LNPs), wherein the LNPs comprising:
(a) a lipid-immune cell targeting group conjugate comprising the compound of Formula (II): [Lipid]-[optional linker]-[antibody],
(b) an ionizable cationic lipid,
(c) a nucleic acid, wherein the nucleic acid is encapsulated in the LNP,
(d) a structural lipid (e.g., a sterol),
(e) a neutral phospholipid, and
(f) a free PEG-lipid,
wherein the method comprises:
(i) producing a first composition comprising the lipid-immune cell targeting group conjugate in (a);
(ii) producing a second composition comprising (b) to (f);
(iii) incubating the first composition obtained from step (i) and the second composition obtained from step (ii), to produce the final composition comprising the LNPs.

255. The method of embodiment 254, wherein the antibody in the lipid-immune cell targeting group conjugate comprises an ISVD.

256. The method of embodiment 254, wherein the lipid-immune cell targeting group conjugate is a phospholipid-PEG-ISVD.

257. The method of embodiment 256, wherein the phospholipid-PEG-ISVD is produced by the method of any one of embodiments 212 to 246.

258. The method of embodiment 257, wherein the phospholipid-PEG-ISVD is DSPE-PEG3.4K-ISVD.

259. The method of embodiment 258, wherein the ISVD is an anti-CD8 ISVD.

260. The method of embodiment 259, wherein the anti-CD8 ISVD comprises a sequence selected from the group consisting of SEQ ID NOs: 160 to 169, and SEQ ID NOs: 28-36 and 44, and SEQ ID NOs: 10 to 27.

261. The method of embodiment 260, wherein the anti-CD8 ISVD comprises SEQ ID NO: 44.

262. The method of embodiment 261, wherein the LNPs comprises Lipid 15, DSPC, Cholesterol, DPG-PEG, DSPE-PEG3.4K-A044300805_v8_GGC (SEQ ID NO: 44), and mRNA encoding a CD22 CAR.

263. The method of embodiment 262, wherein the mRNA comprises SEQ ID NO: 139 or SEQ ID NO: 147.

264. The method of embodiment 263, wherein the mRNA is produced through in vitro transcription.

265. The method of embodiment 264, wherein the mRNA comprises pseudouridine.

266. The method of embodiment 265, wherein the pseudouridine is N1-methyl-pseudouridine.

267. A composition produced by a method of any one of embodiments 254 to 266.

268. An isolated polynucleotide encoding any polypeptide of the present disclosure.

269. The isolated polynucleotide of embodiment 268, wherein the isolated polynucleotide is selected from the group consisting of:
(1) a polynucleotide encoding a polypeptide of the present disclosure, or a polypeptide having at least at least 70%, at least 75%, at least 80%, at least 85%, at least 90%, at least 91%, at least 91%, at least 91%, at least 91%, at least 91%, at least 91%, at least 91%, at least 91%, at least 91%, or more identity to a polypeptide sequence of the present disclosure;
(2) a polynucleotide having at least 90%, at least 91%, at least 91%, at least 91%, at least 91%, at least 91%, at least 91%, at least 91%, at least 91%, at least 91%, or more identity to a polynucleotide sequence of the present disclosure:
(3) a polynucleotide that hybridizes to a polynucleotide of the present disclosure under stringent conditions; and
(4) a polynucleotide that is a complement sequence of a polynucleotide of the present disclosure.

270. The isolated polynucleotide of embodiment 268 or 268, wherein the polynucleotide is selected from the group consisting of:
(1) a polynucleotide encoding a polypeptide having a sequence selected from SEQ ID Nos: 1 to 36, 44, 97-107, 116 to 123, 126-127, 160-179, 523-537, and 545-564, or a sequence having at least at least 70%, at least 75%, at least 80%, at least 85%, at least 90%, at least 91%, at least 91%, at least 91%, at least 91%, at least 91%, at least 91%, at least 91%, at least 91%, at least 91%, or more identity thereof.
(2) a polynucleotide of any one of SEQ ID Nos: 46-49, 50-53, 54-96, 108-115, 124-125, 128-148, and 159, or a sequence having at least 90%, at least 91%, at least 91%, at least 91%, at least 91%, at least 91%, at least 91%, at least 91%, at least 91%, at least 91%, or more identity to any of SEQ ID Nos: 46-49, 50-53, 54-96, 108-115, 124-125, 128-148, and 159;
(3) a polynucleotide that hybridizes to a polynucleotide of any of SEQ ID Nos: 46-49, 50-53, 54-96, 108-115, 124-125, 128-148, and 159 under stringent conditions; and
(4) a polynucleotide that is a complement sequence of a polynucleotide of any of SEQ ID Nos: 46-49, 50-53, 54-96, 108-115, 124-125, 128-148, and 159.

271. The isolated polynucleotide of any of embodiments 268 to 270, wherein the polynucleotide is a DNA molecule or an mRNA molecule.

272. The isolated polynucleotide of embodiment 271, wherein the polynucleotide is an mRNA molecule, and at least 5%, at least 10%, at least 20%, at least 30%, at least 40%, at least 50%, at least 60%, at least 70%, at least 80%, at least 90%, at least 95%, at least 99%, or more of the uridines are replaced with a pseudouridine.

273. The isolated polynucleotide of embodiment 272, wherein the pseudouridine is N1-methyl-pseudouridine.

274. An expression construct comprising a polynucleotide of any one of embodiments 268 to 273.

275. A vector, comprising the expression construction of embodiment 274.

276. A host cell comprising the expression construct of embodiment 275.

277. A composition comprising the isolated polynucleotide of embodiment 268 to 273, the expression construct of embodiment 274, the vector of embodiment 275, or the host cell of embodiment of 276.

1-61. (canceled)

62. A lipid nanoparticle (LNP) comprising: 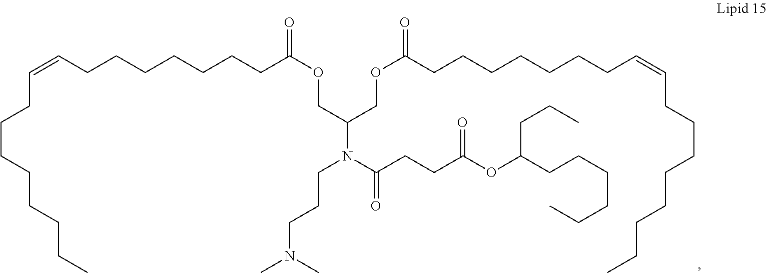 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]
(a) an ionizable cationic lipid: or a salt thereof;
(b) cholesterol;
(c) 1,2-distearoyl-sn-glycero-3-phosphocholine (DSPC);
(d) PEG 2000-dipalmitoyl-glycerol (DPG-PEG2K); and
(e) a lipid-immune cell targeting group conjugate comprising the compound of formula distearoyl-phosphatidylethanolamine (DSPE)-polyethylene glycol (PEG)-antibody conjugate, wherein the DSPE is covalently coupled to the antibody via a linker comprising PEG,
wherein the antibody is an immunoglobulin single variable domain (ISVD) that specifically binds to human CD8alpha, comprising complementarity-determining regions 1 (CDR1), 2 (CDR2), and 3 (CDR3) of an ISVD having the sequence of SEQ ID NO: 169,
wherein the LNP encapsulates an mRNA, and the mRNA encodes a polypeptide comprising a chimeric antigen receptor (CAR),
wherein the CAR comprises an antibody that specifically binds to human CD22,
wherein the antibody that specifically binds to human CD22 is an anti-CD22 ScFv, and
wherein the anti-CD22 ScFv comprises a CDR1, a CDR2, and a CDR3 of a heavy chain variable region (V_{H}) comprising the amino acid sequence of SEQ ID NO: 523, and a CDR1, CDR2, and a CDR3 of a light chain variable region (V_{L}) comprising the amino acid sequence of SEQ ID NO: 524.

63. The LNP of claim 62, wherein the PEG in the DSPE-PEG-antibody conjugate is PEG 3400 (PEG 3.4K).

64. The LNP of claim 63, wherein the ISVD that specifically binds to human CD8alpha comprises amino acid sequence of SEQ ID NO: 169 or SEQ ID NO: 44.

65. The LNP of claim 64, wherein the ISVD that specifically binds to human CD8alpha comprises amino acid sequence of SEQ ID NO: 44.

66. The LNP of claim 65, wherein the mRNA comprises the following elements, arranged from 5′ to 3′:
(1) optionally, a 5′ cap;
(2) optionally, a 5′ UTR;
(3) optionally, nucleotides encoding a Lead peptide sequence;
(4) nucleotides encoding the VH of the anti-CD22 ScFv;
(5) optionally, nucleotides encoding a Linker A;
(6) nucleotides encoding the VL of the anti-CD22 ScFv;
(7) nucleotides encoding a Linker B;
(8) nucleotides encoding a Hinge domain;
(9) nucleotides encoding a Transmembrane domain;
(10) nucleotides encoding a Co-stimulatory domain;
(11) nucleotides encoding a Signaling domain;
(12) optionally, a 3′ UTR; and
(13) optionally, a polyA tail.

67. The LNP of claim 66, wherein the mRNA comprises the following elements, arranged from 5′ to 3′:
(1) a 5′ cap;
(2) a 5′ UTR;
(3) nucleotides encoding a Lead peptide sequence;
(4) nucleotides encoding the VH of the anti-CD22 ScFv;
(5) nucleotides encoding a Linker A;
(6) nucleotides encoding the VL of the anti-CD22 ScFv;
(7) nucleotides encoding a Linker B;
(8) nucleotides encoding a Hinge domain;
(9) nucleotides encoding a Transmembrane domain;
(10) nucleotides encoding a Co-stimulatory domain;
(11) nucleotides encoding a Signaling domain;
(12) a 3′ UTR; and
(13) a polyA tail.

68. The LNP of claim 67, wherein the anti-CD22 ScFv comprises a VH comprising the amino acid sequence of SEQ ID NO: 523 and a VL comprising the amino acid sequence of SEQ ID NO: 524.

69. The LNP of claim 68, wherein the Lead peptide comprises SEQ ID NO: 515.

70. The LNP of claim 68, wherein the Linker A is (GGGGS) 4 (SEQ ID NO: 344).

71. The LNP of claim 68, wherein the Linker B is AAA.

72. The LNP of claim 68, wherein the Hinge domain and the Transmembrane domain are derived from CD28 hinge and transmembrane domains.

73. The LNP of claim 72, wherein the Hinge domain comprises or consists of SEQ ID NO: 541.

74. The LNP of claim 72, wherein the Transmembrane domain comprises or consists of SEQ ID NO: 542.

75. The LNP of claim 68, wherein the Co-stimulatory domain is a CD28 Co-stimulatory domain.

76. The LNP of claim 75, wherein the Co-stimulatory domain comprises or consists of SEQ ID NO: 543.

77. The LNP of claim 68, wherein the Signaling domain is derived from a CD3z signaling domain.

78. The LNP of claim 77, wherein the Signaling domain comprises or consists of SEQ ID NO: 544.

79. The LNP of claim 68, wherein the mRNA encodes the polypeptide of SEQ ID NO: 126.

80. The LNP of claim 79, wherein the mRNA comprises the sequence of SEQ ID NO: 108.

81. The LNP of claim 80, wherein the 5′ cap of the mRNA is Cap 1, the 5′ UTR comprises or consists of SEQ ID NO: 518, and the 3′ UTR comprises or consists of SEQ ID NO: 519.

82. The LNP of claim 81, wherein the mRNA comprises pseudouridine.

83. The LNP of claim 82, wherein the pseudouridine is N1-methyl-pseudouridine.

84. The LNP of claim 83, wherein 100% of uridines in the mRNA are replaced with N1-methyl-pseudouridine.

85. The LNP of claim 84, wherein the ionizable cationic lipid in the LNP has a concentration of between about 45 mol % and 55 mol % of the LNP.

86. The LNP of claim 84, wherein the cholesterol has a concentration of between about 25 mol % and about 45 mol % of the LNP.

87. The LNP of claim 84, wherein the DSPC has a concentration of between about 5 mol % and about 25% mol % of the LNP.

88. The LNP of claim 84, wherein the DPG-PEG2K has a concentration of between about 0.5 mol % and about 2.5 mol % of the LNP.

89. The LNP of claim 84, wherein
(i) the cationic lipid has a concentration of about 49.2 mol % of the LNP;
(ii) the cholesterol has a concentration of about 29.3 mol % of the LNP;
(iii) the DSPC has a concentration of about 20.0 mol % of the LNP; and
(iv) the DPG-PEG2K has a concentration of about 1.5 mol % of the LNP.

90. The LNP of claim 84, wherein
(i) the cationic lipid has a concentration of about 14.2 g/g mRNA in the LNP;
(ii) the cholesterol has a concentration of about 3.45 g/g mRNA in the LNP;
(iii) the DSPC has a concentration of about 4.84 g/g mRNA in the LNP;
(iv) the DPG-PEG2K has a concentration of about 1.15 g/g mRNA in the LNP; and
(v) the DSPE-PEG3.4K-anti-CD8 antibody conjugate has a concentration of about 0.084 g/g to 0.15 g/g mRNA in the LNP.

91. The LNP of claim 84, wherein the LNP comprises one or more additional components that are included in the LNP due to a manufacturing process that is used to produce the LNP, and at least one of the one or more additional components is a phospholipid-PEG that does not have a bioconjugation linker, and wherein the phospholipid-PEG that does not have a bioconjugation linker is DSPE-PEG 3.4K or DSPE-PEG 2K.
